(19) United States
(12) Patent Application Publication
(10) Pub. No.: US 2020/0274070 A1
(43) Pub. Date: Aug. 27, 2020

(54) COMPOUND AND ORGANIC ELECTRONIC DEVICE USING THE SAME

(71) Applicant: SHANGHAI NICHEM FINE CHEMICAL CO., LTD., SHANGHAI (CN)

(72) Inventors: Chi-Chung CHEN, Hsinchu County (TW); Shwu-Ju SHIEH, Hsinchu County (TW); Cheng-Pin CHEN, Hsinchu County (TW); Ming-Zer LEE, Hsinchu County (TW)

(21) Appl. No.: 16/796,161

(22) Filed: Feb. 20, 2020

Related U.S. Application Data
(60) Provisional application No. 62/811,241, filed on Feb. 27, 2019.

(51) Int. Cl. H01L 51/00; C07D 405/14; C07D 409/14

(52) U.S. Cl. CPC H01L 51/0067; C07D 405/14; H01L 51/5016; C07D 409/14; H01L 51/0074; H01L 51/0073

(57) ABSTRACT

Provided are a novel compound and an organic electronic device using the same. The novel compound is represented by the following Formula (I):

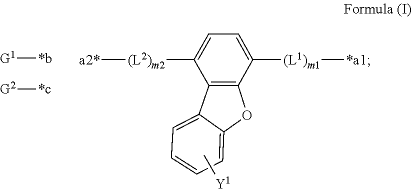

wherein *b is bonded to one of *a1 and a2*, and *c is bonded to the other of *a1 and a2*; L¹ to L² are each independently an arylene group having 6 to 60 ring carbon atoms; Y¹ is selected from the group consisting of: a hydrogen atom, a deuterium atom, an alkyl group having 1 to 12 carbon atoms, and an aryl group having 6 to 30 ring carbon atoms; and m1 to m2 are each independently an integer 0 or 1.

### CROSS-REFERENCE TO RELATED APPLICATION

Pursuant to 35 U.S.C. § 119(e), this application claims the benefits of the priority to U.S. Provisional Patent Application No. 62/811,241, filed Feb. 27, 2019. The contents of the prior application are incorporated herein by its entirety.

### BACKGROUND OF THE INVENTION

### 1. Field of the Invention

The present invention relates to a novel compound and an organic electronic device using the same, more particularly to a novel compound as an electron transport material or a hole blocking material and an organic electronic device using the same.

### 2. Description of the Prior Arts

With the advance of technology, various organic electronic devices that make use of organic materials have been energetically developed. Examples of the organic electronic devices include organic light emitting devices (OLEDs), organic phototransistors, organic photovoltaic cells, and organic photodetectors.

OLED was initially invented and proposed by Eastman Kodak Company through a vacuum evaporation method. Dr. Ching W. Tang and Steven VanSlyke of Kodak Company deposited an electron transport material such as tris(8-hydroxyquinoline)aluminum(III) (abbreviated as Alq₃) on a transparent indium tin oxide glass (abbreviated as ITO glass) formed with a hole transport layer of organic aromatic diamine thereon, and subsequently deposited a metal electrode onto an electron transport layer to complete the fabrication of the OLED. OLEDs have attracted lots of attention due to their numerous advantages, such as fast response speed, light weight, compactness, wide viewing angle, high brightness, higher contrast ratio, no need of backlight, and low power consumption. However, the OLEDs still have the problems such as short lifetime.

To overcome the problem of short lifetime, one of the approaches is to interpose some interlayers between the cathode and the anode. With reference to FIG. 1, a modified OLED 1 may have a structure of a substrate 11, an anode 12, a hole injection layer 13 (abbreviated as HIL), a hole transport layer 14 (abbreviated as HTL), an emission layer 15 (abbreviated as EL), an electron transport layer 16 (abbreviated as ETL), an electron injection layer 17 (abbreviated as EIL), and a cathode 18 stacked in sequence. When a voltage is applied between the anode 12 and the cathode 18, the holes injected from the anode 12 move to the EL via HIL and HTL and the electrons injected from the cathode 18 move to the EL via EIL and ETL. Recombination of the electrons and the holes occurs in the EL to generate excitons, thereby emitting a light when the excitons decay from excited state to ground state.

Another approach is to modify the materials of ETL for OLEDs to render the electron transport materials to exhibit hole-blocking ability. Examples of conventional electron transport materials include 2,9-dimethyl-4,7-diphenyl-1,10-phenanthroline (BCP), 3,3′-[5′-[3-(3-pyridinyl)phenyl][1,1′:3′,1″-terphenyl]-3,3″-diyl]bispyridine (TmPyPb), 1,3,5-tris(1-phenyl-1H-benzimidazol-2-yl)benzene (TPBi), tris(2,4,6-trimethyl-3-(pyridin-3-yl)phenyl)borane (3TPYMB), 1,3-bis(3,5-dipyrid-3-yl-phenyl)benzene (BmPyPb), and 9,10-bis(3-(pyridin-3-yl)phenyl)anthracene (DPyPA).

However, even using the foresaid electron transport materials, the lifespan of OLEDs still needs to be improved. Therefore, the present invention provides a novel compound to mitigate or obviate the problems in the prior art.

### SUMMARY OF THE INVENTION

An objective of the present invention is to provide a novel compound useful for an organic electronic device.

Another objective of the present invention is to provide an organic electronic device using the novel compound, so as to prolong the lifespan of the organic electronic device.

To achieve the foresaid objectives, the present invention provides a novel compound represented by the following Formula (I):

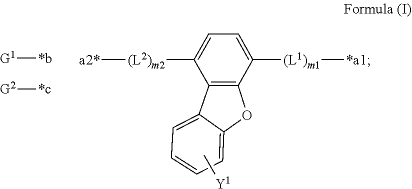

In Formula (I), *a1, a2*, *b, and *c represent bonding sites, *b is bonded to one of *a1 and a2*, and *c is bonded to the other of *a1 and a2*.

In Formula (I), G¹-*b is represented by

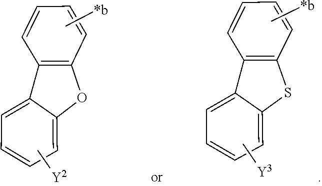

In Formula (I), G² is selected from the group consisting of:

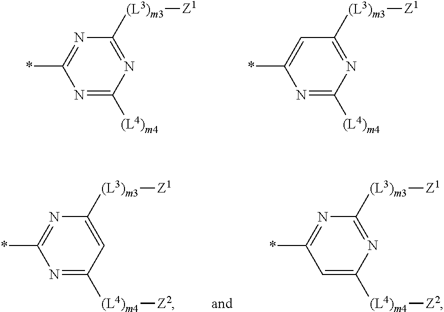

wherein Z¹ and Z² are each independently selected from the group consisting of: a substituted aryl group having 6 to 60 ring carbon atoms, an unsubstituted aryl group having 6 to 60 ring carbon atoms, a substituted heteroaryl group having 3 to 60 ring carbon atoms, and an unsubstituted heteroaryl group having 3 to 60 ring carbon atoms.

m1 to m4 are each independently an integer 0 or 1, and m1 to m4 are the same or different.

L¹ to L⁴ are each independently an arylene group having 6 to 60 ring carbon atoms, and L¹ to L⁴ are the same or different.

Y¹ to Y³ are each independently selected from the group consisting of: a hydrogen atom, a deuterium atom, an alkyl group having 1 to 12 carbon atoms, and an aryl group having 6 to 30 ring carbon atoms, and Y¹ to Y³ are the same or different.

Preferably, the compound may be represented by any one of the following Formulae (I-I) to (I-XVI):

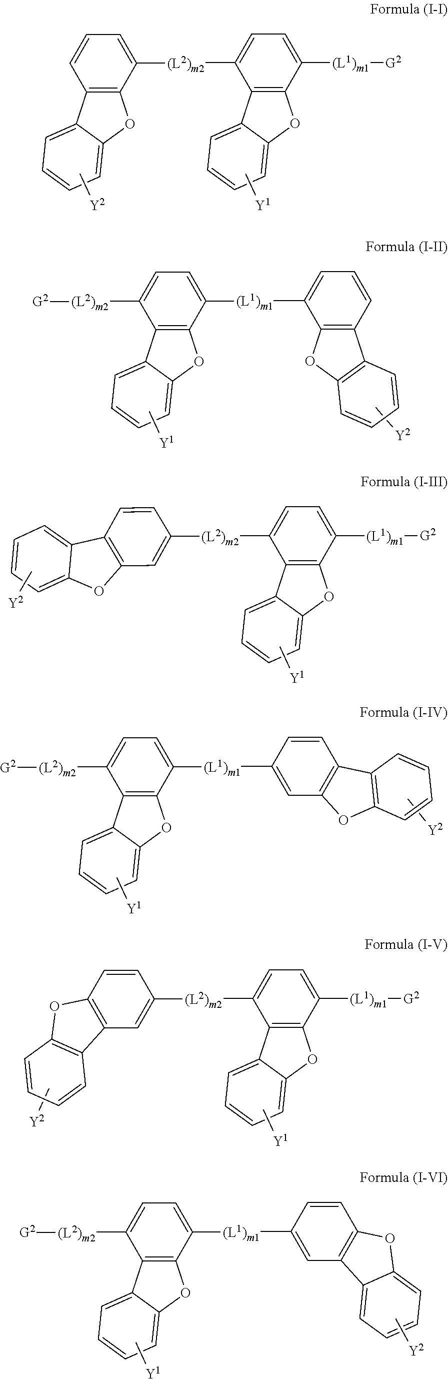

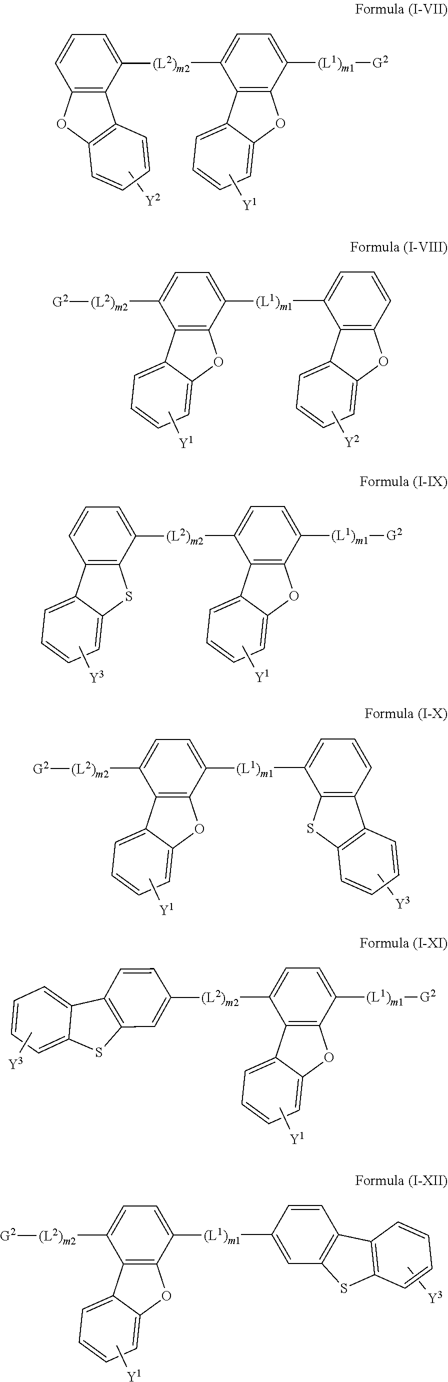

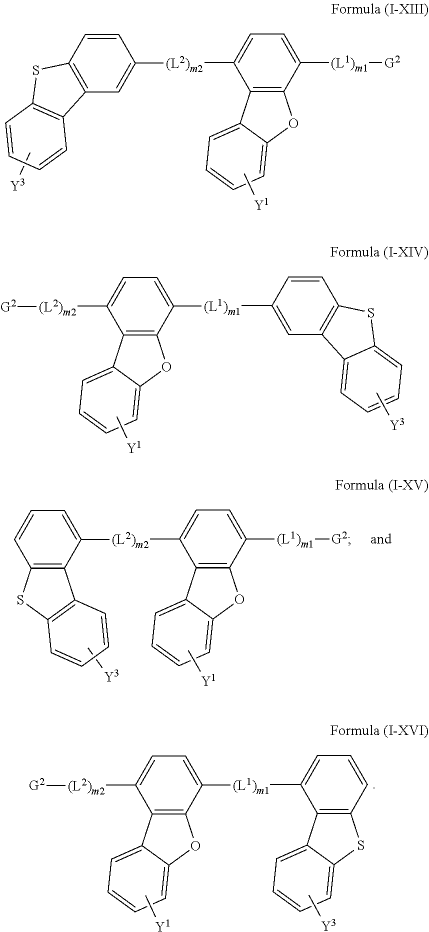

Preferably, said Z¹ and Z² are each independently selected from the group consisting of:

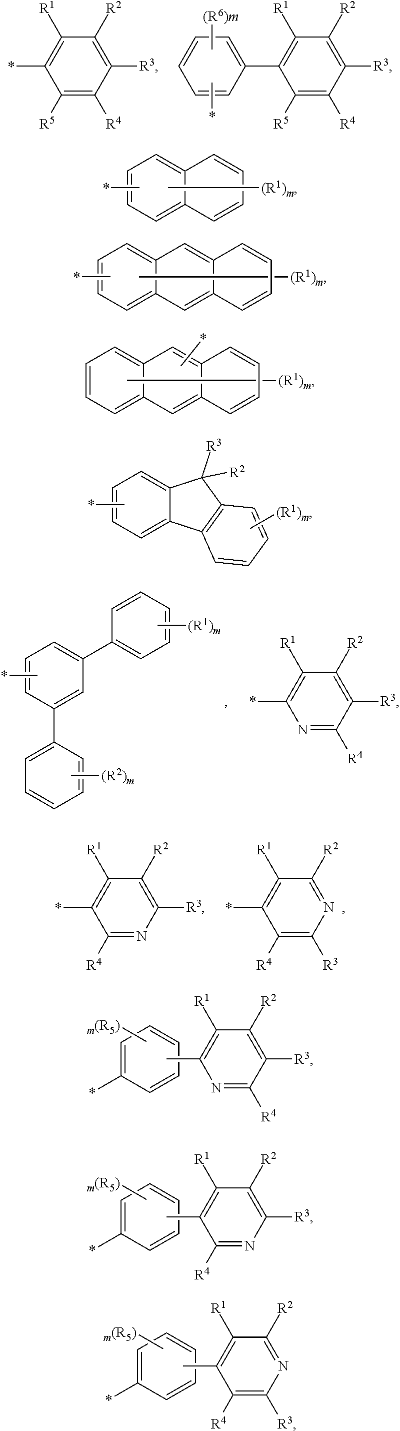

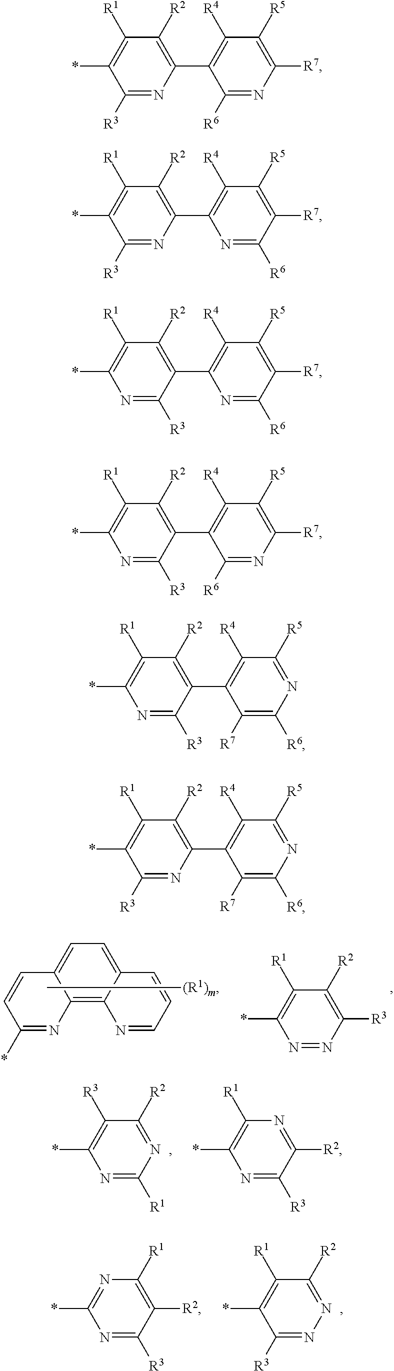

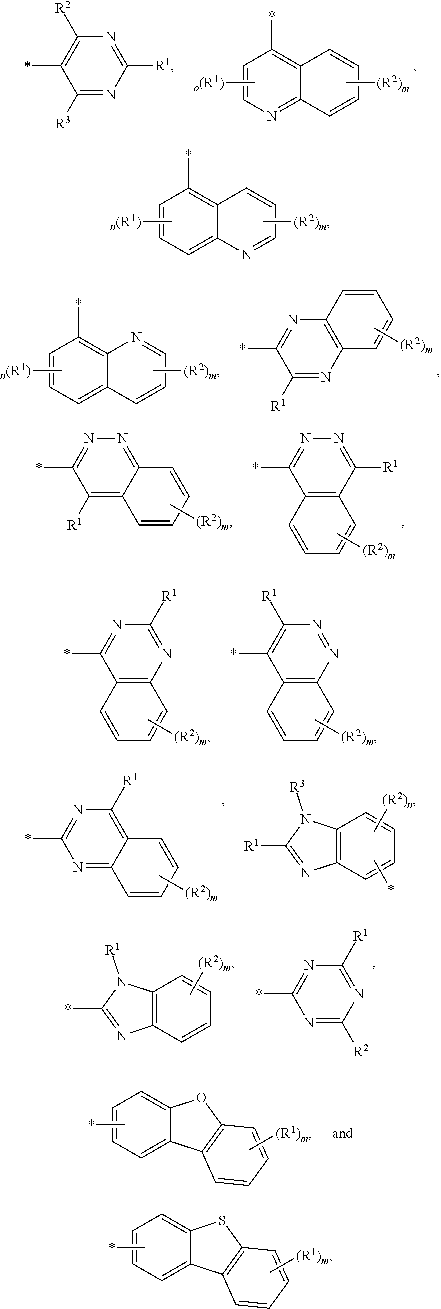

wherein R¹ to R⁷ are each independently selected from the group consisting of: a hydrogen atom, a deuterium atom, a halogen group, a cyano group, a nitro group, a trifluoromethyl group, an unsubstituted alkyl group having 1 to 12 carbon atoms, an alkyl group having 1 to 12 carbon atoms substituted with a substituent, an unsubstituted alkenyl group having 2 to 12 carbon atoms, an alkenyl group having 2 to 12 carbon atoms substituted with a substituent, an unsubstituted alkynyl group having 2 to 12 carbon atoms, an alkynyl group having 2 to 12 carbon atoms substituted with a substituent, an unsubstituted aryl group having 6 to 30 ring carbon atoms, an aryl group having 6 to 30 ring carbon atoms substituted with a substituent, an unsubstituted heteroaryl group having 3 to 30 ring carbon atoms, and a heteroaryl group having 3 to 30 ring carbon atoms substituted with a substituent, wherein the substituent is selected from the group consisting of: a deuterium atom, a halogen group, a cyano group, a nitro group, and a trifluoromethyl group.

m is an integer from 1 to 4, n is an integer from 1 to 3, and o is an integer 1 or 2.

Preferably, said Z¹ is selected from the group consisting of:

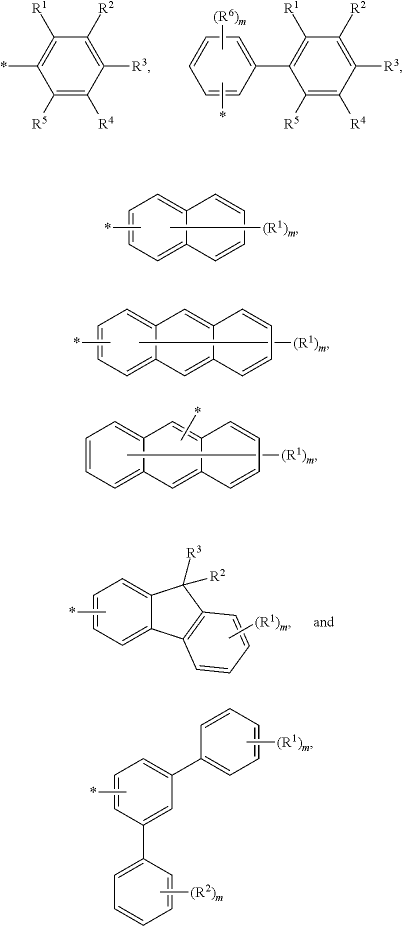

said Z² is selected from the group consisting of:

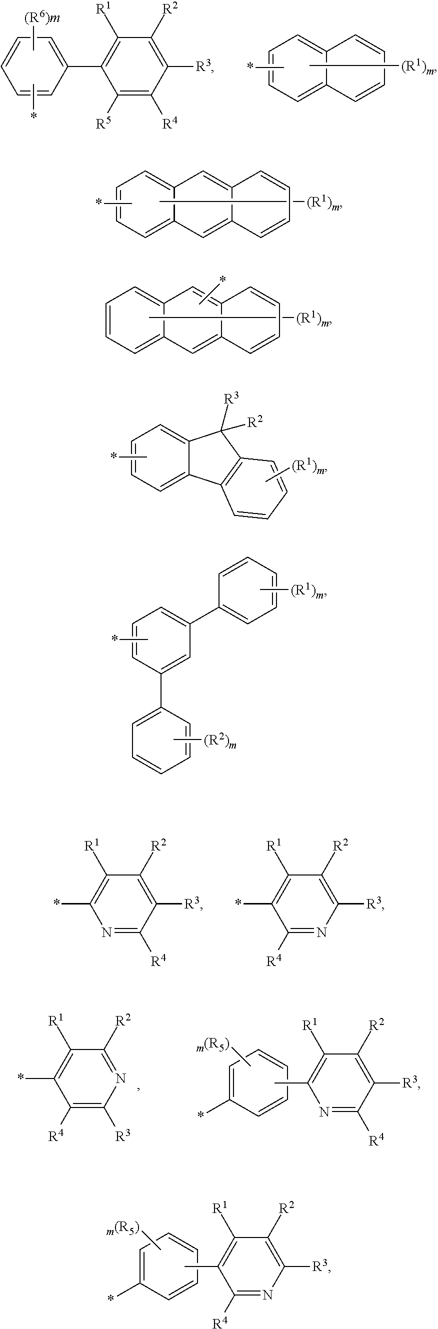

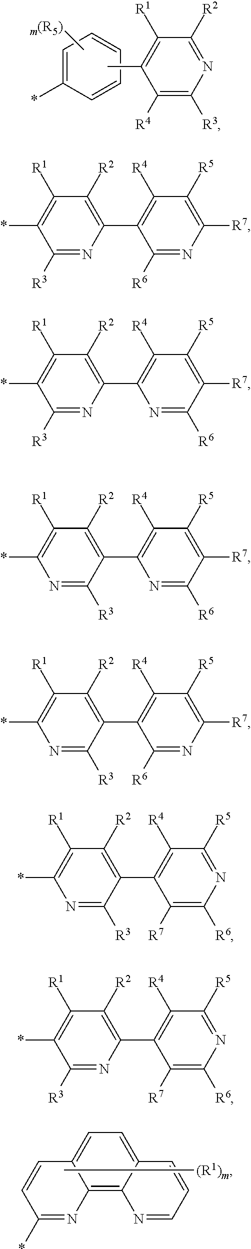

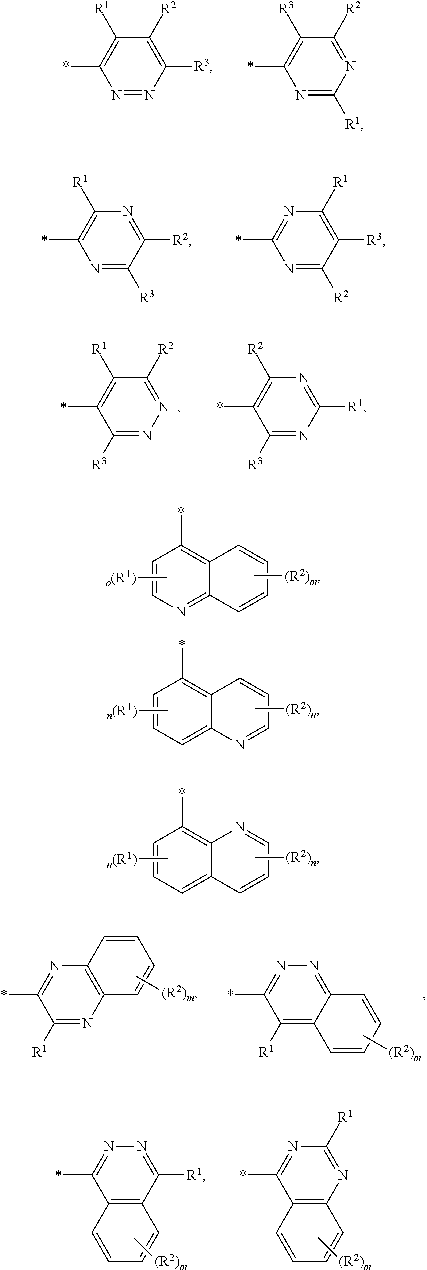

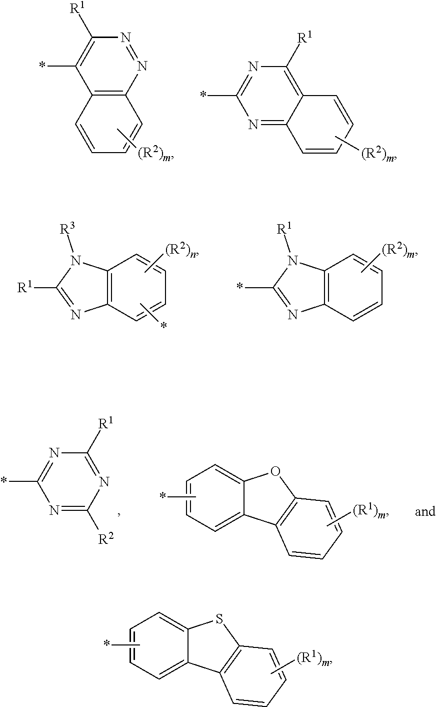

wherein R¹ to R⁷ are each independently selected from the group consisting of: a hydrogen atom, a deuterium atom, a halogen group, a cyano group, a nitro group, a trifluoromethyl group, an unsubstituted alkyl group having 1 to 12 carbon atoms, an alkyl group having 1 to 12 carbon atoms substituted with a substituent, an unsubstituted alkenyl group having 2 to 12 carbon atoms, an alkenyl group having 2 to 12 carbon atoms substituted with a substituent, an unsubstituted alkynyl group having 2 to 12 carbon atoms, an alkynyl group having 2 to 12 carbon atoms substituted with a substituent, an unsubstituted aryl group having 6 to 30 ring carbon atoms, an aryl group having 6 to 30 ring carbon atoms substituted with a substituent, an unsubstituted heteroaryl group having 3 to 30 ring carbon atoms, and a heteroaryl group having 3 to 30 ring carbon atoms substituted with a substituent, wherein the substituent is selected from the group consisting of: a deuterium atom, a halogen group, a cyano group, a nitro group, and a trifluoromethyl group.

m is an integer from 1 to 4, n is an integer from 1 to 3, and o is an integer 1 or 2.

More preferably, said R¹ to R⁷ of said Z¹ and Z² are each independently selected from the group consisting of: a hydrogen atom, a deuterium atom, a halogen group, a cyano group, a nitro group, a trifluoromethyl group, a methyl group, an ethyl group, a propyl group, a butyl group, a pentyl group, a hexyl group, a phenyl group, a napthyl group, a biphenyl group, a triphenyl group, and a trifluoromethylphenyl group.

More specifically, said Z¹ and Z² are each independently selected from the group consisting of:

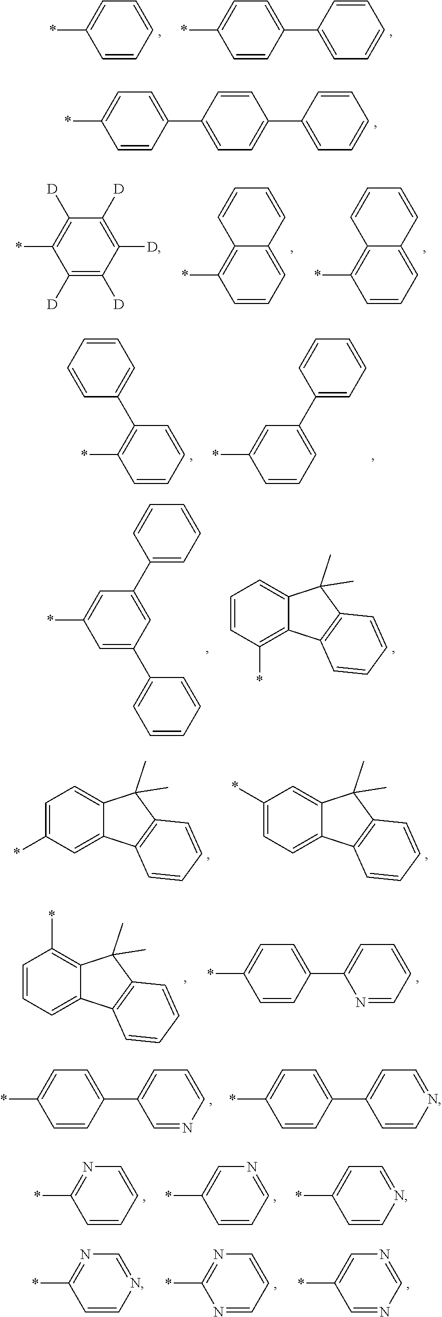

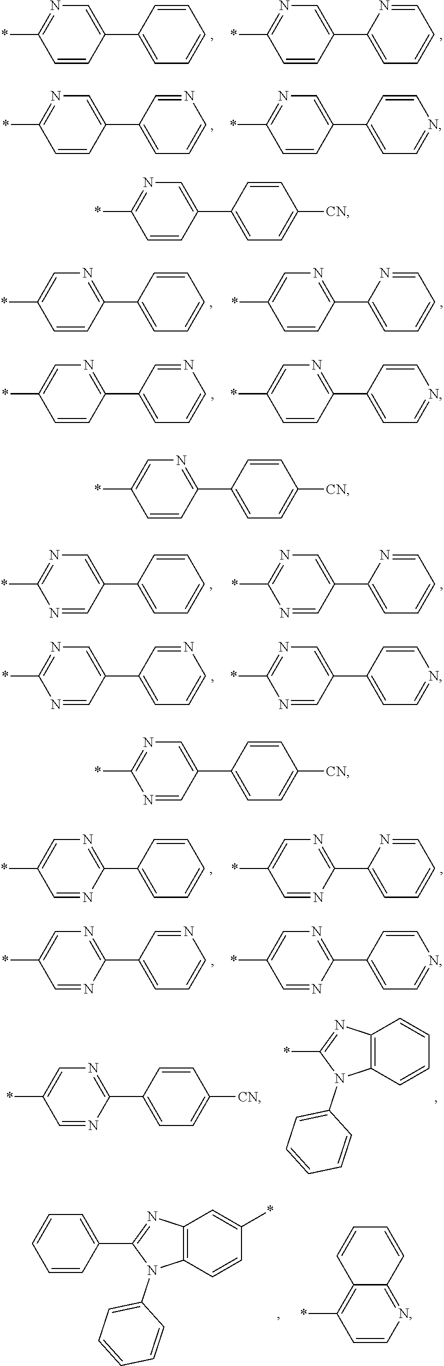

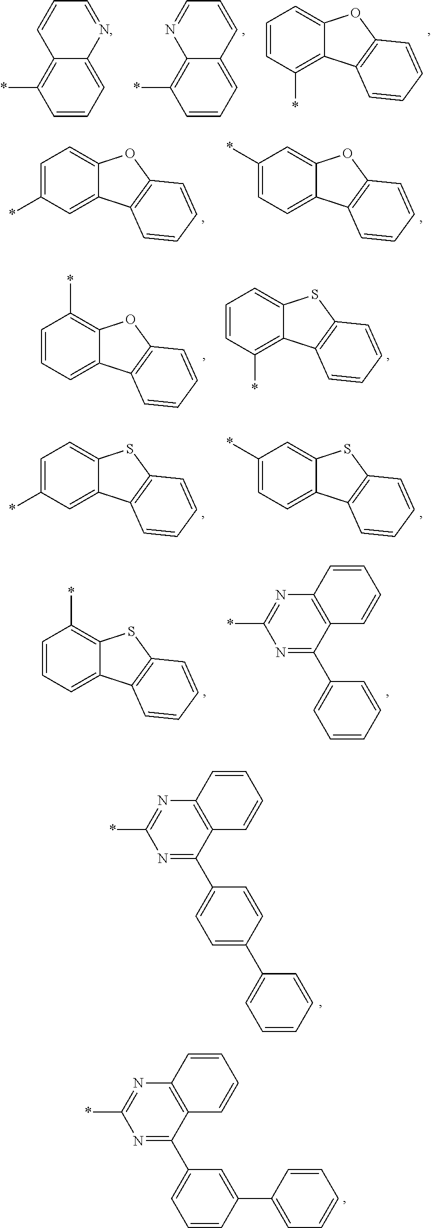

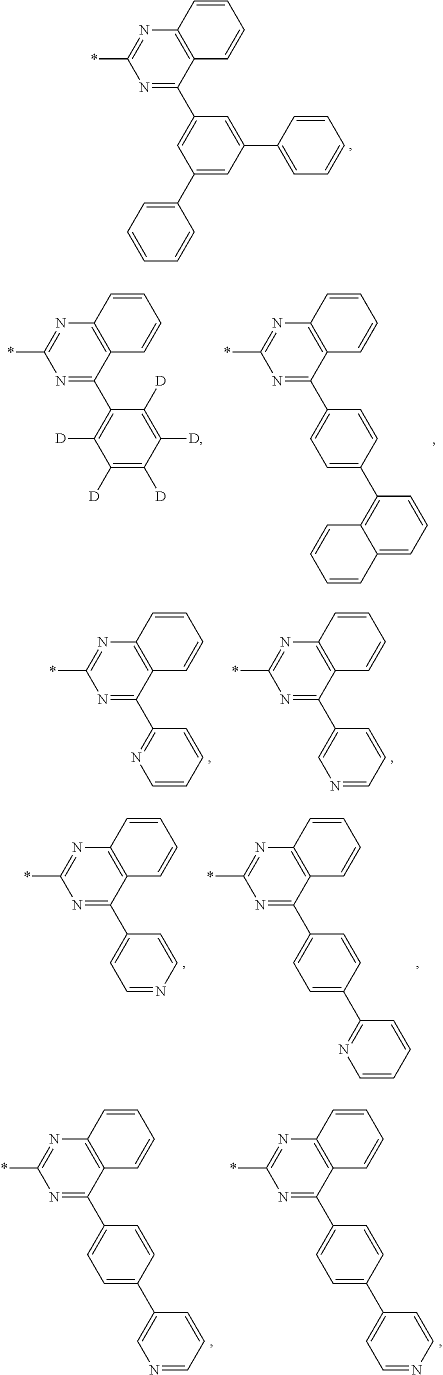

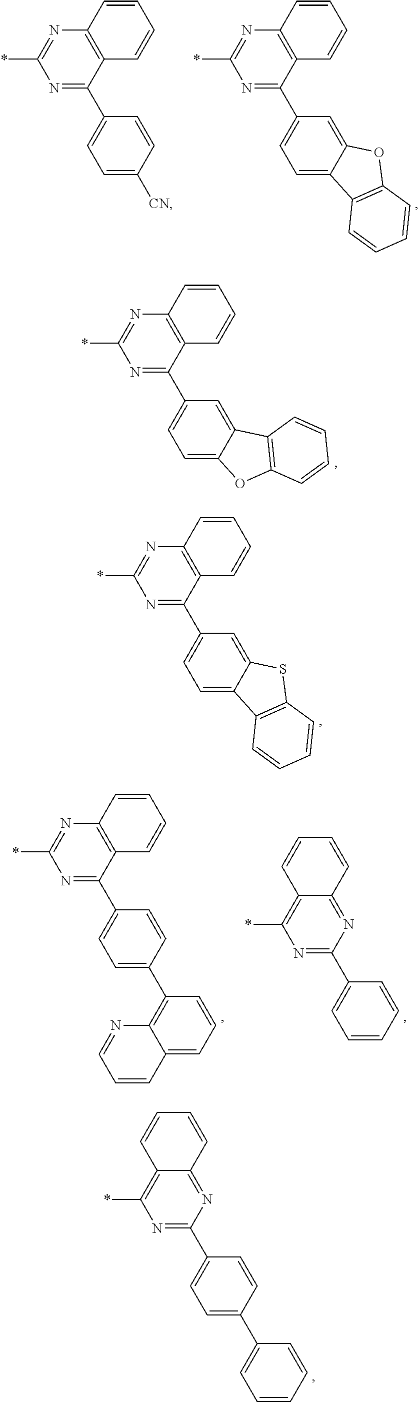

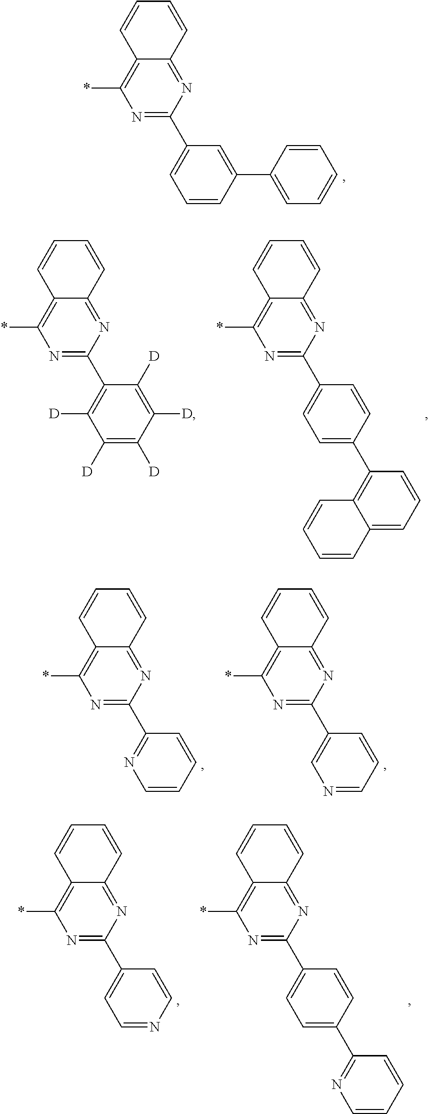

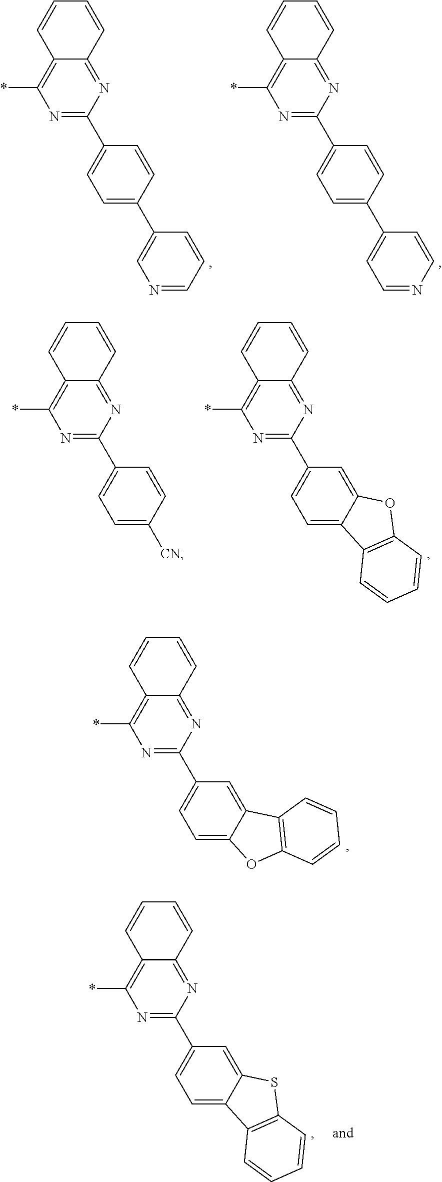 [SMILES: *c1nc(-c2ccc(-c3cccnc3)cc2)nc2ccccc12 ; *c1nc(-c2ccc(-c3ccncc3)cc2)nc2ccccc12 ; *c1nc(-c2ccc(C#N)cc2)nc2ccccc12 ; *c1nc(-c2ccc3c(c2)oc2ccccc23)nc2ccccc12 ; *c1nc(-c2ccc3oc4ccccc4c3c2)nc2ccccc12 ; *c1nc(-c2ccc3c(c2)sc2ccccc23)nc2ccccc12]

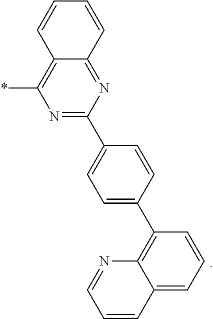 [SMILES: *c1nc(-c2ccc(-c3cccc4cccnc34)cc2)nc2ccccc12]

More specifically, said G² of Formula (I) is selected from the group consisting of:

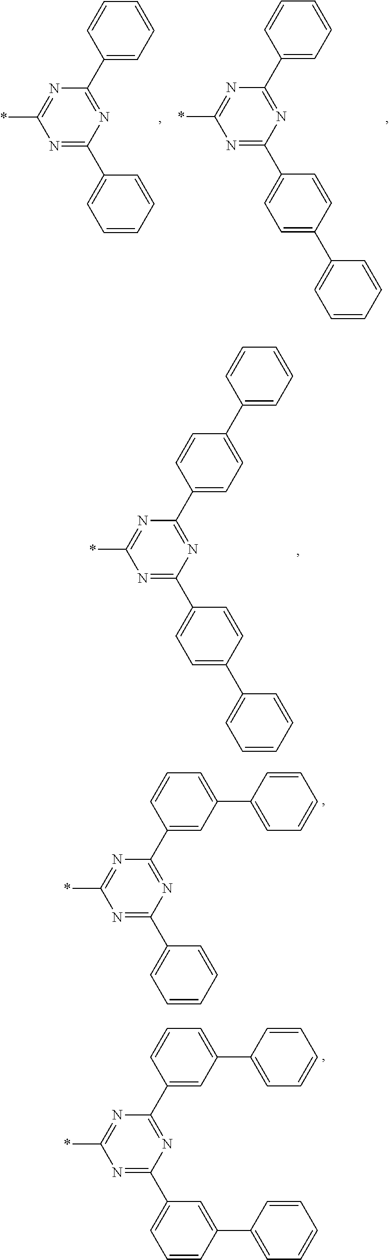

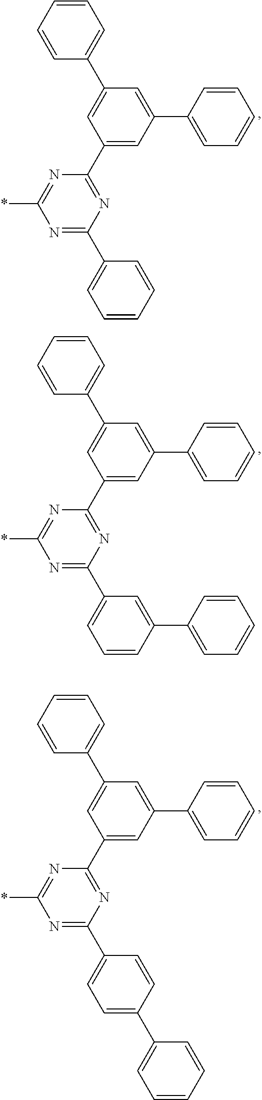

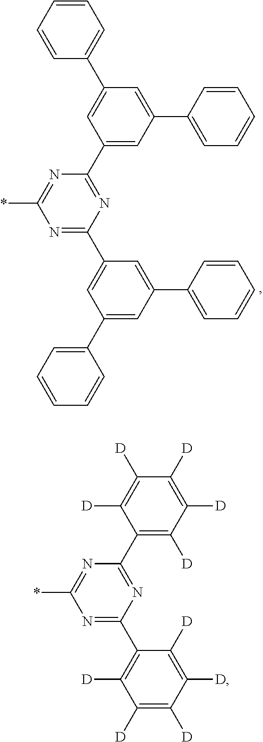

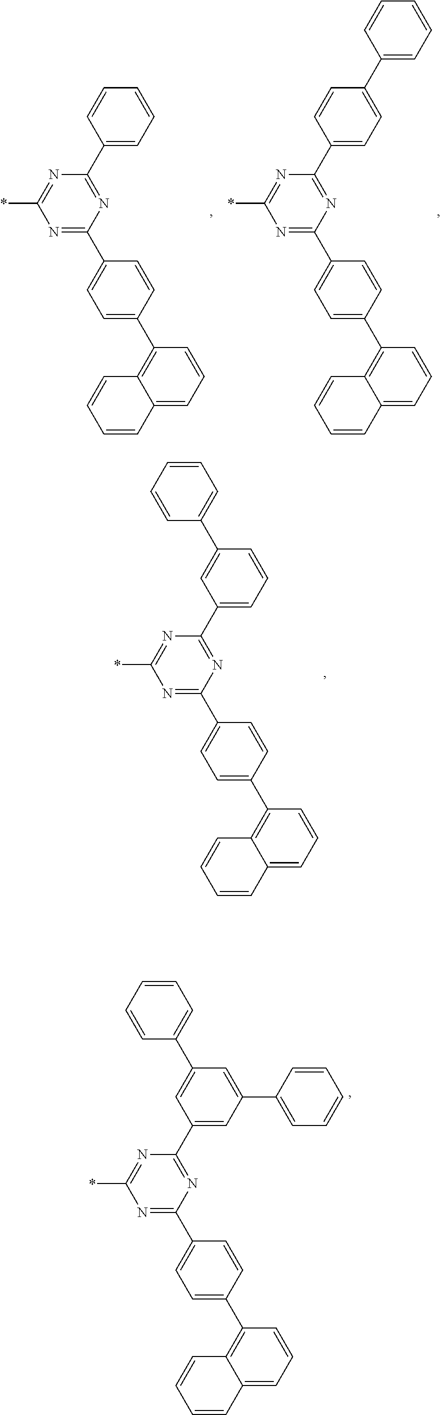

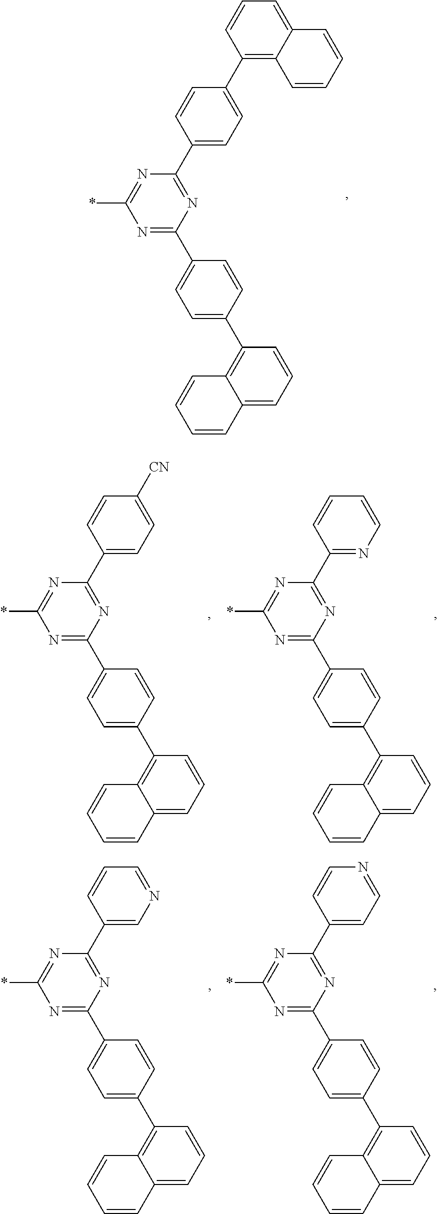

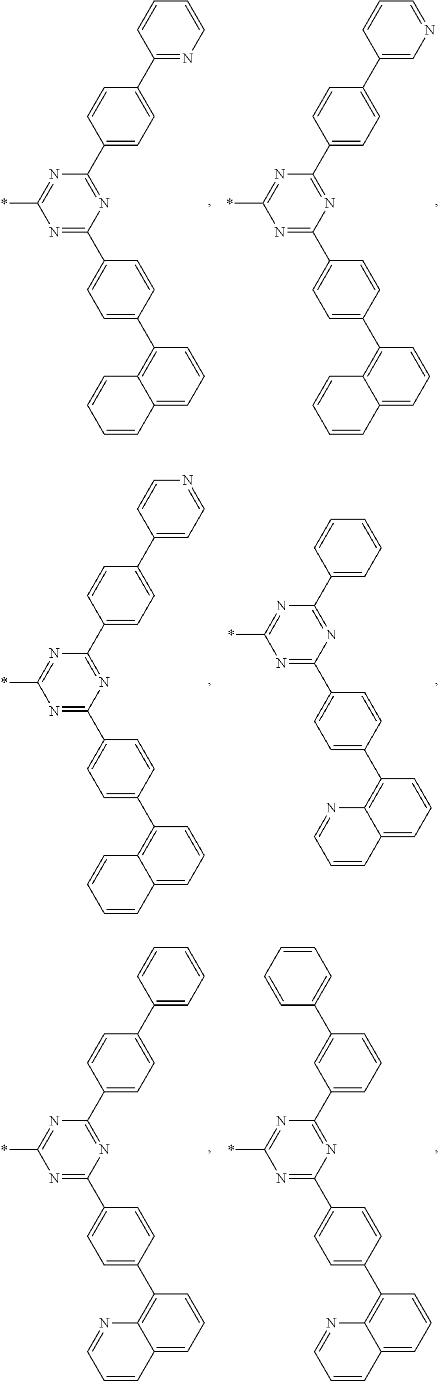

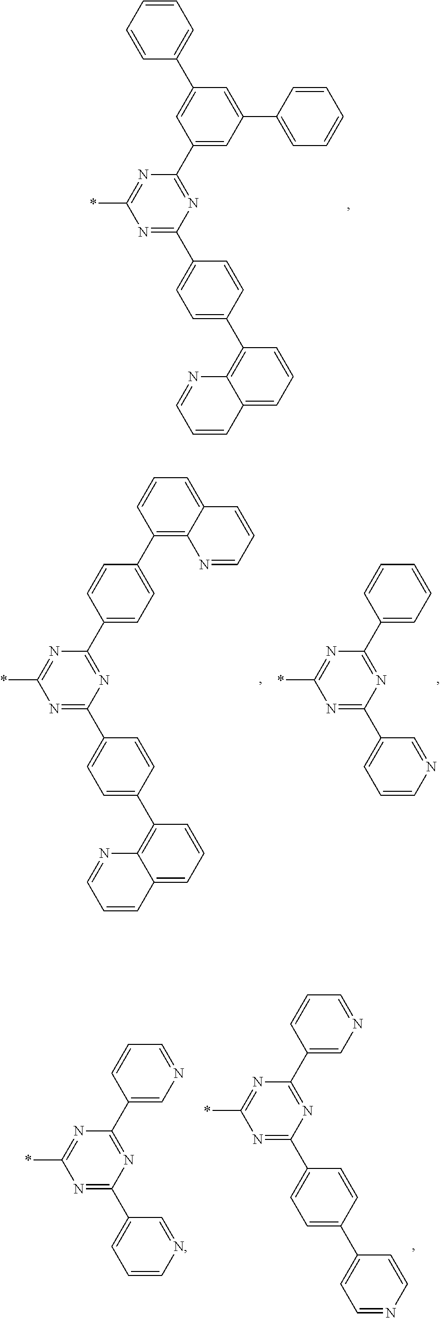

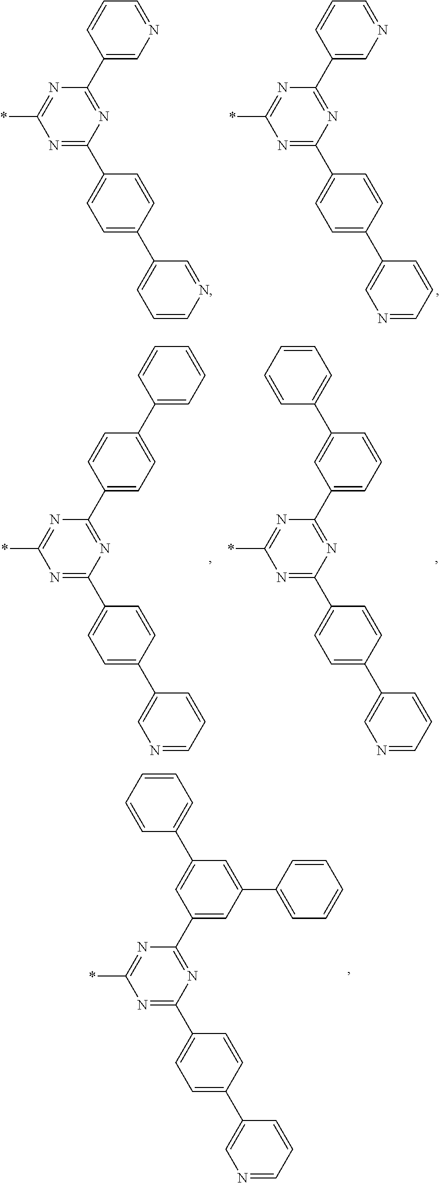

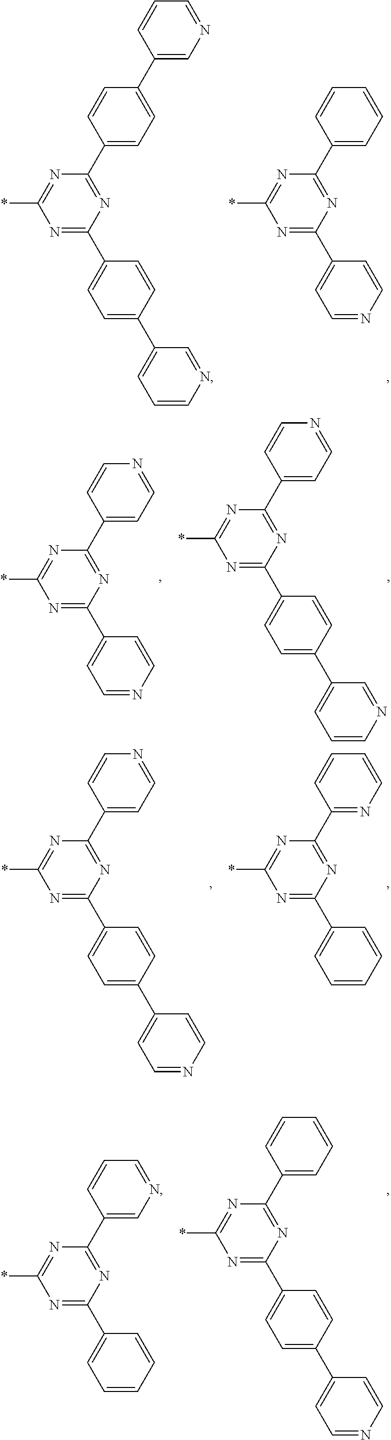

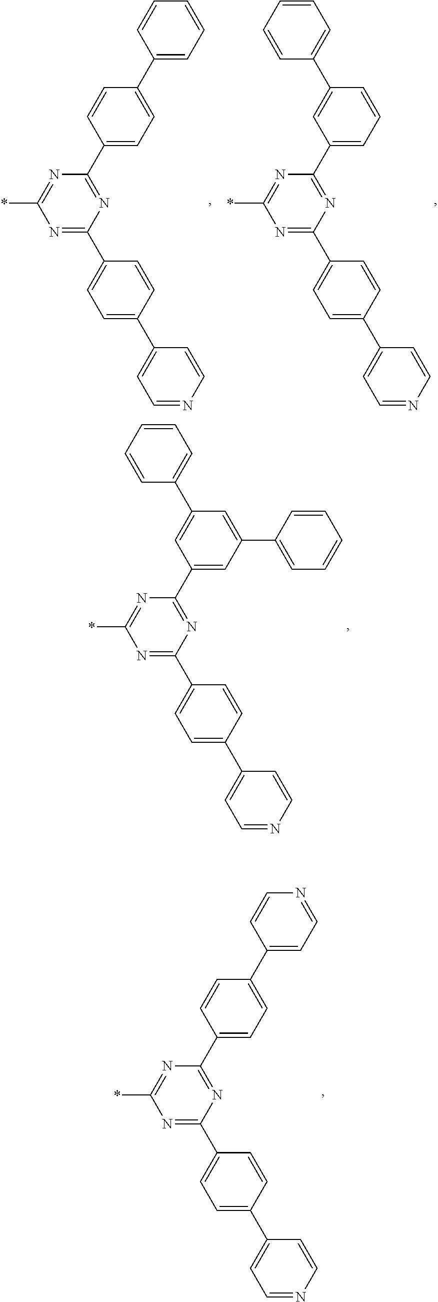

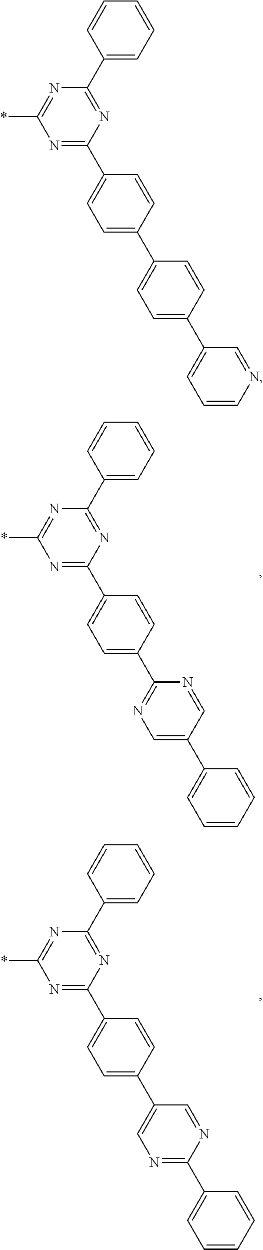 [SMILES: *c1nc(-c2ccccc2)nc(-c2ccc(-c3ccc(-c4cccnc4)cc3)cc2)n1 ; *c1nc(-c2ccccc2)nc(-c2ccc(-c3ncc(-c4ccccc4)cn3)cc2)n1 ; *c1nc(-c2ccccc2)nc(-c2ccc(-c3cnc(-c4ccccc4)nc3)cc2)n1]

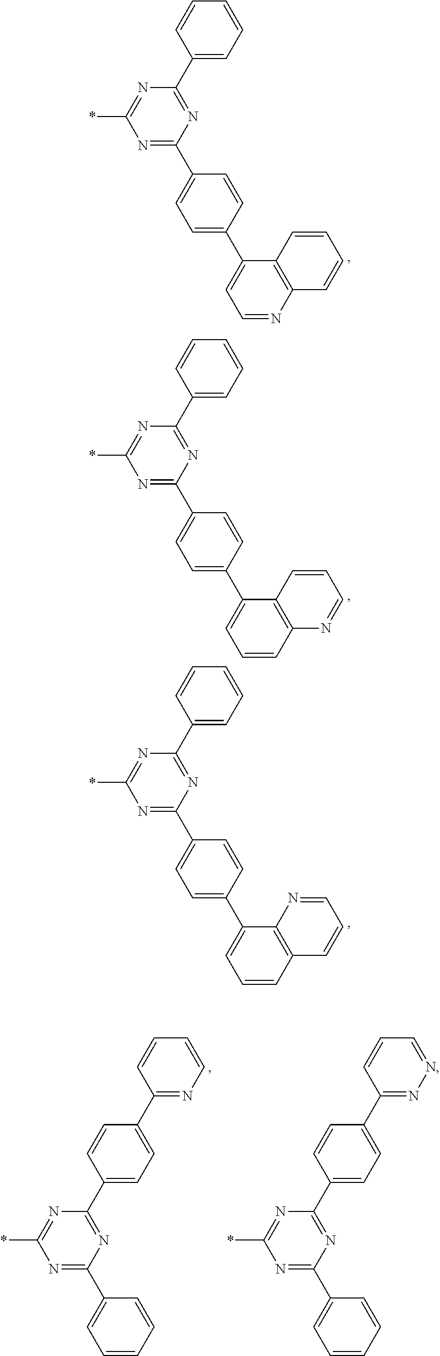

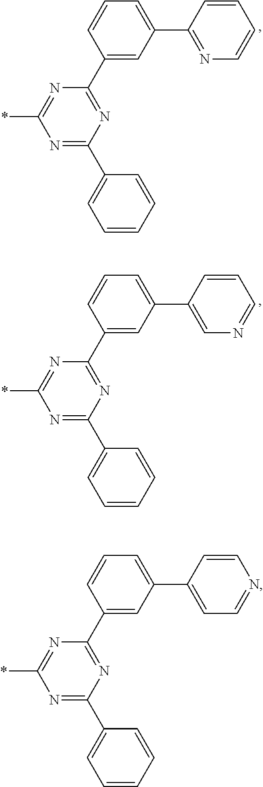

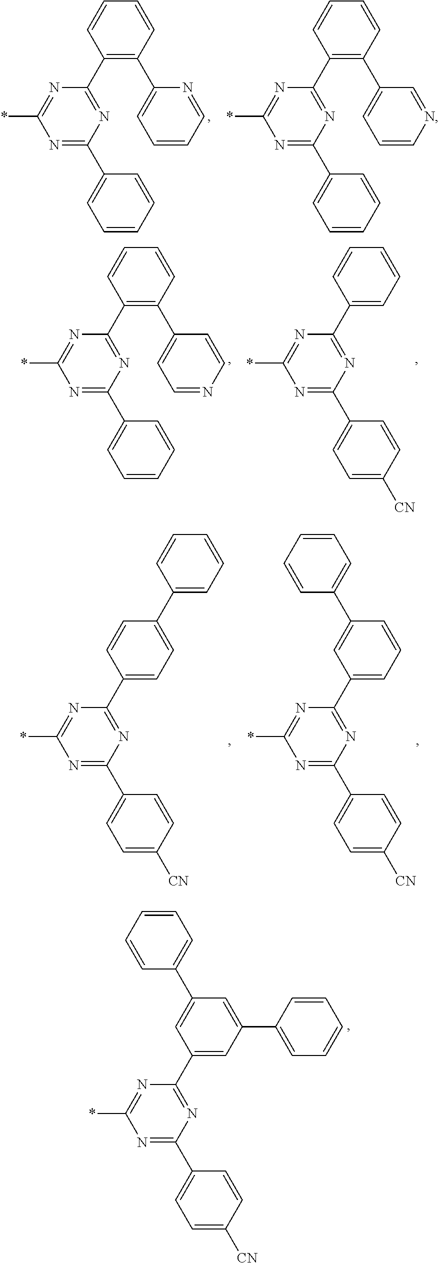

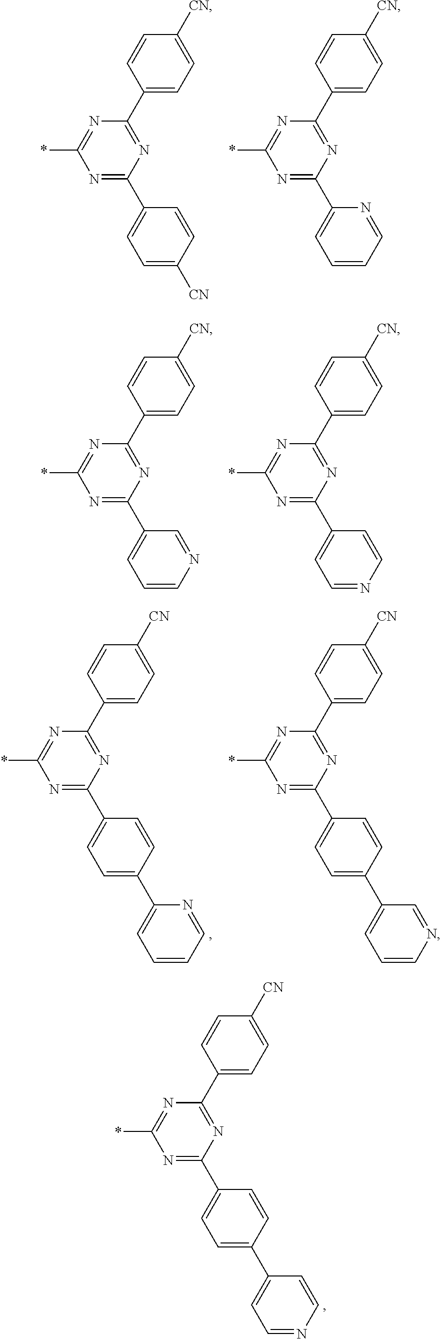

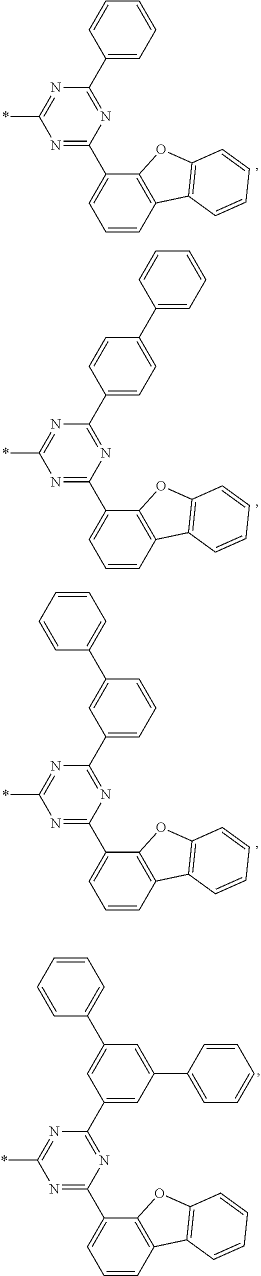

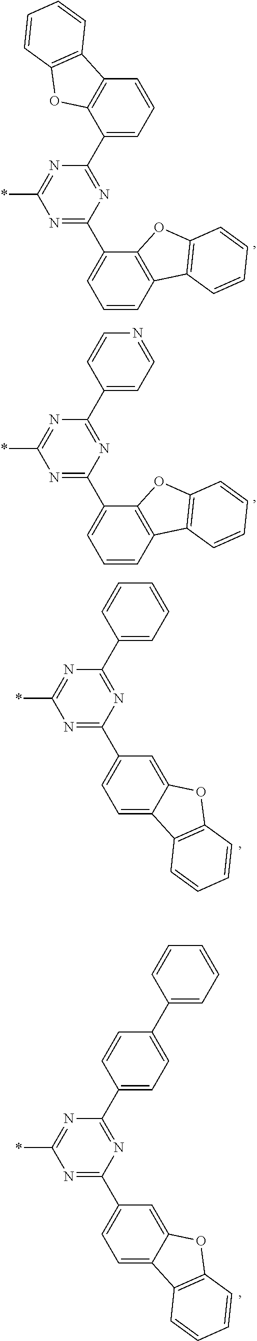

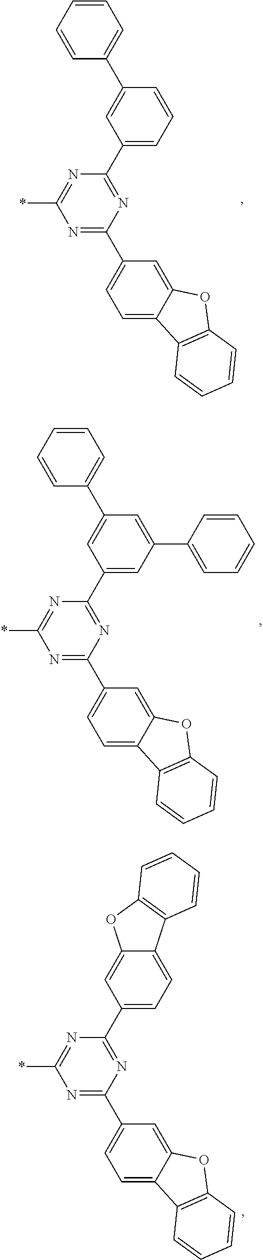

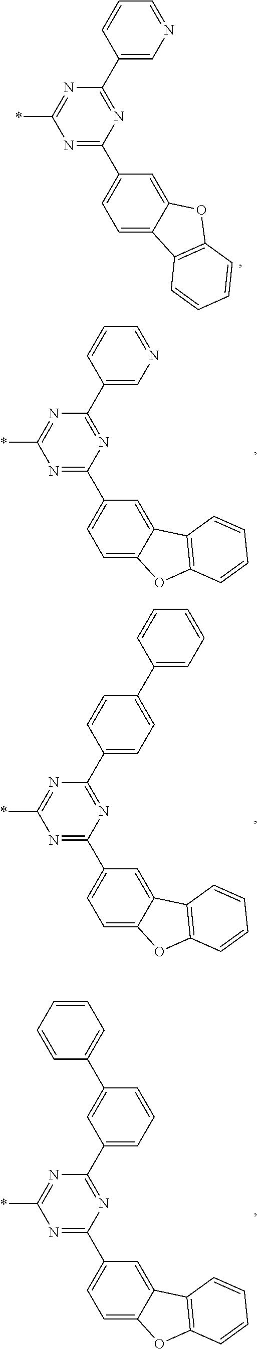

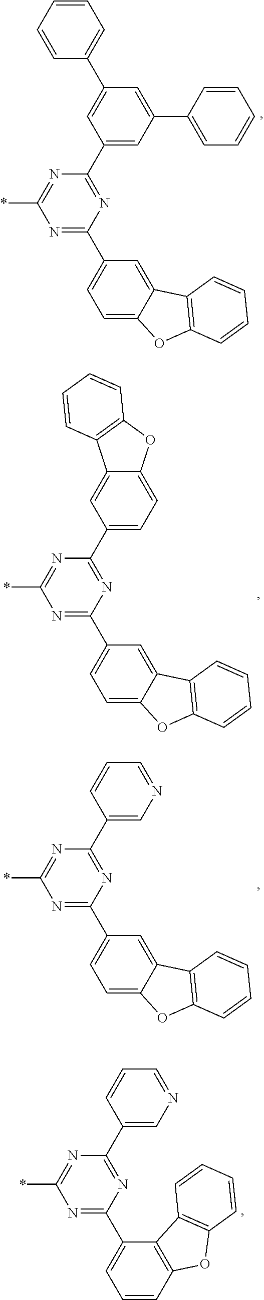

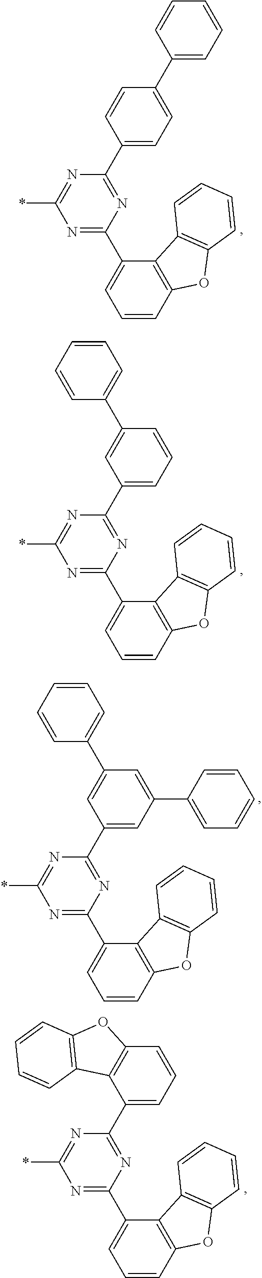

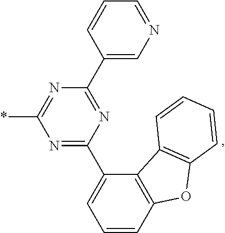 [SMILES: *c1nc(-c2cccnc2)nc(-c2cccc3oc4ccccc4c23)n1]

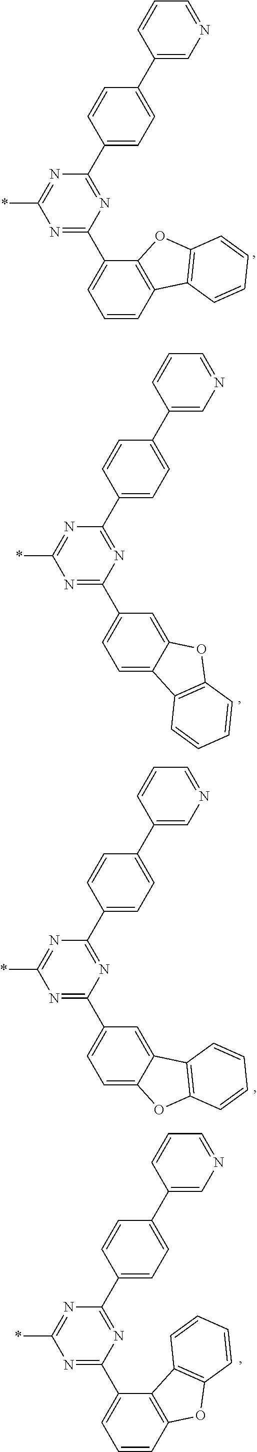

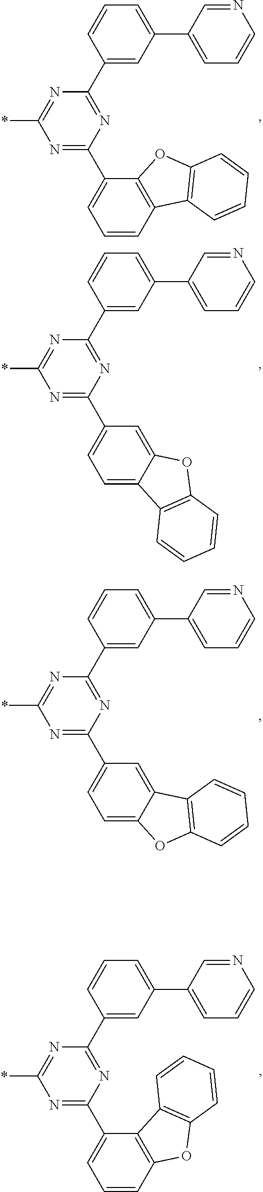

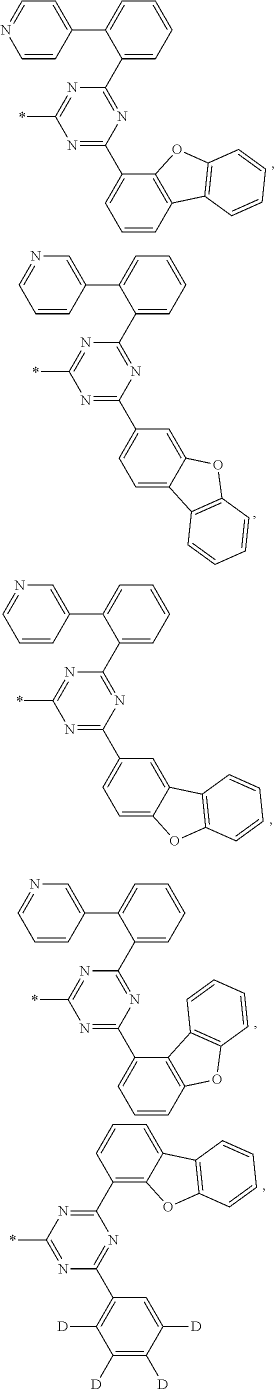

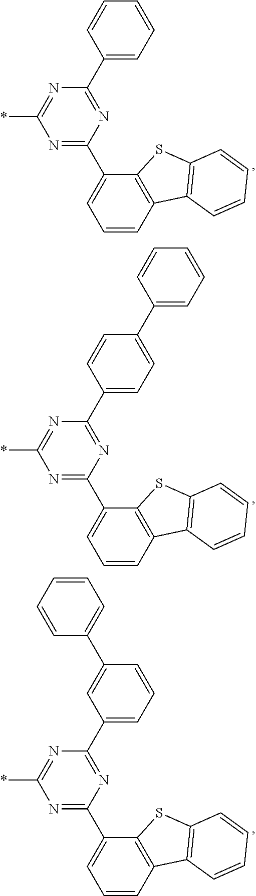

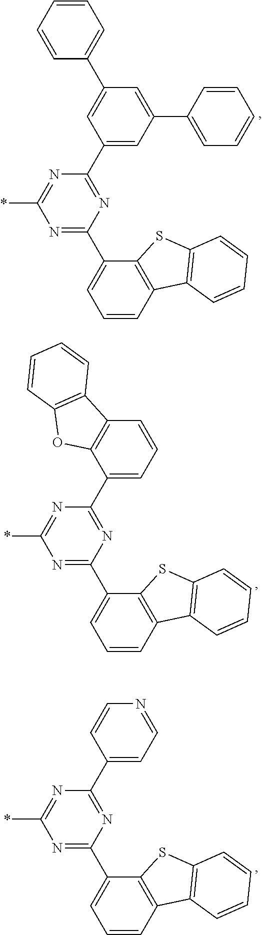

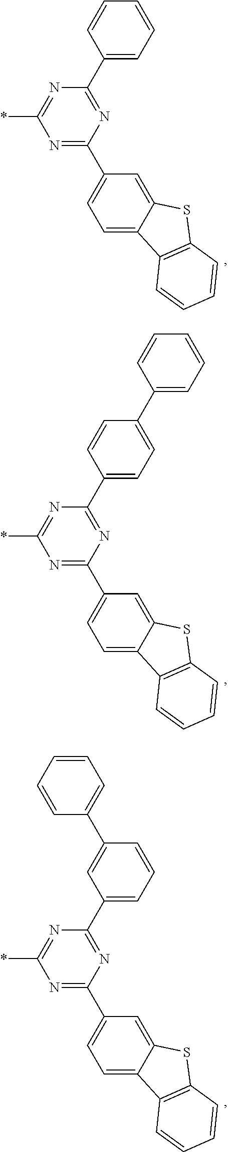

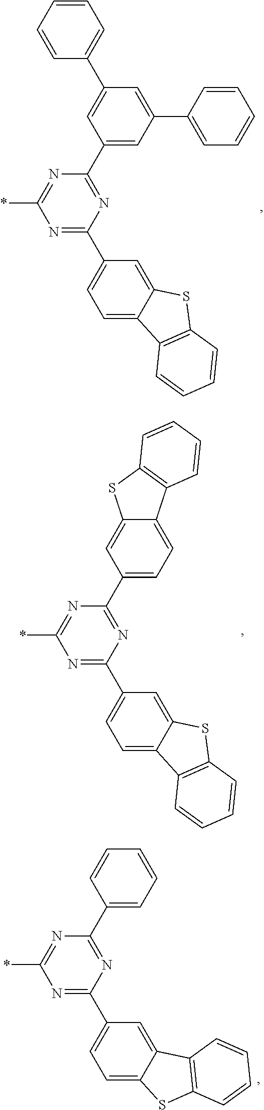

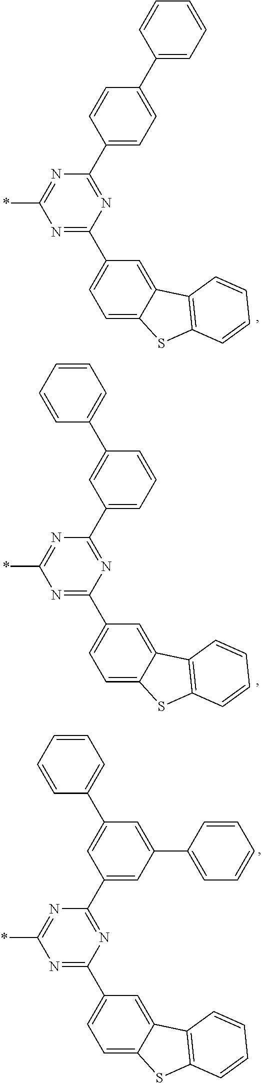 [SMILES: *c1nc(-c2ccc(-c3ccccc3)cc2)nc(-c2ccc3sc4ccccc4c3c2)n1 ; *c1nc(-c2cccc(-c3ccccc3)c2)nc(-c2ccc3sc4ccccc4c3c2)n1 ; *c1nc(-c2cc(-c3ccccc3)cc(-c3ccccc3)c2)nc(-c2ccc3sc4ccccc4c3c2)n1]

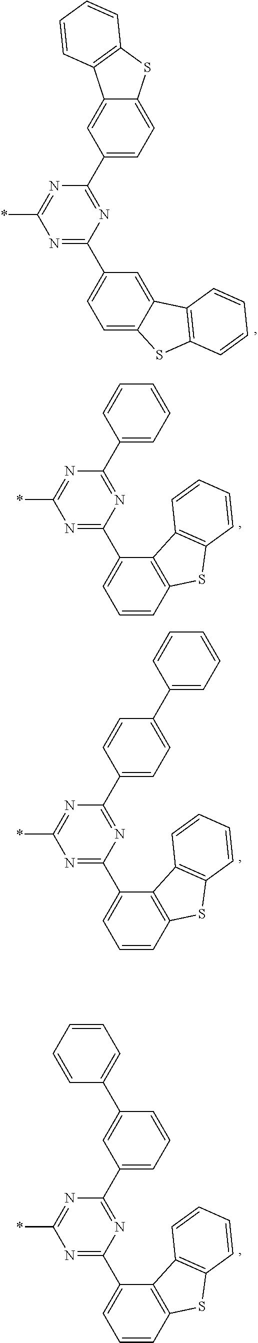

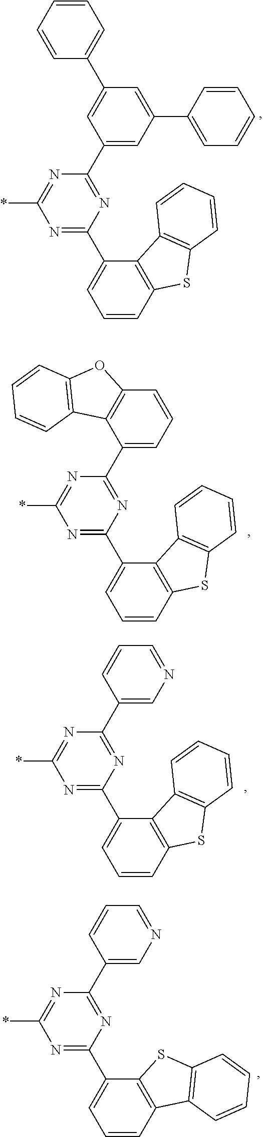

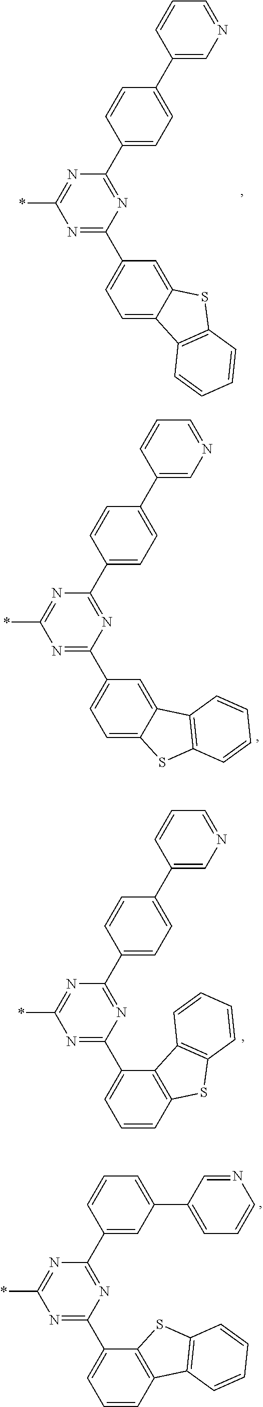

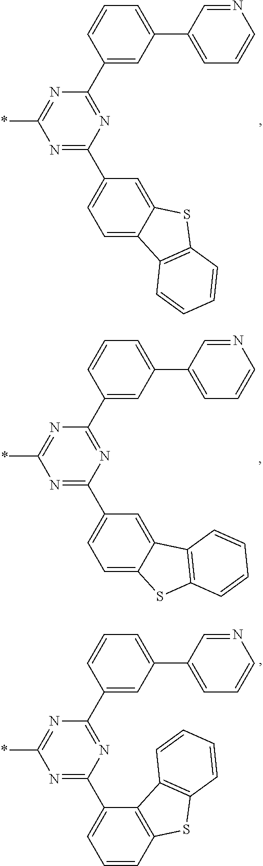

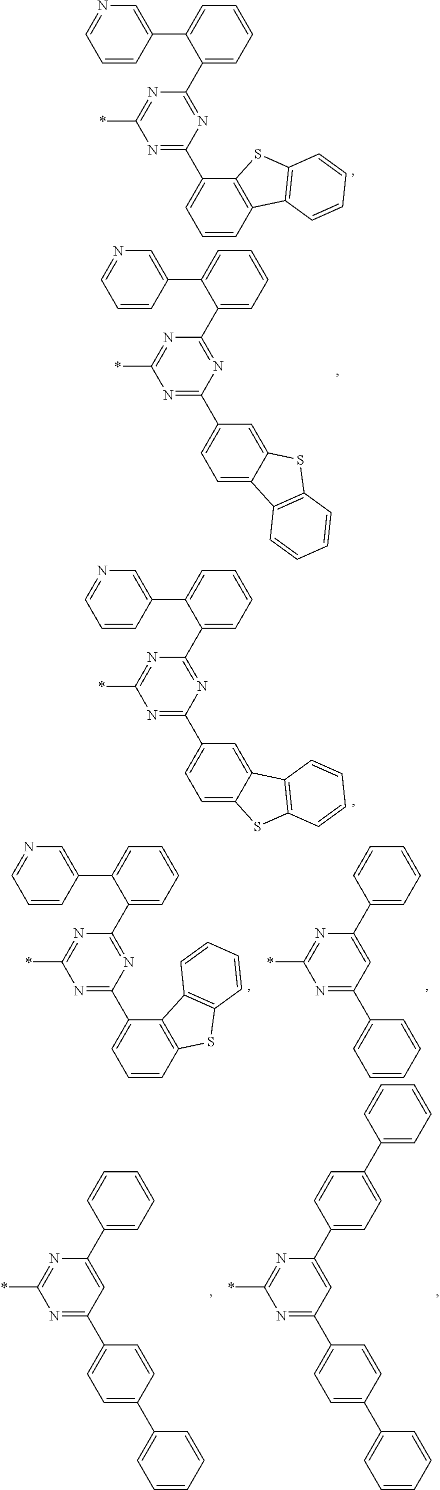

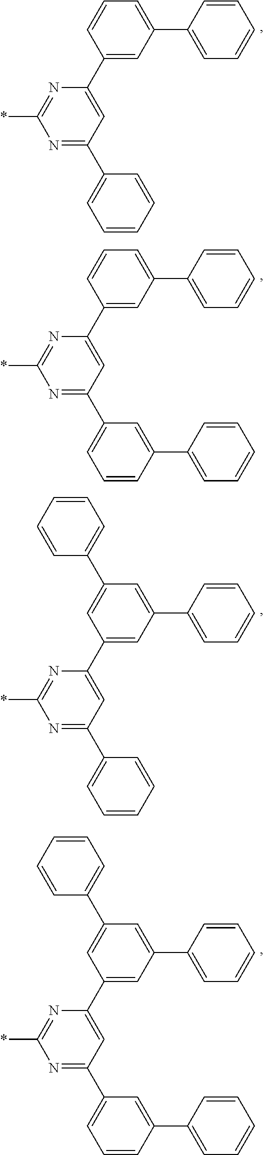

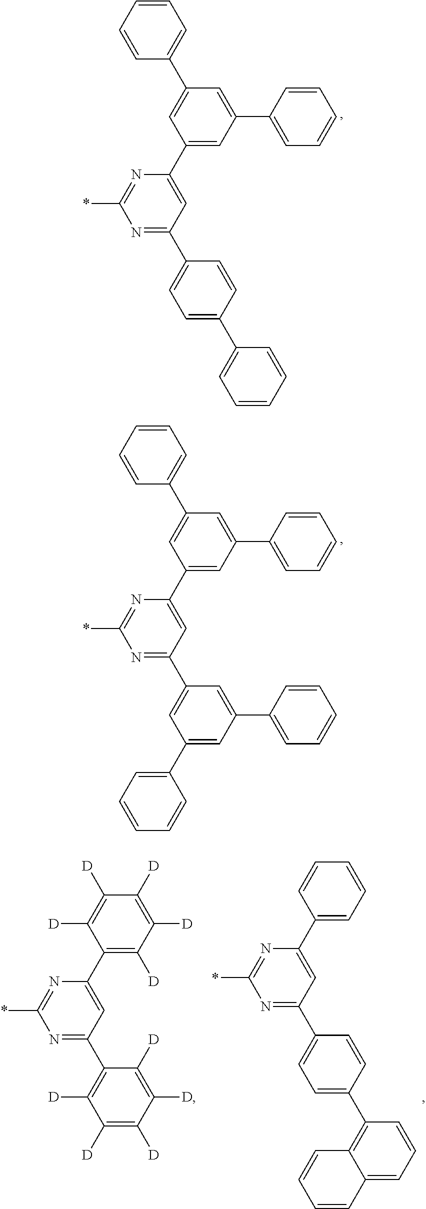

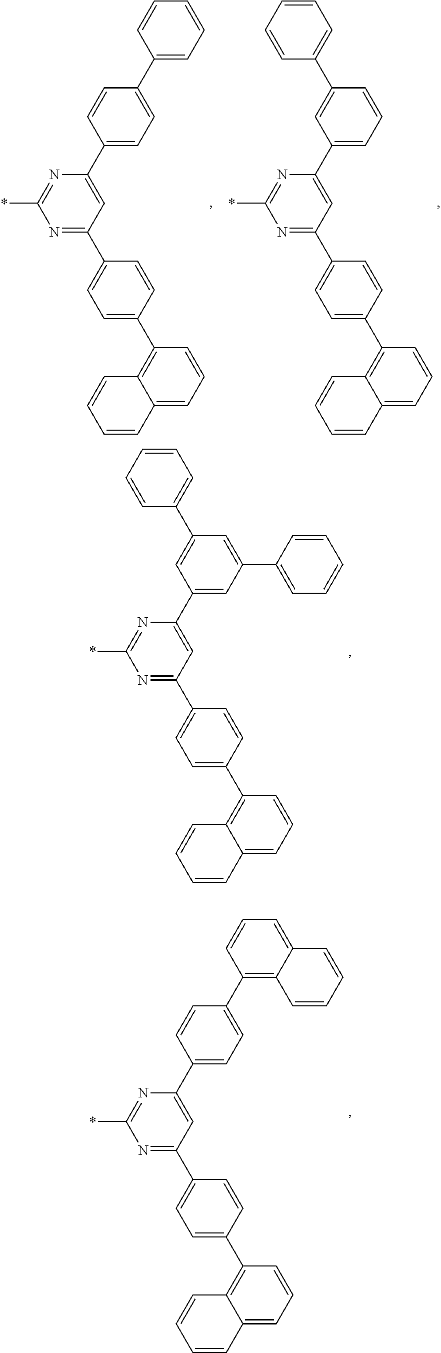

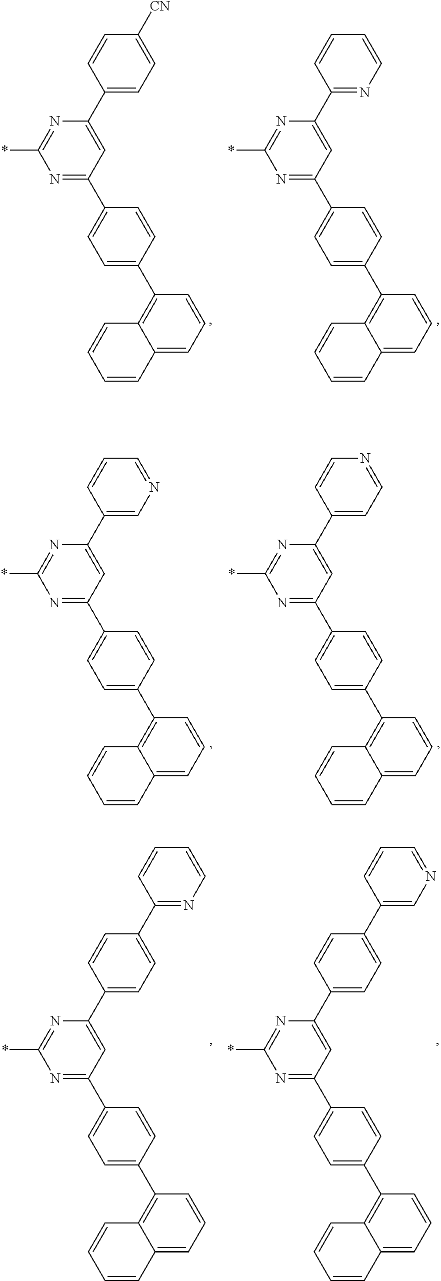

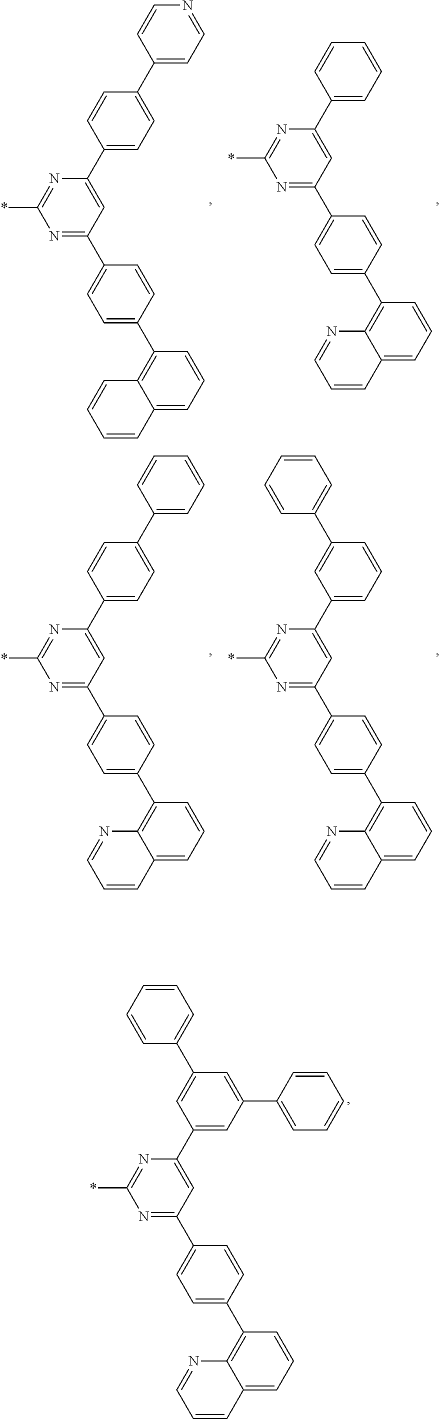

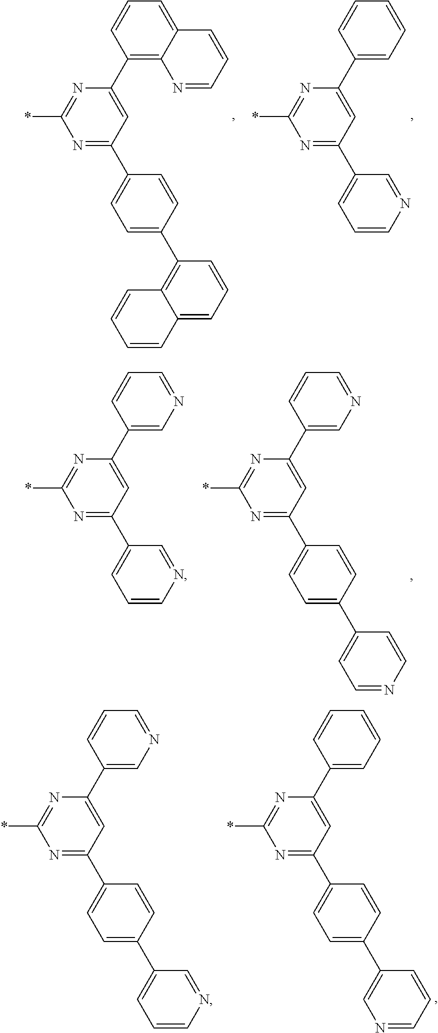

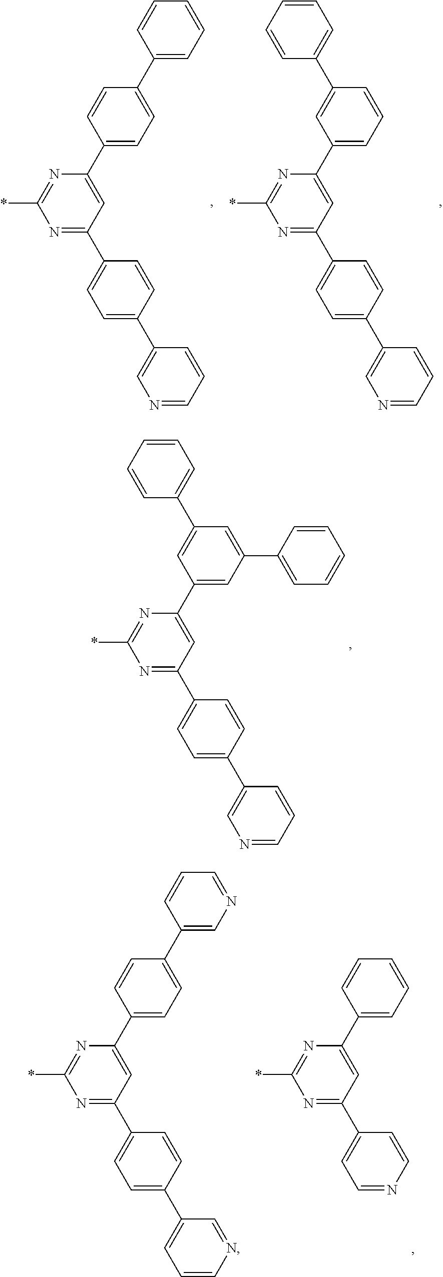

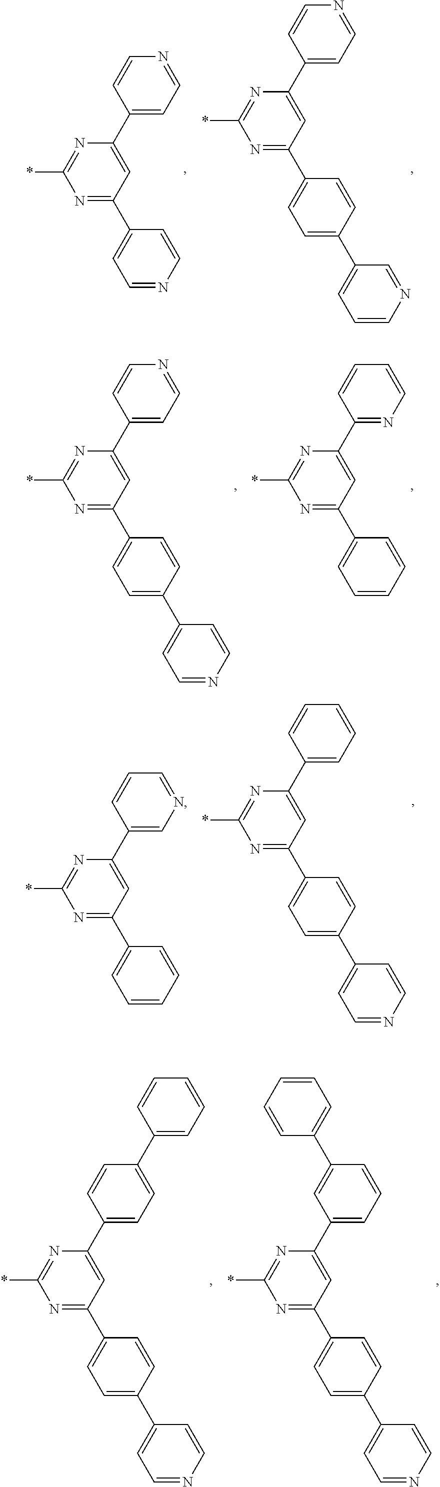

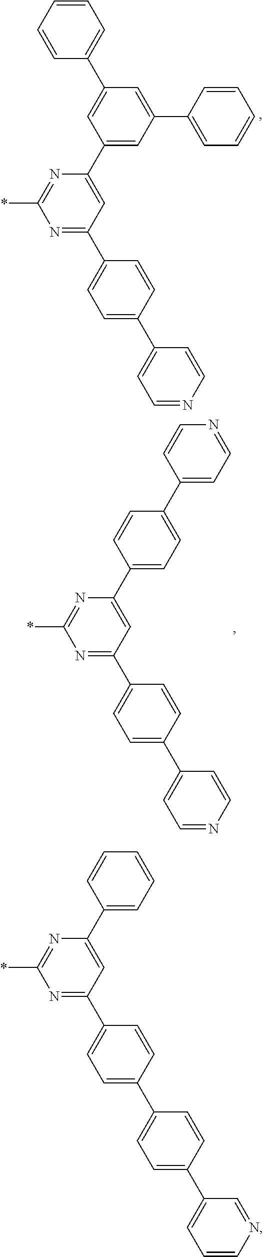

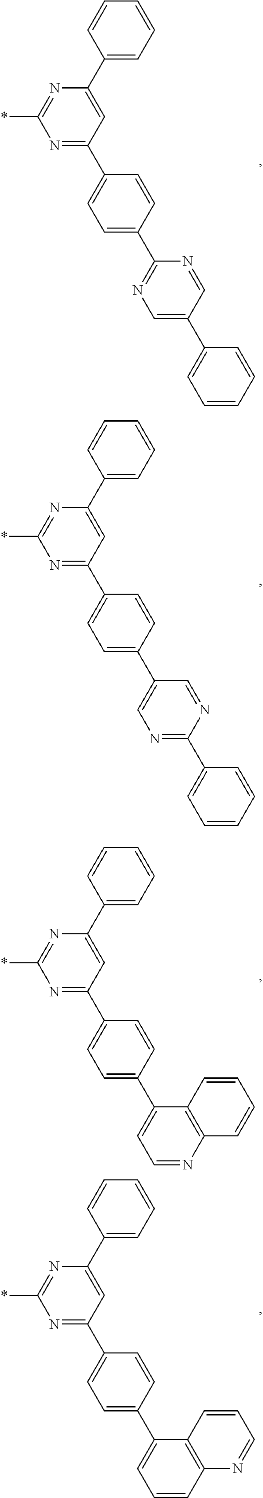

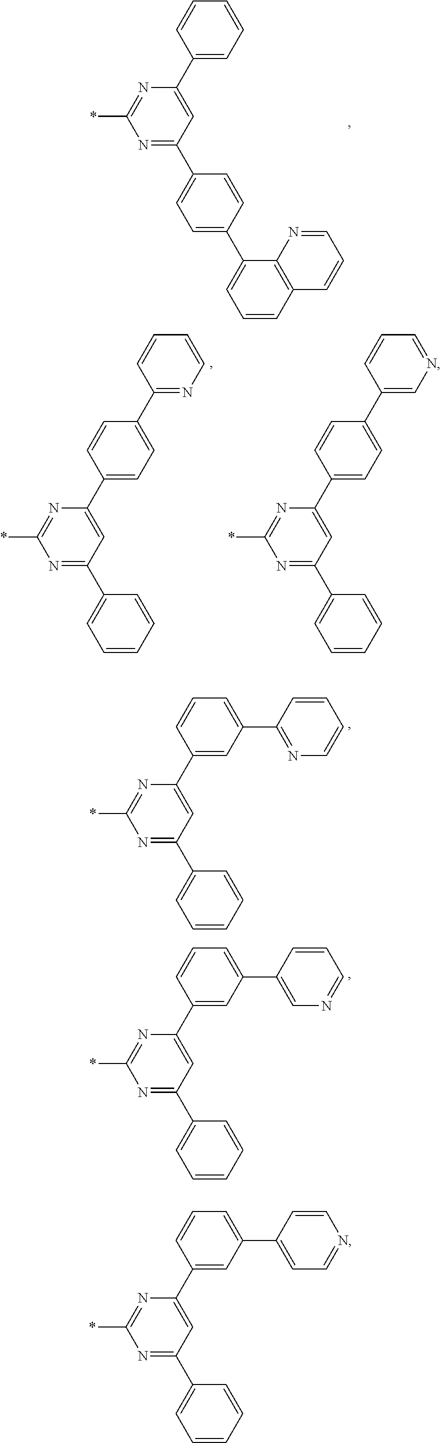

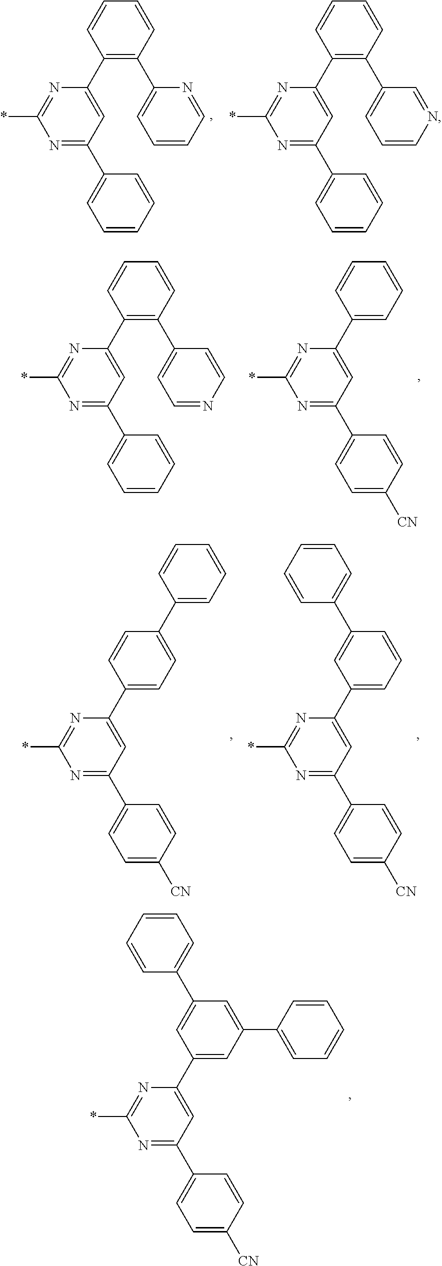

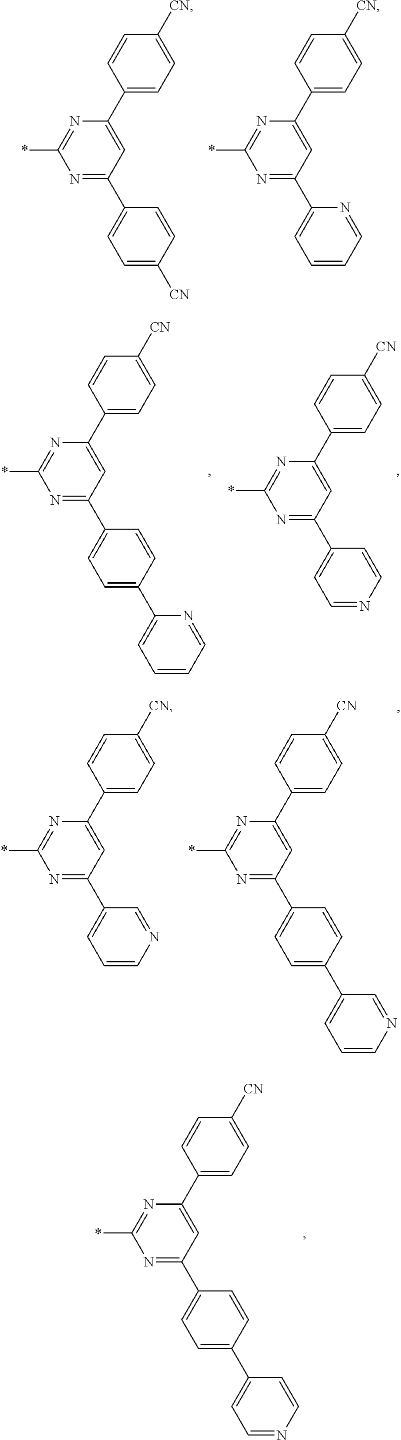

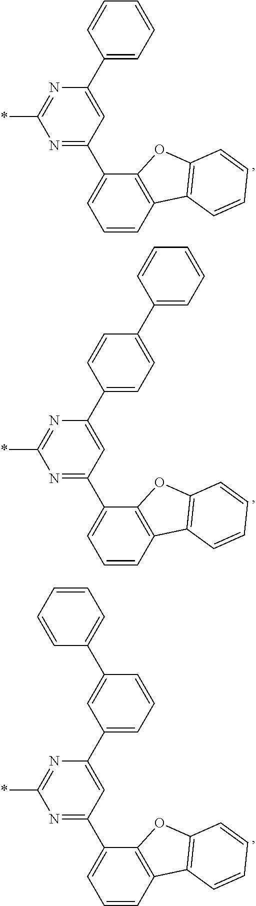

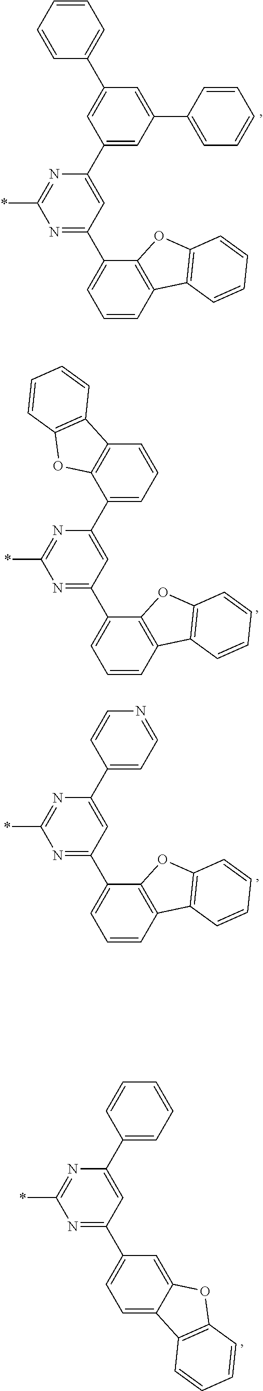

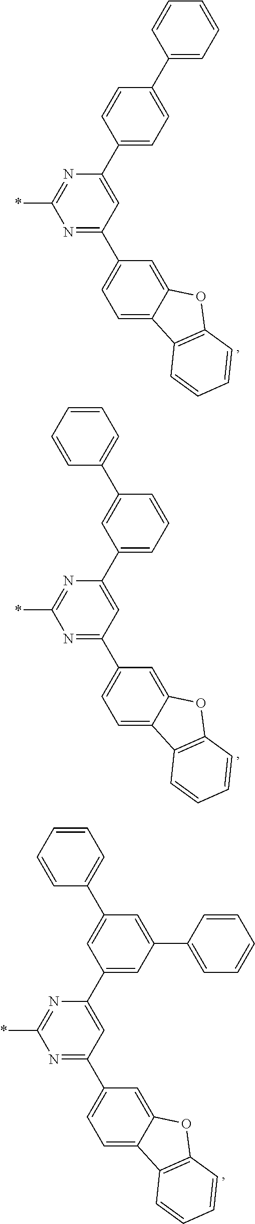 [SMILES: *c1nc(-c2ccc(-c3ccccc3)cc2)cc(-c2ccc3c(c2)oc2ccccc23)n1 ; *c1nc(-c2cccc(-c3ccccc3)c2)cc(-c2ccc3c(c2)oc2ccccc23)n1 ; *c1nc(-c2cc(-c3ccccc3)cc(-c3ccccc3)c2)cc(-c2ccc3c(c2)oc2ccccc23)n1]

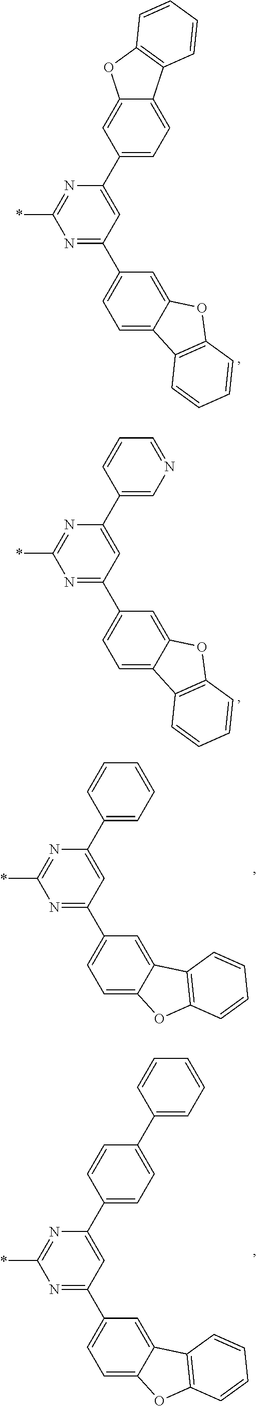

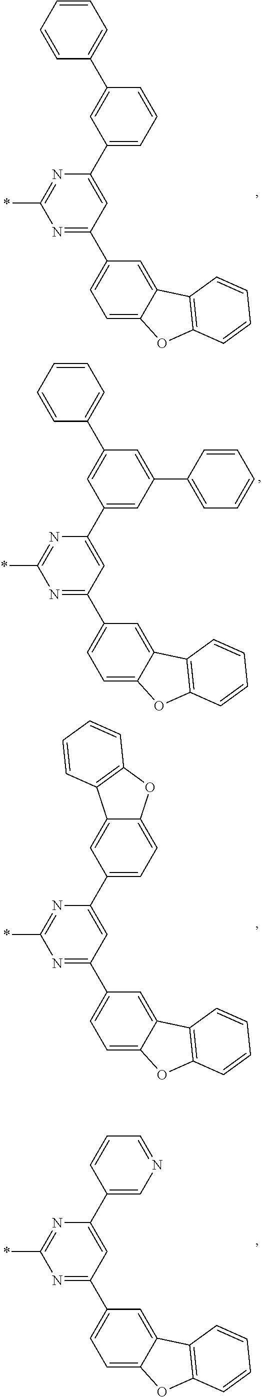

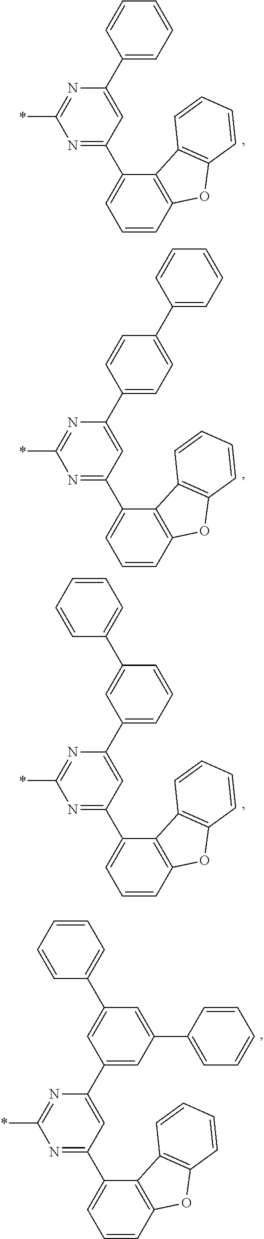

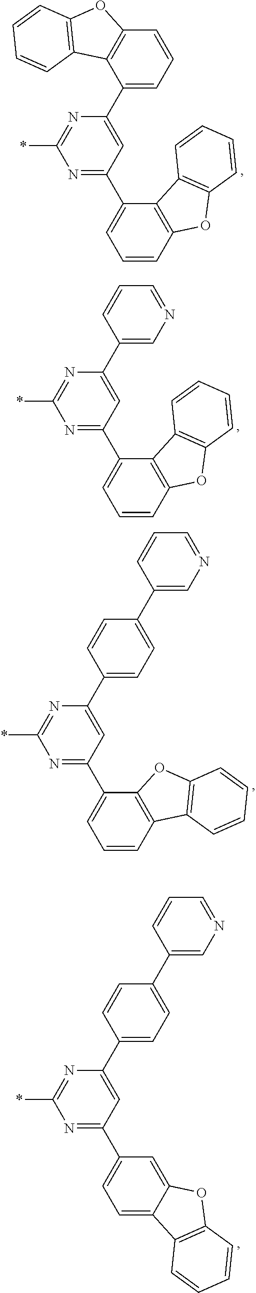

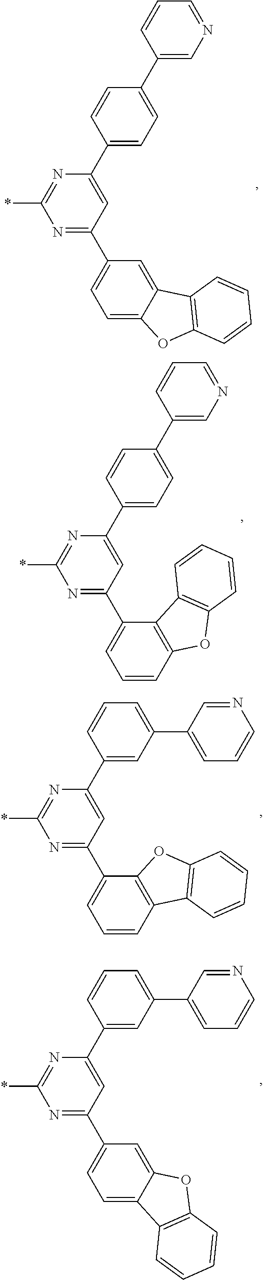

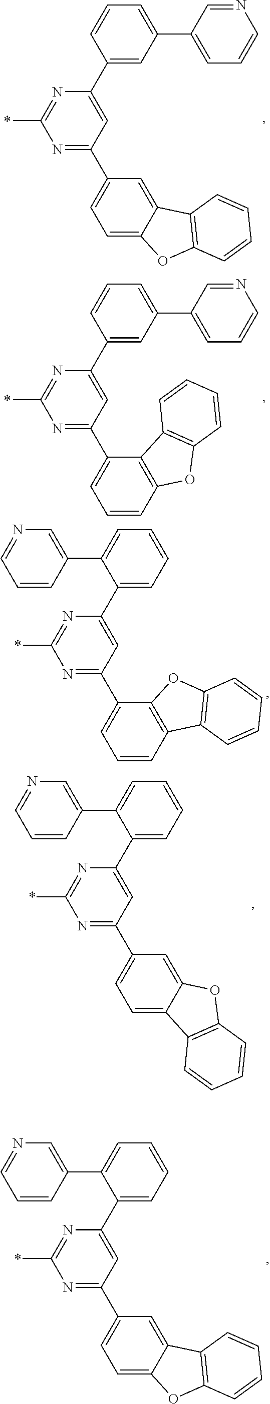

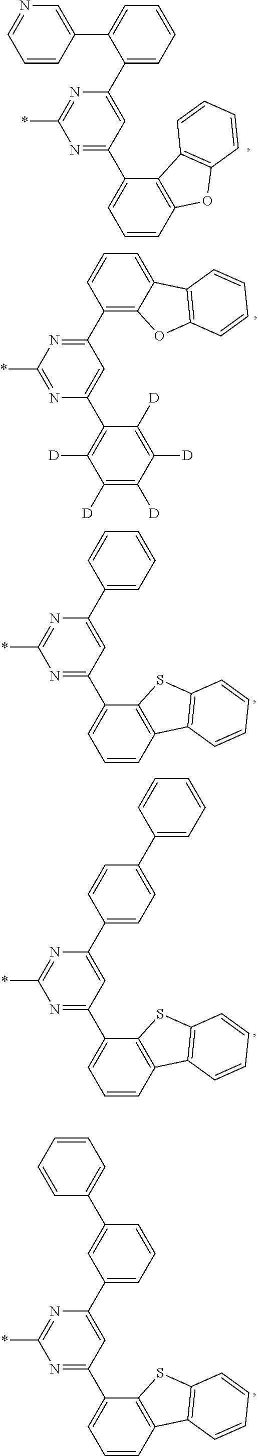

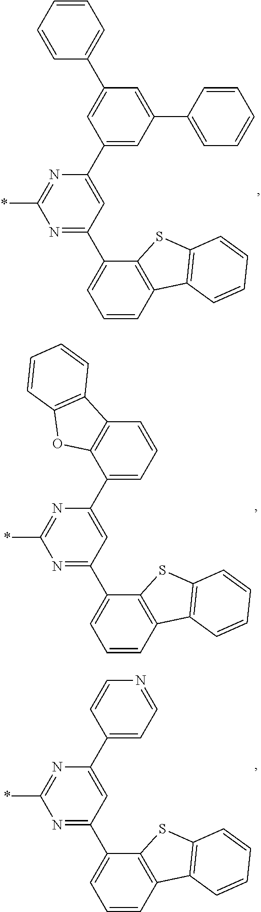

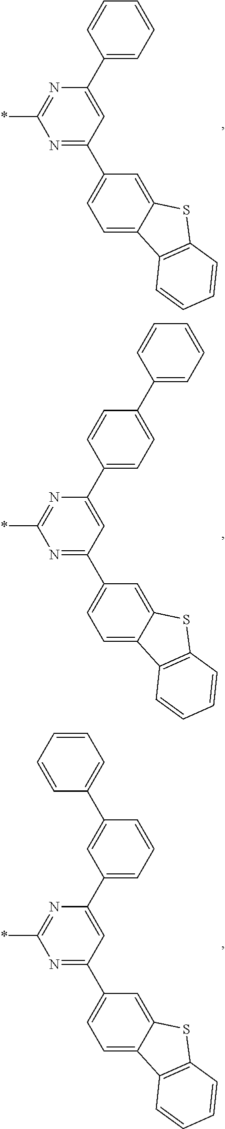

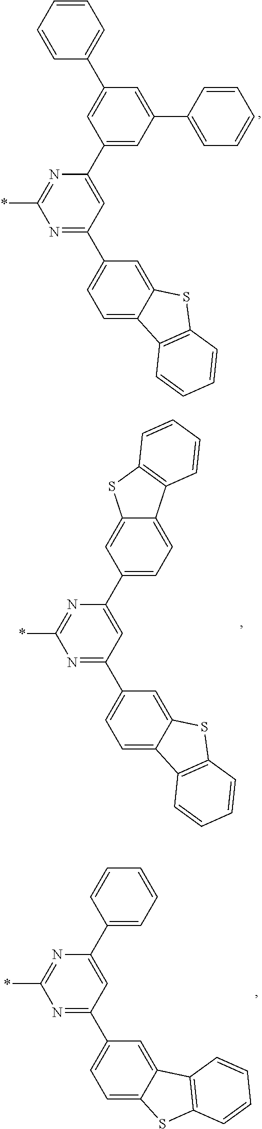

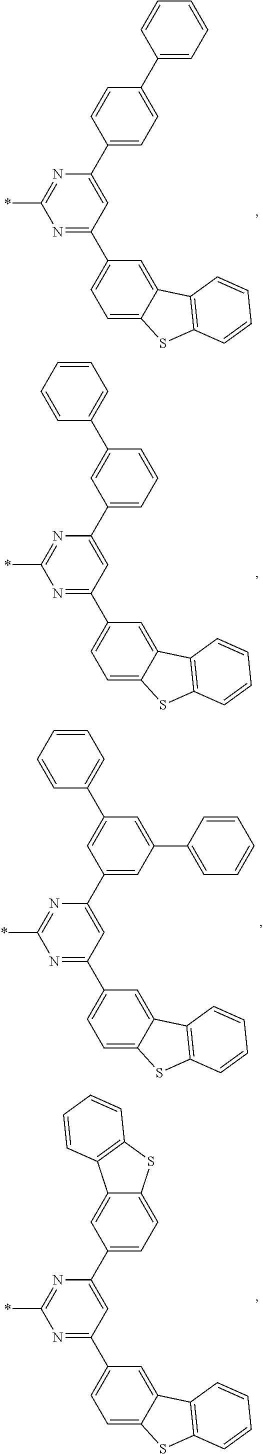 [SMILES: *c1nc(-c2ccc(-c3ccccc3)cc2)cc(-c2ccc3sc4ccccc4c3c2)n1 ; *c1nc(-c2cccc(-c3ccccc3)c2)cc(-c2ccc3sc4ccccc4c3c2)n1 ; *c1nc(-c2cc(-c3ccccc3)cc(-c3ccccc3)c2)cc(-c2ccc3sc4ccccc4c3c2)n1 ; *c1nc(-c2ccc3sc4ccccc4c3c2)cc(-c2ccc3sc4ccccc4c3c2)n1]

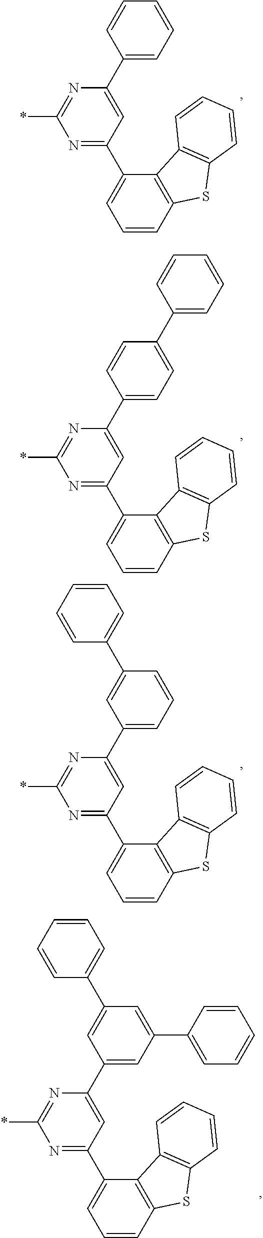

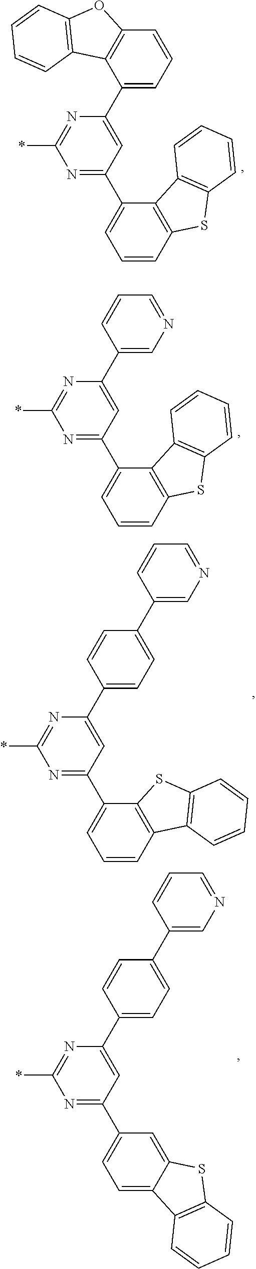

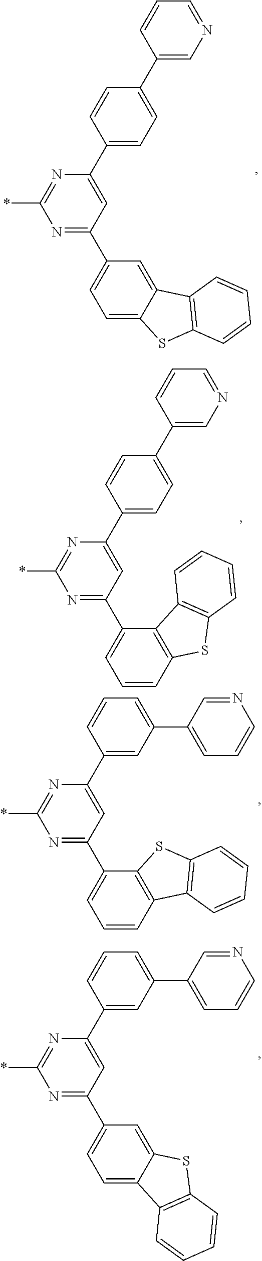

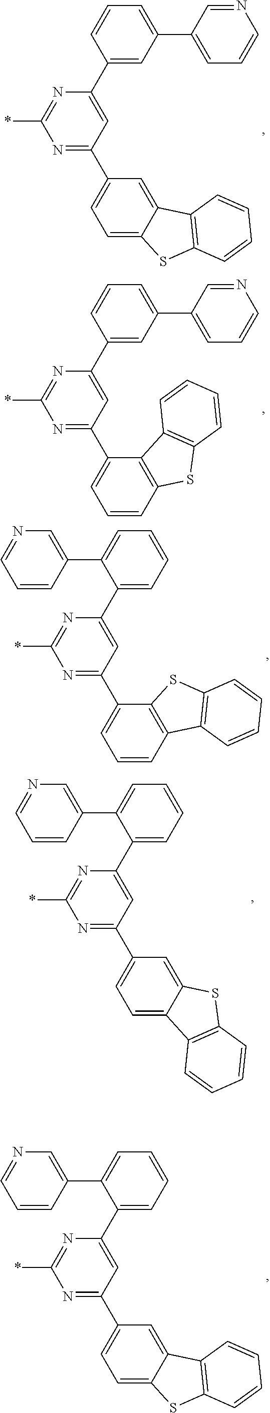

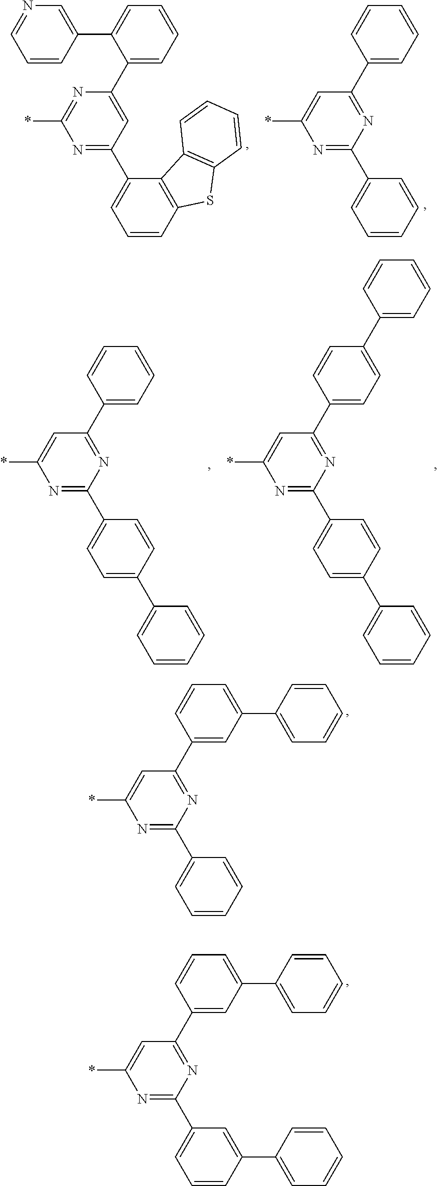

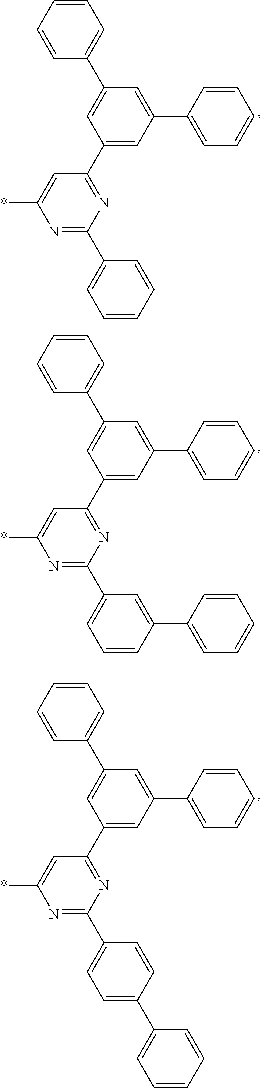

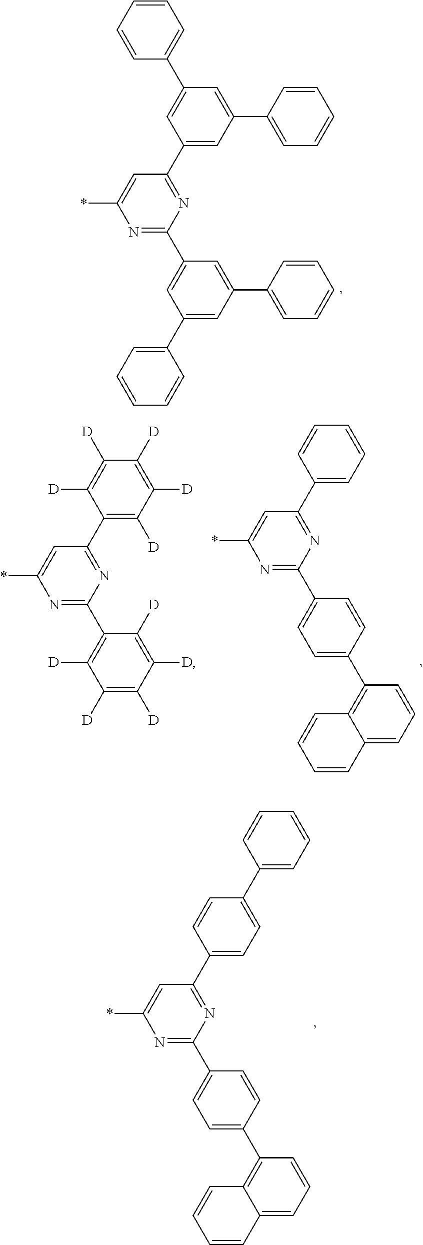

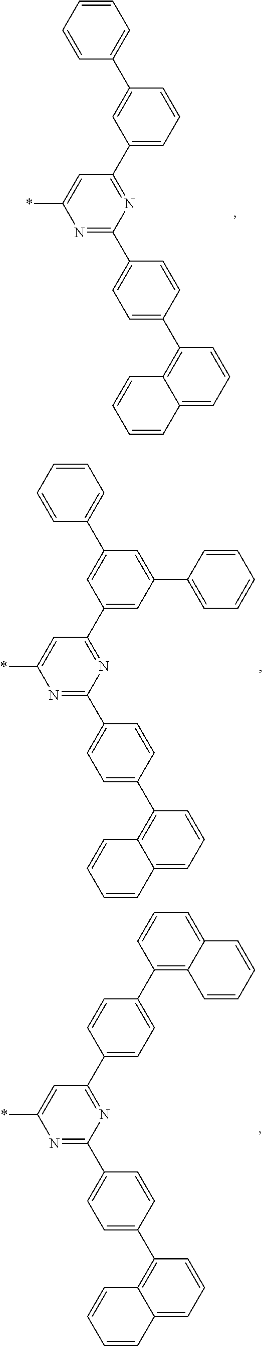

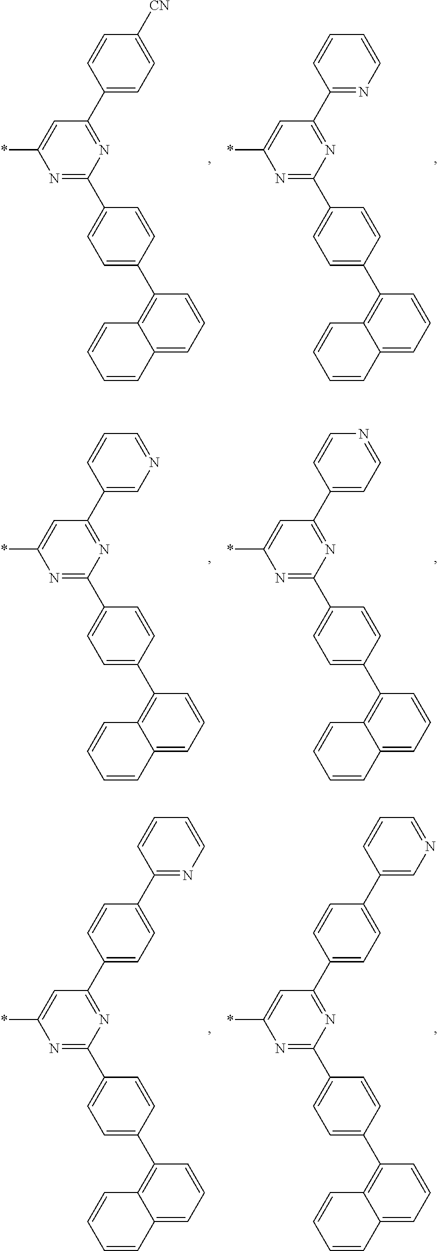

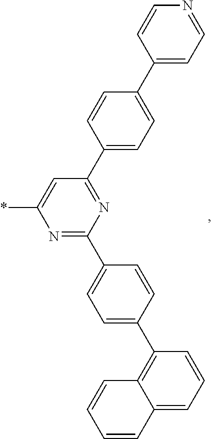 [SMILES: *c1cc(-c2ccc(-c3ccncc3)cc2)nc(-c2ccc(-c3cccc4ccccc34)cc2)n1]

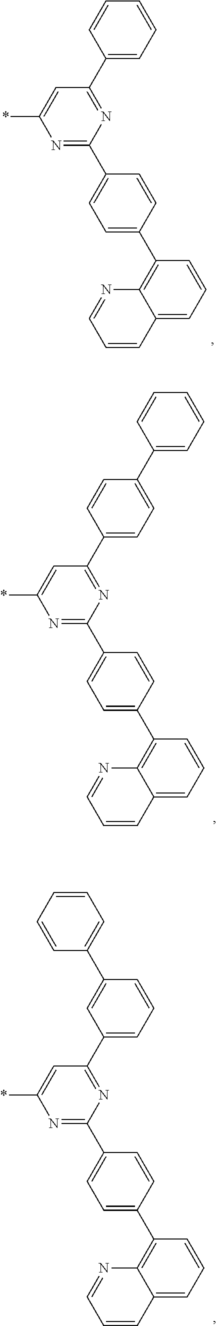 [SMILES: *c1cc(-c2ccccc2)nc(-c2ccc(-c3cccc4cccnc34)cc2)n1 ; *c1cc(-c2ccc(-c3ccccc3)cc2)nc(-c2ccc(-c3cccc4cccnc34)cc2)n1 ; *c1cc(-c2cccc(-c3ccccc3)c2)nc(-c2ccc(-c3cccc4cccnc34)cc2)n1]

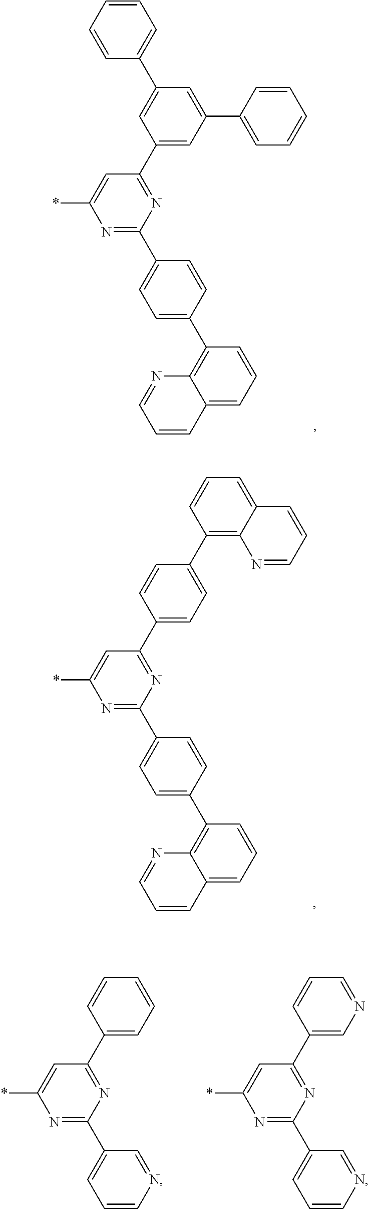 [SMILES: *c1cc(-c2cc(-c3ccccc3)cc(-c3ccccc3)c2)nc(-c2ccc(-c3cccc4cccnc34)cc2)n1 ; *c1cc(-c2ccc(-c3cccc4cccnc34)cc2)nc(-c2ccc(-c3cccc4cccnc34)cc2)n1 ; *c1cc(-c2ccccc2)nc(-c2cccnc2)n1 ; *c1cc(-c2cccnc2)nc(-c2cccnc2)n1]

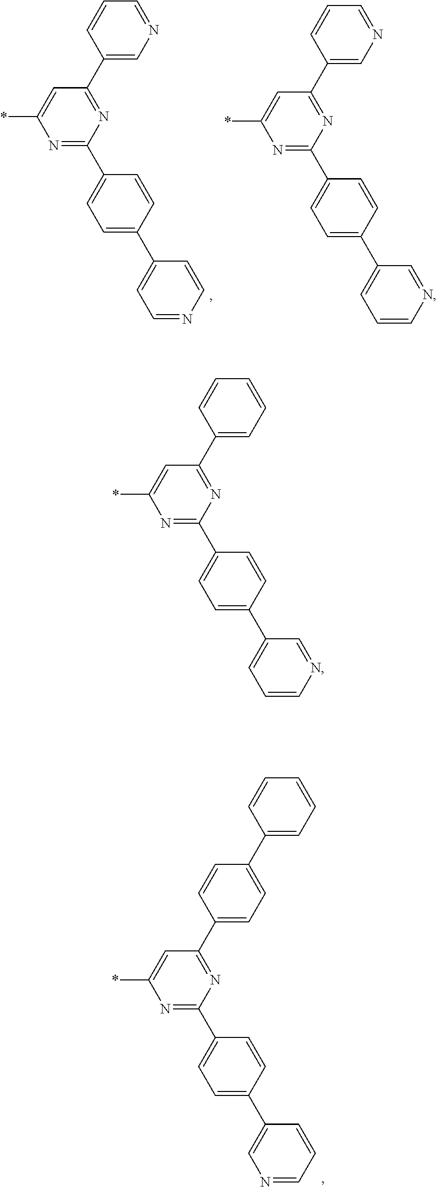 [SMILES: *c1cc(-c2cccnc2)nc(-c2ccc(-c3ccncc3)cc2)n1 ; *c1cc(-c2cccnc2)nc(-c2ccc(-c3cccnc3)cc2)n1 ; *c1cc(-c2ccccc2)nc(-c2ccc(-c3cccnc3)cc2)n1 ; *c1cc(-c2ccc(-c3ccccc3)cc2)nc(-c2ccc(-c3cccnc3)cc2)n1]

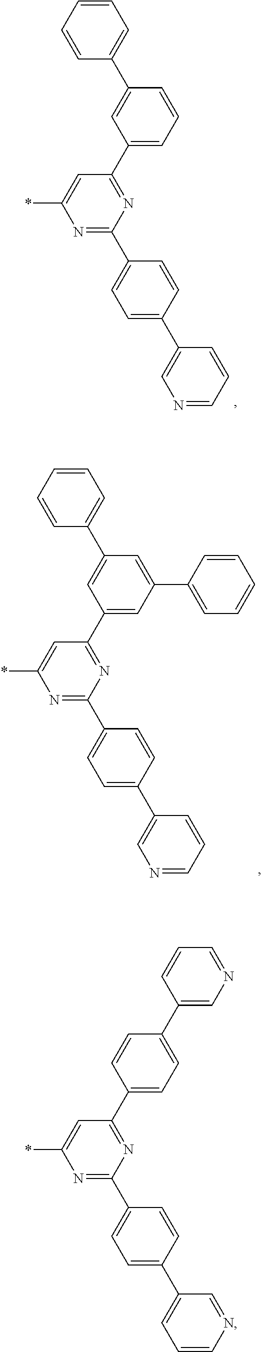 [SMILES: *c1cc(-c2cccc(-c3ccccc3)c2)nc(-c2ccc(-c3cccnc3)cc2)n1 ; *c1cc(-c2cc(-c3ccccc3)cc(-c3ccccc3)c2)nc(-c2ccc(-c3cccnc3)cc2)n1 ; *c1cc(-c2ccc(-c3cccnc3)cc2)nc(-c2ccc(-c3cccnc3)cc2)n1]

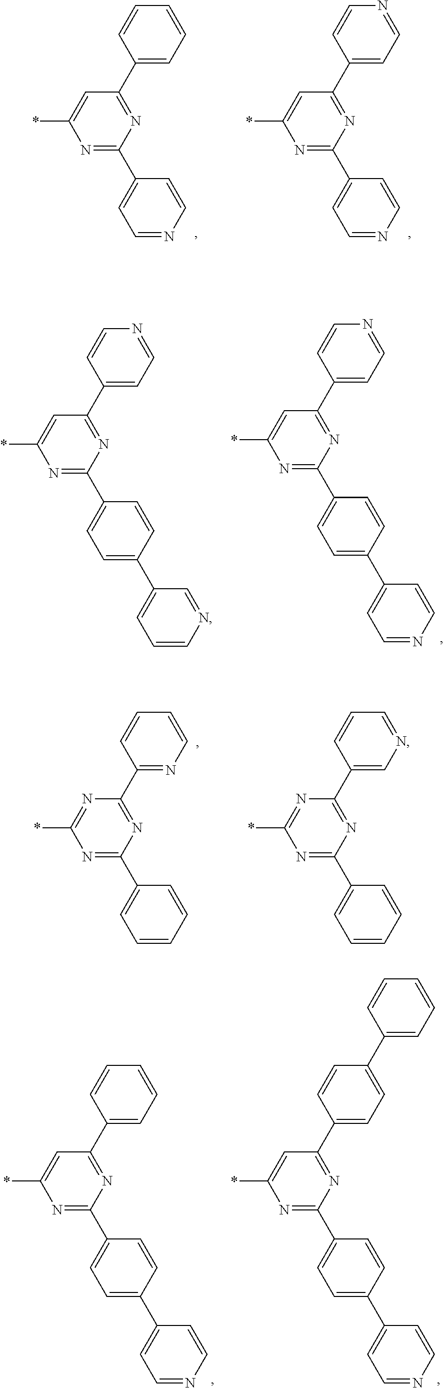

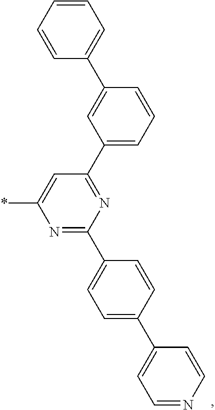 [SMILES: *c1cc(-c2cccc(-c3ccccc3)c2)nc(-c2ccc(-c3ccncc3)cc2)n1]

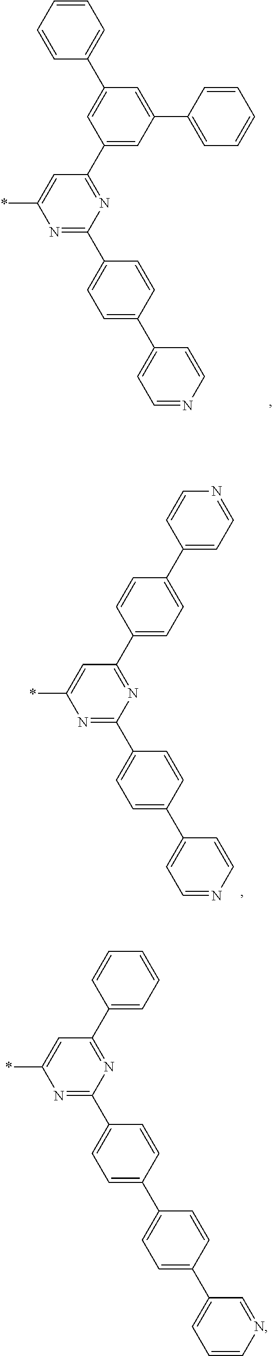 [SMILES: *c1cc(-c2cc(-c3ccccc3)cc(-c3ccccc3)c2)nc(-c2ccc(-c3ccncc3)cc2)n1 ; *c1cc(-c2ccc(-c3ccncc3)cc2)nc(-c2ccc(-c3ccncc3)cc2)n1 ; *c1cc(-c2ccccc2)nc(-c2ccc(-c3ccc(-c4cccnc4)cc3)cc2)n1]

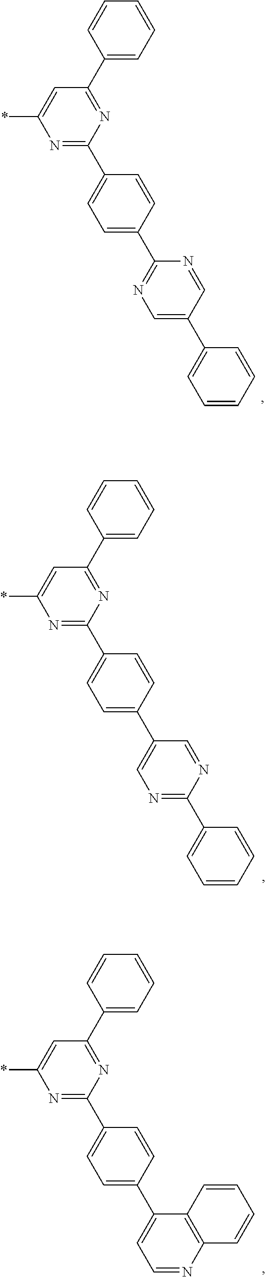 [SMILES: *c1cc(-c2ccccc2)nc(-c2ccc(-c3ncc(-c4ccccc4)cn3)cc2)n1 ; *c1cc(-c2ccccc2)nc(-c2ccc(-c3cnc(-c4ccccc4)nc3)cc2)n1 ; *c1cc(-c2ccccc2)nc(-c2ccc(-c3ccnc4ccccc34)cc2)n1]

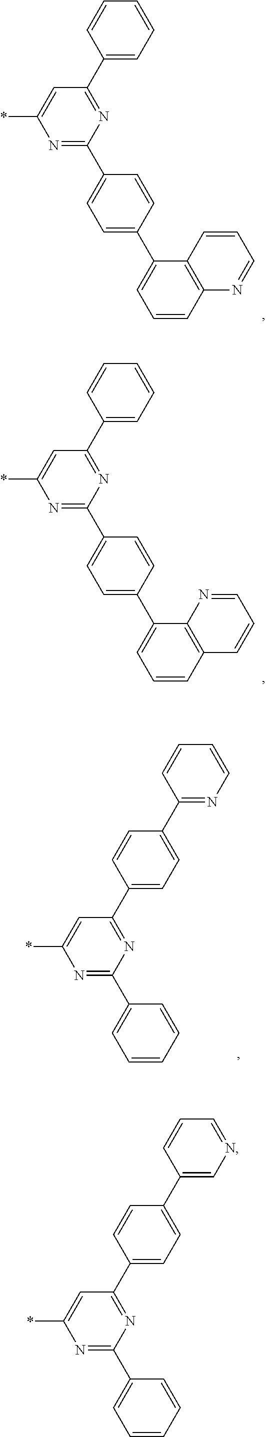 [SMILES: *c1cc(-c2ccccc2)nc(-c2ccc(-c3cccc4ncccc34)cc2)n1 ; *c1cc(-c2ccccc2)nc(-c2ccc(-c3cccc4cccnc34)cc2)n1 ; *c1cc(-c2ccc(-c3ccccn3)cc2)nc(-c2ccccc2)n1 ; *c1cc(-c2ccc(-c3cccnc3)cc2)nc(-c2ccccc2)n1]

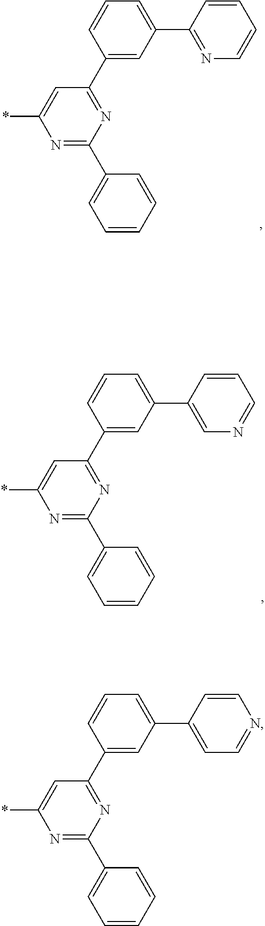 [SMILES: *c1cc(-c2cccc(-c3ccccn3)c2)nc(-c2ccccc2)n1 ; *c1cc(-c2cccc(-c3cccnc3)c2)nc(-c2ccccc2)n1 ; *c1cc(-c2cccc(-c3ccncc3)c2)nc(-c2ccccc2)n1]

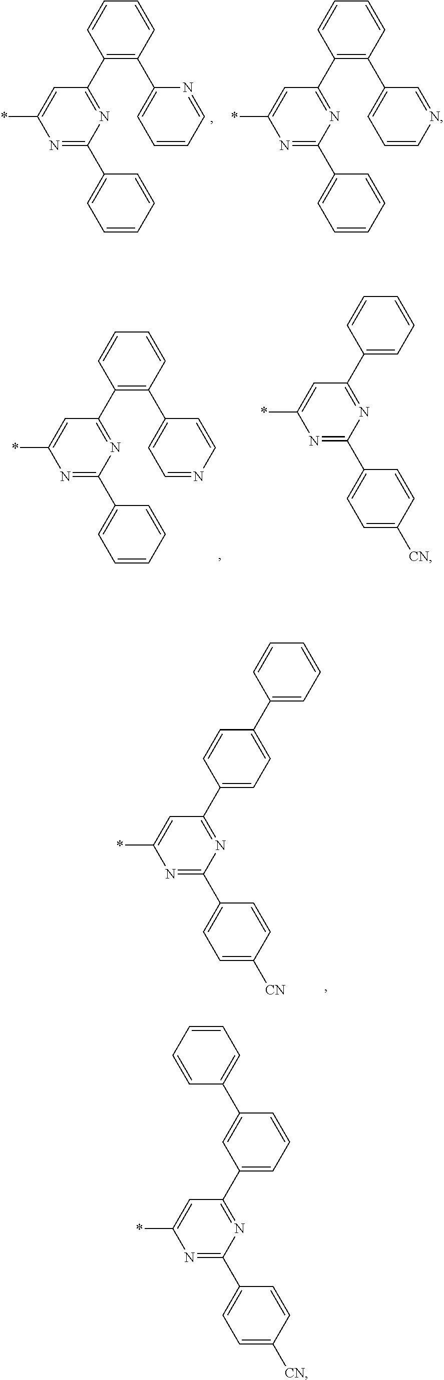

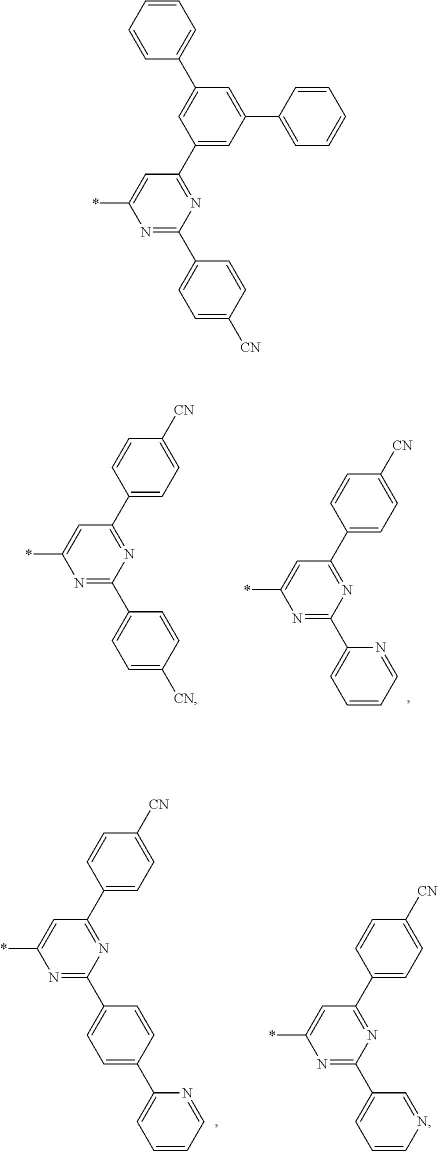 [SMILES: *c1cc(-c2cc(-c3ccccc3)cc(-c3ccccc3)c2)nc(-c2ccc(C#N)cc2)n1 ; *c1cc(-c2ccc(C#N)cc2)nc(-c2ccc(C#N)cc2)n1 ; *c1cc(-c2ccc(C#N)cc2)nc(-c2ccccn2)n1 ; *c1cc(-c2ccc(C#N)cc2)nc(-c2ccc(-c3ccccn3)cc2)n1 ; *c1cc(-c2ccc(C#N)cc2)nc(-c2cccnc2)n1]

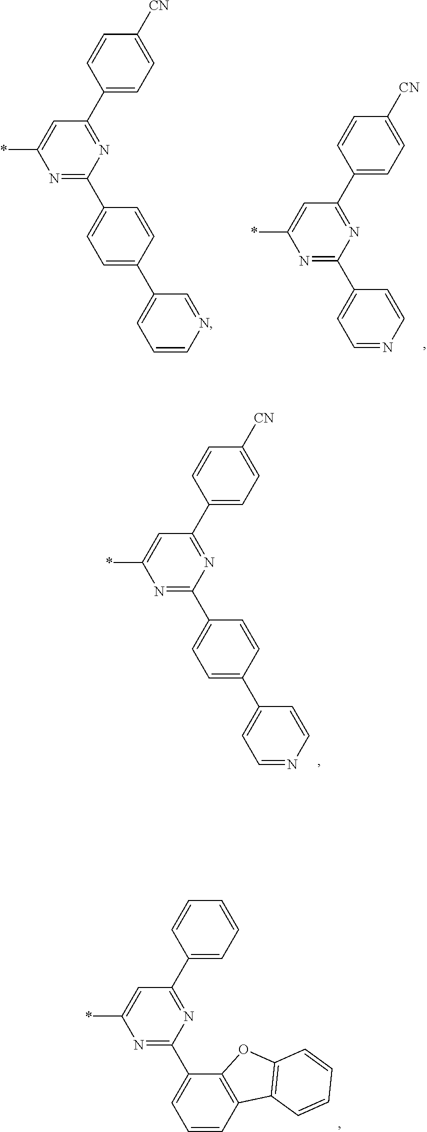 [SMILES: *c1cc(-c2ccc(C#N)cc2)nc(-c2ccc(-c3cccnc3)cc2)n1 ; *c1cc(-c2ccc(C#N)cc2)nc(-c2ccncc2)n1 ; *c1cc(-c2ccc(C#N)cc2)nc(-c2ccc(-c3ccncc3)cc2)n1 ; *c1cc(-c2ccccc2)nc(-c2cccc3c2oc2ccccc23)n1]

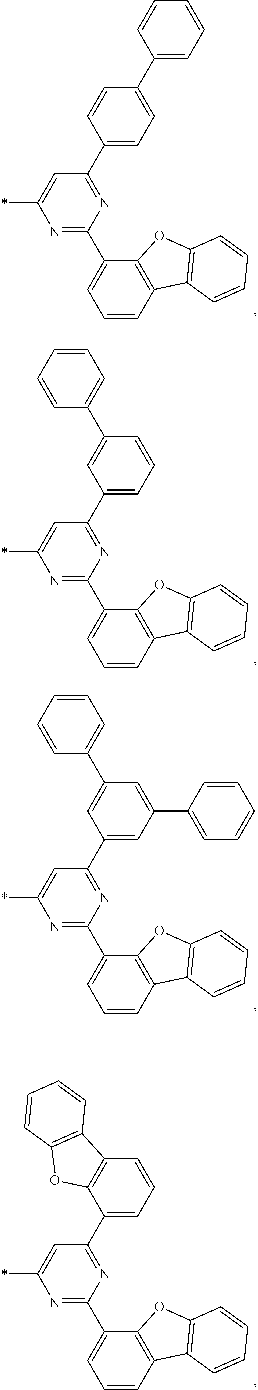

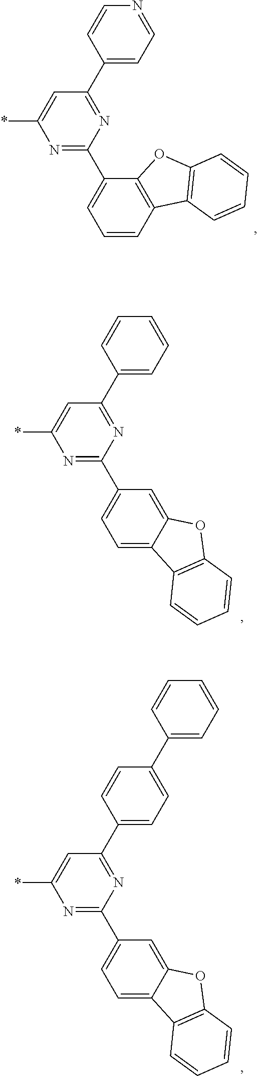 [SMILES: *c1cc(-c2ccncc2)nc(-c2cccc3c2oc2ccccc23)n1 ; *c1cc(-c2ccccc2)nc(-c2ccc3c(c2)oc2ccccc23)n1 ; *c1cc(-c2ccc(-c3ccccc3)cc2)nc(-c2ccc3c(c2)oc2ccccc23)n1]

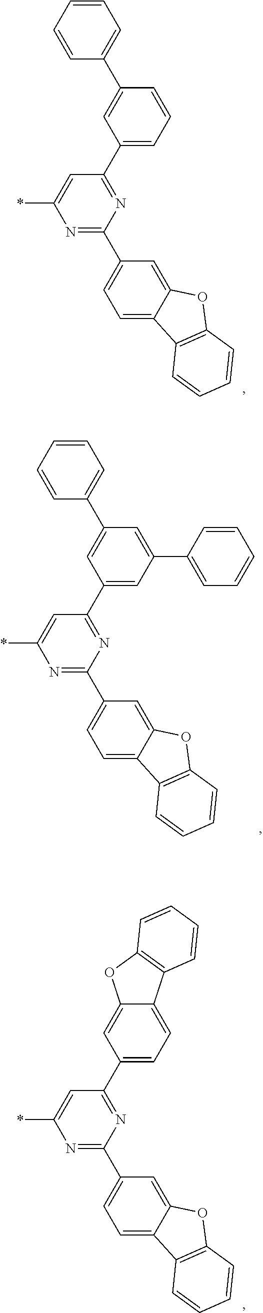 [SMILES: *c1cc(-c2cccc(-c3ccccc3)c2)nc(-c2ccc3c(c2)oc2ccccc23)n1 ; *c1cc(-c2cc(-c3ccccc3)cc(-c3ccccc3)c2)nc(-c2ccc3c(c2)oc2ccccc23)n1 ; *c1cc(-c2ccc3c(c2)oc2ccccc23)nc(-c2ccc3c(c2)oc2ccccc23)n1]

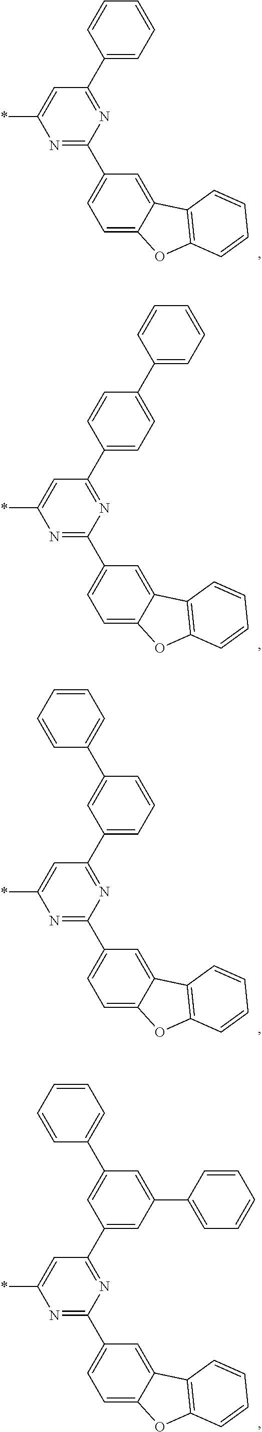 [SMILES: *c1cc(-c2ccccc2)nc(-c2ccc3oc4ccccc4c3c2)n1 ; *c1cc(-c2ccc(-c3ccccc3)cc2)nc(-c2ccc3oc4ccccc4c3c2)n1 ; *c1cc(-c2cccc(-c3ccccc3)c2)nc(-c2ccc3oc4ccccc4c3c2)n1 ; *c1cc(-c2cc(-c3ccccc3)cc(-c3ccccc3)c2)nc(-c2ccc3oc4ccccc4c3c2)n1]

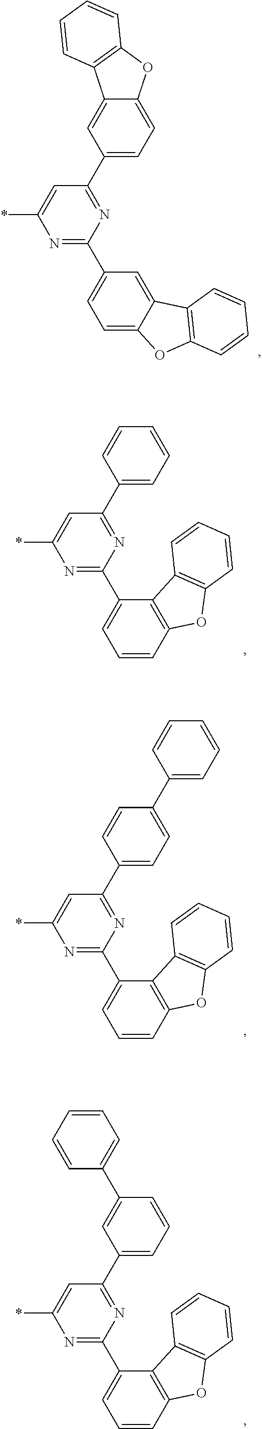 [SMILES: *c1cc(-c2ccc3oc4ccccc4c3c2)nc(-c2ccc3oc4ccccc4c3c2)n1 ; *c1cc(-c2ccccc2)nc(-c2cccc3oc4ccccc4c23)n1 ; *c1cc(-c2ccc(-c3ccccc3)cc2)nc(-c2cccc3oc4ccccc4c23)n1 ; *c1cc(-c2cccc(-c3ccccc3)c2)nc(-c2cccc3oc4ccccc4c23)n1]

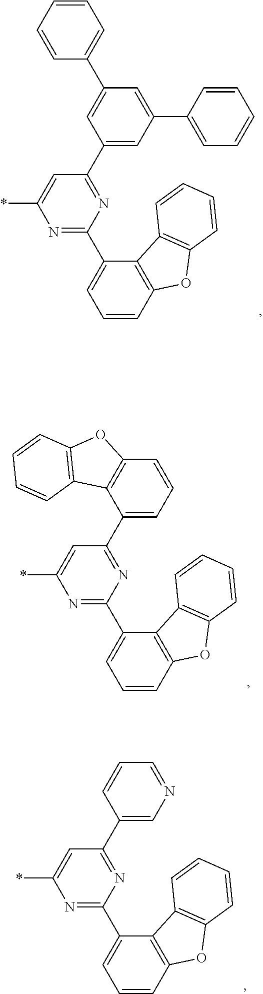 [SMILES: *c1cc(-c2cc(-c3ccccc3)cc(-c3ccccc3)c2)nc(-c2cccc3oc4ccccc4c23)n1 ; *c1cc(-c2cccc3oc4ccccc4c23)nc(-c2cccc3oc4ccccc4c23)n1 ; *c1cc(-c2cccnc2)nc(-c2cccc3oc4ccccc4c23)n1]

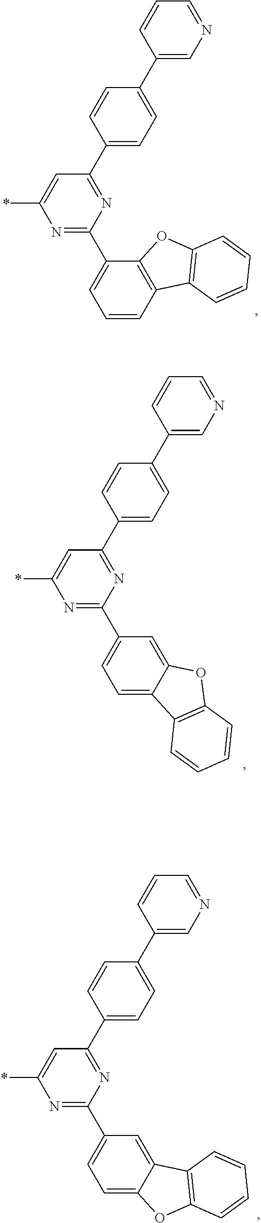 [SMILES: *c1cc(-c2ccc(-c3cccnc3)cc2)nc(-c2cccc3c2oc2ccccc23)n1 ; *c1cc(-c2ccc(-c3cccnc3)cc2)nc(-c2ccc3c(c2)oc2ccccc23)n1 ; *c1cc(-c2ccc(-c3cccnc3)cc2)nc(-c2ccc3oc4ccccc4c3c2)n1]

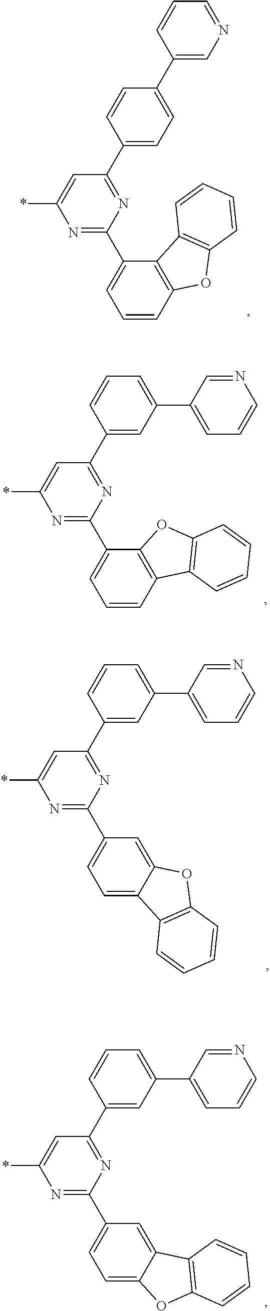 [SMILES: *c1cc(-c2ccc(-c3cccnc3)cc2)nc(-c2cccc3oc4ccccc4c23)n1 ; *c1cc(-c2cccc(-c3cccnc3)c2)nc(-c2cccc3c2oc2ccccc23)n1 ; *c1cc(-c2cccc(-c3cccnc3)c2)nc(-c2ccc3c(c2)oc2ccccc23)n1 ; *c1cc(-c2cccc(-c3cccnc3)c2)nc(-c2ccc3oc4ccccc4c3c2)n1]

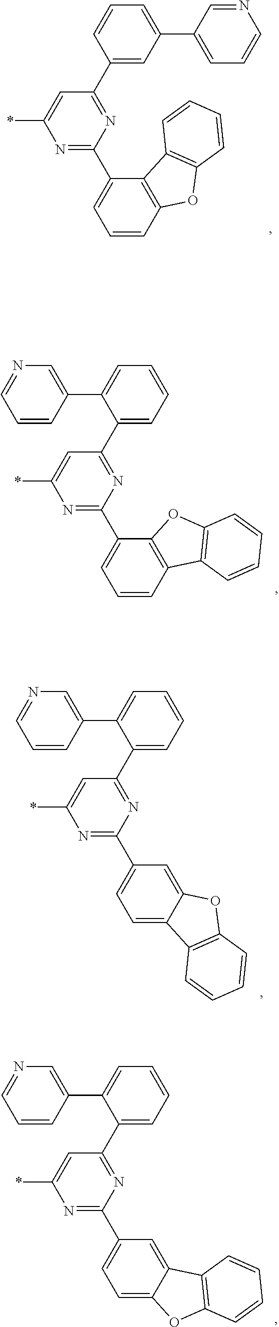 [SMILES: *c1cc(-c2cccc(-c3cccnc3)c2)nc(-c2cccc3oc4ccccc4c23)n1 ; *c1cc(-c2ccccc2-c2cccnc2)nc(-c2cccc3c2oc2ccccc23)n1 ; *c1cc(-c2ccccc2-c2cccnc2)nc(-c2ccc3c(c2)oc2ccccc23)n1 ; *c1cc(-c2ccccc2-c2cccnc2)nc(-c2ccc3oc4ccccc4c3c2)n1]

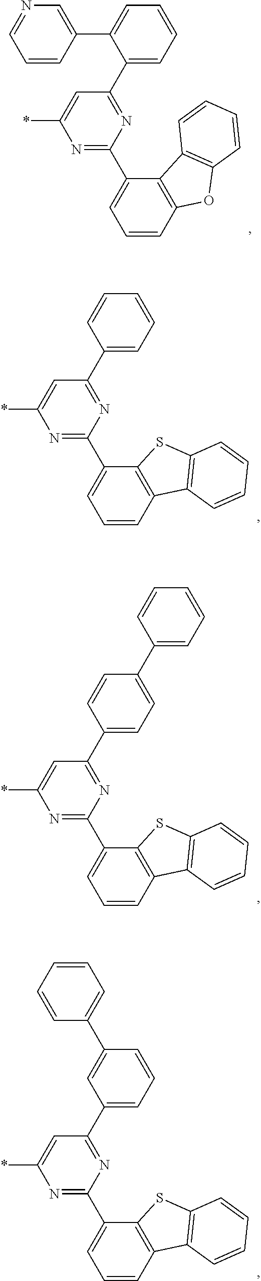 [SMILES: *c1cc(-c2ccccc2-c2cccnc2)nc(-c2cccc3oc4ccccc4c23)n1 ; *c1cc(-c2ccccc2)nc(-c2cccc3c2sc2ccccc23)n1 ; *c1cc(-c2ccc(-c3ccccc3)cc2)nc(-c2cccc3c2sc2ccccc23)n1 ; *c1cc(-c2cccc(-c3ccccc3)c2)nc(-c2cccc3c2sc2ccccc23)n1]

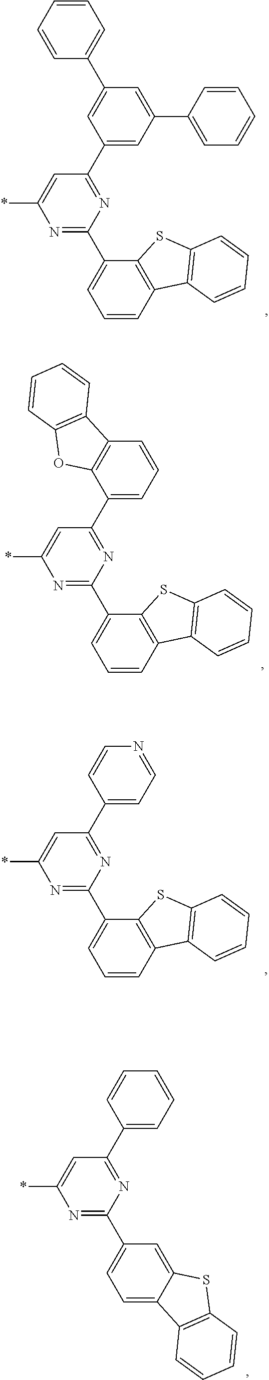 [SMILES: *c1cc(-c2cc(-c3ccccc3)cc(-c3ccccc3)c2)nc(-c2cccc3c2sc2ccccc23)n1 ; *c1cc(-c2cccc3c2oc2ccccc23)nc(-c2cccc3c2sc2ccccc23)n1 ; *c1cc(-c2ccncc2)nc(-c2cccc3c2sc2ccccc23)n1 ; *c1cc(-c2ccccc2)nc(-c2ccc3c(c2)sc2ccccc23)n1]

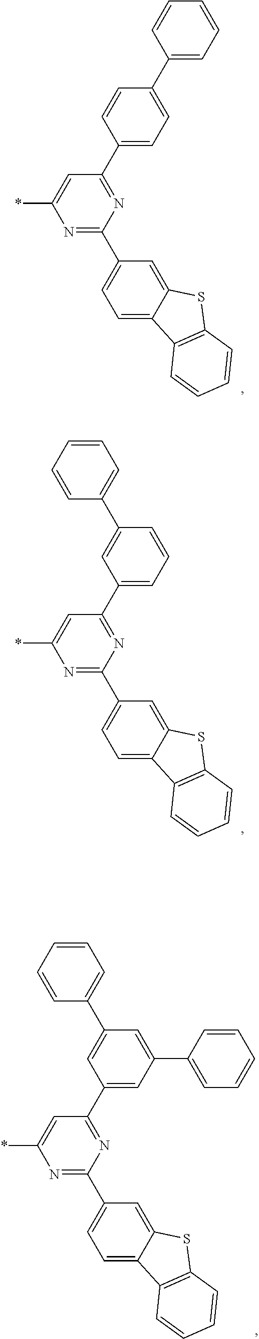 [SMILES: *c1cc(-c2ccc(-c3ccccc3)cc2)nc(-c2ccc3c(c2)sc2ccccc23)n1 ; *c1cc(-c2cccc(-c3ccccc3)c2)nc(-c2ccc3c(c2)sc2ccccc23)n1 ; *c1cc(-c2cc(-c3ccccc3)cc(-c3ccccc3)c2)nc(-c2ccc3c(c2)sc2ccccc23)n1]

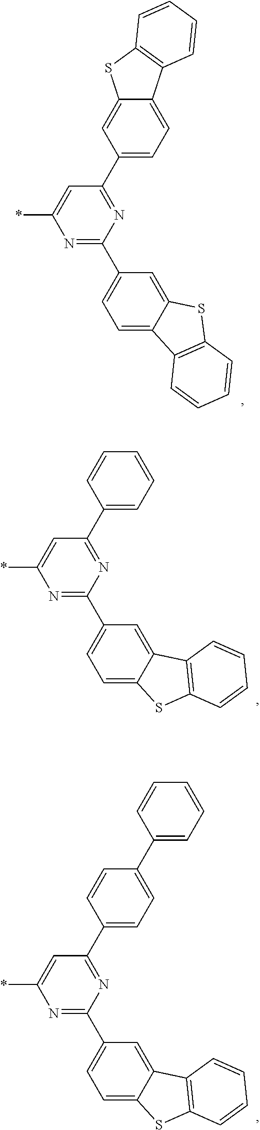 [SMILES: *c1cc(-c2ccc3c(c2)sc2ccccc23)nc(-c2ccc3c(c2)sc2ccccc23)n1 ; *c1cc(-c2ccccc2)nc(-c2ccc3sc4ccccc4c3c2)n1 ; *c1cc(-c2ccc(-c3ccccc3)cc2)nc(-c2ccc3sc4ccccc4c3c2)n1]

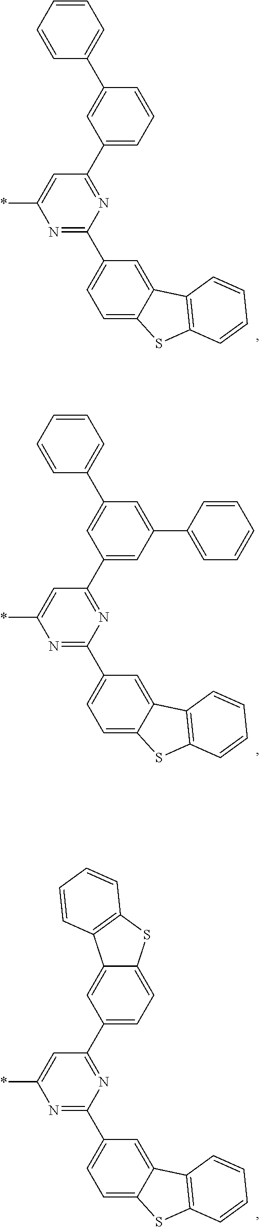 [SMILES: *c1cc(-c2cccc(-c3ccccc3)c2)nc(-c2ccc3sc4ccccc4c3c2)n1 ; *c1cc(-c2cc(-c3ccccc3)cc(-c3ccccc3)c2)nc(-c2ccc3sc4ccccc4c3c2)n1 ; *c1cc(-c2ccc3sc4ccccc4c3c2)nc(-c2ccc3sc4ccccc4c3c2)n1]

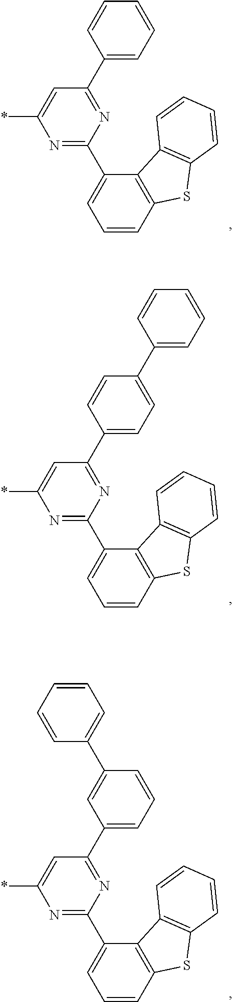 [SMILES: *c1cc(-c2ccccc2)nc(-c2cccc3sc4ccccc4c23)n1 ; *c1cc(-c2ccc(-c3ccccc3)cc2)nc(-c2cccc3sc4ccccc4c23)n1 ; *c1cc(-c2cccc(-c3ccccc3)c2)nc(-c2cccc3sc4ccccc4c23)n1]

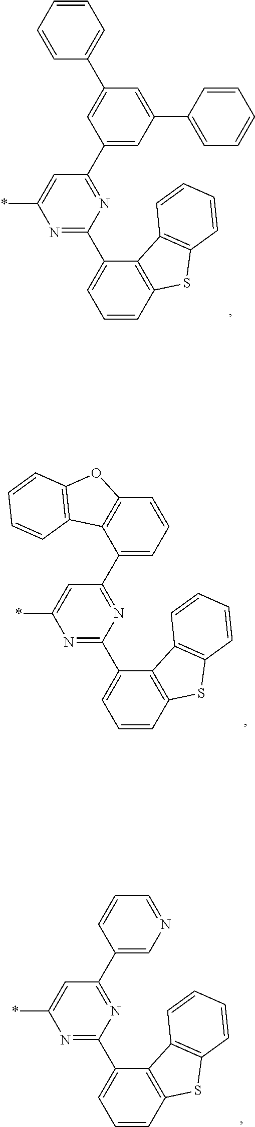 [SMILES: *c1cc(-c2cc(-c3ccccc3)cc(-c3ccccc3)c2)nc(-c2cccc3sc4ccccc4c23)n1 ; *c1cc(-c2cccc3oc4ccccc4c23)nc(-c2cccc3sc4ccccc4c23)n1 ; *c1cc(-c2cccnc2)nc(-c2cccc3sc4ccccc4c23)n1]

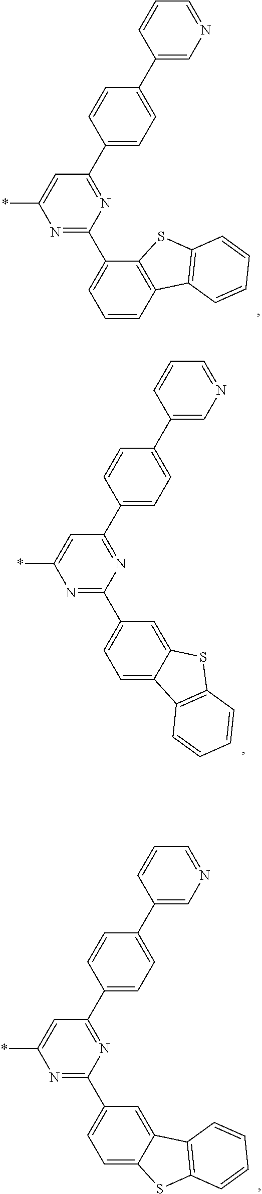 [SMILES: *c1cc(-c2ccc(-c3cccnc3)cc2)nc(-c2cccc3c2sc2ccccc23)n1 ; *c1cc(-c2ccc(-c3cccnc3)cc2)nc(-c2ccc3c(c2)sc2ccccc23)n1 ; *c1cc(-c2ccc(-c3cccnc3)cc2)nc(-c2ccc3sc4ccccc4c3c2)n1]

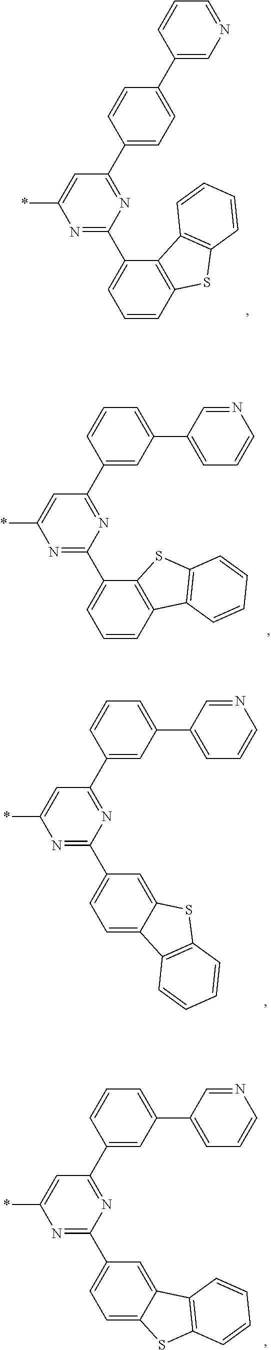 [SMILES: *c1cc(-c2ccc(-c3cccnc3)cc2)nc(-c2cccc3sc4ccccc4c23)n1 ; *c1cc(-c2cccc(-c3cccnc3)c2)nc(-c2cccc3c2sc2ccccc23)n1 ; *c1cc(-c2cccc(-c3cccnc3)c2)nc(-c2ccc3c(c2)sc2ccccc23)n1 ; *c1cc(-c2cccc(-c3cccnc3)c2)nc(-c2ccc3sc4ccccc4c3c2)n1]

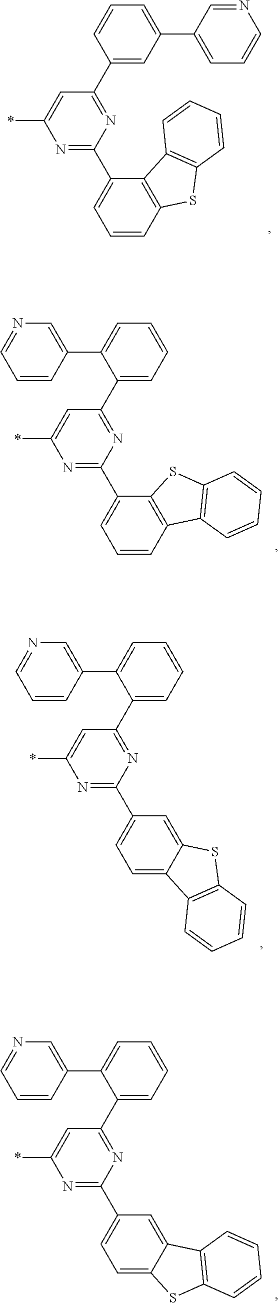 [SMILES: *c1cc(-c2cccc(-c3cccnc3)c2)nc(-c2cccc3sc4ccccc4c23)n1 ; *c1cc(-c2ccccc2-c2cccnc2)nc(-c2cccc3c2sc2ccccc23)n1 ; *c1cc(-c2ccccc2-c2cccnc2)nc(-c2ccc3c(c2)sc2ccccc23)n1 ; *c1cc(-c2ccccc2-c2cccnc2)nc(-c2ccc3sc4ccccc4c3c2)n1]

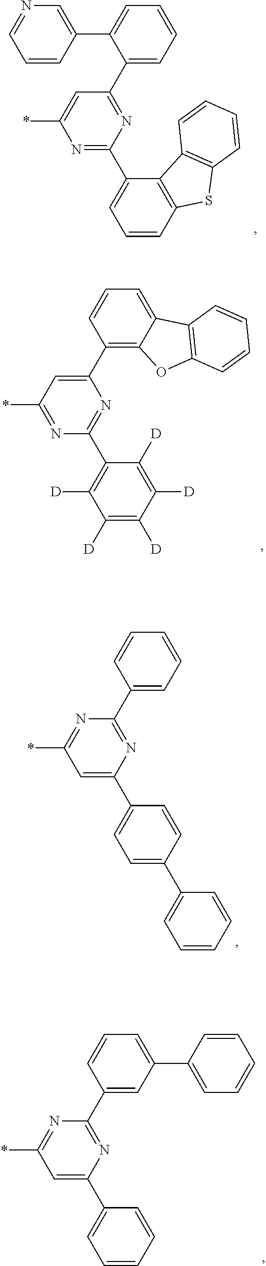 [SMILES: *c1cc(-c2ccccc2-c2cccnc2)nc(-c2cccc3sc4ccccc4c23)n1 ; *c1cc(-c2cccc3c2oc2ccccc23)nc(-c2c([2H])c([2H])c([2H])c([2H])c2[2H])n1 ; *c1cc(-c2ccc(-c3ccccc3)cc2)nc(-c2ccccc2)n1 ; *c1cc(-c2ccccc2)nc(-c2cccc(-c3ccccc3)c2)n1]

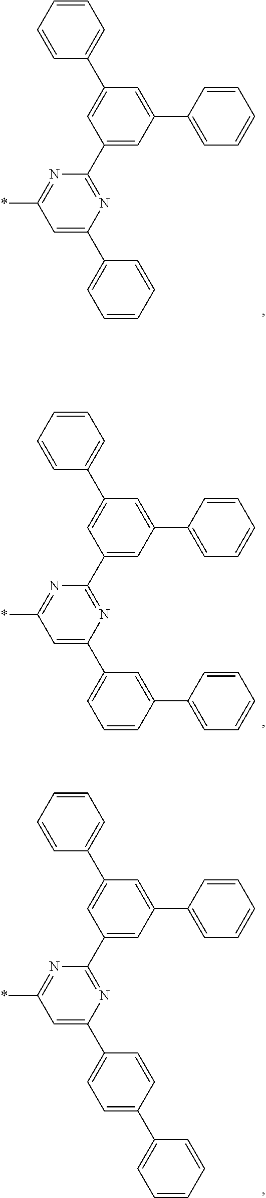 [SMILES: *c1cc(-c2ccccc2)nc(-c2cc(-c3ccccc3)cc(-c3ccccc3)c2)n1 ; *c1cc(-c2cccc(-c3ccccc3)c2)nc(-c2cc(-c3ccccc3)cc(-c3ccccc3)c2)n1 ; *c1cc(-c2ccc(-c3ccccc3)cc2)nc(-c2cc(-c3ccccc3)cc(-c3ccccc3)c2)n1]

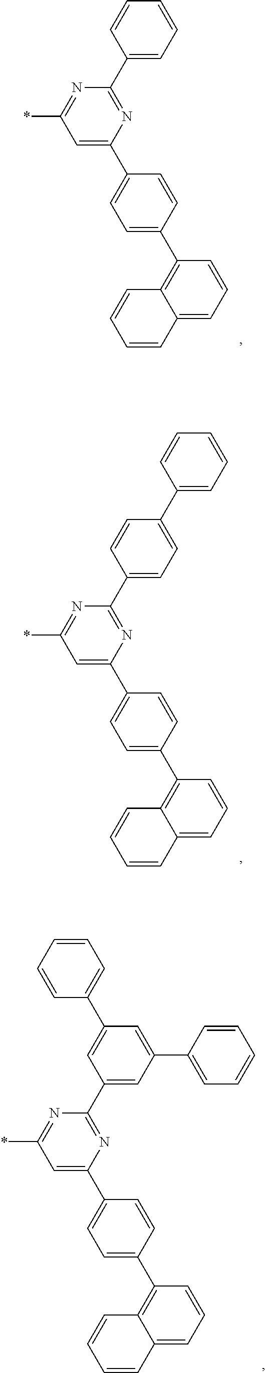 [SMILES: *c1cc(-c2ccc(-c3cccc4ccccc34)cc2)nc(-c2ccccc2)n1 ; *c1cc(-c2ccc(-c3cccc4ccccc34)cc2)nc(-c2ccc(-c3ccccc3)cc2)n1 ; *c1cc(-c2ccc(-c3cccc4ccccc34)cc2)nc(-c2cc(-c3ccccc3)cc(-c3ccccc3)c2)n1]

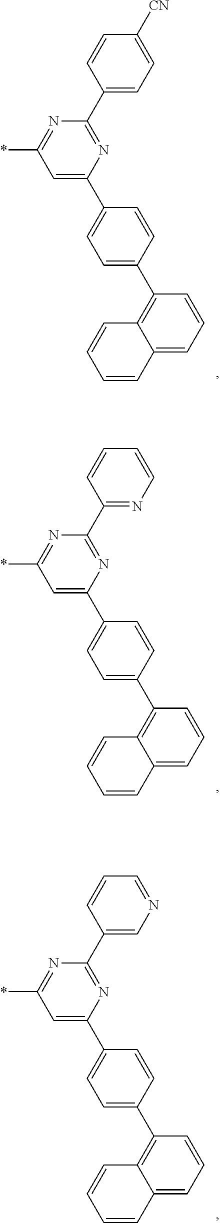 [SMILES: *c1cc(-c2ccc(-c3cccc4ccccc34)cc2)nc(-c2ccc(C#N)cc2)n1 ; *c1cc(-c2ccc(-c3cccc4ccccc34)cc2)nc(-c2ccccn2)n1 ; *c1cc(-c2ccc(-c3cccc4ccccc34)cc2)nc(-c2cccnc2)n1]

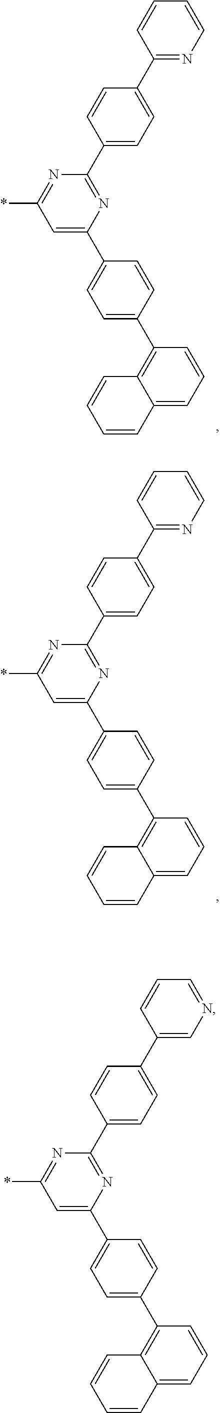

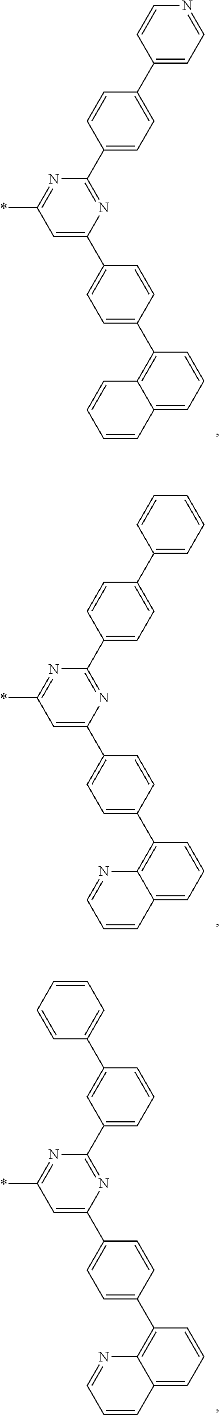 [SMILES: *c1cc(-c2ccc(-c3cccc4ccccc34)cc2)nc(-c2ccc(-c3ccncc3)cc2)n1 ; *c1cc(-c2ccc(-c3cccc4cccnc34)cc2)nc(-c2ccc(-c3ccccc3)cc2)n1 ; *c1cc(-c2ccc(-c3cccc4cccnc34)cc2)nc(-c2cccc(-c3ccccc3)c2)n1]

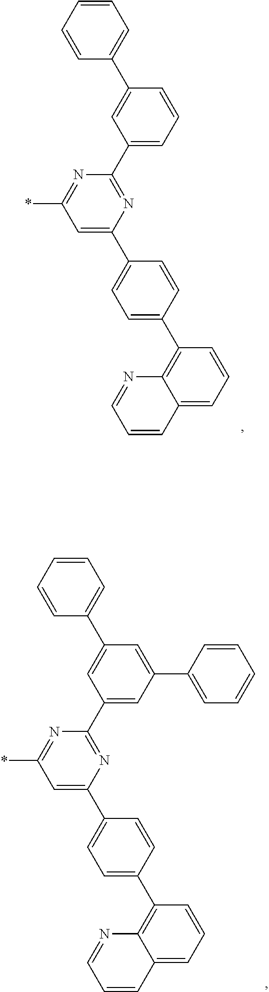 [SMILES: *c1cc(-c2ccc(-c3cccc4cccnc34)cc2)nc(-c2cccc(-c3ccccc3)c2)n1 ; *c1cc(-c2ccc(-c3cccc4cccnc34)cc2)nc(-c2cc(-c3ccccc3)cc(-c3ccccc3)c2)n1]

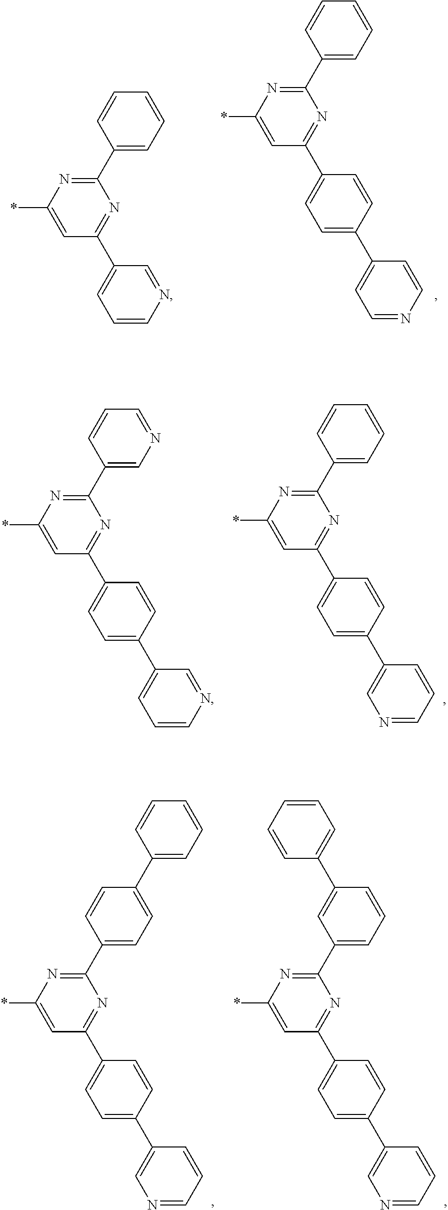 [SMILES: *c1cc(-c2cccnc2)nc(-c2ccccc2)n1 ; *c1cc(-c2ccc(-c3ccncc3)cc2)nc(-c2ccccc2)n1 ; *c1cc(-c2ccc(-c3cccnc3)cc2)nc(-c2cccnc2)n1 ; *c1cc(-c2ccc(-c3cccnc3)cc2)nc(-c2ccccc2)n1 ; *c1cc(-c2ccc(-c3cccnc3)cc2)nc(-c2ccc(-c3ccccc3)cc2)n1 ; *c1cc(-c2ccc(-c3cccnc3)cc2)nc(-c2cccc(-c3ccccc3)c2)n1]

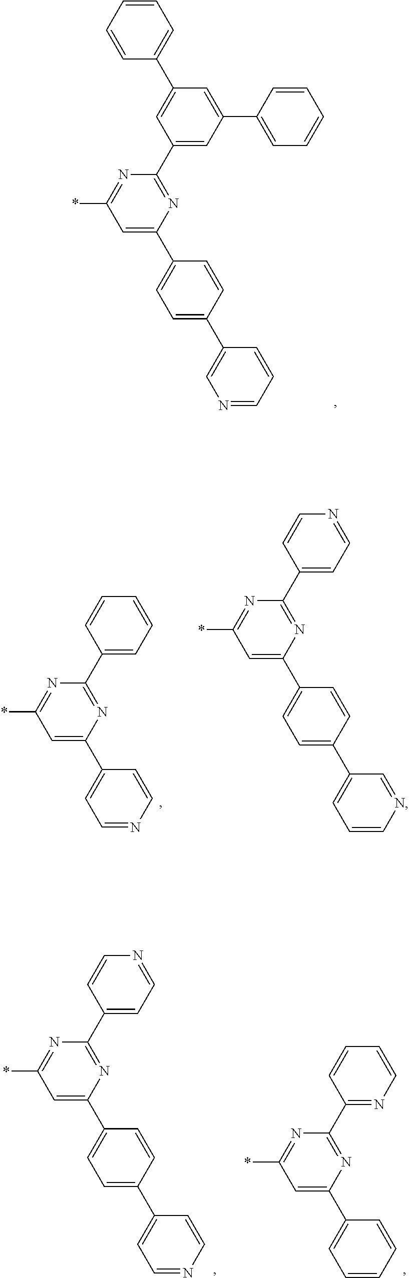 [SMILES: *c1cc(-c2ccc(-c3cccnc3)cc2)nc(-c2cc(-c3ccccc3)cc(-c3ccccc3)c2)n1 ; *c1cc(-c2ccncc2)nc(-c2ccccc2)n1 ; *c1cc(-c2ccc(-c3cccnc3)cc2)nc(-c2ccncc2)n1 ; *c1cc(-c2ccc(-c3ccncc3)cc2)nc(-c2ccncc2)n1 ; *c1cc(-c2ccccc2)nc(-c2ccccn2)n1]

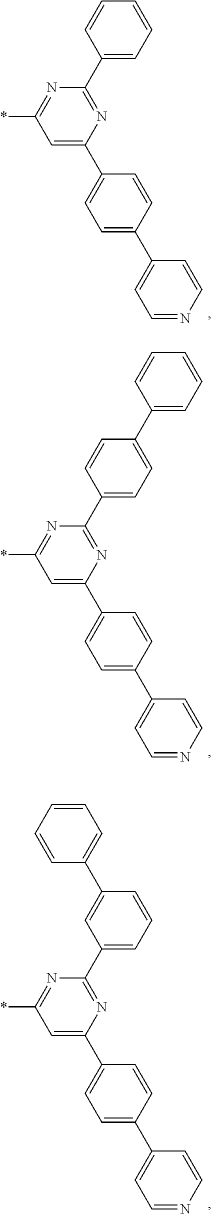

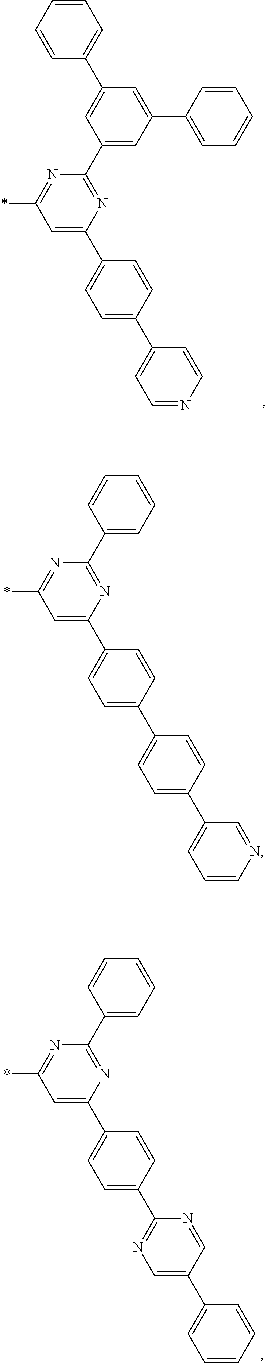 [SMILES: *c1cc(-c2ccc(-c3ccncc3)cc2)nc(-c2cc(-c3ccccc3)cc(-c3ccccc3)c2)n1 ; *c1cc(-c2ccc(-c3ccc(-c4cccnc4)cc3)cc2)nc(-c2ccccc2)n1 ; *c1cc(-c2ccc(-c3ncc(-c4ccccc4)cn3)cc2)nc(-c2ccccc2)n1]

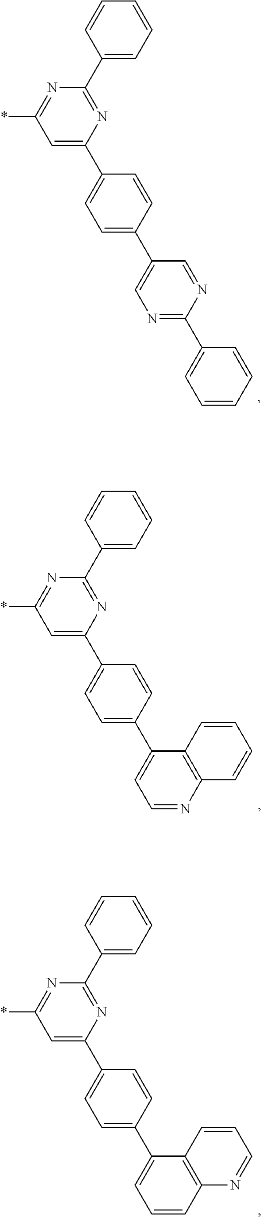 [SMILES: *c1cc(-c2ccc(-c3cnc(-c4ccccc4)nc3)cc2)nc(-c2ccccc2)n1 ; *c1cc(-c2ccc(-c3ccnc4ccccc34)cc2)nc(-c2ccccc2)n1 ; *c1cc(-c2ccc(-c3cccc4ncccc34)cc2)nc(-c2ccccc2)n1]

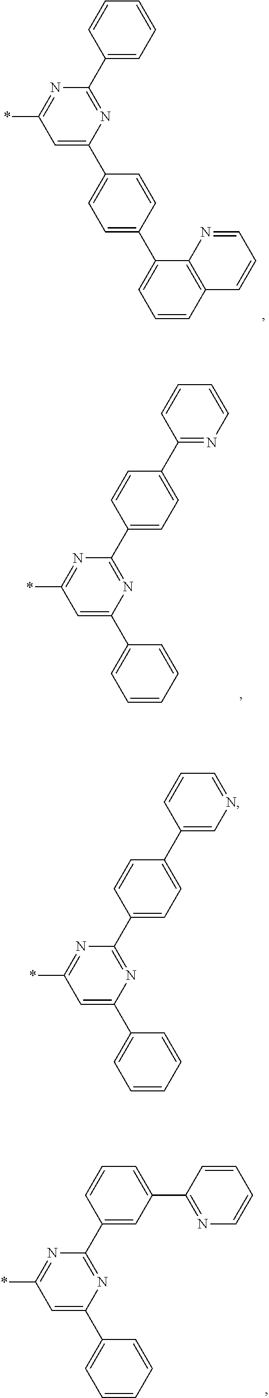 [SMILES: *c1cc(-c2ccc(-c3cccc4cccnc34)cc2)nc(-c2ccccc2)n1 ; *c1cc(-c2ccccc2)nc(-c2ccc(-c3ccccn3)cc2)n1 ; *c1cc(-c2ccccc2)nc(-c2ccc(-c3cccnc3)cc2)n1 ; *c1cc(-c2ccccc2)nc(-c2cccc(-c3ccccn3)c2)n1]

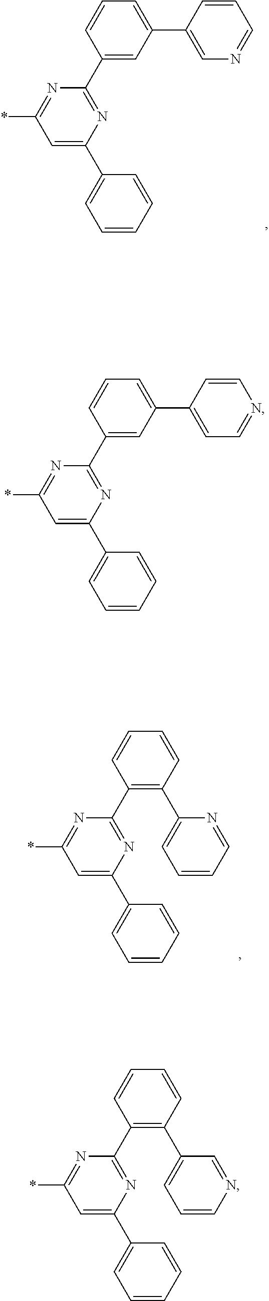 [SMILES: *c1cc(-c2ccccc2)nc(-c2cccc(-c3cccnc3)c2)n1 ; *c1cc(-c2ccccc2)nc(-c2cccc(-c3ccncc3)c2)n1 ; *c1cc(-c2ccccc2)nc(-c2ccccc2-c2ccccn2)n1 ; *c1cc(-c2ccccc2)nc(-c2ccccc2-c2cccnc2)n1]

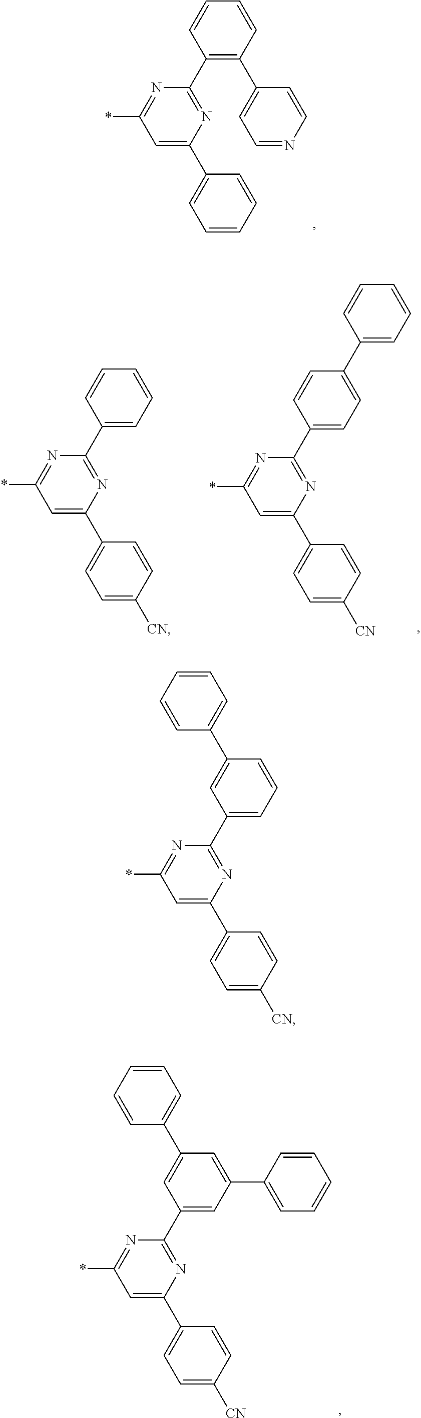 [SMILES: *c1cc(-c2ccccc2)nc(-c2ccccc2-c2ccncc2)n1 ; *c1cc(-c2ccc(C#N)cc2)nc(-c2ccccc2)n1 ; *c1cc(-c2ccc(C#N)cc2)nc(-c2ccc(-c3ccccc3)cc2)n1 ; *c1cc(-c2ccc(C#N)cc2)nc(-c2cccc(-c3ccccc3)c2)n1 ; *c1cc(-c2ccc(C#N)cc2)nc(-c2cc(-c3ccccc3)cc(-c3ccccc3)c2)n1]

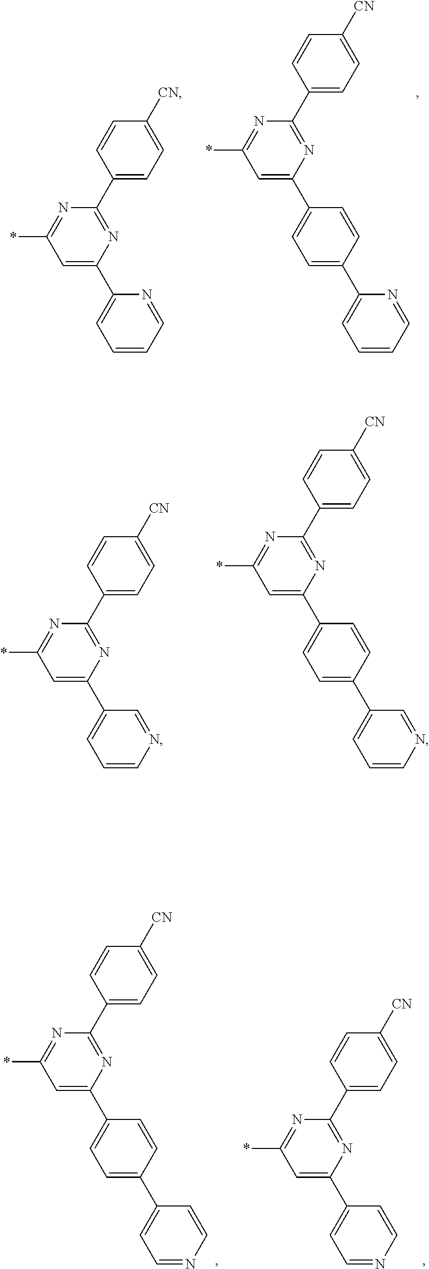

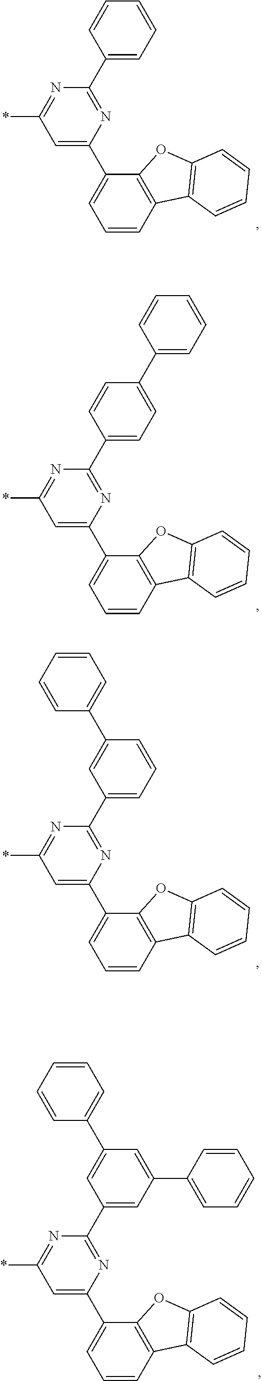

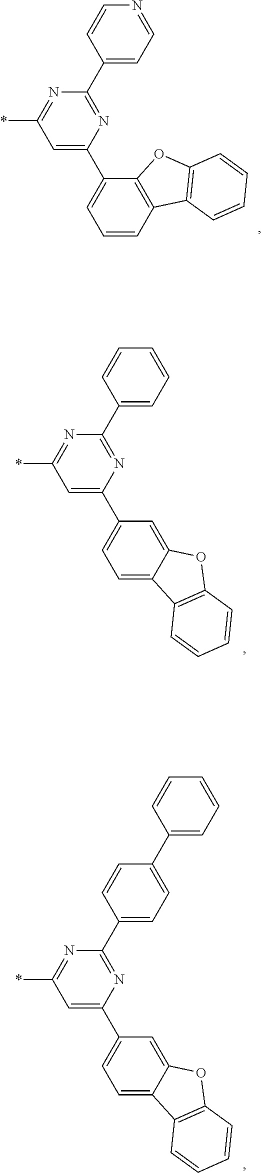 [SMILES: *c1cc(-c2cccc3c2oc2ccccc23)nc(-c2ccncc2)n1 ; *c1cc(-c2ccc3c(c2)oc2ccccc23)nc(-c2ccccc2)n1 ; *c1cc(-c2ccc3c(c2)oc2ccccc23)nc(-c2ccc(-c3ccccc3)cc2)n1]

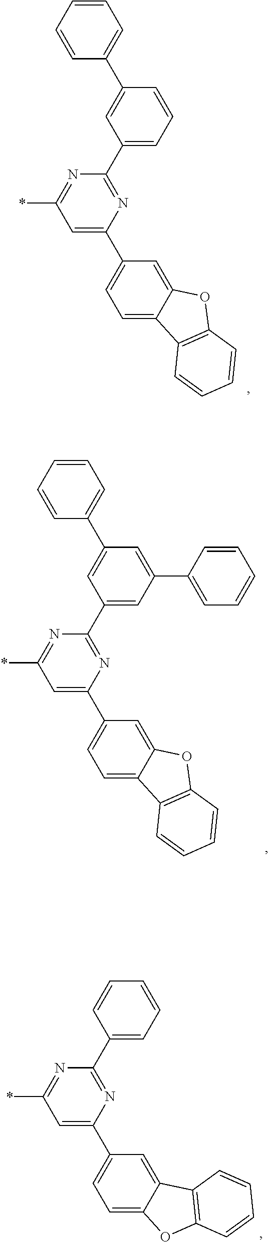 [SMILES: *c1cc(-c2ccc3c(c2)oc2ccccc23)nc(-c2cccc(-c3ccccc3)c2)n1 ; *c1cc(-c2ccc3c(c2)oc2ccccc23)nc(-c2cc(-c3ccccc3)cc(-c3ccccc3)c2)n1 ; *c1cc(-c2ccc3oc4ccccc4c3c2)nc(-c2ccccc2)n1]

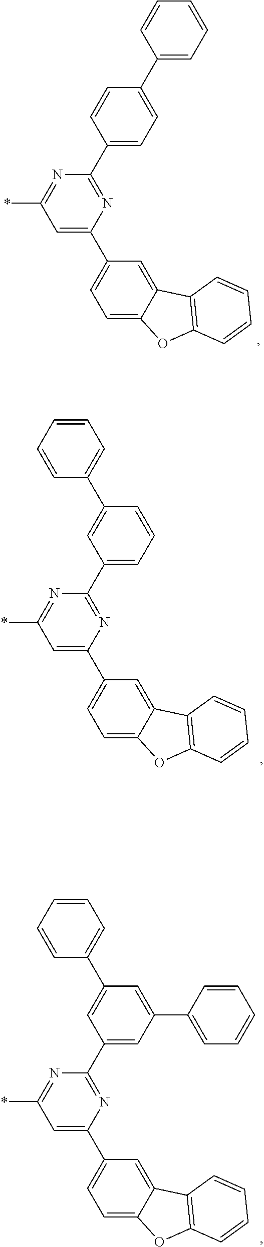 [SMILES: *c1cc(-c2ccc3oc4ccccc4c3c2)nc(-c2ccc(-c3ccccc3)cc2)n1 ; *c1cc(-c2ccc3oc4ccccc4c3c2)nc(-c2cccc(-c3ccccc3)c2)n1 ; *c1cc(-c2ccc3oc4ccccc4c3c2)nc(-c2cc(-c3ccccc3)cc(-c3ccccc3)c2)n1]

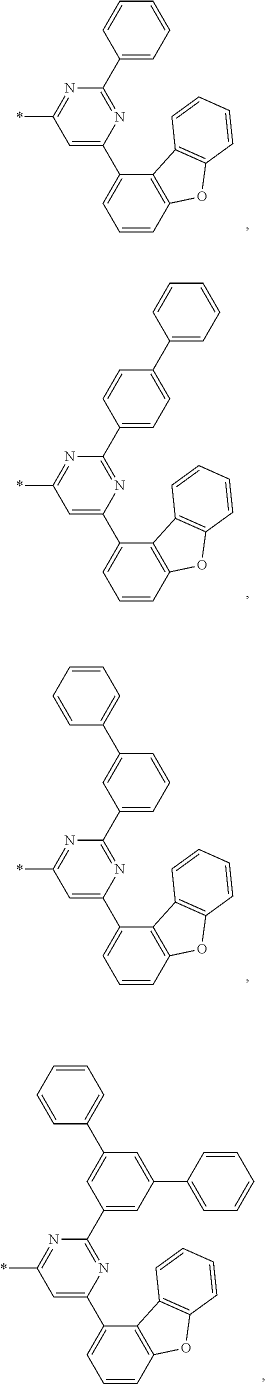 [SMILES: *c1cc(-c2cccc3oc4ccccc4c23)nc(-c2ccccc2)n1 ; *c1cc(-c2cccc3oc4ccccc4c23)nc(-c2ccc(-c3ccccc3)cc2)n1 ; *c1cc(-c2cccc3oc4ccccc4c23)nc(-c2cccc(-c3ccccc3)c2)n1 ; *c1cc(-c2cccc3oc4ccccc4c23)nc(-c2cc(-c3ccccc3)cc(-c3ccccc3)c2)n1]

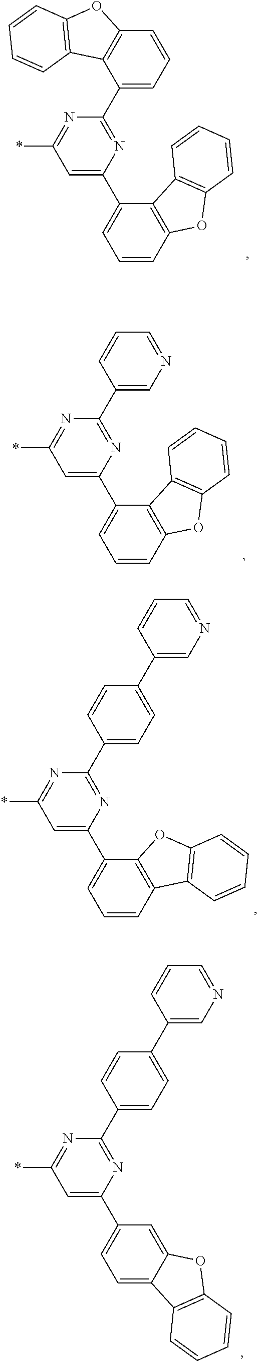

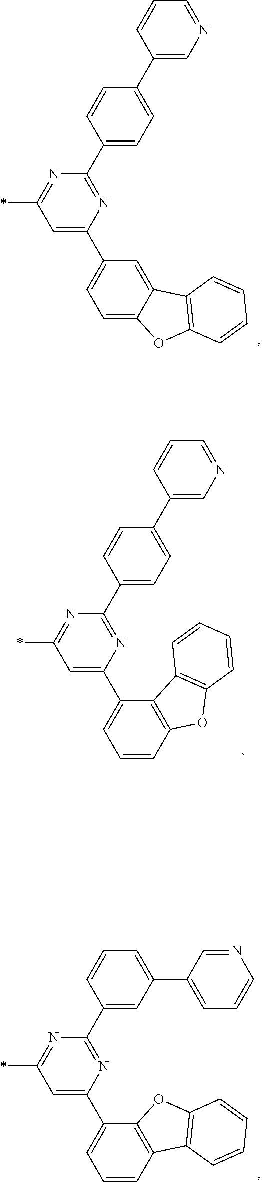 [SMILES: *c1cc(-c2ccc3oc4ccccc4c3c2)nc(-c2ccc(-c3cccnc3)cc2)n1 ; *c1cc(-c2cccc3oc4ccccc4c23)nc(-c2ccc(-c3cccnc3)cc2)n1 ; *c1cc(-c2cccc3c2oc2ccccc23)nc(-c2cccc(-c3cccnc3)c2)n1]

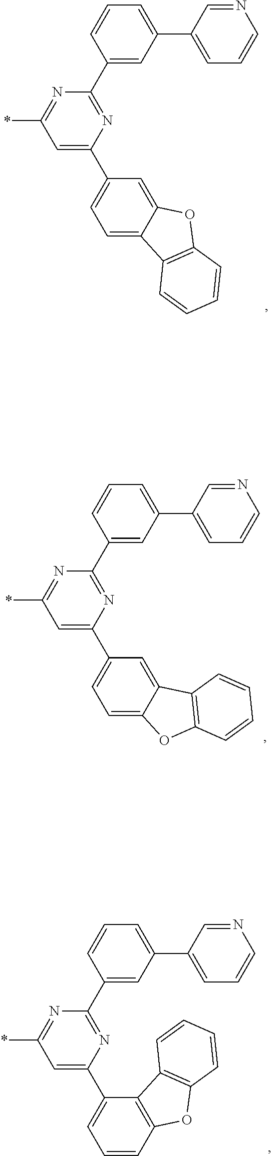 [SMILES: *c1cc(-c2ccc3c(c2)oc2ccccc23)nc(-c2cccc(-c3cccnc3)c2)n1 ; *c1cc(-c2ccc3oc4ccccc4c3c2)nc(-c2cccc(-c3cccnc3)c2)n1 ; *c1cc(-c2cccc3oc4ccccc4c23)nc(-c2cccc(-c3cccnc3)c2)n1]

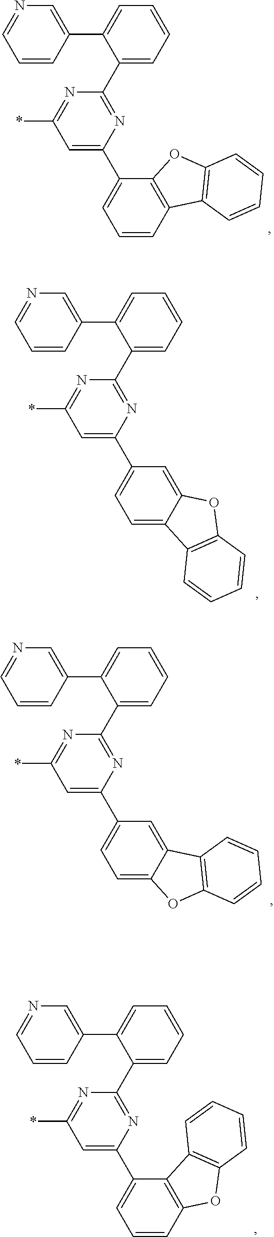 [SMILES: *c1cc(-c2cccc3c2oc2ccccc23)nc(-c2ccccc2-c2cccnc2)n1 ; *c1cc(-c2ccc3c(c2)oc2ccccc23)nc(-c2ccccc2-c2cccnc2)n1 ; *c1cc(-c2ccc3oc4ccccc4c3c2)nc(-c2ccccc2-c2cccnc2)n1 ; *c1cc(-c2cccc3oc4ccccc4c23)nc(-c2ccccc2-c2cccnc2)n1]

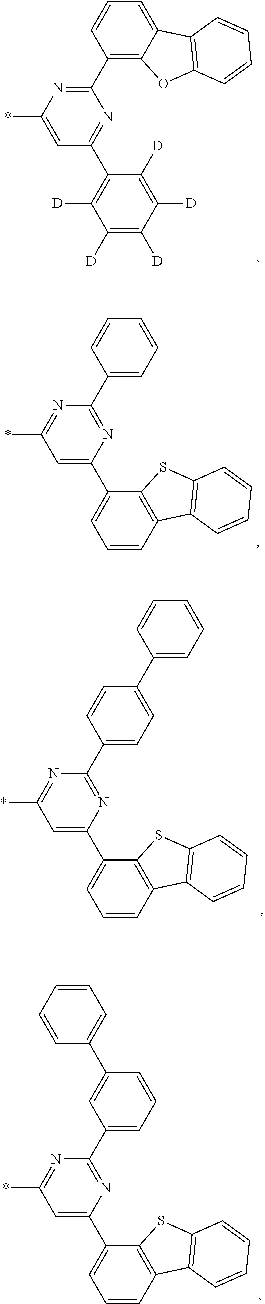 [SMILES: *c1cc(-c2c([2H])c([2H])c([2H])c([2H])c2[2H])nc(-c2cccc3c2oc2ccccc23)n1 ; *c1cc(-c2cccc3c2sc2ccccc23)nc(-c2ccccc2)n1 ; *c1cc(-c2cccc3c2sc2ccccc23)nc(-c2ccc(-c3ccccc3)cc2)n1 ; *c1cc(-c2cccc3c2sc2ccccc23)nc(-c2cccc(-c3ccccc3)c2)n1]

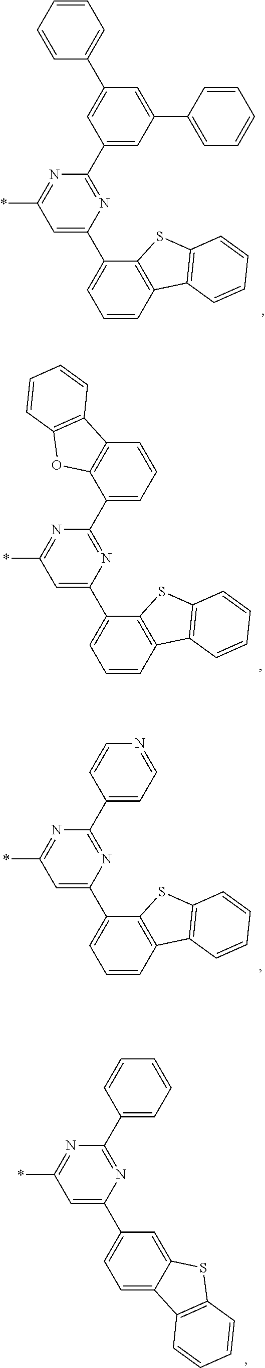 [SMILES: *c1cc(-c2cccc3c2sc2ccccc23)nc(-c2cc(-c3ccccc3)cc(-c3ccccc3)c2)n1 ; *c1cc(-c2cccc3c2sc2ccccc23)nc(-c2cccc3c2oc2ccccc23)n1 ; *c1cc(-c2cccc3c2sc2ccccc23)nc(-c2ccncc2)n1 ; *c1cc(-c2ccc3c(c2)sc2ccccc23)nc(-c2ccccc2)n1]

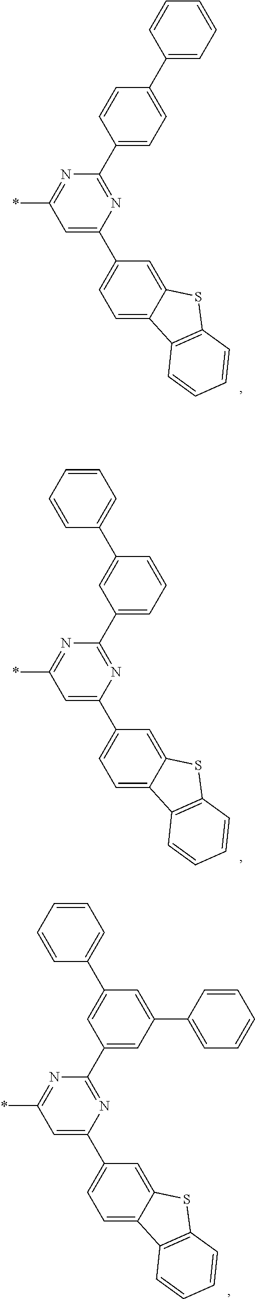 [SMILES: *c1cc(-c2ccc3c(c2)sc2ccccc23)nc(-c2ccc(-c3ccccc3)cc2)n1 ; *c1cc(-c2ccc3c(c2)sc2ccccc23)nc(-c2cccc(-c3ccccc3)c2)n1 ; *c1cc(-c2ccc3c(c2)sc2ccccc23)nc(-c2cc(-c3ccccc3)cc(-c3ccccc3)c2)n1]

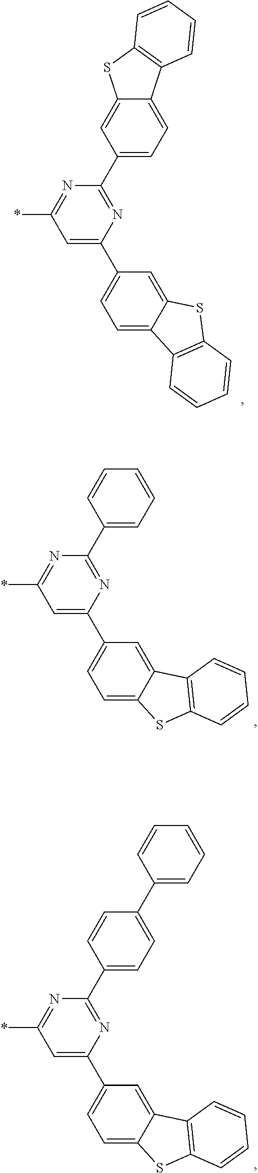 [SMILES: *c1cc(-c2ccc3c(c2)sc2ccccc23)nc(-c2ccc3c(c2)sc2ccccc23)n1 ; *c1cc(-c2ccc3sc4ccccc4c3c2)nc(-c2ccccc2)n1 ; *c1cc(-c2ccc3sc4ccccc4c3c2)nc(-c2ccc(-c3ccccc3)cc2)n1]

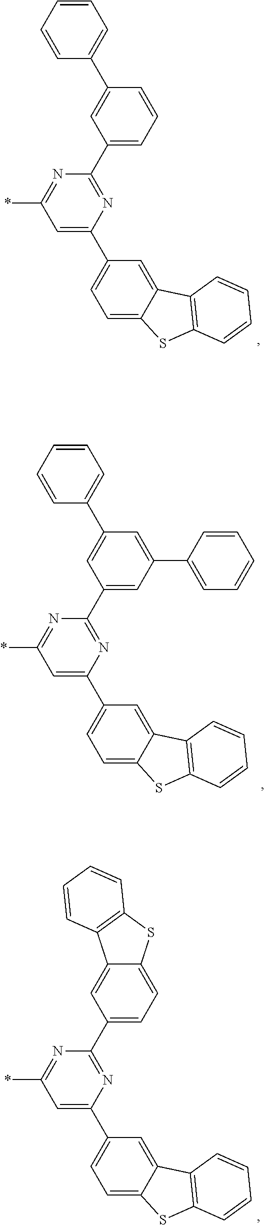 [SMILES: *c1cc(-c2ccc3sc4ccccc4c3c2)nc(-c2cccc(-c3ccccc3)c2)n1 ; *c1cc(-c2ccc3sc4ccccc4c3c2)nc(-c2cc(-c3ccccc3)cc(-c3ccccc3)c2)n1 ; *c1cc(-c2ccc3sc4ccccc4c3c2)nc(-c2ccc3sc4ccccc4c3c2)n1]

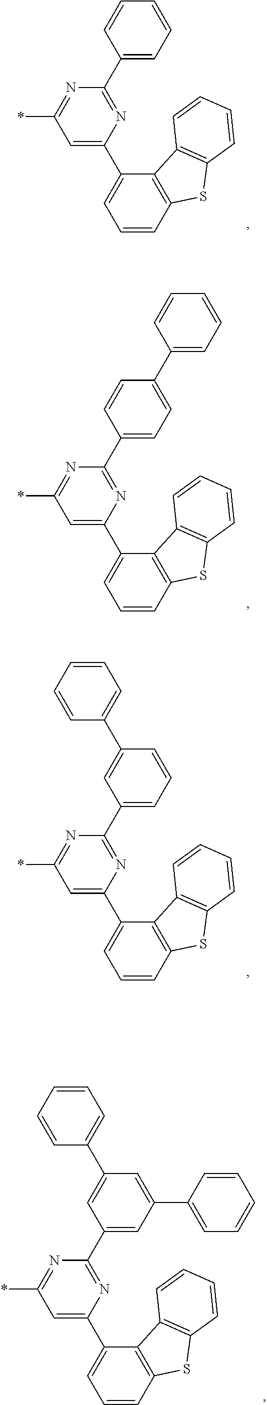 [SMILES: *c1cc(-c2cccc3sc4ccccc4c23)nc(-c2ccccc2)n1 ; *c1cc(-c2cccc3sc4ccccc4c23)nc(-c2ccc(-c3ccccc3)cc2)n1 ; *c1cc(-c2cccc3sc4ccccc4c23)nc(-c2cccc(-c3ccccc3)c2)n1 ; *c1cc(-c2cccc3sc4ccccc4c23)nc(-c2cc(-c3ccccc3)cc(-c3ccccc3)c2)n1]

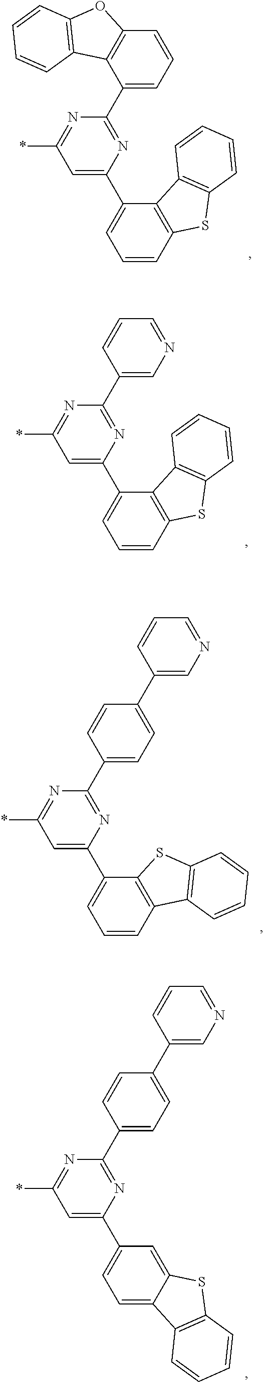 [SMILES: *c1cc(-c2cccc3sc4ccccc4c23)nc(-c2cccc3oc4ccccc4c23)n1 ; *c1cc(-c2cccc3sc4ccccc4c23)nc(-c2cccnc2)n1 ; *c1cc(-c2cccc3c2sc2ccccc23)nc(-c2ccc(-c3cccnc3)cc2)n1 ; *c1cc(-c2ccc3c(c2)sc2ccccc23)nc(-c2ccc(-c3cccnc3)cc2)n1]

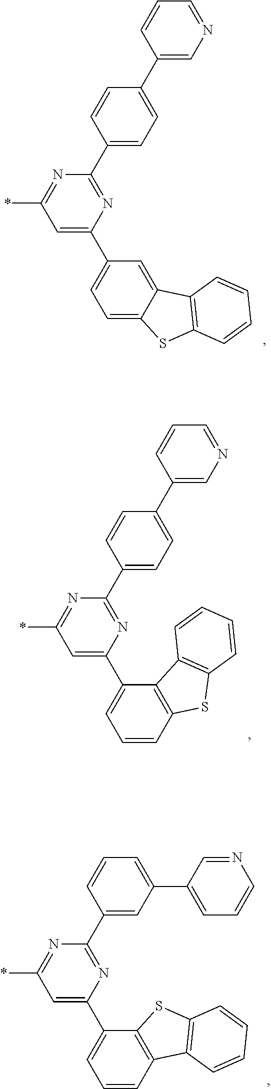 [SMILES: *c1cc(-c2ccc3sc4ccccc4c3c2)nc(-c2ccc(-c3cccnc3)cc2)n1 ; *c1cc(-c2cccc3sc4ccccc4c23)nc(-c2ccc(-c3cccnc3)cc2)n1 ; *c1cc(-c2cccc3c2sc2ccccc23)nc(-c2cccc(-c3cccnc3)c2)n1]

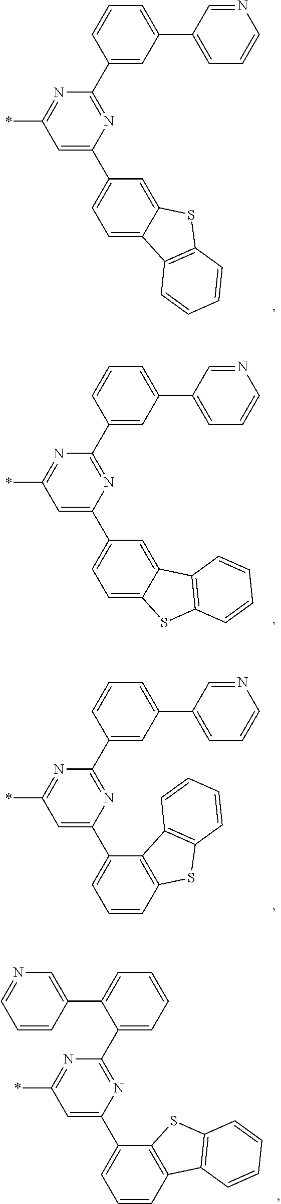 [SMILES: *c1cc(-c2ccc3c(c2)sc2ccccc23)nc(-c2cccc(-c3cccnc3)c2)n1 ; *c1cc(-c2ccc3sc4ccccc4c3c2)nc(-c2cccc(-c3cccnc3)c2)n1 ; *c1cc(-c2cccc3sc4ccccc4c23)nc(-c2cccc(-c3cccnc3)c2)n1 ; *c1cc(-c2cccc3c2sc2ccccc23)nc(-c2ccccc2-c2cccnc2)n1]

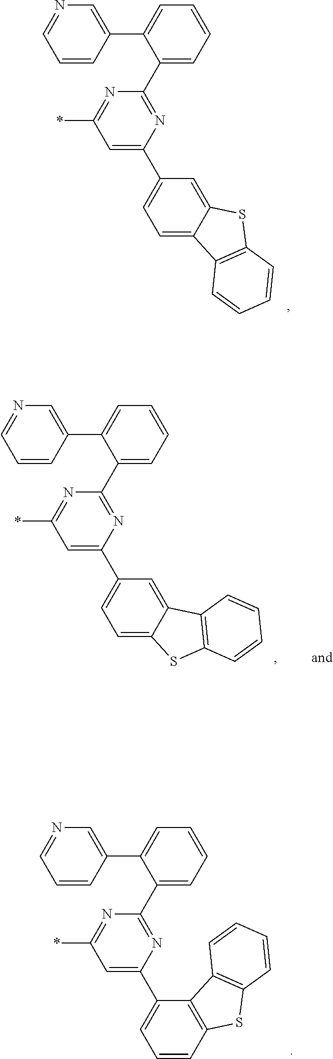 [SMILES: *c1cc(-c2ccc3c(c2)sc2ccccc23)nc(-c2ccccc2-c2cccnc2)n1 ; *c1cc(-c2ccc3sc4ccccc4c3c2)nc(-c2ccccc2-c2cccnc2)n1 ; *c1cc(-c2cccc3sc4ccccc4c23)nc(-c2ccccc2-c2cccnc2)n1]

Preferably, G² of Formula (I) is selected from the group consisting of:

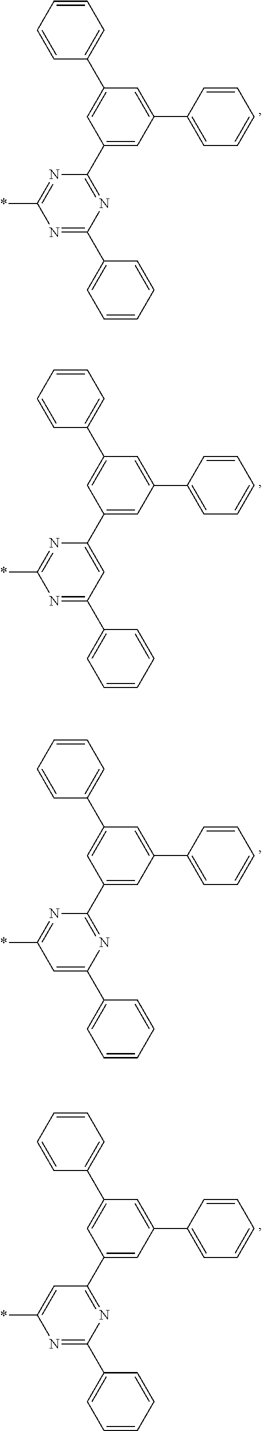 [SMILES: *c1nc(-c2ccccc2)nc(-c2cc(-c3ccccc3)cc(-c3ccccc3)c2)n1 ; *c1nc(-c2ccccc2)cc(-c2cc(-c3ccccc3)cc(-c3ccccc3)c2)n1 ; *c1cc(-c2ccccc2)nc(-c2cc(-c3ccccc3)cc(-c3ccccc3)c2)n1 ; *c1cc(-c2cc(-c3ccccc3)cc(-c3ccccc3)c2)nc(-c2ccccc2)n1]

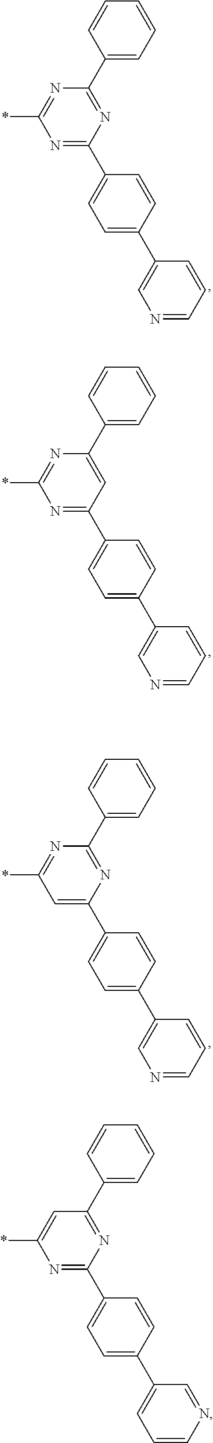 [SMILES: *c1nc(-c2ccccc2)nc(-c2ccc(-c3cccnc3)cc2)n1 ; *c1nc(-c2ccccc2)cc(-c2ccc(-c3cccnc3)cc2)n1 ; *c1cc(-c2ccc(-c3cccnc3)cc2)nc(-c2ccccc2)n1 ; *c1cc(-c2ccccc2)nc(-c2ccc(-c3cccnc3)cc2)n1]

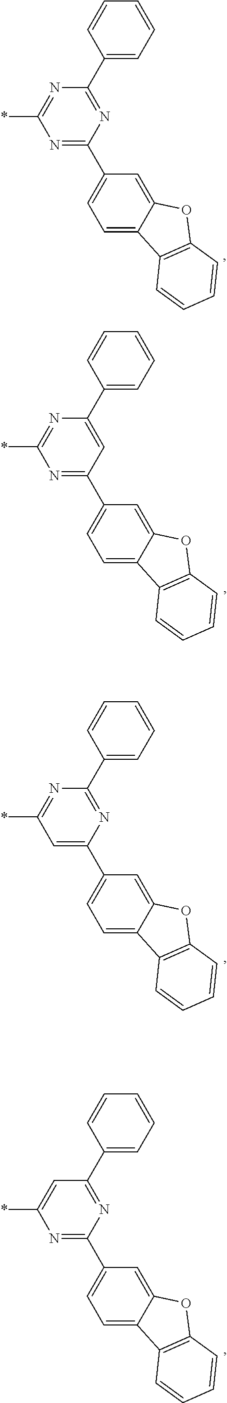

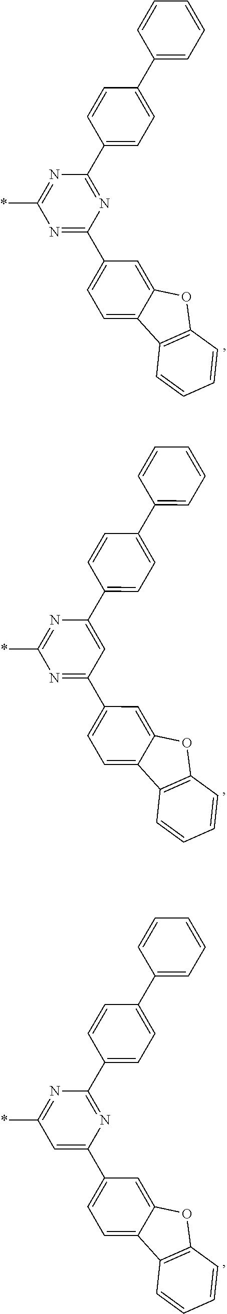 [SMILES: *c1nc(-c2ccc(-c3ccccc3)cc2)nc(-c2ccc3c(c2)oc2ccccc23)n1 ; *c1nc(-c2ccc(-c3ccccc3)cc2)cc(-c2ccc3c(c2)oc2ccccc23)n1 ; *c1cc(-c2ccc3c(c2)oc2ccccc23)nc(-c2ccc(-c3ccccc3)cc2)n1]

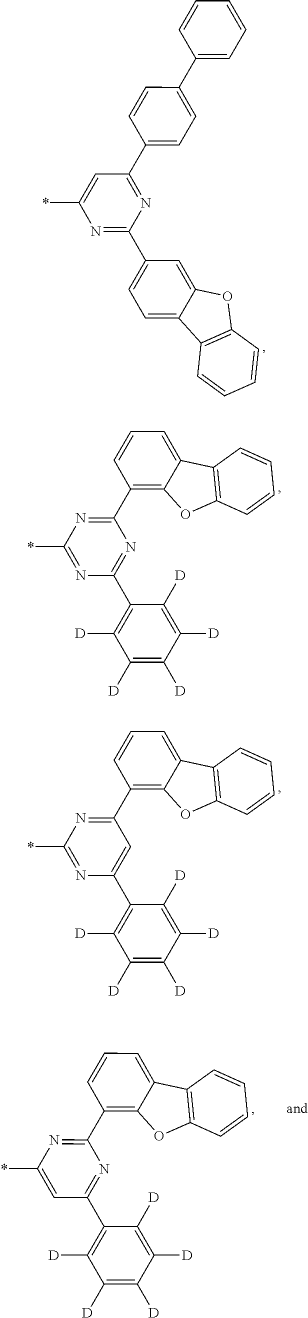

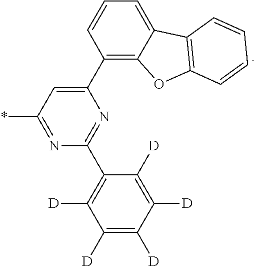 [SMILES: *c1cc(-c2cccc3c2oc2ccccc23)nc(-c2c([2H])c([2H])c([2H])c([2H])c2[2H])n1]

Preferably, the arylene groups having 6 to 60 ring carbon atoms represented by said L¹ to L⁴ are each independently selected from the group consisting of:

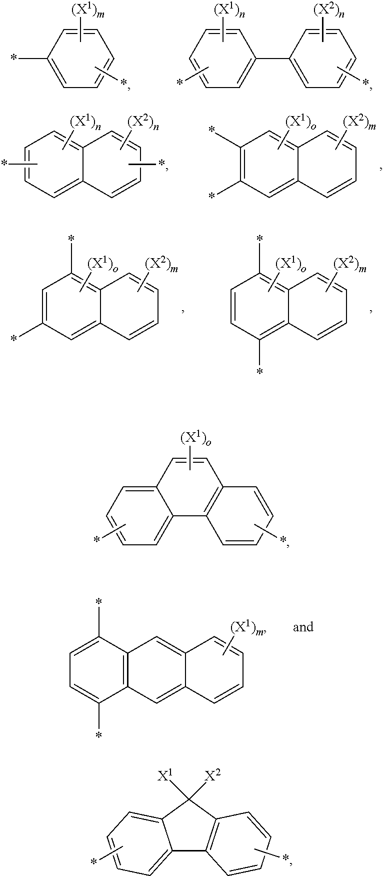

wherein m is an integer from 1 to 4, n is an integer from 1 to 3, and o is an integer 1 or 2.

X¹ to X² are each independently selected from the group consisting of: a hydrogen atom, a deuterium atom, a halo group, a cyano group, a nitro group, an alkyl group having 1 to 12 carbon atoms, an alkenyl group having 2 to 12 carbon atoms, an alkynyl group having 2 to 12 carbon atoms, an alkoxy group having 1 to 12 carbon atoms, an aryl group having 6 to 30 ring carbon atoms, a heteroaryl group having 3 to 30 ring carbon atoms, and an aryloxy group having 6 to 30 ring carbon atoms.

More preferably, said Y¹ to Y³ are each independently selected from the group consisting of: a hydrogen atom, a deuterium atom, a methyl group, an ethyl group, a propyl group, a butyl group, a pentyl group, a hexyl group, a phenyl group, a biphenyl group, and a napthyl group.

In this specification, said “arylene group having 6 to 60 ring carbon atoms” denoted by L¹, L², L³, or L⁴ may be an unsubstituted arylene group having 6 to 60 ring carbon atoms or an arylene group having 6 to 60 ring carbon atoms substituted with a substituent. The substituent on the arylene group may be any one of X¹ to X² as stated above.

In this specification, said “alkyl group” may be an unsubstituted alkyl group or an alkyl group substituted with a substituent, said “alkenyl group” may be an unsubstituted alkenyl group or an alkenyl group substituted with a substituent, and said “alkynyl group” may be an unsubstituted alkynyl group or an alkynyl group substituted with a substituent. The substituent on the alkyl group, alkenyl group, or alkynyl group may be, for example, but not limited to a deuterium atom.

For example, the compound may be selected from the group consisting of:

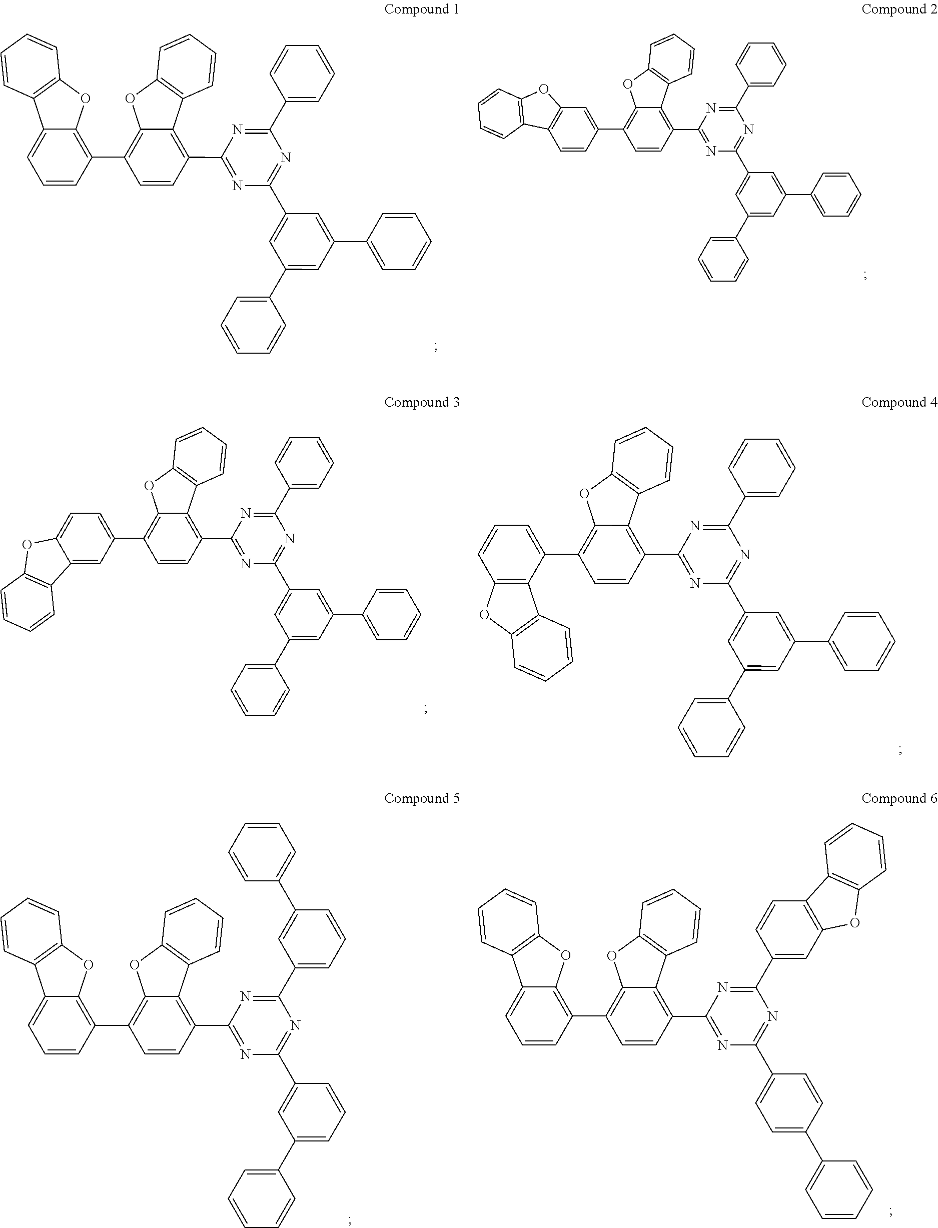

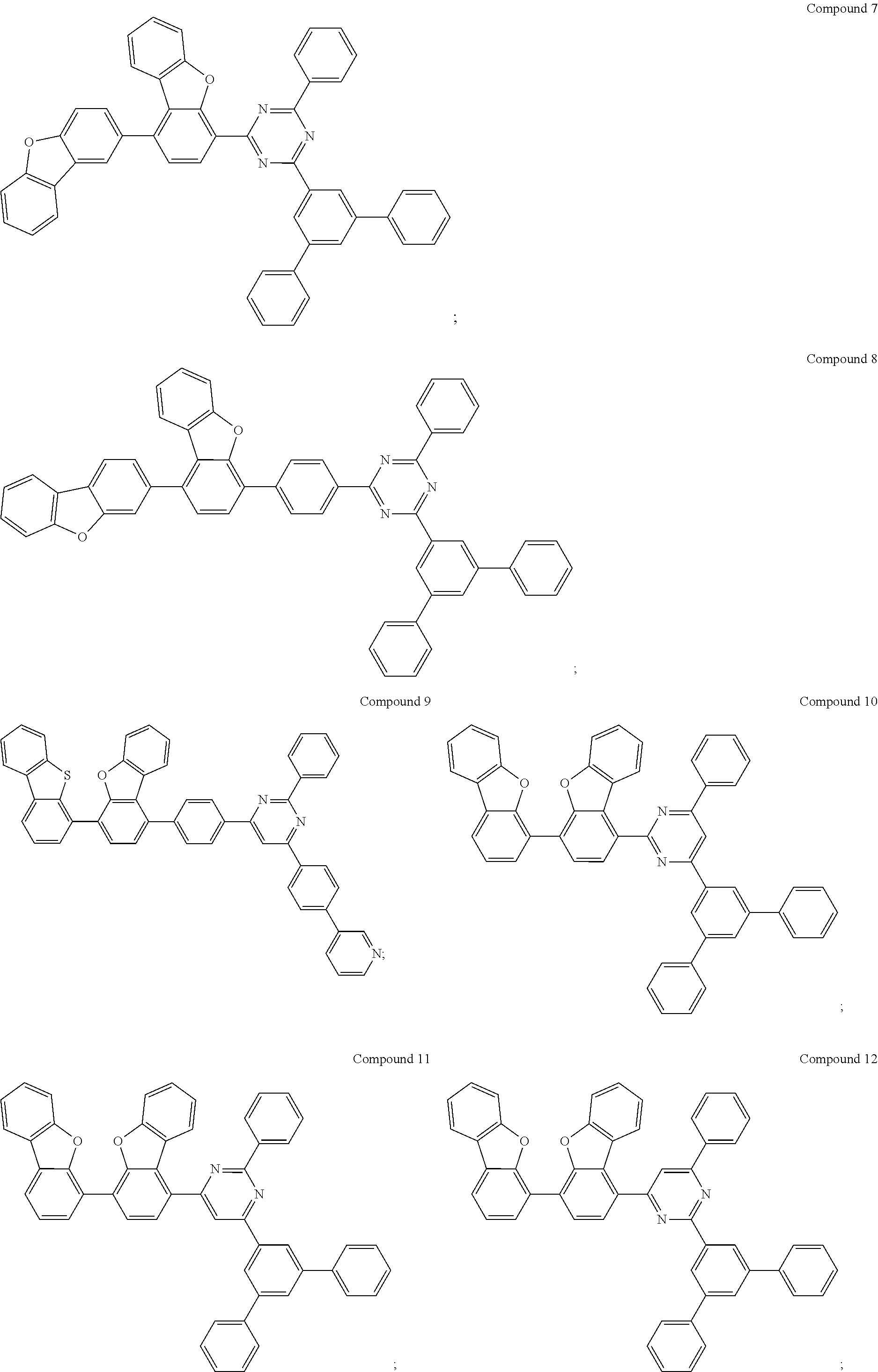

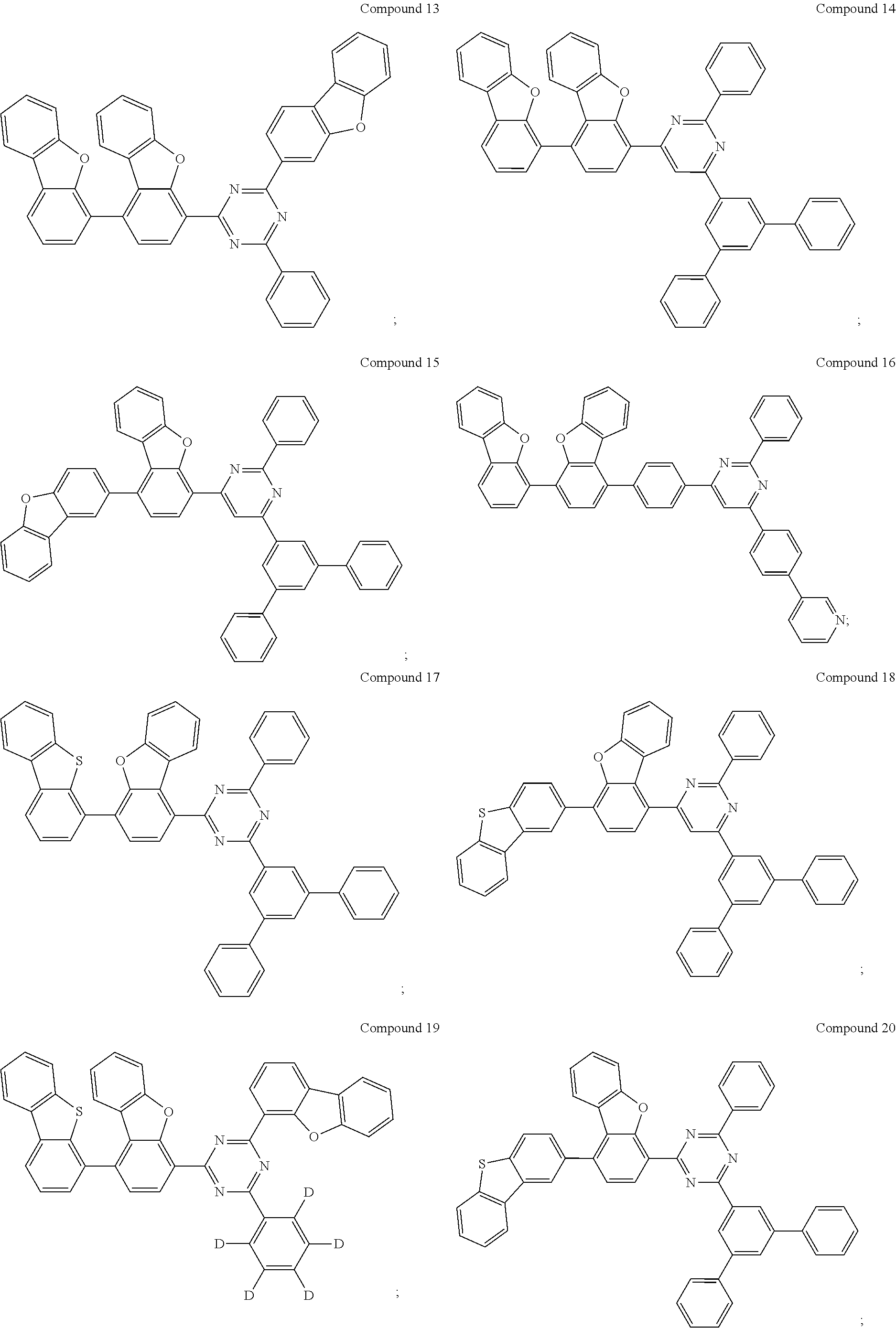

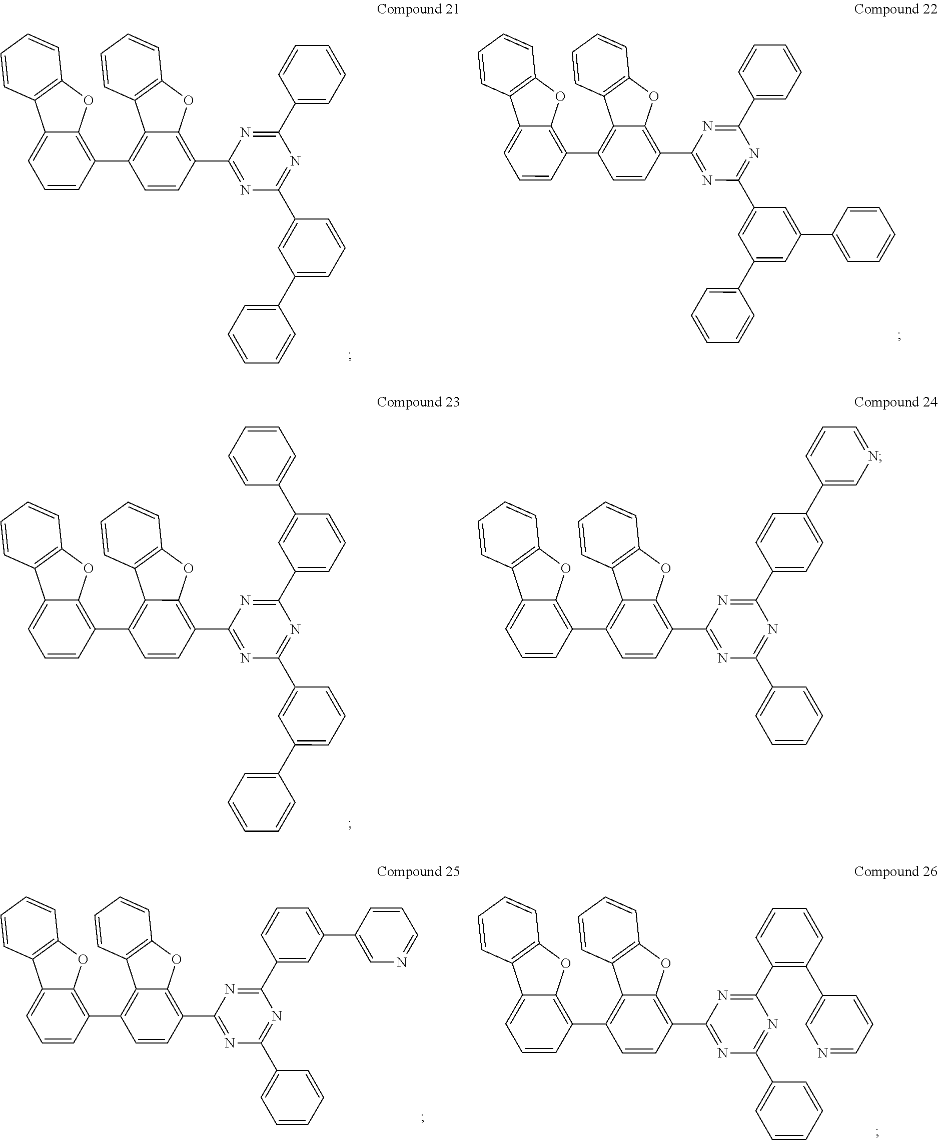

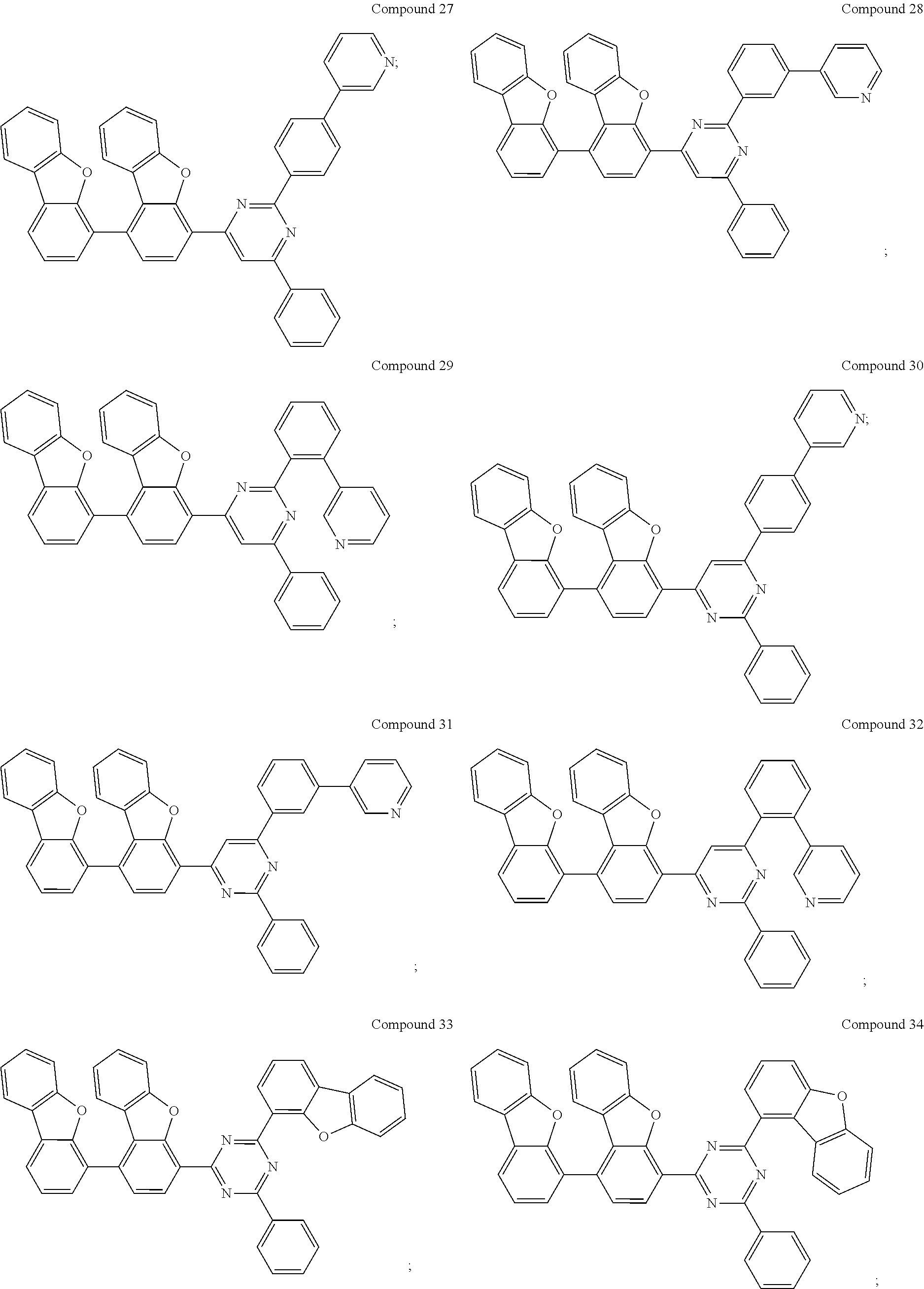

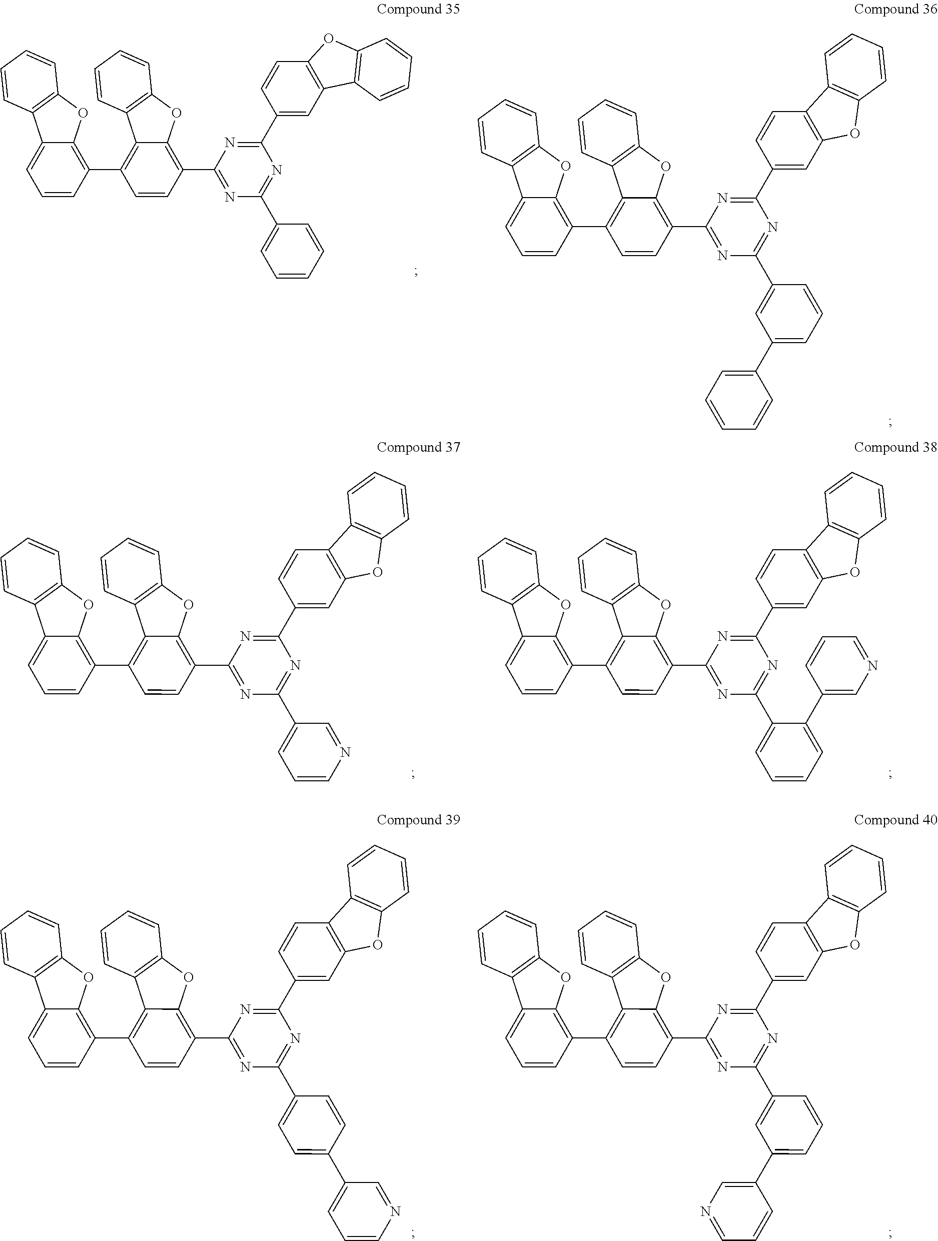

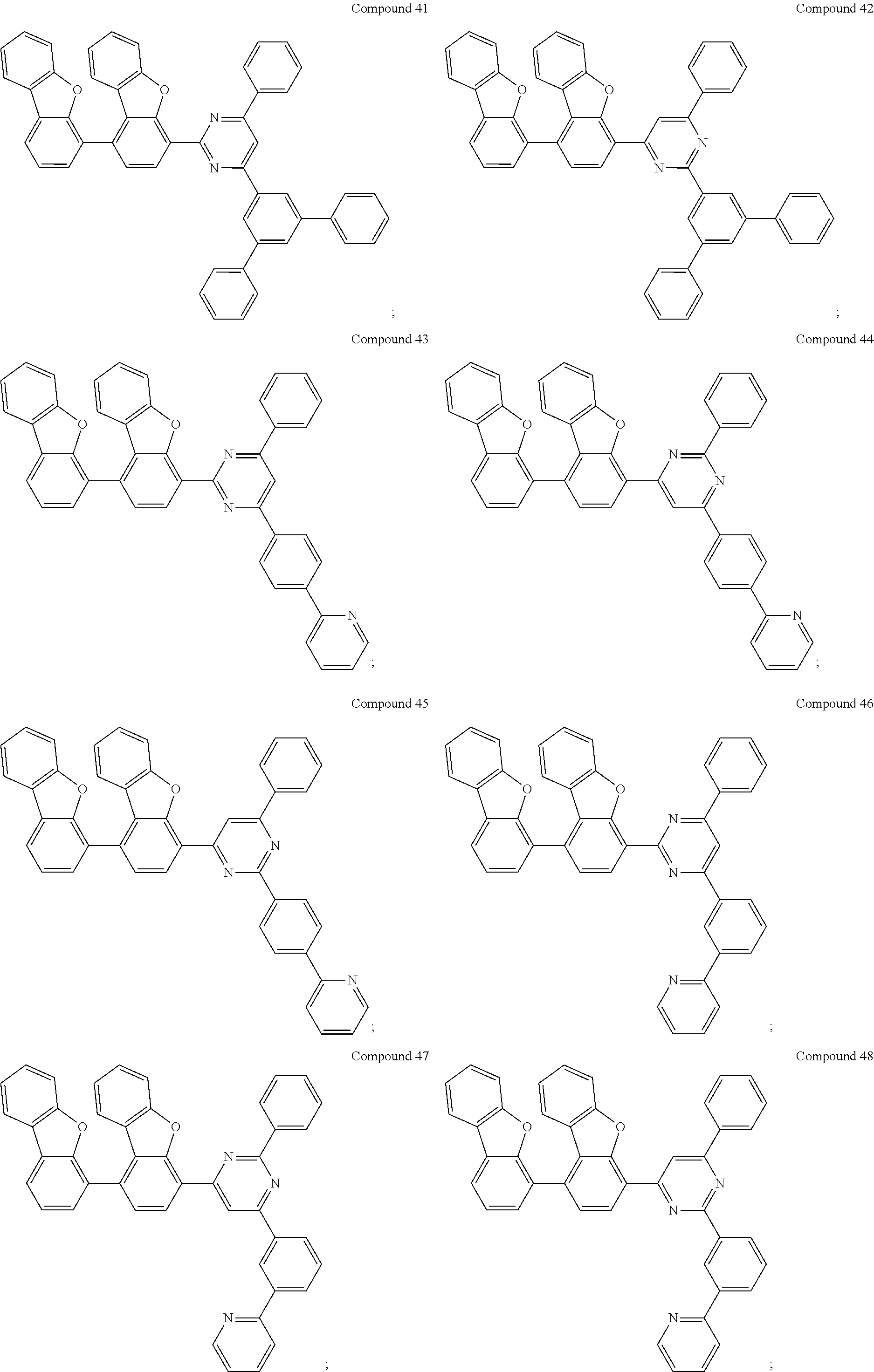

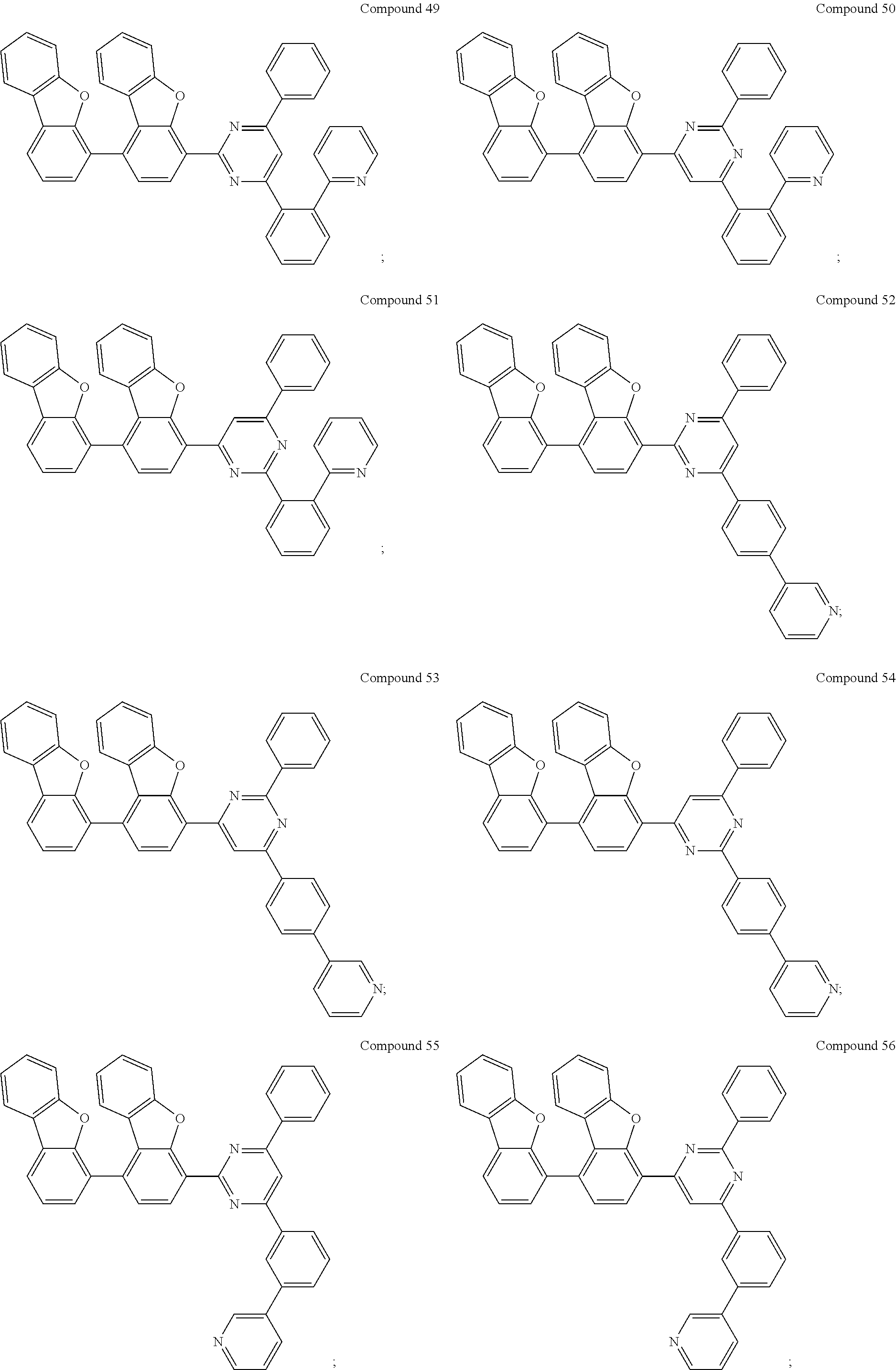

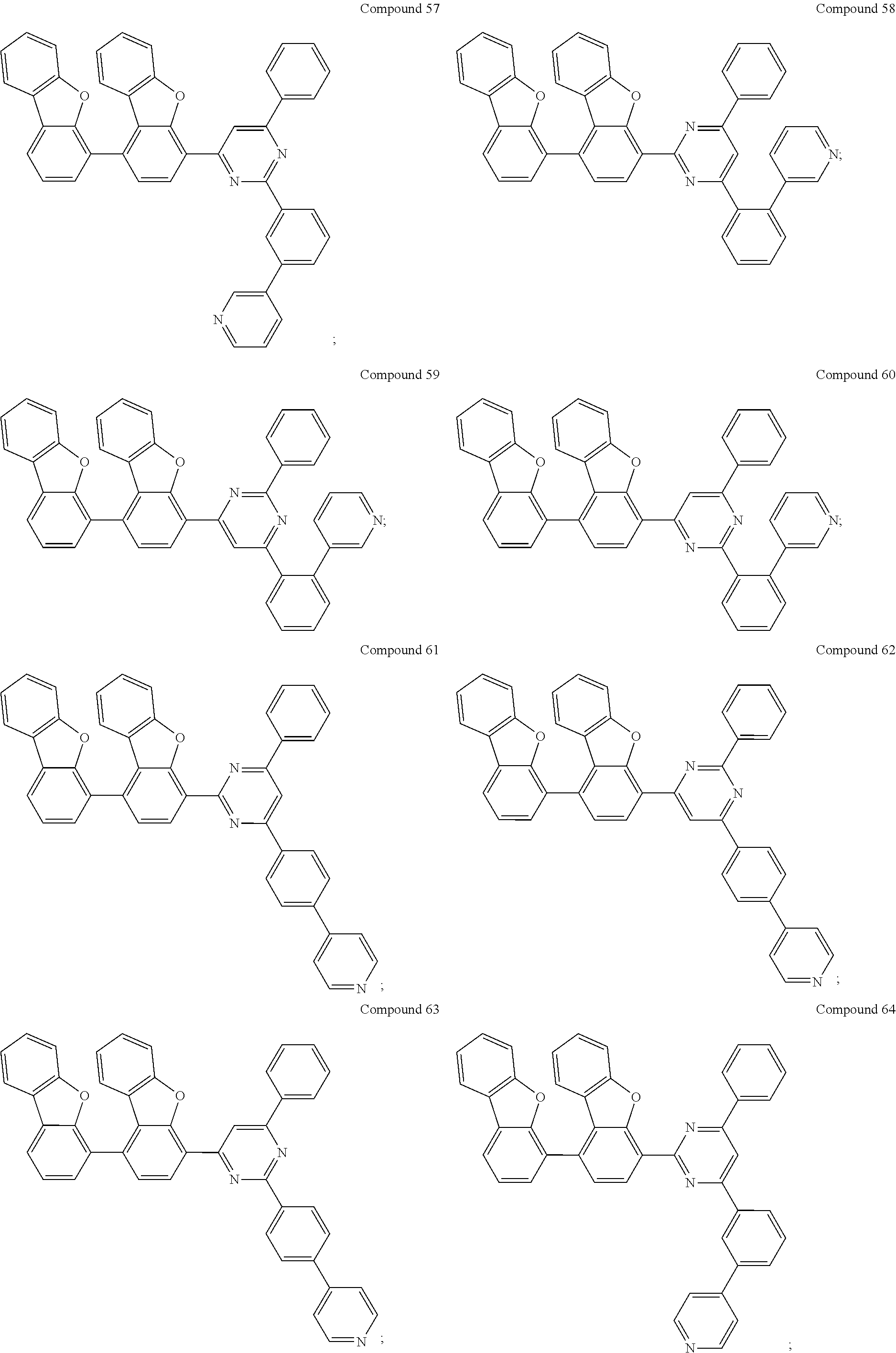

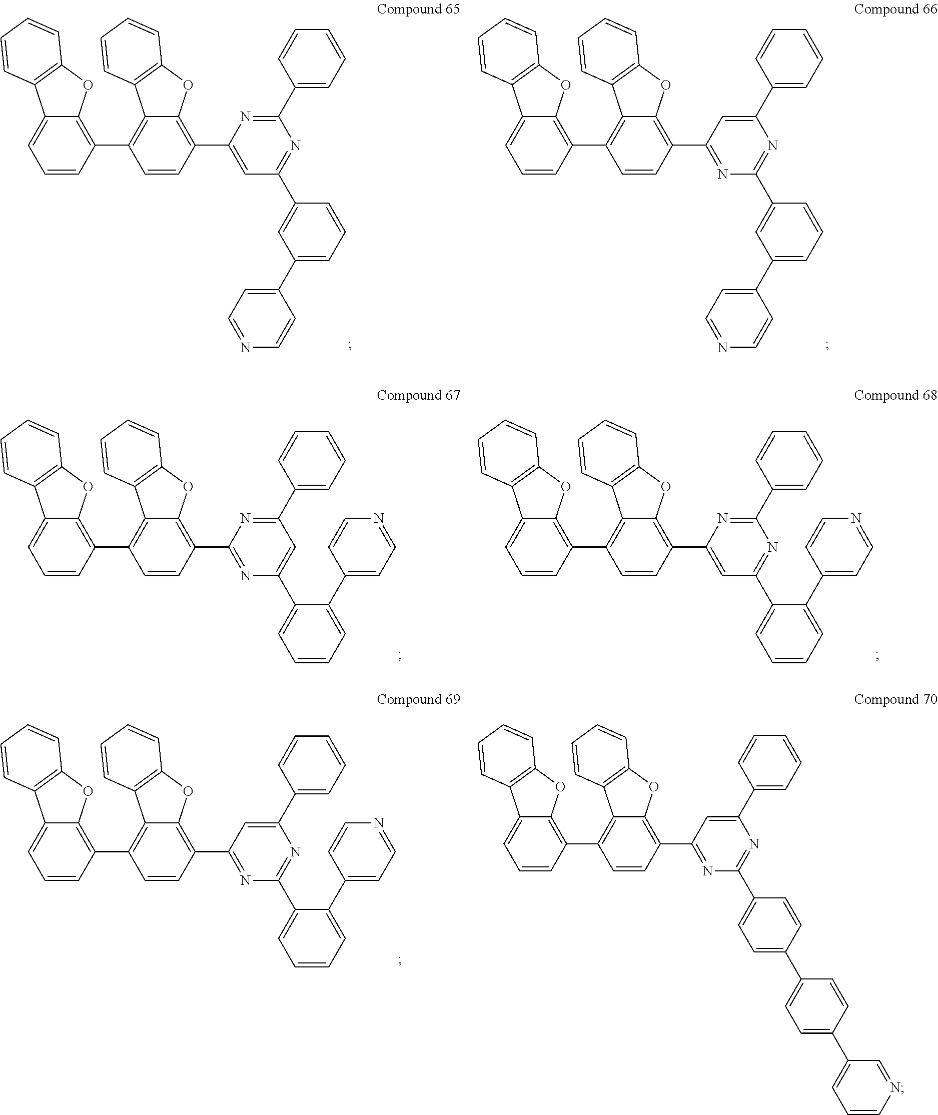

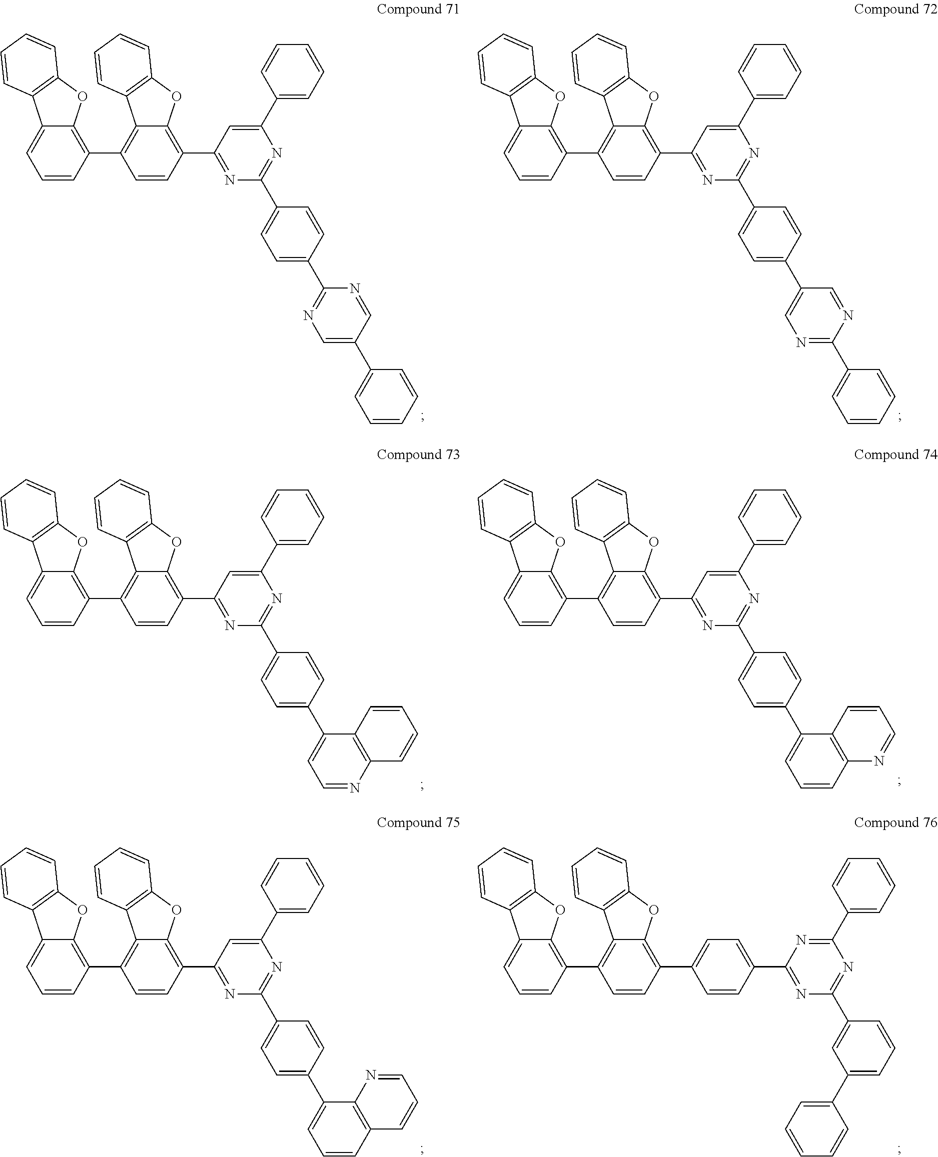

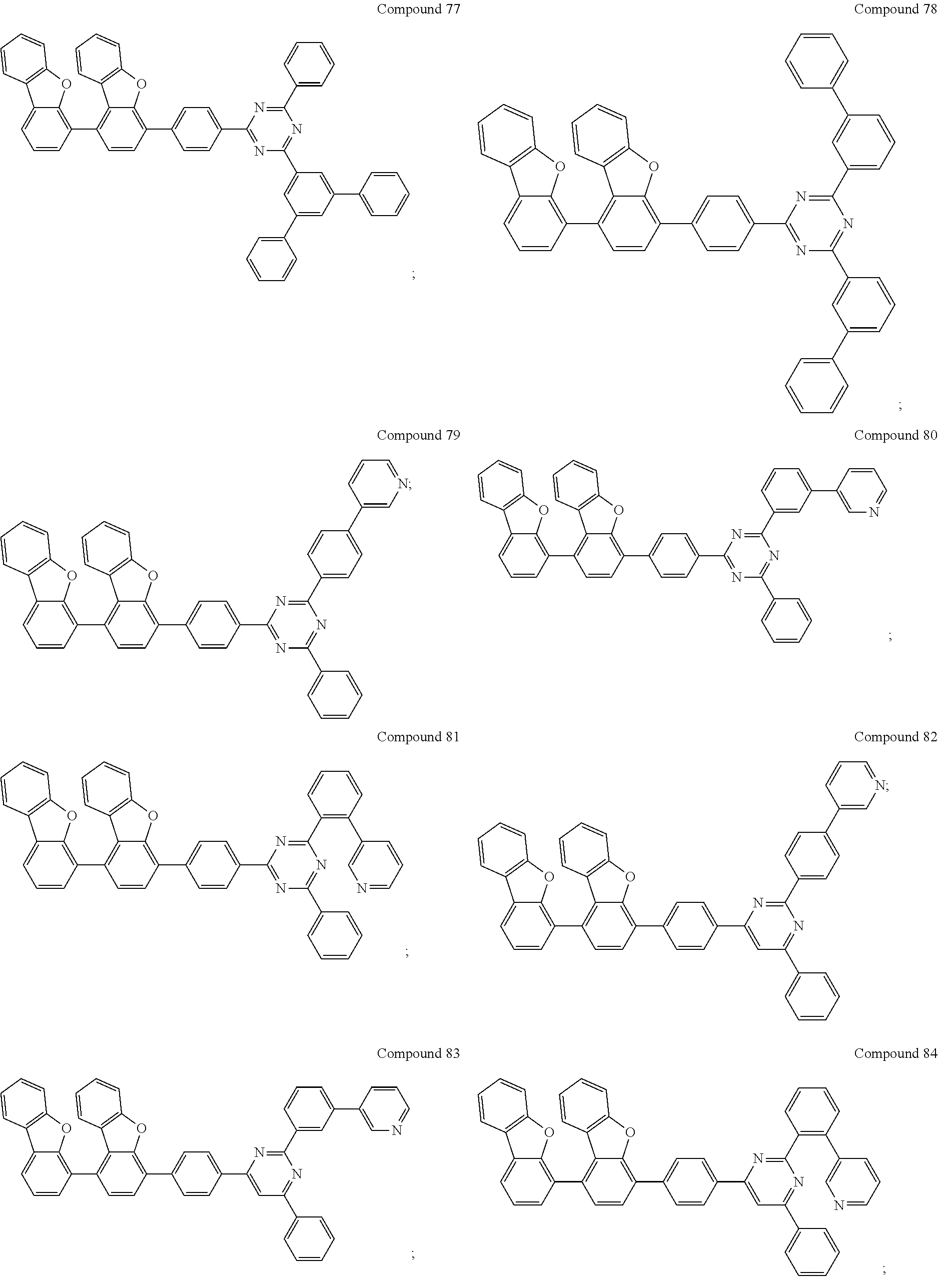

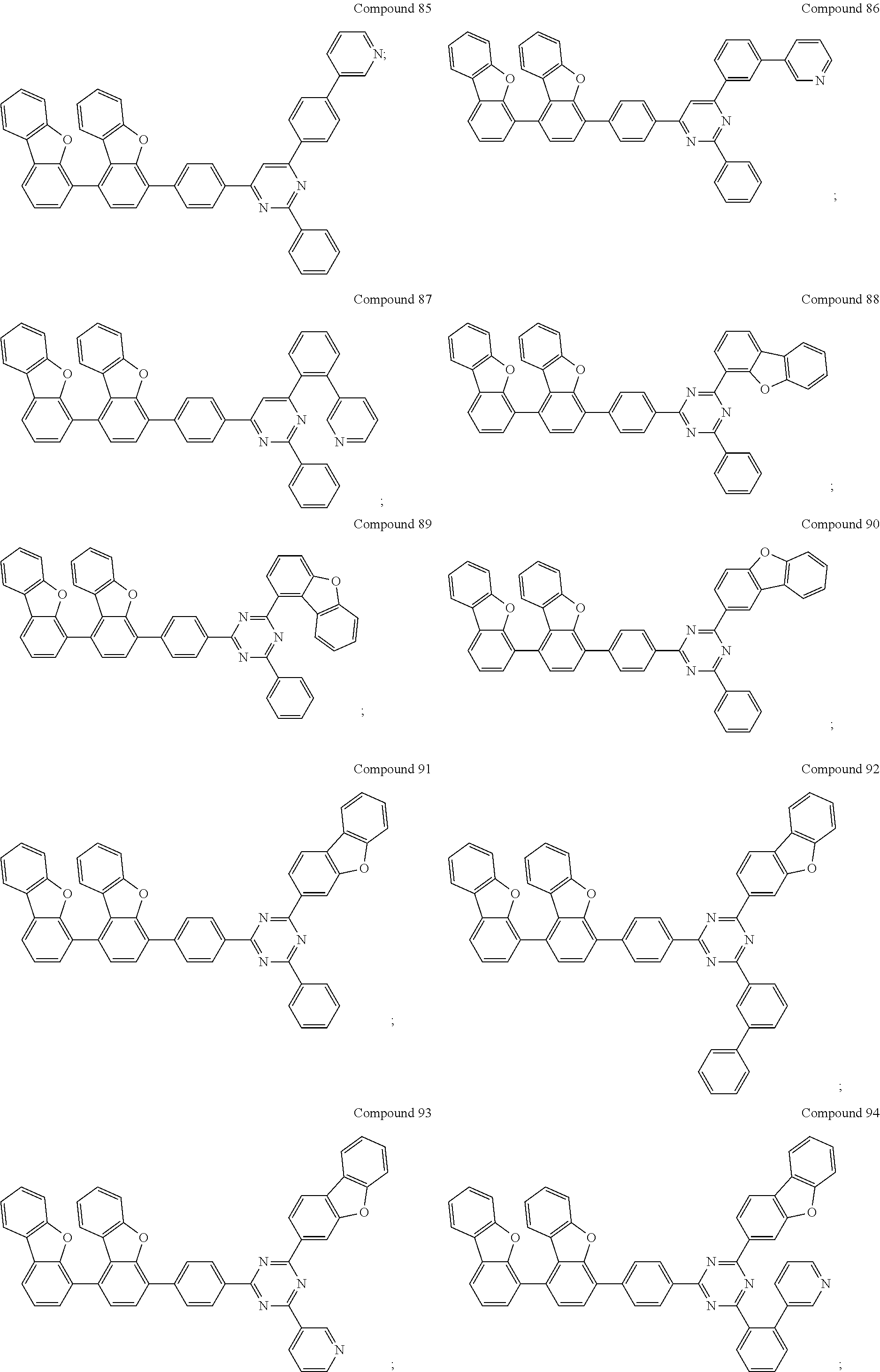

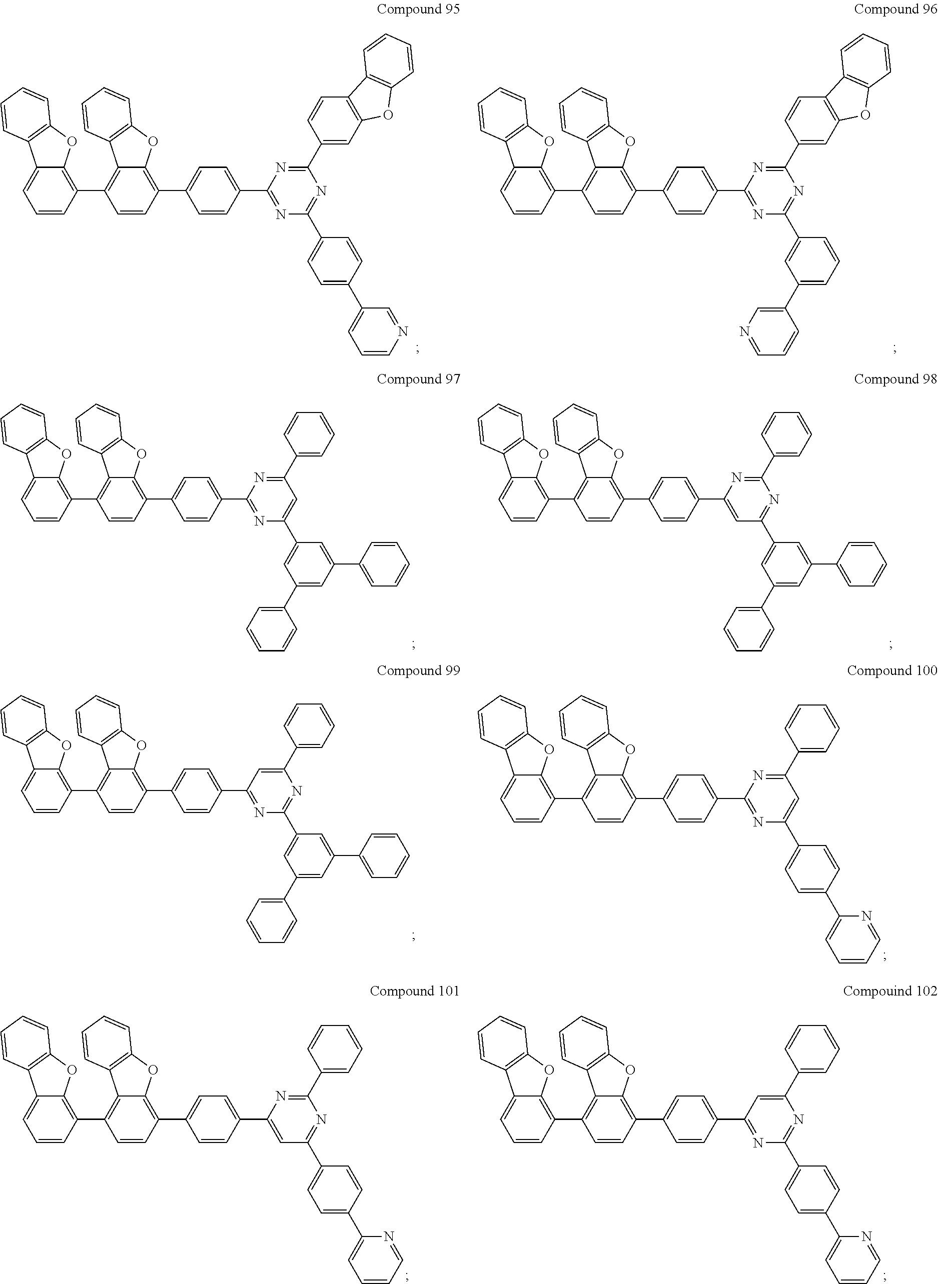

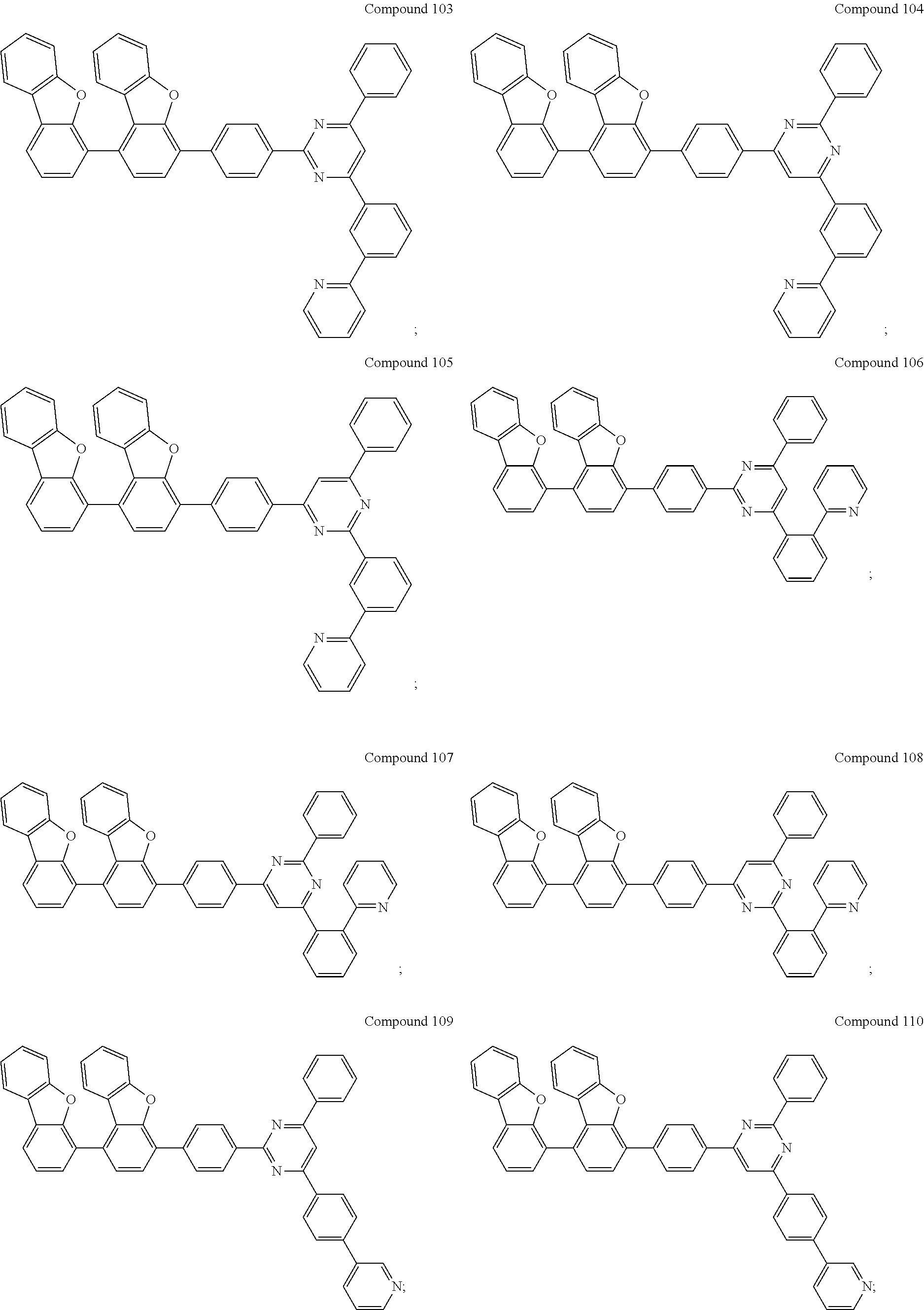

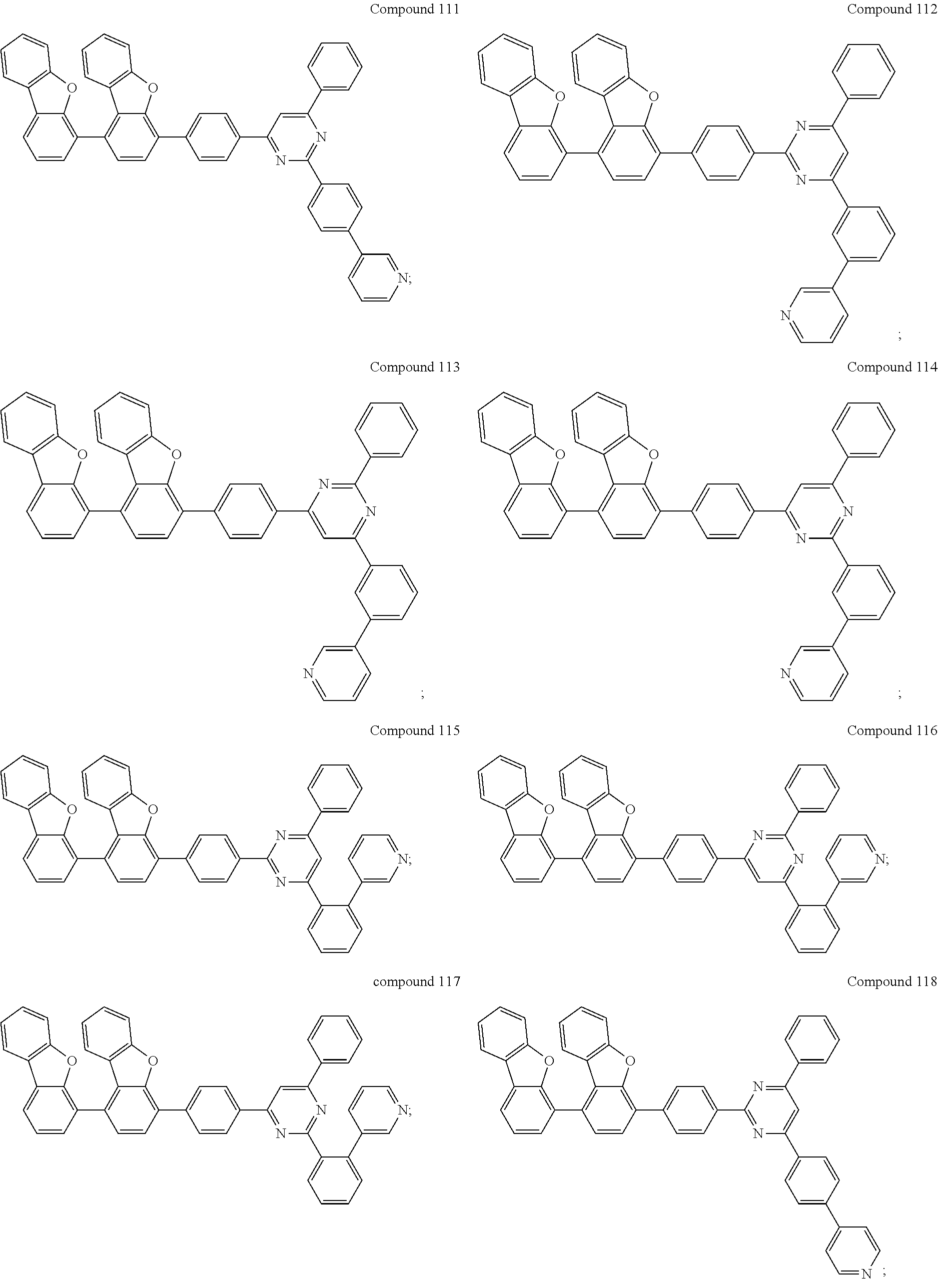

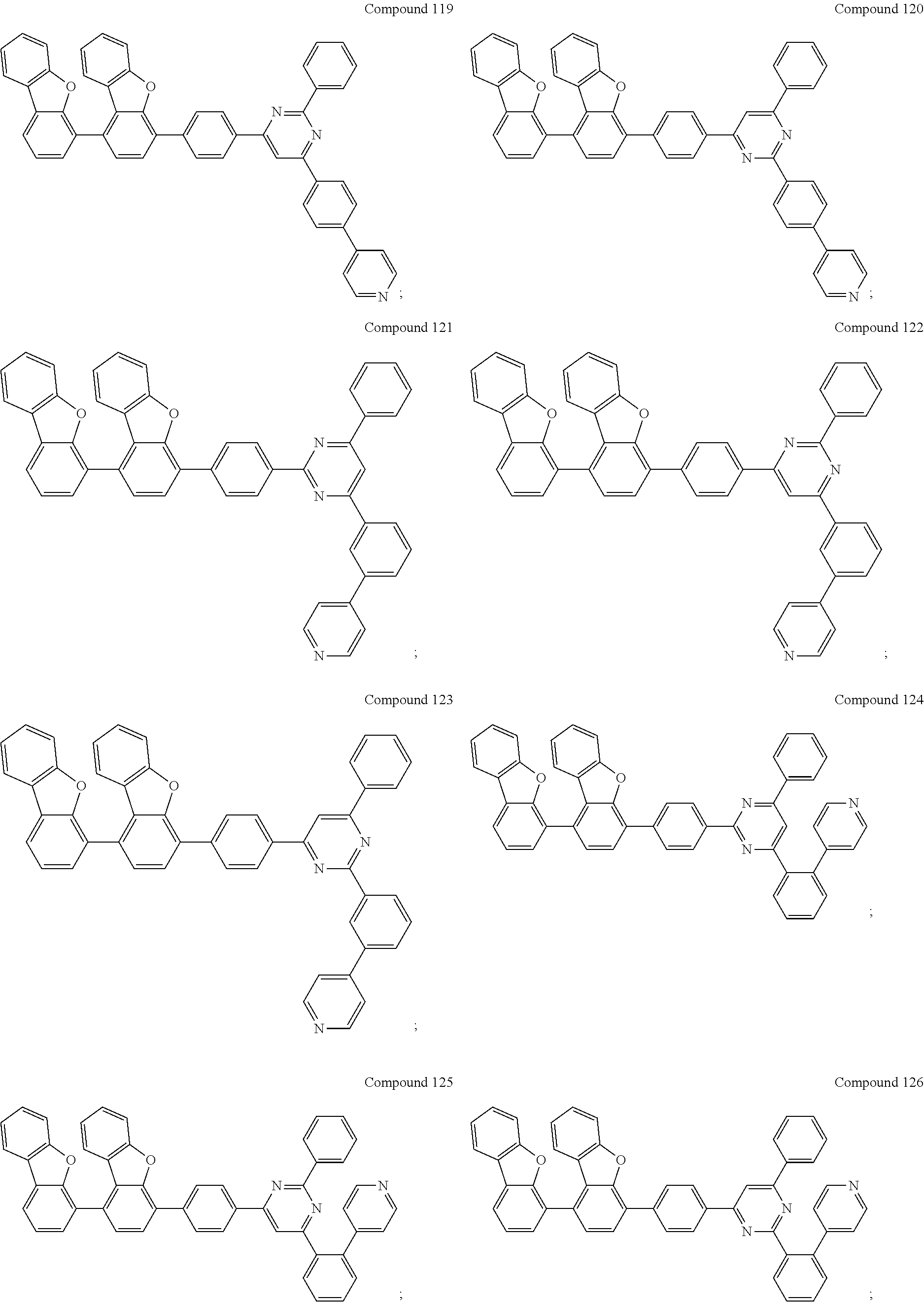

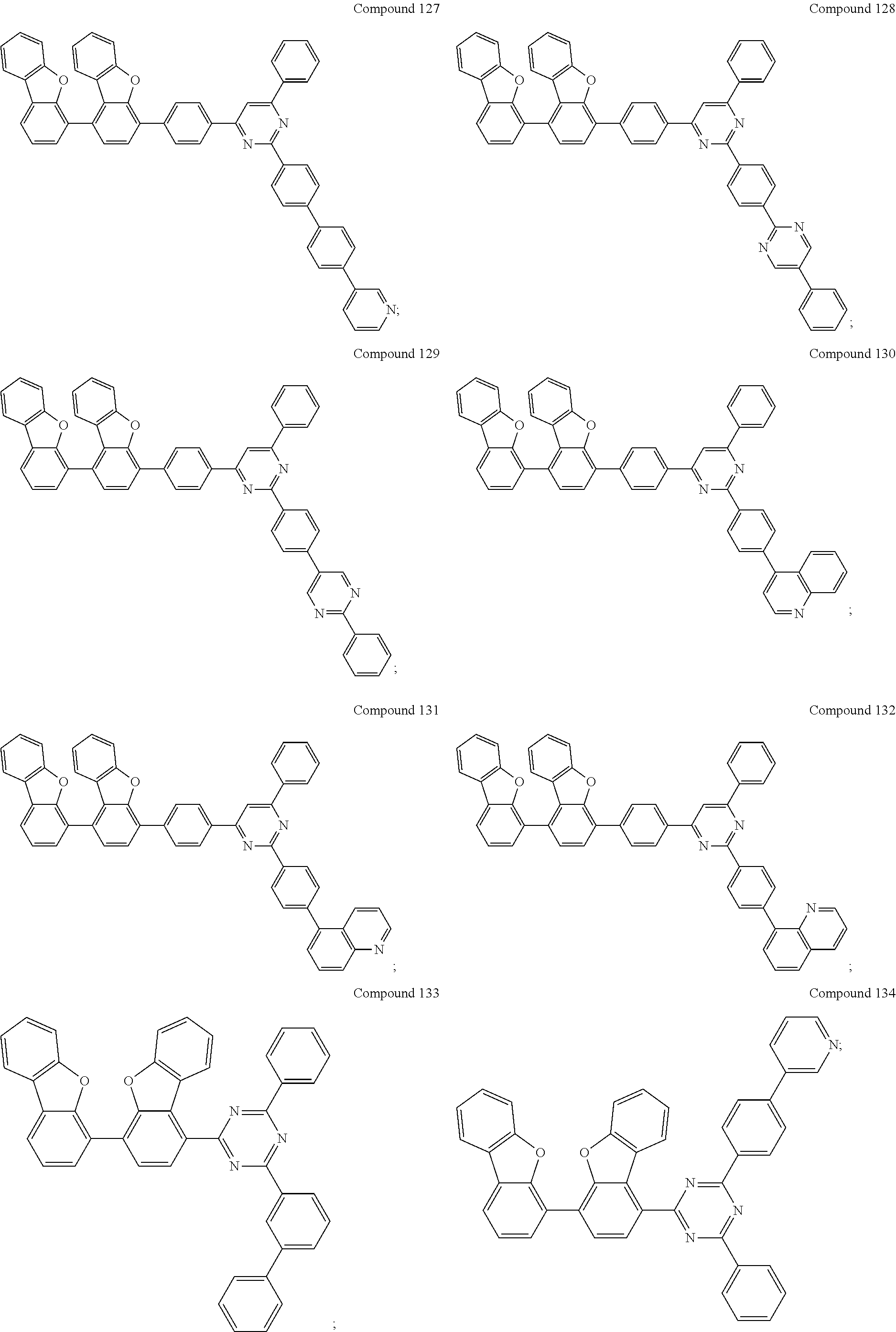

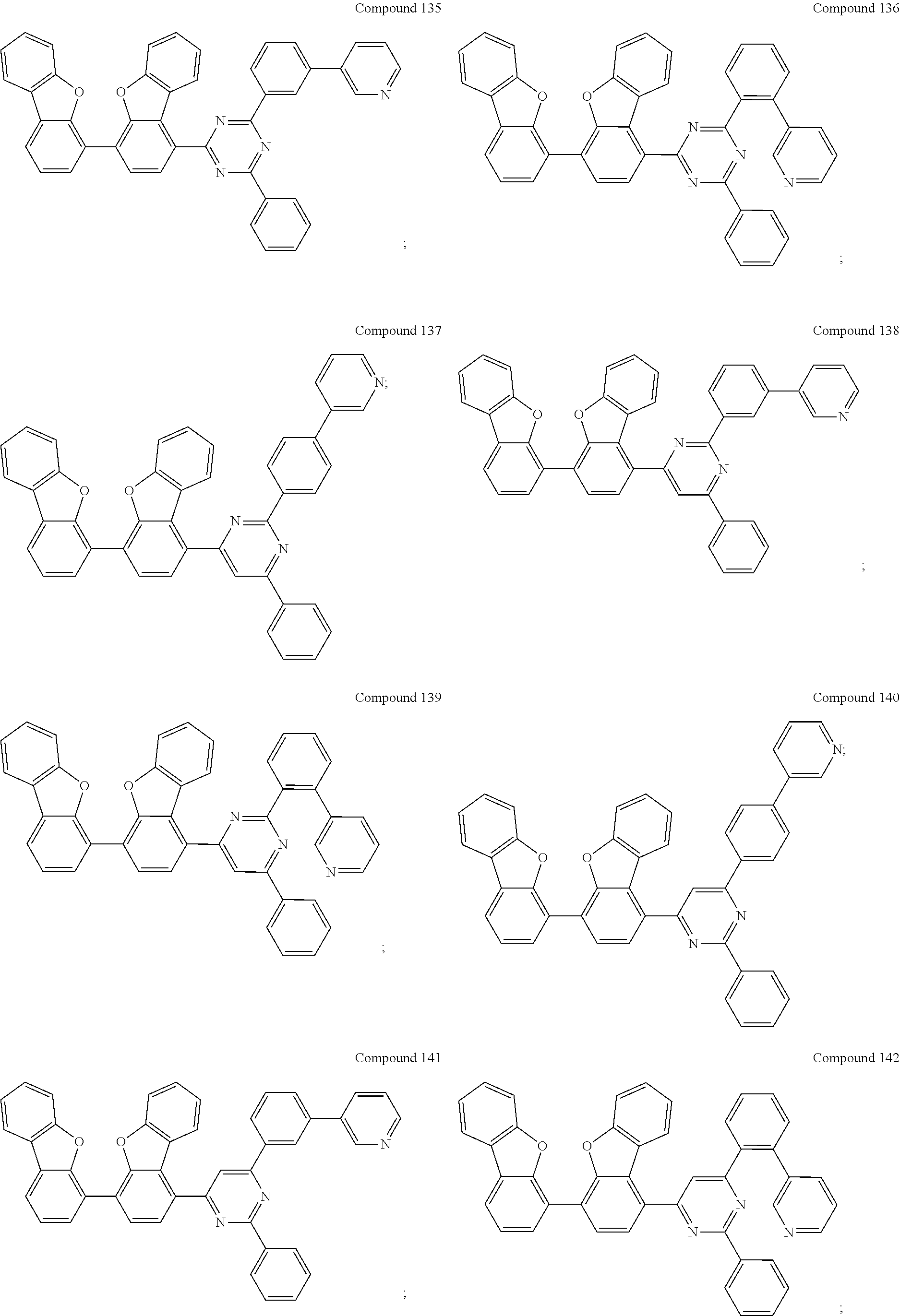

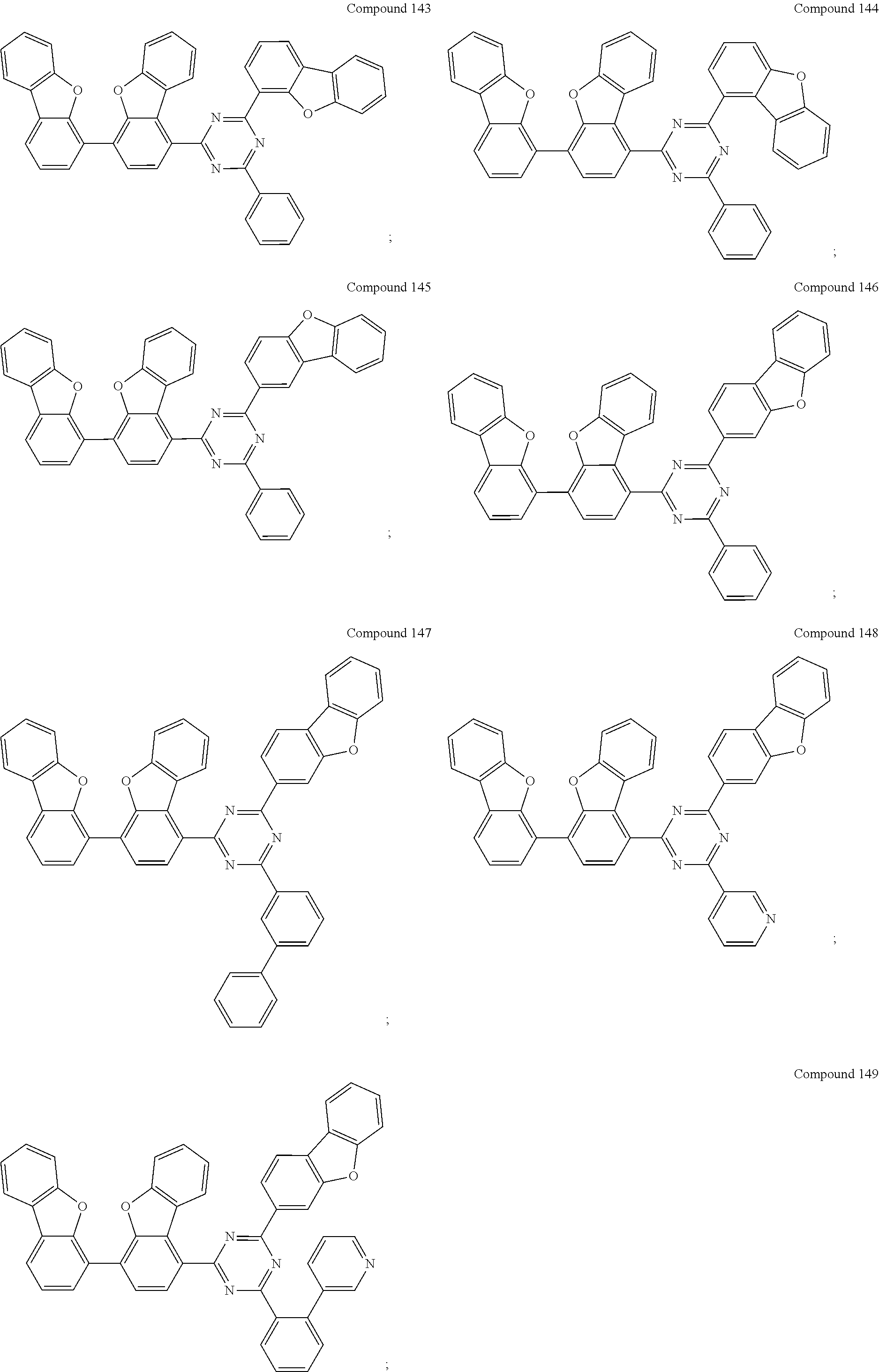

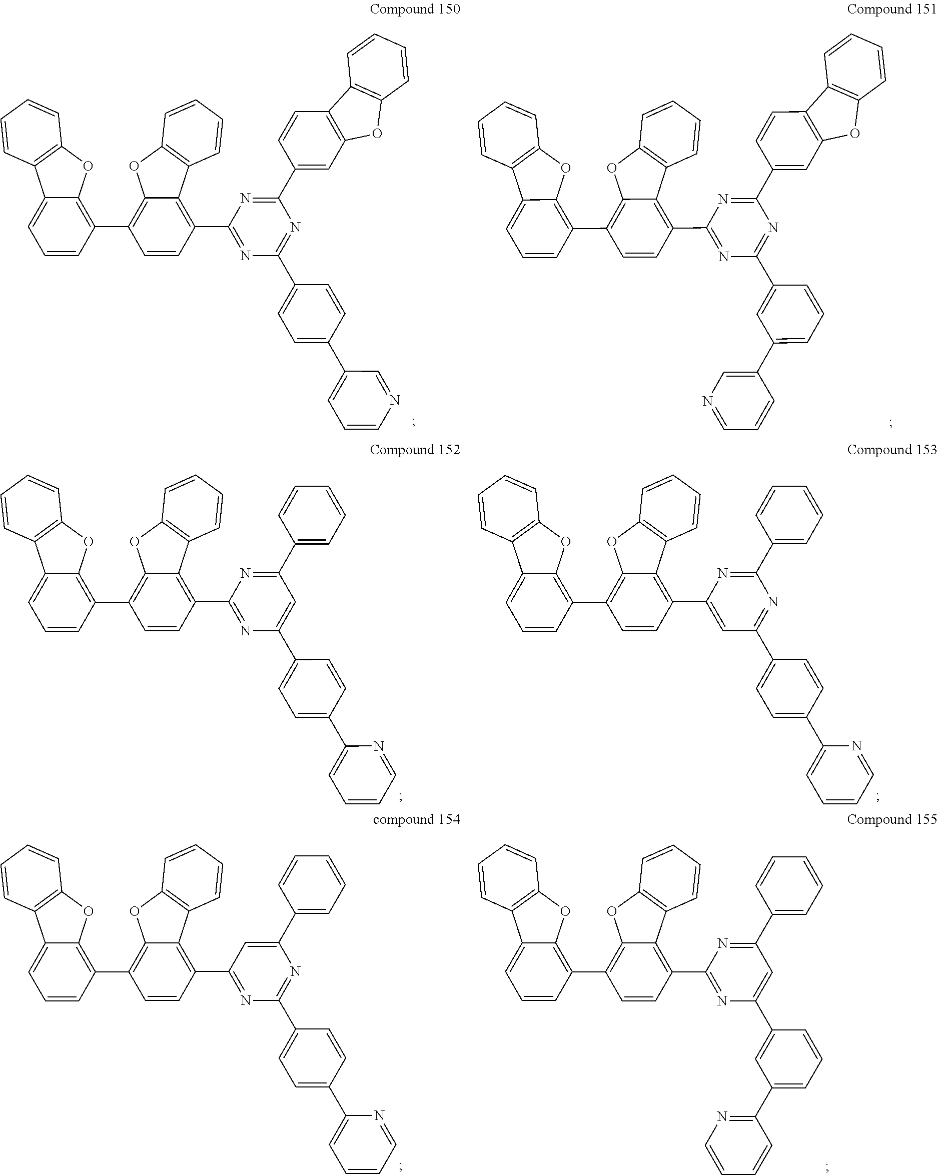

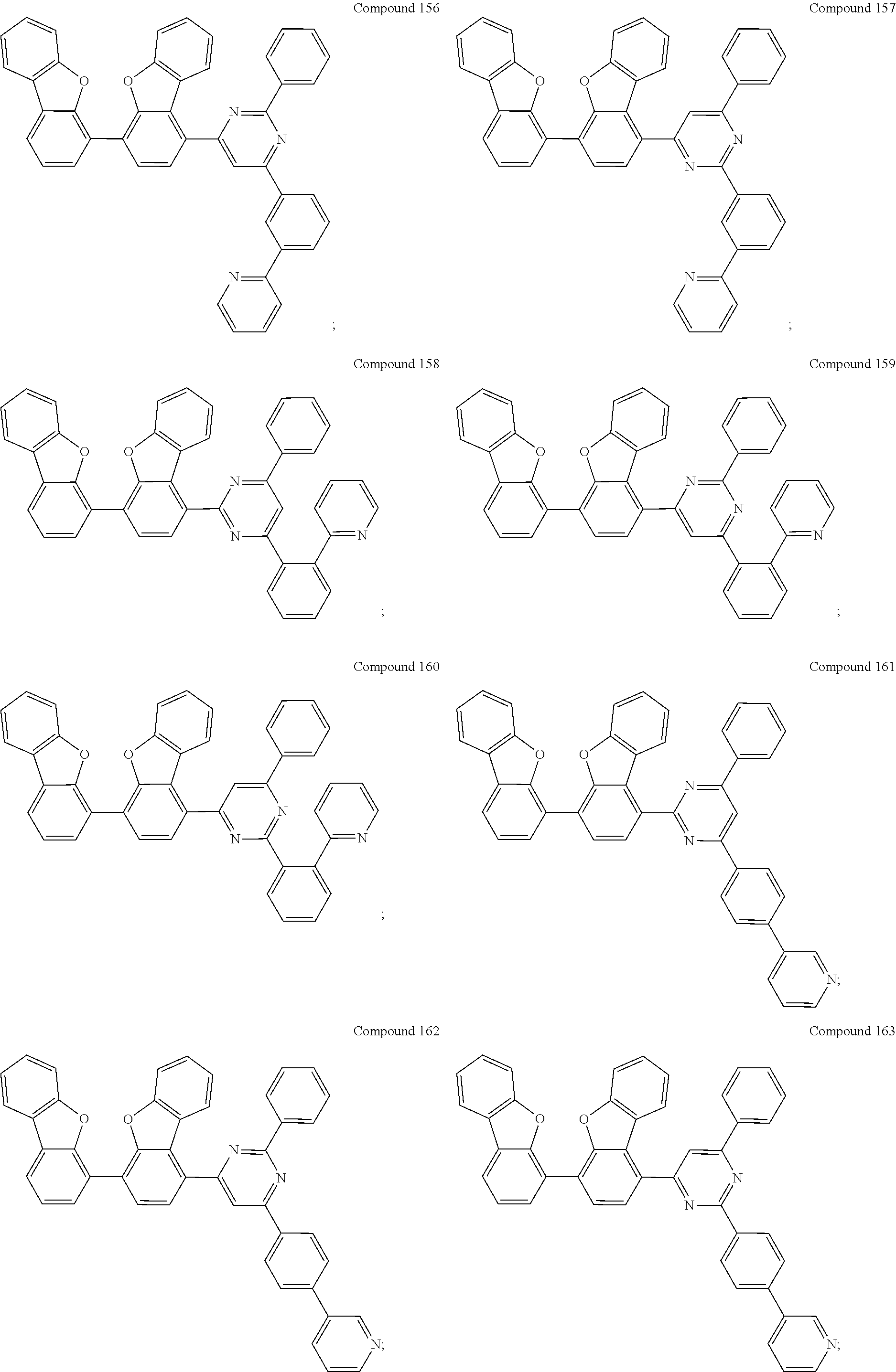

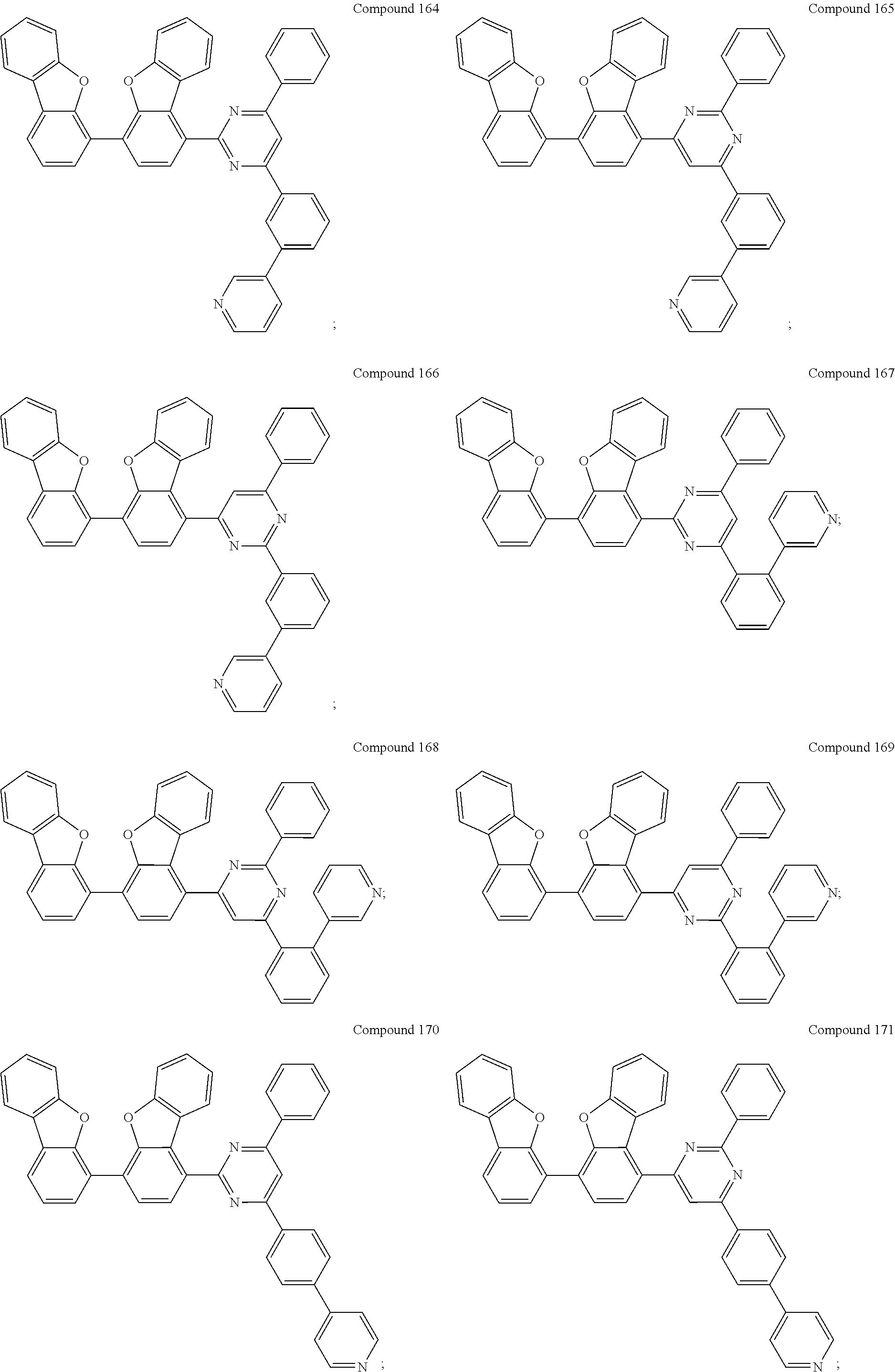

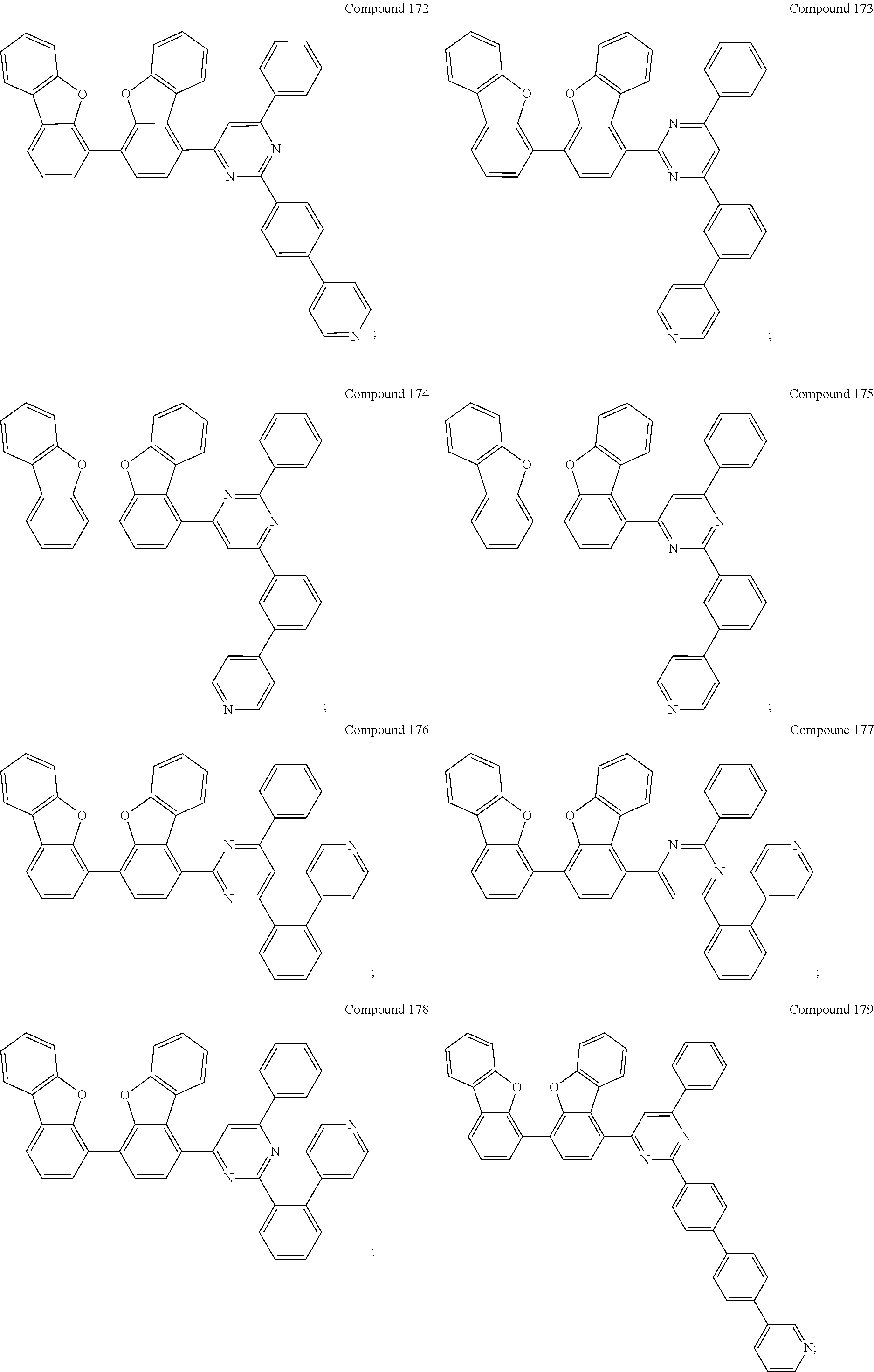

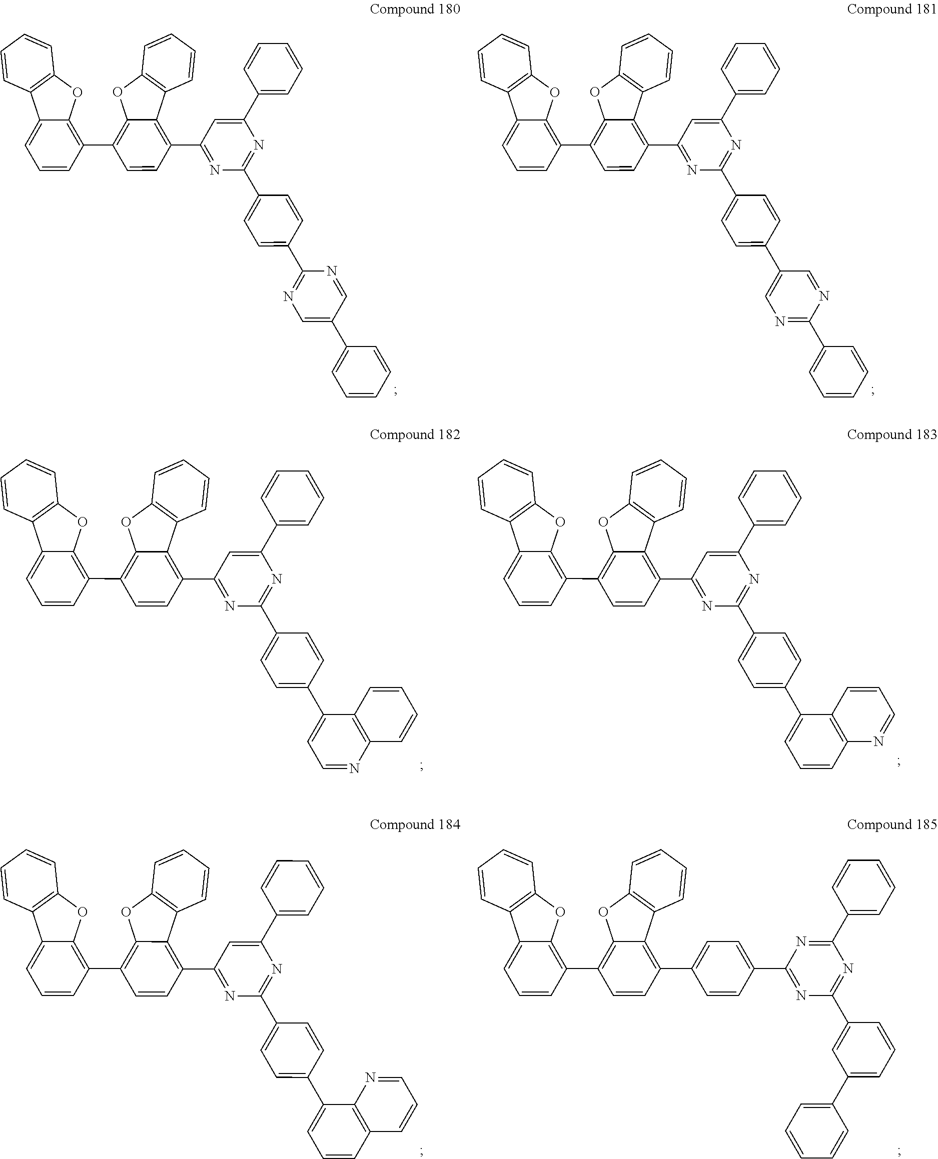

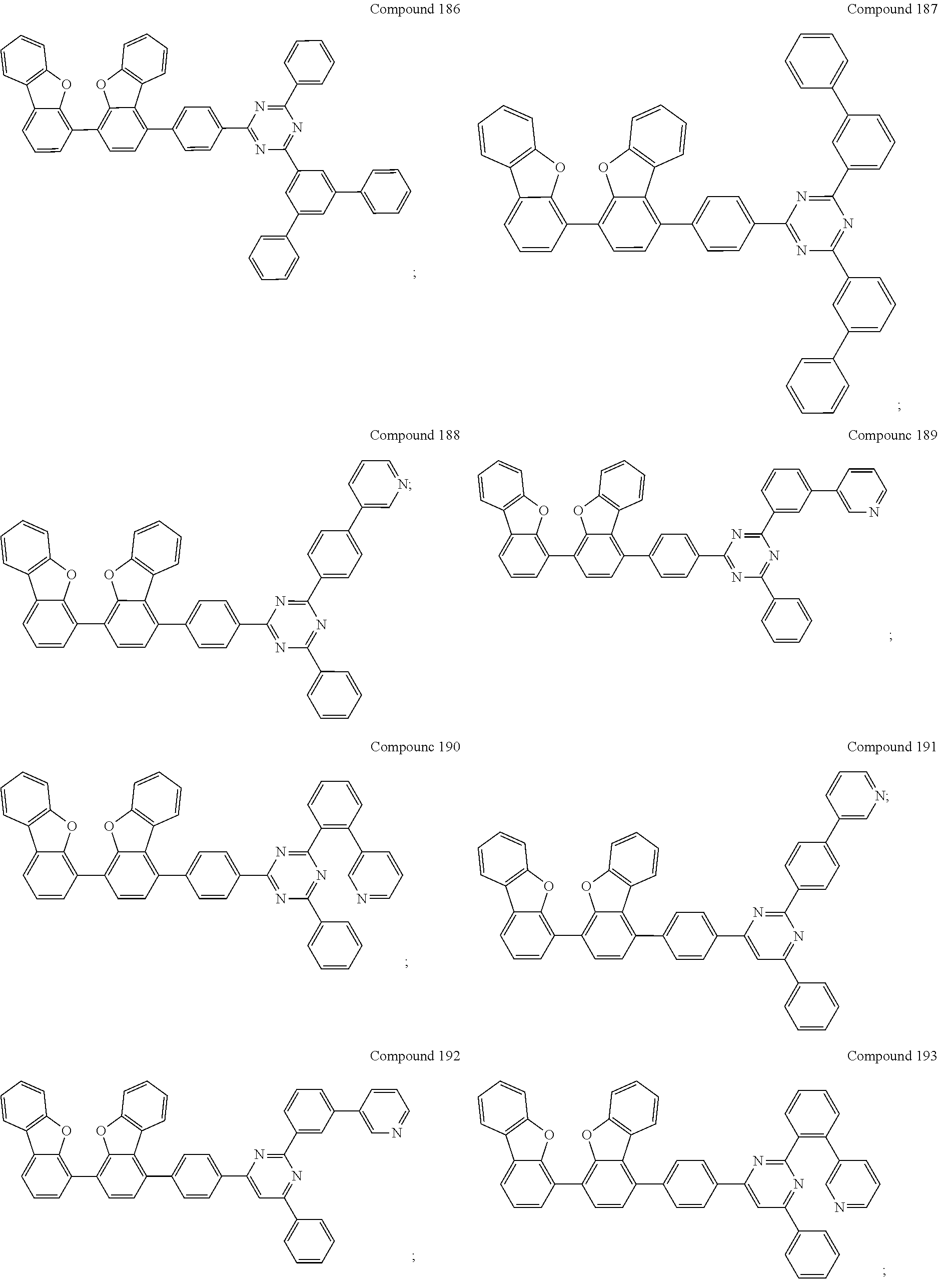

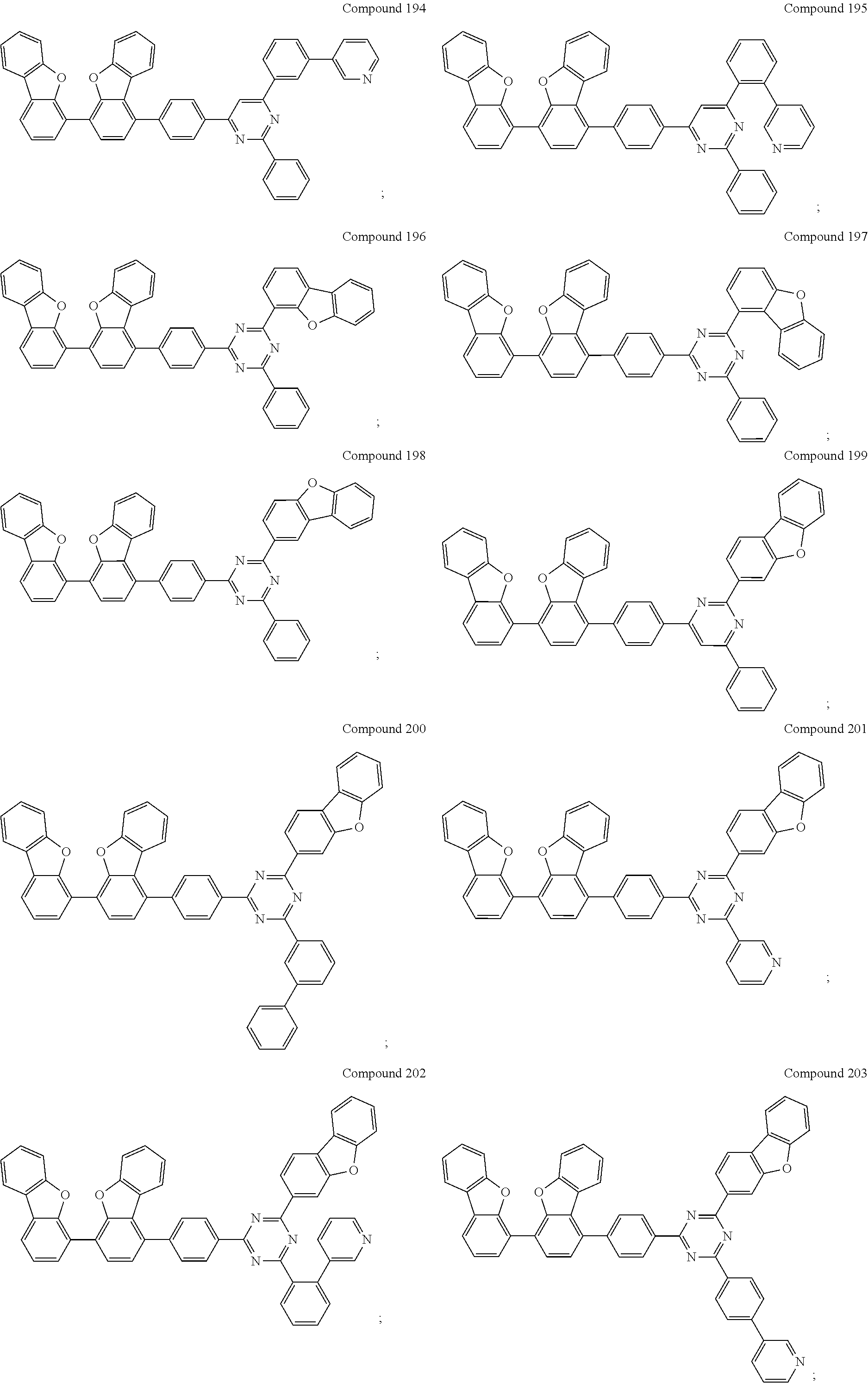

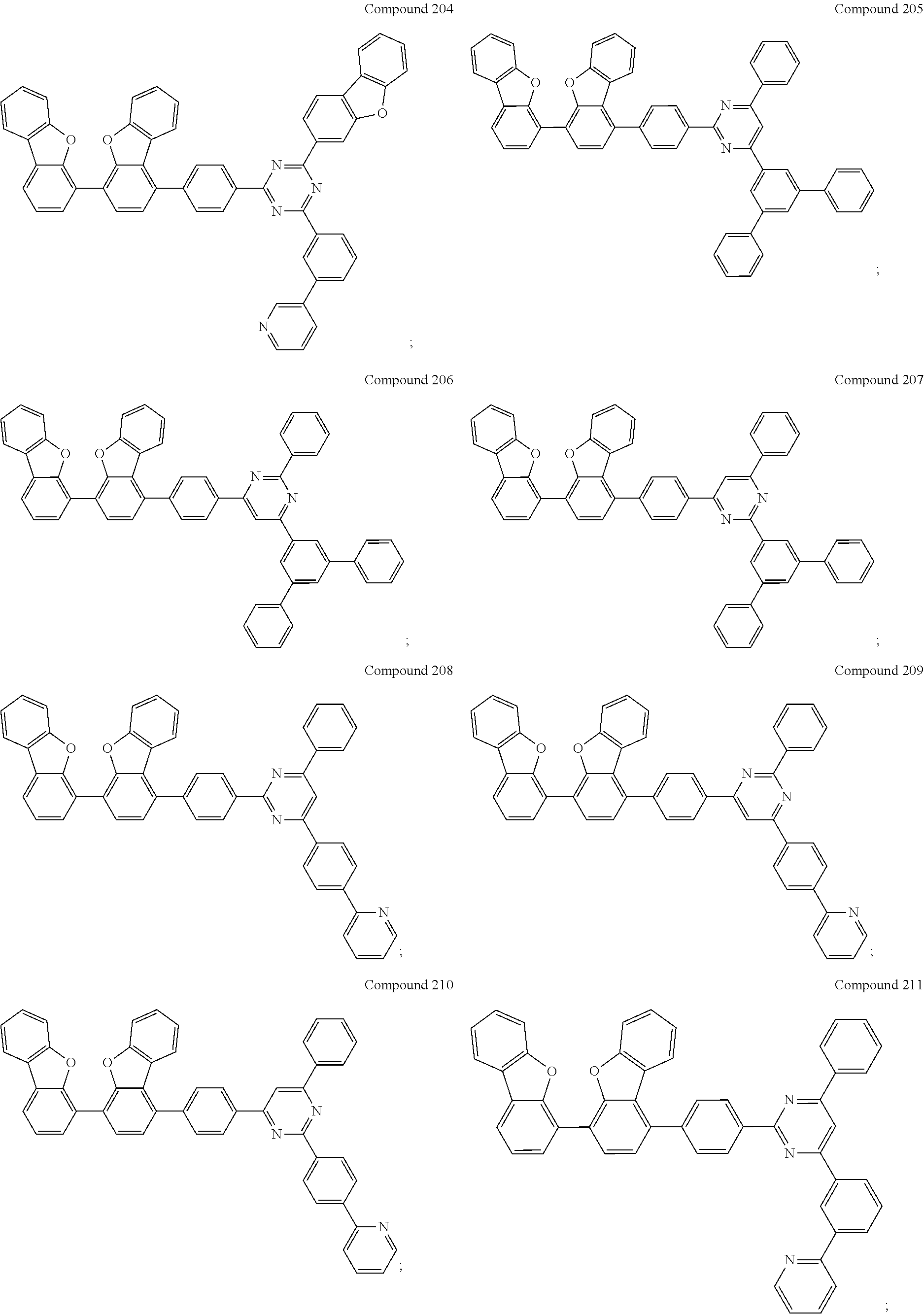

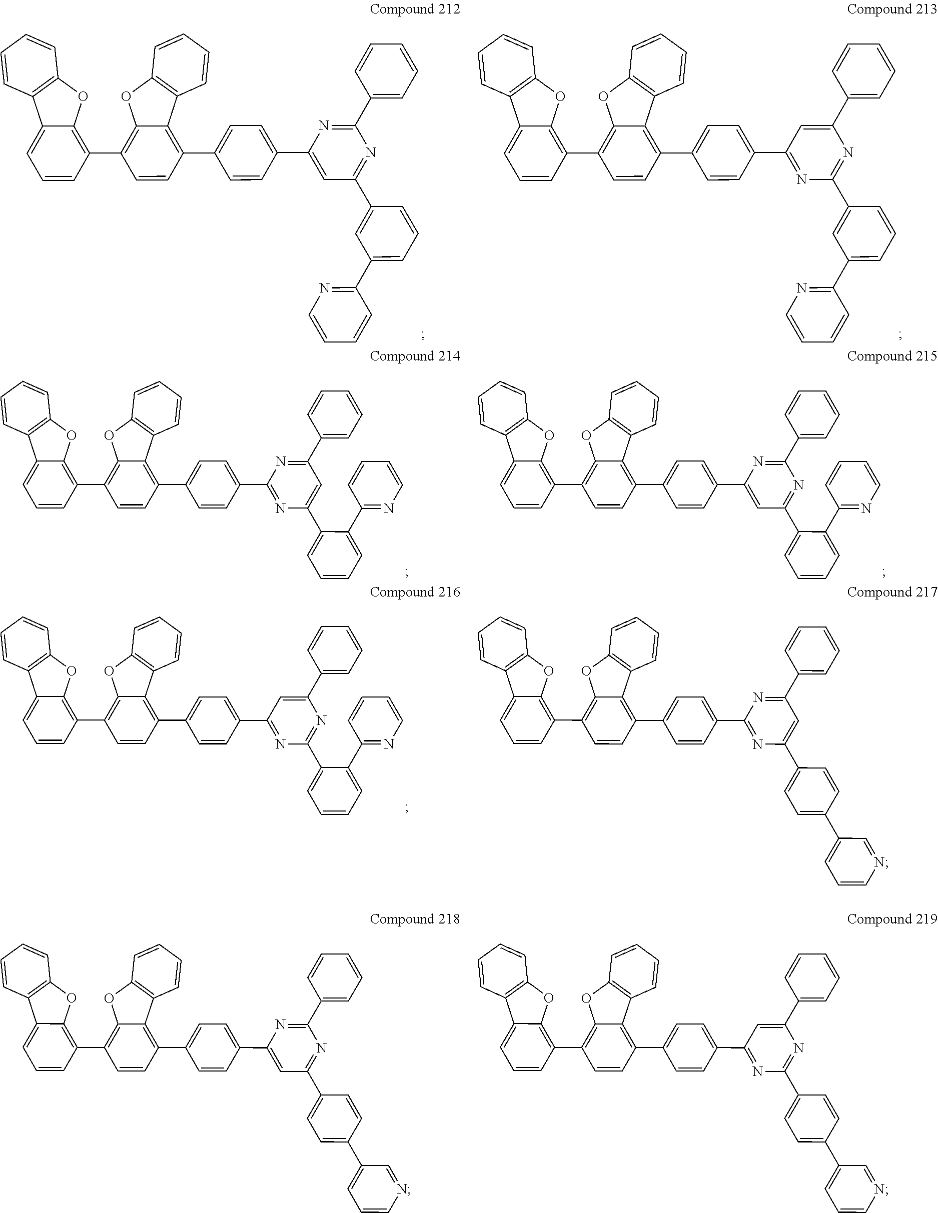

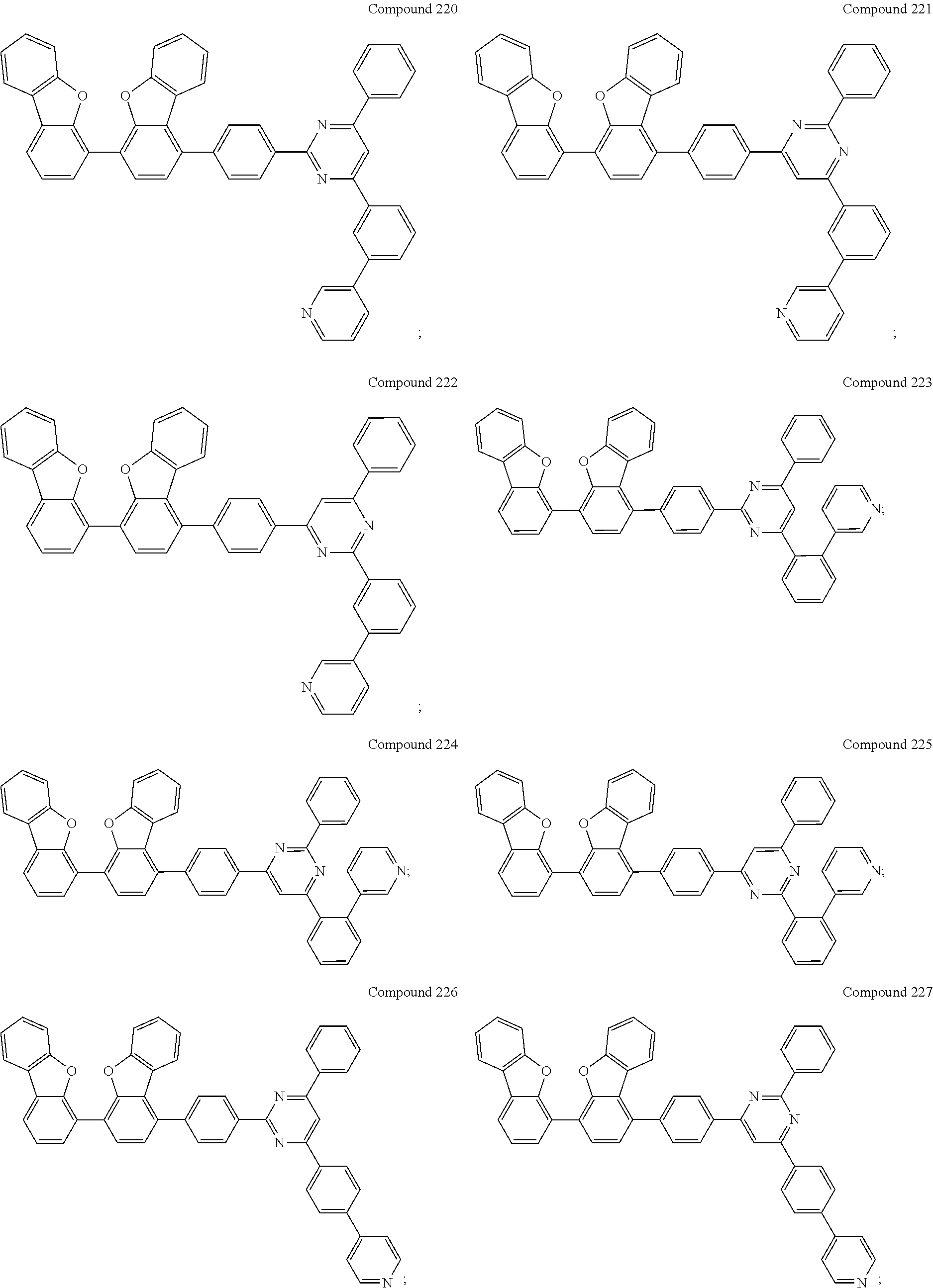

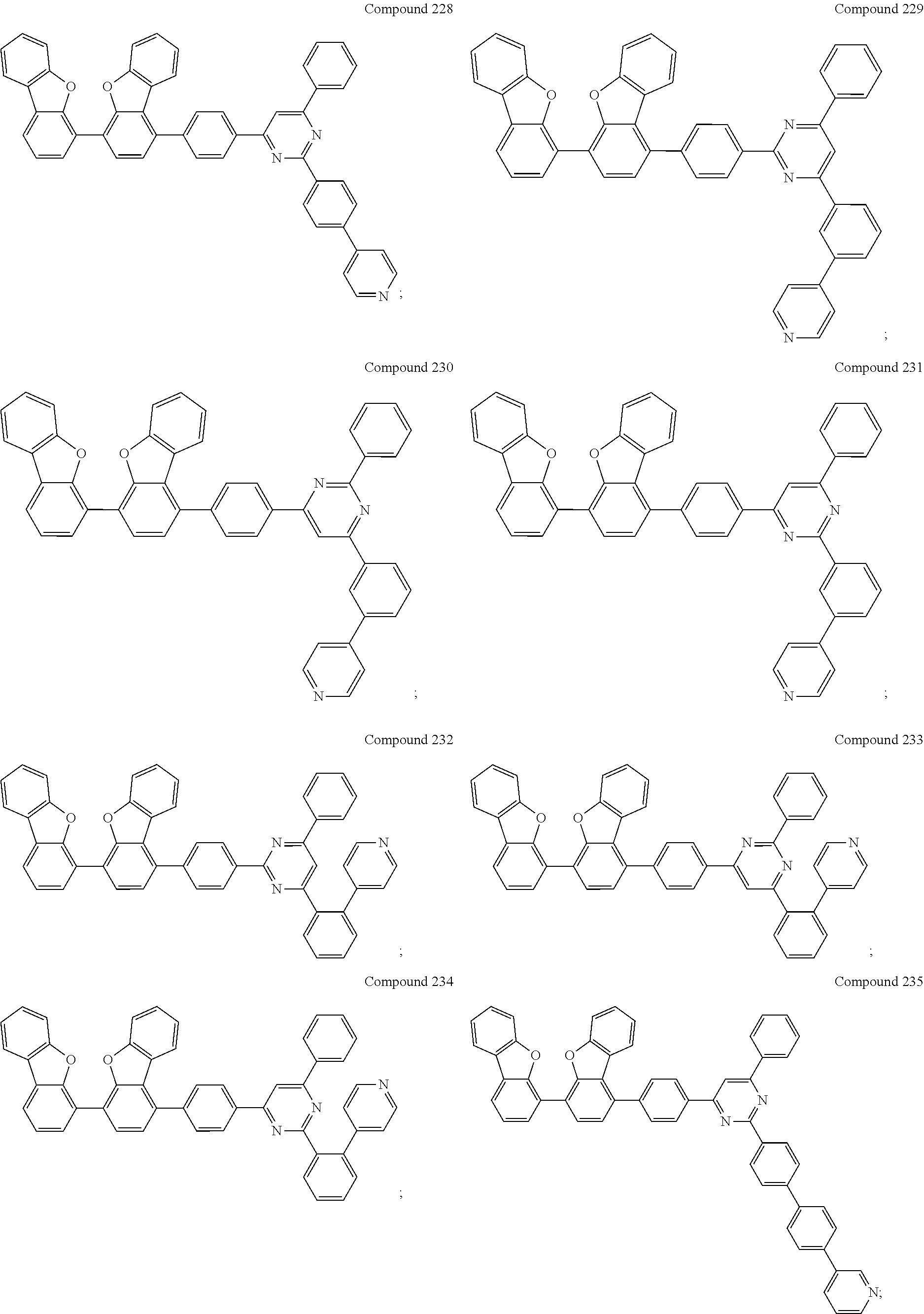

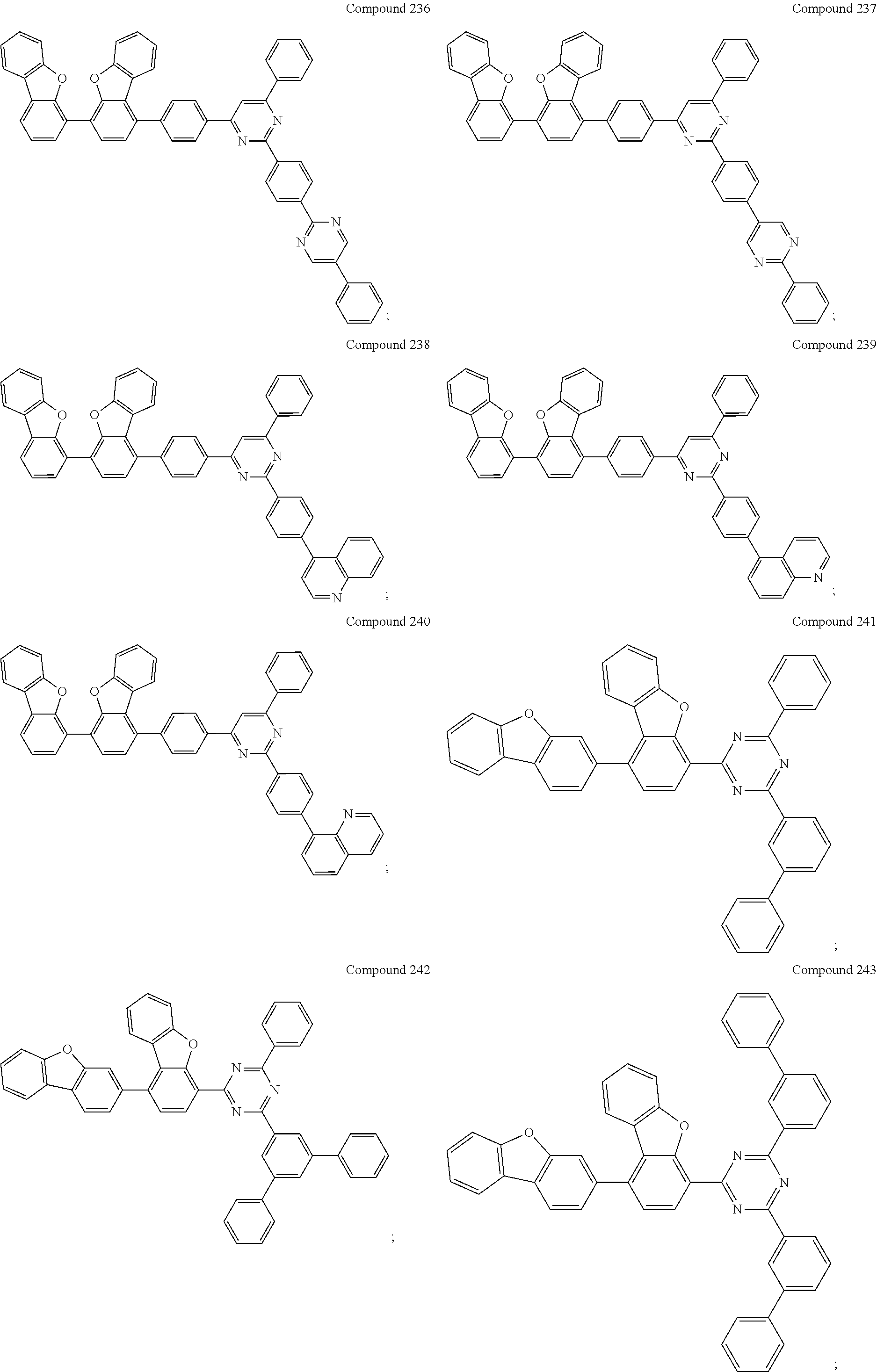

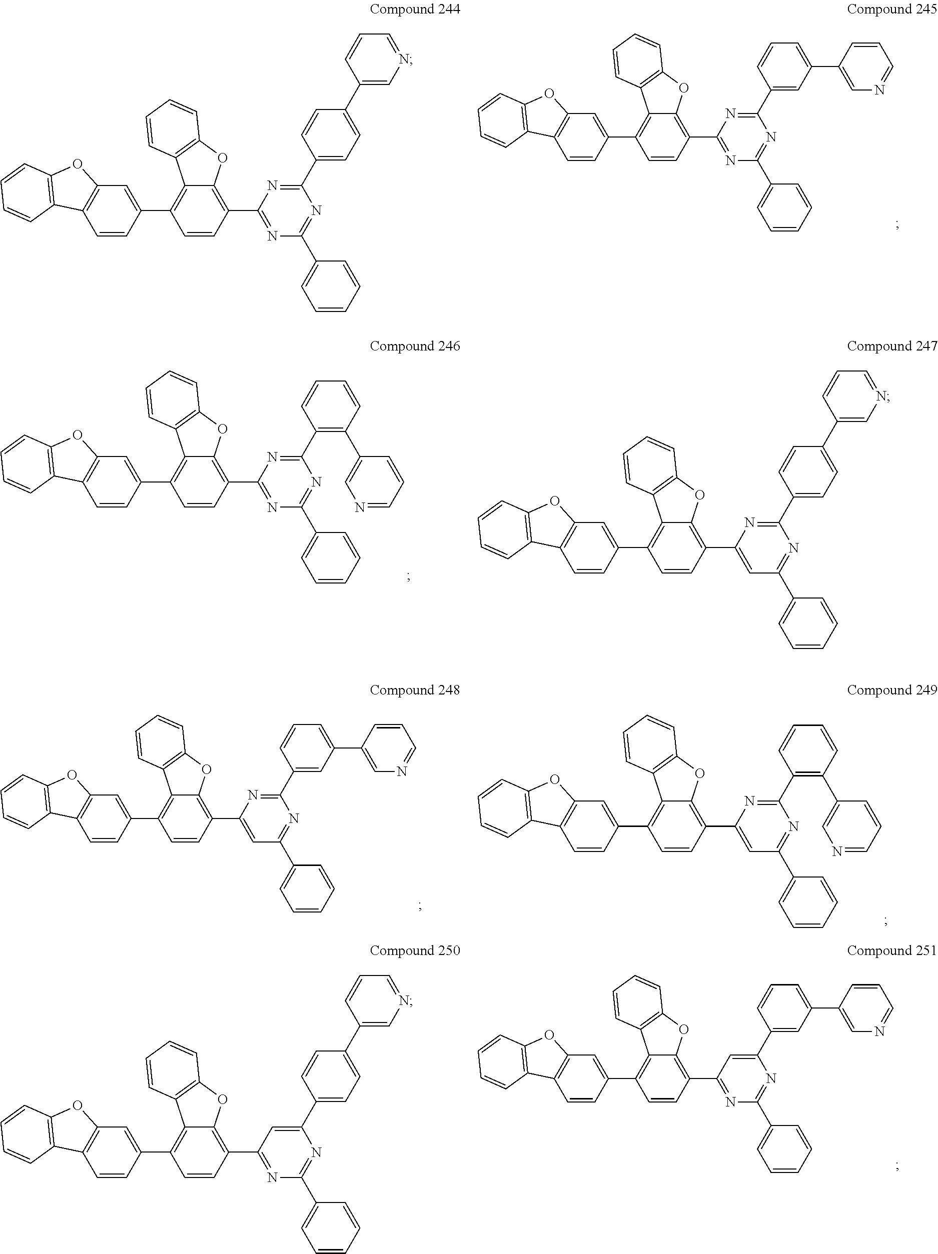

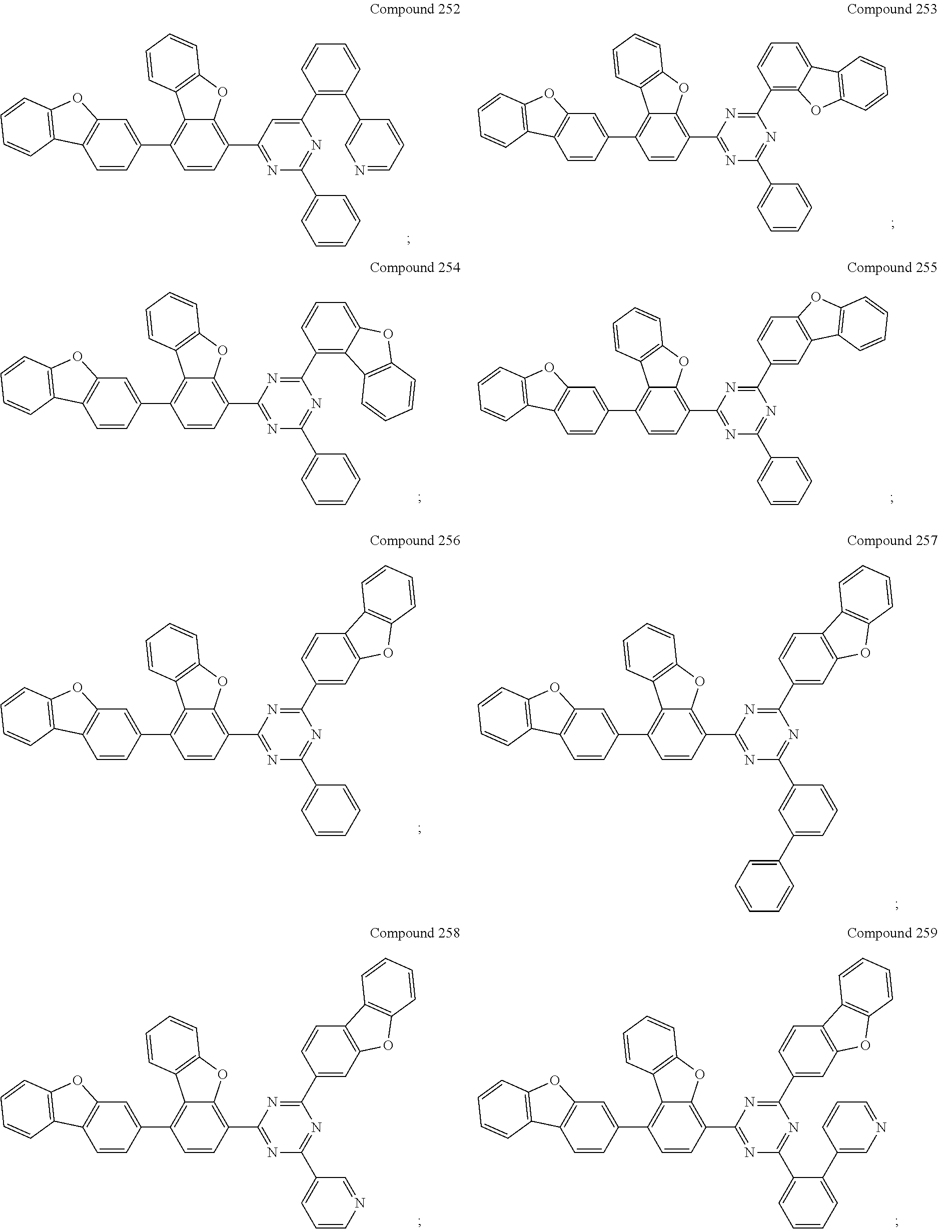

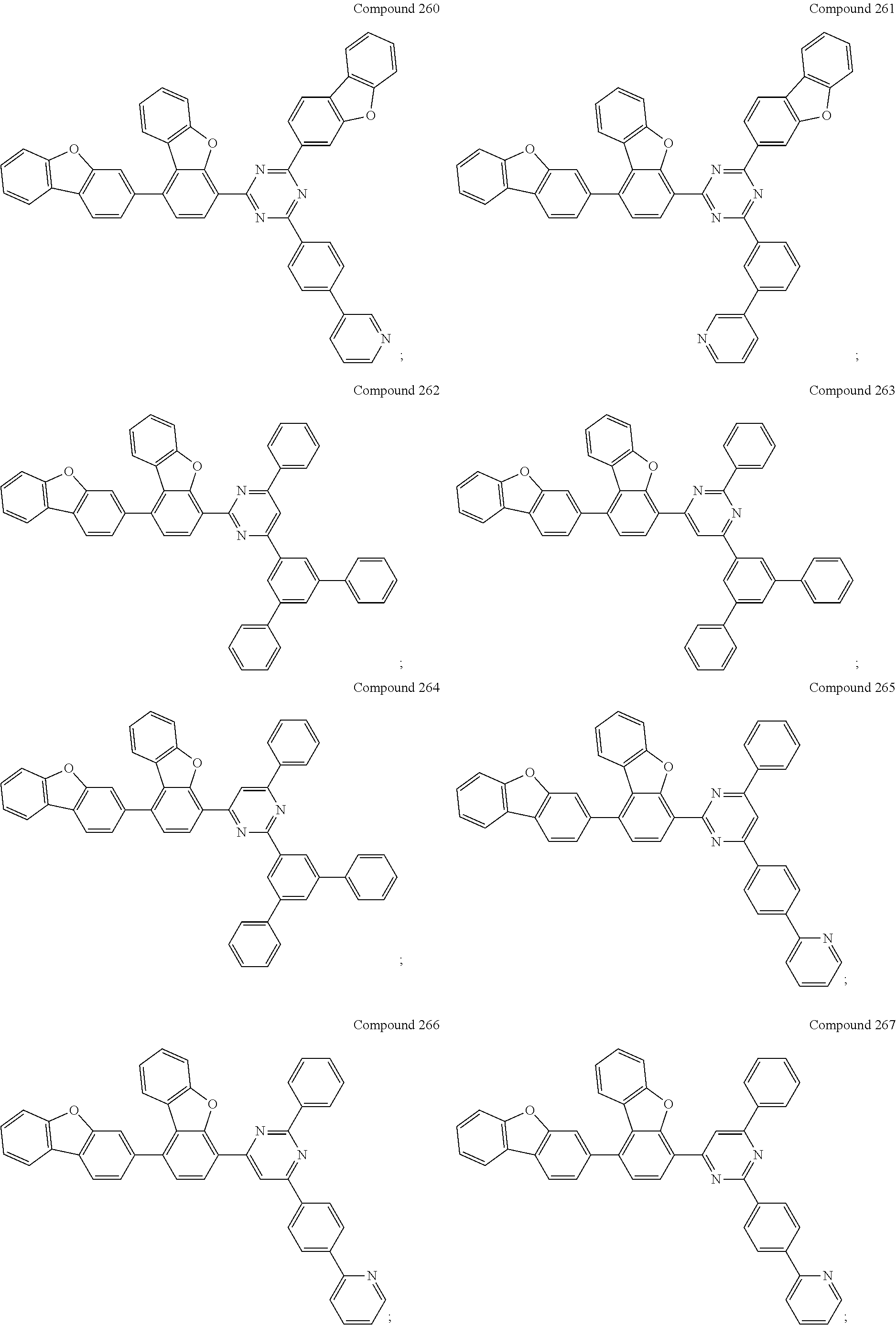

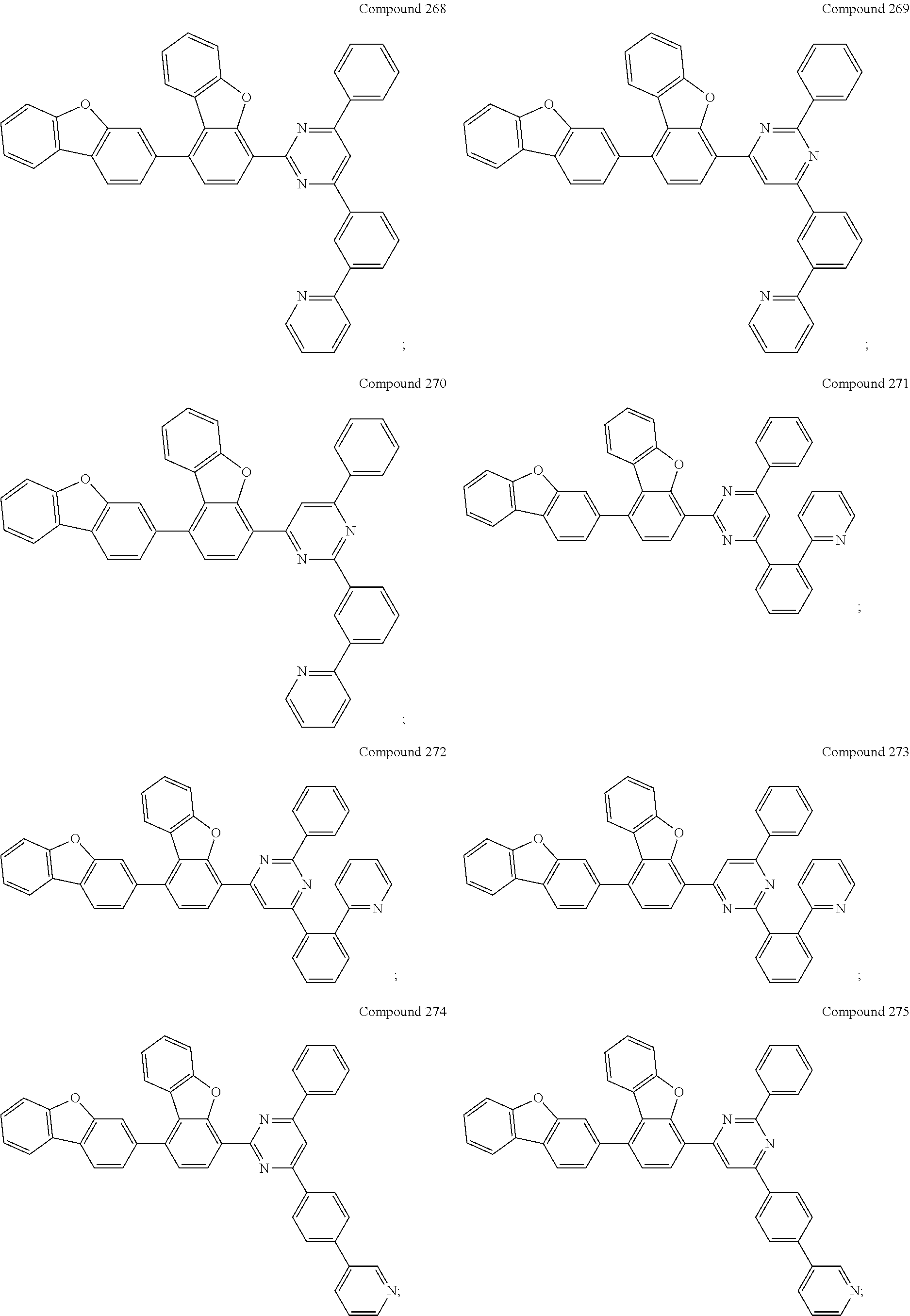

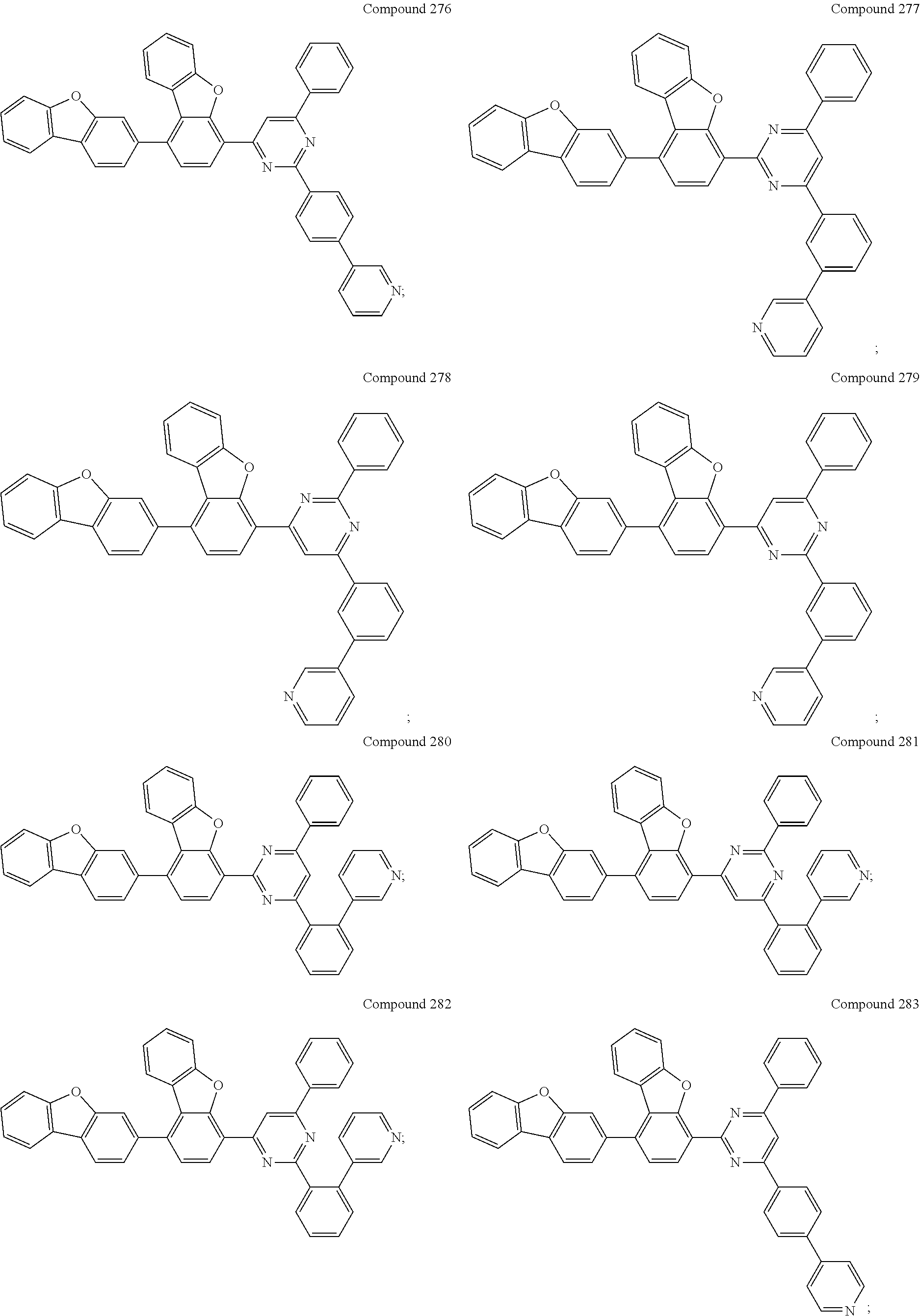

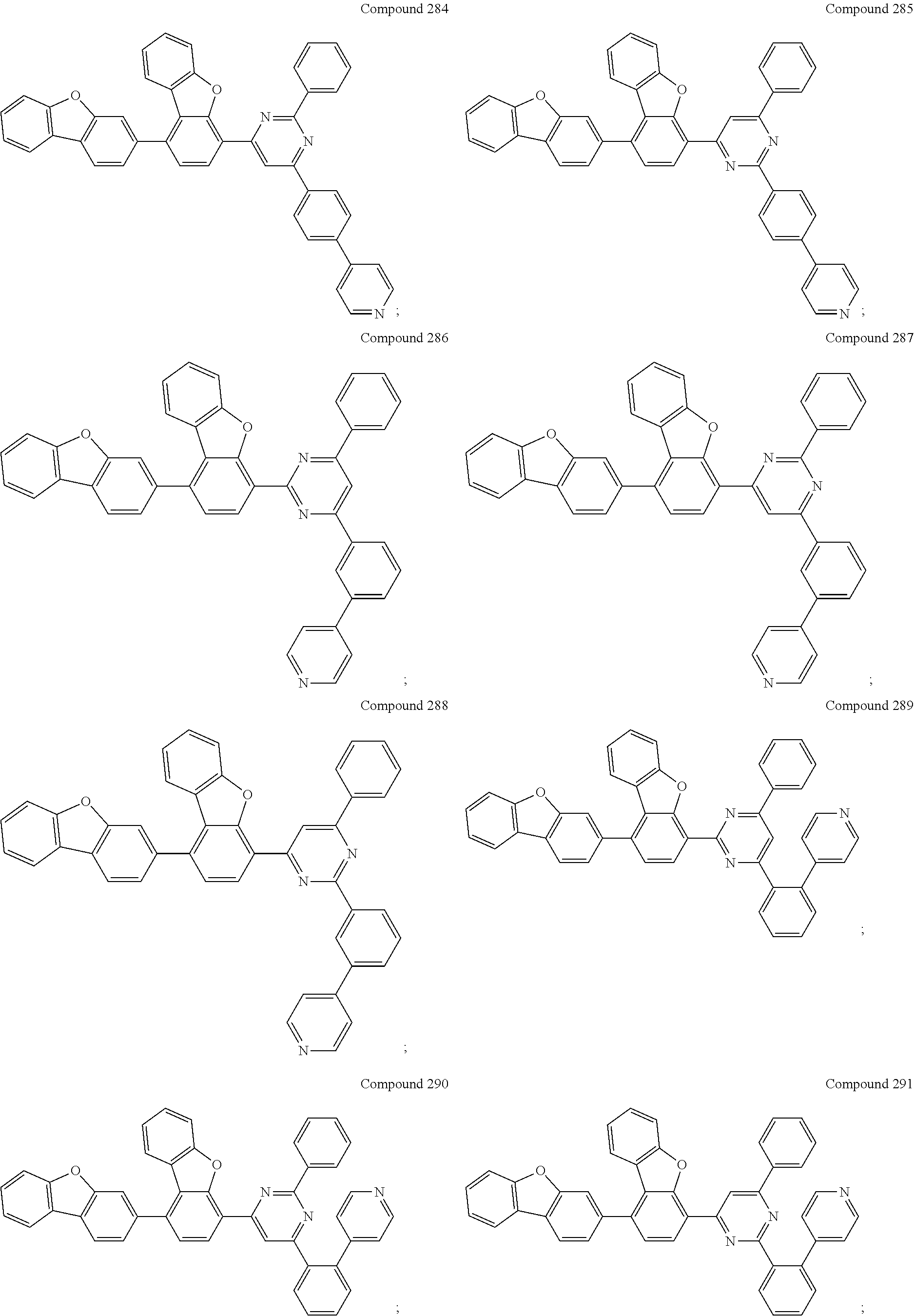

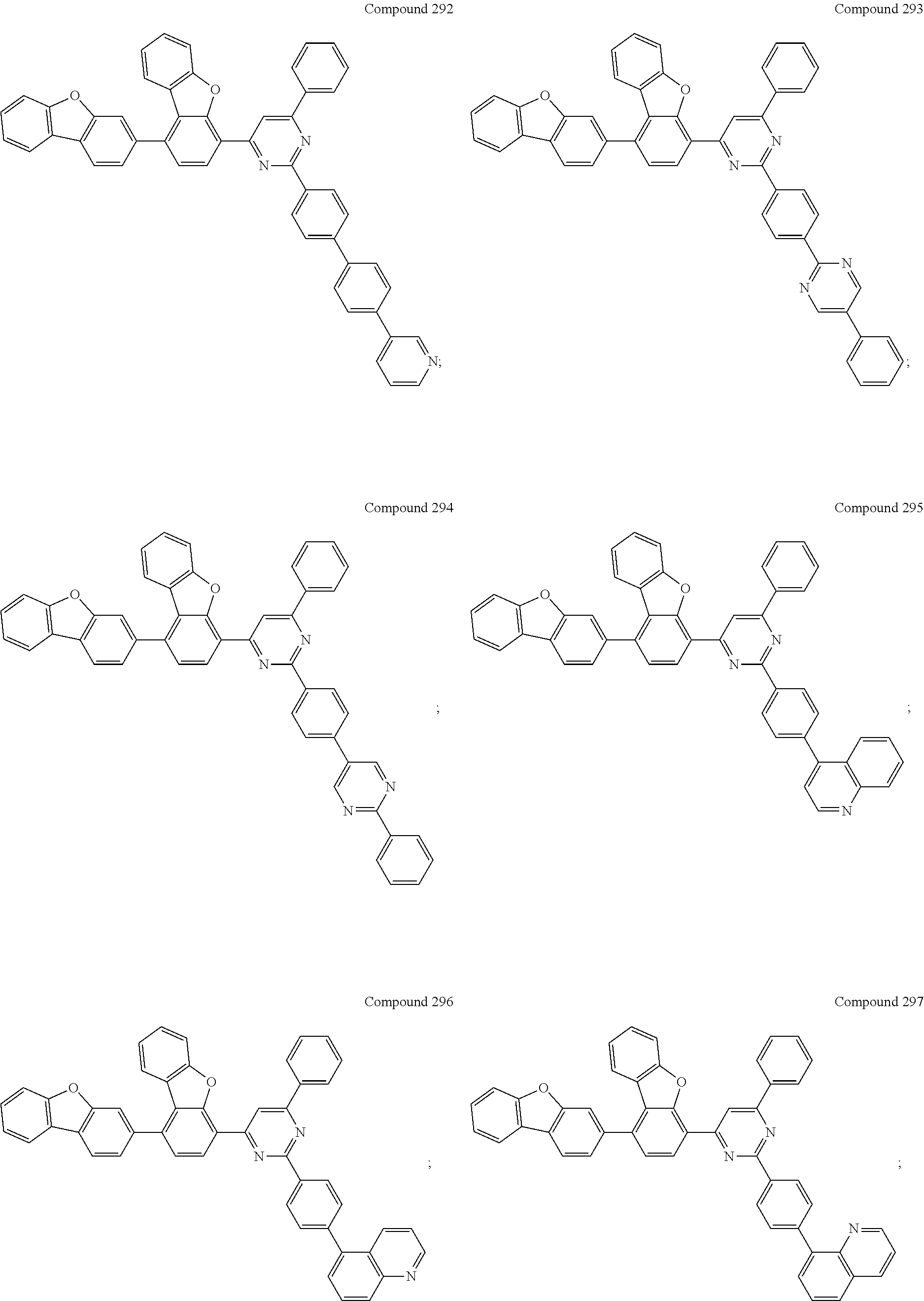

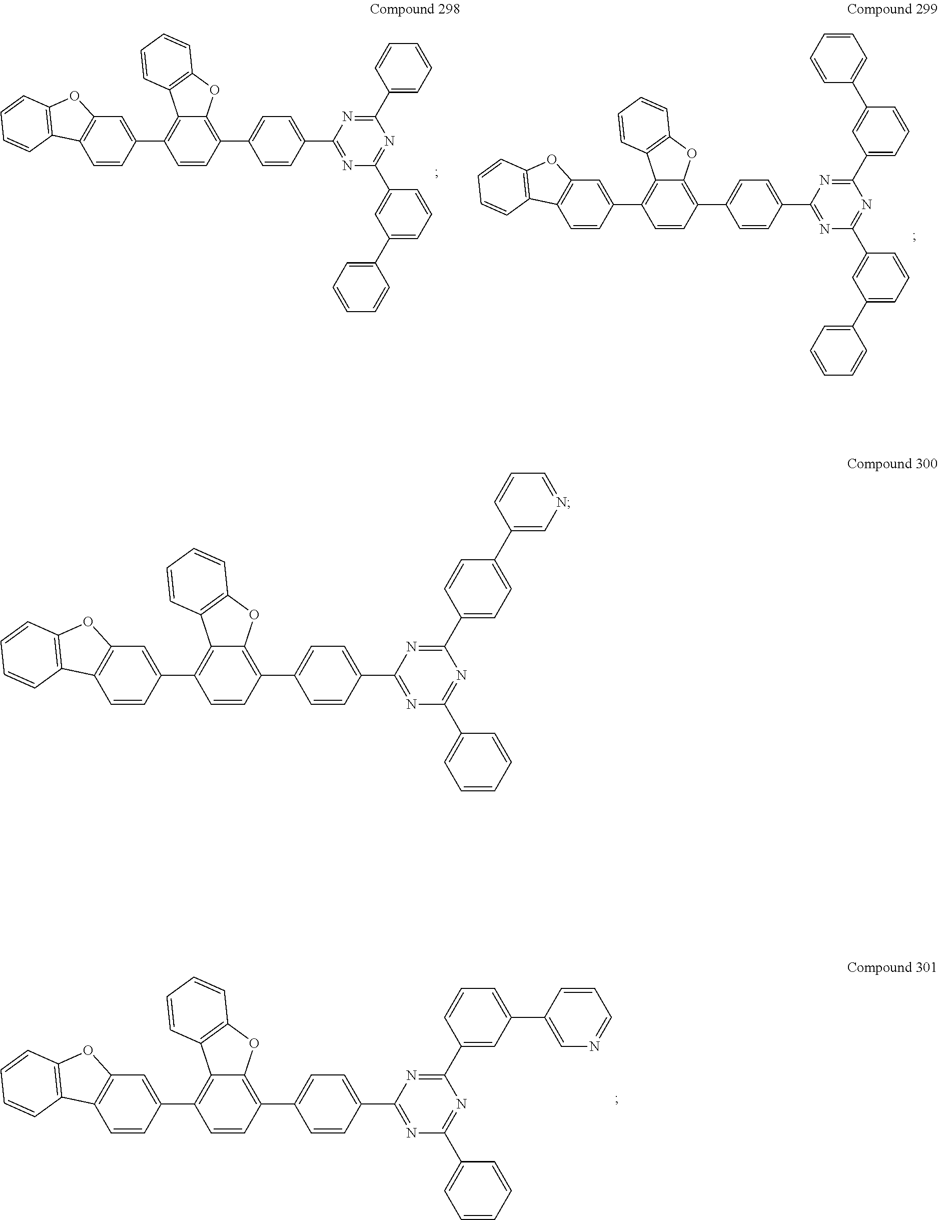 [SMILES: c1ccc(-c2cccc(-c3nc(-c4ccccc4)nc(-c4ccc(-c5ccc(-c6ccc7c(c6)oc6ccccc67)c6c5oc5ccccc56)cc4)n3)c2)cc1 ; c1ccc(-c2cccc(-c3nc(-c4ccc(-c5ccc(-c6ccc7c(c6)oc6ccccc67)c6c5oc5ccccc56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5ccc6c(c5)oc5ccccc56)c5c4oc4ccccc45)cc3)n2)cc1 ; c1ccc(-c2nc(-c3ccc(-c4ccc(-c5ccc6c(c5)oc5ccccc56)c5c4oc4ccccc45)cc3)nc(-c3cccc(-c4cccnc4)c3)n2)cc1]

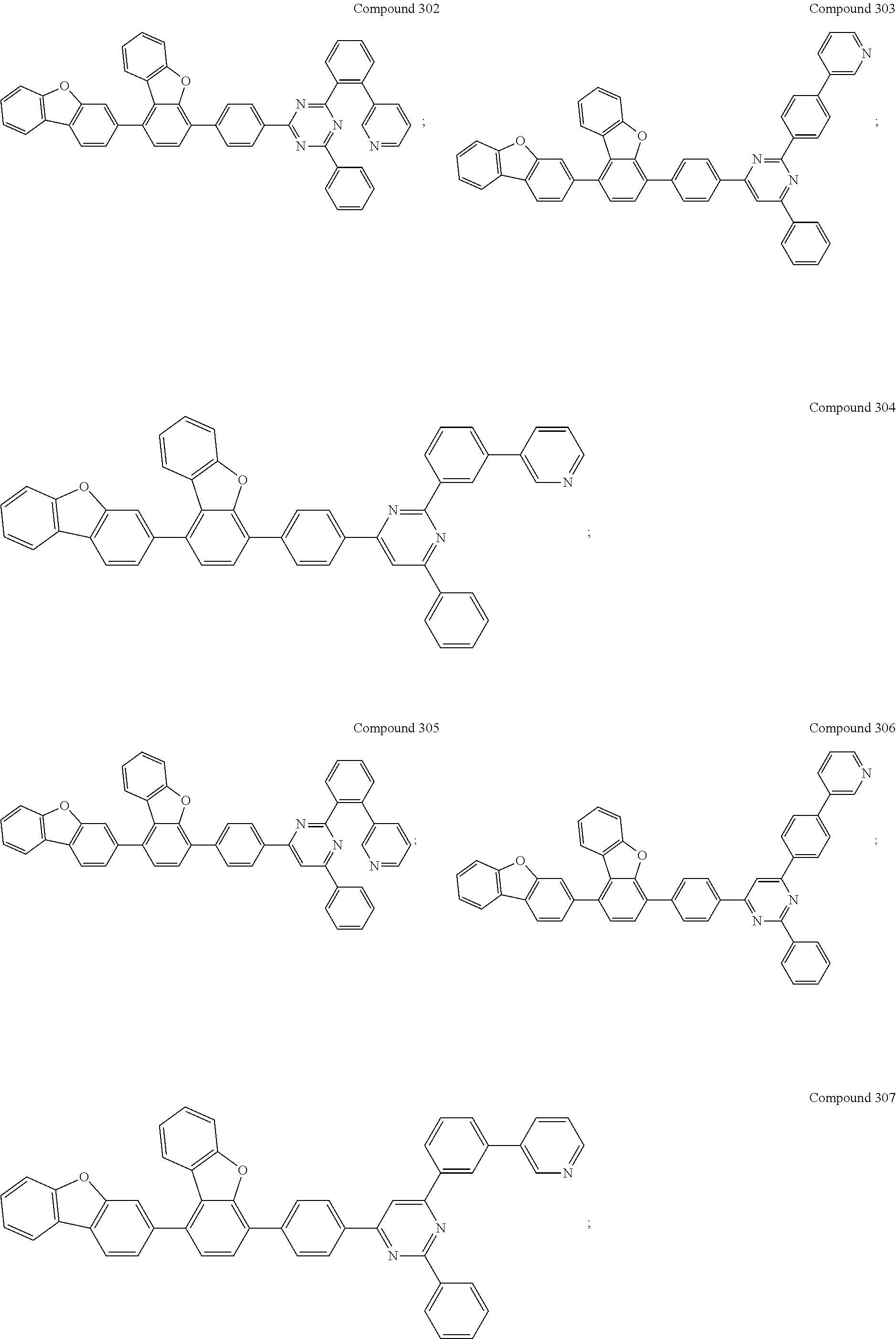

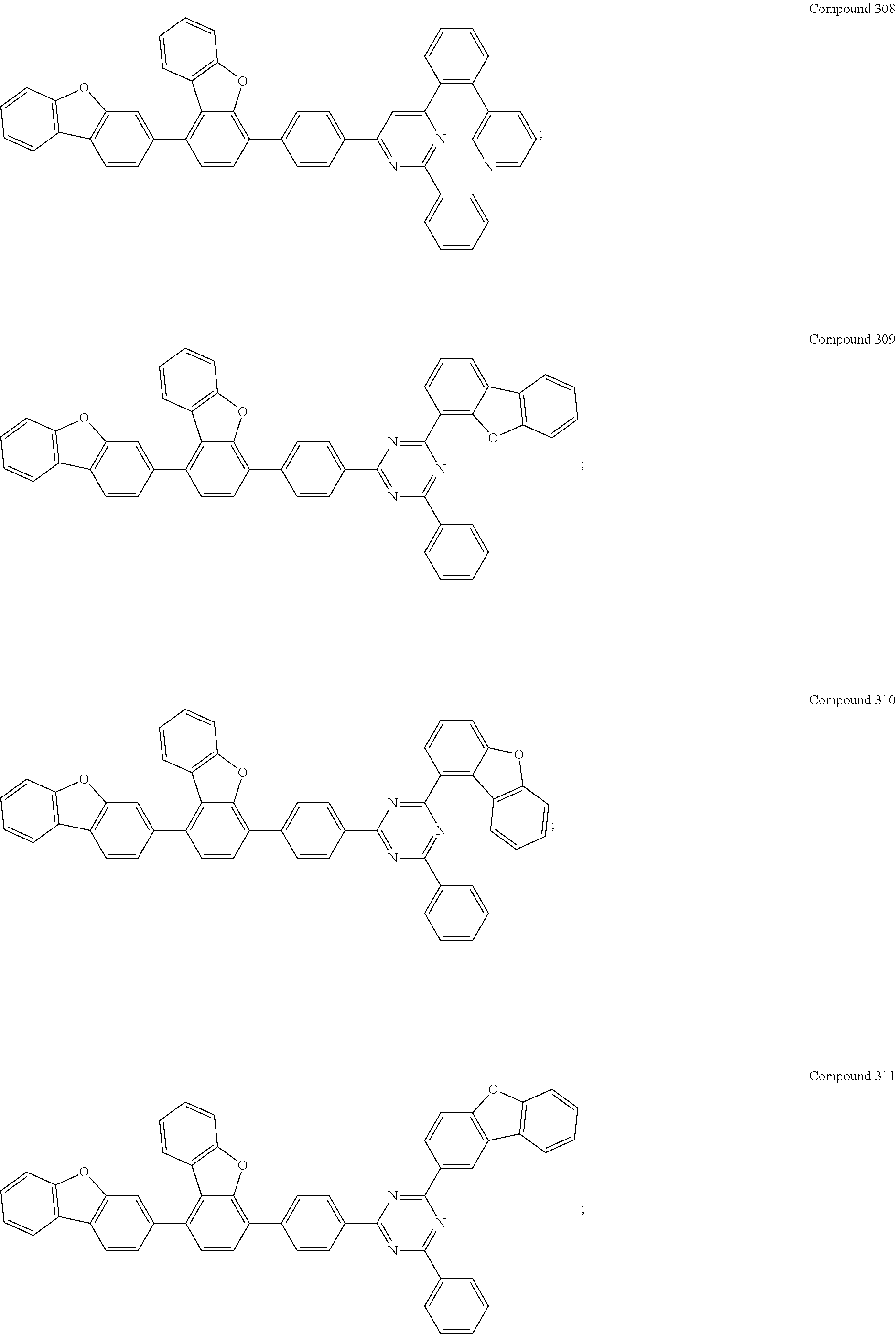 [SMILES: c1ccc(-c2nc(-c3ccc(-c4ccc(-c5ccc6c(c5)oc5ccccc56)c5c4oc4ccccc45)cc3)cc(-c3ccccc3-c3cccnc3)n2)cc1 ; c1ccc(-c2nc(-c3ccc(-c4ccc(-c5ccc6c(c5)oc5ccccc56)c5c4oc4ccccc45)cc3)nc(-c3cccc4c3oc3ccccc34)n2)cc1 ; c1ccc(-c2nc(-c3ccc(-c4ccc(-c5ccc6c(c5)oc5ccccc56)c5c4oc4ccccc45)cc3)nc(-c3cccc4oc5ccccc5c34)n2)cc1 ; c1ccc(-c2nc(-c3ccc(-c4ccc(-c5ccc6c(c5)oc5ccccc56)c5c4oc4ccccc45)cc3)nc(-c3ccc4oc5ccccc5c4c3)n2)cc1]

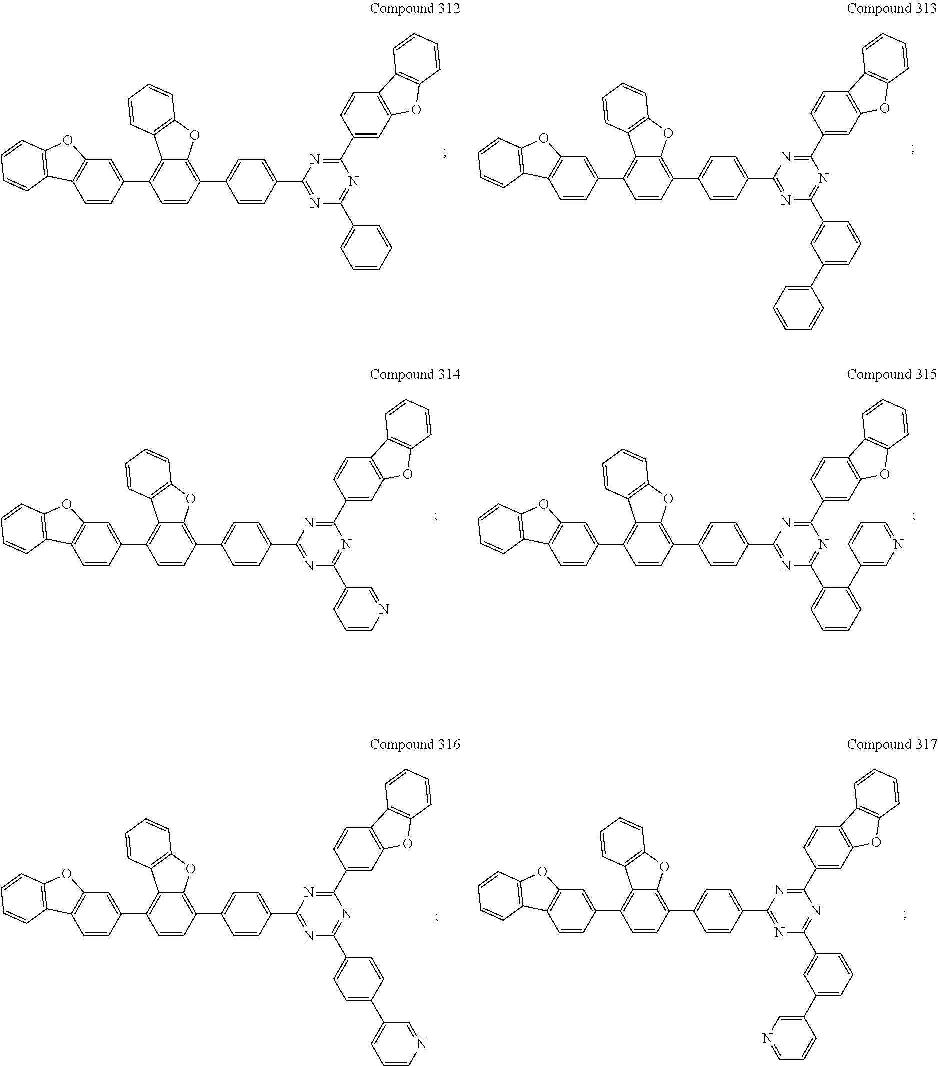

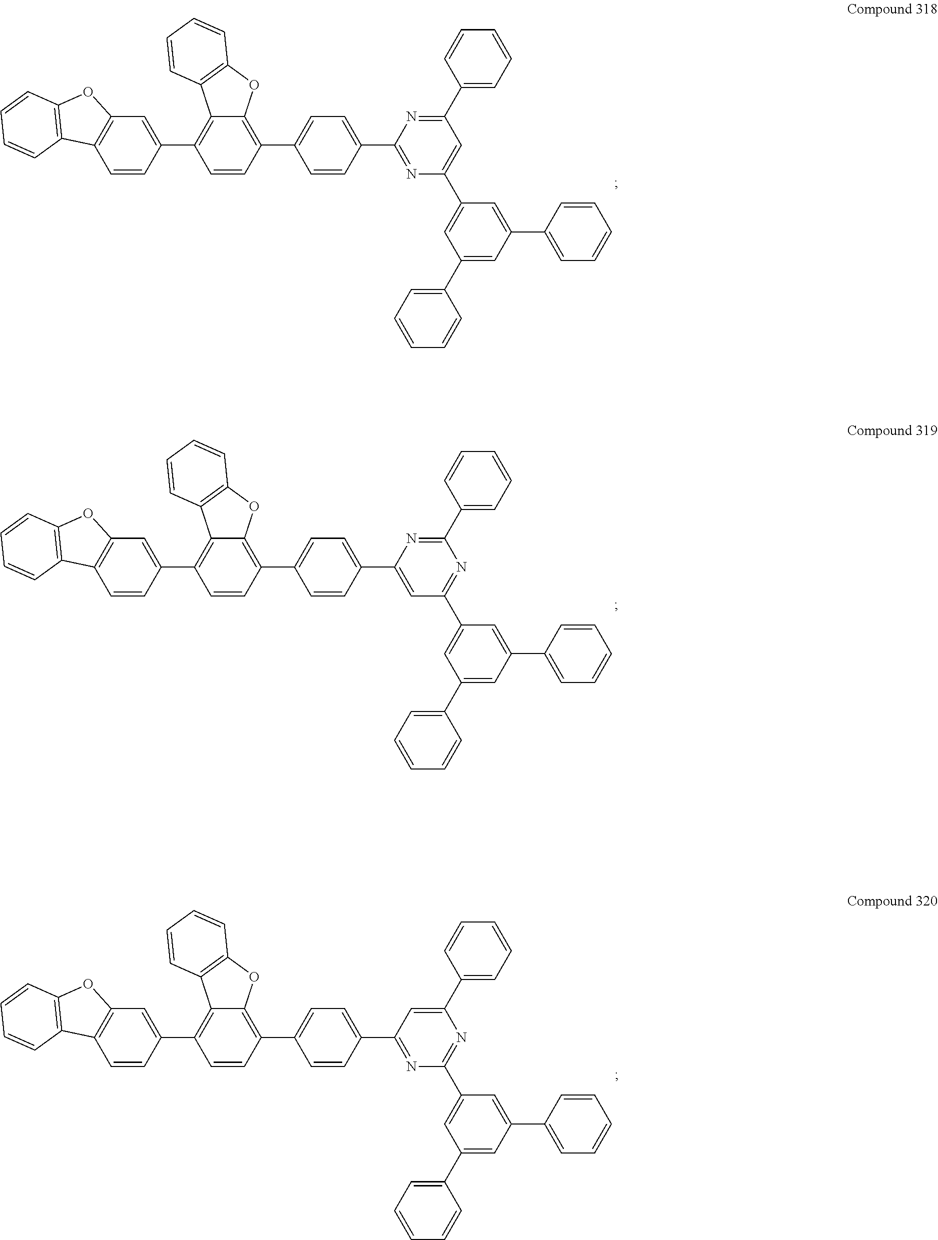 [SMILES: c1ccc(-c2cc(-c3ccccc3)cc(-c3cc(-c4ccccc4)nc(-c4ccc(-c5ccc(-c6ccc7c(c6)oc6ccccc67)c6c5oc5ccccc56)cc4)n3)c2)cc1 ; c1ccc(-c2cc(-c3ccccc3)cc(-c3cc(-c4ccc(-c5ccc(-c6ccc7c(c6)oc6ccccc67)c6c5oc5ccccc56)cc4)nc(-c4ccccc4)n3)c2)cc1 ; c1ccc(-c2cc(-c3ccccc3)cc(-c3nc(-c4ccccc4)cc(-c4ccc(-c5ccc(-c6ccc7c(c6)oc6ccccc67)c6c5oc5ccccc56)cc4)n3)c2)cc1]

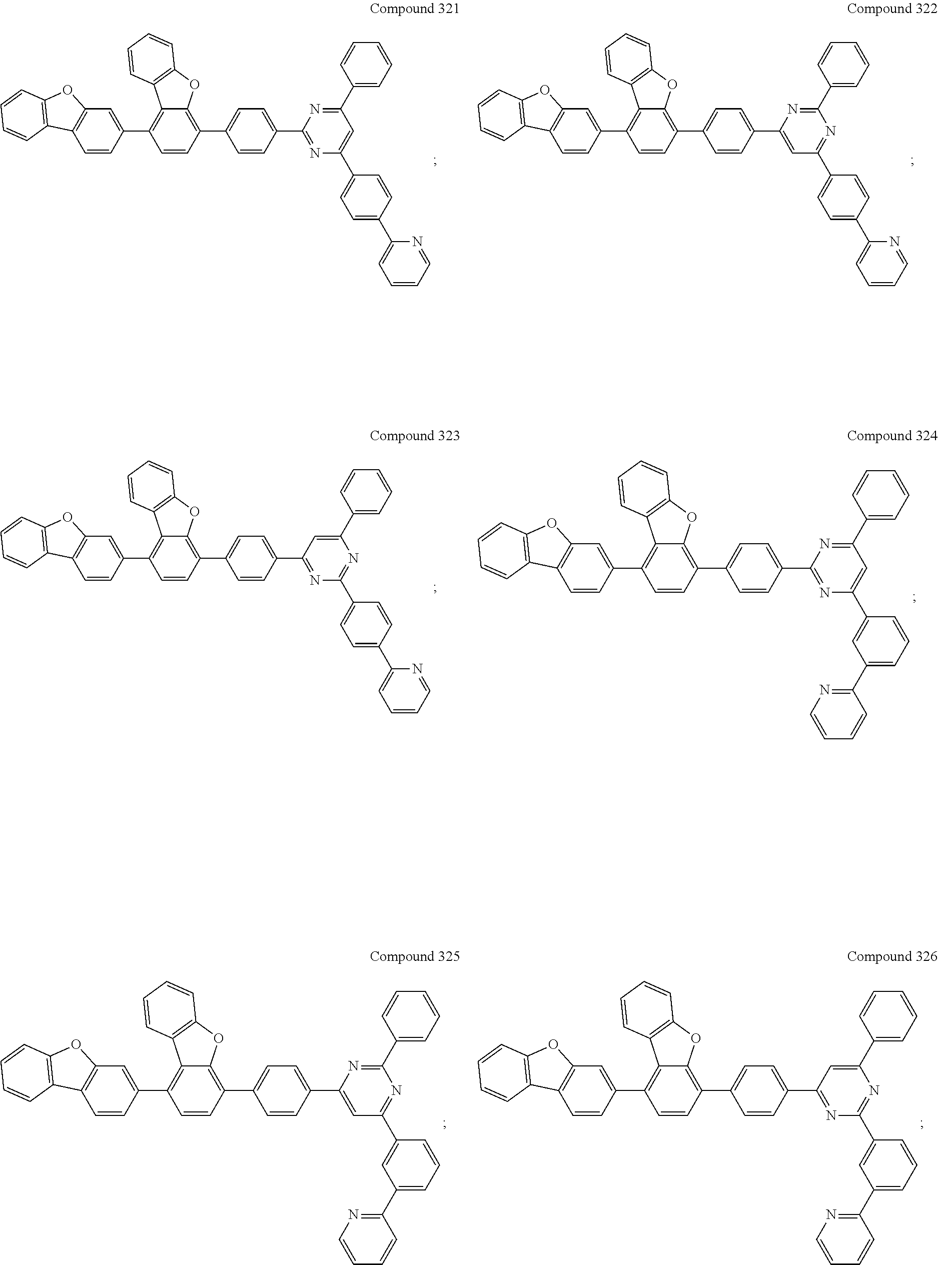

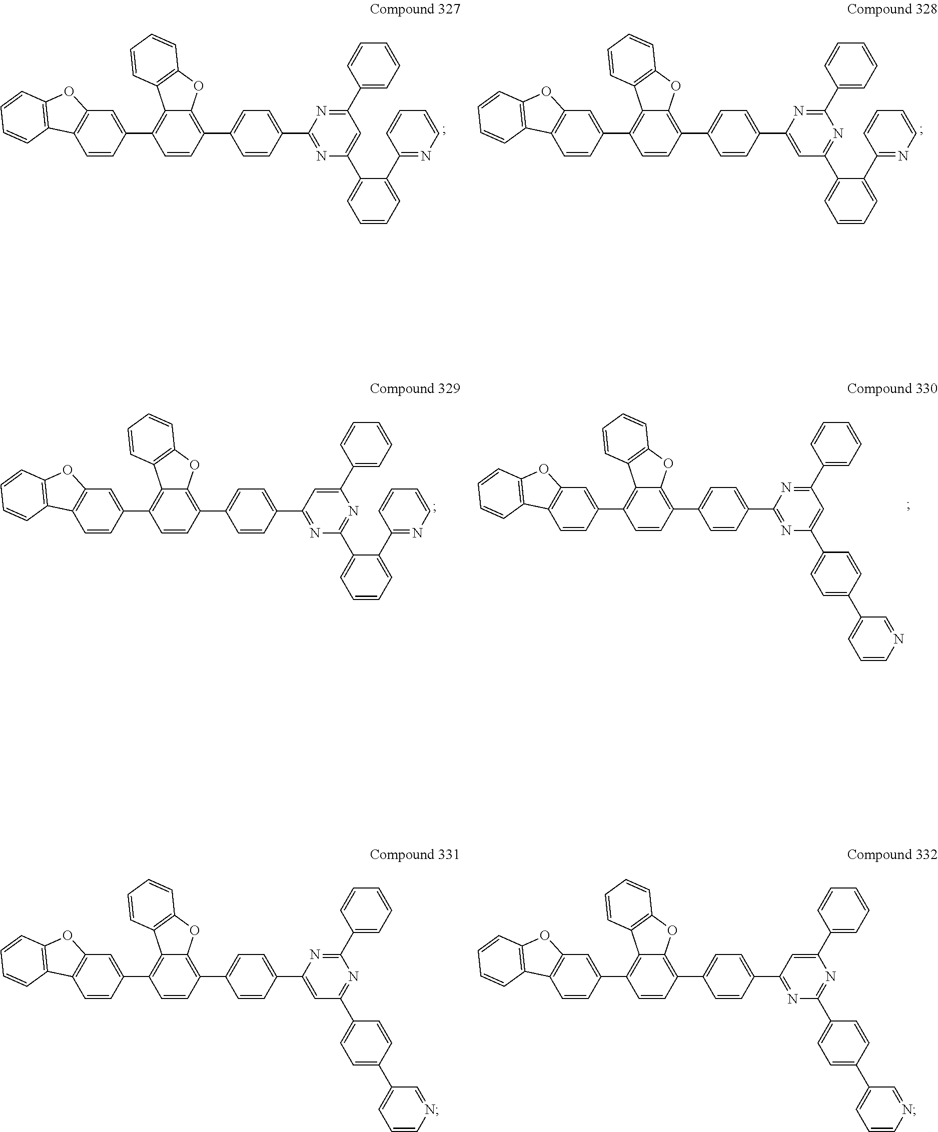

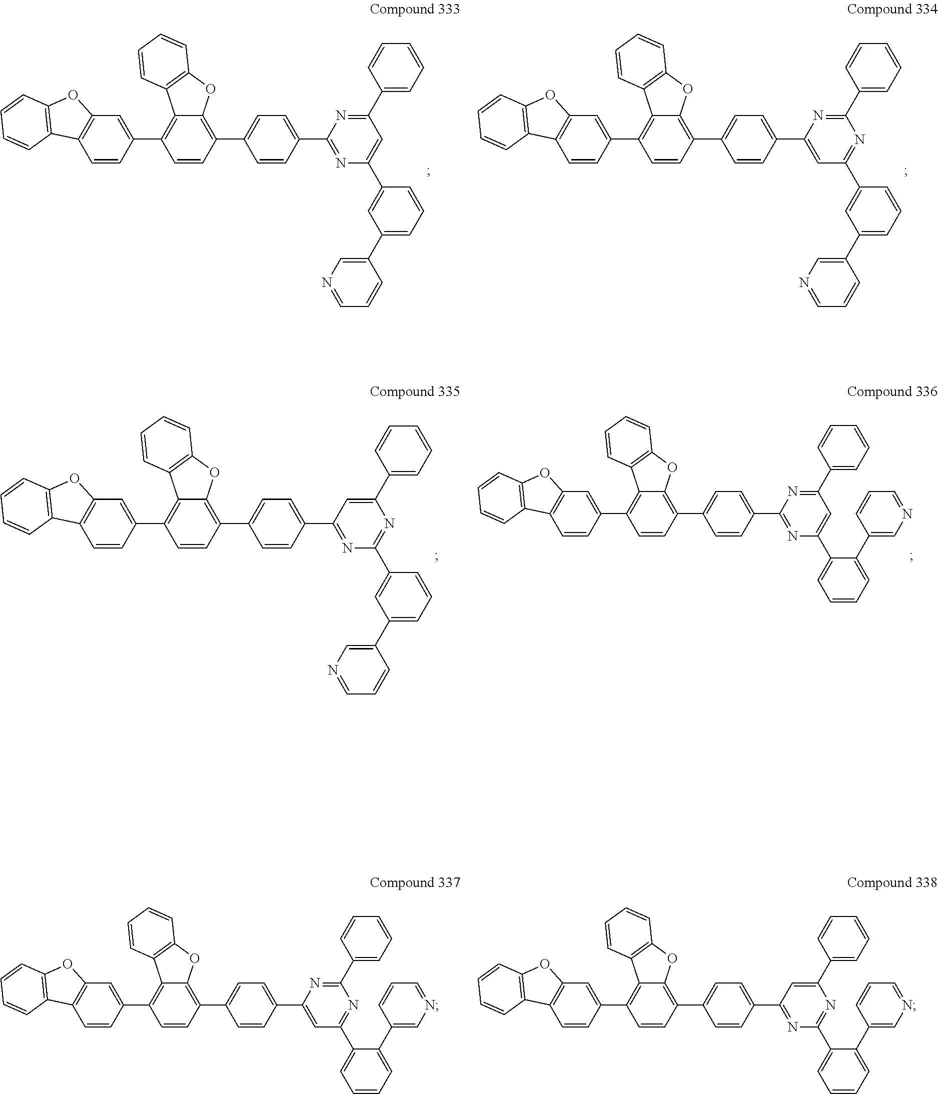

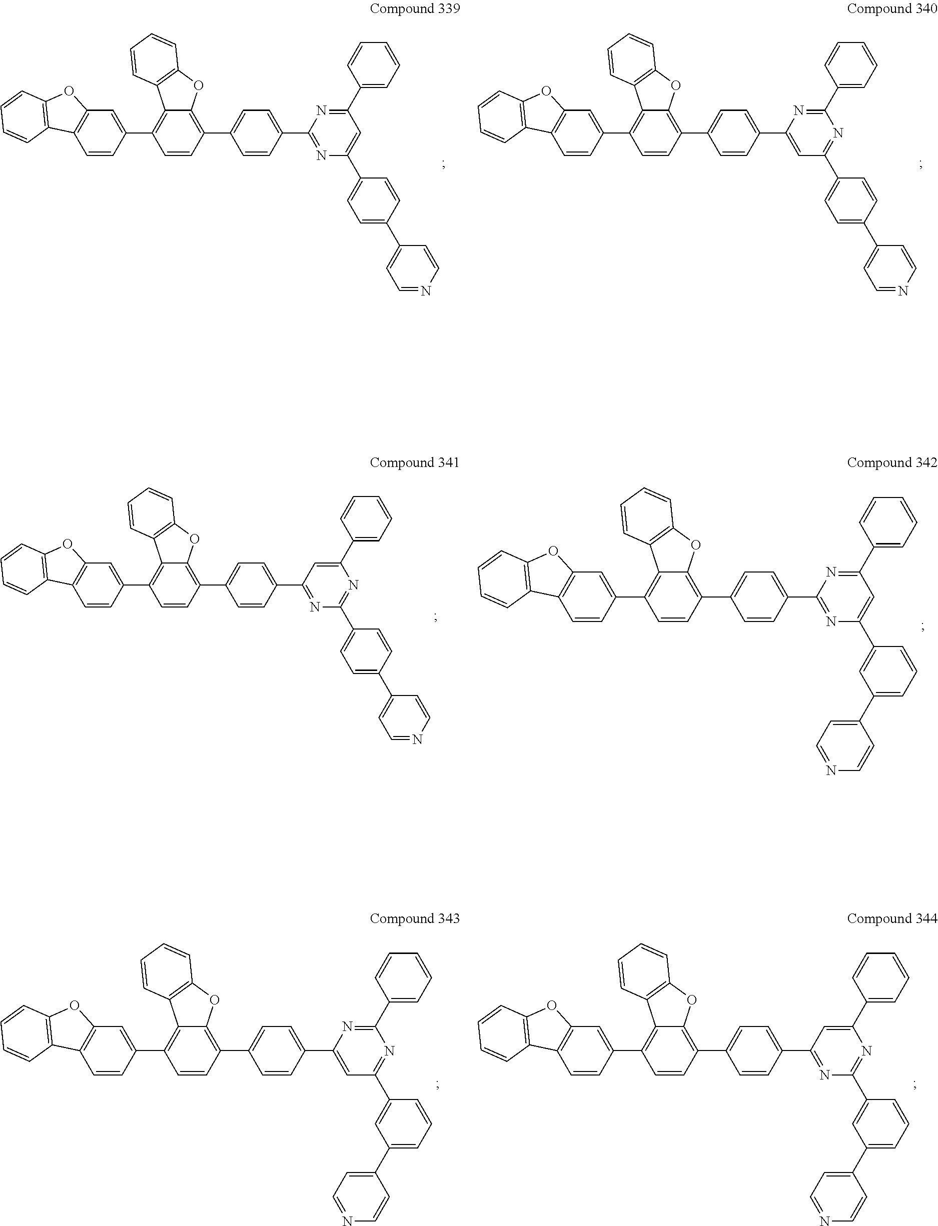

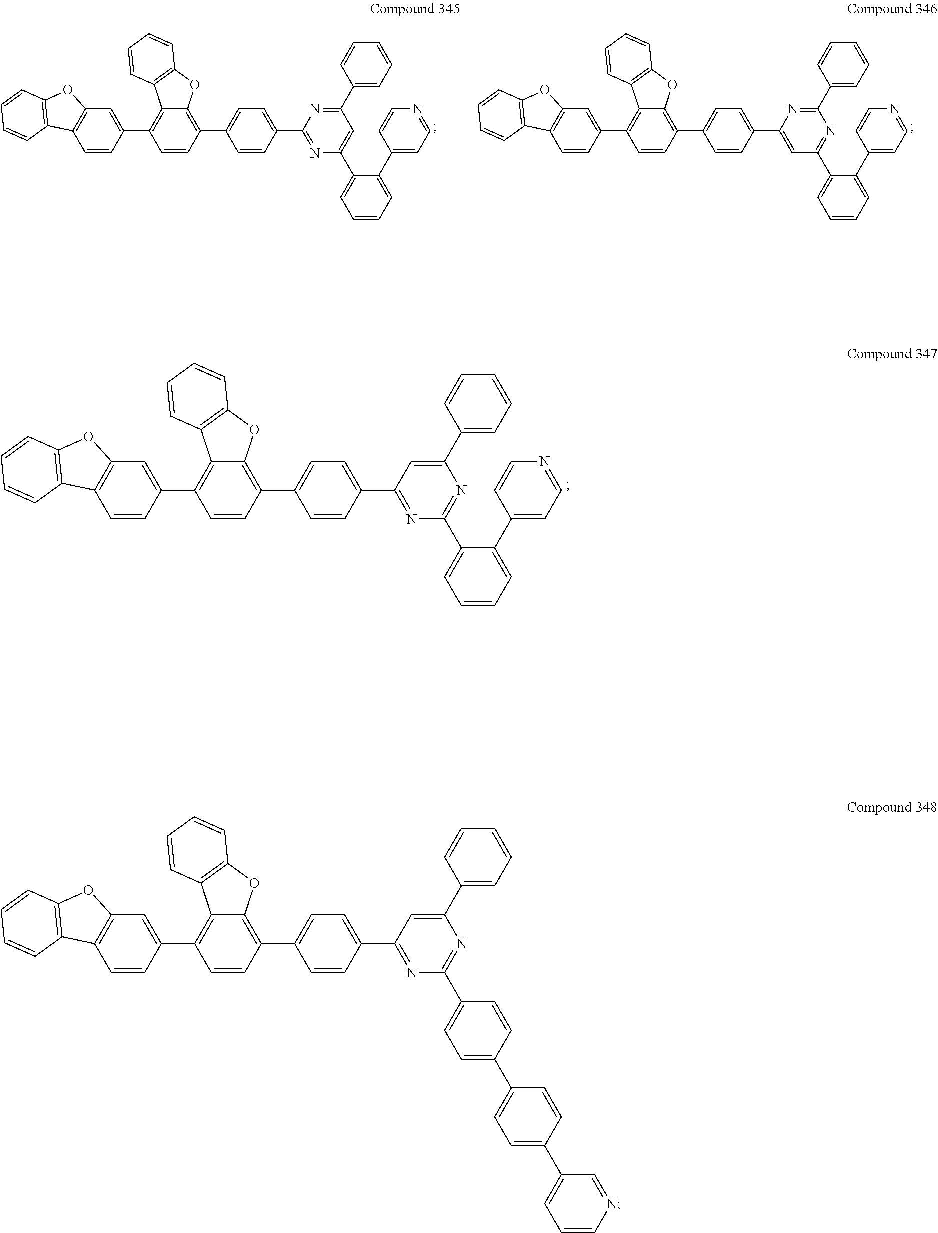 [SMILES: c1ccc(-c2cc(-c3ccccc3-c3ccncc3)nc(-c3ccc(-c4ccc(-c5ccc6c(c5)oc5ccccc56)c5c4oc4ccccc45)cc3)n2)cc1 ; c1ccc(-c2nc(-c3ccc(-c4ccc(-c5ccc6c(c5)oc5ccccc56)c5c4oc4ccccc45)cc3)cc(-c3ccccc3-c3ccncc3)n2)cc1 ; c1ccc(-c2cc(-c3ccc(-c4ccc(-c5ccc6c(c5)oc5ccccc56)c5c4oc4ccccc45)cc3)nc(-c3ccccc3-c3ccncc3)n2)cc1 ; c1ccc(-c2cc(-c3ccc(-c4ccc(-c5ccc6c(c5)oc5ccccc56)c5c4oc4ccccc45)cc3)nc(-c3ccc(-c4ccc(-c5cccnc5)cc4)cc3)n2)cc1]

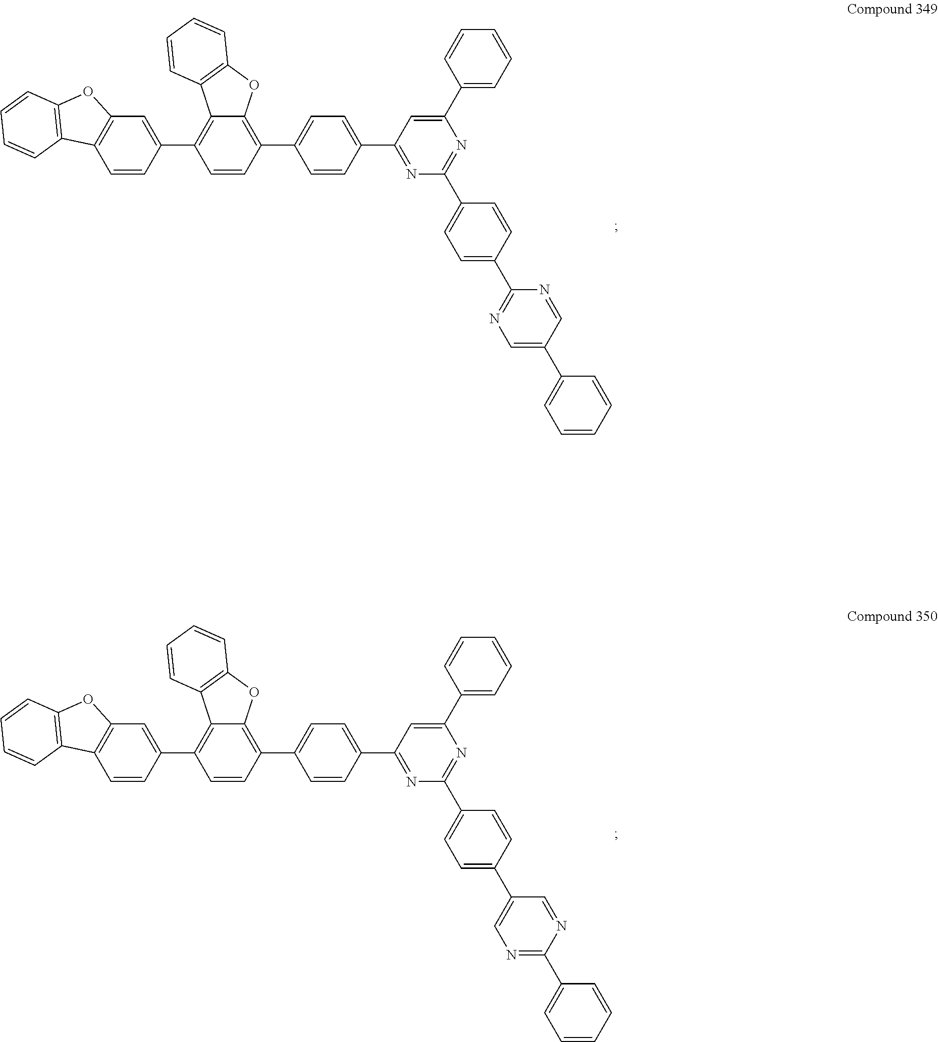 [SMILES: c1ccc(-c2cnc(-c3ccc(-c4nc(-c5ccccc5)cc(-c5ccc(-c6ccc(-c7ccc8c(c7)oc7ccccc78)c7c6oc6ccccc67)cc5)n4)cc3)nc2)cc1 ; c1ccc(-c2cc(-c3ccc(-c4ccc(-c5ccc6c(c5)oc5ccccc56)c5c4oc4ccccc45)cc3)nc(-c3ccc(-c4cnc(-c5ccccc5)nc4)cc3)n2)cc1]

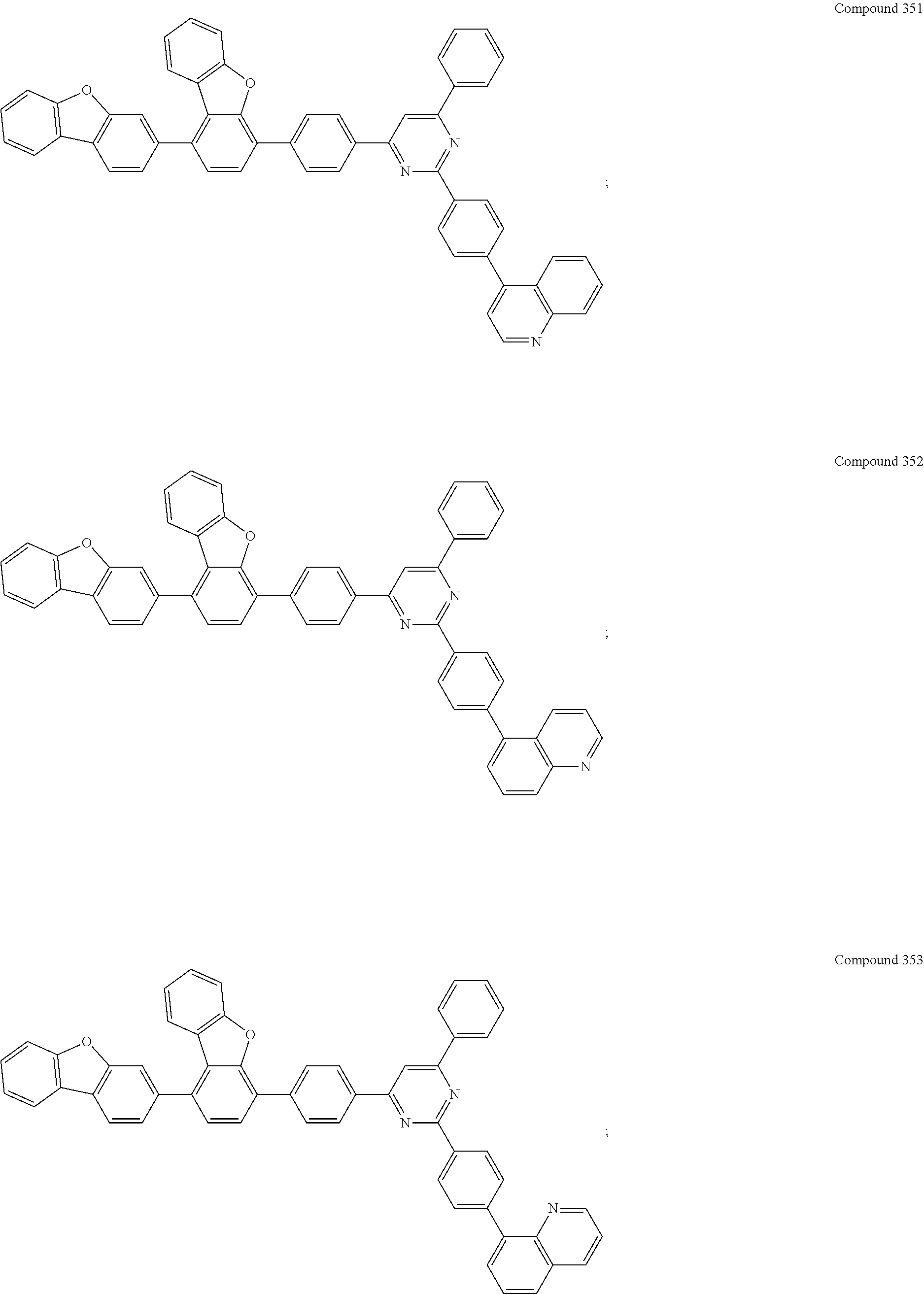 [SMILES: c1ccc(-c2cc(-c3ccc(-c4ccc(-c5ccc6c(c5)oc5ccccc56)c5c4oc4ccccc45)cc3)nc(-c3ccc(-c4ccnc5ccccc45)cc3)n2)cc1 ; c1ccc(-c2cc(-c3ccc(-c4ccc(-c5ccc6c(c5)oc5ccccc56)c5c4oc4ccccc45)cc3)nc(-c3ccc(-c4cccc5ncccc45)cc3)n2)cc1 ; c1ccc(-c2cc(-c3ccc(-c4ccc(-c5ccc6c(c5)oc5ccccc56)c5c4oc4ccccc45)cc3)nc(-c3ccc(-c4cccc5cccnc45)cc3)n2)cc1]

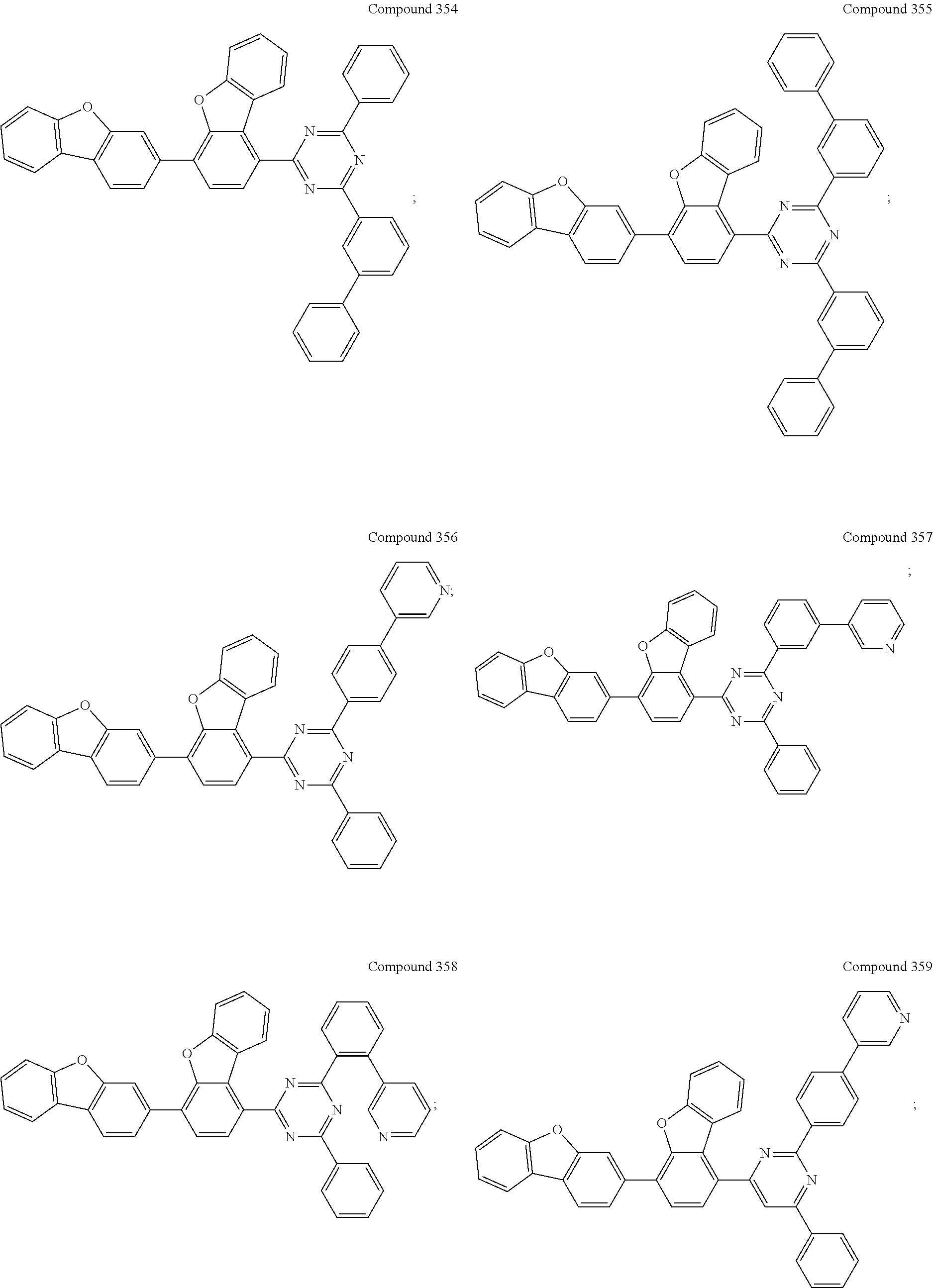

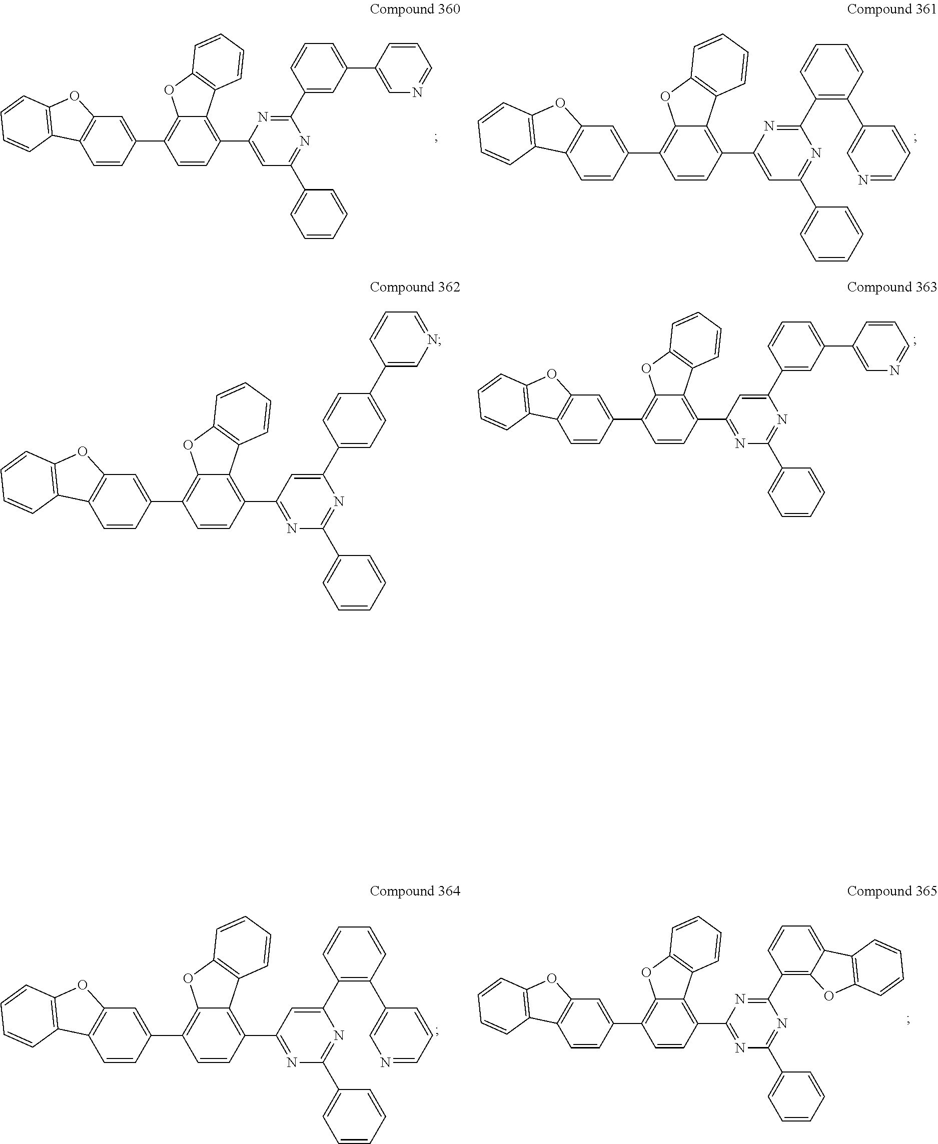

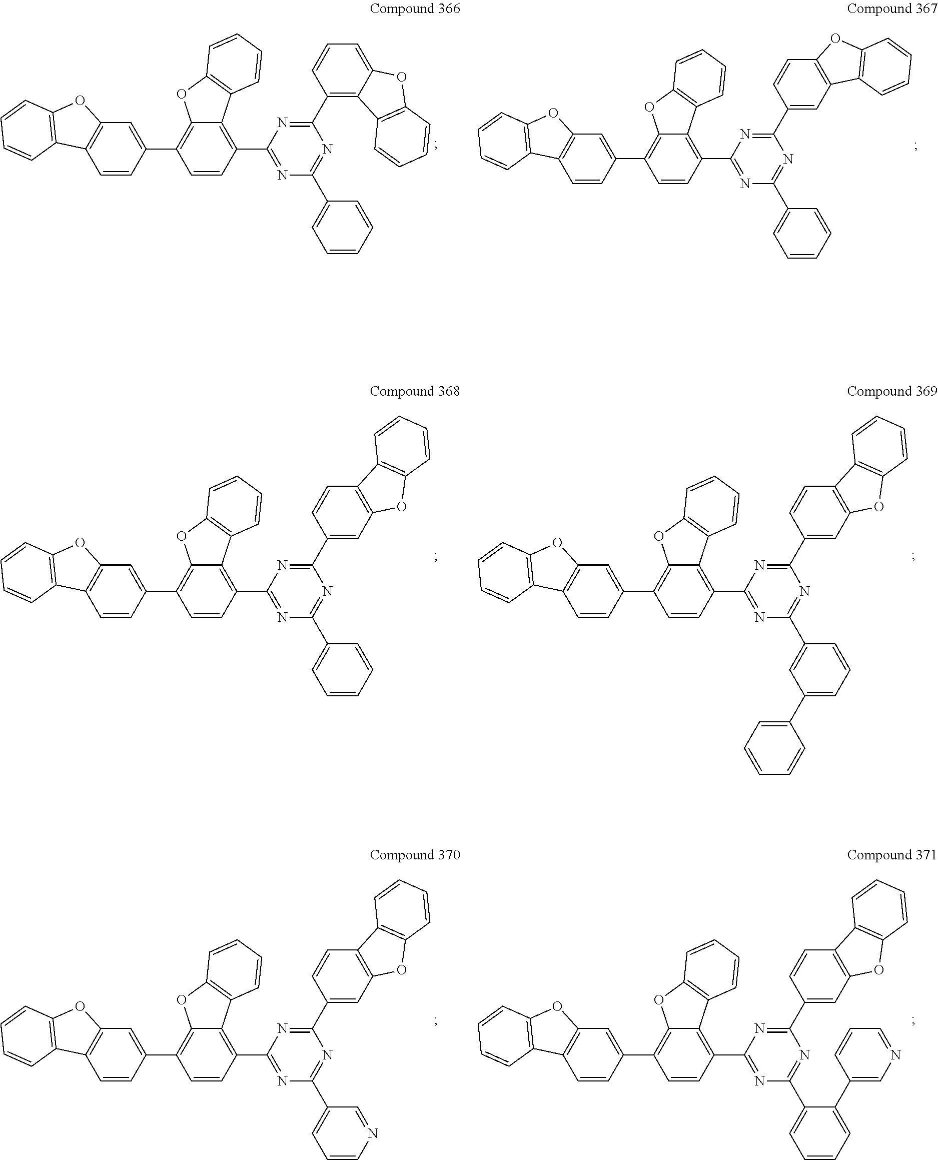

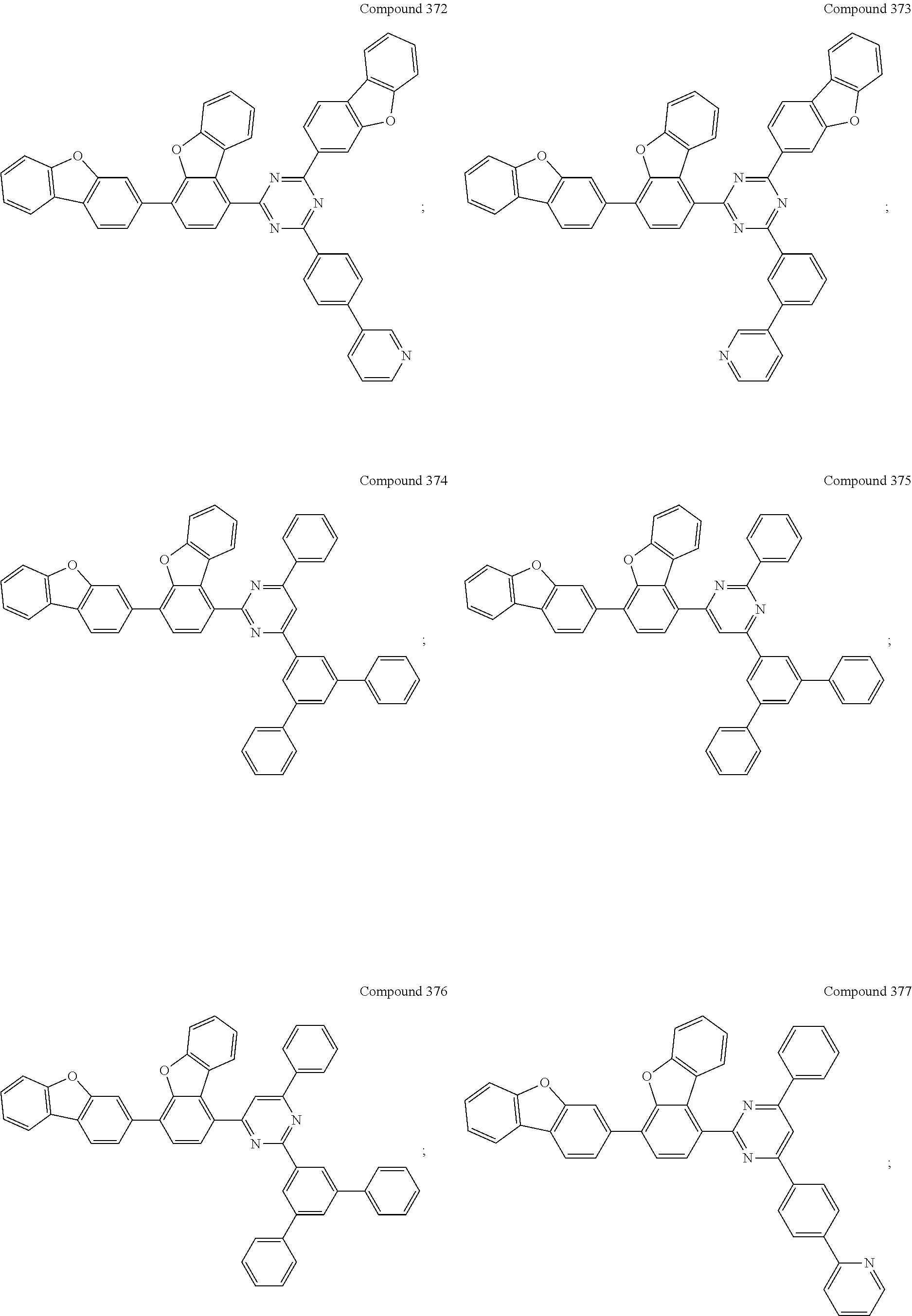

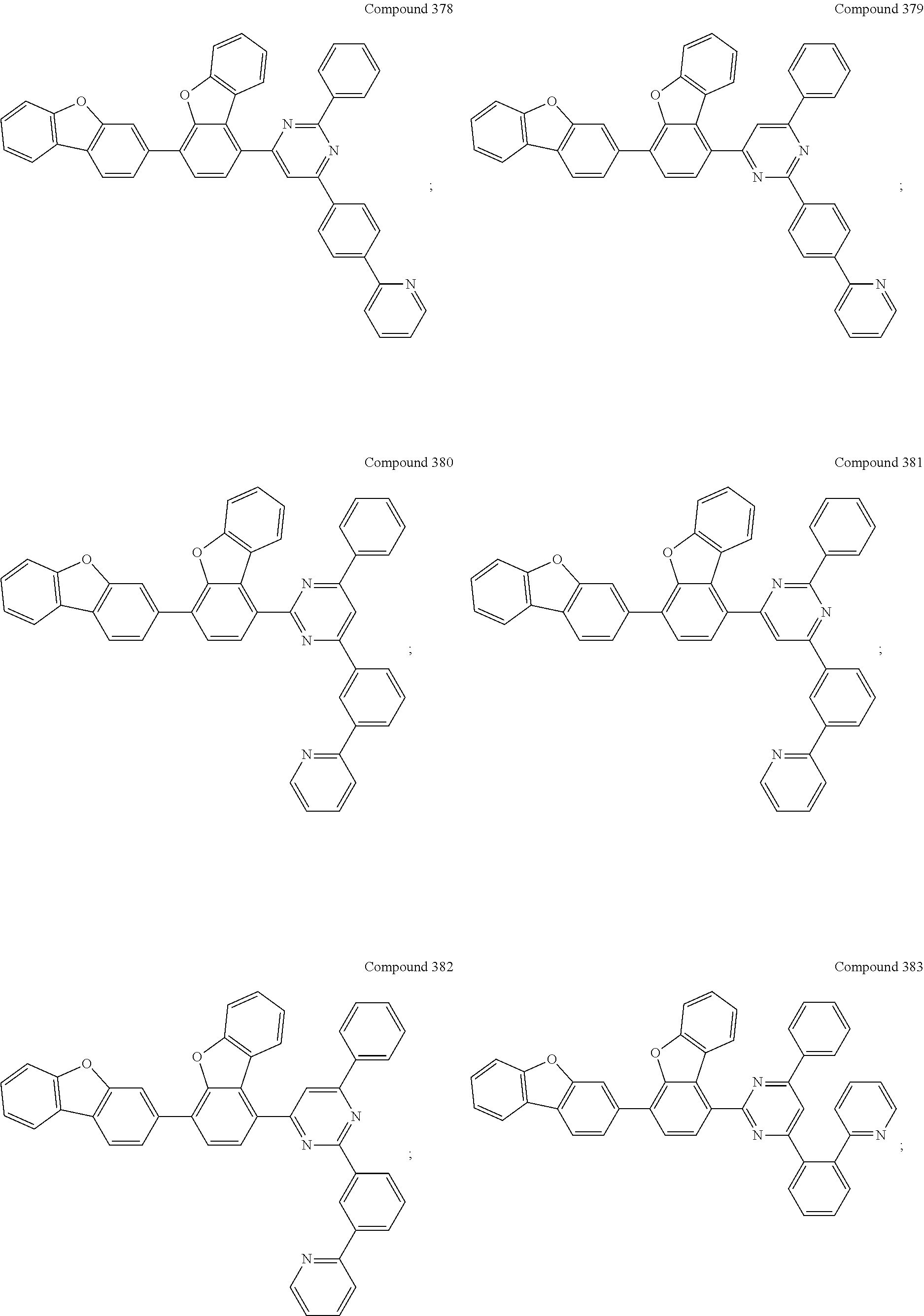

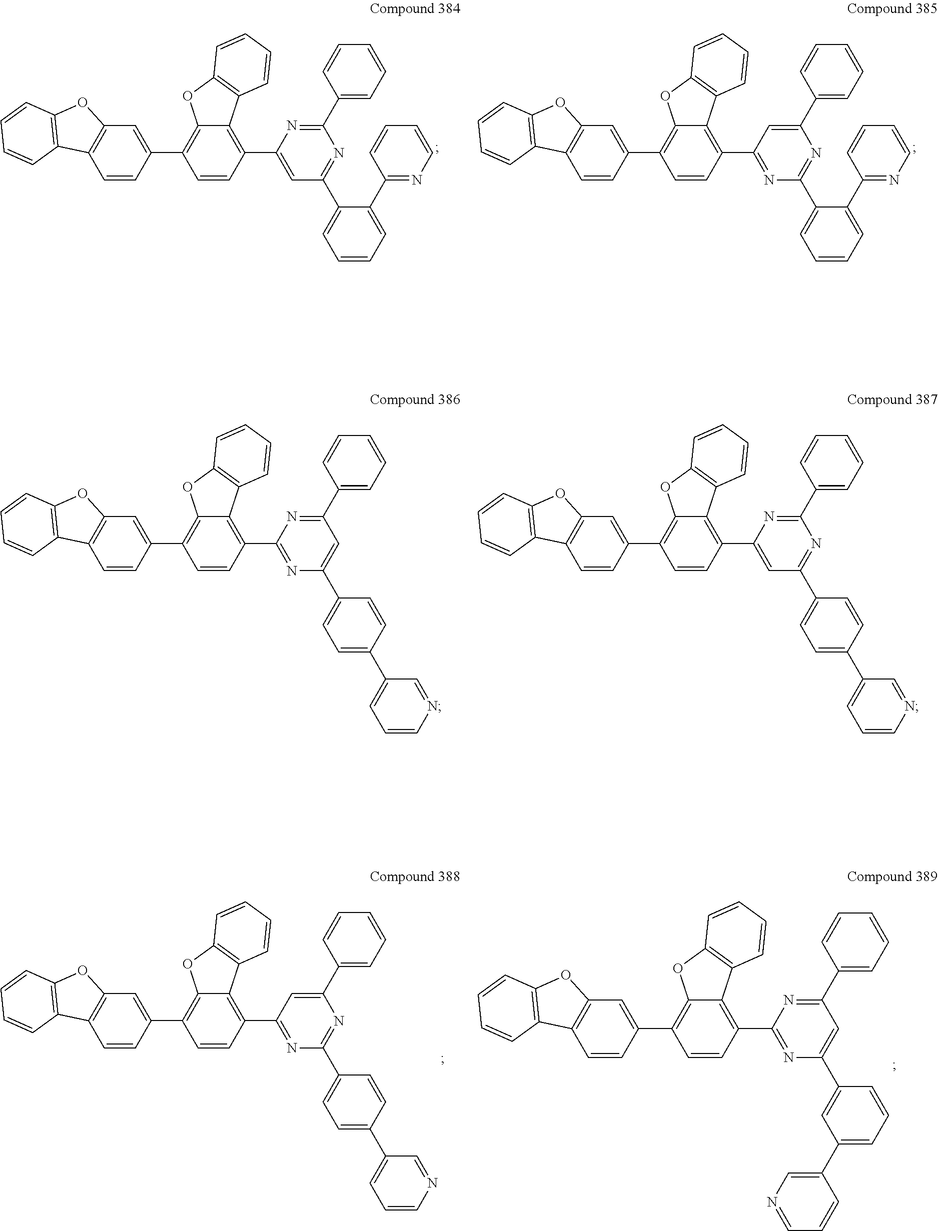

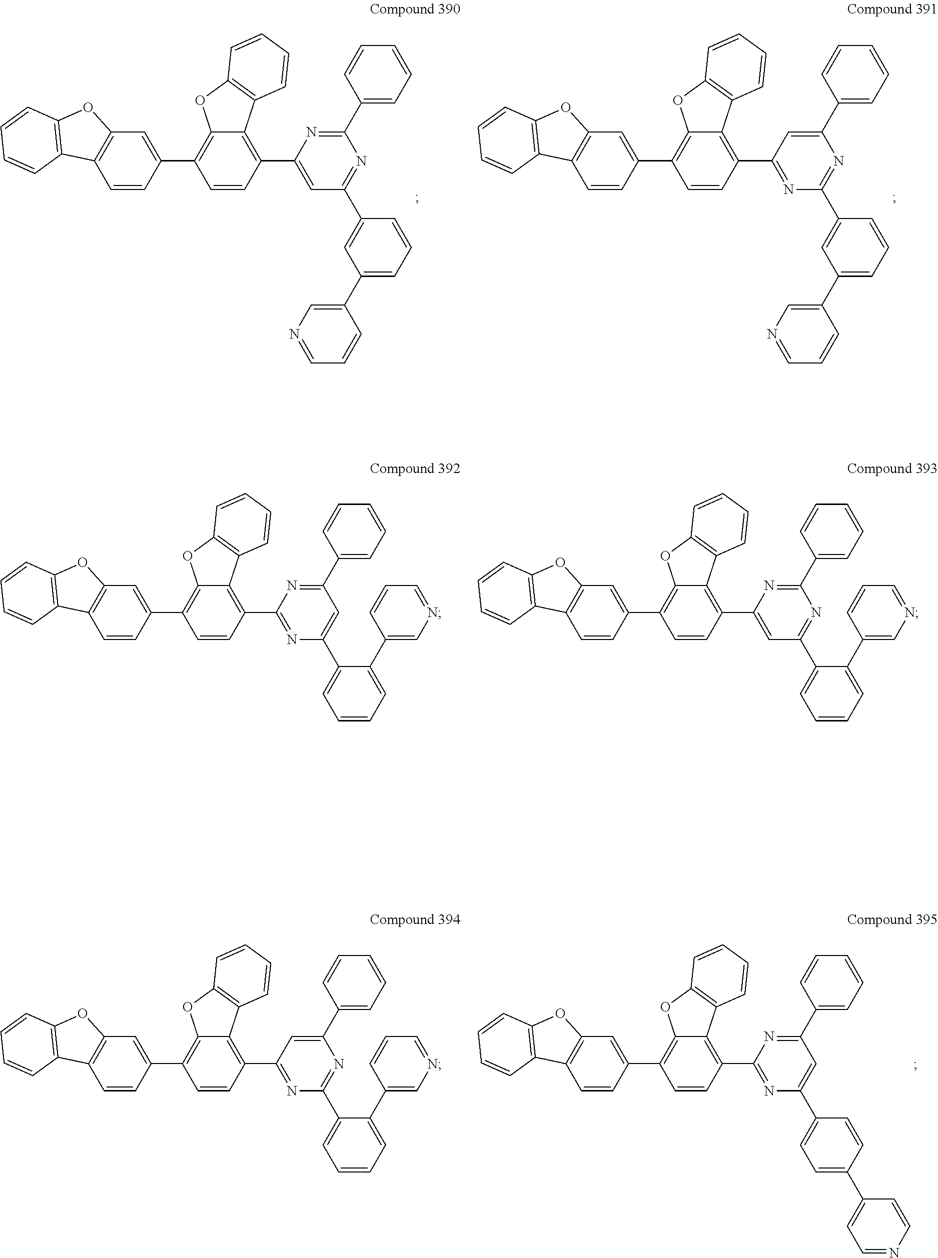

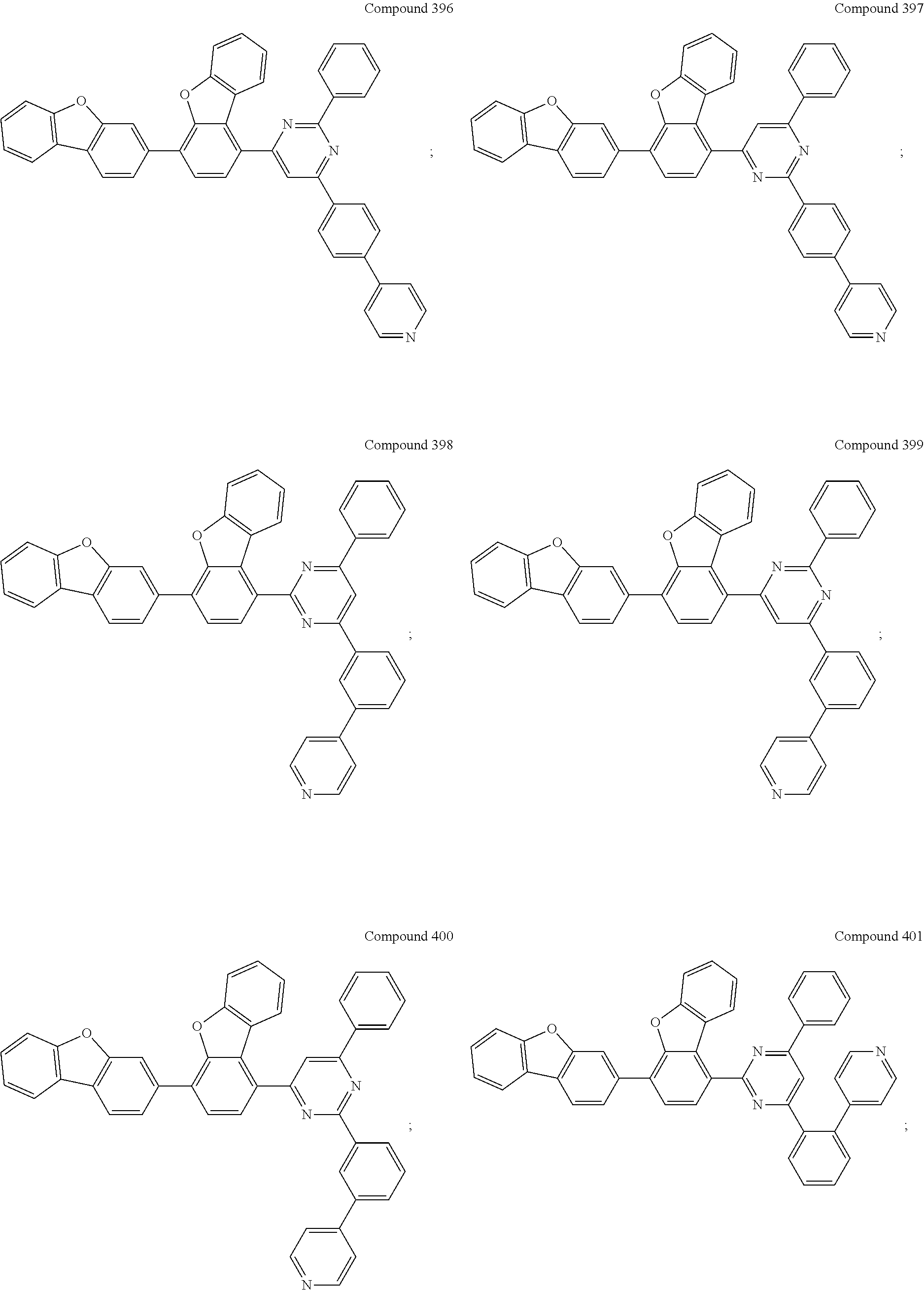

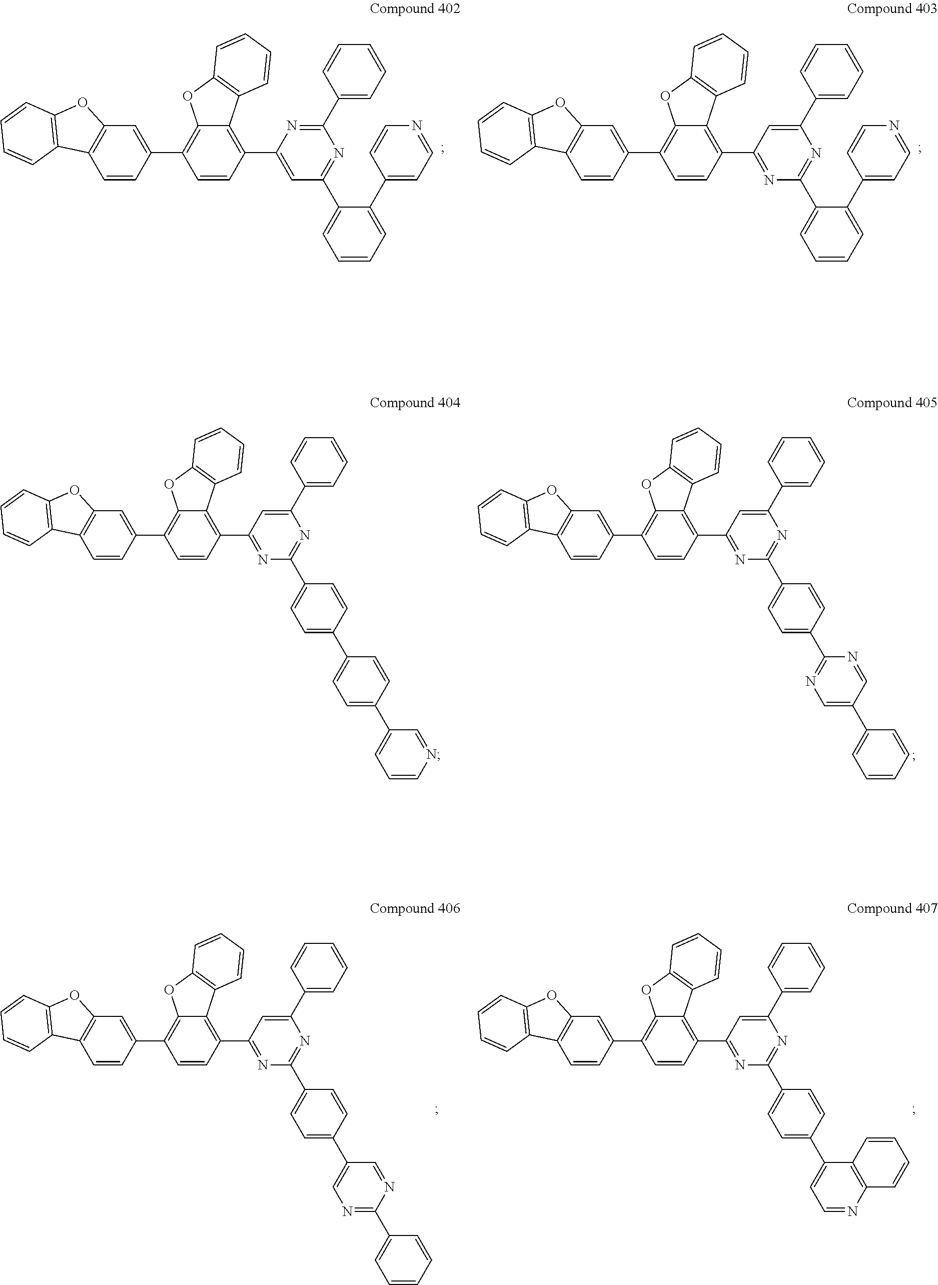

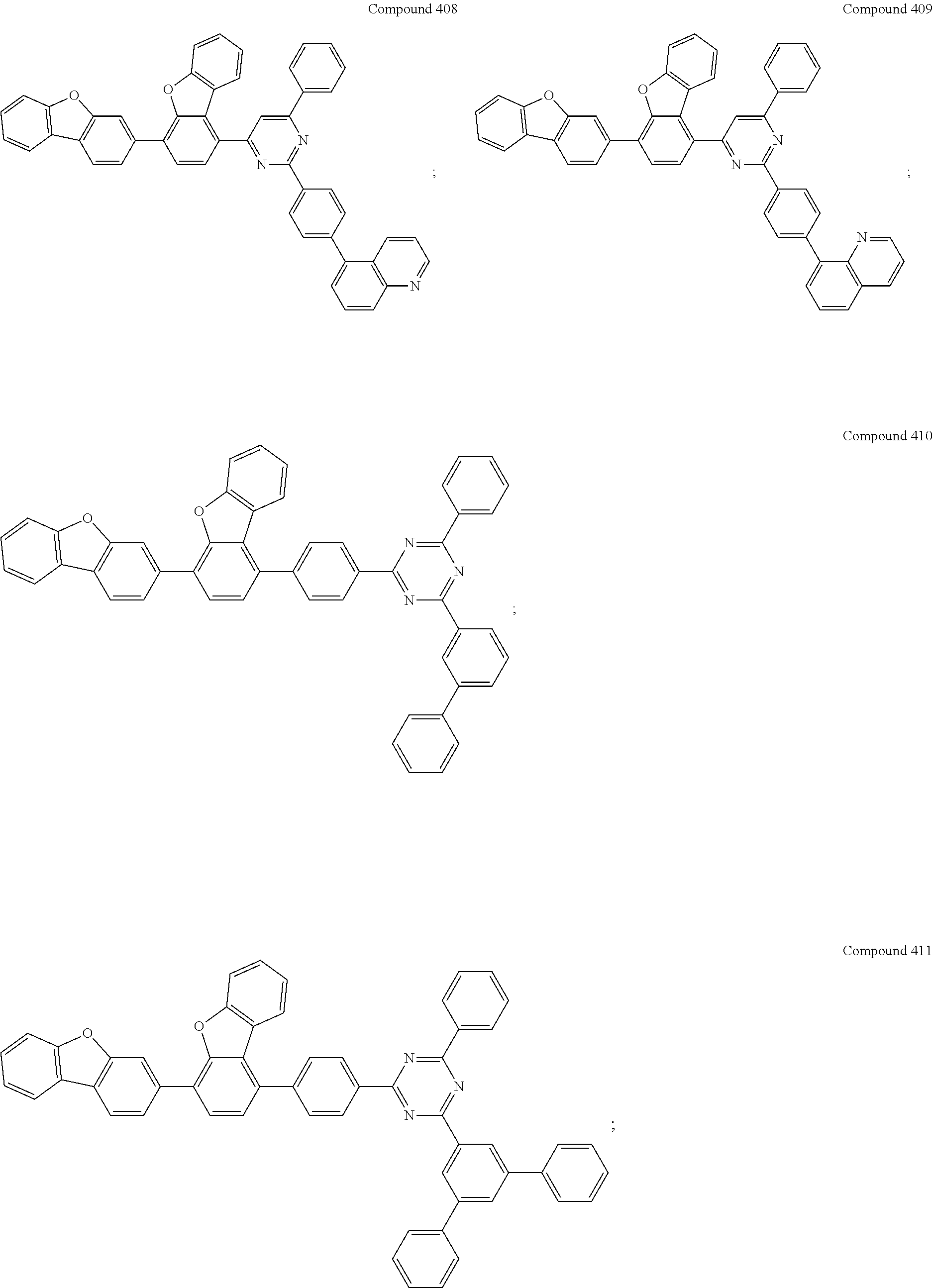 [SMILES: c1ccc(-c2cc(-c3ccc(-c4ccc5c(c4)oc4ccccc45)c4oc5ccccc5c34)nc(-c3ccc(-c4cccc5ncccc45)cc3)n2)cc1 ; c1ccc(-c2cc(-c3ccc(-c4ccc5c(c4)oc4ccccc45)c4oc5ccccc5c34)nc(-c3ccc(-c4cccc5cccnc45)cc3)n2)cc1 ; c1ccc(-c2cccc(-c3nc(-c4ccccc4)nc(-c4ccc(-c5ccc(-c6ccc7c(c6)oc6ccccc67)c6oc7ccccc7c56)cc4)n3)c2)cc1 ; c1ccc(-c2cc(-c3ccccc3)cc(-c3nc(-c4ccccc4)nc(-c4ccc(-c5ccc(-c6ccc7c(c6)oc6ccccc67)c6oc7ccccc7c56)cc4)n3)c2)cc1]

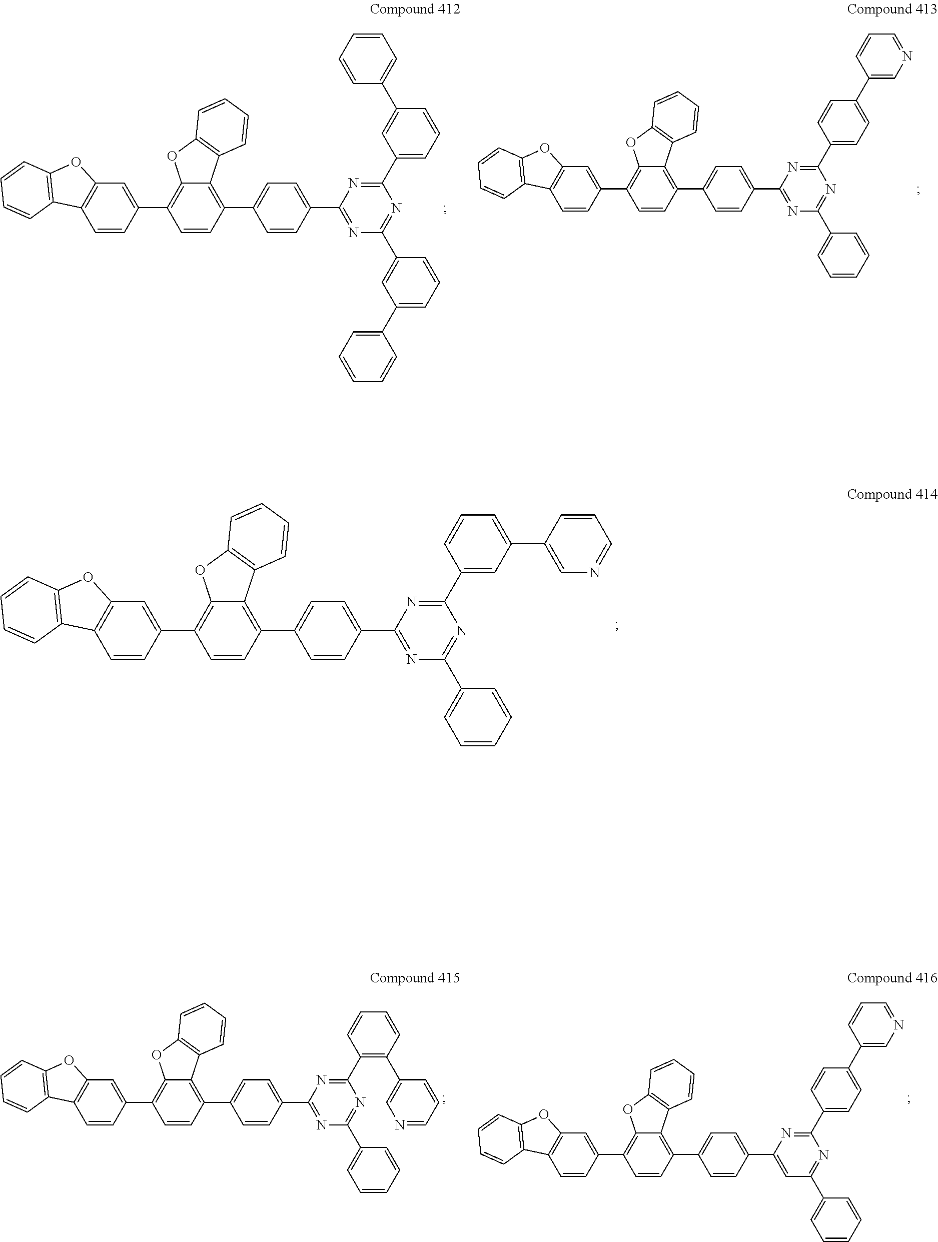

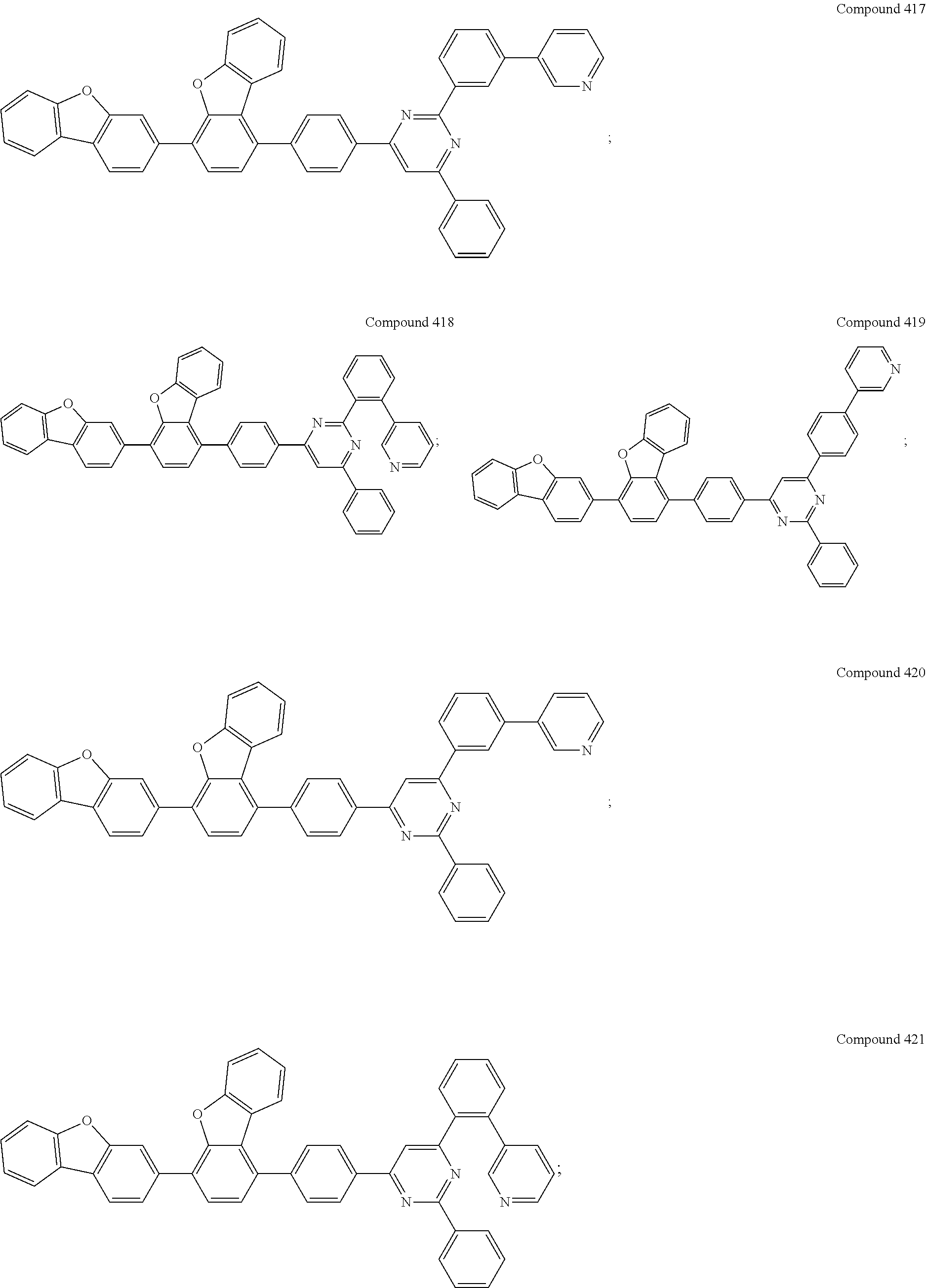 [SMILES: c1ccc(-c2cc(-c3ccc(-c4ccc(-c5ccc6c(c5)oc5ccccc56)c5oc6ccccc6c45)cc3)nc(-c3cccc(-c4cccnc4)c3)n2)cc1 ; c1ccc(-c2cc(-c3ccc(-c4ccc(-c5ccc6c(c5)oc5ccccc56)c5oc6ccccc6c45)cc3)nc(-c3ccccc3-c3cccnc3)n2)cc1 ; c1ccc(-c2nc(-c3ccc(-c4cccnc4)cc3)cc(-c3ccc(-c4ccc(-c5ccc6c(c5)oc5ccccc56)c5oc6ccccc6c45)cc3)n2)cc1 ; c1ccc(-c2nc(-c3ccc(-c4ccc(-c5ccc6c(c5)oc5ccccc56)c5oc6ccccc6c45)cc3)cc(-c3cccc(-c4cccnc4)c3)n2)cc1 ; c1ccc(-c2nc(-c3ccc(-c4ccc(-c5ccc6c(c5)oc5ccccc56)c5oc6ccccc6c45)cc3)cc(-c3ccccc3-c3cccnc3)n2)cc1]

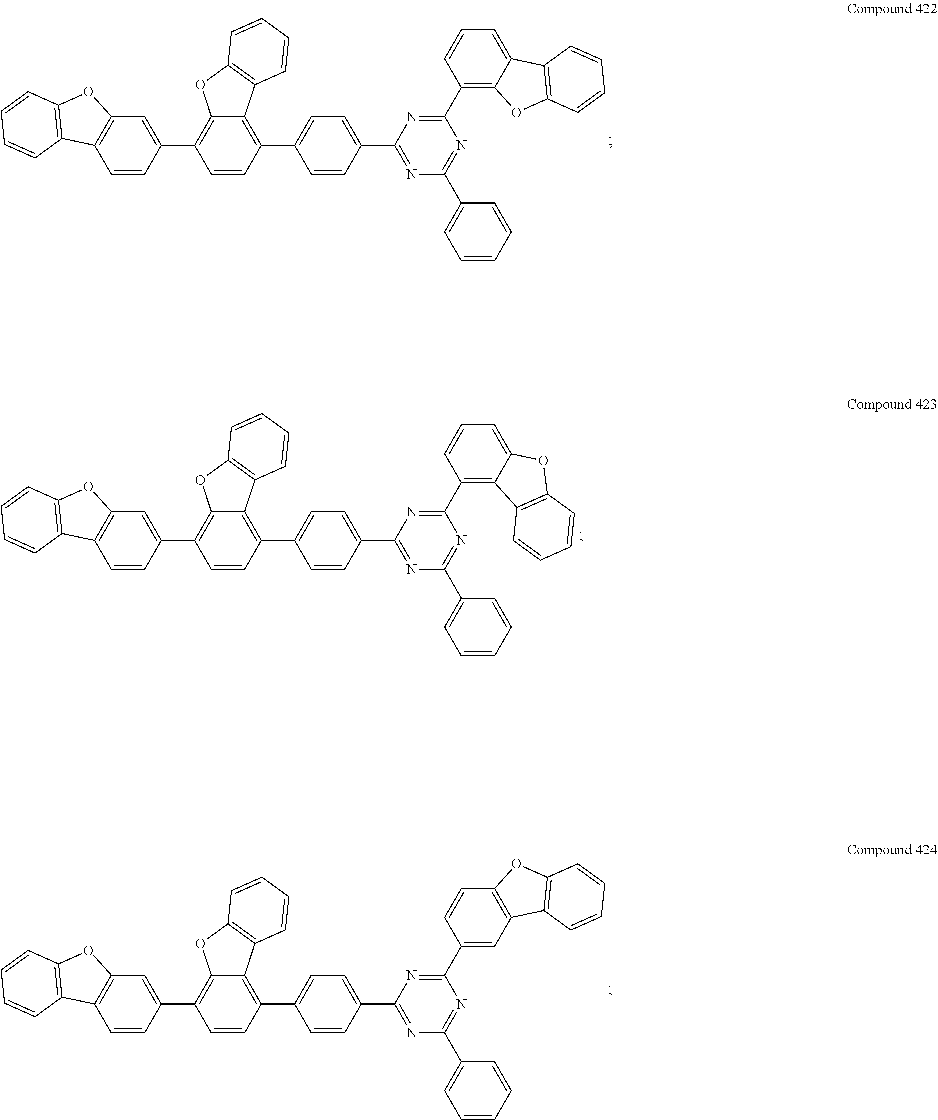 [SMILES: c1ccc(-c2nc(-c3ccc(-c4ccc(-c5ccc6c(c5)oc5ccccc56)c5oc6ccccc6c45)cc3)nc(-c3cccc4c3oc3ccccc34)n2)cc1 ; c1ccc(-c2nc(-c3ccc(-c4ccc(-c5ccc6c(c5)oc5ccccc56)c5oc6ccccc6c45)cc3)nc(-c3cccc4oc5ccccc5c34)n2)cc1 ; c1ccc(-c2nc(-c3ccc(-c4ccc(-c5ccc6c(c5)oc5ccccc56)c5oc6ccccc6c45)cc3)nc(-c3ccc4oc5ccccc5c4c3)n2)cc1]

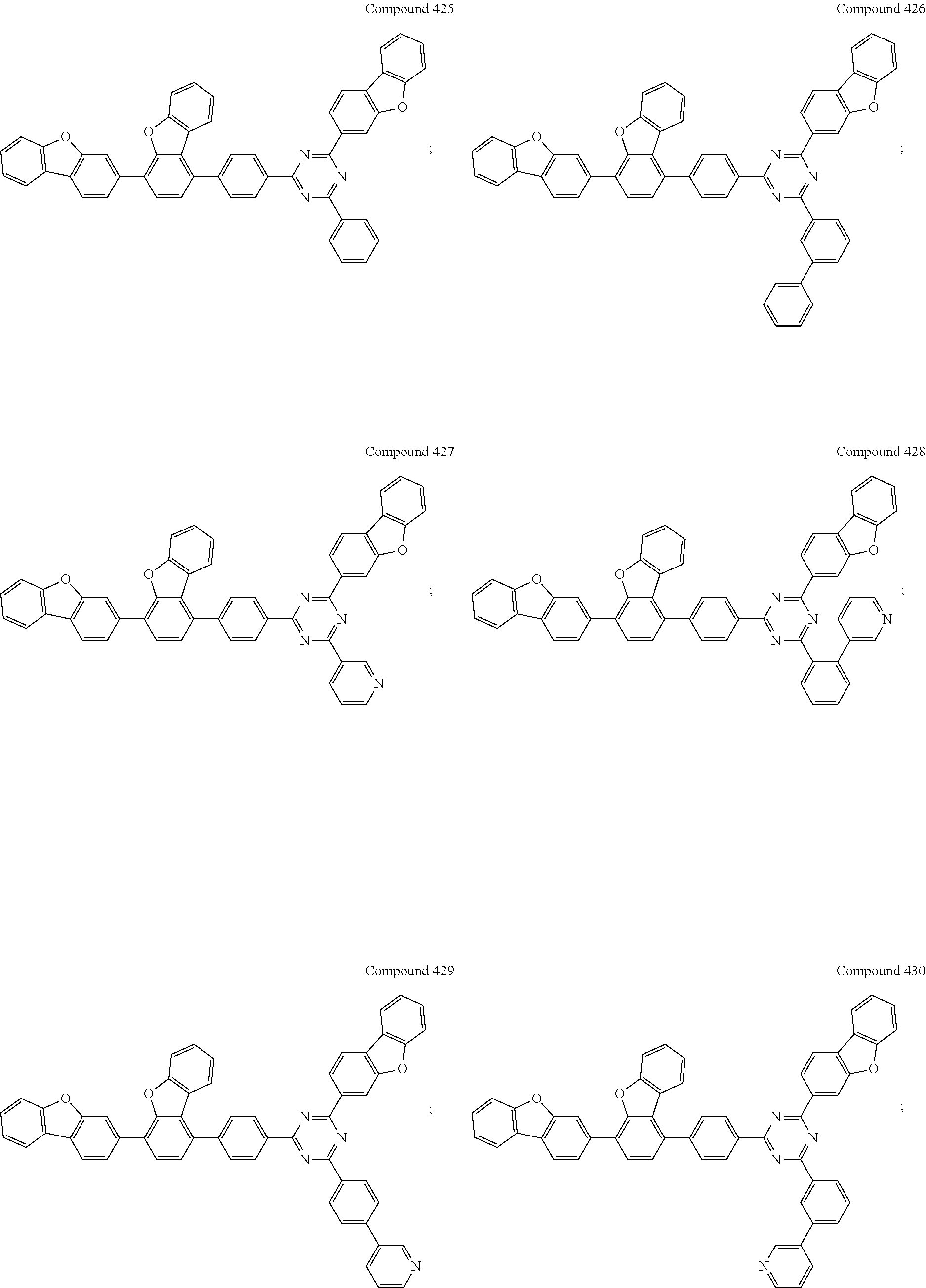

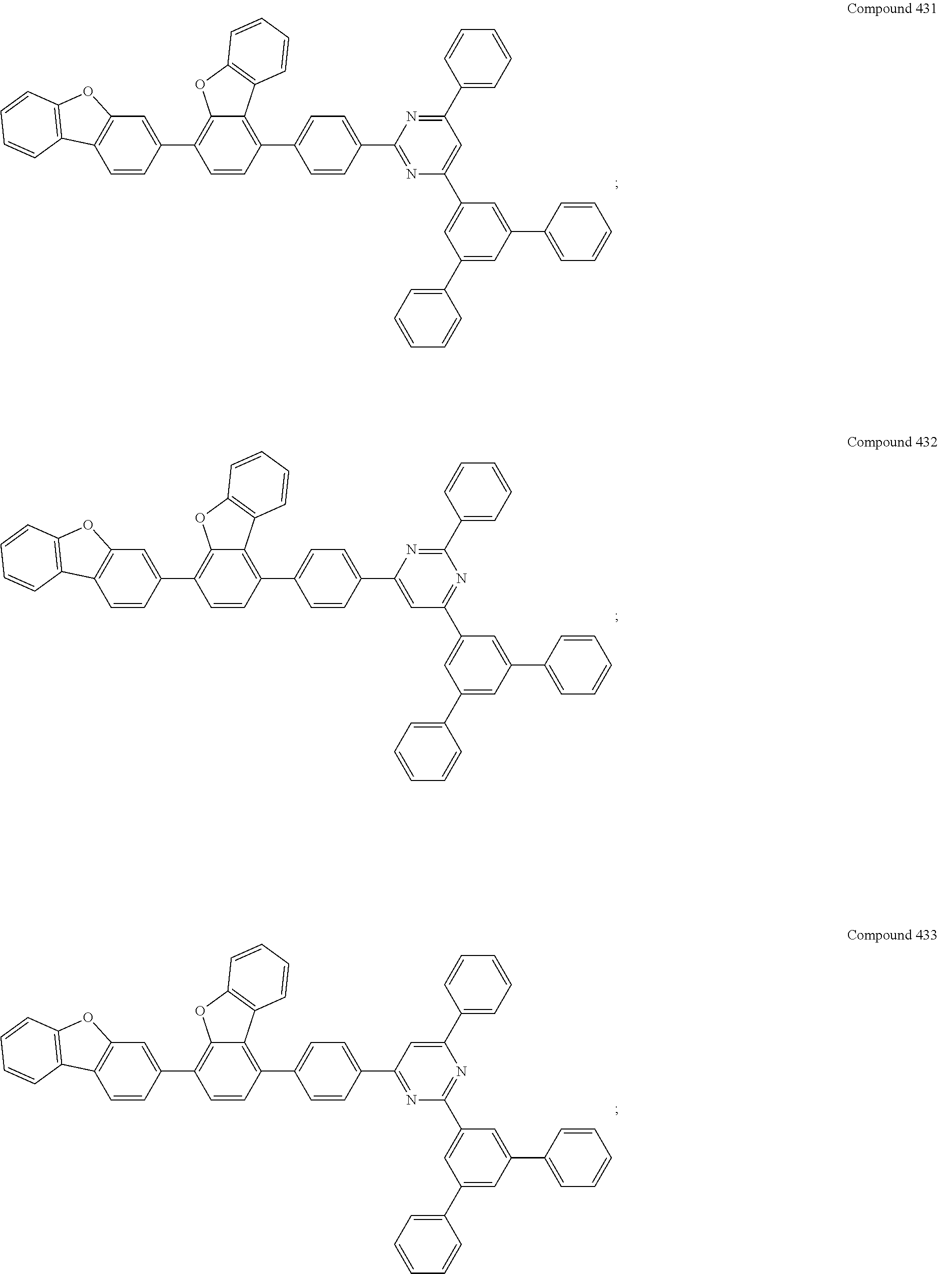 [SMILES: c1ccc(-c2cc(-c3ccccc3)cc(-c3cc(-c4ccccc4)nc(-c4ccc(-c5ccc(-c6ccc7c(c6)oc6ccccc67)c6oc7ccccc7c56)cc4)n3)c2)cc1 ; c1ccc(-c2cc(-c3ccccc3)cc(-c3cc(-c4ccc(-c5ccc(-c6ccc7c(c6)oc6ccccc67)c6oc7ccccc7c56)cc4)nc(-c4ccccc4)n3)c2)cc1 ; c1ccc(-c2cc(-c3ccccc3)cc(-c3nc(-c4ccccc4)cc(-c4ccc(-c5ccc(-c6ccc7c(c6)oc6ccccc67)c6oc7ccccc7c56)cc4)n3)c2)cc1]

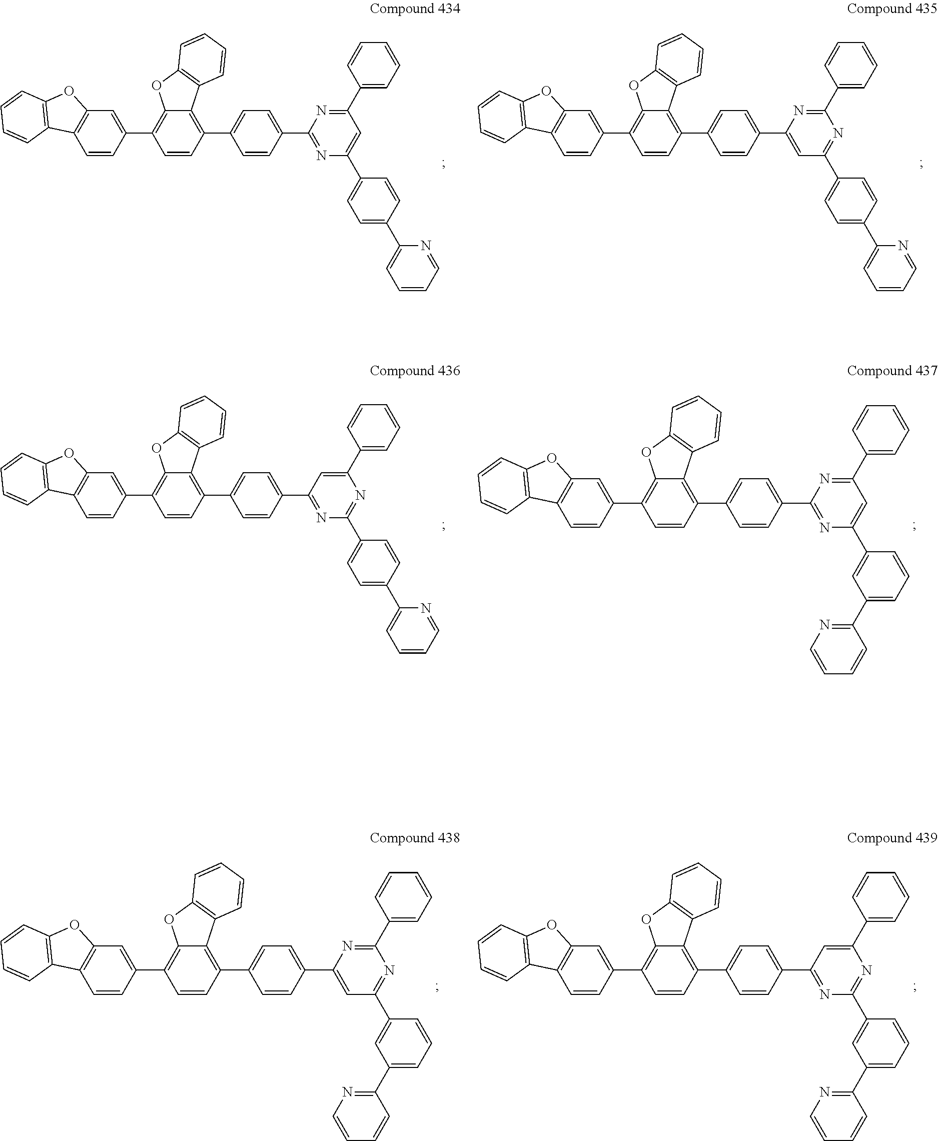

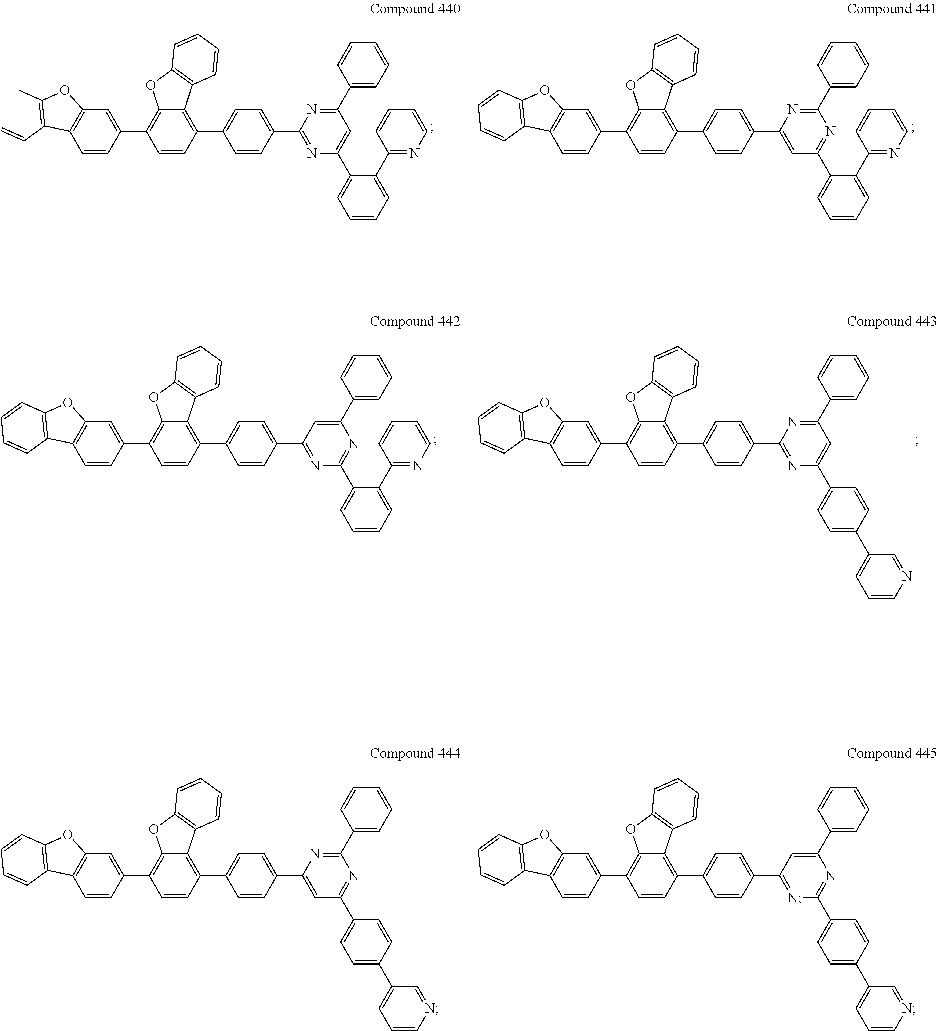

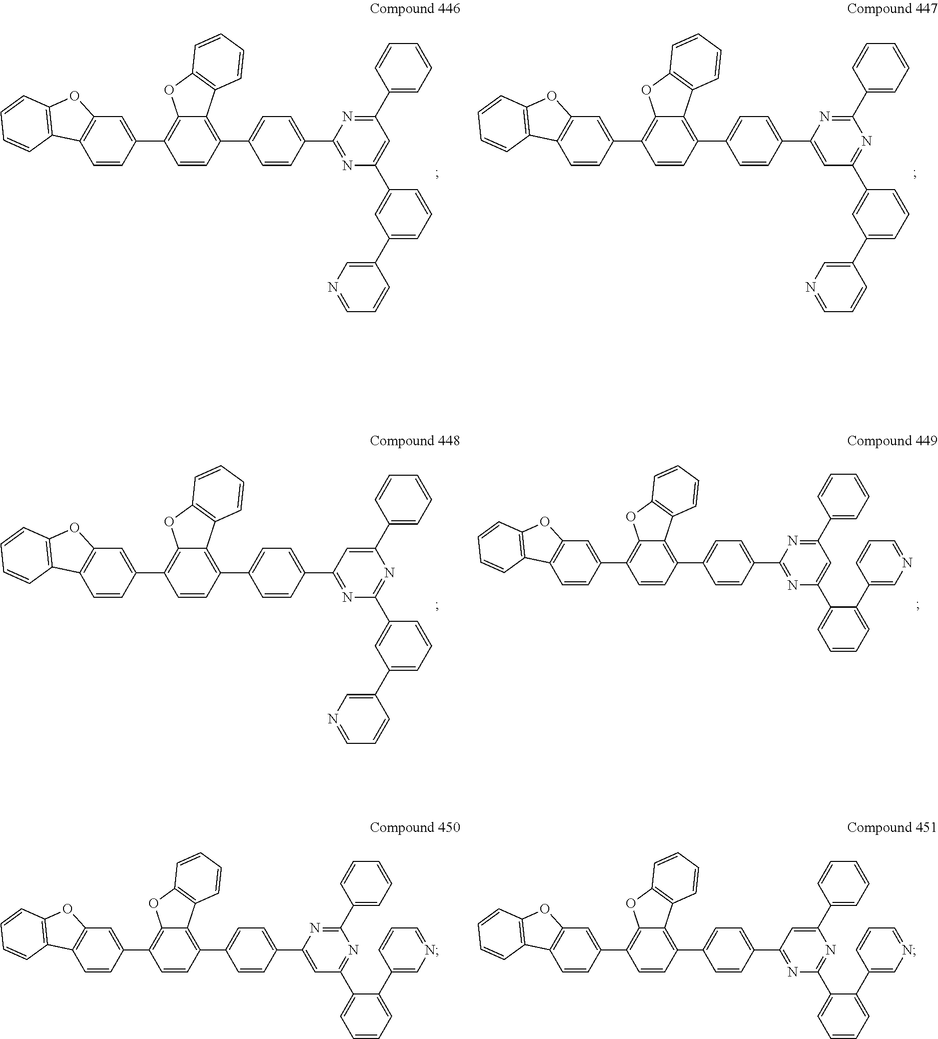

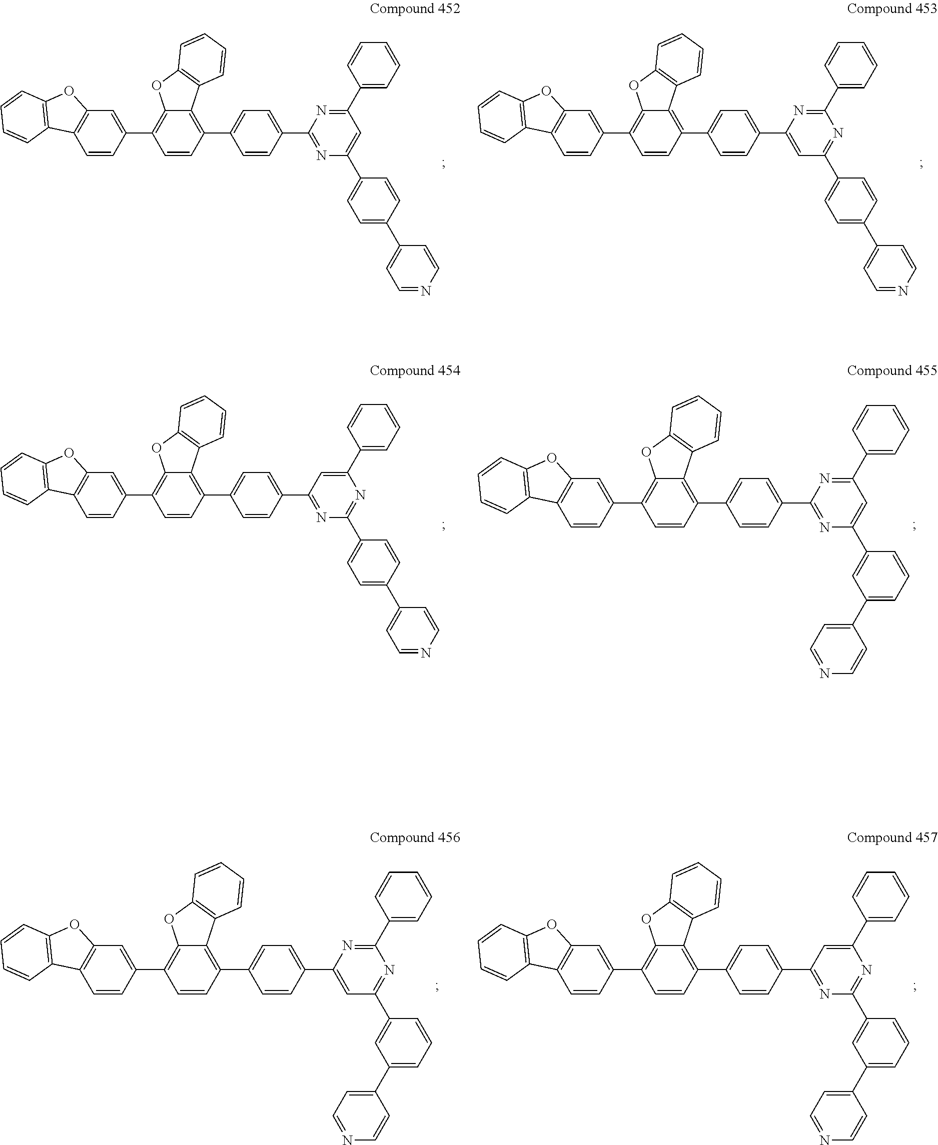

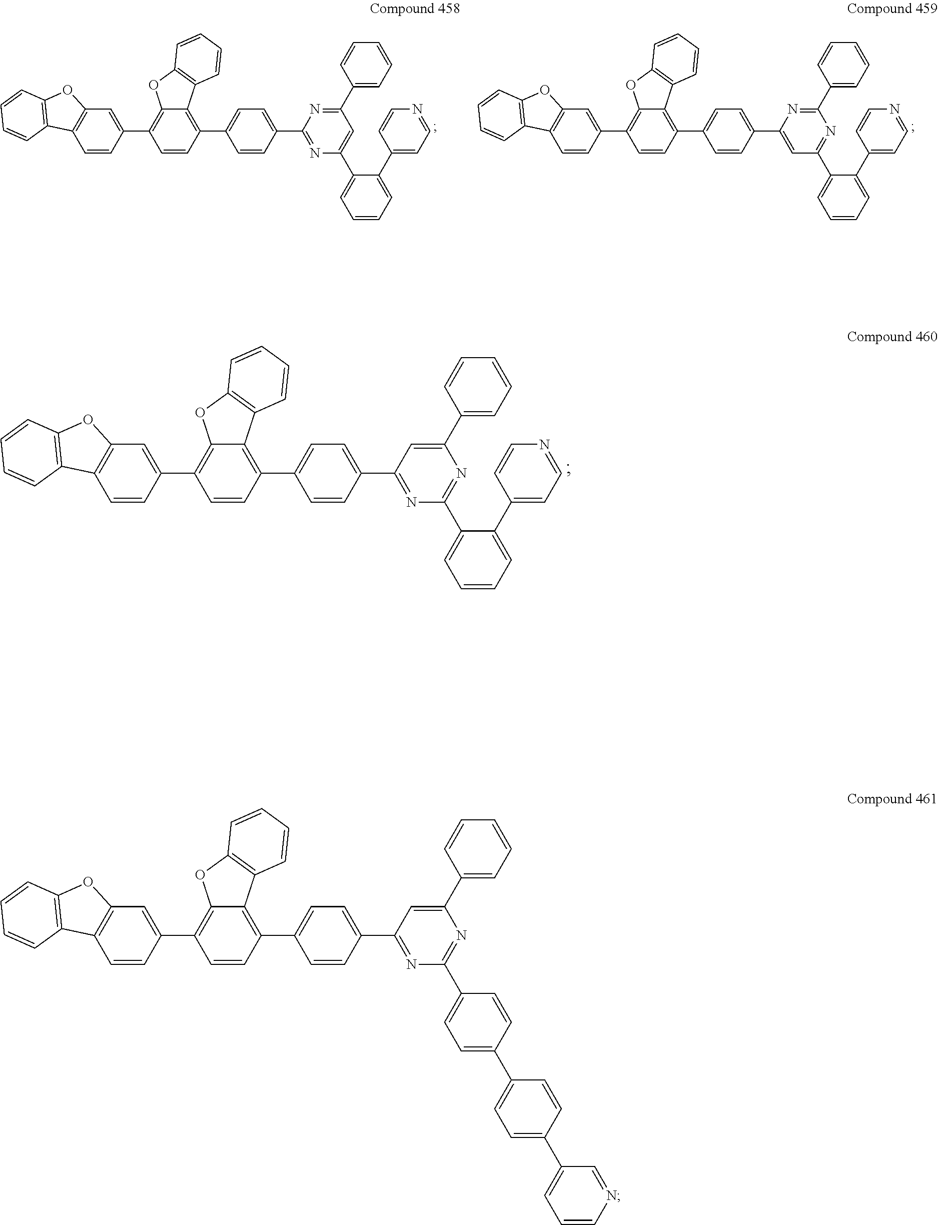 [SMILES: c1ccc(-c2cc(-c3ccccc3-c3ccncc3)nc(-c3ccc(-c4ccc(-c5ccc6c(c5)oc5ccccc56)c5oc6ccccc6c45)cc3)n2)cc1 ; c1ccc(-c2nc(-c3ccc(-c4ccc(-c5ccc6c(c5)oc5ccccc56)c5oc6ccccc6c45)cc3)cc(-c3ccccc3-c3ccncc3)n2)cc1 ; c1ccc(-c2cc(-c3ccc(-c4ccc(-c5ccc6c(c5)oc5ccccc56)c5oc6ccccc6c45)cc3)nc(-c3ccccc3-c3ccncc3)n2)cc1 ; c1ccc(-c2cc(-c3ccc(-c4ccc(-c5ccc6c(c5)oc5ccccc56)c5oc6ccccc6c45)cc3)nc(-c3ccc(-c4ccc(-c5cccnc5)cc4)cc3)n2)cc1]

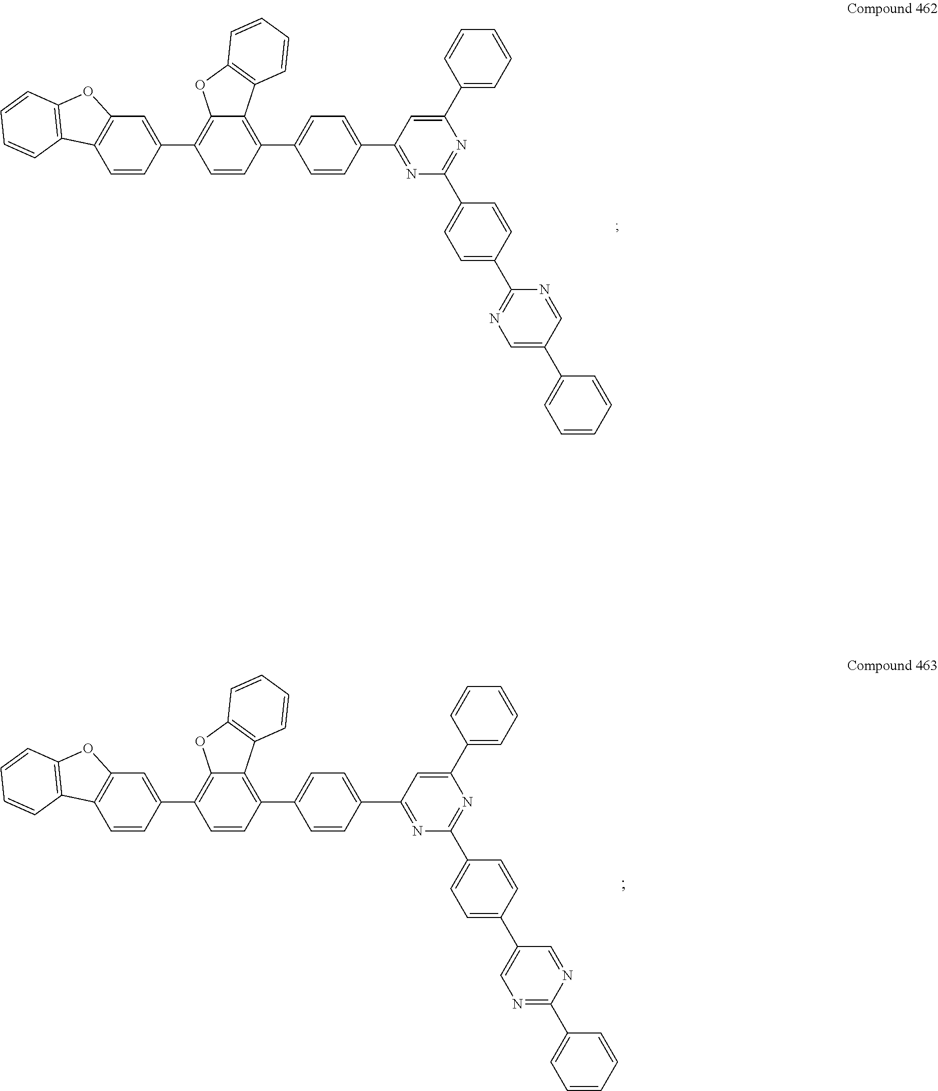 [SMILES: c1ccc(-c2cnc(-c3ccc(-c4nc(-c5ccccc5)cc(-c5ccc(-c6ccc(-c7ccc8c(c7)oc7ccccc78)c7oc8ccccc8c67)cc5)n4)cc3)nc2)cc1 ; c1ccc(-c2cc(-c3ccc(-c4ccc(-c5ccc6c(c5)oc5ccccc56)c5oc6ccccc6c45)cc3)nc(-c3ccc(-c4cnc(-c5ccccc5)nc4)cc3)n2)cc1]

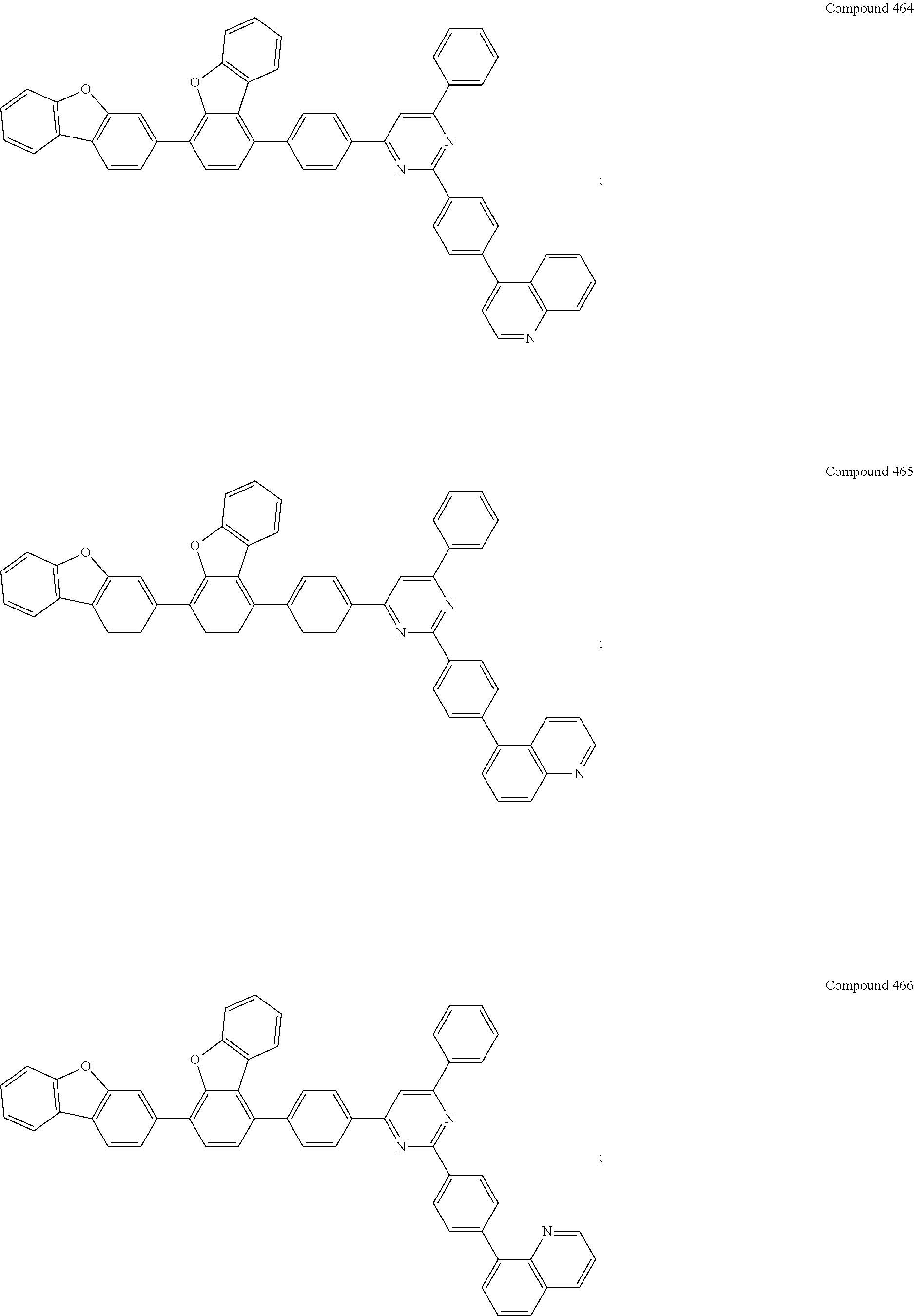

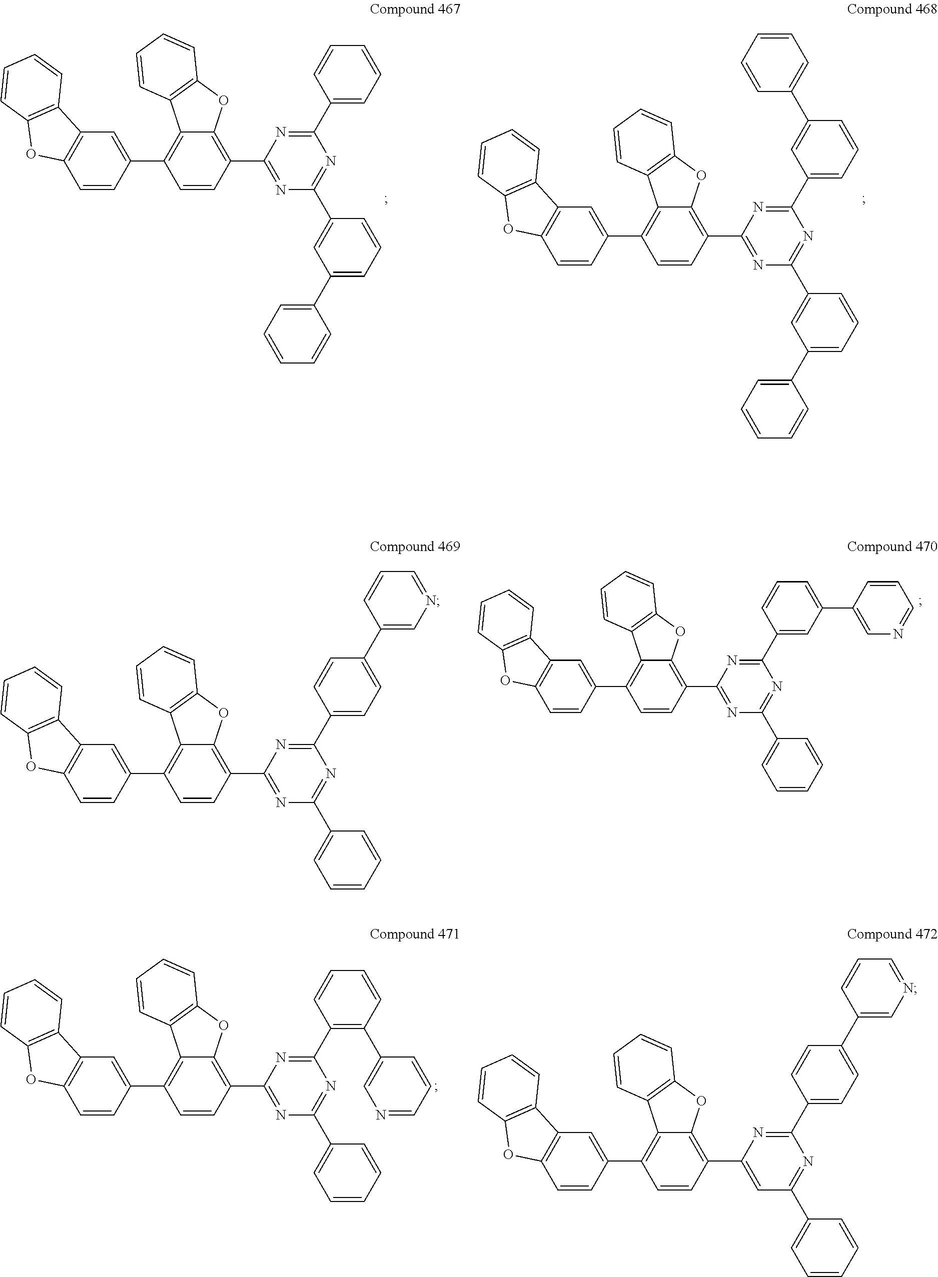

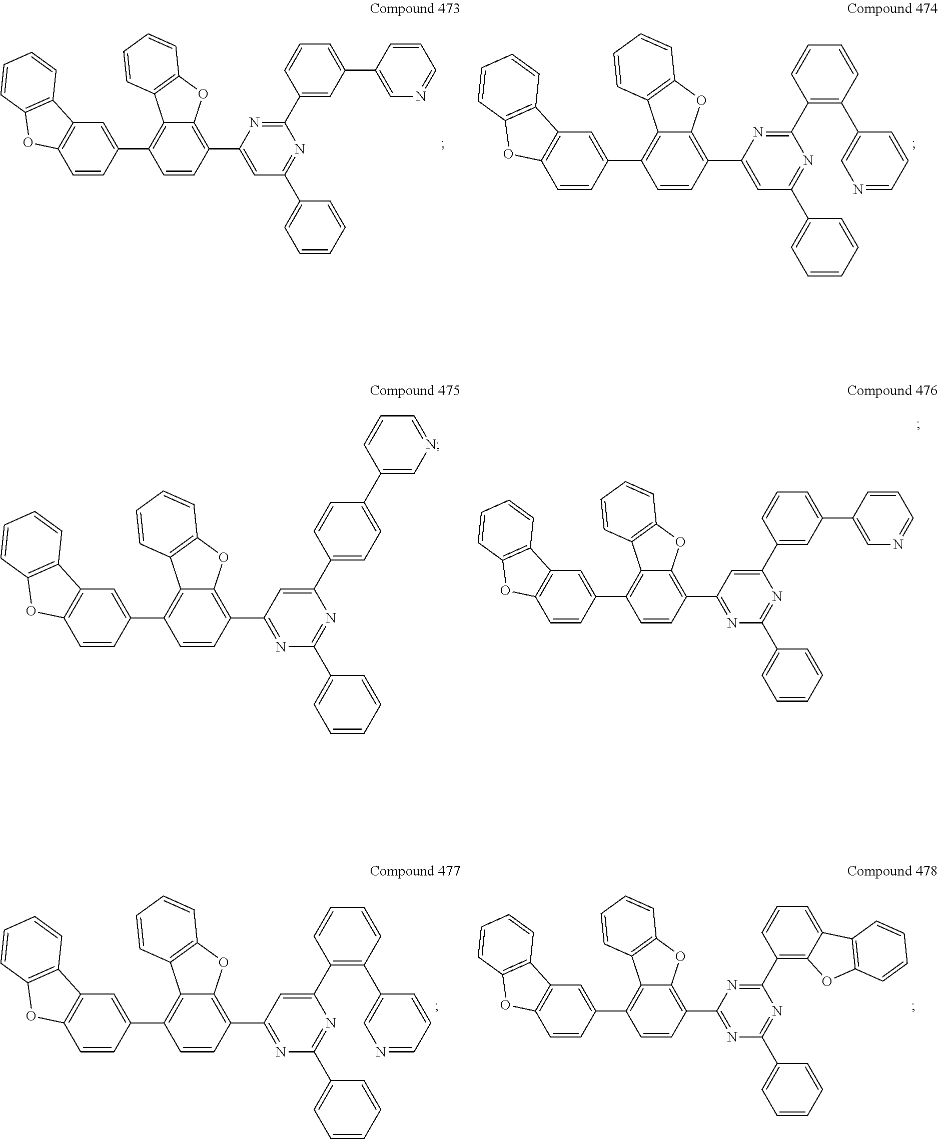

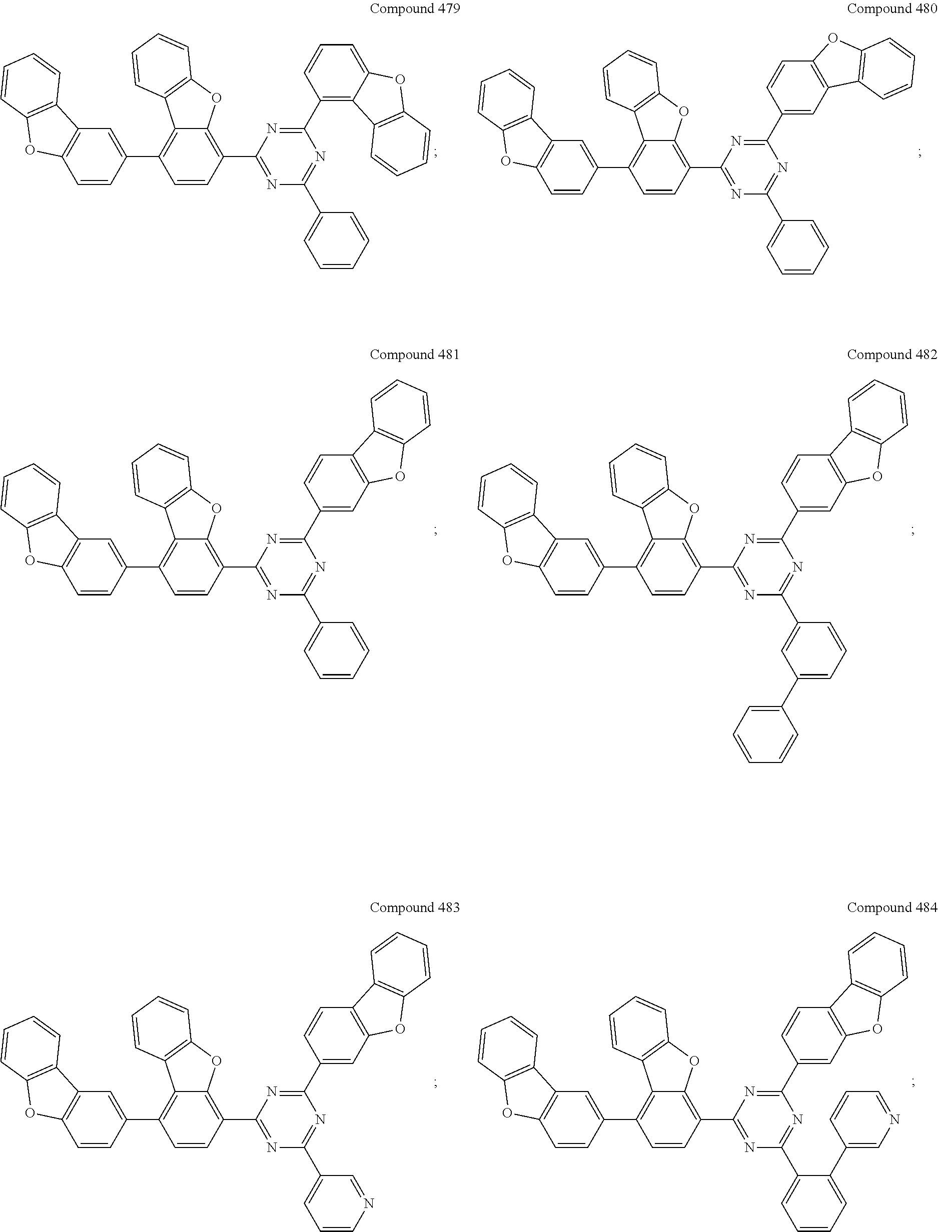

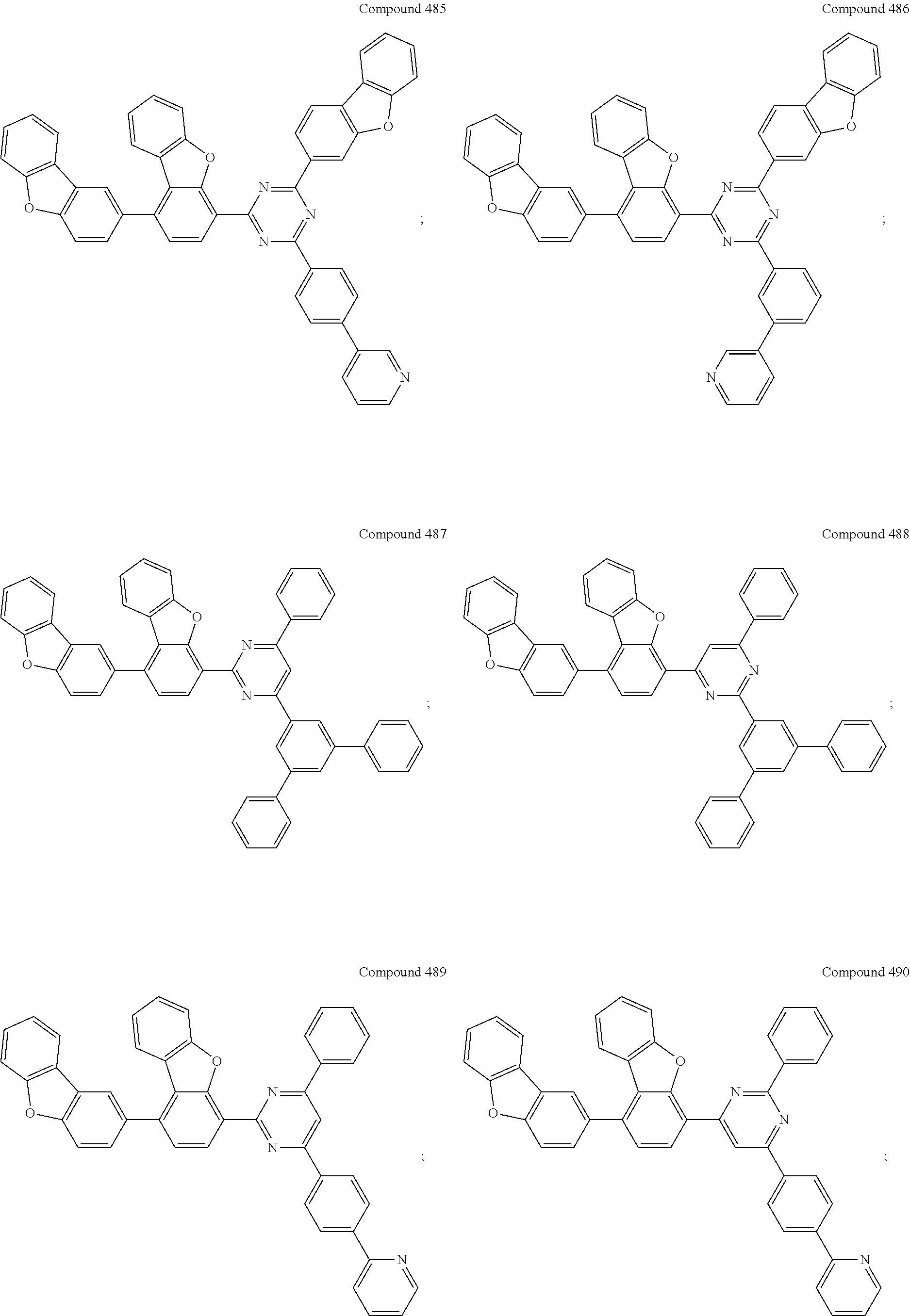

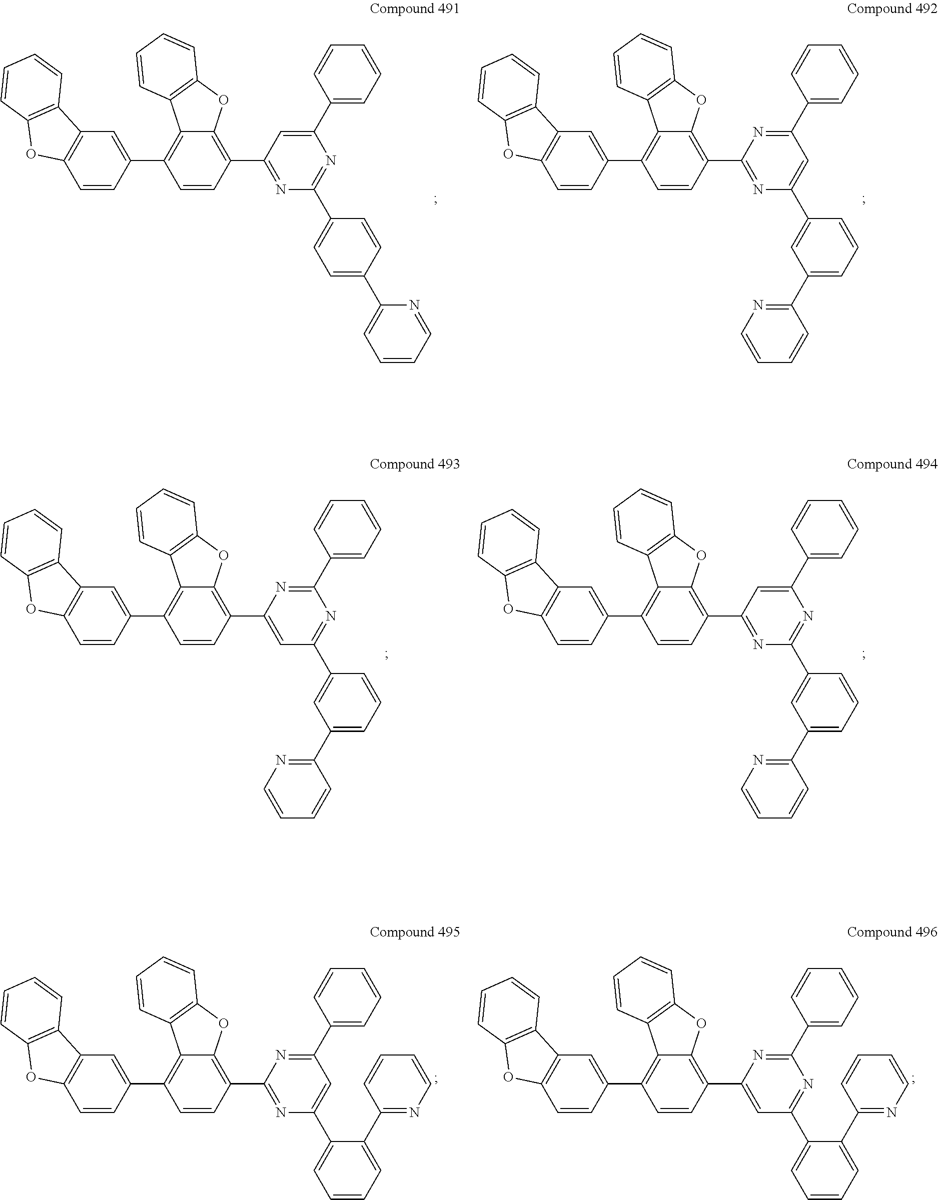

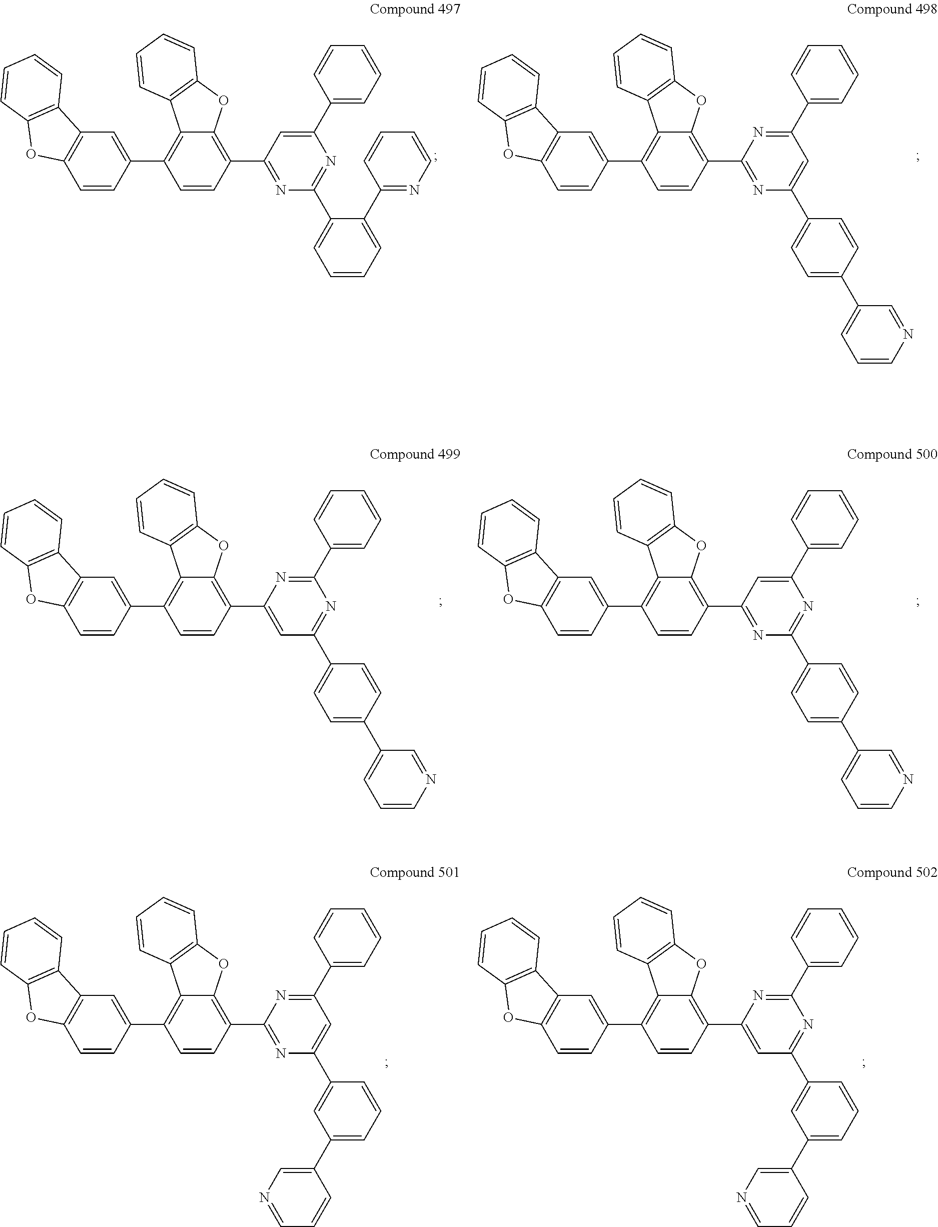

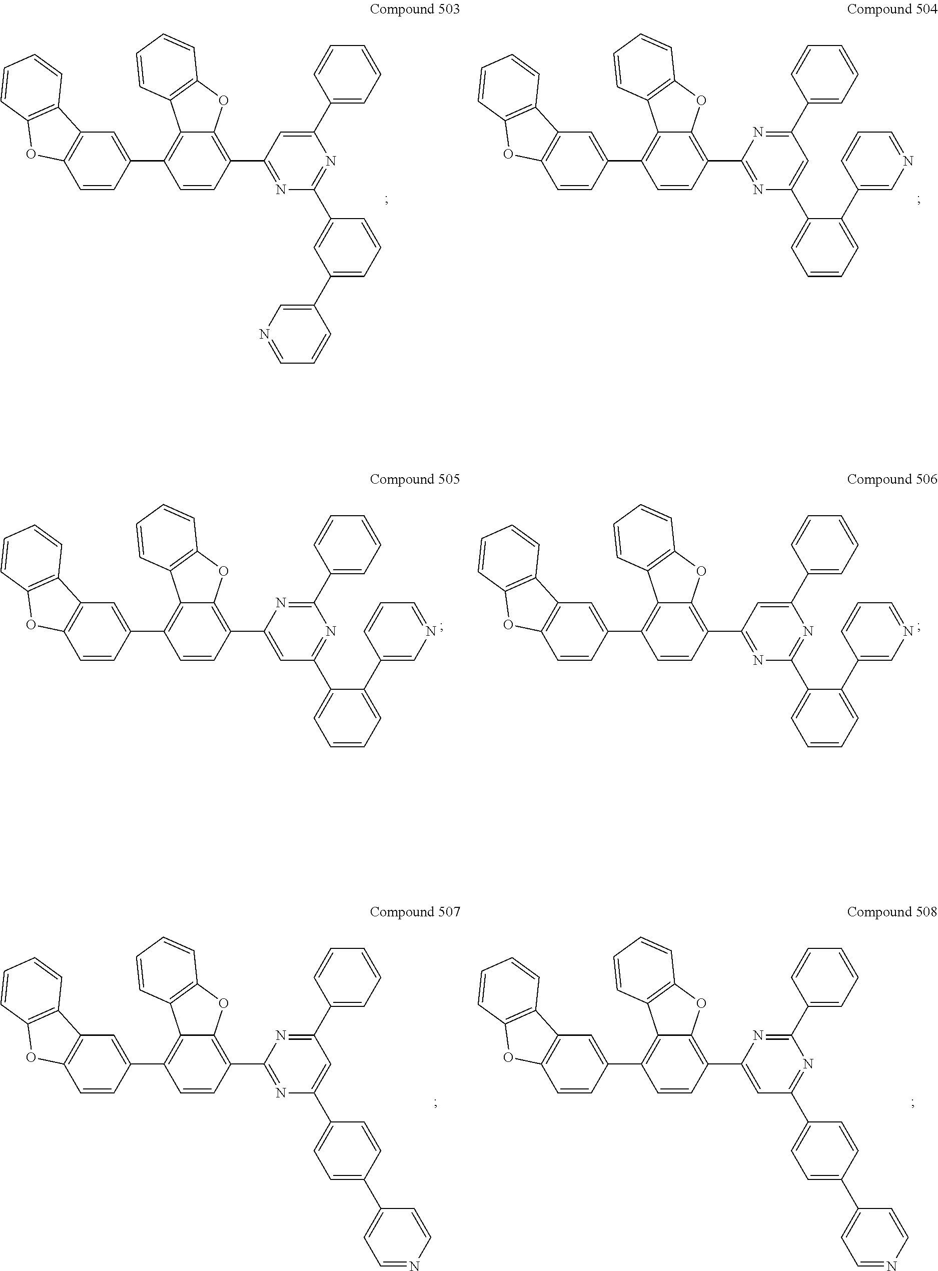

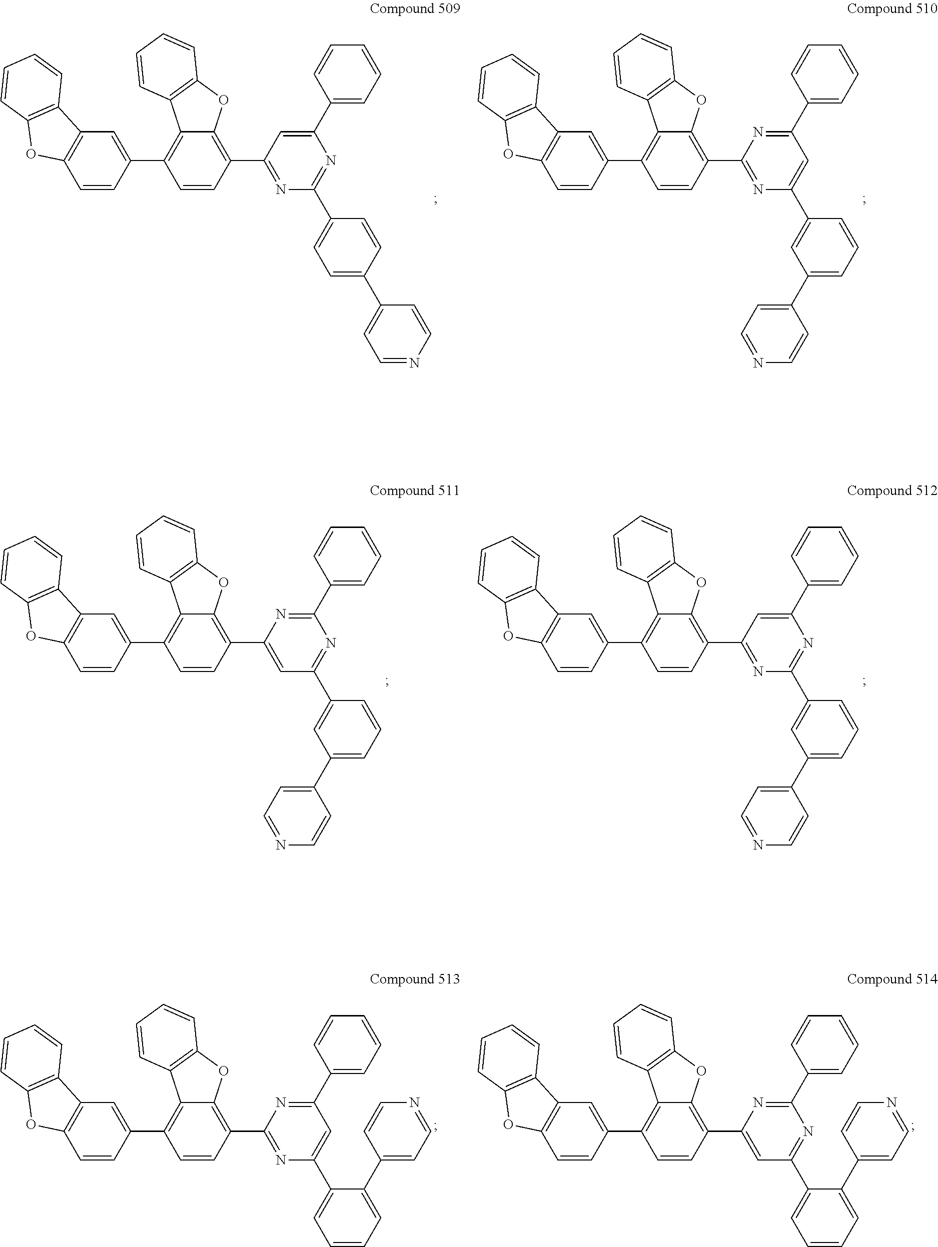

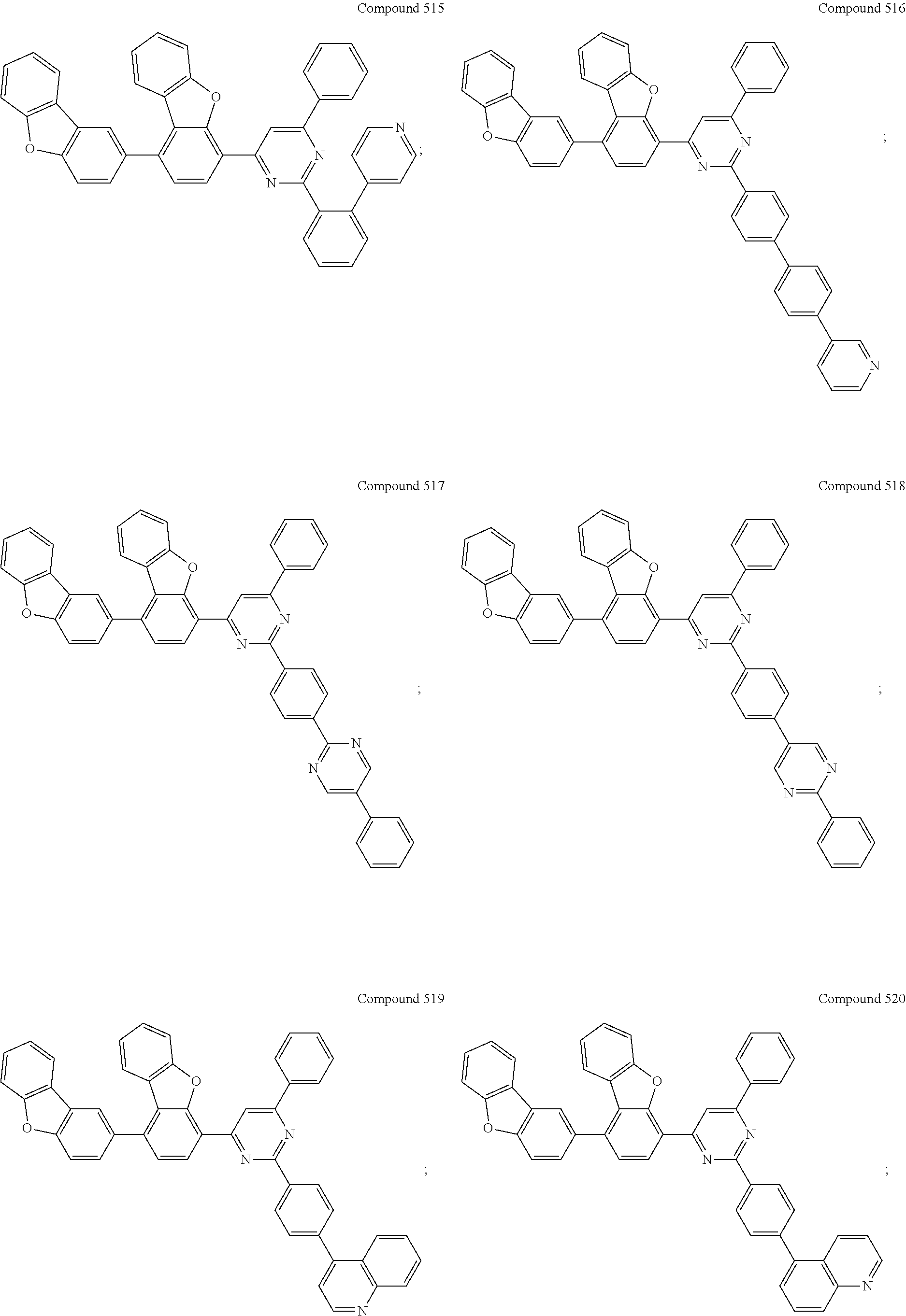

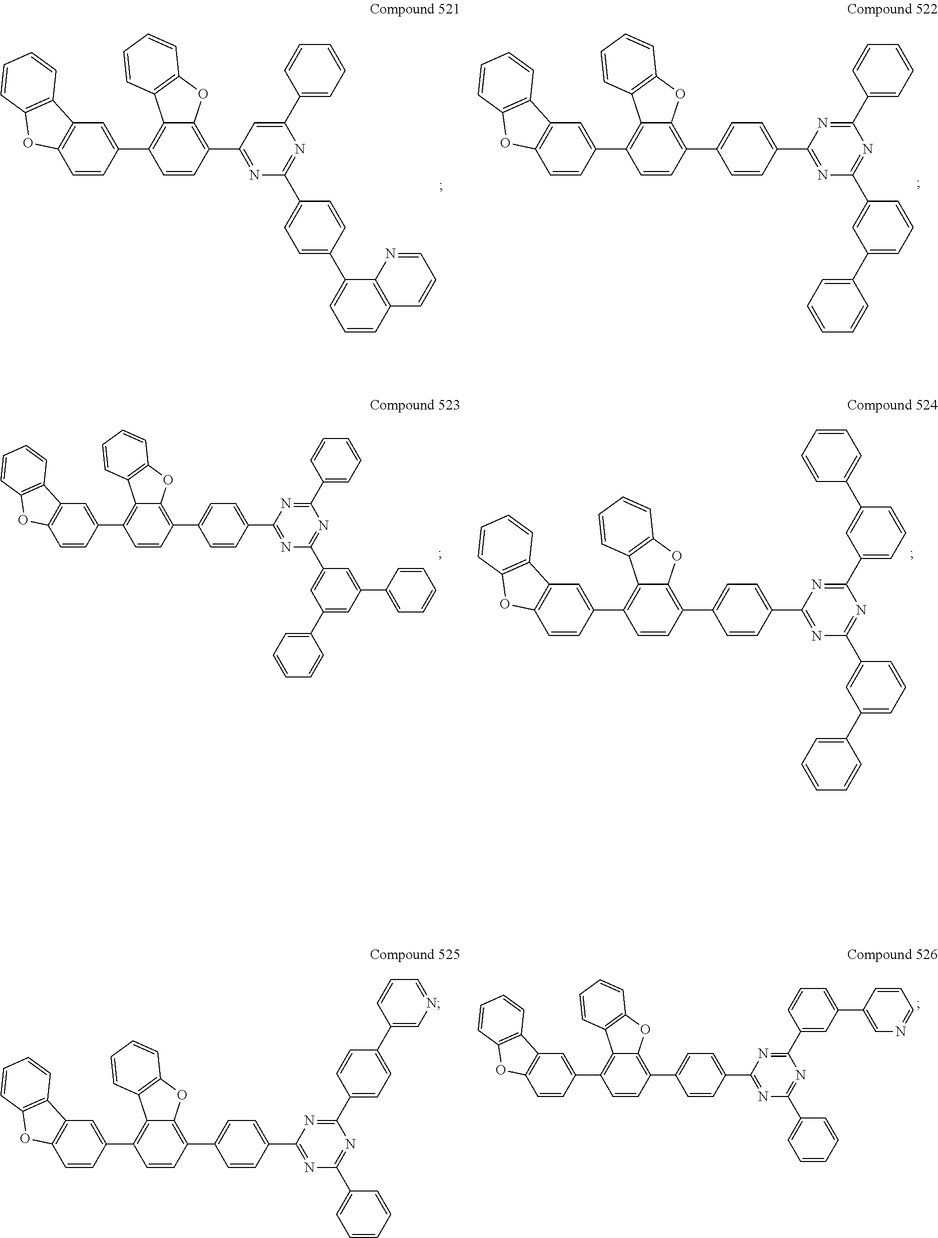

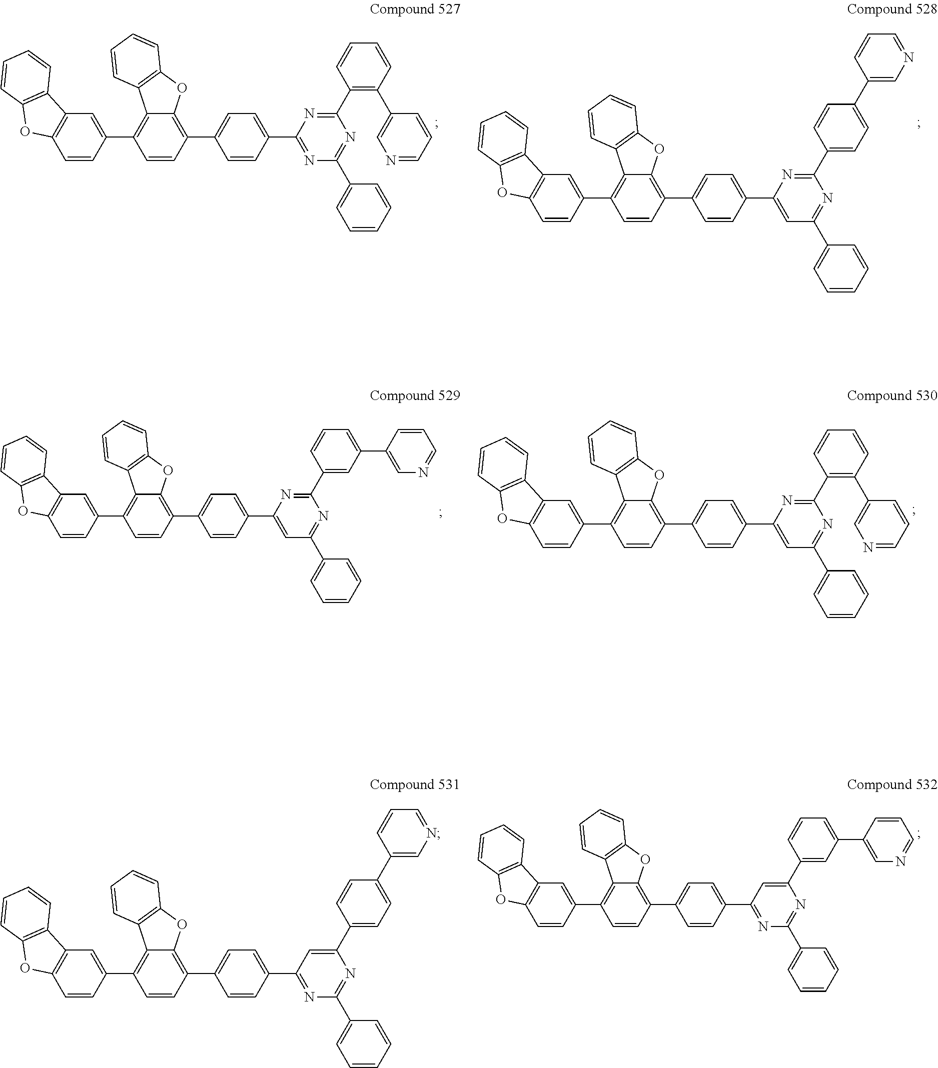

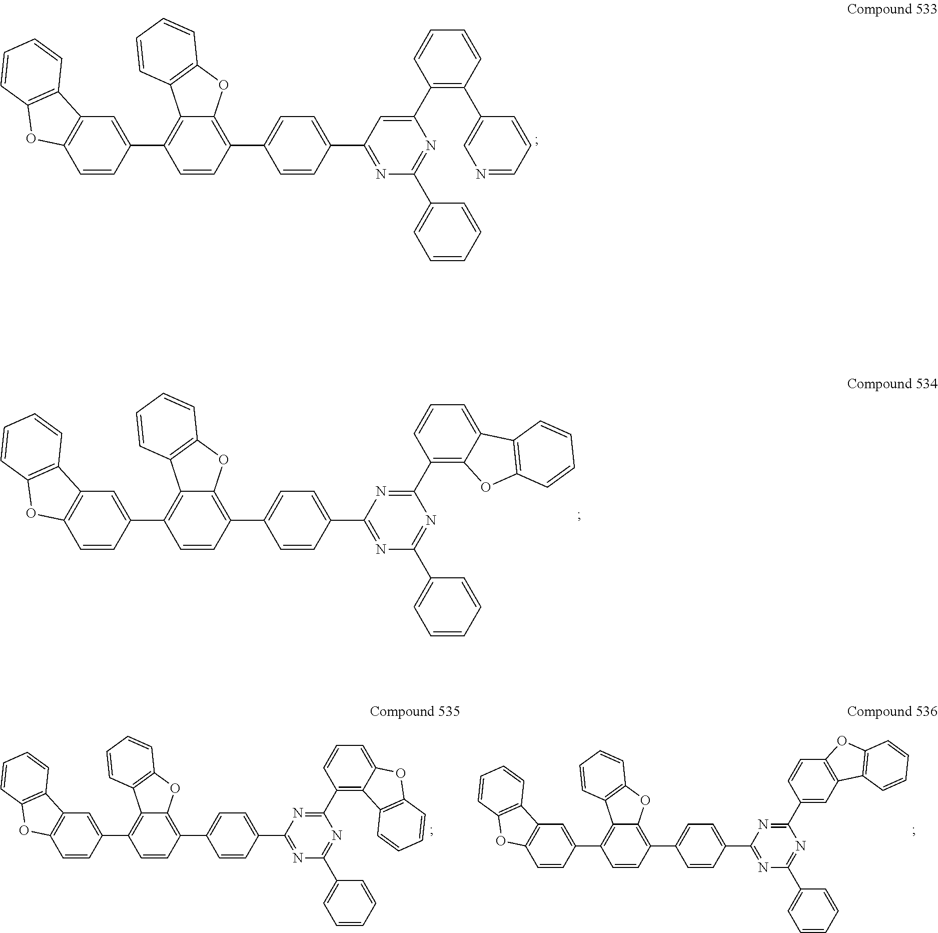 [SMILES: c1ccc(-c2nc(-c3ccc(-c4ccc(-c5ccc6oc7ccccc7c6c5)c5c4oc4ccccc45)cc3)cc(-c3ccccc3-c3cccnc3)n2)cc1 ; c1ccc(-c2nc(-c3ccc(-c4ccc(-c5ccc6oc7ccccc7c6c5)c5c4oc4ccccc45)cc3)nc(-c3cccc4c3oc3ccccc34)n2)cc1 ; c1ccc(-c2nc(-c3ccc(-c4ccc(-c5ccc6oc7ccccc7c6c5)c5c4oc4ccccc45)cc3)nc(-c3cccc4oc5ccccc5c34)n2)cc1 ; c1ccc(-c2nc(-c3ccc(-c4ccc(-c5ccc6oc7ccccc7c6c5)c5c4oc4ccccc45)cc3)nc(-c3ccc4oc5ccccc5c4c3)n2)cc1]

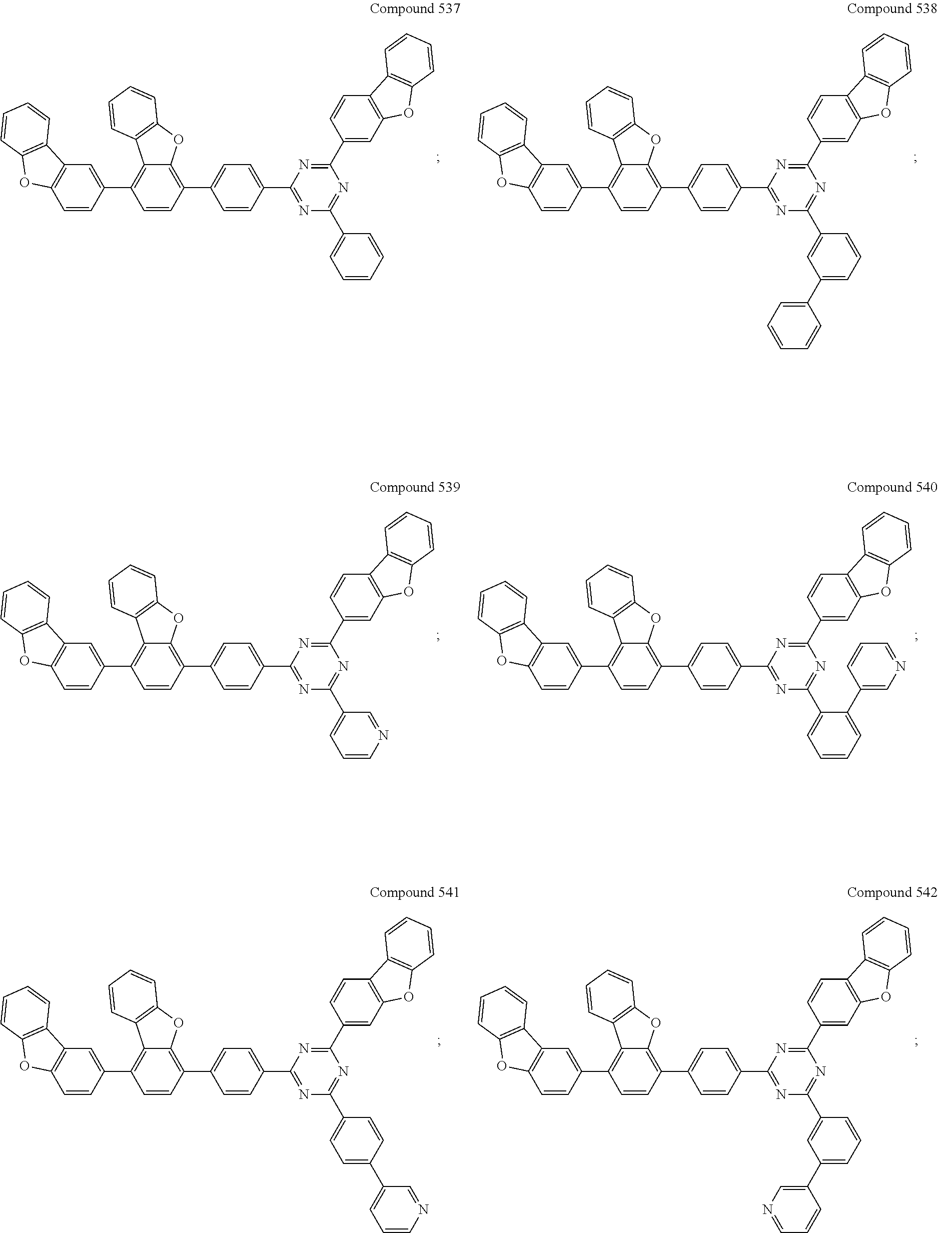

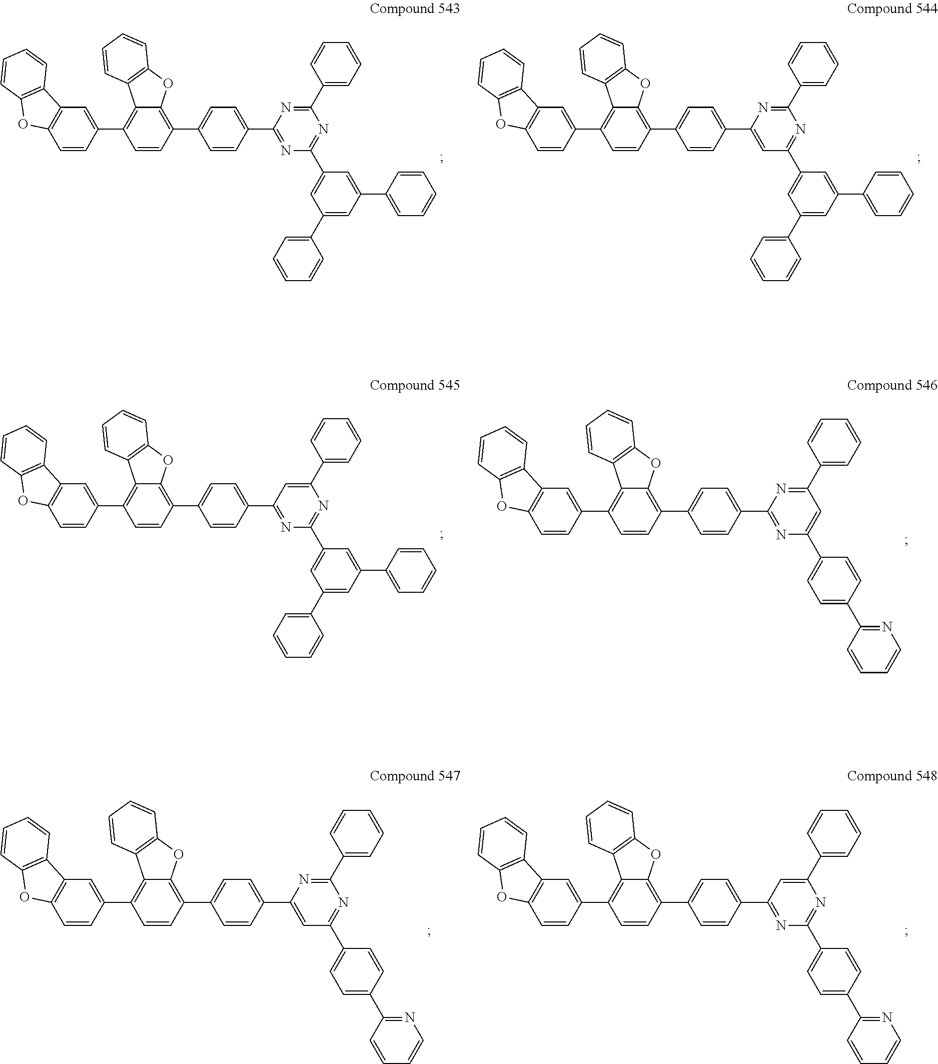

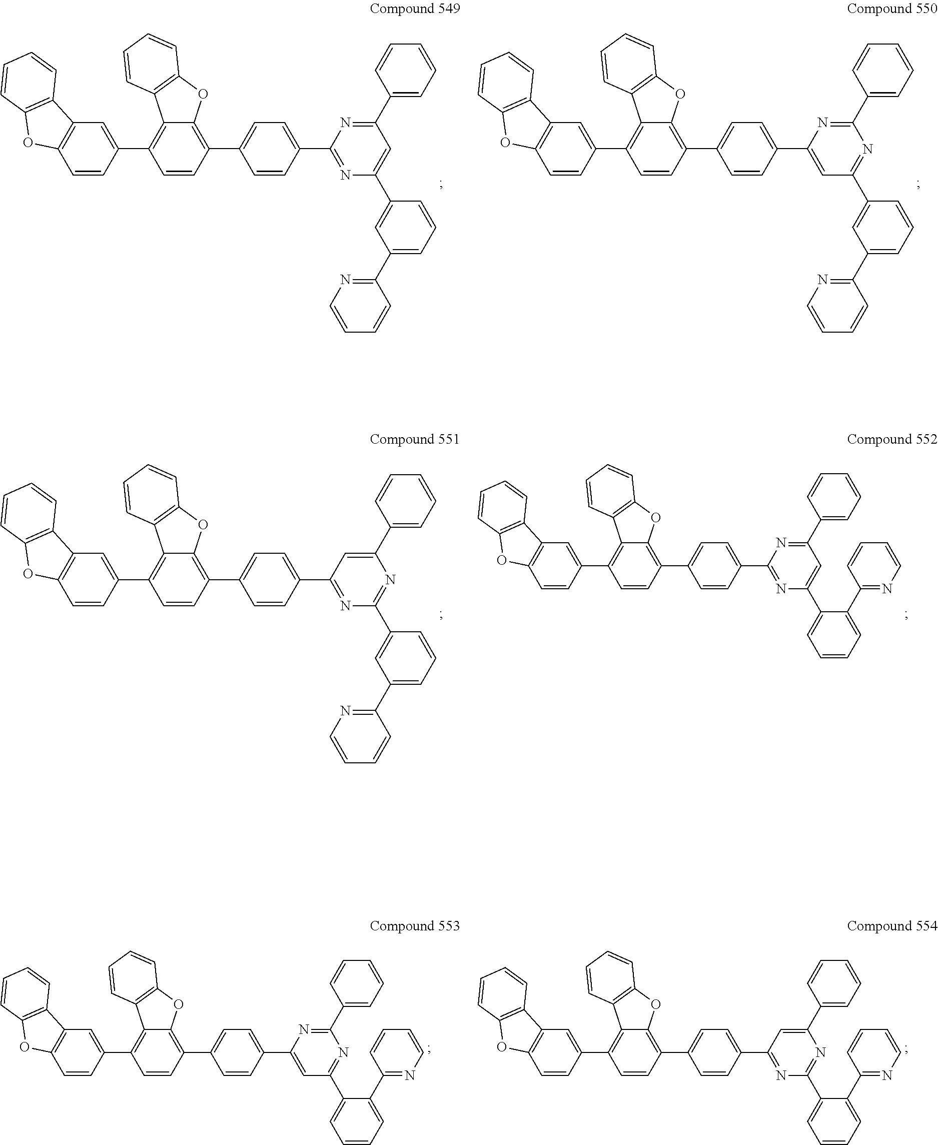

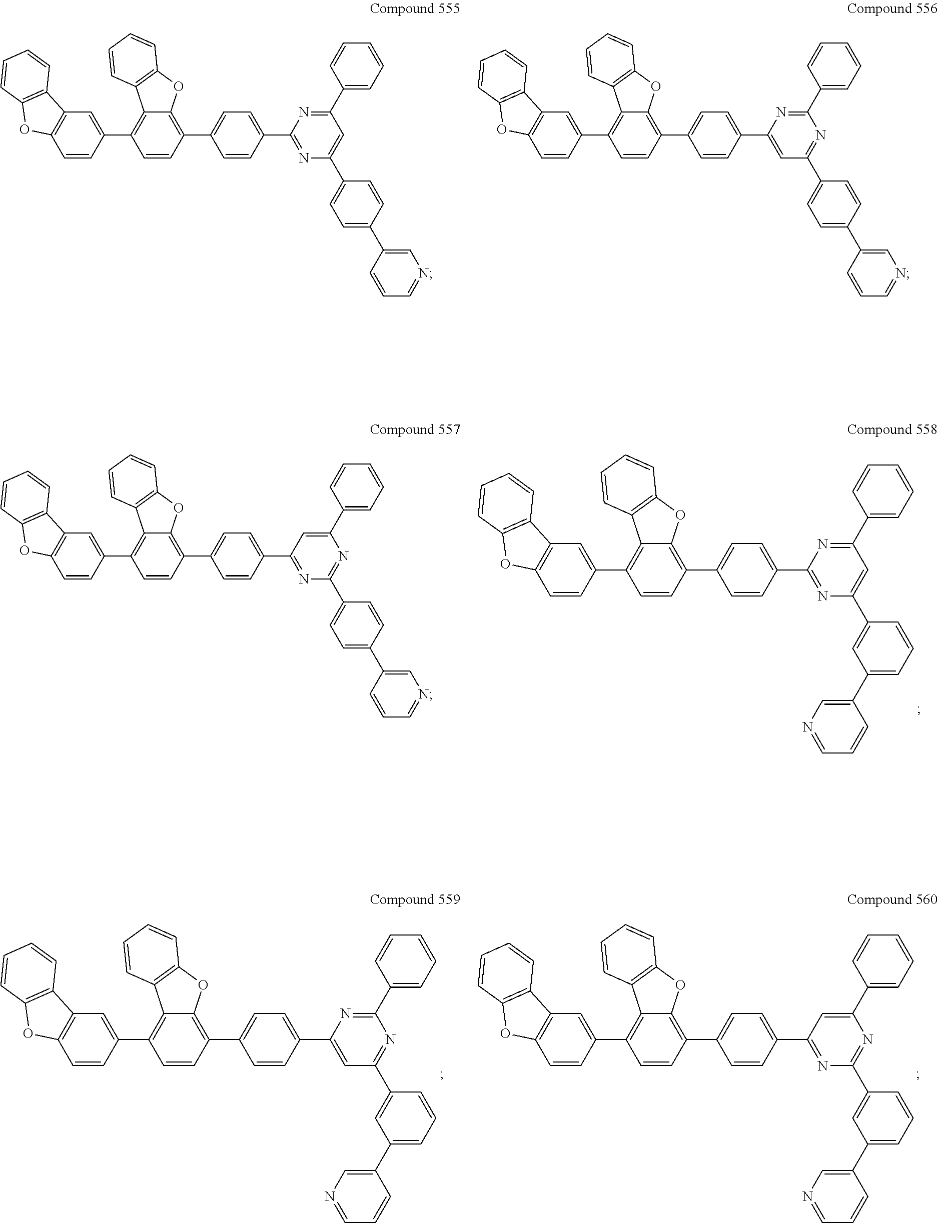

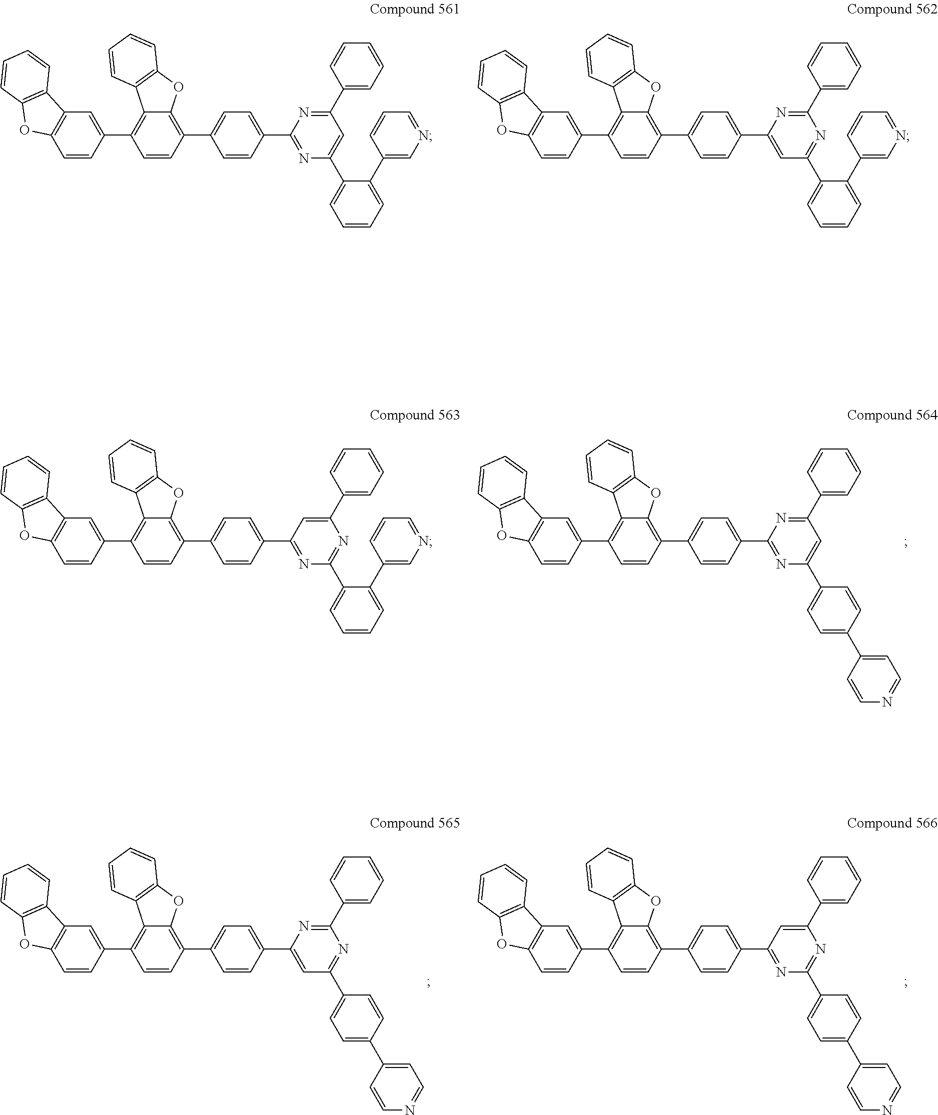

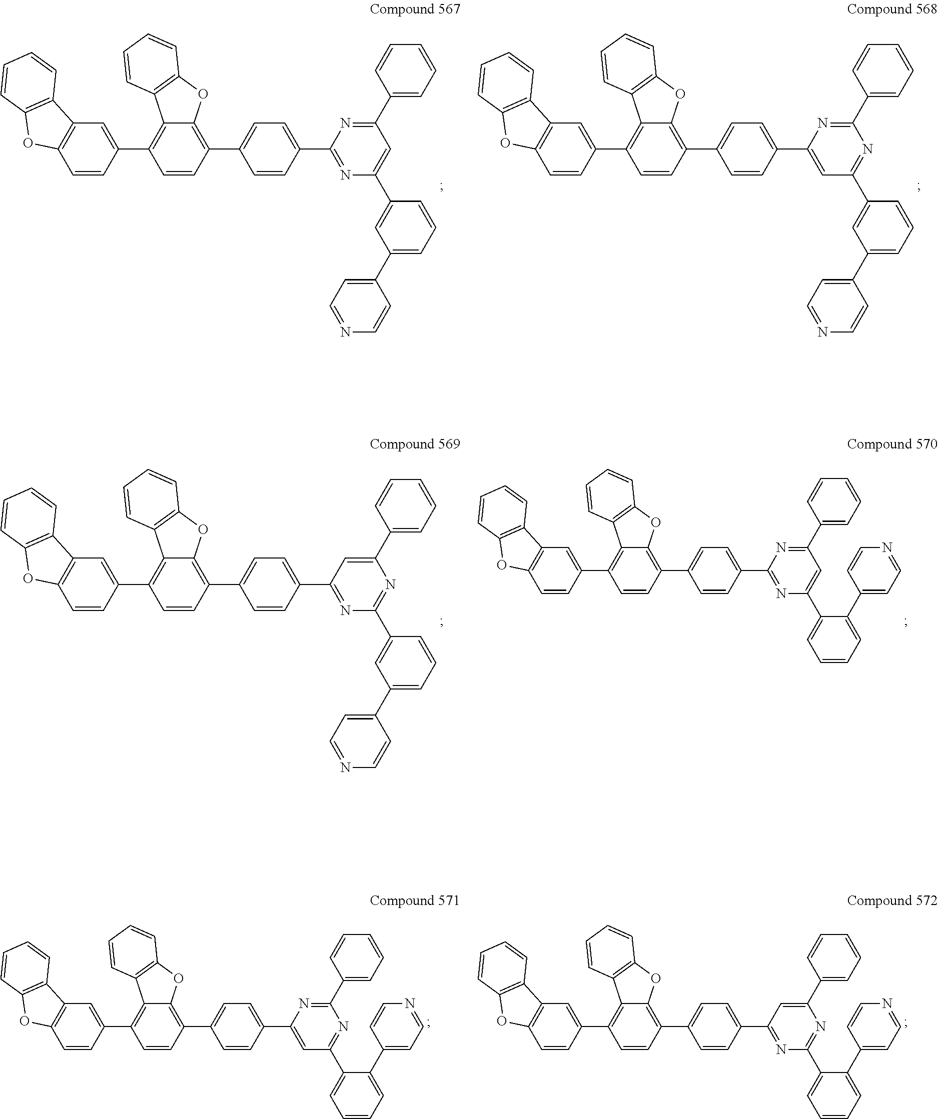

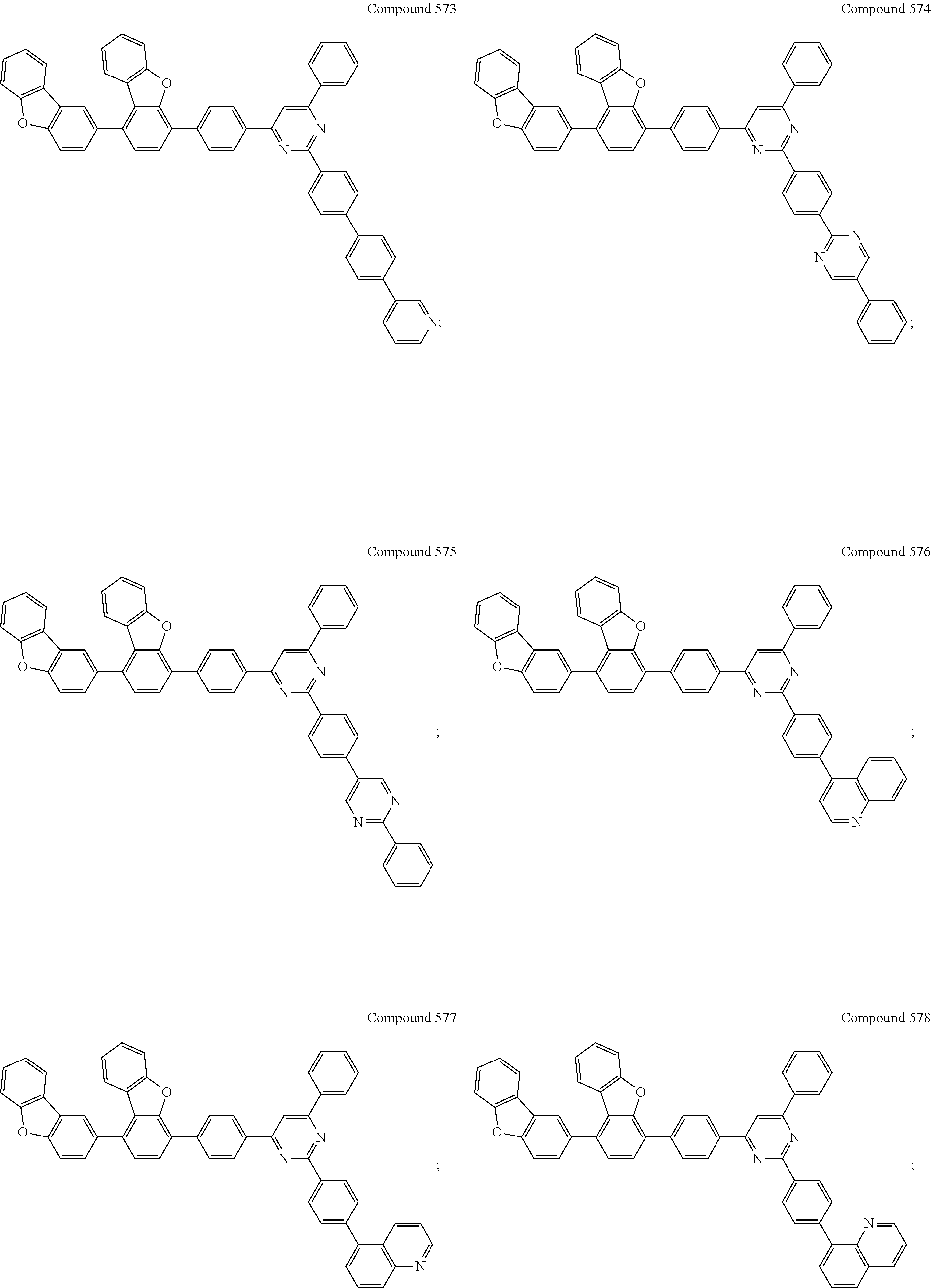

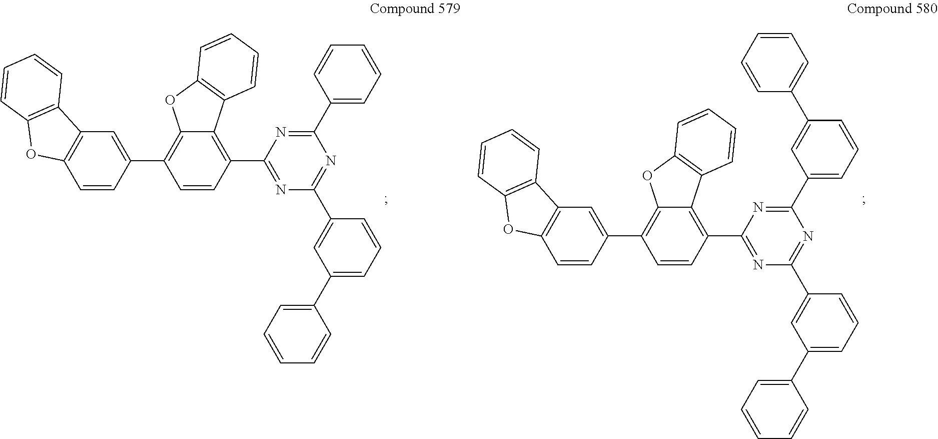 [SMILES: c1ccc(-c2cccc(-c3nc(-c4ccccc4)nc(-c4ccc(-c5ccc6oc7ccccc7c6c5)c5oc6ccccc6c45)n3)c2)cc1 ; c1ccc(-c2cccc(-c3nc(-c4cccc(-c5ccccc5)c4)nc(-c4ccc(-c5ccc6oc7ccccc7c6c5)c5oc6ccccc6c45)n3)c2)cc1]

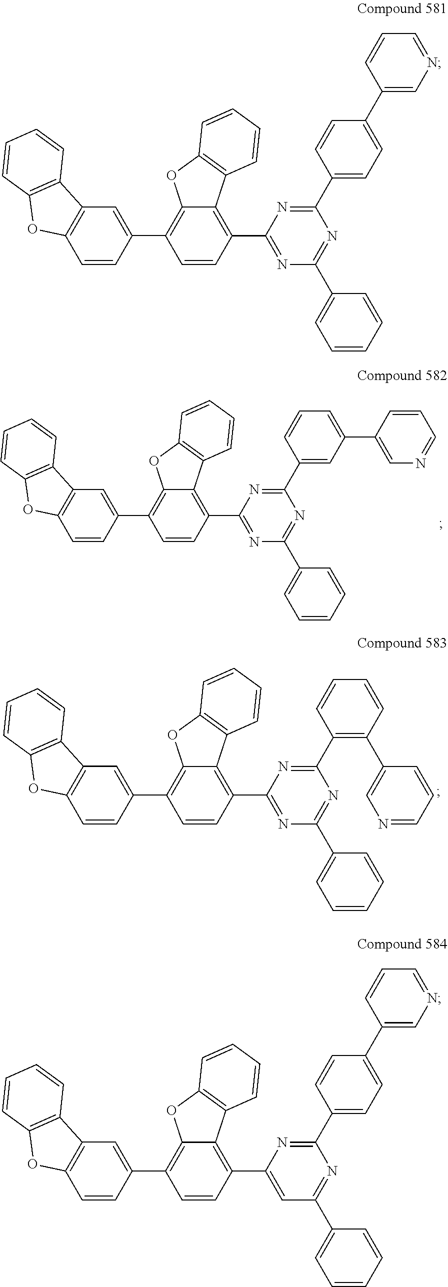 [SMILES: c1ccc(-c2nc(-c3ccc(-c4cccnc4)cc3)nc(-c3ccc(-c4ccc5oc6ccccc6c5c4)c4oc5ccccc5c34)n2)cc1 ; c1ccc(-c2nc(-c3cccc(-c4cccnc4)c3)nc(-c3ccc(-c4ccc5oc6ccccc6c5c4)c4oc5ccccc5c34)n2)cc1 ; c1ccc(-c2nc(-c3ccccc3-c3cccnc3)nc(-c3ccc(-c4ccc5oc6ccccc6c5c4)c4oc5ccccc5c34)n2)cc1 ; c1ccc(-c2cc(-c3ccc(-c4ccc5oc6ccccc6c5c4)c4oc5ccccc5c34)nc(-c3ccc(-c4cccnc4)cc3)n2)cc1]

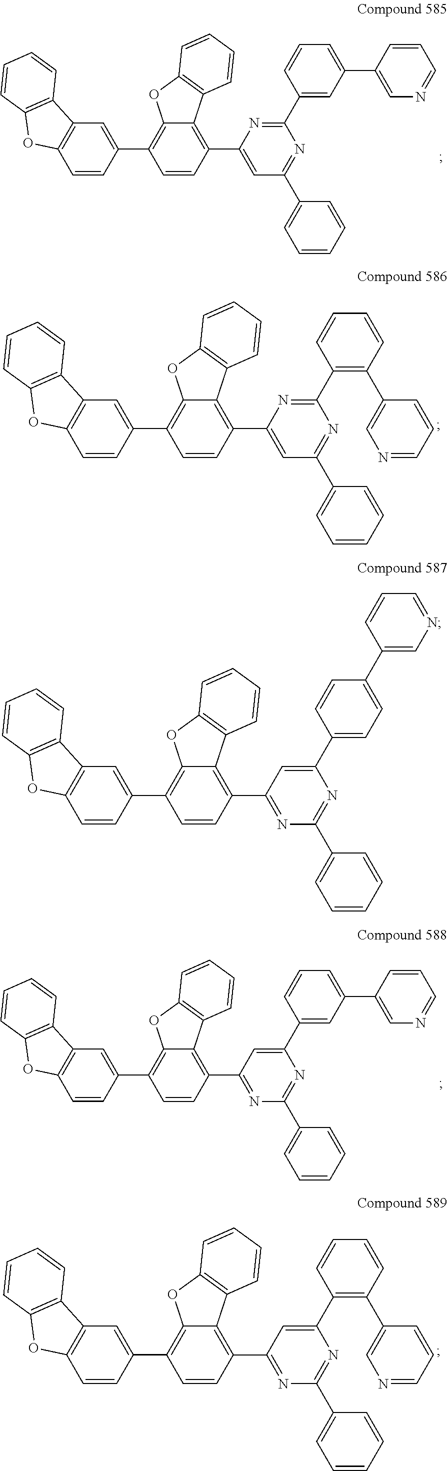 [SMILES: c1ccc(-c2cc(-c3ccc(-c4ccc5oc6ccccc6c5c4)c4oc5ccccc5c34)nc(-c3cccc(-c4cccnc4)c3)n2)cc1 ; c1ccc(-c2cc(-c3ccc(-c4ccc5oc6ccccc6c5c4)c4oc5ccccc5c34)nc(-c3ccccc3-c3cccnc3)n2)cc1 ; c1ccc(-c2nc(-c3ccc(-c4cccnc4)cc3)cc(-c3ccc(-c4ccc5oc6ccccc6c5c4)c4oc5ccccc5c34)n2)cc1 ; c1ccc(-c2nc(-c3cccc(-c4cccnc4)c3)cc(-c3ccc(-c4ccc5oc6ccccc6c5c4)c4oc5ccccc5c34)n2)cc1 ; c1ccc(-c2nc(-c3ccccc3-c3cccnc3)cc(-c3ccc(-c4ccc5oc6ccccc6c5c4)c4oc5ccccc5c34)n2)cc1]

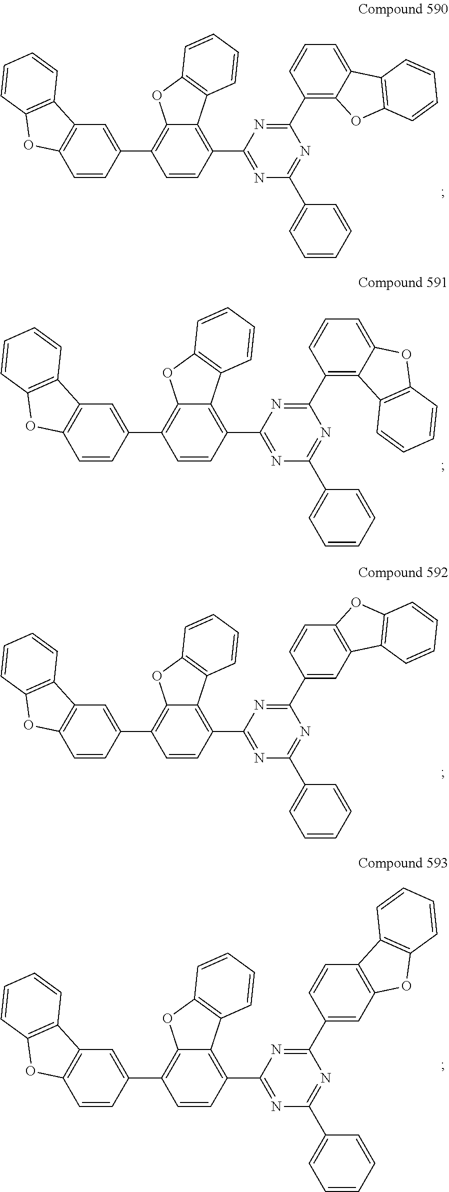 [SMILES: c1ccc(-c2nc(-c3cccc4c3oc3ccccc34)nc(-c3ccc(-c4ccc5oc6ccccc6c5c4)c4oc5ccccc5c34)n2)cc1 ; c1ccc(-c2nc(-c3cccc4oc5ccccc5c34)nc(-c3ccc(-c4ccc5oc6ccccc6c5c4)c4oc5ccccc5c34)n2)cc1 ; c1ccc(-c2nc(-c3ccc4oc5ccccc5c4c3)nc(-c3ccc(-c4ccc5oc6ccccc6c5c4)c4oc5ccccc5c34)n2)cc1 ; c1ccc(-c2nc(-c3ccc4c(c3)oc3ccccc34)nc(-c3ccc(-c4ccc5oc6ccccc6c5c4)c4oc5ccccc5c34)n2)cc1]

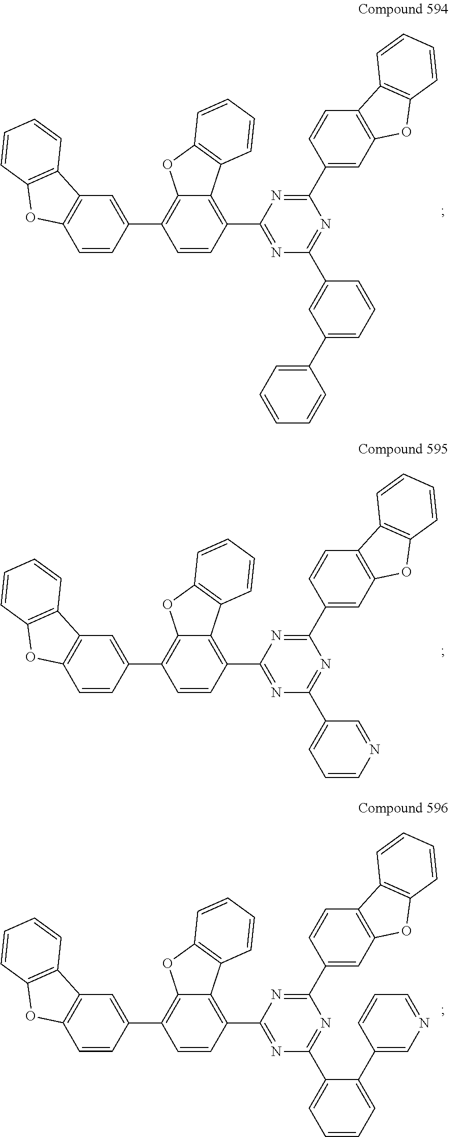 [SMILES: c1ccc(-c2cccc(-c3nc(-c4ccc5c(c4)oc4ccccc45)nc(-c4ccc(-c5ccc6oc7ccccc7c6c5)c5oc6ccccc6c45)n3)c2)cc1 ; c1cncc(-c2nc(-c3ccc4c(c3)oc3ccccc34)nc(-c3ccc(-c4ccc5oc6ccccc6c5c4)c4oc5ccccc5c34)n2)c1 ; c1cncc(-c2ccccc2-c2nc(-c3ccc4c(c3)oc3ccccc34)nc(-c3ccc(-c4ccc5oc6ccccc6c5c4)c4oc5ccccc5c34)n2)c1]

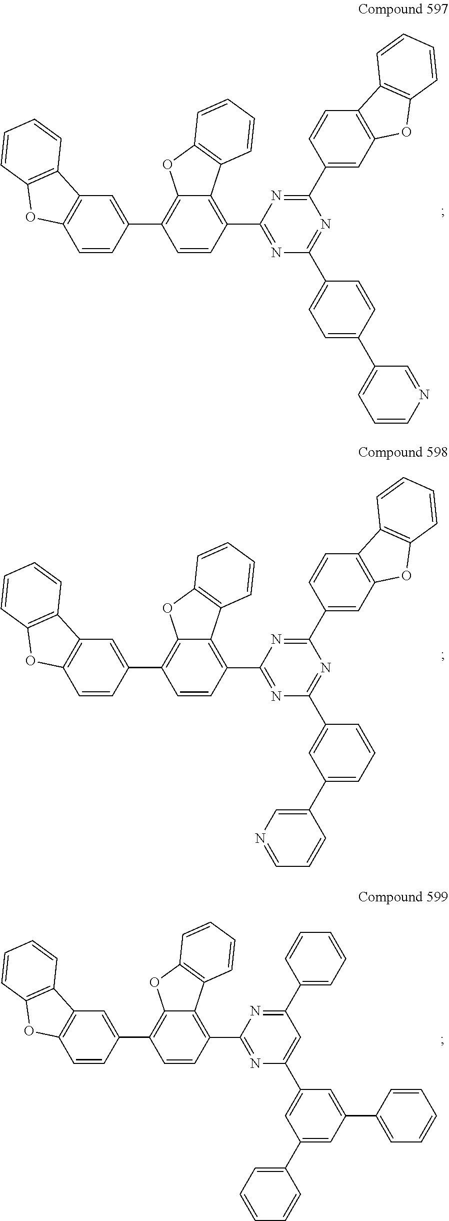 [SMILES: c1cncc(-c2ccc(-c3nc(-c4ccc5c(c4)oc4ccccc45)nc(-c4ccc(-c5ccc6oc7ccccc7c6c5)c5oc6ccccc6c45)n3)cc2)c1 ; c1cncc(-c2cccc(-c3nc(-c4ccc5c(c4)oc4ccccc45)nc(-c4ccc(-c5ccc6oc7ccccc7c6c5)c5oc6ccccc6c45)n3)c2)c1 ; c1ccc(-c2cc(-c3ccccc3)cc(-c3cc(-c4ccccc4)nc(-c4ccc(-c5ccc6oc7ccccc7c6c5)c5oc6ccccc6c45)n3)c2)cc1]

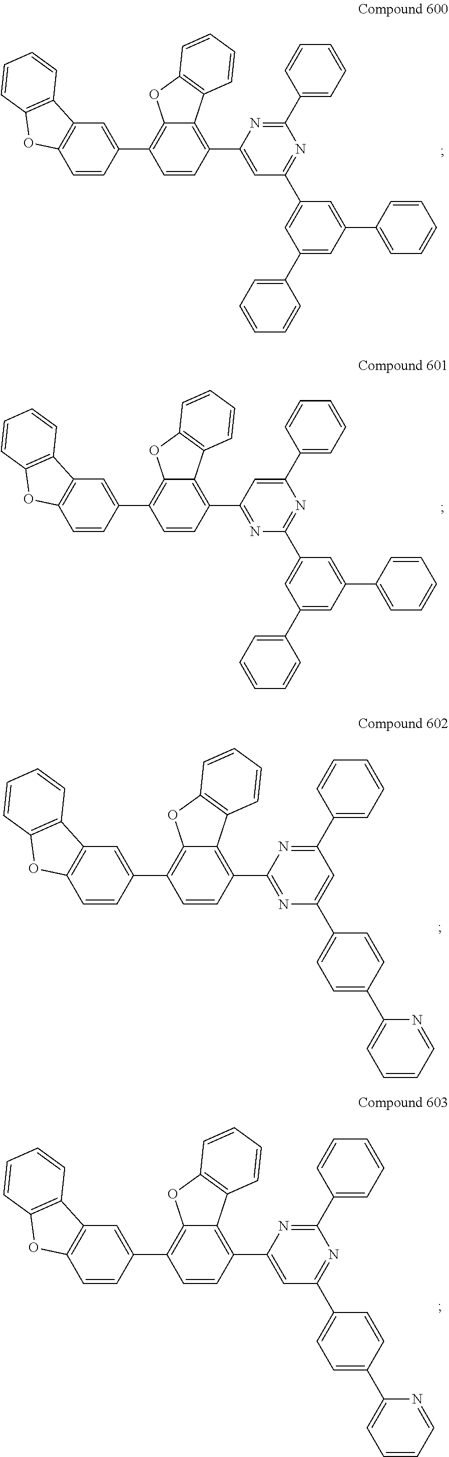 [SMILES: c1ccc(-c2cc(-c3ccccc3)cc(-c3cc(-c4ccc(-c5ccc6oc7ccccc7c6c5)c5oc6ccccc6c45)nc(-c4ccccc4)n3)c2)cc1 ; c1ccc(-c2cc(-c3ccccc3)cc(-c3nc(-c4ccccc4)cc(-c4ccc(-c5ccc6oc7ccccc7c6c5)c5oc6ccccc6c45)n3)c2)cc1 ; c1ccc(-c2cc(-c3ccc(-c4ccccn4)cc3)nc(-c3ccc(-c4ccc5oc6ccccc6c5c4)c4oc5ccccc5c34)n2)cc1 ; c1ccc(-c2nc(-c3ccc(-c4ccccn4)cc3)cc(-c3ccc(-c4ccc5oc6ccccc6c5c4)c4oc5ccccc5c34)n2)cc1]

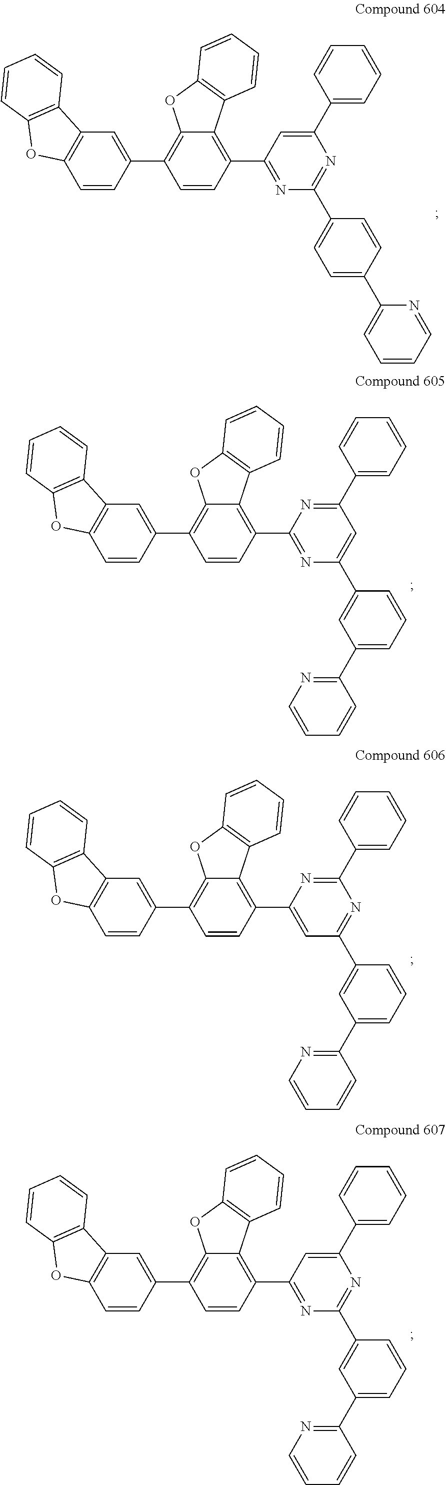 [SMILES: c1ccc(-c2cc(-c3ccc(-c4ccc5oc6ccccc6c5c4)c4oc5ccccc5c34)nc(-c3ccc(-c4ccccn4)cc3)n2)cc1 ; c1ccc(-c2cc(-c3cccc(-c4ccccn4)c3)nc(-c3ccc(-c4ccc5oc6ccccc6c5c4)c4oc5ccccc5c34)n2)cc1 ; c1ccc(-c2nc(-c3cccc(-c4ccccn4)c3)cc(-c3ccc(-c4ccc5oc6ccccc6c5c4)c4oc5ccccc5c34)n2)cc1 ; c1ccc(-c2cc(-c3ccc(-c4ccc5oc6ccccc6c5c4)c4oc5ccccc5c34)nc(-c3cccc(-c4ccccn4)c3)n2)cc1]

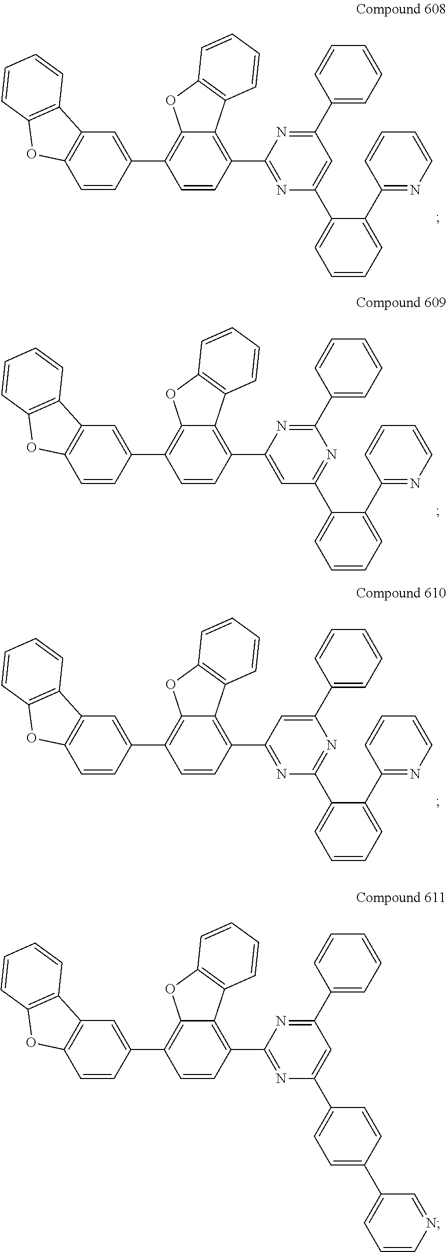 [SMILES: c1ccc(-c2cc(-c3ccccc3-c3ccccn3)nc(-c3ccc(-c4ccc5oc6ccccc6c5c4)c4oc5ccccc5c34)n2)cc1 ; c1ccc(-c2nc(-c3ccccc3-c3ccccn3)cc(-c3ccc(-c4ccc5oc6ccccc6c5c4)c4oc5ccccc5c34)n2)cc1 ; c1ccc(-c2cc(-c3ccc(-c4ccc5oc6ccccc6c5c4)c4oc5ccccc5c34)nc(-c3ccccc3-c3ccccn3)n2)cc1 ; c1ccc(-c2cc(-c3ccc(-c4cccnc4)cc3)nc(-c3ccc(-c4ccc5oc6ccccc6c5c4)c4oc5ccccc5c34)n2)cc1]

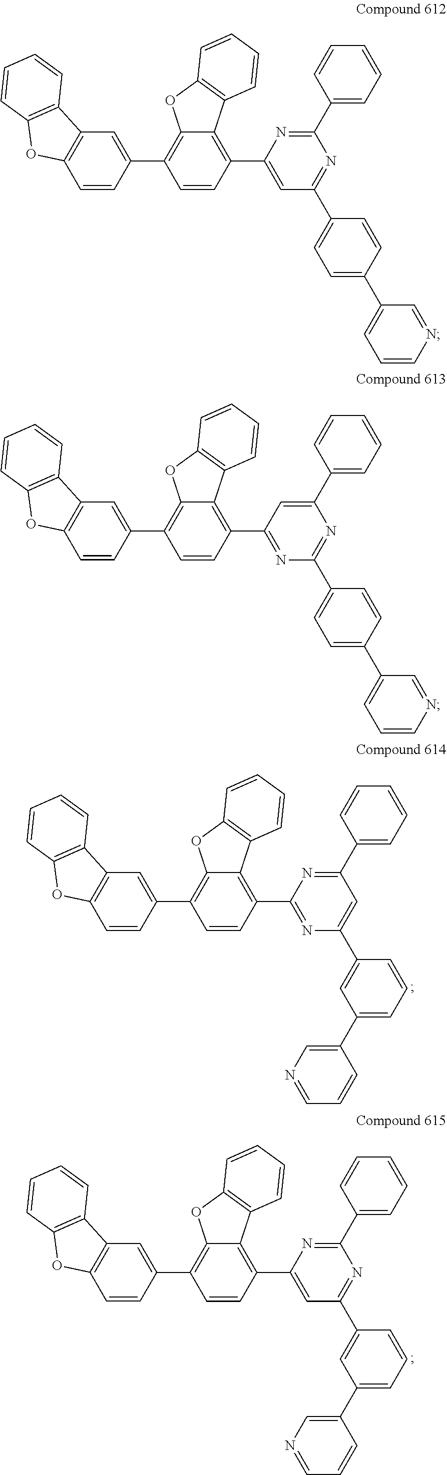 [SMILES: c1ccc(-c2nc(-c3ccc(-c4cccnc4)cc3)cc(-c3ccc(-c4ccc5oc6ccccc6c5c4)c4oc5ccccc5c34)n2)cc1 ; c1ccc(-c2cc(-c3ccc(-c4ccc5oc6ccccc6c5c4)c4oc5ccccc5c34)nc(-c3ccc(-c4cccnc4)cc3)n2)cc1 ; c1ccc(-c2cc(-c3cccc(-c4cccnc4)c3)nc(-c3ccc(-c4ccc5oc6ccccc6c5c4)c4oc5ccccc5c34)n2)cc1 ; c1ccc(-c2nc(-c3cccc(-c4cccnc4)c3)cc(-c3ccc(-c4ccc5oc6ccccc6c5c4)c4oc5ccccc5c34)n2)cc1]

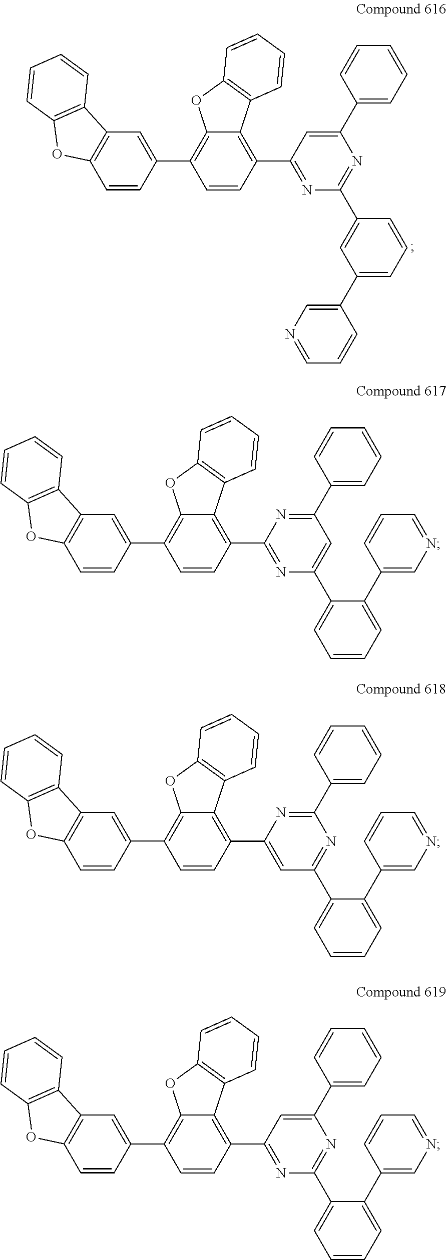 [SMILES: c1ccc(-c2cc(-c3ccc(-c4ccc5oc6ccccc6c5c4)c4oc5ccccc5c34)nc(-c3cccc(-c4cccnc4)c3)n2)cc1 ; c1ccc(-c2cc(-c3ccccc3-c3cccnc3)nc(-c3ccc(-c4ccc5oc6ccccc6c5c4)c4oc5ccccc5c34)n2)cc1 ; c1ccc(-c2nc(-c3ccccc3-c3cccnc3)cc(-c3ccc(-c4ccc5oc6ccccc6c5c4)c4oc5ccccc5c34)n2)cc1 ; c1ccc(-c2cc(-c3ccc(-c4ccc5oc6ccccc6c5c4)c4oc5ccccc5c34)nc(-c3ccccc3-c3cccnc3)n2)cc1]

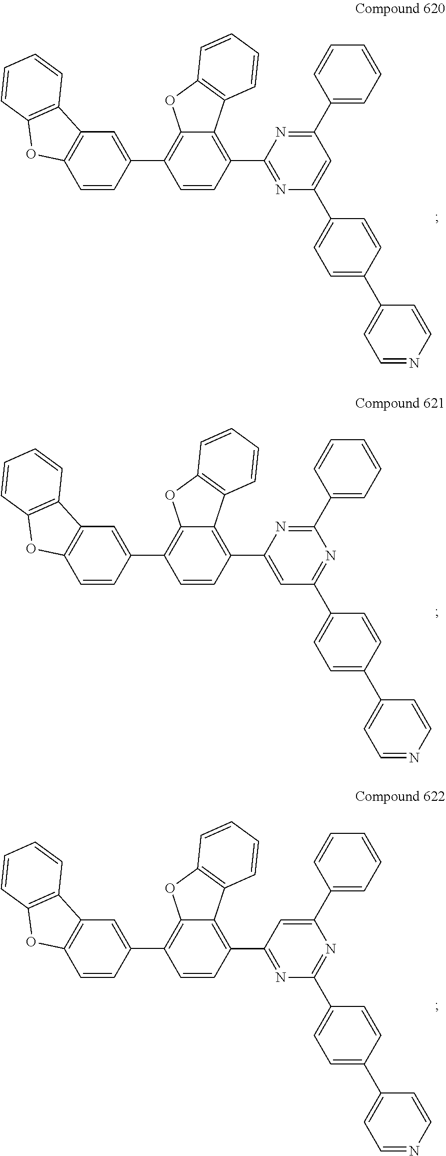 [SMILES: c1ccc(-c2cc(-c3ccc(-c4ccncc4)cc3)nc(-c3ccc(-c4ccc5oc6ccccc6c5c4)c4oc5ccccc5c34)n2)cc1 ; c1ccc(-c2nc(-c3ccc(-c4ccncc4)cc3)cc(-c3ccc(-c4ccc5oc6ccccc6c5c4)c4oc5ccccc5c34)n2)cc1 ; c1ccc(-c2cc(-c3ccc(-c4ccc5oc6ccccc6c5c4)c4oc5ccccc5c34)nc(-c3ccc(-c4ccncc4)cc3)n2)cc1]

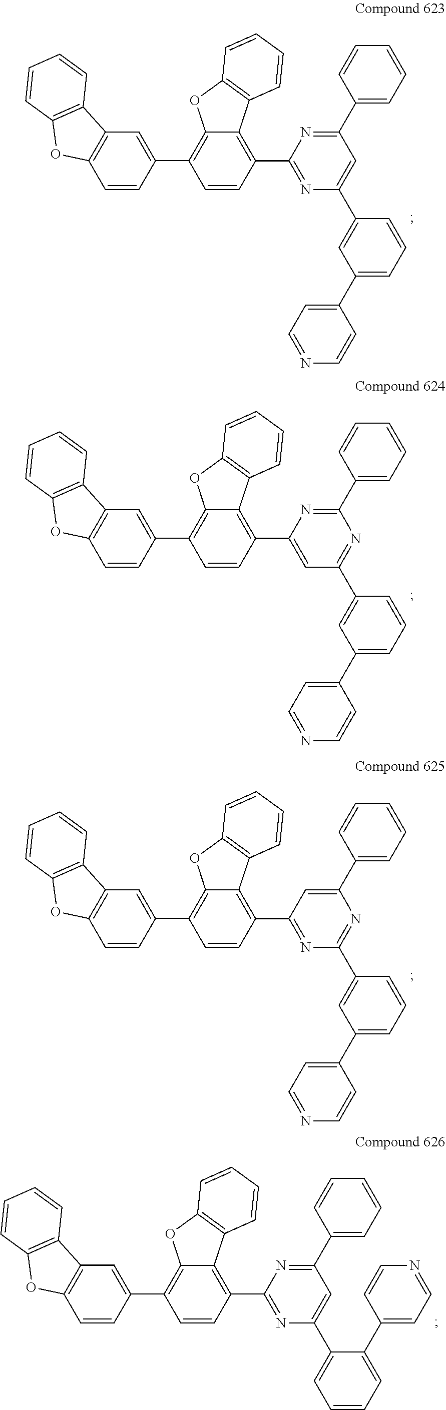 [SMILES: c1ccc(-c2cc(-c3cccc(-c4ccncc4)c3)nc(-c3ccc(-c4ccc5oc6ccccc6c5c4)c4oc5ccccc5c34)n2)cc1 ; c1ccc(-c2nc(-c3cccc(-c4ccncc4)c3)cc(-c3ccc(-c4ccc5oc6ccccc6c5c4)c4oc5ccccc5c34)n2)cc1 ; c1ccc(-c2cc(-c3ccc(-c4ccc5oc6ccccc6c5c4)c4oc5ccccc5c34)nc(-c3cccc(-c4ccncc4)c3)n2)cc1 ; c1ccc(-c2cc(-c3ccccc3-c3ccncc3)nc(-c3ccc(-c4ccc5oc6ccccc6c5c4)c4oc5ccccc5c34)n2)cc1]

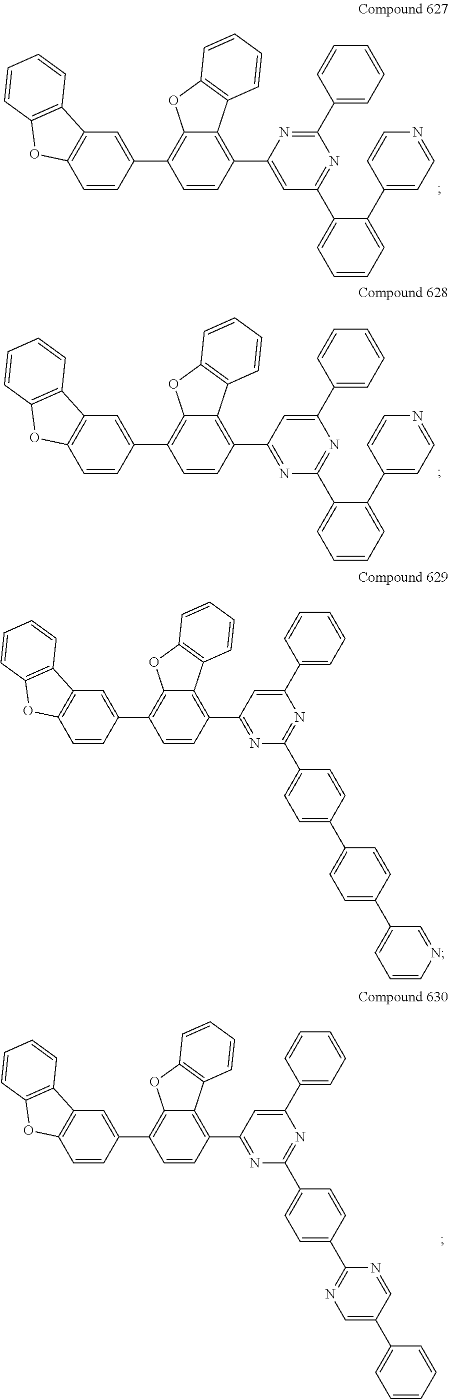 [SMILES: c1ccc(-c2nc(-c3ccccc3-c3ccncc3)cc(-c3ccc(-c4ccc5oc6ccccc6c5c4)c4oc5ccccc5c34)n2)cc1 ; c1ccc(-c2cc(-c3ccc(-c4ccc5oc6ccccc6c5c4)c4oc5ccccc5c34)nc(-c3ccccc3-c3ccncc3)n2)cc1 ; c1ccc(-c2cc(-c3ccc(-c4ccc5oc6ccccc6c5c4)c4oc5ccccc5c34)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc7oc8ccccc8c7c6)c6oc7ccccc7c56)n4)cc3)nc2)cc1]

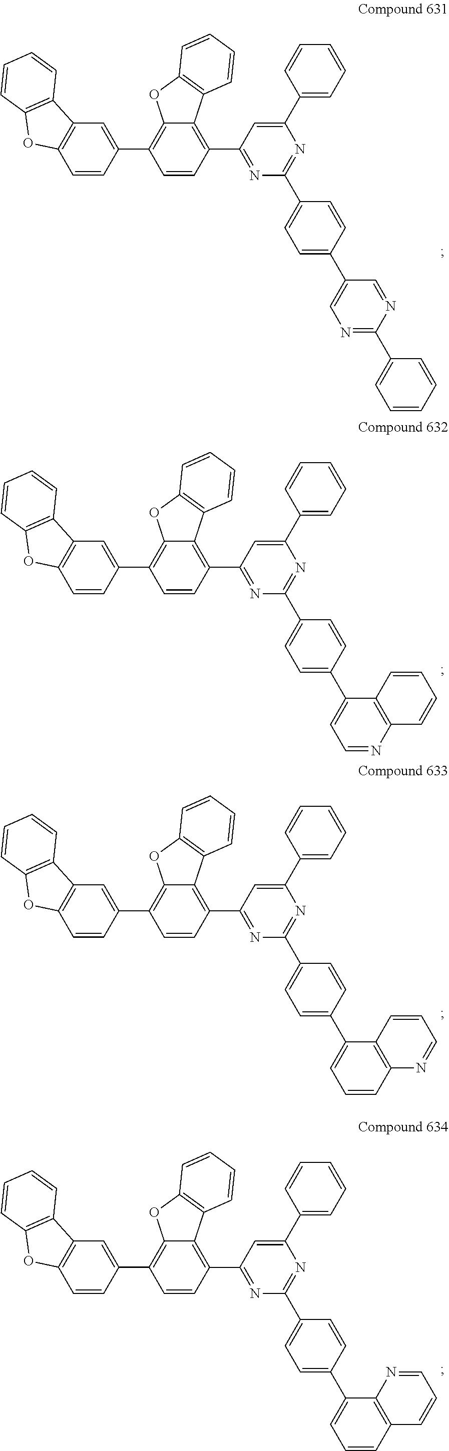 [SMILES: c1ccc(-c2cc(-c3ccc(-c4ccc5oc6ccccc6c5c4)c4oc5ccccc5c34)nc(-c3ccc(-c4cnc(-c5ccccc5)nc4)cc3)n2)cc1 ; c1ccc(-c2cc(-c3ccc(-c4ccc5oc6ccccc6c5c4)c4oc5ccccc5c34)nc(-c3ccc(-c4ccnc5ccccc45)cc3)n2)cc1 ; c1ccc(-c2cc(-c3ccc(-c4ccc5oc6ccccc6c5c4)c4oc5ccccc5c34)nc(-c3ccc(-c4cccc5ncccc45)cc3)n2)cc1 ; c1ccc(-c2cc(-c3ccc(-c4ccc5oc6ccccc6c5c4)c4oc5ccccc5c34)nc(-c3ccc(-c4cccc5cccnc45)cc3)n2)cc1]

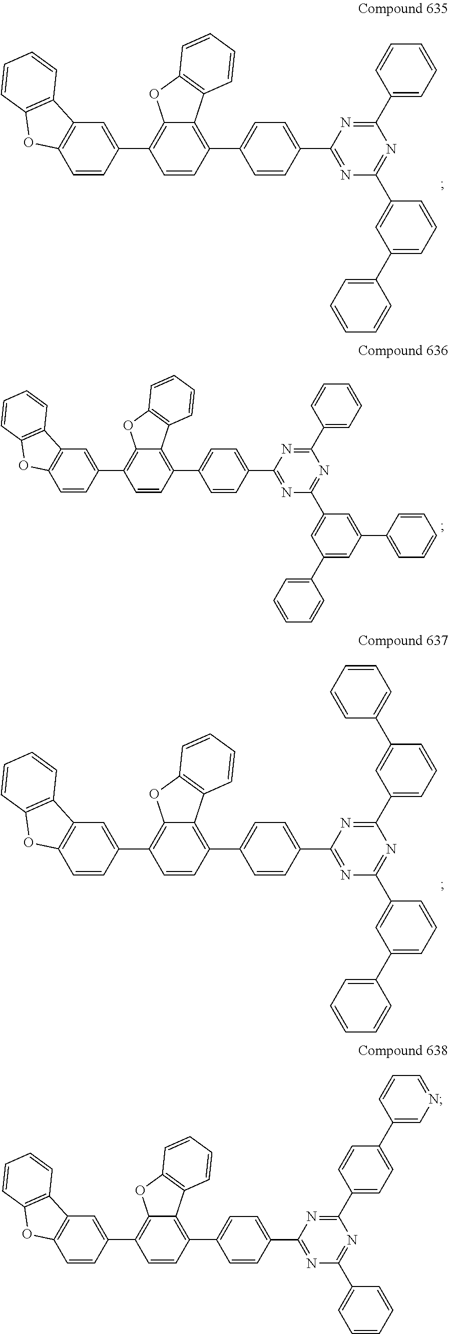 [SMILES: c1ccc(-c2cccc(-c3nc(-c4ccccc4)nc(-c4ccc(-c5ccc(-c6ccc7oc8ccccc8c7c6)c6oc7ccccc7c56)cc4)n3)c2)cc1 ; c1ccc(-c2cc(-c3ccccc3)cc(-c3nc(-c4ccccc4)nc(-c4ccc(-c5ccc(-c6ccc7oc8ccccc8c7c6)c6oc7ccccc7c56)cc4)n3)c2)cc1 ; c1ccc(-c2cccc(-c3nc(-c4ccc(-c5ccc(-c6ccc7oc8ccccc8c7c6)c6oc7ccccc7c56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5ccc6oc7ccccc7c6c5)c5oc6ccccc6c45)cc3)n2)cc1]

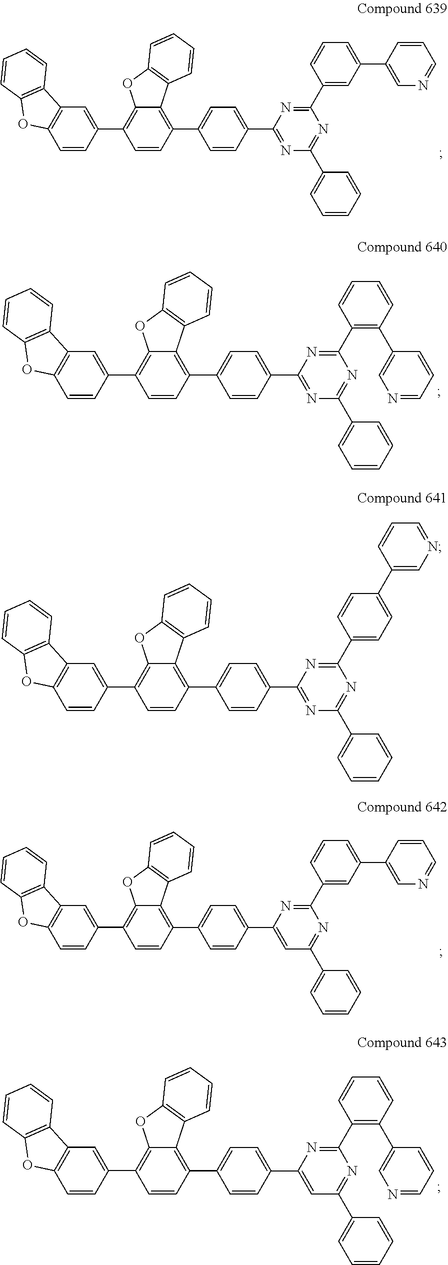 [SMILES: c1ccc(-c2nc(-c3ccc(-c4ccc(-c5ccc6oc7ccccc7c6c5)c5oc6ccccc6c45)cc3)nc(-c3cccc(-c4cccnc4)c3)n2)cc1 ; c1ccc(-c2nc(-c3ccc(-c4ccc(-c5ccc6oc7ccccc7c6c5)c5oc6ccccc6c45)cc3)nc(-c3ccccc3-c3cccnc3)n2)cc1 ; c1ccc(-c2nc(-c3ccc(-c4cccnc4)cc3)nc(-c3ccc(-c4ccc(-c5ccc6oc7ccccc7c6c5)c5oc6ccccc6c45)cc3)n2)cc1 ; c1ccc(-c2cc(-c3ccc(-c4ccc(-c5ccc6oc7ccccc7c6c5)c5oc6ccccc6c45)cc3)nc(-c3cccc(-c4cccnc4)c3)n2)cc1 ; c1ccc(-c2cc(-c3ccc(-c4ccc(-c5ccc6oc7ccccc7c6c5)c5oc6ccccc6c45)cc3)nc(-c3ccccc3-c3cccnc3)n2)cc1]

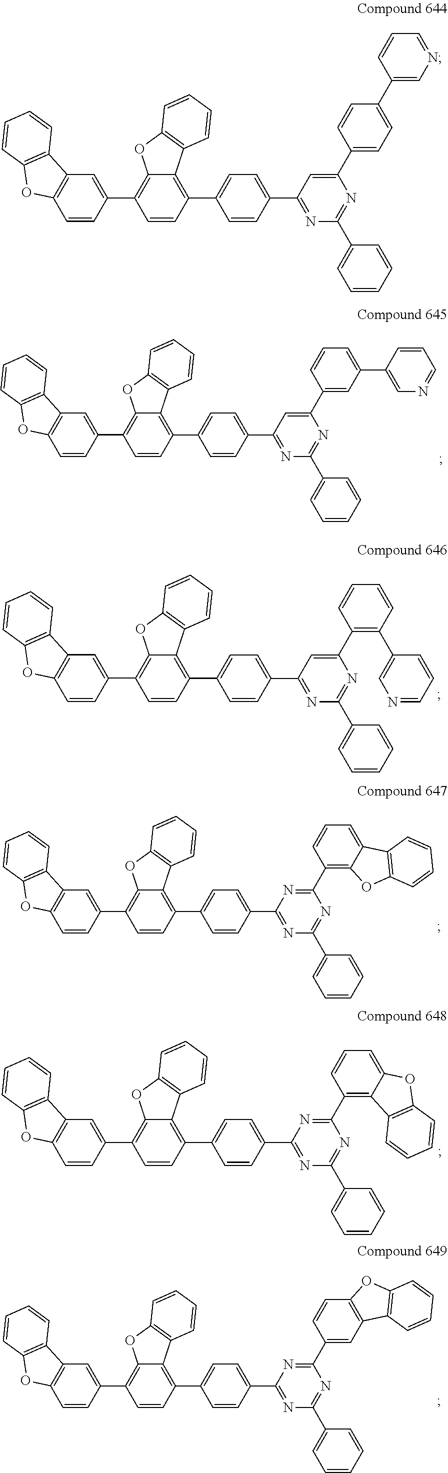

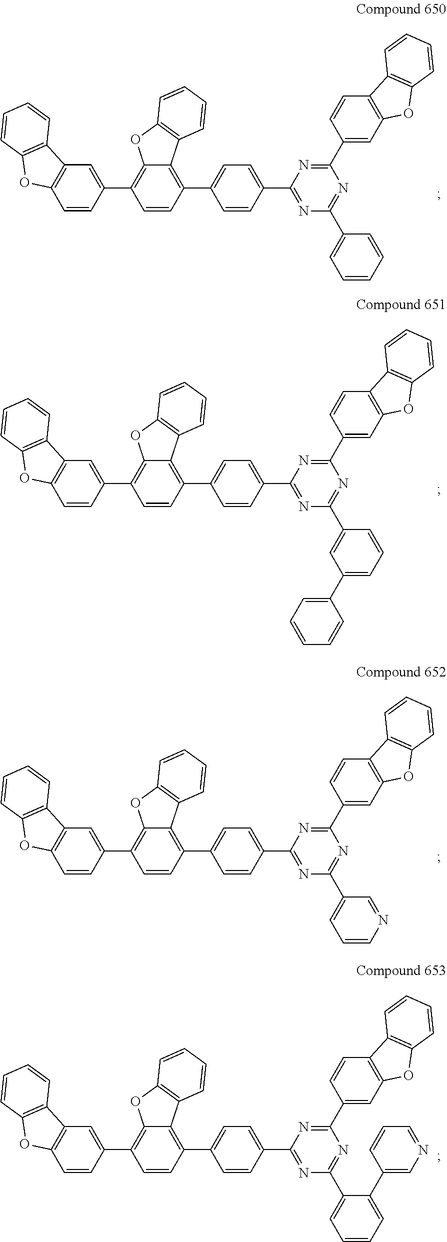 [SMILES: c1ccc(-c2nc(-c3ccc(-c4ccc(-c5ccc6oc7ccccc7c6c5)c5oc6ccccc6c45)cc3)nc(-c3ccc4c(c3)oc3ccccc34)n2)cc1 ; c1ccc(-c2cccc(-c3nc(-c4ccc(-c5ccc(-c6ccc7oc8ccccc8c7c6)c6oc7ccccc7c56)cc4)nc(-c4ccc5c(c4)oc4ccccc45)n3)c2)cc1 ; c1cncc(-c2nc(-c3ccc(-c4ccc(-c5ccc6oc7ccccc7c6c5)c5oc6ccccc6c45)cc3)nc(-c3ccc4c(c3)oc3ccccc34)n2)c1 ; c1cncc(-c2ccccc2-c2nc(-c3ccc(-c4ccc(-c5ccc6oc7ccccc7c6c5)c5oc6ccccc6c45)cc3)nc(-c3ccc4c(c3)oc3ccccc34)n2)c1]

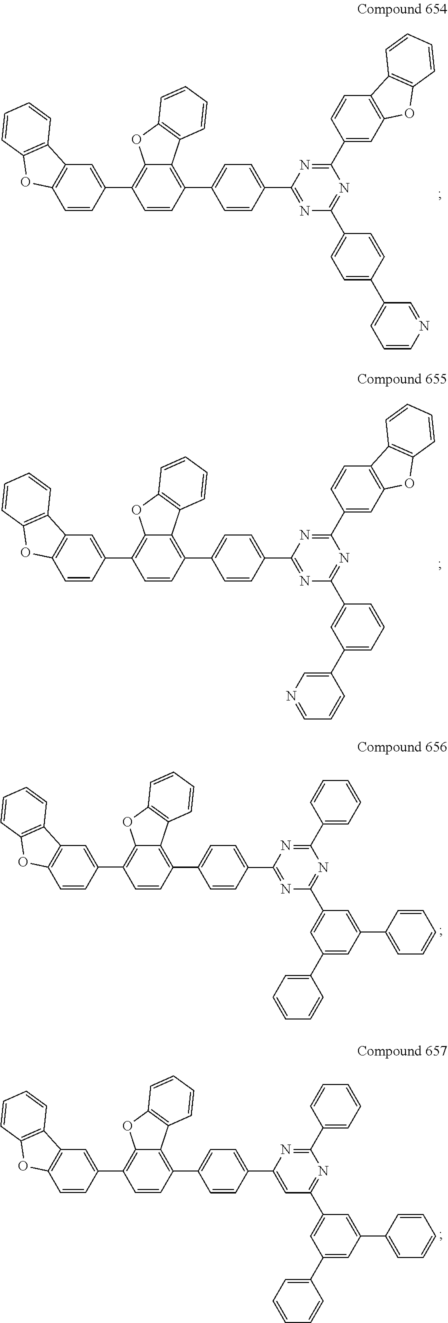 [SMILES: c1cncc(-c2ccc(-c3nc(-c4ccc(-c5ccc(-c6ccc7oc8ccccc8c7c6)c6oc7ccccc7c56)cc4)nc(-c4ccc5c(c4)oc4ccccc45)n3)cc2)c1 ; c1cncc(-c2cccc(-c3nc(-c4ccc(-c5ccc(-c6ccc7oc8ccccc8c7c6)c6oc7ccccc7c56)cc4)nc(-c4ccc5c(c4)oc4ccccc45)n3)c2)c1 ; c1ccc(-c2cc(-c3ccccc3)cc(-c3nc(-c4ccccc4)nc(-c4ccc(-c5ccc(-c6ccc7oc8ccccc8c7c6)c6oc7ccccc7c56)cc4)n3)c2)cc1 ; c1ccc(-c2cc(-c3ccccc3)cc(-c3cc(-c4ccc(-c5ccc(-c6ccc7oc8ccccc8c7c6)c6oc7ccccc7c56)cc4)nc(-c4ccccc4)n3)c2)cc1]

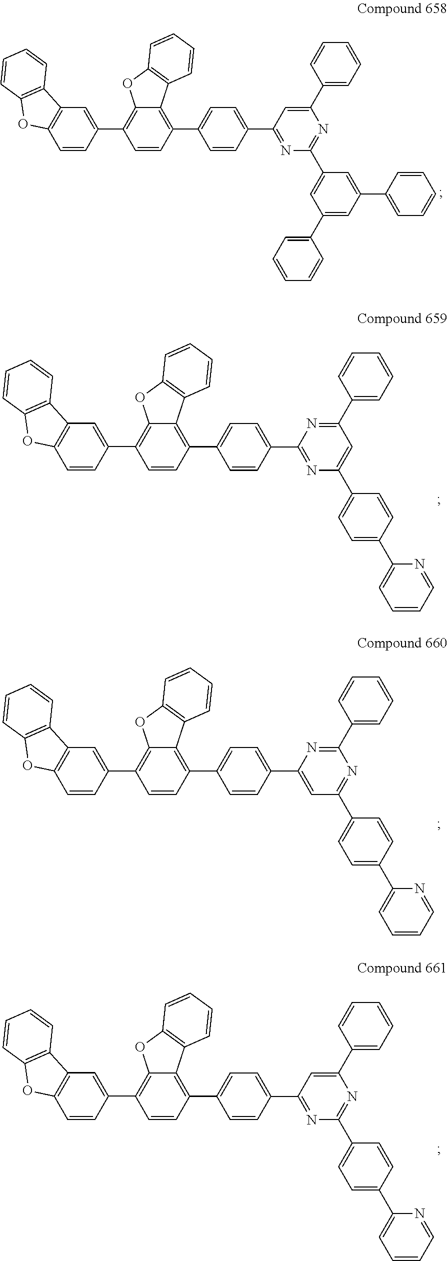 [SMILES: c1ccc(-c2cc(-c3ccccc3)cc(-c3nc(-c4ccccc4)cc(-c4ccc(-c5ccc(-c6ccc7oc8ccccc8c7c6)c6oc7ccccc7c56)cc4)n3)c2)cc1 ; c1ccc(-c2cc(-c3ccc(-c4ccccn4)cc3)nc(-c3ccc(-c4ccc(-c5ccc6oc7ccccc7c6c5)c5oc6ccccc6c45)cc3)n2)cc1 ; c1ccc(-c2nc(-c3ccc(-c4ccccn4)cc3)cc(-c3ccc(-c4ccc(-c5ccc6oc7ccccc7c6c5)c5oc6ccccc6c45)cc3)n2)cc1 ; c1ccc(-c2cc(-c3ccc(-c4ccc(-c5ccc6oc7ccccc7c6c5)c5oc6ccccc6c45)cc3)nc(-c3ccc(-c4ccccn4)cc3)n2)cc1]

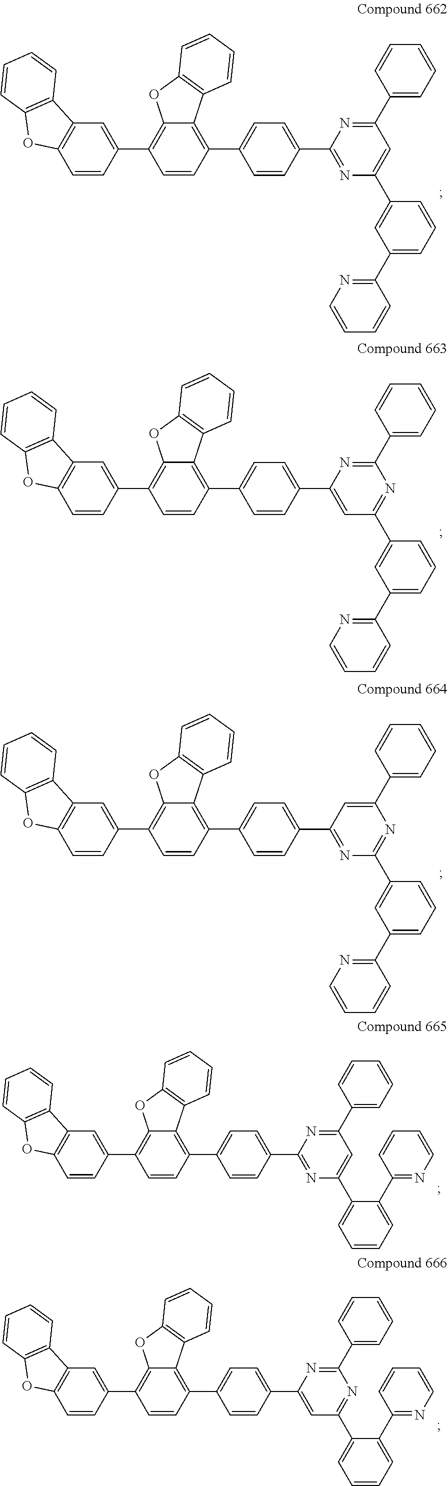 [SMILES: c1ccc(-c2cc(-c3cccc(-c4ccccn4)c3)nc(-c3ccc(-c4ccc(-c5ccc6oc7ccccc7c6c5)c5oc6ccccc6c45)cc3)n2)cc1 ; c1ccc(-c2nc(-c3ccc(-c4ccc(-c5ccc6oc7ccccc7c6c5)c5oc6ccccc6c45)cc3)cc(-c3cccc(-c4ccccn4)c3)n2)cc1 ; c1ccc(-c2cc(-c3ccc(-c4ccc(-c5ccc6oc7ccccc7c6c5)c5oc6ccccc6c45)cc3)nc(-c3cccc(-c4ccccn4)c3)n2)cc1 ; c1ccc(-c2cc(-c3ccccc3-c3ccccn3)nc(-c3ccc(-c4ccc(-c5ccc6oc7ccccc7c6c5)c5oc6ccccc6c45)cc3)n2)cc1 ; c1ccc(-c2nc(-c3ccc(-c4ccc(-c5ccc6oc7ccccc7c6c5)c5oc6ccccc6c45)cc3)cc(-c3ccccc3-c3ccccn3)n2)cc1]

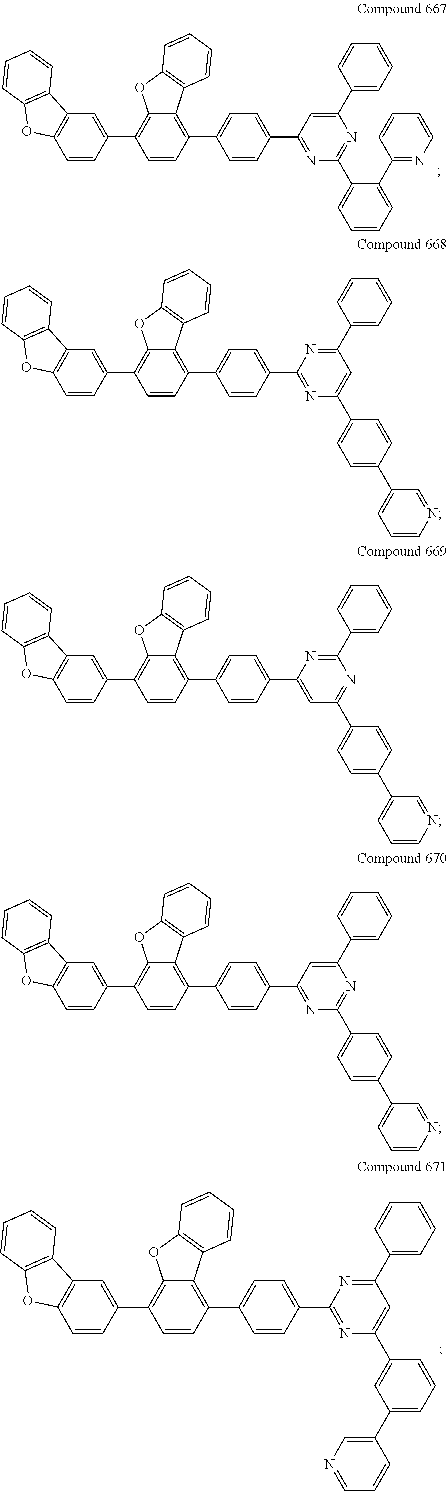 [SMILES: c1ccc(-c2cc(-c3ccc(-c4ccc(-c5ccc6oc7ccccc7c6c5)c5oc6ccccc6c45)cc3)nc(-c3ccccc3-c3ccccn3)n2)cc1 ; c1ccc(-c2cc(-c3ccc(-c4cccnc4)cc3)nc(-c3ccc(-c4ccc(-c5ccc6oc7ccccc7c6c5)c5oc6ccccc6c45)cc3)n2)cc1 ; c1ccc(-c2nc(-c3ccc(-c4cccnc4)cc3)cc(-c3ccc(-c4ccc(-c5ccc6oc7ccccc7c6c5)c5oc6ccccc6c45)cc3)n2)cc1 ; c1ccc(-c2cc(-c3ccc(-c4ccc(-c5ccc6oc7ccccc7c6c5)c5oc6ccccc6c45)cc3)nc(-c3ccc(-c4cccnc4)cc3)n2)cc1 ; c1ccc(-c2cc(-c3cccc(-c4cccnc4)c3)nc(-c3ccc(-c4ccc(-c5ccc6oc7ccccc7c6c5)c5oc6ccccc6c45)cc3)n2)cc1]

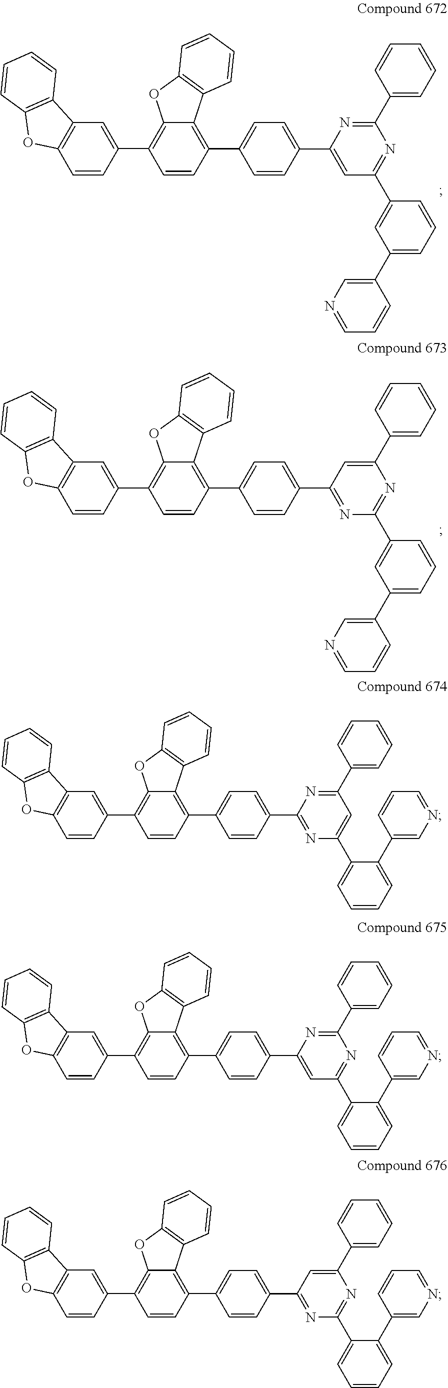 [SMILES: c1ccc(-c2nc(-c3ccc(-c4ccc(-c5ccc6oc7ccccc7c6c5)c5oc6ccccc6c45)cc3)cc(-c3cccc(-c4cccnc4)c3)n2)cc1 ; c1ccc(-c2cc(-c3ccc(-c4ccc(-c5ccc6oc7ccccc7c6c5)c5oc6ccccc6c45)cc3)nc(-c3cccc(-c4cccnc4)c3)n2)cc1 ; c1ccc(-c2cc(-c3ccccc3-c3cccnc3)nc(-c3ccc(-c4ccc(-c5ccc6oc7ccccc7c6c5)c5oc6ccccc6c45)cc3)n2)cc1 ; c1ccc(-c2nc(-c3ccc(-c4ccc(-c5ccc6oc7ccccc7c6c5)c5oc6ccccc6c45)cc3)cc(-c3ccccc3-c3cccnc3)n2)cc1 ; c1ccc(-c2cc(-c3ccc(-c4ccc(-c5ccc6oc7ccccc7c6c5)c5oc6ccccc6c45)cc3)nc(-c3ccccc3-c3cccnc3)n2)cc1]

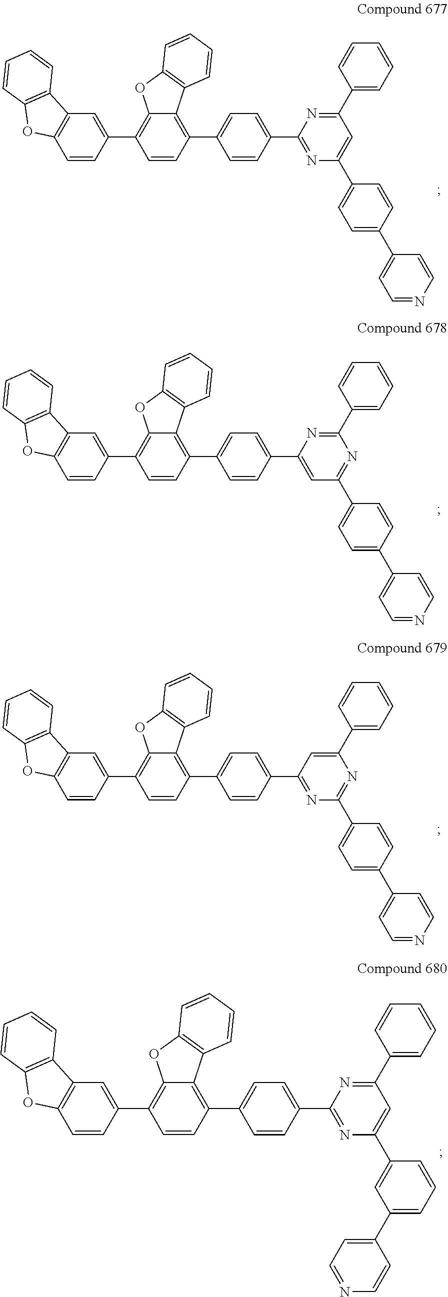 [SMILES: c1ccc(-c2cc(-c3ccc(-c4ccncc4)cc3)nc(-c3ccc(-c4ccc(-c5ccc6oc7ccccc7c6c5)c5oc6ccccc6c45)cc3)n2)cc1 ; c1ccc(-c2nc(-c3ccc(-c4ccncc4)cc3)cc(-c3ccc(-c4ccc(-c5ccc6oc7ccccc7c6c5)c5oc6ccccc6c45)cc3)n2)cc1 ; c1ccc(-c2cc(-c3ccc(-c4ccc(-c5ccc6oc7ccccc7c6c5)c5oc6ccccc6c45)cc3)nc(-c3ccc(-c4ccncc4)cc3)n2)cc1 ; c1ccc(-c2cc(-c3cccc(-c4ccncc4)c3)nc(-c3ccc(-c4ccc(-c5ccc6oc7ccccc7c6c5)c5oc6ccccc6c45)cc3)n2)cc1]

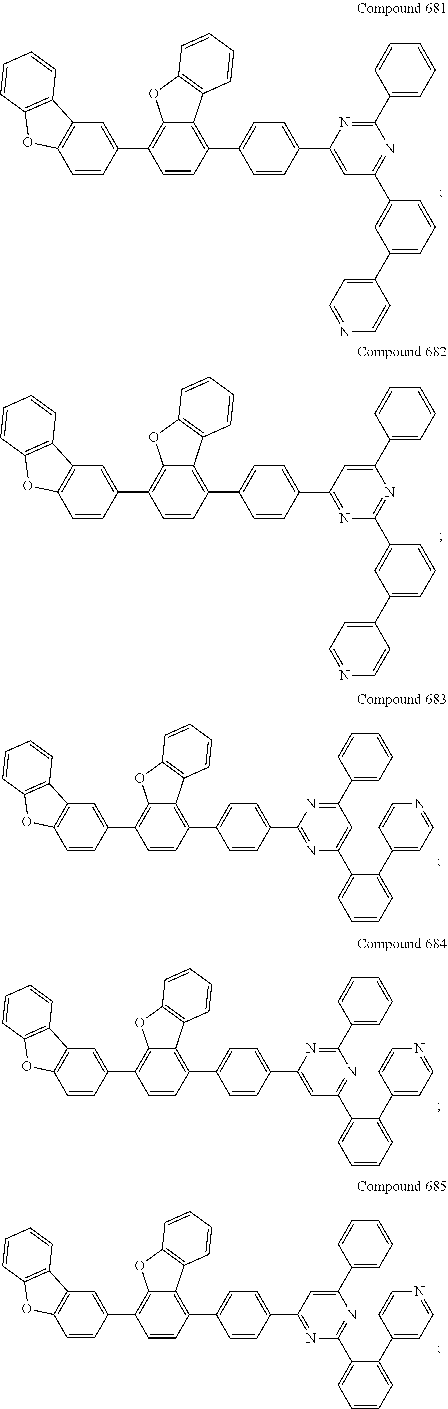 [SMILES: c1ccc(-c2nc(-c3ccc(-c4ccc(-c5ccc6oc7ccccc7c6c5)c5oc6ccccc6c45)cc3)cc(-c3cccc(-c4ccncc4)c3)n2)cc1 ; c1ccc(-c2cc(-c3ccc(-c4ccc(-c5ccc6oc7ccccc7c6c5)c5oc6ccccc6c45)cc3)nc(-c3cccc(-c4ccncc4)c3)n2)cc1 ; c1ccc(-c2cc(-c3ccccc3-c3ccncc3)nc(-c3ccc(-c4ccc(-c5ccc6oc7ccccc7c6c5)c5oc6ccccc6c45)cc3)n2)cc1 ; c1ccc(-c2nc(-c3ccc(-c4ccc(-c5ccc6oc7ccccc7c6c5)c5oc6ccccc6c45)cc3)cc(-c3ccccc3-c3ccncc3)n2)cc1 ; c1ccc(-c2cc(-c3ccc(-c4ccc(-c5ccc6oc7ccccc7c6c5)c5oc6ccccc6c45)cc3)nc(-c3ccccc3-c3ccncc3)n2)cc1]

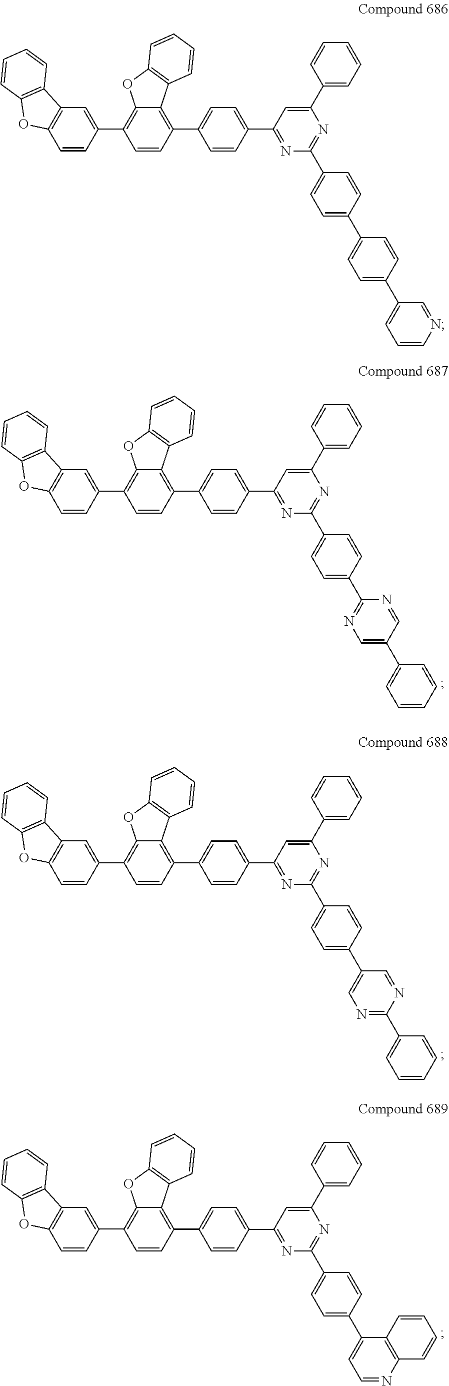 [SMILES: c1ccc(-c2cc(-c3ccc(-c4ccc(-c5ccc6oc7ccccc7c6c5)c5oc6ccccc6c45)cc3)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc(-c7ccc8oc9ccccc9c8c7)c7oc8ccccc8c67)cc5)n4)cc3)nc2)cc1 ; c1ccc(-c2cc(-c3ccc(-c4ccc(-c5ccc6oc7ccccc7c6c5)c5oc6ccccc6c45)cc3)nc(-c3ccc(-c4cnc(-c5ccccc5)nc4)cc3)n2)cc1 ; c1ccc(-c2cc(-c3ccc(-c4ccc(-c5ccc6oc7ccccc7c6c5)c5oc6ccccc6c45)cc3)nc(-c3ccc(-c4ccnc5ccccc45)cc3)n2)cc1]

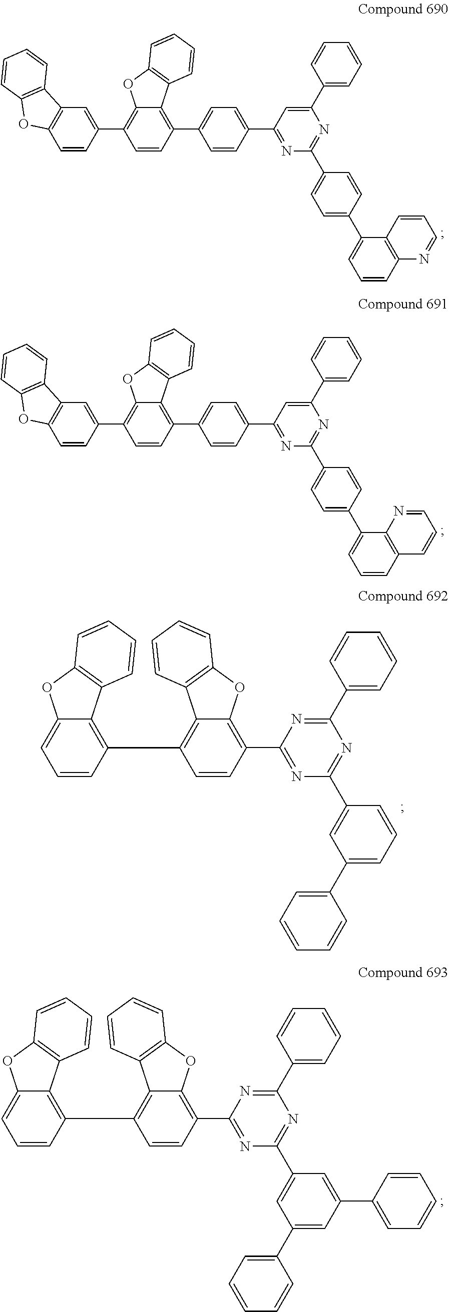 [SMILES: c1ccc(-c2cc(-c3ccc(-c4ccc(-c5ccc6oc7ccccc7c6c5)c5oc6ccccc6c45)cc3)nc(-c3ccc(-c4cccc5ncccc45)cc3)n2)cc1 ; c1ccc(-c2cc(-c3ccc(-c4ccc(-c5ccc6oc7ccccc7c6c5)c5oc6ccccc6c45)cc3)nc(-c3ccc(-c4cccc5cccnc45)cc3)n2)cc1 ; c1ccc(-c2cccc(-c3nc(-c4ccccc4)nc(-c4ccc(-c5cccc6oc7ccccc7c56)c5c4oc4ccccc45)n3)c2)cc1 ; c1ccc(-c2cc(-c3ccccc3)cc(-c3nc(-c4ccccc4)nc(-c4ccc(-c5cccc6oc7ccccc7c56)c5c4oc4ccccc45)n3)c2)cc1]

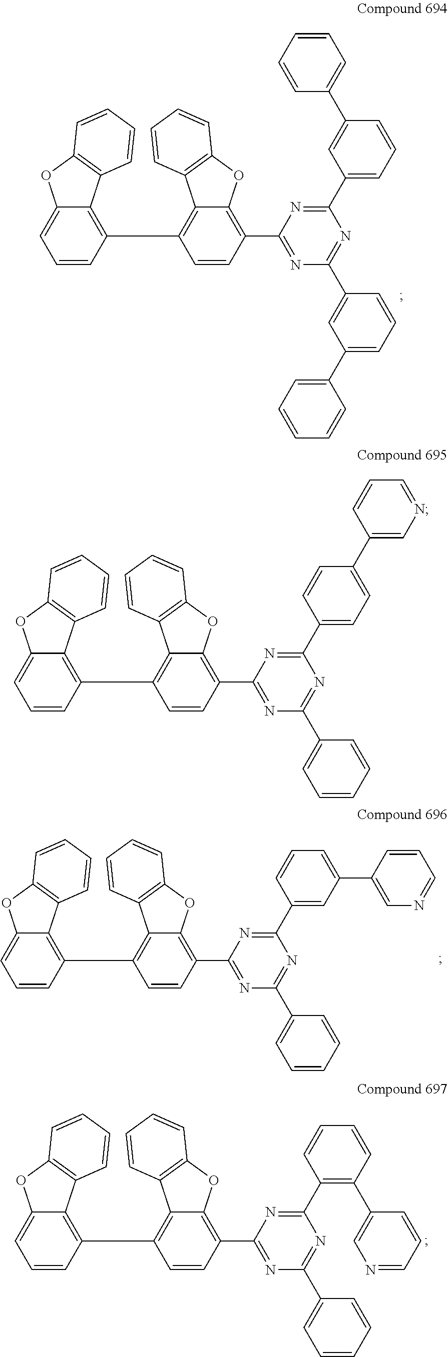 [SMILES: c1ccc(-c2cccc(-c3nc(-c4cccc(-c5ccccc5)c4)nc(-c4ccc(-c5cccc6oc7ccccc7c56)c5c4oc4ccccc45)n3)c2)cc1 ; c1ccc(-c2nc(-c3ccc(-c4cccnc4)cc3)nc(-c3ccc(-c4cccc5oc6ccccc6c45)c4c3oc3ccccc34)n2)cc1 ; c1ccc(-c2nc(-c3cccc(-c4cccnc4)c3)nc(-c3ccc(-c4cccc5oc6ccccc6c45)c4c3oc3ccccc34)n2)cc1 ; c1ccc(-c2nc(-c3ccccc3-c3cccnc3)nc(-c3ccc(-c4cccc5oc6ccccc6c45)c4c3oc3ccccc34)n2)cc1]

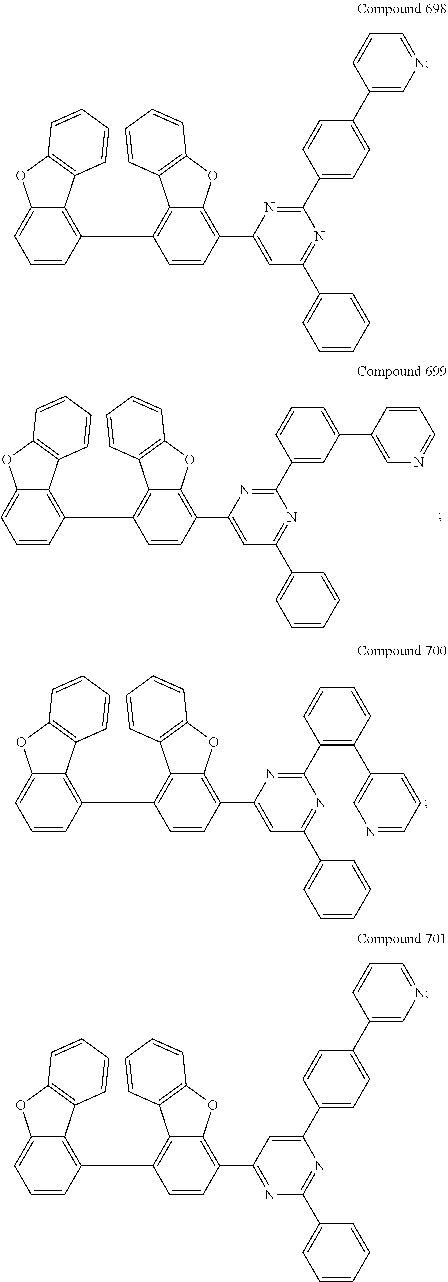 [SMILES: c1ccc(-c2cc(-c3ccc(-c4cccc5oc6ccccc6c45)c4c3oc3ccccc34)nc(-c3ccc(-c4cccnc4)cc3)n2)cc1 ; c1ccc(-c2cc(-c3ccc(-c4cccc5oc6ccccc6c45)c4c3oc3ccccc34)nc(-c3cccc(-c4cccnc4)c3)n2)cc1 ; c1ccc(-c2cc(-c3ccc(-c4cccc5oc6ccccc6c45)c4c3oc3ccccc34)nc(-c3ccccc3-c3cccnc3)n2)cc1 ; c1ccc(-c2nc(-c3ccc(-c4cccnc4)cc3)cc(-c3ccc(-c4cccc5oc6ccccc6c45)c4c3oc3ccccc34)n2)cc1]

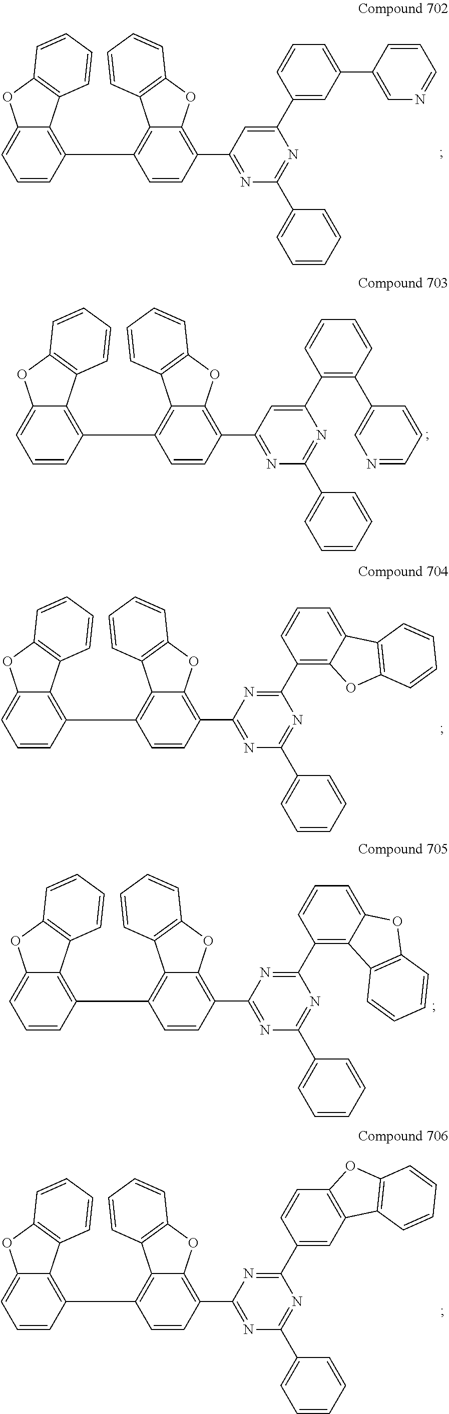 [SMILES: c1ccc(-c2nc(-c3cccc(-c4cccnc4)c3)cc(-c3ccc(-c4cccc5oc6ccccc6c45)c4c3oc3ccccc34)n2)cc1 ; c1ccc(-c2nc(-c3ccccc3-c3cccnc3)cc(-c3ccc(-c4cccc5oc6ccccc6c45)c4c3oc3ccccc34)n2)cc1 ; c1ccc(-c2nc(-c3cccc4c3oc3ccccc34)nc(-c3ccc(-c4cccc5oc6ccccc6c45)c4c3oc3ccccc34)n2)cc1 ; c1ccc(-c2nc(-c3ccc(-c4cccc5oc6ccccc6c45)c4c3oc3ccccc34)nc(-c3cccc4oc5ccccc5c34)n2)cc1 ; c1ccc(-c2nc(-c3ccc4oc5ccccc5c4c3)nc(-c3ccc(-c4cccc5oc6ccccc6c45)c4c3oc3ccccc34)n2)cc1]

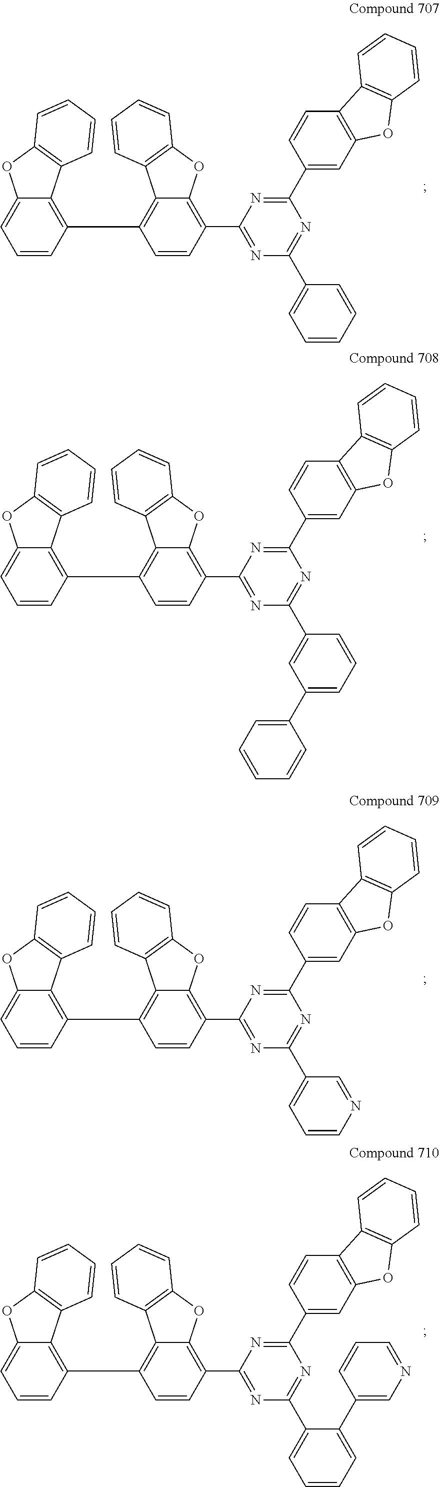 [SMILES: c1ccc(-c2nc(-c3ccc4c(c3)oc3ccccc34)nc(-c3ccc(-c4cccc5oc6ccccc6c45)c4c3oc3ccccc34)n2)cc1 ; c1ccc(-c2cccc(-c3nc(-c4ccc5c(c4)oc4ccccc45)nc(-c4ccc(-c5cccc6oc7ccccc7c56)c5c4oc4ccccc45)n3)c2)cc1 ; c1cncc(-c2nc(-c3ccc4c(c3)oc3ccccc34)nc(-c3ccc(-c4cccc5oc6ccccc6c45)c4c3oc3ccccc34)n2)c1 ; c1cncc(-c2ccccc2-c2nc(-c3ccc4c(c3)oc3ccccc34)nc(-c3ccc(-c4cccc5oc6ccccc6c45)c4c3oc3ccccc34)n2)c1]

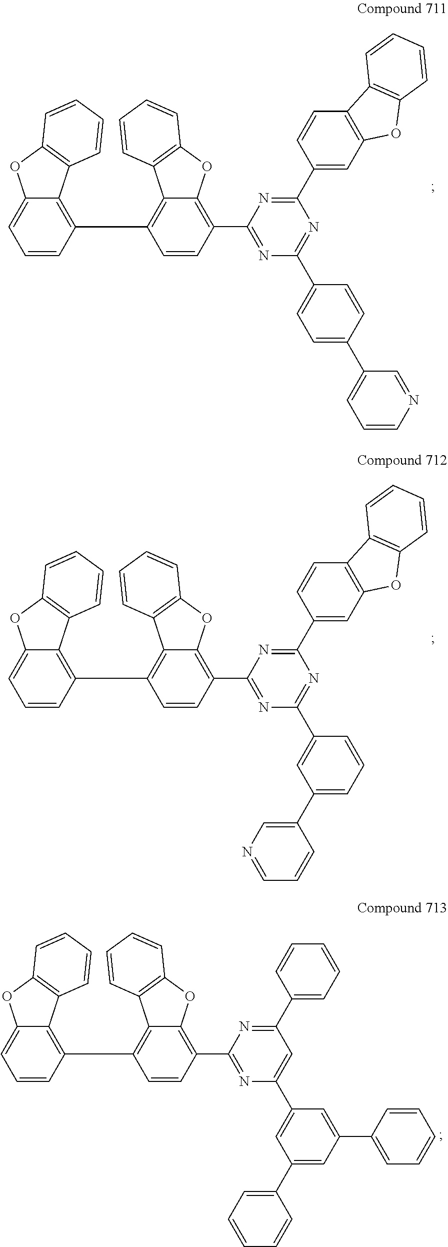 [SMILES: c1cncc(-c2ccc(-c3nc(-c4ccc5c(c4)oc4ccccc45)nc(-c4ccc(-c5cccc6oc7ccccc7c56)c5c4oc4ccccc45)n3)cc2)c1 ; c1cncc(-c2cccc(-c3nc(-c4ccc5c(c4)oc4ccccc45)nc(-c4ccc(-c5cccc6oc7ccccc7c56)c5c4oc4ccccc45)n3)c2)c1 ; c1ccc(-c2cc(-c3ccccc3)cc(-c3cc(-c4ccccc4)nc(-c4ccc(-c5cccc6oc7ccccc7c56)c5c4oc4ccccc45)n3)c2)cc1]

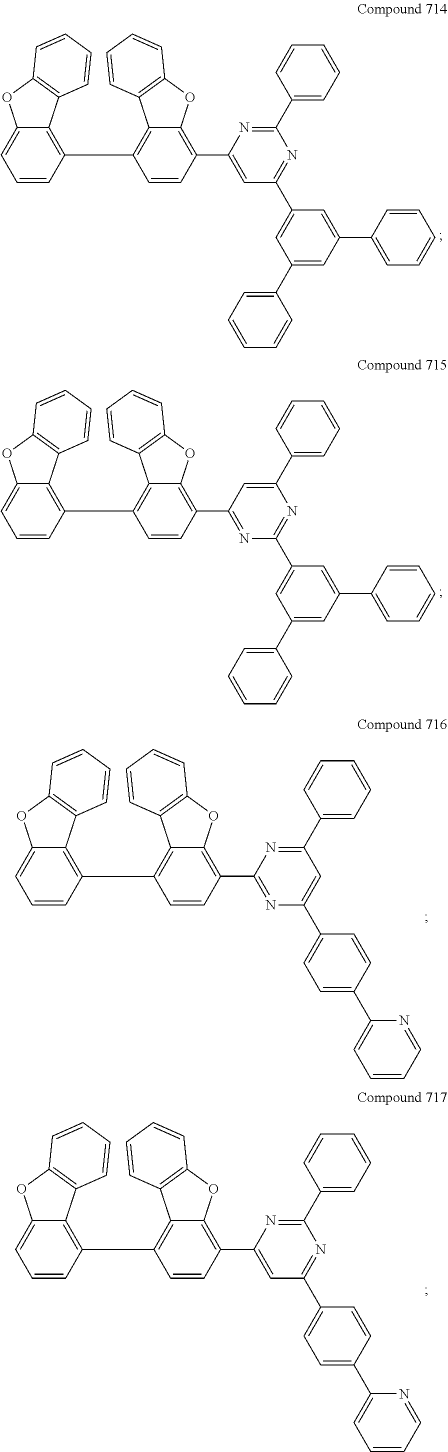 [SMILES: c1ccc(-c2cc(-c3ccccc3)cc(-c3cc(-c4ccc(-c5cccc6oc7ccccc7c56)c5c4oc4ccccc45)nc(-c4ccccc4)n3)c2)cc1 ; c1ccc(-c2cc(-c3ccccc3)cc(-c3nc(-c4ccccc4)cc(-c4ccc(-c5cccc6oc7ccccc7c56)c5c4oc4ccccc45)n3)c2)cc1 ; c1ccc(-c2cc(-c3ccc(-c4ccccn4)cc3)nc(-c3ccc(-c4cccc5oc6ccccc6c45)c4c3oc3ccccc34)n2)cc1 ; c1ccc(-c2nc(-c3ccc(-c4ccccn4)cc3)cc(-c3ccc(-c4cccc5oc6ccccc6c45)c4c3oc3ccccc34)n2)cc1]

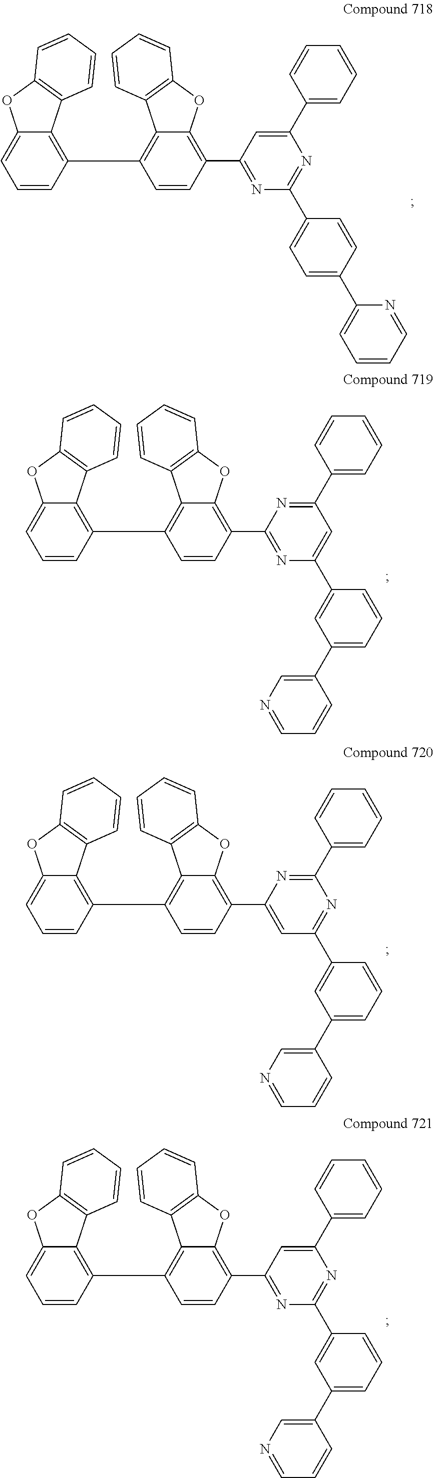 [SMILES: c1ccc(-c2cc(-c3ccc(-c4cccc5oc6ccccc6c45)c4c3oc3ccccc34)nc(-c3ccc(-c4ccccn4)cc3)n2)cc1 ; c1ccc(-c2cc(-c3cccc(-c4cccnc4)c3)nc(-c3ccc(-c4cccc5oc6ccccc6c45)c4c3oc3ccccc34)n2)cc1 ; c1ccc(-c2nc(-c3cccc(-c4cccnc4)c3)cc(-c3ccc(-c4cccc5oc6ccccc6c45)c4c3oc3ccccc34)n2)cc1 ; c1ccc(-c2cc(-c3ccc(-c4cccc5oc6ccccc6c45)c4c3oc3ccccc34)nc(-c3cccc(-c4cccnc4)c3)n2)cc1]

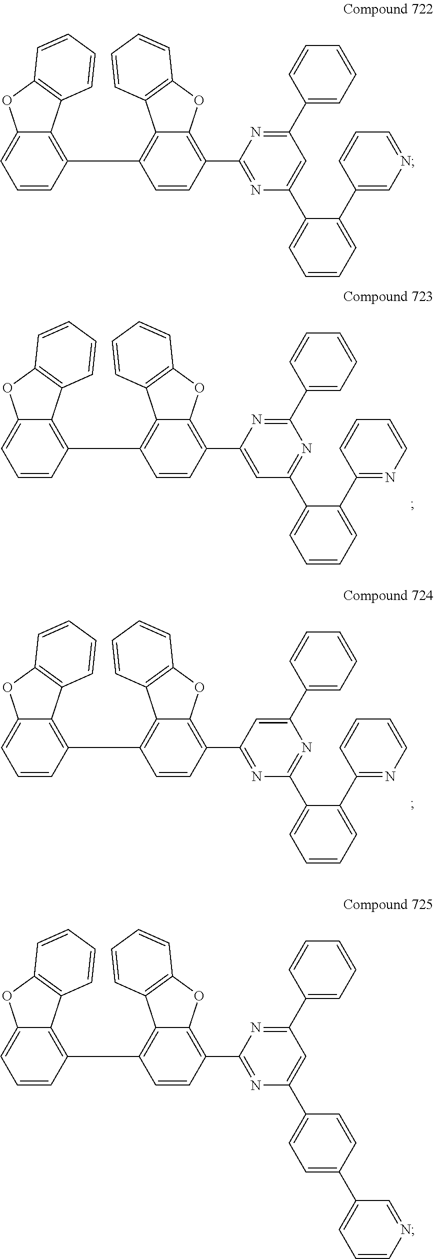 [SMILES: c1ccc(-c2cc(-c3ccccc3-c3cccnc3)nc(-c3ccc(-c4cccc5oc6ccccc6c45)c4c3oc3ccccc34)n2)cc1 ; c1ccc(-c2nc(-c3ccccc3-c3ccccn3)cc(-c3ccc(-c4cccc5oc6ccccc6c45)c4c3oc3ccccc34)n2)cc1 ; c1ccc(-c2cc(-c3ccc(-c4cccc5oc6ccccc6c45)c4c3oc3ccccc34)nc(-c3ccccc3-c3ccccn3)n2)cc1 ; c1ccc(-c2cc(-c3ccc(-c4cccnc4)cc3)nc(-c3ccc(-c4cccc5oc6ccccc6c45)c4c3oc3ccccc34)n2)cc1]

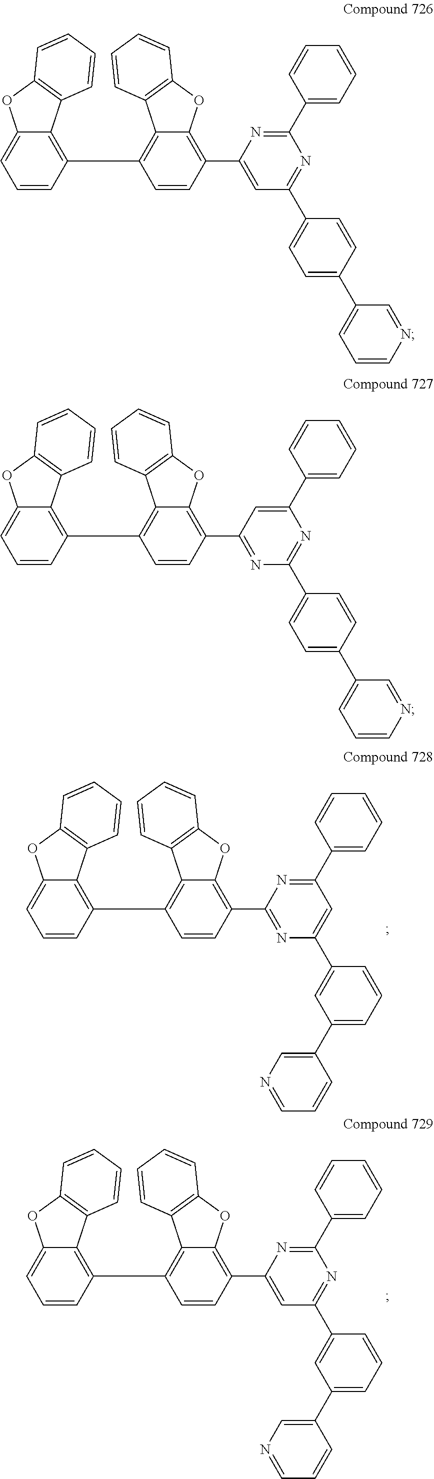 [SMILES: c1ccc(-c2nc(-c3ccc(-c4cccnc4)cc3)cc(-c3ccc(-c4cccc5oc6ccccc6c45)c4c3oc3ccccc34)n2)cc1 ; c1ccc(-c2cc(-c3ccc(-c4cccc5oc6ccccc6c45)c4c3oc3ccccc34)nc(-c3ccc(-c4cccnc4)cc3)n2)cc1 ; c1ccc(-c2cc(-c3cccc(-c4cccnc4)c3)nc(-c3ccc(-c4cccc5oc6ccccc6c45)c4c3oc3ccccc34)n2)cc1 ; c1ccc(-c2nc(-c3cccc(-c4cccnc4)c3)cc(-c3ccc(-c4cccc5oc6ccccc6c45)c4c3oc3ccccc34)n2)cc1]

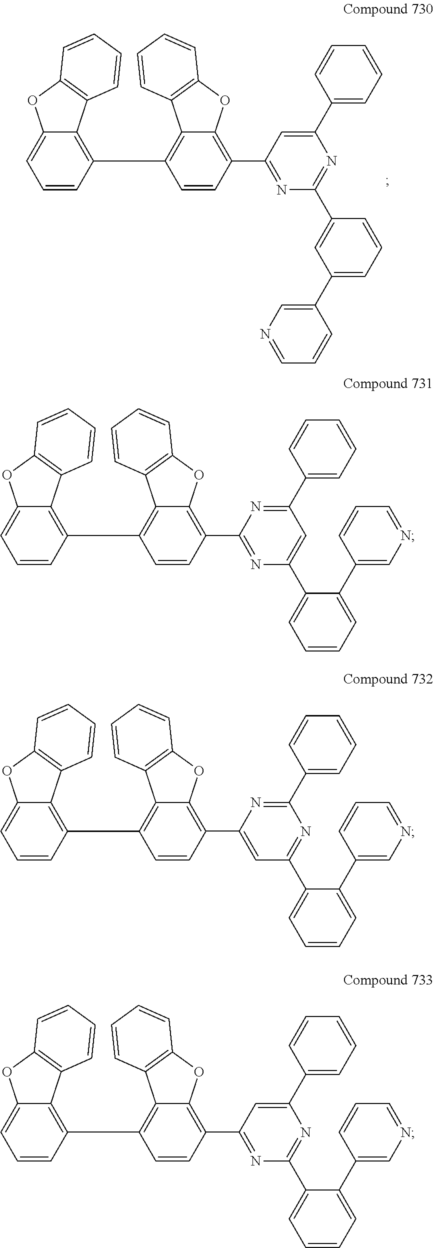 [SMILES: c1ccc(-c2cc(-c3ccc(-c4cccc5oc6ccccc6c45)c4c3oc3ccccc34)nc(-c3cccc(-c4cccnc4)c3)n2)cc1 ; c1ccc(-c2cc(-c3ccccc3-c3cccnc3)nc(-c3ccc(-c4cccc5oc6ccccc6c45)c4c3oc3ccccc34)n2)cc1 ; c1ccc(-c2nc(-c3ccccc3-c3cccnc3)cc(-c3ccc(-c4cccc5oc6ccccc6c45)c4c3oc3ccccc34)n2)cc1 ; c1ccc(-c2cc(-c3ccc(-c4cccc5oc6ccccc6c45)c4c3oc3ccccc34)nc(-c3ccccc3-c3cccnc3)n2)cc1]

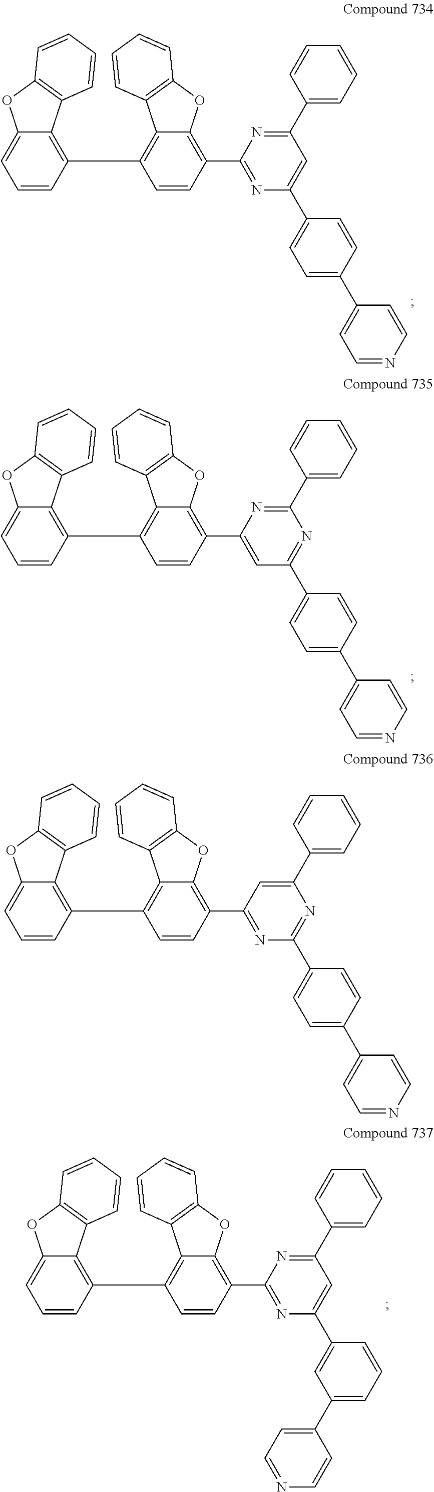 [SMILES: c1ccc(-c2cc(-c3ccc(-c4ccncc4)cc3)nc(-c3ccc(-c4cccc5oc6ccccc6c45)c4c3oc3ccccc34)n2)cc1 ; c1ccc(-c2nc(-c3ccc(-c4ccncc4)cc3)cc(-c3ccc(-c4cccc5oc6ccccc6c45)c4c3oc3ccccc34)n2)cc1 ; c1ccc(-c2cc(-c3ccc(-c4cccc5oc6ccccc6c45)c4c3oc3ccccc34)nc(-c3ccc(-c4ccncc4)cc3)n2)cc1 ; c1ccc(-c2cc(-c3cccc(-c4ccncc4)c3)nc(-c3ccc(-c4cccc5oc6ccccc6c45)c4c3oc3ccccc34)n2)cc1]

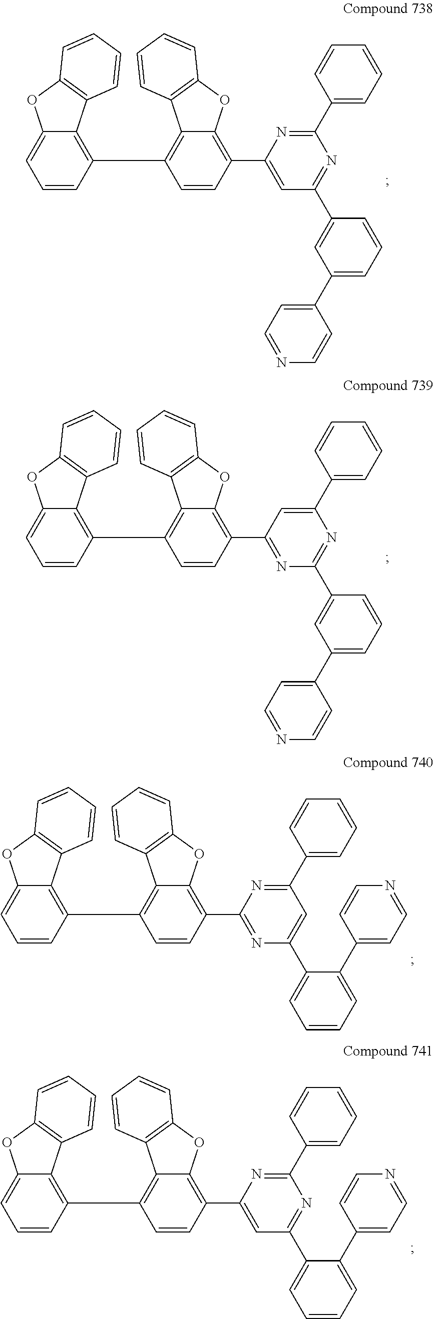 [SMILES: c1ccc(-c2nc(-c3cccc(-c4ccncc4)c3)cc(-c3ccc(-c4cccc5oc6ccccc6c45)c4c3oc3ccccc34)n2)cc1 ; c1ccc(-c2cc(-c3ccc(-c4cccc5oc6ccccc6c45)c4c3oc3ccccc34)nc(-c3cccc(-c4ccncc4)c3)n2)cc1 ; c1ccc(-c2cc(-c3ccccc3-c3ccncc3)nc(-c3ccc(-c4cccc5oc6ccccc6c45)c4c3oc3ccccc34)n2)cc1 ; c1ccc(-c2nc(-c3ccccc3-c3ccncc3)cc(-c3ccc(-c4cccc5oc6ccccc6c45)c4c3oc3ccccc34)n2)cc1]

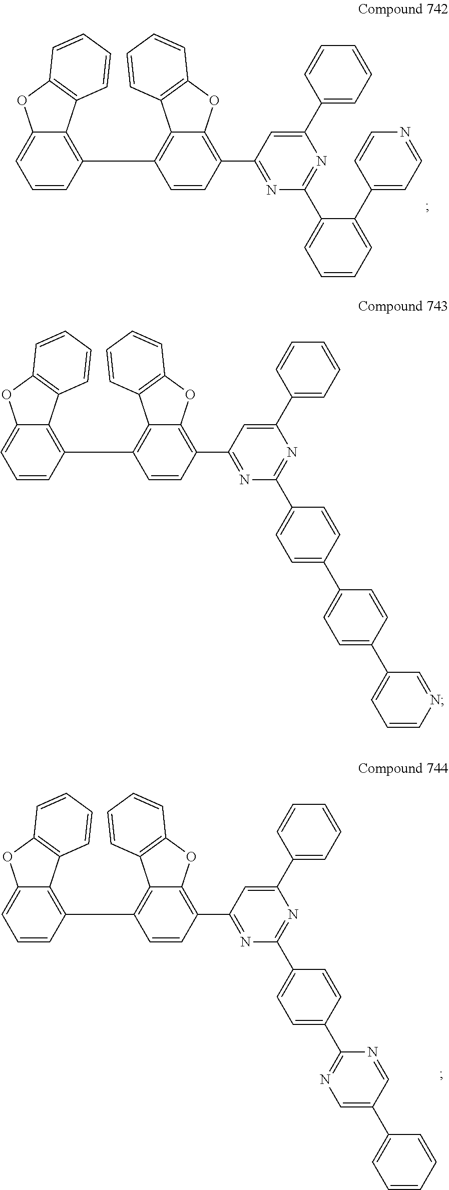 [SMILES: c1ccc(-c2cc(-c3ccc(-c4cccc5oc6ccccc6c45)c4c3oc3ccccc34)nc(-c3ccccc3-c3ccncc3)n2)cc1 ; c1ccc(-c2cc(-c3ccc(-c4cccc5oc6ccccc6c45)c4c3oc3ccccc34)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6cccc7oc8ccccc8c67)c6c5oc5ccccc56)n4)cc3)nc2)cc1]

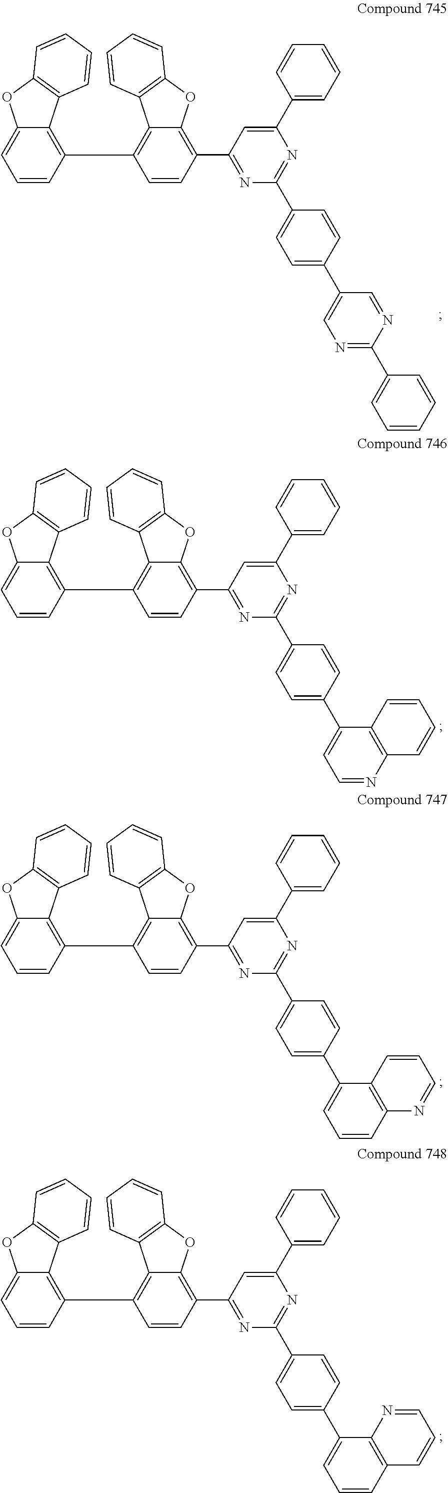 [SMILES: c1ccc(-c2cc(-c3ccc(-c4cccc5oc6ccccc6c45)c4c3oc3ccccc34)nc(-c3ccc(-c4cnc(-c5ccccc5)nc4)cc3)n2)cc1 ; c1ccc(-c2cc(-c3ccc(-c4cccc5oc6ccccc6c45)c4c3oc3ccccc34)nc(-c3ccc(-c4ccnc5ccccc45)cc3)n2)cc1 ; c1ccc(-c2cc(-c3ccc(-c4cccc5oc6ccccc6c45)c4c3oc3ccccc34)nc(-c3ccc(-c4cccc5ncccc45)cc3)n2)cc1 ; c1ccc(-c2cc(-c3ccc(-c4cccc5oc6ccccc6c45)c4c3oc3ccccc34)nc(-c3ccc(-c4cccc5cccnc45)cc3)n2)cc1]

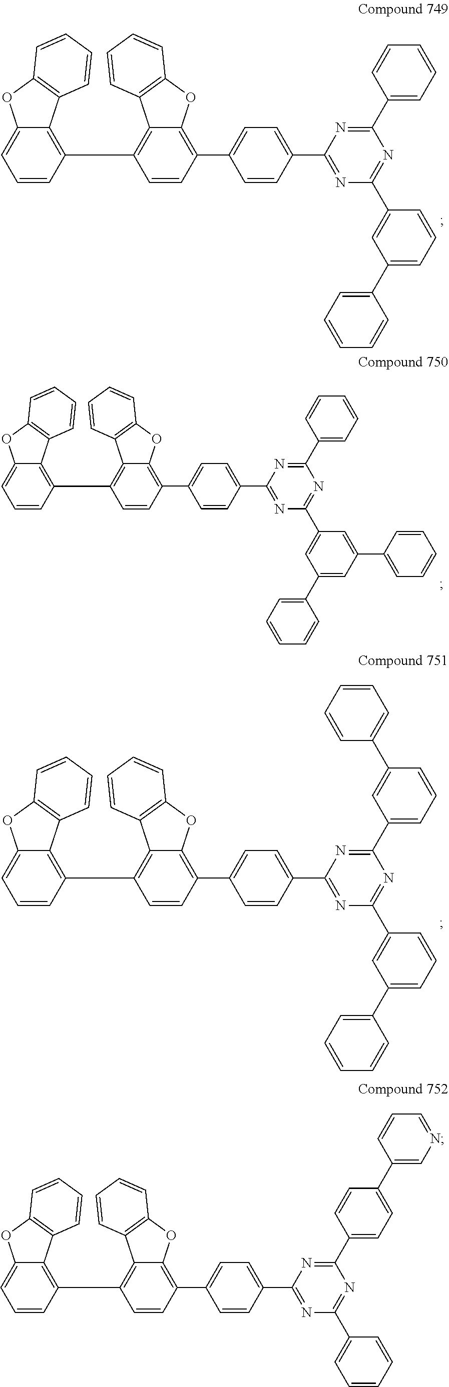 [SMILES: c1ccc(-c2cccc(-c3nc(-c4ccccc4)nc(-c4ccc(-c5ccc(-c6cccc7oc8ccccc8c67)c6c5oc5ccccc56)cc4)n3)c2)cc1 ; c1ccc(-c2cc(-c3ccccc3)cc(-c3nc(-c4ccccc4)nc(-c4ccc(-c5ccc(-c6cccc7oc8ccccc8c67)c6c5oc5ccccc56)cc4)n3)c2)cc1 ; c1ccc(-c2cccc(-c3nc(-c4ccc(-c5ccc(-c6cccc7oc8ccccc8c67)c6c5oc5ccccc56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5cccc6oc7ccccc7c56)c5c4oc4ccccc45)cc3)n2)cc1]

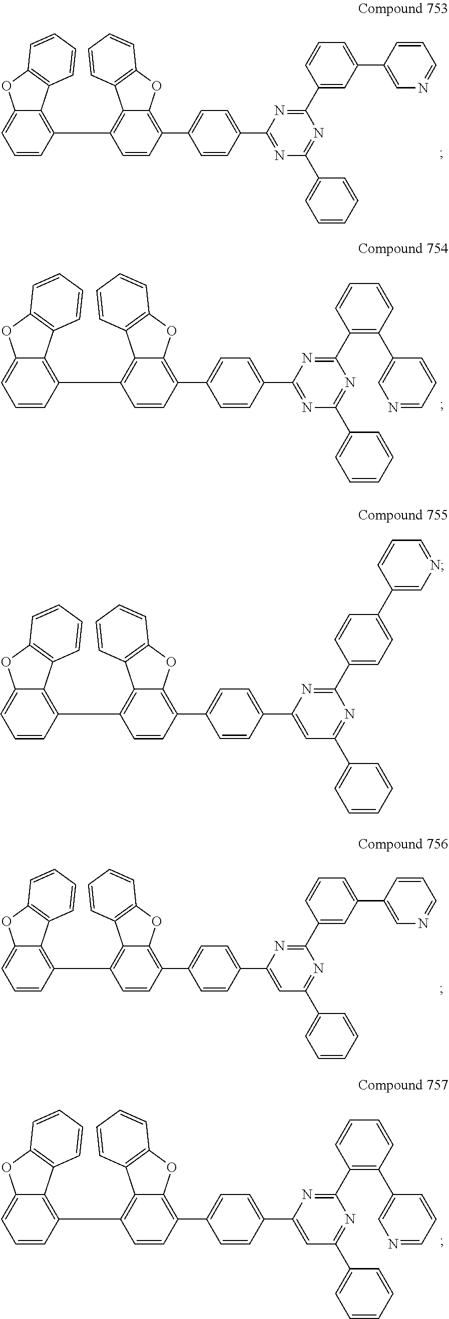 [SMILES: c1ccc(-c2nc(-c3ccc(-c4ccc(-c5cccc6oc7ccccc7c56)c5c4oc4ccccc45)cc3)nc(-c3cccc(-c4cccnc4)c3)n2)cc1 ; c1ccc(-c2nc(-c3ccc(-c4ccc(-c5cccc6oc7ccccc7c56)c5c4oc4ccccc45)cc3)nc(-c3ccccc3-c3cccnc3)n2)cc1 ; c1ccc(-c2cc(-c3ccc(-c4ccc(-c5cccc6oc7ccccc7c56)c5c4oc4ccccc45)cc3)nc(-c3ccc(-c4cccnc4)cc3)n2)cc1 ; c1ccc(-c2cc(-c3ccc(-c4ccc(-c5cccc6oc7ccccc7c56)c5c4oc4ccccc45)cc3)nc(-c3cccc(-c4cccnc4)c3)n2)cc1 ; c1ccc(-c2cc(-c3ccc(-c4ccc(-c5cccc6oc7ccccc7c56)c5c4oc4ccccc45)cc3)nc(-c3ccccc3-c3cccnc3)n2)cc1]

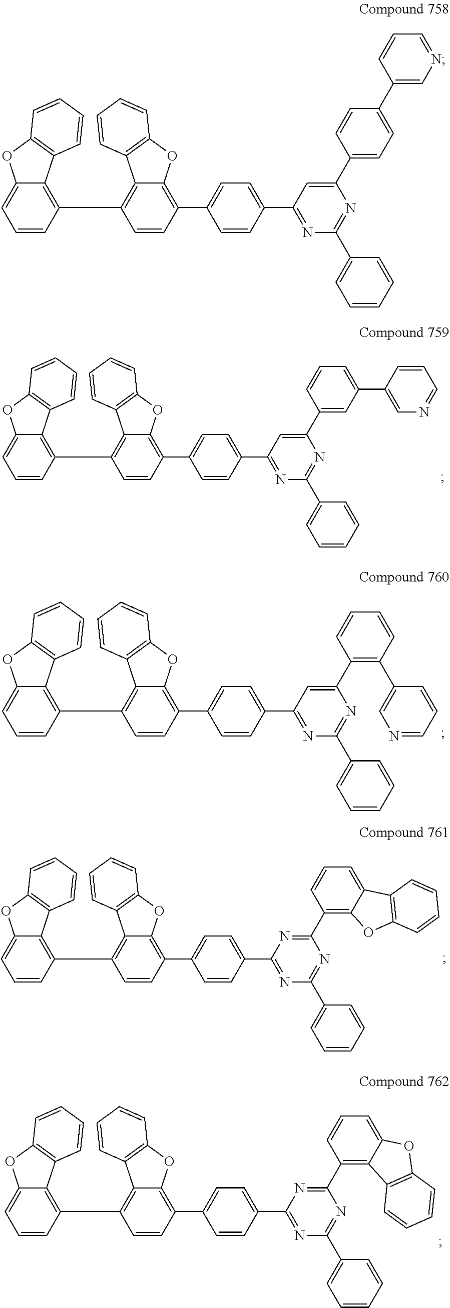 [SMILES: c1ccc(-c2nc(-c3ccc(-c4cccnc4)cc3)cc(-c3ccc(-c4ccc(-c5cccc6oc7ccccc7c56)c5c4oc4ccccc45)cc3)n2)cc1 ; c1ccc(-c2nc(-c3ccc(-c4ccc(-c5cccc6oc7ccccc7c56)c5c4oc4ccccc45)cc3)cc(-c3cccc(-c4cccnc4)c3)n2)cc1 ; c1ccc(-c2nc(-c3ccc(-c4ccc(-c5cccc6oc7ccccc7c56)c5c4oc4ccccc45)cc3)cc(-c3ccccc3-c3cccnc3)n2)cc1 ; c1ccc(-c2nc(-c3ccc(-c4ccc(-c5cccc6oc7ccccc7c56)c5c4oc4ccccc45)cc3)nc(-c3cccc4c3oc3ccccc34)n2)cc1 ; c1ccc(-c2nc(-c3ccc(-c4ccc(-c5cccc6oc7ccccc7c56)c5c4oc4ccccc45)cc3)nc(-c3cccc4oc5ccccc5c34)n2)cc1]

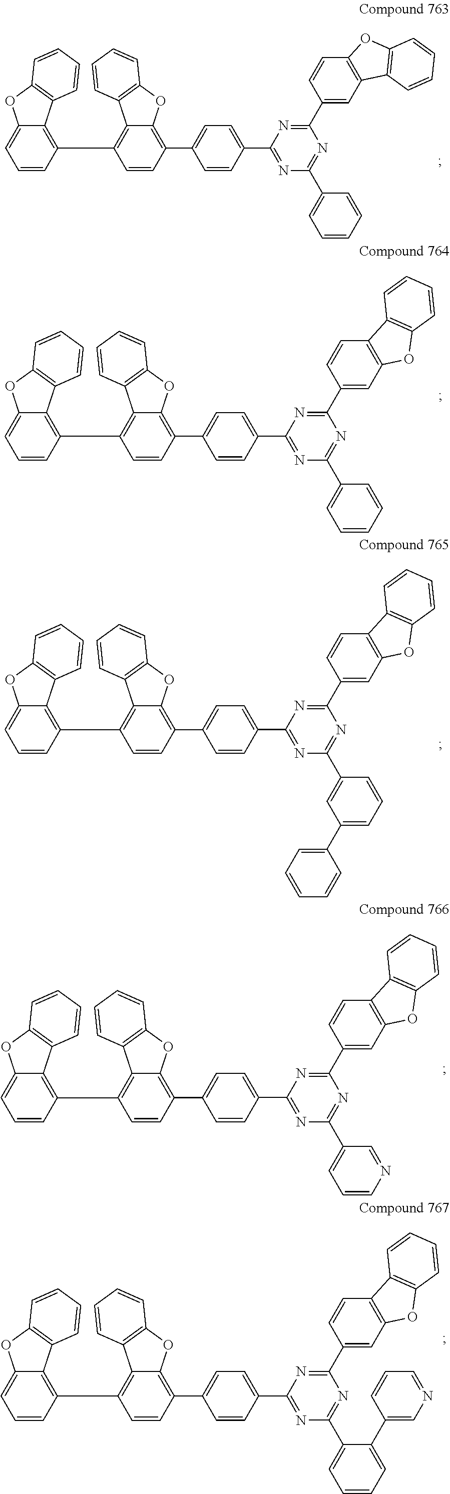

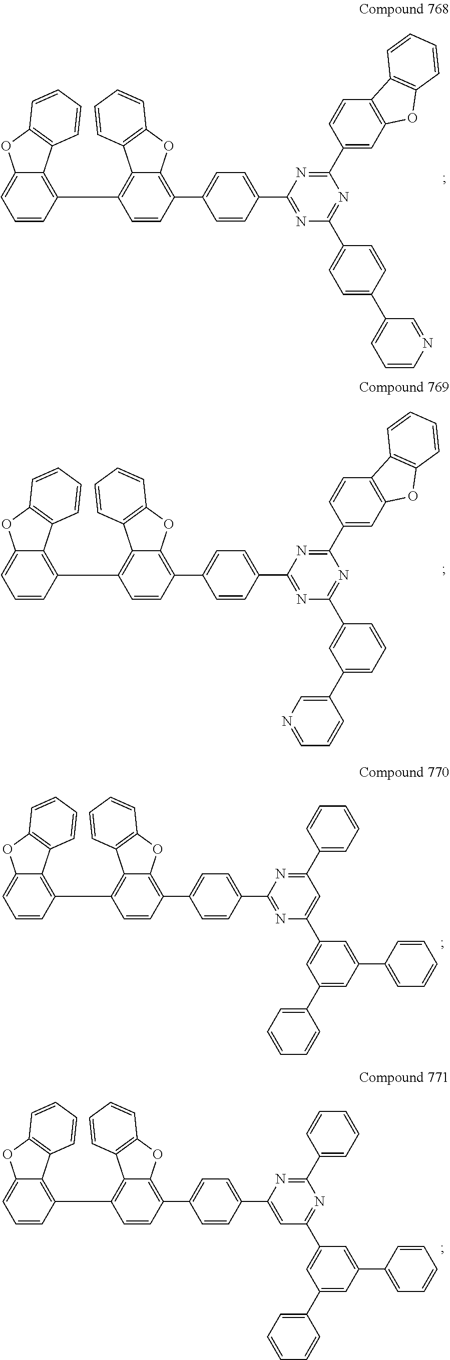 [SMILES: c1cncc(-c2ccc(-c3nc(-c4ccc(-c5ccc(-c6cccc7oc8ccccc8c67)c6c5oc5ccccc56)cc4)nc(-c4ccc5c(c4)oc4ccccc45)n3)cc2)c1 ; c1cncc(-c2cccc(-c3nc(-c4ccc(-c5ccc(-c6cccc7oc8ccccc8c67)c6c5oc5ccccc56)cc4)nc(-c4ccc5c(c4)oc4ccccc45)n3)c2)c1 ; c1ccc(-c2cc(-c3ccccc3)cc(-c3cc(-c4ccccc4)nc(-c4ccc(-c5ccc(-c6cccc7oc8ccccc8c67)c6c5oc5ccccc56)cc4)n3)c2)cc1 ; c1ccc(-c2cc(-c3ccccc3)cc(-c3cc(-c4ccc(-c5ccc(-c6cccc7oc8ccccc8c67)c6c5oc5ccccc56)cc4)nc(-c4ccccc4)n3)c2)cc1]

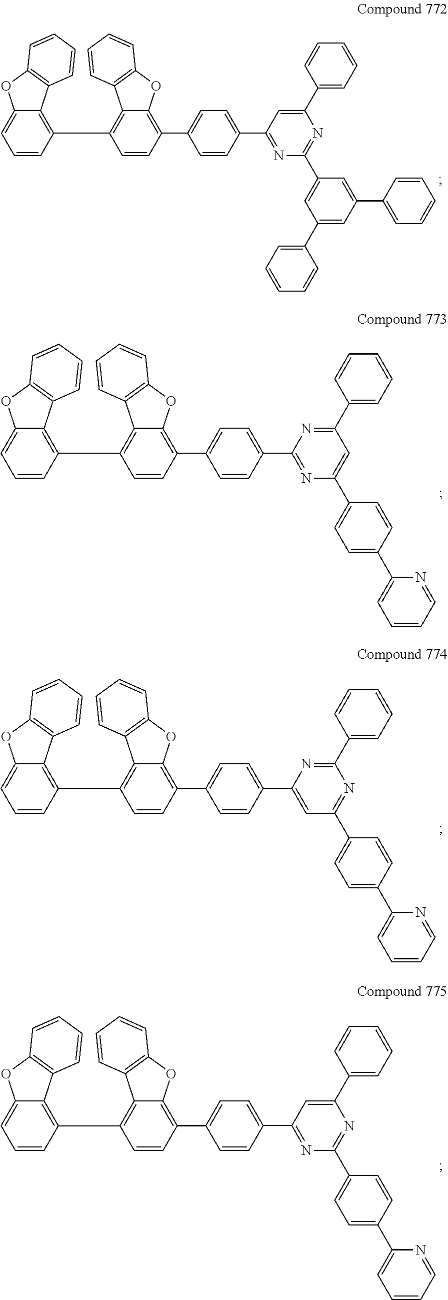 [SMILES: c1ccc(-c2cc(-c3ccccc3)cc(-c3nc(-c4ccccc4)cc(-c4ccc(-c5ccc(-c6cccc7oc8ccccc8c67)c6c5oc5ccccc56)cc4)n3)c2)cc1 ; c1ccc(-c2cc(-c3ccc(-c4ccccn4)cc3)nc(-c3ccc(-c4ccc(-c5cccc6oc7ccccc7c56)c5c4oc4ccccc45)cc3)n2)cc1 ; c1ccc(-c2nc(-c3ccc(-c4ccccn4)cc3)cc(-c3ccc(-c4ccc(-c5cccc6oc7ccccc7c56)c5c4oc4ccccc45)cc3)n2)cc1 ; c1ccc(-c2cc(-c3ccc(-c4ccc(-c5cccc6oc7ccccc7c56)c5c4oc4ccccc45)cc3)nc(-c3ccc(-c4ccccn4)cc3)n2)cc1]

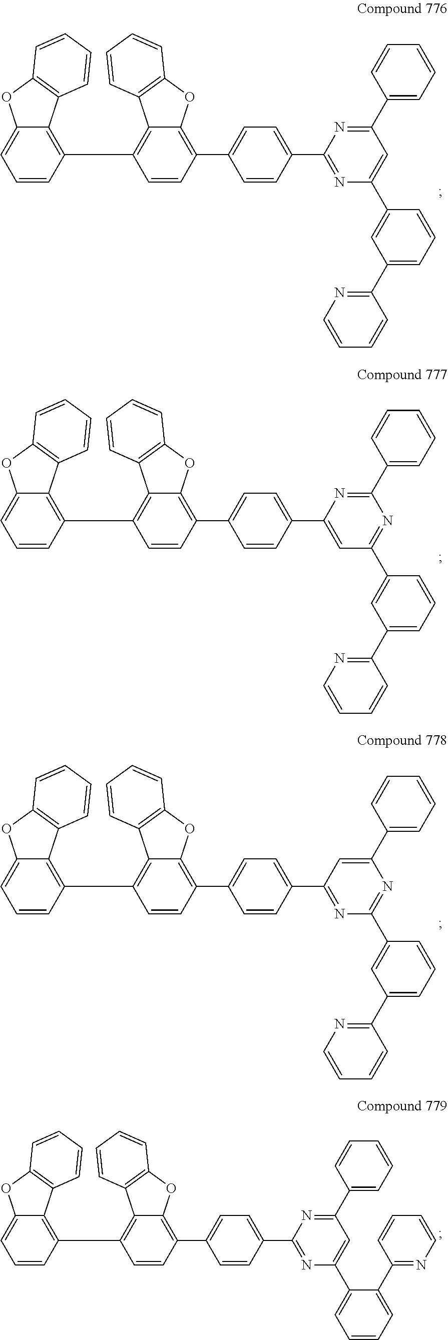 [SMILES: c1ccc(-c2cc(-c3cccc(-c4ccccn4)c3)nc(-c3ccc(-c4ccc(-c5cccc6oc7ccccc7c56)c5c4oc4ccccc45)cc3)n2)cc1 ; c1ccc(-c2nc(-c3ccc(-c4ccc(-c5cccc6oc7ccccc7c56)c5c4oc4ccccc45)cc3)cc(-c3cccc(-c4ccccn4)c3)n2)cc1 ; c1ccc(-c2cc(-c3ccc(-c4ccc(-c5cccc6oc7ccccc7c56)c5c4oc4ccccc45)cc3)nc(-c3cccc(-c4ccccn4)c3)n2)cc1 ; c1ccc(-c2cc(-c3ccccc3-c3ccccn3)nc(-c3ccc(-c4ccc(-c5cccc6oc7ccccc7c56)c5c4oc4ccccc45)cc3)n2)cc1]

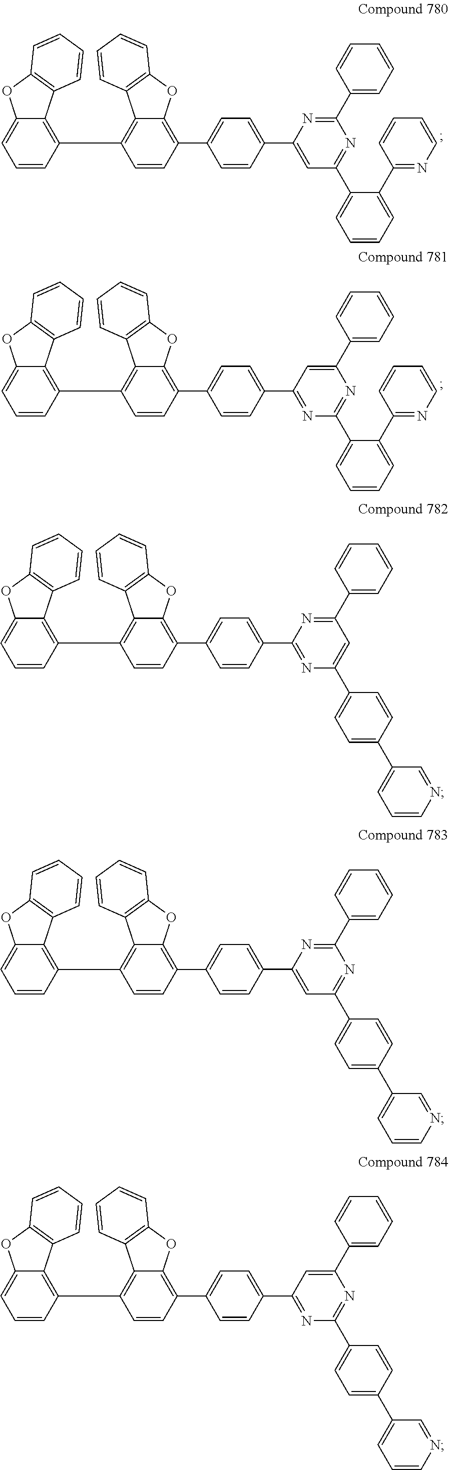 [SMILES: c1ccc(-c2nc(-c3ccc(-c4ccc(-c5cccc6oc7ccccc7c56)c5c4oc4ccccc45)cc3)cc(-c3ccccc3-c3ccccn3)n2)cc1 ; c1ccc(-c2cc(-c3ccc(-c4ccc(-c5cccc6oc7ccccc7c56)c5c4oc4ccccc45)cc3)nc(-c3ccccc3-c3ccccn3)n2)cc1 ; c1ccc(-c2cc(-c3ccc(-c4cccnc4)cc3)nc(-c3ccc(-c4ccc(-c5cccc6oc7ccccc7c56)c5c4oc4ccccc45)cc3)n2)cc1 ; c1ccc(-c2nc(-c3ccc(-c4cccnc4)cc3)cc(-c3ccc(-c4ccc(-c5cccc6oc7ccccc7c56)c5c4oc4ccccc45)cc3)n2)cc1 ; c1ccc(-c2cc(-c3ccc(-c4ccc(-c5cccc6oc7ccccc7c56)c5c4oc4ccccc45)cc3)nc(-c3ccc(-c4cccnc4)cc3)n2)cc1]

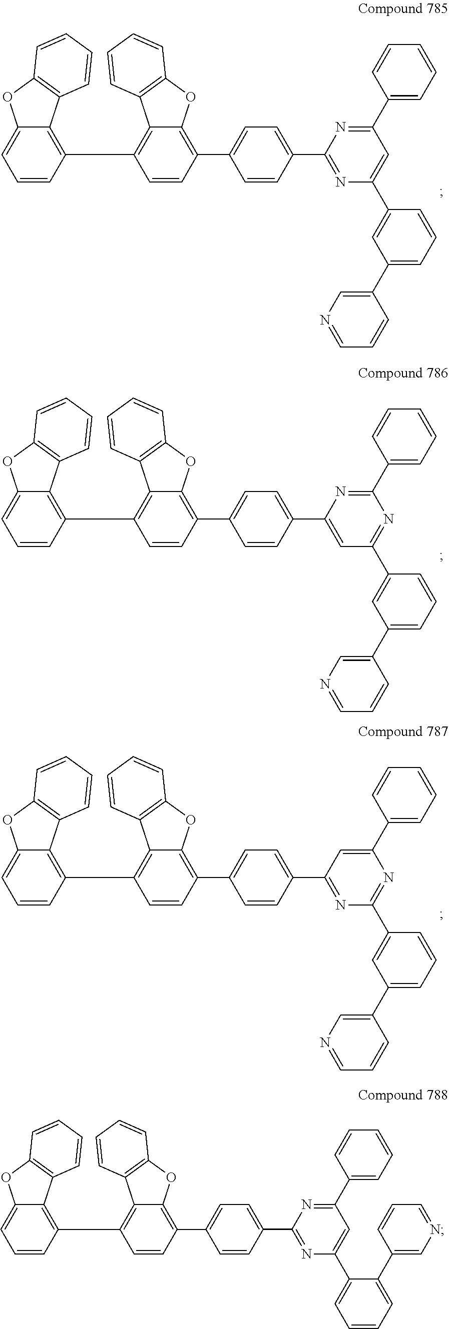 [SMILES: c1ccc(-c2cc(-c3cccc(-c4cccnc4)c3)nc(-c3ccc(-c4ccc(-c5cccc6oc7ccccc7c56)c5c4oc4ccccc45)cc3)n2)cc1 ; c1ccc(-c2nc(-c3ccc(-c4ccc(-c5cccc6oc7ccccc7c56)c5c4oc4ccccc45)cc3)cc(-c3cccc(-c4cccnc4)c3)n2)cc1 ; c1ccc(-c2cc(-c3ccc(-c4ccc(-c5cccc6oc7ccccc7c56)c5c4oc4ccccc45)cc3)nc(-c3cccc(-c4cccnc4)c3)n2)cc1 ; c1ccc(-c2cc(-c3ccccc3-c3cccnc3)nc(-c3ccc(-c4ccc(-c5cccc6oc7ccccc7c56)c5c4oc4ccccc45)cc3)n2)cc1]

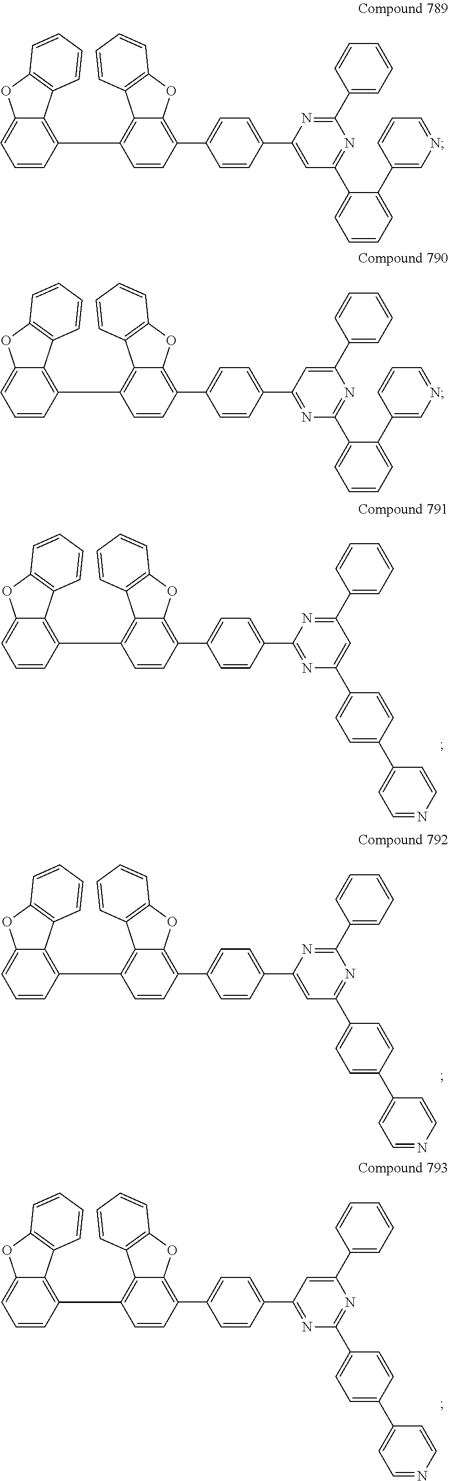 [SMILES: c1ccc(-c2nc(-c3ccc(-c4ccc(-c5cccc6oc7ccccc7c56)c5c4oc4ccccc45)cc3)cc(-c3ccccc3-c3cccnc3)n2)cc1 ; c1ccc(-c2cc(-c3ccc(-c4ccc(-c5cccc6oc7ccccc7c56)c5c4oc4ccccc45)cc3)nc(-c3ccccc3-c3cccnc3)n2)cc1 ; c1ccc(-c2cc(-c3ccc(-c4ccncc4)cc3)nc(-c3ccc(-c4ccc(-c5cccc6oc7ccccc7c56)c5c4oc4ccccc45)cc3)n2)cc1 ; c1ccc(-c2nc(-c3ccc(-c4ccncc4)cc3)cc(-c3ccc(-c4ccc(-c5cccc6oc7ccccc7c56)c5c4oc4ccccc45)cc3)n2)cc1 ; c1ccc(-c2cc(-c3ccc(-c4ccc(-c5cccc6oc7ccccc7c56)c5c4oc4ccccc45)cc3)nc(-c3ccc(-c4ccncc4)cc3)n2)cc1]

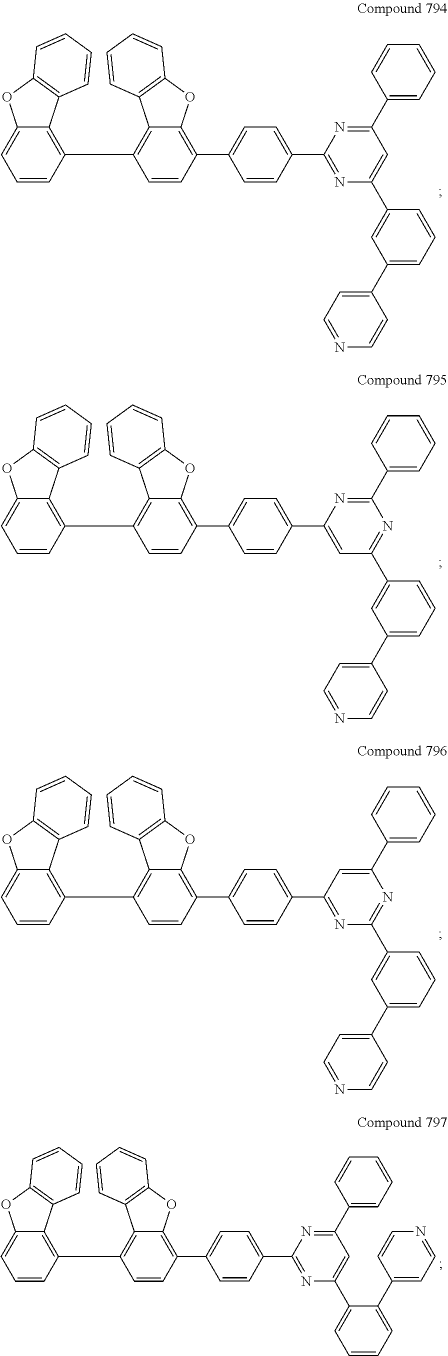 [SMILES: c1ccc(-c2cc(-c3cccc(-c4ccncc4)c3)nc(-c3ccc(-c4ccc(-c5cccc6oc7ccccc7c56)c5c4oc4ccccc45)cc3)n2)cc1 ; c1ccc(-c2nc(-c3ccc(-c4ccc(-c5cccc6oc7ccccc7c56)c5c4oc4ccccc45)cc3)cc(-c3cccc(-c4ccncc4)c3)n2)cc1 ; c1ccc(-c2cc(-c3ccc(-c4ccc(-c5cccc6oc7ccccc7c56)c5c4oc4ccccc45)cc3)nc(-c3cccc(-c4ccncc4)c3)n2)cc1 ; c1ccc(-c2cc(-c3ccccc3-c3ccncc3)nc(-c3ccc(-c4ccc(-c5cccc6oc7ccccc7c56)c5c4oc4ccccc45)cc3)n2)cc1]

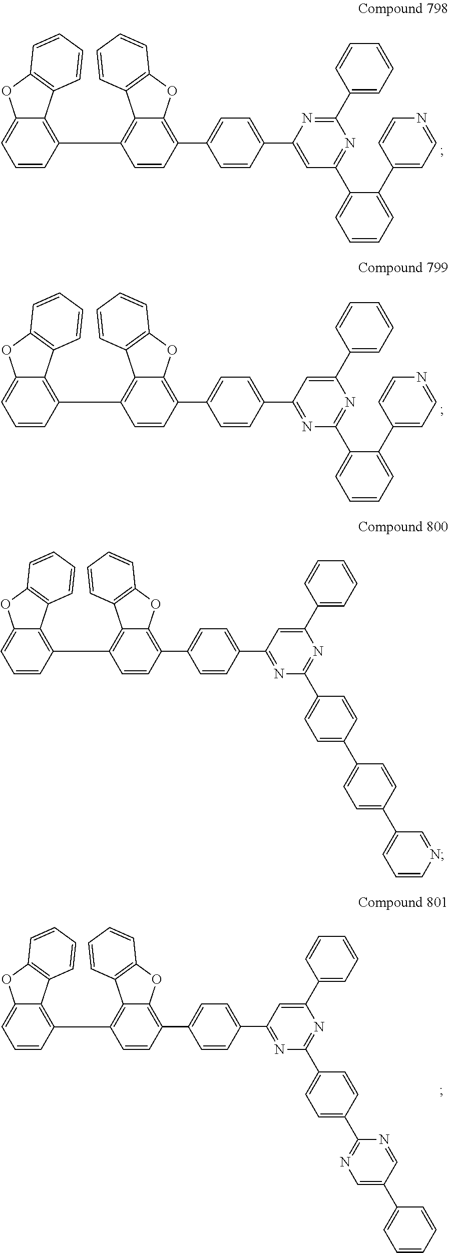 [SMILES: c1ccc(-c2nc(-c3ccc(-c4ccc(-c5cccc6oc7ccccc7c56)c5c4oc4ccccc45)cc3)cc(-c3ccccc3-c3ccncc3)n2)cc1 ; c1ccc(-c2cc(-c3ccc(-c4ccc(-c5cccc6oc7ccccc7c56)c5c4oc4ccccc45)cc3)nc(-c3ccccc3-c3ccncc3)n2)cc1 ; c1ccc(-c2cc(-c3ccc(-c4ccc(-c5cccc6oc7ccccc7c56)c5c4oc4ccccc45)cc3)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc(-c7cccc8oc9ccccc9c78)c7c6oc6ccccc67)cc5)n4)cc3)nc2)cc1]

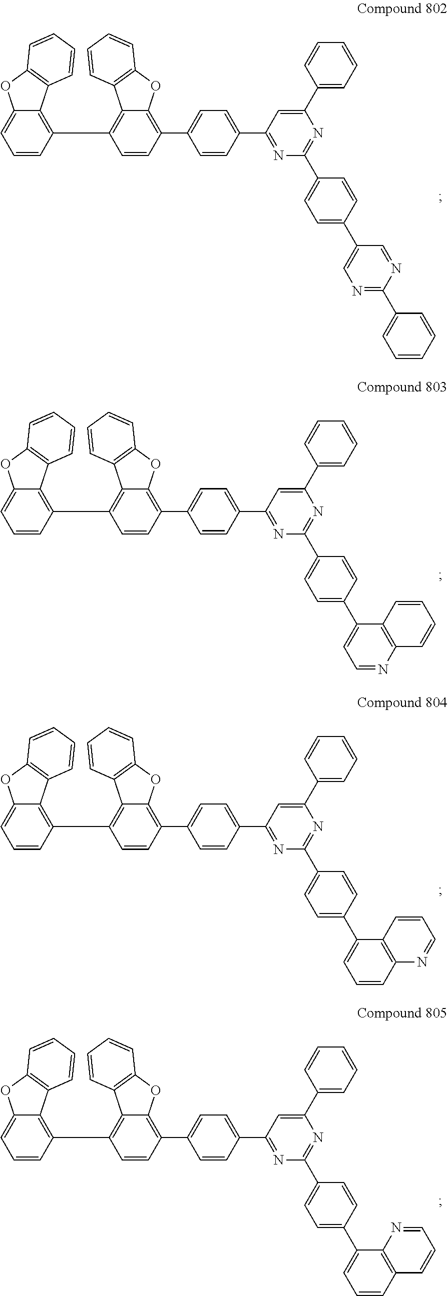 [SMILES: c1ccc(-c2cc(-c3ccc(-c4ccc(-c5cccc6oc7ccccc7c56)c5c4oc4ccccc45)cc3)nc(-c3ccc(-c4cnc(-c5ccccc5)nc4)cc3)n2)cc1 ; c1ccc(-c2cc(-c3ccc(-c4ccc(-c5cccc6oc7ccccc7c56)c5c4oc4ccccc45)cc3)nc(-c3ccc(-c4ccnc5ccccc45)cc3)n2)cc1 ; c1ccc(-c2cc(-c3ccc(-c4ccc(-c5cccc6oc7ccccc7c56)c5c4oc4ccccc45)cc3)nc(-c3ccc(-c4cccc5ncccc45)cc3)n2)cc1 ; c1ccc(-c2cc(-c3ccc(-c4ccc(-c5cccc6oc7ccccc7c56)c5c4oc4ccccc45)cc3)nc(-c3ccc(-c4cccc5cccnc45)cc3)n2)cc1]

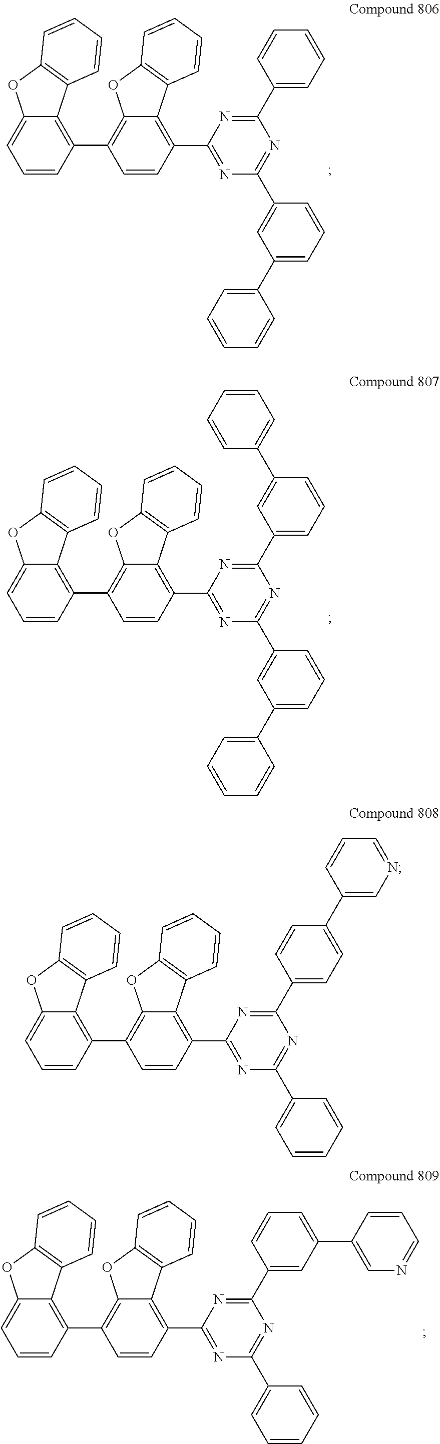

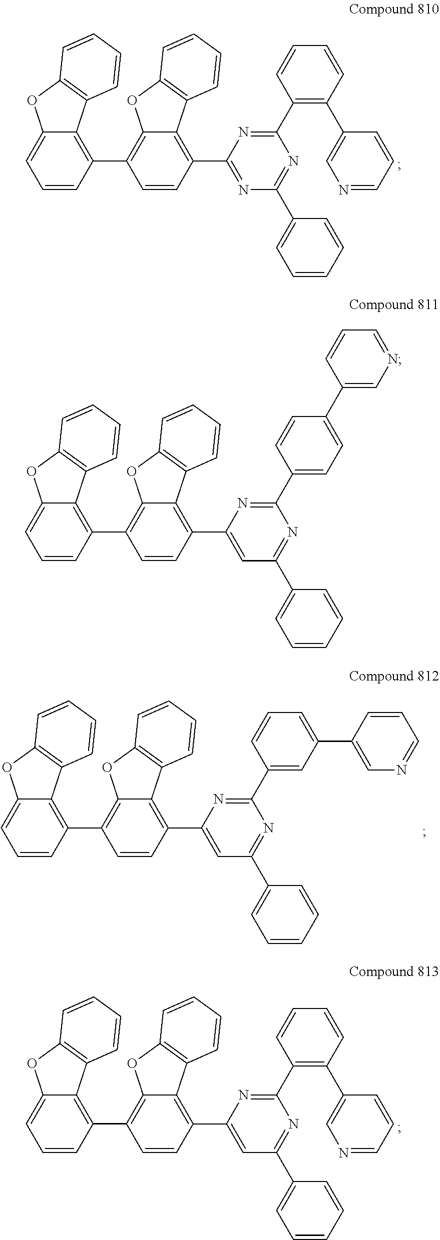 [SMILES: c1ccc(-c2nc(-c3ccccc3-c3cccnc3)nc(-c3ccc(-c4cccc5oc6ccccc6c45)c4oc5ccccc5c34)n2)cc1 ; c1ccc(-c2cc(-c3ccc(-c4cccc5oc6ccccc6c45)c4oc5ccccc5c34)nc(-c3ccc(-c4cccnc4)cc3)n2)cc1 ; c1ccc(-c2cc(-c3ccc(-c4cccc5oc6ccccc6c45)c4oc5ccccc5c34)nc(-c3cccc(-c4cccnc4)c3)n2)cc1 ; c1ccc(-c2cc(-c3ccc(-c4cccc5oc6ccccc6c45)c4oc5ccccc5c34)nc(-c3ccccc3-c3cccnc3)n2)cc1]

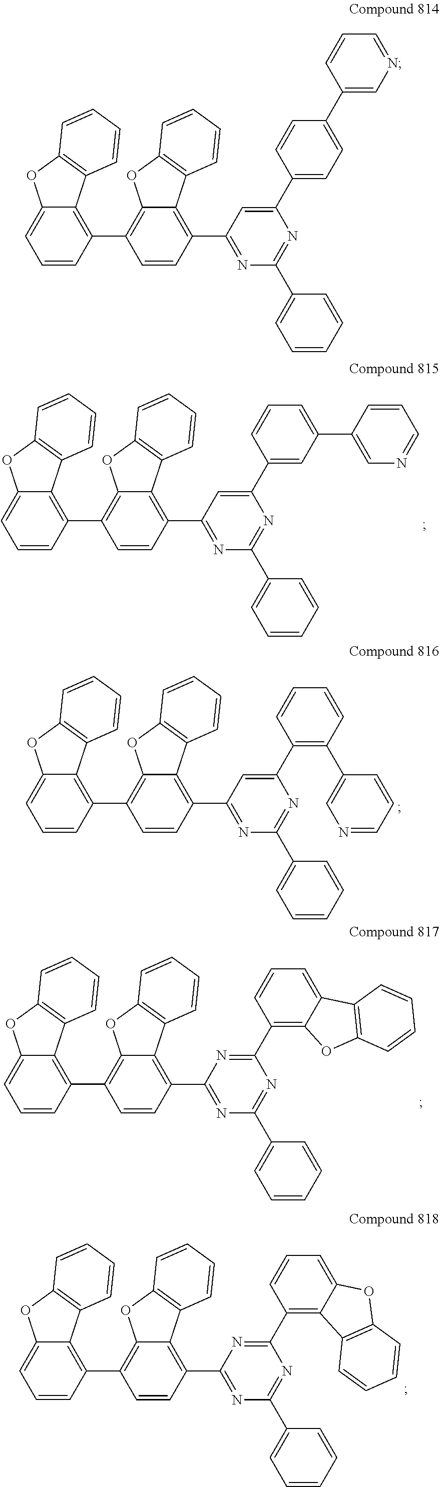 [SMILES: c1ccc(-c2nc(-c3ccc(-c4cccnc4)cc3)cc(-c3ccc(-c4cccc5oc6ccccc6c45)c4oc5ccccc5c34)n2)cc1 ; c1ccc(-c2nc(-c3cccc(-c4cccnc4)c3)cc(-c3ccc(-c4cccc5oc6ccccc6c45)c4oc5ccccc5c34)n2)cc1 ; c1ccc(-c2nc(-c3ccccc3-c3cccnc3)cc(-c3ccc(-c4cccc5oc6ccccc6c45)c4oc5ccccc5c34)n2)cc1 ; c1ccc(-c2nc(-c3cccc4c3oc3ccccc34)nc(-c3ccc(-c4cccc5oc6ccccc6c45)c4oc5ccccc5c34)n2)cc1 ; c1ccc(-c2nc(-c3cccc4oc5ccccc5c34)nc(-c3ccc(-c4cccc5oc6ccccc6c45)c4oc5ccccc5c34)n2)cc1]

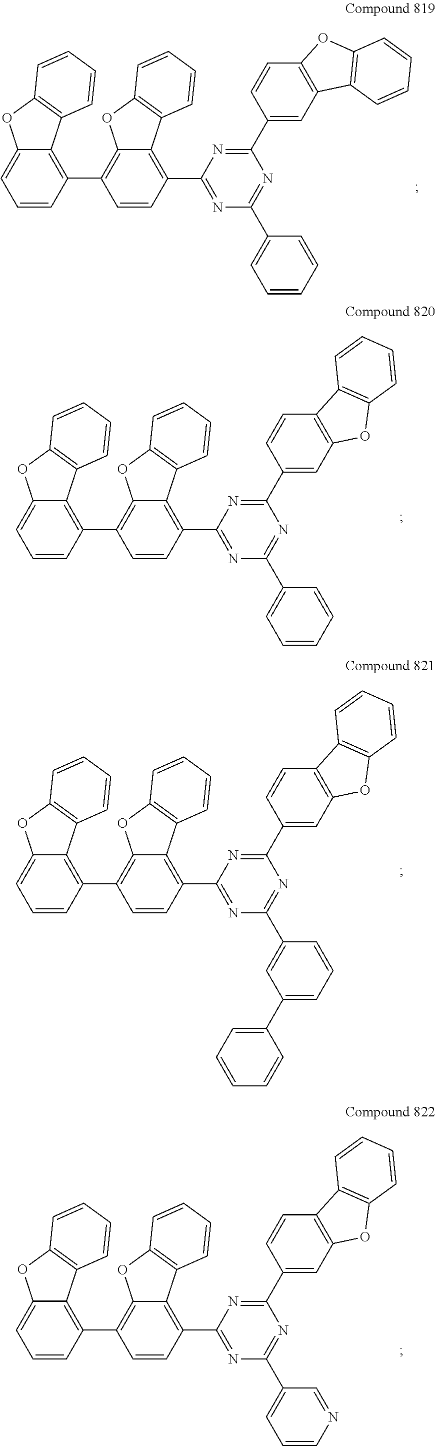 [SMILES: c1ccc(-c2nc(-c3ccc4oc5ccccc5c4c3)nc(-c3ccc(-c4cccc5oc6ccccc6c45)c4oc5ccccc5c34)n2)cc1 ; c1ccc(-c2nc(-c3ccc4c(c3)oc3ccccc34)nc(-c3ccc(-c4cccc5oc6ccccc6c45)c4oc5ccccc5c34)n2)cc1 ; c1ccc(-c2cccc(-c3nc(-c4ccc5c(c4)oc4ccccc45)nc(-c4ccc(-c5cccc6oc7ccccc7c56)c5oc6ccccc6c45)n3)c2)cc1 ; c1cncc(-c2nc(-c3ccc4c(c3)oc3ccccc34)nc(-c3ccc(-c4cccc5oc6ccccc6c45)c4oc5ccccc5c34)n2)c1]

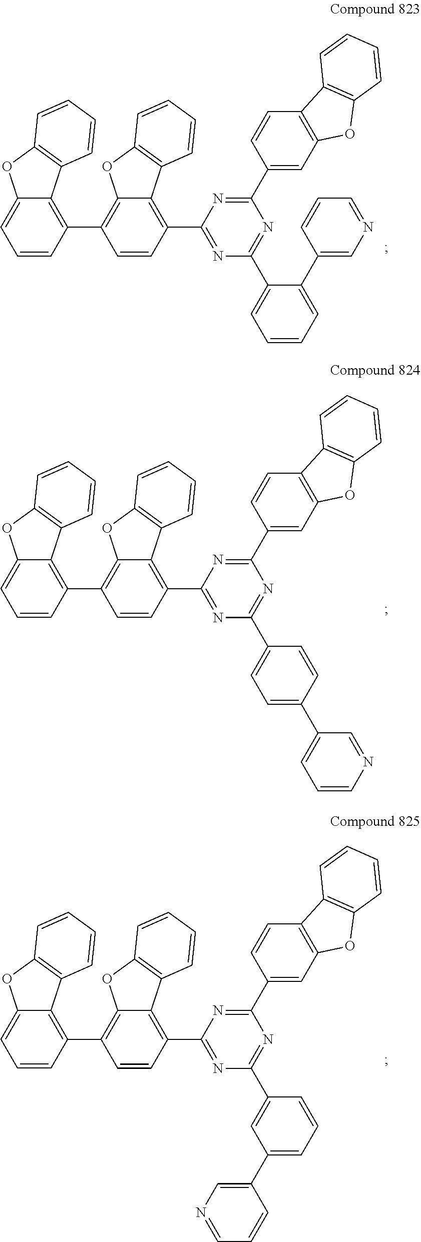 [SMILES: c1cncc(-c2ccccc2-c2nc(-c3ccc4c(c3)oc3ccccc34)nc(-c3ccc(-c4cccc5oc6ccccc6c45)c4oc5ccccc5c34)n2)c1 ; c1cncc(-c2ccc(-c3nc(-c4ccc5c(c4)oc4ccccc45)nc(-c4ccc(-c5cccc6oc7ccccc7c56)c5oc6ccccc6c45)n3)cc2)c1 ; c1cncc(-c2cccc(-c3nc(-c4ccc5c(c4)oc4ccccc45)nc(-c4ccc(-c5cccc6oc7ccccc7c56)c5oc6ccccc6c45)n3)c2)c1]

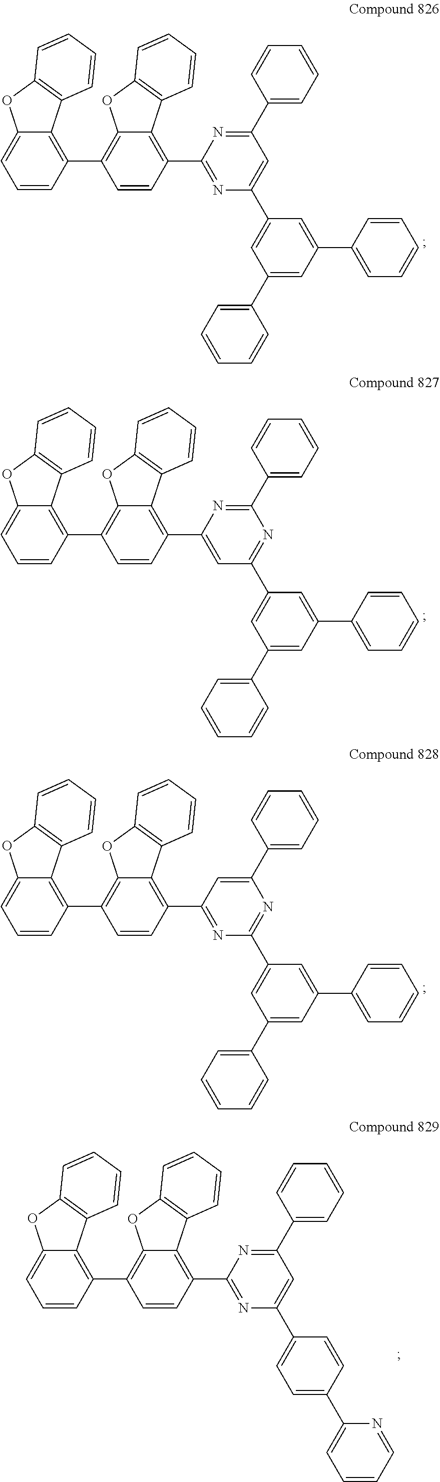 [SMILES: c1ccc(-c2cc(-c3ccccc3)cc(-c3cc(-c4ccccc4)nc(-c4ccc(-c5cccc6oc7ccccc7c56)c5oc6ccccc6c45)n3)c2)cc1 ; c1ccc(-c2cc(-c3ccccc3)cc(-c3cc(-c4ccc(-c5cccc6oc7ccccc7c56)c5oc6ccccc6c45)nc(-c4ccccc4)n3)c2)cc1 ; c1ccc(-c2cc(-c3ccccc3)cc(-c3nc(-c4ccccc4)cc(-c4ccc(-c5cccc6oc7ccccc7c56)c5oc6ccccc6c45)n3)c2)cc1 ; c1ccc(-c2cc(-c3ccc(-c4ccccn4)cc3)nc(-c3ccc(-c4cccc5oc6ccccc6c45)c4oc5ccccc5c34)n2)cc1]

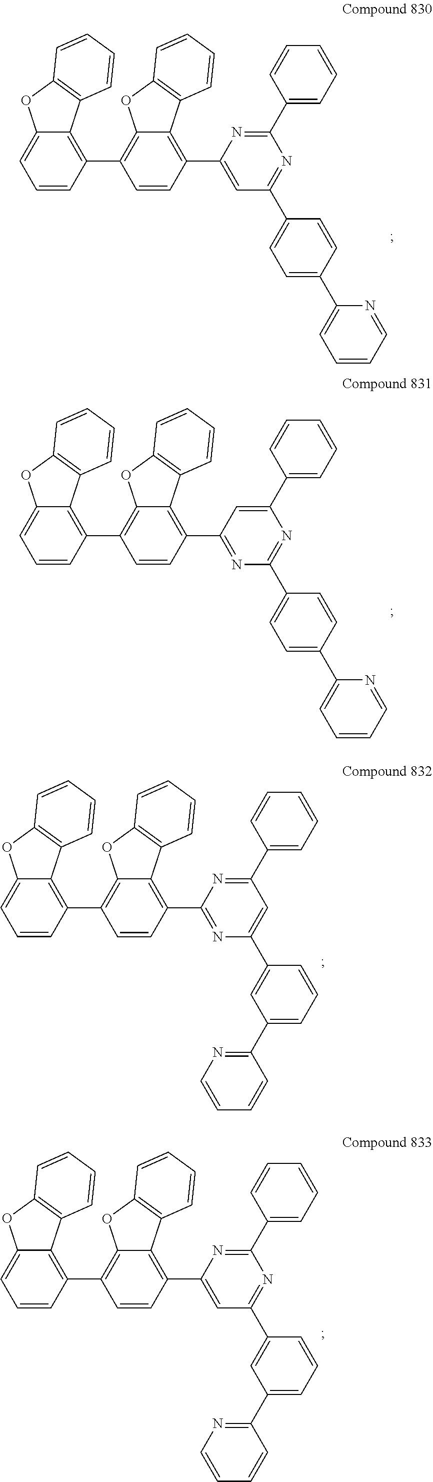 [SMILES: c1ccc(-c2nc(-c3ccc(-c4ccccn4)cc3)cc(-c3ccc(-c4cccc5oc6ccccc6c45)c4oc5ccccc5c34)n2)cc1 ; c1ccc(-c2cc(-c3ccc(-c4cccc5oc6ccccc6c45)c4oc5ccccc5c34)nc(-c3ccc(-c4ccccn4)cc3)n2)cc1 ; c1ccc(-c2cc(-c3cccc(-c4ccccn4)c3)nc(-c3ccc(-c4cccc5oc6ccccc6c45)c4oc5ccccc5c34)n2)cc1 ; c1ccc(-c2nc(-c3cccc(-c4ccccn4)c3)cc(-c3ccc(-c4cccc5oc6ccccc6c45)c4oc5ccccc5c34)n2)cc1]

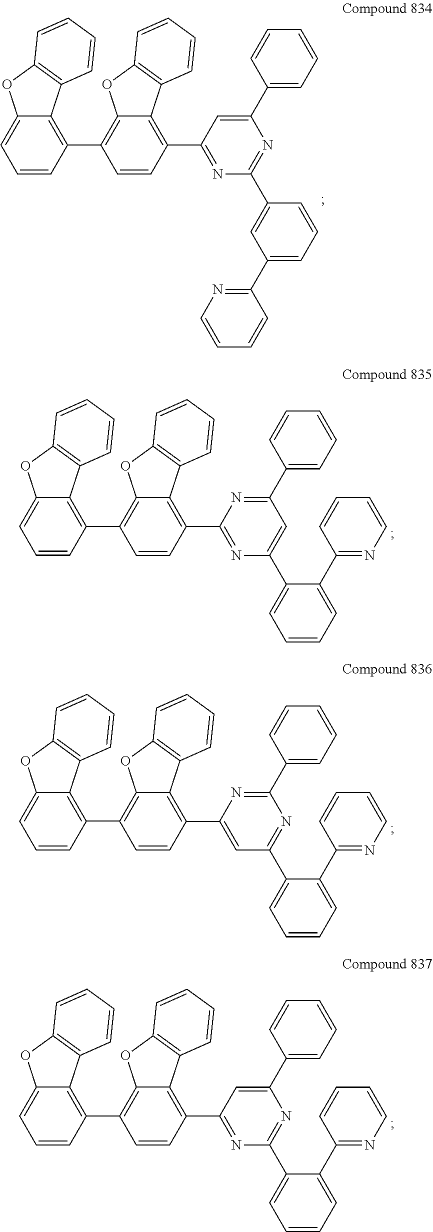 [SMILES: c1ccc(-c2cc(-c3ccc(-c4cccc5oc6ccccc6c45)c4oc5ccccc5c34)nc(-c3cccc(-c4ccccn4)c3)n2)cc1 ; c1ccc(-c2cc(-c3ccccc3-c3ccccn3)nc(-c3ccc(-c4cccc5oc6ccccc6c45)c4oc5ccccc5c34)n2)cc1 ; c1ccc(-c2nc(-c3ccccc3-c3ccccn3)cc(-c3ccc(-c4cccc5oc6ccccc6c45)c4oc5ccccc5c34)n2)cc1 ; c1ccc(-c2cc(-c3ccc(-c4cccc5oc6ccccc6c45)c4oc5ccccc5c34)nc(-c3ccccc3-c3ccccn3)n2)cc1]

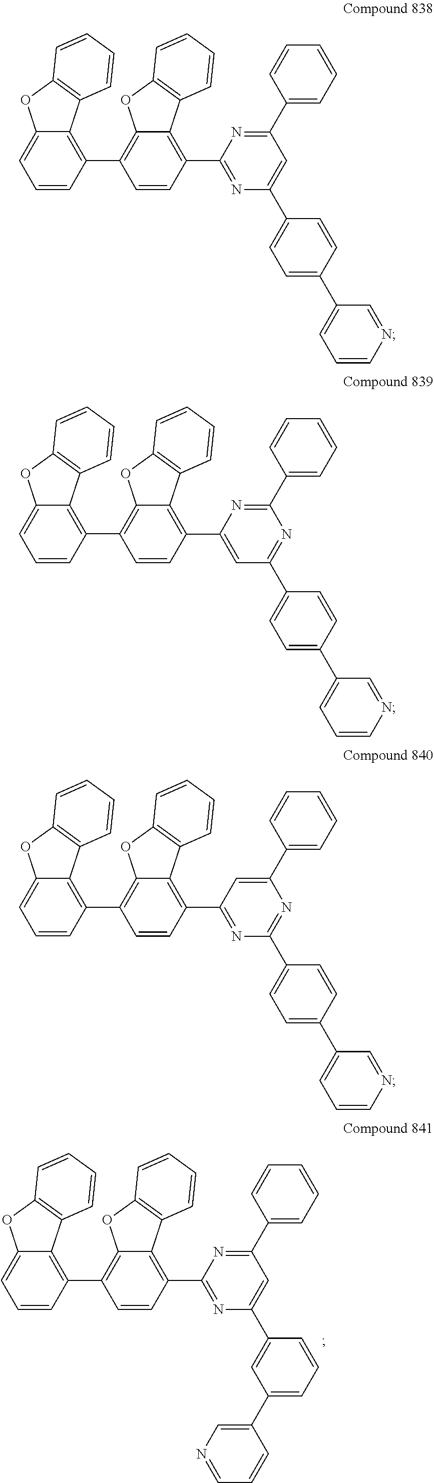 [SMILES: c1ccc(-c2cc(-c3ccc(-c4cccnc4)cc3)nc(-c3ccc(-c4cccc5oc6ccccc6c45)c4oc5ccccc5c34)n2)cc1 ; c1ccc(-c2nc(-c3ccc(-c4cccnc4)cc3)cc(-c3ccc(-c4cccc5oc6ccccc6c45)c4oc5ccccc5c34)n2)cc1 ; c1ccc(-c2cc(-c3ccc(-c4cccc5oc6ccccc6c45)c4oc5ccccc5c34)nc(-c3ccc(-c4cccnc4)cc3)n2)cc1 ; c1ccc(-c2cc(-c3cccc(-c4cccnc4)c3)nc(-c3ccc(-c4cccc5oc6ccccc6c45)c4oc5ccccc5c34)n2)cc1]

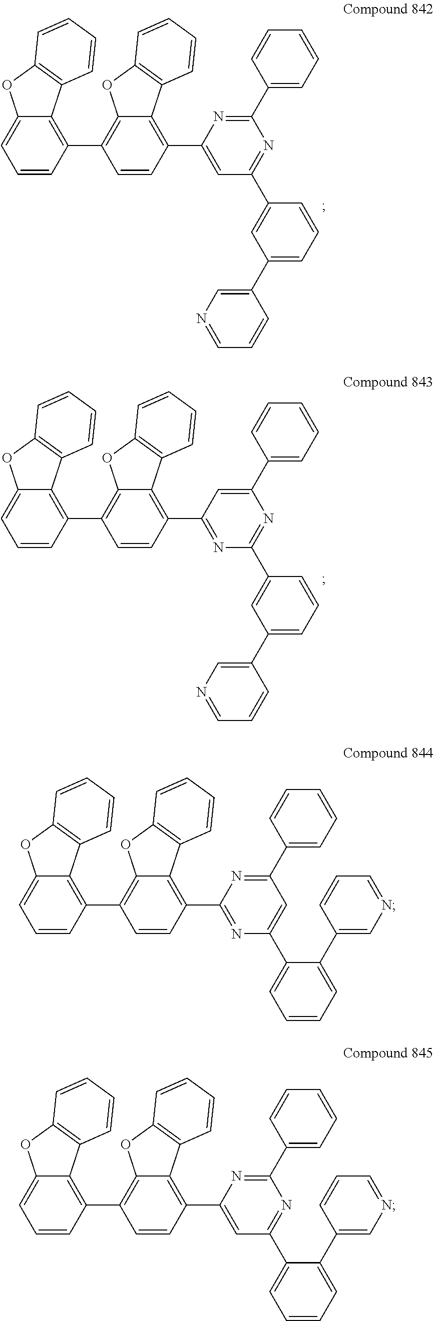 [SMILES: c1ccc(-c2nc(-c3cccc(-c4cccnc4)c3)cc(-c3ccc(-c4cccc5oc6ccccc6c45)c4oc5ccccc5c34)n2)cc1 ; c1ccc(-c2cc(-c3ccc(-c4cccc5oc6ccccc6c45)c4oc5ccccc5c34)nc(-c3cccc(-c4cccnc4)c3)n2)cc1 ; c1ccc(-c2cc(-c3ccccc3-c3cccnc3)nc(-c3ccc(-c4cccc5oc6ccccc6c45)c4oc5ccccc5c34)n2)cc1 ; c1ccc(-c2nc(-c3ccccc3-c3cccnc3)cc(-c3ccc(-c4cccc5oc6ccccc6c45)c4oc5ccccc5c34)n2)cc1]

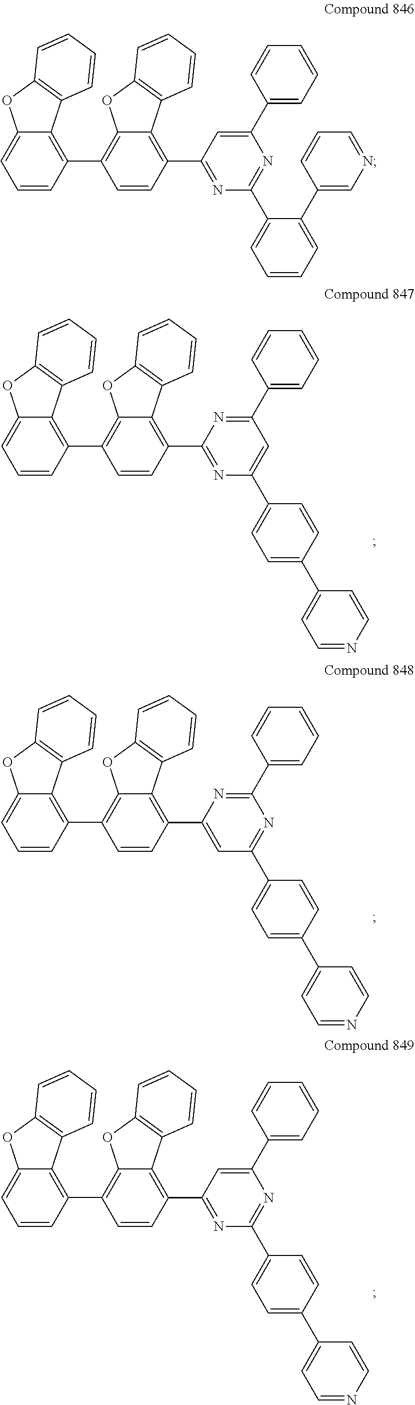 [SMILES: c1ccc(-c2cc(-c3ccc(-c4cccc5oc6ccccc6c45)c4oc5ccccc5c34)nc(-c3ccccc3-c3cccnc3)n2)cc1 ; c1ccc(-c2cc(-c3ccc(-c4ccncc4)cc3)nc(-c3ccc(-c4cccc5oc6ccccc6c45)c4oc5ccccc5c34)n2)cc1 ; c1ccc(-c2nc(-c3ccc(-c4ccncc4)cc3)cc(-c3ccc(-c4cccc5oc6ccccc6c45)c4oc5ccccc5c34)n2)cc1 ; c1ccc(-c2cc(-c3ccc(-c4cccc5oc6ccccc6c45)c4oc5ccccc5c34)nc(-c3ccc(-c4ccncc4)cc3)n2)cc1]

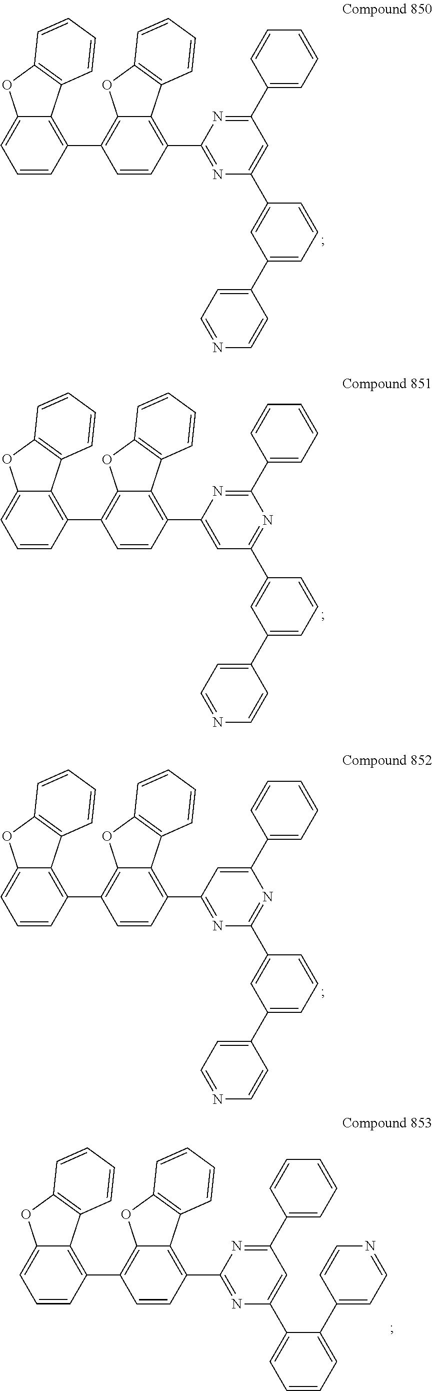 [SMILES: c1ccc(-c2cc(-c3cccc(-c4ccncc4)c3)nc(-c3ccc(-c4cccc5oc6ccccc6c45)c4oc5ccccc5c34)n2)cc1 ; c1ccc(-c2nc(-c3cccc(-c4ccncc4)c3)cc(-c3ccc(-c4cccc5oc6ccccc6c45)c4oc5ccccc5c34)n2)cc1 ; c1ccc(-c2cc(-c3ccc(-c4cccc5oc6ccccc6c45)c4oc5ccccc5c34)nc(-c3cccc(-c4ccncc4)c3)n2)cc1 ; c1ccc(-c2cc(-c3ccccc3-c3ccncc3)nc(-c3ccc(-c4cccc5oc6ccccc6c45)c4oc5ccccc5c34)n2)cc1]

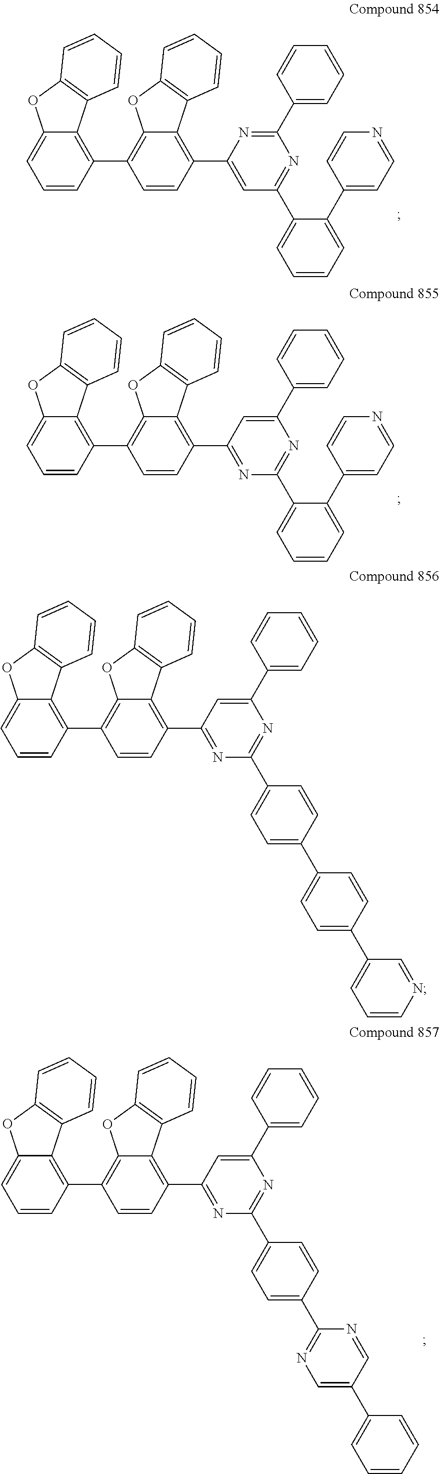 [SMILES: c1ccc(-c2nc(-c3ccccc3-c3ccncc3)cc(-c3ccc(-c4cccc5oc6ccccc6c45)c4oc5ccccc5c34)n2)cc1 ; c1ccc(-c2cc(-c3ccc(-c4cccc5oc6ccccc6c45)c4oc5ccccc5c34)nc(-c3ccccc3-c3ccncc3)n2)cc1 ; c1ccc(-c2cc(-c3ccc(-c4cccc5oc6ccccc6c45)c4oc5ccccc5c34)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6cccc7oc8ccccc8c67)c6oc7ccccc7c56)n4)cc3)nc2)cc1]

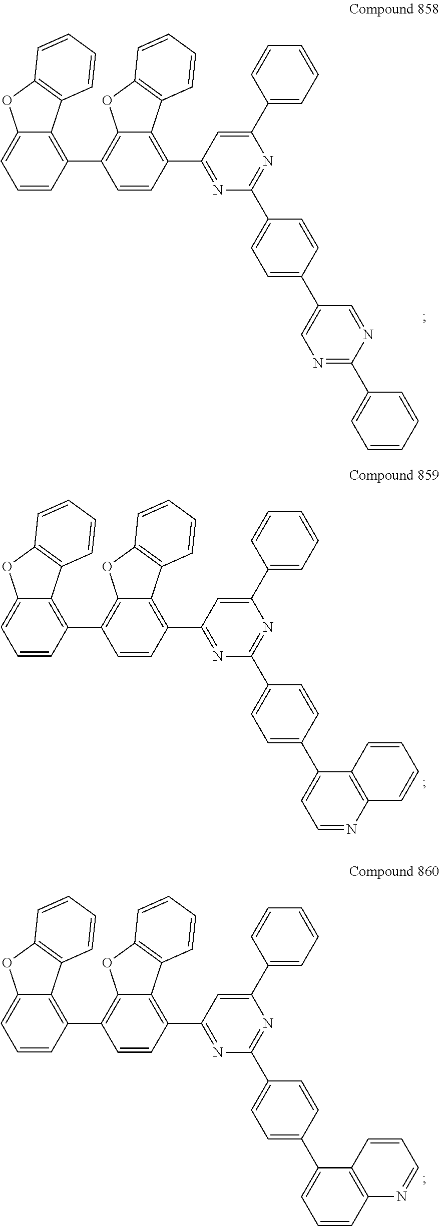 [SMILES: c1ccc(-c2cc(-c3ccc(-c4cccc5oc6ccccc6c45)c4oc5ccccc5c34)nc(-c3ccc(-c4cnc(-c5ccccc5)nc4)cc3)n2)cc1 ; c1ccc(-c2cc(-c3ccc(-c4cccc5oc6ccccc6c45)c4oc5ccccc5c34)nc(-c3ccc(-c4ccnc5ccccc45)cc3)n2)cc1 ; c1ccc(-c2cc(-c3ccc(-c4cccc5oc6ccccc6c45)c4oc5ccccc5c34)nc(-c3ccc(-c4cccc5ncccc45)cc3)n2)cc1]

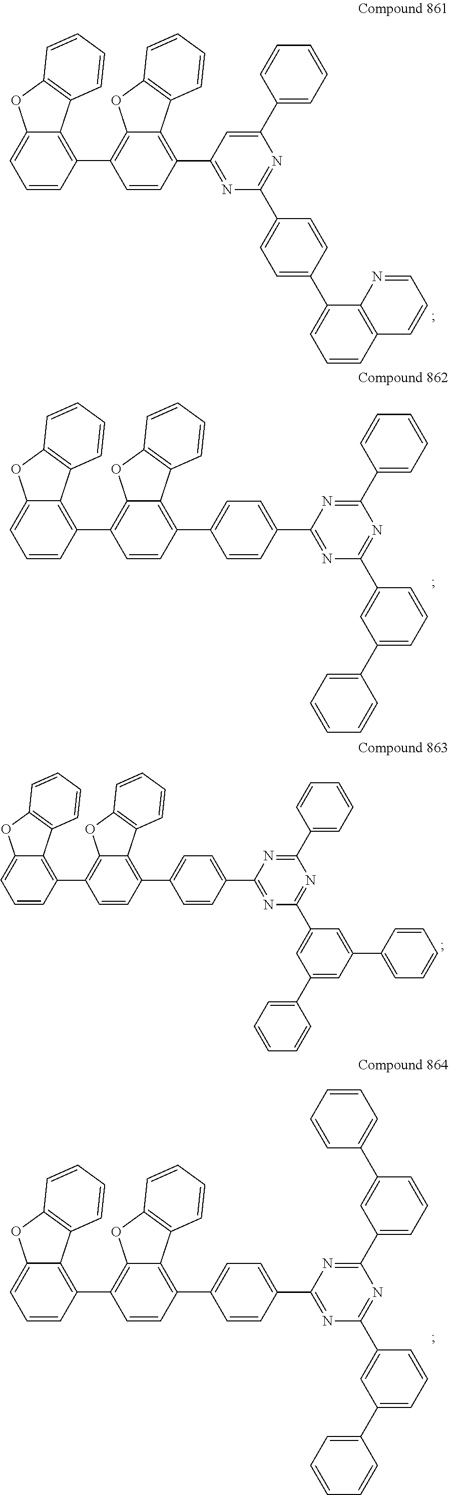 [SMILES: c1ccc(-c2cc(-c3ccc(-c4cccc5oc6ccccc6c45)c4oc5ccccc5c34)nc(-c3ccc(-c4cccc5cccnc45)cc3)n2)cc1 ; c1ccc(-c2cccc(-c3nc(-c4ccccc4)nc(-c4ccc(-c5ccc(-c6cccc7oc8ccccc8c67)c6oc7ccccc7c56)cc4)n3)c2)cc1 ; c1ccc(-c2cc(-c3ccccc3)cc(-c3nc(-c4ccccc4)nc(-c4ccc(-c5ccc(-c6cccc7oc8ccccc8c67)c6oc7ccccc7c56)cc4)n3)c2)cc1 ; c1ccc(-c2cccc(-c3nc(-c4ccc(-c5ccc(-c6cccc7oc8ccccc8c67)c6oc7ccccc7c56)cc4)nc(-c4cccc(-c5ccccc5)c4)n3)c2)cc1]

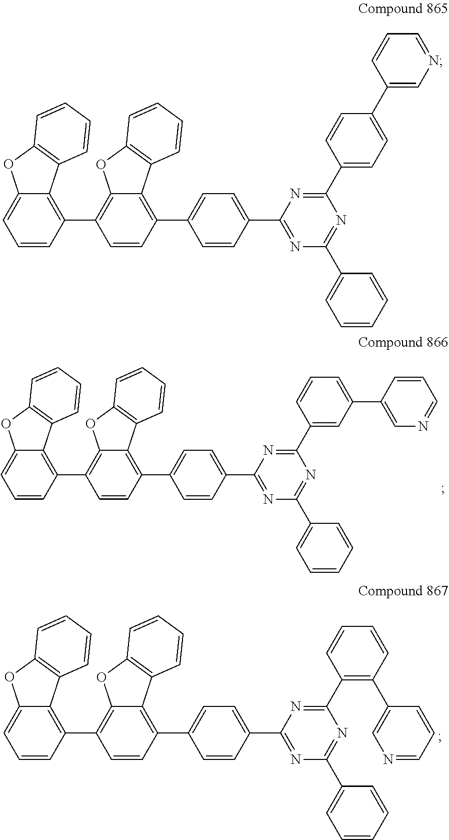 [SMILES: c1ccc(-c2nc(-c3ccc(-c4cccnc4)cc3)nc(-c3ccc(-c4ccc(-c5cccc6oc7ccccc7c56)c5oc6ccccc6c45)cc3)n2)cc1 ; c1ccc(-c2nc(-c3ccc(-c4ccc(-c5cccc6oc7ccccc7c56)c5oc6ccccc6c45)cc3)nc(-c3cccc(-c4cccnc4)c3)n2)cc1 ; c1ccc(-c2nc(-c3ccc(-c4ccc(-c5cccc6oc7ccccc7c56)c5oc6ccccc6c45)cc3)nc(-c3ccccc3-c3cccnc3)n2)cc1]

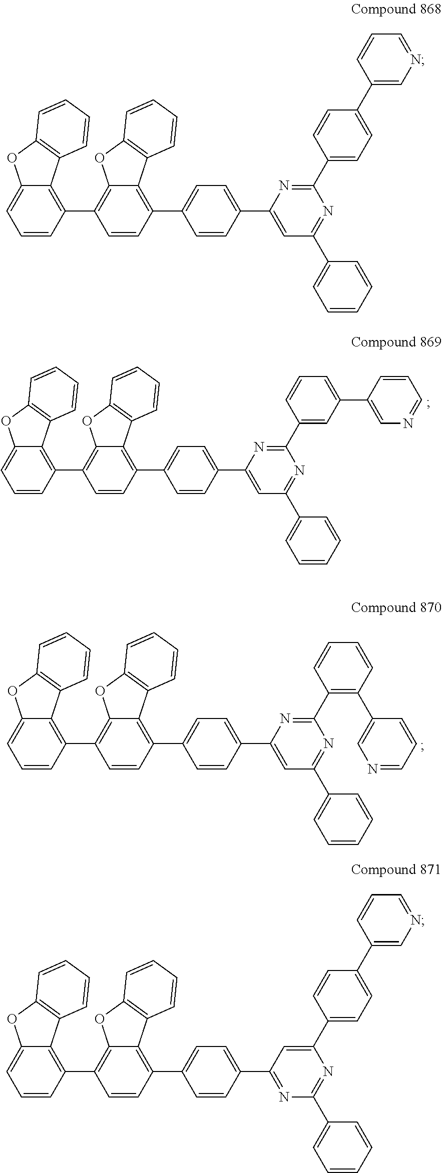 [SMILES: c1ccc(-c2cc(-c3ccc(-c4ccc(-c5cccc6oc7ccccc7c56)c5oc6ccccc6c45)cc3)nc(-c3ccc(-c4cccnc4)cc3)n2)cc1 ; c1ccc(-c2cc(-c3ccc(-c4ccc(-c5cccc6oc7ccccc7c56)c5oc6ccccc6c45)cc3)nc(-c3cccc(-c4cccnc4)c3)n2)cc1 ; c1ccc(-c2cc(-c3ccc(-c4ccc(-c5cccc6oc7ccccc7c56)c5oc6ccccc6c45)cc3)nc(-c3ccccc3-c3cccnc3)n2)cc1 ; c1ccc(-c2nc(-c3ccc(-c4cccnc4)cc3)cc(-c3ccc(-c4ccc(-c5cccc6oc7ccccc7c56)c5oc6ccccc6c45)cc3)n2)cc1]

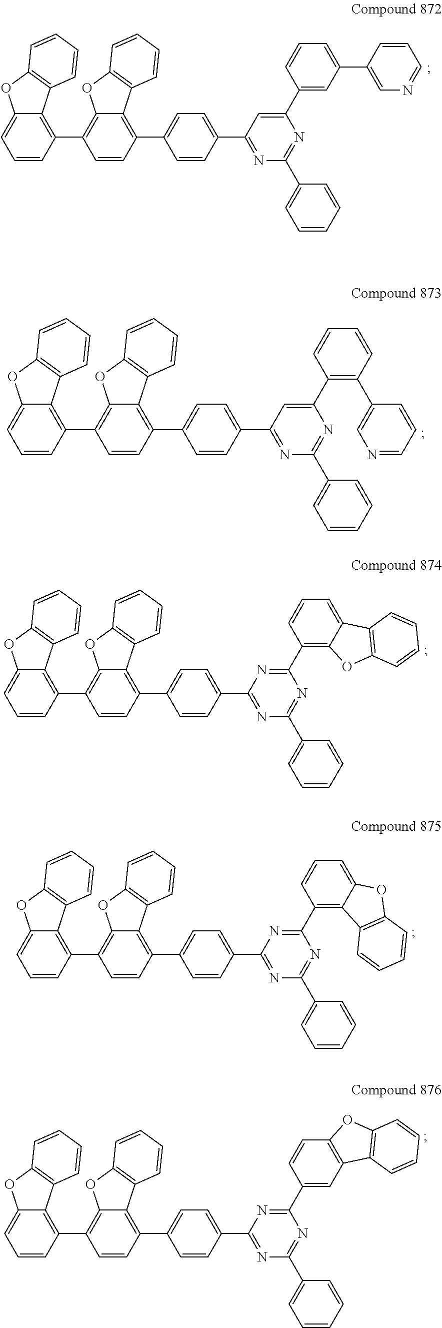 [SMILES: c1ccc(-c2nc(-c3ccc(-c4ccc(-c5cccc6oc7ccccc7c56)c5oc6ccccc6c45)cc3)cc(-c3cccc(-c4cccnc4)c3)n2)cc1 ; c1ccc(-c2nc(-c3ccc(-c4ccc(-c5cccc6oc7ccccc7c56)c5oc6ccccc6c45)cc3)cc(-c3ccccc3-c3cccnc3)n2)cc1 ; c1ccc(-c2nc(-c3ccc(-c4ccc(-c5cccc6oc7ccccc7c56)c5oc6ccccc6c45)cc3)nc(-c3cccc4c3oc3ccccc34)n2)cc1 ; c1ccc(-c2nc(-c3ccc(-c4ccc(-c5cccc6oc7ccccc7c56)c5oc6ccccc6c45)cc3)nc(-c3cccc4oc5ccccc5c34)n2)cc1 ; c1ccc(-c2nc(-c3ccc(-c4ccc(-c5cccc6oc7ccccc7c56)c5oc6ccccc6c45)cc3)nc(-c3ccc4oc5ccccc5c4c3)n2)cc1]

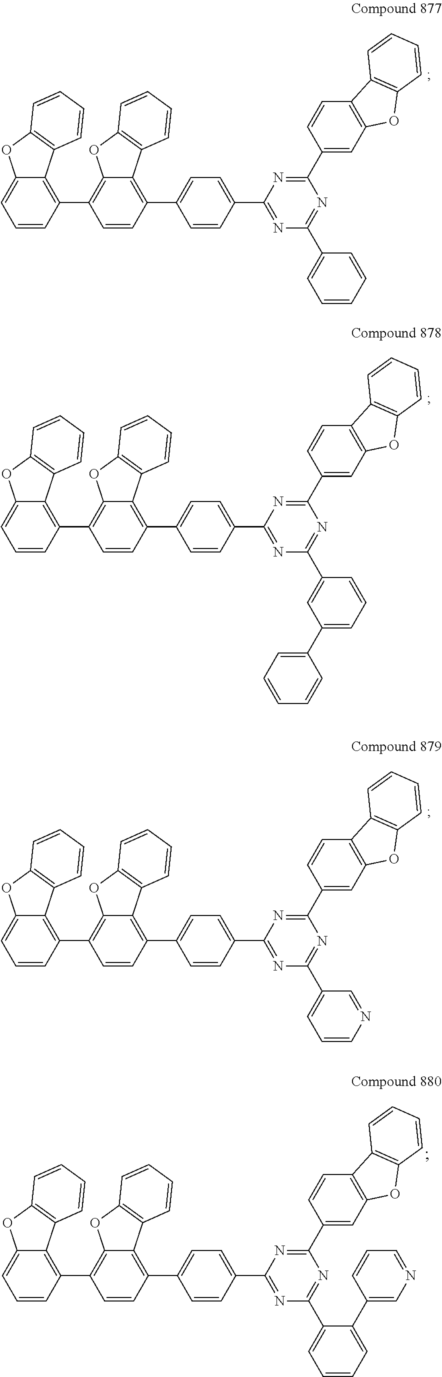 [SMILES: c1ccc(-c2nc(-c3ccc(-c4ccc(-c5cccc6oc7ccccc7c56)c5oc6ccccc6c45)cc3)nc(-c3ccc4c(c3)oc3ccccc34)n2)cc1 ; c1ccc(-c2cccc(-c3nc(-c4ccc(-c5ccc(-c6cccc7oc8ccccc8c67)c6oc7ccccc7c56)cc4)nc(-c4ccc5c(c4)oc4ccccc45)n3)c2)cc1 ; c1cncc(-c2nc(-c3ccc(-c4ccc(-c5cccc6oc7ccccc7c56)c5oc6ccccc6c45)cc3)nc(-c3ccc4c(c3)oc3ccccc34)n2)c1 ; c1cncc(-c2ccccc2-c2nc(-c3ccc(-c4ccc(-c5cccc6oc7ccccc7c56)c5oc6ccccc6c45)cc3)nc(-c3ccc4c(c3)oc3ccccc34)n2)c1]

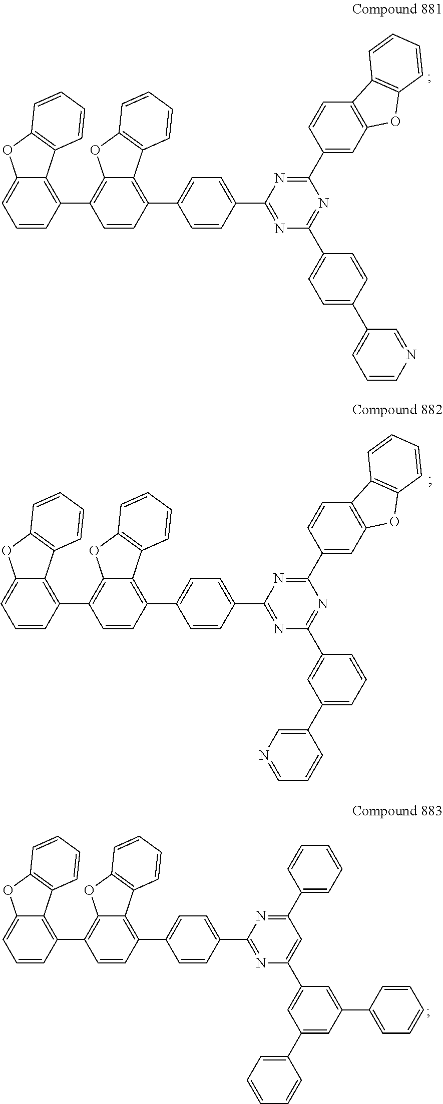 [SMILES: c1cncc(-c2ccc(-c3nc(-c4ccc(-c5ccc(-c6cccc7oc8ccccc8c67)c6oc7ccccc7c56)cc4)nc(-c4ccc5c(c4)oc4ccccc45)n3)cc2)c1 ; c1cncc(-c2cccc(-c3nc(-c4ccc(-c5ccc(-c6cccc7oc8ccccc8c67)c6oc7ccccc7c56)cc4)nc(-c4ccc5c(c4)oc4ccccc45)n3)c2)c1 ; c1ccc(-c2cc(-c3ccccc3)cc(-c3cc(-c4ccccc4)nc(-c4ccc(-c5ccc(-c6cccc7oc8ccccc8c67)c6oc7ccccc7c56)cc4)n3)c2)cc1]

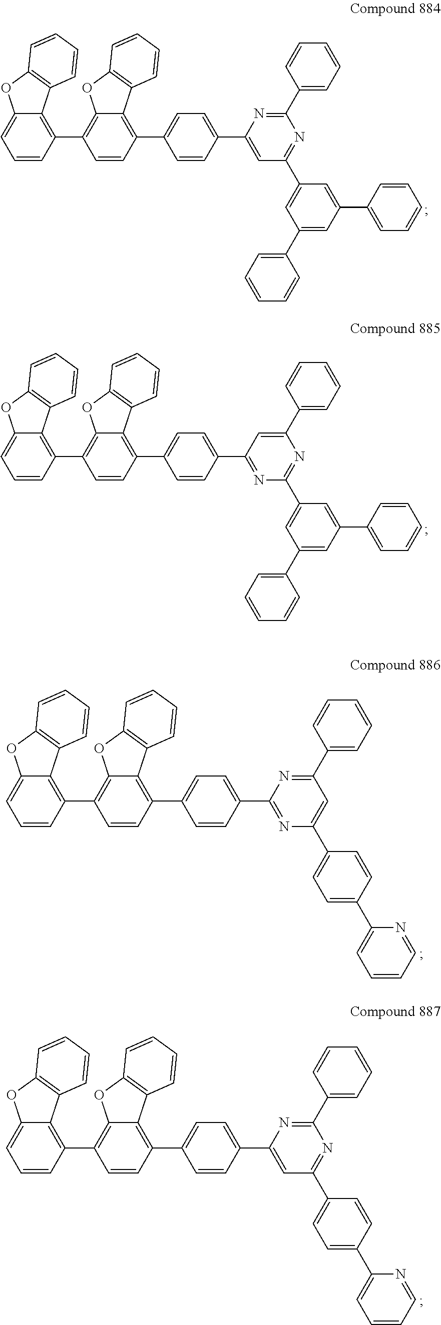 [SMILES: c1ccc(-c2cc(-c3ccccc3)cc(-c3cc(-c4ccc(-c5ccc(-c6cccc7oc8ccccc8c67)c6oc7ccccc7c56)cc4)nc(-c4ccccc4)n3)c2)cc1 ; c1ccc(-c2cc(-c3ccccc3)cc(-c3nc(-c4ccccc4)cc(-c4ccc(-c5ccc(-c6cccc7oc8ccccc8c67)c6oc7ccccc7c56)cc4)n3)c2)cc1 ; c1ccc(-c2cc(-c3ccc(-c4ccccn4)cc3)nc(-c3ccc(-c4ccc(-c5cccc6oc7ccccc7c56)c5oc6ccccc6c45)cc3)n2)cc1 ; c1ccc(-c2nc(-c3ccc(-c4ccccn4)cc3)cc(-c3ccc(-c4ccc(-c5cccc6oc7ccccc7c56)c5oc6ccccc6c45)cc3)n2)cc1]

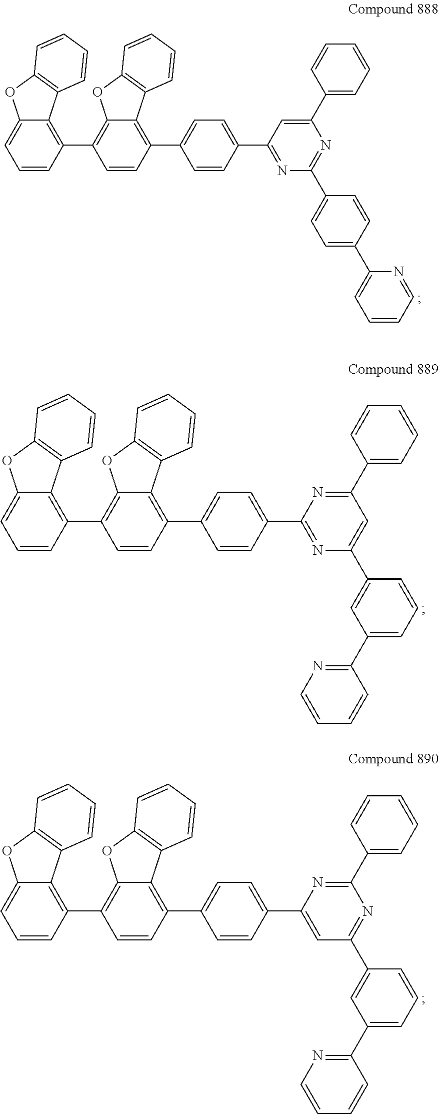 [SMILES: c1ccc(-c2cc(-c3ccc(-c4ccc(-c5cccc6oc7ccccc7c56)c5oc6ccccc6c45)cc3)nc(-c3ccc(-c4ccccn4)cc3)n2)cc1 ; c1ccc(-c2cc(-c3cccc(-c4ccccn4)c3)nc(-c3ccc(-c4ccc(-c5cccc6oc7ccccc7c56)c5oc6ccccc6c45)cc3)n2)cc1 ; c1ccc(-c2nc(-c3ccc(-c4ccc(-c5cccc6oc7ccccc7c56)c5oc6ccccc6c45)cc3)cc(-c3cccc(-c4ccccn4)c3)n2)cc1]

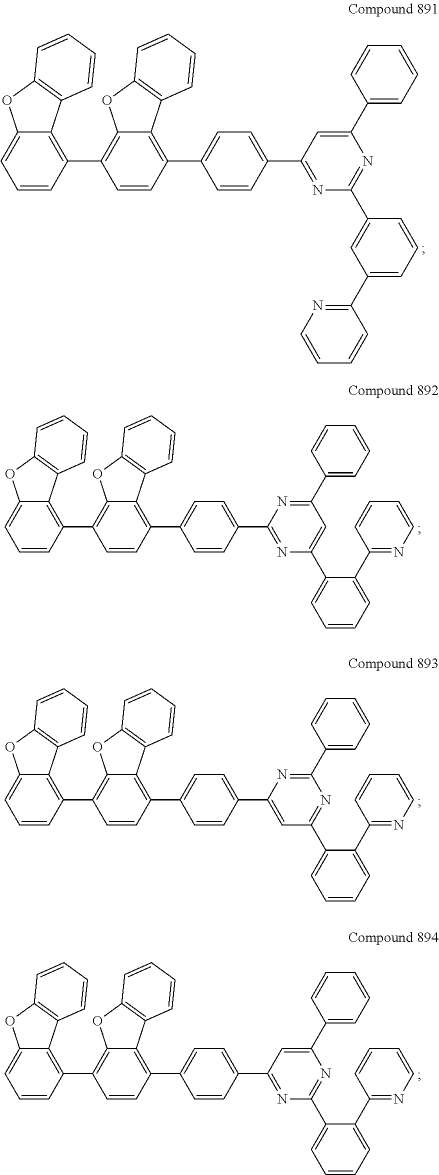 [SMILES: c1ccc(-c2cc(-c3ccc(-c4ccc(-c5cccc6oc7ccccc7c56)c5oc6ccccc6c45)cc3)nc(-c3cccc(-c4ccccn4)c3)n2)cc1 ; c1ccc(-c2cc(-c3ccccc3-c3ccccn3)nc(-c3ccc(-c4ccc(-c5cccc6oc7ccccc7c56)c5oc6ccccc6c45)cc3)n2)cc1 ; c1ccc(-c2nc(-c3ccc(-c4ccc(-c5cccc6oc7ccccc7c56)c5oc6ccccc6c45)cc3)cc(-c3ccccc3-c3ccccn3)n2)cc1 ; c1ccc(-c2cc(-c3ccc(-c4ccc(-c5cccc6oc7ccccc7c56)c5oc6ccccc6c45)cc3)nc(-c3ccccc3-c3ccccn3)n2)cc1]

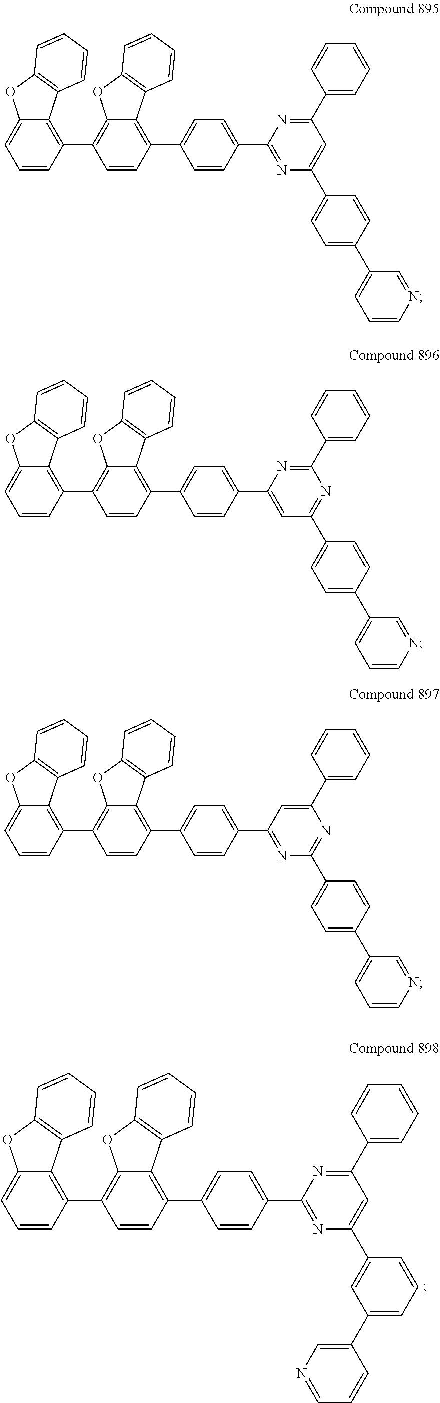 [SMILES: c1ccc(-c2cc(-c3ccc(-c4cccnc4)cc3)nc(-c3ccc(-c4ccc(-c5cccc6oc7ccccc7c56)c5oc6ccccc6c45)cc3)n2)cc1 ; c1ccc(-c2nc(-c3ccc(-c4cccnc4)cc3)cc(-c3ccc(-c4ccc(-c5cccc6oc7ccccc7c56)c5oc6ccccc6c45)cc3)n2)cc1 ; c1ccc(-c2cc(-c3ccc(-c4ccc(-c5cccc6oc7ccccc7c56)c5oc6ccccc6c45)cc3)nc(-c3ccc(-c4cccnc4)cc3)n2)cc1 ; c1ccc(-c2cc(-c3cccc(-c4cccnc4)c3)nc(-c3ccc(-c4ccc(-c5cccc6oc7ccccc7c56)c5oc6ccccc6c45)cc3)n2)cc1]

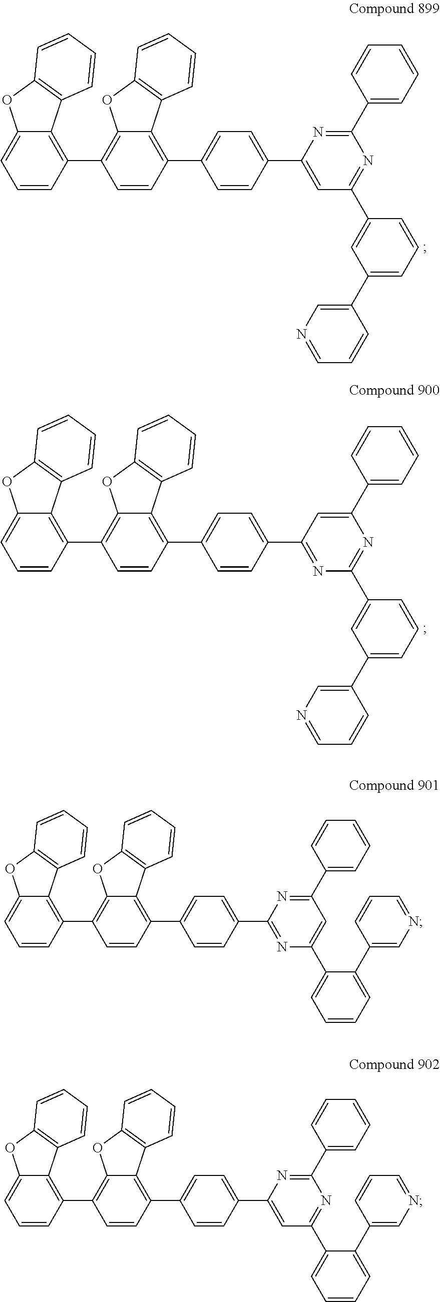 [SMILES: c1ccc(-c2nc(-c3ccc(-c4ccc(-c5cccc6oc7ccccc7c56)c5oc6ccccc6c45)cc3)cc(-c3cccc(-c4cccnc4)c3)n2)cc1 ; c1ccc(-c2cc(-c3ccc(-c4ccc(-c5cccc6oc7ccccc7c56)c5oc6ccccc6c45)cc3)nc(-c3cccc(-c4cccnc4)c3)n2)cc1 ; c1ccc(-c2cc(-c3ccccc3-c3cccnc3)nc(-c3ccc(-c4ccc(-c5cccc6oc7ccccc7c56)c5oc6ccccc6c45)cc3)n2)cc1 ; c1ccc(-c2nc(-c3ccc(-c4ccc(-c5cccc6oc7ccccc7c56)c5oc6ccccc6c45)cc3)cc(-c3ccccc3-c3cccnc3)n2)cc1]

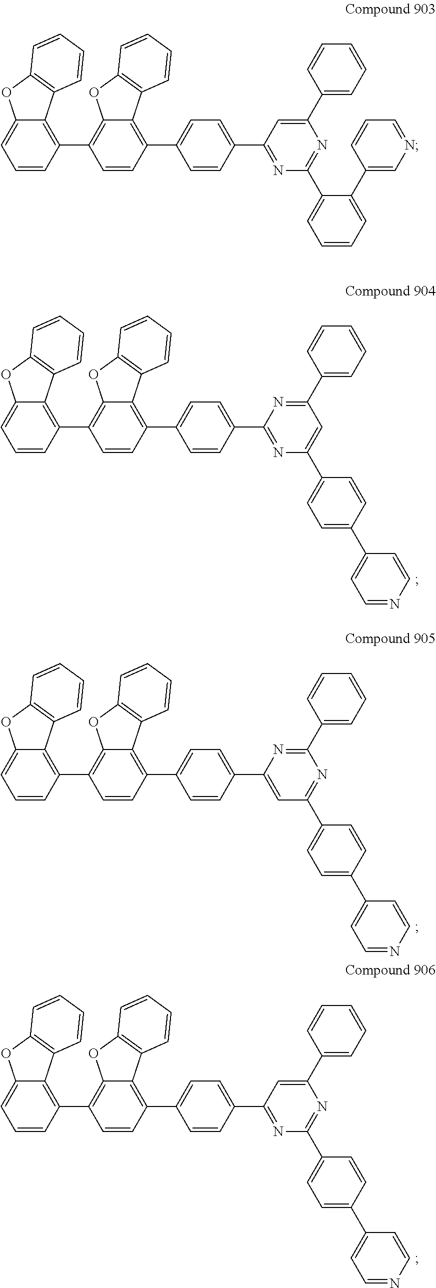 [SMILES: c1ccc(-c2cc(-c3ccc(-c4ccc(-c5cccc6oc7ccccc7c56)c5oc6ccccc6c45)cc3)nc(-c3ccccc3-c3cccnc3)n2)cc1 ; c1ccc(-c2cc(-c3ccc(-c4ccncc4)cc3)nc(-c3ccc(-c4ccc(-c5cccc6oc7ccccc7c56)c5oc6ccccc6c45)cc3)n2)cc1 ; c1ccc(-c2nc(-c3ccc(-c4ccncc4)cc3)cc(-c3ccc(-c4ccc(-c5cccc6oc7ccccc7c56)c5oc6ccccc6c45)cc3)n2)cc1 ; c1ccc(-c2cc(-c3ccc(-c4ccc(-c5cccc6oc7ccccc7c56)c5oc6ccccc6c45)cc3)nc(-c3ccc(-c4ccncc4)cc3)n2)cc1]

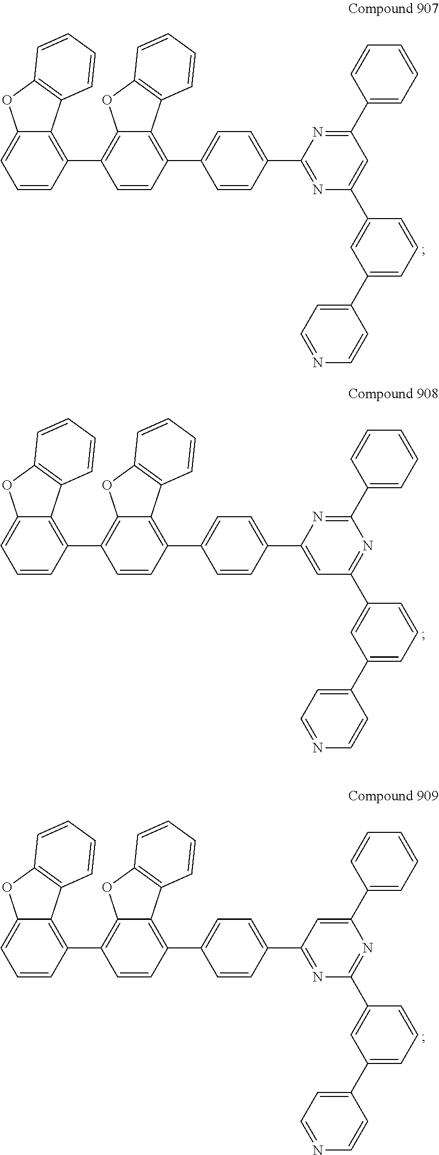 [SMILES: c1ccc(-c2cc(-c3cccc(-c4ccncc4)c3)nc(-c3ccc(-c4ccc(-c5cccc6oc7ccccc7c56)c5oc6ccccc6c45)cc3)n2)cc1 ; c1ccc(-c2nc(-c3ccc(-c4ccc(-c5cccc6oc7ccccc7c56)c5oc6ccccc6c45)cc3)cc(-c3cccc(-c4ccncc4)c3)n2)cc1 ; c1ccc(-c2cc(-c3ccc(-c4ccc(-c5cccc6oc7ccccc7c56)c5oc6ccccc6c45)cc3)nc(-c3cccc(-c4ccncc4)c3)n2)cc1]

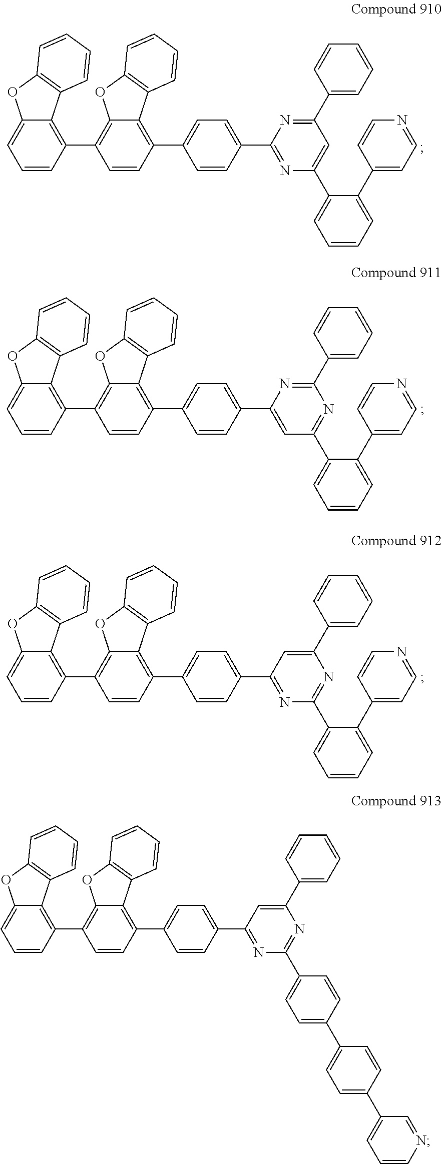 [SMILES: c1ccc(-c2cc(-c3ccccc3-c3ccncc3)nc(-c3ccc(-c4ccc(-c5cccc6oc7ccccc7c56)c5oc6ccccc6c45)cc3)n2)cc1 ; c1ccc(-c2nc(-c3ccc(-c4ccc(-c5cccc6oc7ccccc7c56)c5oc6ccccc6c45)cc3)cc(-c3ccccc3-c3ccncc3)n2)cc1 ; c1ccc(-c2cc(-c3ccc(-c4ccc(-c5cccc6oc7ccccc7c56)c5oc6ccccc6c45)cc3)nc(-c3ccccc3-c3ccncc3)n2)cc1 ; c1ccc(-c2cc(-c3ccc(-c4ccc(-c5cccc6oc7ccccc7c56)c5oc6ccccc6c45)cc3)nc(-c3ccc(-c4ccc(-c5cccnc5)cc4)cc3)n2)cc1]

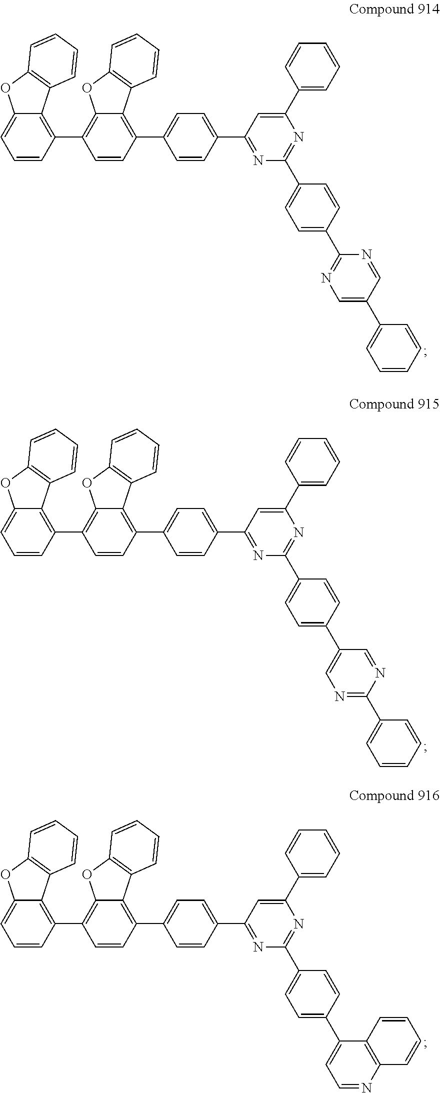 [SMILES: c1ccc(-c2cnc(-c3ccc(-c4nc(-c5ccccc5)cc(-c5ccc(-c6ccc(-c7cccc8oc9ccccc9c78)c7oc8ccccc8c67)cc5)n4)cc3)nc2)cc1 ; c1ccc(-c2cc(-c3ccc(-c4ccc(-c5cccc6oc7ccccc7c56)c5oc6ccccc6c45)cc3)nc(-c3ccc(-c4cnc(-c5ccccc5)nc4)cc3)n2)cc1 ; c1ccc(-c2cc(-c3ccc(-c4ccc(-c5cccc6oc7ccccc7c56)c5oc6ccccc6c45)cc3)nc(-c3ccc(-c4ccnc5ccccc45)cc3)n2)cc1]

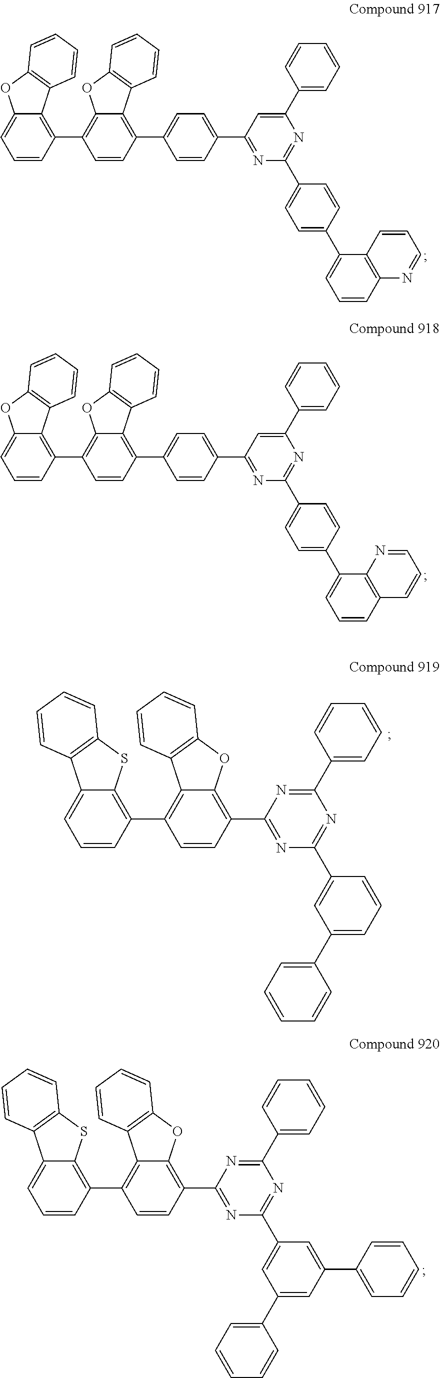 [SMILES: c1ccc(-c2cc(-c3ccc(-c4ccc(-c5cccc6oc7ccccc7c56)c5oc6ccccc6c45)cc3)nc(-c3ccc(-c4cccc5ncccc45)cc3)n2)cc1 ; c1ccc(-c2cc(-c3ccc(-c4ccc(-c5cccc6oc7ccccc7c56)c5oc6ccccc6c45)cc3)nc(-c3ccc(-c4cccc5cccnc45)cc3)n2)cc1 ; c1ccc(-c2cccc(-c3nc(-c4ccccc4)nc(-c4ccc(-c5cccc6c5sc5ccccc56)c5c4oc4ccccc45)n3)c2)cc1 ; c1ccc(-c2cc(-c3ccccc3)cc(-c3nc(-c4ccccc4)nc(-c4ccc(-c5cccc6c5sc5ccccc56)c5c4oc4ccccc45)n3)c2)cc1]

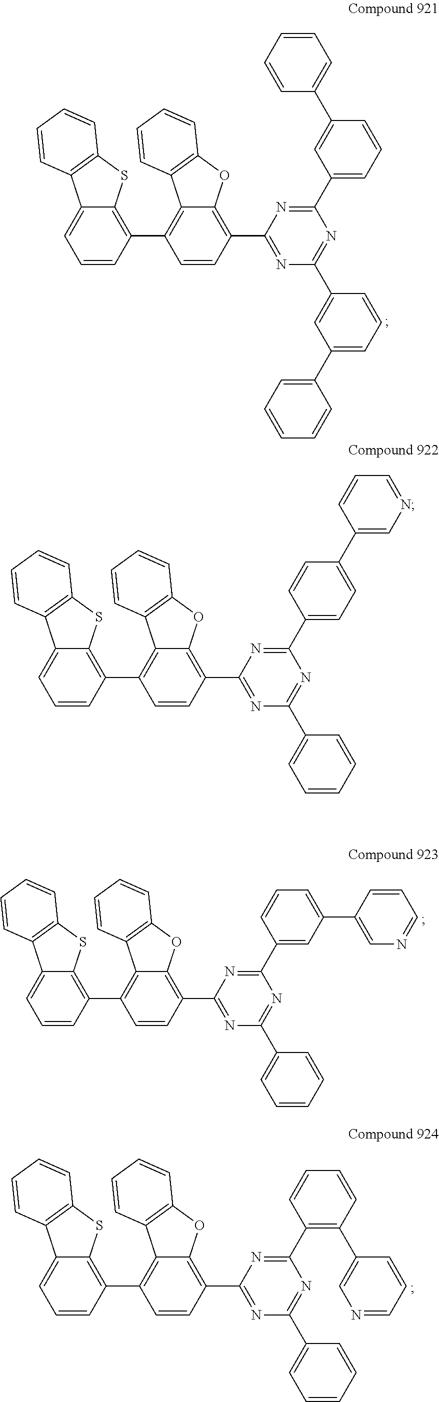 [SMILES: c1ccc(-c2cccc(-c3nc(-c4cccc(-c5ccccc5)c4)nc(-c4ccc(-c5cccc6c5sc5ccccc56)c5c4oc4ccccc45)n3)c2)cc1 ; c1ccc(-c2nc(-c3ccc(-c4cccnc4)cc3)nc(-c3ccc(-c4cccc5c4sc4ccccc45)c4c3oc3ccccc34)n2)cc1 ; c1ccc(-c2nc(-c3cccc(-c4cccnc4)c3)nc(-c3ccc(-c4cccc5c4sc4ccccc45)c4c3oc3ccccc34)n2)cc1 ; c1ccc(-c2nc(-c3ccccc3-c3cccnc3)nc(-c3ccc(-c4cccc5c4sc4ccccc45)c4c3oc3ccccc34)n2)cc1]

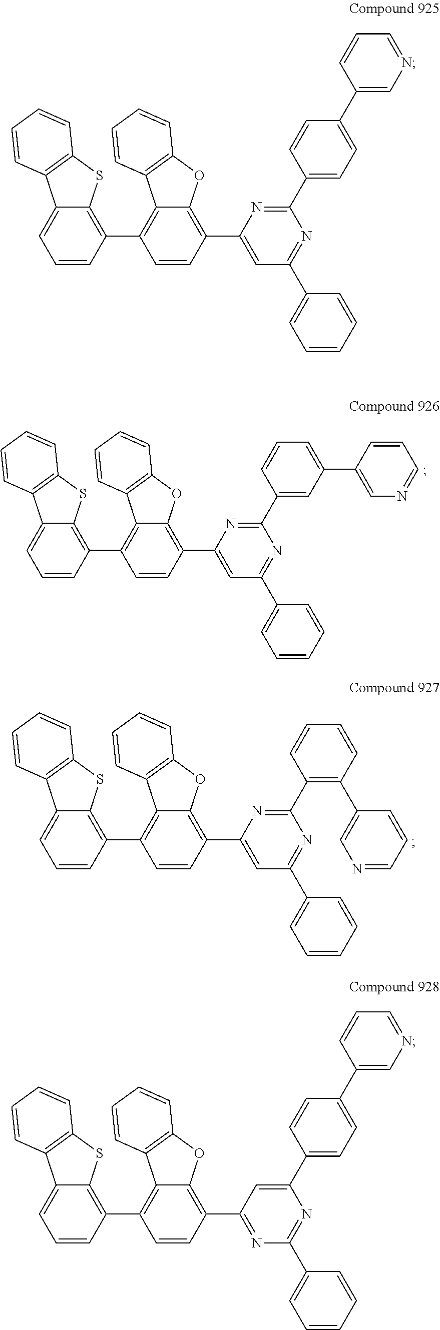 [SMILES: c1ccc(-c2cc(-c3ccc(-c4cccc5c4sc4ccccc45)c4c3oc3ccccc34)nc(-c3ccc(-c4cccnc4)cc3)n2)cc1 ; c1ccc(-c2cc(-c3ccc(-c4cccc5c4sc4ccccc45)c4c3oc3ccccc34)nc(-c3cccc(-c4cccnc4)c3)n2)cc1 ; c1ccc(-c2cc(-c3ccc(-c4cccc5c4sc4ccccc45)c4c3oc3ccccc34)nc(-c3ccccc3-c3cccnc3)n2)cc1 ; c1ccc(-c2nc(-c3ccc(-c4cccnc4)cc3)cc(-c3ccc(-c4cccc5c4sc4ccccc45)c4c3oc3ccccc34)n2)cc1]

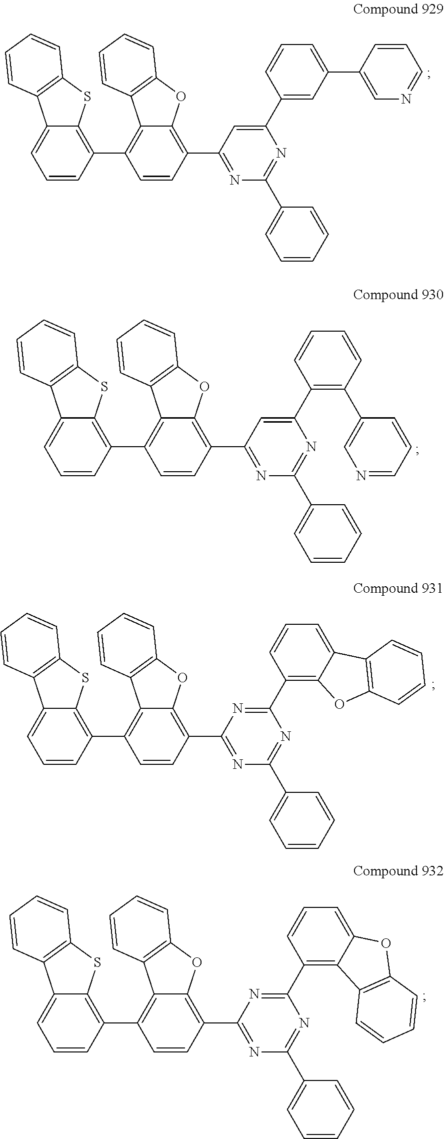 [SMILES: c1ccc(-c2nc(-c3cccc(-c4cccnc4)c3)cc(-c3ccc(-c4cccc5c4sc4ccccc45)c4c3oc3ccccc34)n2)cc1 ; c1ccc(-c2nc(-c3ccccc3-c3cccnc3)cc(-c3ccc(-c4cccc5c4sc4ccccc45)c4c3oc3ccccc34)n2)cc1 ; c1ccc(-c2nc(-c3cccc4c3oc3ccccc34)nc(-c3ccc(-c4cccc5c4sc4ccccc45)c4c3oc3ccccc34)n2)cc1 ; c1ccc(-c2nc(-c3ccc(-c4cccc5c4sc4ccccc45)c4c3oc3ccccc34)nc(-c3cccc4oc5ccccc5c34)n2)cc1]

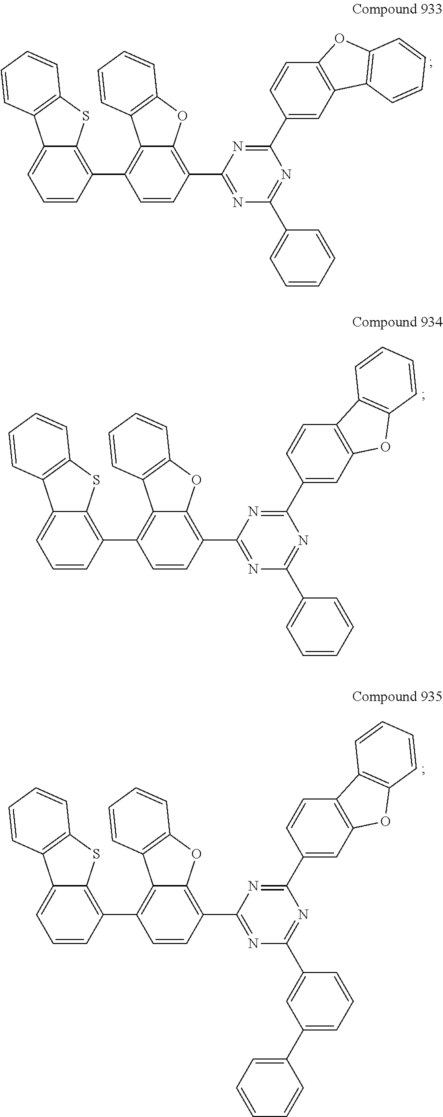 [SMILES: c1ccc(-c2nc(-c3ccc4oc5ccccc5c4c3)nc(-c3ccc(-c4cccc5c4sc4ccccc45)c4c3oc3ccccc34)n2)cc1 ; c1ccc(-c2nc(-c3ccc4c(c3)oc3ccccc34)nc(-c3ccc(-c4cccc5c4sc4ccccc45)c4c3oc3ccccc34)n2)cc1 ; c1ccc(-c2cccc(-c3nc(-c4ccc5c(c4)oc4ccccc45)nc(-c4ccc(-c5cccc6c5sc5ccccc56)c5c4oc4ccccc45)n3)c2)cc1]

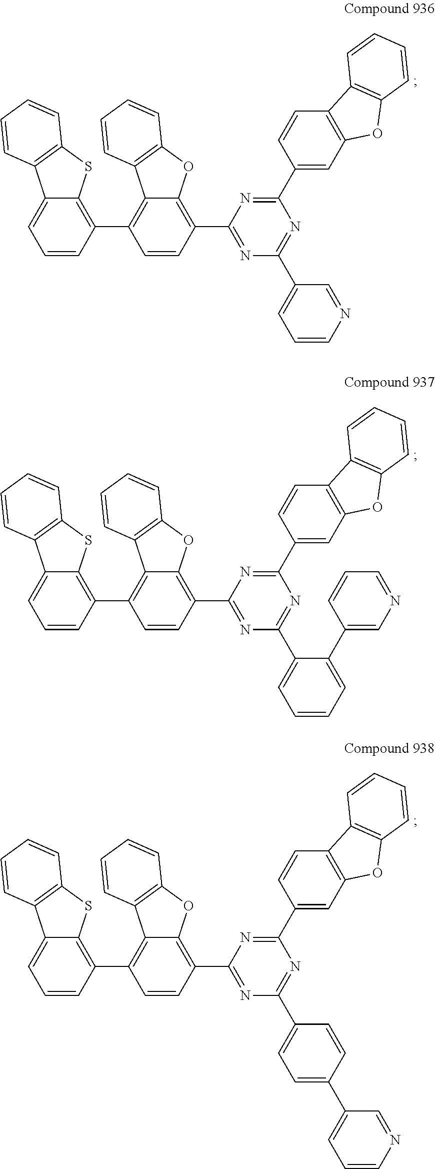 [SMILES: c1cncc(-c2nc(-c3ccc4c(c3)oc3ccccc34)nc(-c3ccc(-c4cccc5c4sc4ccccc45)c4c3oc3ccccc34)n2)c1 ; c1cncc(-c2ccccc2-c2nc(-c3ccc4c(c3)oc3ccccc34)nc(-c3ccc(-c4cccc5c4sc4ccccc45)c4c3oc3ccccc34)n2)c1 ; c1cncc(-c2ccc(-c3nc(-c4ccc5c(c4)oc4ccccc45)nc(-c4ccc(-c5cccc6c5sc5ccccc56)c5c4oc4ccccc45)n3)cc2)c1]

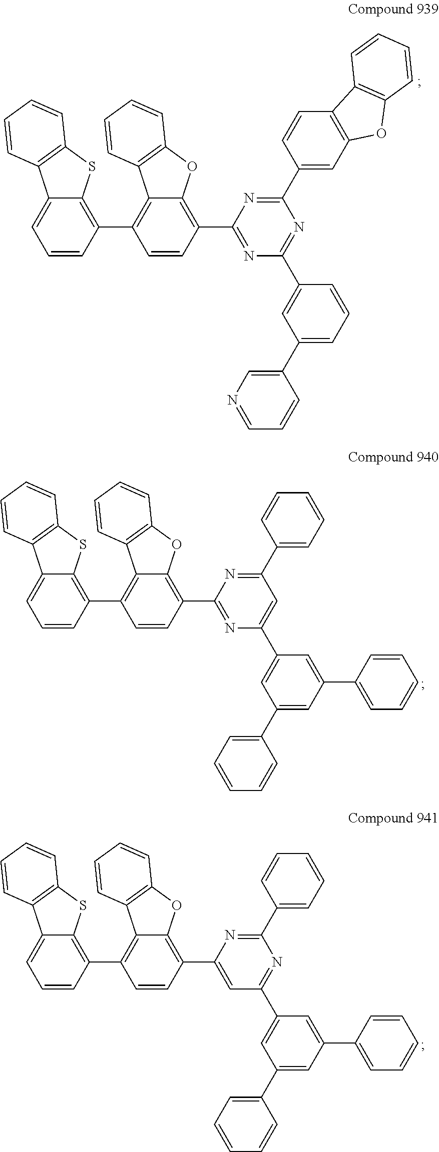 [SMILES: c1cncc(-c2cccc(-c3nc(-c4ccc5c(c4)oc4ccccc45)nc(-c4ccc(-c5cccc6c5sc5ccccc56)c5c4oc4ccccc45)n3)c2)c1 ; c1ccc(-c2cc(-c3ccccc3)cc(-c3cc(-c4ccccc4)nc(-c4ccc(-c5cccc6c5sc5ccccc56)c5c4oc4ccccc45)n3)c2)cc1 ; c1ccc(-c2cc(-c3ccccc3)cc(-c3cc(-c4ccc(-c5cccc6c5sc5ccccc56)c5c4oc4ccccc45)nc(-c4ccccc4)n3)c2)cc1]

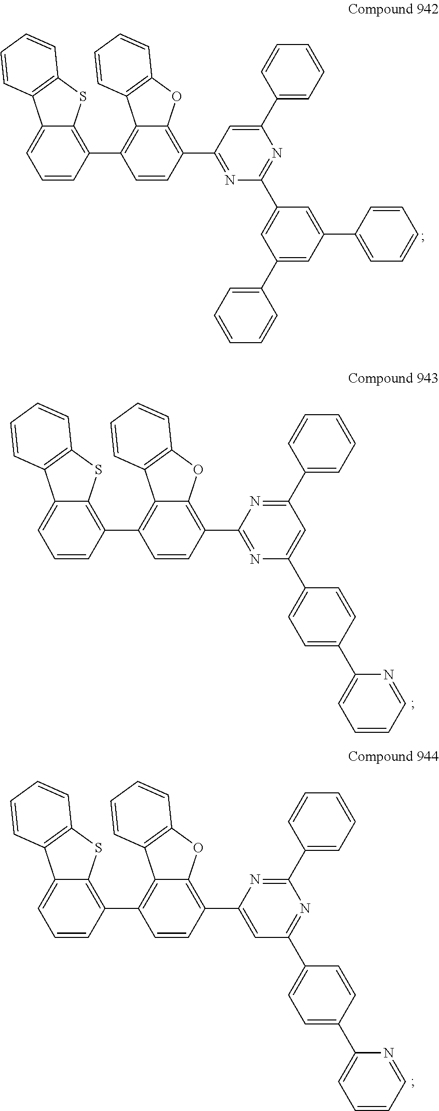 [SMILES: c1ccc(-c2cc(-c3ccccc3)cc(-c3nc(-c4ccccc4)cc(-c4ccc(-c5cccc6c5sc5ccccc56)c5c4oc4ccccc45)n3)c2)cc1 ; c1ccc(-c2cc(-c3ccc(-c4ccccn4)cc3)nc(-c3ccc(-c4cccc5c4sc4ccccc45)c4c3oc3ccccc34)n2)cc1 ; c1ccc(-c2nc(-c3ccc(-c4ccccn4)cc3)cc(-c3ccc(-c4cccc5c4sc4ccccc45)c4c3oc3ccccc34)n2)cc1]

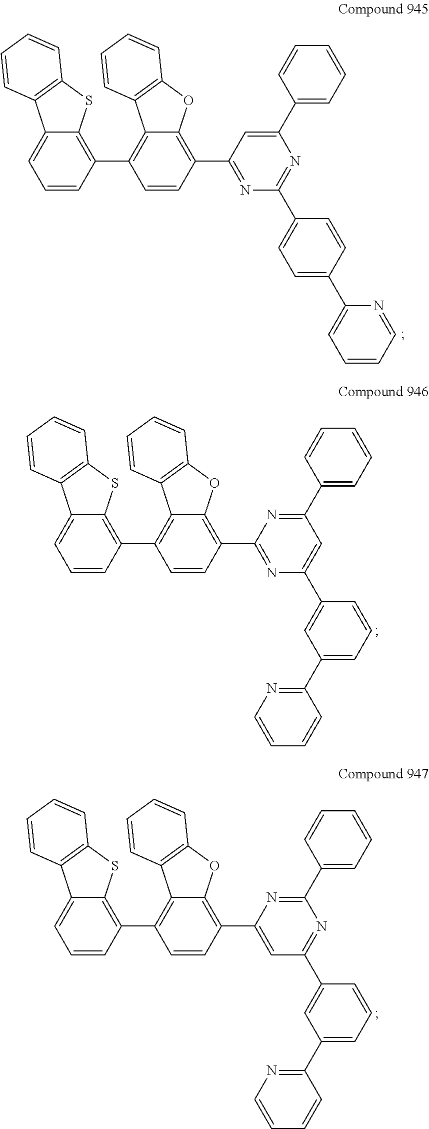 [SMILES: c1ccc(-c2cc(-c3ccc(-c4cccc5c4sc4ccccc45)c4c3oc3ccccc34)nc(-c3ccc(-c4ccccn4)cc3)n2)cc1 ; c1ccc(-c2cc(-c3cccc(-c4ccccn4)c3)nc(-c3ccc(-c4cccc5c4sc4ccccc45)c4c3oc3ccccc34)n2)cc1 ; c1ccc(-c2nc(-c3cccc(-c4ccccn4)c3)cc(-c3ccc(-c4cccc5c4sc4ccccc45)c4c3oc3ccccc34)n2)cc1]

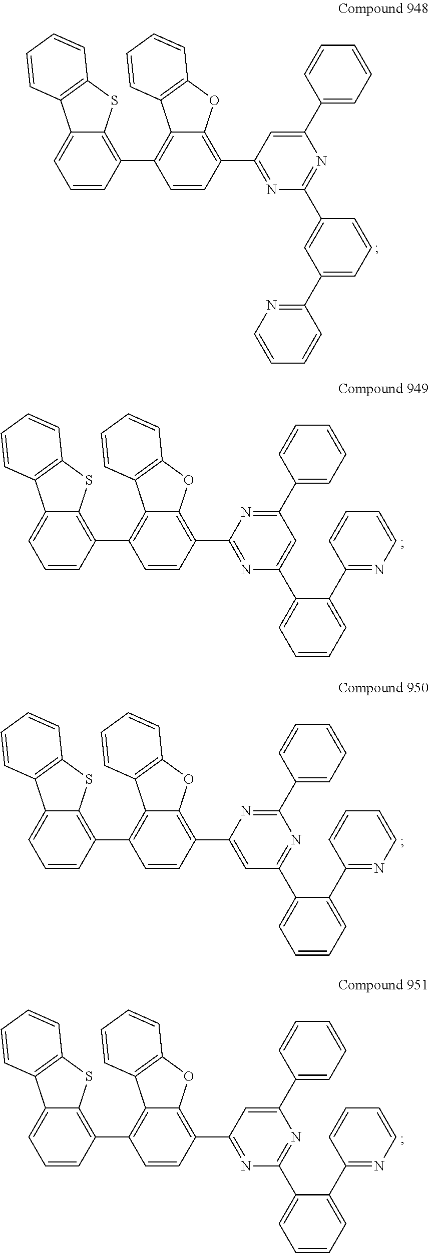 [SMILES: c1ccc(-c2cc(-c3ccc(-c4cccc5c4sc4ccccc45)c4c3oc3ccccc34)nc(-c3cccc(-c4ccccn4)c3)n2)cc1 ; c1ccc(-c2cc(-c3ccccc3-c3ccccn3)nc(-c3ccc(-c4cccc5c4sc4ccccc45)c4c3oc3ccccc34)n2)cc1 ; c1ccc(-c2nc(-c3ccccc3-c3ccccn3)cc(-c3ccc(-c4cccc5c4sc4ccccc45)c4c3oc3ccccc34)n2)cc1 ; c1ccc(-c2cc(-c3ccc(-c4cccc5c4sc4ccccc45)c4c3oc3ccccc34)nc(-c3ccccc3-c3ccccn3)n2)cc1]

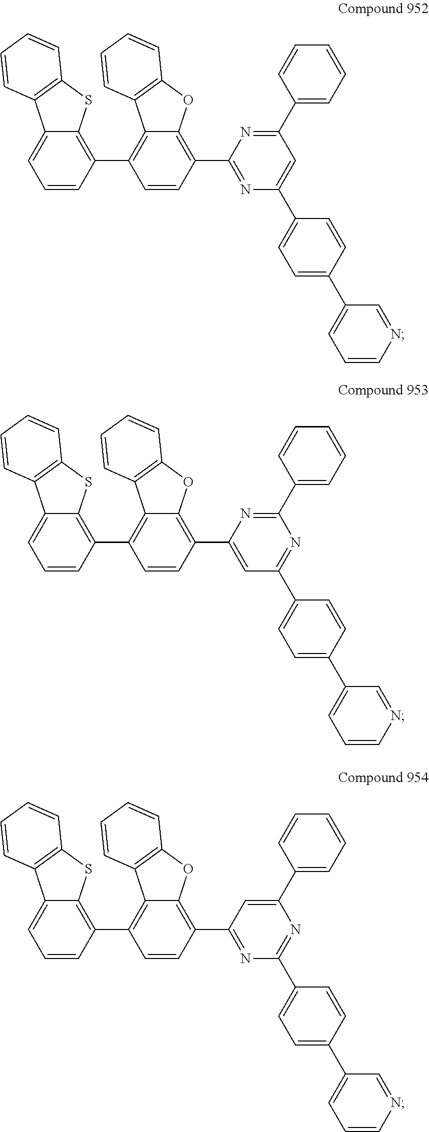 [SMILES: c1ccc(-c2cc(-c3ccc(-c4cccnc4)cc3)nc(-c3ccc(-c4cccc5c4sc4ccccc45)c4c3oc3ccccc34)n2)cc1 ; c1ccc(-c2nc(-c3ccc(-c4cccnc4)cc3)cc(-c3ccc(-c4cccc5c4sc4ccccc45)c4c3oc3ccccc34)n2)cc1 ; c1ccc(-c2cc(-c3ccc(-c4cccc5c4sc4ccccc45)c4c3oc3ccccc34)nc(-c3ccc(-c4cccnc4)cc3)n2)cc1]

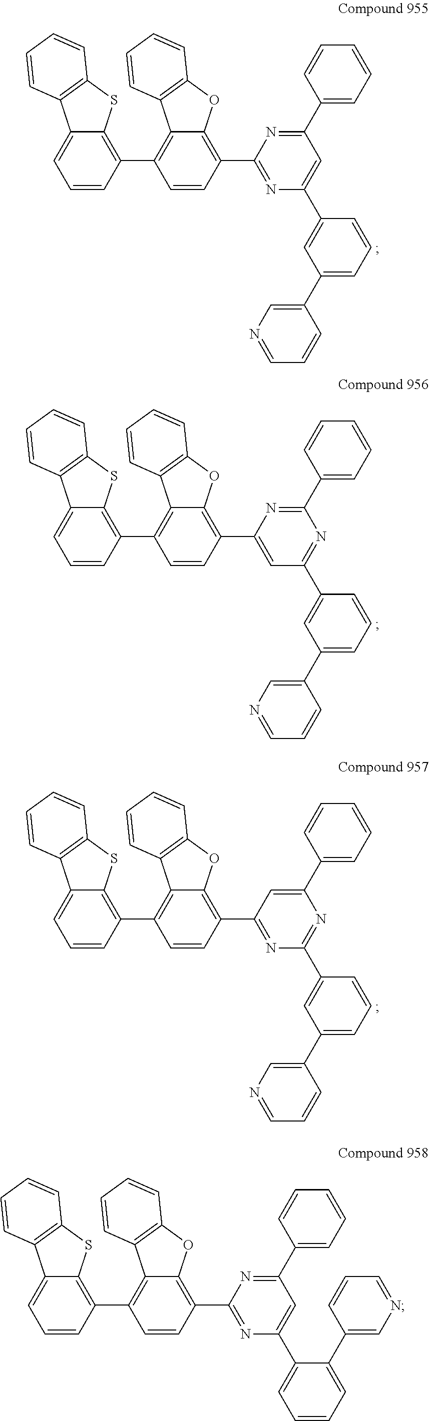 [SMILES: c1ccc(-c2cc(-c3cccc(-c4cccnc4)c3)nc(-c3ccc(-c4cccc5c4sc4ccccc45)c4c3oc3ccccc34)n2)cc1 ; c1ccc(-c2nc(-c3cccc(-c4cccnc4)c3)cc(-c3ccc(-c4cccc5c4sc4ccccc45)c4c3oc3ccccc34)n2)cc1 ; c1ccc(-c2cc(-c3ccc(-c4cccc5c4sc4ccccc45)c4c3oc3ccccc34)nc(-c3cccc(-c4cccnc4)c3)n2)cc1 ; c1ccc(-c2cc(-c3ccccc3-c3cccnc3)nc(-c3ccc(-c4cccc5c4sc4ccccc45)c4c3oc3ccccc34)n2)cc1]

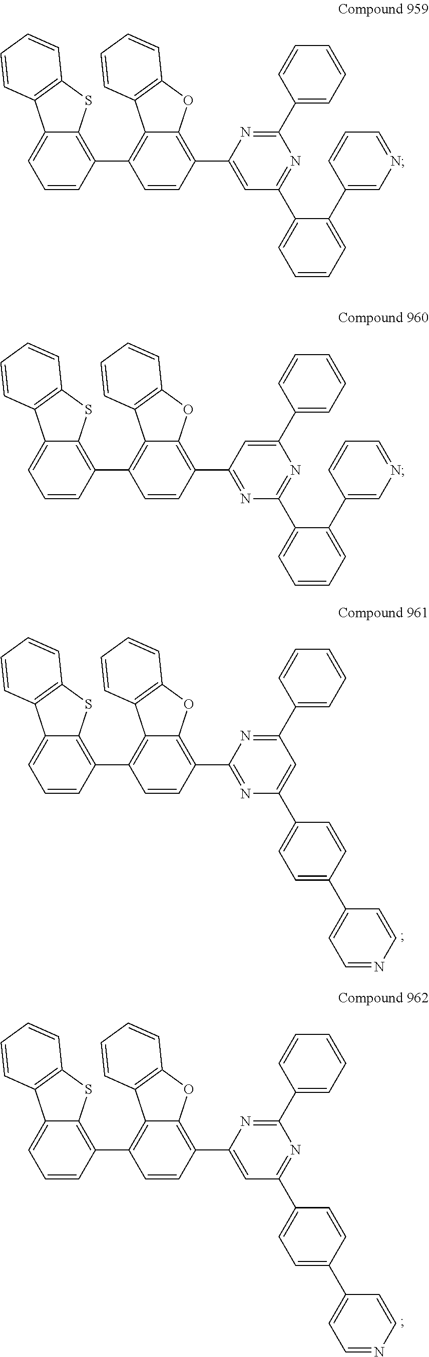 [SMILES: c1ccc(-c2nc(-c3ccccc3-c3cccnc3)cc(-c3ccc(-c4cccc5c4sc4ccccc45)c4c3oc3ccccc34)n2)cc1 ; c1ccc(-c2cc(-c3ccc(-c4cccc5c4sc4ccccc45)c4c3oc3ccccc34)nc(-c3ccccc3-c3cccnc3)n2)cc1 ; c1ccc(-c2cc(-c3ccc(-c4ccncc4)cc3)nc(-c3ccc(-c4cccc5c4sc4ccccc45)c4c3oc3ccccc34)n2)cc1 ; c1ccc(-c2nc(-c3ccc(-c4ccncc4)cc3)cc(-c3ccc(-c4cccc5c4sc4ccccc45)c4c3oc3ccccc34)n2)cc1]

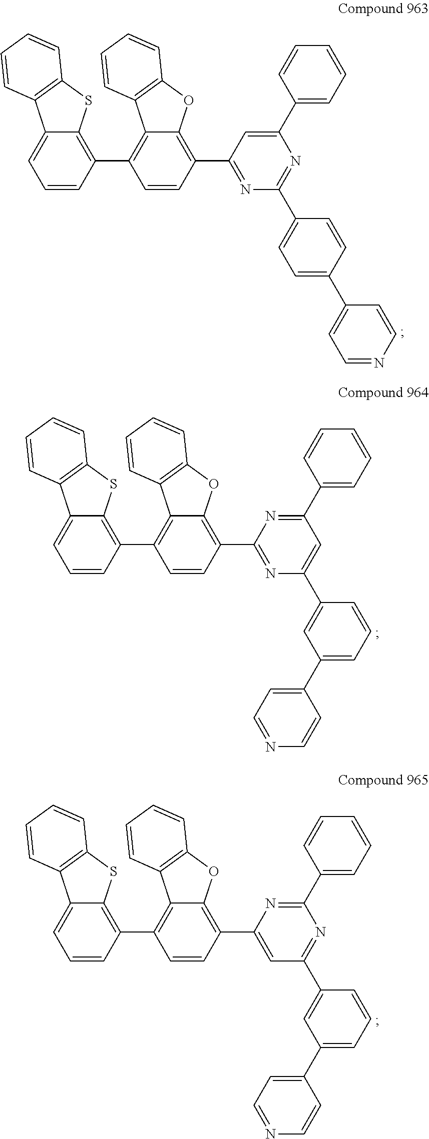 [SMILES: c1ccc(-c2cc(-c3ccc(-c4cccc5c4sc4ccccc45)c4c3oc3ccccc34)nc(-c3ccc(-c4ccncc4)cc3)n2)cc1 ; c1ccc(-c2cc(-c3cccc(-c4ccncc4)c3)nc(-c3ccc(-c4cccc5c4sc4ccccc45)c4c3oc3ccccc34)n2)cc1 ; c1ccc(-c2nc(-c3cccc(-c4ccncc4)c3)cc(-c3ccc(-c4cccc5c4sc4ccccc45)c4c3oc3ccccc34)n2)cc1]

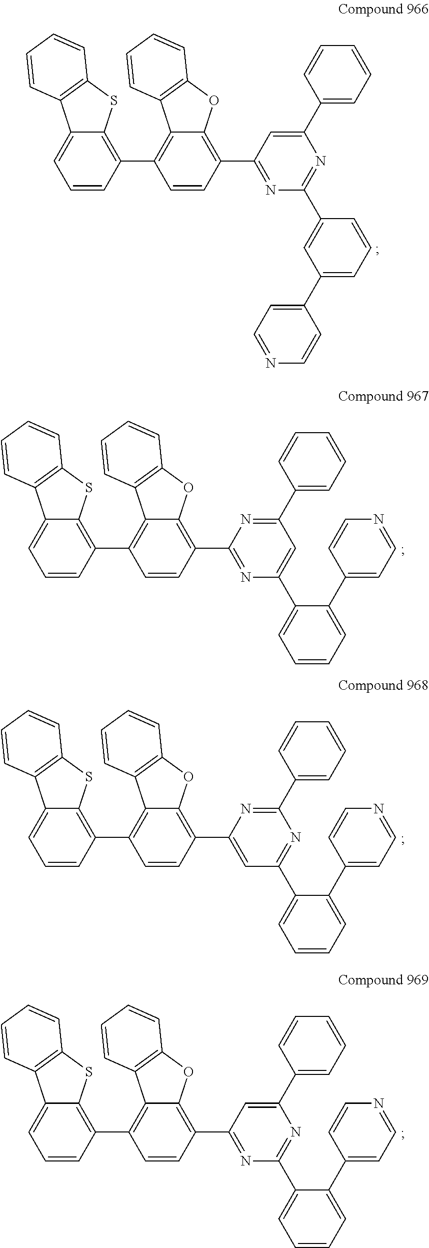 [SMILES: c1ccc(-c2cc(-c3ccc(-c4cccc5c4sc4ccccc45)c4c3oc3ccccc34)nc(-c3cccc(-c4ccncc4)c3)n2)cc1 ; c1ccc(-c2cc(-c3ccccc3-c3ccncc3)nc(-c3ccc(-c4cccc5c4sc4ccccc45)c4c3oc3ccccc34)n2)cc1 ; c1ccc(-c2nc(-c3ccccc3-c3ccncc3)cc(-c3ccc(-c4cccc5c4sc4ccccc45)c4c3oc3ccccc34)n2)cc1 ; c1ccc(-c2cc(-c3ccc(-c4cccc5c4sc4ccccc45)c4c3oc3ccccc34)nc(-c3ccccc3-c3ccncc3)n2)cc1]

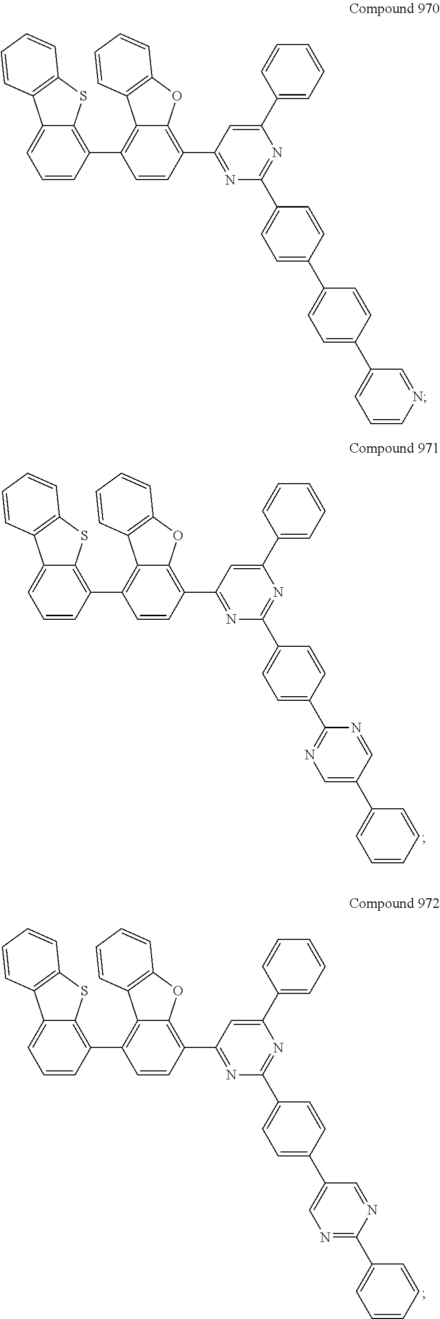 [SMILES: c1ccc(-c2cc(-c3ccc(-c4cccc5c4sc4ccccc45)c4c3oc3ccccc34)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6cccc7c6sc6ccccc67)c6c5oc5ccccc56)n4)cc3)nc2)cc1 ; c1ccc(-c2cc(-c3ccc(-c4cccc5c4sc4ccccc45)c4c3oc3ccccc34)nc(-c3ccc(-c4cnc(-c5ccccc5)nc4)cc3)n2)cc1]

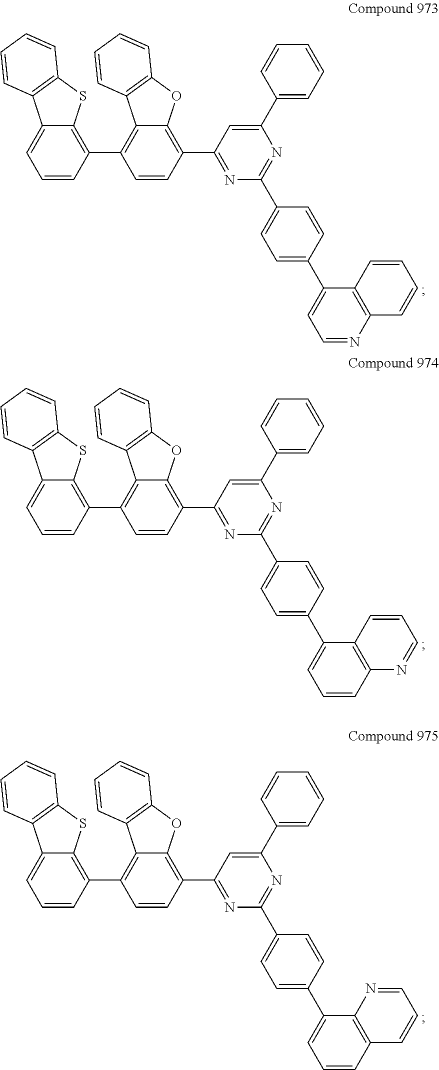 [SMILES: c1ccc(-c2cc(-c3ccc(-c4cccc5c4sc4ccccc45)c4c3oc3ccccc34)nc(-c3ccc(-c4ccnc5ccccc45)cc3)n2)cc1 ; c1ccc(-c2cc(-c3ccc(-c4cccc5c4sc4ccccc45)c4c3oc3ccccc34)nc(-c3ccc(-c4cccc5ncccc45)cc3)n2)cc1 ; c1ccc(-c2cc(-c3ccc(-c4cccc5c4sc4ccccc45)c4c3oc3ccccc34)nc(-c3ccc(-c4cccc5cccnc45)cc3)n2)cc1]

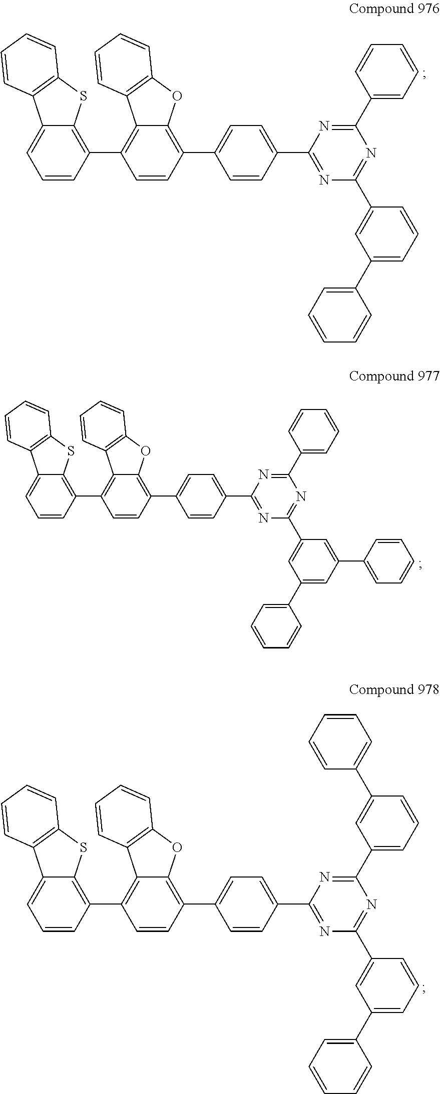 [SMILES: c1ccc(-c2cccc(-c3nc(-c4ccccc4)nc(-c4ccc(-c5ccc(-c6cccc7c6sc6ccccc67)c6c5oc5ccccc56)cc4)n3)c2)cc1 ; c1ccc(-c2cc(-c3ccccc3)cc(-c3nc(-c4ccccc4)nc(-c4ccc(-c5ccc(-c6cccc7c6sc6ccccc67)c6c5oc5ccccc56)cc4)n3)c2)cc1 ; c1ccc(-c2cccc(-c3nc(-c4ccc(-c5ccc(-c6cccc7c6sc6ccccc67)c6c5oc5ccccc56)cc4)nc(-c4cccc(-c5ccccc5)c4)n3)c2)cc1]

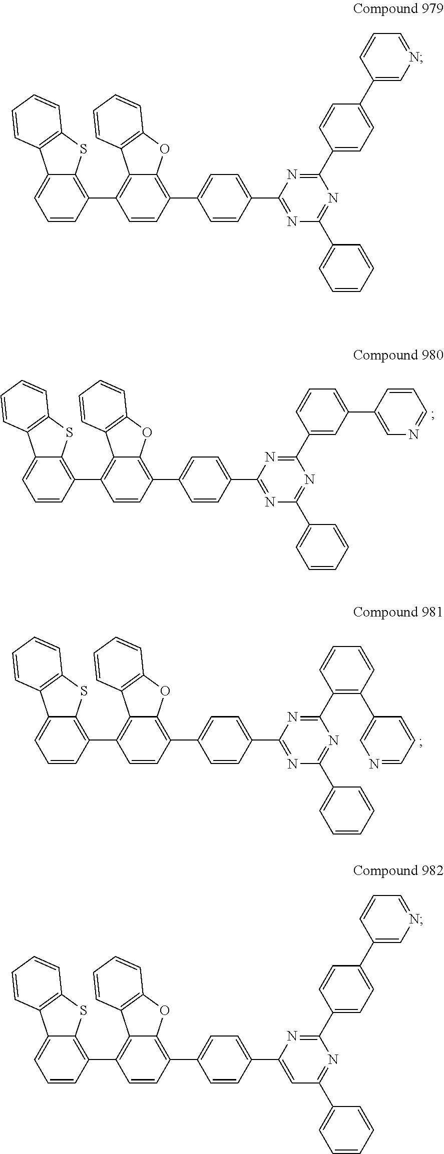 [SMILES: c1ccc(-c2nc(-c3ccc(-c4cccnc4)cc3)nc(-c3ccc(-c4ccc(-c5cccc6c5sc5ccccc56)c5c4oc4ccccc45)cc3)n2)cc1 ; c1ccc(-c2nc(-c3ccc(-c4ccc(-c5cccc6c5sc5ccccc56)c5c4oc4ccccc45)cc3)nc(-c3cccc(-c4cccnc4)c3)n2)cc1 ; c1ccc(-c2nc(-c3ccc(-c4ccc(-c5cccc6c5sc5ccccc56)c5c4oc4ccccc45)cc3)nc(-c3ccccc3-c3cccnc3)n2)cc1 ; c1ccc(-c2cc(-c3ccc(-c4ccc(-c5cccc6c5sc5ccccc56)c5c4oc4ccccc45)cc3)nc(-c3ccc(-c4cccnc4)cc3)n2)cc1]

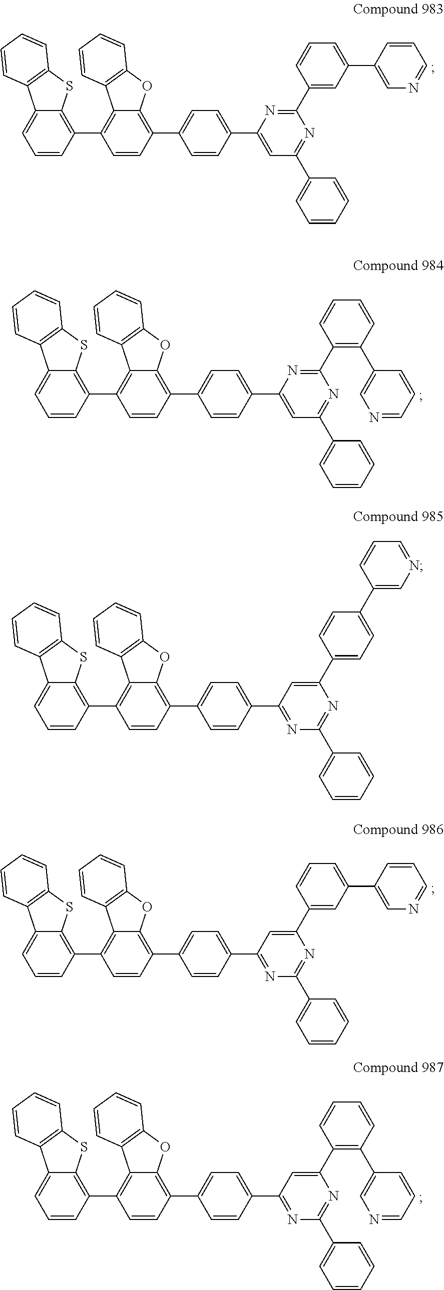 [SMILES: c1ccc(-c2cc(-c3ccc(-c4ccc(-c5cccc6c5sc5ccccc56)c5c4oc4ccccc45)cc3)nc(-c3cccc(-c4cccnc4)c3)n2)cc1 ; c1ccc(-c2cc(-c3ccc(-c4ccc(-c5cccc6c5sc5ccccc56)c5c4oc4ccccc45)cc3)nc(-c3ccccc3-c3cccnc3)n2)cc1 ; c1ccc(-c2nc(-c3ccc(-c4cccnc4)cc3)cc(-c3ccc(-c4ccc(-c5cccc6c5sc5ccccc56)c5c4oc4ccccc45)cc3)n2)cc1 ; c1ccc(-c2nc(-c3ccc(-c4ccc(-c5cccc6c5sc5ccccc56)c5c4oc4ccccc45)cc3)cc(-c3cccc(-c4cccnc4)c3)n2)cc1 ; c1ccc(-c2nc(-c3ccc(-c4ccc(-c5cccc6c5sc5ccccc56)c5c4oc4ccccc45)cc3)cc(-c3ccccc3-c3cccnc3)n2)cc1]

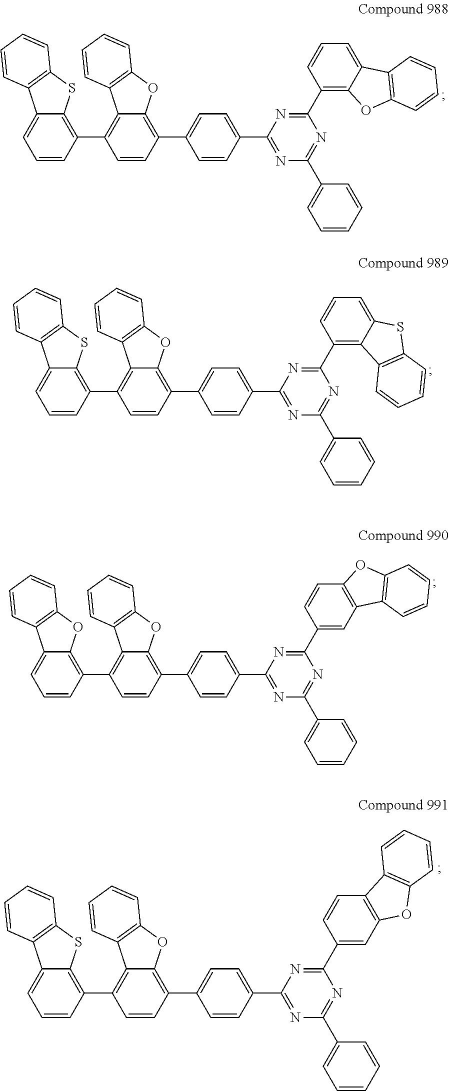 [SMILES: c1ccc(-c2nc(-c3ccc(-c4ccc(-c5cccc6c5sc5ccccc56)c5c4oc4ccccc45)cc3)nc(-c3cccc4c3oc3ccccc34)n2)cc1 ; c1ccc(-c2nc(-c3ccc(-c4ccc(-c5cccc6c5sc5ccccc56)c5c4oc4ccccc45)cc3)nc(-c3cccc4sc5ccccc5c34)n2)cc1 ; c1ccc(-c2nc(-c3ccc(-c4ccc(-c5cccc6c5oc5ccccc56)c5c4oc4ccccc45)cc3)nc(-c3ccc4oc5ccccc5c4c3)n2)cc1 ; c1ccc(-c2nc(-c3ccc(-c4ccc(-c5cccc6c5sc5ccccc56)c5c4oc4ccccc45)cc3)nc(-c3ccc4c(c3)oc3ccccc34)n2)cc1]

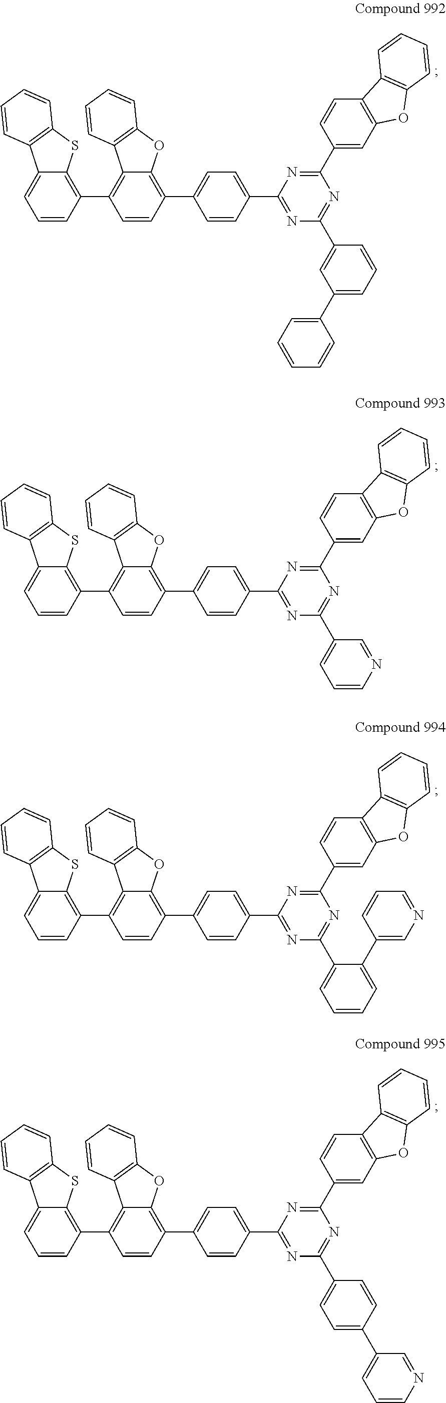 [SMILES: c1ccc(-c2cccc(-c3nc(-c4ccc(-c5ccc(-c6cccc7c6sc6ccccc67)c6c5oc5ccccc56)cc4)nc(-c4ccc5c(c4)oc4ccccc45)n3)c2)cc1 ; c1cncc(-c2nc(-c3ccc(-c4ccc(-c5cccc6c5sc5ccccc56)c5c4oc4ccccc45)cc3)nc(-c3ccc4c(c3)oc3ccccc34)n2)c1 ; c1cncc(-c2ccccc2-c2nc(-c3ccc(-c4ccc(-c5cccc6c5sc5ccccc56)c5c4oc4ccccc45)cc3)nc(-c3ccc4c(c3)oc3ccccc34)n2)c1 ; c1cncc(-c2ccc(-c3nc(-c4ccc(-c5ccc(-c6cccc7c6sc6ccccc67)c6c5oc5ccccc56)cc4)nc(-c4ccc5c(c4)oc4ccccc45)n3)cc2)c1]

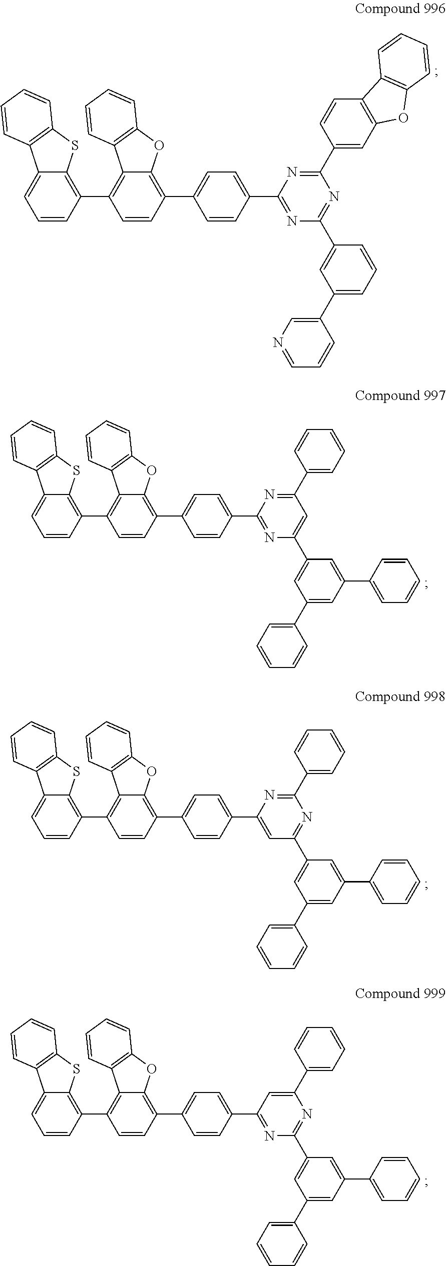 [SMILES: c1cncc(-c2cccc(-c3nc(-c4ccc(-c5ccc(-c6cccc7c6sc6ccccc67)c6c5oc5ccccc56)cc4)nc(-c4ccc5c(c4)oc4ccccc45)n3)c2)c1 ; c1ccc(-c2cc(-c3ccccc3)cc(-c3cc(-c4ccccc4)nc(-c4ccc(-c5ccc(-c6cccc7c6sc6ccccc67)c6c5oc5ccccc56)cc4)n3)c2)cc1 ; c1ccc(-c2cc(-c3ccccc3)cc(-c3cc(-c4ccc(-c5ccc(-c6cccc7c6sc6ccccc67)c6c5oc5ccccc56)cc4)nc(-c4ccccc4)n3)c2)cc1 ; c1ccc(-c2cc(-c3ccccc3)cc(-c3nc(-c4ccccc4)cc(-c4ccc(-c5ccc(-c6cccc7c6sc6ccccc67)c6c5oc5ccccc56)cc4)n3)c2)cc1]

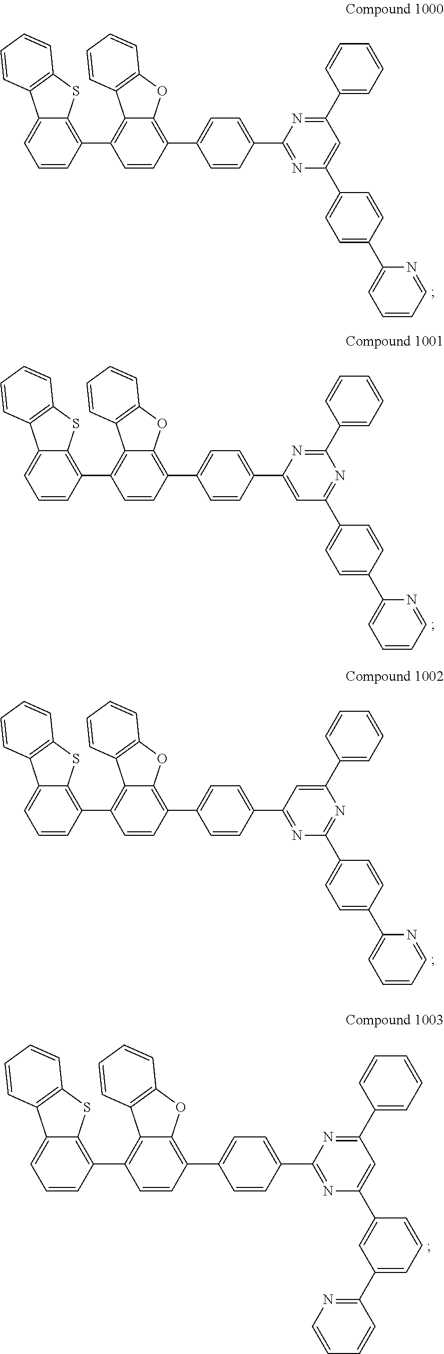 [SMILES: c1ccc(-c2cc(-c3ccc(-c4ccccn4)cc3)nc(-c3ccc(-c4ccc(-c5cccc6c5sc5ccccc56)c5c4oc4ccccc45)cc3)n2)cc1 ; c1ccc(-c2nc(-c3ccc(-c4ccccn4)cc3)cc(-c3ccc(-c4ccc(-c5cccc6c5sc5ccccc56)c5c4oc4ccccc45)cc3)n2)cc1 ; c1ccc(-c2cc(-c3ccc(-c4ccc(-c5cccc6c5sc5ccccc56)c5c4oc4ccccc45)cc3)nc(-c3ccc(-c4ccccn4)cc3)n2)cc1 ; c1ccc(-c2cc(-c3cccc(-c4ccccn4)c3)nc(-c3ccc(-c4ccc(-c5cccc6c5sc5ccccc56)c5c4oc4ccccc45)cc3)n2)cc1]

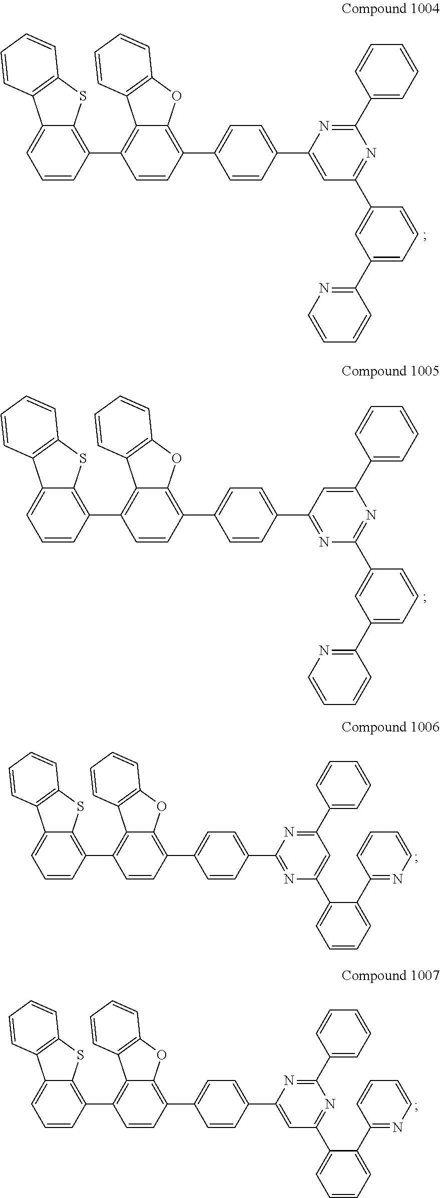 [SMILES: c1ccc(-c2nc(-c3ccc(-c4ccc(-c5cccc6c5sc5ccccc56)c5c4oc4ccccc45)cc3)cc(-c3cccc(-c4ccccn4)c3)n2)cc1 ; c1ccc(-c2cc(-c3ccc(-c4ccc(-c5cccc6c5sc5ccccc56)c5c4oc4ccccc45)cc3)nc(-c3cccc(-c4ccccn4)c3)n2)cc1 ; c1ccc(-c2cc(-c3ccccc3-c3ccccn3)nc(-c3ccc(-c4ccc(-c5cccc6c5sc5ccccc56)c5c4oc4ccccc45)cc3)n2)cc1 ; c1ccc(-c2nc(-c3ccc(-c4ccc(-c5cccc6c5sc5ccccc56)c5c4oc4ccccc45)cc3)cc(-c3ccccc3-c3ccccn3)n2)cc1]

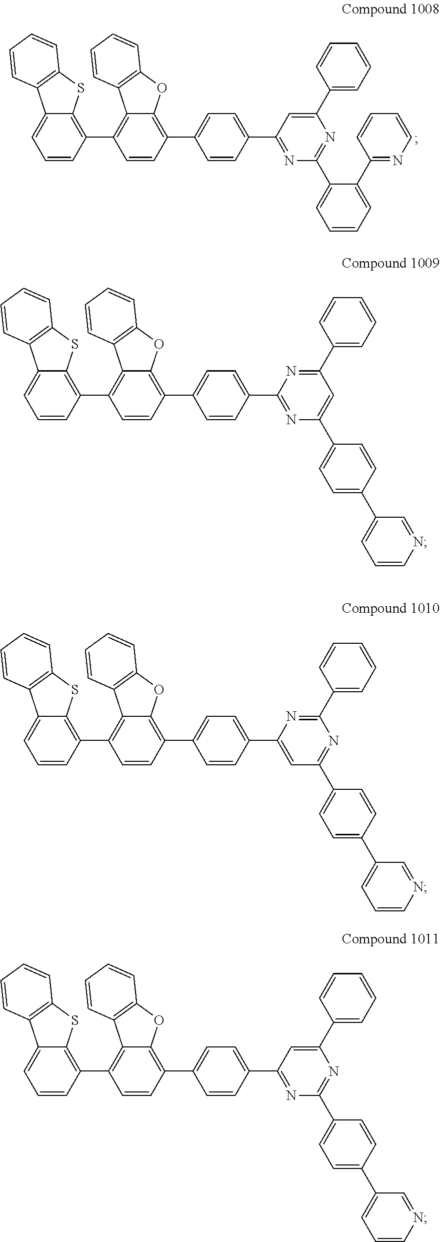 [SMILES: c1ccc(-c2cc(-c3ccc(-c4ccc(-c5cccc6c5sc5ccccc56)c5c4oc4ccccc45)cc3)nc(-c3ccccc3-c3ccccn3)n2)cc1 ; c1ccc(-c2cc(-c3ccc(-c4cccnc4)cc3)nc(-c3ccc(-c4ccc(-c5cccc6c5sc5ccccc56)c5c4oc4ccccc45)cc3)n2)cc1 ; c1ccc(-c2nc(-c3ccc(-c4cccnc4)cc3)cc(-c3ccc(-c4ccc(-c5cccc6c5sc5ccccc56)c5c4oc4ccccc45)cc3)n2)cc1 ; c1ccc(-c2cc(-c3ccc(-c4ccc(-c5cccc6c5sc5ccccc56)c5c4oc4ccccc45)cc3)nc(-c3ccc(-c4cccnc4)cc3)n2)cc1]

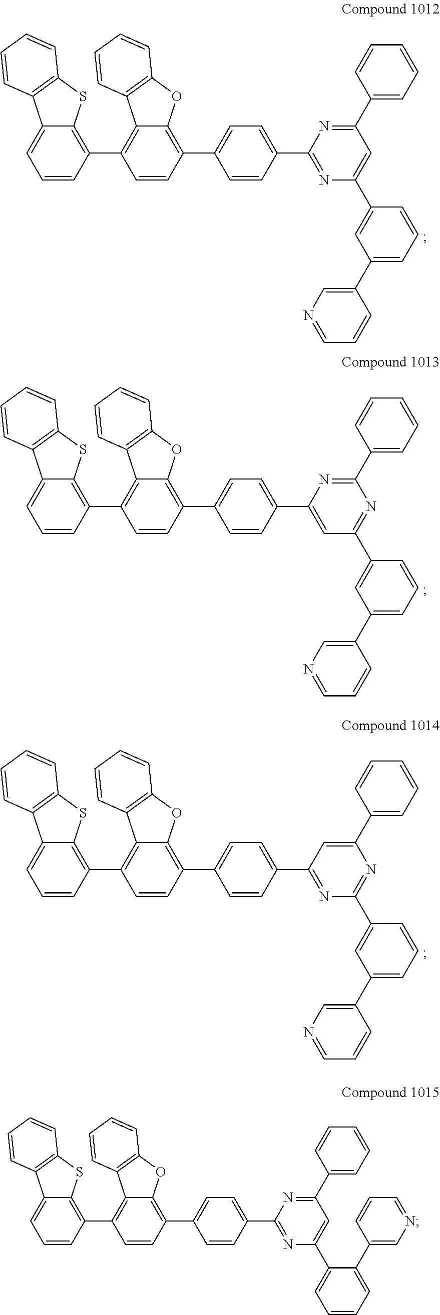 [SMILES: c1ccc(-c2cc(-c3cccc(-c4cccnc4)c3)nc(-c3ccc(-c4ccc(-c5cccc6c5sc5ccccc56)c5c4oc4ccccc45)cc3)n2)cc1 ; c1ccc(-c2nc(-c3ccc(-c4ccc(-c5cccc6c5sc5ccccc56)c5c4oc4ccccc45)cc3)cc(-c3cccc(-c4cccnc4)c3)n2)cc1 ; c1ccc(-c2cc(-c3ccc(-c4ccc(-c5cccc6c5sc5ccccc56)c5c4oc4ccccc45)cc3)nc(-c3cccc(-c4cccnc4)c3)n2)cc1 ; c1ccc(-c2cc(-c3ccccc3-c3cccnc3)nc(-c3ccc(-c4ccc(-c5cccc6c5sc5ccccc56)c5c4oc4ccccc45)cc3)n2)cc1]

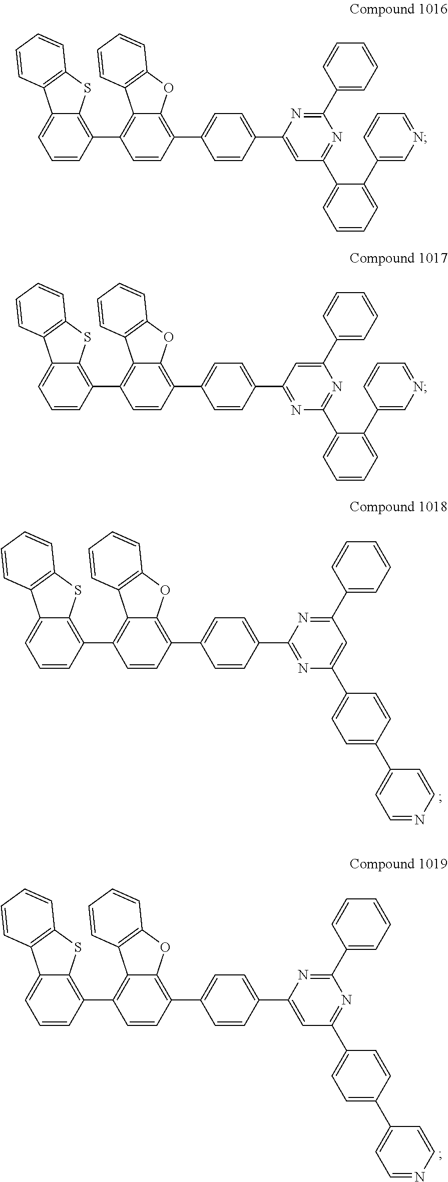 [SMILES: c1ccc(-c2nc(-c3ccc(-c4ccc(-c5cccc6c5sc5ccccc56)c5c4oc4ccccc45)cc3)cc(-c3ccccc3-c3cccnc3)n2)cc1 ; c1ccc(-c2cc(-c3ccc(-c4ccc(-c5cccc6c5sc5ccccc56)c5c4oc4ccccc45)cc3)nc(-c3ccccc3-c3cccnc3)n2)cc1 ; c1ccc(-c2cc(-c3ccc(-c4ccncc4)cc3)nc(-c3ccc(-c4ccc(-c5cccc6c5sc5ccccc56)c5c4oc4ccccc45)cc3)n2)cc1 ; c1ccc(-c2nc(-c3ccc(-c4ccncc4)cc3)cc(-c3ccc(-c4ccc(-c5cccc6c5sc5ccccc56)c5c4oc4ccccc45)cc3)n2)cc1]

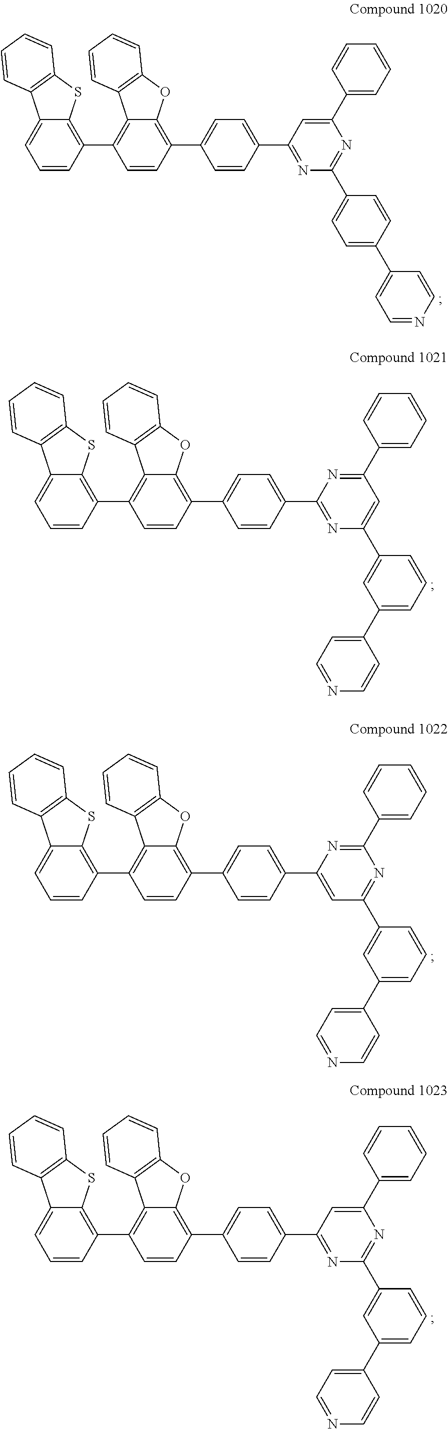 [SMILES: c1ccc(-c2cc(-c3ccc(-c4ccc(-c5cccc6c5sc5ccccc56)c5c4oc4ccccc45)cc3)nc(-c3ccc(-c4ccncc4)cc3)n2)cc1 ; c1ccc(-c2cc(-c3cccc(-c4ccncc4)c3)nc(-c3ccc(-c4ccc(-c5cccc6c5sc5ccccc56)c5c4oc4ccccc45)cc3)n2)cc1 ; c1ccc(-c2nc(-c3ccc(-c4ccc(-c5cccc6c5sc5ccccc56)c5c4oc4ccccc45)cc3)cc(-c3cccc(-c4ccncc4)c3)n2)cc1 ; c1ccc(-c2cc(-c3ccc(-c4ccc(-c5cccc6c5sc5ccccc56)c5c4oc4ccccc45)cc3)nc(-c3cccc(-c4ccncc4)c3)n2)cc1]

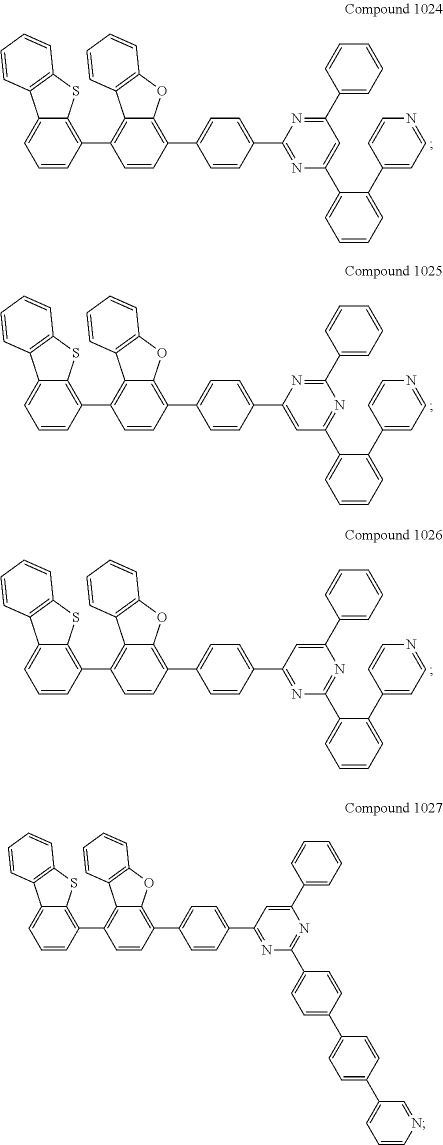 [SMILES: c1ccc(-c2cc(-c3ccccc3-c3ccncc3)nc(-c3ccc(-c4ccc(-c5cccc6c5sc5ccccc56)c5c4oc4ccccc45)cc3)n2)cc1 ; c1ccc(-c2nc(-c3ccc(-c4ccc(-c5cccc6c5sc5ccccc56)c5c4oc4ccccc45)cc3)cc(-c3ccccc3-c3ccncc3)n2)cc1 ; c1ccc(-c2cc(-c3ccc(-c4ccc(-c5cccc6c5sc5ccccc56)c5c4oc4ccccc45)cc3)nc(-c3ccccc3-c3ccncc3)n2)cc1 ; c1ccc(-c2cc(-c3ccc(-c4ccc(-c5cccc6c5sc5ccccc56)c5c4oc4ccccc45)cc3)nc(-c3ccc(-c4ccc(-c5cccnc5)cc4)cc3)n2)cc1]

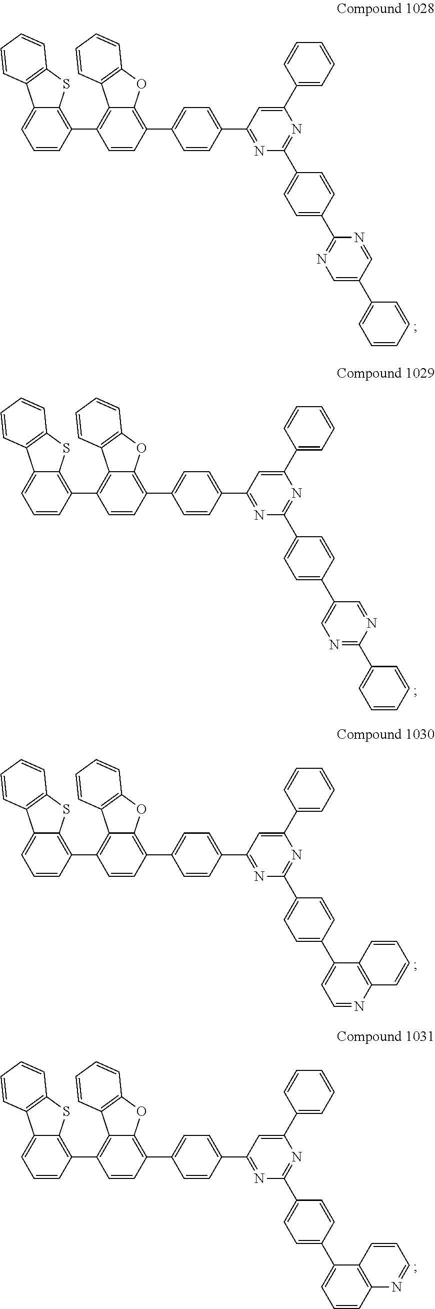 [SMILES: c1ccc(-c2cnc(-c3ccc(-c4nc(-c5ccccc5)cc(-c5ccc(-c6ccc(-c7cccc8c7sc7ccccc78)c7c6oc6ccccc67)cc5)n4)cc3)nc2)cc1 ; c1ccc(-c2cc(-c3ccc(-c4ccc(-c5cccc6c5sc5ccccc56)c5c4oc4ccccc45)cc3)nc(-c3ccc(-c4cnc(-c5ccccc5)nc4)cc3)n2)cc1 ; c1ccc(-c2cc(-c3ccc(-c4ccc(-c5cccc6c5sc5ccccc56)c5c4oc4ccccc45)cc3)nc(-c3ccc(-c4ccnc5ccccc45)cc3)n2)cc1 ; c1ccc(-c2cc(-c3ccc(-c4ccc(-c5cccc6c5sc5ccccc56)c5c4oc4ccccc45)cc3)nc(-c3ccc(-c4cccc5ncccc45)cc3)n2)cc1]

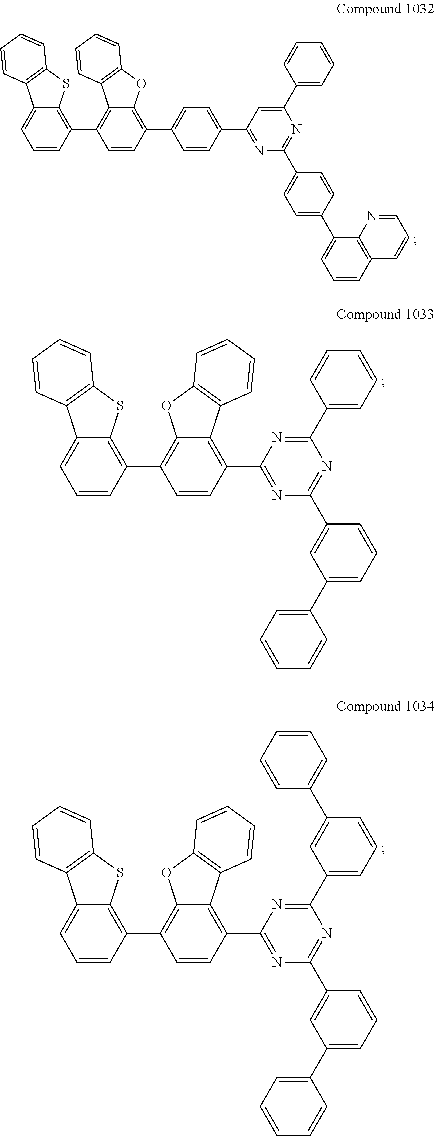 [SMILES: c1ccc(-c2cc(-c3ccc(-c4ccc(-c5cccc6c5sc5ccccc56)c5c4oc4ccccc45)cc3)nc(-c3ccc(-c4cccc5cccnc45)cc3)n2)cc1 ; c1ccc(-c2cccc(-c3nc(-c4ccccc4)nc(-c4ccc(-c5cccc6c5sc5ccccc56)c5oc6ccccc6c45)n3)c2)cc1 ; c1ccc(-c2cccc(-c3nc(-c4cccc(-c5ccccc5)c4)nc(-c4ccc(-c5cccc6c5sc5ccccc56)c5oc6ccccc6c45)n3)c2)cc1]

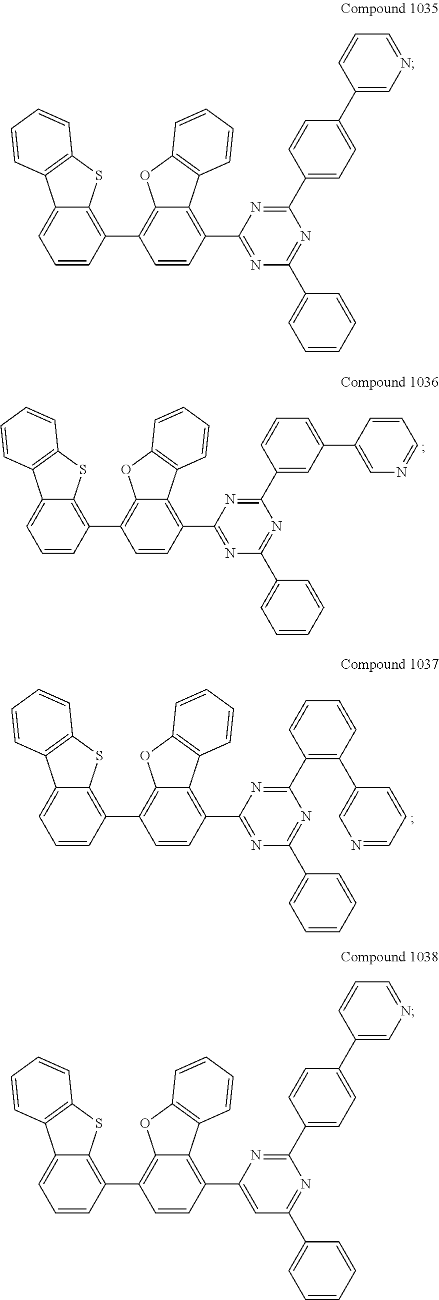 [SMILES: c1ccc(-c2nc(-c3ccc(-c4cccnc4)cc3)nc(-c3ccc(-c4cccc5c4sc4ccccc45)c4oc5ccccc5c34)n2)cc1 ; c1ccc(-c2nc(-c3cccc(-c4cccnc4)c3)nc(-c3ccc(-c4cccc5c4sc4ccccc45)c4oc5ccccc5c34)n2)cc1 ; c1ccc(-c2nc(-c3ccccc3-c3cccnc3)nc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3ccc(-c4cccnc4)cc3)n2)cc1]

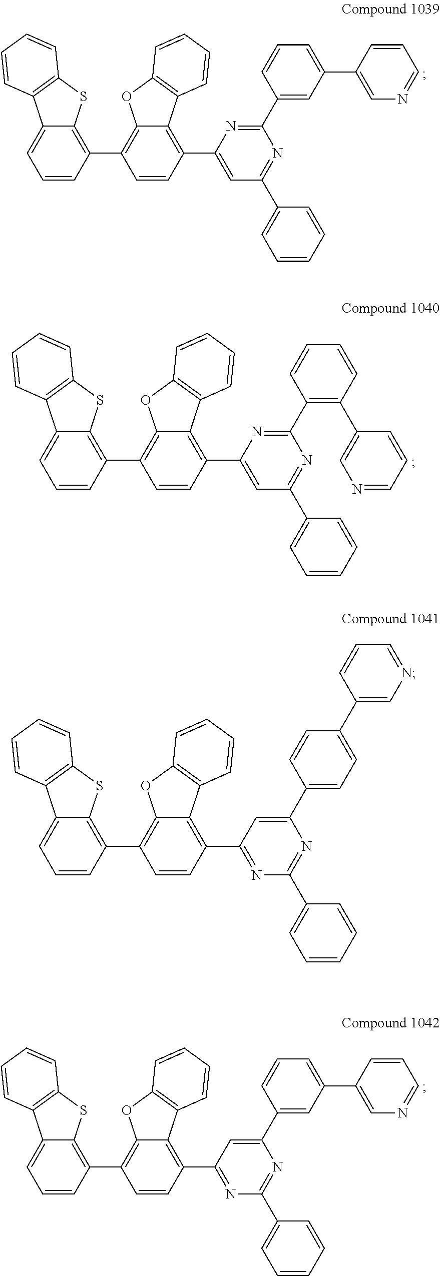 [SMILES: c1ccc(-c2cc(-c3ccc(-c4cccc5c4sc4ccccc45)c4oc5ccccc5c34)nc(-c3cccc(-c4cccnc4)c3)n2)cc1 ; c1ccc(-c2cc(-c3ccc(-c4cccc5c4sc4ccccc45)c4oc5ccccc5c34)nc(-c3ccccc3-c3cccnc3)n2)cc1 ; c1ccc(-c2nc(-c3ccc(-c4cccnc4)cc3)cc(-c3ccc(-c4cccc5c4sc4ccccc45)c4oc5ccccc5c34)n2)cc1 ; c1ccc(-c2nc(-c3cccc(-c4cccnc4)c3)cc(-c3ccc(-c4cccc5c4sc4ccccc45)c4oc5ccccc5c34)n2)cc1]

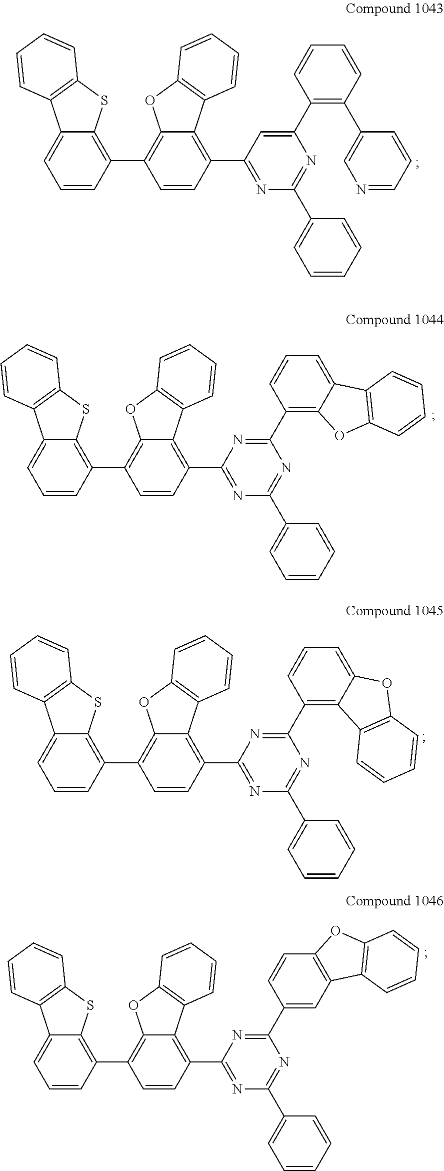 [SMILES: c1ccc(-c2nc(-c3ccccc3-c3cccnc3)cc(-c3ccc(-c4cccc5c4sc4ccccc45)c4oc5ccccc5c34)n2)cc1 ; c1ccc(-c2nc(-c3cccc4c3oc3ccccc34)nc(-c3ccc(-c4cccc5c4sc4ccccc45)c4oc5ccccc5c34)n2)cc1 ; c1ccc(-c2nc(-c3cccc4oc5ccccc5c34)nc(-c3ccc(-c4cccc5c4sc4ccccc45)c4oc5ccccc5c34)n2)cc1 ; c1ccc(-c2nc(-c3ccc4oc5ccccc5c4c3)nc(-c3ccc(-c4cccc5c4sc4ccccc45)c4oc5ccccc5c34)n2)cc1]

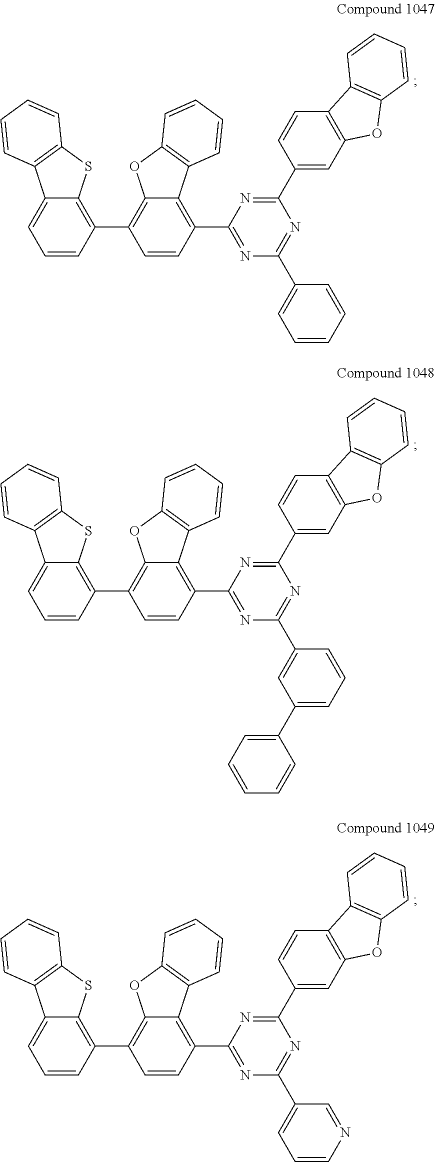 [SMILES: c1ccc(-c2nc(-c3ccc4c(c3)oc3ccccc34)nc(-c3ccc(-c4cccc5c4sc4ccccc45)c4oc5ccccc5c34)n2)cc1 ; c1ccc(-c2cccc(-c3nc(-c4ccc5c(c4)oc4ccccc45)nc(-c4ccc(-c5cccc6c5sc5ccccc56)c5oc6ccccc6c45)n3)c2)cc1 ; c1cncc(-c2nc(-c3ccc4c(c3)oc3ccccc34)nc(-c3ccc(-c4cccc5c4sc4ccccc45)c4oc5ccccc5c34)n2)c1]

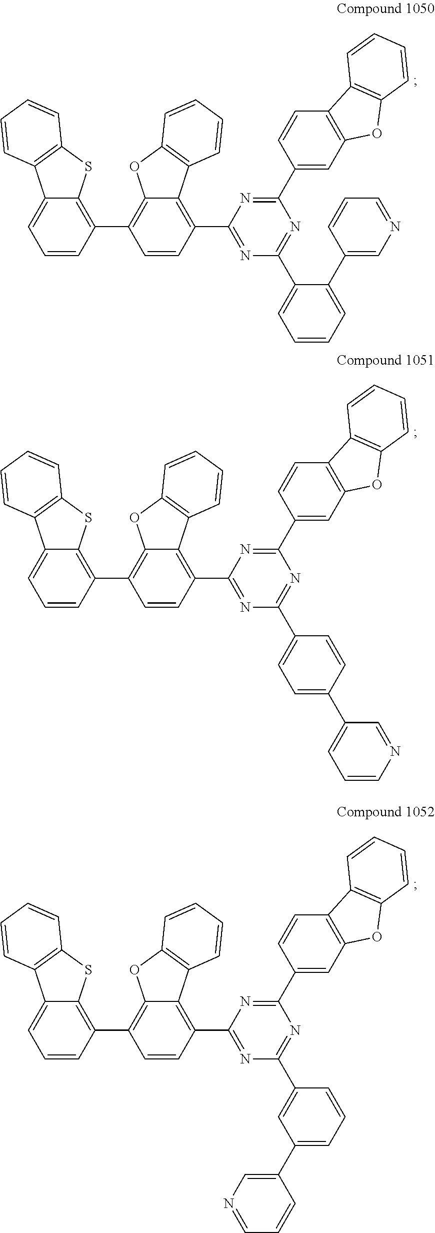 [SMILES: c1cncc(-c2ccccc2-c2nc(-c3ccc4c(c3)oc3ccccc34)nc(-c3ccc(-c4cccc5c4sc4ccccc45)c4oc5ccccc5c34)n2)c1 ; c1cncc(-c2ccc(-c3nc(-c4ccc5c(c4)oc4ccccc45)nc(-c4ccc(-c5cccc6c5sc5ccccc56)c5oc6ccccc6c45)n3)cc2)c1 ; c1cncc(-c2cccc(-c3nc(-c4ccc5c(c4)oc4ccccc45)nc(-c4ccc(-c5cccc6c5sc5ccccc56)c5oc6ccccc6c45)n3)c2)c1]

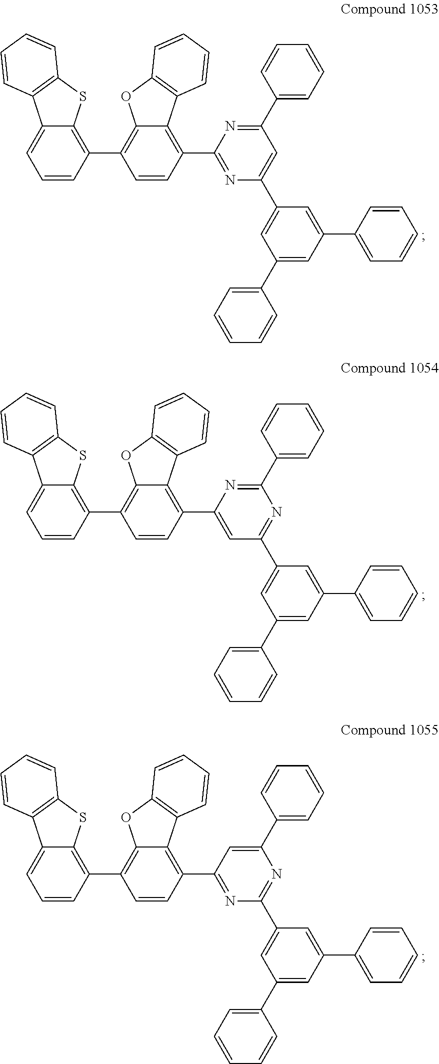 [SMILES: c1ccc(-c2cc(-c3ccccc3)cc(-c3cc(-c4ccccc4)nc(-c4ccc(-c5cccc6c5sc5ccccc56)c5oc6ccccc6c45)n3)c2)cc1 ; c1ccc(-c2cc(-c3ccccc3)cc(-c3cc(-c4ccc(-c5cccc6c5sc5ccccc56)c5oc6ccccc6c45)nc(-c4ccccc4)n3)c2)cc1 ; c1ccc(-c2cc(-c3ccccc3)cc(-c3nc(-c4ccccc4)cc(-c4ccc(-c5cccc6c5sc5ccccc56)c5oc6ccccc6c45)n3)c2)cc1]

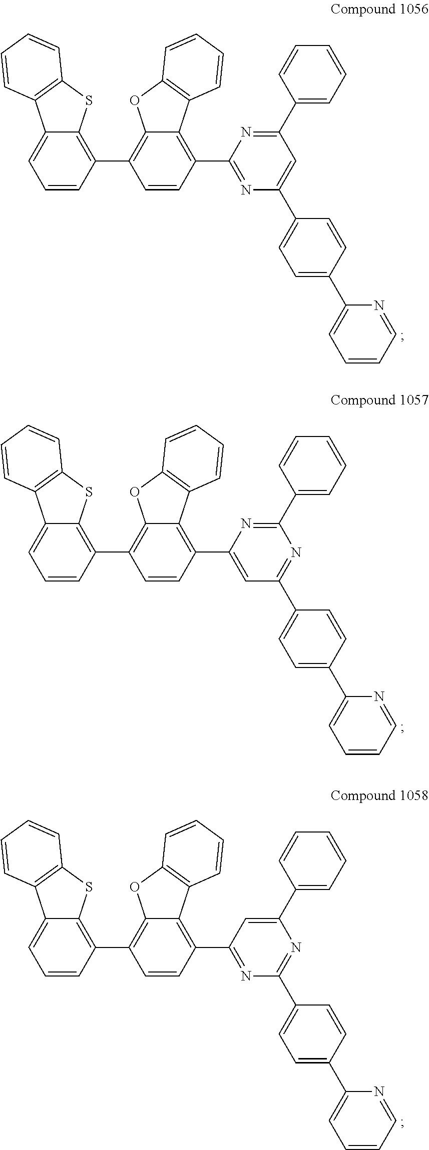 [SMILES: c1ccc(-c2cc(-c3ccc(-c4ccccn4)cc3)nc(-c3ccc(-c4cccc5c4sc4ccccc45)c4oc5ccccc5c34)n2)cc1 ; c1ccc(-c2nc(-c3ccc(-c4ccccn4)cc3)cc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3ccc(-c4ccccn4)cc3)n2)cc1]

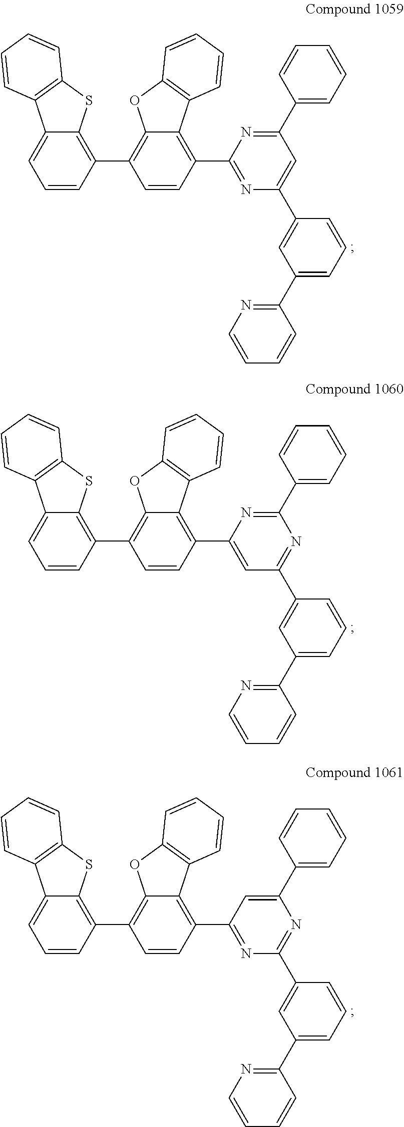 [SMILES: c1ccc(-c2cc(-c3cccc(-c4ccccn4)c3)nc(-c3ccc(-c4cccc5c4sc4ccccc45)c4oc5ccccc5c34)n2)cc1 ; c1ccc(-c2nc(-c3cccc(-c4ccccn4)c3)cc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3cccc(-c4ccccn4)c3)n2)cc1]

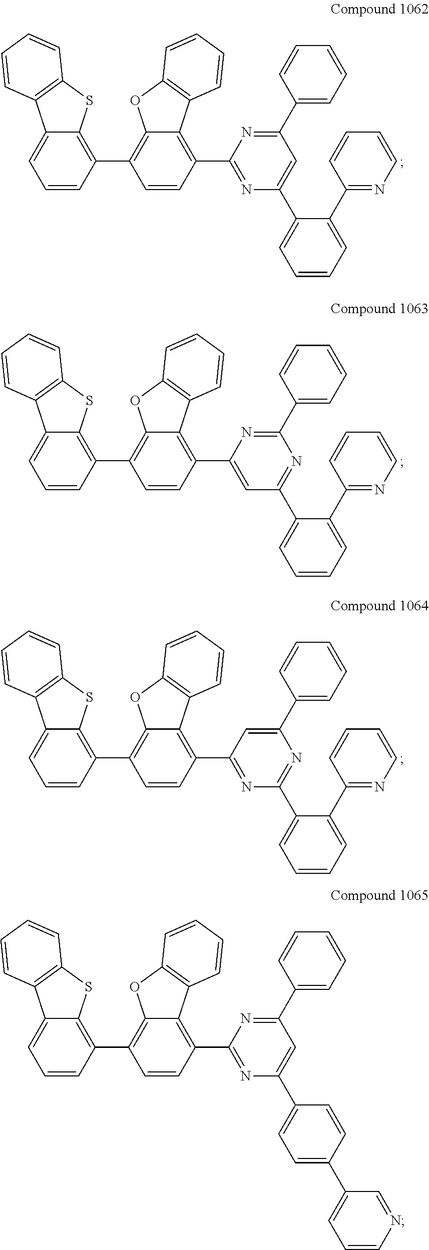 [SMILES: c1ccc(-c2cc(-c3ccccc3-c3ccccn3)nc(-c3ccc(-c4cccc5c4sc4ccccc45)c4oc5ccccc5c34)n2)cc1 ; c1ccc(-c2nc(-c3ccccc3-c3ccccn3)cc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3ccccc3-c3ccccn3)n2)cc1 ; c1ccc(-c2cc(-c3ccc(-c4cccnc4)cc3)nc(-c3ccc(-c4cccc5c4sc4ccccc45)c4oc5ccccc5c34)n2)cc1]

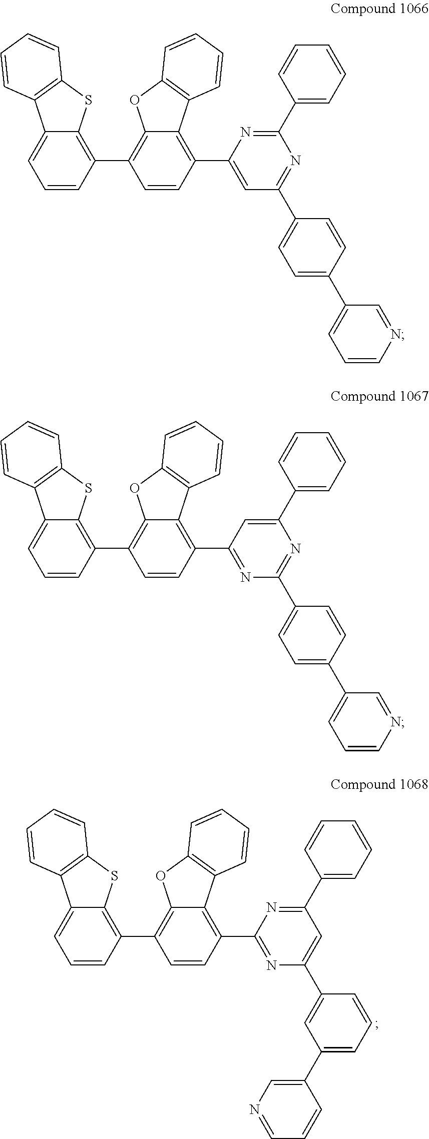 [SMILES: c1ccc(-c2nc(-c3ccc(-c4cccnc4)cc3)cc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3ccc(-c4cccnc4)cc3)n2)cc1 ; c1ccc(-c2cc(-c3cccc(-c4cccnc4)c3)nc(-c3ccc(-c4cccc5c4sc4ccccc45)c4oc5ccccc5c34)n2)cc1]

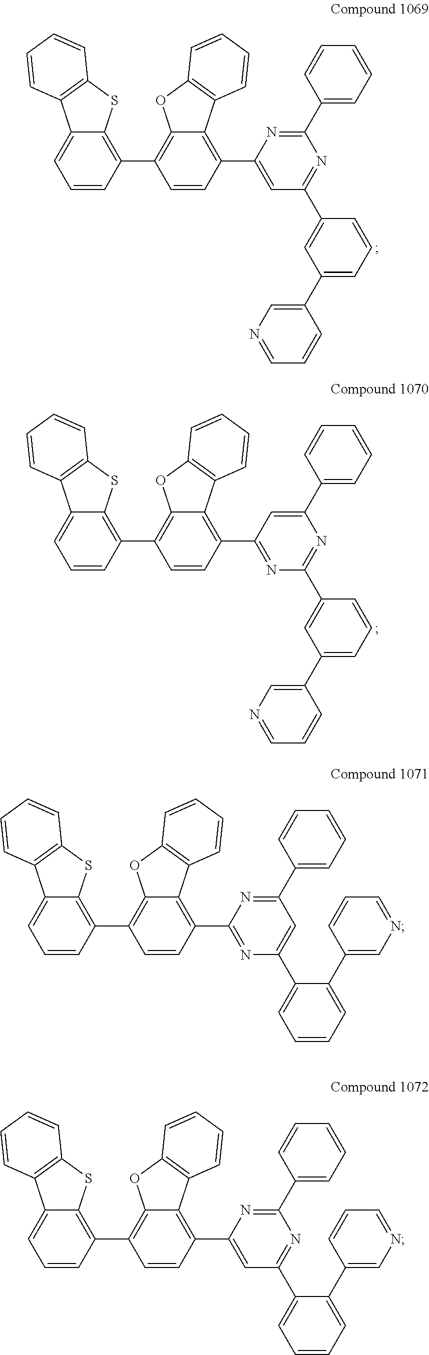 [SMILES: c1ccc(-c2nc(-c3cccc(-c4cccnc4)c3)cc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3cccc(-c4cccnc4)c3)n2)cc1 ; c1ccc(-c2cc(-c3ccccc3-c3cccnc3)nc(-c3ccc(-c4cccc5c4sc4ccccc45)c4oc5ccccc5c34)n2)cc1 ; c1ccc(-c2nc(-c3ccccc3-c3cccnc3)cc(-c3ccc(-c4cccc5c4sc4ccccc45)c4oc5ccccc5c34)n2)cc1]

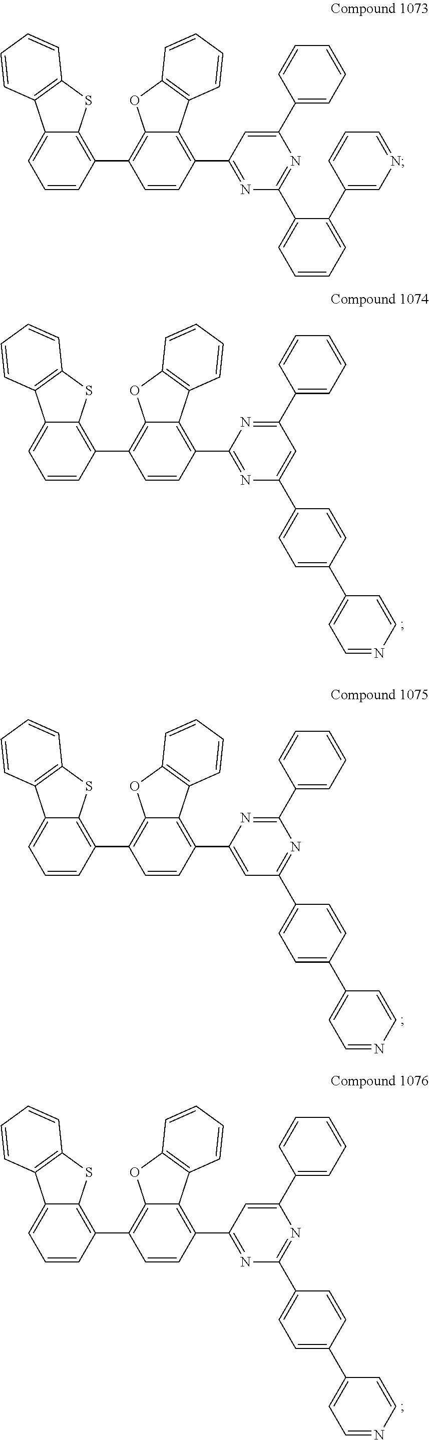 [SMILES: c1ccc(-c2cc(-c3ccc(-c4cccc5c4sc4ccccc45)c4oc5ccccc5c34)nc(-c3ccccc3-c3cccnc3)n2)cc1 ; c1ccc(-c2cc(-c3ccc(-c4ccncc4)cc3)nc(-c3ccc(-c4cccc5c4sc4ccccc45)c4oc5ccccc5c34)n2)cc1 ; c1ccc(-c2nc(-c3ccc(-c4ccncc4)cc3)cc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3ccc(-c4ccncc4)cc3)n2)cc1]

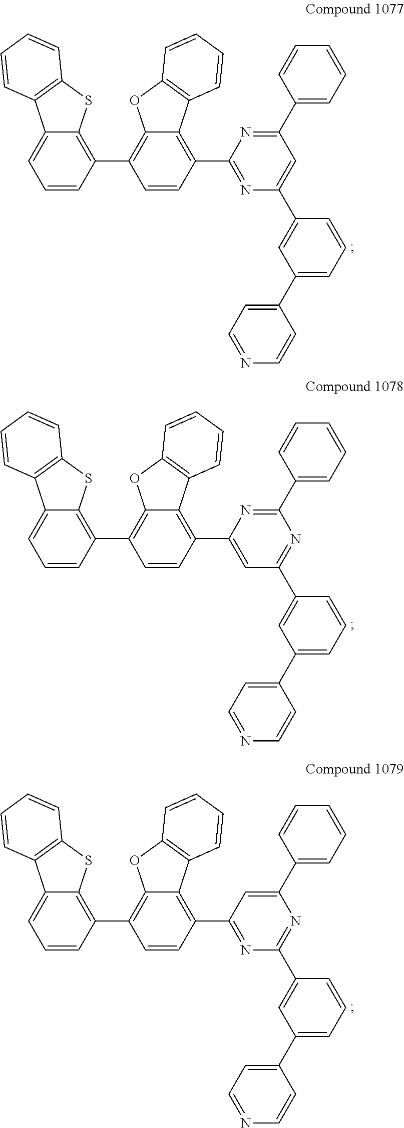 [SMILES: c1ccc(-c2cc(-c3cccc(-c4ccncc4)c3)nc(-c3ccc(-c4cccc5c4sc4ccccc45)c4oc5ccccc5c34)n2)cc1 ; c1ccc(-c2nc(-c3cccc(-c4ccncc4)c3)cc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3cccc(-c4ccncc4)c3)n2)cc1]

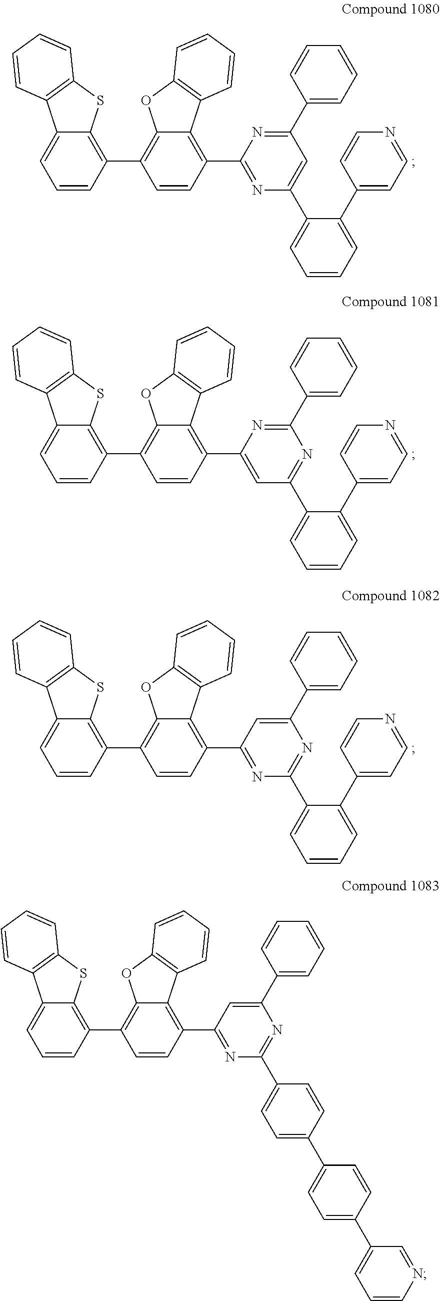 [SMILES: c1ccc(-c2cc(-c3ccccc3-c3ccncc3)nc(-c3ccc(-c4cccc5c4sc4ccccc45)c4oc5ccccc5c34)n2)cc1 ; c1ccc(-c2nc(-c3ccccc3-c3ccncc3)cc(-c3ccc(-c4cccc5c4sc4ccccc45)c4oc5ccccc5c34)n2)cc1 ; c1ccc(-c2cc(-c3ccc(-c4cccc5c4sc4ccccc45)c4oc5ccccc5c34)nc(-c3ccccc3-c3ccncc3)n2)cc1 ; c1ccc(-c2cc(-c3ccc(-c4cccc5c4sc4ccccc45)c4oc5ccccc5c34)nc(-c3ccc(-c4ccc(-c5cccnc5)cc4)cc3)n2)cc1]

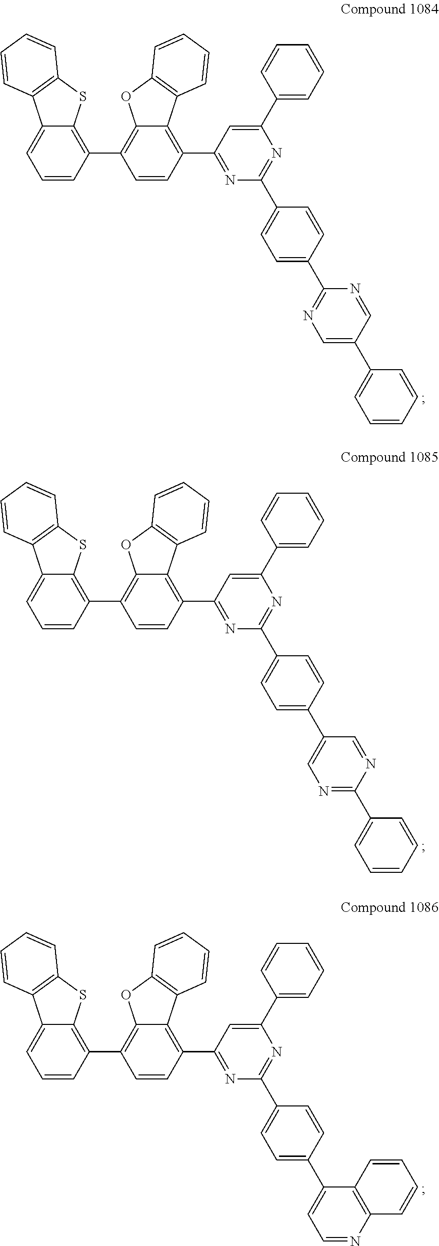 [SMILES: c1ccc(-c2cnc(-c3ccc(-c4nc(-c5ccccc5)cc(-c5ccc(-c6cccc7c6sc6ccccc67)c6oc7ccccc7c56)n4)cc3)nc2)cc1 ; c1ccc(-c2cc(-c3ccc(-c4cccc5c4sc4ccccc45)c4oc5ccccc5c34)nc(-c3ccc(-c4cnc(-c5ccccc5)nc4)cc3)n2)cc1 ; c1ccc(-c2cc(-c3ccc(-c4cccc5c4sc4ccccc45)c4oc5ccccc5c34)nc(-c3ccc(-c4ccnc5ccccc45)cc3)n2)cc1]

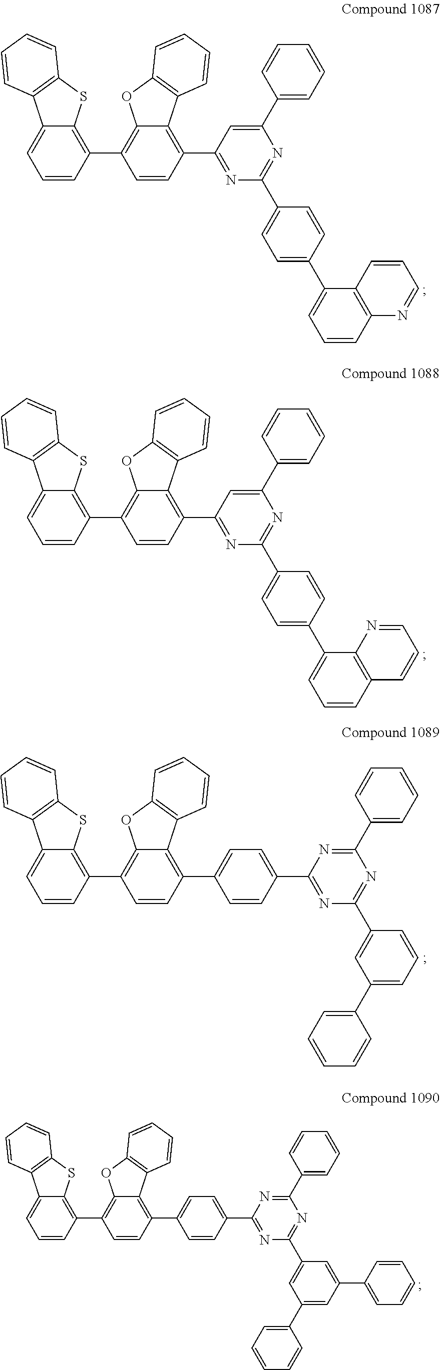 [SMILES: c1ccc(-c2cc(-c3ccc(-c4cccc5c4sc4ccccc45)c4oc5ccccc5c34)nc(-c3ccc(-c4cccc5ncccc45)cc3)n2)cc1 ; c1ccc(-c2cc(-c3ccc(-c4cccc5c4sc4ccccc45)c4oc5ccccc5c34)nc(-c3ccc(-c4cccc5cccnc45)cc3)n2)cc1 ; c1ccc(-c2cccc(-c3nc(-c4ccccc4)nc(-c4ccc(-c5ccc(-c6cccc7c6sc6ccccc67)c6oc7ccccc7c56)cc4)n3)c2)cc1 ; c1ccc(-c2cc(-c3ccccc3)cc(-c3nc(-c4ccccc4)nc(-c4ccc(-c5ccc(-c6cccc7c6sc6ccccc67)c6oc7ccccc7c56)cc4)n3)c2)cc1]

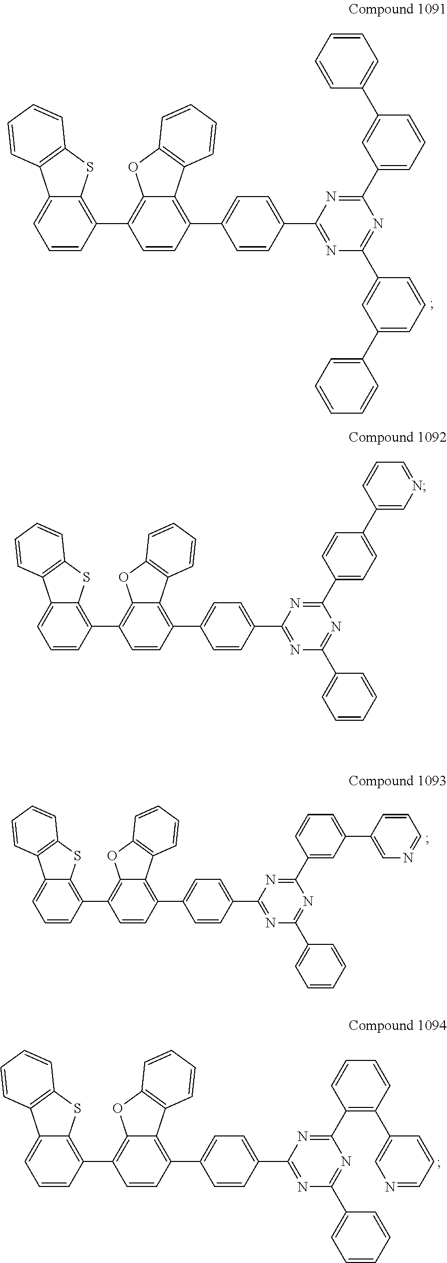 [SMILES: c1ccc(-c2cccc(-c3nc(-c4ccc(-c5ccc(-c6cccc7c6sc6ccccc67)c6oc7ccccc7c56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5cccc6c5sc5ccccc56)c5oc6ccccc6c45)cc3)n2)cc1 ; c1ccc(-c2nc(-c3ccc(-c4ccc(-c5cccc6c5sc5ccccc56)c5oc6ccccc6c45)cc3)nc(-c3cccc(-c4cccnc4)c3)n2)cc1 ; c1ccc(-c2nc(-c3ccc(-c4ccc(-c5cccc6c5sc5ccccc56)c5oc6ccccc6c45)cc3)nc(-c3ccccc3-c3cccnc3)n2)cc1]

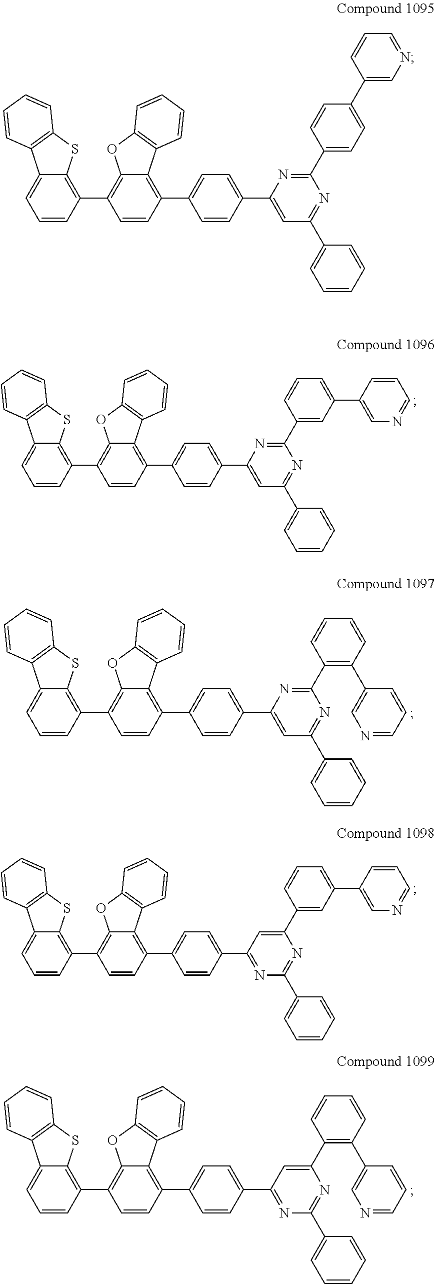 [SMILES: c1ccc(-c2cc(-c3ccc(-c4ccc(-c5cccc6c5sc5ccccc56)c5oc6ccccc6c45)cc3)nc(-c3ccc(-c4cccnc4)cc3)n2)cc1 ; c1ccc(-c2cc(-c3ccc(-c4ccc(-c5cccc6c5sc5ccccc56)c5oc6ccccc6c45)cc3)nc(-c3cccc(-c4cccnc4)c3)n2)cc1 ; c1ccc(-c2cc(-c3ccc(-c4ccc(-c5cccc6c5sc5ccccc56)c5oc6ccccc6c45)cc3)nc(-c3ccccc3-c3cccnc3)n2)cc1 ; c1ccc(-c2nc(-c3ccc(-c4ccc(-c5cccc6c5sc5ccccc56)c5oc6ccccc6c45)cc3)cc(-c3cccc(-c4cccnc4)c3)n2)cc1 ; c1ccc(-c2nc(-c3ccc(-c4ccc(-c5cccc6c5sc5ccccc56)c5oc6ccccc6c45)cc3)cc(-c3ccccc3-c3cccnc3)n2)cc1]

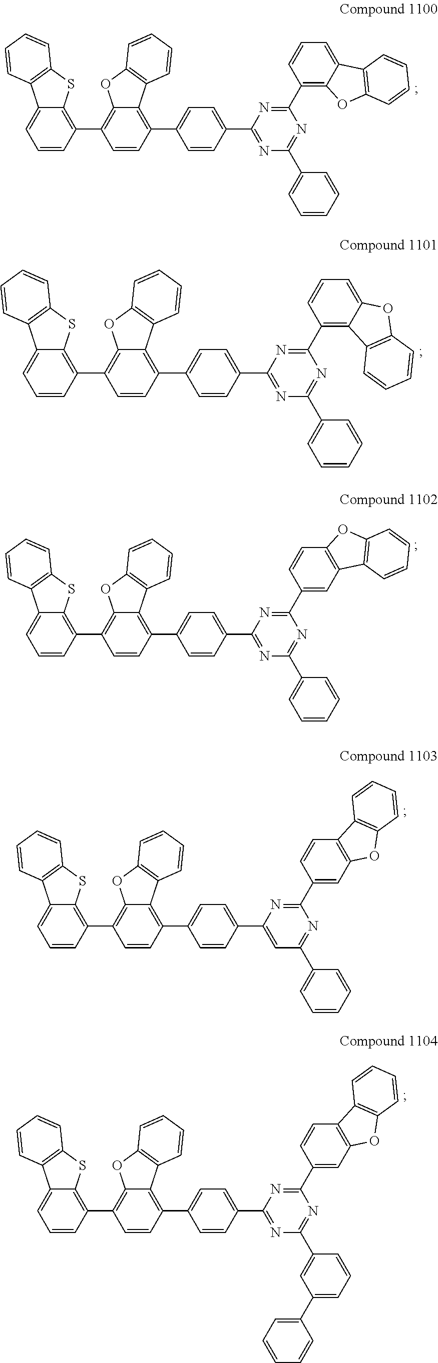

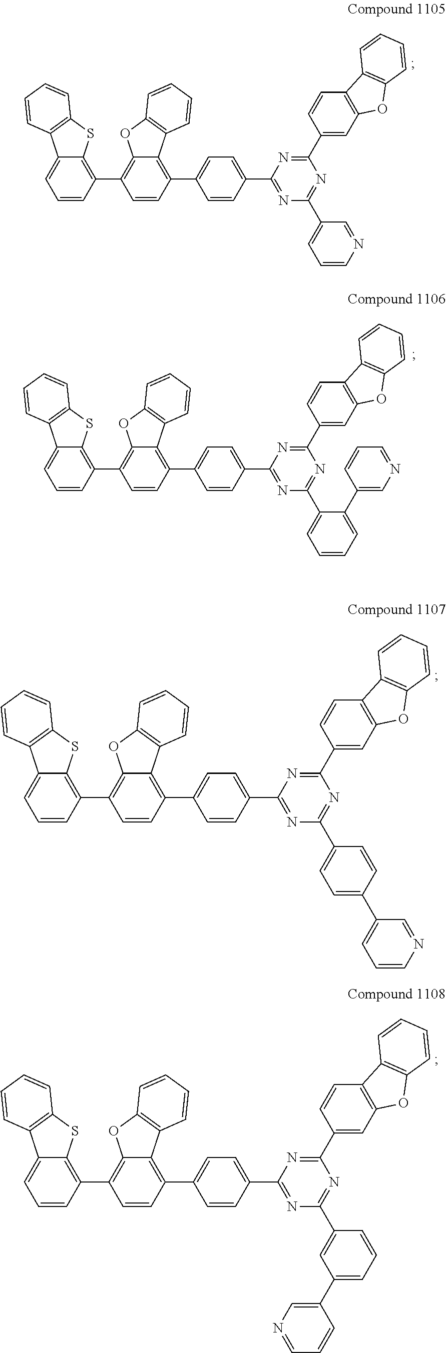 [SMILES: c1cncc(-c2nc(-c3ccc(-c4ccc(-c5cccc6c5sc5ccccc56)c5oc6ccccc6c45)cc3)nc(-c3ccc4c(c3)oc3ccccc34)n2)c1 ; c1cncc(-c2ccccc2-c2nc(-c3ccc(-c4ccc(-c5cccc6c5sc5ccccc56)c5oc6ccccc6c45)cc3)nc(-c3ccc4c(c3)oc3ccccc34)n2)c1 ; c1cncc(-c2ccc(-c3nc(-c4ccc(-c5ccc(-c6cccc7c6sc6ccccc67)c6oc7ccccc7c56)cc4)nc(-c4ccc5c(c4)oc4ccccc45)n3)cc2)c1 ; c1cncc(-c2cccc(-c3nc(-c4ccc(-c5ccc(-c6cccc7c6sc6ccccc67)c6oc7ccccc7c56)cc4)nc(-c4ccc5c(c4)oc4ccccc45)n3)c2)c1]

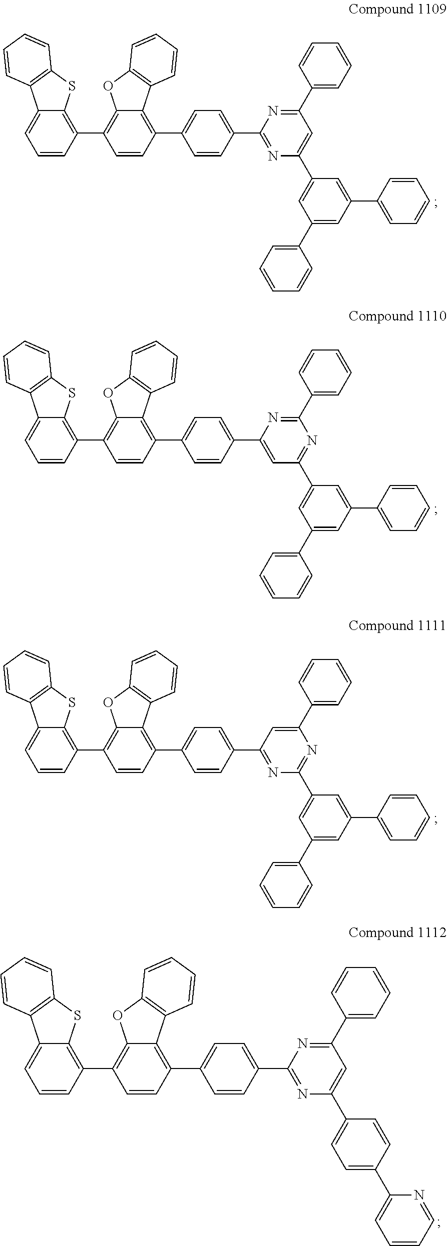 [SMILES: c1ccc(-c2cc(-c3ccccc3)cc(-c3cc(-c4ccccc4)nc(-c4ccc(-c5ccc(-c6cccc7c6sc6ccccc67)c6oc7ccccc7c56)cc4)n3)c2)cc1 ; c1ccc(-c2cc(-c3ccccc3)cc(-c3cc(-c4ccc(-c5ccc(-c6cccc7c6sc6ccccc67)c6oc7ccccc7c56)cc4)nc(-c4ccccc4)n3)c2)cc1 ; c1ccc(-c2cc(-c3ccccc3)cc(-c3nc(-c4ccccc4)cc(-c4ccc(-c5ccc(-c6cccc7c6sc6ccccc67)c6oc7ccccc7c56)cc4)n3)c2)cc1 ; c1ccc(-c2cc(-c3ccc(-c4ccccn4)cc3)nc(-c3ccc(-c4ccc(-c5cccc6c5sc5ccccc56)c5oc6ccccc6c45)cc3)n2)cc1]

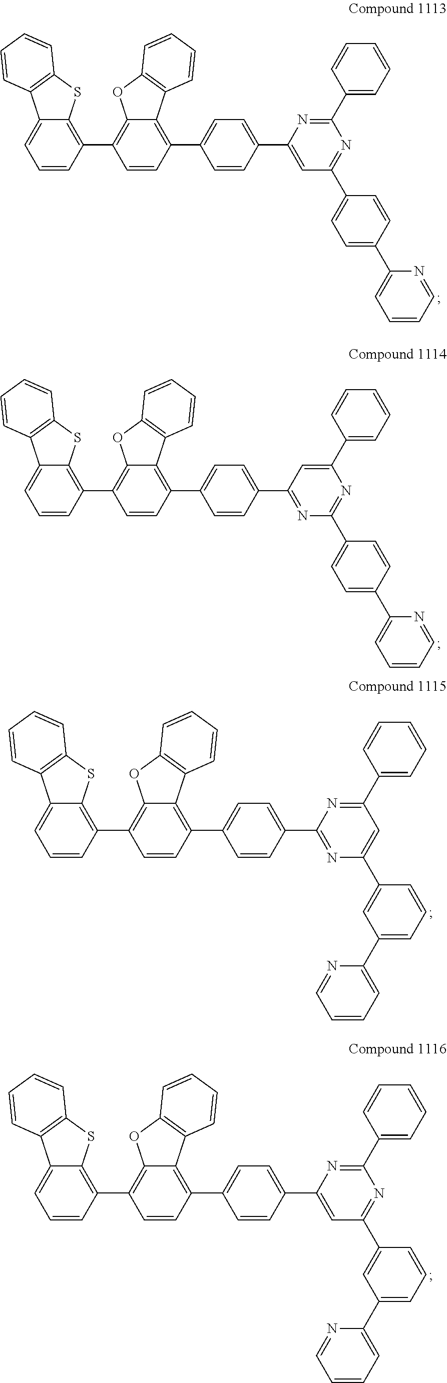 [SMILES: c1ccc(-c2nc(-c3ccc(-c4ccccn4)cc3)cc(-c3ccc(-c4ccc(-c5cccc6c5sc5ccccc56)c5oc6ccccc6c45)cc3)n2)cc1 ; c1ccc(-c2cc(-c3ccc(-c4ccc(-c5cccc6c5sc5ccccc56)c5oc6ccccc6c45)cc3)nc(-c3ccc(-c4ccccn4)cc3)n2)cc1 ; c1ccc(-c2cc(-c3cccc(-c4ccccn4)c3)nc(-c3ccc(-c4ccc(-c5cccc6c5sc5ccccc56)c5oc6ccccc6c45)cc3)n2)cc1 ; c1ccc(-c2nc(-c3ccc(-c4ccc(-c5cccc6c5sc5ccccc56)c5oc6ccccc6c45)cc3)cc(-c3cccc(-c4ccccn4)c3)n2)cc1]

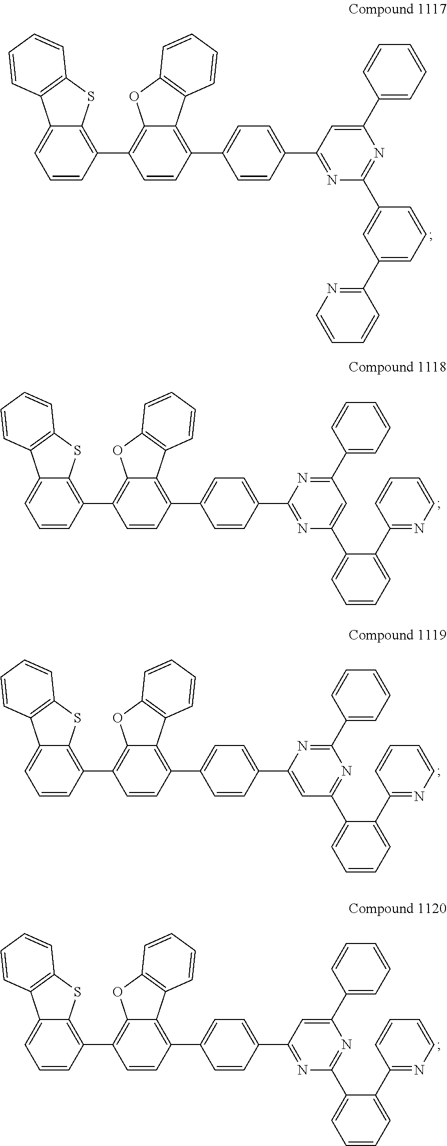 [SMILES: c1ccc(-c2cc(-c3ccc(-c4ccc(-c5cccc6c5sc5ccccc56)c5oc6ccccc6c45)cc3)nc(-c3cccc(-c4ccccn4)c3)n2)cc1 ; c1ccc(-c2cc(-c3ccccc3-c3ccccn3)nc(-c3ccc(-c4ccc(-c5cccc6c5sc5ccccc56)c5oc6ccccc6c45)cc3)n2)cc1 ; c1ccc(-c2nc(-c3ccc(-c4ccc(-c5cccc6c5sc5ccccc56)c5oc6ccccc6c45)cc3)cc(-c3ccccc3-c3ccccn3)n2)cc1 ; c1ccc(-c2cc(-c3ccc(-c4ccc(-c5cccc6c5sc5ccccc56)c5oc6ccccc6c45)cc3)nc(-c3ccccc3-c3ccccn3)n2)cc1]

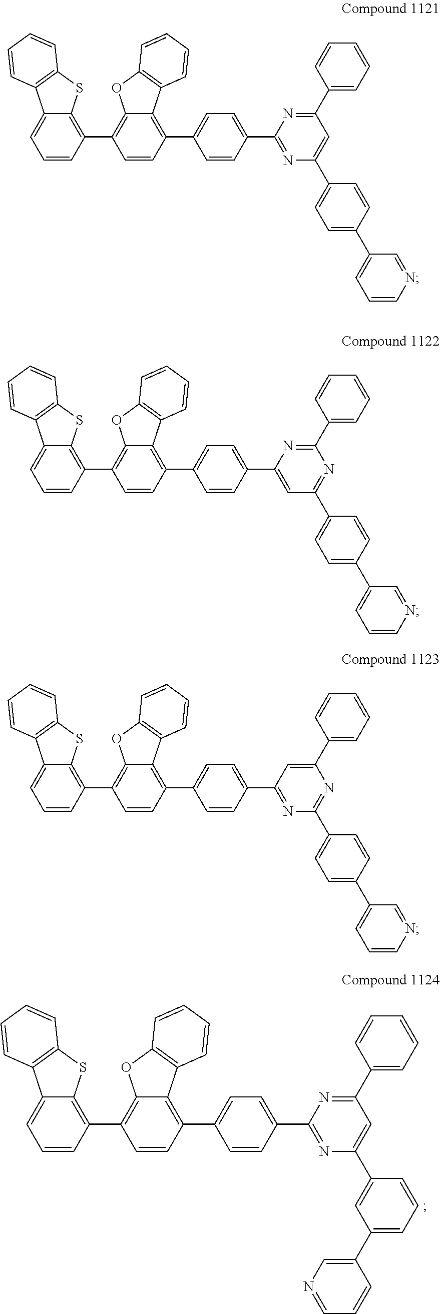 [SMILES: c1ccc(-c2cc(-c3ccc(-c4cccnc4)cc3)nc(-c3ccc(-c4ccc(-c5cccc6c5sc5ccccc56)c5oc6ccccc6c45)cc3)n2)cc1 ; c1ccc(-c2nc(-c3ccc(-c4cccnc4)cc3)cc(-c3ccc(-c4ccc(-c5cccc6c5sc5ccccc56)c5oc6ccccc6c45)cc3)n2)cc1 ; c1ccc(-c2cc(-c3ccc(-c4ccc(-c5cccc6c5sc5ccccc56)c5oc6ccccc6c45)cc3)nc(-c3ccc(-c4cccnc4)cc3)n2)cc1 ; c1ccc(-c2cc(-c3cccc(-c4cccnc4)c3)nc(-c3ccc(-c4ccc(-c5cccc6c5sc5ccccc56)c5oc6ccccc6c45)cc3)n2)cc1]

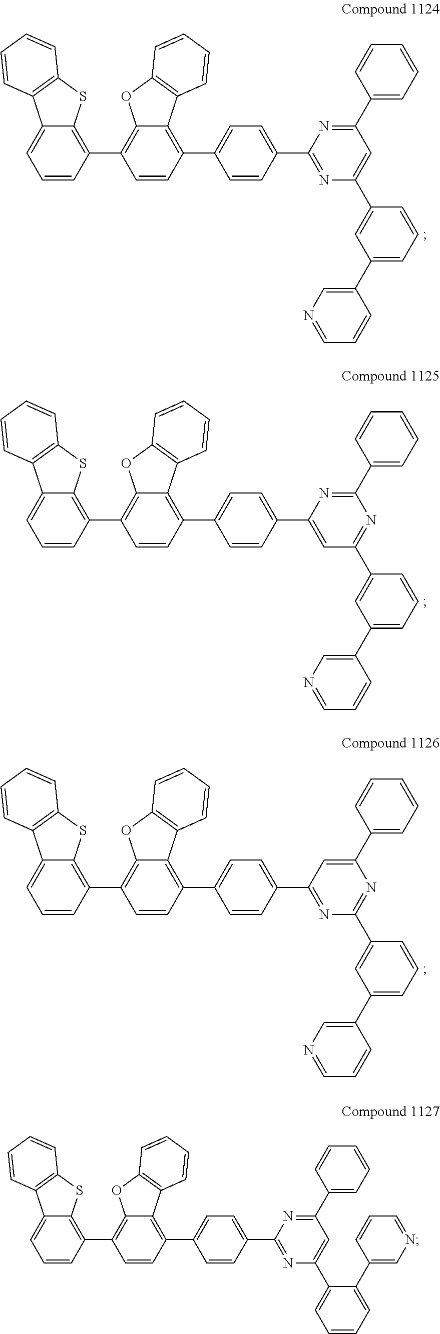 [SMILES: c1ccc(-c2cc(-c3cccc(-c4cccnc4)c3)nc(-c3ccc(-c4ccc(-c5cccc6c5sc5ccccc56)c5oc6ccccc6c45)cc3)n2)cc1 ; c1ccc(-c2nc(-c3ccc(-c4ccc(-c5cccc6c5sc5ccccc56)c5oc6ccccc6c45)cc3)cc(-c3cccc(-c4cccnc4)c3)n2)cc1 ; c1ccc(-c2cc(-c3ccc(-c4ccc(-c5cccc6c5sc5ccccc56)c5oc6ccccc6c45)cc3)nc(-c3cccc(-c4cccnc4)c3)n2)cc1 ; c1ccc(-c2cc(-c3ccccc3-c3cccnc3)nc(-c3ccc(-c4ccc(-c5cccc6c5sc5ccccc56)c5oc6ccccc6c45)cc3)n2)cc1]

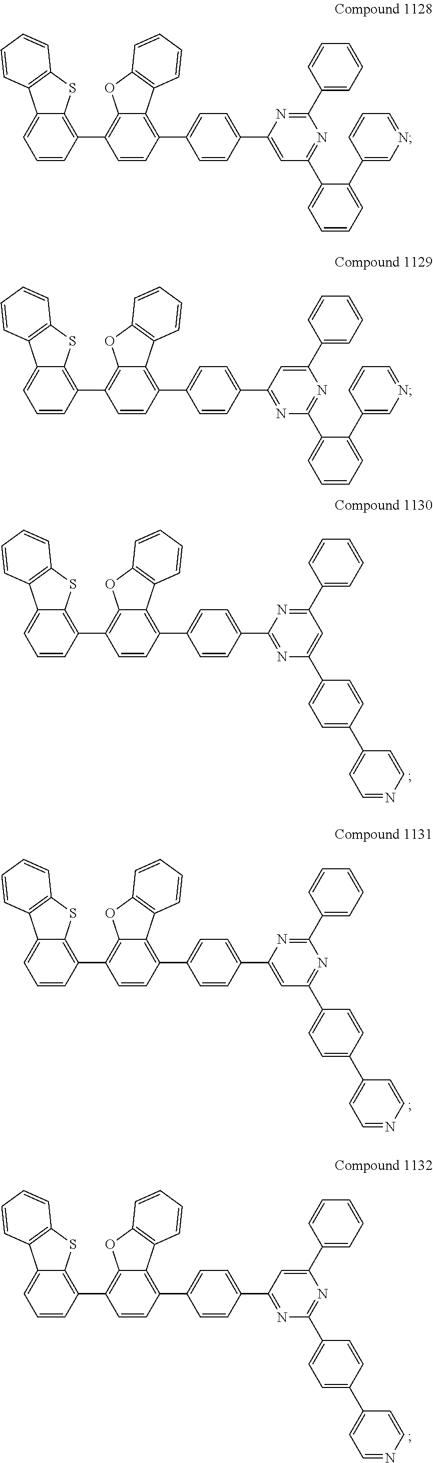 [SMILES: c1ccc(-c2nc(-c3ccc(-c4ccc(-c5cccc6c5sc5ccccc56)c5oc6ccccc6c45)cc3)cc(-c3ccccc3-c3cccnc3)n2)cc1 ; c1ccc(-c2cc(-c3ccc(-c4ccc(-c5cccc6c5sc5ccccc56)c5oc6ccccc6c45)cc3)nc(-c3ccccc3-c3cccnc3)n2)cc1 ; c1ccc(-c2cc(-c3ccc(-c4ccncc4)cc3)nc(-c3ccc(-c4ccc(-c5cccc6c5sc5ccccc56)c5oc6ccccc6c45)cc3)n2)cc1 ; c1ccc(-c2nc(-c3ccc(-c4ccncc4)cc3)cc(-c3ccc(-c4ccc(-c5cccc6c5sc5ccccc56)c5oc6ccccc6c45)cc3)n2)cc1 ; c1ccc(-c2cc(-c3ccc(-c4ccc(-c5cccc6c5sc5ccccc56)c5oc6ccccc6c45)cc3)nc(-c3ccc(-c4ccncc4)cc3)n2)cc1]

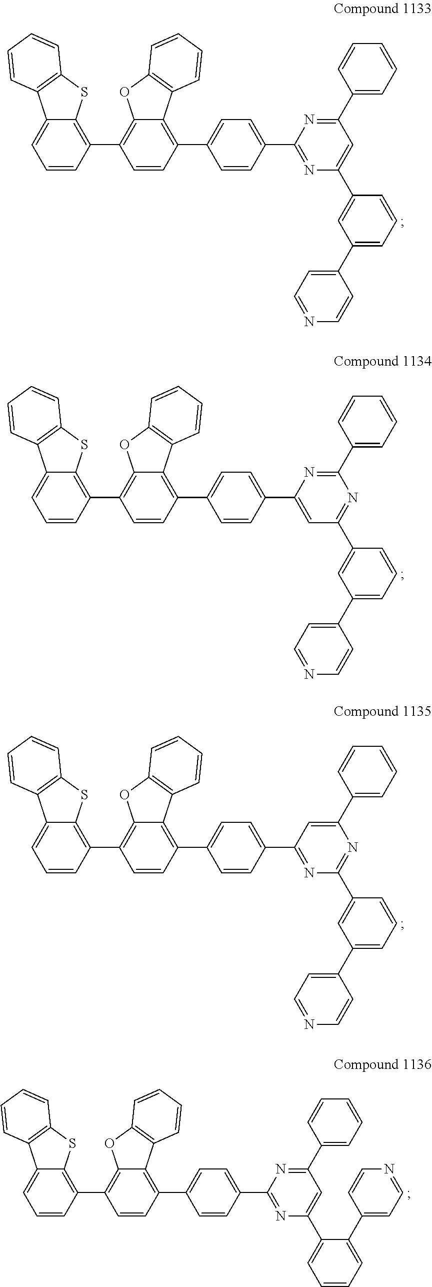 [SMILES: c1ccc(-c2cc(-c3cccc(-c4ccncc4)c3)nc(-c3ccc(-c4ccc(-c5cccc6c5sc5ccccc56)c5oc6ccccc6c45)cc3)n2)cc1 ; c1ccc(-c2nc(-c3ccc(-c4ccc(-c5cccc6c5sc5ccccc56)c5oc6ccccc6c45)cc3)cc(-c3cccc(-c4ccncc4)c3)n2)cc1 ; c1ccc(-c2cc(-c3ccc(-c4ccc(-c5cccc6c5sc5ccccc56)c5oc6ccccc6c45)cc3)nc(-c3cccc(-c4ccncc4)c3)n2)cc1 ; c1ccc(-c2cc(-c3ccccc3-c3ccncc3)nc(-c3ccc(-c4ccc(-c5cccc6c5sc5ccccc56)c5oc6ccccc6c45)cc3)n2)cc1]

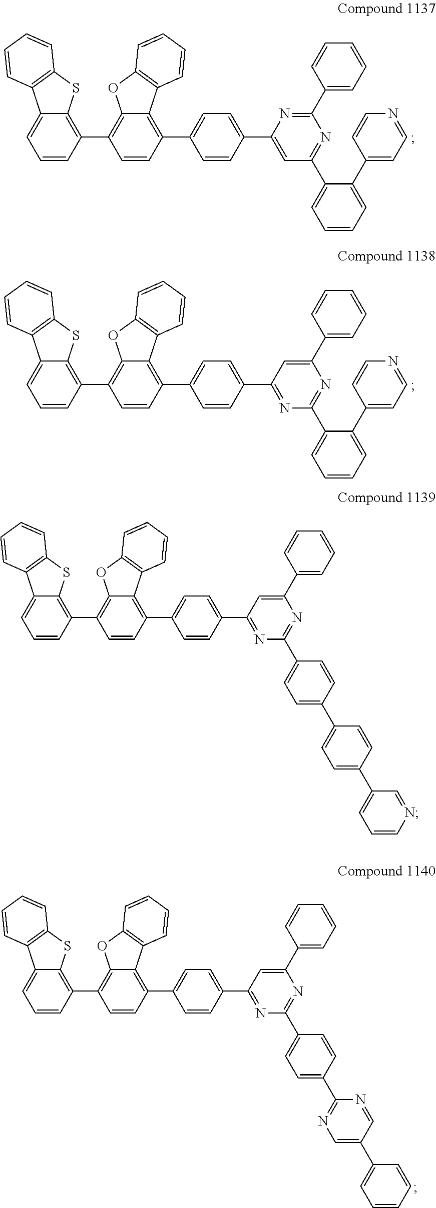 [SMILES: c1ccc(-c2nc(-c3ccc(-c4ccc(-c5cccc6c5sc5ccccc56)c5oc6ccccc6c45)cc3)cc(-c3ccccc3-c3ccncc3)n2)cc1 ; c1ccc(-c2cc(-c3ccc(-c4ccc(-c5cccc6c5sc5ccccc56)c5oc6ccccc6c45)cc3)nc(-c3ccccc3-c3ccncc3)n2)cc1 ; c1ccc(-c2cc(-c3ccc(-c4ccc(-c5cccc6c5sc5ccccc56)c5oc6ccccc6c45)cc3)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc(-c7cccc8c7sc7ccccc78)c7oc8ccccc8c67)cc5)n4)cc3)nc2)cc1]

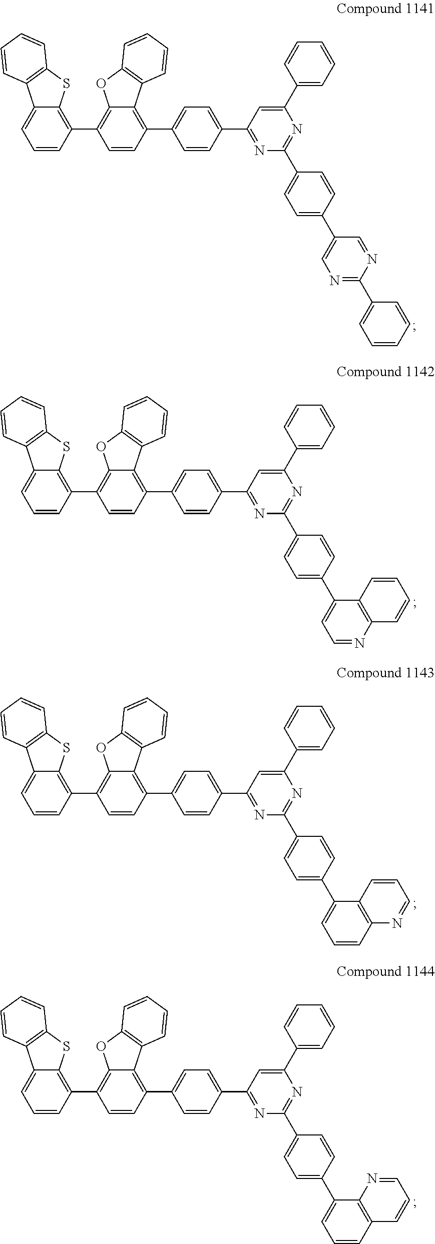 [SMILES: c1ccc(-c2cc(-c3ccc(-c4ccc(-c5cccc6c5sc5ccccc56)c5oc6ccccc6c45)cc3)nc(-c3ccc(-c4cnc(-c5ccccc5)nc4)cc3)n2)cc1 ; c1ccc(-c2cc(-c3ccc(-c4ccc(-c5cccc6c5sc5ccccc56)c5oc6ccccc6c45)cc3)nc(-c3ccc(-c4ccnc5ccccc45)cc3)n2)cc1 ; c1ccc(-c2cc(-c3ccc(-c4ccc(-c5cccc6c5sc5ccccc56)c5oc6ccccc6c45)cc3)nc(-c3ccc(-c4cccc5ncccc45)cc3)n2)cc1 ; c1ccc(-c2cc(-c3ccc(-c4ccc(-c5cccc6c5sc5ccccc56)c5oc6ccccc6c45)cc3)nc(-c3ccc(-c4cccc5cccnc45)cc3)n2)cc1]

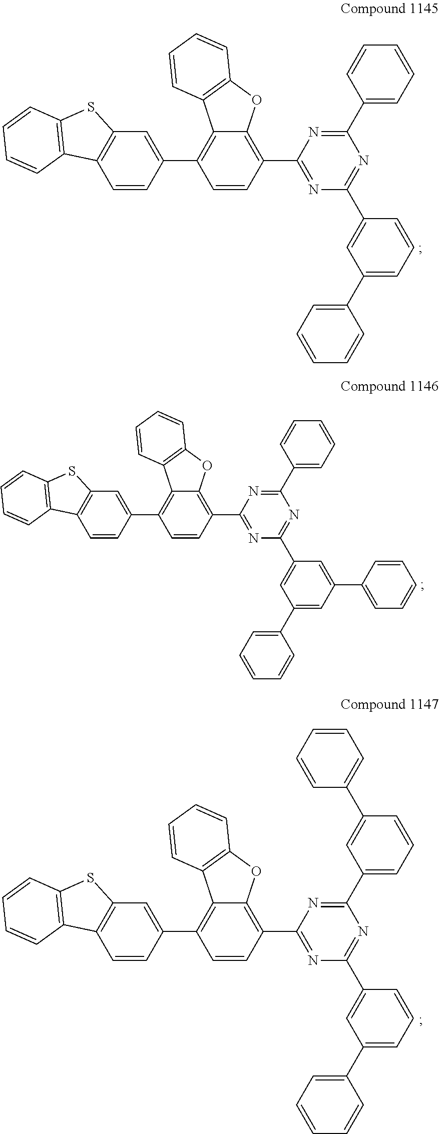 [SMILES: c1ccc(-c2cccc(-c3nc(-c4ccccc4)nc(-c4ccc(-c5ccc6c(c5)sc5ccccc56)c5c4oc4ccccc45)n3)c2)cc1 ; c1ccc(-c2cc(-c3ccccc3)cc(-c3nc(-c4ccccc4)nc(-c4ccc(-c5ccc6c(c5)sc5ccccc56)c5c4oc4ccccc45)n3)c2)cc1 ; c1ccc(-c2cccc(-c3nc(-c4cccc(-c5ccccc5)c4)nc(-c4ccc(-c5ccc6c(c5)sc5ccccc56)c5c4oc4ccccc45)n3)c2)cc1]

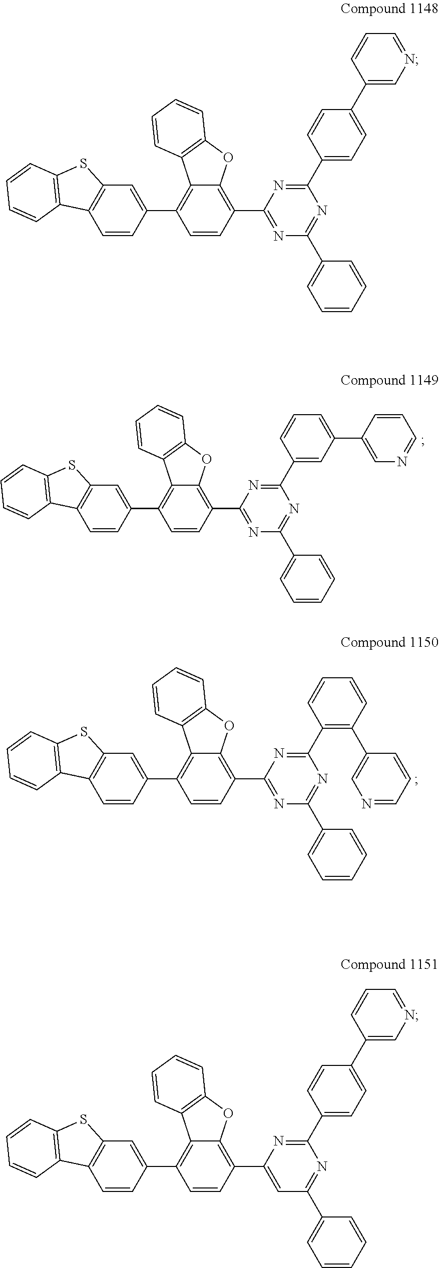 [SMILES: c1ccc(-c2nc(-c3ccc(-c4cccnc4)cc3)nc(-c3ccc(-c4ccc5c(c4)sc4ccccc45)c4c3oc3ccccc34)n2)cc1 ; c1ccc(-c2nc(-c3cccc(-c4cccnc4)c3)nc(-c3ccc(-c4ccc5c(c4)sc4ccccc45)c4c3oc3ccccc34)n2)cc1 ; c1ccc(-c2nc(-c3ccccc3-c3cccnc3)nc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3ccc(-c4cccnc4)cc3)n2)cc1]

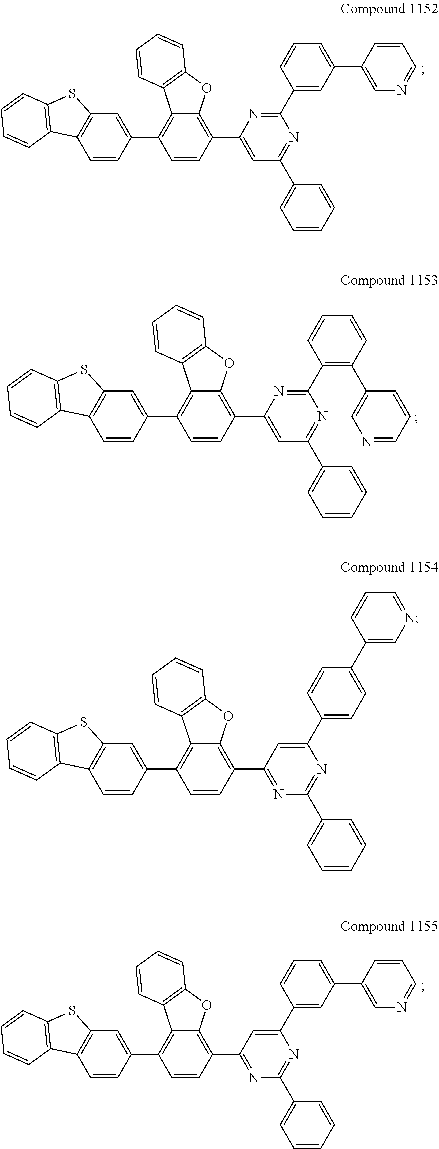 [SMILES: c1ccc(-c2cc(-c3ccc(-c4ccc5c(c4)sc4ccccc45)c4c3oc3ccccc34)nc(-c3cccc(-c4cccnc4)c3)n2)cc1 ; c1ccc(-c2cc(-c3ccc(-c4ccc5c(c4)sc4ccccc45)c4c3oc3ccccc34)nc(-c3ccccc3-c3cccnc3)n2)cc1 ; c1ccc(-c2nc(-c3ccc(-c4cccnc4)cc3)cc(-c3ccc(-c4ccc5c(c4)sc4ccccc45)c4c3oc3ccccc34)n2)cc1 ; c1ccc(-c2nc(-c3cccc(-c4cccnc4)c3)cc(-c3ccc(-c4ccc5c(c4)sc4ccccc45)c4c3oc3ccccc34)n2)cc1]

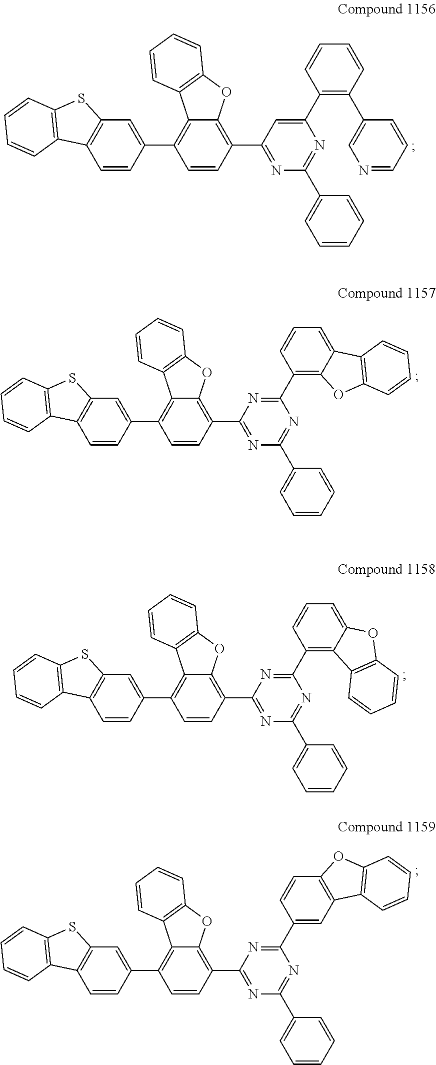 [SMILES: c1ccc(-c2nc(-c3ccccc3-c3cccnc3)cc(-c3ccc(-c4ccc5c(c4)sc4ccccc45)c4c3oc3ccccc34)n2)cc1 ; c1ccc(-c2nc(-c3cccc4c3oc3ccccc34)nc(-c3ccc(-c4ccc5c(c4)sc4ccccc45)c4c3oc3ccccc34)n2)cc1 ; c1ccc(-c2nc(-c3ccc(-c4ccc5c(c4)sc4ccccc45)c4c3oc3ccccc34)nc(-c3cccc4oc5ccccc5c34)n2)cc1 ; c1ccc(-c2nc(-c3ccc4oc5ccccc5c4c3)nc(-c3ccc(-c4ccc5c(c4)sc4ccccc45)c4c3oc3ccccc34)n2)cc1]

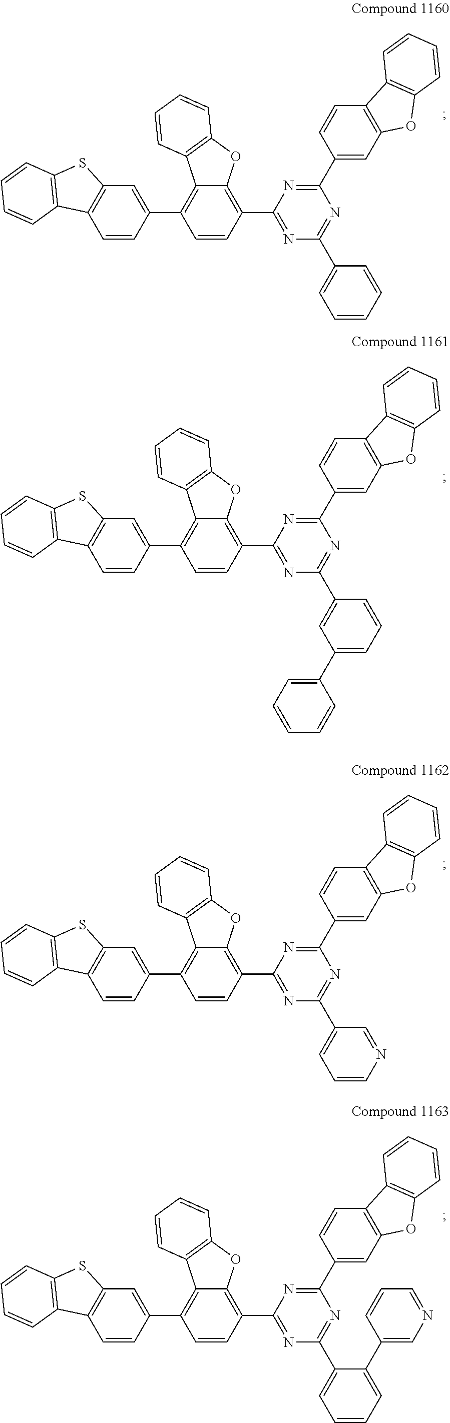 [SMILES: c1ccc(-c2nc(-c3ccc4c(c3)oc3ccccc34)nc(-c3ccc(-c4ccc5c(c4)sc4ccccc45)c4c3oc3ccccc34)n2)cc1 ; c1ccc(-c2cccc(-c3nc(-c4ccc5c(c4)oc4ccccc45)nc(-c4ccc(-c5ccc6c(c5)sc5ccccc56)c5c4oc4ccccc45)n3)c2)cc1 ; c1cncc(-c2nc(-c3ccc4c(c3)oc3ccccc34)nc(-c3ccc(-c4ccc5c(c4)sc4ccccc45)c4c3oc3ccccc34)n2)c1 ; c1cncc(-c2ccccc2-c2nc(-c3ccc4c(c3)oc3ccccc34)nc(-c3ccc(-c4ccc5c(c4)sc4ccccc45)c4c3oc3ccccc34)n2)c1]

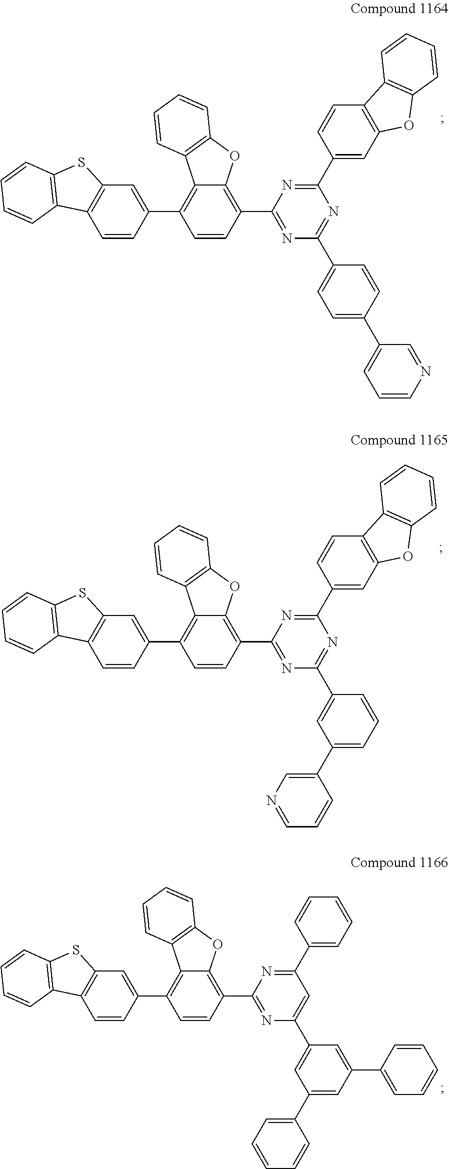 [SMILES: c1cncc(-c2ccc(-c3nc(-c4ccc5c(c4)oc4ccccc45)nc(-c4ccc(-c5ccc6c(c5)sc5ccccc56)c5c4oc4ccccc45)n3)cc2)c1 ; c1cncc(-c2cccc(-c3nc(-c4ccc5c(c4)oc4ccccc45)nc(-c4ccc(-c5ccc6c(c5)sc5ccccc56)c5c4oc4ccccc45)n3)c2)c1 ; c1ccc(-c2cc(-c3ccccc3)cc(-c3cc(-c4ccccc4)nc(-c4ccc(-c5ccc6c(c5)sc5ccccc56)c5c4oc4ccccc45)n3)c2)cc1]

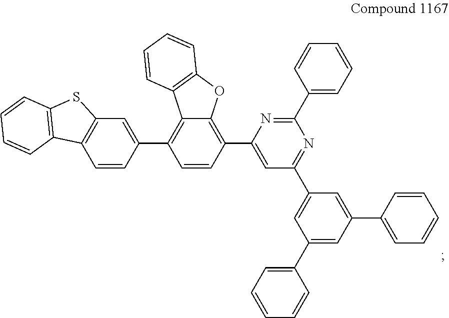 [SMILES: c1ccc(-c2cc(-c3ccccc3)cc(-c3cc(-c4ccc(-c5ccc6c(c5)sc5ccccc56)c5c4oc4ccccc45)nc(-c4ccccc4)n3)c2)cc1]

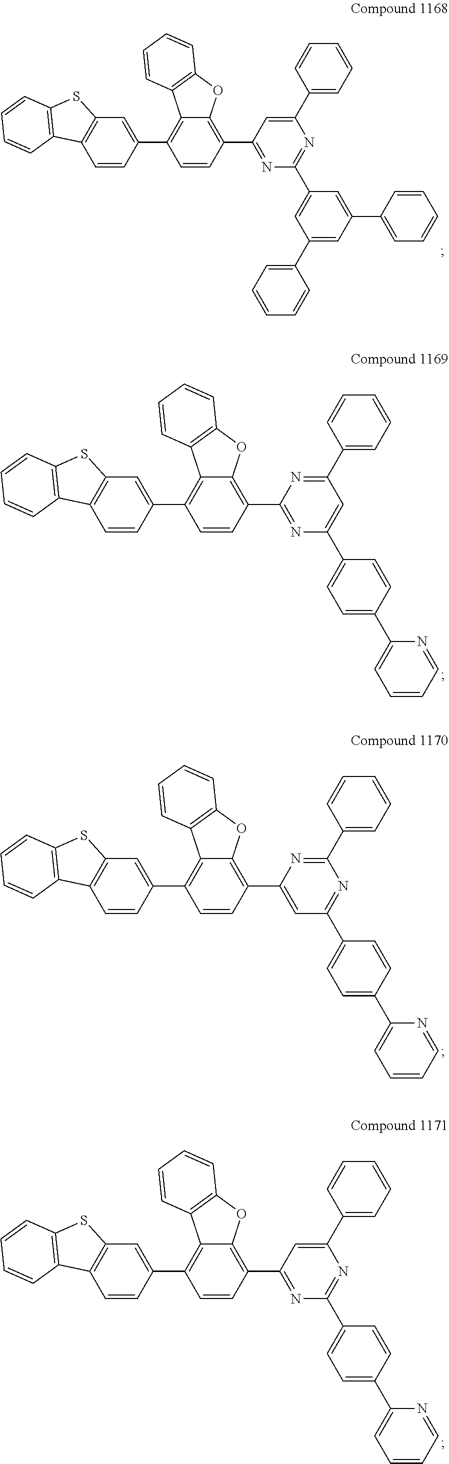 [SMILES: c1ccc(-c2cc(-c3ccccc3)cc(-c3nc(-c4ccccc4)cc(-c4ccc(-c5ccc6c(c5)sc5ccccc56)c5c4oc4ccccc45)n3)c2)cc1 ; c1ccc(-c2cc(-c3ccc(-c4ccccn4)cc3)nc(-c3ccc(-c4ccc5c(c4)sc4ccccc45)c4c3oc3ccccc34)n2)cc1 ; c1ccc(-c2nc(-c3ccc(-c4ccccn4)cc3)cc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3ccc(-c4ccccn4)cc3)n2)cc1]

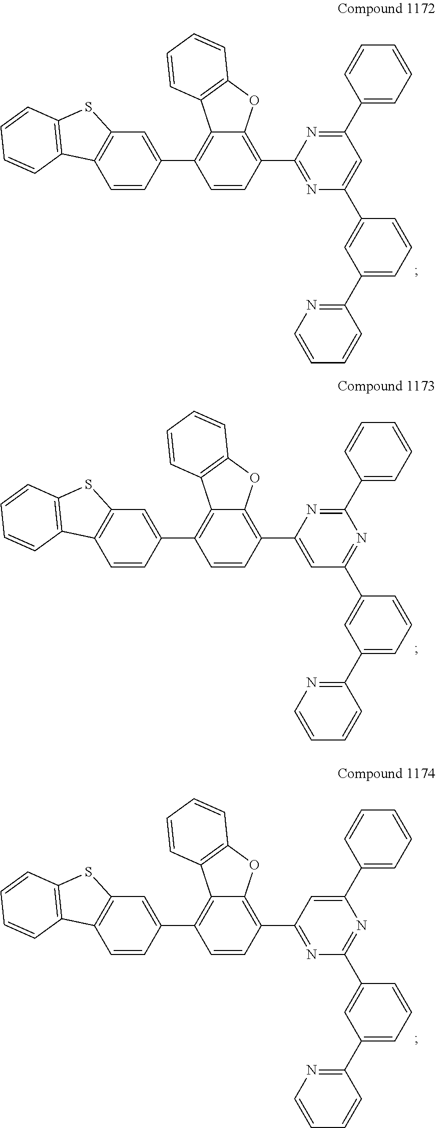 [SMILES: c1ccc(-c2cc(-c3cccc(-c4ccccn4)c3)nc(-c3ccc(-c4ccc5c(c4)sc4ccccc45)c4c3oc3ccccc34)n2)cc1 ; c1ccc(-c2nc(-c3cccc(-c4ccccn4)c3)cc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3cccc(-c4ccccn4)c3)n2)cc1]

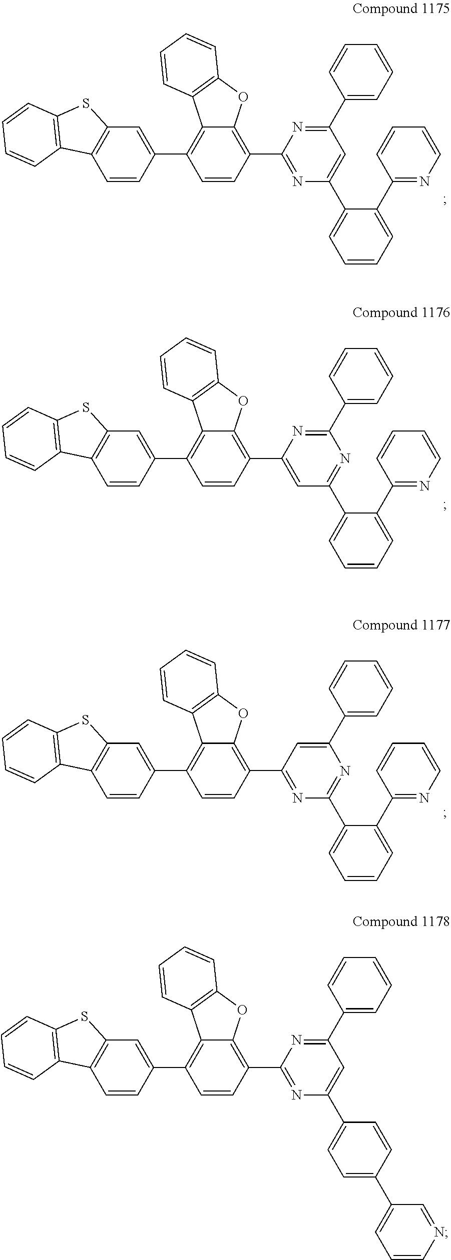 [SMILES: c1ccc(-c2cc(-c3ccccc3-c3ccccn3)nc(-c3ccc(-c4ccc5c(c4)sc4ccccc45)c4c3oc3ccccc34)n2)cc1 ; c1ccc(-c2nc(-c3ccccc3-c3ccccn3)cc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3ccccc3-c3ccccn3)n2)cc1 ; c1ccc(-c2cc(-c3ccc(-c4cccnc4)cc3)nc(-c3ccc(-c4ccc5c(c4)sc4ccccc45)c4c3oc3ccccc34)n2)cc1]

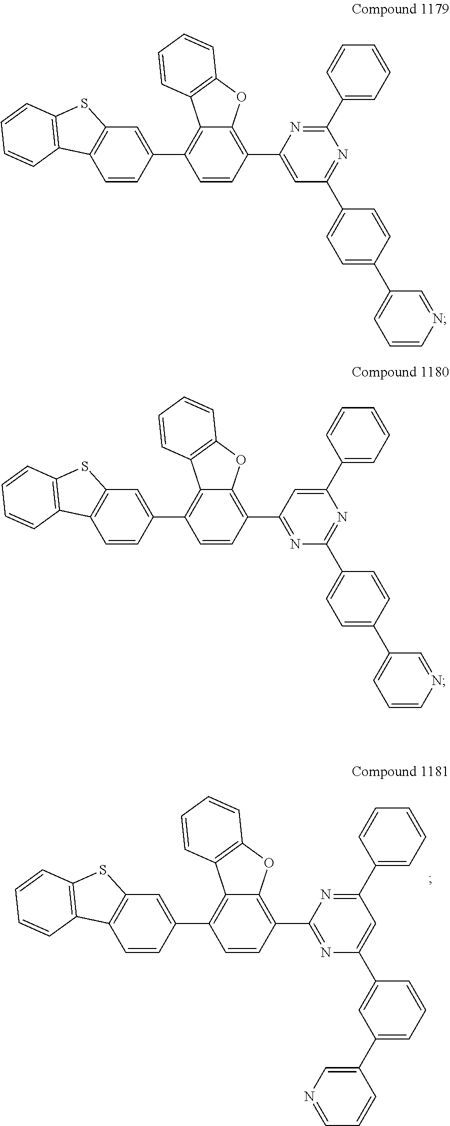 [SMILES: c1ccc(-c2nc(-c3ccc(-c4cccnc4)cc3)cc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3ccc(-c4cccnc4)cc3)n2)cc1 ; c1ccc(-c2cc(-c3cccc(-c4cccnc4)c3)nc(-c3ccc(-c4ccc5c(c4)sc4ccccc45)c4c3oc3ccccc34)n2)cc1]

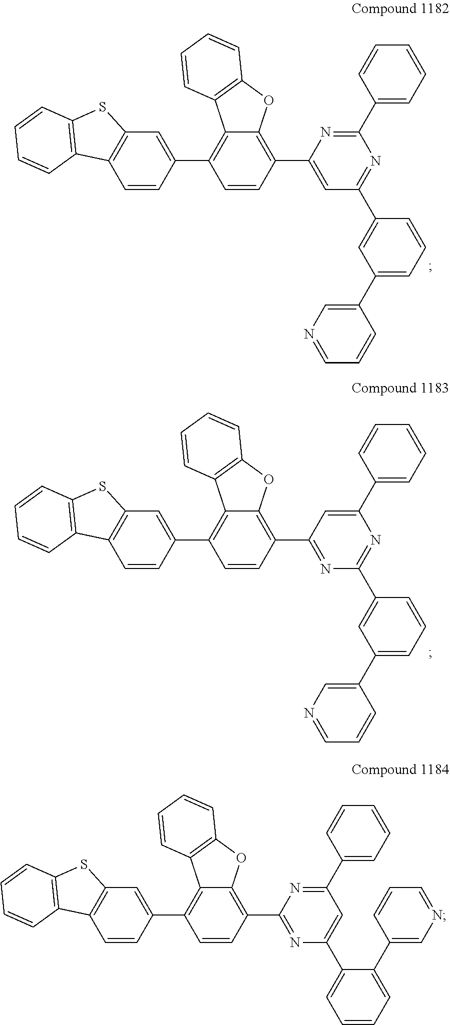 [SMILES: c1ccc(-c2nc(-c3cccc(-c4cccnc4)c3)cc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3cccc(-c4cccnc4)c3)n2)cc1 ; c1ccc(-c2cc(-c3ccccc3-c3cccnc3)nc(-c3ccc(-c4ccc5c(c4)sc4ccccc45)c4c3oc3ccccc34)n2)cc1]

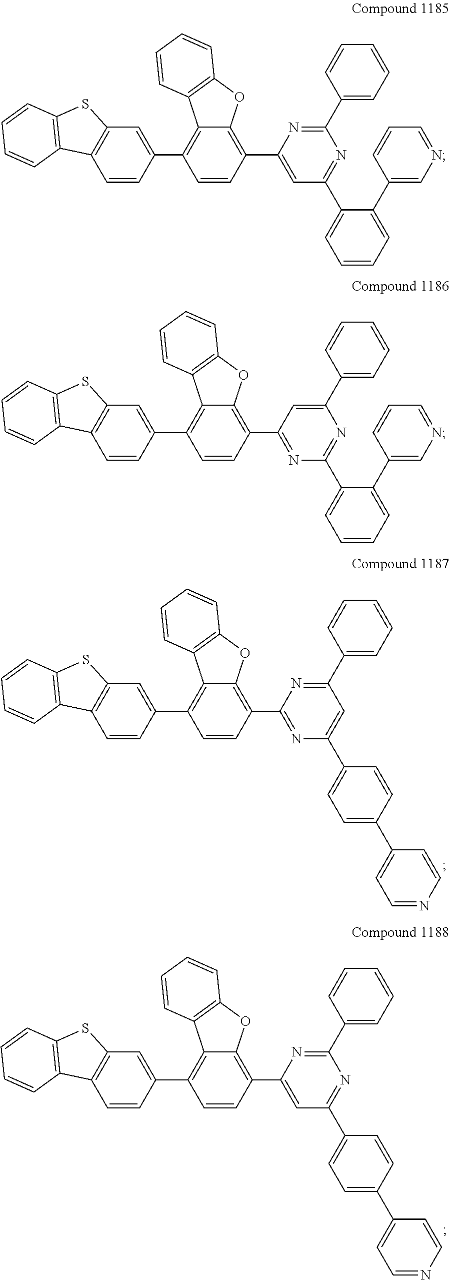 [SMILES: c1ccc(-c2nc(-c3ccccc3-c3cccnc3)cc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3ccccc3-c3cccnc3)n2)cc1 ; c1ccc(-c2cc(-c3ccc(-c4ccncc4)cc3)nc(-c3ccc(-c4ccc5c(c4)sc4ccccc45)c4c3oc3ccccc34)n2)cc1 ; c1ccc(-c2nc(-c3ccc(-c4ccncc4)cc3)cc(-c3ccc(-c4ccc5c(c4)sc4ccccc45)c4c3oc3ccccc34)n2)cc1]

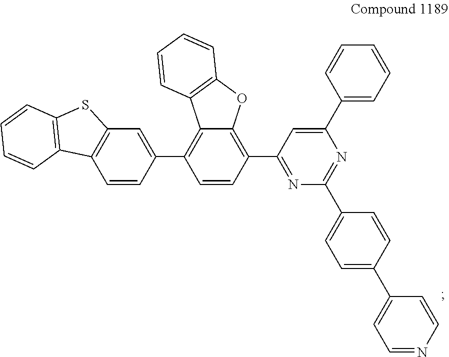 [SMILES: c1ccc(-c2cc(-c3ccc(-c4ccc5c(c4)sc4ccccc45)c4c3oc3ccccc34)nc(-c3ccc(-c4ccncc4)cc3)n2)cc1]

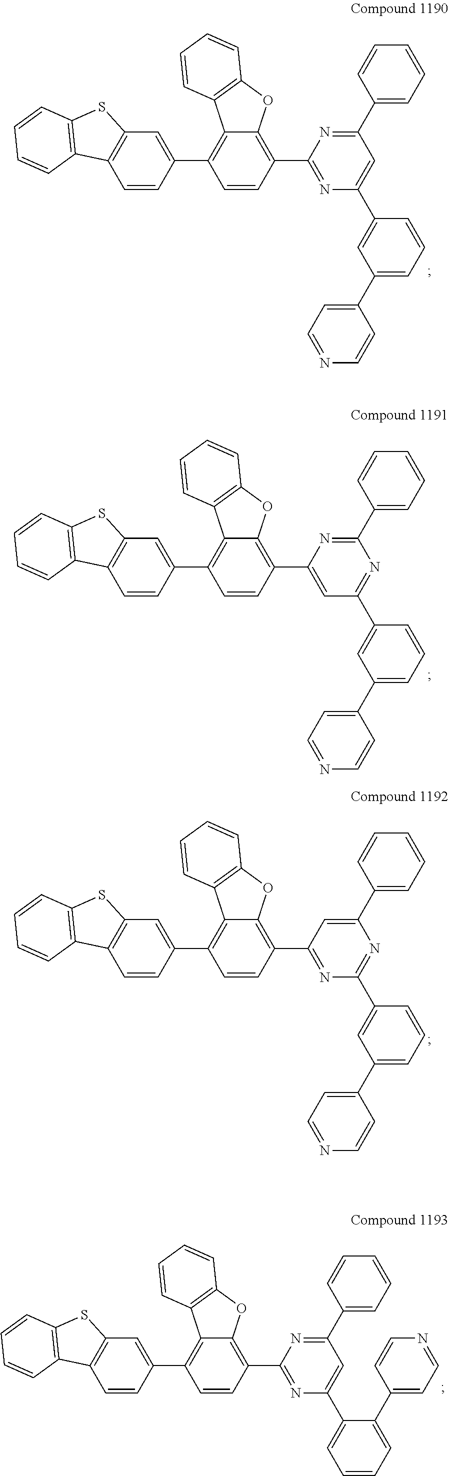 [SMILES: c1ccc(-c2cc(-c3cccc(-c4ccncc4)c3)nc(-c3ccc(-c4ccc5c(c4)sc4ccccc45)c4c3oc3ccccc34)n2)cc1 ; c1ccc(-c2nc(-c3cccc(-c4ccncc4)c3)cc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3cccc(-c4ccncc4)c3)n2)cc1 ; c1ccc(-c2cc(-c3ccccc3-c3ccncc3)nc(-c3ccc(-c4ccc5c(c4)sc4ccccc45)c4c3oc3ccccc34)n2)cc1]

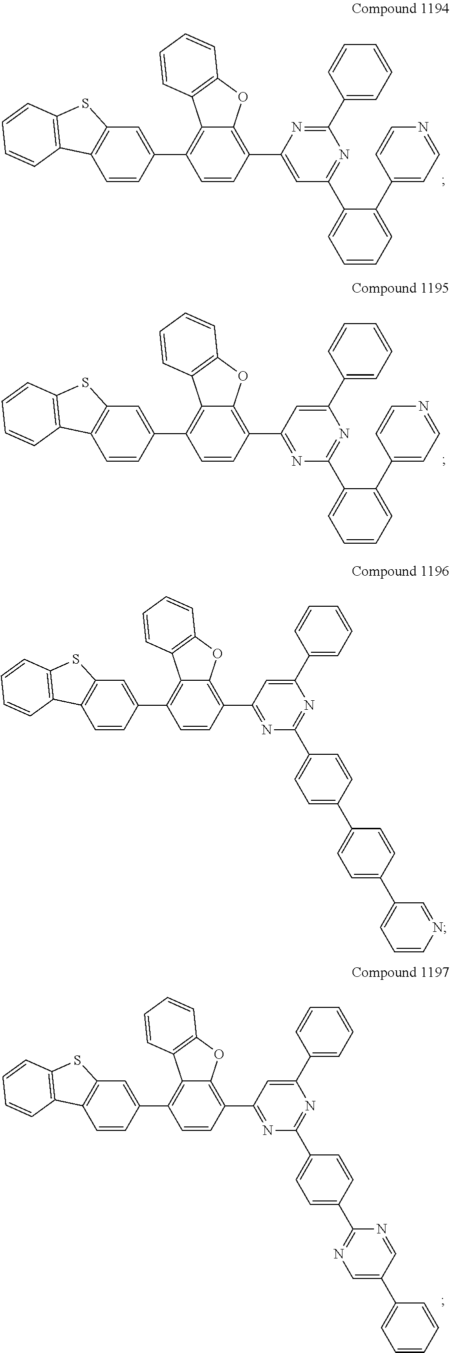 [SMILES: c1ccc(-c2nc(-c3ccccc3-c3ccncc3)cc(-c3ccc(-c4ccc5c(c4)sc4ccccc45)c4c3oc3ccccc34)n2)cc1 ; c1ccc(-c2cc(-c3ccc(-c4ccc5c(c4)sc4ccccc45)c4c3oc3ccccc34)nc(-c3ccccc3-c3ccncc3)n2)cc1 ; c1ccc(-c2cc(-c3ccc(-c4ccc5c(c4)sc4ccccc45)c4c3oc3ccccc34)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc7c(c6)sc6ccccc67)c6c5oc5ccccc56)n4)cc3)nc2)cc1]

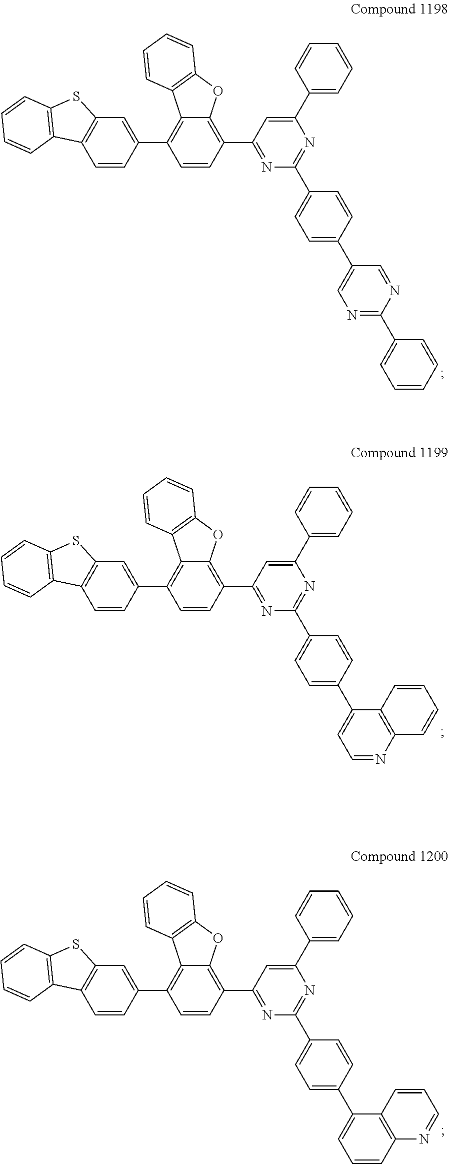 [SMILES: c1ccc(-c2cc(-c3ccc(-c4ccc5c(c4)sc4ccccc45)c4c3oc3ccccc34)nc(-c3ccc(-c4cnc(-c5ccccc5)nc4)cc3)n2)cc1 ; c1ccc(-c2cc(-c3ccc(-c4ccc5c(c4)sc4ccccc45)c4c3oc3ccccc34)nc(-c3ccc(-c4ccnc5ccccc45)cc3)n2)cc1 ; c1ccc(-c2cc(-c3ccc(-c4ccc5c(c4)sc4ccccc45)c4c3oc3ccccc34)nc(-c3ccc(-c4cccc5ncccc45)cc3)n2)cc1]

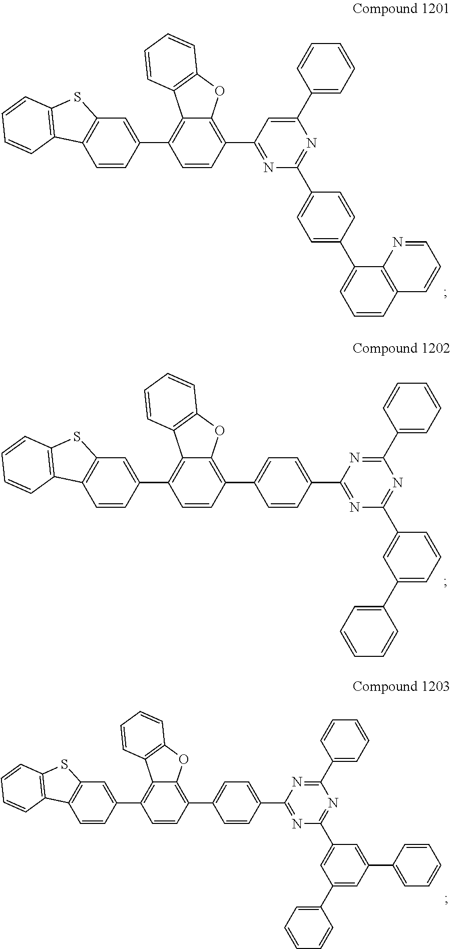 [SMILES: c1ccc(-c2cc(-c3ccc(-c4ccc5c(c4)sc4ccccc45)c4c3oc3ccccc34)nc(-c3ccc(-c4cccc5cccnc45)cc3)n2)cc1 ; c1ccc(-c2cccc(-c3nc(-c4ccccc4)nc(-c4ccc(-c5ccc(-c6ccc7c(c6)sc6ccccc67)c6c5oc5ccccc56)cc4)n3)c2)cc1 ; c1ccc(-c2cc(-c3ccccc3)cc(-c3nc(-c4ccccc4)nc(-c4ccc(-c5ccc(-c6ccc7c(c6)sc6ccccc67)c6c5oc5ccccc56)cc4)n3)c2)cc1]

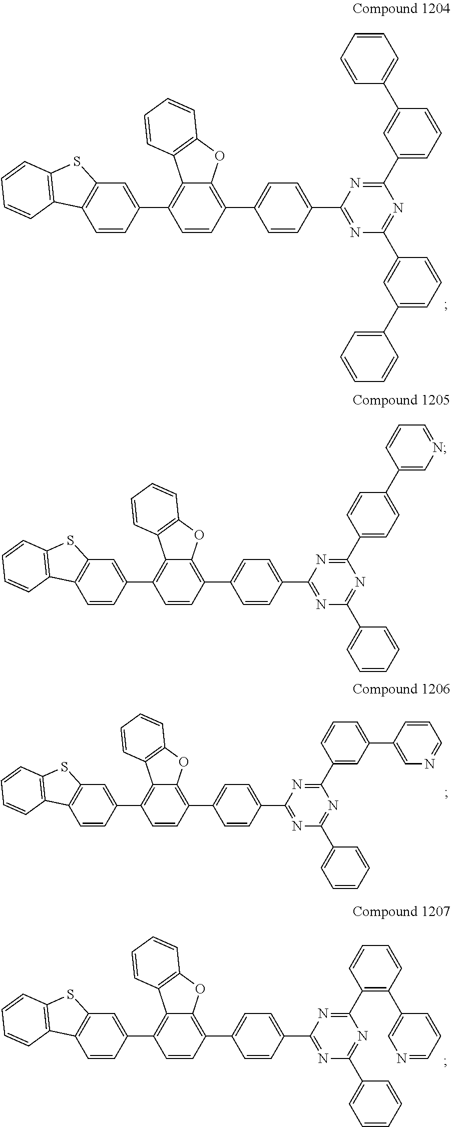 [SMILES: c1ccc(-c2cccc(-c3nc(-c4ccc(-c5ccc(-c6ccc7c(c6)sc6ccccc67)c6c5oc5ccccc56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5ccc6c(c5)sc5ccccc56)c5c4oc4ccccc45)cc3)n2)cc1 ; c1ccc(-c2nc(-c3ccc(-c4ccc(-c5ccc6c(c5)sc5ccccc56)c5c4oc4ccccc45)cc3)nc(-c3cccc(-c4cccnc4)c3)n2)cc1 ; c1ccc(-c2nc(-c3ccc(-c4ccc(-c5ccc6c(c5)sc5ccccc56)c5c4oc4ccccc45)cc3)nc(-c3ccccc3-c3cccnc3)n2)cc1]

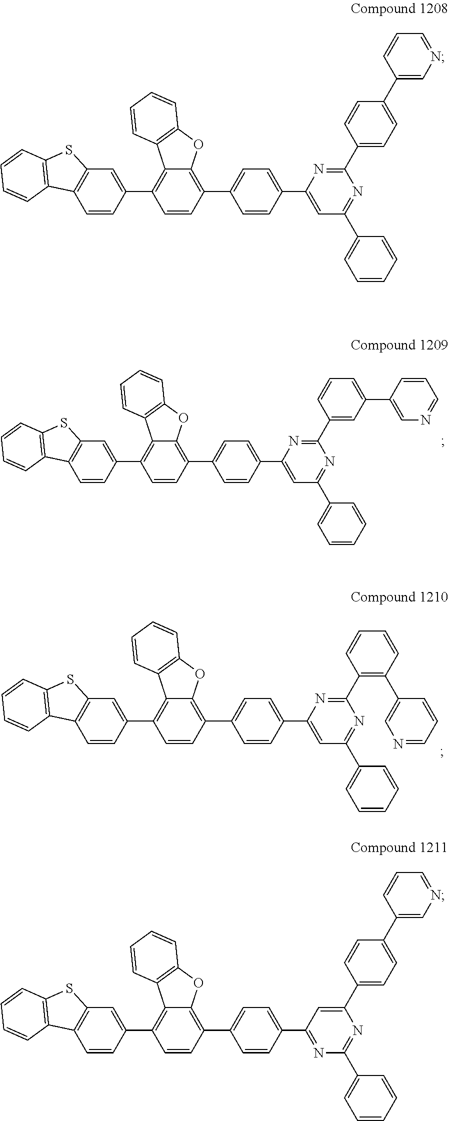 [SMILES: c1ccc(-c2cc(-c3ccc(-c4ccc(-c5ccc6c(c5)sc5ccccc56)c5c4oc4ccccc45)cc3)nc(-c3ccc(-c4cccnc4)cc3)n2)cc1 ; c1ccc(-c2cc(-c3ccc(-c4ccc(-c5ccc6c(c5)sc5ccccc56)c5c4oc4ccccc45)cc3)nc(-c3cccc(-c4cccnc4)c3)n2)cc1 ; c1ccc(-c2cc(-c3ccc(-c4ccc(-c5ccc6c(c5)sc5ccccc56)c5c4oc4ccccc45)cc3)nc(-c3ccccc3-c3cccnc3)n2)cc1 ; c1ccc(-c2nc(-c3ccc(-c4cccnc4)cc3)cc(-c3ccc(-c4ccc(-c5ccc6c(c5)sc5ccccc56)c5c4oc4ccccc45)cc3)n2)cc1]

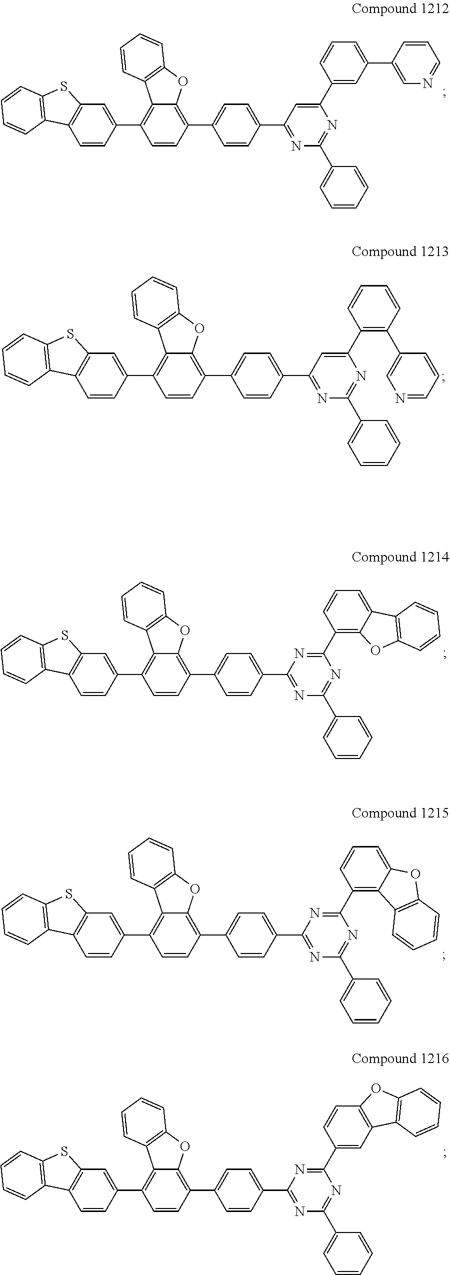 [SMILES: c1ccc(-c2nc(-c3ccc(-c4ccc(-c5ccc6c(c5)sc5ccccc56)c5c4oc4ccccc45)cc3)cc(-c3cccc(-c4cccnc4)c3)n2)cc1 ; c1ccc(-c2nc(-c3ccc(-c4ccc(-c5ccc6c(c5)sc5ccccc56)c5c4oc4ccccc45)cc3)cc(-c3ccccc3-c3cccnc3)n2)cc1 ; c1ccc(-c2nc(-c3ccc(-c4ccc(-c5ccc6c(c5)sc5ccccc56)c5c4oc4ccccc45)cc3)nc(-c3cccc4c3oc3ccccc34)n2)cc1 ; c1ccc(-c2nc(-c3ccc(-c4ccc(-c5ccc6c(c5)sc5ccccc56)c5c4oc4ccccc45)cc3)nc(-c3cccc4oc5ccccc5c34)n2)cc1 ; c1ccc(-c2nc(-c3ccc(-c4ccc(-c5ccc6c(c5)sc5ccccc56)c5c4oc4ccccc45)cc3)nc(-c3ccc4oc5ccccc5c4c3)n2)cc1]

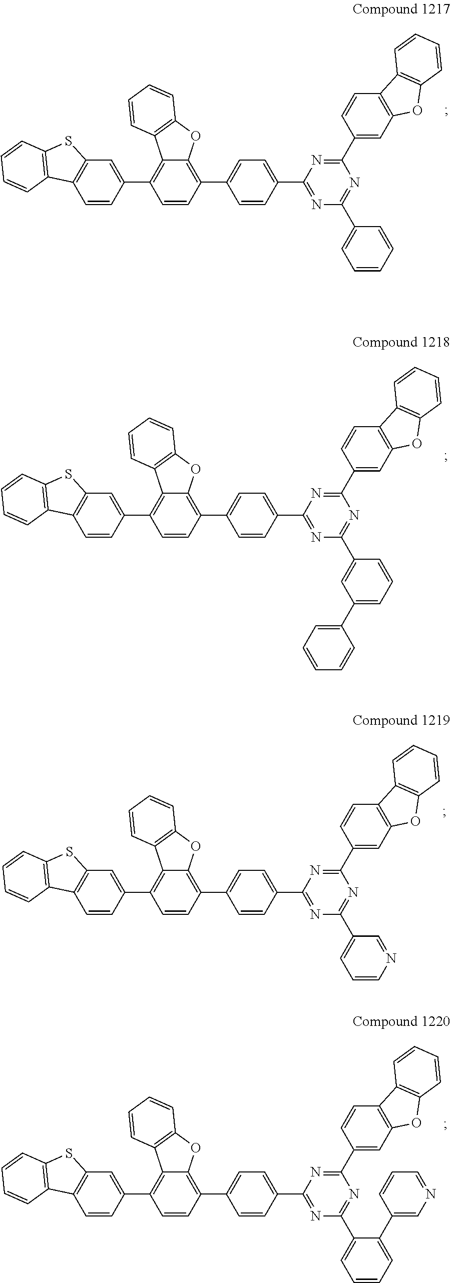 [SMILES: c1ccc(-c2nc(-c3ccc(-c4ccc(-c5ccc6c(c5)sc5ccccc56)c5c4oc4ccccc45)cc3)nc(-c3ccc4c(c3)oc3ccccc34)n2)cc1 ; c1ccc(-c2cccc(-c3nc(-c4ccc(-c5ccc(-c6ccc7c(c6)sc6ccccc67)c6c5oc5ccccc56)cc4)nc(-c4ccc5c(c4)oc4ccccc45)n3)c2)cc1 ; c1cncc(-c2nc(-c3ccc(-c4ccc(-c5ccc6c(c5)sc5ccccc56)c5c4oc4ccccc45)cc3)nc(-c3ccc4c(c3)oc3ccccc34)n2)c1 ; c1cncc(-c2ccccc2-c2nc(-c3ccc(-c4ccc(-c5ccc6c(c5)sc5ccccc56)c5c4oc4ccccc45)cc3)nc(-c3ccc4c(c3)oc3ccccc34)n2)c1]

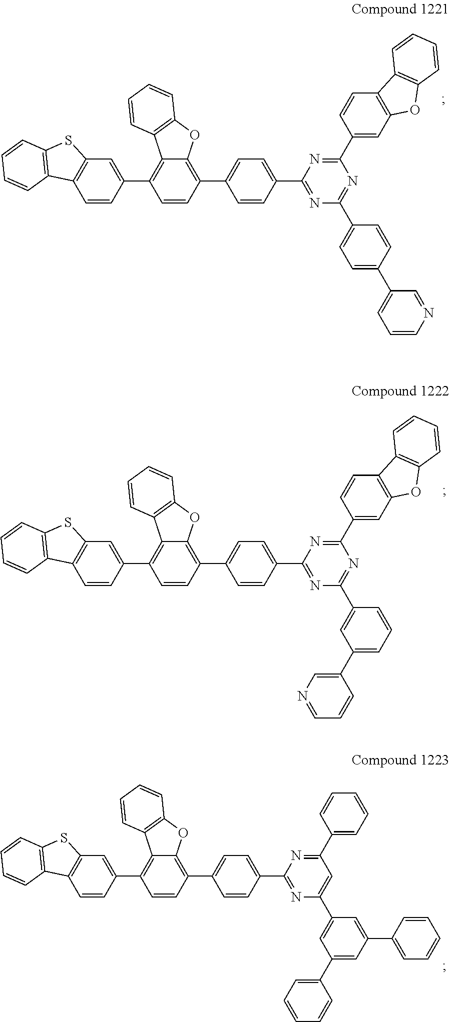 [SMILES: c1cncc(-c2ccc(-c3nc(-c4ccc(-c5ccc(-c6ccc7c(c6)sc6ccccc67)c6c5oc5ccccc56)cc4)nc(-c4ccc5c(c4)oc4ccccc45)n3)cc2)c1 ; c1cncc(-c2cccc(-c3nc(-c4ccc(-c5ccc(-c6ccc7c(c6)sc6ccccc67)c6c5oc5ccccc56)cc4)nc(-c4ccc5c(c4)oc4ccccc45)n3)c2)c1 ; c1ccc(-c2cc(-c3ccccc3)cc(-c3cc(-c4ccccc4)nc(-c4ccc(-c5ccc(-c6ccc7c(c6)sc6ccccc67)c6c5oc5ccccc56)cc4)n3)c2)cc1]

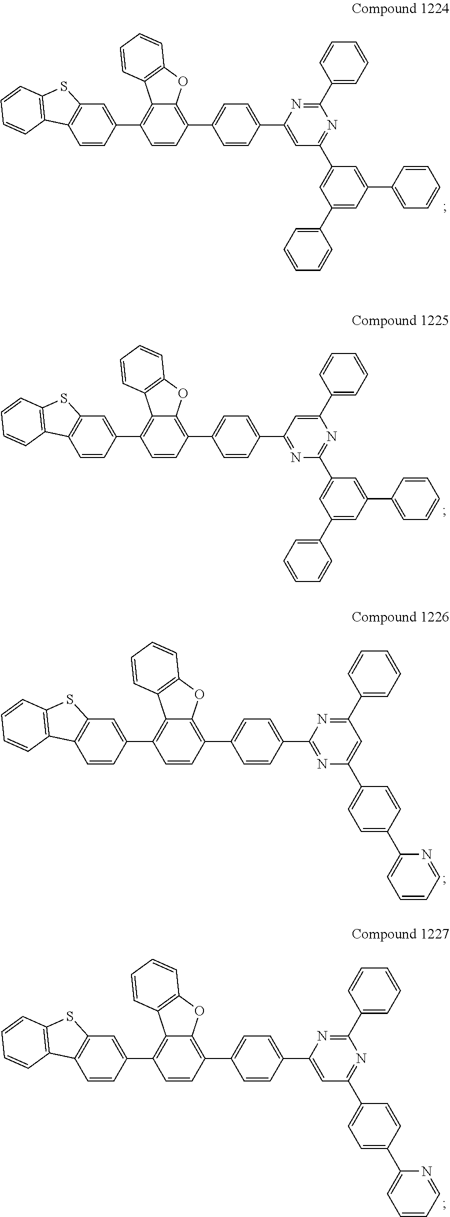 [SMILES: c1ccc(-c2cc(-c3ccccc3)cc(-c3cc(-c4ccc(-c5ccc(-c6ccc7c(c6)sc6ccccc67)c6c5oc5ccccc56)cc4)nc(-c4ccccc4)n3)c2)cc1 ; c1ccc(-c2cc(-c3ccccc3)cc(-c3nc(-c4ccccc4)cc(-c4ccc(-c5ccc(-c6ccc7c(c6)sc6ccccc67)c6c5oc5ccccc56)cc4)n3)c2)cc1 ; c1ccc(-c2cc(-c3ccc(-c4ccccn4)cc3)nc(-c3ccc(-c4ccc(-c5ccc6c(c5)sc5ccccc56)c5c4oc4ccccc45)cc3)n2)cc1 ; c1ccc(-c2nc(-c3ccc(-c4ccccn4)cc3)cc(-c3ccc(-c4ccc(-c5ccc6c(c5)sc5ccccc56)c5c4oc4ccccc45)cc3)n2)cc1]

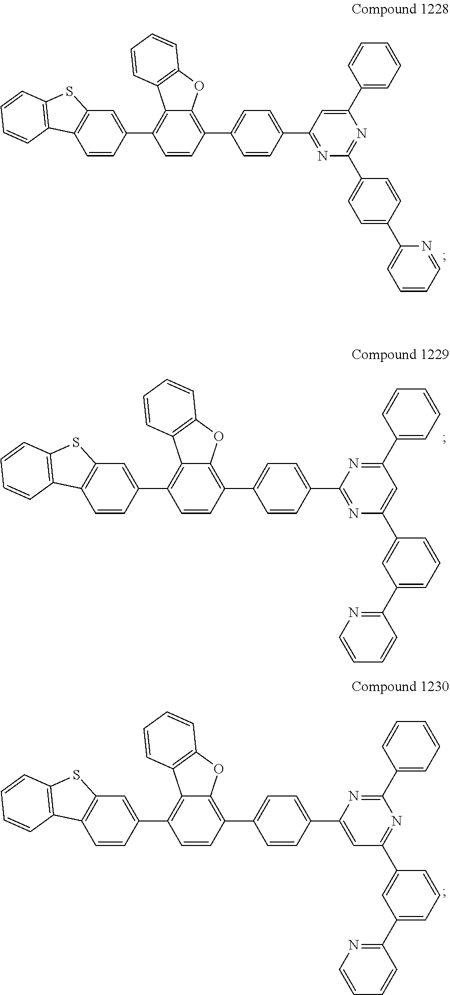 [SMILES: c1ccc(-c2cc(-c3ccc(-c4ccc(-c5ccc6c(c5)sc5ccccc56)c5c4oc4ccccc45)cc3)nc(-c3ccc(-c4ccccn4)cc3)n2)cc1 ; c1ccc(-c2cc(-c3cccc(-c4ccccn4)c3)nc(-c3ccc(-c4ccc(-c5ccc6c(c5)sc5ccccc56)c5c4oc4ccccc45)cc3)n2)cc1 ; c1ccc(-c2nc(-c3ccc(-c4ccc(-c5ccc6c(c5)sc5ccccc56)c5c4oc4ccccc45)cc3)cc(-c3cccc(-c4ccccn4)c3)n2)cc1]

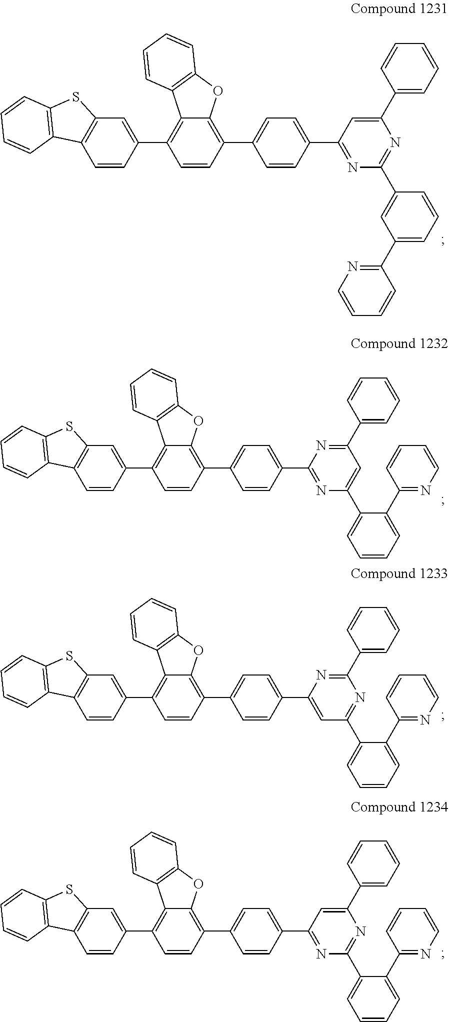 [SMILES: c1ccc(-c2cc(-c3ccc(-c4ccc(-c5ccc6c(c5)sc5ccccc56)c5c4oc4ccccc45)cc3)nc(-c3cccc(-c4ccccn4)c3)n2)cc1 ; c1ccc(-c2cc(-c3ccccc3-c3ccccn3)nc(-c3ccc(-c4ccc(-c5ccc6c(c5)sc5ccccc56)c5c4oc4ccccc45)cc3)n2)cc1 ; c1ccc(-c2nc(-c3ccc(-c4ccc(-c5ccc6c(c5)sc5ccccc56)c5c4oc4ccccc45)cc3)cc(-c3ccccc3-c3ccccn3)n2)cc1 ; c1ccc(-c2cc(-c3ccc(-c4ccc(-c5ccc6c(c5)sc5ccccc56)c5c4oc4ccccc45)cc3)nc(-c3ccccc3-c3ccccn3)n2)cc1]

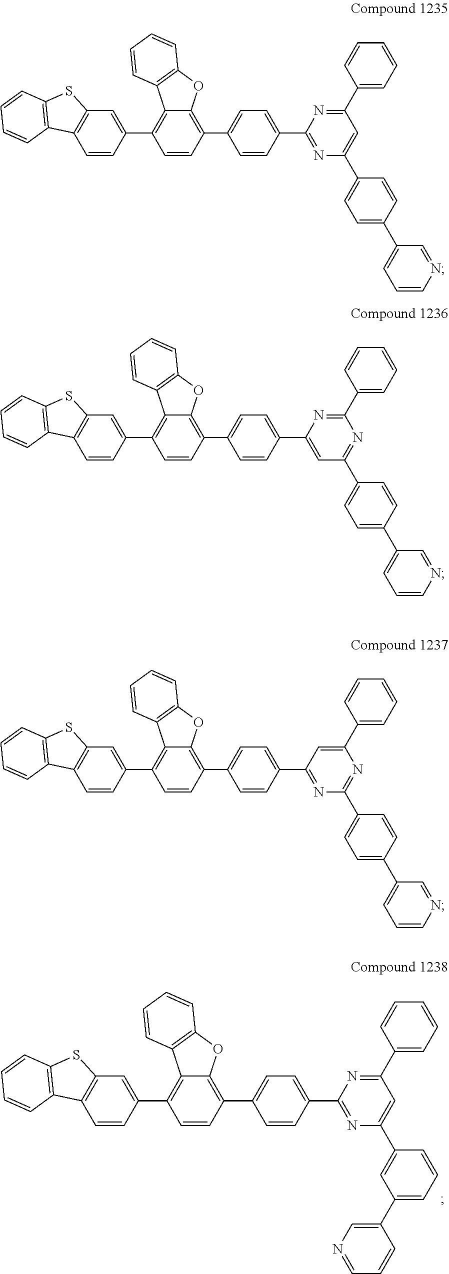 [SMILES: c1ccc(-c2cc(-c3ccc(-c4cccnc4)cc3)nc(-c3ccc(-c4ccc(-c5ccc6c(c5)sc5ccccc56)c5c4oc4ccccc45)cc3)n2)cc1 ; c1ccc(-c2nc(-c3ccc(-c4cccnc4)cc3)cc(-c3ccc(-c4ccc(-c5ccc6c(c5)sc5ccccc56)c5c4oc4ccccc45)cc3)n2)cc1 ; c1ccc(-c2cc(-c3ccc(-c4ccc(-c5ccc6c(c5)sc5ccccc56)c5c4oc4ccccc45)cc3)nc(-c3ccc(-c4cccnc4)cc3)n2)cc1 ; c1ccc(-c2cc(-c3cccc(-c4cccnc4)c3)nc(-c3ccc(-c4ccc(-c5ccc6c(c5)sc5ccccc56)c5c4oc4ccccc45)cc3)n2)cc1]

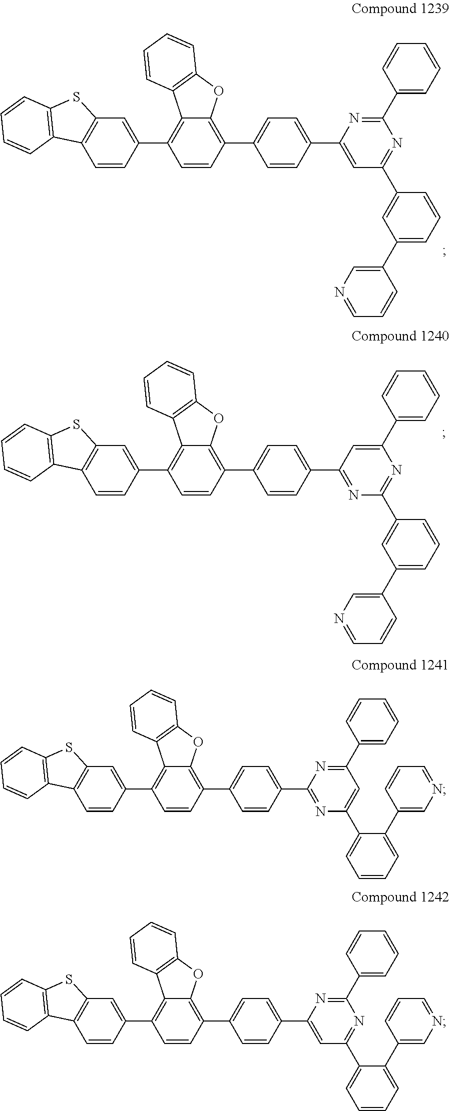 [SMILES: c1ccc(-c2nc(-c3ccc(-c4ccc(-c5ccc6c(c5)sc5ccccc56)c5c4oc4ccccc45)cc3)cc(-c3cccc(-c4cccnc4)c3)n2)cc1 ; c1ccc(-c2cc(-c3ccc(-c4ccc(-c5ccc6c(c5)sc5ccccc56)c5c4oc4ccccc45)cc3)nc(-c3cccc(-c4cccnc4)c3)n2)cc1 ; c1ccc(-c2cc(-c3ccccc3-c3cccnc3)nc(-c3ccc(-c4ccc(-c5ccc6c(c5)sc5ccccc56)c5c4oc4ccccc45)cc3)n2)cc1 ; c1ccc(-c2nc(-c3ccc(-c4ccc(-c5ccc6c(c5)sc5ccccc56)c5c4oc4ccccc45)cc3)cc(-c3ccccc3-c3cccnc3)n2)cc1]

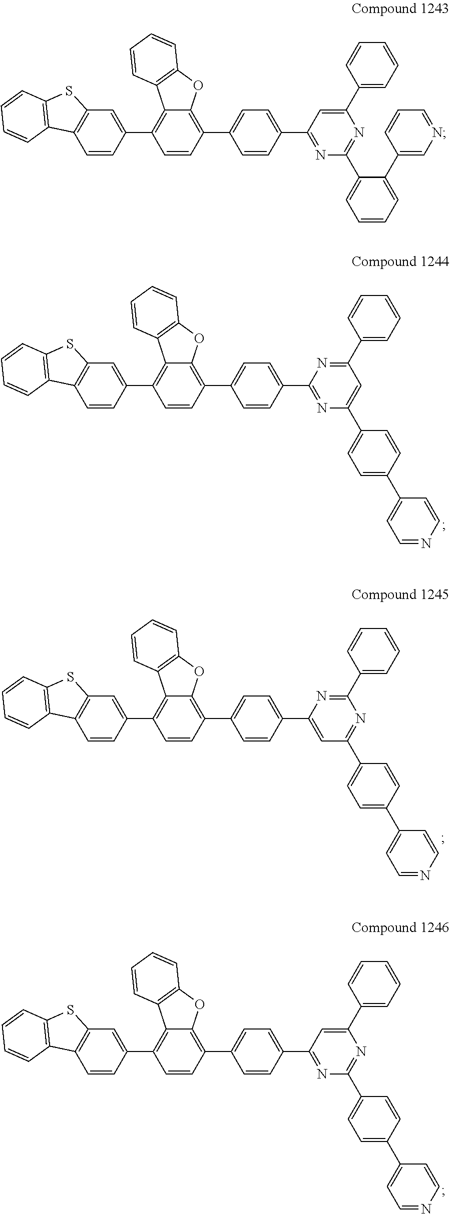 [SMILES: c1ccc(-c2cc(-c3ccc(-c4ccc(-c5ccc6c(c5)sc5ccccc56)c5c4oc4ccccc45)cc3)nc(-c3ccccc3-c3cccnc3)n2)cc1 ; c1ccc(-c2cc(-c3ccc(-c4ccncc4)cc3)nc(-c3ccc(-c4ccc(-c5ccc6c(c5)sc5ccccc56)c5c4oc4ccccc45)cc3)n2)cc1 ; c1ccc(-c2nc(-c3ccc(-c4ccncc4)cc3)cc(-c3ccc(-c4ccc(-c5ccc6c(c5)sc5ccccc56)c5c4oc4ccccc45)cc3)n2)cc1 ; c1ccc(-c2cc(-c3ccc(-c4ccc(-c5ccc6c(c5)sc5ccccc56)c5c4oc4ccccc45)cc3)nc(-c3ccc(-c4ccncc4)cc3)n2)cc1]

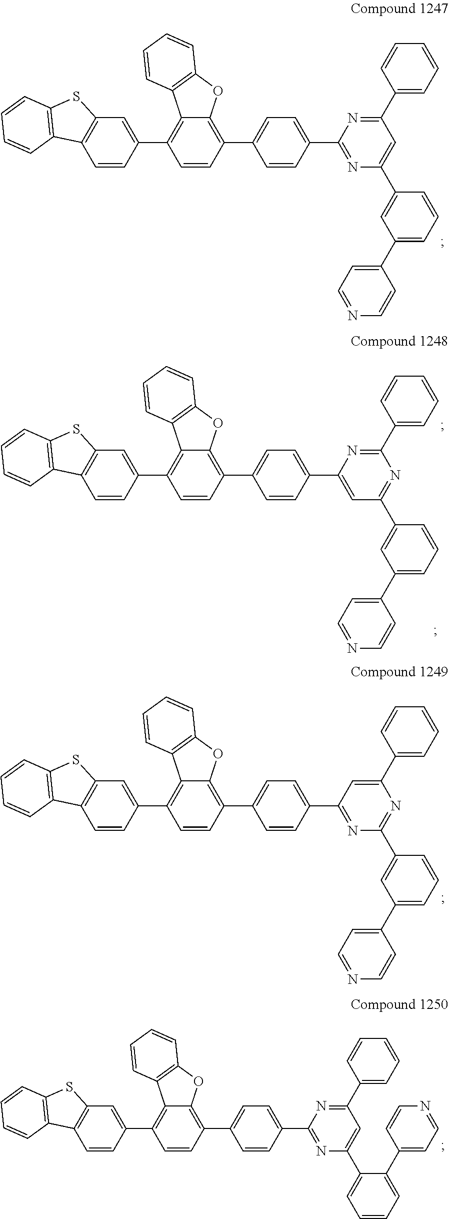 [SMILES: c1ccc(-c2cc(-c3cccc(-c4ccncc4)c3)nc(-c3ccc(-c4ccc(-c5ccc6c(c5)sc5ccccc56)c5c4oc4ccccc45)cc3)n2)cc1 ; c1ccc(-c2nc(-c3ccc(-c4ccc(-c5ccc6c(c5)sc5ccccc56)c5c4oc4ccccc45)cc3)cc(-c3cccc(-c4ccncc4)c3)n2)cc1 ; c1ccc(-c2cc(-c3ccc(-c4ccc(-c5ccc6c(c5)sc5ccccc56)c5c4oc4ccccc45)cc3)nc(-c3cccc(-c4ccncc4)c3)n2)cc1 ; c1ccc(-c2cc(-c3ccccc3-c3ccncc3)nc(-c3ccc(-c4ccc(-c5ccc6c(c5)sc5ccccc56)c5c4oc4ccccc45)cc3)n2)cc1]

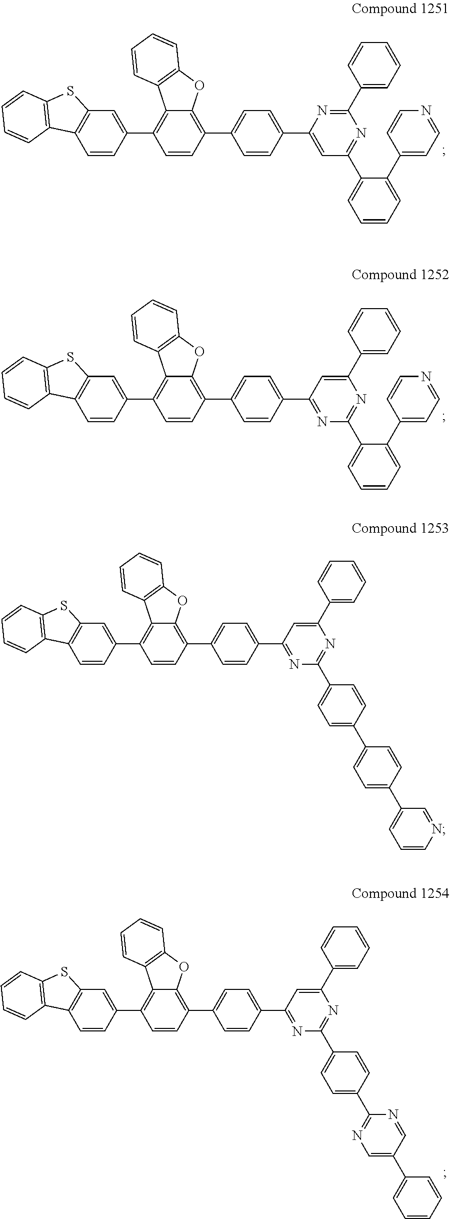 [SMILES: c1ccc(-c2nc(-c3ccc(-c4ccc(-c5ccc6c(c5)sc5ccccc56)c5c4oc4ccccc45)cc3)cc(-c3ccccc3-c3ccncc3)n2)cc1 ; c1ccc(-c2cc(-c3ccc(-c4ccc(-c5ccc6c(c5)sc5ccccc56)c5c4oc4ccccc45)cc3)nc(-c3ccccc3-c3ccncc3)n2)cc1 ; c1ccc(-c2cc(-c3ccc(-c4ccc(-c5ccc6c(c5)sc5ccccc56)c5c4oc4ccccc45)cc3)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc(-c7ccc8c(c7)sc7ccccc78)c7c6oc6ccccc67)cc5)n4)cc3)nc2)cc1]

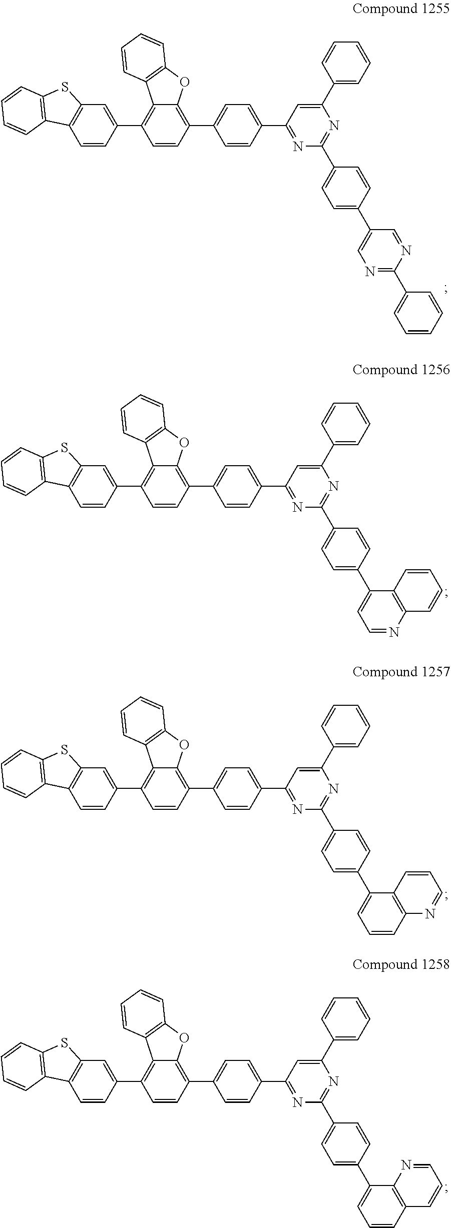 [SMILES: c1ccc(-c2cc(-c3ccc(-c4ccc(-c5ccc6c(c5)sc5ccccc56)c5c4oc4ccccc45)cc3)nc(-c3ccc(-c4cnc(-c5ccccc5)nc4)cc3)n2)cc1 ; c1ccc(-c2cc(-c3ccc(-c4ccc(-c5ccc6c(c5)sc5ccccc56)c5c4oc4ccccc45)cc3)nc(-c3ccc(-c4ccnc5ccccc45)cc3)n2)cc1 ; c1ccc(-c2cc(-c3ccc(-c4ccc(-c5ccc6c(c5)sc5ccccc56)c5c4oc4ccccc45)cc3)nc(-c3ccc(-c4cccc5ncccc45)cc3)n2)cc1 ; c1ccc(-c2cc(-c3ccc(-c4ccc(-c5ccc6c(c5)sc5ccccc56)c5c4oc4ccccc45)cc3)nc(-c3ccc(-c4cccc5cccnc45)cc3)n2)cc1]

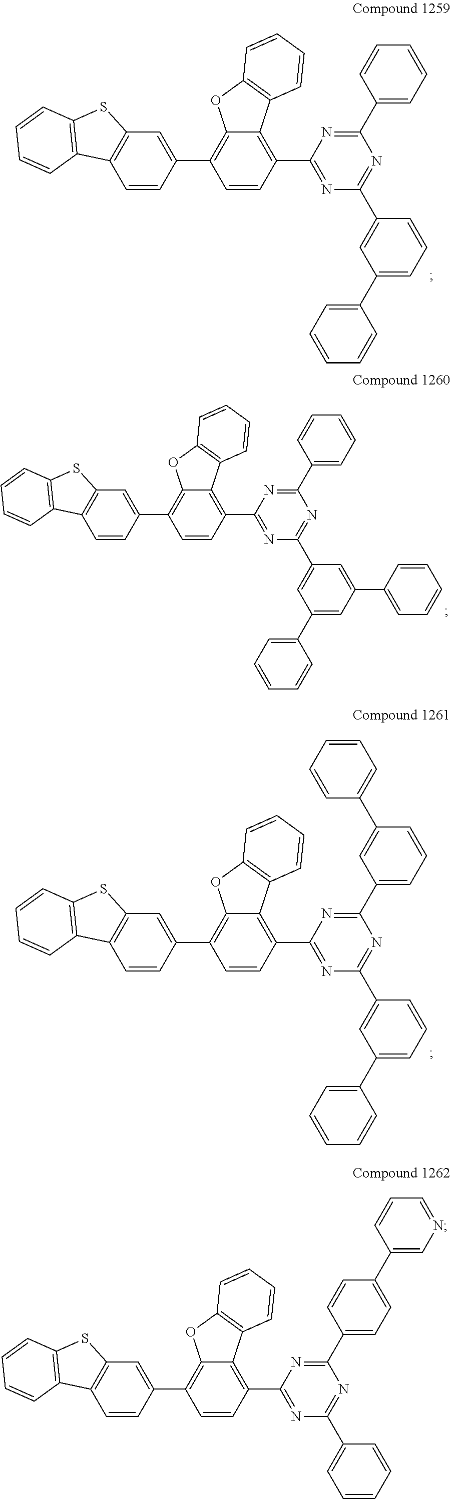 [SMILES: c1ccc(-c2cccc(-c3nc(-c4ccccc4)nc(-c4ccc(-c5ccc6c(c5)sc5ccccc56)c5oc6ccccc6c45)n3)c2)cc1 ; c1ccc(-c2cc(-c3ccccc3)cc(-c3nc(-c4ccccc4)nc(-c4ccc(-c5ccc6c(c5)sc5ccccc56)c5oc6ccccc6c45)n3)c2)cc1 ; c1ccc(-c2cccc(-c3nc(-c4cccc(-c5ccccc5)c4)nc(-c4ccc(-c5ccc6c(c5)sc5ccccc56)c5oc6ccccc6c45)n3)c2)cc1 ; c1ccc(-c2nc(-c3ccc(-c4cccnc4)cc3)nc(-c3ccc(-c4ccc5c(c4)sc4ccccc45)c4oc5ccccc5c34)n2)cc1]

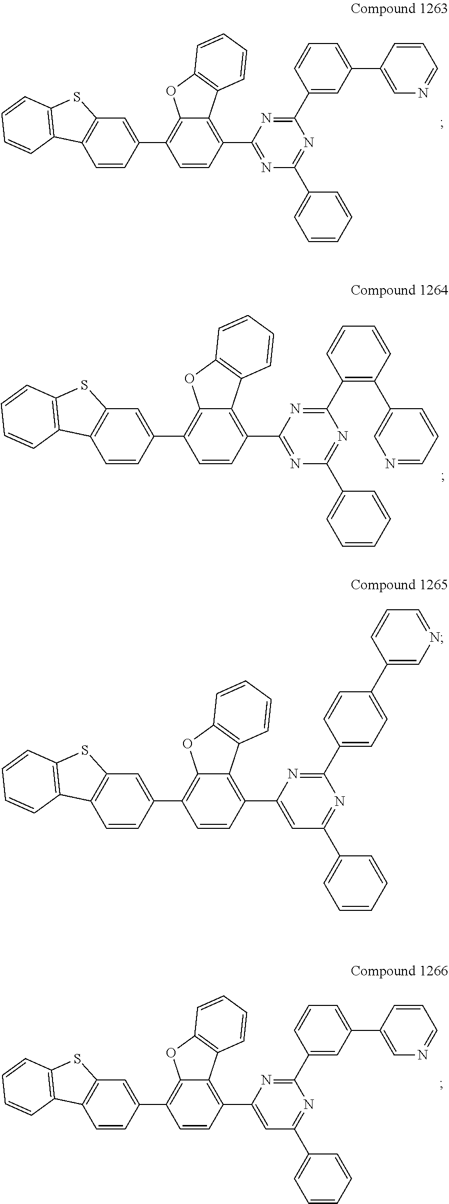 [SMILES: c1ccc(-c2nc(-c3cccc(-c4cccnc4)c3)nc(-c3ccc(-c4ccc5c(c4)sc4ccccc45)c4oc5ccccc5c34)n2)cc1 ; c1ccc(-c2nc(-c3ccccc3-c3cccnc3)nc(-c3ccc(-c4ccc5c(c4)sc4ccccc45)c4oc5ccccc5c34)n2)cc1 ; c1ccc(-c2cc(-c3ccc(-c4ccc5c(c4)sc4ccccc45)c4oc5ccccc5c34)nc(-c3ccc(-c4cccnc4)cc3)n2)cc1 ; c1ccc(-c2cc(-c3ccc(-c4ccc5c(c4)sc4ccccc45)c4oc5ccccc5c34)nc(-c3cccc(-c4cccnc4)c3)n2)cc1]

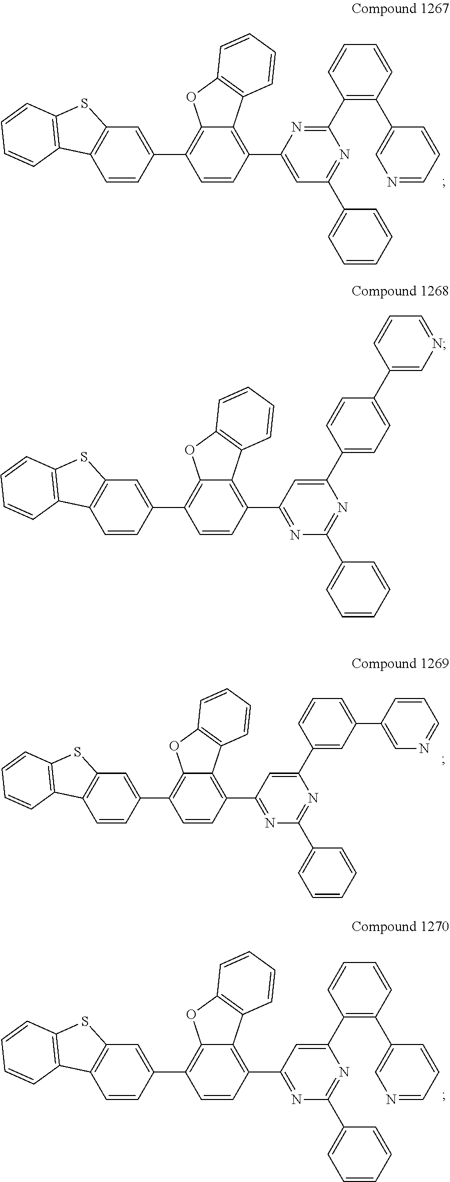 [SMILES: c1ccc(-c2cc(-c3ccc(-c4ccc5c(c4)sc4ccccc45)c4oc5ccccc5c34)nc(-c3ccccc3-c3cccnc3)n2)cc1 ; c1ccc(-c2nc(-c3ccc(-c4cccnc4)cc3)cc(-c3ccc(-c4ccc5c(c4)sc4ccccc45)c4oc5ccccc5c34)n2)cc1 ; c1ccc(-c2nc(-c3cccc(-c4cccnc4)c3)cc(-c3ccc(-c4ccc5c(c4)sc4ccccc45)c4oc5ccccc5c34)n2)cc1 ; c1ccc(-c2nc(-c3ccccc3-c3cccnc3)cc(-c3ccc(-c4ccc5c(c4)sc4ccccc45)c4oc5ccccc5c34)n2)cc1]

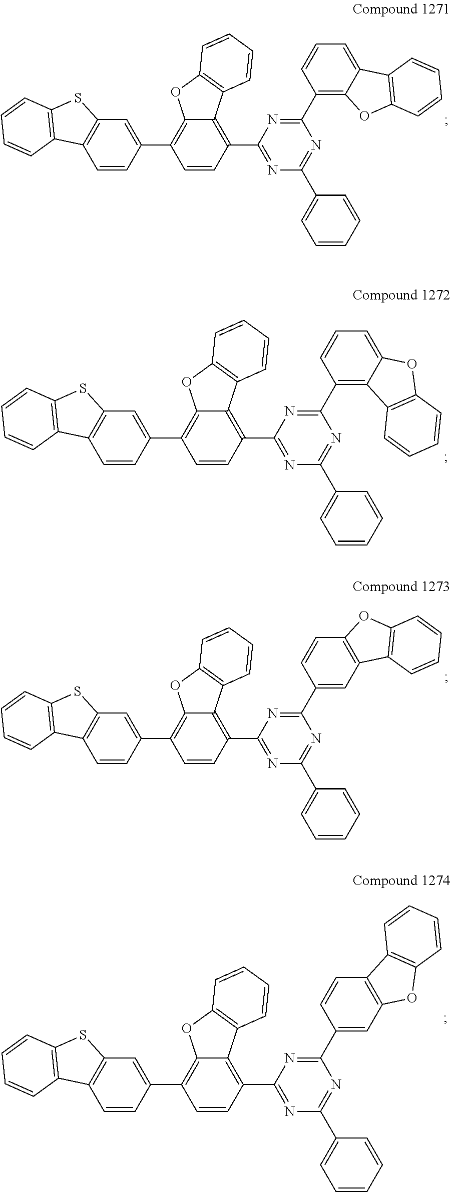 [SMILES: c1ccc(-c2nc(-c3cccc4c3oc3ccccc34)nc(-c3ccc(-c4ccc5c(c4)sc4ccccc45)c4oc5ccccc5c34)n2)cc1 ; c1ccc(-c2nc(-c3cccc4oc5ccccc5c34)nc(-c3ccc(-c4ccc5c(c4)sc4ccccc45)c4oc5ccccc5c34)n2)cc1 ; c1ccc(-c2nc(-c3ccc4oc5ccccc5c4c3)nc(-c3ccc(-c4ccc5c(c4)sc4ccccc45)c4oc5ccccc5c34)n2)cc1 ; c1ccc(-c2nc(-c3ccc4c(c3)oc3ccccc34)nc(-c3ccc(-c4ccc5c(c4)sc4ccccc45)c4oc5ccccc5c34)n2)cc1]

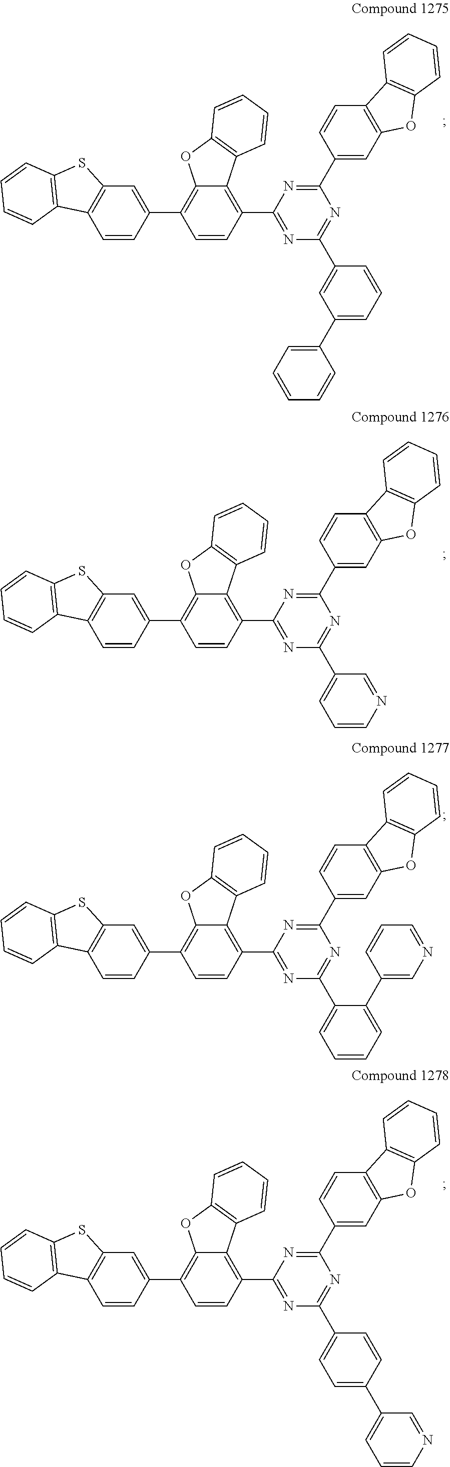 [SMILES: c1ccc(-c2cccc(-c3nc(-c4ccc5c(c4)oc4ccccc45)nc(-c4ccc(-c5ccc6c(c5)sc5ccccc56)c5oc6ccccc6c45)n3)c2)cc1 ; c1cncc(-c2nc(-c3ccc4c(c3)oc3ccccc34)nc(-c3ccc(-c4ccc5c(c4)sc4ccccc45)c4oc5ccccc5c34)n2)c1 ; c1cncc(-c2ccccc2-c2nc(-c3ccc4c(c3)oc3ccccc34)nc(-c3ccc(-c4ccc5c(c4)sc4ccccc45)c4oc5ccccc5c34)n2)c1 ; c1cncc(-c2ccc(-c3nc(-c4ccc5c(c4)oc4ccccc45)nc(-c4ccc(-c5ccc6c(c5)sc5ccccc56)c5oc6ccccc6c45)n3)cc2)c1]

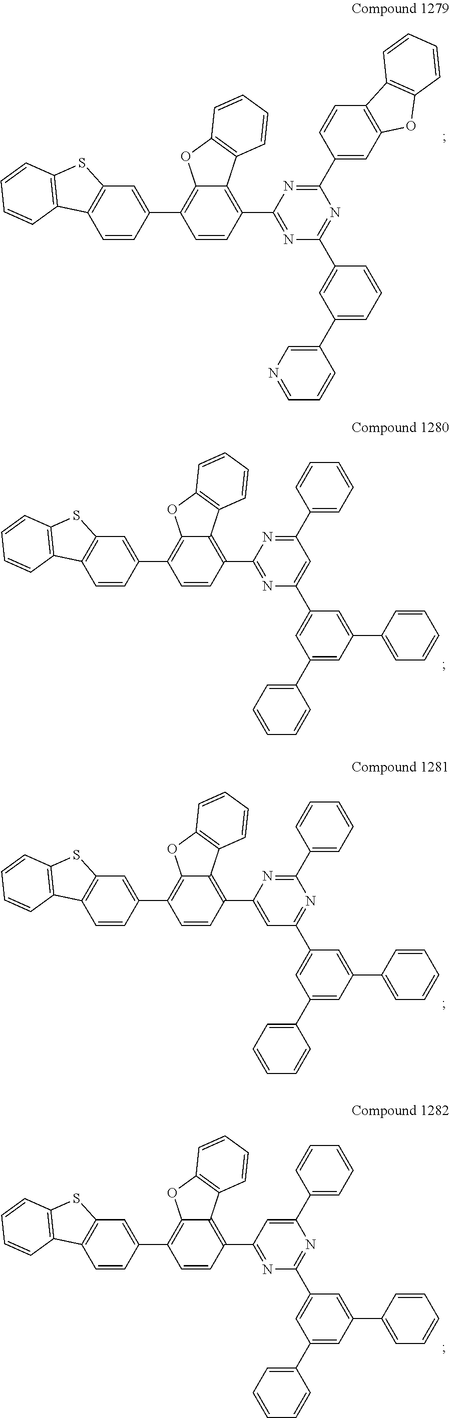 [SMILES: c1cncc(-c2cccc(-c3nc(-c4ccc5c(c4)oc4ccccc45)nc(-c4ccc(-c5ccc6c(c5)sc5ccccc56)c5oc6ccccc6c45)n3)c2)c1 ; c1ccc(-c2cc(-c3ccccc3)cc(-c3cc(-c4ccccc4)nc(-c4ccc(-c5ccc6c(c5)sc5ccccc56)c5oc6ccccc6c45)n3)c2)cc1 ; c1ccc(-c2cc(-c3ccccc3)cc(-c3cc(-c4ccc(-c5ccc6c(c5)sc5ccccc56)c5oc6ccccc6c45)nc(-c4ccccc4)n3)c2)cc1 ; c1ccc(-c2cc(-c3ccccc3)cc(-c3nc(-c4ccccc4)cc(-c4ccc(-c5ccc6c(c5)sc5ccccc56)c5oc6ccccc6c45)n3)c2)cc1]

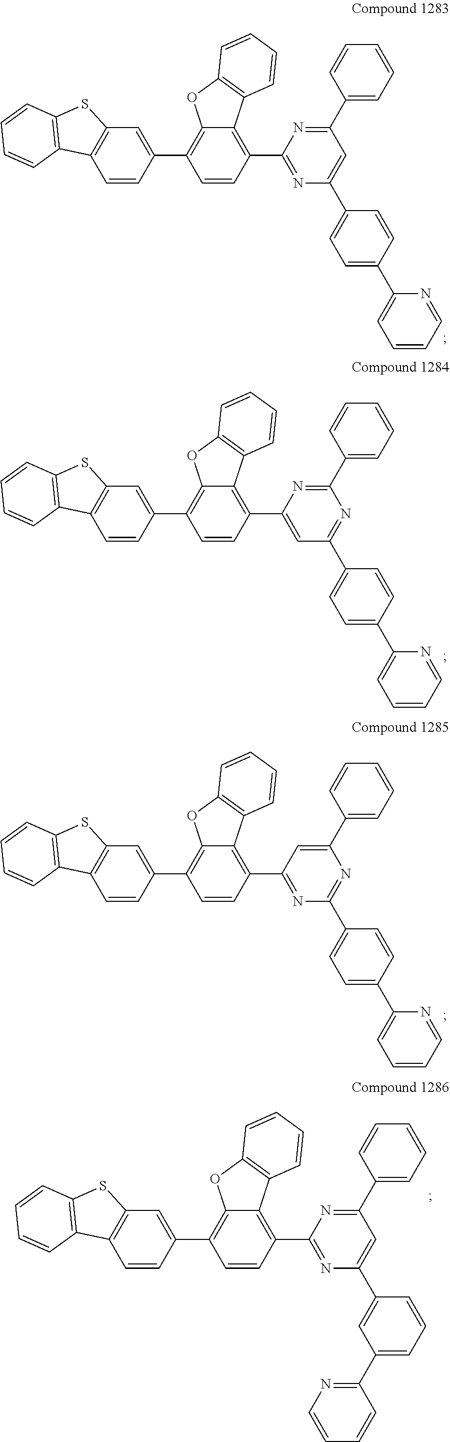 [SMILES: c1ccc(-c2cc(-c3ccc(-c4ccccn4)cc3)nc(-c3ccc(-c4ccc5c(c4)sc4ccccc45)c4oc5ccccc5c34)n2)cc1 ; c1ccc(-c2nc(-c3ccc(-c4ccccn4)cc3)cc(-c3ccc(-c4ccc5c(c4)sc4ccccc45)c4oc5ccccc5c34)n2)cc1 ; c1ccc(-c2cc(-c3ccc(-c4ccc5c(c4)sc4ccccc45)c4oc5ccccc5c34)nc(-c3ccc(-c4ccccn4)cc3)n2)cc1 ; c1ccc(-c2cc(-c3cccc(-c4ccccn4)c3)nc(-c3ccc(-c4ccc5c(c4)sc4ccccc45)c4oc5ccccc5c34)n2)cc1]

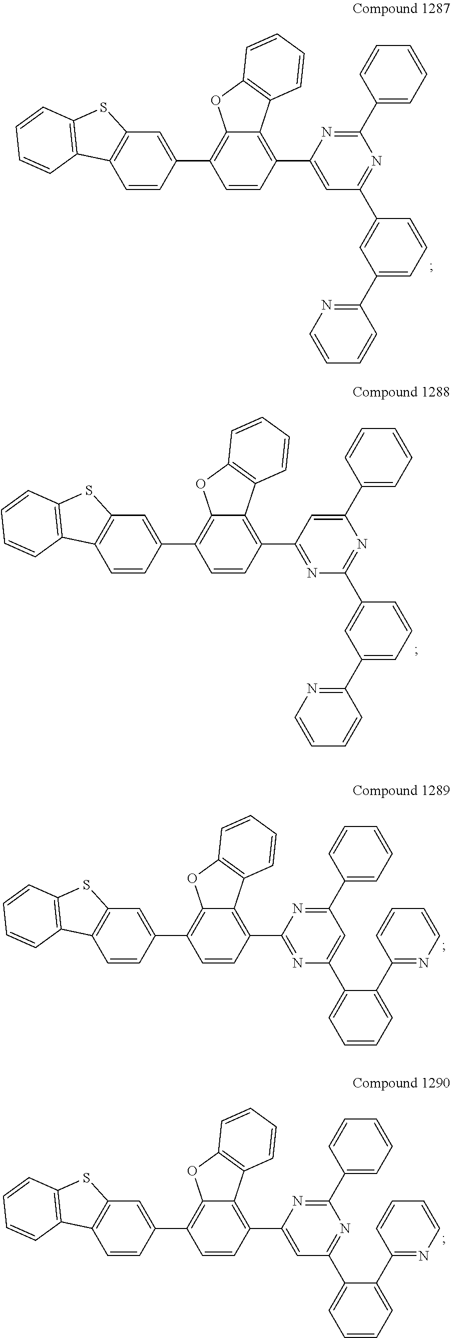 [SMILES: c1ccc(-c2nc(-c3cccc(-c4ccccn4)c3)cc(-c3ccc(-c4ccc5c(c4)sc4ccccc45)c4oc5ccccc5c34)n2)cc1 ; c1ccc(-c2cc(-c3ccc(-c4ccc5c(c4)sc4ccccc45)c4oc5ccccc5c34)nc(-c3cccc(-c4ccccn4)c3)n2)cc1 ; c1ccc(-c2cc(-c3ccccc3-c3ccccn3)nc(-c3ccc(-c4ccc5c(c4)sc4ccccc45)c4oc5ccccc5c34)n2)cc1 ; c1ccc(-c2nc(-c3ccccc3-c3ccccn3)cc(-c3ccc(-c4ccc5c(c4)sc4ccccc45)c4oc5ccccc5c34)n2)cc1]

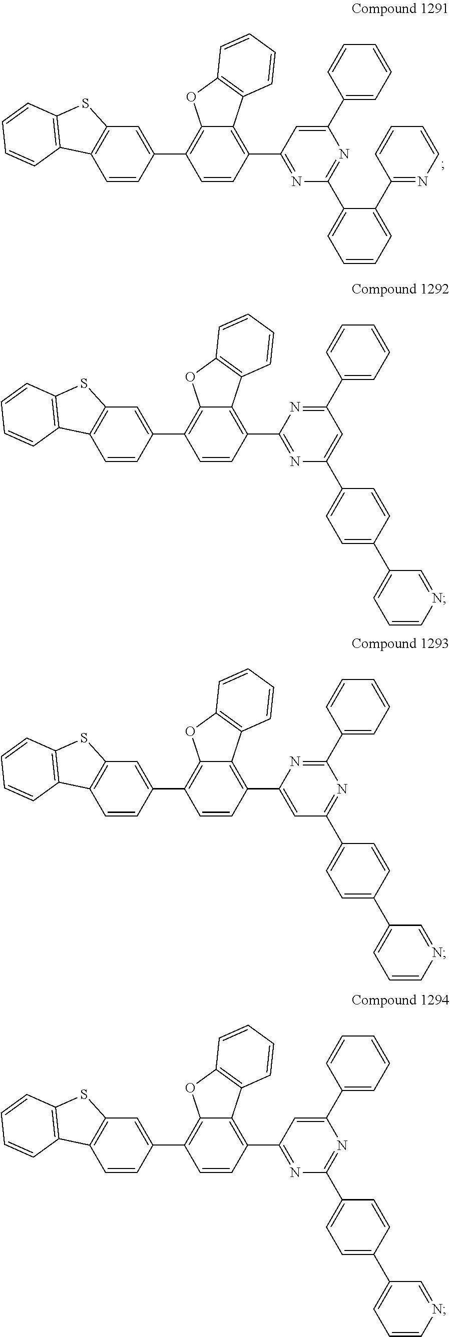 [SMILES: c1ccc(-c2cc(-c3ccc(-c4ccc5c(c4)sc4ccccc45)c4oc5ccccc5c34)nc(-c3ccccc3-c3ccccn3)n2)cc1 ; c1ccc(-c2cc(-c3ccc(-c4cccnc4)cc3)nc(-c3ccc(-c4ccc5c(c4)sc4ccccc45)c4oc5ccccc5c34)n2)cc1 ; c1ccc(-c2nc(-c3ccc(-c4cccnc4)cc3)cc(-c3ccc(-c4ccc5c(c4)sc4ccccc45)c4oc5ccccc5c34)n2)cc1 ; c1ccc(-c2cc(-c3ccc(-c4ccc5c(c4)sc4ccccc45)c4oc5ccccc5c34)nc(-c3ccc(-c4cccnc4)cc3)n2)cc1]

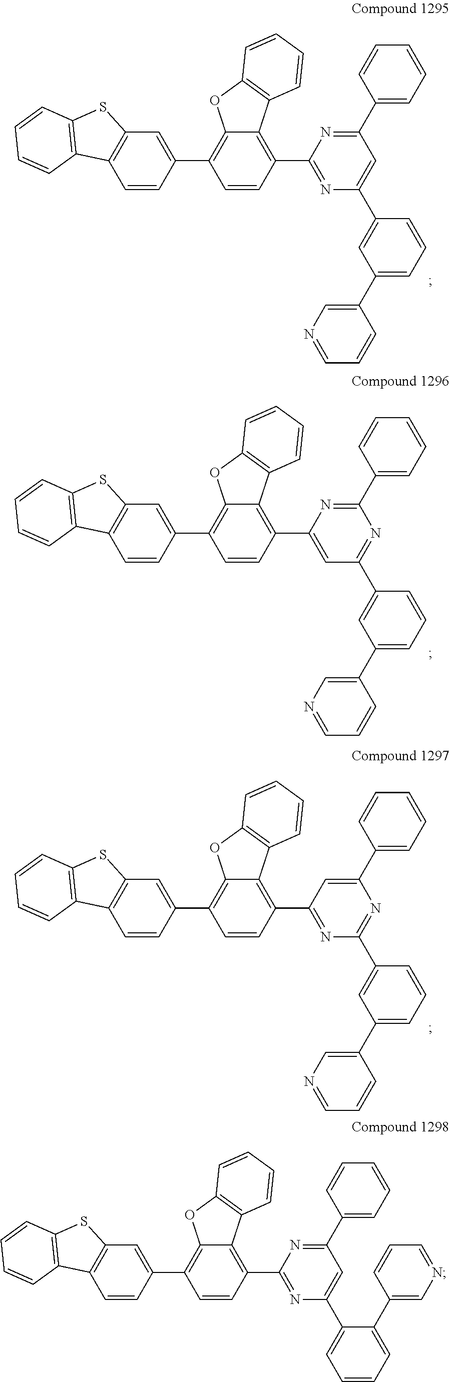 [SMILES: c1ccc(-c2cc(-c3cccc(-c4cccnc4)c3)nc(-c3ccc(-c4ccc5c(c4)sc4ccccc45)c4oc5ccccc5c34)n2)cc1 ; c1ccc(-c2nc(-c3cccc(-c4cccnc4)c3)cc(-c3ccc(-c4ccc5c(c4)sc4ccccc45)c4oc5ccccc5c34)n2)cc1 ; c1ccc(-c2cc(-c3ccc(-c4ccc5c(c4)sc4ccccc45)c4oc5ccccc5c34)nc(-c3cccc(-c4cccnc4)c3)n2)cc1 ; c1ccc(-c2cc(-c3ccccc3-c3cccnc3)nc(-c3ccc(-c4ccc5c(c4)sc4ccccc45)c4oc5ccccc5c34)n2)cc1]

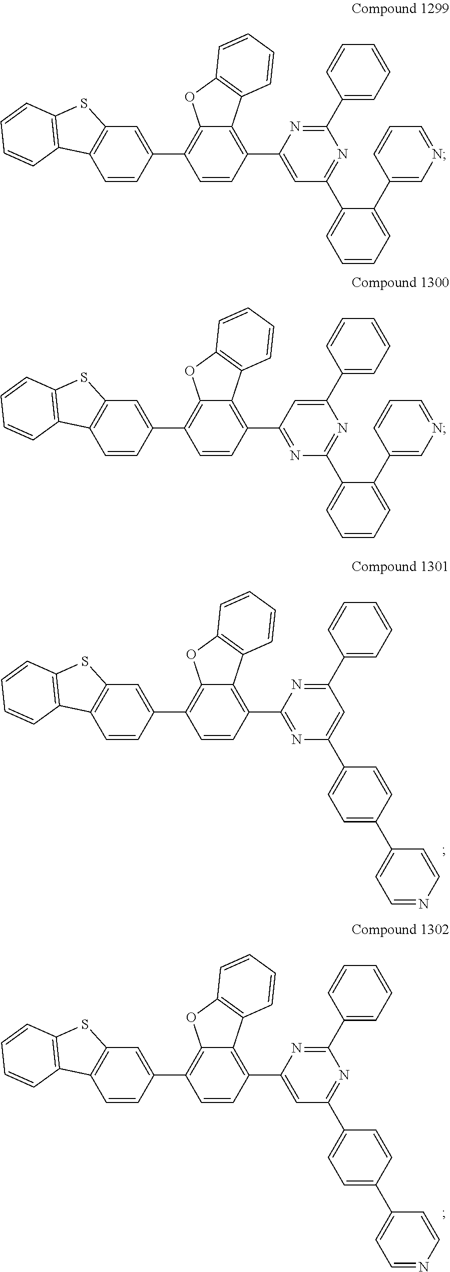 [SMILES: c1ccc(-c2nc(-c3ccccc3-c3cccnc3)cc(-c3ccc(-c4ccc5c(c4)sc4ccccc45)c4oc5ccccc5c34)n2)cc1 ; c1ccc(-c2cc(-c3ccc(-c4ccc5c(c4)sc4ccccc45)c4oc5ccccc5c34)nc(-c3ccccc3-c3cccnc3)n2)cc1 ; c1ccc(-c2cc(-c3ccc(-c4ccncc4)cc3)nc(-c3ccc(-c4ccc5c(c4)sc4ccccc45)c4oc5ccccc5c34)n2)cc1 ; c1ccc(-c2nc(-c3ccc(-c4ccncc4)cc3)cc(-c3ccc(-c4ccc5c(c4)sc4ccccc45)c4oc5ccccc5c34)n2)cc1]

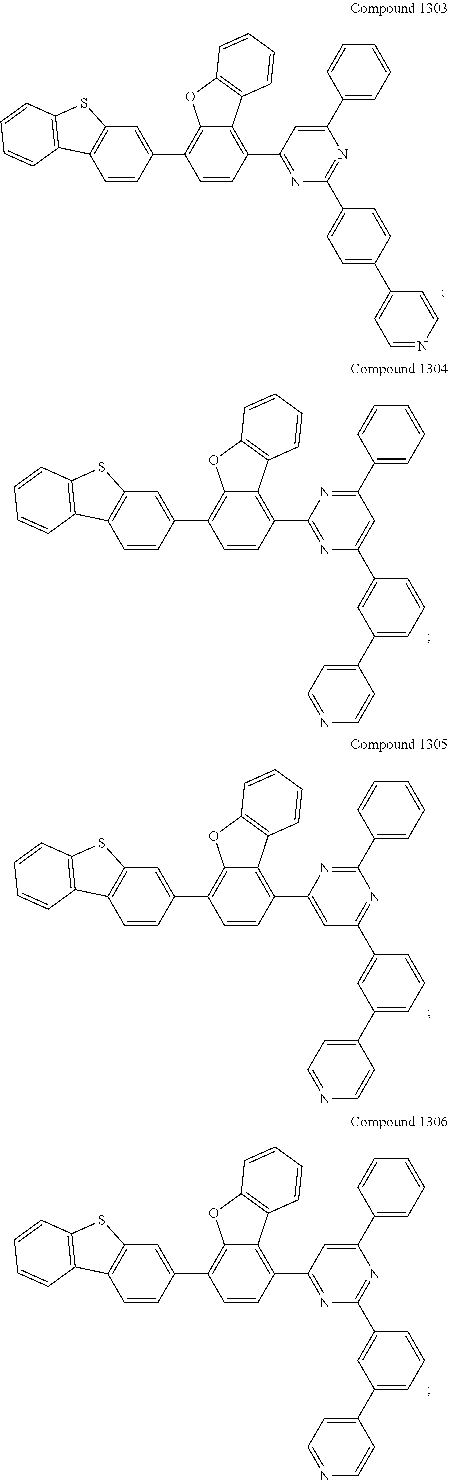 [SMILES: c1ccc(-c2cc(-c3ccc(-c4ccc5c(c4)sc4ccccc45)c4oc5ccccc5c34)nc(-c3ccc(-c4ccncc4)cc3)n2)cc1 ; c1ccc(-c2cc(-c3cccc(-c4ccncc4)c3)nc(-c3ccc(-c4ccc5c(c4)sc4ccccc45)c4oc5ccccc5c34)n2)cc1 ; c1ccc(-c2nc(-c3cccc(-c4ccncc4)c3)cc(-c3ccc(-c4ccc5c(c4)sc4ccccc45)c4oc5ccccc5c34)n2)cc1 ; c1ccc(-c2cc(-c3ccc(-c4ccc5c(c4)sc4ccccc45)c4oc5ccccc5c34)nc(-c3cccc(-c4ccncc4)c3)n2)cc1]

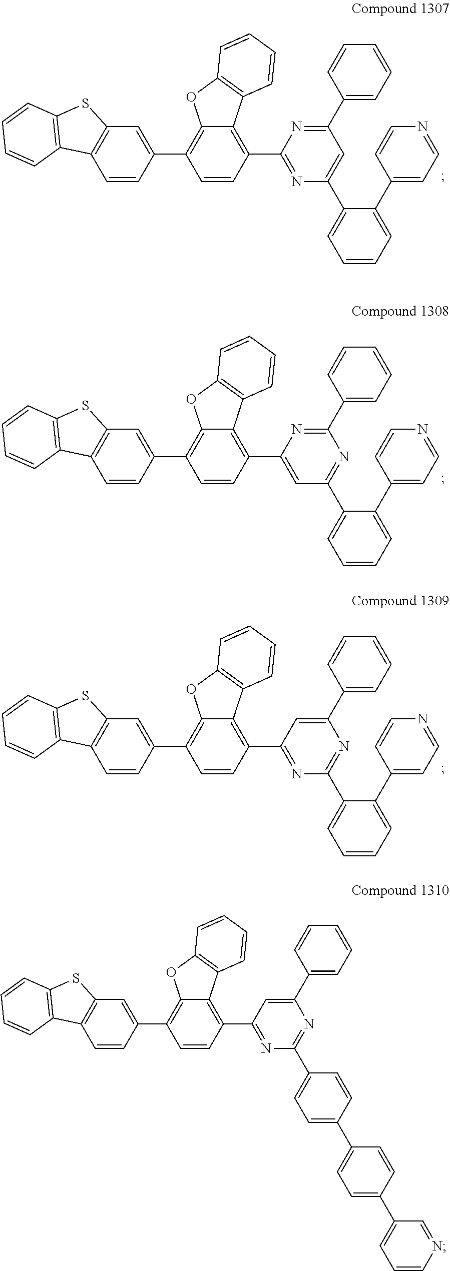 [SMILES: c1ccc(-c2cc(-c3ccccc3-c3ccncc3)nc(-c3ccc(-c4ccc5c(c4)sc4ccccc45)c4oc5ccccc5c34)n2)cc1 ; c1ccc(-c2nc(-c3ccccc3-c3ccncc3)cc(-c3ccc(-c4ccc5c(c4)sc4ccccc45)c4oc5ccccc5c34)n2)cc1 ; c1ccc(-c2cc(-c3ccc(-c4ccc5c(c4)sc4ccccc45)c4oc5ccccc5c34)nc(-c3ccccc3-c3ccncc3)n2)cc1 ; c1ccc(-c2cc(-c3ccc(-c4ccc5c(c4)sc4ccccc45)c4oc5ccccc5c34)nc(-c3ccc(-c4ccc(-c5cccnc5)cc4)cc3)n2)cc1]

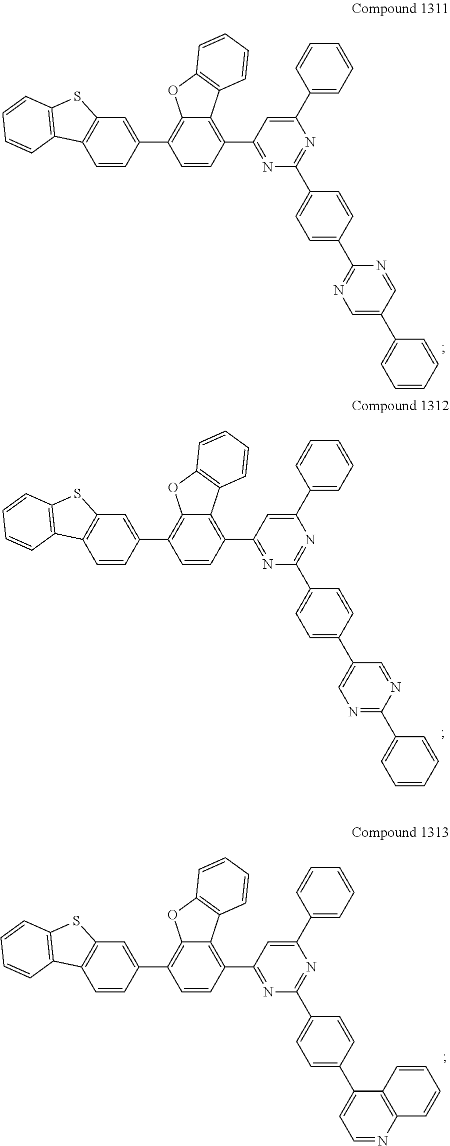 [SMILES: c1ccc(-c2cnc(-c3ccc(-c4nc(-c5ccccc5)cc(-c5ccc(-c6ccc7c(c6)sc6ccccc67)c6oc7ccccc7c56)n4)cc3)nc2)cc1 ; c1ccc(-c2cc(-c3ccc(-c4ccc5c(c4)sc4ccccc45)c4oc5ccccc5c34)nc(-c3ccc(-c4cnc(-c5ccccc5)nc4)cc3)n2)cc1 ; c1ccc(-c2cc(-c3ccc(-c4ccc5c(c4)sc4ccccc45)c4oc5ccccc5c34)nc(-c3ccc(-c4ccnc5ccccc45)cc3)n2)cc1]

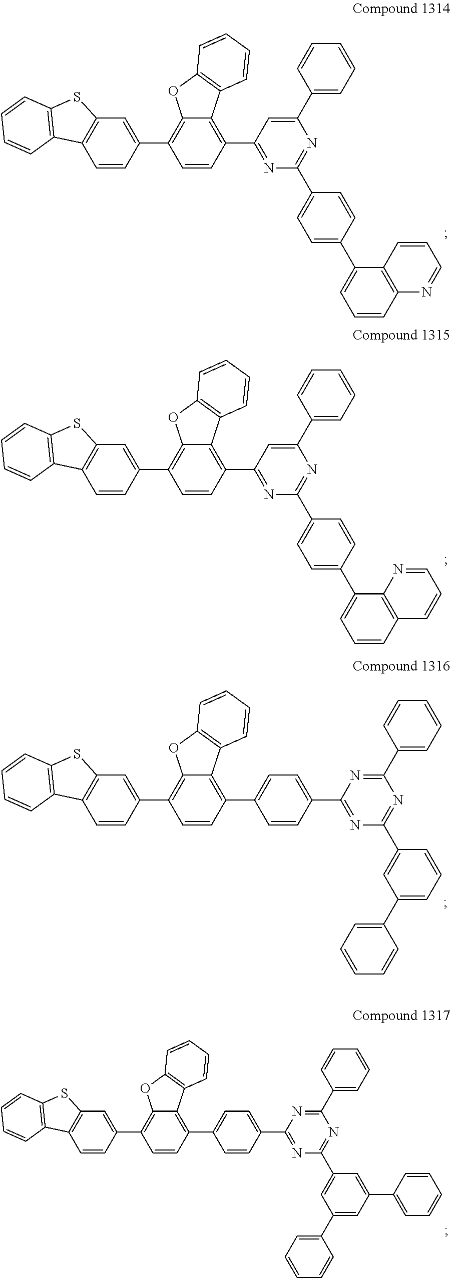 [SMILES: c1ccc(-c2cc(-c3ccc(-c4ccc5c(c4)sc4ccccc45)c4oc5ccccc5c34)nc(-c3ccc(-c4cccc5ncccc45)cc3)n2)cc1 ; c1ccc(-c2cc(-c3ccc(-c4ccc5c(c4)sc4ccccc45)c4oc5ccccc5c34)nc(-c3ccc(-c4cccc5cccnc45)cc3)n2)cc1 ; c1ccc(-c2cccc(-c3nc(-c4ccccc4)nc(-c4ccc(-c5ccc(-c6ccc7c(c6)sc6ccccc67)c6oc7ccccc7c56)cc4)n3)c2)cc1 ; c1ccc(-c2cc(-c3ccccc3)cc(-c3nc(-c4ccccc4)nc(-c4ccc(-c5ccc(-c6ccc7c(c6)sc6ccccc67)c6oc7ccccc7c56)cc4)n3)c2)cc1]

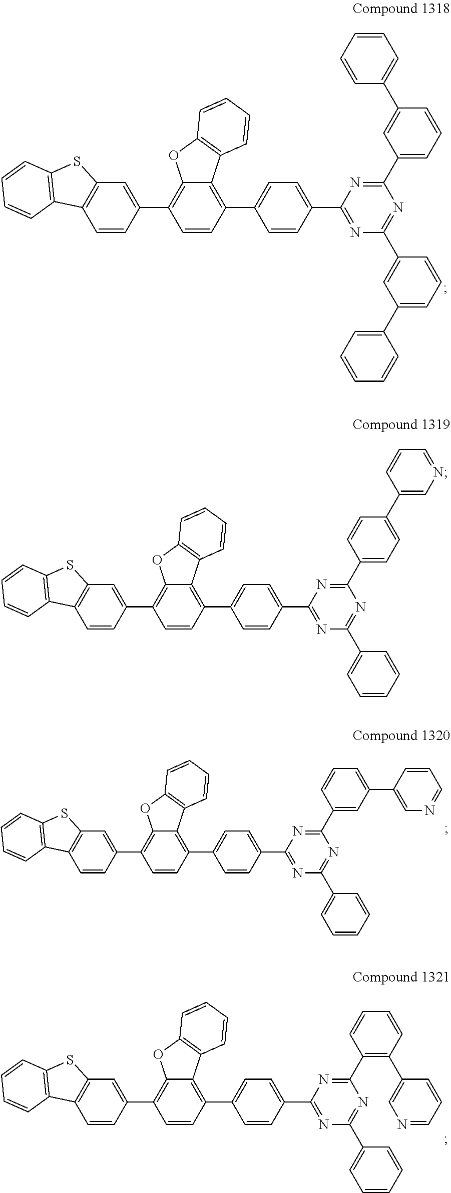 [SMILES: c1ccc(-c2cccc(-c3nc(-c4ccc(-c5ccc(-c6ccc7c(c6)sc6ccccc67)c6oc7ccccc7c56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5ccc6c(c5)sc5ccccc56)c5oc6ccccc6c45)cc3)n2)cc1 ; c1ccc(-c2nc(-c3ccc(-c4ccc(-c5ccc6c(c5)sc5ccccc56)c5oc6ccccc6c45)cc3)nc(-c3cccc(-c4cccnc4)c3)n2)cc1 ; c1ccc(-c2nc(-c3ccc(-c4ccc(-c5ccc6c(c5)sc5ccccc56)c5oc6ccccc6c45)cc3)nc(-c3ccccc3-c3cccnc3)n2)cc1]

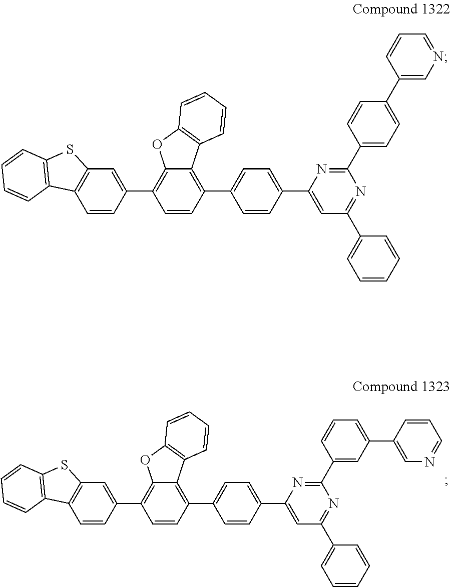 [SMILES: c1ccc(-c2cc(-c3ccc(-c4ccc(-c5ccc6c(c5)sc5ccccc56)c5oc6ccccc6c45)cc3)nc(-c3ccc(-c4cccnc4)cc3)n2)cc1 ; c1ccc(-c2cc(-c3ccc(-c4ccc(-c5ccc6c(c5)sc5ccccc56)c5oc6ccccc6c45)cc3)nc(-c3cccc(-c4cccnc4)c3)n2)cc1]

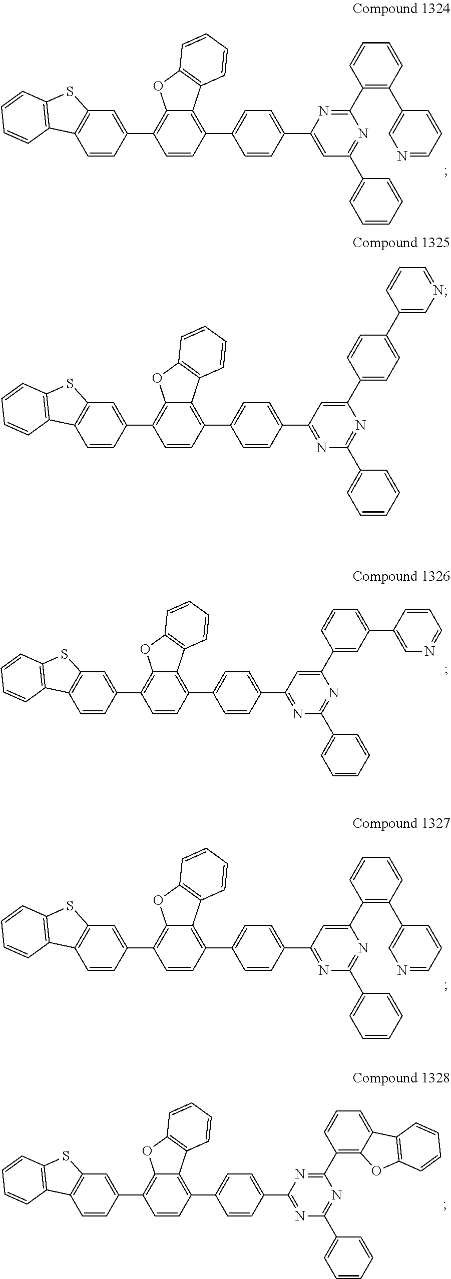 [SMILES: c1ccc(-c2cc(-c3ccc(-c4ccc(-c5ccc6c(c5)sc5ccccc56)c5oc6ccccc6c45)cc3)nc(-c3ccccc3-c3cccnc3)n2)cc1 ; c1ccc(-c2nc(-c3ccc(-c4cccnc4)cc3)cc(-c3ccc(-c4ccc(-c5ccc6c(c5)sc5ccccc56)c5oc6ccccc6c45)cc3)n2)cc1 ; c1ccc(-c2nc(-c3ccc(-c4ccc(-c5ccc6c(c5)sc5ccccc56)c5oc6ccccc6c45)cc3)cc(-c3cccc(-c4cccnc4)c3)n2)cc1 ; c1ccc(-c2nc(-c3ccc(-c4ccc(-c5ccc6c(c5)sc5ccccc56)c5oc6ccccc6c45)cc3)cc(-c3ccccc3-c3cccnc3)n2)cc1 ; c1ccc(-c2nc(-c3ccc(-c4ccc(-c5ccc6c(c5)sc5ccccc56)c5oc6ccccc6c45)cc3)nc(-c3cccc4c3oc3ccccc34)n2)cc1]

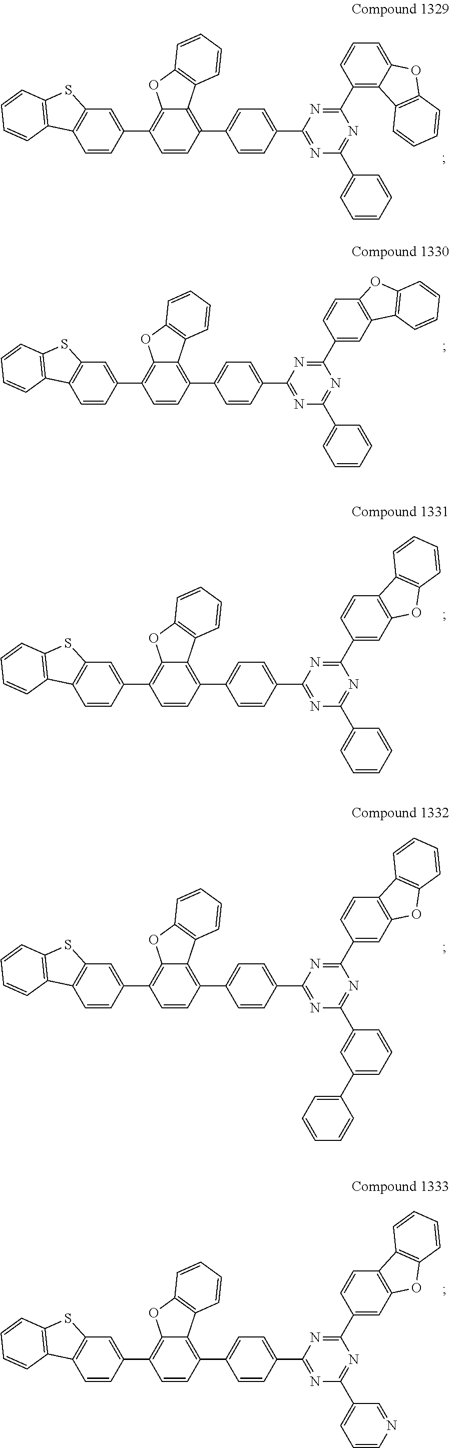

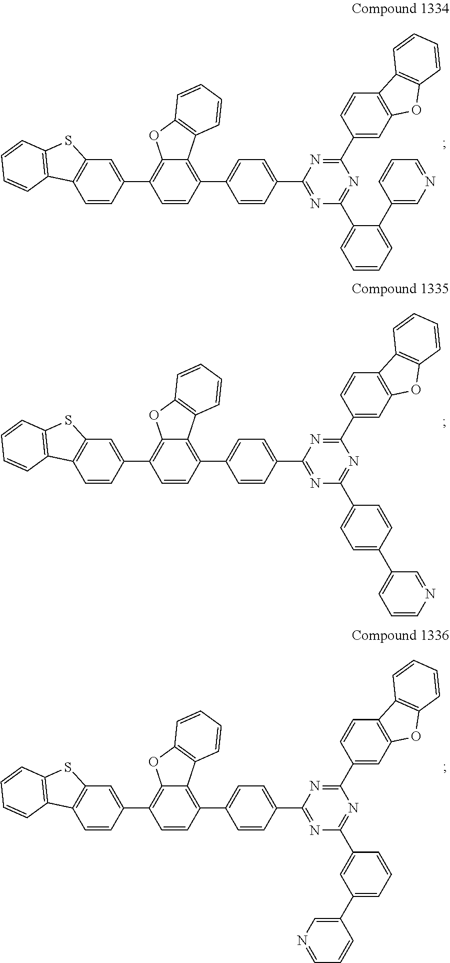 [SMILES: c1cncc(-c2ccccc2-c2nc(-c3ccc(-c4ccc(-c5ccc6c(c5)sc5ccccc56)c5oc6ccccc6c45)cc3)nc(-c3ccc4c(c3)oc3ccccc34)n2)c1 ; c1cncc(-c2ccc(-c3nc(-c4ccc(-c5ccc(-c6ccc7c(c6)sc6ccccc67)c6oc7ccccc7c56)cc4)nc(-c4ccc5c(c4)oc4ccccc45)n3)cc2)c1 ; c1cncc(-c2cccc(-c3nc(-c4ccc(-c5ccc(-c6ccc7c(c6)sc6ccccc67)c6oc7ccccc7c56)cc4)nc(-c4ccc5c(c4)oc4ccccc45)n3)c2)c1]

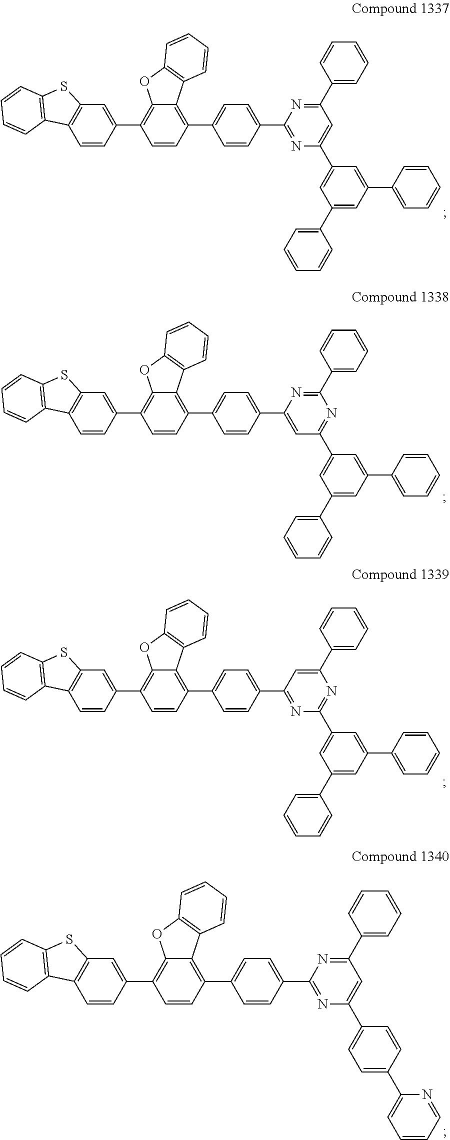 [SMILES: c1ccc(-c2cc(-c3ccccc3)cc(-c3cc(-c4ccccc4)nc(-c4ccc(-c5ccc(-c6ccc7c(c6)sc6ccccc67)c6oc7ccccc7c56)cc4)n3)c2)cc1 ; c1ccc(-c2cc(-c3ccccc3)cc(-c3cc(-c4ccc(-c5ccc(-c6ccc7c(c6)sc6ccccc67)c6oc7ccccc7c56)cc4)nc(-c4ccccc4)n3)c2)cc1 ; c1ccc(-c2cc(-c3ccccc3)cc(-c3nc(-c4ccccc4)cc(-c4ccc(-c5ccc(-c6ccc7c(c6)sc6ccccc67)c6oc7ccccc7c56)cc4)n3)c2)cc1 ; c1ccc(-c2cc(-c3ccc(-c4ccccn4)cc3)nc(-c3ccc(-c4ccc(-c5ccc6c(c5)sc5ccccc56)c5oc6ccccc6c45)cc3)n2)cc1]

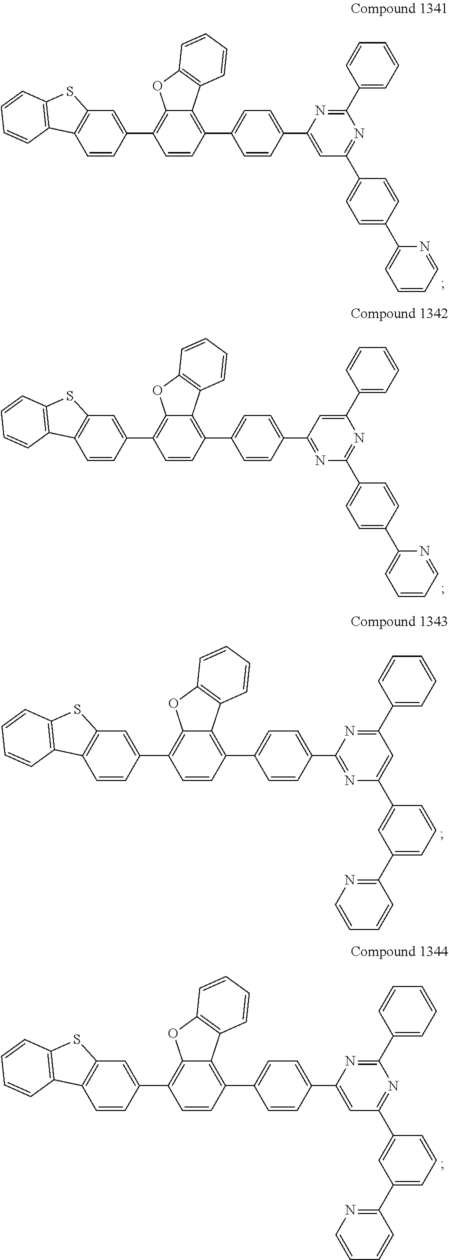 [SMILES: c1ccc(-c2nc(-c3ccc(-c4ccccn4)cc3)cc(-c3ccc(-c4ccc(-c5ccc6c(c5)sc5ccccc56)c5oc6ccccc6c45)cc3)n2)cc1 ; c1ccc(-c2cc(-c3ccc(-c4ccc(-c5ccc6c(c5)sc5ccccc56)c5oc6ccccc6c45)cc3)nc(-c3ccc(-c4ccccn4)cc3)n2)cc1 ; c1ccc(-c2cc(-c3cccc(-c4ccccn4)c3)nc(-c3ccc(-c4ccc(-c5ccc6c(c5)sc5ccccc56)c5oc6ccccc6c45)cc3)n2)cc1 ; c1ccc(-c2nc(-c3ccc(-c4ccc(-c5ccc6c(c5)sc5ccccc56)c5oc6ccccc6c45)cc3)cc(-c3cccc(-c4ccccn4)c3)n2)cc1]

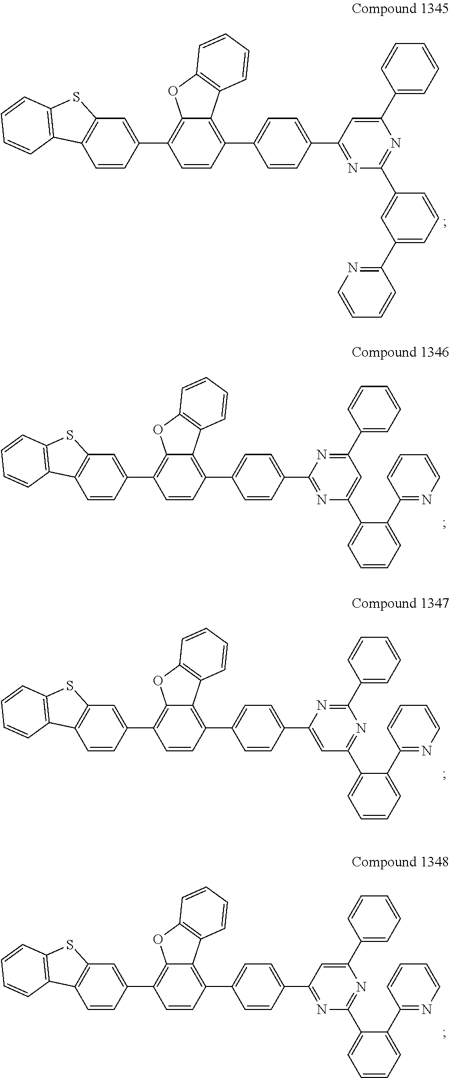 [SMILES: c1ccc(-c2cc(-c3ccc(-c4ccc(-c5ccc6c(c5)sc5ccccc56)c5oc6ccccc6c45)cc3)nc(-c3cccc(-c4ccccn4)c3)n2)cc1 ; c1ccc(-c2cc(-c3ccccc3-c3ccccn3)nc(-c3ccc(-c4ccc(-c5ccc6c(c5)sc5ccccc56)c5oc6ccccc6c45)cc3)n2)cc1 ; c1ccc(-c2nc(-c3ccc(-c4ccc(-c5ccc6c(c5)sc5ccccc56)c5oc6ccccc6c45)cc3)cc(-c3ccccc3-c3ccccn3)n2)cc1 ; c1ccc(-c2cc(-c3ccc(-c4ccc(-c5ccc6c(c5)sc5ccccc56)c5oc6ccccc6c45)cc3)nc(-c3ccccc3-c3ccccn3)n2)cc1]

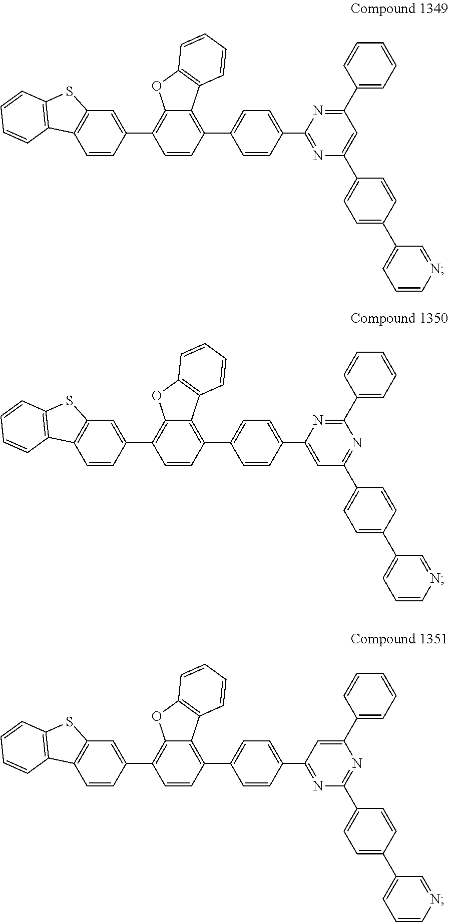 [SMILES: c1ccc(-c2cc(-c3ccc(-c4cccnc4)cc3)nc(-c3ccc(-c4ccc(-c5ccc6c(c5)sc5ccccc56)c5oc6ccccc6c45)cc3)n2)cc1 ; c1ccc(-c2nc(-c3ccc(-c4cccnc4)cc3)cc(-c3ccc(-c4ccc(-c5ccc6c(c5)sc5ccccc56)c5oc6ccccc6c45)cc3)n2)cc1 ; c1ccc(-c2cc(-c3ccc(-c4ccc(-c5ccc6c(c5)sc5ccccc56)c5oc6ccccc6c45)cc3)nc(-c3ccc(-c4cccnc4)cc3)n2)cc1]

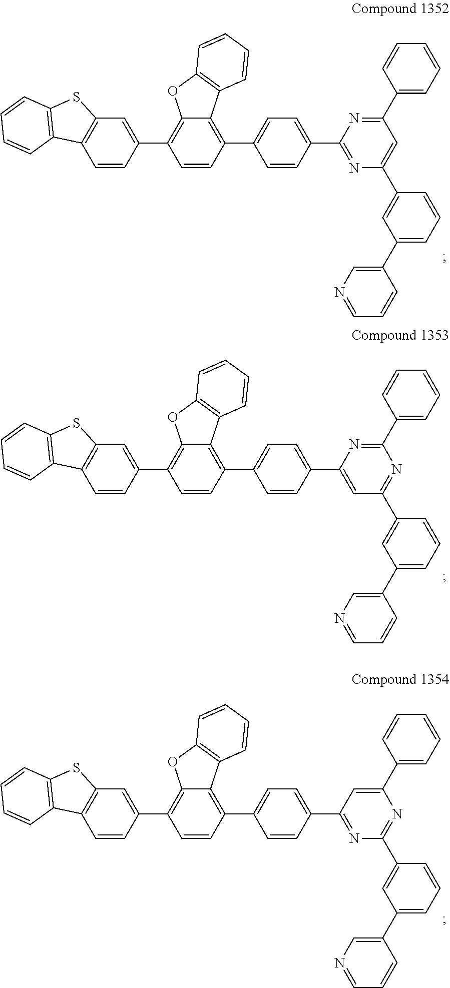 [SMILES: c1ccc(-c2cc(-c3cccc(-c4cccnc4)c3)nc(-c3ccc(-c4ccc(-c5ccc6c(c5)sc5ccccc56)c5oc6ccccc6c45)cc3)n2)cc1 ; c1ccc(-c2nc(-c3ccc(-c4ccc(-c5ccc6c(c5)sc5ccccc56)c5oc6ccccc6c45)cc3)cc(-c3cccc(-c4cccnc4)c3)n2)cc1 ; c1ccc(-c2cc(-c3ccc(-c4ccc(-c5ccc6c(c5)sc5ccccc56)c5oc6ccccc6c45)cc3)nc(-c3cccc(-c4cccnc4)c3)n2)cc1]

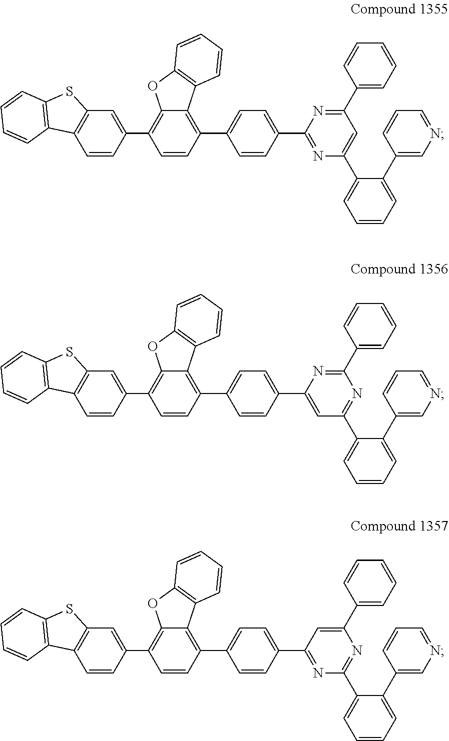 [SMILES: c1ccc(-c2cc(-c3ccccc3-c3cccnc3)nc(-c3ccc(-c4ccc(-c5ccc6c(c5)sc5ccccc56)c5oc6ccccc6c45)cc3)n2)cc1 ; c1ccc(-c2nc(-c3ccc(-c4ccc(-c5ccc6c(c5)sc5ccccc56)c5oc6ccccc6c45)cc3)cc(-c3ccccc3-c3cccnc3)n2)cc1 ; c1ccc(-c2cc(-c3ccc(-c4ccc(-c5ccc6c(c5)sc5ccccc56)c5oc6ccccc6c45)cc3)nc(-c3ccccc3-c3cccnc3)n2)cc1]

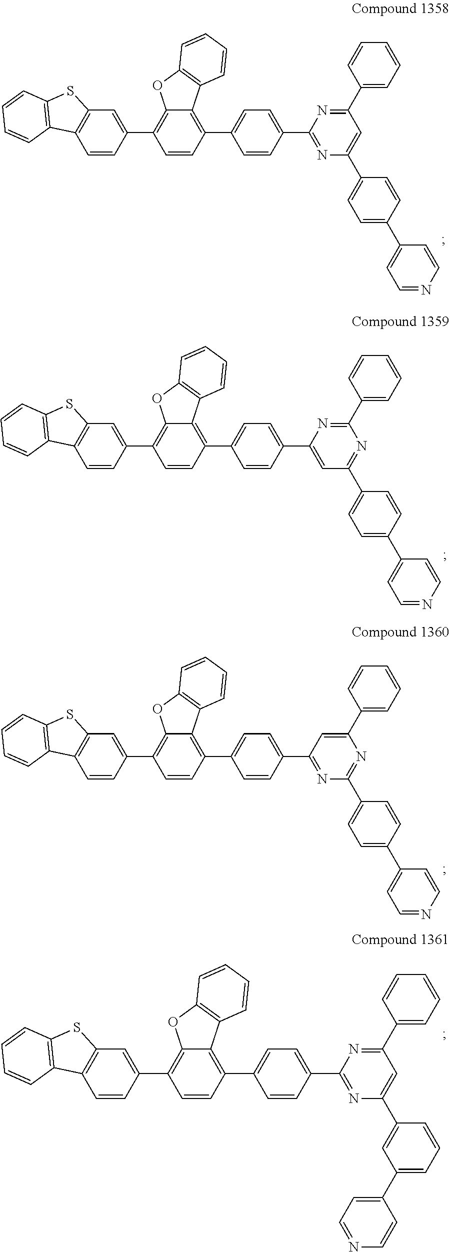 [SMILES: c1ccc(-c2cc(-c3ccc(-c4ccncc4)cc3)nc(-c3ccc(-c4ccc(-c5ccc6c(c5)sc5ccccc56)c5oc6ccccc6c45)cc3)n2)cc1 ; c1ccc(-c2nc(-c3ccc(-c4ccncc4)cc3)cc(-c3ccc(-c4ccc(-c5ccc6c(c5)sc5ccccc56)c5oc6ccccc6c45)cc3)n2)cc1 ; c1ccc(-c2cc(-c3ccc(-c4ccc(-c5ccc6c(c5)sc5ccccc56)c5oc6ccccc6c45)cc3)nc(-c3ccc(-c4ccncc4)cc3)n2)cc1 ; c1ccc(-c2cc(-c3cccc(-c4ccncc4)c3)nc(-c3ccc(-c4ccc(-c5ccc6c(c5)sc5ccccc56)c5oc6ccccc6c45)cc3)n2)cc1]

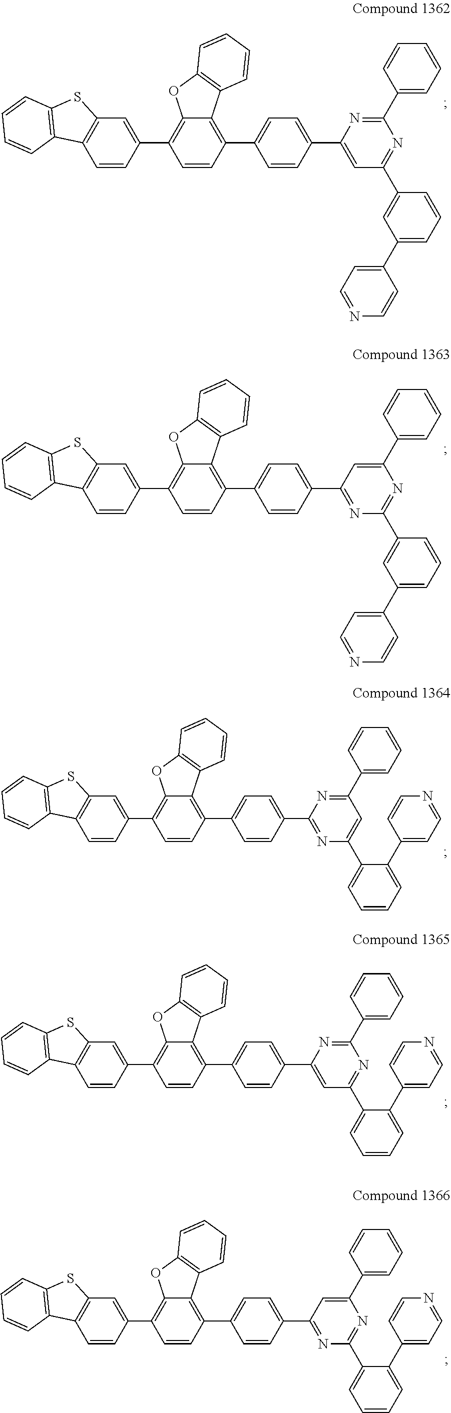 [SMILES: c1ccc(-c2nc(-c3ccc(-c4ccc(-c5ccc6c(c5)sc5ccccc56)c5oc6ccccc6c45)cc3)cc(-c3cccc(-c4ccncc4)c3)n2)cc1 ; c1ccc(-c2cc(-c3ccc(-c4ccc(-c5ccc6c(c5)sc5ccccc56)c5oc6ccccc6c45)cc3)nc(-c3cccc(-c4ccncc4)c3)n2)cc1 ; c1ccc(-c2cc(-c3ccccc3-c3ccncc3)nc(-c3ccc(-c4ccc(-c5ccc6c(c5)sc5ccccc56)c5oc6ccccc6c45)cc3)n2)cc1 ; c1ccc(-c2nc(-c3ccc(-c4ccc(-c5ccc6c(c5)sc5ccccc56)c5oc6ccccc6c45)cc3)cc(-c3ccccc3-c3ccncc3)n2)cc1 ; c1ccc(-c2cc(-c3ccc(-c4ccc(-c5ccc6c(c5)sc5ccccc56)c5oc6ccccc6c45)cc3)nc(-c3ccccc3-c3ccncc3)n2)cc1]

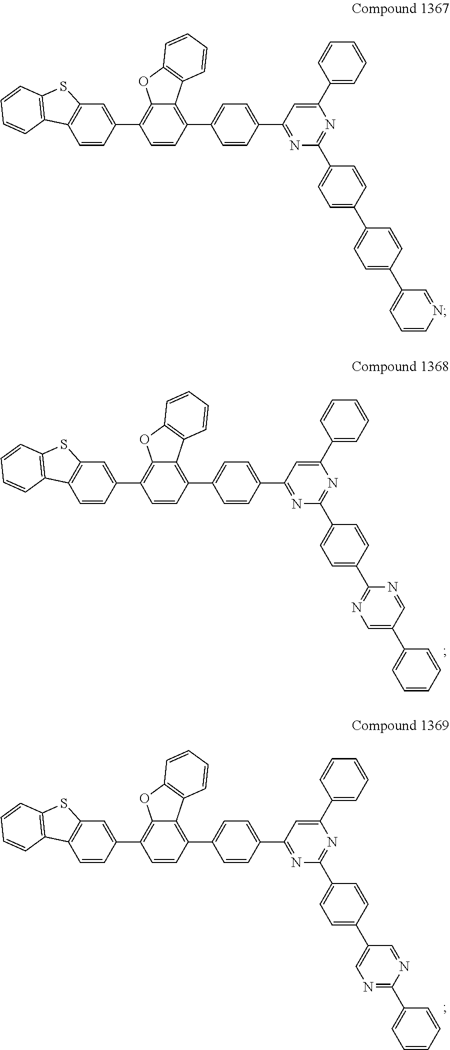 [SMILES: c1ccc(-c2cc(-c3ccc(-c4ccc(-c5ccc6c(c5)sc5ccccc56)c5oc6ccccc6c45)cc3)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc(-c7ccc8c(c7)sc7ccccc78)c7oc8ccccc8c67)cc5)n4)cc3)nc2)cc1 ; c1ccc(-c2cc(-c3ccc(-c4ccc(-c5ccc6c(c5)sc5ccccc56)c5oc6ccccc6c45)cc3)nc(-c3ccc(-c4cnc(-c5ccccc5)nc4)cc3)n2)cc1]

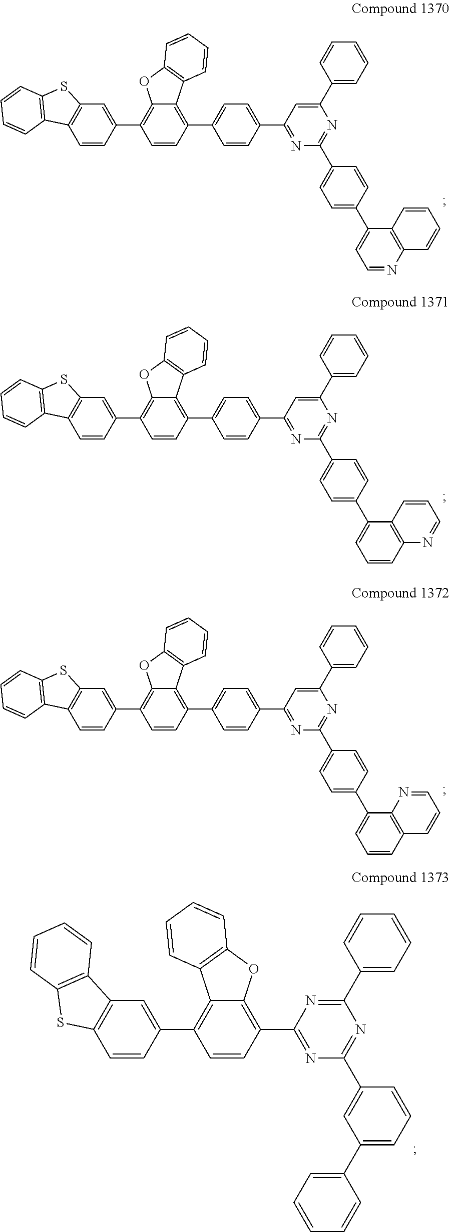 [SMILES: c1ccc(-c2cc(-c3ccc(-c4ccc(-c5ccc6c(c5)sc5ccccc56)c5oc6ccccc6c45)cc3)nc(-c3ccc(-c4ccnc5ccccc45)cc3)n2)cc1 ; c1ccc(-c2cc(-c3ccc(-c4ccc(-c5ccc6c(c5)sc5ccccc56)c5oc6ccccc6c45)cc3)nc(-c3ccc(-c4cccc5ncccc45)cc3)n2)cc1 ; c1ccc(-c2cc(-c3ccc(-c4ccc(-c5ccc6c(c5)sc5ccccc56)c5oc6ccccc6c45)cc3)nc(-c3ccc(-c4cccc5cccnc45)cc3)n2)cc1 ; c1ccc(-c2cccc(-c3nc(-c4ccccc4)nc(-c4ccc(-c5ccc6sc7ccccc7c6c5)c5c4oc4ccccc45)n3)c2)cc1]

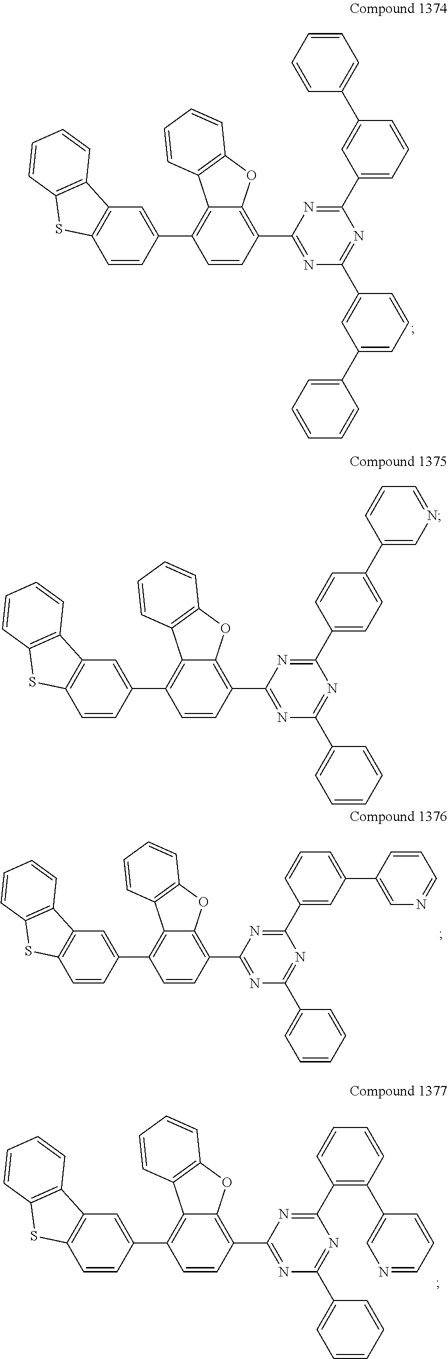 [SMILES: c1ccc(-c2cccc(-c3nc(-c4cccc(-c5ccccc5)c4)nc(-c4ccc(-c5ccc6sc7ccccc7c6c5)c5c4oc4ccccc45)n3)c2)cc1 ; c1ccc(-c2nc(-c3ccc(-c4cccnc4)cc3)nc(-c3ccc(-c4ccc5sc6ccccc6c5c4)c4c3oc3ccccc34)n2)cc1 ; c1ccc(-c2nc(-c3cccc(-c4cccnc4)c3)nc(-c3ccc(-c4ccc5sc6ccccc6c5c4)c4c3oc3ccccc34)n2)cc1 ; c1ccc(-c2nc(-c3ccccc3-c3cccnc3)nc(-c3ccc(-c4ccc5sc6ccccc6c5c4)c4c3oc3ccccc34)n2)cc1]

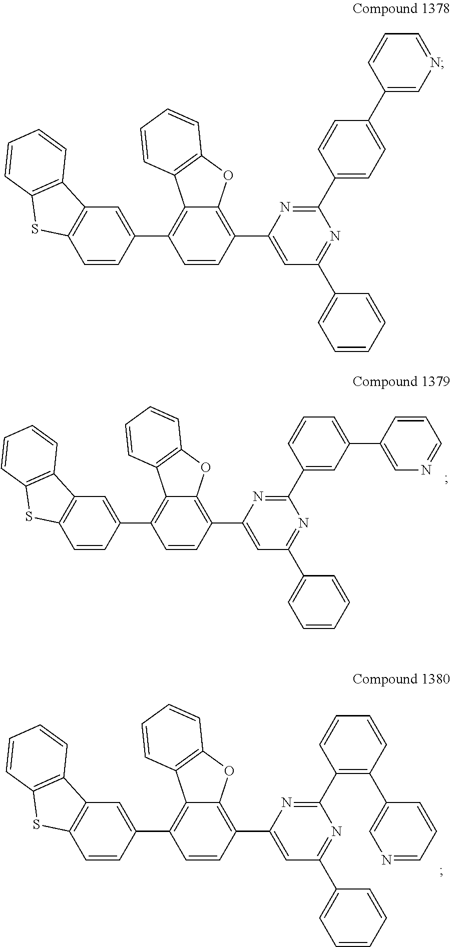 [SMILES: c1ccc(-c2cc(-c3ccc(-c4ccc5sc6ccccc6c5c4)c4c3oc3ccccc34)nc(-c3ccc(-c4cccnc4)cc3)n2)cc1 ; c1ccc(-c2cc(-c3ccc(-c4ccc5sc6ccccc6c5c4)c4c3oc3ccccc34)nc(-c3cccc(-c4cccnc4)c3)n2)cc1 ; c1ccc(-c2cc(-c3ccc(-c4ccc5sc6ccccc6c5c4)c4c3oc3ccccc34)nc(-c3ccccc3-c3cccnc3)n2)cc1]

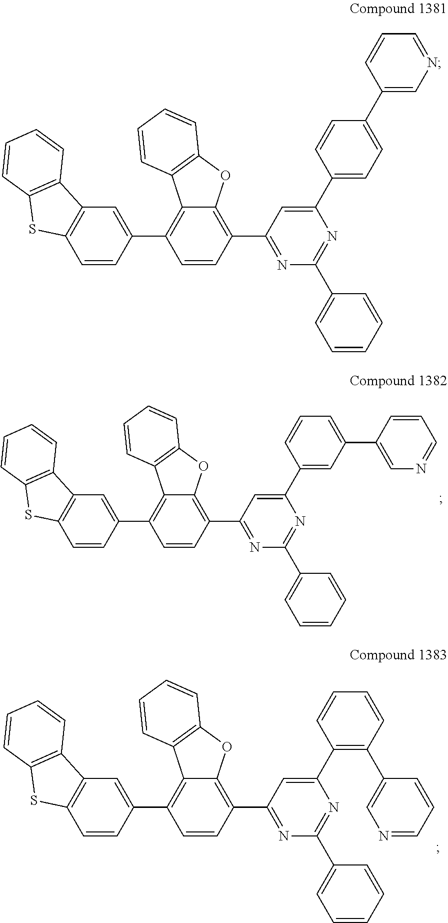 [SMILES: c1ccc(-c2nc(-c3ccc(-c4cccnc4)cc3)cc(-c3ccc(-c4ccc5sc6ccccc6c5c4)c4c3oc3ccccc34)n2)cc1 ; c1ccc(-c2nc(-c3cccc(-c4cccnc4)c3)cc(-c3ccc(-c4ccc5sc6ccccc6c5c4)c4c3oc3ccccc34)n2)cc1 ; c1ccc(-c2nc(-c3ccccc3-c3cccnc3)cc(-c3ccc(-c4ccc5sc6ccccc6c5c4)c4c3oc3ccccc34)n2)cc1]

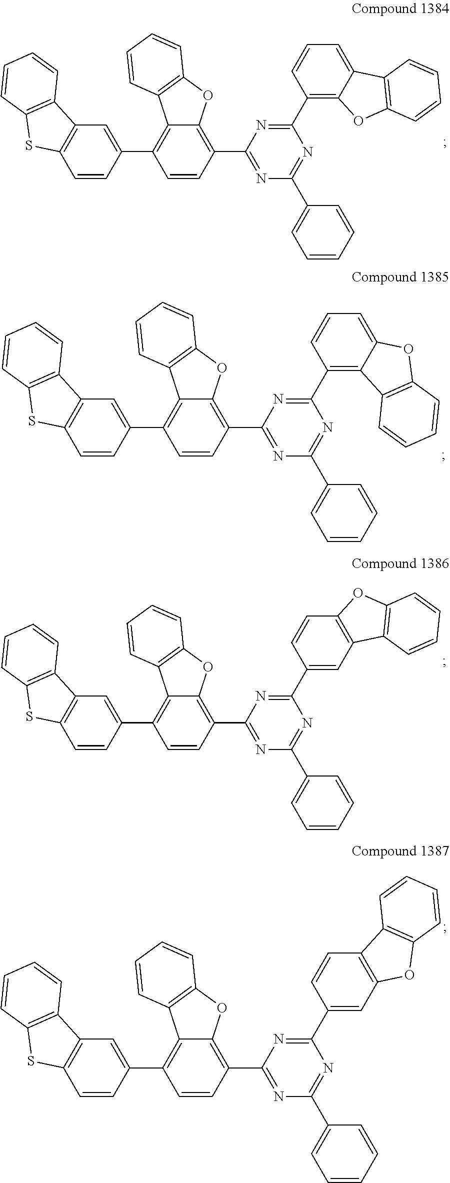 [SMILES: c1ccc(-c2nc(-c3cccc4c3oc3ccccc34)nc(-c3ccc(-c4ccc5sc6ccccc6c5c4)c4c3oc3ccccc34)n2)cc1 ; c1ccc(-c2nc(-c3ccc(-c4ccc5sc6ccccc6c5c4)c4c3oc3ccccc34)nc(-c3cccc4oc5ccccc5c34)n2)cc1 ; c1ccc(-c2nc(-c3ccc4oc5ccccc5c4c3)nc(-c3ccc(-c4ccc5sc6ccccc6c5c4)c4c3oc3ccccc34)n2)cc1 ; c1ccc(-c2nc(-c3ccc4c(c3)oc3ccccc34)nc(-c3ccc(-c4ccc5sc6ccccc6c5c4)c4c3oc3ccccc34)n2)cc1]

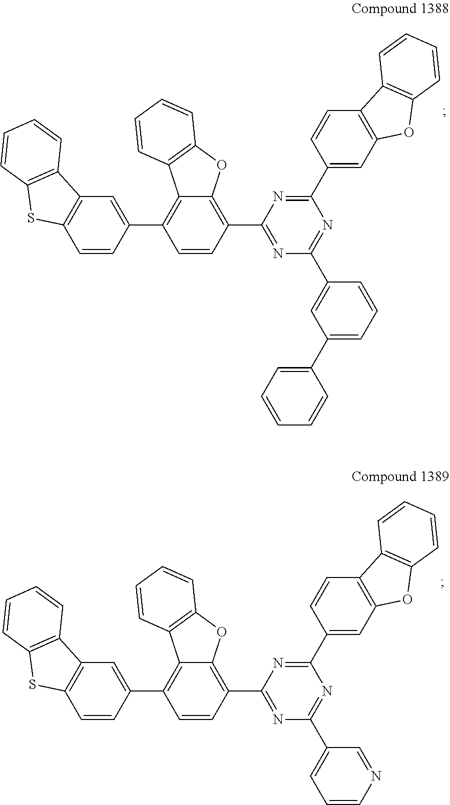 [SMILES: c1ccc(-c2cccc(-c3nc(-c4ccc5c(c4)oc4ccccc45)nc(-c4ccc(-c5ccc6sc7ccccc7c6c5)c5c4oc4ccccc45)n3)c2)cc1 ; c1cncc(-c2nc(-c3ccc4c(c3)oc3ccccc34)nc(-c3ccc(-c4ccc5sc6ccccc6c5c4)c4c3oc3ccccc34)n2)c1]

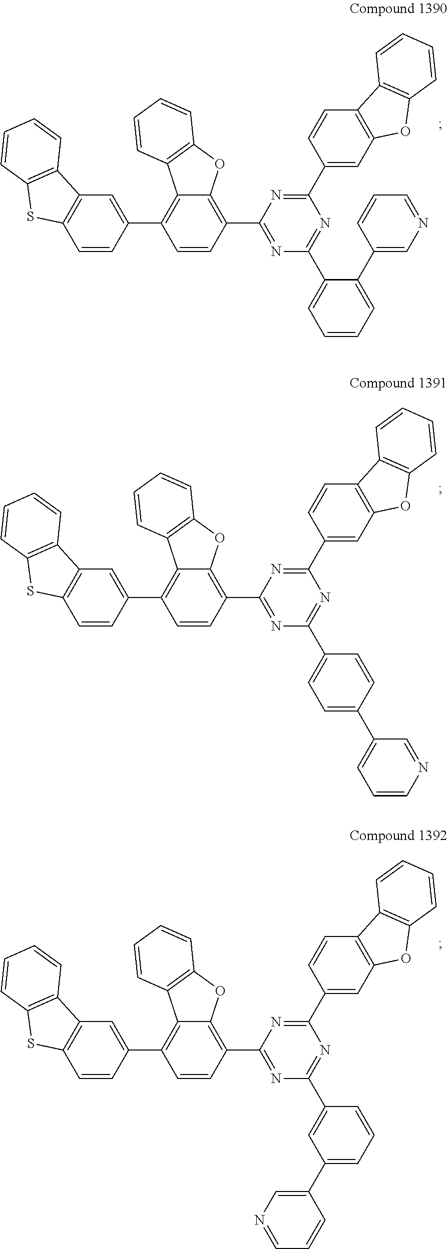 [SMILES: c1cncc(-c2ccccc2-c2nc(-c3ccc4c(c3)oc3ccccc34)nc(-c3ccc(-c4ccc5sc6ccccc6c5c4)c4c3oc3ccccc34)n2)c1 ; c1cncc(-c2ccc(-c3nc(-c4ccc5c(c4)oc4ccccc45)nc(-c4ccc(-c5ccc6sc7ccccc7c6c5)c5c4oc4ccccc45)n3)cc2)c1 ; c1cncc(-c2cccc(-c3nc(-c4ccc5c(c4)oc4ccccc45)nc(-c4ccc(-c5ccc6sc7ccccc7c6c5)c5c4oc4ccccc45)n3)c2)c1]

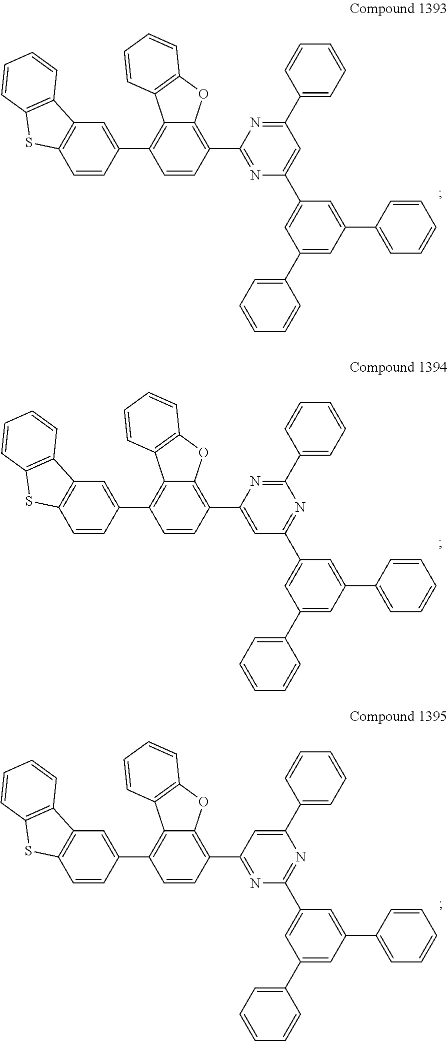 [SMILES: c1ccc(-c2cc(-c3ccccc3)cc(-c3cc(-c4ccccc4)nc(-c4ccc(-c5ccc6sc7ccccc7c6c5)c5c4oc4ccccc45)n3)c2)cc1 ; c1ccc(-c2cc(-c3ccccc3)cc(-c3cc(-c4ccc(-c5ccc6sc7ccccc7c6c5)c5c4oc4ccccc45)nc(-c4ccccc4)n3)c2)cc1 ; c1ccc(-c2cc(-c3ccccc3)cc(-c3nc(-c4ccccc4)cc(-c4ccc(-c5ccc6sc7ccccc7c6c5)c5c4oc4ccccc45)n3)c2)cc1]

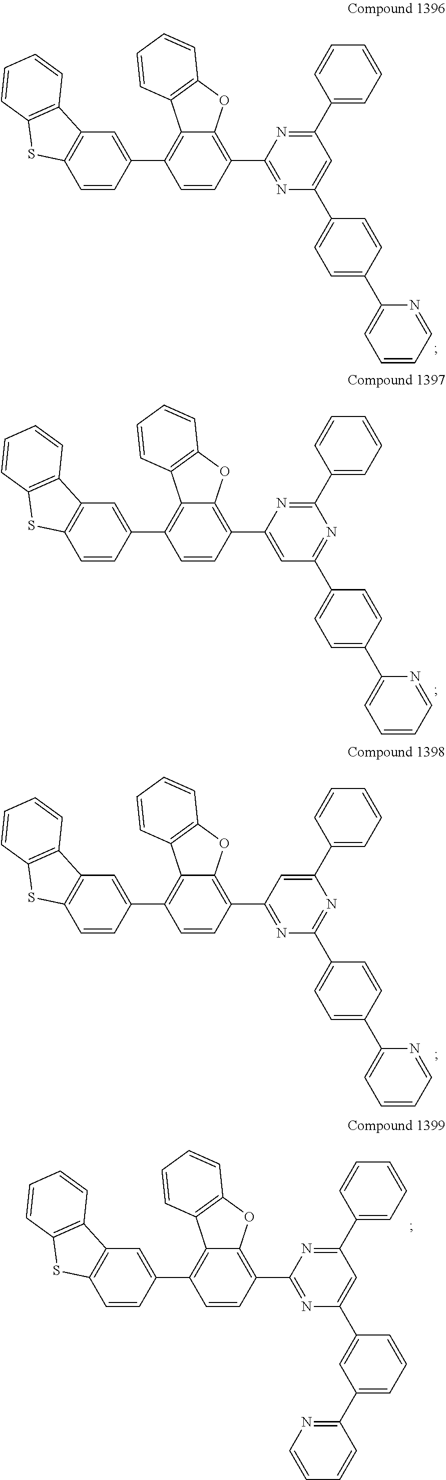 [SMILES: c1ccc(-c2cc(-c3ccc(-c4ccccn4)cc3)nc(-c3ccc(-c4ccc5sc6ccccc6c5c4)c4c3oc3ccccc34)n2)cc1 ; c1ccc(-c2nc(-c3ccc(-c4ccccn4)cc3)cc(-c3ccc(-c4ccc5sc6ccccc6c5c4)c4c3oc3ccccc34)n2)cc1 ; c1ccc(-c2cc(-c3ccc(-c4ccc5sc6ccccc6c5c4)c4c3oc3ccccc34)nc(-c3ccc(-c4ccccn4)cc3)n2)cc1 ; c1ccc(-c2cc(-c3cccc(-c4ccccn4)c3)nc(-c3ccc(-c4ccc5sc6ccccc6c5c4)c4c3oc3ccccc34)n2)cc1]

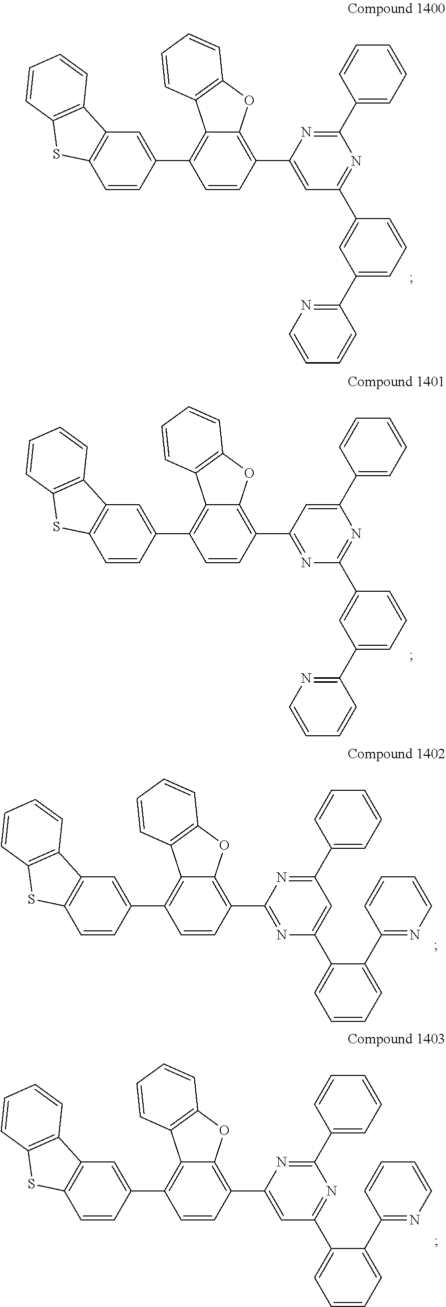 [SMILES: c1ccc(-c2nc(-c3cccc(-c4ccccn4)c3)cc(-c3ccc(-c4ccc5sc6ccccc6c5c4)c4c3oc3ccccc34)n2)cc1 ; c1ccc(-c2cc(-c3ccc(-c4ccc5sc6ccccc6c5c4)c4c3oc3ccccc34)nc(-c3cccc(-c4ccccn4)c3)n2)cc1 ; c1ccc(-c2cc(-c3ccccc3-c3ccccn3)nc(-c3ccc(-c4ccc5sc6ccccc6c5c4)c4c3oc3ccccc34)n2)cc1 ; c1ccc(-c2nc(-c3ccccc3-c3ccccn3)cc(-c3ccc(-c4ccc5sc6ccccc6c5c4)c4c3oc3ccccc34)n2)cc1]

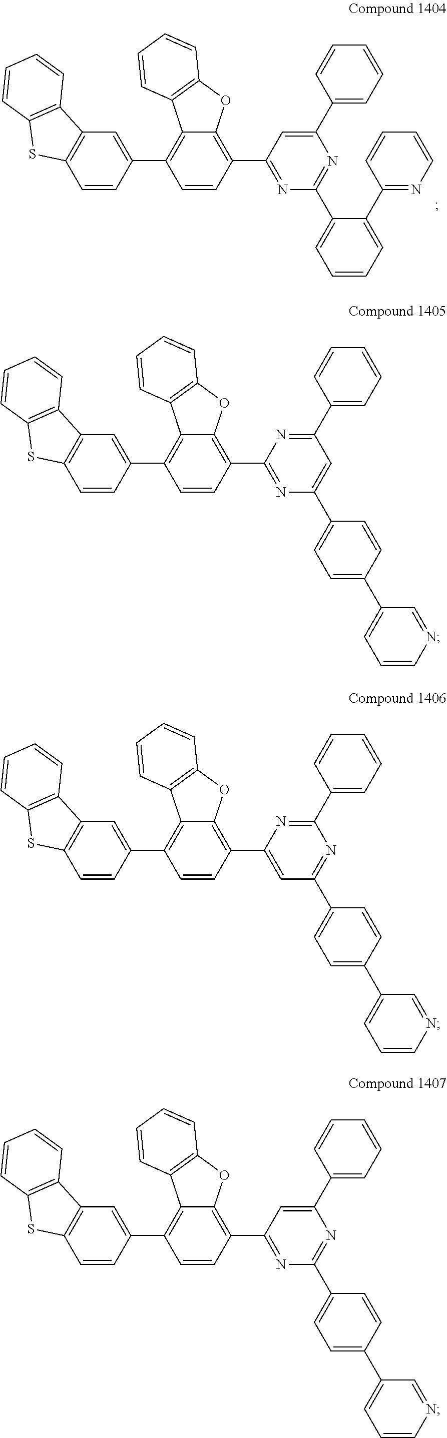 [SMILES: c1ccc(-c2cc(-c3ccc(-c4ccc5sc6ccccc6c5c4)c4c3oc3ccccc34)nc(-c3ccccc3-c3ccccn3)n2)cc1 ; c1ccc(-c2cc(-c3ccc(-c4cccnc4)cc3)nc(-c3ccc(-c4ccc5sc6ccccc6c5c4)c4c3oc3ccccc34)n2)cc1 ; c1ccc(-c2nc(-c3ccc(-c4cccnc4)cc3)cc(-c3ccc(-c4ccc5sc6ccccc6c5c4)c4c3oc3ccccc34)n2)cc1 ; c1ccc(-c2cc(-c3ccc(-c4ccc5sc6ccccc6c5c4)c4c3oc3ccccc34)nc(-c3ccc(-c4cccnc4)cc3)n2)cc1]

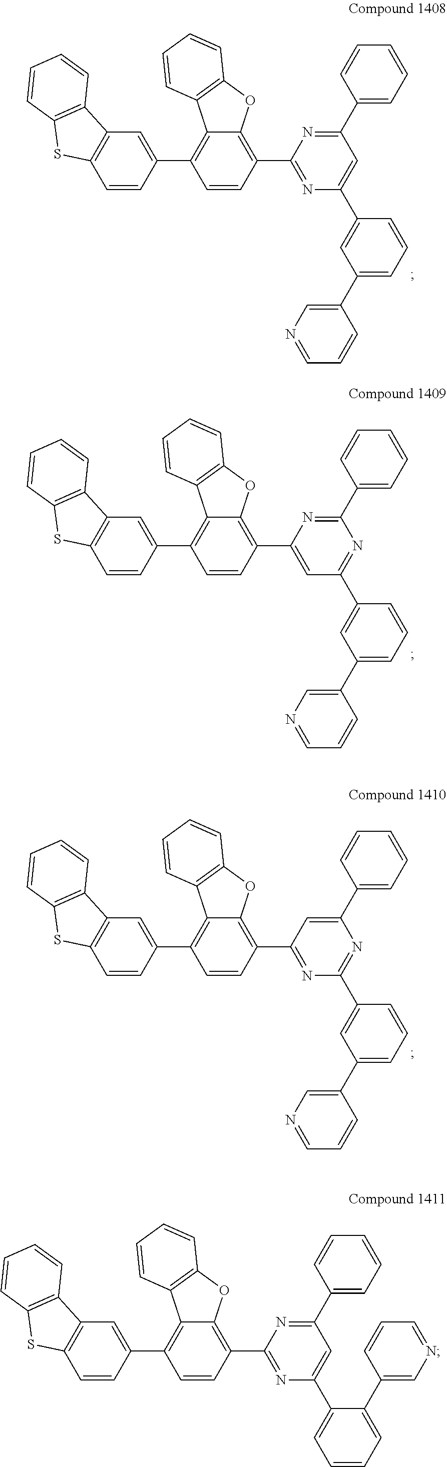 [SMILES: c1ccc(-c2cc(-c3cccc(-c4cccnc4)c3)nc(-c3ccc(-c4ccc5sc6ccccc6c5c4)c4c3oc3ccccc34)n2)cc1 ; c1ccc(-c2nc(-c3cccc(-c4cccnc4)c3)cc(-c3ccc(-c4ccc5sc6ccccc6c5c4)c4c3oc3ccccc34)n2)cc1 ; c1ccc(-c2cc(-c3ccc(-c4ccc5sc6ccccc6c5c4)c4c3oc3ccccc34)nc(-c3cccc(-c4cccnc4)c3)n2)cc1 ; c1ccc(-c2cc(-c3ccccc3-c3cccnc3)nc(-c3ccc(-c4ccc5sc6ccccc6c5c4)c4c3oc3ccccc34)n2)cc1]

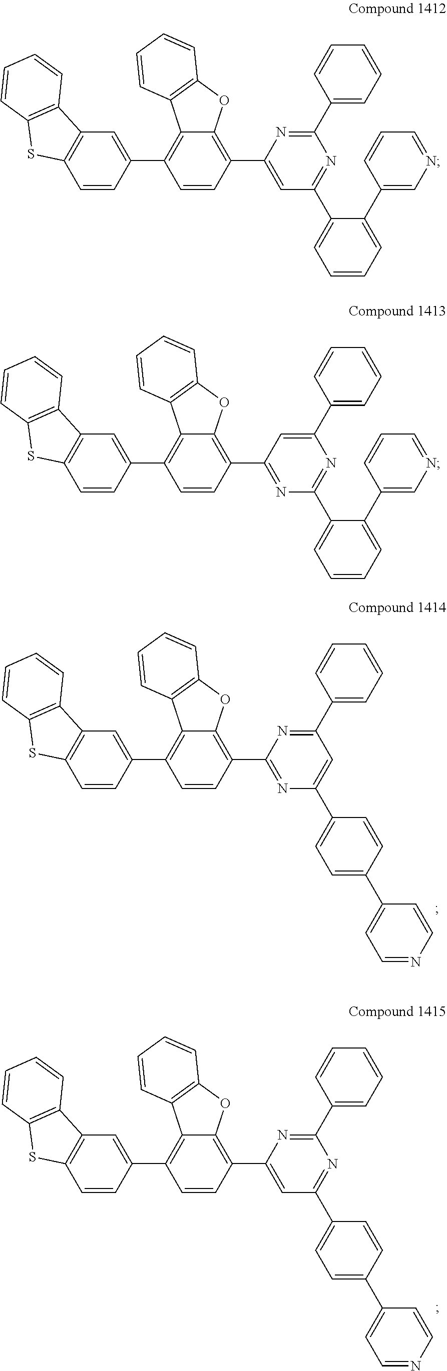 [SMILES: c1ccc(-c2nc(-c3ccccc3-c3cccnc3)cc(-c3ccc(-c4ccc5sc6ccccc6c5c4)c4c3oc3ccccc34)n2)cc1 ; c1ccc(-c2cc(-c3ccc(-c4ccc5sc6ccccc6c5c4)c4c3oc3ccccc34)nc(-c3ccccc3-c3cccnc3)n2)cc1 ; c1ccc(-c2cc(-c3ccc(-c4ccncc4)cc3)nc(-c3ccc(-c4ccc5sc6ccccc6c5c4)c4c3oc3ccccc34)n2)cc1 ; c1ccc(-c2nc(-c3ccc(-c4ccncc4)cc3)cc(-c3ccc(-c4ccc5sc6ccccc6c5c4)c4c3oc3ccccc34)n2)cc1]

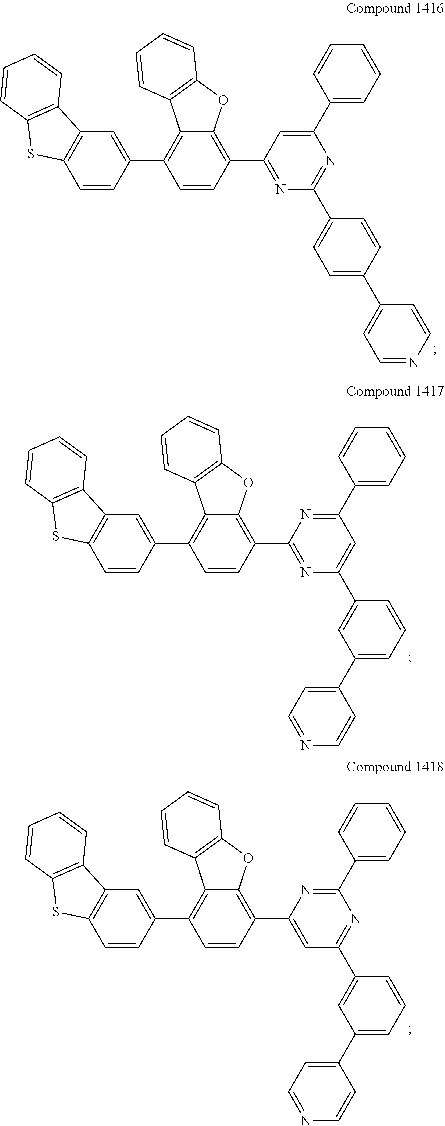 [SMILES: c1ccc(-c2cc(-c3ccc(-c4ccc5sc6ccccc6c5c4)c4c3oc3ccccc34)nc(-c3ccc(-c4ccncc4)cc3)n2)cc1 ; c1ccc(-c2cc(-c3cccc(-c4ccncc4)c3)nc(-c3ccc(-c4ccc5sc6ccccc6c5c4)c4c3oc3ccccc34)n2)cc1 ; c1ccc(-c2nc(-c3cccc(-c4ccncc4)c3)cc(-c3ccc(-c4ccc5sc6ccccc6c5c4)c4c3oc3ccccc34)n2)cc1]

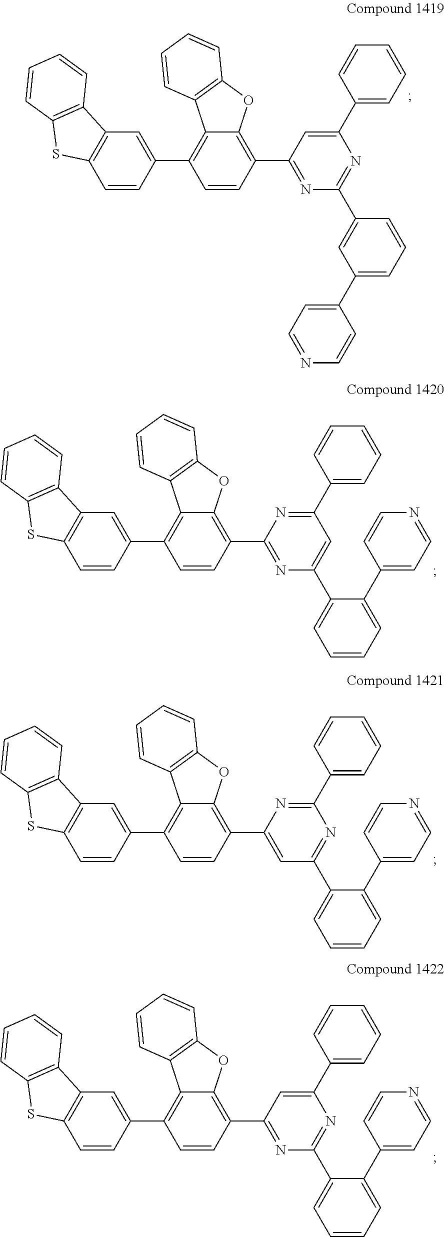 [SMILES: c1ccc(-c2cc(-c3ccc(-c4ccc5sc6ccccc6c5c4)c4c3oc3ccccc34)nc(-c3cccc(-c4ccncc4)c3)n2)cc1 ; c1ccc(-c2cc(-c3ccccc3-c3ccncc3)nc(-c3ccc(-c4ccc5sc6ccccc6c5c4)c4c3oc3ccccc34)n2)cc1 ; c1ccc(-c2nc(-c3ccccc3-c3ccncc3)cc(-c3ccc(-c4ccc5sc6ccccc6c5c4)c4c3oc3ccccc34)n2)cc1 ; c1ccc(-c2cc(-c3ccc(-c4ccc5sc6ccccc6c5c4)c4c3oc3ccccc34)nc(-c3ccccc3-c3ccncc3)n2)cc1]

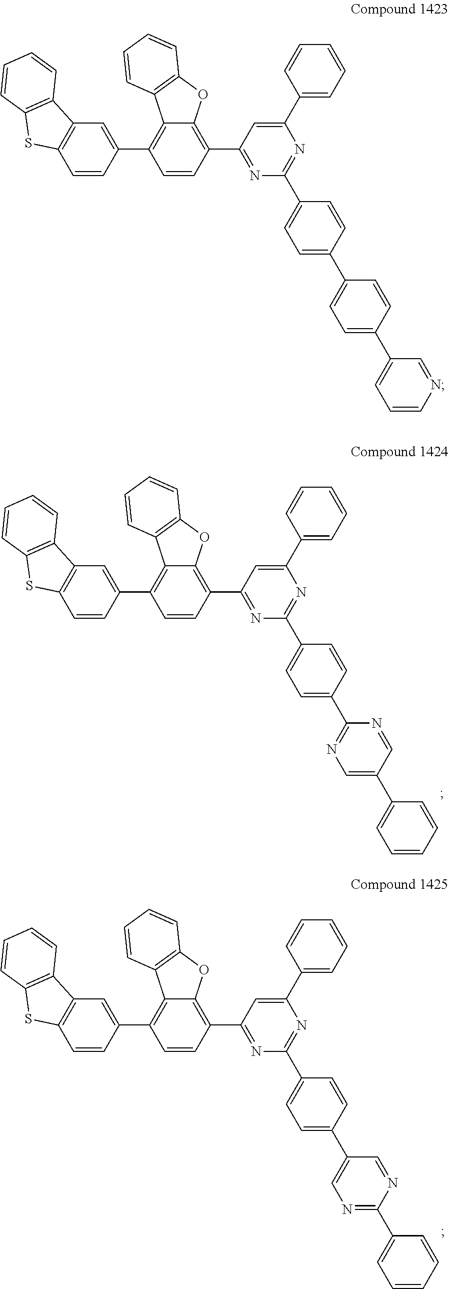 [SMILES: c1ccc(-c2cc(-c3ccc(-c4ccc5sc6ccccc6c5c4)c4c3oc3ccccc34)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc7sc8ccccc8c7c6)c6c5oc5ccccc56)n4)cc3)nc2)cc1 ; c1ccc(-c2cc(-c3ccc(-c4ccc5sc6ccccc6c5c4)c4c3oc3ccccc34)nc(-c3ccc(-c4cnc(-c5ccccc5)nc4)cc3)n2)cc1]

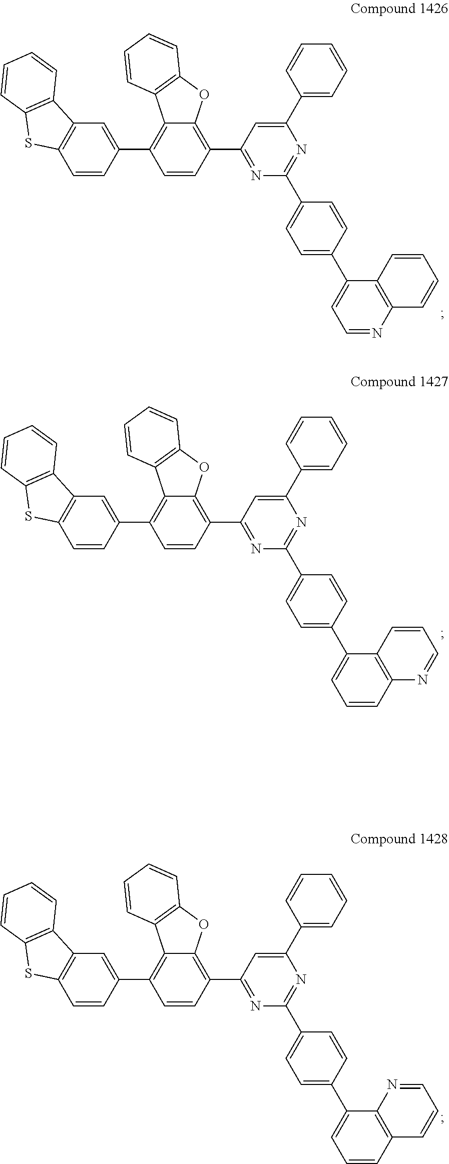 [SMILES: c1ccc(-c2cc(-c3ccc(-c4ccc5sc6ccccc6c5c4)c4c3oc3ccccc34)nc(-c3ccc(-c4ccnc5ccccc45)cc3)n2)cc1 ; c1ccc(-c2cc(-c3ccc(-c4ccc5sc6ccccc6c5c4)c4c3oc3ccccc34)nc(-c3ccc(-c4cccc5ncccc45)cc3)n2)cc1 ; c1ccc(-c2cc(-c3ccc(-c4ccc5sc6ccccc6c5c4)c4c3oc3ccccc34)nc(-c3ccc(-c4cccc5cccnc45)cc3)n2)cc1]

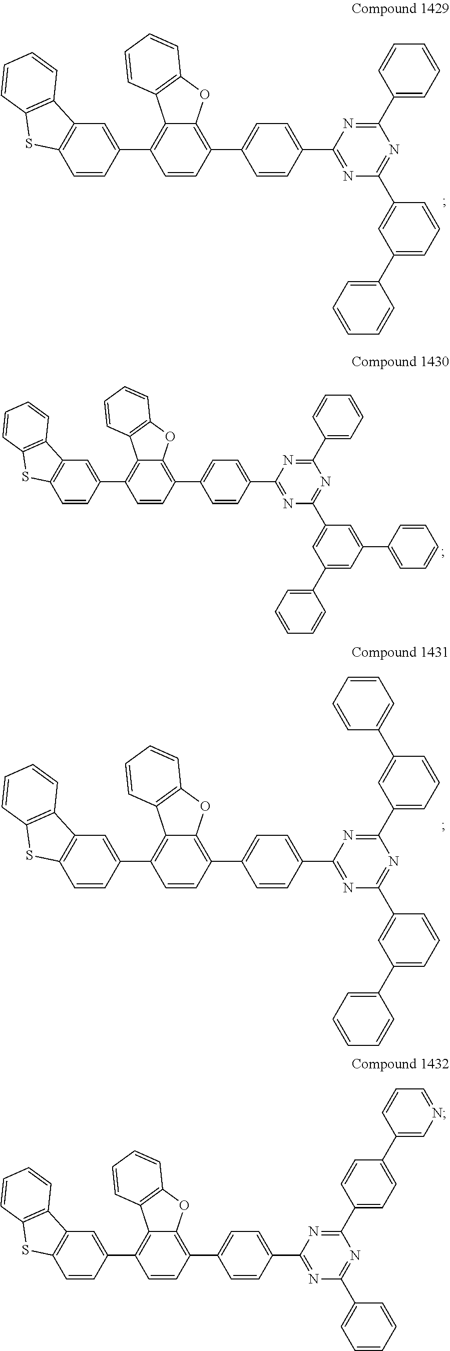 [SMILES: c1ccc(-c2cccc(-c3nc(-c4ccccc4)nc(-c4ccc(-c5ccc(-c6ccc7sc8ccccc8c7c6)c6c5oc5ccccc56)cc4)n3)c2)cc1 ; c1ccc(-c2cc(-c3ccccc3)cc(-c3nc(-c4ccccc4)nc(-c4ccc(-c5ccc(-c6ccc7sc8ccccc8c7c6)c6c5oc5ccccc56)cc4)n3)c2)cc1 ; c1ccc(-c2cccc(-c3nc(-c4ccc(-c5ccc(-c6ccc7sc8ccccc8c7c6)c6c5oc5ccccc56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5ccc6sc7ccccc7c6c5)c5c4oc4ccccc45)cc3)n2)cc1]

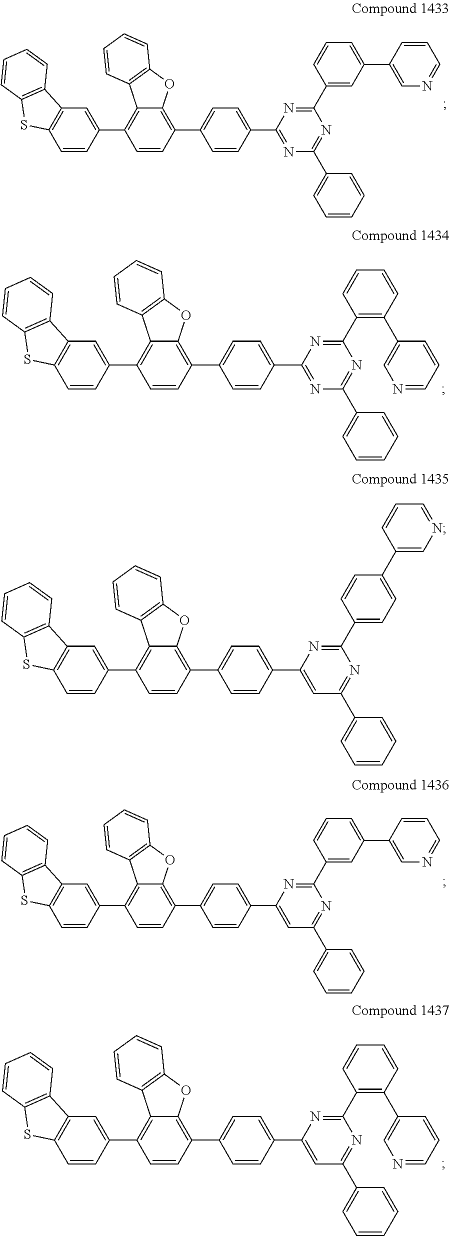 [SMILES: c1ccc(-c2nc(-c3ccc(-c4ccc(-c5ccc6sc7ccccc7c6c5)c5c4oc4ccccc45)cc3)nc(-c3cccc(-c4cccnc4)c3)n2)cc1 ; c1ccc(-c2nc(-c3ccc(-c4ccc(-c5ccc6sc7ccccc7c6c5)c5c4oc4ccccc45)cc3)nc(-c3ccccc3-c3cccnc3)n2)cc1 ; c1ccc(-c2cc(-c3ccc(-c4ccc(-c5ccc6sc7ccccc7c6c5)c5c4oc4ccccc45)cc3)nc(-c3ccc(-c4cccnc4)cc3)n2)cc1 ; c1ccc(-c2cc(-c3ccc(-c4ccc(-c5ccc6sc7ccccc7c6c5)c5c4oc4ccccc45)cc3)nc(-c3cccc(-c4cccnc4)c3)n2)cc1 ; c1ccc(-c2cc(-c3ccc(-c4ccc(-c5ccc6sc7ccccc7c6c5)c5c4oc4ccccc45)cc3)nc(-c3ccccc3-c3cccnc3)n2)cc1]

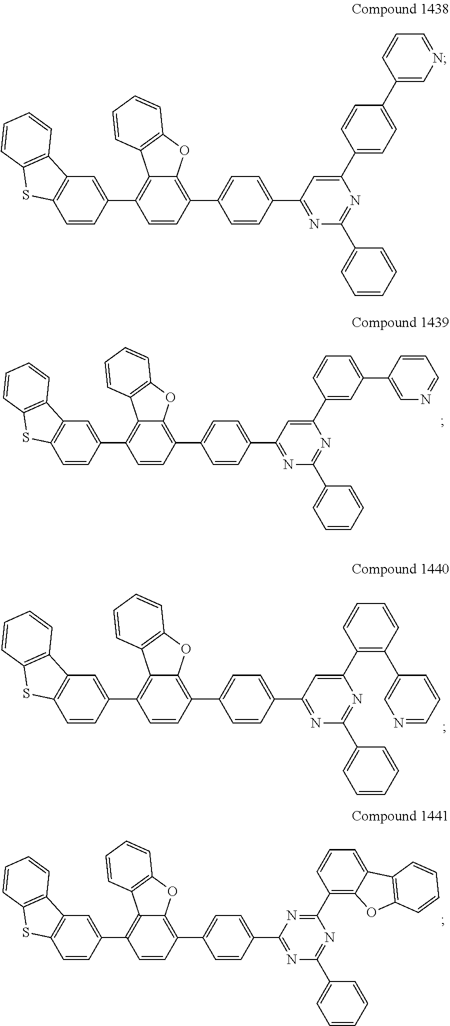 [SMILES: c1ccc(-c2nc(-c3ccc(-c4cccnc4)cc3)cc(-c3ccc(-c4ccc(-c5ccc6sc7ccccc7c6c5)c5c4oc4ccccc45)cc3)n2)cc1 ; c1ccc(-c2nc(-c3ccc(-c4ccc(-c5ccc6sc7ccccc7c6c5)c5c4oc4ccccc45)cc3)cc(-c3cccc(-c4cccnc4)c3)n2)cc1 ; c1ccc(-c2nc(-c3ccc(-c4ccc(-c5ccc6sc7ccccc7c6c5)c5c4oc4ccccc45)cc3)cc(-c3ccccc3-c3cccnc3)n2)cc1 ; c1ccc(-c2nc(-c3ccc(-c4ccc(-c5ccc6sc7ccccc7c6c5)c5c4oc4ccccc45)cc3)nc(-c3cccc4c3oc3ccccc34)n2)cc1]

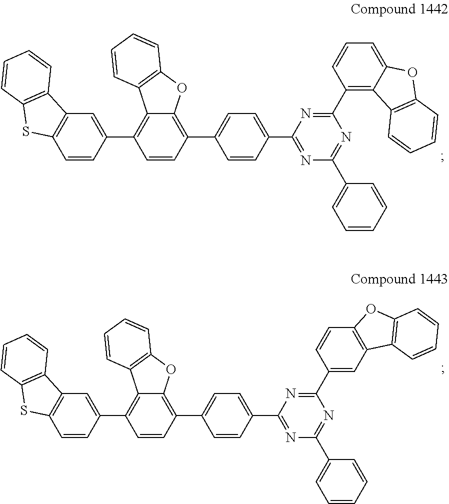 [SMILES: c1ccc(-c2nc(-c3ccc(-c4ccc(-c5ccc6sc7ccccc7c6c5)c5c4oc4ccccc45)cc3)nc(-c3cccc4oc5ccccc5c34)n2)cc1 ; c1ccc(-c2nc(-c3ccc(-c4ccc(-c5ccc6sc7ccccc7c6c5)c5c4oc4ccccc45)cc3)nc(-c3ccc4oc5ccccc5c4c3)n2)cc1]

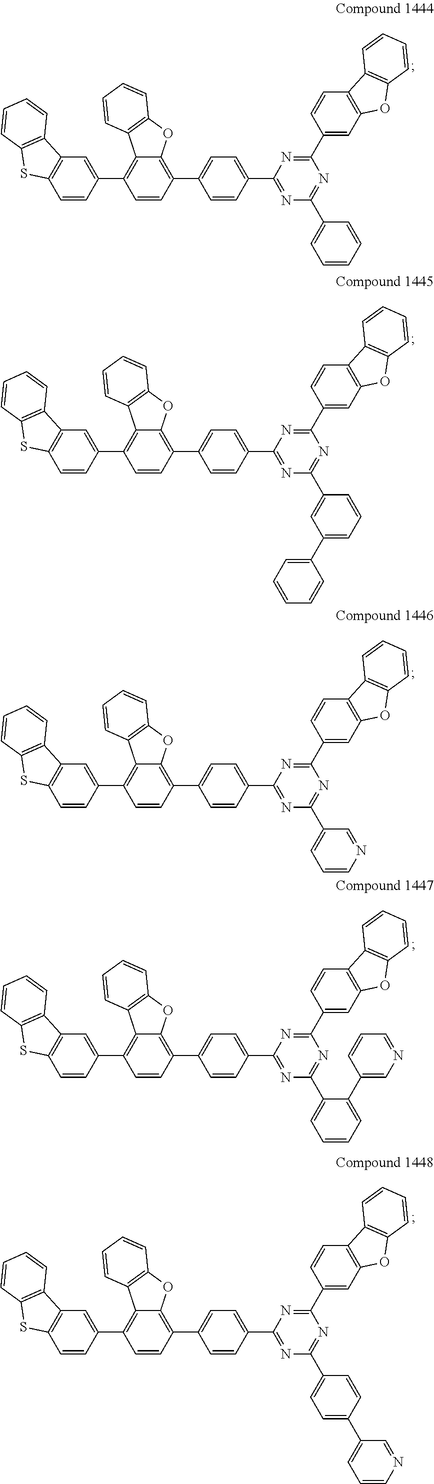

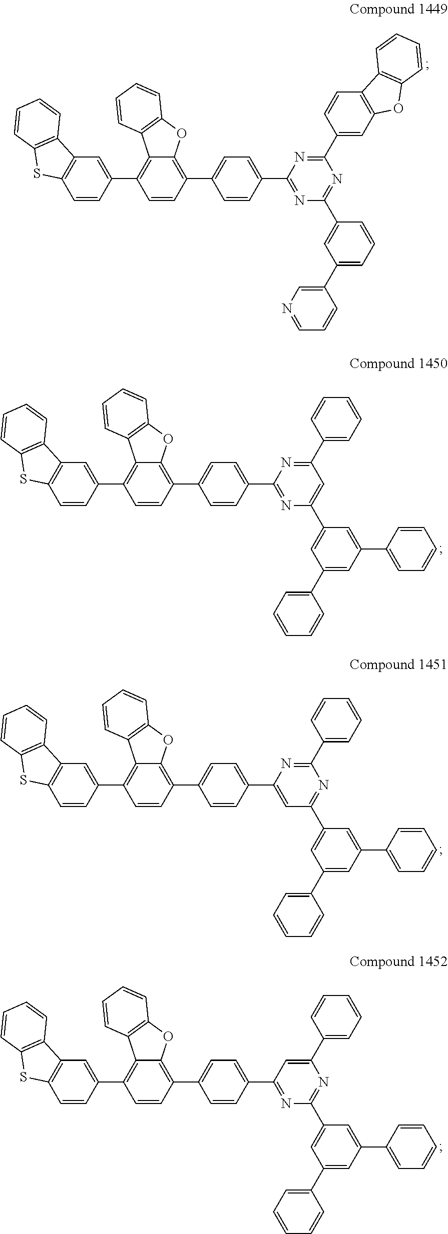 [SMILES: c1cncc(-c2cccc(-c3nc(-c4ccc(-c5ccc(-c6ccc7sc8ccccc8c7c6)c6c5oc5ccccc56)cc4)nc(-c4ccc5c(c4)oc4ccccc45)n3)c2)c1 ; c1ccc(-c2cc(-c3ccccc3)cc(-c3cc(-c4ccccc4)nc(-c4ccc(-c5ccc(-c6ccc7sc8ccccc8c7c6)c6c5oc5ccccc56)cc4)n3)c2)cc1 ; c1ccc(-c2cc(-c3ccccc3)cc(-c3cc(-c4ccc(-c5ccc(-c6ccc7sc8ccccc8c7c6)c6c5oc5ccccc56)cc4)nc(-c4ccccc4)n3)c2)cc1 ; c1ccc(-c2cc(-c3ccccc3)cc(-c3nc(-c4ccccc4)cc(-c4ccc(-c5ccc(-c6ccc7sc8ccccc8c7c6)c6c5oc5ccccc56)cc4)n3)c2)cc1]

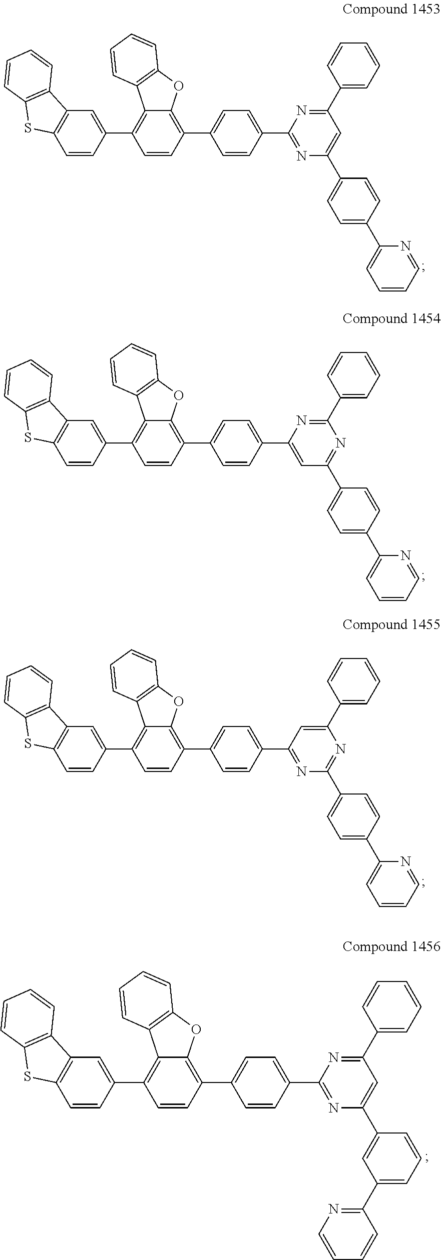 [SMILES: c1ccc(-c2cc(-c3ccc(-c4ccccn4)cc3)nc(-c3ccc(-c4ccc(-c5ccc6sc7ccccc7c6c5)c5c4oc4ccccc45)cc3)n2)cc1 ; c1ccc(-c2nc(-c3ccc(-c4ccccn4)cc3)cc(-c3ccc(-c4ccc(-c5ccc6sc7ccccc7c6c5)c5c4oc4ccccc45)cc3)n2)cc1 ; c1ccc(-c2cc(-c3ccc(-c4ccc(-c5ccc6sc7ccccc7c6c5)c5c4oc4ccccc45)cc3)nc(-c3ccc(-c4ccccn4)cc3)n2)cc1 ; c1ccc(-c2cc(-c3cccc(-c4ccccn4)c3)nc(-c3ccc(-c4ccc(-c5ccc6sc7ccccc7c6c5)c5c4oc4ccccc45)cc3)n2)cc1]

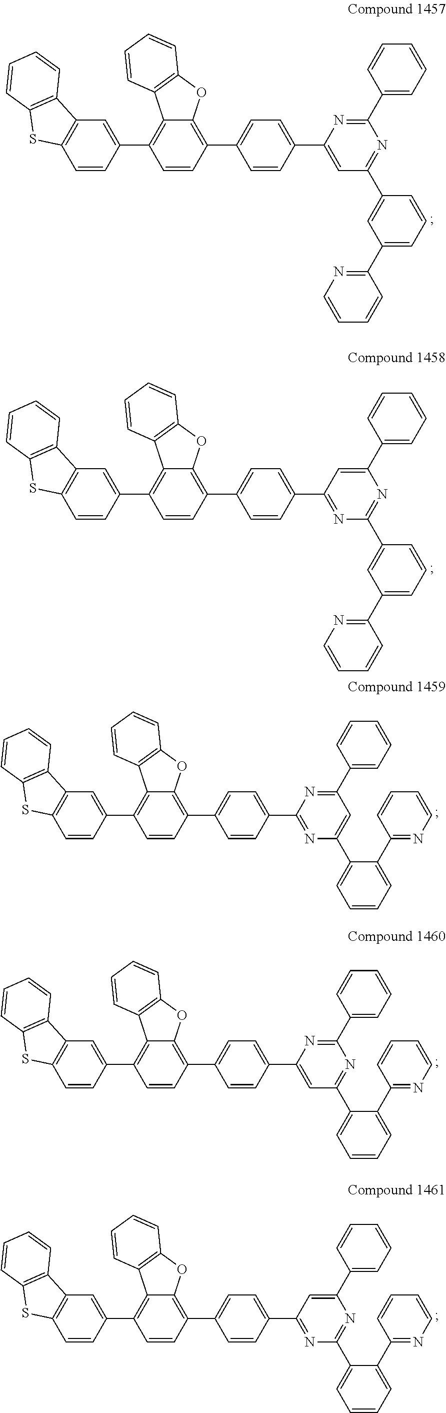 [SMILES: c1ccc(-c2nc(-c3ccc(-c4ccc(-c5ccc6sc7ccccc7c6c5)c5c4oc4ccccc45)cc3)cc(-c3cccc(-c4ccccn4)c3)n2)cc1 ; c1ccc(-c2cc(-c3ccc(-c4ccc(-c5ccc6sc7ccccc7c6c5)c5c4oc4ccccc45)cc3)nc(-c3cccc(-c4ccccn4)c3)n2)cc1 ; c1ccc(-c2cc(-c3ccccc3-c3ccccn3)nc(-c3ccc(-c4ccc(-c5ccc6sc7ccccc7c6c5)c5c4oc4ccccc45)cc3)n2)cc1 ; c1ccc(-c2nc(-c3ccc(-c4ccc(-c5ccc6sc7ccccc7c6c5)c5c4oc4ccccc45)cc3)cc(-c3ccccc3-c3ccccn3)n2)cc1 ; c1ccc(-c2cc(-c3ccc(-c4ccc(-c5ccc6sc7ccccc7c6c5)c5c4oc4ccccc45)cc3)nc(-c3ccccc3-c3ccccn3)n2)cc1]

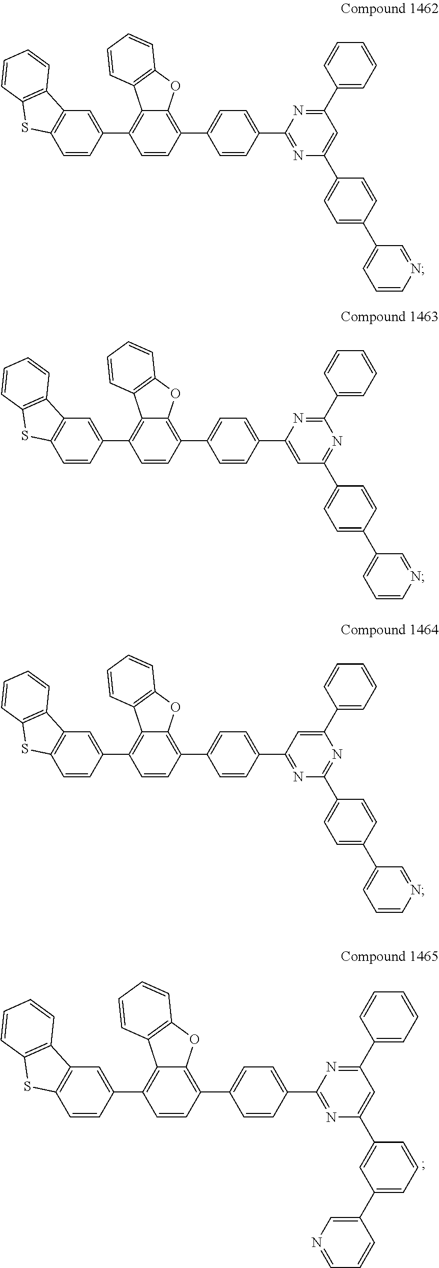 [SMILES: c1ccc(-c2cc(-c3ccc(-c4cccnc4)cc3)nc(-c3ccc(-c4ccc(-c5ccc6sc7ccccc7c6c5)c5c4oc4ccccc45)cc3)n2)cc1 ; c1ccc(-c2nc(-c3ccc(-c4cccnc4)cc3)cc(-c3ccc(-c4ccc(-c5ccc6sc7ccccc7c6c5)c5c4oc4ccccc45)cc3)n2)cc1 ; c1ccc(-c2cc(-c3ccc(-c4ccc(-c5ccc6sc7ccccc7c6c5)c5c4oc4ccccc45)cc3)nc(-c3ccc(-c4cccnc4)cc3)n2)cc1 ; c1ccc(-c2cc(-c3cccc(-c4cccnc4)c3)nc(-c3ccc(-c4ccc(-c5ccc6sc7ccccc7c6c5)c5c4oc4ccccc45)cc3)n2)cc1]

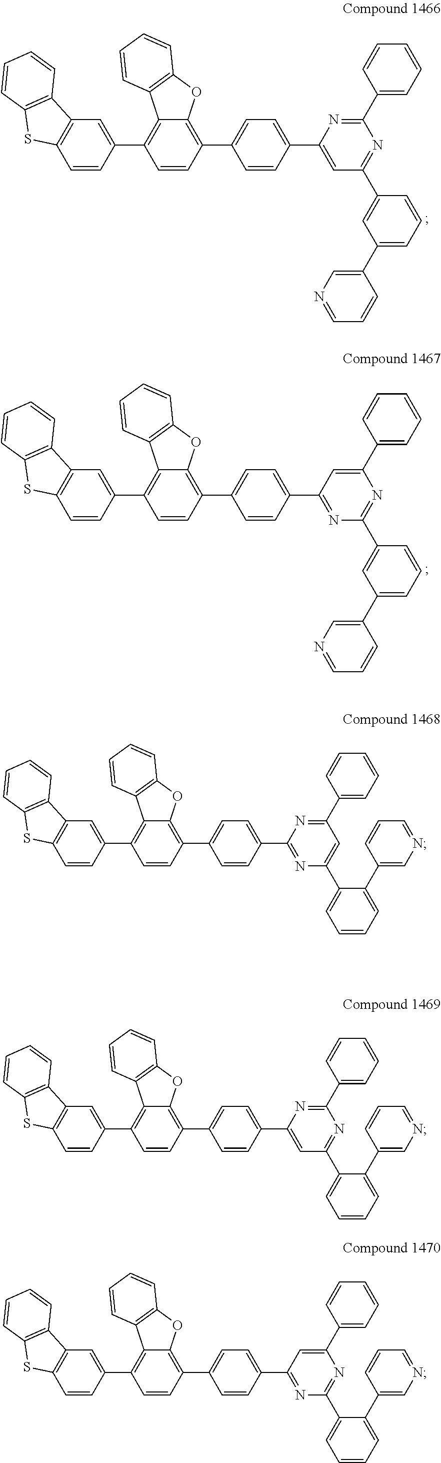 [SMILES: c1ccc(-c2nc(-c3ccc(-c4ccc(-c5ccc6sc7ccccc7c6c5)c5c4oc4ccccc45)cc3)cc(-c3cccc(-c4cccnc4)c3)n2)cc1 ; c1ccc(-c2cc(-c3ccc(-c4ccc(-c5ccc6sc7ccccc7c6c5)c5c4oc4ccccc45)cc3)nc(-c3cccc(-c4cccnc4)c3)n2)cc1 ; c1ccc(-c2cc(-c3ccccc3-c3cccnc3)nc(-c3ccc(-c4ccc(-c5ccc6sc7ccccc7c6c5)c5c4oc4ccccc45)cc3)n2)cc1 ; c1ccc(-c2nc(-c3ccc(-c4ccc(-c5ccc6sc7ccccc7c6c5)c5c4oc4ccccc45)cc3)cc(-c3ccccc3-c3cccnc3)n2)cc1 ; c1ccc(-c2cc(-c3ccc(-c4ccc(-c5ccc6sc7ccccc7c6c5)c5c4oc4ccccc45)cc3)nc(-c3ccccc3-c3cccnc3)n2)cc1]

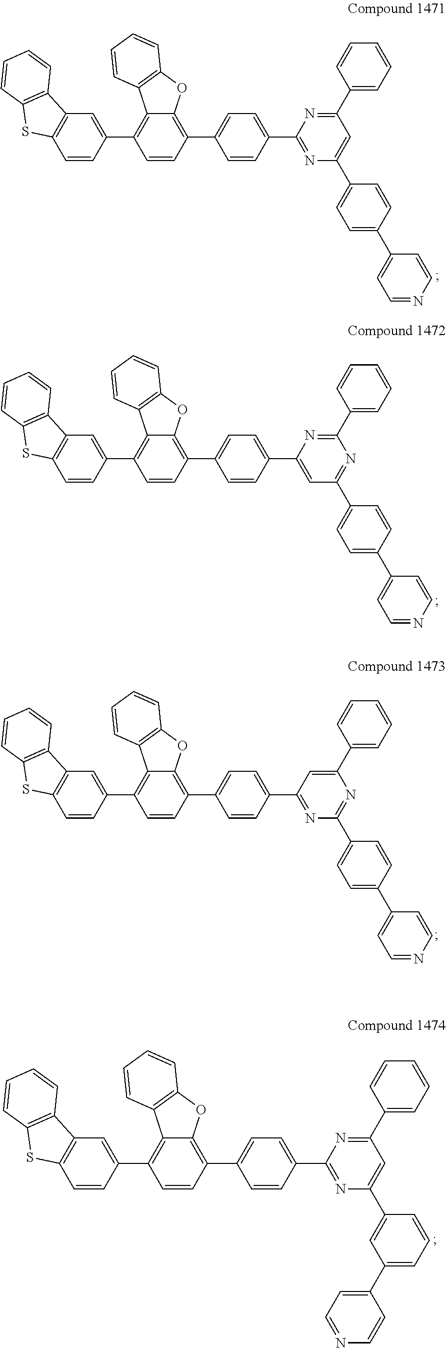 [SMILES: c1ccc(-c2cc(-c3ccc(-c4ccncc4)cc3)nc(-c3ccc(-c4ccc(-c5ccc6sc7ccccc7c6c5)c5c4oc4ccccc45)cc3)n2)cc1 ; c1ccc(-c2nc(-c3ccc(-c4ccncc4)cc3)cc(-c3ccc(-c4ccc(-c5ccc6sc7ccccc7c6c5)c5c4oc4ccccc45)cc3)n2)cc1 ; c1ccc(-c2cc(-c3ccc(-c4ccc(-c5ccc6sc7ccccc7c6c5)c5c4oc4ccccc45)cc3)nc(-c3ccc(-c4ccncc4)cc3)n2)cc1 ; c1ccc(-c2cc(-c3cccc(-c4ccncc4)c3)nc(-c3ccc(-c4ccc(-c5ccc6sc7ccccc7c6c5)c5c4oc4ccccc45)cc3)n2)cc1]

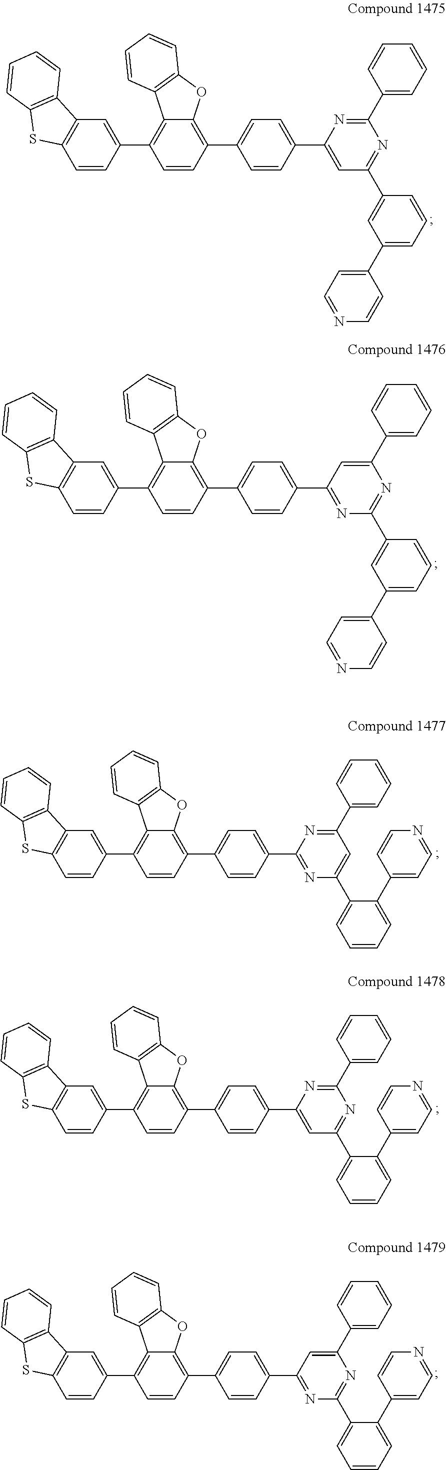 [SMILES: c1ccc(-c2nc(-c3ccc(-c4ccc(-c5ccc6sc7ccccc7c6c5)c5c4oc4ccccc45)cc3)cc(-c3cccc(-c4ccncc4)c3)n2)cc1 ; c1ccc(-c2cc(-c3ccc(-c4ccc(-c5ccc6sc7ccccc7c6c5)c5c4oc4ccccc45)cc3)nc(-c3cccc(-c4ccncc4)c3)n2)cc1 ; c1ccc(-c2cc(-c3ccccc3-c3ccncc3)nc(-c3ccc(-c4ccc(-c5ccc6sc7ccccc7c6c5)c5c4oc4ccccc45)cc3)n2)cc1 ; c1ccc(-c2nc(-c3ccc(-c4ccc(-c5ccc6sc7ccccc7c6c5)c5c4oc4ccccc45)cc3)cc(-c3ccccc3-c3ccncc3)n2)cc1 ; c1ccc(-c2cc(-c3ccc(-c4ccc(-c5ccc6sc7ccccc7c6c5)c5c4oc4ccccc45)cc3)nc(-c3ccccc3-c3ccncc3)n2)cc1]

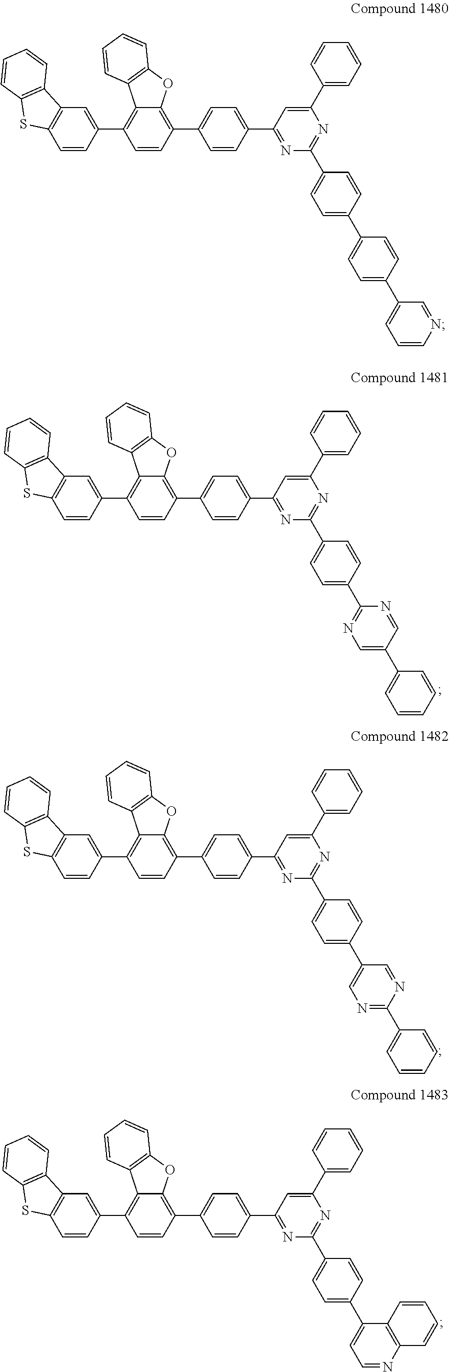 [SMILES: c1ccc(-c2cc(-c3ccc(-c4ccc(-c5ccc6sc7ccccc7c6c5)c5c4oc4ccccc45)cc3)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc(-c7ccc8sc9ccccc9c8c7)c7c6oc6ccccc67)cc5)n4)cc3)nc2)cc1 ; c1ccc(-c2cc(-c3ccc(-c4ccc(-c5ccc6sc7ccccc7c6c5)c5c4oc4ccccc45)cc3)nc(-c3ccc(-c4cnc(-c5ccccc5)nc4)cc3)n2)cc1 ; c1ccc(-c2cc(-c3ccc(-c4ccc(-c5ccc6sc7ccccc7c6c5)c5c4oc4ccccc45)cc3)nc(-c3ccc(-c4ccnc5ccccc45)cc3)n2)cc1]

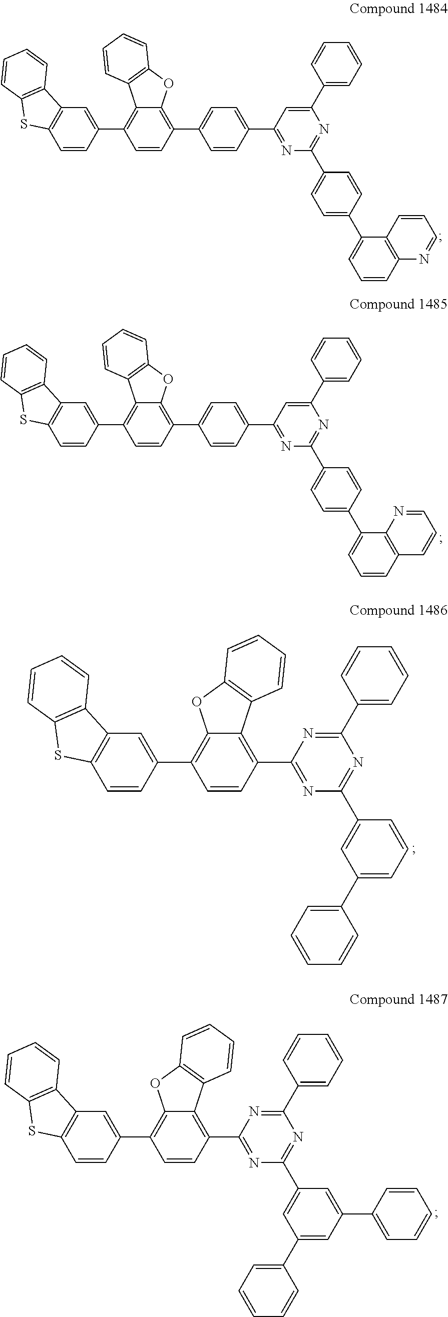 [SMILES: c1ccc(-c2cc(-c3ccc(-c4ccc(-c5ccc6sc7ccccc7c6c5)c5c4oc4ccccc45)cc3)nc(-c3ccc(-c4cccc5ncccc45)cc3)n2)cc1 ; c1ccc(-c2cc(-c3ccc(-c4ccc(-c5ccc6sc7ccccc7c6c5)c5c4oc4ccccc45)cc3)nc(-c3ccc(-c4cccc5cccnc45)cc3)n2)cc1 ; c1ccc(-c2cccc(-c3nc(-c4ccccc4)nc(-c4ccc(-c5ccc6sc7ccccc7c6c5)c5oc6ccccc6c45)n3)c2)cc1 ; c1ccc(-c2cc(-c3ccccc3)cc(-c3nc(-c4ccccc4)nc(-c4ccc(-c5ccc6sc7ccccc7c6c5)c5oc6ccccc6c45)n3)c2)cc1]

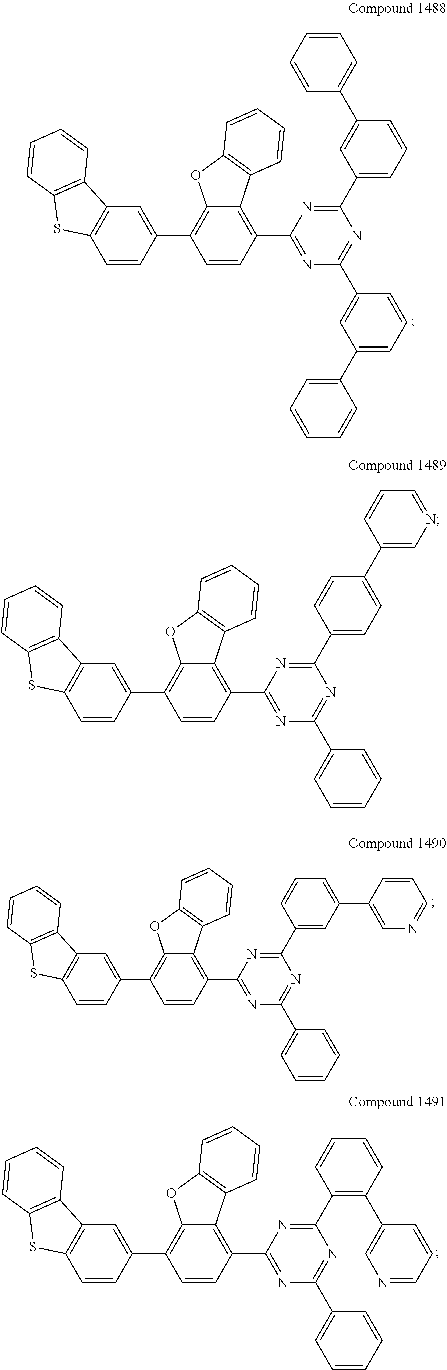 [SMILES: c1ccc(-c2cccc(-c3nc(-c4cccc(-c5ccccc5)c4)nc(-c4ccc(-c5ccc6sc7ccccc7c6c5)c5oc6ccccc6c45)n3)c2)cc1 ; c1ccc(-c2nc(-c3ccc(-c4cccnc4)cc3)nc(-c3ccc(-c4ccc5sc6ccccc6c5c4)c4oc5ccccc5c34)n2)cc1 ; c1ccc(-c2nc(-c3cccc(-c4cccnc4)c3)nc(-c3ccc(-c4ccc5sc6ccccc6c5c4)c4oc5ccccc5c34)n2)cc1 ; c1ccc(-c2nc(-c3ccccc3-c3cccnc3)nc(-c3ccc(-c4ccc5sc6ccccc6c5c4)c4oc5ccccc5c34)n2)cc1]

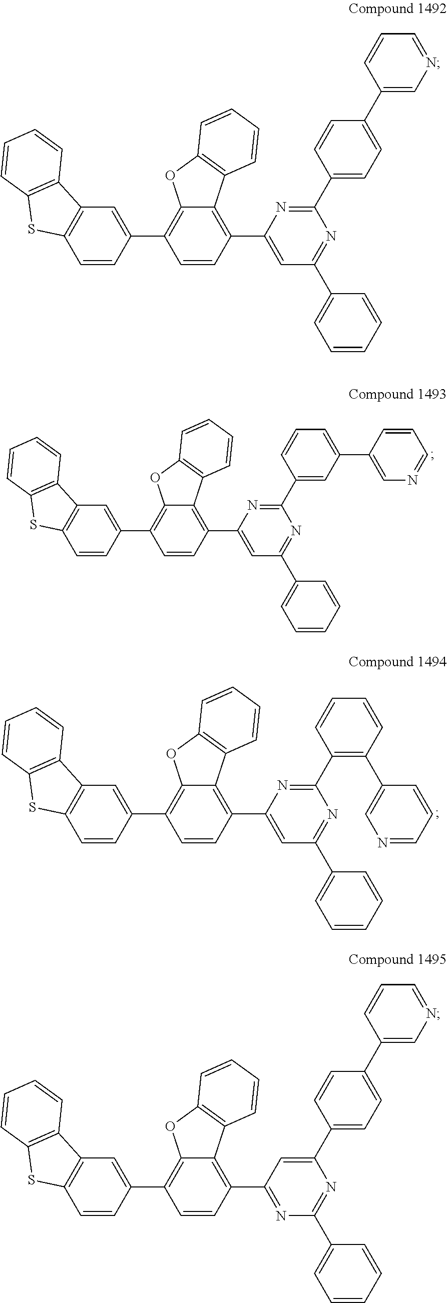 [SMILES: c1ccc(-c2cc(-c3ccc(-c4ccc5sc6ccccc6c5c4)c4oc5ccccc5c34)nc(-c3ccc(-c4cccnc4)cc3)n2)cc1 ; c1ccc(-c2cc(-c3ccc(-c4ccc5sc6ccccc6c5c4)c4oc5ccccc5c34)nc(-c3cccc(-c4cccnc4)c3)n2)cc1 ; c1ccc(-c2cc(-c3ccc(-c4ccc5sc6ccccc6c5c4)c4oc5ccccc5c34)nc(-c3ccccc3-c3cccnc3)n2)cc1 ; c1ccc(-c2nc(-c3ccc(-c4cccnc4)cc3)cc(-c3ccc(-c4ccc5sc6ccccc6c5c4)c4oc5ccccc5c34)n2)cc1]

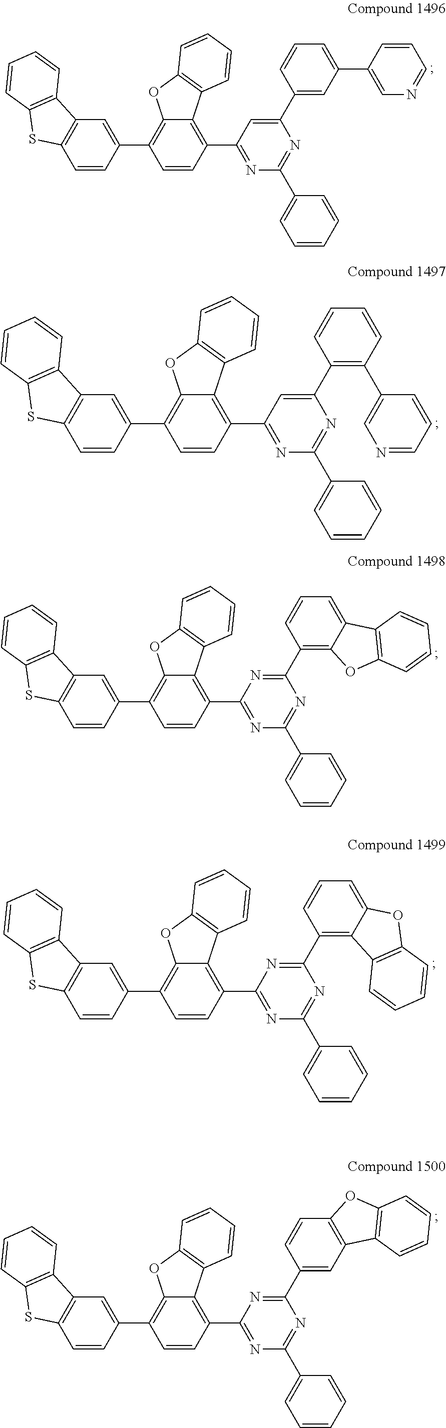 [SMILES: c1ccc(-c2nc(-c3cccc(-c4cccnc4)c3)cc(-c3ccc(-c4ccc5sc6ccccc6c5c4)c4oc5ccccc5c34)n2)cc1 ; c1ccc(-c2nc(-c3ccccc3-c3cccnc3)cc(-c3ccc(-c4ccc5sc6ccccc6c5c4)c4oc5ccccc5c34)n2)cc1 ; c1ccc(-c2nc(-c3cccc4c3oc3ccccc34)nc(-c3ccc(-c4ccc5sc6ccccc6c5c4)c4oc5ccccc5c34)n2)cc1 ; c1ccc(-c2nc(-c3cccc4oc5ccccc5c34)nc(-c3ccc(-c4ccc5sc6ccccc6c5c4)c4oc5ccccc5c34)n2)cc1 ; c1ccc(-c2nc(-c3ccc4oc5ccccc5c4c3)nc(-c3ccc(-c4ccc5sc6ccccc6c5c4)c4oc5ccccc5c34)n2)cc1]

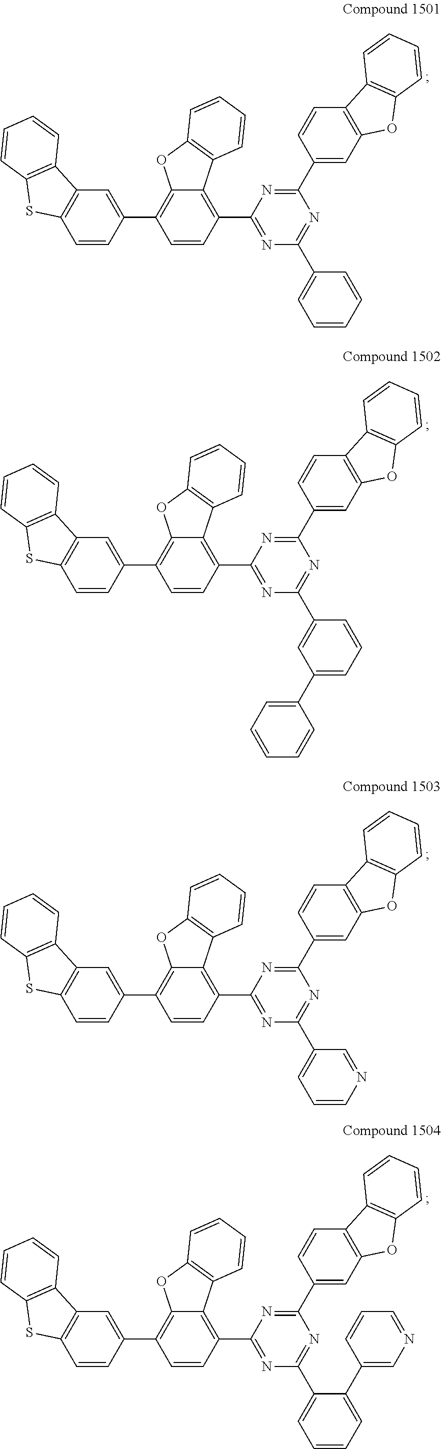 [SMILES: c1ccc(-c2nc(-c3ccc4c(c3)oc3ccccc34)nc(-c3ccc(-c4ccc5sc6ccccc6c5c4)c4oc5ccccc5c34)n2)cc1 ; c1ccc(-c2cccc(-c3nc(-c4ccc5c(c4)oc4ccccc45)nc(-c4ccc(-c5ccc6sc7ccccc7c6c5)c5oc6ccccc6c45)n3)c2)cc1 ; c1cncc(-c2nc(-c3ccc4c(c3)oc3ccccc34)nc(-c3ccc(-c4ccc5sc6ccccc6c5c4)c4oc5ccccc5c34)n2)c1 ; c1cncc(-c2ccccc2-c2nc(-c3ccc4c(c3)oc3ccccc34)nc(-c3ccc(-c4ccc5sc6ccccc6c5c4)c4oc5ccccc5c34)n2)c1]

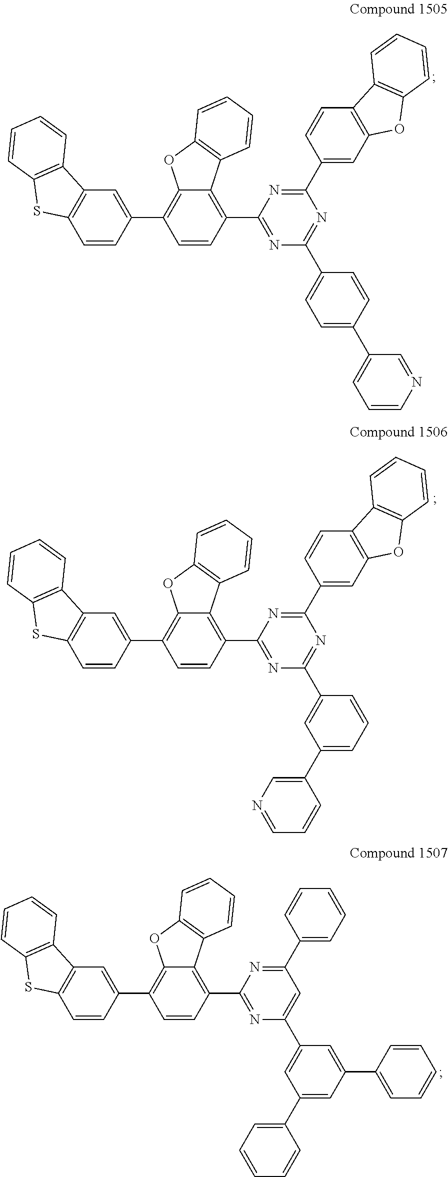 [SMILES: c1cncc(-c2ccc(-c3nc(-c4ccc5c(c4)oc4ccccc45)nc(-c4ccc(-c5ccc6sc7ccccc7c6c5)c5oc6ccccc6c45)n3)cc2)c1 ; c1cncc(-c2cccc(-c3nc(-c4ccc5c(c4)oc4ccccc45)nc(-c4ccc(-c5ccc6sc7ccccc7c6c5)c5oc6ccccc6c45)n3)c2)c1 ; c1ccc(-c2cc(-c3ccccc3)cc(-c3cc(-c4ccccc4)nc(-c4ccc(-c5ccc6sc7ccccc7c6c5)c5oc6ccccc6c45)n3)c2)cc1]

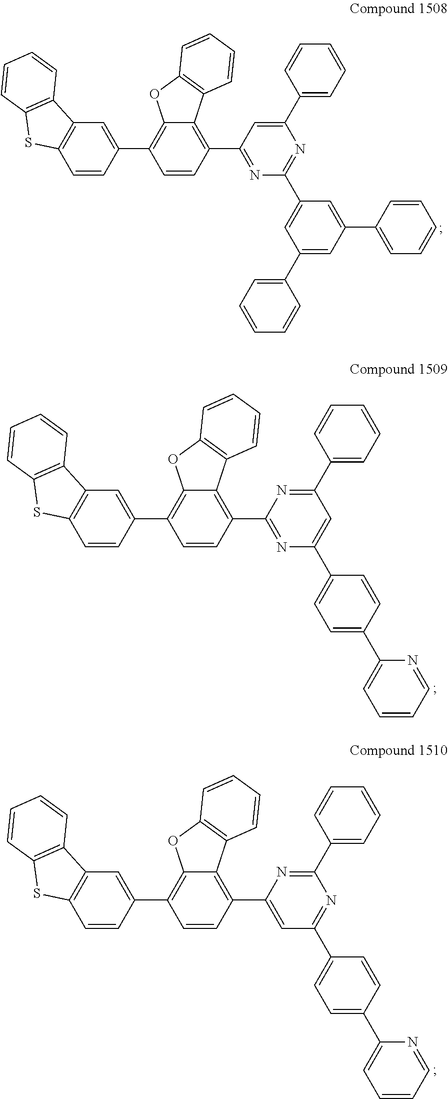 [SMILES: c1ccc(-c2cc(-c3ccccc3)cc(-c3nc(-c4ccccc4)cc(-c4ccc(-c5ccc6sc7ccccc7c6c5)c5oc6ccccc6c45)n3)c2)cc1 ; c1ccc(-c2cc(-c3ccc(-c4ccccn4)cc3)nc(-c3ccc(-c4ccc5sc6ccccc6c5c4)c4oc5ccccc5c34)n2)cc1 ; c1ccc(-c2nc(-c3ccc(-c4ccccn4)cc3)cc(-c3ccc(-c4ccc5sc6ccccc6c5c4)c4oc5ccccc5c34)n2)cc1]

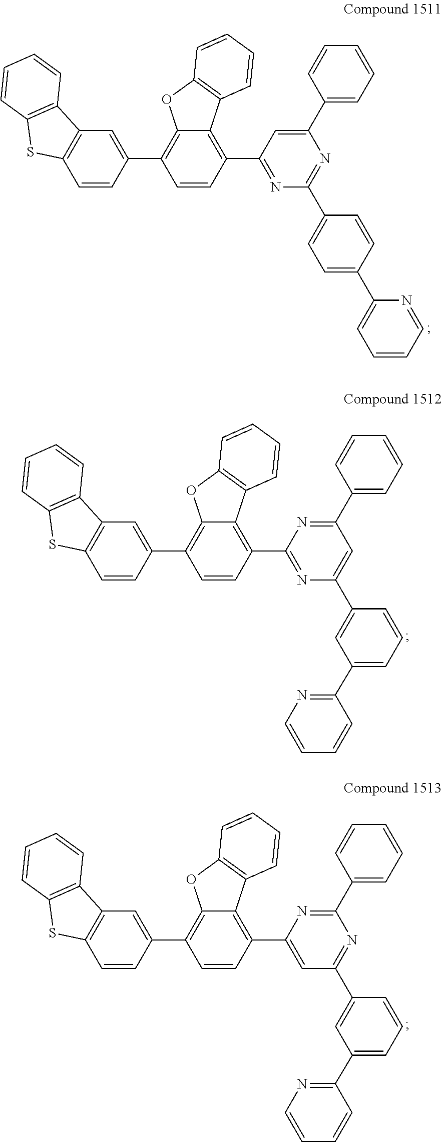 [SMILES: c1ccc(-c2cc(-c3ccc(-c4ccc5sc6ccccc6c5c4)c4oc5ccccc5c34)nc(-c3ccc(-c4ccccn4)cc3)n2)cc1 ; c1ccc(-c2cc(-c3cccc(-c4ccccn4)c3)nc(-c3ccc(-c4ccc5sc6ccccc6c5c4)c4oc5ccccc5c34)n2)cc1 ; c1ccc(-c2nc(-c3cccc(-c4ccccn4)c3)cc(-c3ccc(-c4ccc5sc6ccccc6c5c4)c4oc5ccccc5c34)n2)cc1]

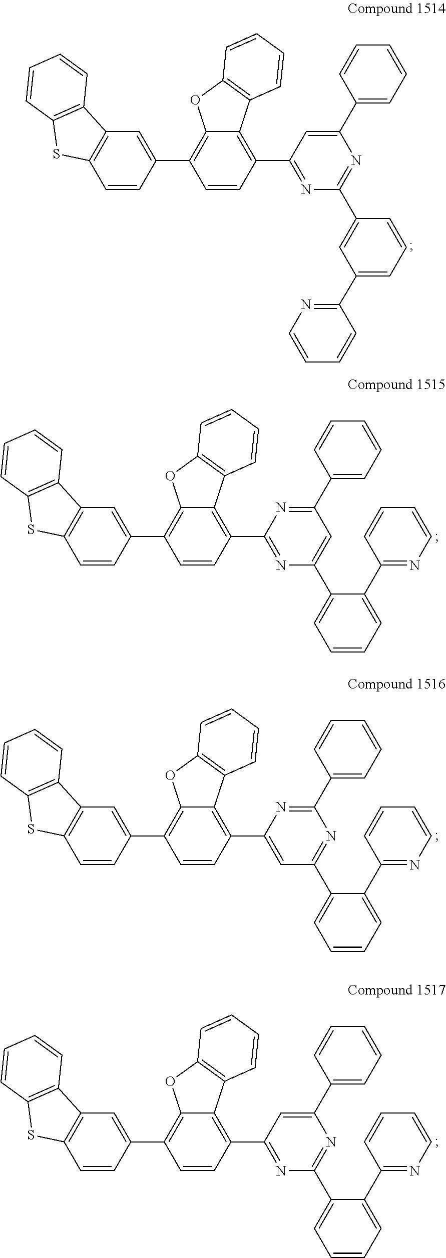 [SMILES: c1ccc(-c2cc(-c3ccc(-c4ccc5sc6ccccc6c5c4)c4oc5ccccc5c34)nc(-c3cccc(-c4ccccn4)c3)n2)cc1 ; c1ccc(-c2cc(-c3ccccc3-c3ccccn3)nc(-c3ccc(-c4ccc5sc6ccccc6c5c4)c4oc5ccccc5c34)n2)cc1 ; c1ccc(-c2nc(-c3ccccc3-c3ccccn3)cc(-c3ccc(-c4ccc5sc6ccccc6c5c4)c4oc5ccccc5c34)n2)cc1 ; c1ccc(-c2cc(-c3ccc(-c4ccc5sc6ccccc6c5c4)c4oc5ccccc5c34)nc(-c3ccccc3-c3ccccn3)n2)cc1]

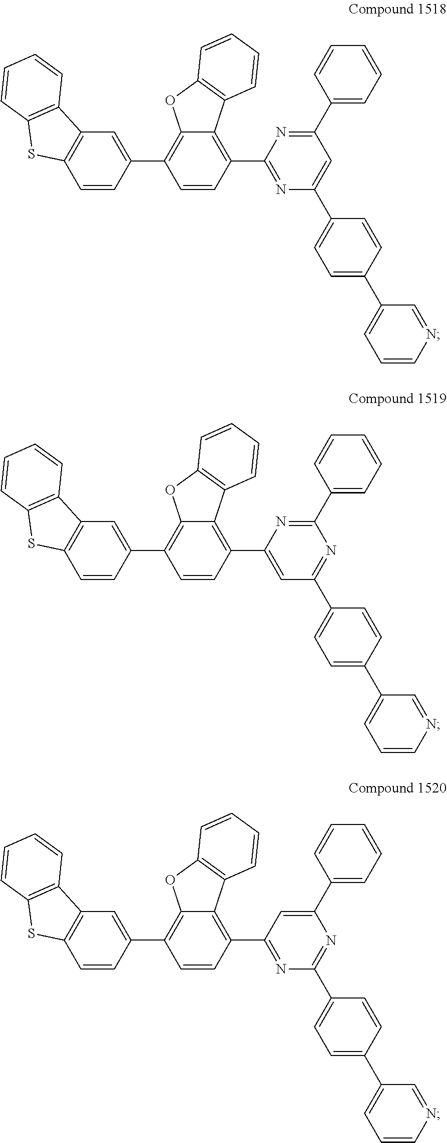 [SMILES: c1ccc(-c2cc(-c3ccc(-c4cccnc4)cc3)nc(-c3ccc(-c4ccc5sc6ccccc6c5c4)c4oc5ccccc5c34)n2)cc1 ; c1ccc(-c2nc(-c3ccc(-c4cccnc4)cc3)cc(-c3ccc(-c4ccc5sc6ccccc6c5c4)c4oc5ccccc5c34)n2)cc1 ; c1ccc(-c2cc(-c3ccc(-c4ccc5sc6ccccc6c5c4)c4oc5ccccc5c34)nc(-c3ccc(-c4cccnc4)cc3)n2)cc1]

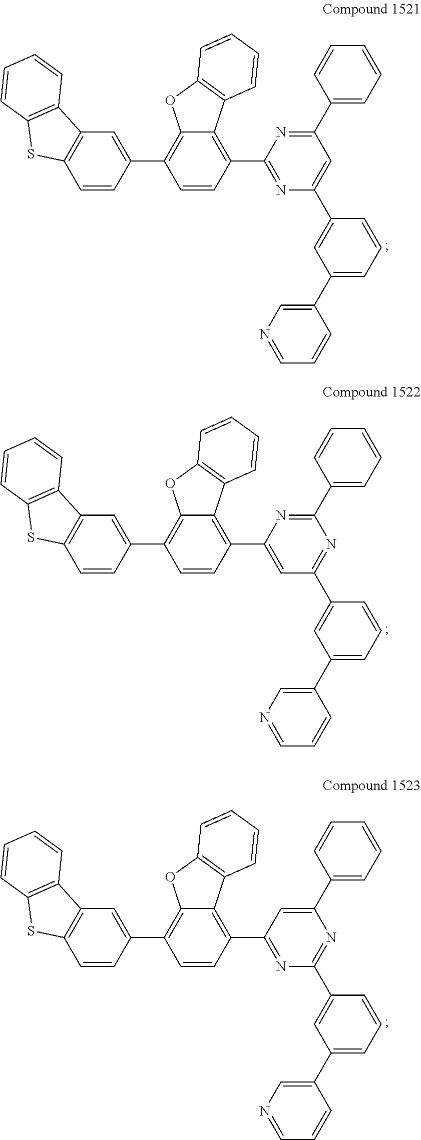 [SMILES: c1ccc(-c2cc(-c3cccc(-c4cccnc4)c3)nc(-c3ccc(-c4ccc5sc6ccccc6c5c4)c4oc5ccccc5c34)n2)cc1 ; c1ccc(-c2nc(-c3cccc(-c4cccnc4)c3)cc(-c3ccc(-c4ccc5sc6ccccc6c5c4)c4oc5ccccc5c34)n2)cc1 ; c1ccc(-c2cc(-c3ccc(-c4ccc5sc6ccccc6c5c4)c4oc5ccccc5c34)nc(-c3cccc(-c4cccnc4)c3)n2)cc1]

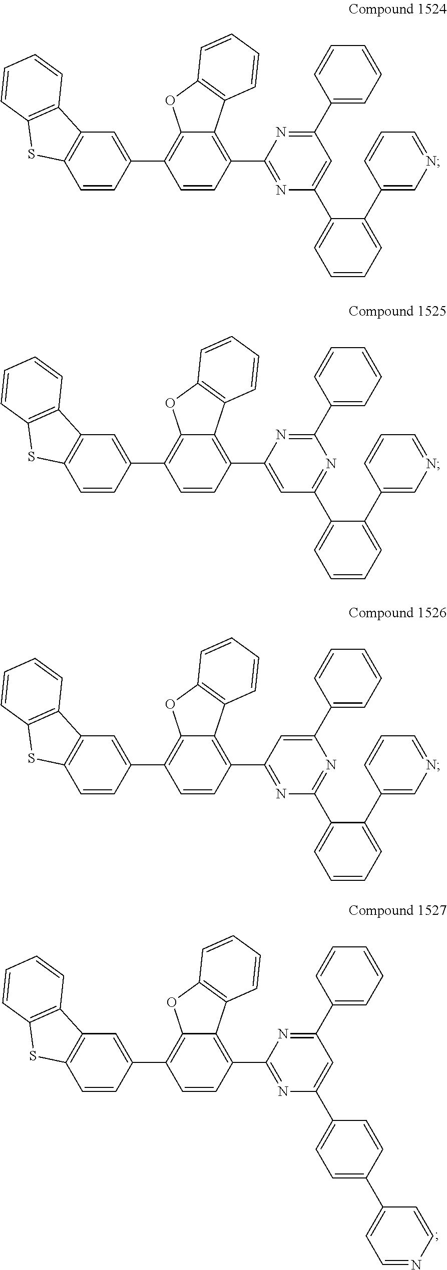 [SMILES: c1ccc(-c2cc(-c3ccccc3-c3cccnc3)nc(-c3ccc(-c4ccc5sc6ccccc6c5c4)c4oc5ccccc5c34)n2)cc1 ; c1ccc(-c2nc(-c3ccccc3-c3cccnc3)cc(-c3ccc(-c4ccc5sc6ccccc6c5c4)c4oc5ccccc5c34)n2)cc1 ; c1ccc(-c2cc(-c3ccc(-c4ccc5sc6ccccc6c5c4)c4oc5ccccc5c34)nc(-c3ccccc3-c3cccnc3)n2)cc1 ; c1ccc(-c2cc(-c3ccc(-c4ccncc4)cc3)nc(-c3ccc(-c4ccc5sc6ccccc6c5c4)c4oc5ccccc5c34)n2)cc1]

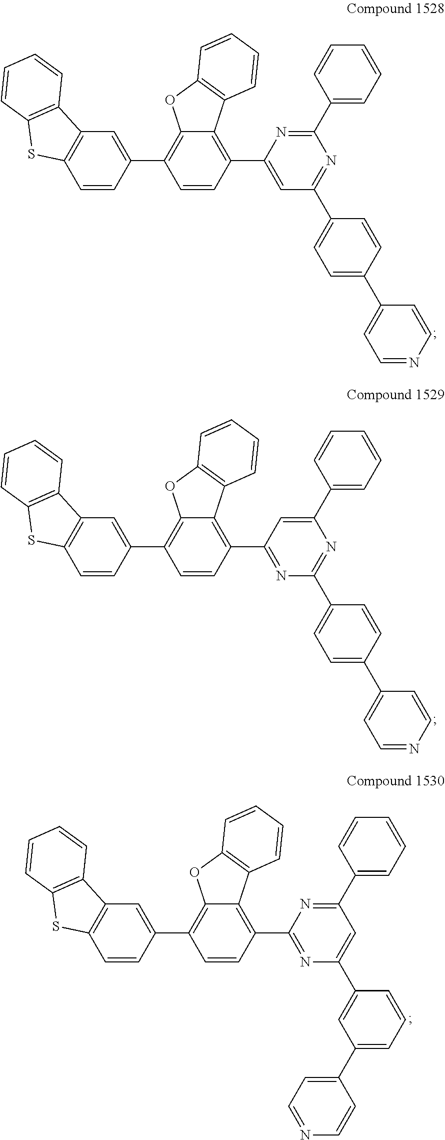 [SMILES: c1ccc(-c2nc(-c3ccc(-c4ccncc4)cc3)cc(-c3ccc(-c4ccc5sc6ccccc6c5c4)c4oc5ccccc5c34)n2)cc1 ; c1ccc(-c2cc(-c3ccc(-c4ccc5sc6ccccc6c5c4)c4oc5ccccc5c34)nc(-c3ccc(-c4ccncc4)cc3)n2)cc1 ; c1ccc(-c2cc(-c3cccc(-c4ccncc4)c3)nc(-c3ccc(-c4ccc5sc6ccccc6c5c4)c4oc5ccccc5c34)n2)cc1]

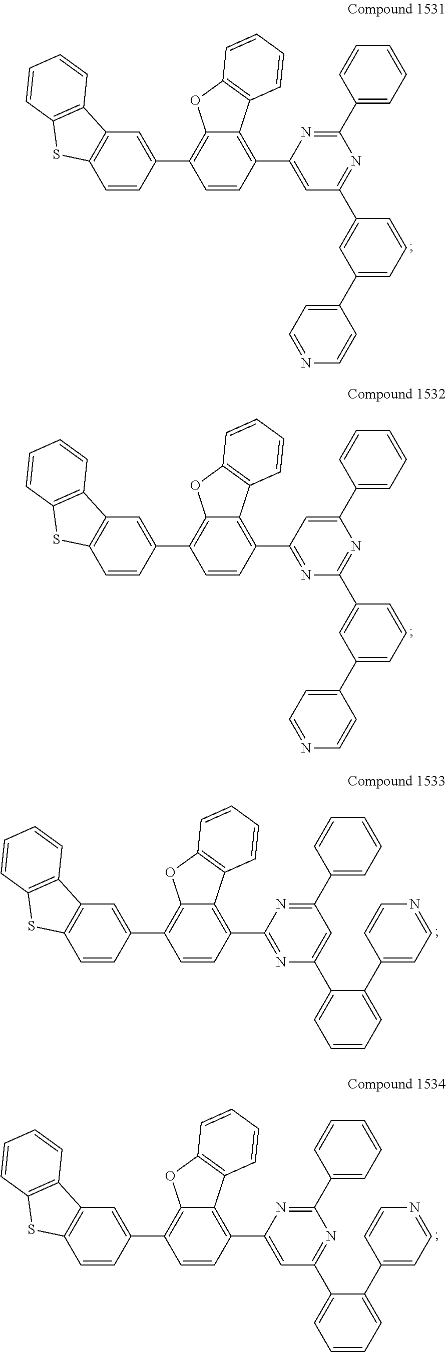 [SMILES: c1ccc(-c2nc(-c3cccc(-c4ccncc4)c3)cc(-c3ccc(-c4ccc5sc6ccccc6c5c4)c4oc5ccccc5c34)n2)cc1 ; c1ccc(-c2cc(-c3ccc(-c4ccc5sc6ccccc6c5c4)c4oc5ccccc5c34)nc(-c3cccc(-c4ccncc4)c3)n2)cc1 ; c1ccc(-c2cc(-c3ccccc3-c3ccncc3)nc(-c3ccc(-c4ccc5sc6ccccc6c5c4)c4oc5ccccc5c34)n2)cc1 ; c1ccc(-c2nc(-c3ccccc3-c3ccncc3)cc(-c3ccc(-c4ccc5sc6ccccc6c5c4)c4oc5ccccc5c34)n2)cc1]

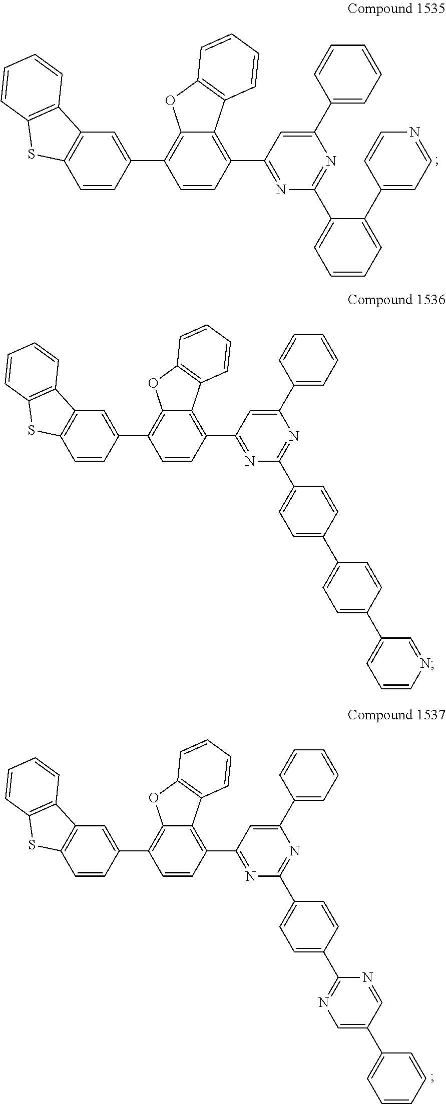 [SMILES: c1ccc(-c2cc(-c3ccc(-c4ccc5sc6ccccc6c5c4)c4oc5ccccc5c34)nc(-c3ccccc3-c3ccncc3)n2)cc1 ; c1ccc(-c2cc(-c3ccc(-c4ccc5sc6ccccc6c5c4)c4oc5ccccc5c34)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc7sc8ccccc8c7c6)c6oc7ccccc7c56)n4)cc3)nc2)cc1]

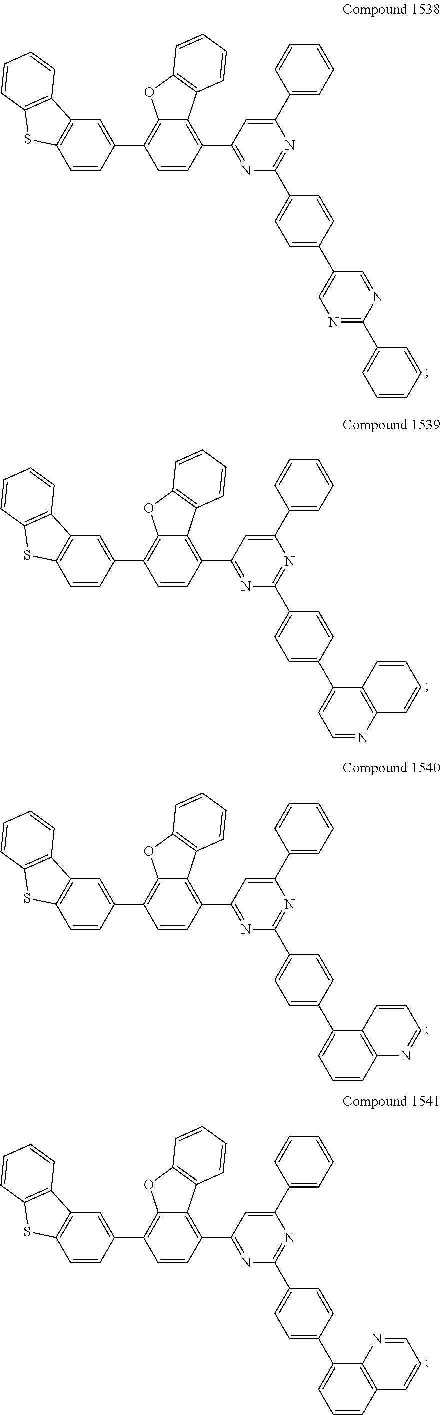 [SMILES: c1ccc(-c2cc(-c3ccc(-c4ccc5sc6ccccc6c5c4)c4oc5ccccc5c34)nc(-c3ccc(-c4cnc(-c5ccccc5)nc4)cc3)n2)cc1 ; c1ccc(-c2cc(-c3ccc(-c4ccc5sc6ccccc6c5c4)c4oc5ccccc5c34)nc(-c3ccc(-c4ccnc5ccccc45)cc3)n2)cc1 ; c1ccc(-c2cc(-c3ccc(-c4ccc5sc6ccccc6c5c4)c4oc5ccccc5c34)nc(-c3ccc(-c4cccc5ncccc45)cc3)n2)cc1 ; c1ccc(-c2cc(-c3ccc(-c4ccc5sc6ccccc6c5c4)c4oc5ccccc5c34)nc(-c3ccc(-c4cccc5cccnc45)cc3)n2)cc1]

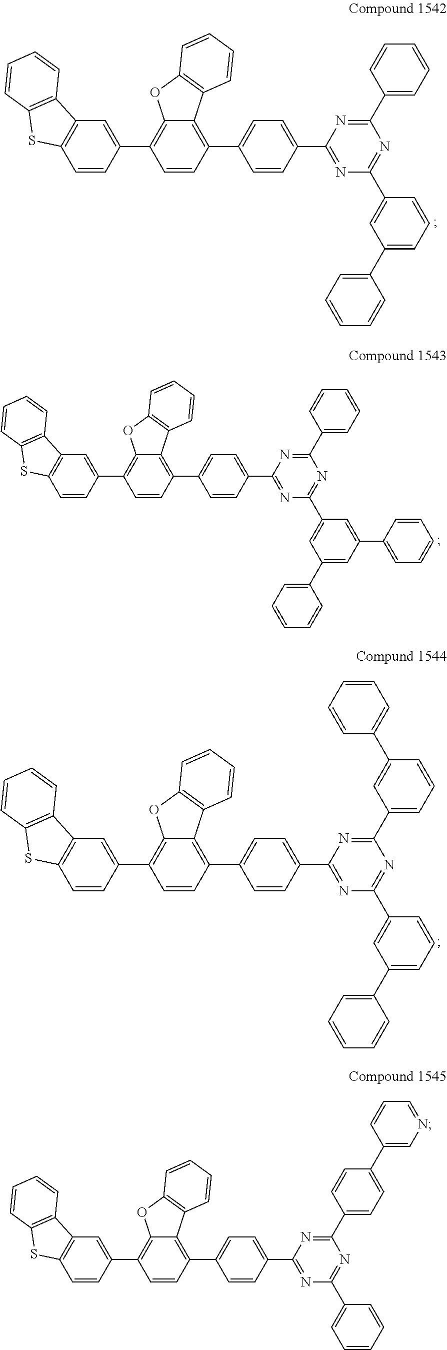 [SMILES: c1ccc(-c2cccc(-c3nc(-c4ccccc4)nc(-c4ccc(-c5ccc(-c6ccc7sc8ccccc8c7c6)c6oc7ccccc7c56)cc4)n3)c2)cc1 ; c1ccc(-c2cc(-c3ccccc3)cc(-c3nc(-c4ccccc4)nc(-c4ccc(-c5ccc(-c6ccc7sc8ccccc8c7c6)c6oc7ccccc7c56)cc4)n3)c2)cc1 ; c1ccc(-c2cccc(-c3nc(-c4ccc(-c5ccc(-c6ccc7sc8ccccc8c7c6)c6oc7ccccc7c56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5ccc6sc7ccccc7c6c5)c5oc6ccccc6c45)cc3)n2)cc1]

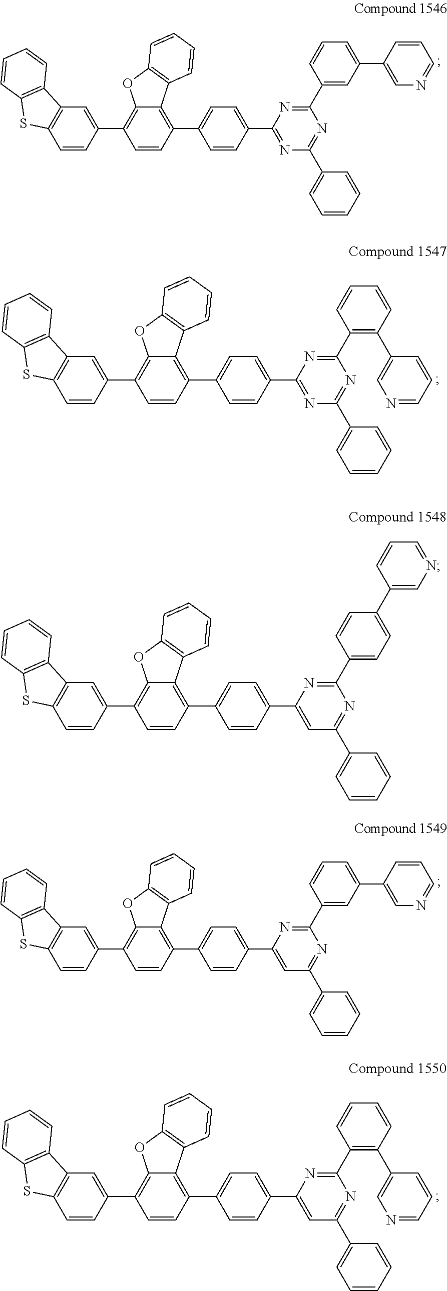 [SMILES: c1ccc(-c2nc(-c3ccc(-c4ccc(-c5ccc6sc7ccccc7c6c5)c5oc6ccccc6c45)cc3)nc(-c3cccc(-c4cccnc4)c3)n2)cc1 ; c1ccc(-c2nc(-c3ccc(-c4ccc(-c5ccc6sc7ccccc7c6c5)c5oc6ccccc6c45)cc3)nc(-c3ccccc3-c3cccnc3)n2)cc1 ; c1ccc(-c2cc(-c3ccc(-c4ccc(-c5ccc6sc7ccccc7c6c5)c5oc6ccccc6c45)cc3)nc(-c3ccc(-c4cccnc4)cc3)n2)cc1 ; c1ccc(-c2cc(-c3ccc(-c4ccc(-c5ccc6sc7ccccc7c6c5)c5oc6ccccc6c45)cc3)nc(-c3cccc(-c4cccnc4)c3)n2)cc1 ; c1ccc(-c2cc(-c3ccc(-c4ccc(-c5ccc6sc7ccccc7c6c5)c5oc6ccccc6c45)cc3)nc(-c3ccccc3-c3cccnc3)n2)cc1]

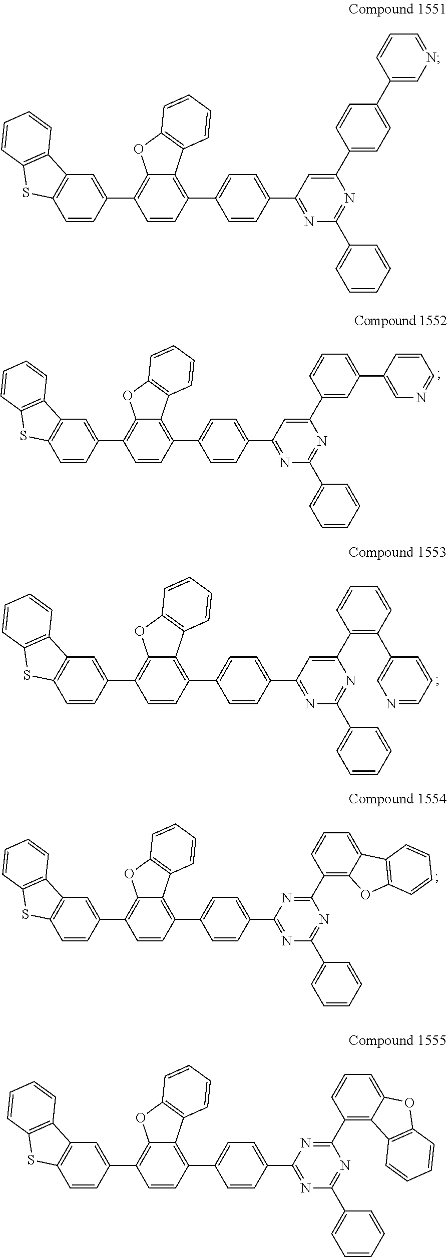 [SMILES: c1ccc(-c2nc(-c3ccc(-c4cccnc4)cc3)cc(-c3ccc(-c4ccc(-c5ccc6sc7ccccc7c6c5)c5oc6ccccc6c45)cc3)n2)cc1 ; c1ccc(-c2nc(-c3ccc(-c4ccc(-c5ccc6sc7ccccc7c6c5)c5oc6ccccc6c45)cc3)cc(-c3cccc(-c4cccnc4)c3)n2)cc1 ; c1ccc(-c2nc(-c3ccc(-c4ccc(-c5ccc6sc7ccccc7c6c5)c5oc6ccccc6c45)cc3)cc(-c3ccccc3-c3cccnc3)n2)cc1 ; c1ccc(-c2nc(-c3ccc(-c4ccc(-c5ccc6sc7ccccc7c6c5)c5oc6ccccc6c45)cc3)nc(-c3cccc4c3oc3ccccc34)n2)cc1 ; c1ccc(-c2nc(-c3ccc(-c4ccc(-c5ccc6sc7ccccc7c6c5)c5oc6ccccc6c45)cc3)nc(-c3cccc4oc5ccccc5c34)n2)cc1]

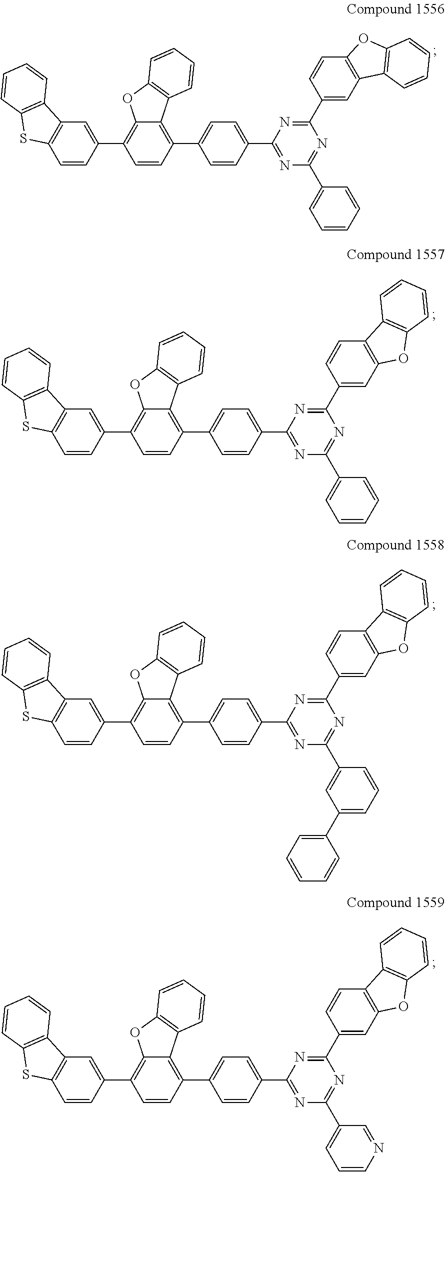 [SMILES: c1ccc(-c2nc(-c3ccc(-c4ccc(-c5ccc6sc7ccccc7c6c5)c5oc6ccccc6c45)cc3)nc(-c3ccc4oc5ccccc5c4c3)n2)cc1 ; c1ccc(-c2nc(-c3ccc(-c4ccc(-c5ccc6sc7ccccc7c6c5)c5oc6ccccc6c45)cc3)nc(-c3ccc4c(c3)oc3ccccc34)n2)cc1 ; c1ccc(-c2cccc(-c3nc(-c4ccc(-c5ccc(-c6ccc7sc8ccccc8c7c6)c6oc7ccccc7c56)cc4)nc(-c4ccc5c(c4)oc4ccccc45)n3)c2)cc1 ; c1cncc(-c2nc(-c3ccc(-c4ccc(-c5ccc6sc7ccccc7c6c5)c5oc6ccccc6c45)cc3)nc(-c3ccc4c(c3)oc3ccccc34)n2)c1]

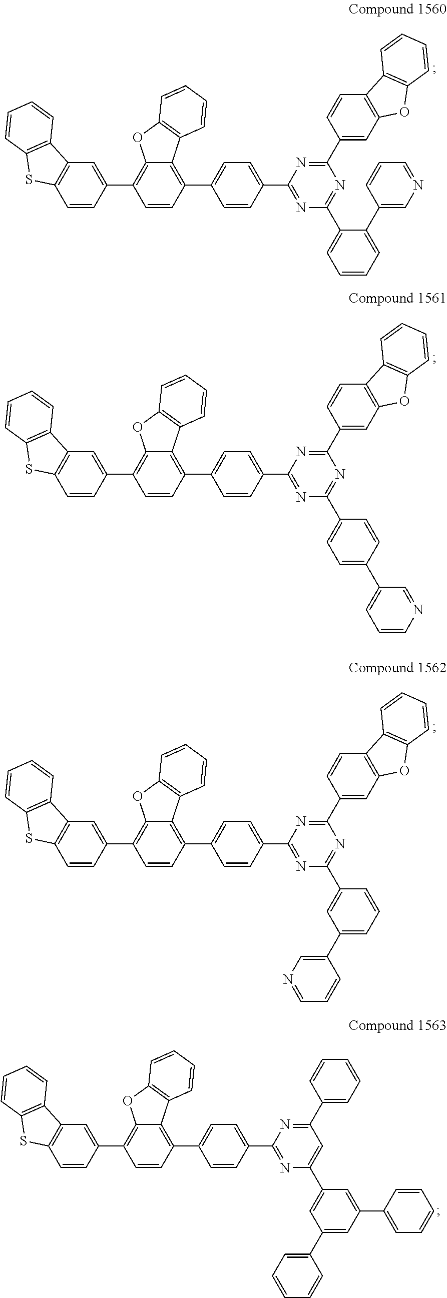 [SMILES: c1cncc(-c2ccccc2-c2nc(-c3ccc(-c4ccc(-c5ccc6sc7ccccc7c6c5)c5oc6ccccc6c45)cc3)nc(-c3ccc4c(c3)oc3ccccc34)n2)c1 ; c1cncc(-c2ccc(-c3nc(-c4ccc(-c5ccc(-c6ccc7sc8ccccc8c7c6)c6oc7ccccc7c56)cc4)nc(-c4ccc5c(c4)oc4ccccc45)n3)cc2)c1 ; c1cncc(-c2cccc(-c3nc(-c4ccc(-c5ccc(-c6ccc7sc8ccccc8c7c6)c6oc7ccccc7c56)cc4)nc(-c4ccc5c(c4)oc4ccccc45)n3)c2)c1 ; c1ccc(-c2cc(-c3ccccc3)cc(-c3cc(-c4ccccc4)nc(-c4ccc(-c5ccc(-c6ccc7sc8ccccc8c7c6)c6oc7ccccc7c56)cc4)n3)c2)cc1]

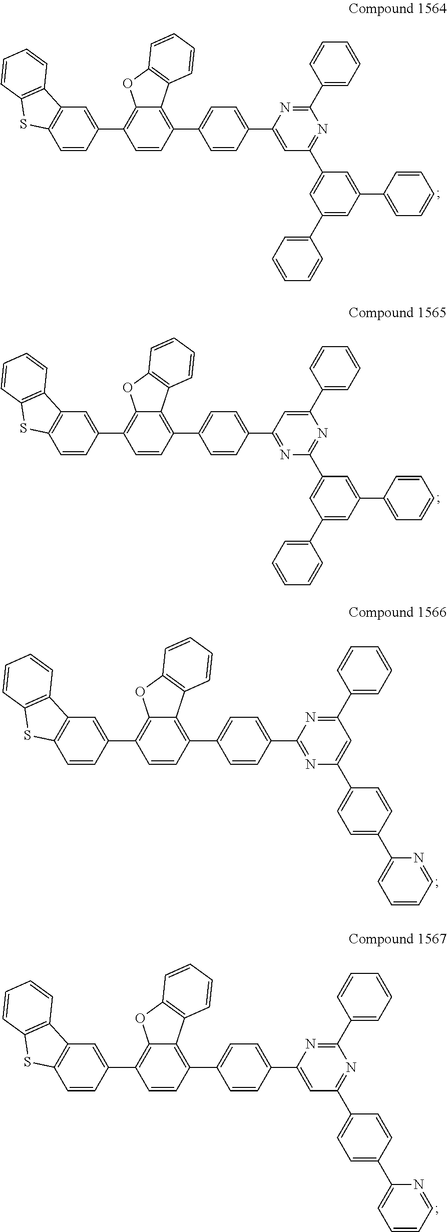 [SMILES: c1ccc(-c2cc(-c3ccccc3)cc(-c3cc(-c4ccc(-c5ccc(-c6ccc7sc8ccccc8c7c6)c6oc7ccccc7c56)cc4)nc(-c4ccccc4)n3)c2)cc1 ; c1ccc(-c2cc(-c3ccccc3)cc(-c3nc(-c4ccccc4)cc(-c4ccc(-c5ccc(-c6ccc7sc8ccccc8c7c6)c6oc7ccccc7c56)cc4)n3)c2)cc1 ; c1ccc(-c2cc(-c3ccc(-c4ccccn4)cc3)nc(-c3ccc(-c4ccc(-c5ccc6sc7ccccc7c6c5)c5oc6ccccc6c45)cc3)n2)cc1 ; c1ccc(-c2nc(-c3ccc(-c4ccccn4)cc3)cc(-c3ccc(-c4ccc(-c5ccc6sc7ccccc7c6c5)c5oc6ccccc6c45)cc3)n2)cc1]

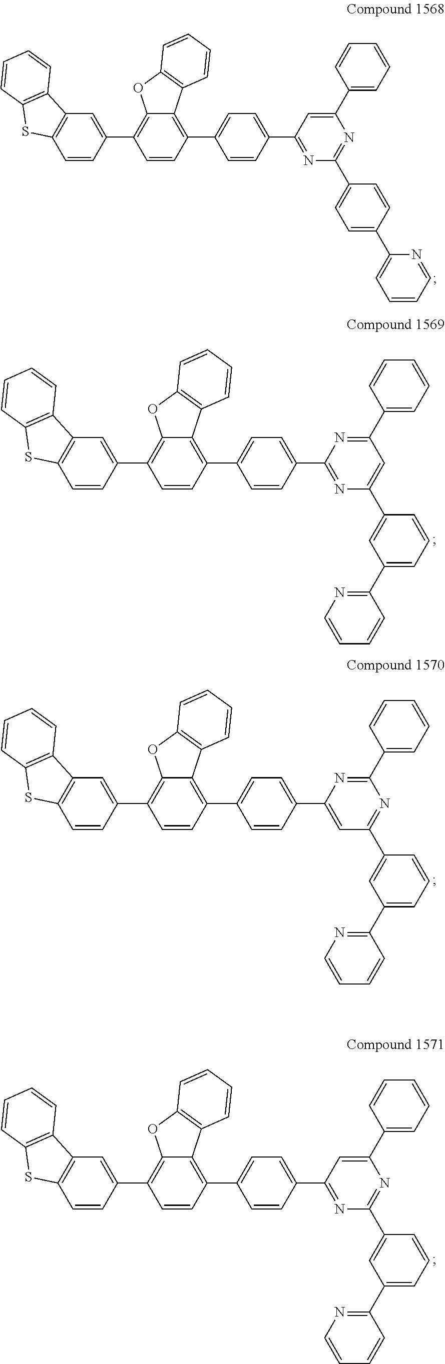 [SMILES: c1ccc(-c2cc(-c3ccc(-c4ccc(-c5ccc6sc7ccccc7c6c5)c5oc6ccccc6c45)cc3)nc(-c3ccc(-c4ccccn4)cc3)n2)cc1 ; c1ccc(-c2cc(-c3cccc(-c4ccccn4)c3)nc(-c3ccc(-c4ccc(-c5ccc6sc7ccccc7c6c5)c5oc6ccccc6c45)cc3)n2)cc1 ; c1ccc(-c2nc(-c3ccc(-c4ccc(-c5ccc6sc7ccccc7c6c5)c5oc6ccccc6c45)cc3)cc(-c3cccc(-c4ccccn4)c3)n2)cc1 ; c1ccc(-c2cc(-c3ccc(-c4ccc(-c5ccc6sc7ccccc7c6c5)c5oc6ccccc6c45)cc3)nc(-c3cccc(-c4ccccn4)c3)n2)cc1]

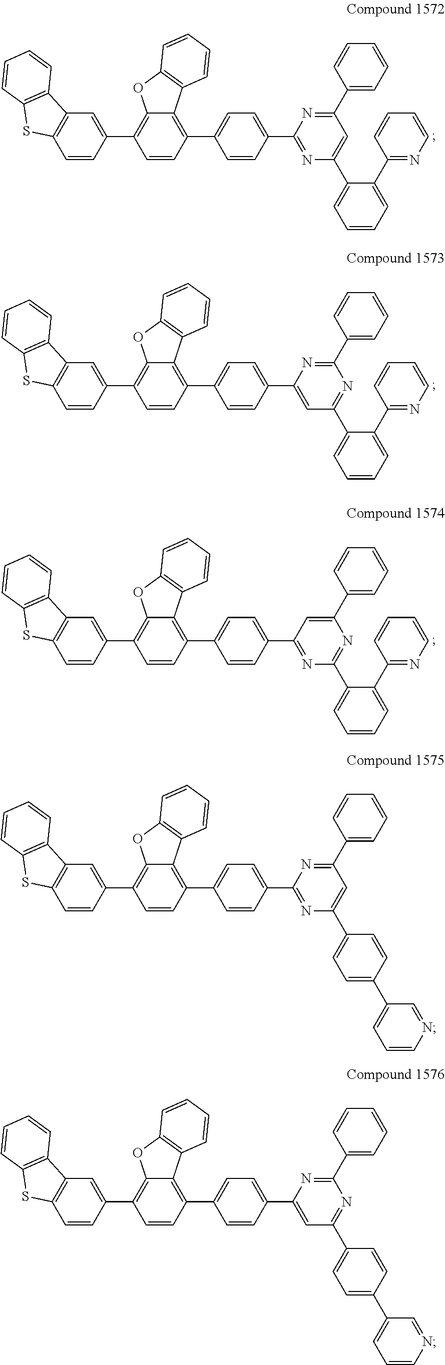 [SMILES: c1ccc(-c2cc(-c3ccccc3-c3ccccn3)nc(-c3ccc(-c4ccc(-c5ccc6sc7ccccc7c6c5)c5oc6ccccc6c45)cc3)n2)cc1 ; c1ccc(-c2nc(-c3ccc(-c4ccc(-c5ccc6sc7ccccc7c6c5)c5oc6ccccc6c45)cc3)cc(-c3ccccc3-c3ccccn3)n2)cc1 ; c1ccc(-c2cc(-c3ccc(-c4ccc(-c5ccc6sc7ccccc7c6c5)c5oc6ccccc6c45)cc3)nc(-c3ccccc3-c3ccccn3)n2)cc1 ; c1ccc(-c2cc(-c3ccc(-c4cccnc4)cc3)nc(-c3ccc(-c4ccc(-c5ccc6sc7ccccc7c6c5)c5oc6ccccc6c45)cc3)n2)cc1 ; c1ccc(-c2nc(-c3ccc(-c4cccnc4)cc3)cc(-c3ccc(-c4ccc(-c5ccc6sc7ccccc7c6c5)c5oc6ccccc6c45)cc3)n2)cc1]

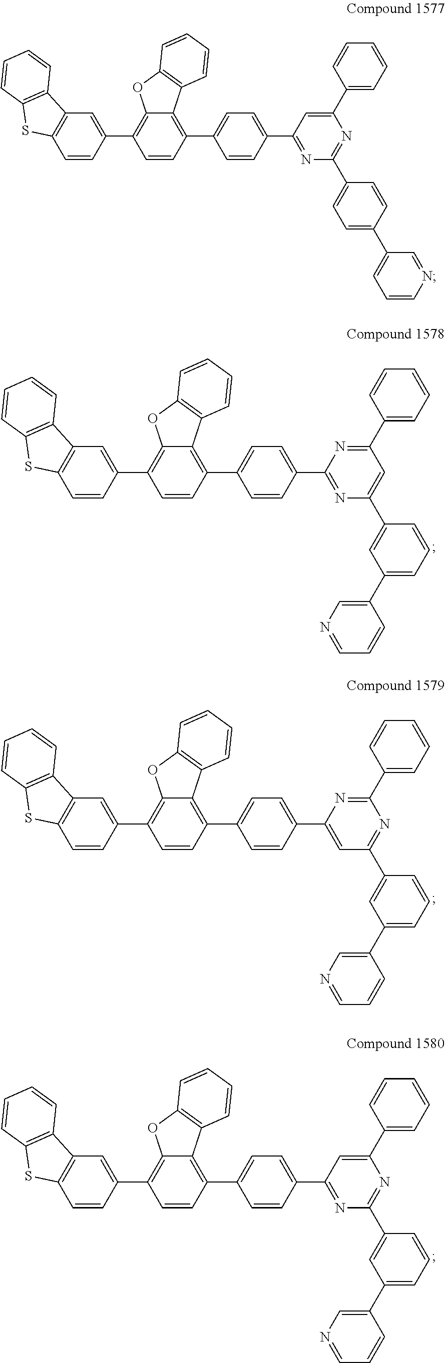 [SMILES: c1ccc(-c2cc(-c3ccc(-c4ccc(-c5ccc6sc7ccccc7c6c5)c5oc6ccccc6c45)cc3)nc(-c3ccc(-c4cccnc4)cc3)n2)cc1 ; c1ccc(-c2cc(-c3cccc(-c4cccnc4)c3)nc(-c3ccc(-c4ccc(-c5ccc6sc7ccccc7c6c5)c5oc6ccccc6c45)cc3)n2)cc1 ; c1ccc(-c2nc(-c3ccc(-c4ccc(-c5ccc6sc7ccccc7c6c5)c5oc6ccccc6c45)cc3)cc(-c3cccc(-c4cccnc4)c3)n2)cc1 ; c1ccc(-c2cc(-c3ccc(-c4ccc(-c5ccc6sc7ccccc7c6c5)c5oc6ccccc6c45)cc3)nc(-c3cccc(-c4cccnc4)c3)n2)cc1]

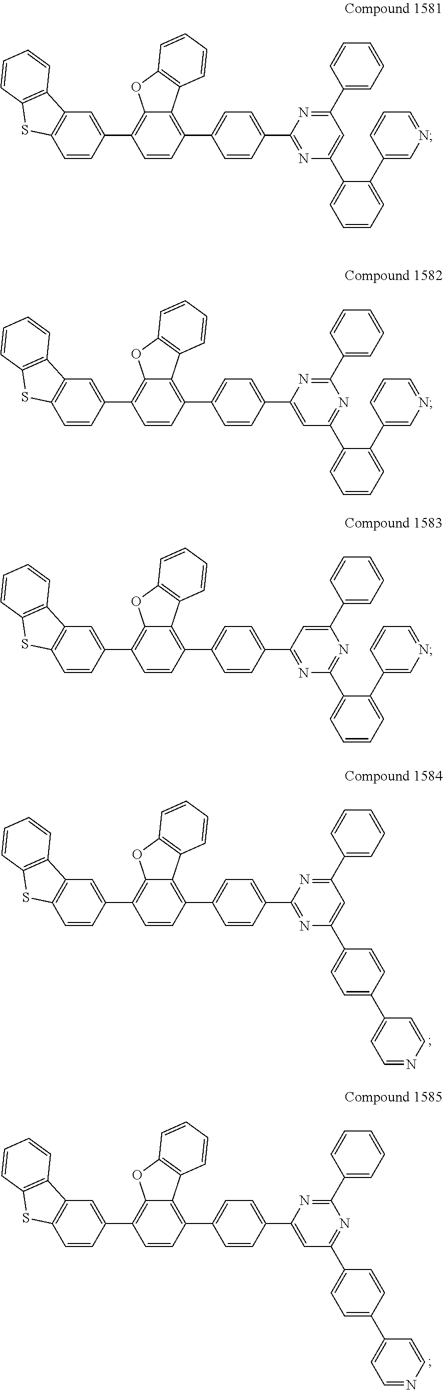 [SMILES: c1ccc(-c2cc(-c3ccccc3-c3cccnc3)nc(-c3ccc(-c4ccc(-c5ccc6sc7ccccc7c6c5)c5oc6ccccc6c45)cc3)n2)cc1 ; c1ccc(-c2nc(-c3ccc(-c4ccc(-c5ccc6sc7ccccc7c6c5)c5oc6ccccc6c45)cc3)cc(-c3ccccc3-c3cccnc3)n2)cc1 ; c1ccc(-c2cc(-c3ccc(-c4ccc(-c5ccc6sc7ccccc7c6c5)c5oc6ccccc6c45)cc3)nc(-c3ccccc3-c3cccnc3)n2)cc1 ; c1ccc(-c2cc(-c3ccc(-c4ccncc4)cc3)nc(-c3ccc(-c4ccc(-c5ccc6sc7ccccc7c6c5)c5oc6ccccc6c45)cc3)n2)cc1 ; c1ccc(-c2nc(-c3ccc(-c4ccncc4)cc3)cc(-c3ccc(-c4ccc(-c5ccc6sc7ccccc7c6c5)c5oc6ccccc6c45)cc3)n2)cc1]

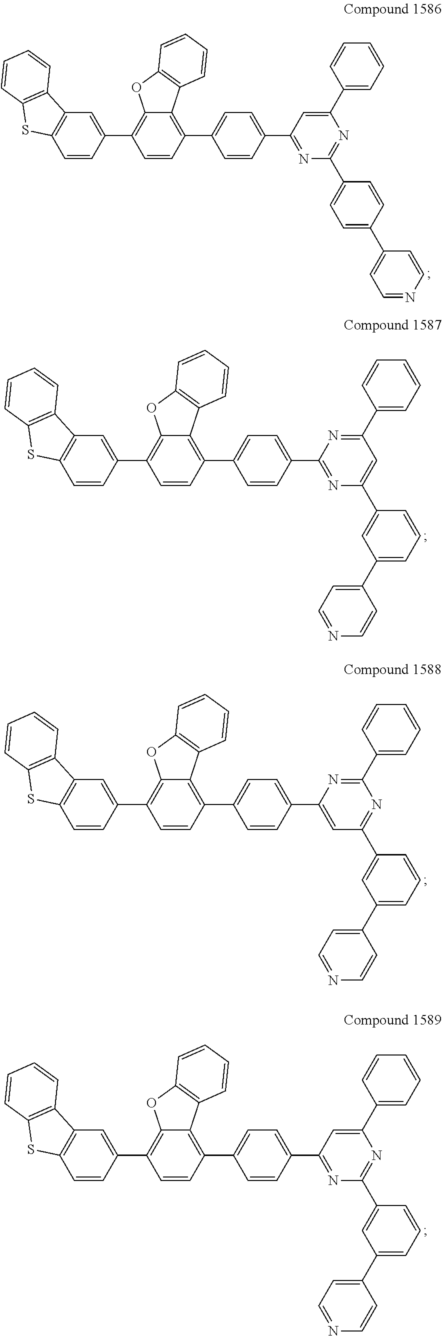 [SMILES: c1ccc(-c2cc(-c3ccc(-c4ccc(-c5ccc6sc7ccccc7c6c5)c5oc6ccccc6c45)cc3)nc(-c3ccc(-c4ccncc4)cc3)n2)cc1 ; c1ccc(-c2cc(-c3cccc(-c4ccncc4)c3)nc(-c3ccc(-c4ccc(-c5ccc6sc7ccccc7c6c5)c5oc6ccccc6c45)cc3)n2)cc1 ; c1ccc(-c2nc(-c3ccc(-c4ccc(-c5ccc6sc7ccccc7c6c5)c5oc6ccccc6c45)cc3)cc(-c3cccc(-c4ccncc4)c3)n2)cc1 ; c1ccc(-c2cc(-c3ccc(-c4ccc(-c5ccc6sc7ccccc7c6c5)c5oc6ccccc6c45)cc3)nc(-c3cccc(-c4ccncc4)c3)n2)cc1]

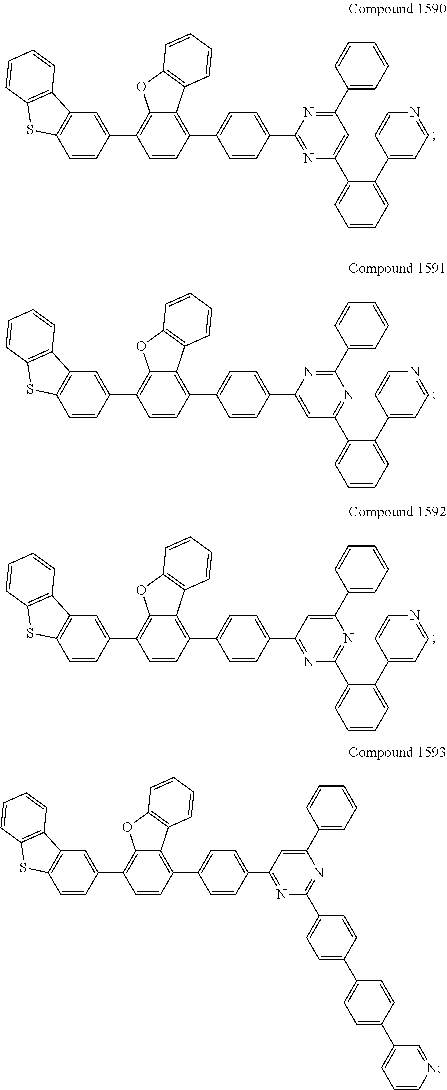 [SMILES: c1ccc(-c2cc(-c3ccccc3-c3ccncc3)nc(-c3ccc(-c4ccc(-c5ccc6sc7ccccc7c6c5)c5oc6ccccc6c45)cc3)n2)cc1 ; c1ccc(-c2nc(-c3ccc(-c4ccc(-c5ccc6sc7ccccc7c6c5)c5oc6ccccc6c45)cc3)cc(-c3ccccc3-c3ccncc3)n2)cc1 ; c1ccc(-c2cc(-c3ccc(-c4ccc(-c5ccc6sc7ccccc7c6c5)c5oc6ccccc6c45)cc3)nc(-c3ccccc3-c3ccncc3)n2)cc1 ; c1ccc(-c2cc(-c3ccc(-c4ccc(-c5ccc6sc7ccccc7c6c5)c5oc6ccccc6c45)cc3)nc(-c3ccc(-c4ccc(-c5cccnc5)cc4)cc3)n2)cc1]

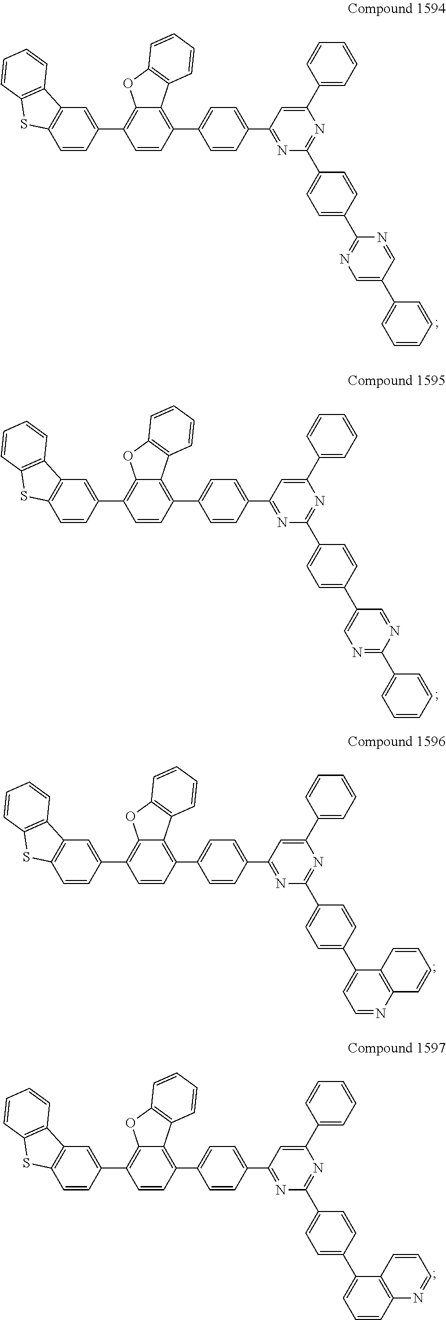 [SMILES: c1ccc(-c2cnc(-c3ccc(-c4nc(-c5ccccc5)cc(-c5ccc(-c6ccc(-c7ccc8sc9ccccc9c8c7)c7oc8ccccc8c67)cc5)n4)cc3)nc2)cc1 ; c1ccc(-c2cc(-c3ccc(-c4ccc(-c5ccc6sc7ccccc7c6c5)c5oc6ccccc6c45)cc3)nc(-c3ccc(-c4cnc(-c5ccccc5)nc4)cc3)n2)cc1 ; c1ccc(-c2cc(-c3ccc(-c4ccc(-c5ccc6sc7ccccc7c6c5)c5oc6ccccc6c45)cc3)nc(-c3ccc(-c4ccnc5ccccc45)cc3)n2)cc1 ; c1ccc(-c2cc(-c3ccc(-c4ccc(-c5ccc6sc7ccccc7c6c5)c5oc6ccccc6c45)cc3)nc(-c3ccc(-c4cccc5ncccc45)cc3)n2)cc1]

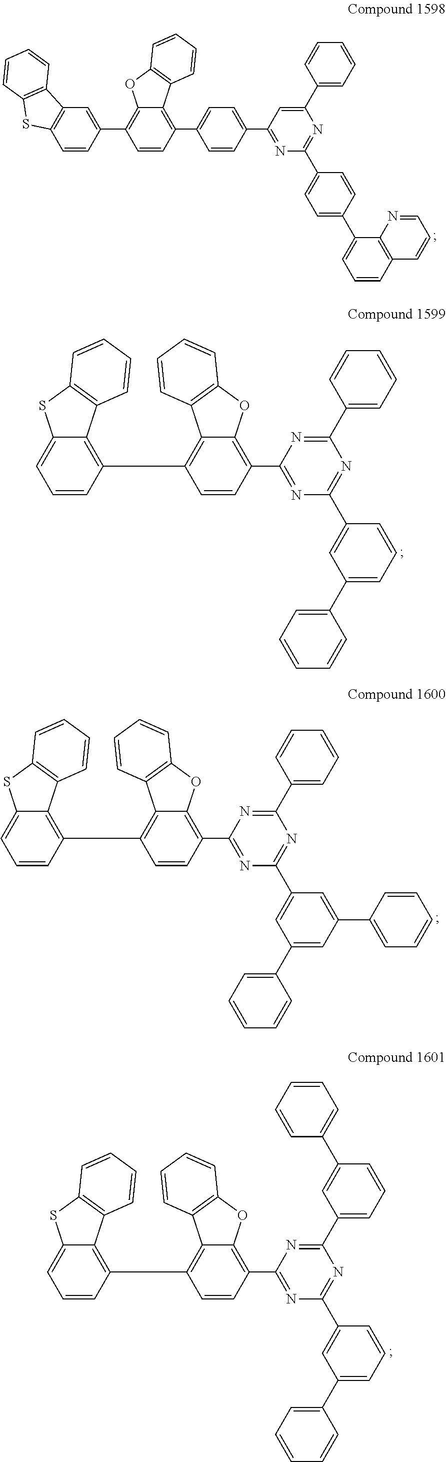 [SMILES: c1ccc(-c2cc(-c3ccc(-c4ccc(-c5ccc6sc7ccccc7c6c5)c5oc6ccccc6c45)cc3)nc(-c3ccc(-c4cccc5cccnc45)cc3)n2)cc1 ; c1ccc(-c2cccc(-c3nc(-c4ccccc4)nc(-c4ccc(-c5cccc6sc7ccccc7c56)c5c4oc4ccccc45)n3)c2)cc1 ; c1ccc(-c2cc(-c3ccccc3)cc(-c3nc(-c4ccccc4)nc(-c4ccc(-c5cccc6sc7ccccc7c56)c5c4oc4ccccc45)n3)c2)cc1 ; c1ccc(-c2cccc(-c3nc(-c4cccc(-c5ccccc5)c4)nc(-c4ccc(-c5cccc6sc7ccccc7c56)c5c4oc4ccccc45)n3)c2)cc1]

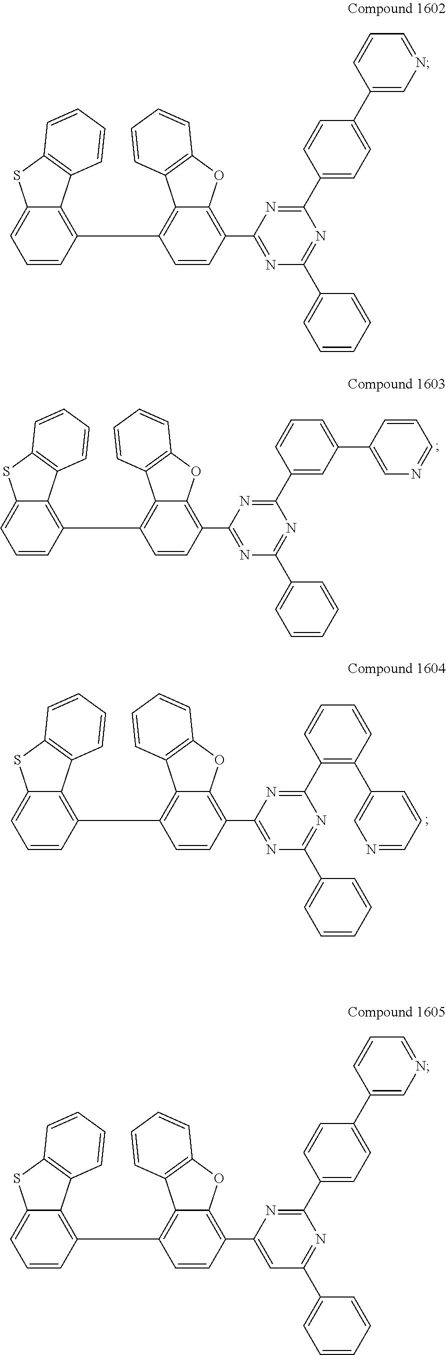 [SMILES: c1ccc(-c2nc(-c3ccc(-c4cccnc4)cc3)nc(-c3ccc(-c4cccc5sc6ccccc6c45)c4c3oc3ccccc34)n2)cc1 ; c1ccc(-c2nc(-c3cccc(-c4cccnc4)c3)nc(-c3ccc(-c4cccc5sc6ccccc6c45)c4c3oc3ccccc34)n2)cc1 ; c1ccc(-c2nc(-c3ccccc3-c3cccnc3)nc(-c3ccc(-c4cccc5sc6ccccc6c45)c4c3oc3ccccc34)n2)cc1 ; c1ccc(-c2cc(-c3ccc(-c4cccc5sc6ccccc6c45)c4c3oc3ccccc34)nc(-c3ccc(-c4cccnc4)cc3)n2)cc1]

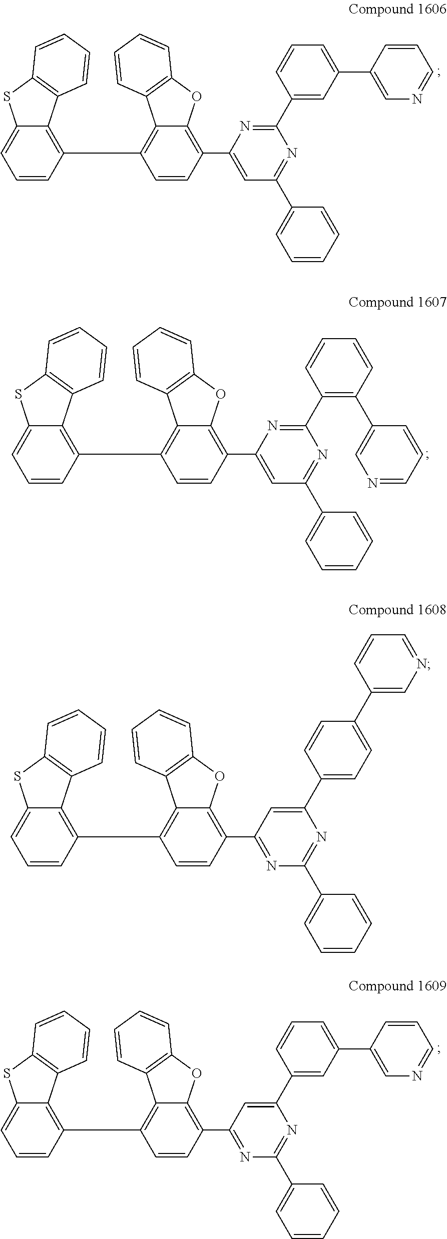 [SMILES: c1ccc(-c2cc(-c3ccc(-c4cccc5sc6ccccc6c45)c4c3oc3ccccc34)nc(-c3cccc(-c4cccnc4)c3)n2)cc1 ; c1ccc(-c2cc(-c3ccc(-c4cccc5sc6ccccc6c45)c4c3oc3ccccc34)nc(-c3ccccc3-c3cccnc3)n2)cc1 ; c1ccc(-c2nc(-c3ccc(-c4cccnc4)cc3)cc(-c3ccc(-c4cccc5sc6ccccc6c45)c4c3oc3ccccc34)n2)cc1 ; c1ccc(-c2nc(-c3cccc(-c4cccnc4)c3)cc(-c3ccc(-c4cccc5sc6ccccc6c45)c4c3oc3ccccc34)n2)cc1]

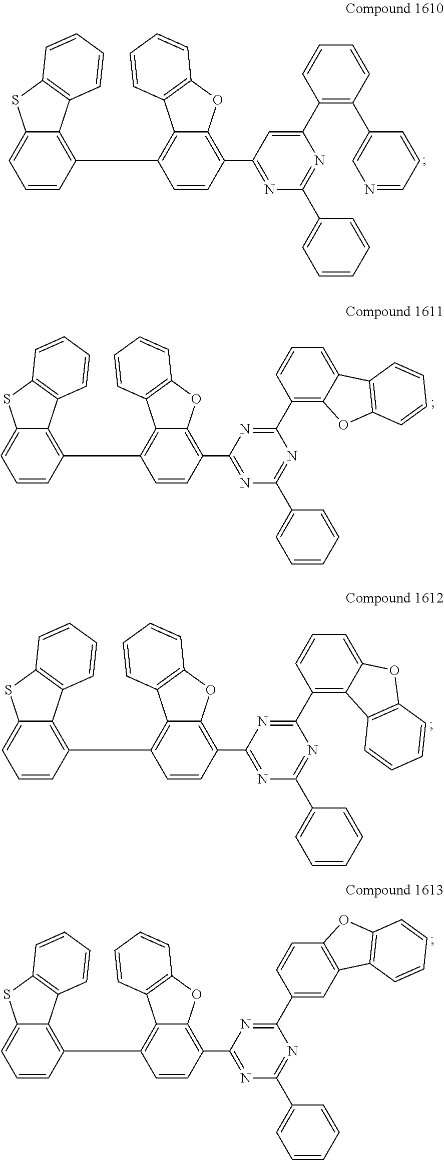 [SMILES: c1ccc(-c2nc(-c3ccccc3-c3cccnc3)cc(-c3ccc(-c4cccc5sc6ccccc6c45)c4c3oc3ccccc34)n2)cc1 ; c1ccc(-c2nc(-c3cccc4c3oc3ccccc34)nc(-c3ccc(-c4cccc5sc6ccccc6c45)c4c3oc3ccccc34)n2)cc1 ; c1ccc(-c2nc(-c3ccc(-c4cccc5sc6ccccc6c45)c4c3oc3ccccc34)nc(-c3cccc4oc5ccccc5c34)n2)cc1 ; c1ccc(-c2nc(-c3ccc4oc5ccccc5c4c3)nc(-c3ccc(-c4cccc5sc6ccccc6c45)c4c3oc3ccccc34)n2)cc1]

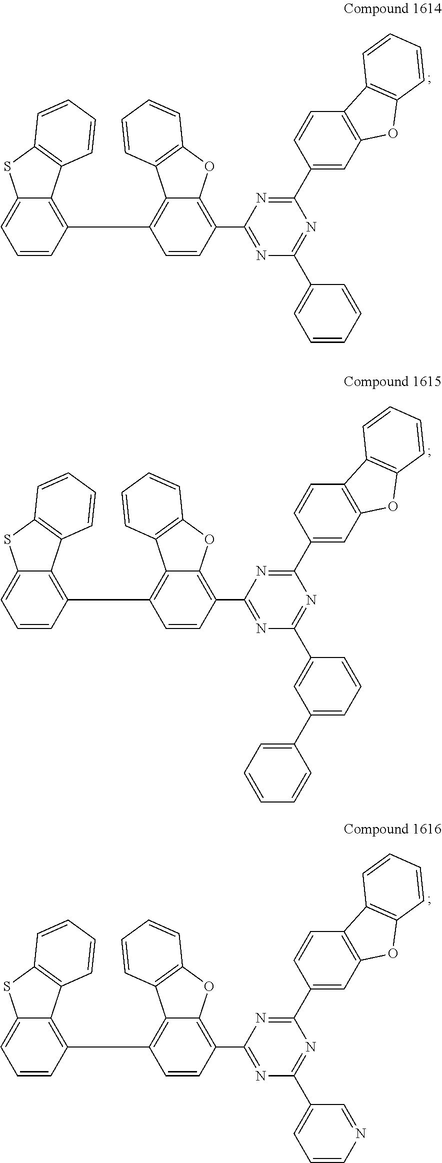 [SMILES: c1ccc(-c2nc(-c3ccc4c(c3)oc3ccccc34)nc(-c3ccc(-c4cccc5sc6ccccc6c45)c4c3oc3ccccc34)n2)cc1 ; c1ccc(-c2cccc(-c3nc(-c4ccc5c(c4)oc4ccccc45)nc(-c4ccc(-c5cccc6sc7ccccc7c56)c5c4oc4ccccc45)n3)c2)cc1 ; c1cncc(-c2nc(-c3ccc4c(c3)oc3ccccc34)nc(-c3ccc(-c4cccc5sc6ccccc6c45)c4c3oc3ccccc34)n2)c1]

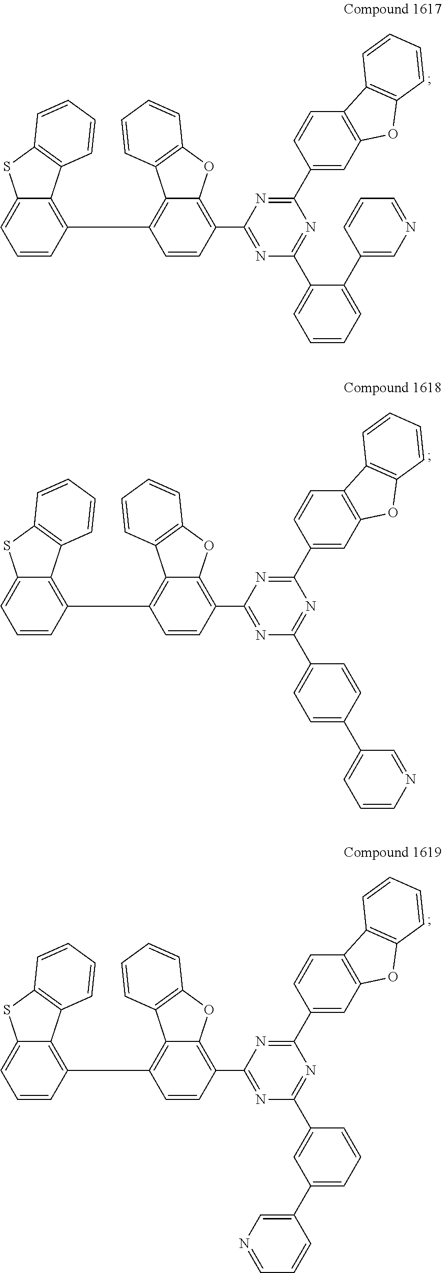 [SMILES: c1cncc(-c2ccccc2-c2nc(-c3ccc4c(c3)oc3ccccc34)nc(-c3ccc(-c4cccc5sc6ccccc6c45)c4c3oc3ccccc34)n2)c1 ; c1cncc(-c2ccc(-c3nc(-c4ccc5c(c4)oc4ccccc45)nc(-c4ccc(-c5cccc6sc7ccccc7c56)c5c4oc4ccccc45)n3)cc2)c1 ; c1cncc(-c2cccc(-c3nc(-c4ccc5c(c4)oc4ccccc45)nc(-c4ccc(-c5cccc6sc7ccccc7c56)c5c4oc4ccccc45)n3)c2)c1]

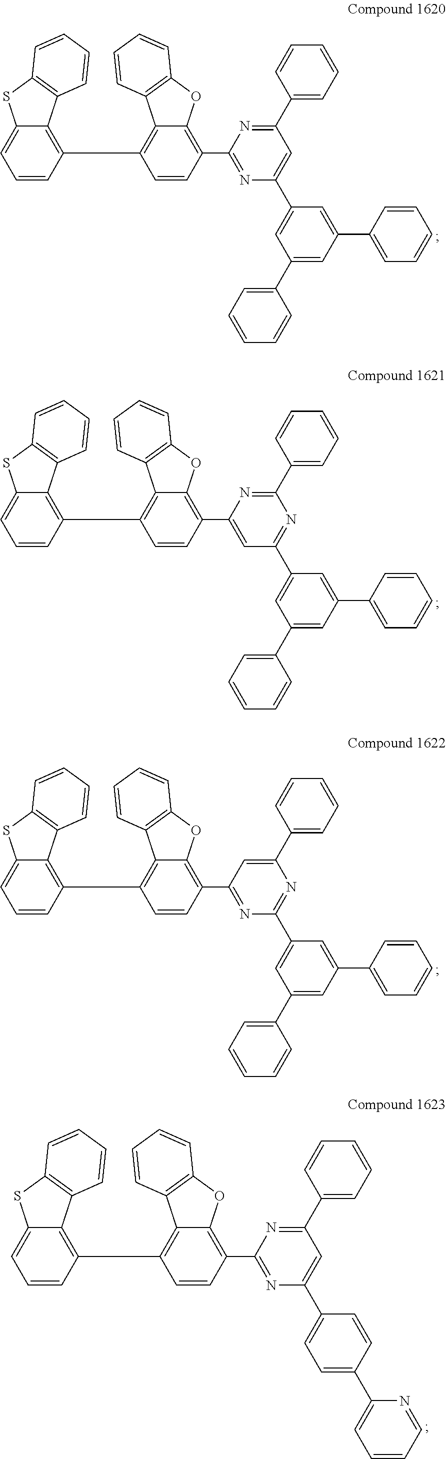 [SMILES: c1ccc(-c2cc(-c3ccccc3)cc(-c3cc(-c4ccccc4)nc(-c4ccc(-c5cccc6sc7ccccc7c56)c5c4oc4ccccc45)n3)c2)cc1 ; c1ccc(-c2cc(-c3ccccc3)cc(-c3cc(-c4ccc(-c5cccc6sc7ccccc7c56)c5c4oc4ccccc45)nc(-c4ccccc4)n3)c2)cc1 ; c1ccc(-c2cc(-c3ccccc3)cc(-c3nc(-c4ccccc4)cc(-c4ccc(-c5cccc6sc7ccccc7c56)c5c4oc4ccccc45)n3)c2)cc1 ; c1ccc(-c2cc(-c3ccc(-c4ccccn4)cc3)nc(-c3ccc(-c4cccc5sc6ccccc6c45)c4c3oc3ccccc34)n2)cc1]

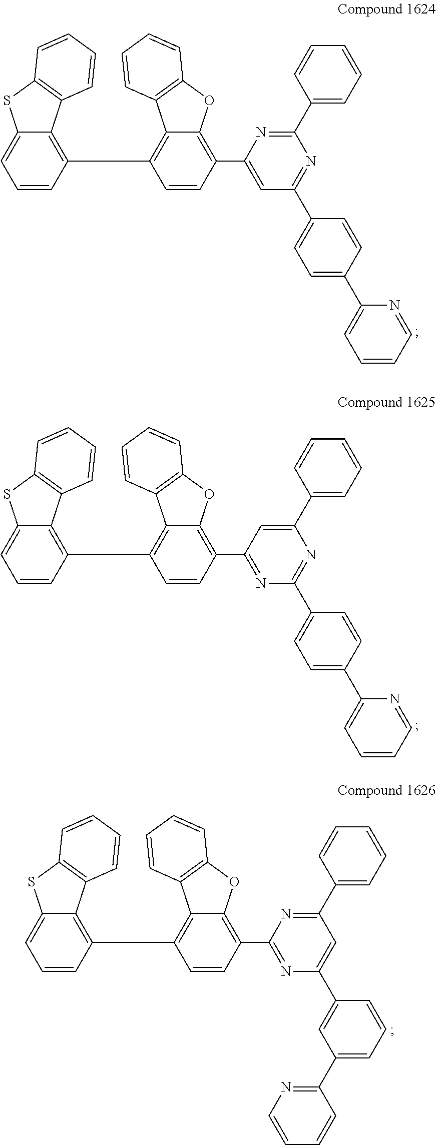 [SMILES: c1ccc(-c2nc(-c3ccc(-c4ccccn4)cc3)cc(-c3ccc(-c4cccc5sc6ccccc6c45)c4c3oc3ccccc34)n2)cc1 ; c1ccc(-c2cc(-c3ccc(-c4cccc5sc6ccccc6c45)c4c3oc3ccccc34)nc(-c3ccc(-c4ccccn4)cc3)n2)cc1 ; c1ccc(-c2cc(-c3cccc(-c4ccccn4)c3)nc(-c3ccc(-c4cccc5sc6ccccc6c45)c4c3oc3ccccc34)n2)cc1]

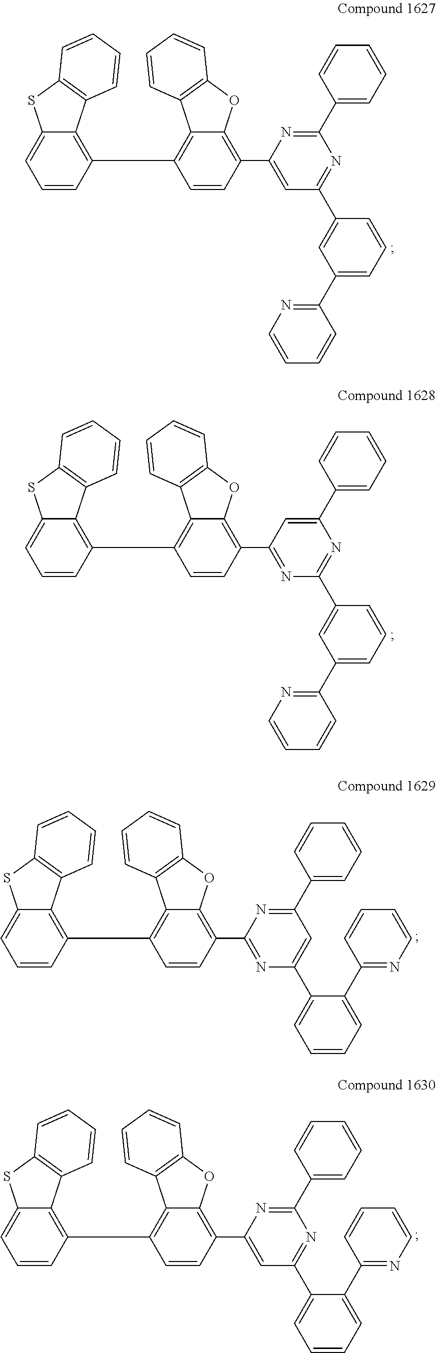 [SMILES: c1ccc(-c2nc(-c3cccc(-c4ccccn4)c3)cc(-c3ccc(-c4cccc5sc6ccccc6c45)c4c3oc3ccccc34)n2)cc1 ; c1ccc(-c2cc(-c3ccc(-c4cccc5sc6ccccc6c45)c4c3oc3ccccc34)nc(-c3cccc(-c4ccccn4)c3)n2)cc1 ; c1ccc(-c2cc(-c3ccccc3-c3ccccn3)nc(-c3ccc(-c4cccc5sc6ccccc6c45)c4c3oc3ccccc34)n2)cc1 ; c1ccc(-c2nc(-c3ccccc3-c3ccccn3)cc(-c3ccc(-c4cccc5sc6ccccc6c45)c4c3oc3ccccc34)n2)cc1]

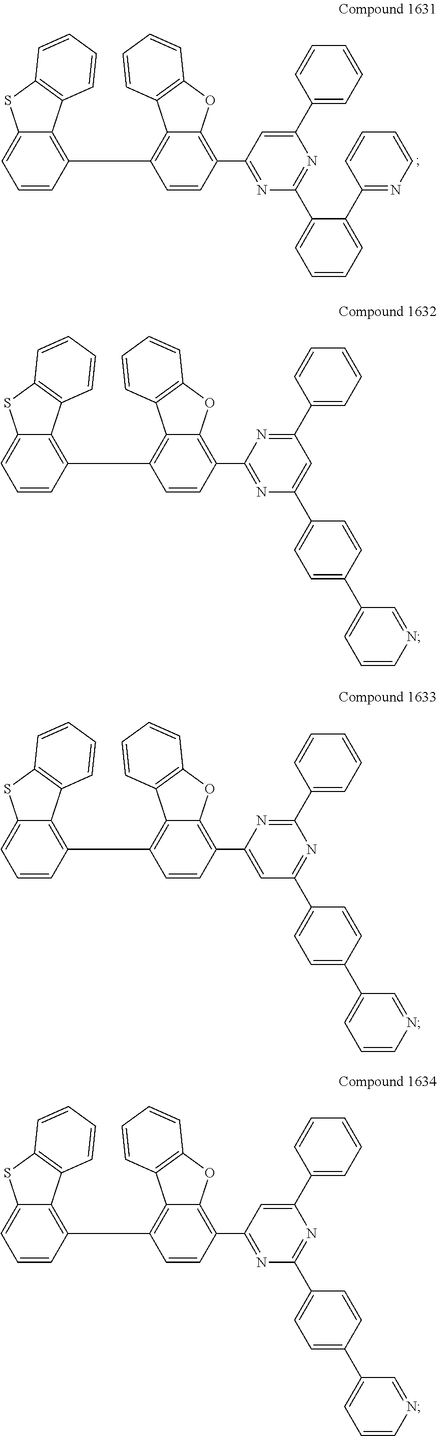 [SMILES: c1ccc(-c2cc(-c3ccc(-c4cccc5sc6ccccc6c45)c4c3oc3ccccc34)nc(-c3ccccc3-c3ccccn3)n2)cc1 ; c1ccc(-c2cc(-c3ccc(-c4cccnc4)cc3)nc(-c3ccc(-c4cccc5sc6ccccc6c45)c4c3oc3ccccc34)n2)cc1 ; c1ccc(-c2nc(-c3ccc(-c4cccnc4)cc3)cc(-c3ccc(-c4cccc5sc6ccccc6c45)c4c3oc3ccccc34)n2)cc1 ; c1ccc(-c2cc(-c3ccc(-c4cccc5sc6ccccc6c45)c4c3oc3ccccc34)nc(-c3ccc(-c4cccnc4)cc3)n2)cc1]

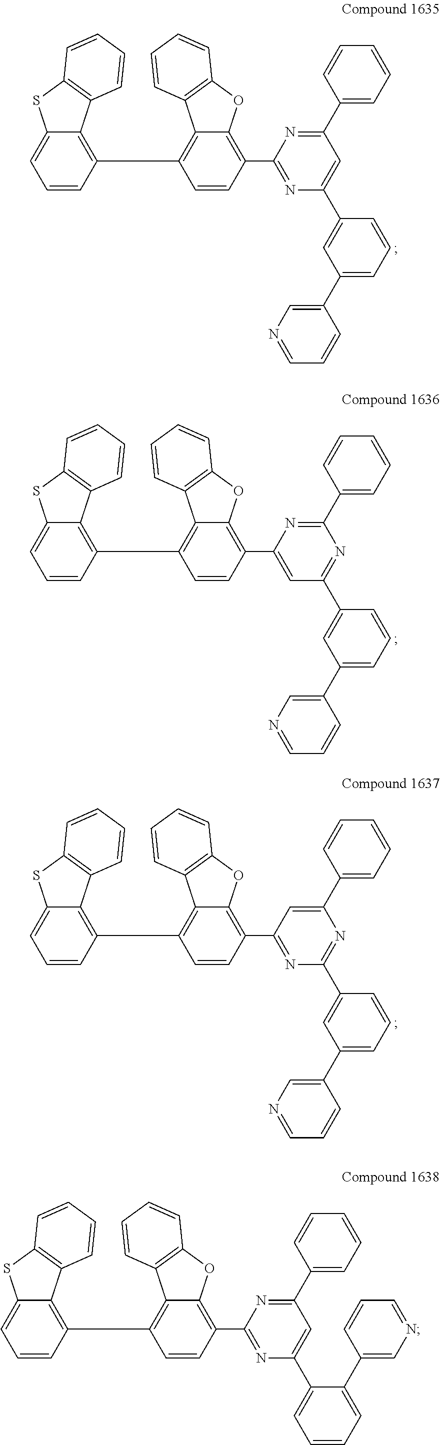 [SMILES: c1ccc(-c2cc(-c3cccc(-c4cccnc4)c3)nc(-c3ccc(-c4cccc5sc6ccccc6c45)c4c3oc3ccccc34)n2)cc1 ; c1ccc(-c2nc(-c3cccc(-c4cccnc4)c3)cc(-c3ccc(-c4cccc5sc6ccccc6c45)c4c3oc3ccccc34)n2)cc1 ; c1ccc(-c2cc(-c3ccc(-c4cccc5sc6ccccc6c45)c4c3oc3ccccc34)nc(-c3cccc(-c4cccnc4)c3)n2)cc1 ; c1ccc(-c2cc(-c3ccccc3-c3cccnc3)nc(-c3ccc(-c4cccc5sc6ccccc6c45)c4c3oc3ccccc34)n2)cc1]

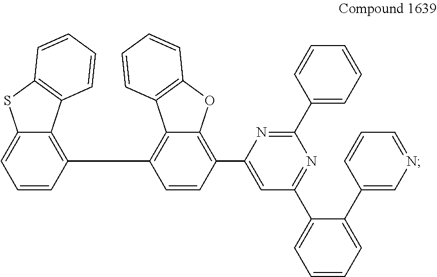 [SMILES: c1ccc(-c2nc(-c3ccccc3-c3cccnc3)cc(-c3ccc(-c4cccc5sc6ccccc6c45)c4c3oc3ccccc34)n2)cc1]

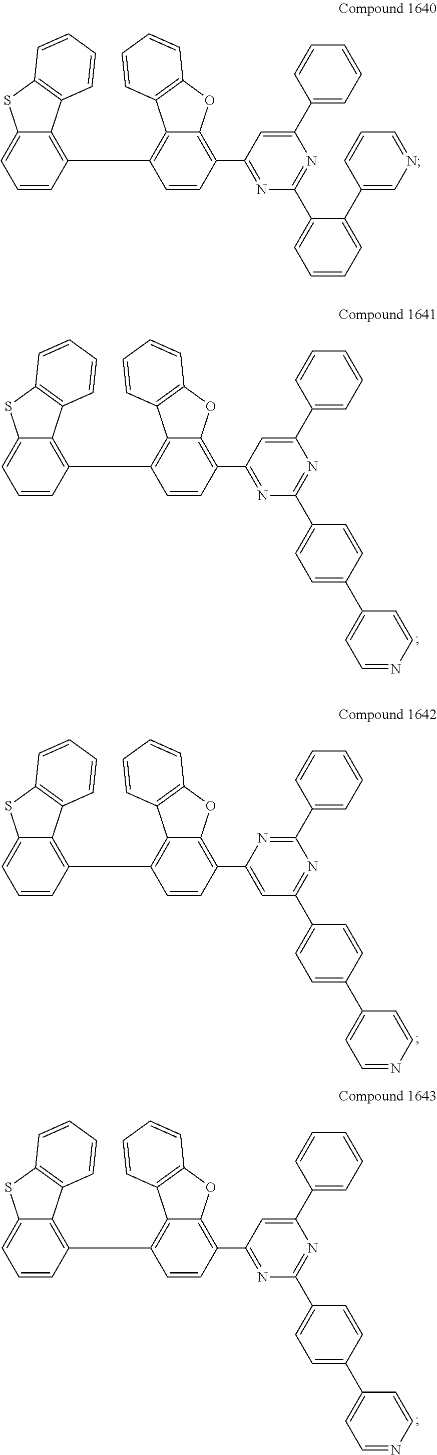 [SMILES: c1ccc(-c2cc(-c3ccc(-c4cccc5sc6ccccc6c45)c4c3oc3ccccc34)nc(-c3ccccc3-c3cccnc3)n2)cc1 ; c1ccc(-c2cc(-c3ccc(-c4cccc5sc6ccccc6c45)c4c3oc3ccccc34)nc(-c3ccc(-c4ccncc4)cc3)n2)cc1 ; c1ccc(-c2nc(-c3ccc(-c4ccncc4)cc3)cc(-c3ccc(-c4cccc5sc6ccccc6c45)c4c3oc3ccccc34)n2)cc1 ; c1ccc(-c2cc(-c3ccc(-c4cccc5sc6ccccc6c45)c4c3oc3ccccc34)nc(-c3ccc(-c4ccncc4)cc3)n2)cc1]

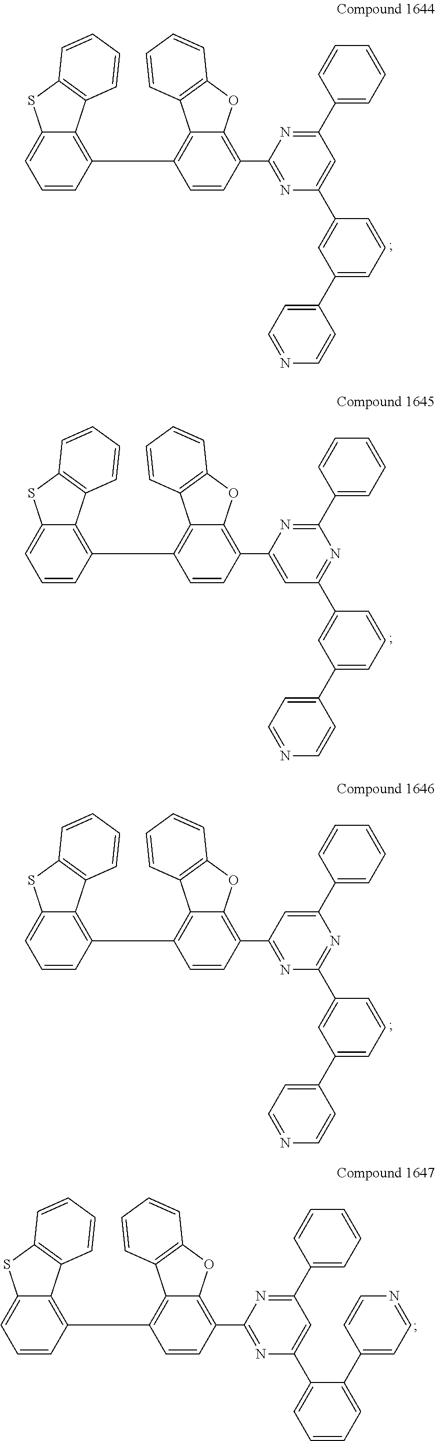 [SMILES: c1ccc(-c2cc(-c3cccc(-c4ccncc4)c3)nc(-c3ccc(-c4cccc5sc6ccccc6c45)c4c3oc3ccccc34)n2)cc1 ; c1ccc(-c2nc(-c3cccc(-c4ccncc4)c3)cc(-c3ccc(-c4cccc5sc6ccccc6c45)c4c3oc3ccccc34)n2)cc1 ; c1ccc(-c2cc(-c3ccc(-c4cccc5sc6ccccc6c45)c4c3oc3ccccc34)nc(-c3cccc(-c4ccncc4)c3)n2)cc1 ; c1ccc(-c2cc(-c3ccccc3-c3ccncc3)nc(-c3ccc(-c4cccc5sc6ccccc6c45)c4c3oc3ccccc34)n2)cc1]

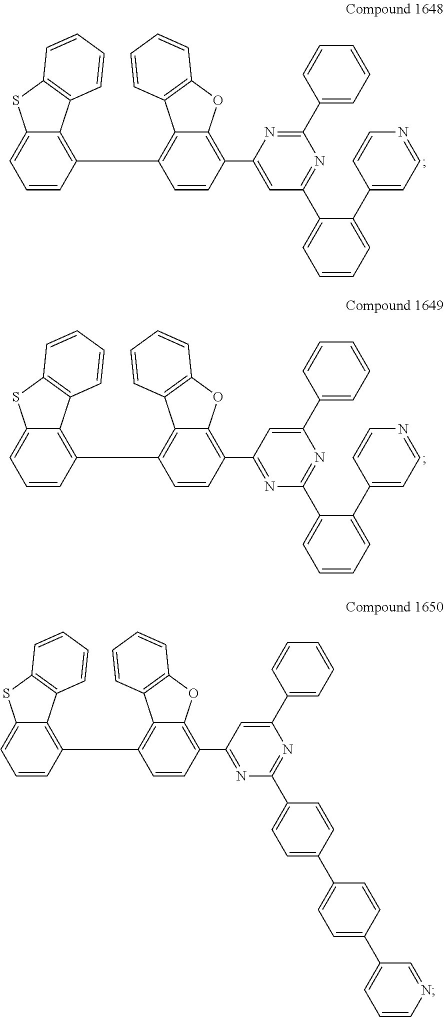 [SMILES: c1ccc(-c2nc(-c3ccccc3-c3ccncc3)cc(-c3ccc(-c4cccc5sc6ccccc6c45)c4c3oc3ccccc34)n2)cc1 ; c1ccc(-c2cc(-c3ccc(-c4cccc5sc6ccccc6c45)c4c3oc3ccccc34)nc(-c3ccccc3-c3ccncc3)n2)cc1 ; c1ccc(-c2cc(-c3ccc(-c4cccc5sc6ccccc6c45)c4c3oc3ccccc34)nc(-c3ccc(-c4ccc(-c5cccnc5)cc4)cc3)n2)cc1]

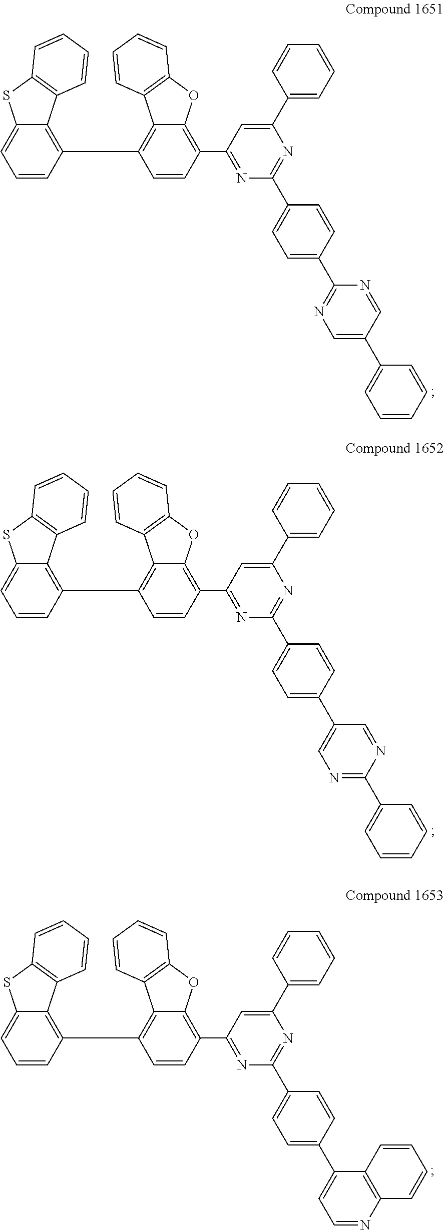 [SMILES: c1ccc(-c2cnc(-c3ccc(-c4nc(-c5ccccc5)cc(-c5ccc(-c6cccc7sc8ccccc8c67)c6c5oc5ccccc56)n4)cc3)nc2)cc1 ; c1ccc(-c2cc(-c3ccc(-c4cccc5sc6ccccc6c45)c4c3oc3ccccc34)nc(-c3ccc(-c4cnc(-c5ccccc5)nc4)cc3)n2)cc1 ; c1ccc(-c2cc(-c3ccc(-c4cccc5sc6ccccc6c45)c4c3oc3ccccc34)nc(-c3ccc(-c4ccnc5ccccc45)cc3)n2)cc1]

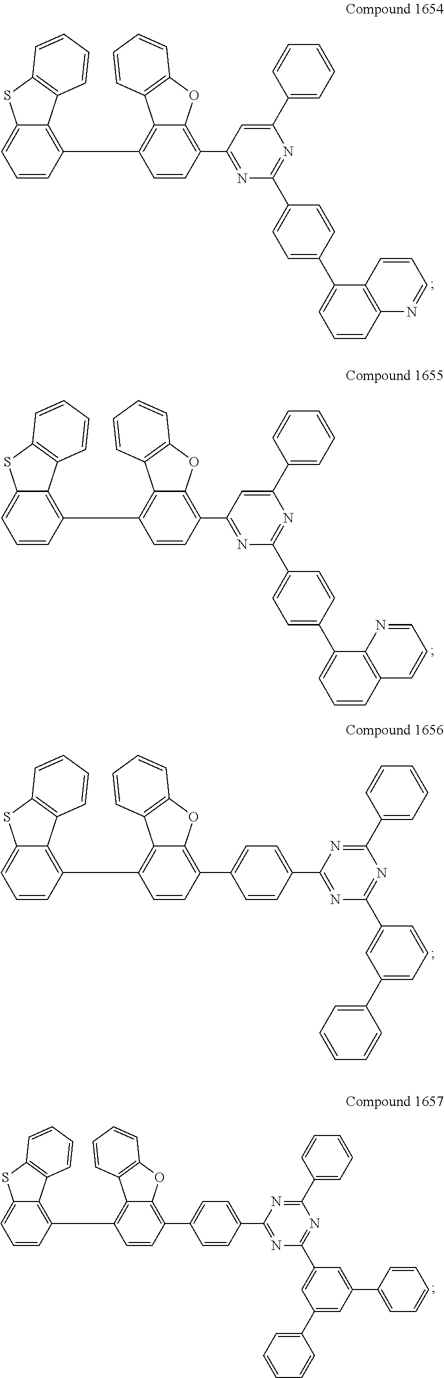 [SMILES: c1ccc(-c2cc(-c3ccc(-c4cccc5sc6ccccc6c45)c4c3oc3ccccc34)nc(-c3ccc(-c4cccc5ncccc45)cc3)n2)cc1 ; c1ccc(-c2cc(-c3ccc(-c4cccc5sc6ccccc6c45)c4c3oc3ccccc34)nc(-c3ccc(-c4cccc5cccnc45)cc3)n2)cc1 ; c1ccc(-c2cccc(-c3nc(-c4ccccc4)nc(-c4ccc(-c5ccc(-c6cccc7sc8ccccc8c67)c6c5oc5ccccc56)cc4)n3)c2)cc1 ; c1ccc(-c2cc(-c3ccccc3)cc(-c3nc(-c4ccccc4)nc(-c4ccc(-c5ccc(-c6cccc7sc8ccccc8c67)c6c5oc5ccccc56)cc4)n3)c2)cc1]

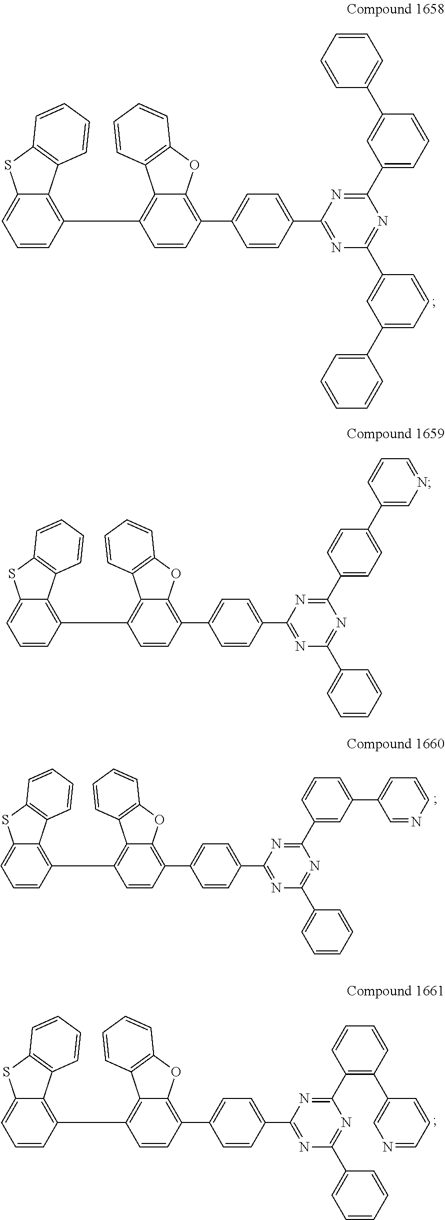 [SMILES: c1ccc(-c2cccc(-c3nc(-c4ccc(-c5ccc(-c6cccc7sc8ccccc8c67)c6c5oc5ccccc56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5cccc6sc7ccccc7c56)c5c4oc4ccccc45)cc3)n2)cc1 ; c1ccc(-c2nc(-c3ccc(-c4ccc(-c5cccc6sc7ccccc7c56)c5c4oc4ccccc45)cc3)nc(-c3cccc(-c4cccnc4)c3)n2)cc1 ; c1ccc(-c2nc(-c3ccc(-c4ccc(-c5cccc6sc7ccccc7c56)c5c4oc4ccccc45)cc3)nc(-c3ccccc3-c3cccnc3)n2)cc1]

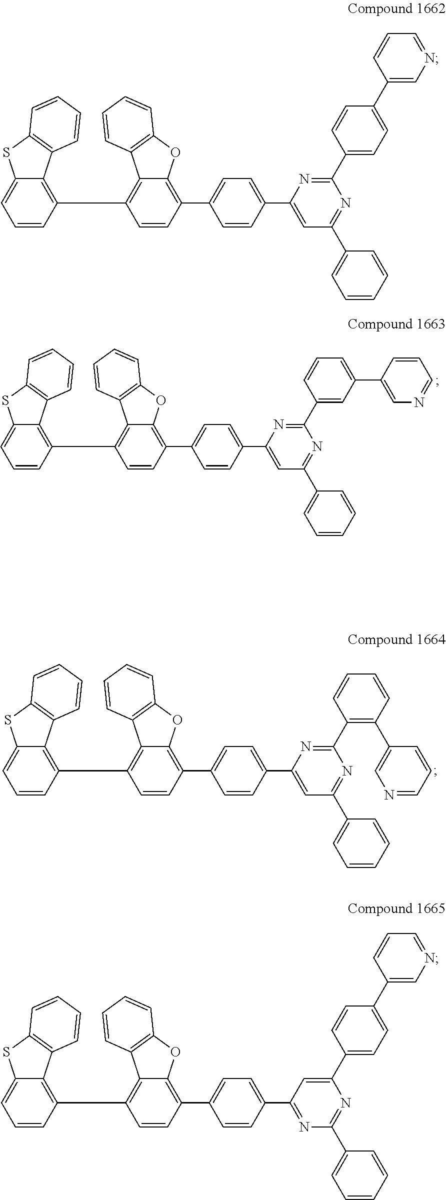 [SMILES: c1ccc(-c2cc(-c3ccc(-c4ccc(-c5cccc6sc7ccccc7c56)c5c4oc4ccccc45)cc3)nc(-c3ccc(-c4cccnc4)cc3)n2)cc1 ; c1ccc(-c2cc(-c3ccc(-c4ccc(-c5cccc6sc7ccccc7c56)c5c4oc4ccccc45)cc3)nc(-c3cccc(-c4cccnc4)c3)n2)cc1 ; c1ccc(-c2cc(-c3ccc(-c4ccc(-c5cccc6sc7ccccc7c56)c5c4oc4ccccc45)cc3)nc(-c3ccccc3-c3cccnc3)n2)cc1 ; c1ccc(-c2nc(-c3ccc(-c4cccnc4)cc3)cc(-c3ccc(-c4ccc(-c5cccc6sc7ccccc7c56)c5c4oc4ccccc45)cc3)n2)cc1]

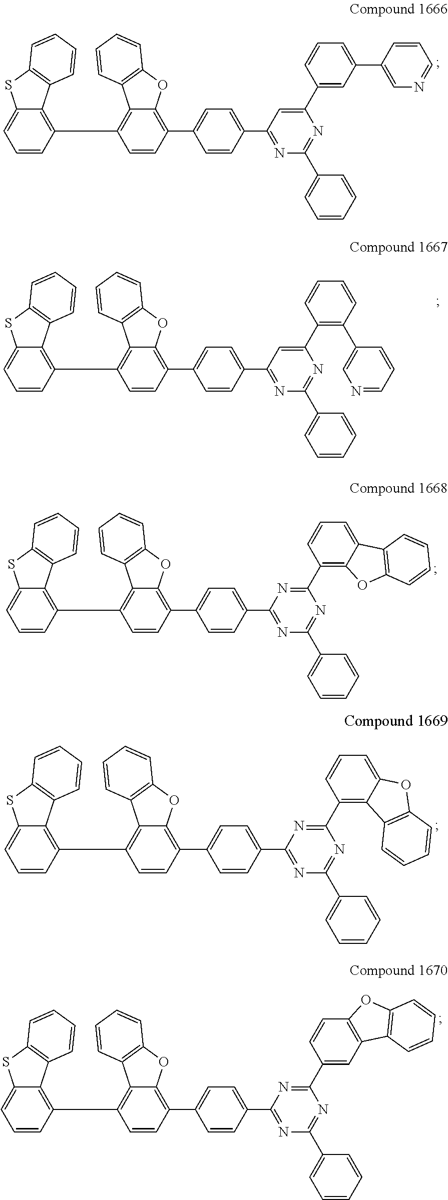 [SMILES: c1ccc(-c2nc(-c3ccc(-c4ccc(-c5cccc6sc7ccccc7c56)c5c4oc4ccccc45)cc3)cc(-c3cccc(-c4cccnc4)c3)n2)cc1 ; c1ccc(-c2nc(-c3ccc(-c4ccc(-c5cccc6sc7ccccc7c56)c5c4oc4ccccc45)cc3)cc(-c3ccccc3-c3cccnc3)n2)cc1 ; c1ccc(-c2nc(-c3ccc(-c4ccc(-c5cccc6sc7ccccc7c56)c5c4oc4ccccc45)cc3)nc(-c3cccc4c3oc3ccccc34)n2)cc1 ; c1ccc(-c2nc(-c3ccc(-c4ccc(-c5cccc6sc7ccccc7c56)c5c4oc4ccccc45)cc3)nc(-c3cccc4oc5ccccc5c34)n2)cc1 ; c1ccc(-c2nc(-c3ccc(-c4ccc(-c5cccc6sc7ccccc7c56)c5c4oc4ccccc45)cc3)nc(-c3ccc4oc5ccccc5c4c3)n2)cc1]

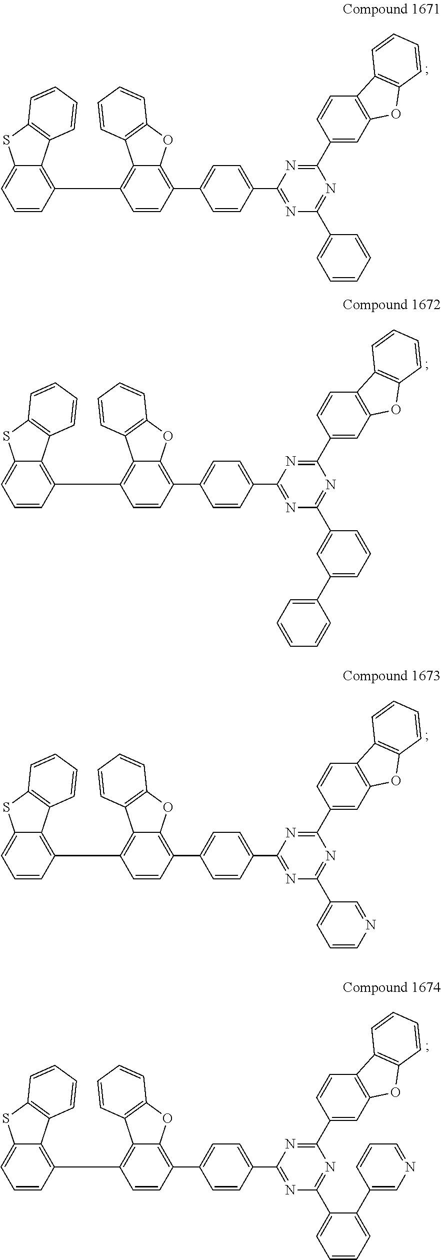 [SMILES: c1ccc(-c2nc(-c3ccc(-c4ccc(-c5cccc6sc7ccccc7c56)c5c4oc4ccccc45)cc3)nc(-c3ccc4c(c3)oc3ccccc34)n2)cc1 ; c1ccc(-c2cccc(-c3nc(-c4ccc(-c5ccc(-c6cccc7sc8ccccc8c67)c6c5oc5ccccc56)cc4)nc(-c4ccc5c(c4)oc4ccccc45)n3)c2)cc1 ; c1cncc(-c2nc(-c3ccc(-c4ccc(-c5cccc6sc7ccccc7c56)c5c4oc4ccccc45)cc3)nc(-c3ccc4c(c3)oc3ccccc34)n2)c1 ; c1cncc(-c2ccccc2-c2nc(-c3ccc(-c4ccc(-c5cccc6sc7ccccc7c56)c5c4oc4ccccc45)cc3)nc(-c3ccc4c(c3)oc3ccccc34)n2)c1]

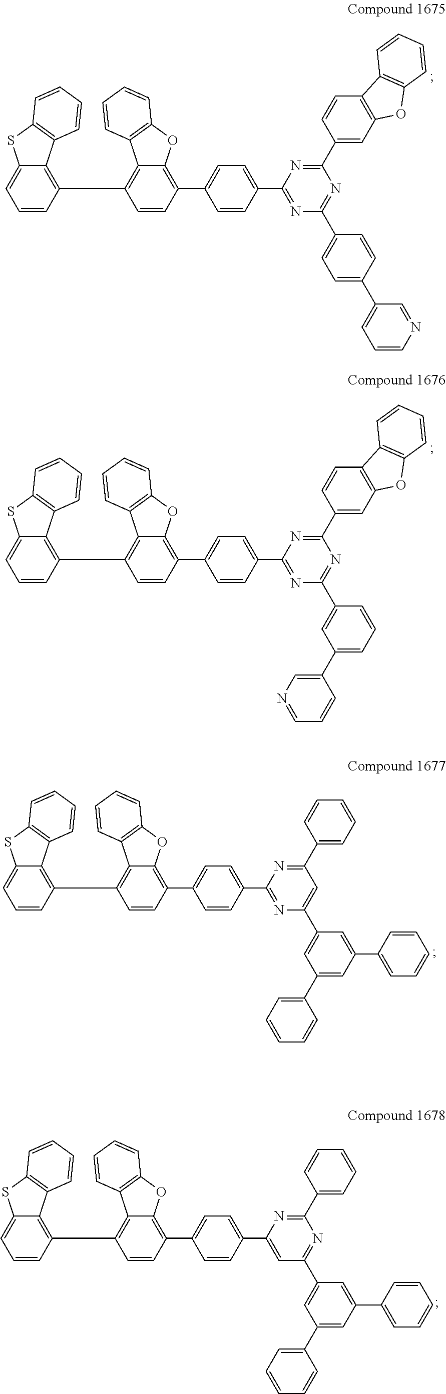 [SMILES: c1cncc(-c2ccc(-c3nc(-c4ccc(-c5ccc(-c6cccc7sc8ccccc8c67)c6c5oc5ccccc56)cc4)nc(-c4ccc5c(c4)oc4ccccc45)n3)cc2)c1 ; c1cncc(-c2cccc(-c3nc(-c4ccc(-c5ccc(-c6cccc7sc8ccccc8c67)c6c5oc5ccccc56)cc4)nc(-c4ccc5c(c4)oc4ccccc45)n3)c2)c1 ; c1ccc(-c2cc(-c3ccccc3)cc(-c3cc(-c4ccccc4)nc(-c4ccc(-c5ccc(-c6cccc7sc8ccccc8c67)c6c5oc5ccccc56)cc4)n3)c2)cc1 ; c1ccc(-c2cc(-c3ccccc3)cc(-c3cc(-c4ccc(-c5ccc(-c6cccc7sc8ccccc8c67)c6c5oc5ccccc56)cc4)nc(-c4ccccc4)n3)c2)cc1]

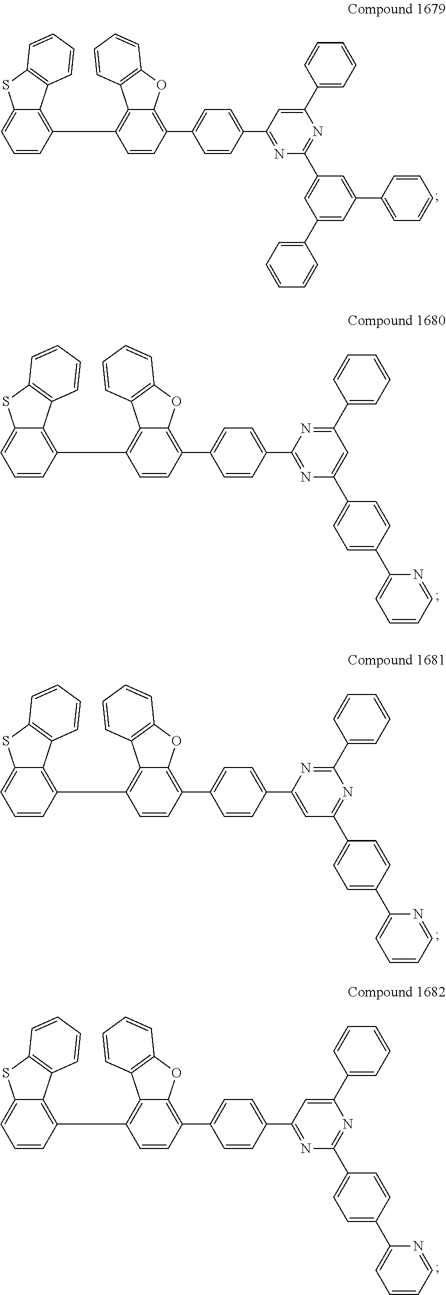 [SMILES: c1ccc(-c2cc(-c3ccccc3)cc(-c3nc(-c4ccccc4)cc(-c4ccc(-c5ccc(-c6cccc7sc8ccccc8c67)c6c5oc5ccccc56)cc4)n3)c2)cc1 ; c1ccc(-c2cc(-c3ccc(-c4ccccn4)cc3)nc(-c3ccc(-c4ccc(-c5cccc6sc7ccccc7c56)c5c4oc4ccccc45)cc3)n2)cc1 ; c1ccc(-c2nc(-c3ccc(-c4ccccn4)cc3)cc(-c3ccc(-c4ccc(-c5cccc6sc7ccccc7c56)c5c4oc4ccccc45)cc3)n2)cc1 ; c1ccc(-c2cc(-c3ccc(-c4ccc(-c5cccc6sc7ccccc7c56)c5c4oc4ccccc45)cc3)nc(-c3ccc(-c4ccccn4)cc3)n2)cc1]

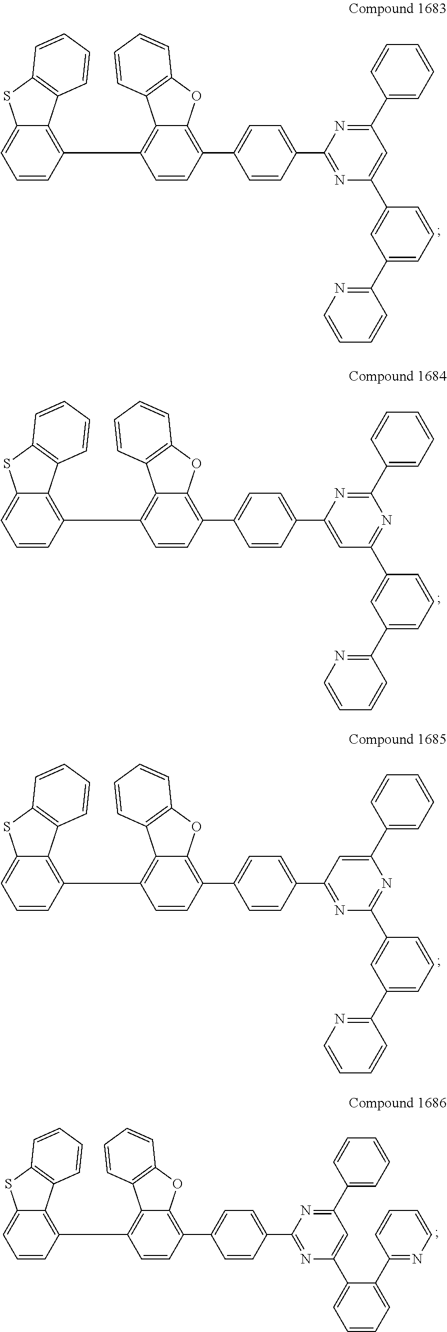 [SMILES: c1ccc(-c2cc(-c3cccc(-c4ccccn4)c3)nc(-c3ccc(-c4ccc(-c5cccc6sc7ccccc7c56)c5c4oc4ccccc45)cc3)n2)cc1 ; c1ccc(-c2nc(-c3ccc(-c4ccc(-c5cccc6sc7ccccc7c56)c5c4oc4ccccc45)cc3)cc(-c3cccc(-c4ccccn4)c3)n2)cc1 ; c1ccc(-c2cc(-c3ccc(-c4ccc(-c5cccc6sc7ccccc7c56)c5c4oc4ccccc45)cc3)nc(-c3cccc(-c4ccccn4)c3)n2)cc1 ; c1ccc(-c2cc(-c3ccccc3-c3ccccn3)nc(-c3ccc(-c4ccc(-c5cccc6sc7ccccc7c56)c5c4oc4ccccc45)cc3)n2)cc1]

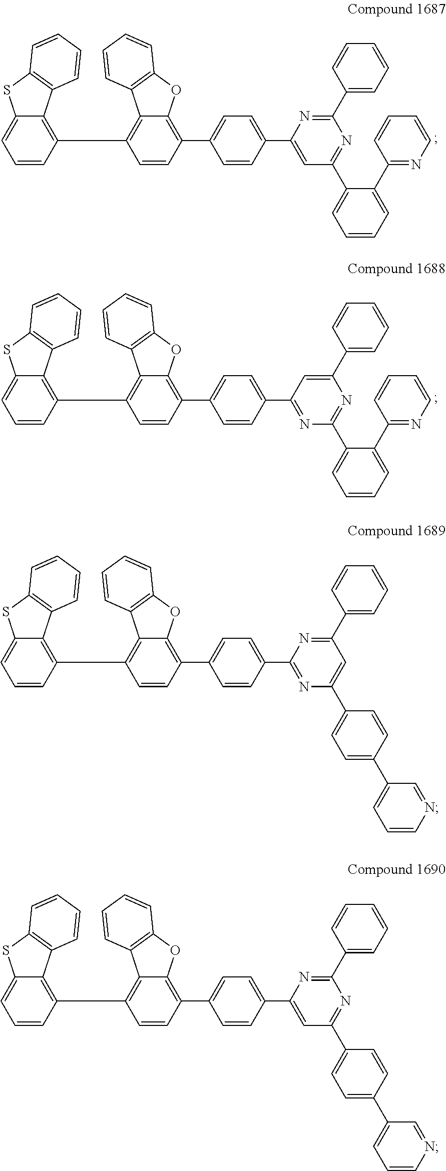 [SMILES: c1ccc(-c2nc(-c3ccc(-c4ccc(-c5cccc6sc7ccccc7c56)c5c4oc4ccccc45)cc3)cc(-c3ccccc3-c3ccccn3)n2)cc1 ; c1ccc(-c2cc(-c3ccc(-c4ccc(-c5cccc6sc7ccccc7c56)c5c4oc4ccccc45)cc3)nc(-c3ccccc3-c3ccccn3)n2)cc1 ; c1ccc(-c2cc(-c3ccc(-c4cccnc4)cc3)nc(-c3ccc(-c4ccc(-c5cccc6sc7ccccc7c56)c5c4oc4ccccc45)cc3)n2)cc1 ; c1ccc(-c2nc(-c3ccc(-c4cccnc4)cc3)cc(-c3ccc(-c4ccc(-c5cccc6sc7ccccc7c56)c5c4oc4ccccc45)cc3)n2)cc1]

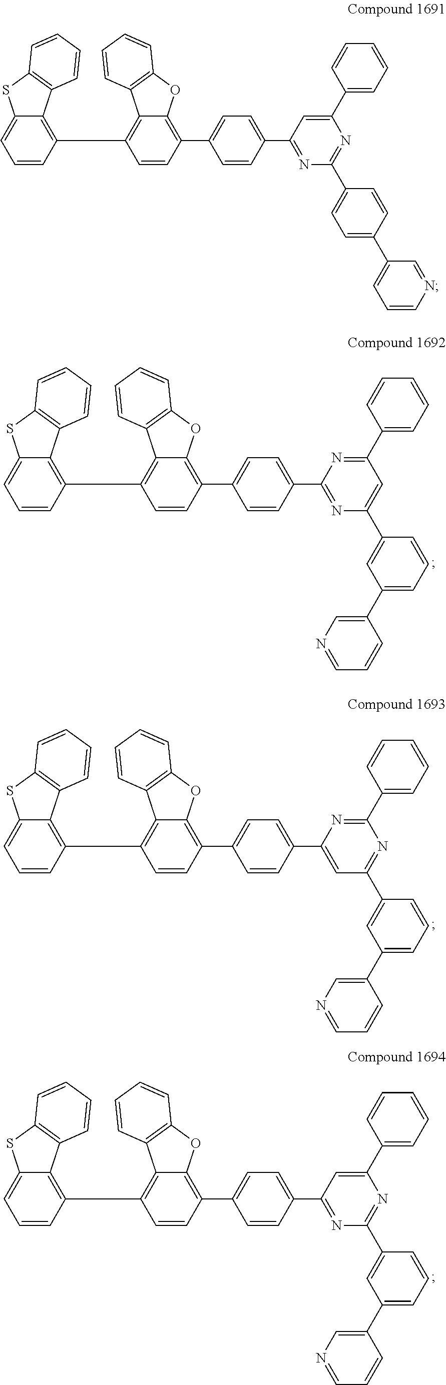 [SMILES: c1ccc(-c2cc(-c3ccc(-c4ccc(-c5cccc6sc7ccccc7c56)c5c4oc4ccccc45)cc3)nc(-c3ccc(-c4cccnc4)cc3)n2)cc1 ; c1ccc(-c2cc(-c3cccc(-c4cccnc4)c3)nc(-c3ccc(-c4ccc(-c5cccc6sc7ccccc7c56)c5c4oc4ccccc45)cc3)n2)cc1 ; c1ccc(-c2nc(-c3ccc(-c4ccc(-c5cccc6sc7ccccc7c56)c5c4oc4ccccc45)cc3)cc(-c3cccc(-c4cccnc4)c3)n2)cc1 ; c1ccc(-c2cc(-c3ccc(-c4ccc(-c5cccc6sc7ccccc7c56)c5c4oc4ccccc45)cc3)nc(-c3cccc(-c4cccnc4)c3)n2)cc1]

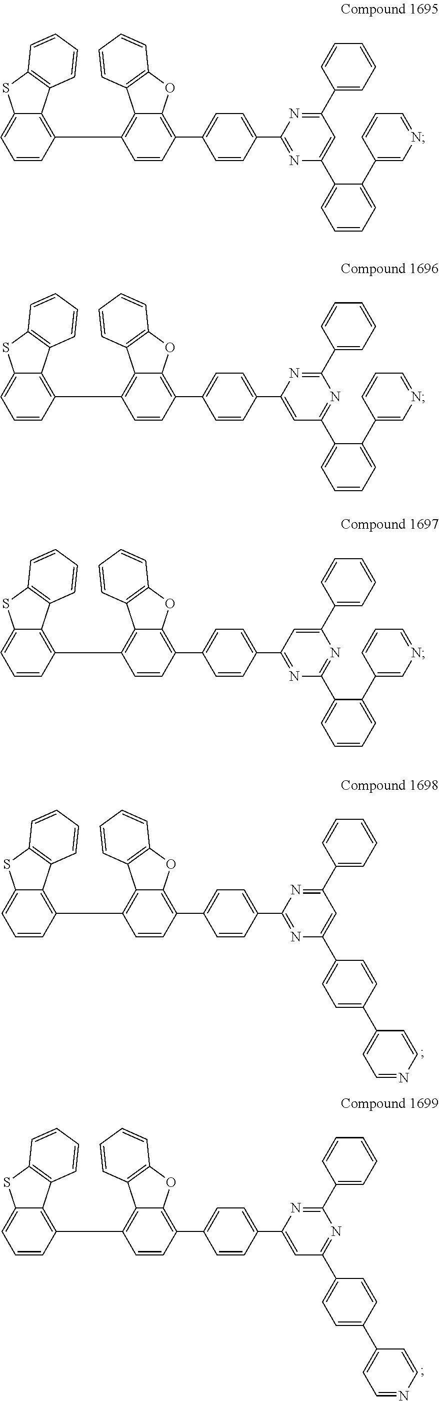 [SMILES: c1ccc(-c2cc(-c3ccccc3-c3cccnc3)nc(-c3ccc(-c4ccc(-c5cccc6sc7ccccc7c56)c5c4oc4ccccc45)cc3)n2)cc1 ; c1ccc(-c2nc(-c3ccc(-c4ccc(-c5cccc6sc7ccccc7c56)c5c4oc4ccccc45)cc3)cc(-c3ccccc3-c3cccnc3)n2)cc1 ; c1ccc(-c2cc(-c3ccc(-c4ccc(-c5cccc6sc7ccccc7c56)c5c4oc4ccccc45)cc3)nc(-c3ccccc3-c3cccnc3)n2)cc1 ; c1ccc(-c2cc(-c3ccc(-c4ccncc4)cc3)nc(-c3ccc(-c4ccc(-c5cccc6sc7ccccc7c56)c5c4oc4ccccc45)cc3)n2)cc1 ; c1ccc(-c2nc(-c3ccc(-c4ccncc4)cc3)cc(-c3ccc(-c4ccc(-c5cccc6sc7ccccc7c56)c5c4oc4ccccc45)cc3)n2)cc1]

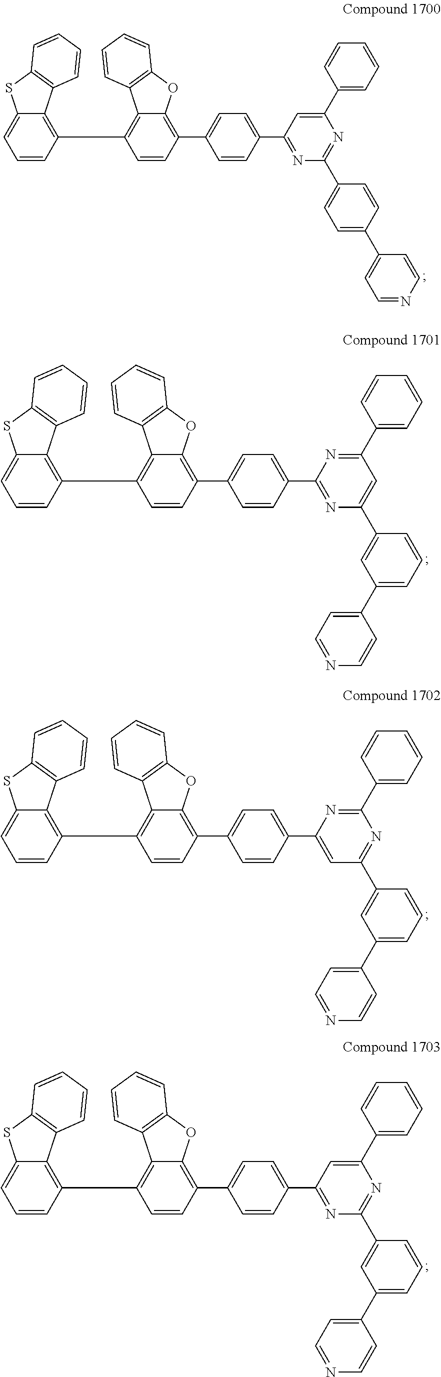 [SMILES: c1ccc(-c2cc(-c3ccc(-c4ccc(-c5cccc6sc7ccccc7c56)c5c4oc4ccccc45)cc3)nc(-c3ccc(-c4ccncc4)cc3)n2)cc1 ; c1ccc(-c2cc(-c3cccc(-c4ccncc4)c3)nc(-c3ccc(-c4ccc(-c5cccc6sc7ccccc7c56)c5c4oc4ccccc45)cc3)n2)cc1 ; c1ccc(-c2nc(-c3ccc(-c4ccc(-c5cccc6sc7ccccc7c56)c5c4oc4ccccc45)cc3)cc(-c3cccc(-c4ccncc4)c3)n2)cc1 ; c1ccc(-c2cc(-c3ccc(-c4ccc(-c5cccc6sc7ccccc7c56)c5c4oc4ccccc45)cc3)nc(-c3cccc(-c4ccncc4)c3)n2)cc1]

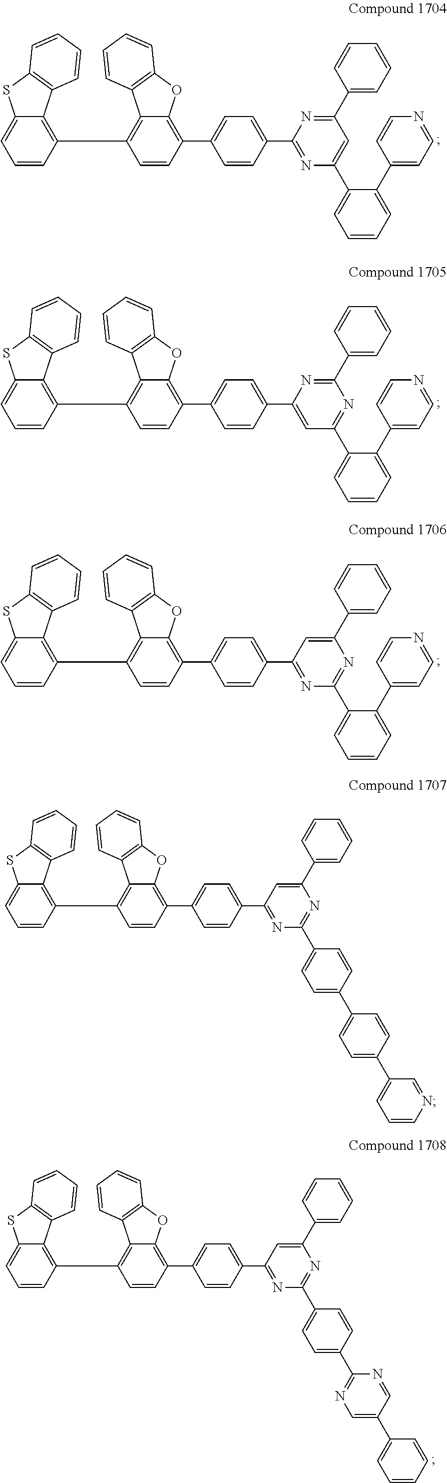

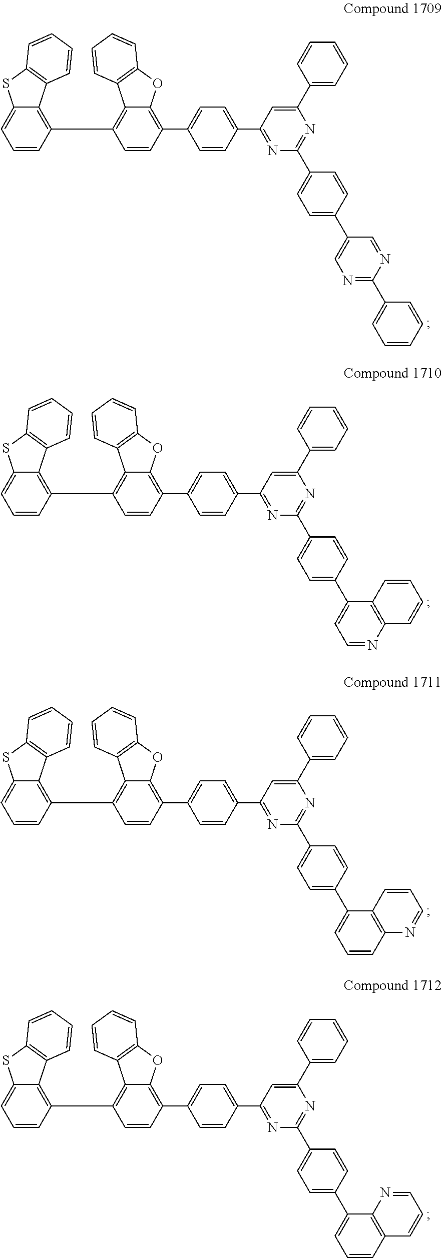 [SMILES: c1ccc(-c2cc(-c3ccc(-c4ccc(-c5cccc6sc7ccccc7c56)c5c4oc4ccccc45)cc3)nc(-c3ccc(-c4cnc(-c5ccccc5)nc4)cc3)n2)cc1 ; c1ccc(-c2cc(-c3ccc(-c4ccc(-c5cccc6sc7ccccc7c56)c5c4oc4ccccc45)cc3)nc(-c3ccc(-c4ccnc5ccccc45)cc3)n2)cc1 ; c1ccc(-c2cc(-c3ccc(-c4ccc(-c5cccc6sc7ccccc7c56)c5c4oc4ccccc45)cc3)nc(-c3ccc(-c4cccc5ncccc45)cc3)n2)cc1 ; c1ccc(-c2cc(-c3ccc(-c4ccc(-c5cccc6sc7ccccc7c56)c5c4oc4ccccc45)cc3)nc(-c3ccc(-c4cccc5cccnc45)cc3)n2)cc1]

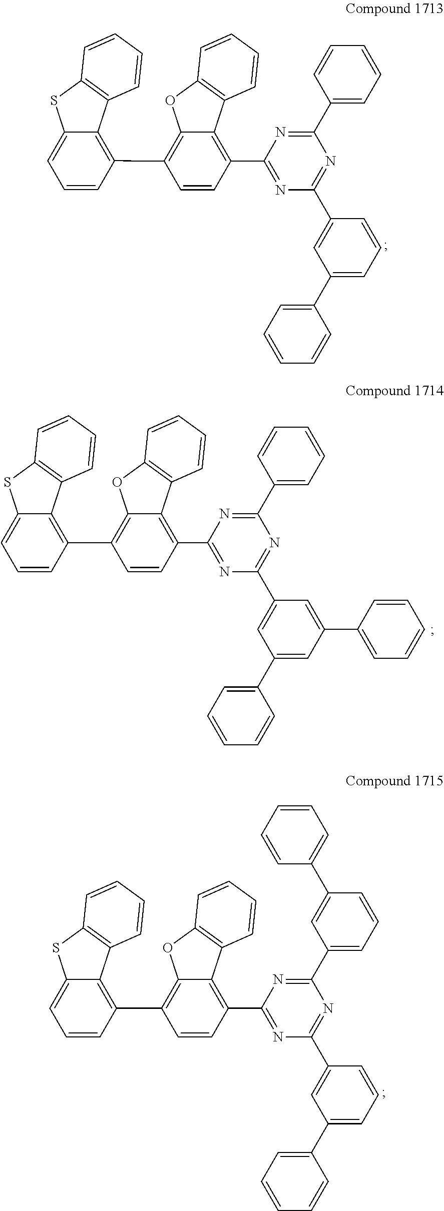 [SMILES: c1ccc(-c2cccc(-c3nc(-c4ccccc4)nc(-c4ccc(-c5cccc6sc7ccccc7c56)c5oc6ccccc6c45)n3)c2)cc1 ; c1ccc(-c2cc(-c3ccccc3)cc(-c3nc(-c4ccccc4)nc(-c4ccc(-c5cccc6sc7ccccc7c56)c5oc6ccccc6c45)n3)c2)cc1 ; c1ccc(-c2cccc(-c3nc(-c4cccc(-c5ccccc5)c4)nc(-c4ccc(-c5cccc6sc7ccccc7c56)c5oc6ccccc6c45)n3)c2)cc1]

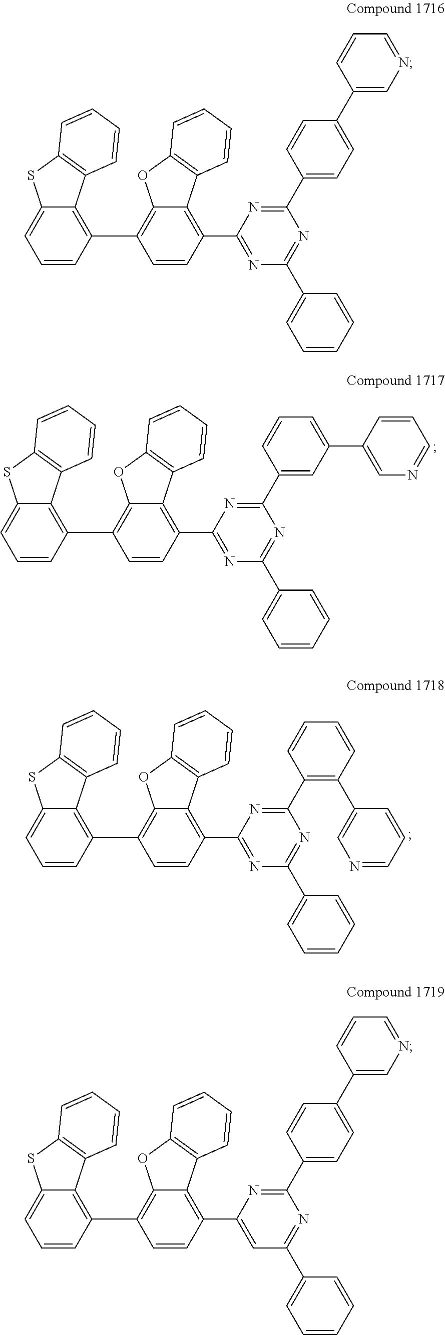 [SMILES: c1ccc(-c2nc(-c3ccc(-c4cccnc4)cc3)nc(-c3ccc(-c4cccc5sc6ccccc6c45)c4oc5ccccc5c34)n2)cc1 ; c1ccc(-c2nc(-c3cccc(-c4cccnc4)c3)nc(-c3ccc(-c4cccc5sc6ccccc6c45)c4oc5ccccc5c34)n2)cc1 ; c1ccc(-c2nc(-c3ccccc3-c3cccnc3)nc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3ccc(-c4cccnc4)cc3)n2)cc1]

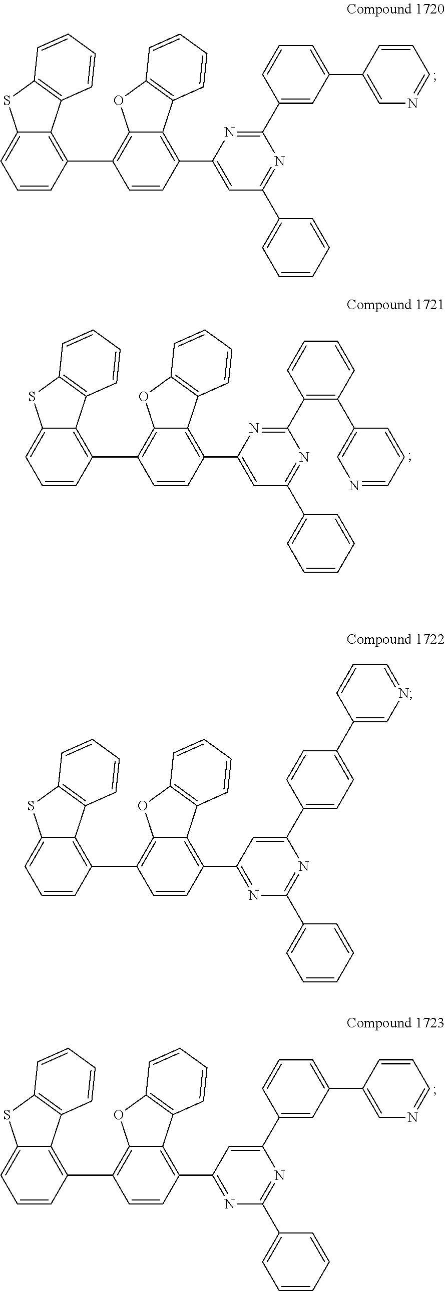 [SMILES: c1ccc(-c2cc(-c3ccc(-c4cccc5sc6ccccc6c45)c4oc5ccccc5c34)nc(-c3cccc(-c4cccnc4)c3)n2)cc1 ; c1ccc(-c2cc(-c3ccc(-c4cccc5sc6ccccc6c45)c4oc5ccccc5c34)nc(-c3ccccc3-c3cccnc3)n2)cc1 ; c1ccc(-c2nc(-c3ccc(-c4cccnc4)cc3)cc(-c3ccc(-c4cccc5sc6ccccc6c45)c4oc5ccccc5c34)n2)cc1 ; c1ccc(-c2nc(-c3cccc(-c4cccnc4)c3)cc(-c3ccc(-c4cccc5sc6ccccc6c45)c4oc5ccccc5c34)n2)cc1]

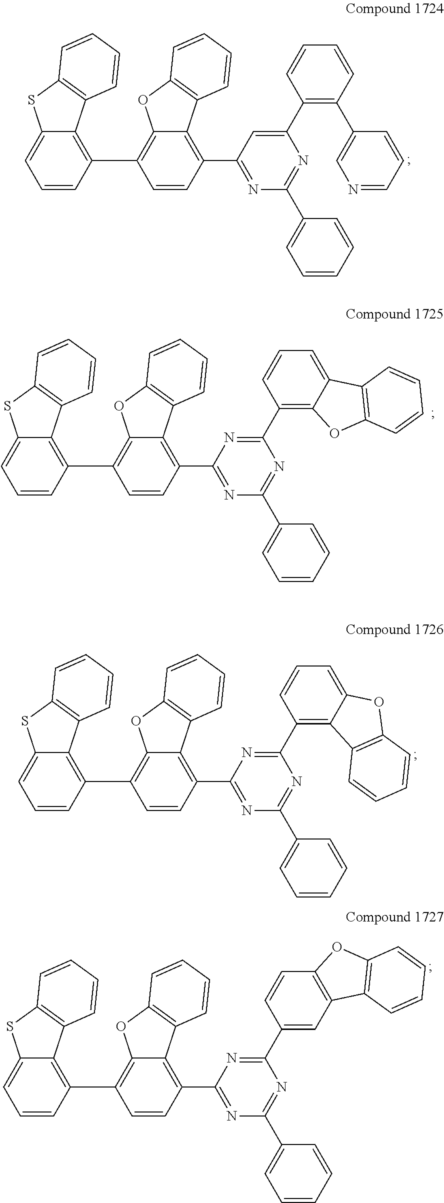 [SMILES: c1ccc(-c2nc(-c3ccccc3-c3cccnc3)cc(-c3ccc(-c4cccc5sc6ccccc6c45)c4oc5ccccc5c34)n2)cc1 ; c1ccc(-c2nc(-c3cccc4c3oc3ccccc34)nc(-c3ccc(-c4cccc5sc6ccccc6c45)c4oc5ccccc5c34)n2)cc1 ; c1ccc(-c2nc(-c3cccc4oc5ccccc5c34)nc(-c3ccc(-c4cccc5sc6ccccc6c45)c4oc5ccccc5c34)n2)cc1 ; c1ccc(-c2nc(-c3ccc4oc5ccccc5c4c3)nc(-c3ccc(-c4cccc5sc6ccccc6c45)c4oc5ccccc5c34)n2)cc1]

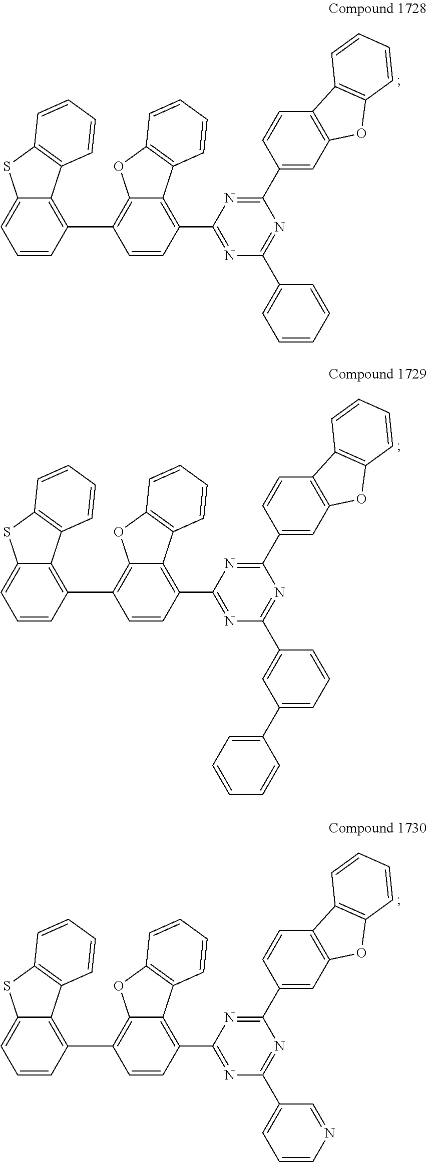 [SMILES: c1ccc(-c2nc(-c3ccc4c(c3)oc3ccccc34)nc(-c3ccc(-c4cccc5sc6ccccc6c45)c4oc5ccccc5c34)n2)cc1 ; c1ccc(-c2cccc(-c3nc(-c4ccc5c(c4)oc4ccccc45)nc(-c4ccc(-c5cccc6sc7ccccc7c56)c5oc6ccccc6c45)n3)c2)cc1 ; c1cncc(-c2nc(-c3ccc4c(c3)oc3ccccc34)nc(-c3ccc(-c4cccc5sc6ccccc6c45)c4oc5ccccc5c34)n2)c1]

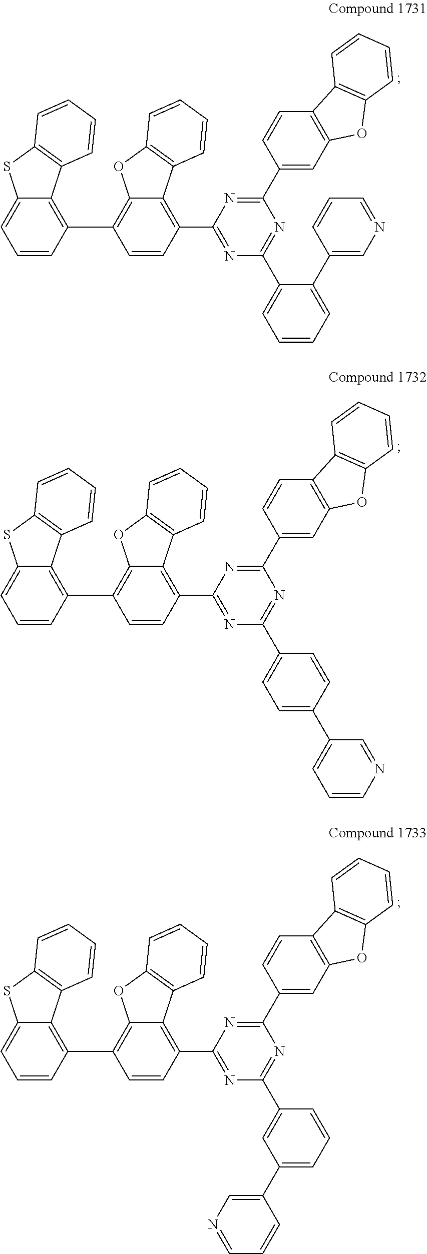 [SMILES: c1cncc(-c2ccccc2-c2nc(-c3ccc4c(c3)oc3ccccc34)nc(-c3ccc(-c4cccc5sc6ccccc6c45)c4oc5ccccc5c34)n2)c1 ; c1cncc(-c2ccc(-c3nc(-c4ccc5c(c4)oc4ccccc45)nc(-c4ccc(-c5cccc6sc7ccccc7c56)c5oc6ccccc6c45)n3)cc2)c1 ; c1cncc(-c2cccc(-c3nc(-c4ccc5c(c4)oc4ccccc45)nc(-c4ccc(-c5cccc6sc7ccccc7c56)c5oc6ccccc6c45)n3)c2)c1]

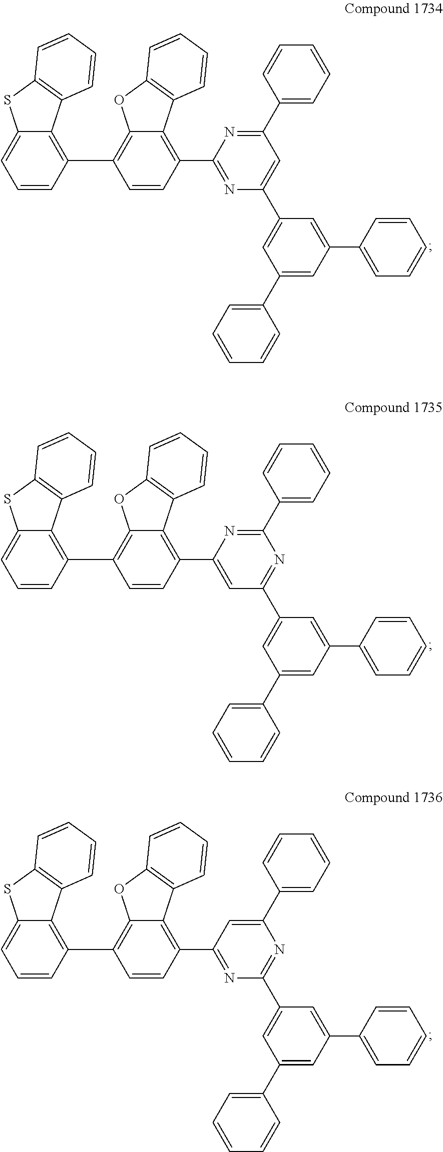 [SMILES: c1ccc(-c2cc(-c3ccccc3)cc(-c3cc(-c4ccccc4)nc(-c4ccc(-c5cccc6sc7ccccc7c56)c5oc6ccccc6c45)n3)c2)cc1 ; c1ccc(-c2cc(-c3ccccc3)cc(-c3cc(-c4ccc(-c5cccc6sc7ccccc7c56)c5oc6ccccc6c45)nc(-c4ccccc4)n3)c2)cc1 ; c1ccc(-c2cc(-c3ccccc3)cc(-c3nc(-c4ccccc4)cc(-c4ccc(-c5cccc6sc7ccccc7c56)c5oc6ccccc6c45)n3)c2)cc1]

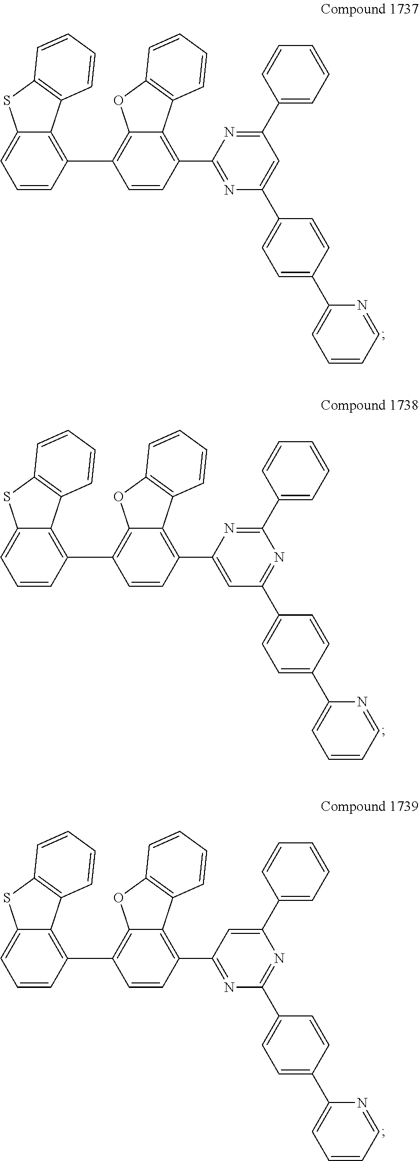 [SMILES: c1ccc(-c2cc(-c3ccc(-c4ccccn4)cc3)nc(-c3ccc(-c4cccc5sc6ccccc6c45)c4oc5ccccc5c34)n2)cc1 ; c1ccc(-c2nc(-c3ccc(-c4ccccn4)cc3)cc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3ccc(-c4ccccn4)cc3)n2)cc1]

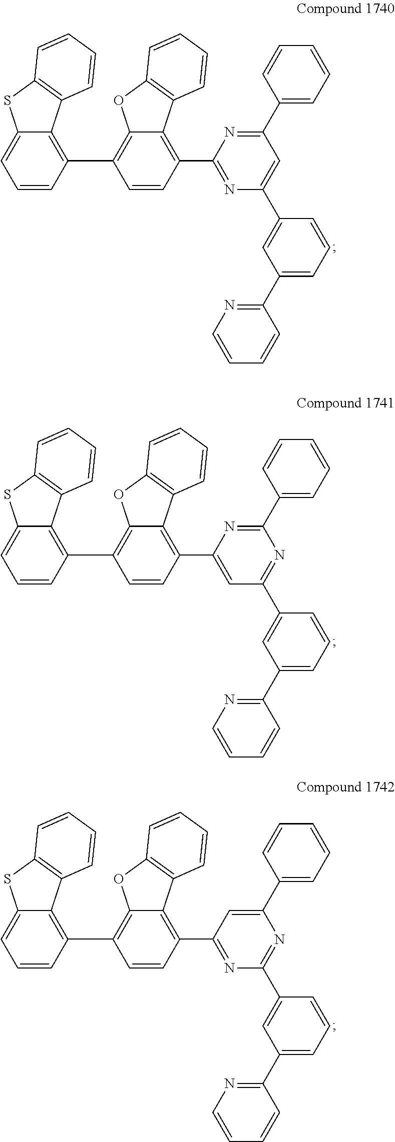 [SMILES: c1ccc(-c2cc(-c3cccc(-c4ccccn4)c3)nc(-c3ccc(-c4cccc5sc6ccccc6c45)c4oc5ccccc5c34)n2)cc1 ; c1ccc(-c2nc(-c3cccc(-c4ccccn4)c3)cc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3cccc(-c4ccccn4)c3)n2)cc1]

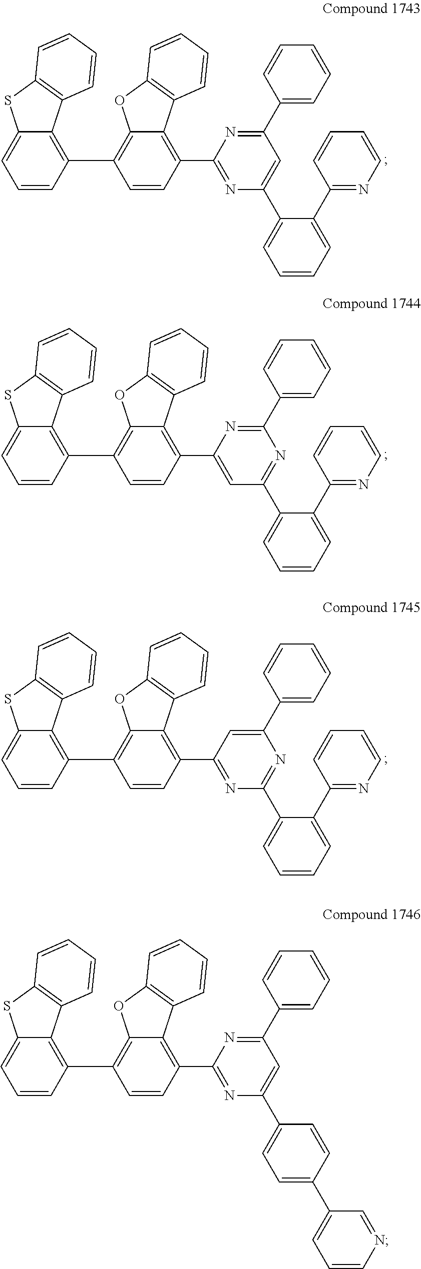 [SMILES: c1ccc(-c2cc(-c3ccccc3-c3ccccn3)nc(-c3ccc(-c4cccc5sc6ccccc6c45)c4oc5ccccc5c34)n2)cc1 ; c1ccc(-c2nc(-c3ccccc3-c3ccccn3)cc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3ccccc3-c3ccccn3)n2)cc1 ; c1ccc(-c2cc(-c3ccc(-c4cccnc4)cc3)nc(-c3ccc(-c4cccc5sc6ccccc6c45)c4oc5ccccc5c34)n2)cc1]

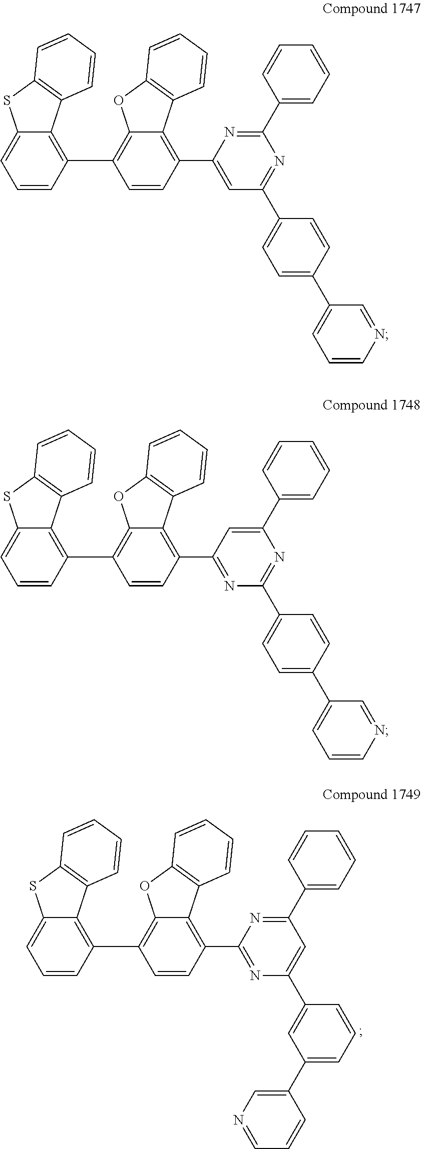 [SMILES: c1ccc(-c2nc(-c3ccc(-c4cccnc4)cc3)cc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3ccc(-c4cccnc4)cc3)n2)cc1 ; c1ccc(-c2cc(-c3cccc(-c4cccnc4)c3)nc(-c3ccc(-c4cccc5sc6ccccc6c45)c4oc5ccccc5c34)n2)cc1]

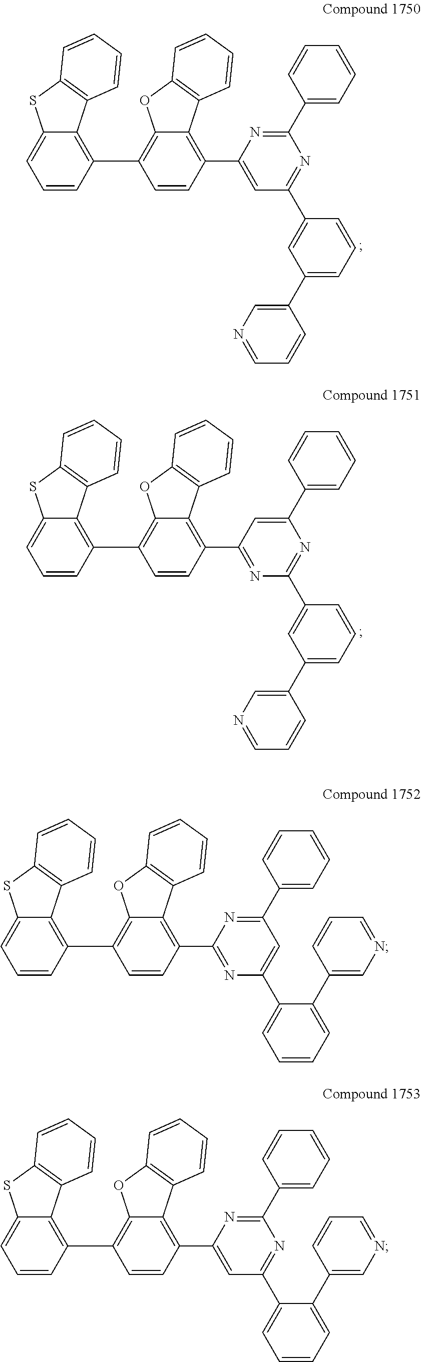 [SMILES: c1ccc(-c2nc(-c3cccc(-c4cccnc4)c3)cc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3cccc(-c4cccnc4)c3)n2)cc1 ; c1ccc(-c2cc(-c3ccccc3-c3cccnc3)nc(-c3ccc(-c4cccc5sc6ccccc6c45)c4oc5ccccc5c34)n2)cc1 ; c1ccc(-c2nc(-c3ccccc3-c3cccnc3)cc(-c3ccc(-c4cccc5sc6ccccc6c45)c4oc5ccccc5c34)n2)cc1]

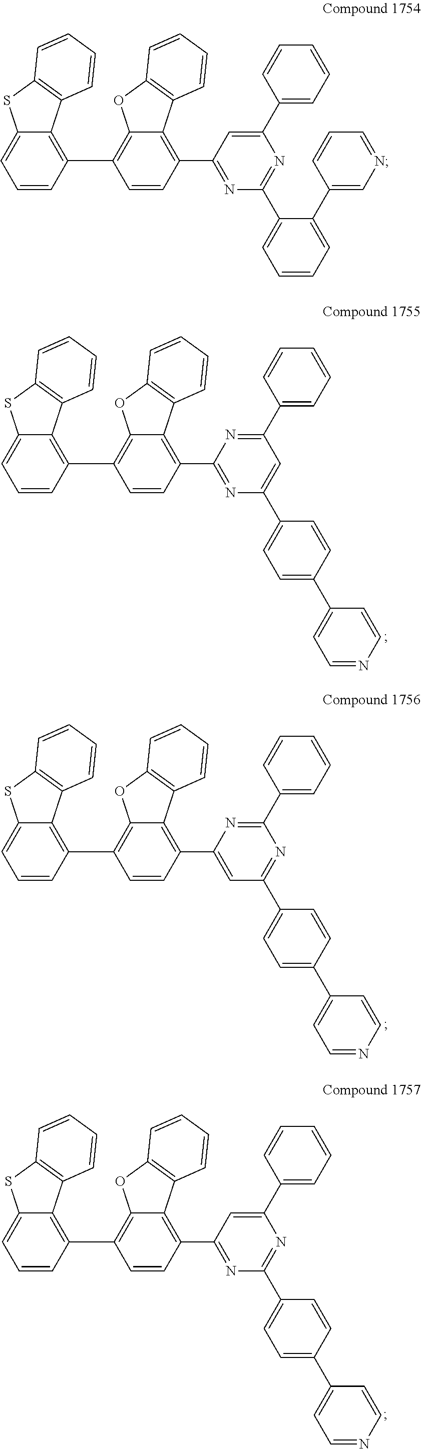 [SMILES: c1ccc(-c2cc(-c3ccc(-c4cccc5sc6ccccc6c45)c4oc5ccccc5c34)nc(-c3ccccc3-c3cccnc3)n2)cc1 ; c1ccc(-c2cc(-c3ccc(-c4ccncc4)cc3)nc(-c3ccc(-c4cccc5sc6ccccc6c45)c4oc5ccccc5c34)n2)cc1 ; c1ccc(-c2nc(-c3ccc(-c4ccncc4)cc3)cc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3ccc(-c4ccncc4)cc3)n2)cc1]

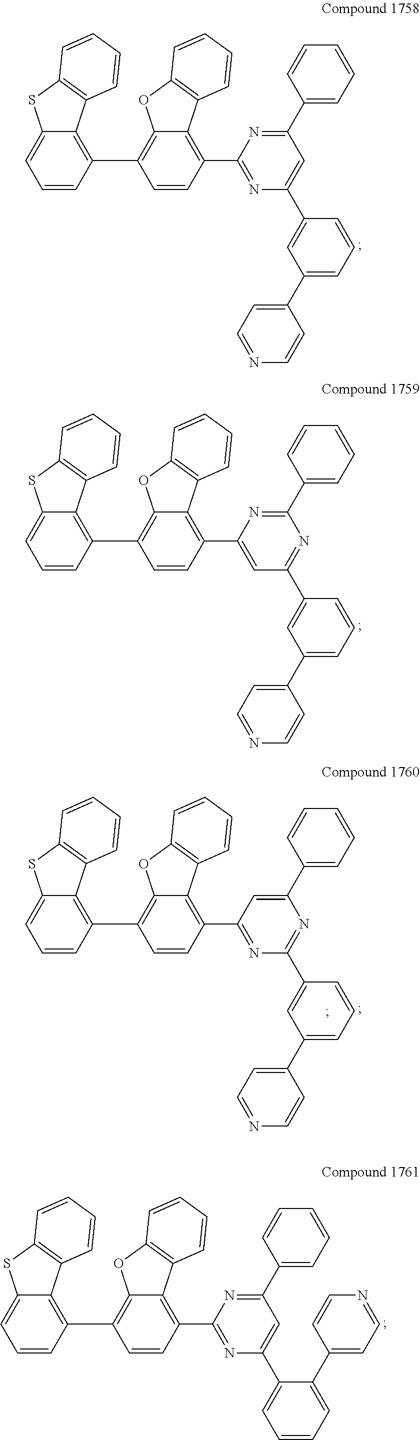 [SMILES: c1ccc(-c2cc(-c3cccc(-c4ccncc4)c3)nc(-c3ccc(-c4cccc5sc6ccccc6c45)c4oc5ccccc5c34)n2)cc1 ; c1ccc(-c2nc(-c3cccc(-c4ccncc4)c3)cc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3cccc(-c4ccncc4)c3)n2)cc1 ; c1ccc(-c2cc(-c3ccccc3-c3ccncc3)nc(-c3ccc(-c4cccc5sc6ccccc6c45)c4oc5ccccc5c34)n2)cc1]

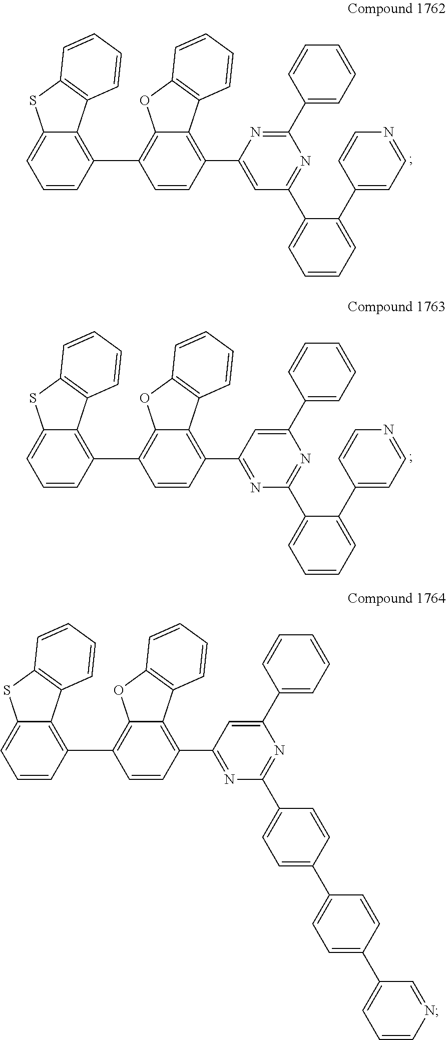 [SMILES: c1ccc(-c2nc(-c3ccccc3-c3ccncc3)cc(-c3ccc(-c4cccc5sc6ccccc6c45)c4oc5ccccc5c34)n2)cc1 ; c1ccc(-c2cc(-c3ccc(-c4cccc5sc6ccccc6c45)c4oc5ccccc5c34)nc(-c3ccccc3-c3ccncc3)n2)cc1 ; c1ccc(-c2cc(-c3ccc(-c4cccc5sc6ccccc6c45)c4oc5ccccc5c34)nc(-c3ccc(-c4ccc(-c5cccnc5)cc4)cc3)n2)cc1]

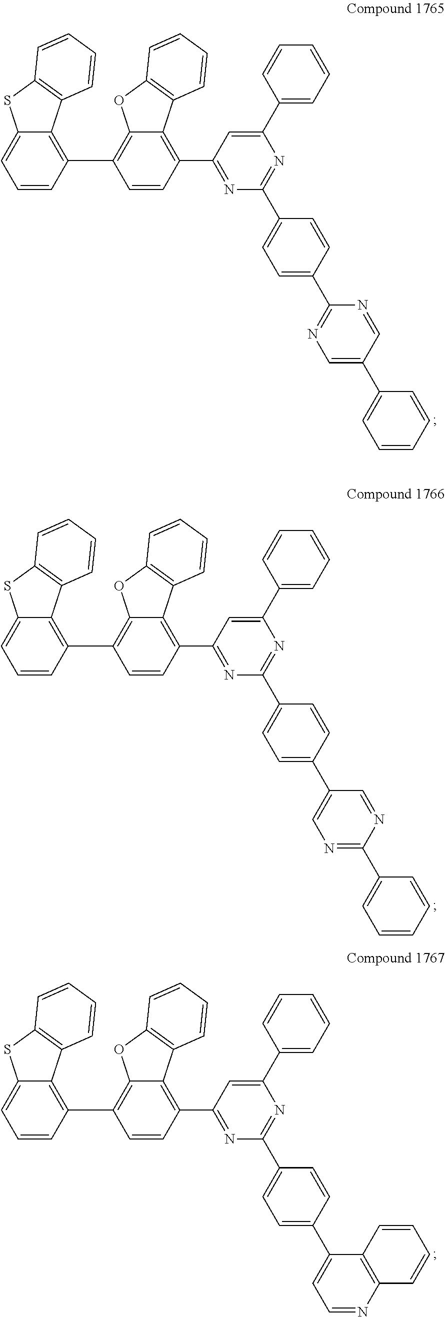 [SMILES: c1ccc(-c2cnc(-c3ccc(-c4nc(-c5ccccc5)cc(-c5ccc(-c6cccc7sc8ccccc8c67)c6oc7ccccc7c56)n4)cc3)nc2)cc1 ; c1ccc(-c2cc(-c3ccc(-c4cccc5sc6ccccc6c45)c4oc5ccccc5c34)nc(-c3ccc(-c4cnc(-c5ccccc5)nc4)cc3)n2)cc1 ; c1ccc(-c2cc(-c3ccc(-c4cccc5sc6ccccc6c45)c4oc5ccccc5c34)nc(-c3ccc(-c4ccnc5ccccc45)cc3)n2)cc1]

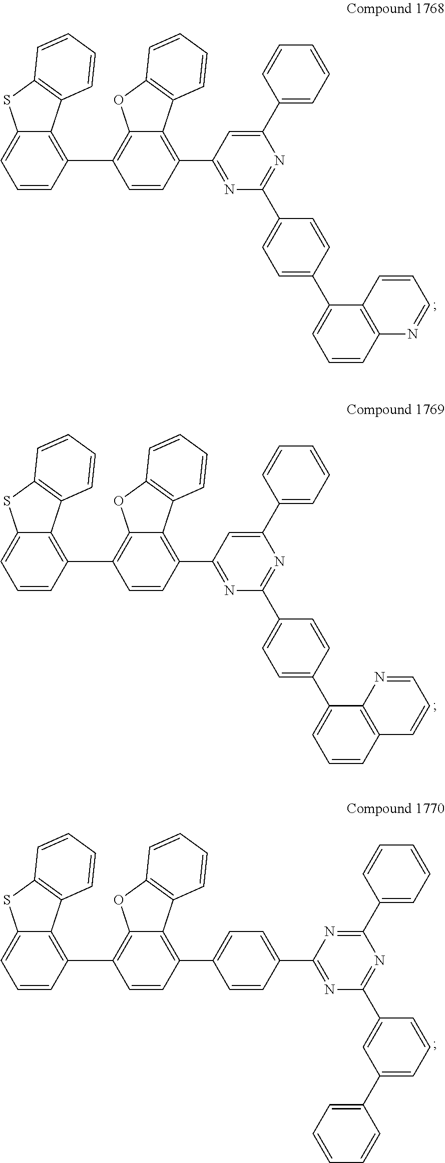 [SMILES: c1ccc(-c2cc(-c3ccc(-c4cccc5sc6ccccc6c45)c4oc5ccccc5c34)nc(-c3ccc(-c4cccc5ncccc45)cc3)n2)cc1 ; c1ccc(-c2cc(-c3ccc(-c4cccc5sc6ccccc6c45)c4oc5ccccc5c34)nc(-c3ccc(-c4cccc5cccnc45)cc3)n2)cc1 ; c1ccc(-c2cccc(-c3nc(-c4ccccc4)nc(-c4ccc(-c5ccc(-c6cccc7sc8ccccc8c67)c6oc7ccccc7c56)cc4)n3)c2)cc1]

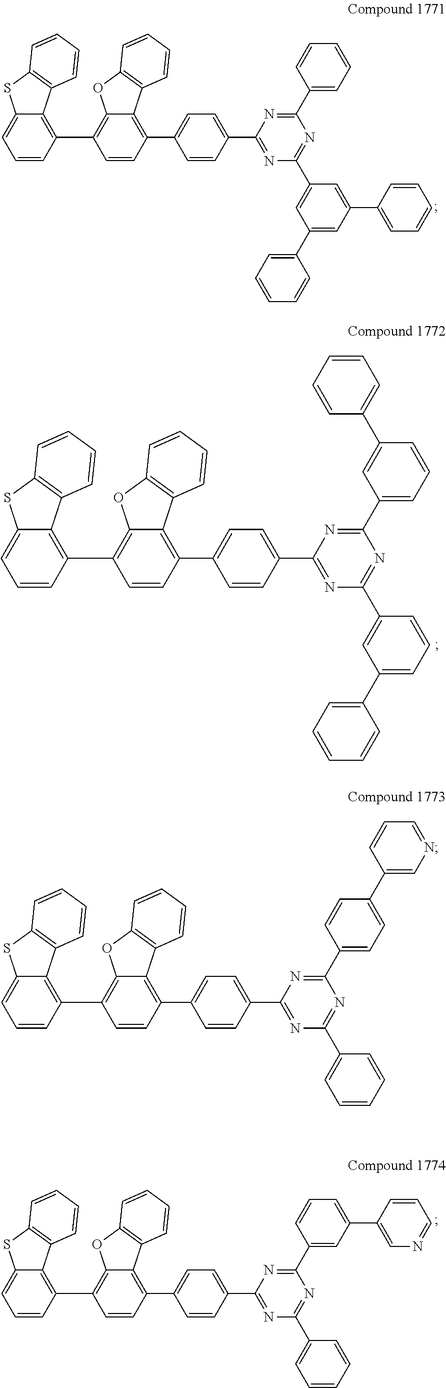 [SMILES: c1ccc(-c2cc(-c3ccccc3)cc(-c3nc(-c4ccccc4)nc(-c4ccc(-c5ccc(-c6cccc7sc8ccccc8c67)c6oc7ccccc7c56)cc4)n3)c2)cc1 ; c1ccc(-c2cccc(-c3nc(-c4ccc(-c5ccc(-c6cccc7sc8ccccc8c67)c6oc7ccccc7c56)cc4)nc(-c4cccc(-c5ccccc5)c4)n3)c2)cc1 ; c1ccc(-c2nc(-c3ccc(-c4cccnc4)cc3)nc(-c3ccc(-c4ccc(-c5cccc6sc7ccccc7c56)c5oc6ccccc6c45)cc3)n2)cc1 ; c1ccc(-c2nc(-c3ccc(-c4ccc(-c5cccc6sc7ccccc7c56)c5oc6ccccc6c45)cc3)nc(-c3cccc(-c4cccnc4)c3)n2)cc1]

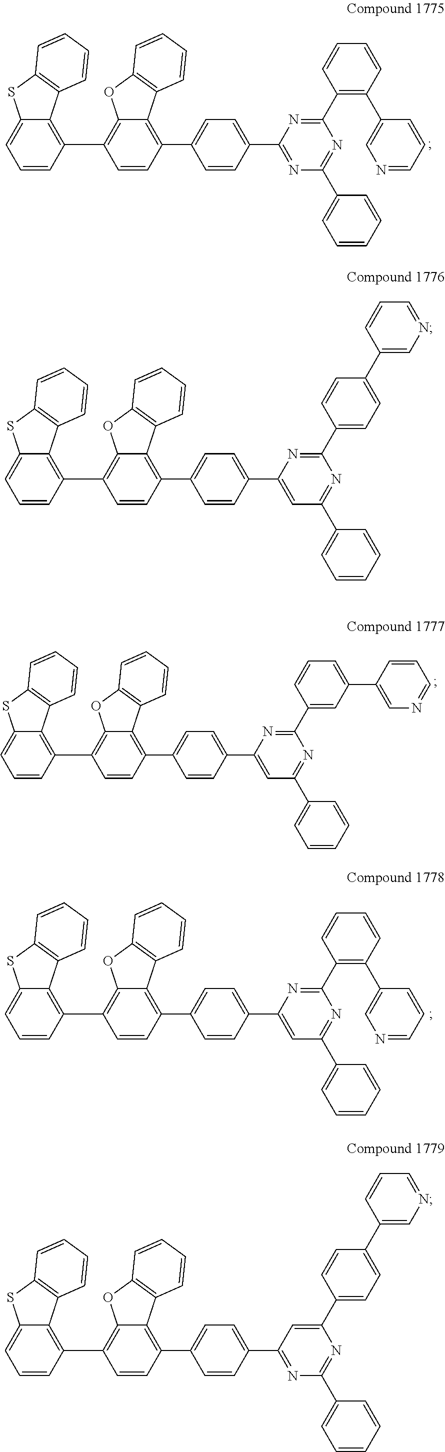 [SMILES: c1ccc(-c2nc(-c3ccc(-c4ccc(-c5cccc6sc7ccccc7c56)c5oc6ccccc6c45)cc3)nc(-c3ccccc3-c3cccnc3)n2)cc1 ; c1ccc(-c2cc(-c3ccc(-c4ccc(-c5cccc6sc7ccccc7c56)c5oc6ccccc6c45)cc3)nc(-c3ccc(-c4cccnc4)cc3)n2)cc1 ; c1ccc(-c2cc(-c3ccc(-c4ccc(-c5cccc6sc7ccccc7c56)c5oc6ccccc6c45)cc3)nc(-c3cccc(-c4cccnc4)c3)n2)cc1 ; c1ccc(-c2cc(-c3ccc(-c4ccc(-c5cccc6sc7ccccc7c56)c5oc6ccccc6c45)cc3)nc(-c3ccccc3-c3cccnc3)n2)cc1 ; c1ccc(-c2nc(-c3ccc(-c4cccnc4)cc3)cc(-c3ccc(-c4ccc(-c5cccc6sc7ccccc7c56)c5oc6ccccc6c45)cc3)n2)cc1]

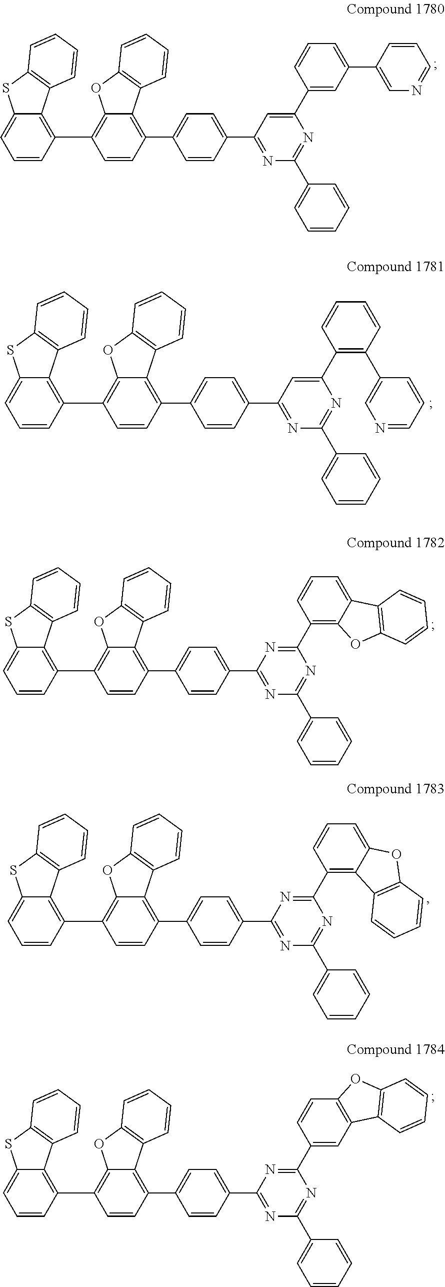 [SMILES: c1ccc(-c2nc(-c3ccc(-c4ccc(-c5cccc6sc7ccccc7c56)c5oc6ccccc6c45)cc3)cc(-c3cccc(-c4cccnc4)c3)n2)cc1 ; c1ccc(-c2nc(-c3ccc(-c4ccc(-c5cccc6sc7ccccc7c56)c5oc6ccccc6c45)cc3)cc(-c3ccccc3-c3cccnc3)n2)cc1 ; c1ccc(-c2nc(-c3ccc(-c4ccc(-c5cccc6sc7ccccc7c56)c5oc6ccccc6c45)cc3)nc(-c3cccc4c3oc3ccccc34)n2)cc1 ; c1ccc(-c2nc(-c3ccc(-c4ccc(-c5cccc6sc7ccccc7c56)c5oc6ccccc6c45)cc3)nc(-c3cccc4oc5ccccc5c34)n2)cc1 ; c1ccc(-c2nc(-c3ccc(-c4ccc(-c5cccc6sc7ccccc7c56)c5oc6ccccc6c45)cc3)nc(-c3ccc4oc5ccccc5c4c3)n2)cc1]

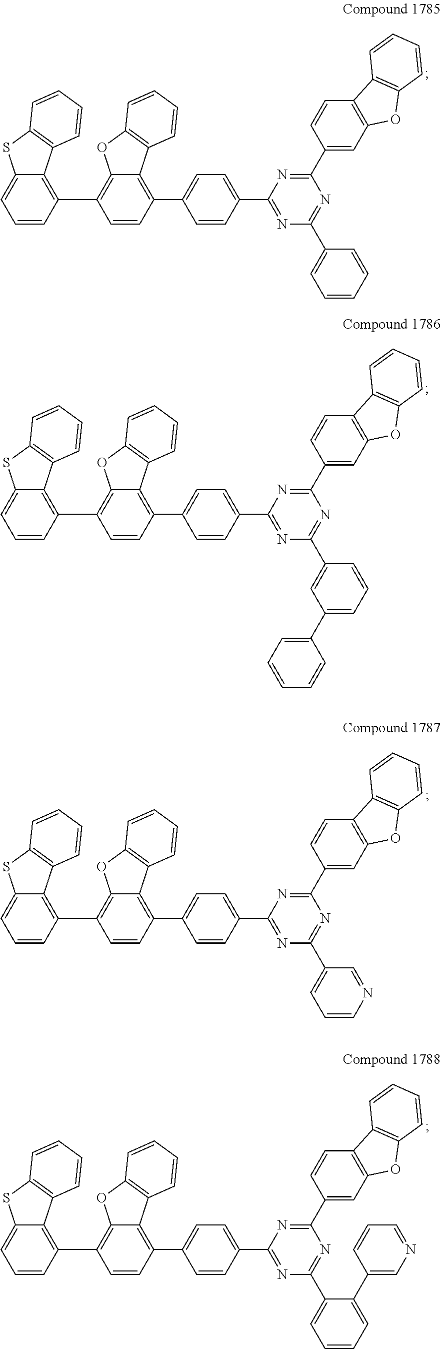 [SMILES: c1ccc(-c2nc(-c3ccc(-c4ccc(-c5cccc6sc7ccccc7c56)c5oc6ccccc6c45)cc3)nc(-c3ccc4c(c3)oc3ccccc34)n2)cc1 ; c1ccc(-c2cccc(-c3nc(-c4ccc(-c5ccc(-c6cccc7sc8ccccc8c67)c6oc7ccccc7c56)cc4)nc(-c4ccc5c(c4)oc4ccccc45)n3)c2)cc1 ; c1cncc(-c2nc(-c3ccc(-c4ccc(-c5cccc6sc7ccccc7c56)c5oc6ccccc6c45)cc3)nc(-c3ccc4c(c3)oc3ccccc34)n2)c1 ; c1cncc(-c2ccccc2-c2nc(-c3ccc(-c4ccc(-c5cccc6sc7ccccc7c56)c5oc6ccccc6c45)cc3)nc(-c3ccc4c(c3)oc3ccccc34)n2)c1]

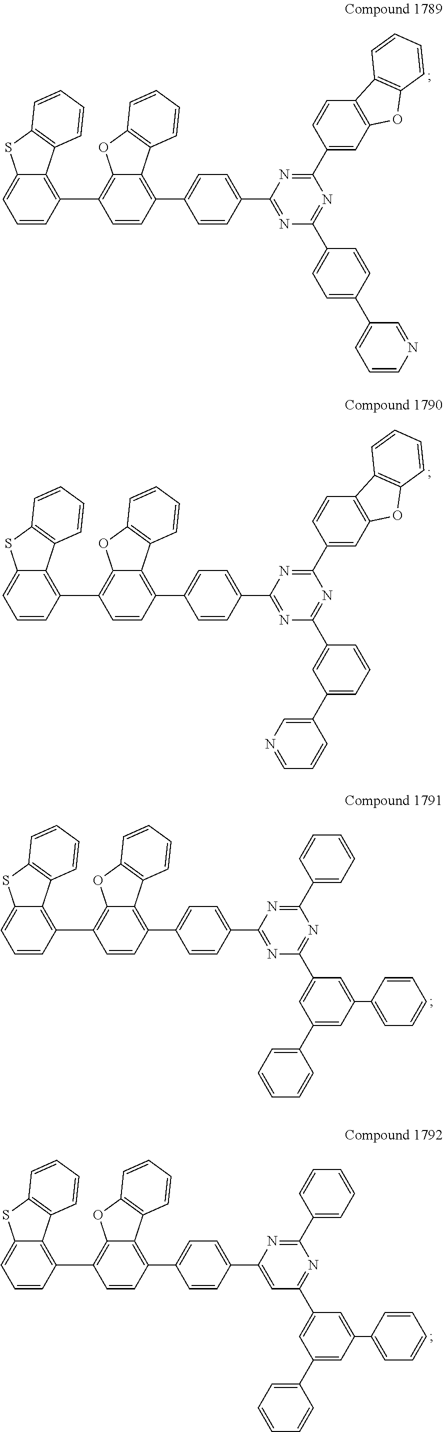 [SMILES: c1cncc(-c2ccc(-c3nc(-c4ccc(-c5ccc(-c6cccc7sc8ccccc8c67)c6oc7ccccc7c56)cc4)nc(-c4ccc5c(c4)oc4ccccc45)n3)cc2)c1 ; c1cncc(-c2cccc(-c3nc(-c4ccc(-c5ccc(-c6cccc7sc8ccccc8c67)c6oc7ccccc7c56)cc4)nc(-c4ccc5c(c4)oc4ccccc45)n3)c2)c1 ; c1ccc(-c2cc(-c3ccccc3)cc(-c3nc(-c4ccccc4)nc(-c4ccc(-c5ccc(-c6cccc7sc8ccccc8c67)c6oc7ccccc7c56)cc4)n3)c2)cc1 ; c1ccc(-c2cc(-c3ccccc3)cc(-c3cc(-c4ccc(-c5ccc(-c6cccc7sc8ccccc8c67)c6oc7ccccc7c56)cc4)nc(-c4ccccc4)n3)c2)cc1]

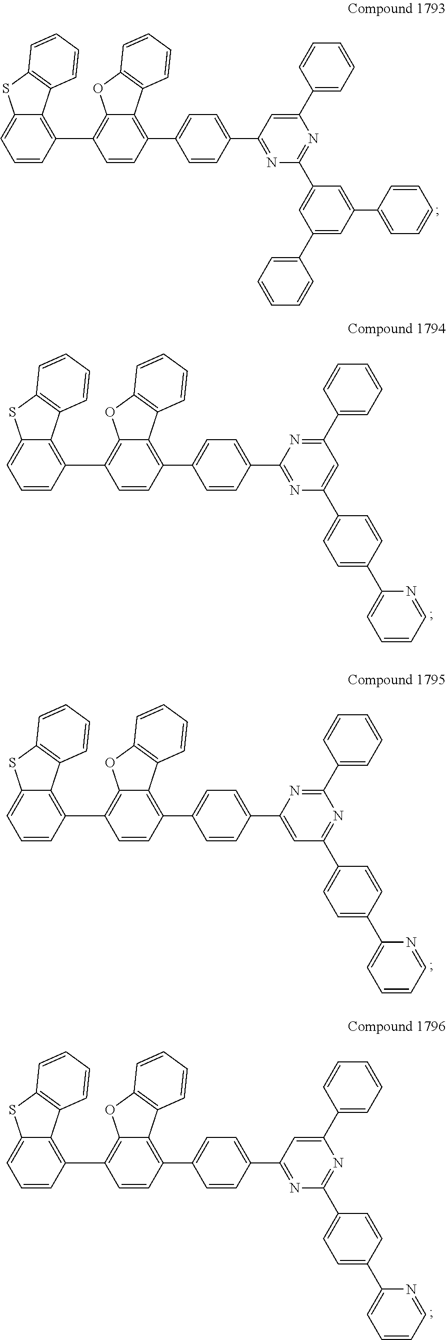 [SMILES: c1ccc(-c2cc(-c3ccccc3)cc(-c3nc(-c4ccccc4)cc(-c4ccc(-c5ccc(-c6cccc7sc8ccccc8c67)c6oc7ccccc7c56)cc4)n3)c2)cc1 ; c1ccc(-c2cc(-c3ccc(-c4ccccn4)cc3)nc(-c3ccc(-c4ccc(-c5cccc6sc7ccccc7c56)c5oc6ccccc6c45)cc3)n2)cc1 ; c1ccc(-c2nc(-c3ccc(-c4ccccn4)cc3)cc(-c3ccc(-c4ccc(-c5cccc6sc7ccccc7c56)c5oc6ccccc6c45)cc3)n2)cc1 ; c1ccc(-c2cc(-c3ccc(-c4ccc(-c5cccc6sc7ccccc7c56)c5oc6ccccc6c45)cc3)nc(-c3ccc(-c4ccccn4)cc3)n2)cc1]

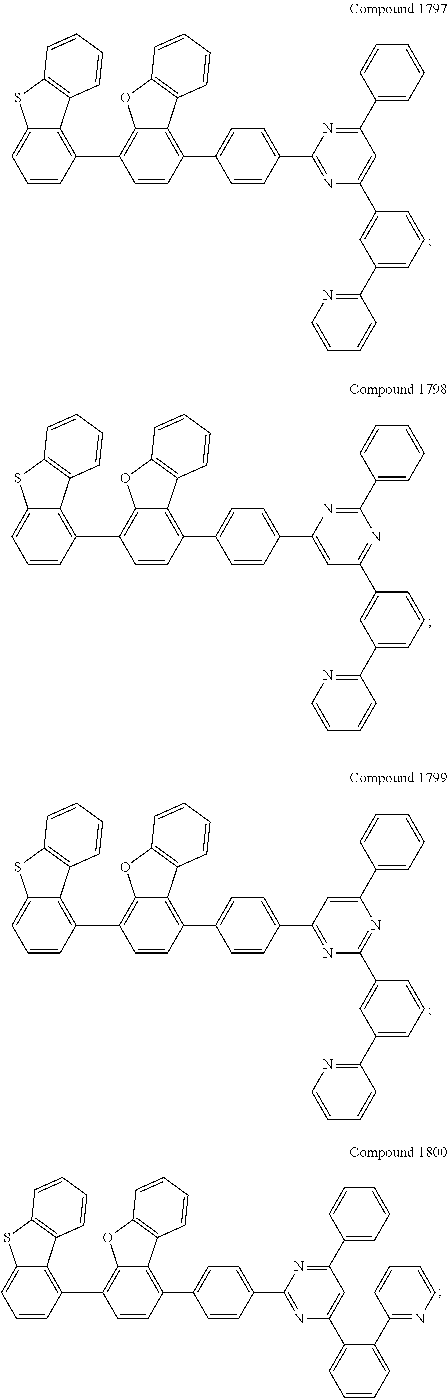 [SMILES: c1ccc(-c2cc(-c3cccc(-c4ccccn4)c3)nc(-c3ccc(-c4ccc(-c5cccc6sc7ccccc7c56)c5oc6ccccc6c45)cc3)n2)cc1 ; c1ccc(-c2nc(-c3ccc(-c4ccc(-c5cccc6sc7ccccc7c56)c5oc6ccccc6c45)cc3)cc(-c3cccc(-c4ccccn4)c3)n2)cc1 ; c1ccc(-c2cc(-c3ccc(-c4ccc(-c5cccc6sc7ccccc7c56)c5oc6ccccc6c45)cc3)nc(-c3cccc(-c4ccccn4)c3)n2)cc1 ; c1ccc(-c2cc(-c3ccccc3-c3ccccn3)nc(-c3ccc(-c4ccc(-c5cccc6sc7ccccc7c56)c5oc6ccccc6c45)cc3)n2)cc1]

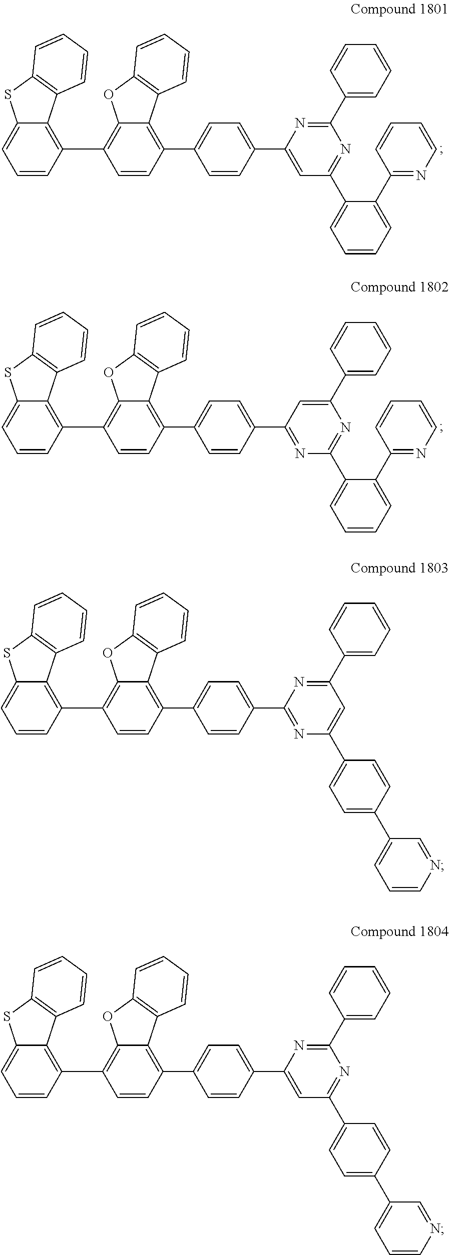 [SMILES: c1ccc(-c2nc(-c3ccc(-c4ccc(-c5cccc6sc7ccccc7c56)c5oc6ccccc6c45)cc3)cc(-c3ccccc3-c3ccccn3)n2)cc1 ; c1ccc(-c2cc(-c3ccc(-c4ccc(-c5cccc6sc7ccccc7c56)c5oc6ccccc6c45)cc3)nc(-c3ccccc3-c3ccccn3)n2)cc1 ; c1ccc(-c2cc(-c3ccc(-c4cccnc4)cc3)nc(-c3ccc(-c4ccc(-c5cccc6sc7ccccc7c56)c5oc6ccccc6c45)cc3)n2)cc1 ; c1ccc(-c2nc(-c3ccc(-c4cccnc4)cc3)cc(-c3ccc(-c4ccc(-c5cccc6sc7ccccc7c56)c5oc6ccccc6c45)cc3)n2)cc1]

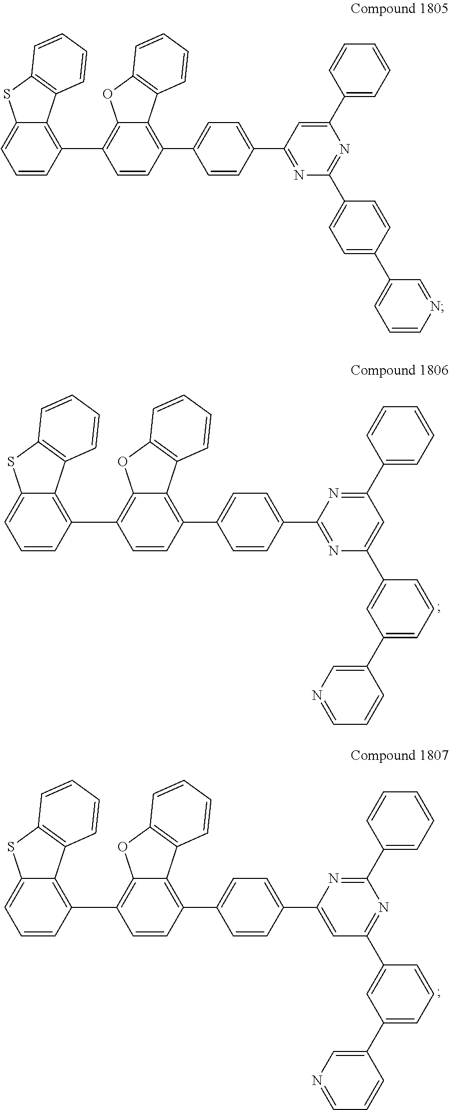 [SMILES: c1ccc(-c2cc(-c3ccc(-c4ccc(-c5cccc6sc7ccccc7c56)c5oc6ccccc6c45)cc3)nc(-c3ccc(-c4cccnc4)cc3)n2)cc1 ; c1ccc(-c2cc(-c3cccc(-c4cccnc4)c3)nc(-c3ccc(-c4ccc(-c5cccc6sc7ccccc7c56)c5oc6ccccc6c45)cc3)n2)cc1 ; c1ccc(-c2nc(-c3ccc(-c4ccc(-c5cccc6sc7ccccc7c56)c5oc6ccccc6c45)cc3)cc(-c3cccc(-c4cccnc4)c3)n2)cc1]

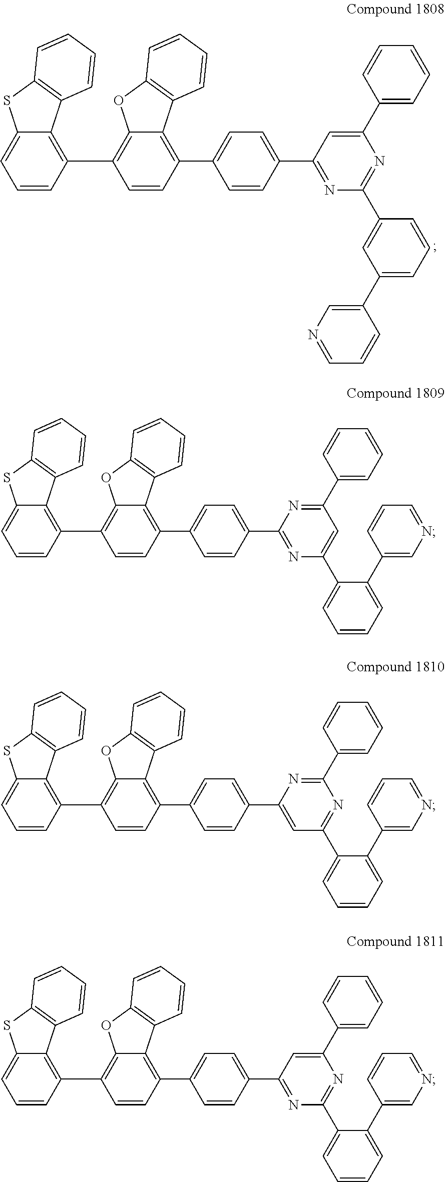 [SMILES: c1ccc(-c2cc(-c3ccc(-c4ccc(-c5cccc6sc7ccccc7c56)c5oc6ccccc6c45)cc3)nc(-c3cccc(-c4cccnc4)c3)n2)cc1 ; c1ccc(-c2cc(-c3ccccc3-c3cccnc3)nc(-c3ccc(-c4ccc(-c5cccc6sc7ccccc7c56)c5oc6ccccc6c45)cc3)n2)cc1 ; c1ccc(-c2nc(-c3ccc(-c4ccc(-c5cccc6sc7ccccc7c56)c5oc6ccccc6c45)cc3)cc(-c3ccccc3-c3cccnc3)n2)cc1 ; c1ccc(-c2cc(-c3ccc(-c4ccc(-c5cccc6sc7ccccc7c56)c5oc6ccccc6c45)cc3)nc(-c3ccccc3-c3cccnc3)n2)cc1]

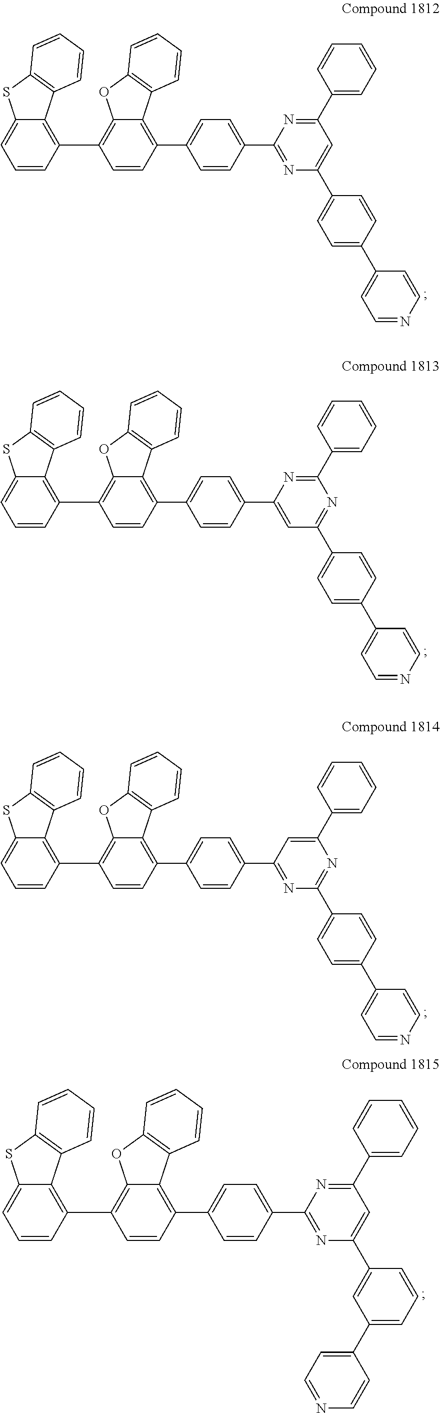 [SMILES: c1ccc(-c2cc(-c3ccc(-c4ccncc4)cc3)nc(-c3ccc(-c4ccc(-c5cccc6sc7ccccc7c56)c5oc6ccccc6c45)cc3)n2)cc1 ; c1ccc(-c2nc(-c3ccc(-c4ccncc4)cc3)cc(-c3ccc(-c4ccc(-c5cccc6sc7ccccc7c56)c5oc6ccccc6c45)cc3)n2)cc1 ; c1ccc(-c2cc(-c3ccc(-c4ccc(-c5cccc6sc7ccccc7c56)c5oc6ccccc6c45)cc3)nc(-c3ccc(-c4ccncc4)cc3)n2)cc1 ; c1ccc(-c2cc(-c3cccc(-c4ccncc4)c3)nc(-c3ccc(-c4ccc(-c5cccc6sc7ccccc7c56)c5oc6ccccc6c45)cc3)n2)cc1]

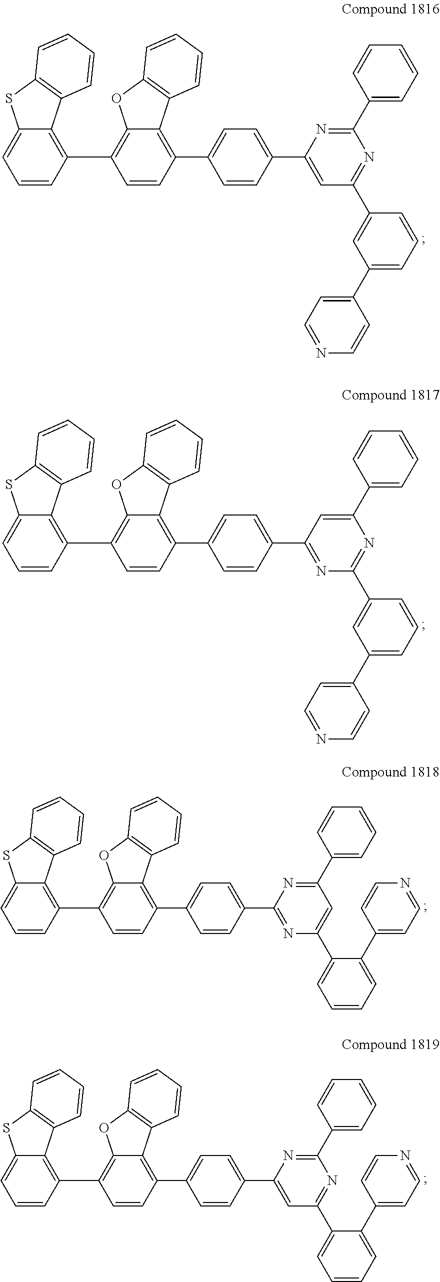 [SMILES: c1ccc(-c2nc(-c3ccc(-c4ccc(-c5cccc6sc7ccccc7c56)c5oc6ccccc6c45)cc3)cc(-c3cccc(-c4ccncc4)c3)n2)cc1 ; c1ccc(-c2cc(-c3ccc(-c4ccc(-c5cccc6sc7ccccc7c56)c5oc6ccccc6c45)cc3)nc(-c3cccc(-c4ccncc4)c3)n2)cc1 ; c1ccc(-c2cc(-c3ccccc3-c3ccncc3)nc(-c3ccc(-c4ccc(-c5cccc6sc7ccccc7c56)c5oc6ccccc6c45)cc3)n2)cc1 ; c1ccc(-c2nc(-c3ccc(-c4ccc(-c5cccc6sc7ccccc7c56)c5oc6ccccc6c45)cc3)cc(-c3ccccc3-c3ccncc3)n2)cc1]

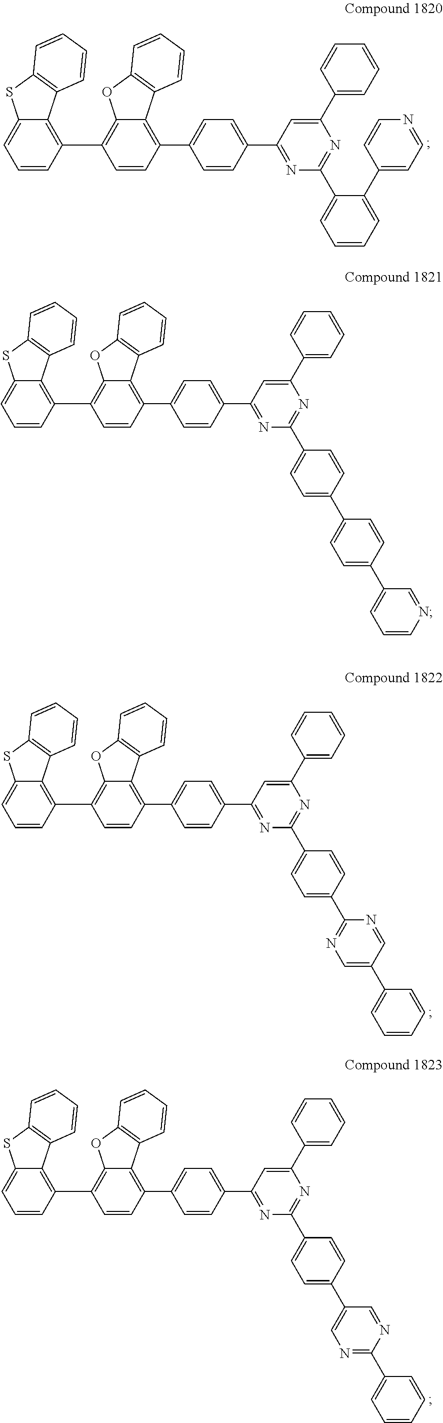 [SMILES: c1ccc(-c2cc(-c3ccc(-c4ccc(-c5cccc6sc7ccccc7c56)c5oc6ccccc6c45)cc3)nc(-c3ccccc3-c3ccncc3)n2)cc1 ; c1ccc(-c2cc(-c3ccc(-c4ccc(-c5cccc6sc7ccccc7c56)c5oc6ccccc6c45)cc3)nc(-c3ccc(-c4ccc(-c5cccnc5)cc4)cc3)n2)cc1 ; c1ccc(-c2cnc(-c3ccc(-c4nc(-c5ccccc5)cc(-c5ccc(-c6ccc(-c7cccc8sc9ccccc9c78)c7oc8ccccc8c67)cc5)n4)cc3)nc2)cc1 ; c1ccc(-c2cc(-c3ccc(-c4ccc(-c5cccc6sc7ccccc7c56)c5oc6ccccc6c45)cc3)nc(-c3ccc(-c4cnc(-c5ccccc5)nc4)cc3)n2)cc1]

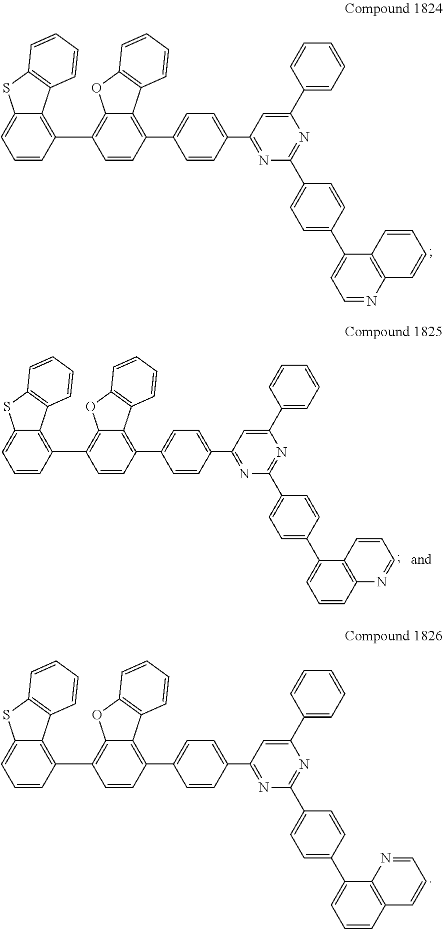 [SMILES: c1ccc(-c2cc(-c3ccc(-c4ccc(-c5cccc6sc7ccccc7c56)c5oc6ccccc6c45)cc3)nc(-c3ccc(-c4ccnc5ccccc45)cc3)n2)cc1 ; c1ccc(-c2cc(-c3ccc(-c4ccc(-c5cccc6sc7ccccc7c56)c5oc6ccccc6c45)cc3)nc(-c3ccc(-c4cccc5ncccc45)cc3)n2)cc1 ; c1ccc(-c2cc(-c3ccc(-c4ccc(-c5cccc6sc7ccccc7c56)c5oc6ccccc6c45)cc3)nc(-c3ccc(-c4cccc5cccnc45)cc3)n2)cc1]

The present invention also provides an organic electronic device, comprising a first electrode, a second electrode, and an organic layer disposed between the first electrode and the second electrode. The organic layer comprises the novel compound as described above. The novel compound may be, but is not limited to, any one of Compounds 1 to 1826.

Preferably, the organic electronic device is an organic light emitting device (OLED).

Specifically, the organic light emitting device may comprise:

a hole injection layer formed on the first electrode;

a hole transport layer formed on the hole injection layer;

an emission layer formed on the hole transport layer;

an electron transport layer formed on the emission layer;

an electron injection layer formed between the electron transport layer and the second electrode.

In one embodiment, the organic layer may be the electron transport layer, i.e., the electron transport layer comprises an electron transport material which is the novel compound as stated above.

For example, the electron transport layer may be a single-layered configuration or a multi-layered configuration disposed between the emission layer and the electron injection layer. When the electron transport layer is the multi-layered configuration, e.g., the electron transport layer comprises a first electron transport layer and a second electron transport layer, the first electron transport material of the first electron transport layer may be made of a single novel compound and the second electron transport material of the second electron transport layer may be made of another single novel compound or any single conventional compound. Or, the first electron transport material of the first electron transport layer may be made of a novel compound in combination with another single novel compound or any single conventional compound, and so as the second electron transport material.

Said first and/or second electron transport layer comprises the novel compound such as Compounds 1 to 1826. The OLEDs using the novel compound as the electron transport material can have a prolonged lifespan compared to the commercial OLEDs using known electron transport materials of ETL, such as BCP, TmPyPb, TPBi, 3TPYMB, BmPyPb, and DPyPA.

Preferably, the OLED further comprises a hole blocking layer (HBL), formed between the electron transport layer and the emission layer, to block holes overflow from the emission layer to the electron transport layer.

In another embodiment, the organic layer may be the hole blocking layer, i.e., the hole blocking layer comprises a hole blocking material which is the novel compound as stated above. More specifically, said hole blocking layer comprises the novel compound such as Compounds 1 to 1826. The OLEDs using the novel compound as the hole blocking material can have a prolonged lifespan compared to commercial OLEDs using known hole blocking materials of HBL, such as 2,9-dimethyl-4,7-diphenyl-1,10-phenanthroline (BCP) and 2,3,5,6-tetramethyl-phenyl-1,4-(bis-phthalimide) (TMPP).

Preferably, the hole injection layer may be a single-layered configuration or a multi-layered configuration, i.e., the OLED comprises a first hole injection layer and a second hole injection layer disposed between the first electrode and the hole transport layer.

The aforesaid hole injection layer(s) may be made of, for example, but not limited to: polyaniline, polyethylenedioxythiophene, 4,4′,4″-Tris[(3-methylphenyl)phenylamino]triphenylamine (m-MTDATA), or N¹,N^{1′}-(biphenyl-4,4′-diyl)bis(N¹-(naphthalen-1-yl)-N⁴,N^{4′}-diphenylbenzene-1,4-diamine).

Preferably, the hole transport layer may be a two-layered configuration, i.e., the OLED comprises a first hole transport layer and a second hole transport layer disposed between the two-layered hole injection layer and the emission layer.

Said first and second hole transport layers may be made of, for example, but not limited to: 1,1-bis[(di-4-tolylamino)phenylcyclohexane](TAPC), a carbazole derivative such as N-phenyl carbazole, and N⁴,N^{4′}-di(naphthalen-1-yl)-N⁴,N4′-diphenylbiphenyl-4,4′-diamine (NPB).

Preferably, the emission layer can be made of an emission material including a host and a dopant. The host of the emission material is, for example, but not limited to, 9-(4-(naphthalen-1-yl)phenyl)-10-(naphthalen-2-yl) anthracene.

For red OLEDs, the dopant of the emission material is, for example, but not limited to: organometallic compounds of iridium (II) having quinoline derivative ligands or isoquinoline derivative ligands; an osmium complex; or a platinum complex. For green OLEDs, the dopant of the emission material is, for example, but not limited to: diaminofluorenes; diaminoanthracenes; or organometallic compounds of iridium (II) having phenylpyridine ligands. For blue OLEDs, the dopant of the emission material is, for example, but not limited to: an aminoperylene derivative; a diaminochrysene; diaminopyrenes; or organicmetallic compounds of iridium (II) having pyridinato picolinate ligands. With various host materials of the emission layer, the OLED can emit lights in red, green or blue.

Preferably, the OLED comprises an electron blocking layer formed between the hole transport layer and the emission layer, to block electrons overflow from the emission layer to the hole transport layer. Said electron blocking layer may be made of 9,9′[1,1′-biphenyl]-4,4′-diylbis-9H-carbazole (CBP) or 4,4′,4″-tri(N-carbazolyl)-triphenylamine (TCTA), but it is not limited thereto.

In the presence of such a hole blocking layer and/or an electron blocking layer in an OLED, the OLED has an improved efficiency compared to a conventional OLED.

Said electron injection layer may be made of an electron injection material, for example, but not limited to (8-oxidonaphthalen-1-yl)lithium(II).

Said first electrode is, for example, but not limited to, an indium-doped tin oxide electrode.

Said second electrode has a work function lower than that of the first electrode. The second electrode is, for example, but not limited to, an aluminum electrode, an indium electrode, or a magnesium electrode.

Other objectives, advantages and novel features of the invention will become more apparent from the following detailed description when taken in conjunction with the accompanying drawings.

### BRIEF DESCRIPTION OF THE DRAWINGS

FIG. 1 illustrates a schematic side view of a conventional OLED.

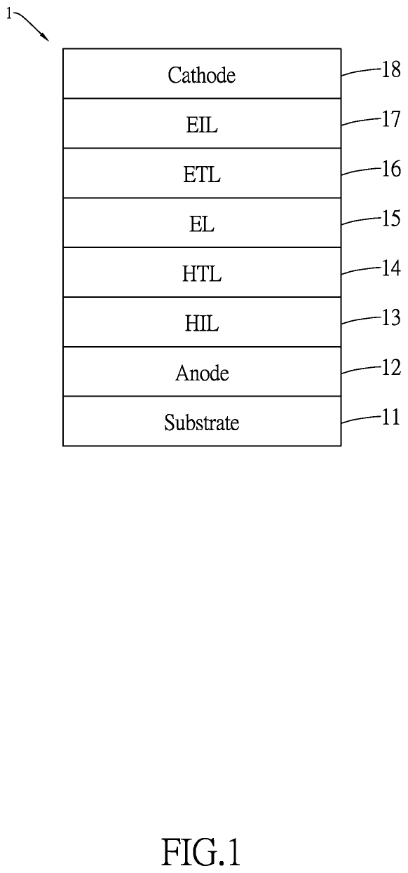

FIG. 2 illustrates a schematic side view of an OLED with a single electron transport layer.

FIG. 3 illustrates a schematic side view of an OLED with double electron transport layers.

### DETAILED DESCRIPTION OF THE PREFERRED EMBODIMENTS

Hereinafter, one skilled in the arts can easily realize the advantages and effects of a novel compound and an organic light emitting device using the same in accordance with the present invention from the following examples. It should be understood that the descriptions proposed herein are just preferable examples only for the purpose of illustrations, not intended to limit the scope of the invention. Various modifications and variations could be made in order to practice or apply the present invention without departing from the spirit and scope of the invention.

### Synthesis of Intermediate An

Intermediate An used for preparing a novel compound was synthesized by the following steps.

### Synthesis of Intermediate An-1

In step 1, the general synthesis pathway of Intermediate An-1 was summarized in Scheme A1, which can be used to prepare Intermediates A1 to A8.

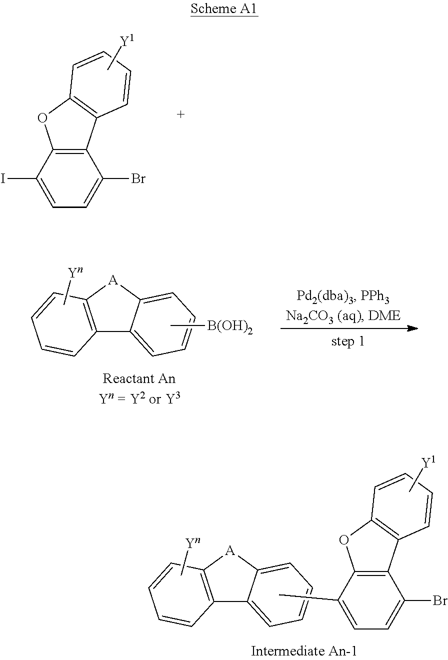

In Scheme A1, A is oxygen or sulfur; Y¹ to Y³ are each independently selected from the group consisting of: a hydrogen atom, a deuterium atom, an alkyl group having 1 to 12 carbon atoms, and an aryl group having 6 to 30 ring carbon atoms, and Y¹ to Y³ are the same or different.

### Synthesis of Intermediate A1-1

Taking Intermediate A1-1 as an example of Intermediate An-1, the synthesis pathway of Intermediate A1-1 was summarized in Scheme A1-1.

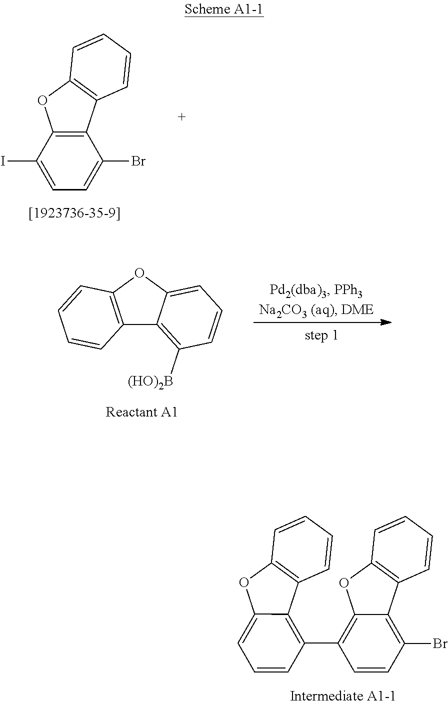 [SMILES: Brc1ccc(I)c2oc3ccccc3c12 ; OB(O)c1cccc2oc3ccccc3c12 ; Brc1ccc(-c2cccc3oc4ccccc4c23)c2oc3ccccc3c12]

A mixture of 1-bromo-4-iododibenzofuran (1.0 eq), 1-dibenzofuranboronic acid (1.0 eq), tris(dibenzylideneacetone)dipalladium[Pd₂(dba)₃] (0.005 eq), and triphenylphosphine (PPh₃) (0.02 eq) was in a mixed solution of methoxymethane (DME) (0.5 M) and Na₂CO₃ aqueous solution (2.0 M). The reaction mixture was heated to about 85° C. and stirred for 12 to 16 hours under nitrogen atmosphere. After the completion of the reaction, the reaction mixture was cooled to room temperature, and the precipitated crude product was then separated by filtration to obtain a crude product. After the filtration, the crude product was purified by recrystallization method using toluene to obtain a white solid product in 77.4% yield.

The white solid product was identified as Intermediate A1-1 by a field desorption mass spectroscopy (FD-MS) analysis. FD-MS analysis: C₂₄H₁₃BrO₂; theoretical value: 412.01; observed value: 412.01.

### Syntheses of Intermediates A2-1 to A8-1

Intermediates A2-1 to A8-1, which also can be used for preparing a novel compound, were respectively synthesized in a similar manner as Intermediate A1-1 through step 1, except that the starting material Reactant A1 was replaced by Reactants A2 to A8, respectively. All intermediates were analyzed as described above, and the results were listed in Table 1.

TABLE 1
The chemical structures and CAS No. of Reactant An used for
preparing Intermediates A1-1 to A8-1, and the chemical structures, yields,
formulae, and mass analyzed by FD-MS of Intermediates A1-1 to A8-1.

| Chemical Structure and | Chemical Structure of | Yield | Formula/ |
|---|---|---|---|
| CAS No. of Reactant An | Intermediate An-1 | (%) | Mass (M⁺) |
| 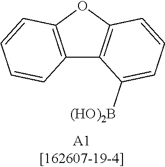 | 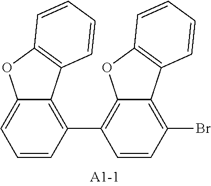 [SMILES: Brc1ccc(-c2cccc3oc4ccccc4c23)c2oc3ccccc3c12] | 77.4 | C₂₄H₁₃BrO₂/ 412.01 |
| 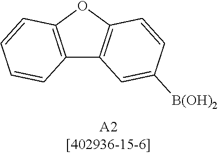 | 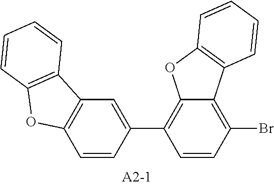 [SMILES: Brc1ccc(-c2ccc3oc4ccccc4c3c2)c2oc3ccccc3c12] | 83.5 | C₂₄H₁₃BrO₂/ 412.01 |
| 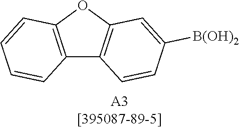 | 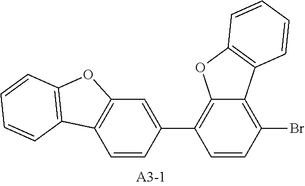 [SMILES: Brc1ccc(-c2ccc3c(c2)oc2ccccc23)c2oc3ccccc3c12] | 78.2 | C₂₄H₁₃BrO₂/ 412.01 |
| 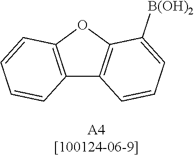 | 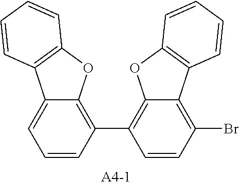 [SMILES: Brc1ccc(-c2cccc3c2oc2ccccc23)c2oc3ccccc3c12] | 81.9 | C₂₄H₁₃BrO₂/ 412.01 |
| 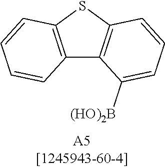 | 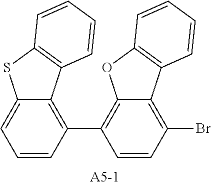 [SMILES: Brc1ccc(-c2cccc3sc4ccccc4c23)c2oc3ccccc3c12] | 75.0 | C₂₄H₁₃BrOS/ 427.99 |
| 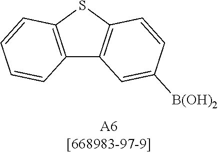 | 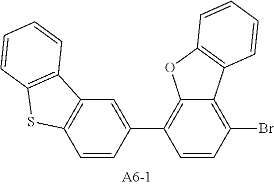 [SMILES: Brc1ccc(-c2ccc3sc4ccccc4c3c2)c2oc3ccccc3c12] | 77.5 | C₂₄H₁₃BrOS/ 427.99 |
| 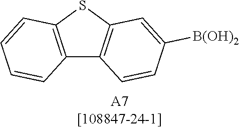 | 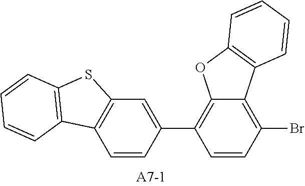 [SMILES: Brc1ccc(-c2ccc3c(c2)sc2ccccc23)c2oc3ccccc3c12] | 80.7 | C₂₄H₁₃BrOS/ 427.99 |
| 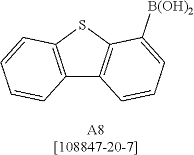 | 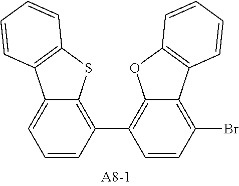 [SMILES: Brc1ccc(-c2cccc3c2sc2ccccc23)c2oc3ccccc3c12] | 83.0 | C₂₄H₁₃BrOS/ 427.99 |

Modifications of Intermediates A1-1 to A8-1

In addition to Intermediates A1-1 to A8-1, one person skilled in the art can adopt other applicable starting materials (e.g., the starting materials with different choices of Y¹ to Y³ and the starting materials with different choices of L¹ and L²) and successfully synthesize other desired intermediates through a reaction mechanism similar to Scheme A1-1.

For example, other applicable starting materials may be, but are not limited to, the following reactants.

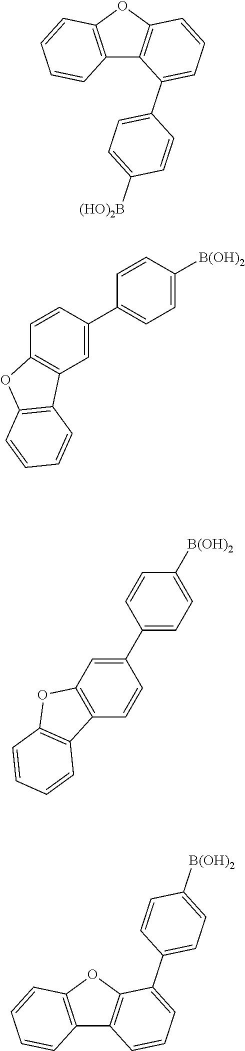 [SMILES: OB(O)c1ccc(-c2cccc3oc4ccccc4c23)cc1 ; OB(O)c1ccc(-c2ccc3oc4ccccc4c3c2)cc1 ; OB(O)c1ccc(-c2ccc3c(c2)oc2ccccc23)cc1 ; OB(O)c1ccc(-c2cccc3c2oc2ccccc23)cc1]

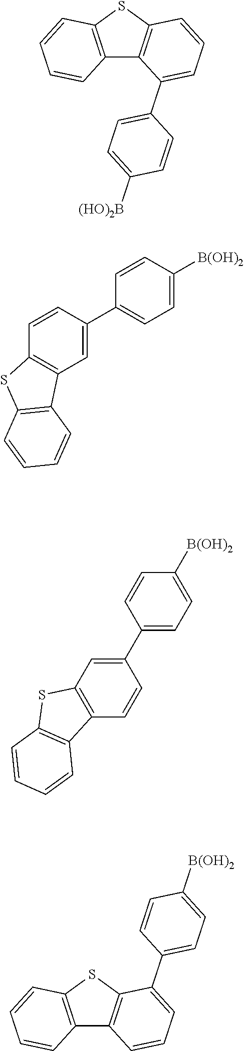 [SMILES: OB(O)c1ccc(-c2cccc3sc4ccccc4c23)cc1 ; OB(O)c1ccc(-c2ccc3sc4ccccc4c3c2)cc1 ; OB(O)c1ccc(-c2ccc3c(c2)sc2ccccc23)cc1 ; OB(O)c1ccc(-c2cccc3c2sc2ccccc23)cc1]

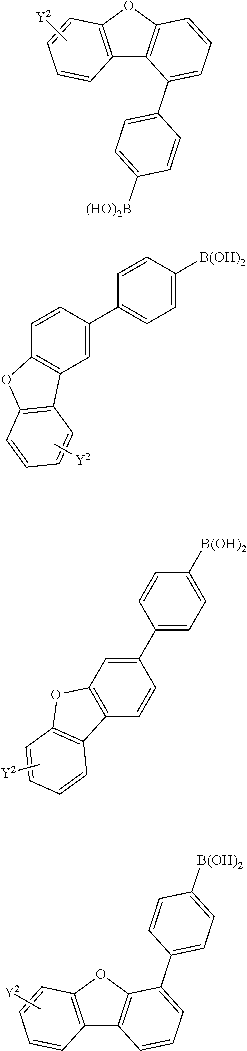

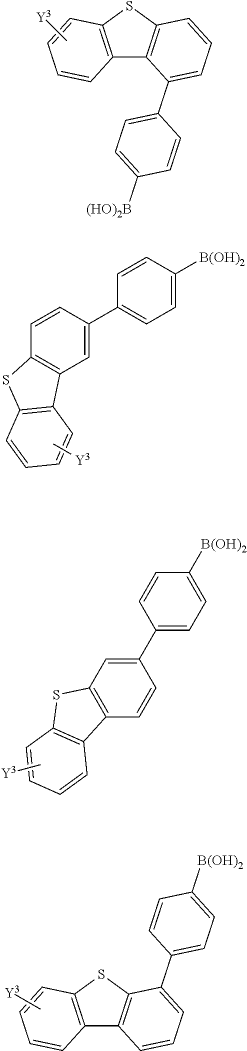

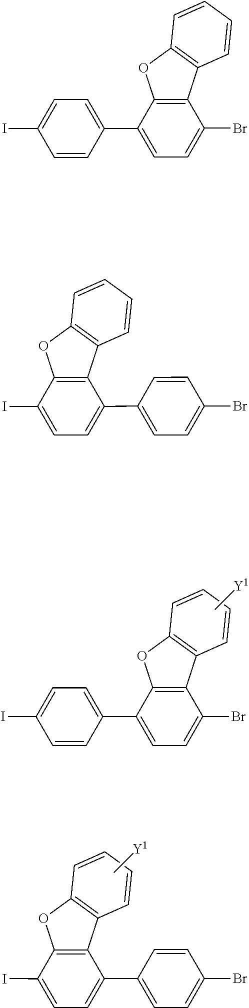

### Synthesis of Intermediate An

In step 2, the general synthesis pathway of Intermediate An was summarized in Scheme A2.

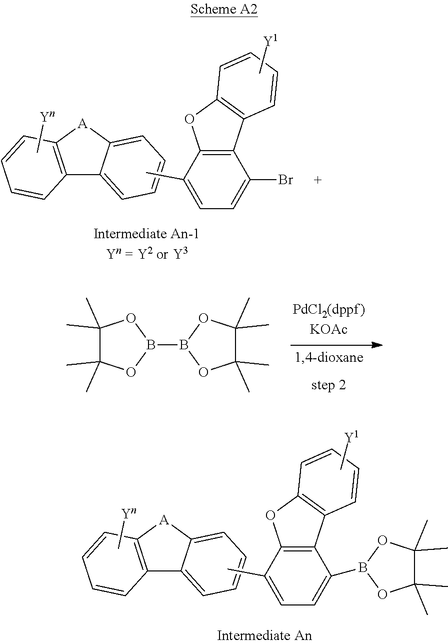

In Scheme A2, A is oxygen or sulfur; Y¹ to Y³ are each independently selected from the group consisting of: a hydrogen atom, a deuterium atom, an alkyl group having 1 to 12 carbon atoms, and an aryl group having 6 to 30 ring carbon atoms, and Y¹ to Y³ are the same or different.

### Synthesis of Intermediate A1

Taking Intermediate A1 as an example of Intermediate An, the synthesis pathway of Intermediate A1 was summarized in Scheme A2-1.

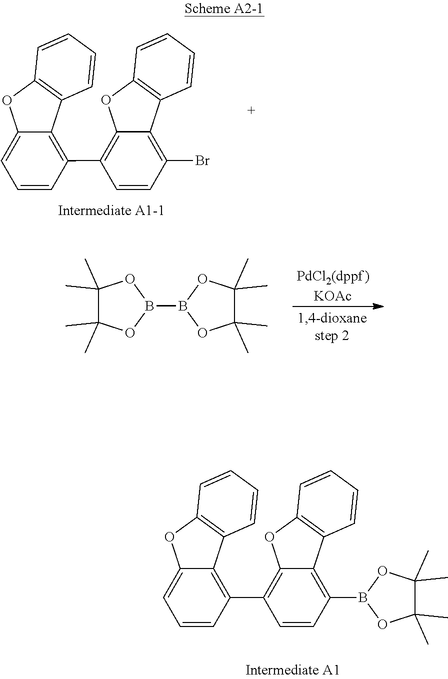

A mixture of Intermediate A1-1 (1.0 eq), bis(pinacolato)diboron (1.20 eq), 1,1′-bis(diphenylphosphino)-ferrocene dichloropalladium (II) [PdCl₂(dppf)] (0.025 eq), and potassium acetate (KOAc) (3.0 eq) in 1,4-dioxane (0.5 M) was degassed with nitrogen and then heated at about 90° C. for 16 hours. After cooling to room temperature, the precipitated crude product was separated by filtration to obtain a crude product. Then, the crude product was purified by column chromatography on silica gel with CH₂Cl₂/hexane (1:1 v/v) as eluent, and the eluent was concentrated under reduced pressure and then recrystallized with hexane to obtain a white solid product in 89.0% yield.

The white solid product was identified as Intermediate A1 by a FD-MS analysis. FD-MS analysis: C₃H₂₅BO₄; theoretical value: 460.18; observed value: 460.18.

### Syntheses of Intermediates A2 to A8

Intermediates A2 to A8, which also can be used for preparing a novel compound, were respectively synthesized in a similar manner as Intermediate A1 through step 2, except that the starting material Intermediate A1-1 was replaced by Intermediates A2-1 to A8-1, respectively. All intermediates were analyzed as described above, and the results were listed in Table 2.

TABLE 2
The chemical structures, yields, formulae, and mass analyzed by
FD-MS of Intermediates Al to A8.

| Intermediate | Chemical Structure of | Yield | Formula/ |
|---|---|---|---|
| An No. | Intermediate An | (%) | Mass (M⁺) |
| A1 | 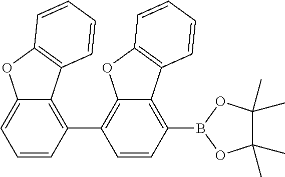 [SMILES: CC1(C)OB(c2ccc(-c3cccc4oc5ccccc5c34)c3oc4ccccc4c23)OC1(C)C] | 89.0 | C₃₀H₂₅BO₄/ 460.18 |
| A2 | 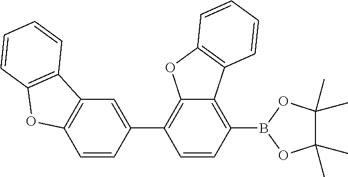 [SMILES: CC1(C)OB(c2ccc(-c3ccc4oc5ccccc5c4c3)c3oc4ccccc4c23)OC1(C)C] | 91.4 | C₃₀H₂₅BO₄/ 460.18 |
| A3 | 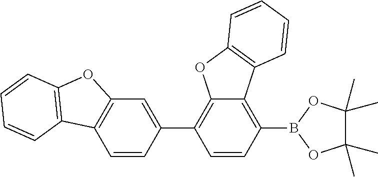 [SMILES: CC1(C)OB(c2ccc(-c3ccc4c(c3)oc3ccccc34)c3oc4ccccc4c23)OC1(C)C] | 90.7 | C₃₀H₂₅BO₄/ 460.18 |
| A4 | 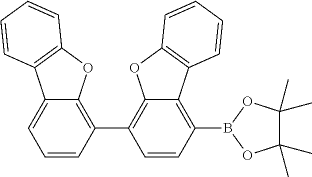 [SMILES: CC1(C)OB(c2ccc(-c3cccc4c3oc3ccccc34)c3oc4ccccc4c23)OC1(C)C] | 93.2 | C₃₀H₂₅BO₄/ 460.18 |
| A5 | 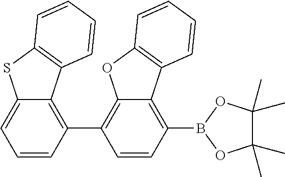 [SMILES: CC1(C)OB(c2ccc(-c3cccc4sc5ccccc5c34)c3oc4ccccc4c23)OC1(C)C] | 89.3 | C₃₀H₂₅BO₃S/ 476.16 |
| A6 | 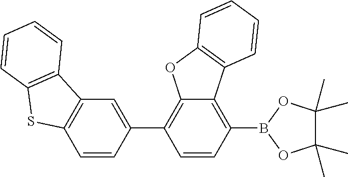 [SMILES: CC1(C)OB(c2ccc(-c3ccc4sc5ccccc5c4c3)c3oc4ccccc4c23)OC1(C)C] | 92.4 | C₃₀H₂₅BO₃S/ 476.16 |
| A7 | 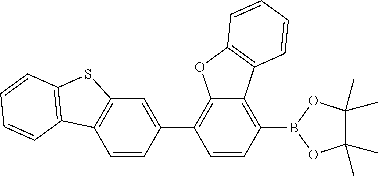 [SMILES: CC1(C)OB(c2ccc(-c3ccc4c(c3)sc3ccccc34)c3oc4ccccc4c23)OC1(C)C] | 91.7 | C₃₀H₂₅BO₃S/ 476.16 |
| A8 | 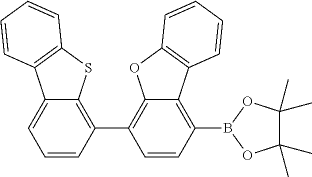 [SMILES: CC1(C)OB(c2ccc(-c3cccc4c3sc3ccccc34)c3oc4ccccc4c23)OC1(C)C] | 92.5 | C₃₀H₂₅BO₃S/ 476.16 |

Intermediate An used for preparing a novel compound can also be synthesized by the following steps.

### Another Synthesis of Intermediate an-1

In step 1′, the general synthesis pathway of Intermediate An-1 was summarized in Scheme A3, which can be used to prepare Intermediates A9 to A16.

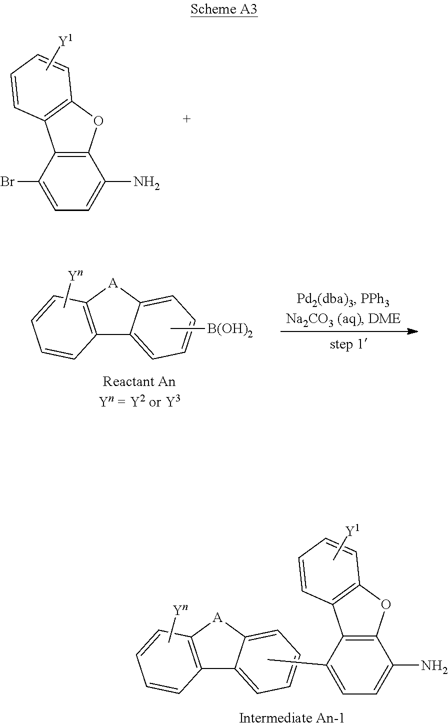

In Scheme A3, A is oxygen or sulfur; Y¹ to Y³ are each independently selected from the group consisting of: a hydrogen atom, a deuterium atom, an alkyl group having 1 to 12 carbon atoms, and an aryl group having 6 to 30 ring carbon atoms, and Y¹ to Y³ are the same or different.

### Synthesis of Intermediate A9-1

Taking Intermediate A9-1 as an example of Intermediate An-1, the synthesis pathway of Intermediate A9-1 was summarized in Scheme A3-1.

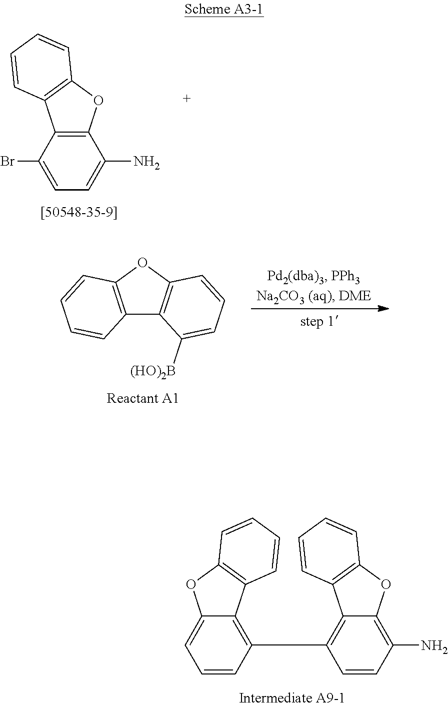 [SMILES: Nc1ccc(Br)c2c1oc1ccccc12 ; OB(O)c1cccc2oc3ccccc3c12 ; Nc1ccc(-c2cccc3oc4ccccc4c23)c2c1oc1ccccc12]

A mixture of 1-bromo-4-aminodibenzofuran (1.0 eq), 1-dibenzofuranboronic acid (1.0 eq), tris(dibenzylideneacetone)dipalladium[Pd₂(dba)₃] (0.005 eq), and triphenylphosphine (PPh₃) (0.02 eq) was placed in a mixed solution of methoxymethane (DME) (0.5 M) and Na₂CO₃ aqueous solution (2.0 M). Afterward, the following synthetic procedures were in a same manner as stated in Scheme A1-1. A white solid product was obtained in 69.2% yield.

The product was identified as Intermediate A9-1 by a FD-MS analysis. FD-MS analysis: C₂₄H₁₅NO₂; theoretical value: 349.11; observed value: 349.11.

### Syntheses of Intermediates A10-1 to A16-1

Intermediates A10-1 to A16-1, which also can be used for preparing a novel compound, were respectively synthesized in a similar manner as Intermediate A9-1 through step 1′, except that the starting material Reactant A1 was replaced by Reactants A2 to A8, respectively. All intermediates were analyzed as described above, and the results were listed in Table 3.

TABLE 3
The chemical structures of Reactant An used for preparing
Intermediates A9-1 to A16-1, and the chemical structures, yields, formulae, and
mass analyzed by FD-MS of Intermediates A9-1 to A16-1.

| Chemical Structure | Chemical Structure of | Yield | Formula/ |
|---|---|---|---|
| of Reactant An | Intermediate An-1 | (%) | Mass (M⁺) |
| 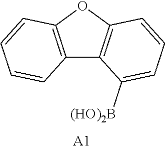 | 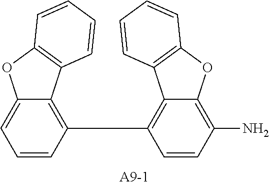 [SMILES: Nc1ccc(-c2cccc3oc4ccccc4c23)c2c1oc1ccccc12] | 69.2 | C₂₄H₁₅NO₂/ 349.11 |
| 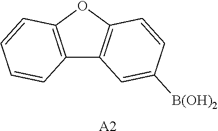 | 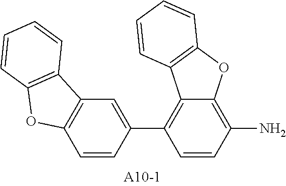 [SMILES: Nc1ccc(-c2ccc3oc4ccccc4c3c2)c2c1oc1ccccc12] | 73.2 | C₂₄H₁₅NO₂/ 349.11 |
| 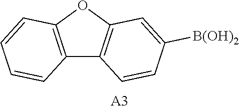 | 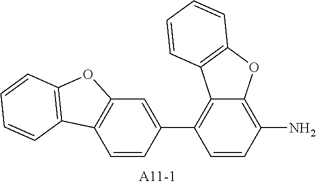 [SMILES: Nc1ccc(-c2ccc3c(c2)oc2ccccc23)c2c1oc1ccccc12] | 72.1 | C₂₄H₁₅NO₂/ 349.11 |
| 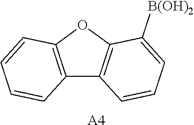 | 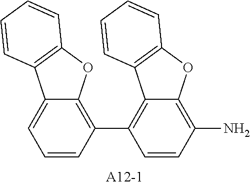 [SMILES: Nc1ccc(-c2cccc3c2oc2ccccc23)c2c1oc1ccccc12] | 73.0 | C₂₄H₁₅NO₂/ 349.11 |
| 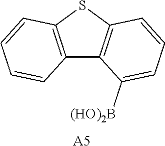 | 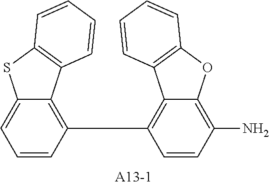 [SMILES: Nc1ccc(-c2cccc3sc4ccccc4c23)c2c1oc1ccccc12] | 68.5 | C₂₄H₁₅NOS/ 365.09 |
| 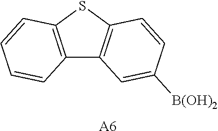 | 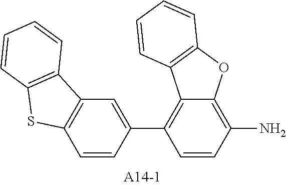 [SMILES: Nc1ccc(-c2ccc3sc4ccccc4c3c2)c2c1oc1ccccc12] | 72.5 | C₂₄H₁₅NOS/ 365.09 |
| 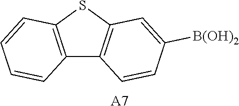 | 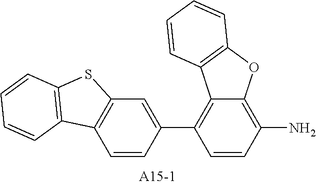 [SMILES: Nc1ccc(-c2ccc3c(c2)sc2ccccc23)c2c1oc1ccccc12] | 70.7 | C₂₄H₁₅NOS/ 365.09 |
| 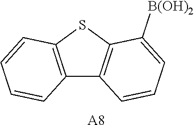 | 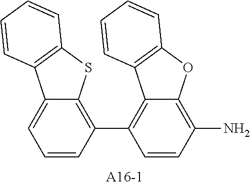 [SMILES: Nc1ccc(-c2cccc3c2sc2ccccc23)c2c1oc1ccccc12] | 71.8 | C₂₄H₁₅NOS/ 365.09 |

Modifications of Intermediates A9-1 to A16-1

In addition to Intermediates A9-1 to A16-1, one person skilled in the art can adopt other applicable starting materials (e.g., the starting materials with different choices of Y¹ to Y³ and the starting materials with different choices of L¹ and L²) and successfully synthesize other desired intermediates through a reaction mechanism similar to Scheme A3-1.

For example, other applicable starting materials may be, but are not limited to, the following reactants.

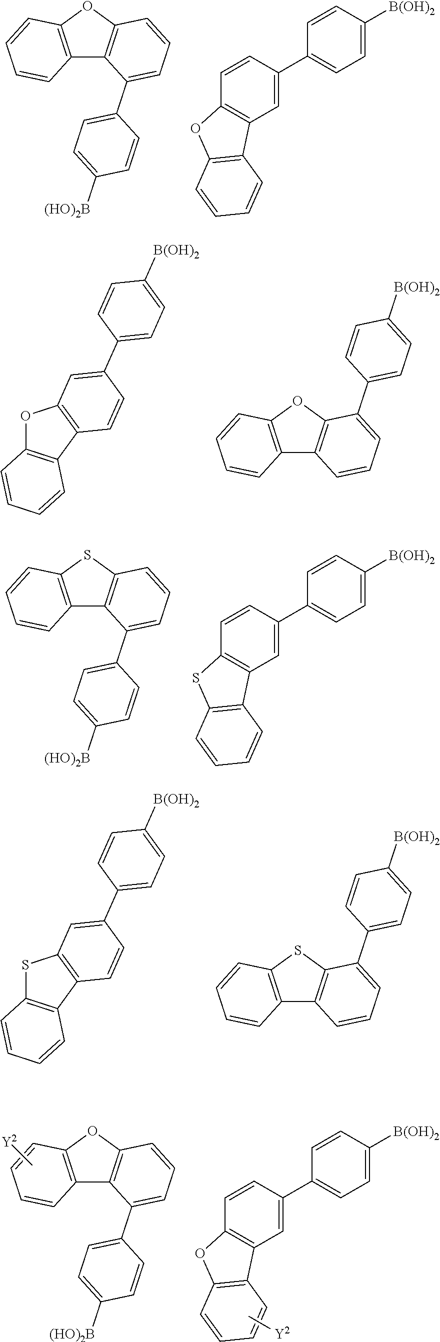

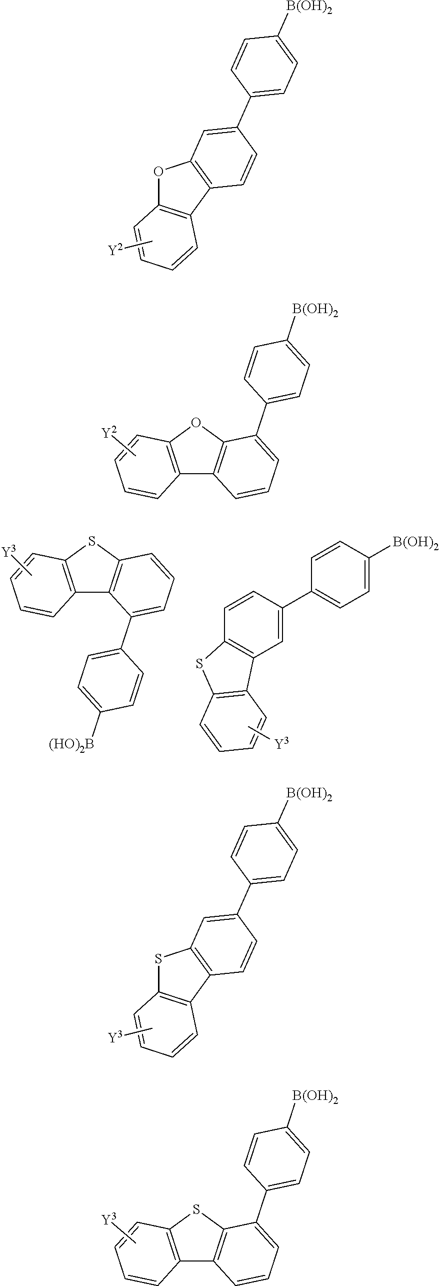

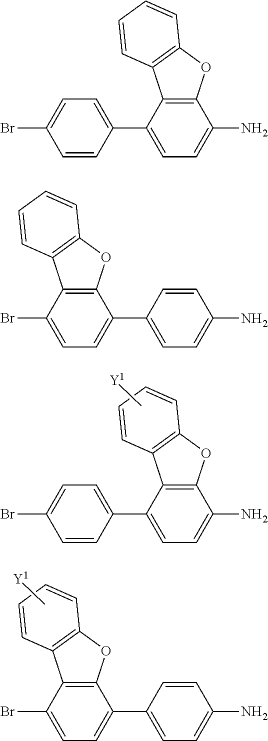

### Synthesis of Intermediate An

In step 2′-1 and step 2′-2, the general synthesis pathway of Intermediate An was summarized in Scheme A4.

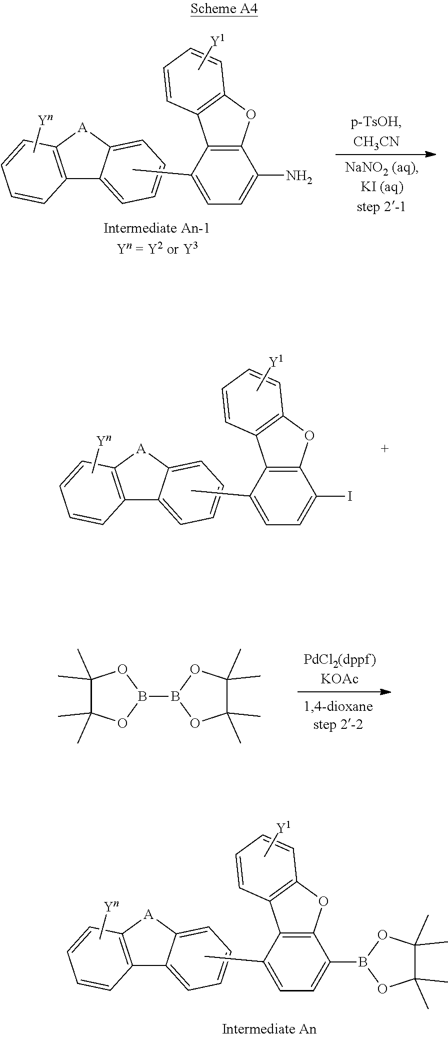

In Scheme A4, A is oxygen or sulfur; Y¹ to Y³ are each independently selected from the group consisting of: a hydrogen atom, a deuterium atom, an alkyl group having 1 to 12 carbon atoms, and an aryl group having 6 to 30 ring carbon atoms, and Y¹ to Y³ are the same or different.

### Synthesis of Intermediate A9

Taking Intermediate A9 as an example of Intermediate An, the synthesis pathway of Intermediate A9 was summarized in Scheme A4-1.

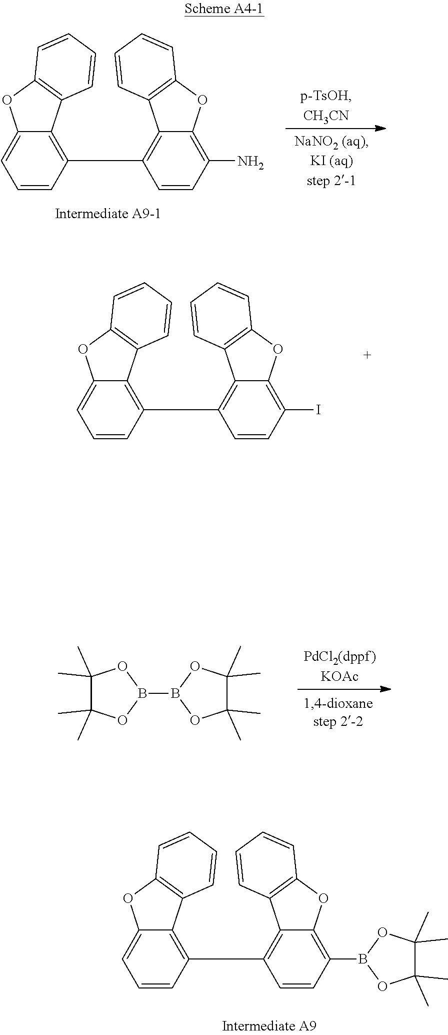

Intermediate A9-1 (1.0 eq) was added into a solution mixed with p-Toluenesulfonic acid⋅H₂O (p-TsOH.H₂O) (3.0 eq) and CH₃CN (0.5 M). Afterward, the mixed solution resulting with suspension of amine salt was cooled to below 10° C., and then an aqueous solution of NaNO₂ (2.0 eq) and KI (2.5 eq) was gradually added to the foresaid cooled solution, and the reaction mass was stirred for 1 hour, and then its temperature was raised to 20° C. and the reaction mass was stirred overnight. After that, the pH value of the solution was adjusted by saturated solution of NaHCO₃ until the pH value of the solution was between 9 and 10. The precipitate was separated by filtration or extracted with CH₂Cl₂, and then purified by flash chromatography with eluent (hexane to CH₂Cl₂ is 3 to 1) to obtain a crude solid product.

A mixture of the crude solid product (1.0 eq), bis(pinacolato)diboron (1.20 eq), 1,1′-bis(diphenylphosphino)-ferrocene dichloropalladium (II) [PdCl₂(dppf)] (0.025 eq), and potassium acetate (KOAc) (3.0 eq) in 1,4-dioxane (0.5 M) was degassed with nitrogen and then heated at about 90° C. for 16 hours. Afterward, the following synthetic procedures are in a same manner as stated in Scheme A2-1. A white solid product was obtained in 55.6% yield.

The white solid product was identified as Intermediate A9 by a FD-MS analysis. FD-MS analysis: C₃₀H₂₅BO₄; theoretical value: 460.18; observed value: 460.18.

### Syntheses of Intermediates A10 to A16

Intermediates A10 to A16, which also can be used for preparing a novel compound, were respectively synthesized in a similar manner as Intermediate A9 through step 2′-1 and step 2′-2, except that the starting material Intermediate A9-1 was replaced by Intermediates A10-1 to A16-1, respectively. All intermediates were analyzed as described above, and the results were listed in Table 4.

In Table 4, the yields of A9 to A16 were calculated by multiplying the yield of the step 2′-1 (65.6% to 71.4%) and the yield of the step 2′-2 (88.6% to 93.5%) in Scheme A4-1.

TABLE 4
The chemical structures, yields, formulae, and mass analyzed by
FD-MS of Intermediates A9 to A16.

| Intermediate | Chemical Structure of | Yield | Formula/ |
|---|---|---|---|
| An No. | Intermediate An | (%) | Mass (M⁺) |
| A9 | 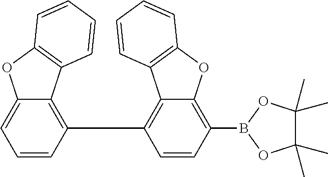 [SMILES: CC1(C)OB(c2ccc(-c3cccc4oc5ccccc5c34)c3c2oc2ccccc23)OC1(C)C] | 55.6 | C₃₀H₂₅BO₄/ 460.18 |
| A10 | 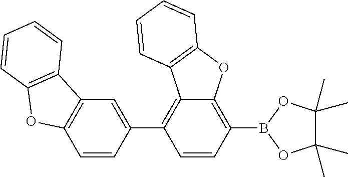 [SMILES: CC1(C)OB(c2ccc(-c3ccc4oc5ccccc5c4c3)c3c2oc2ccccc23)OC1(C)C] | 66.7 | C₃₀H₂₅BO₄/ 460.18 |
| A11 | 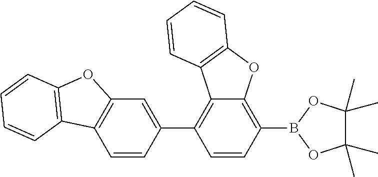 [SMILES: CC1(C)OB(c2ccc(-c3ccc4c(c3)oc3ccccc34)c3c2oc2ccccc23)OC1(C)C] | 59.8 | C₃₀H₂₅BO₄/ 460.18 |
| A12 | 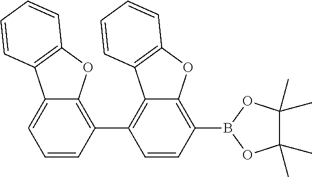 [SMILES: CC1(C)OB(c2ccc(-c3cccc4c3oc3ccccc34)c3c2oc2ccccc23)OC1(C)C] | 62.5 | C₃₀H₂₅BO₄/ 460.18 |
| A13 | 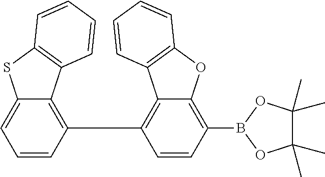 [SMILES: CC1(C)OB(c2ccc(-c3cccc4sc5ccccc5c34)c3c2oc2ccccc23)OC1(C)C] | 58.1 | C₃₀H₂₅BO₃S/ 476.16 |
| A14 | 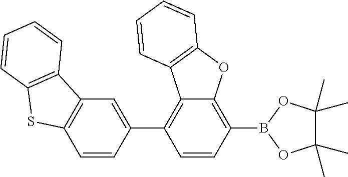 [SMILES: CC1(C)OB(c2ccc(-c3ccc4sc5ccccc5c4c3)c3c2oc2ccccc23)OC1(C)C] | 64.6 | C₃₀H₂₅BO₃S/ 476.16 |
| A15 | 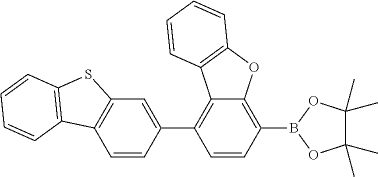 [SMILES: CC1(C)OB(c2ccc(-c3ccc4c(c3)sc3ccccc34)c3c2oc2ccccc23)OC1(C)C] | 62.1 | C₃₀H₂₅BO₃S/ 476.16 |
| A16 | 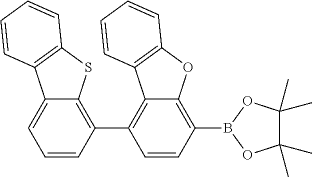 [SMILES: CC1(C)OB(c2ccc(-c3cccc4c3sc3ccccc34)c3c2oc2ccccc23)OC1(C)C] | 65.4 | C₃₀H₂₅BO₃S/ 476.16 |

Modifications of Intermediates A1 to A16

In addition to the foresaid synthesis pathway, modification of Intermediates A1 to A16 also can be implemented by the following summarized synthesis pathway.

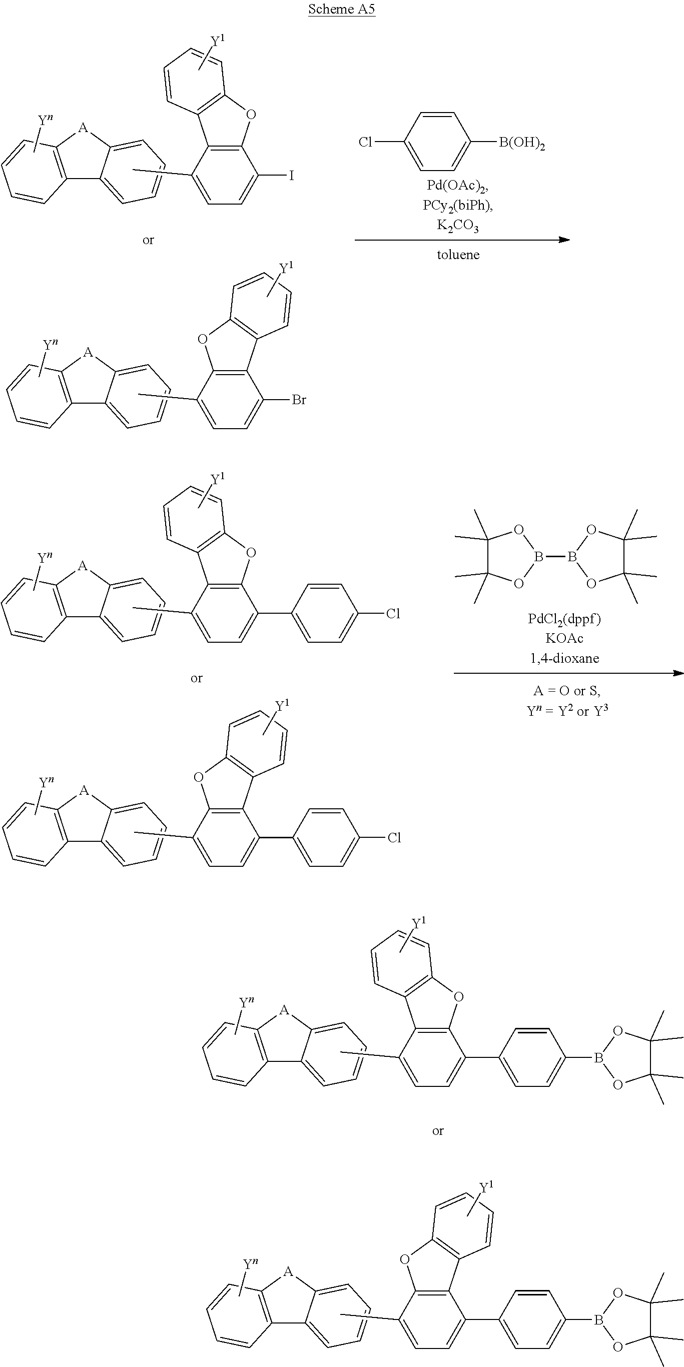

In Scheme A5, A is oxygen or sulfur; Y¹ to Y³ are each independently selected from the group consisting of: a hydrogen atom, a deuterium atom, an alkyl group having 1 to 12 carbon atoms, and an aryl group having 6 to 30 ring carbon atoms, and Y¹ to Y³ are the same or different.

For more detailed descriptions, an intermediate was prepared as follows.

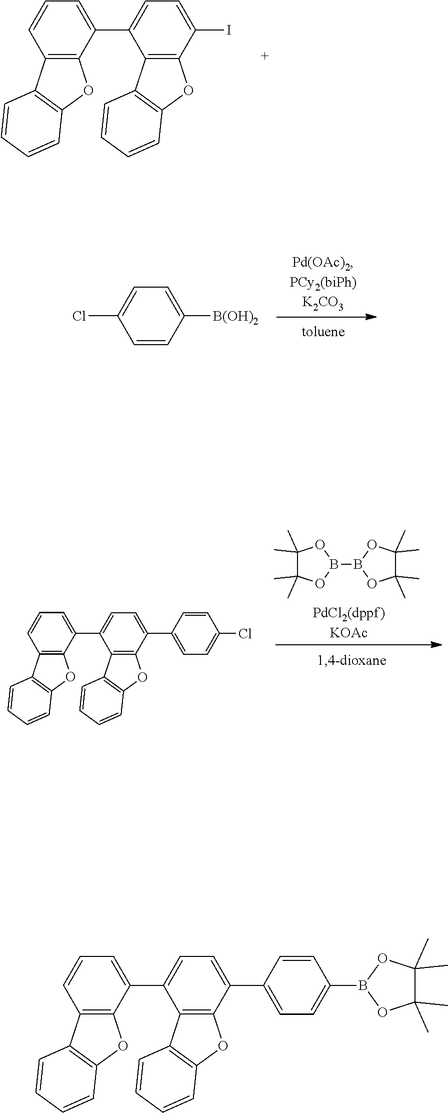

A mixture of (1-(dibenzofuran-4-yl)-4-iododibenzofuran) (50.0 g 1.0 eq), 4-chlorophenylboronic acid (1.05 eq, CAS No. 1679-18-1), Pd(OAc)₂ (0.01 eq), PCy₂(2-biPh) (0.04 eq), and K₂CO₃ (2.0 eq) was placed in a mixed solution of toluene (340 mL), ethanol (34 mL) and H₂O (72 mL). The reaction mixture was heated to about 80° C. under reflux and stirred for 16 hours under nitrogen atmosphere. After the completion of the reaction, the reaction mixture was cooled to room temperature, and the crude product was extracted and collected by the organic layer. The organic layer was dried over MgSO₄, separated by filtration and concentrated to dryness. A resulting residue was purified by silica gel column chromatography to obtain 43 g of white solid product in an yield of 89%.

The white solid product was identified by a FD-MS analysis. FD-MS analysis: C₃₀H₁₇ClO₂; theoretical value: 444.91; observed value: 444.91.

### Synthesis of Novel Compounds

Each of the foresaid Intermediates, e.g., Intermediates An could be reacted with various reactants to synthesize various claimed novel compounds. The general synthesis pathway of the claimed novel compound was summarized in Scheme I. In the following Scheme I, “Reactant Bn” may be any one of Reactants B1 to B9 and B9′ as listed in Table 5, and “Intermediate A” may be any one of the foresaid Intermediates An or the like. The compounds were each synthesized by the following steps.

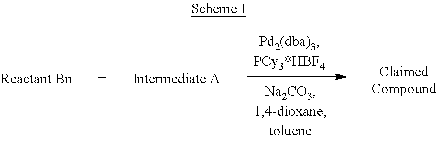

TABLE 5
The chemical structures and CAS No. of Reactants B1 to B9 and B9′.

| Reactant | | | | |
|---|---|---|---|---|
| No. | Reactant B1 | Reactant B2 | Reactant B3 | Reactant B4 |
| Chemical Structure | 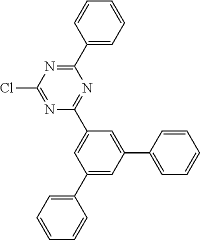 [SMILES: Clc1nc(-c2ccccc2)nc(-c2cc(-c3ccccc3)cc(-c3ccccc3)c2)n1] | 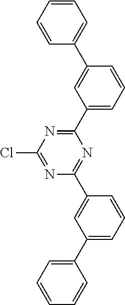 [SMILES: Clc1nc(-c2cccc(-c3ccccc3)c2)nc(-c2cccc(-c3ccccc3)c2)n1] | 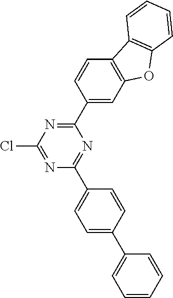 [SMILES: Clc1nc(-c2ccc(-c3ccccc3)cc2)nc(-c2ccc3c(c2)oc2ccccc23)n1] | 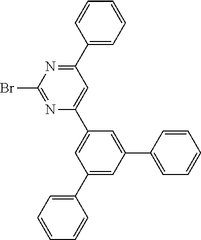 [SMILES: Brc1nc(-c2ccccc2)cc(-c2cc(-c3ccccc3)cc(-c3ccccc3)c2)n1] |
| CAS No. | 1616231-57-2 | 1205748-61-3 | 2170887-83-7 | 2021249-58-9 |

| Reactant | | | | |
|---|---|---|---|---|
| No. | Reactant B5 | Reactant B6 | Reactant B7 | Reactant B8 |
| Chemical Structure | 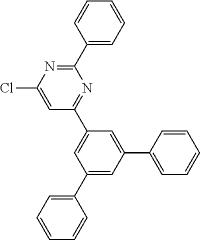 [SMILES: Clc1cc(-c2cc(-c3ccccc3)cc(-c3ccccc3)c2)nc(-c2ccccc2)n1] | 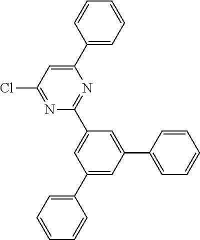 [SMILES: Clc1cc(-c2ccccc2)nc(-c2cc(-c3ccccc3)cc(-c3ccccc3)c2)n1] | 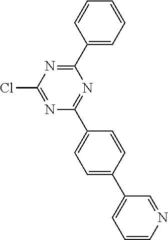 [SMILES: Clc1nc(-c2ccccc2)nc(-c2ccc(-c3cccnc3)cc2)n1] | 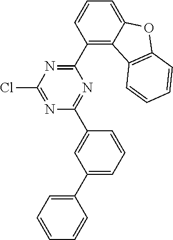 [SMILES: Clc1nc(-c2cccc(-c3ccccc3)c2)nc(-c2cccc3oc4ccccc4c23)n1] |
| CAS No. | 1852465-84-9 | 1934308-81-2 | 2074632-12-3 | 1883265-36-8 |

| Reactant | | |
|---|---|---|
| No. | Reactant B9 | Reactant B9′ |
| Chemical Structure | 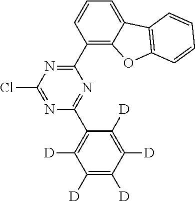 [SMILES: [2H]c1c([2H])c([2H])c(-c2nc(Cl)nc(-c3cccc4c3oc3ccccc34)n2)c([2H])c1[2H]] | 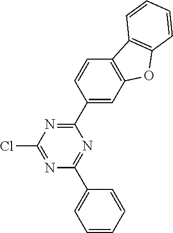 [SMILES: Clc1nc(-c2ccccc2)nc(-c2ccc3c(c2)oc2ccccc23)n1] |
| CAS No. | 2408705-74-6 | 2142681-84-1 |

Scheme I

In Scheme I, a mixture of Intermediate A (1.0 eq), Reactant Bn (1.0 eq), Pd₂(dba)₃ (0.01 eq), PCy₃*HBF₄ (0.02 eq), sodium carbonate solution (2.0 M) in 1,4-dioxane/toluene (2:1 v/v) as solvent was refluxed for about 12 to 16 hours. After the completion of the reaction, the reaction mixture was cooled to room temperature, and the precipitated crude product was then separated by filtration to obtain a crude product. After the filtration, the crude product was purified by recrystallization method using otho-dichlorobenzene to obtain a white solid product as the claimed novel compounds.

Another synthesis pathway of the claimed novel compound was summarized in Scheme II. In the following Scheme II, “Reactant Bn” may be any one of Reactants B10 to B11 as listed in Table 6, and “Intermediate A” may be any one of the foresaid Intermediates An or the like. The compounds were each synthesized by the following steps.

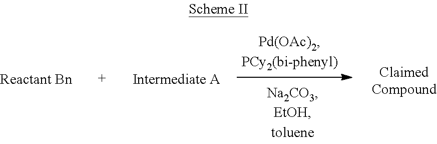

TABLE 6
The chemical structures and CAS No. of Reactants B10 to B11.

| Reactant No. | Reactant B10 | Reactant B11 |
|---|---|---|
| Chemical Structure | 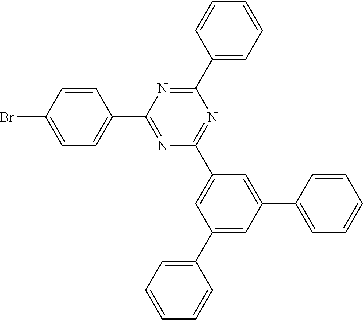 [SMILES: Brc1ccc(-c2nc(-c3ccccc3)nc(-c3cc(-c4ccccc4)cc(-c4ccccc4)c3)n2)cc1] | 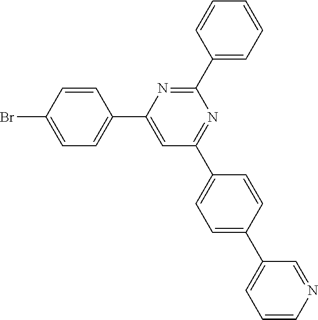 [SMILES: Brc1ccc(-c2cc(-c3ccc(-c4cccnc4)cc3)nc(-c3ccccc3)n2)cc1] |
| CAS No. | 2305965-85-7 | 1776082-96-2 |

Scheme II

In Scheme II, a mixture of Intermediate A (1.0 eq), Reactant Bn (1.0 eq), Pd(OAc)₂ (0.01 eq), PCy₂(2-bi-phenyl) (0.02 eq), sodiumcarbonate solution (2.0 M) in toluene/EtOH (1:0.1 v/v) as solvent was refluxed for about 8 to 12 hours. After the completion of the reaction, the reaction mixture was cooled to room temperature, and the precipitated crude product was then separated by filtration to obtain a crude product. After the filtration, the crude product was purified by recrystallization method using otho-dichlorobenzene to obtain a white solid product as the claimed novel compounds.

Intermediate A and Reactant Bn adopted to synthesize the claimed novel compounds were listed in Table 7.

Compounds 1 to 20 were identified by ¹H-NMR and FD-MS, and the chemical structure, yield, formula, mass of each of Compounds 1 to 20 were also listed in Table 7. Also, the ¹H-NMR result of each of Compounds 1 to 5 and 7 to 20 were listed in Table 8.

TABLE 7
Reactants and Intermediates adopted to prepare Compounds 1
to 20 and their chemical structures, yields, formulae, and FD-MS data.

| | | Claimed Compound | | |
|---|---|---|---|---|
| Reactant | Intermediate | Chemical Structure of | Yield | Formula/ |
| Bn No. | An No. | Claimed Compound | (%) | Mass (M⁺) |
| B1 | A4 | 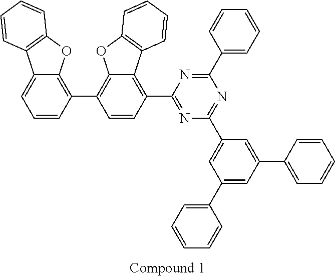 [SMILES: c1ccc(-c2cc(-c3ccccc3)cc(-c3nc(-c4ccccc4)nc(-c4ccc(-c5cccc6c5oc5ccccc56)c5oc6ccccc6c45)n3)c2)cc1] | 86.5 | C₅₁H₃₁N₃O₂/ 717.81 |
| B1 | A3 | 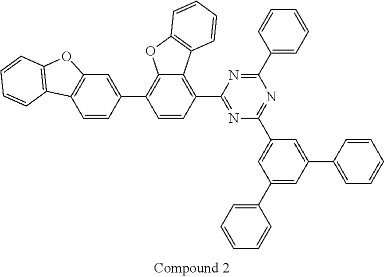 [SMILES: c1ccc(-c2cc(-c3ccccc3)cc(-c3nc(-c4ccccc4)nc(-c4ccc(-c5ccc6c(c5)oc5ccccc56)c5oc6ccccc6c45)n3)c2)cc1] | 84.7 | C₅₁H₃₁N₃O₂/ 717.81 |
| B1 | A2 | 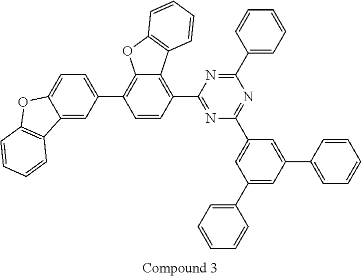 [SMILES: c1ccc(-c2cc(-c3ccccc3)cc(-c3nc(-c4ccccc4)nc(-c4ccc(-c5ccc6oc7ccccc7c6c5)c5oc6ccccc6c45)n3)c2)cc1] | 85.8 | C₅₁H₃₁N₃O₂/ 717.81 |
| B1 | A1 | 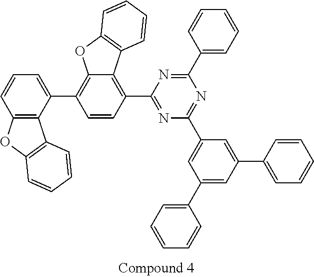 [SMILES: c1ccc(-c2cc(-c3ccccc3)cc(-c3nc(-c4ccccc4)nc(-c4ccc(-c5cccc6oc7ccccc7c56)c5oc6ccccc6c45)n3)c2)cc1] | 81.5 | C₅₁H₃₁N₃O₂/ 717.81 |
| B2 | A4 | 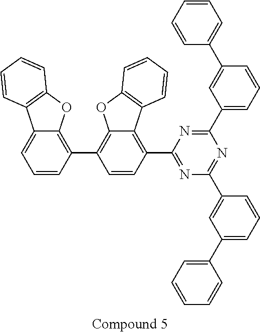 [SMILES: c1ccc(-c2cccc(-c3nc(-c4cccc(-c5ccccc5)c4)nc(-c4ccc(-c5cccc6c5oc5ccccc56)c5oc6ccccc6c45)n3)c2)cc1] | 87.3 | C₅₁H₃₁N₃O₂/ 717.81 |
| B3 | A4 | 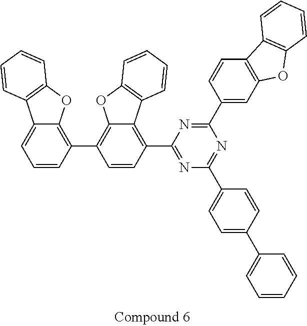 [SMILES: c1ccc(-c2ccc(-c3nc(-c4ccc5c(c4)oc4ccccc45)nc(-c4ccc(-c5cccc6c5oc5ccccc56)c5oc6ccccc6c45)n3)cc2)cc1] | 78.4 | C₅₁H₂₉N₃O₃/ 731.79 |
| B1 | A10 | 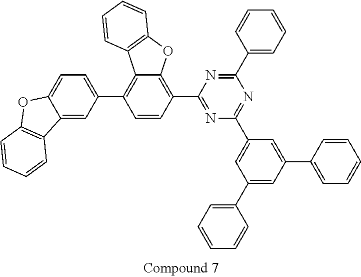 [SMILES: c1ccc(-c2cc(-c3ccccc3)cc(-c3nc(-c4ccccc4)nc(-c4ccc(-c5ccc6oc7ccccc7c6c5)c5c4oc4ccccc45)n3)c2)cc1] | 84.5 | C₅₁H₃₁N₃O₂/ 717.81 |
| B10 | A11 | 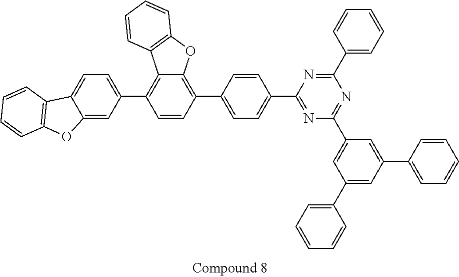 [SMILES: c1ccc(-c2cc(-c3ccccc3)cc(-c3nc(-c4ccccc4)nc(-c4ccc(-c5ccc(-c6ccc7c(c6)oc6ccccc67)c6c5oc5ccccc56)cc4)n3)c2)cc1] | 89.6 | C₅₇H₃₅N₃O₂/ 793.91 |
| B11 | A8 | 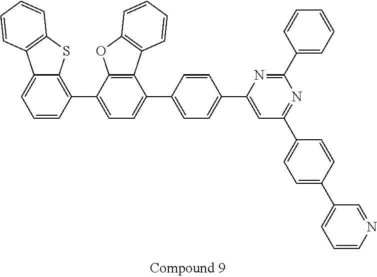 [SMILES: c1ccc(-c2nc(-c3ccc(-c4cccnc4)cc3)cc(-c3ccc(-c4ccc(-c5cccc6c5sc5ccccc56)c5oc6ccccc6c45)cc3)n2)cc1] | 87.3 | C₅₁H₃₁N₃OS/ 733.88 |
| B4 | A4 | 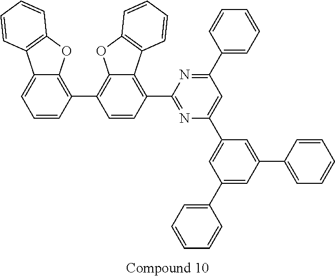 [SMILES: c1ccc(-c2cc(-c3ccccc3)cc(-c3cc(-c4ccccc4)nc(-c4ccc(-c5cccc6c5oc5ccccc56)c5oc6ccccc6c45)n3)c2)cc1] | 84.6 | C₅₂H₃₂N₂O₂/ 716.82 |
| B5 | A4 | 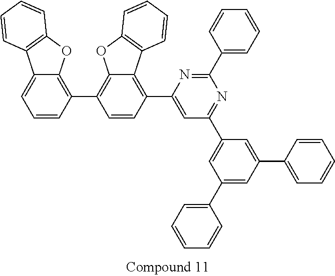 [SMILES: c1ccc(-c2cc(-c3ccccc3)cc(-c3cc(-c4ccc(-c5cccc6c5oc5ccccc56)c5oc6ccccc6c45)nc(-c4ccccc4)n3)c2)cc1] | 83.5 | C₅₂H₃₂N₂O₂/ 716.82 |
| B6 | A4 | 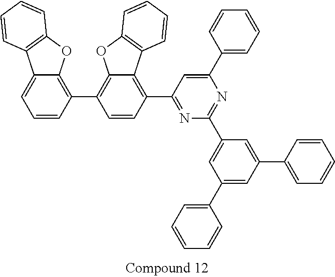 [SMILES: c1ccc(-c2cc(-c3ccccc3)cc(-c3nc(-c4ccccc4)cc(-c4ccc(-c5cccc6c5oc5ccccc56)c5oc6ccccc6c45)n3)c2)cc1] | 84.3 | C₅₂H₃₂N₂O₂/ 716.82 |
| B9′ | A12 | 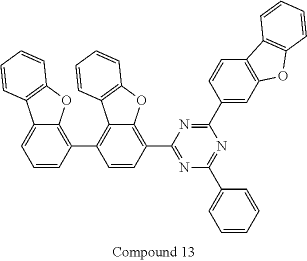 [SMILES: c1ccc(-c2nc(-c3ccc4c(c3)oc3ccccc34)nc(-c3ccc(-c4cccc5c4oc4ccccc45)c4c3oc3ccccc34)n2)cc1] | 79.8 | C₄₅H₂₅N₃O₃/ 655.70 |
| B5 | A12 | 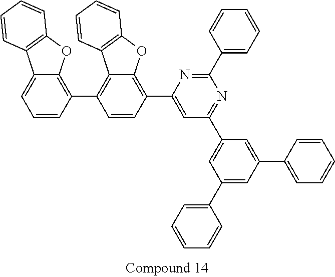 [SMILES: c1ccc(-c2cc(-c3ccccc3)cc(-c3cc(-c4ccc(-c5cccc6c5oc5ccccc56)c5c4oc4ccccc45)nc(-c4ccccc4)n3)c2)cc1] | 81.8 | C₅₂H₃₂N₂O₂/ 716.82 |
| B5 | A10 | 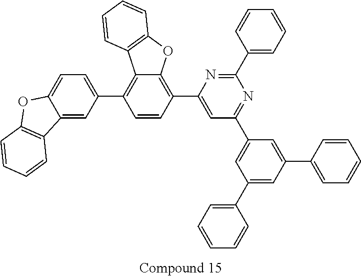 [SMILES: c1ccc(-c2cc(-c3ccccc3)cc(-c3cc(-c4ccc(-c5ccc6oc7ccccc7c6c5)c5c4oc4ccccc45)nc(-c4ccccc4)n3)c2)cc1] | 67.0 | C₅₂H₃₂N₂O₂/ 716.82 |
| B11 | A4 | 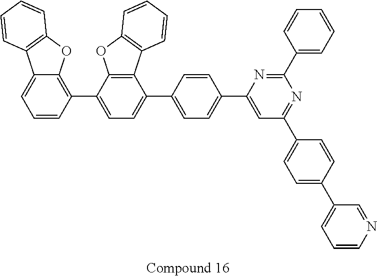 [SMILES: c1ccc(-c2nc(-c3ccc(-c4cccnc4)cc3)cc(-c3ccc(-c4ccc(-c5cccc6c5oc5ccccc56)c5oc6ccccc6c45)cc3)n2)cc1] | 88.6 | C₅₁H₃₁N₃O₂/ 717.81 |
| B1 | A8 | 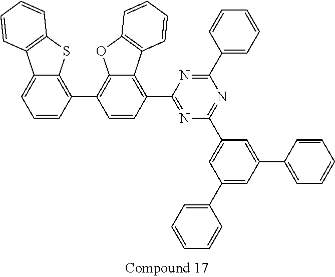 [SMILES: c1ccc(-c2cc(-c3ccccc3)cc(-c3nc(-c4ccccc4)nc(-c4ccc(-c5cccc6c5sc5ccccc56)c5oc6ccccc6c45)n3)c2)cc1] | 88.8 | C₅₁H₃₁N₃OS/ 733.88 |
| B5 | A6 | 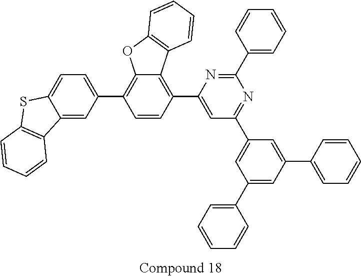 [SMILES: c1ccc(-c2cc(-c3ccccc3)cc(-c3cc(-c4ccc(-c5ccc6sc7ccccc7c6c5)c5oc6ccccc6c45)nc(-c4ccccc4)n3)c2)cc1] | 69.9 | C₅₂H₃₂N₂OS/ 732.89 |
| B9 | A16 | 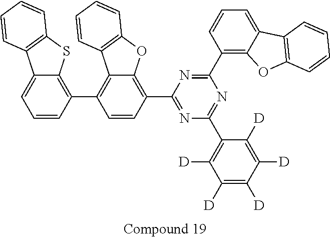 [SMILES: [2H]c1c([2H])c([2H])c(-c2nc(-c3cccc4c3oc3ccccc34)nc(-c3ccc(-c4cccc5c4sc4ccccc45)c4c3oc3ccccc34)n2)c([2H])c1[2H]] | 56.9 | C₄₅H₂₀D₅N₃O₂/ 676.79 |
| B1 | A14 | 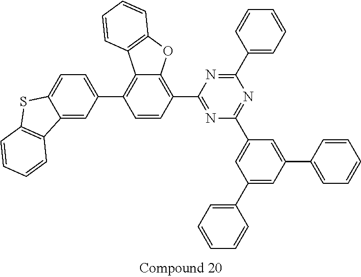 [SMILES: c1ccc(-c2cc(-c3ccccc3)cc(-c3nc(-c4ccccc4)nc(-c4ccc(-c5ccc6sc7ccccc7c6c5)c5c4oc4ccccc45)n3)c2)cc1] | 71.6 | C₅₁H₃₁N₃OS/ 733.88 |

TABLE 8
¹H-NMR results of Compounds 1 to 5 and 7 to 20.

| Claimed Compound | ¹H-NMR |
|---|---|
| 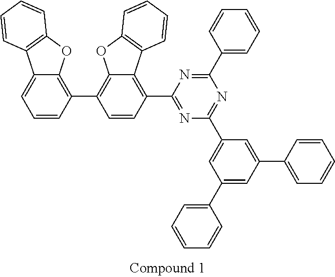 [SMILES: c1ccc(-c2cc(-c3ccccc3)cc(-c3nc(-c4ccccc4)nc(-c4ccc(-c5cccc6c5oc5ccccc56)c5oc6ccccc6c45)n3)c2)cc1] | ¹H NMR (500 MHz, CDCl₃): δ 9.04(dd, 2H), 8.85(dd, 2H), 8.73(d, 1H), 8.58(d, 1H), 8.15~8.00(m, 5H), 7.78(dd, 4H), 7.65~7.38(m, 16H) ppm. |
| 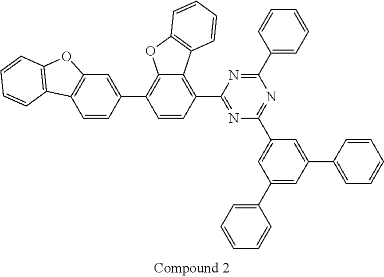 [SMILES: c1ccc(-c2cc(-c3ccccc3)cc(-c3nc(-c4ccccc4)nc(-c4ccc(-c5ccc6c(c5)oc5ccccc56)c5oc6ccccc6c45)n3)c2)cc1] | ¹H NMR (500 MHz, CDCl₃): δ 9.00(s, 2H), 8.80(d, 2H), 8.70(d, 1H), 8.48(d, 1H), 8.22(s, 1H), 8.10(d, 1H), 8.06(s,1H), 8.00(d, 1H), 7.92(d, 1H), 7.82(d, 1H), 7.75(d, 4H), 7.57-7.68(m, 5H), 7.48-7.51(m, 6H), 7.38-7.43(m, 3H), 7.23(m, 1H) ppm. |
| 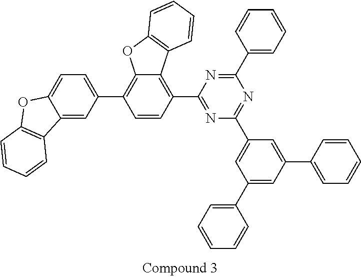 [SMILES: c1ccc(-c2cc(-c3ccccc3)cc(-c3nc(-c4ccccc4)nc(-c4ccc(-c5ccc6oc7ccccc7c6c5)c5oc6ccccc6c45)n3)c2)cc1] | ¹H NMR (500 MHz, CDCl₃): δ 9.02(dd, 2H), 8.83(dd, 2H), 8.73(d, 1H), 8.53(d, 2H), 8.12~8.03(m, 3H), 7.86-7.55(m, 12H), 7.53~7.36(m, 9H) ppm. |
| 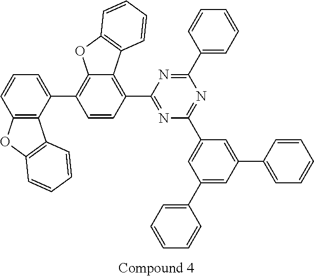 [SMILES: c1ccc(-c2cc(-c3ccccc3)cc(-c3nc(-c4ccccc4)nc(-c4ccc(-c5cccc6oc7ccccc7c56)c5oc6ccccc6c45)n3)c2)cc1] | ¹H NMR (500 MHz, CDCl₃): δ 8.97(dd, 4H), 8.79(dd, 2H), 8.04(d, 2H), 7.79(dd, 8H), 7.62~7.41(m, 15H) ppm. |
| 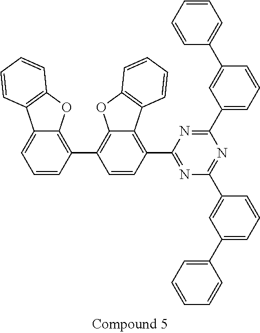 [SMILES: c1ccc(-c2cccc(-c3nc(-c4cccc(-c5ccccc5)c4)nc(-c4ccc(-c5cccc6c5oc5ccccc56)c5oc6ccccc6c45)n3)c2)cc1] | ¹H NMR (500 MHz, CDCl₃): δ 9.08(dd, 2H), 8.81(dd, 2H), 8.75(d, 1H), 8.58(d, 1H), 8.13(d, 1H), 8.07~8.00(m, 3H), 7.88(dd, 2H), 7.74(dd, 4H), 7.66(dd, 2H), 7.59~7.46(m, 9H), 7.40(m, 3H), 7.24(dd, 1H) ppm. |
| 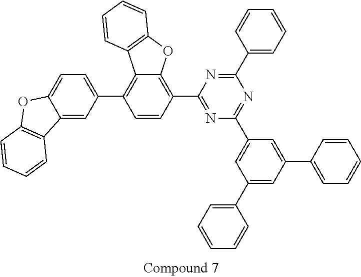 [SMILES: c1ccc(-c2cc(-c3ccccc3)cc(-c3nc(-c4ccccc4)nc(-c4ccc(-c5ccc6oc7ccccc7c6c5)c5c4oc4ccccc45)n3)c2)cc1] | ¹H NMR (500 MHz, CDCl₃): δ 9.13(dd, 2H), 8.87(dd, 2H), 8.23(d, 1H), 8.06(d, 1H), 7.97(dd, 1H), 7.85(dd, 4H), 7.80~7.35(m, 19H), 7.11(dd, 1H) ppm. |
| 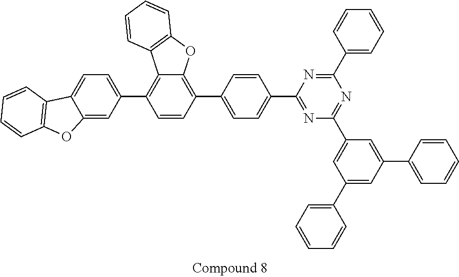 [SMILES: c1ccc(-c2cc(-c3ccccc3)cc(-c3nc(-c4ccccc4)nc(-c4ccc(-c5ccc(-c6ccc7c(c6)oc6ccccc67)c6c5oc5ccccc56)cc4)n3)c2)cc1] | ¹H NMR (500 MHz, CDCl₃): δ 9.01(d, 2H), 8.98(d, 2H), 8.83(dd, 2H), 8.20(d, 2H), 8.12(d, 1H), 8.07~8.02(m, 2H), 7.87~7.39(m, 23H), 7.12(dd, 1H) ppm. |
| 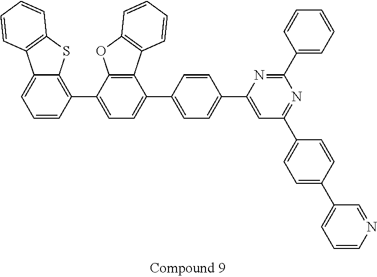 [SMILES: c1ccc(-c2nc(-c3ccc(-c4cccnc4)cc3)cc(-c3ccc(-c4ccc(-c5cccc6c5sc5ccccc56)c5oc6ccccc6c45)cc3)n2)cc1] | ¹H NMR (500 MHz, CDCl₃): δ 8.95(d, 1H), 8.80(dd, 2H), 8.63(d, 1H), 8.51(d, 2H), 8.44(d, 2H), 8.26(dd, 2H), 8.14(s, 1H), 7.95~7.89(m, 4H), 7.84~7.76(m, 4H), 7.72(d, 1H), 7.67(t, 1H), 7.60~7.52(m, 4H), 7.48~7.39(m, 5H), 7.17(t, 1H) ppm. |
| 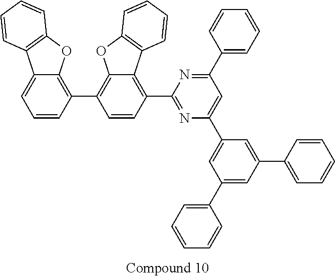 [SMILES: c1ccc(-c2cc(-c3ccccc3)cc(-c3cc(-c4ccccc4)nc(-c4ccc(-c5cccc6c5oc5ccccc56)c5oc6ccccc6c45)n3)c2)cc1] | ¹H NMR (500 MHz, CDCl₃): δ 8.58-8.53(m, 4H), 8.44(d, 1H), 8.39~8.36(m, 2H), 8.30(s, 1H), 8.11~8.00(m, 6H), 7.75~7.73(m, 5H), 7.60~7.36(m, 12H), 7.16(dd, 1H) ppm. |
| 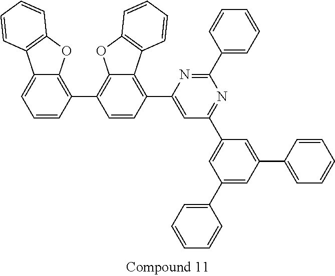 [SMILES: c1ccc(-c2cc(-c3ccccc3)cc(-c3cc(-c4ccc(-c5cccc6c5oc5ccccc56)c5oc6ccccc6c45)nc(-c4ccccc4)n3)c2)cc1] | ¹H NMR (500 MHz, CDCl₃): δ 8.79~8.77(m, 2H), 8.52(d, 2H), 8.22(s, 1H), 8.17~7.99(m, 6H), 7.87(d, 1H), 7.78(dd, 4H), 7.60~7.38(m, 15H), 7.20(dd, 1H) ppm. |
| 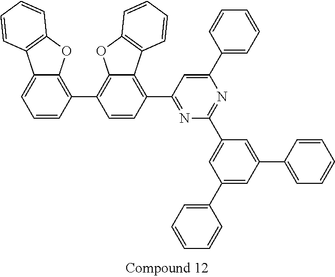 [SMILES: c1ccc(-c2cc(-c3ccccc3)cc(-c3nc(-c4ccccc4)cc(-c4ccc(-c5cccc6c5oc5ccccc56)c5oc6ccccc6c45)n3)c2)cc1] | ¹H NMR (500 MHz, CDCl₃): δ 8.99(d, 2H), 8.37(dd, 2H), 8.22(d, 1H), 8.16(s, 1H), 8.10~7.99(m, 5H), 7.87(d, 1H), 7.76(dd, 4H), 7.61~7.56(m, 6H), 7.49~7.44(m, 6H), 7.40~7.38(m, 3H), 7.22(dd, 1H) ppm. |
| 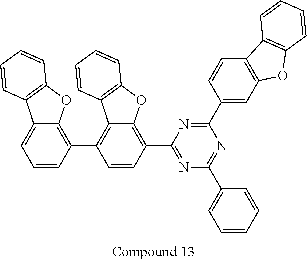 [SMILES: c1ccc(-c2nc(-c3ccc4c(c3)oc3ccccc34)nc(-c3ccc(-c4cccc5c4oc4ccccc45)c4c3oc3ccccc34)n2)cc1] | ¹H NMR (500 MHz, CDCl₃): δ 9.02(s, 1H), 8.83(t, 4H), 8.10(d, 1H), 8.06(t, 2H), 7.94(d, 1H), 7.76(d, 1H), 7.70(d, 1H), 7.65(d, 1H), 7.61(d, 4H), 7.50(d, 1H), 7.47(t, 1H), 7.43(m, 4H), 7.38(t, 1H), 7.04(d, 1H), 6.97(t, 1H) ppm. |
| 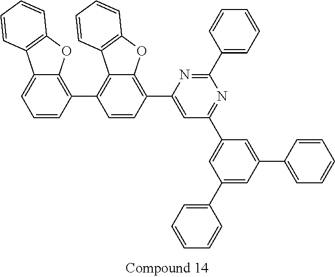 [SMILES: c1ccc(-c2cc(-c3ccccc3)cc(-c3cc(-c4ccc(-c5cccc6c5oc5ccccc56)c5c4oc4ccccc45)nc(-c4ccccc4)n3)c2)cc1] | ¹H NMR (500 MHz, CDCl₃): δ 9.0(s, 1H), 8.85-8.80(m, 3H), 8.57(d, 2H), 8.12(d, 1H), 8.04(d, 1H), 8.01(s, 1H), 7.82(d, 4H), 7.71(t, 2H), 7.63(d, 1H), 7.58~7.53(m, 9H), 7.46~7.39(m, 5H), 7.07~7.01(dd, 2H) ppm. |
| 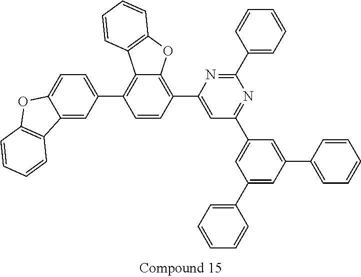 [SMILES: c1ccc(-c2cc(-c3ccccc3)cc(-c3cc(-c4ccc(-c5ccc6oc7ccccc7c6c5)c5c4oc4ccccc45)nc(-c4ccccc4)n3)c2)cc1] | ¹H NMR (500 MHz, CDCl₃): δ 8.97(s, 1H), 8.78(t, 3H), 8.56(d, 2H), 8.23(s, 1H), 7.9(d, 2H), 7.80(d, 4H), 7.75(s, 2H), 7.64(dd, 2H), 7.58~7.51(m, 9H), 7.49-7.42(q, 4H), 7.37(t, 1H), 7.12(t, 1H) ppm. |
| 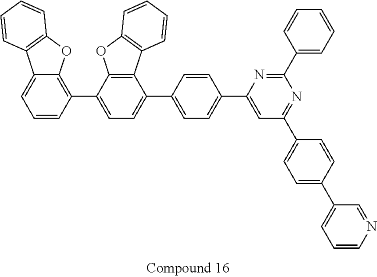 [SMILES: c1ccc(-c2nc(-c3ccc(-c4cccnc4)cc3)cc(-c3ccc(-c4ccc(-c5cccc6c5oc5ccccc56)c5oc6ccccc6c45)cc3)n2)cc1] | ¹H NMR (500 MHz, CDCl₃): δ 8.95(d, 1H), 8.80(dd, 2H), 8.64(dd, 1H), 8.50(d, 2H), 8.43(d, 2H), 8.16(s, 1H), 8.05-8.00(m, 4H), 7.94(dt, 1H), 7.90(d, 2H), 7.79(d, 2H), 7.69(d, 1H), 7.58~7.52(m, 5H), 7.48(t, 2H), 7.41~7.39(m, 4H), 7.17(t, 1H) ppm. |
| 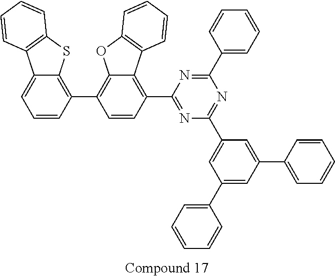 [SMILES: c1ccc(-c2cc(-c3ccccc3)cc(-c3nc(-c4ccccc4)nc(-c4ccc(-c5cccc6c5sc5ccccc56)c5oc6ccccc6c45)n3)c2)cc1] | ¹H NMR (500 MHz, CDCl₃): δ 9.04(d, 2H), 8.8(d, 1H), 8.71(d, 1H), 8.55(d, 1H), 8.28(d, 1H), 8.23(d, 1H), 8.08(d, 1H), 8.00(d, 1H), 7.84~7.83(m, 2H), 7.80~7.77(m, 4H), 7.70~7.57(m, 6H), 7.52~7.43(, 8H), 7.43(dd, 2H) ppm. |
| 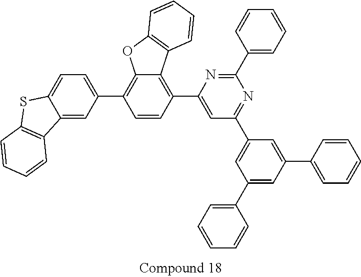 [SMILES: c1ccc(-c2cc(-c3ccccc3)cc(-c3cc(-c4ccc(-c5ccc6sc7ccccc7c6c5)c5oc6ccccc6c45)nc(-c4ccccc4)n3)c2)cc1] | ¹H NMR (500 MHz, CDCl₃): δ 8.80(s, 2H), 8.74(s, 1H), 8.55(s, 2H), 8.34~8.29(m, 1H), 8.23~8.16(m, 2H), 8.08(s, 2H), 8.02(s, 1H), 7.96~7.91(m, 1H), 7.87(s,2H), 7.80(d, 4H), 7.70(dd, 1H), 7.60~7.50(m, 9H), 7.50~7.44(m, 2H), 7.29~7.21(m, 2H) ppm. |
| 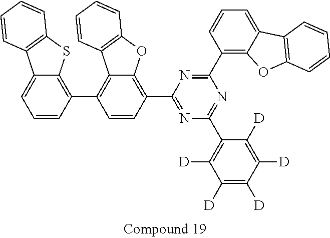 [SMILES: [2H]c1c([2H])c([2H])c(-c2nc(-c3cccc4c3oc3ccccc34)nc(-c3ccc(-c4cccc5c4sc4ccccc45)c4c3oc3ccccc34)n2)c([2H])c1[2H]] | ¹H NMR (500 MHz, CDCl₃): δ 8.97(d, 1H), 8.91(dd, 1H), 8.31(q, 1H), 8.25(d, 1H), 8.18(dd, 1H), 8.0(d, 1H), 7.73-7.77(m, 3H), 7.66-7.69(m, 3H), 7.49-7.57(m, 3H), 7.38-7.43(m, 3H), 6.99-7.02(m, 2H) ppm. |
| 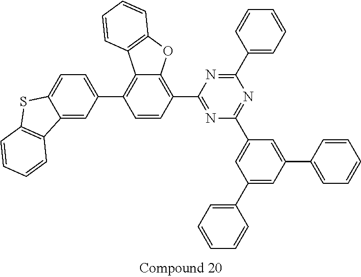 [SMILES: c1ccc(-c2cc(-c3ccccc3)cc(-c3nc(-c4ccccc4)nc(-c4ccc(-c5ccc6sc7ccccc7c6c5)c5c4oc4ccccc45)n3)c2)cc1] | ¹H NMR (500 MHz, CDCl₃): δ 9.09(d, 2H), 8.84(dd, 3H), 8.42(s, 1H), 8.16(d, 1H), 7.99-8.03(m, 2H), 7.93(d, 1H), 7.83(d, 4H), 7.71(d, 1H), 7.67(d, 1H), 7.42-7.60(m, 14H), 7.08(t, 1H) ppm. |

Modifications of the Claimed Novel Compounds

In addition to Compounds 1 to 20, one person skilled in the art can react any Intermediate A, i.e., the foresaid Intermediate An, or the like, with any Reactant Bn or the like through a reaction mechanism similar to Scheme I or Scheme II to synthesize other desired claimed novel compounds.

Preparation of OLED Devices

A glass substrate coated with an ITO layer (hereinafter referred to as ITO substrate) in a thickness of 1500 Å was placed in distilled water containing a detergent dissolved therein, and was ultrasonically washed. The detergent was a product manufactured by Fischer Co., and the distilled water was distilled water filtered twice through a filter (Millipore Co.). After the ITO layer had been washed for 30 minutes, it was ultrasonically washed twice with distilled water for 10 minutes. After the completion of washing, the glass substrate was ultrasonically washed with isopropyl alcohol, acetone and methanol solvents and then dried, after which it was transported to a plasma cleaner. Then the substrate was cleaned with oxygen plasma for 5 minutes, and then transferred to a vacuum evaporator.

After that, various organic materials and metal materials were sequentially deposited on the ITO substrate to obtain the OLED device of Examples 1 to 51 and Comparative Examples 1 to 12. The vacuum degree during the deposition was maintained at 1×10⁻⁶ to 3×10⁻⁷ torr. Herein, the ITO substrate was deposited with a hole injection layer (HIL), a first hole transporting layer (HTL-1), a second hole transporting layer (HTL-2), a blue/green/red emission layer (BEL/GEL/REL), an electron transporting layer (ETL), an electron injection layer (EIL), and a cathode (Cthd).

Herein, HI-D was a dopant with HI for forming HIL; HT1 was a material for forming HTL-1, and B-HT2/G-HT2/R-HT2 were respectively materials for forming blue, green and red HTL-2; conventional ET and novel compounds of the present invention were materials for forming ETL; Liq was a dopant for forming ETL and was a material for forming EIL. RH/GH/BH were host materials for forming REL/GEL/BEL, and RD/GD/BD were dopants for forming REL/GEL/BEL. The main difference of the OLEDs between Examples and Comparative Examples was that the ETL of the OLED in the following comparative examples was made of ET1 or ET2 but the ETL of OLED in the following examples was made of the novel compounds of the present invention listed in Table 7. The detailed chemical structures of foresaid commercial materials were listed in Table 9.

TABLE 9
The chemical structures of commercial materials, ET1 and ET2 for
OLED devices.

| | |
|---|---|
| 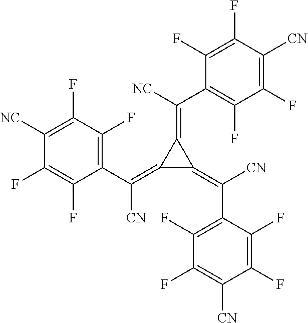 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] | HI-D |
| 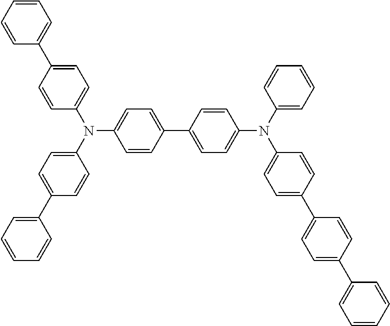 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1] | HI |
| 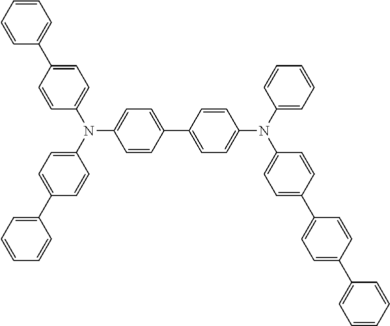 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1] | HT1 |
| 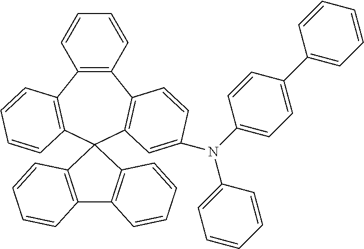 [SMILES: c1ccc(-c2ccc(N(c3ccccc3)c3ccc4c(c3)C3(c5ccccc5-c5ccccc5-4)c4ccccc4-c4ccccc43)cc2)cc1] | B-HT2 |
| 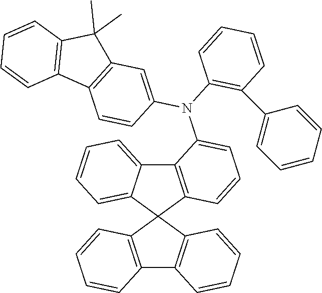 [SMILES: CC1(C)c2ccccc2-c2ccc(N(c3ccccc3-c3ccccc3)c3cccc4c3-c3ccccc3C43c4ccccc4-c4ccccc43)cc21] | G-HT2/R-HT2 |
| 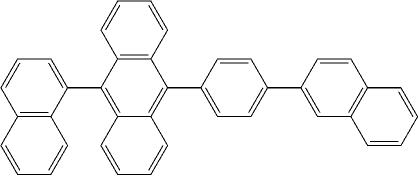 [SMILES: c1ccc2cc(-c3ccc(-c4c5ccccc5c(-c5cccc6ccccc56)c5ccccc45)cc3)ccc2c1] | BH |
| 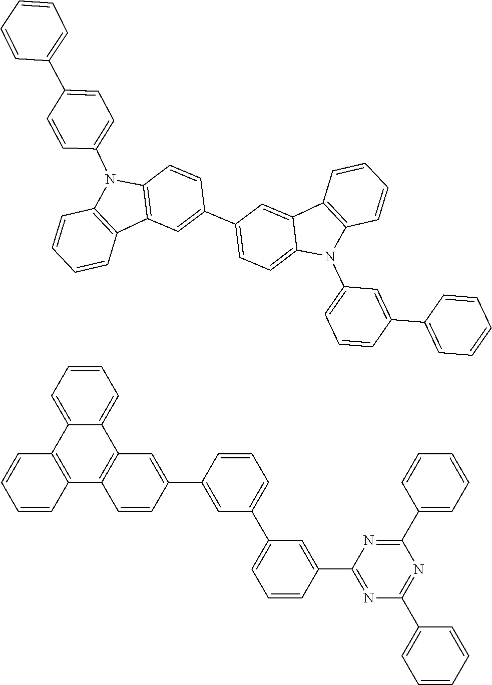 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2nc(-c3ccccc3)nc(-c3cccc(-c4cccc(-c5ccc6c7ccccc7c7ccccc7c6c5)c4)c3)n2)cc1] | GH (1:1) |
| 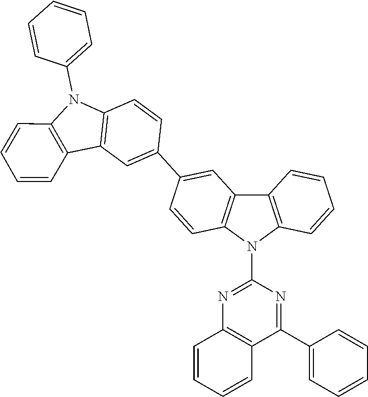 [SMILES: c1ccc(-c2nc(-n3c4ccccc4c4cc(-c5ccc6c(c5)c5ccccc5n6-c5ccccc5)ccc43)nc3ccccc23)cc1] | RH |
| 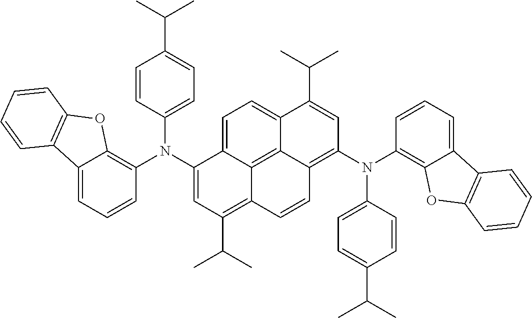 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1] | BD |
| 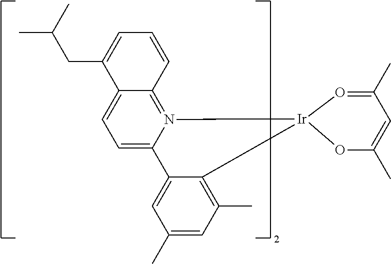 | RD |
| 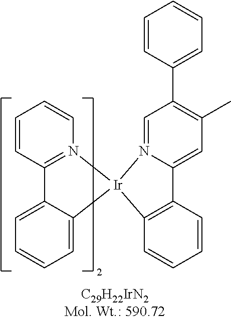 | GD |
| 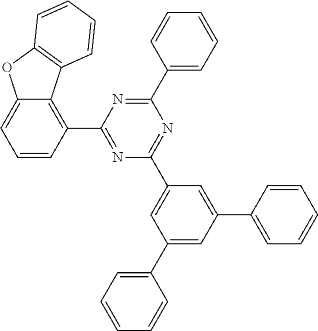 [SMILES: c1ccc(-c2cc(-c3ccccc3)cc(-c3nc(-c4ccccc4)nc(-c4cccc5oc6ccccc6c45)n3)c2)cc1] | ET1 |
| 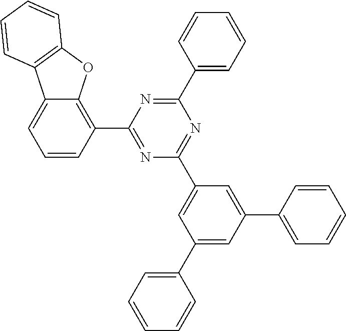 [SMILES: c1ccc(-c2cc(-c3ccccc3)cc(-c3nc(-c4ccccc4)nc(-c4cccc5c4oc4ccccc45)n3)c2)cc1] | ET2 |
| 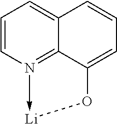 | ETD (Liq) |

OLEDs with a Single Electron Transport Layer

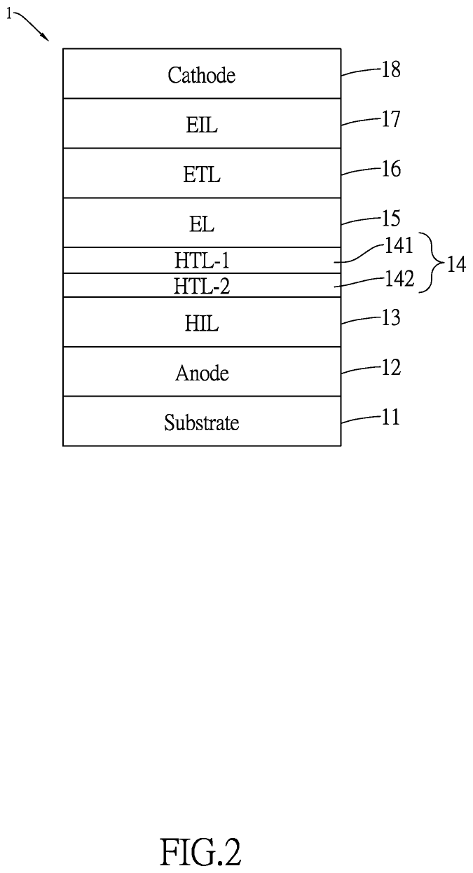

In OLEDs with a single electron transport layer, various organic materials and metal materials were sequentially deposited on the ITO substrate to obtain the OLED device of Examples 1 to 32 and Comparative Examples 1 to 6. Herein, as shown in FIG. 2, OLED 1 may have a structure of a substrate 11, an anode 12, a HIL 13, a HTL 14 containing a HTL-1 141 and a HTL-2 142, an EL 15, an ETL 16, an EIL 17, and a cathode 18 stacked in sequence.

Preparation of Blue OLED Devices

To prepare the blue OLED device, multiple organic layers were respectively deposited on the ITO substrate according to the sequence as listed in Table 10, and the materials and the thicknesses of the organic layers in blue OLED devices were also listed in Table 10.

TABLE 10
Coating sequence, materials and thickness of the
layers in the blue OLED devices.

| Coating | | | |
|---|---|---|---|
| Sequence | Layer | Material | Thickness |
| 1 | HIL | HI doped with 3.0 wt % | 100 Å |
| | | of HI-D | |
| 2 | HTL-1 | HT1 | 850 Å |
| 3 | HTL-2 | B-HT2 | 100 Å |
| 4 | BEL | BH doped with 3.5 wt % | 250 Å |
| | | of BD | |
| 5 | ETL | ET1/ET2/novel claimed | 350 Å |
| | | compounds doped with | |
| | | 35.0 wt % of Liq | |
| 6 | EIL | Liq | 15 Å |
| 7 | Cthd | A1 | 1500 Å |

Preparation of Green OLED Devices

To prepare the green OLED device, multiple organic layers were respectively deposited on the ITO substrate according to the sequence as listed in Table 11, and the materials and the thicknesses of the organic layers in green OLED devices were also listed in Table 11.

TABLE 11
Coating sequence, materials and thickness of the
layers in the green OLED devices.

| Coating | | | |
|---|---|---|---|
| Sequence | Layer | Material | Thickness |
| 1 | HIL | HI doped with 3.0 wt % | 100 Å |
| | | of HI-D | |
| 2 | HTL-1 | HT1 | 1400 Å |
| 3 | HTL-2 | G-HT2 | 100 Å |
| 4 | GEL | GH doped with 10.0 wt % | 400 Å |
| | | of GD | |
| 5 | ETL | ET1/ET2/novel compounds | 350 Å |
| | | doped with 35.0 wt % of Liq | |
| 6 | EIL | Liq | 15 Å |
| 7 | Cthd | A1 | 1500 Å |

Preparation of Red OLED Devices

To prepare the red OLED device, multiple organic layers were respectively deposited on the ITO substrate according to the sequence as listed in Table 12, and the materials and the thicknesses of the organic layers in red OLED devices were also listed in Table 12.

TABLE 12
Coating sequence, materials and thickness of the
layers in the red OLED devices.

| Coating | | | |
|---|---|---|---|
| Sequence | Layer | Material | Thickness |
| 1 | HIL | HI doped with 3.0 wt % | 100 Å |
| | | of HI-D | |
| 2 | HTL-1 | HT1 | 2200 Å |
| 3 | HTL-2 | R-HT2 | 100 Å |
| 4 | REL | RH doped with 3.5 wt % | 300 Å |
| | | of RD | |
| 5 | ETL | ET1/ET2/novel compounds | 350 Å |
| | | doped with 35.0 wt % of Liq | |
| 6 | EIL | Liq | 15 Å |
| 7 | Cthd | A1 | 1500 Å |

Performance of OLED Devices

To evaluate the performance of OLED devices, the OLED devices were measured by PR650 as photometer and Keithley 2400 as power supply. Color coordinates (x,y) were determined according to the CIE chromaticity scale (Commission Internationale de L'Eclairage, 1931).

Measurement of Lifespan

The evaluation of lifespan was measured by OLED life time test system (Chroma model 58131). Measurements of lifespan for blue, green, and red OLEDs were respectively performed according to the following circumstances.

For blue OLEDs, the evaluation of lifespan (T85) was defined as a period taken for luminance reduction to 85% of the initial luminance at 2000 nits. The results of blue OLEDs were shown in Table 13.

For green OLEDs, the evaluation of lifespan (T95) was defined as a period taken for luminance reduction to 95% of the initial luminance at 7000 nits. The results of green OLEDs were shown in Table 14.

For red OLEDs, the evaluation of lifespan (T90) was defined as a period taken for luminance reduction to 90% of the initial luminance at 6000 nits. The results of red OLEDs were shown in Table 15.

The materials of ETL, datas of CIE and lifespan of Examples 1 to 32 and Comparative Examples 1 to 6 were listed in Table 13, Table 14 and Table 15.

TABLE 13
Materials of ETL, CIEs and lifespan of blue
OLED devices of Examples 1 to 15 and
Comparative Examples 1 to 2.

| Example | Material of | | Lifespan |
|---|---|---|---|
| No. | ETL | CIE(x, y) | (T85) (hrs) |
| E1 | Compound 1 | (0.130, 0.151) | 469 |
| E2 | Compound 2 | (0.130, 0.150) | 407 |
| E3 | Compound 3 | (0.131, 0.155) | 208 |
| E4 | Compound 5 | (0.130, 0.158) | 356 |
| E5 | Compound 6 | (0.131, 0.154) | 269 |
| E6 | Compound 9 | (0.130, 0.152) | 254 |
| E7 | Compound 11 | (0.130, 0.151) | 185 |
| E8 | Compound 16 | (0.131, 0.145) | 255 |
| E9 | Compound 17 | (0.131, 0.147) | 530 |
| E10 | Compound 18 | (0.131, 0.147) | 212 |
| E11 | Compound 8 | (0.131, 0.145) | 317 |
| E12 | Compound 13 | (0.131, 0.147) | 288 |
| E13 | Compound 14 | (0.131, 0.145) | 296 |
| E14 | Compound 15 | (0.130, 0.151) | 200 |
| E15 | Compound 20 | (0.131, 0.144) | 234 |
| C1 | ET1 | (0.131, 0.148) | 52 |
| C2 | ET2 | (0.131, 0.147) | 164 |

TABLE 14
Materials of ETL, CIEs and lifespan of green
OLED devices of Examples 16 to 26 and
Comparative Examples 3 to 4.

| Example | Material of | | Lifespan |
|---|---|---|---|
| No. | ETL | CIE(x, y) | (T95) (hrs) |
| E16 | Compound 2 | (0.323, 0.628) | 342 |
| E17 | Compound 9 | (0.324, 0.630) | 279 |
| E18 | Compound 11 | (0.325, 0.629) | 203 |
| E19 | Compound 16 | (0.331, 0.626) | 233 |
| E20 | Compound 17 | (0.333, 0.625) | 255 |
| E21 | Compound 18 | (0.329, 0.627) | 310 |
| E22 | Compound 13 | (0.330, 0.626) | 253 |
| E23 | Compound 14 | (0.326, 0.629) | 239 |
| E24 | Compound 15 | (0.321, 0.632) | 237 |
| E25 | Compound 19 | (0.321, 0.630) | 186 |
| E26 | Compound 20 | (0.320, 0.631) | 267 |
| C3 | ET1 | (0.329, 0.627) | 97 |
| C4 | ET2 | (0.322, 0.631) | 172 |

TABLE 15
Materials of ETL, CIEs and lifespan of red
OLED devices of Examples 27 to 32 and
Comparative Examples 5 to 6.

| Example | Material of | | Lifespan |
|---|---|---|---|
| No. | ETL | CIE(x, y) | (T90) (hrs) |
| E27 | Compound 1 | (0.661, 0.337) | 330 |
| E28 | Compound 2 | (0.660, 0.336) | 315 |
| E29 | Compound 3 | (0.661, 0.337) | 341 |
| E30 | Compound 12 | (0.661, 0.337) | 326 |
| E31 | Compound 17 | (0.665, 0.333) | 310 |
| E32 | Compound 18 | (0.660, 0.338) | 309 |
| C5 | ET1 | (0.663, 0.336) | 245 |
| C6 | ET2 | (0.663, 0.335) | 301 |

As shown in Tables 13 to 15, in comparison with the conventional ET materials (i.e., ET1 and ET2), adopting the novel compounds of the present invention as the electron transport material can effectively prolong lifespan of the blue, green, or red OLEDs with a single electron transport layer.

OLEDs with Double Electron Transport Layer

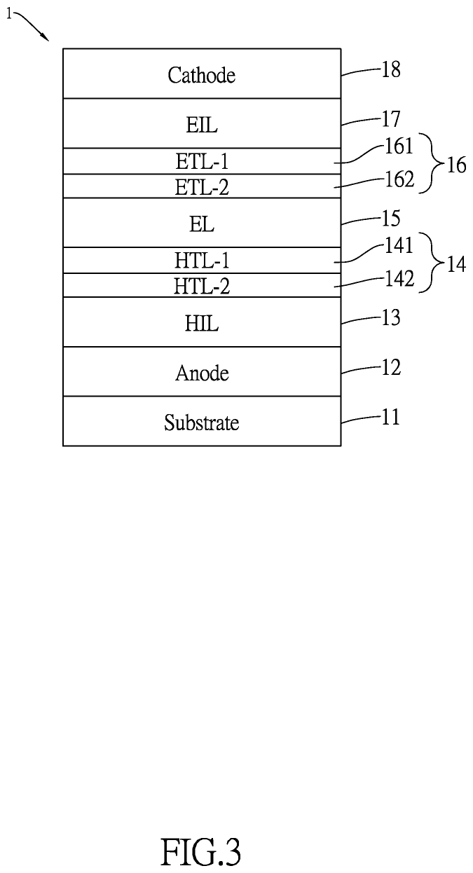

Like OLEDs with a single electron transport layer, various organic materials and metal materials were sequentially deposited on the ITO substrate to obtain the OLED device of Examples 33 to 51 and Comparative Examples 7 to 12. Herein, as shown in FIG. 3, OLED 1 may have a structure of a substrate 11, an anode 12, a HIL 13, a HTL 14 containing a HTL-1 141 and a HTL-2 142, an EL 15, an ETL 16 containing a first electron transport layer (ETL-1) 161 and a second electron transport layer (ETL-2) 162, an EIL 17, and a cathode 18 stacked in sequence.

Preparation of Blue OLED Devices

To prepare the blue OLED device, multiple organic layers were respectively deposited on the ITO substrate according to the sequence as listed in Table 16, and the materials and the thicknesses of the organic layers in blue OLED devices were also listed in Table 16.

TABLE 16
Coating sequence, materials and thickness of the layers in the blue
OLED devices.

| Coating | | | | |
|---|---|---|---|---|
| Sequence | Layer | Material | Thickness | |
| 1 | HIL | HI doped with 3.0 wt % of HI-D | 100 | Å |
| 2 | HTL-1 | HT1 | 850 | Å |
| 3 | HTL-2 | B-HT2 | 100 | Å |
| 4 | BEL | BH doped with 3.5 wt % of BD | 250 | Å |
| 5 | ETL-1 | 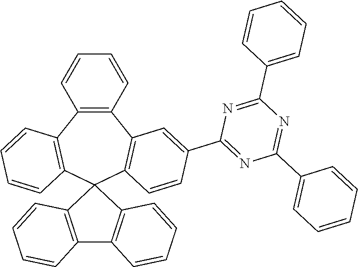 [SMILES: c1ccc(-c2nc(-c3ccccc3)nc(-c3ccc4c(c3)-c3ccccc3-c3ccccc3C43c4ccccc4-c4ccccc43)n2)cc1] | 100 | Å |
| 6 | ETL-2 | ET1/ET2/novel compounds doped with | 250 | Å |
| | | 35.0 wt % of Liq | | |
| 7 | EIL | Liq | 15 | Å |
| 8 | Cthd | Al | 1500 | Å |

Preparation of Green OLED Devices

To prepare the green OLED device, multiple organic layers were respectively deposited on the ITO substrate according to the sequence as listed in Table 17, and the materials and the thicknesses of the organic layers in green OLED devices were also listed in Table 17.

TABLE 17
Coating sequence, materials and thickness of the layers in the green
OLED devices.

| Coating | | | | |
|---|---|---|---|---|
| Sequence | Layer | Material | Thickness | |
| 1 | HIL | HI doped with 3.0 wt % of HI-D | 100 | Å |
| 2 | HTL-1 | HT1 | 1400 | Å |
| 3 | HTL-2 | G-HT2 | 100 | Å |
| 4 | GEL | GH doped with 3.5 wt % of GD | 400 | Å |
| 5 | ETL-1 | 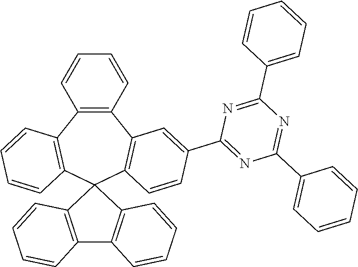 [SMILES: c1ccc(-c2nc(-c3ccccc3)nc(-c3ccc4c(c3)-c3ccccc3-c3ccccc3C43c4ccccc4-c4ccccc43)n2)cc1] | 100 | Å |
| 6 | ETL-2 | ET1/ET2/novel compounds doped with | 250 | Å |
| | | 35.0 wt % of Liq | | |
| 7 | EIL | Liq | 15 | Å |
| 8 | Cthd | Al | 1500 | Å |

Preparation of Red OLED Devices

To prepare the red OLED device, multiple organic layers were respectively deposited on the ITO substrate according to the sequence as listed in Table 18, and the materials and the thicknesses of the organic layers in red OLED devices were also listed in Table 18.

TABLE 18
Coating sequence, materials and thickness of the layers in the red
OLED devices.

| Coating | | | | |
|---|---|---|---|---|
| Sequence | Layer | Material | Thickness | |
| 1 | HIL | HI doped with 3.0 wt % of HI-D | 100 | Å |
| 2 | HTL-1 | HT1 | 2200 | Å |
| 3 | HTL-2 | R-HT2 | 100 | Å |
| 4 | REL | RH doped with 3.5 wt % of RD | 300 | Å |
| 5 | ETL-1 | 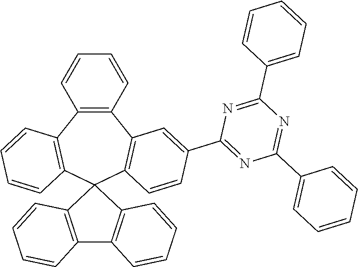 [SMILES: c1ccc(-c2nc(-c3ccccc3)nc(-c3ccc4c(c3)-c3ccccc3-c3ccccc3C43c4ccccc4-c4ccccc43)n2)cc1] | 100 | Å |
| 6 | ETL-2 | ET1/ET2/novel compounds doped with | 250 | Å |
| | | 35.0 wt % of Liq | | |
| 7 | EIL | Liq | 15 | Å |
| 8 | Cthd | Al | 1500 | Å |

Performance of OLED Devices

The evaluation of the performance of OLED devices with double electron transport layers was performed in a same manner with OLEDs devices with a single electron transport layer.

Measurement of Lifespan

The evaluation of lifespan OLED devices with double electron transport layers was also measured. The materials of ETL-2, data of CIE and lifespan of Examples 33 to 51 and Comparative Examples 7 to 12 were listed in Table 19, Table 20 and Table 21.

TABLE 19
Materials of ETL-2, CIEs and lifespan of blue
OLED devices of Examples 33 to 41 and
Comparative Examples 7 to 8.

| Example | Material of | | Lifespan |
|---|---|---|---|
| No. | ETL-2 | CIE(x, y) | (T85) (hrs) |
| E33 | Compound 1 | (0.132, 0.134) | 239 |
| E34 | Compound 5 | (0.132, 0.135) | 267 |
| E35 | Compound 6 | (0.131, 0.140) | 206 |
| E36 | Compound 11 | (0.133, 0.131) | 195 |
| E37 | Compound 13 | (0.131, 0.138) | 228 |
| E38 | Compound 14 | (0.131, 0.139) | 244 |
| E39 | Compound 15 | (0.132, 0.135) | 243 |
| E40 | Compound 17 | (0.133, 0.134) | 470 |
| E41 | Compound 8 | (0.133, 0.130) | 265 |
| C7 | ET1 | (0.133, 0.128) | 78 |
| C8 | ET2 | (0.132, 0.130) | 113 |

TABLE 20
Materials of ETL-2, CIEs and lifespan of green
OLED devices of Examples 42 to 45 and
Comparative Examples 9 to 10.

| Example | Material of | | Lifespan |
|---|---|---|---|
| No. | ETL-2 | CIE(x, y) | (T95) (hrs) |
| E42 | Compound 1 | (0.349, 0.624) | 396 |
| E43 | Compound 13 | (0.357, 0.619) | 385 |
| E44 | Compound 14 | (0.352, 0.623) | 373 |
| E45 | Compound 17 | (0.352, 0.623) | 374 |
| C9 | ET1 | (0.357, 0.619) | 145 |
| C10 | ET2 | (0.350, 0.624) | 353 |

TABLE 21
Materials of ETL-2, CIEs and lifespan of red
OLED devices of Examples 46 to 51 and
Comparative Examples 11 to 12.

| Example | Material of | | Lifespan |
|---|---|---|---|
| No. | ETL-2 | CIE(x, y) | (T90) (hrs) |
| E46 | Compound 1 | (0.682, 0.317) | 444 |
| E47 | Compound 5 | (0.682, 0.317) | 493 |
| E48 | Compound 13 | (0.682, 0.315) | 424 |
| E49 | Compound 14 | (0.682, 0.316) | 460 |
| E50 | Compound 15 | (0.682, 0.316) | 357 |
| E51 | Compound 17 | (0.682, 0.316) | 447 |
| C11 | ET1 | (0.682, 0.315) | 270 |
| C12 | ET2 | (0.681, 0.317) | 350 |

As shown in Tables 19 to 21, in comparison with the conventional ET materials (i.e., ET1 and ET2), adopting the novel compounds of the present invention as the electron transport material of the second electron transport layer also can effectively prolong lifespan of the blue, green, or red OLEDs with double electron transport layers.

Measurement of Driving Voltage

In addition to lifespan of OLEDs, the evaluation of driving voltage of OLED devices with double electron transport layers was also performed. The materials of ETL-2, data of CIE and driving voltage of Examples 33, 36, 40 to 42, 45 to 47 and 51, and Comparative Examples 7 to 12 were listed in Table 22.

TABLE 22
Materials of ETL-2, CIEs and driving voltage of
OLED devices of Examples 33, 36, 40 to 42, 45 to
47 and 51, and Comparative Examples 7 to 12.

| Example | Material of | | Voltage |
|---|---|---|---|
| No. | ETL-2 | CIE(x, y) | (V) |
| E33 | Compound 1 | B (0.132, 0.134) | 3.55 |
| E36 | Compound 11 | B (0.132, 0.135) | 3.53 |
| E40 | Compound 17 | B (0.131, 0.140) | 3.58 |
| E41 | Compound 8 | B (0.133, 0.131) | 3.44 |
| C7 | ET1 | B (0.133, 0.128) | 4.58 |
| C8 | ET2 | B (0.132, 0.130) | 3.64 |
| E42 | Compound 1 | G (0.349, 0.624) | 3.10 |
| E45 | Compound 17 | G (0.352, 0.623) | 3.05 |
| C9 | ET1 | G (0.357, 0.619) | 4.29 |
| C10 | ET2 | G (0.350, 0.624) | 3.27 |
| E46 | Compound 1 | R (0.682, 0.317) | 3.65 |
| E47 | Compound 5 | R (0.682, 0.317) | 3.69 |
| E51 | Compound 17 | R (0.682, 0.316) | 3.6 |
| C11 | ET1 | R (0.682, 0.315) | 4.91 |
| C12 | ET2 | R (0.681, 0.317) | 3.77 |

As shown in Table 22, in comparison with the conventional ET materials (i.e., ET1 and ET2), adopting the novel compounds of the present invention as the electron transport material of the second electron transport layer can additionally reduce driving voltage of the blue, green, or red OLEDs with double electron transport layers.

In brief, regardless of in OLED devices with single or double electron transport layers, in comparison with the conventional ET materials, adopting the novel compounds of the present invention as the electron transport material can effectively prolong lifespan of the blue, green, or red OLEDs. Moreover, in OLED devices with double electron transport layers, adopting the novel compounds of the present invention as the electron transport material can further reduce the driving voltage of the blue, green, or red OLEDs.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A compound represented by the following Formula (I): 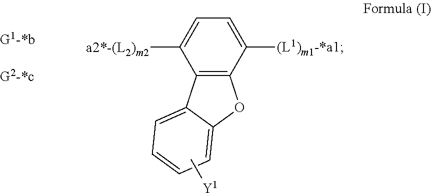
wherein *a1, a2*, *b, and *c represent bonding sites, *b is bonded to one of *a1 and a2*, and *c is bonded to the other of *a1 and a2*;
wherein G¹-*b is represented by 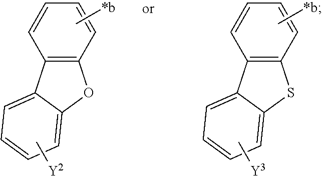
wherein G² is selected from the group consisting of: 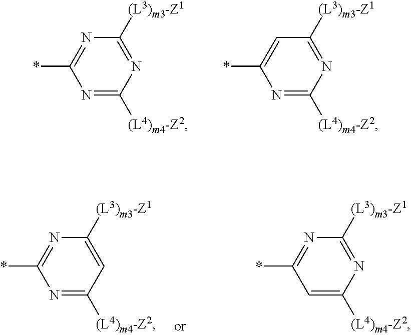
wherein Z¹ and Z² are each independently selected from the group consisting of: a substituted aryl group having 6 to 60 ring carbon atoms, an unsubstituted aryl group having 6 to 60 ring carbon atoms, a substituted heteroaryl group having 3 to 60 ring carbon atoms, and an unsubstituted heteroaryl group having 3 to 60 ring carbon atoms;
wherein m1 to m4 are each independently an integer 0 or 1, and m1 to m4 are the same or different;
wherein L¹ to L⁴ are each independently an arylene group having 6 to 60 ring carbon atoms, and L¹ to L⁴ are the same or different;
wherein Y¹ to Y³ are each independently selected from the group consisting of: a hydrogen atom, a deuterium atom, an alkyl group having 1 to 12 carbon atoms, and an aryl group having 6 to 30 ring carbon atoms, and Y¹ to Y³ are the same or different.

2. The compound as claimed in claim 1, wherein the compound is represented by any one of the following formulae (I-I) to (I-XVI):

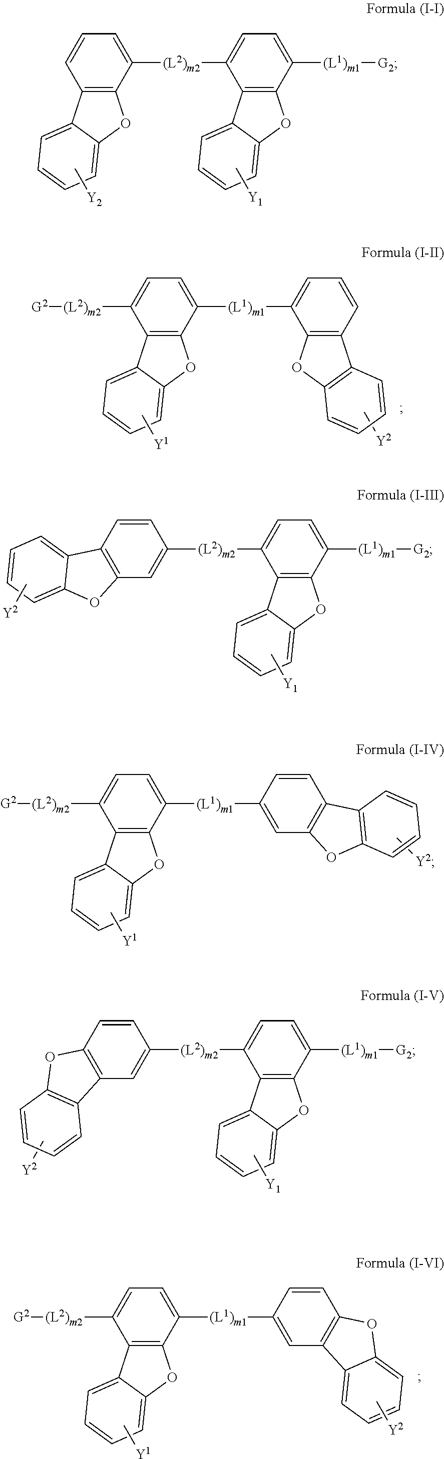

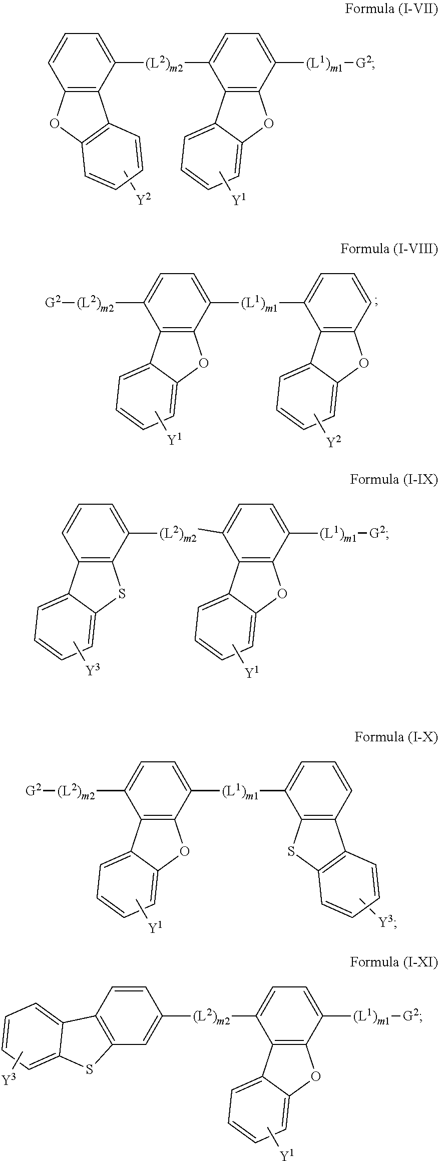

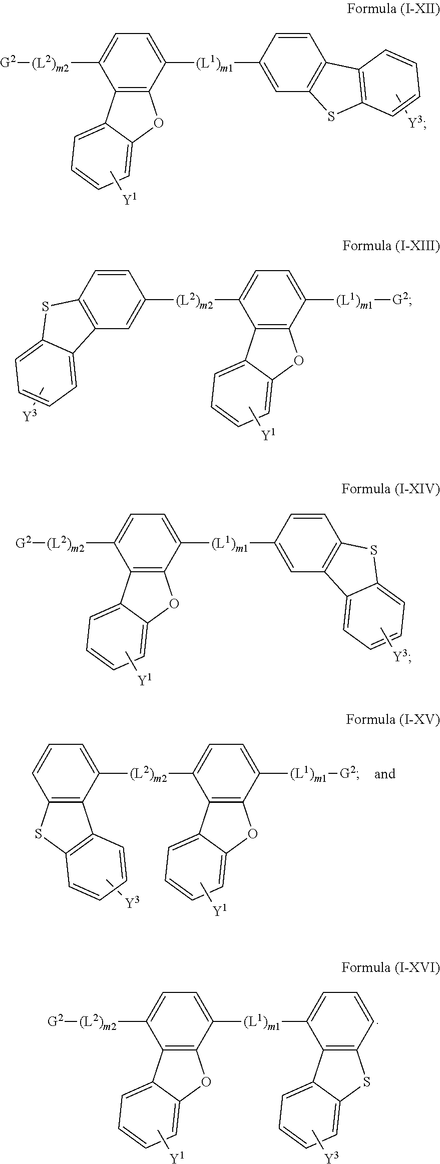

3. The compound as claimed in claim 1, wherein Z¹ and Z² are each independently selected from the group consisting of: 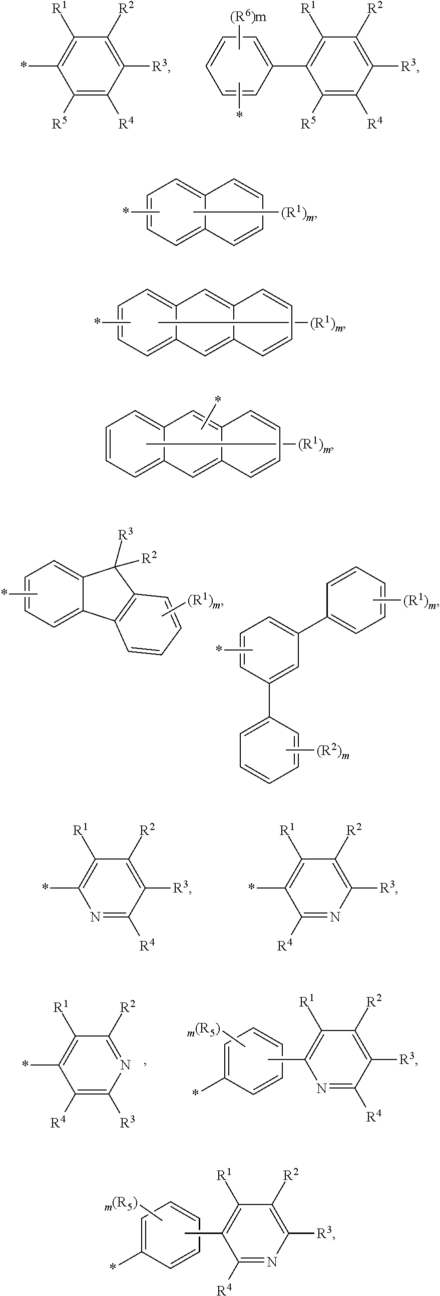 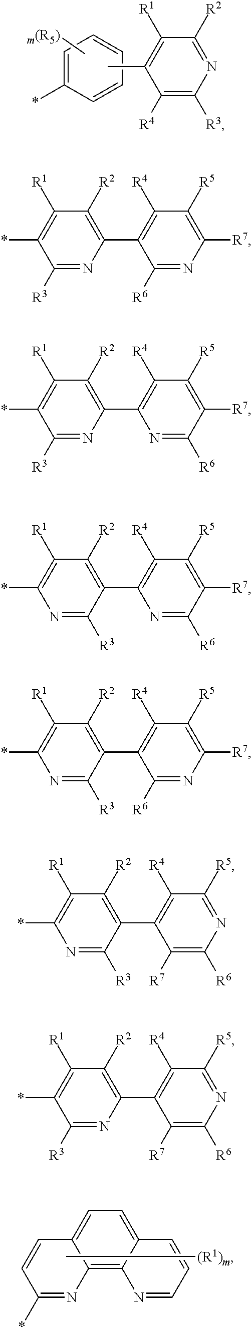 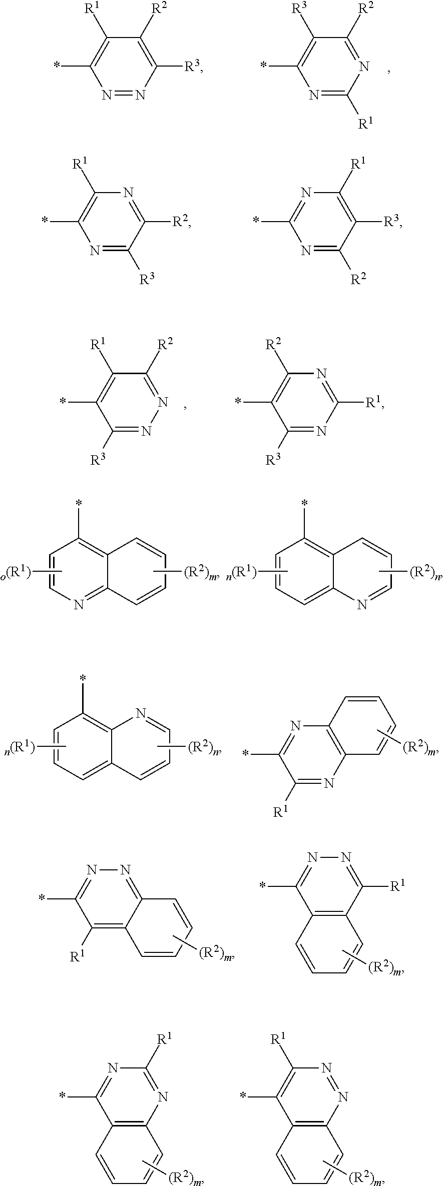 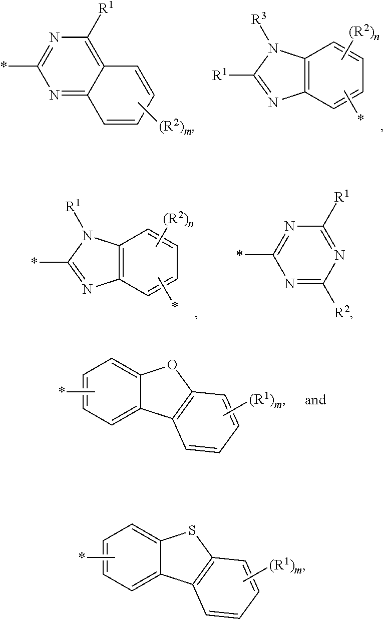
wherein R¹ to R⁷ are each independently selected from the group consisting of: a hydrogen atom, a deuterium atom, a halogen group, a cyano group, a nitro group, a trifluoromethyl group, an unsubstituted alkyl group having 1 to 12 carbon atoms, an alkyl group having 1 to 12 carbon atoms substituted with a substituent, an unsubstituted alkenyl group having 2 to 12 carbon atoms, an alkenyl group having 2 to 12 carbon atoms substituted with a substituent, an unsubstituted alkynyl group having 2 to 12 carbon atoms, an alkynyl group having 2 to 12 carbon atoms substituted with a substituent, an unsubstituted aryl group having 6 to 30 ring carbon atoms, an aryl group having 6 to 30 ring carbon atoms substituted with a substituent, an unsubstituted heteroaryl group having 3 to 30 ring carbon atoms, and a heteroaryl group having 3 to 30 ring carbon atoms substituted with a substituent, wherein the substituent is selected from the group consisting of: a deuterium atom, a halogen group, a cyano group, a nitro group, and a trifluoromethyl group;
wherein m is an integer from 1 to 4, n is an integer from 1 to 3, and o is an integer 1 or 2.

4. The compound as claimed in claim 1, wherein Z¹ is selected from the group consisting of: 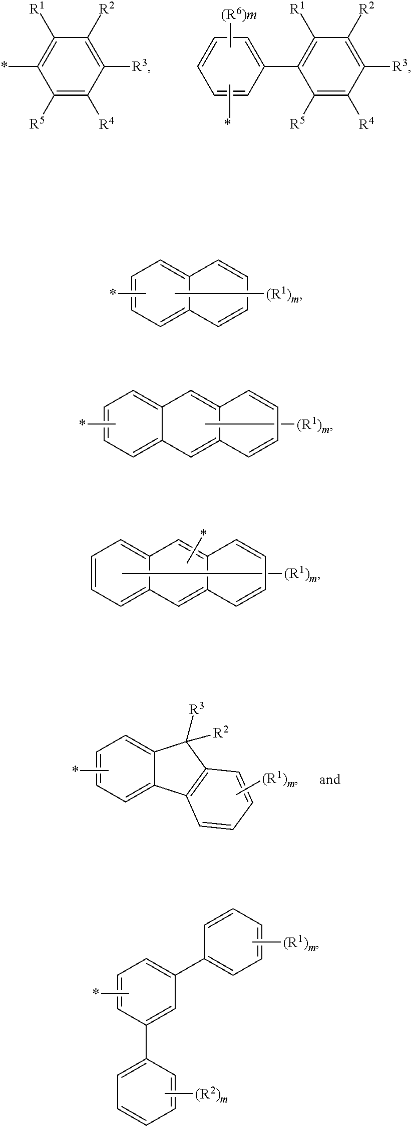
Z² is selected from the group consisting of: 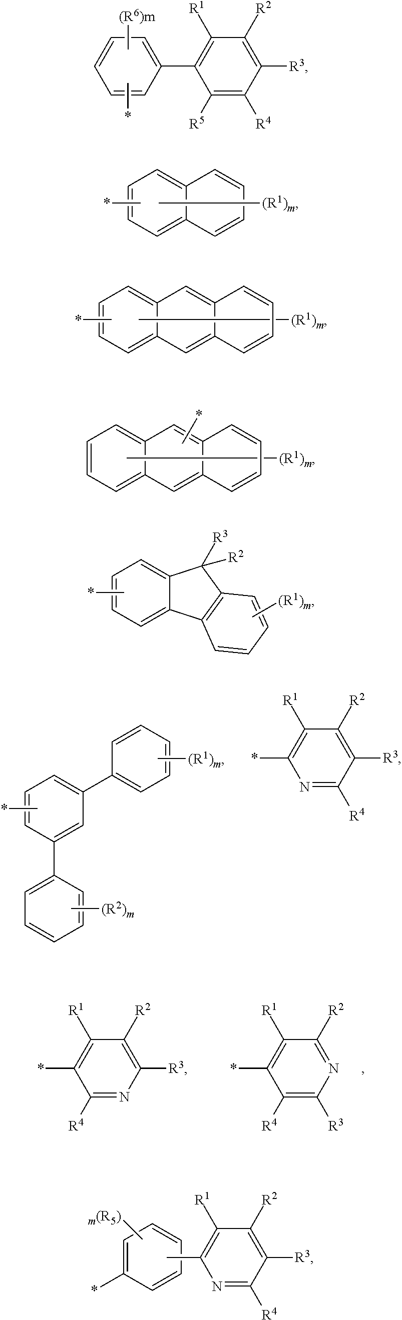 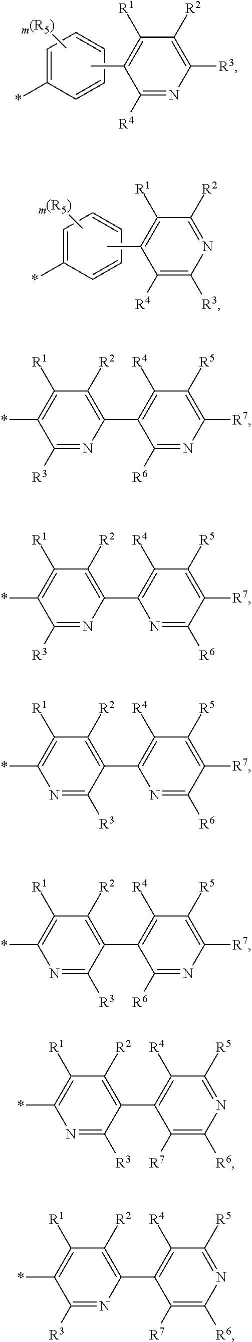 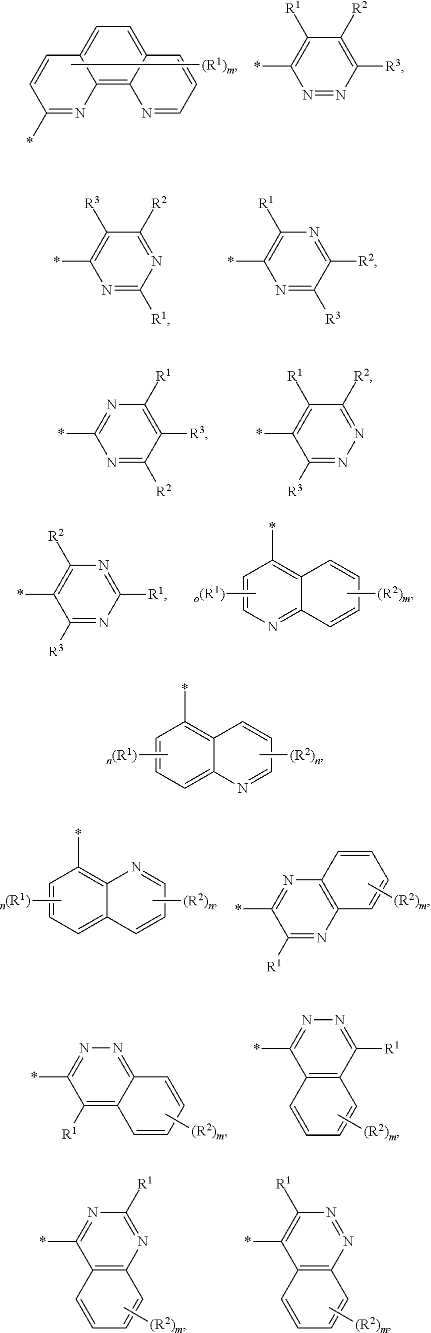 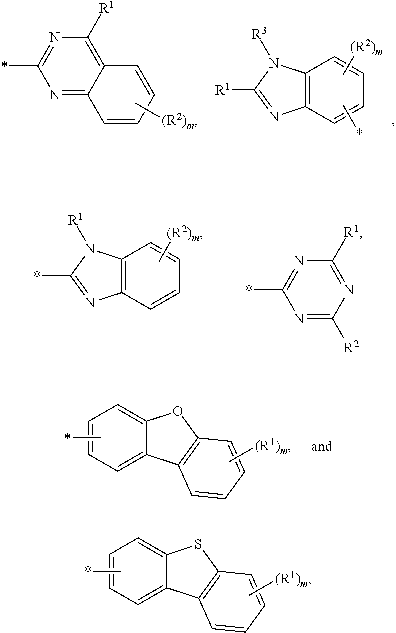
wherein R¹ to R⁷ are each independently selected from the group consisting of: a hydrogen atom, a deuterium atom, a halogen group, a cyano group, a nitro group, a trifluoromethyl group, an unsubstituted alkyl group having 1 to 12 carbon atoms, an alkyl group having 1 to 12 carbon atoms substituted with a substituent, an unsubstituted alkenyl group having 2 to 12 carbon atoms, an alkenyl group having 2 to 12 carbon atoms substituted with a substituent, an unsubstituted alkynyl group having 2 to 12 carbon atoms, an alkynyl group having 2 to 12 carbon atoms substituted with a substituent, an unsubstituted aryl group having 6 to 30 ring carbon atoms, an aryl group having 6 to 30 ring carbon atoms substituted with a substituent, an unsubstituted heteroaryl group having 3 to 30 ring carbon atoms, and a heteroaryl group having 3 to 30 ring carbon atoms substituted with a substituent, wherein the substituent is selected from the group consisting of: a deuterium atom, a halogen group, a cyano group, a nitro group, and a trifluoromethyl group;
wherein m is an integer from 1 to 4, n is an integer from 1 to 3, and o is an integer 1 or 2.

5. The compound as claimed in claim 1, wherein Z¹ and Z² are each independently selected from the group consisting of: 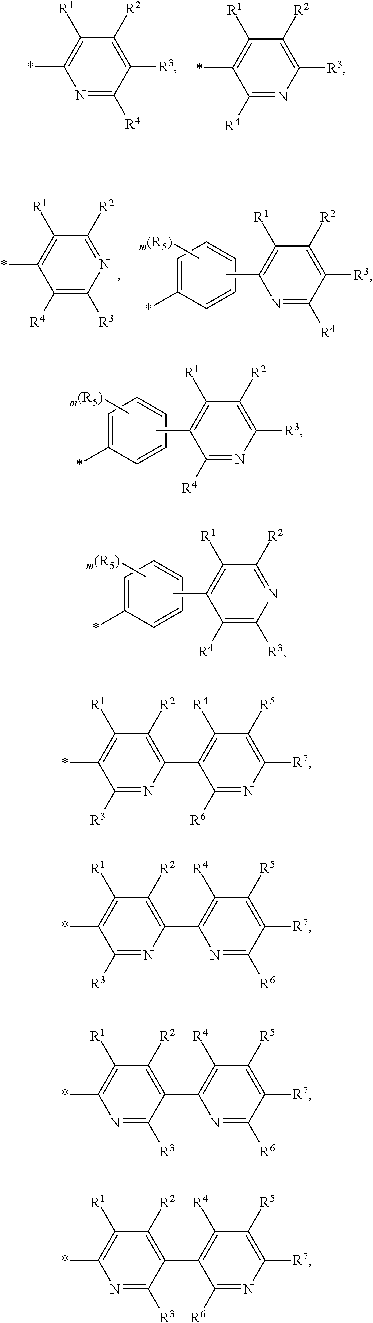 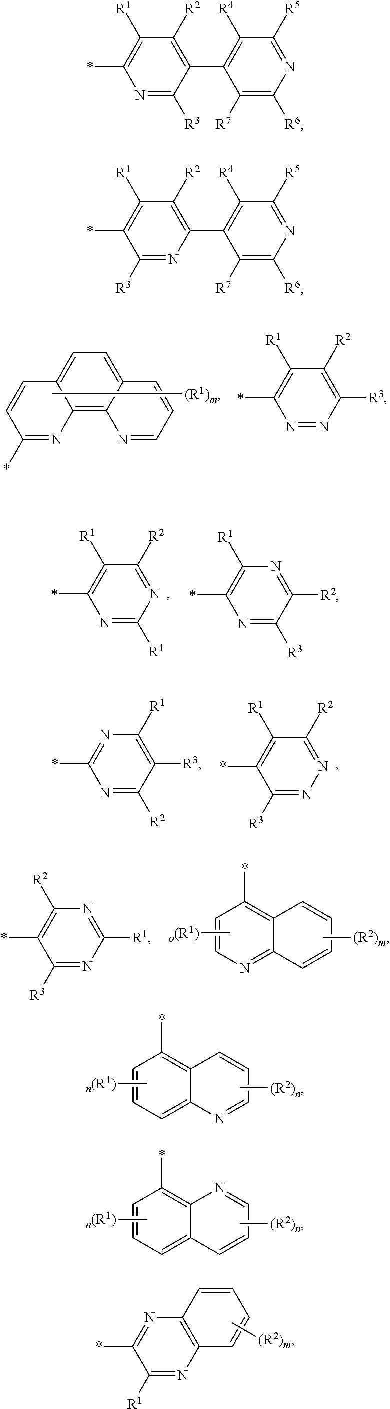 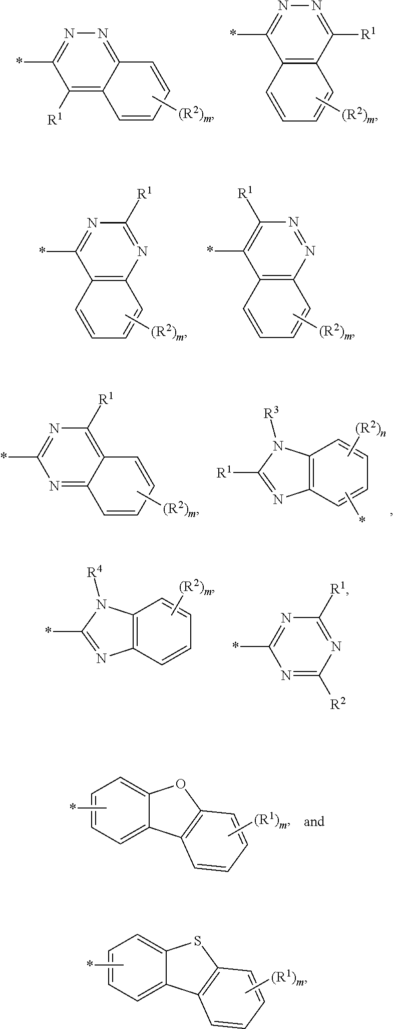
wherein R¹ to R⁷ are each independently selected from the group consisting of: a hydrogen atom, a deuterium atom, a halogen group, a cyano group, a nitro group, a trifluoromethyl group, an unsubstituted alkyl group having 1 to 12 carbon atoms, an alkyl group having 1 to 12 carbon atoms substituted with a substituent, an unsubstituted alkenyl group having 2 to 12 carbon atoms, an alkenyl group having 2 to 12 carbon atoms substituted with a substituent, an unsubstituted alkynyl group having 2 to 12 carbon atoms, an alkynyl group having 2 to 12 carbon atoms substituted with a substituent, an unsubstituted aryl group having 6 to 30 ring carbon atoms, an aryl group having 6 to 30 ring carbon atoms substituted with a substituent, an unsubstituted heteroaryl group having 3 to 30 ring carbon atoms, and a heteroaryl group having 3 to 30 ring carbon atoms substituted with a substituent, wherein the substituent is selected from the group consisting of: a deuterium atom, a halogen group, a cyano group, a nitro group, and a trifluoromethyl group;
wherein m is an integer from 1 to 4, n is an integer from 1 to 3, and o is an integer 1 or 2.

6. The compound as claimed in claim 4, wherein R¹ to R⁷ are each selected from the group consisting of: a hydrogen atom, a deuterium atom, a halogen group, a cyano group, a nitro group, a trifluoromethyl group, a methyl group, an ethyl group, a propyl group, a butyl group, a pentyl group, a hexyl group, a phenyl group, a napthyl group, a biphenyl group, a triphenyl group, and a trifluoromethylphenyl group.

7. The compound as claimed in claim 1, wherein Z¹ and Z² are each independently selected from the group consisting of:

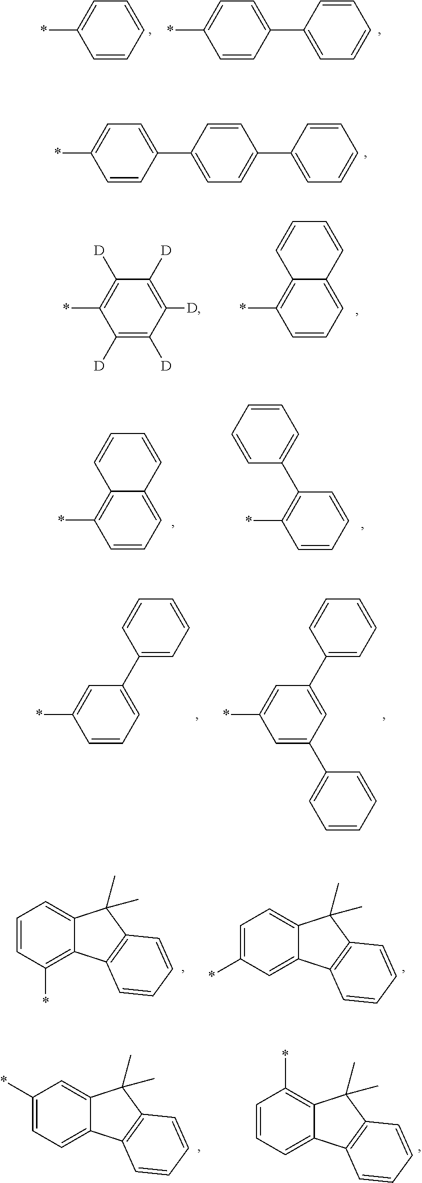

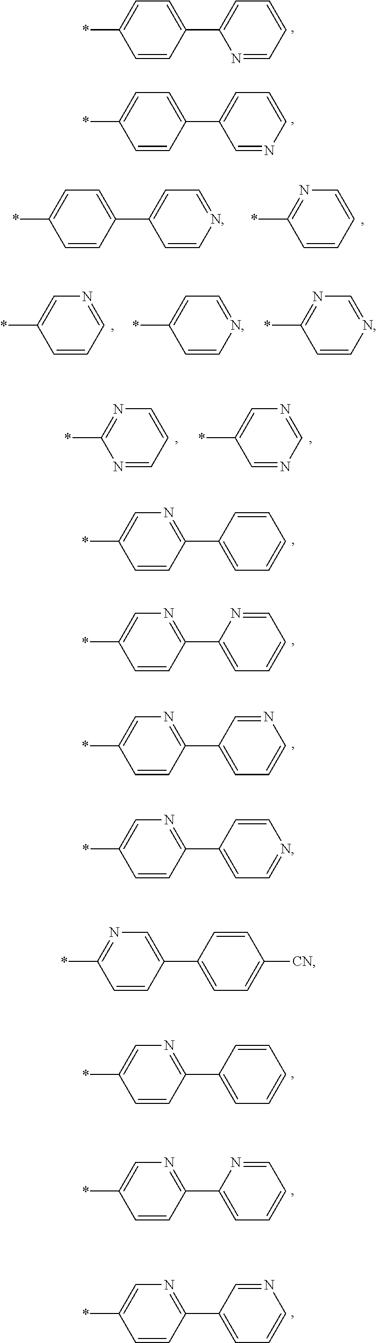

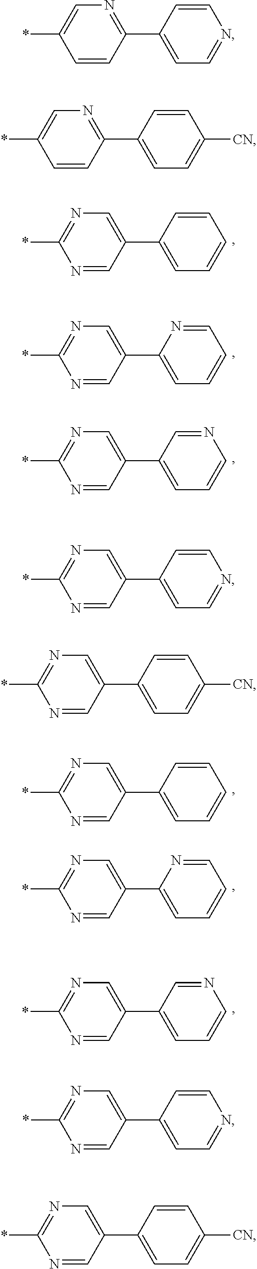

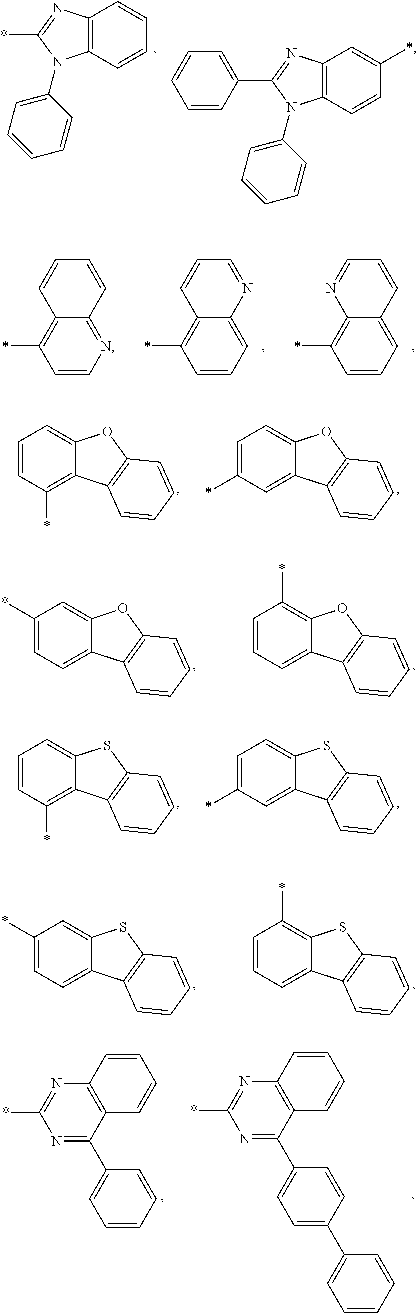

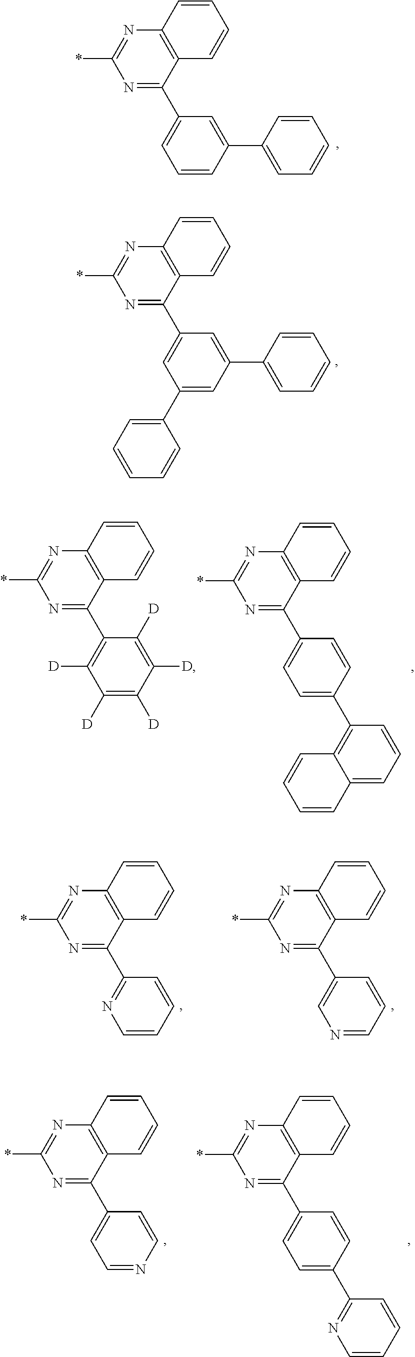

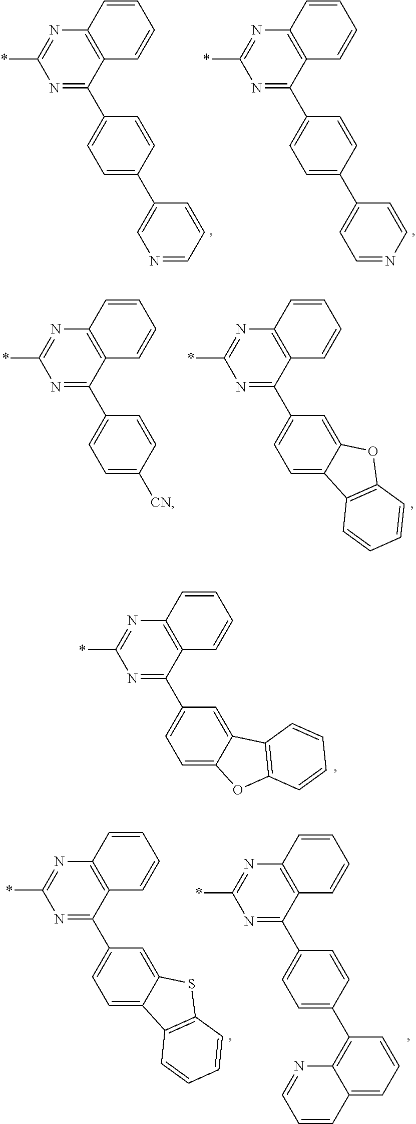

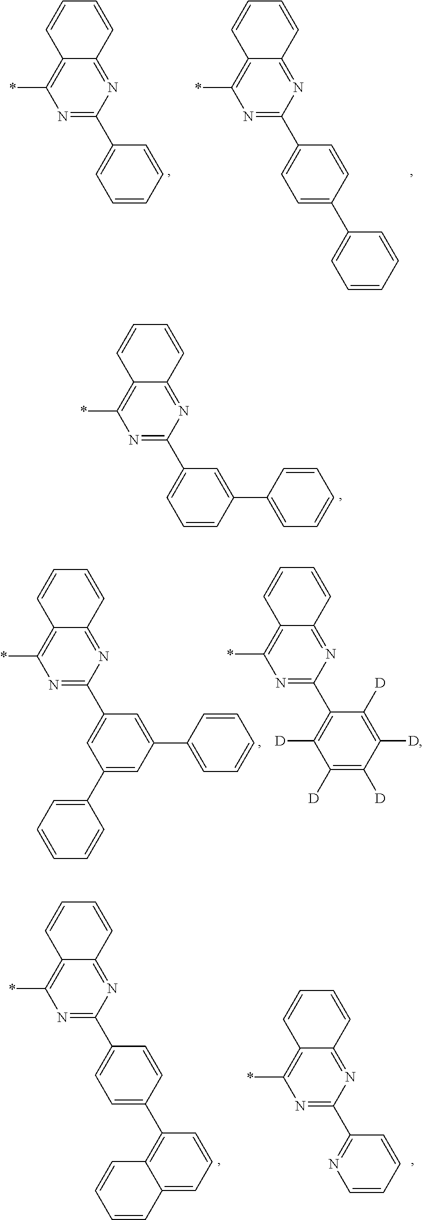

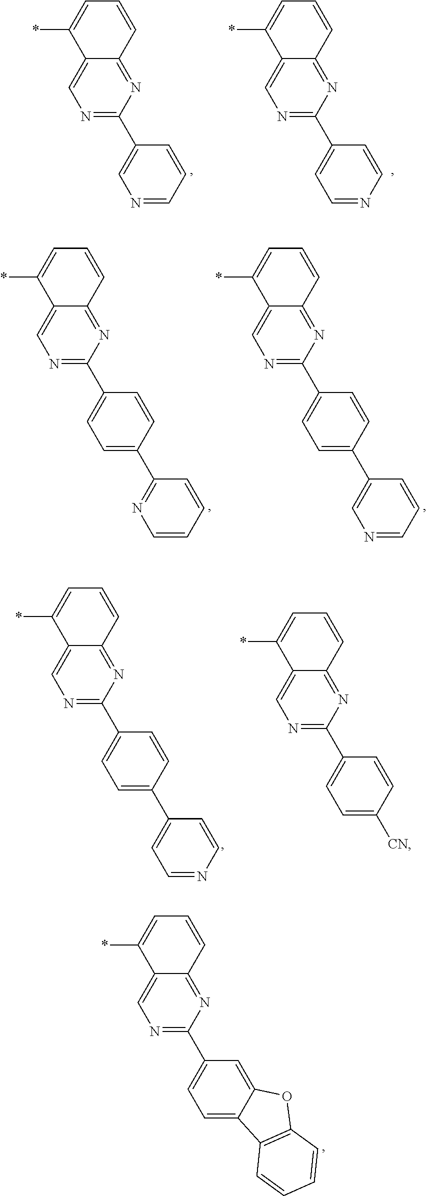

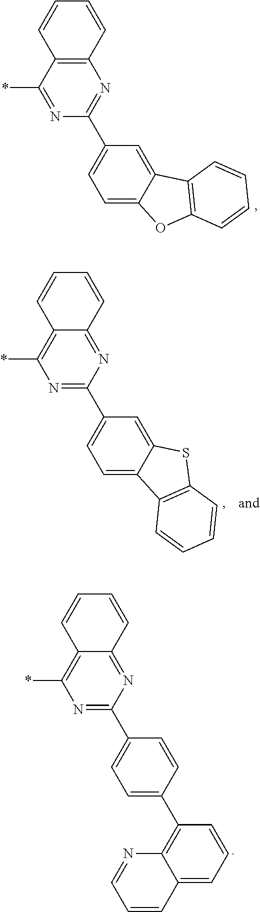 [SMILES: *c1nc(-c2ccc3oc4ccccc4c3c2)nc2ccccc12 ; *c1nc(-c2ccc3c(c2)sc2ccccc23)nc2ccccc12 ; *c1nc(-c2ccc(-c3cccc4cccnc34)cc2)nc2ccccc12]

8. The compound as claimed in claim 1, wherein the arylene group having 6 to 60 ring carbon atoms represented by L¹ to L⁴ are each independently selected from the group consisting of: 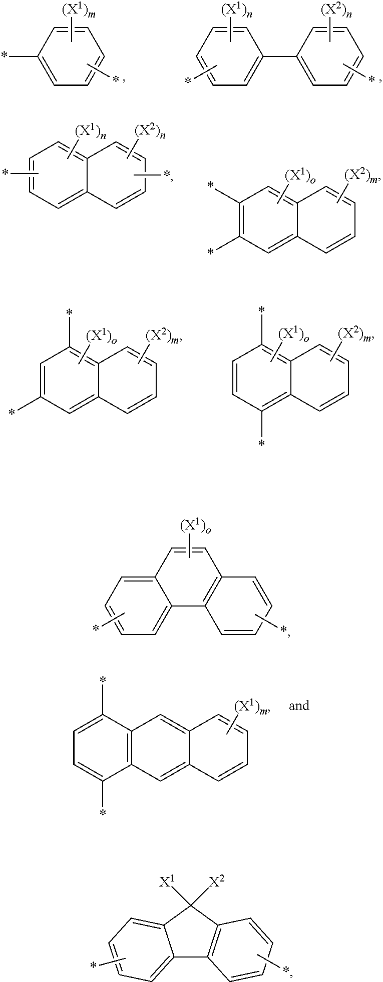
wherein m is an integer from 1 to 4, n is an integer from 1 to 3, and o is an integer 1 or 2;
X¹ to X² are each independently selected from the group consisting of: a hydrogen atom, a deuterium atom, a halo group, a cyano group, a nitro group, an alkyl group having 1 to 12 carbon atoms, an alkenyl group having 2 to 12 carbon atoms, an alkynyl group having 2 to 12 carbon atoms, an alkoxy group having 1 to 12 carbon atoms, an aryl group having 6 to 30 ring carbon atoms, a heteroaryl group having 3 to 30 ring carbon atoms, and an aryloxy group having 6 to 30 ring carbon atoms.

9. The compound as claimed in claim 1, wherein Y¹ to Y³ are each independently selected from the group consisting of: a hydrogen atom, a deuterium atom, a methyl group, an ethyl group, a propyl group, a butyl group, a pentyl group, a hexyl group, a phenyl group, a biphenyl group, and a napthyl group.

10. The compound as claimed in claim 1, wherein G² is selected from the group consisting of:

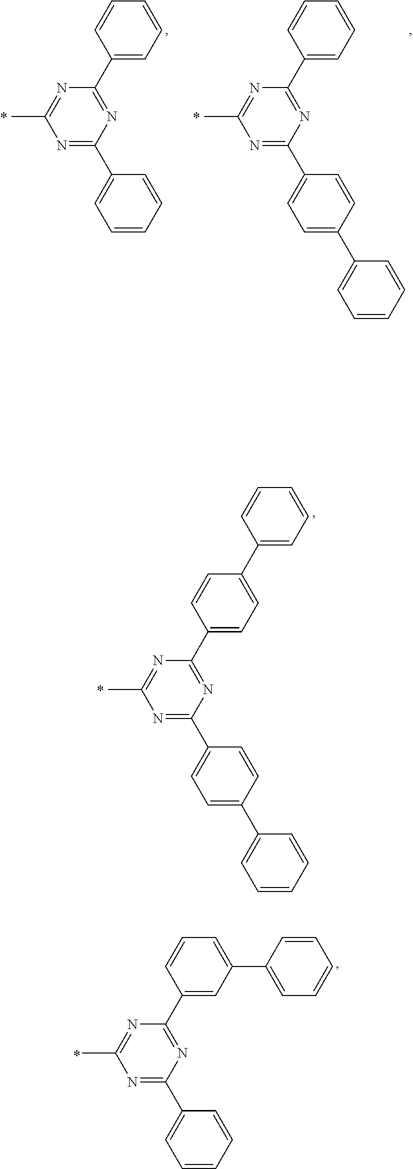 [SMILES: Cc1nc(-c2ccccc2)nc(-c2ccccc2)n1 ; Cc1nc(-c2ccccc2)nc(-c2ccc(-c3ccccc3)cc2)n1 ; Cc1nc(-c2ccc(-c3ccccc3)cc2)nc(-c2ccc(-c3ccccc3)cc2)n1 ; Cc1nc(-c2ccccc2)nc(-c2cccc(-c3ccccc3)c2)n1]

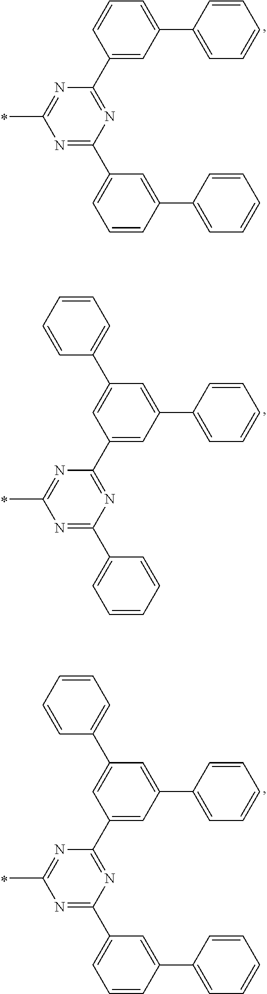 [SMILES: Cc1nc(-c2cccc(-c3ccccc3)c2)nc(-c2cccc(-c3ccccc3)c2)n1 ; Cc1nc(-c2ccccc2)nc(-c2cc(-c3ccccc3)cc(-c3ccccc3)c2)n1 ; Cc1nc(-c2cccc(-c3ccccc3)c2)nc(-c2cc(-c3ccccc3)cc(-c3ccccc3)c2)n1]

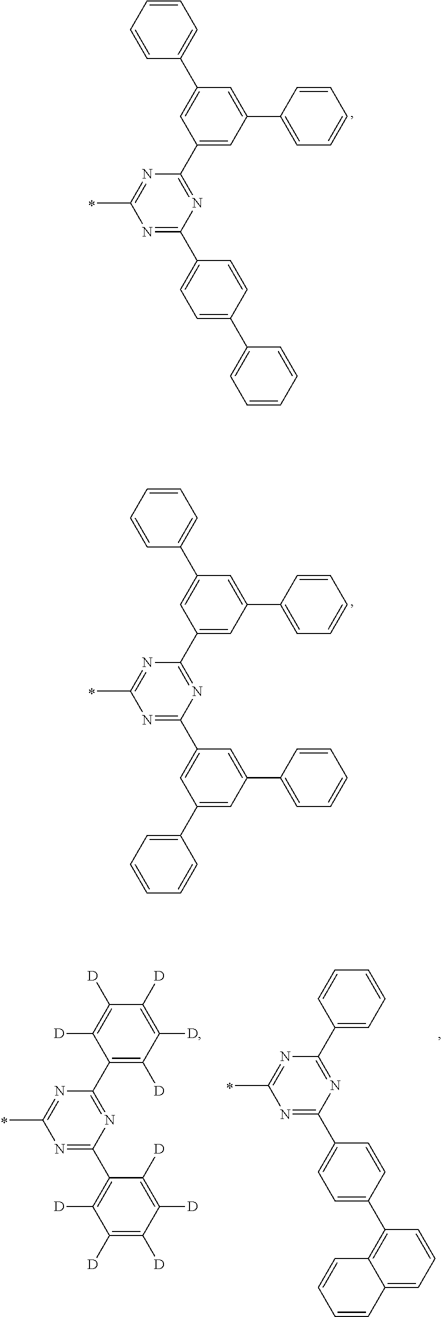

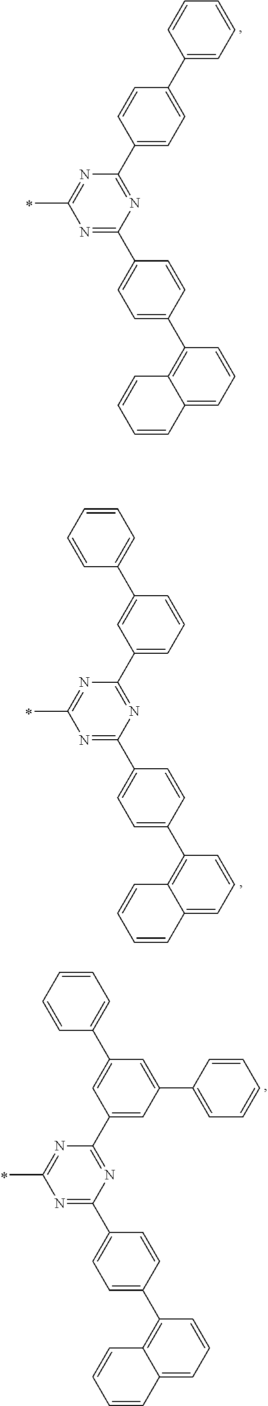 [SMILES: Cc1nc(-c2ccc(-c3ccccc3)cc2)nc(-c2ccc(-c3cccc4ccccc34)cc2)n1 ; Cc1nc(-c2ccc(-c3cccc4ccccc34)cc2)nc(-c2cccc(-c3ccccc3)c2)n1 ; Cc1nc(-c2ccc(-c3cccc4ccccc34)cc2)nc(-c2cc(-c3ccccc3)cc(-c3ccccc3)c2)n1]

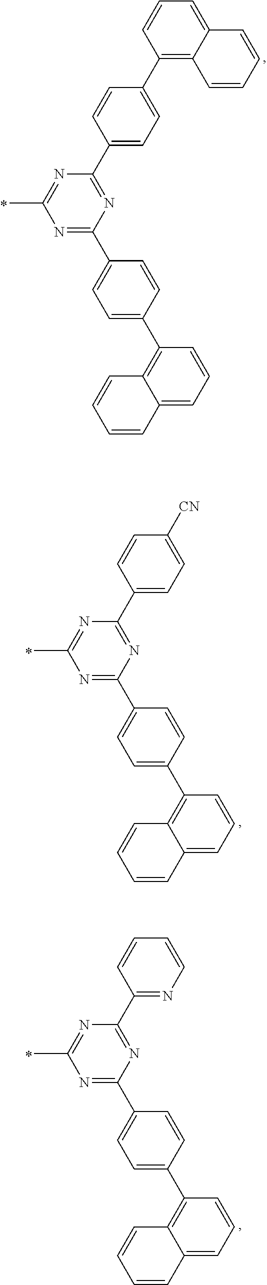 [SMILES: Cc1nc(-c2ccc(-c3cccc4ccccc34)cc2)nc(-c2ccc(-c3cccc4ccccc34)cc2)n1 ; Cc1nc(-c2ccc(C#N)cc2)nc(-c2ccc(-c3cccc4ccccc34)cc2)n1 ; Cc1nc(-c2ccc(-c3cccc4ccccc34)cc2)nc(-c2ccccn2)n1]

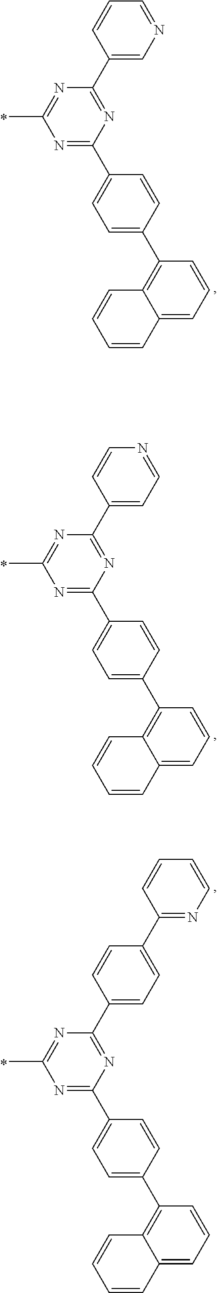 [SMILES: Cc1nc(-c2ccc(-c3cccc4ccccc34)cc2)nc(-c2cccnc2)n1 ; Cc1nc(-c2ccncc2)nc(-c2ccc(-c3cccc4ccccc34)cc2)n1 ; Cc1nc(-c2ccc(-c3ccccn3)cc2)nc(-c2ccc(-c3cccc4ccccc34)cc2)n1]

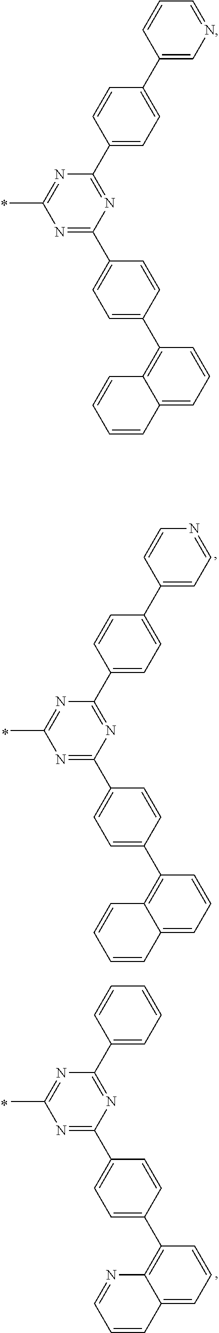

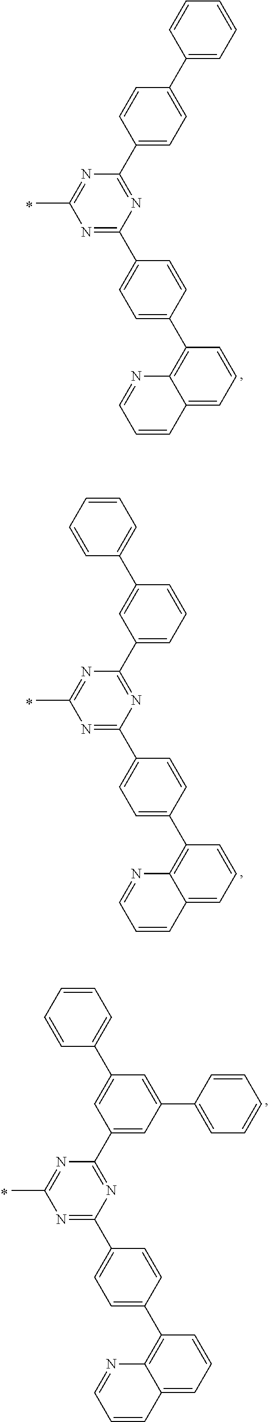 [SMILES: Cc1nc(-c2ccc(-c3ccccc3)cc2)nc(-c2ccc(-c3cccc4cccnc34)cc2)n1 ; Cc1nc(-c2ccc(-c3cccc4cccnc34)cc2)nc(-c2cccc(-c3ccccc3)c2)n1 ; Cc1nc(-c2ccc(-c3cccc4cccnc34)cc2)nc(-c2cc(-c3ccccc3)cc(-c3ccccc3)c2)n1]

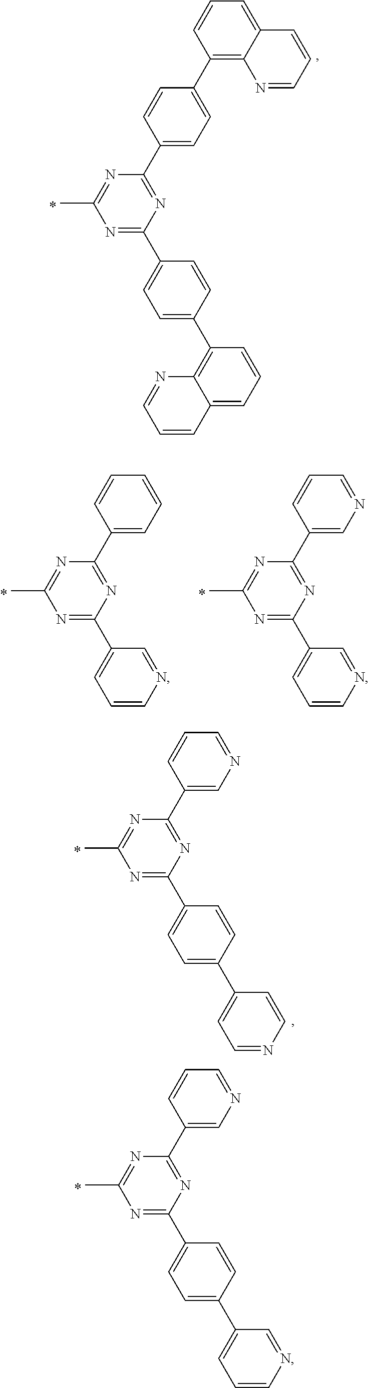

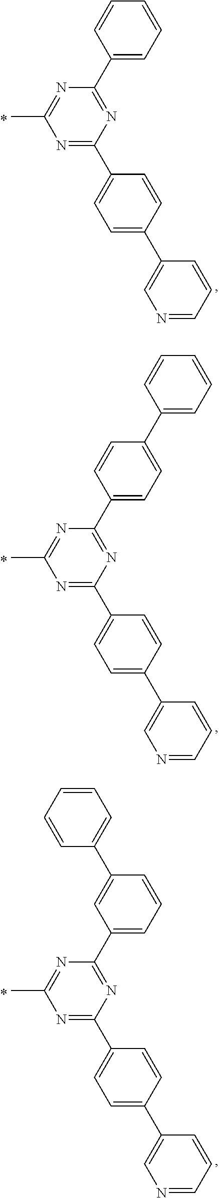

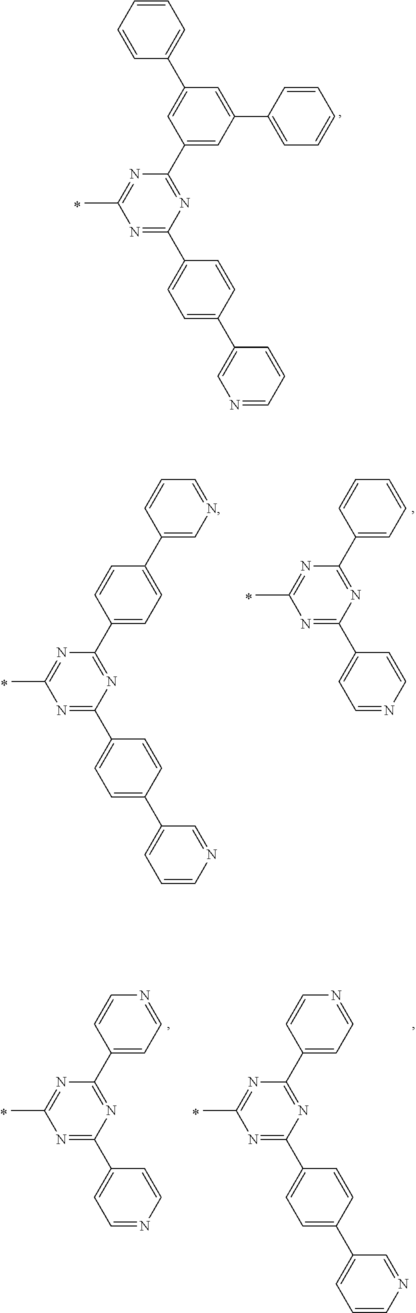 [SMILES: Cc1nc(-c2ccc(-c3cccnc3)cc2)nc(-c2cc(-c3ccccc3)cc(-c3ccccc3)c2)n1 ; Cc1nc(-c2ccc(-c3cccnc3)cc2)nc(-c2ccc(-c3cccnc3)cc2)n1 ; Cc1nc(-c2ccccc2)nc(-c2ccncc2)n1 ; Cc1nc(-c2ccncc2)nc(-c2ccncc2)n1 ; Cc1nc(-c2ccncc2)nc(-c2ccc(-c3cccnc3)cc2)n1]

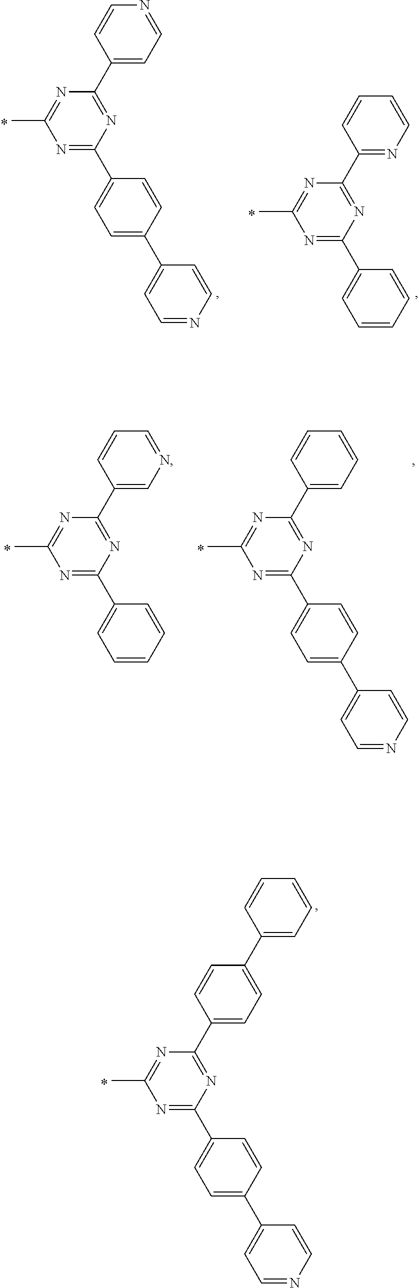 [SMILES: Cc1nc(-c2ccncc2)nc(-c2ccc(-c3ccncc3)cc2)n1 ; Cc1nc(-c2ccccc2)nc(-c2ccccn2)n1 ; Cc1nc(-c2ccccc2)nc(-c2cccnc2)n1 ; Cc1nc(-c2ccccc2)nc(-c2ccc(-c3ccncc3)cc2)n1 ; Cc1nc(-c2ccc(-c3ccccc3)cc2)nc(-c2ccc(-c3ccncc3)cc2)n1]

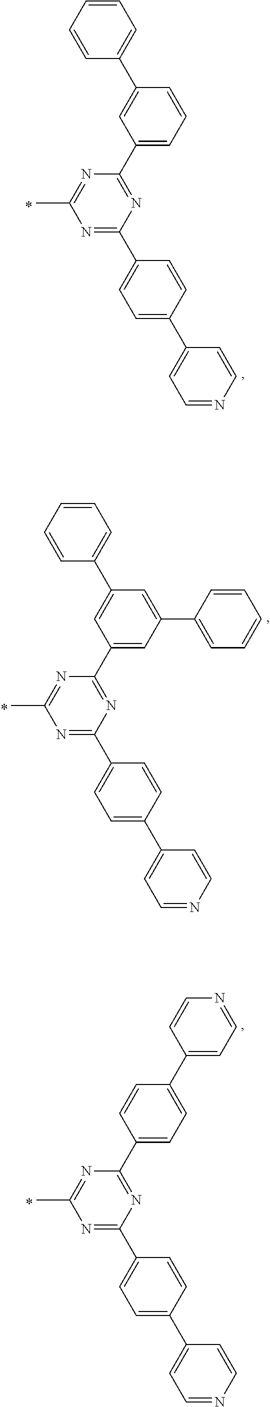 [SMILES: Cc1nc(-c2ccc(-c3ccncc3)cc2)nc(-c2cccc(-c3ccccc3)c2)n1 ; Cc1nc(-c2ccc(-c3ccncc3)cc2)nc(-c2cc(-c3ccccc3)cc(-c3ccccc3)c2)n1 ; Cc1nc(-c2ccc(-c3ccncc3)cc2)nc(-c2ccc(-c3ccncc3)cc2)n1]

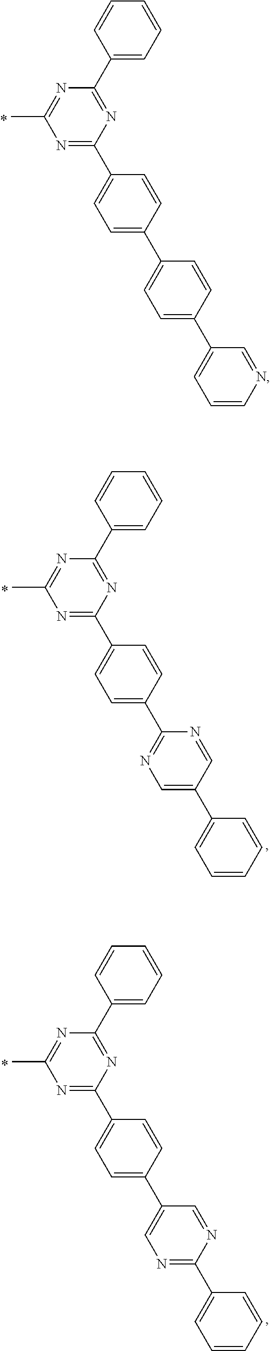 [SMILES: Cc1nc(-c2ccccc2)nc(-c2ccc(-c3ccc(-c4cccnc4)cc3)cc2)n1 ; Cc1nc(-c2ccccc2)nc(-c2ccc(-c3ncc(-c4ccccc4)cn3)cc2)n1 ; Cc1nc(-c2ccccc2)nc(-c2ccc(-c3cnc(-c4ccccc4)nc3)cc2)n1]

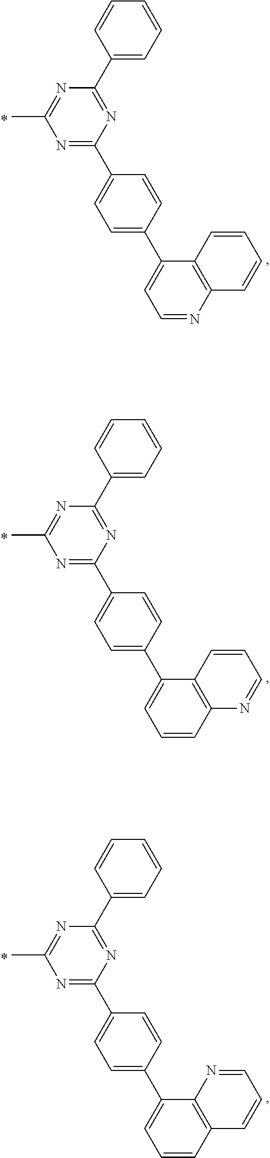 [SMILES: Cc1nc(-c2ccccc2)nc(-c2ccc(-c3ccnc4ccccc34)cc2)n1 ; Cc1nc(-c2ccccc2)nc(-c2ccc(-c3cccc4ncccc34)cc2)n1 ; Cc1nc(-c2ccccc2)nc(-c2ccc(-c3cccc4cccnc34)cc2)n1]

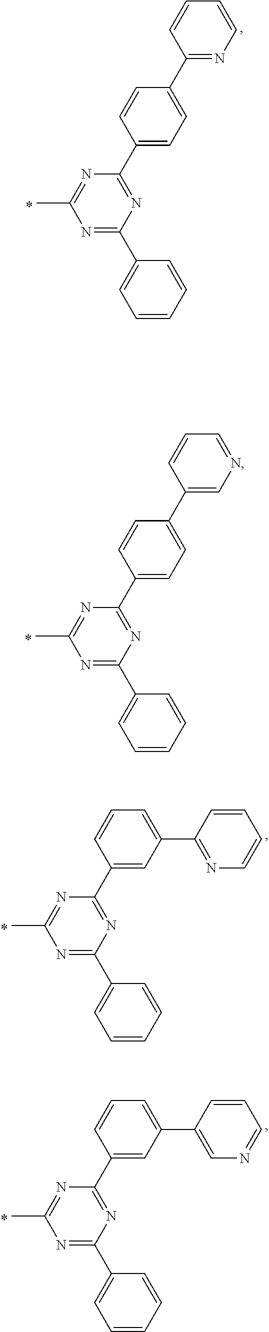 [SMILES: Cc1nc(-c2ccccc2)nc(-c2ccc(-c3ccccn3)cc2)n1 ; Cc1nc(-c2ccccc2)nc(-c2ccc(-c3cccnc3)cc2)n1 ; Cc1nc(-c2ccccc2)nc(-c2cccc(-c3ccccn3)c2)n1 ; Cc1nc(-c2ccccc2)nc(-c2cccc(-c3cccnc3)c2)n1]

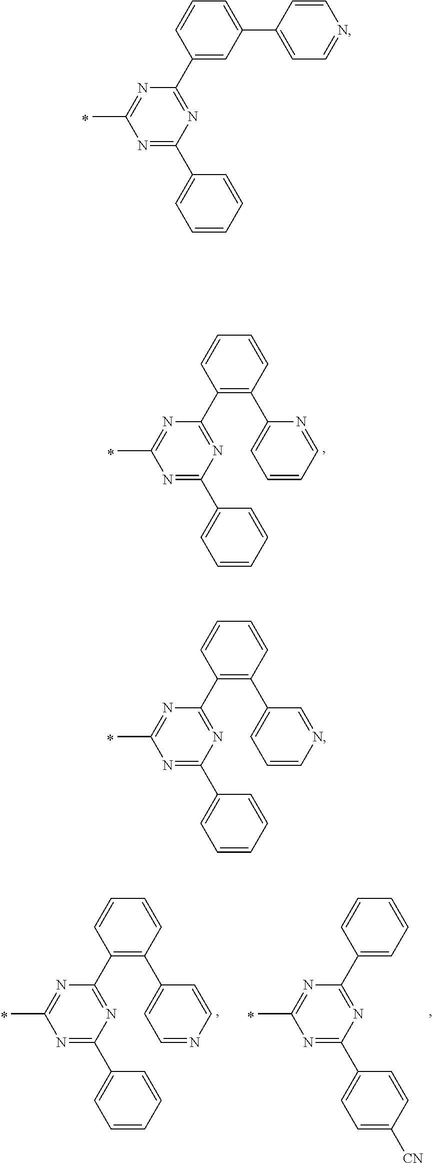 [SMILES: Cc1nc(-c2ccccc2)nc(-c2cccc(-c3ccncc3)c2)n1 ; Cc1nc(-c2ccccc2)nc(-c2ccccc2-c2ccccn2)n1 ; Cc1nc(-c2ccccc2)nc(-c2ccccc2-c2cccnc2)n1 ; Cc1nc(-c2ccccc2)nc(-c2ccccc2-c2ccncc2)n1 ; Cc1nc(-c2ccccc2)nc(-c2ccc(C#N)cc2)n1]

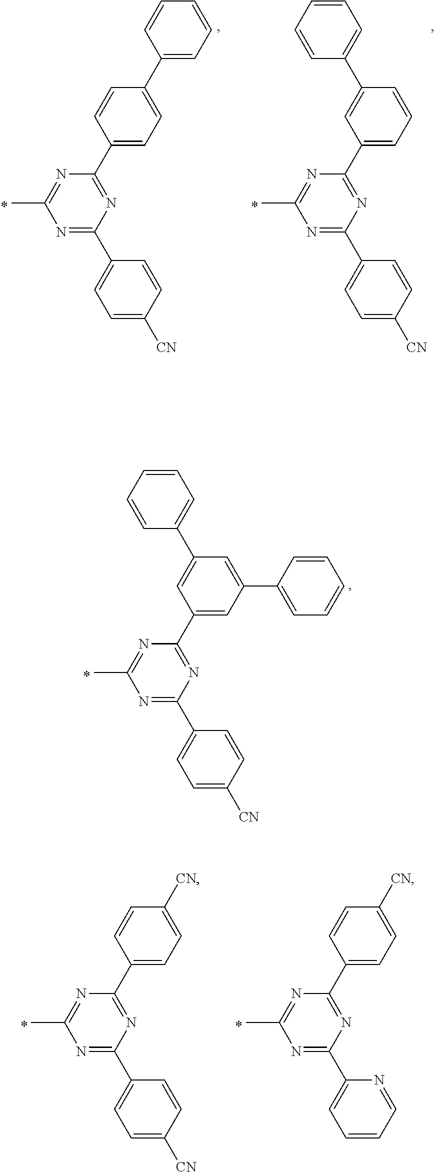 [SMILES: Cc1nc(-c2ccc(C#N)cc2)nc(-c2ccc(-c3ccccc3)cc2)n1 ; Cc1nc(-c2ccc(C#N)cc2)nc(-c2cccc(-c3ccccc3)c2)n1 ; Cc1nc(-c2ccc(C#N)cc2)nc(-c2cc(-c3ccccc3)cc(-c3ccccc3)c2)n1 ; Cc1nc(-c2ccc(C#N)cc2)nc(-c2ccc(C#N)cc2)n1 ; Cc1nc(-c2ccc(C#N)cc2)nc(-c2ccccn2)n1]

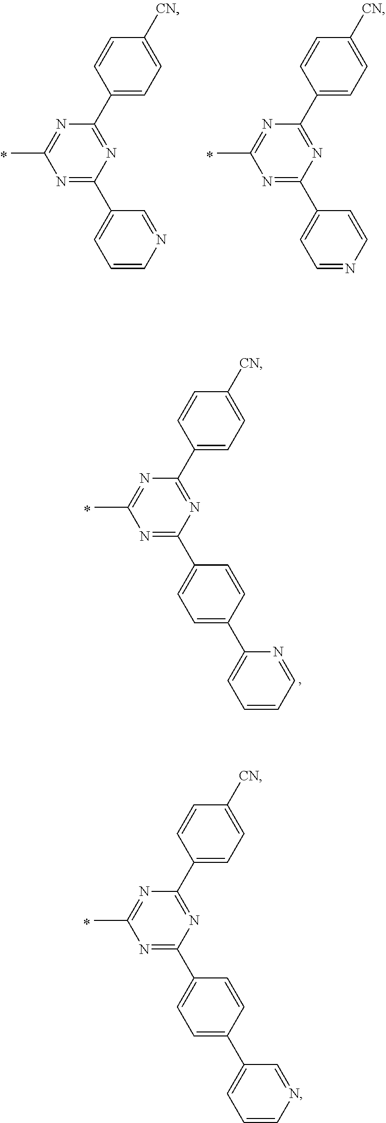 [SMILES: Cc1nc(-c2ccc(C#N)cc2)nc(-c2cccnc2)n1 ; Cc1nc(-c2ccncc2)nc(-c2ccc(C#N)cc2)n1 ; Cc1nc(-c2ccc(C#N)cc2)nc(-c2ccc(-c3ccccn3)cc2)n1 ; Cc1nc(-c2ccc(C#N)cc2)nc(-c2ccc(-c3cccnc3)cc2)n1]

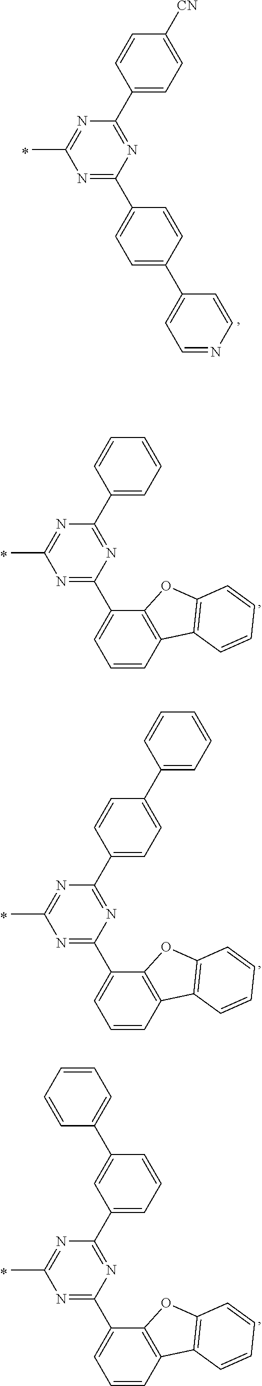

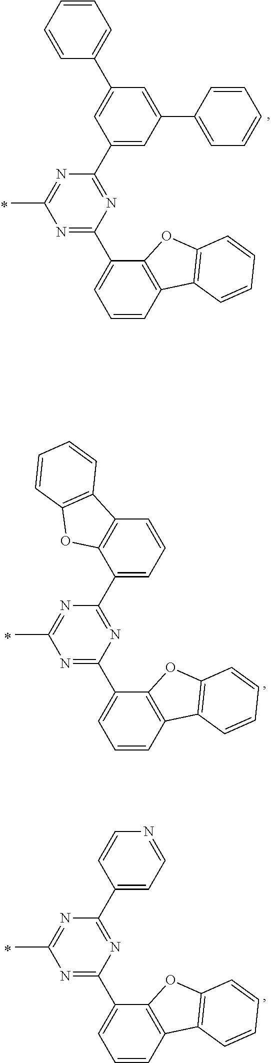 [SMILES: Cc1nc(-c2cc(-c3ccccc3)cc(-c3ccccc3)c2)nc(-c2cccc3c2oc2ccccc23)n1 ; Cc1nc(-c2cccc3c2oc2ccccc23)nc(-c2cccc3c2oc2ccccc23)n1 ; Cc1nc(-c2ccncc2)nc(-c2cccc3c2oc2ccccc23)n1]

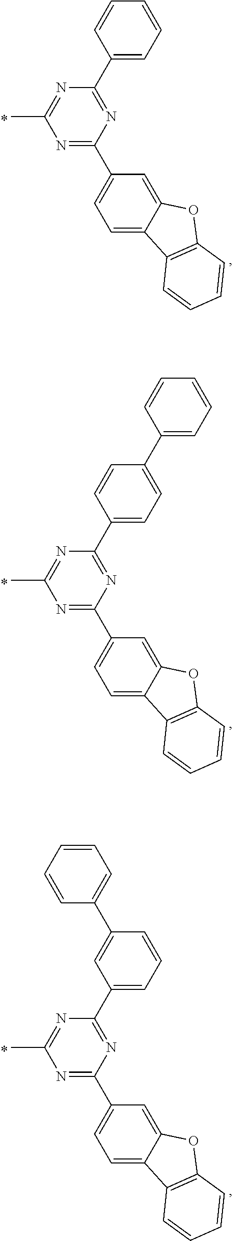 [SMILES: Cc1nc(-c2ccccc2)nc(-c2ccc3c(c2)oc2ccccc23)n1 ; Cc1nc(-c2ccc(-c3ccccc3)cc2)nc(-c2ccc3c(c2)oc2ccccc23)n1 ; Cc1nc(-c2cccc(-c3ccccc3)c2)nc(-c2ccc3c(c2)oc2ccccc23)n1]

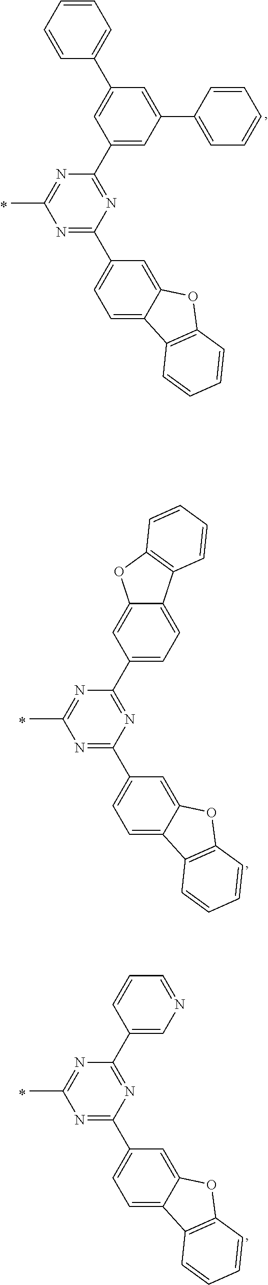 [SMILES: Cc1nc(-c2cc(-c3ccccc3)cc(-c3ccccc3)c2)nc(-c2ccc3c(c2)oc2ccccc23)n1 ; Cc1nc(-c2ccc3c(c2)oc2ccccc23)nc(-c2ccc3c(c2)oc2ccccc23)n1 ; Cc1nc(-c2cccnc2)nc(-c2ccc3c(c2)oc2ccccc23)n1]

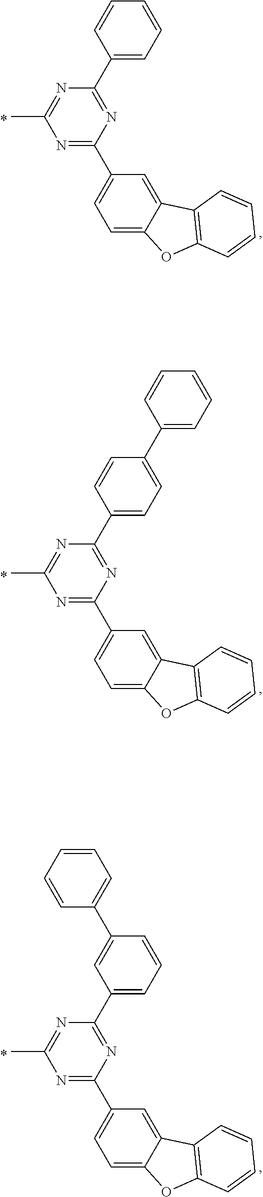 [SMILES: Cc1nc(-c2ccccc2)nc(-c2ccc3oc4ccccc4c3c2)n1 ; Cc1nc(-c2ccc(-c3ccccc3)cc2)nc(-c2ccc3oc4ccccc4c3c2)n1 ; Cc1nc(-c2cccc(-c3ccccc3)c2)nc(-c2ccc3oc4ccccc4c3c2)n1]

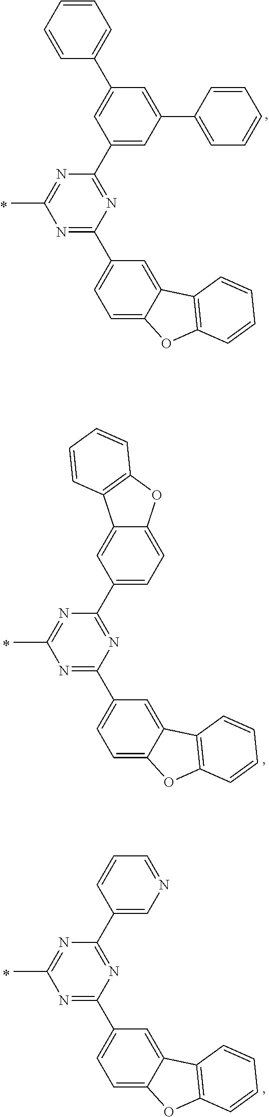 [SMILES: Cc1nc(-c2cc(-c3ccccc3)cc(-c3ccccc3)c2)nc(-c2ccc3oc4ccccc4c3c2)n1 ; Cc1nc(-c2ccc3oc4ccccc4c3c2)nc(-c2ccc3oc4ccccc4c3c2)n1 ; Cc1nc(-c2cccnc2)nc(-c2ccc3oc4ccccc4c3c2)n1]

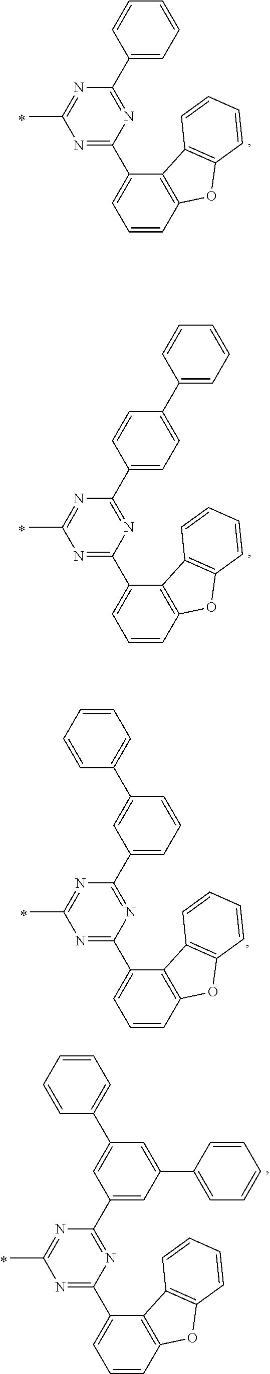

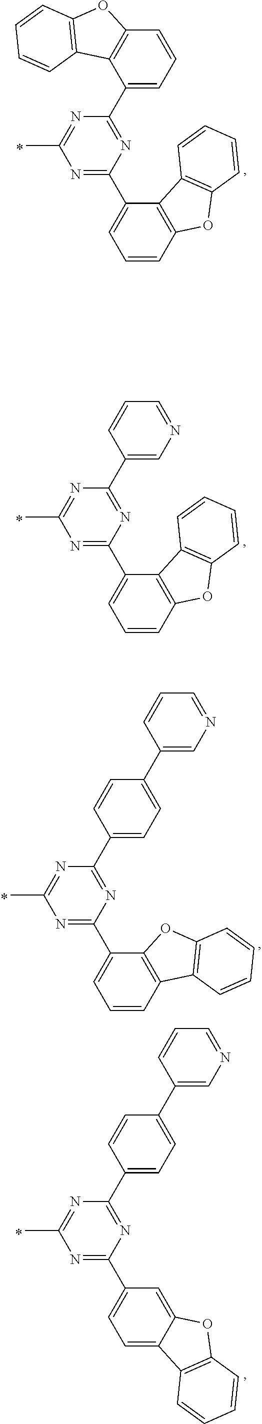

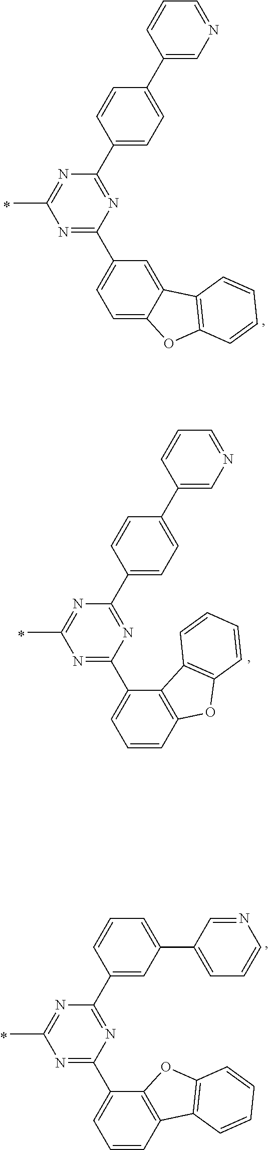 [SMILES: Cc1nc(-c2ccc(-c3cccnc3)cc2)nc(-c2ccc3oc4ccccc4c3c2)n1 ; Cc1nc(-c2ccc(-c3cccnc3)cc2)nc(-c2cccc3oc4ccccc4c23)n1 ; Cc1nc(-c2cccc(-c3cccnc3)c2)nc(-c2cccc3c2oc2ccccc23)n1]

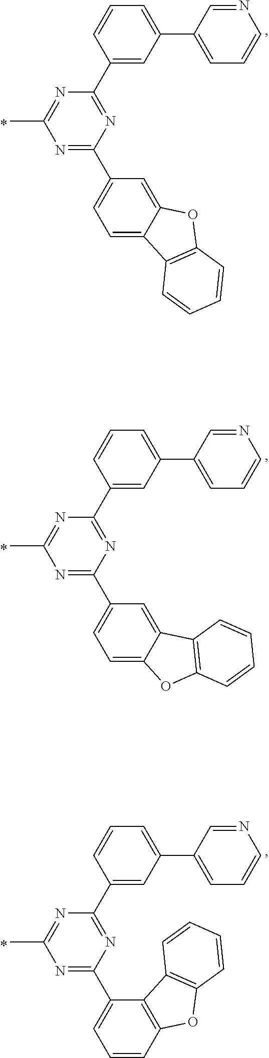 [SMILES: Cc1nc(-c2cccc(-c3cccnc3)c2)nc(-c2ccc3c(c2)oc2ccccc23)n1 ; Cc1nc(-c2cccc(-c3cccnc3)c2)nc(-c2ccc3oc4ccccc4c3c2)n1 ; Cc1nc(-c2cccc(-c3cccnc3)c2)nc(-c2cccc3oc4ccccc4c23)n1]

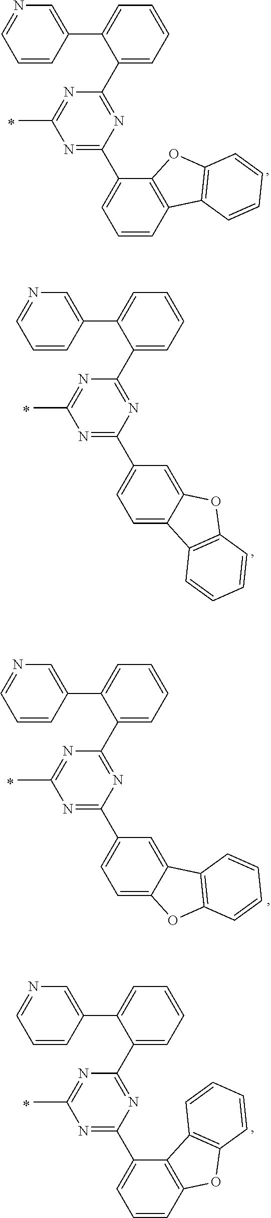 [SMILES: Cc1nc(-c2ccccc2-c2cccnc2)nc(-c2cccc3c2oc2ccccc23)n1 ; Cc1nc(-c2ccc3c(c2)oc2ccccc23)nc(-c2ccccc2-c2cccnc2)n1 ; Cc1nc(-c2ccc3oc4ccccc4c3c2)nc(-c2ccccc2-c2cccnc2)n1 ; Cc1nc(-c2ccccc2-c2cccnc2)nc(-c2cccc3oc4ccccc4c23)n1]

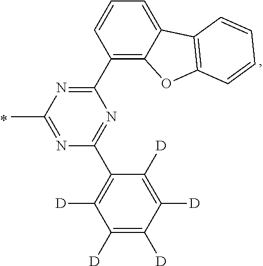 [SMILES: [2H]c1c([2H])c([2H])c(-c2nc(C)nc(-c3cccc4c3oc3ccccc34)n2)c([2H])c1[2H]]

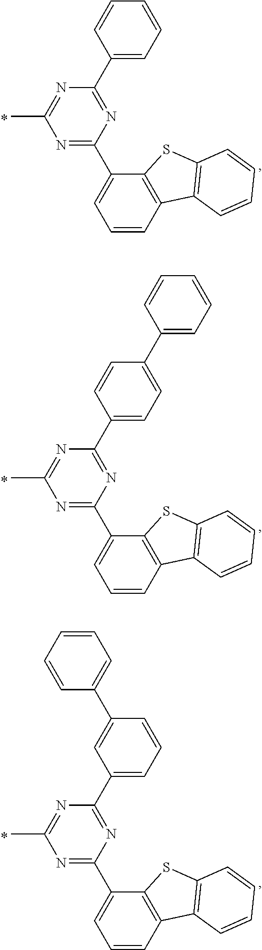

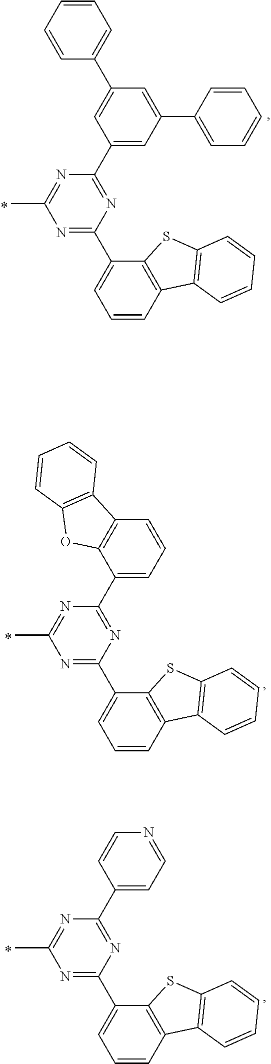 [SMILES: Cc1nc(-c2cc(-c3ccccc3)cc(-c3ccccc3)c2)nc(-c2cccc3c2sc2ccccc23)n1 ; Cc1nc(-c2cccc3c2oc2ccccc23)nc(-c2cccc3c2sc2ccccc23)n1 ; Cc1nc(-c2ccncc2)nc(-c2cccc3c2sc2ccccc23)n1]

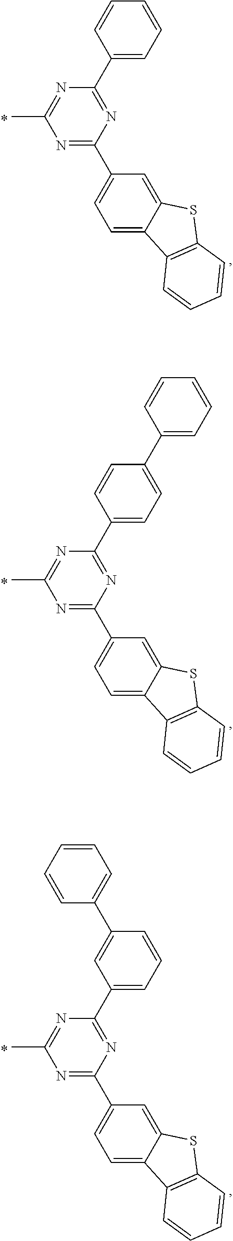 [SMILES: Cc1nc(-c2ccccc2)nc(-c2ccc3c(c2)sc2ccccc23)n1 ; Cc1nc(-c2ccc(-c3ccccc3)cc2)nc(-c2ccc3c(c2)sc2ccccc23)n1 ; Cc1nc(-c2cccc(-c3ccccc3)c2)nc(-c2ccc3c(c2)sc2ccccc23)n1]

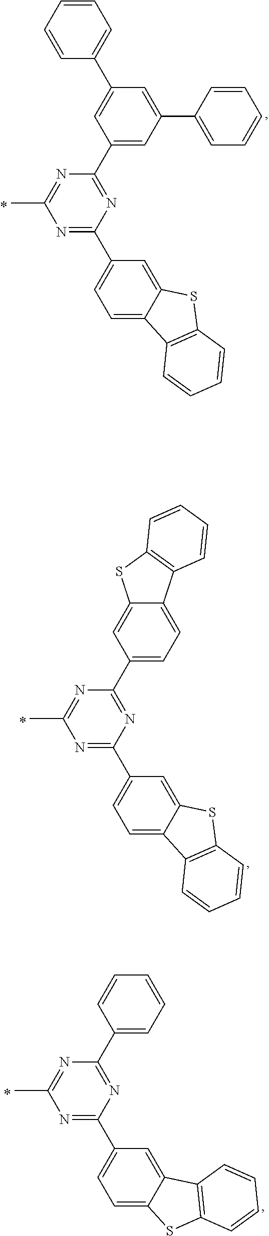 [SMILES: Cc1nc(-c2cc(-c3ccccc3)cc(-c3ccccc3)c2)nc(-c2ccc3c(c2)sc2ccccc23)n1 ; Cc1nc(-c2ccc3c(c2)sc2ccccc23)nc(-c2ccc3c(c2)sc2ccccc23)n1 ; Cc1nc(-c2ccccc2)nc(-c2ccc3sc4ccccc4c3c2)n1]

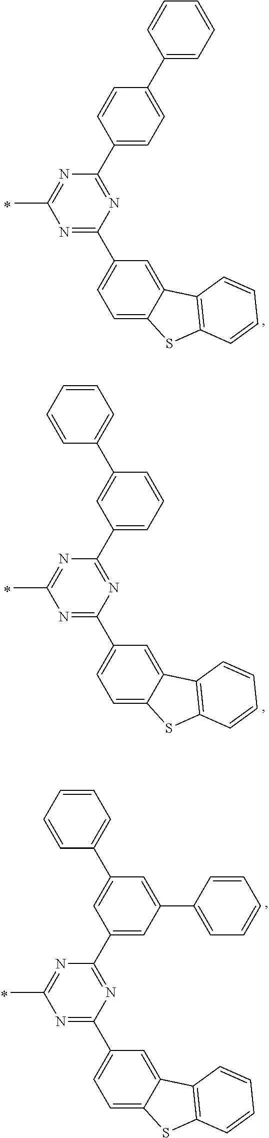

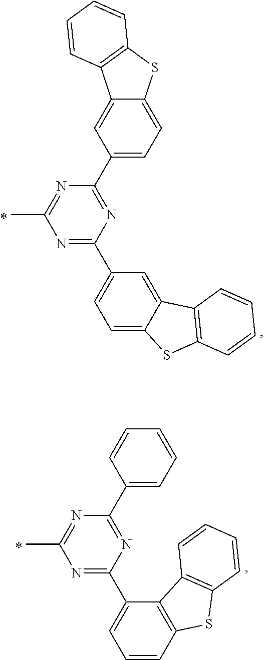 [SMILES: Cc1nc(-c2ccc3sc4ccccc4c3c2)nc(-c2ccc3sc4ccccc4c3c2)n1 ; Cc1nc(-c2ccccc2)nc(-c2cccc3sc4ccccc4c23)n1]

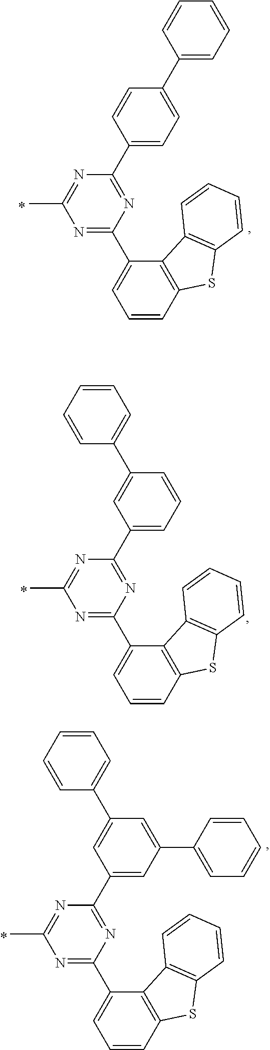

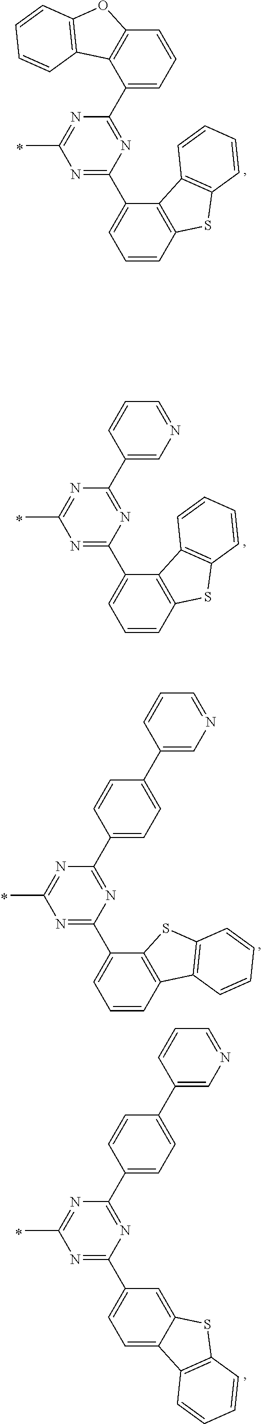

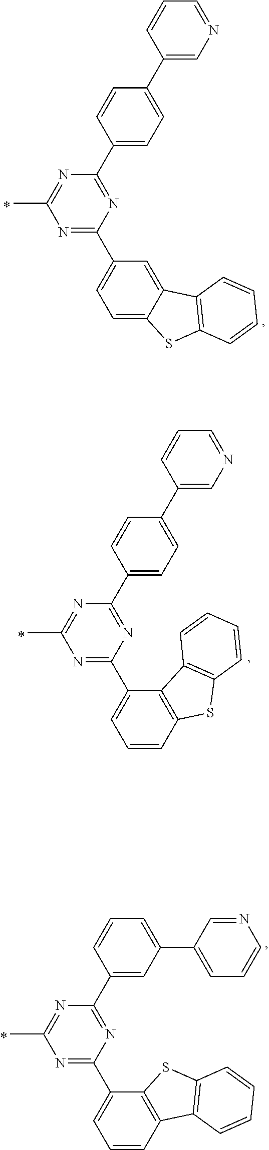 [SMILES: Cc1nc(-c2ccc(-c3cccnc3)cc2)nc(-c2ccc3sc4ccccc4c3c2)n1 ; Cc1nc(-c2ccc(-c3cccnc3)cc2)nc(-c2cccc3sc4ccccc4c23)n1 ; Cc1nc(-c2cccc(-c3cccnc3)c2)nc(-c2cccc3c2sc2ccccc23)n1]

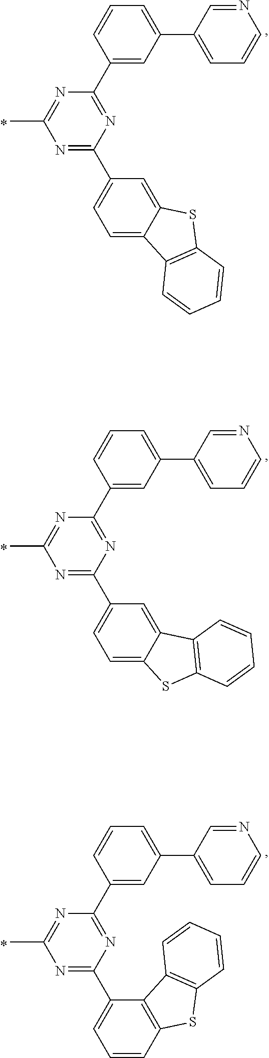 [SMILES: Cc1nc(-c2cccc(-c3cccnc3)c2)nc(-c2ccc3c(c2)sc2ccccc23)n1 ; Cc1nc(-c2cccc(-c3cccnc3)c2)nc(-c2ccc3sc4ccccc4c3c2)n1 ; Cc1nc(-c2cccc(-c3cccnc3)c2)nc(-c2cccc3sc4ccccc4c23)n1]

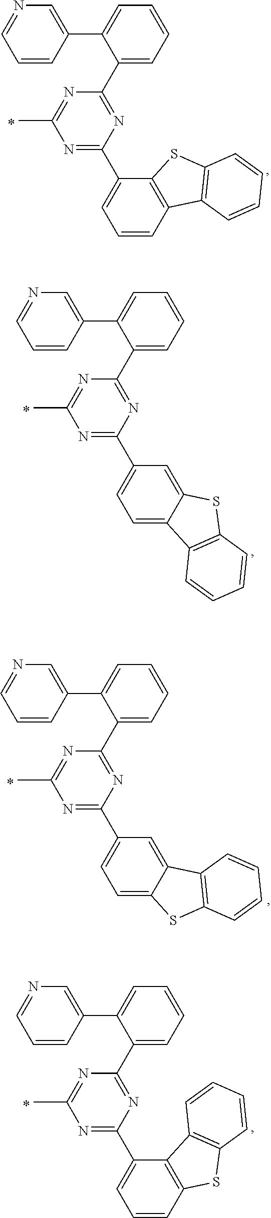 [SMILES: Cc1nc(-c2ccccc2-c2cccnc2)nc(-c2cccc3c2sc2ccccc23)n1 ; Cc1nc(-c2ccc3c(c2)sc2ccccc23)nc(-c2ccccc2-c2cccnc2)n1 ; Cc1nc(-c2ccc3sc4ccccc4c3c2)nc(-c2ccccc2-c2cccnc2)n1 ; Cc1nc(-c2ccccc2-c2cccnc2)nc(-c2cccc3sc4ccccc4c23)n1]

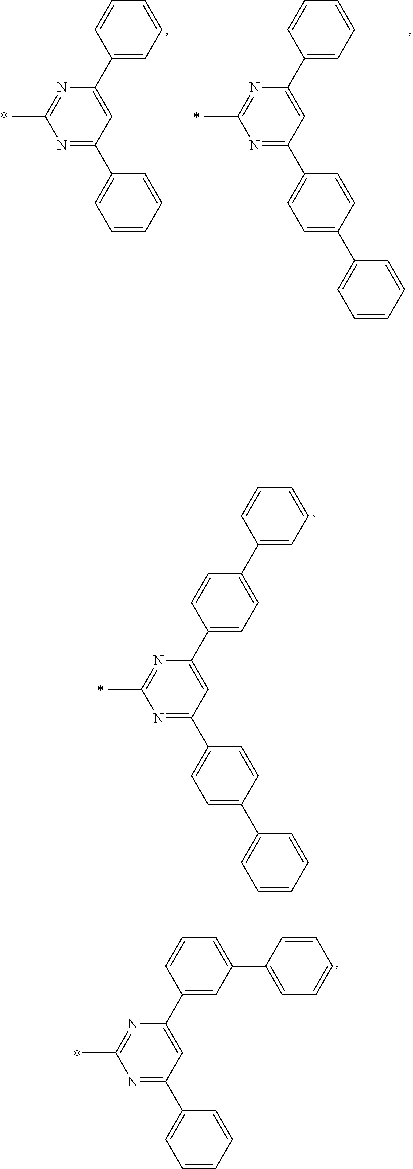 [SMILES: Cc1nc(-c2ccccc2)cc(-c2ccccc2)n1 ; Cc1nc(-c2ccccc2)cc(-c2ccc(-c3ccccc3)cc2)n1 ; Cc1nc(-c2ccc(-c3ccccc3)cc2)cc(-c2ccc(-c3ccccc3)cc2)n1 ; Cc1nc(-c2ccccc2)cc(-c2cccc(-c3ccccc3)c2)n1]

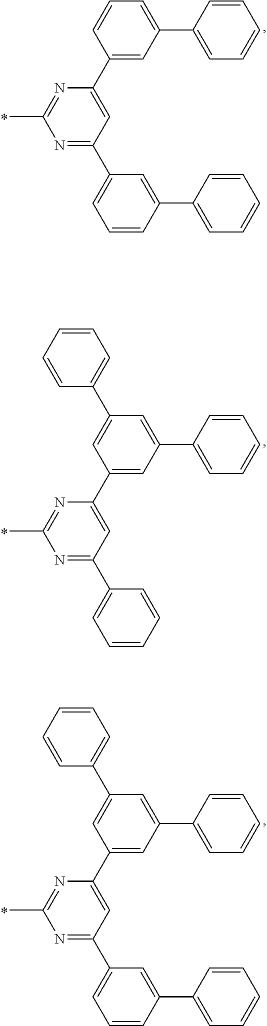 [SMILES: Cc1nc(-c2cccc(-c3ccccc3)c2)cc(-c2cccc(-c3ccccc3)c2)n1 ; Cc1nc(-c2ccccc2)cc(-c2cc(-c3ccccc3)cc(-c3ccccc3)c2)n1 ; Cc1nc(-c2cccc(-c3ccccc3)c2)cc(-c2cc(-c3ccccc3)cc(-c3ccccc3)c2)n1]

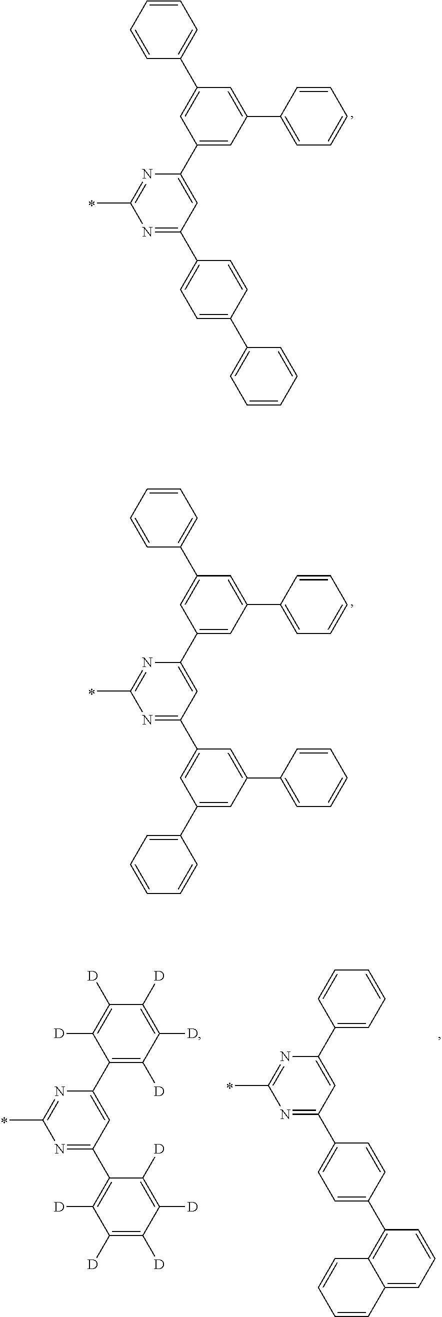

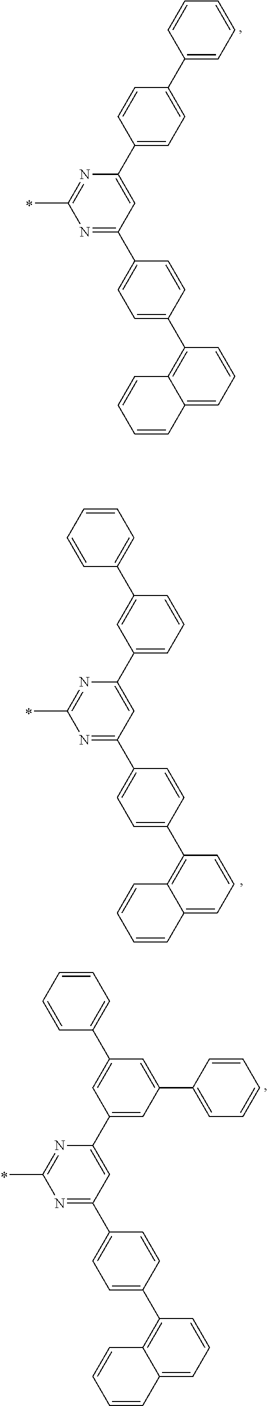 [SMILES: Cc1nc(-c2ccc(-c3ccccc3)cc2)cc(-c2ccc(-c3cccc4ccccc34)cc2)n1 ; Cc1nc(-c2ccc(-c3cccc4ccccc34)cc2)cc(-c2cccc(-c3ccccc3)c2)n1 ; Cc1nc(-c2ccc(-c3cccc4ccccc34)cc2)cc(-c2cc(-c3ccccc3)cc(-c3ccccc3)c2)n1]

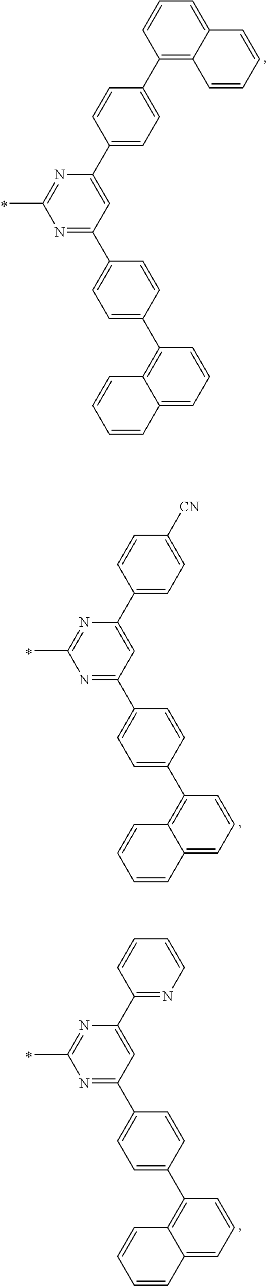 [SMILES: Cc1nc(-c2ccc(-c3cccc4ccccc34)cc2)cc(-c2ccc(-c3cccc4ccccc34)cc2)n1 ; Cc1nc(-c2ccc(C#N)cc2)cc(-c2ccc(-c3cccc4ccccc34)cc2)n1 ; Cc1nc(-c2ccc(-c3cccc4ccccc34)cc2)cc(-c2ccccn2)n1]

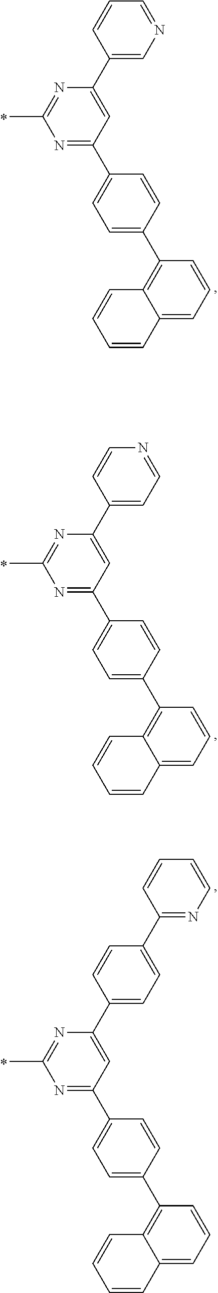 [SMILES: Cc1nc(-c2ccc(-c3cccc4ccccc34)cc2)cc(-c2cccnc2)n1 ; Cc1nc(-c2ccncc2)cc(-c2ccc(-c3cccc4ccccc34)cc2)n1 ; Cc1nc(-c2ccc(-c3ccccn3)cc2)cc(-c2ccc(-c3cccc4ccccc34)cc2)n1]

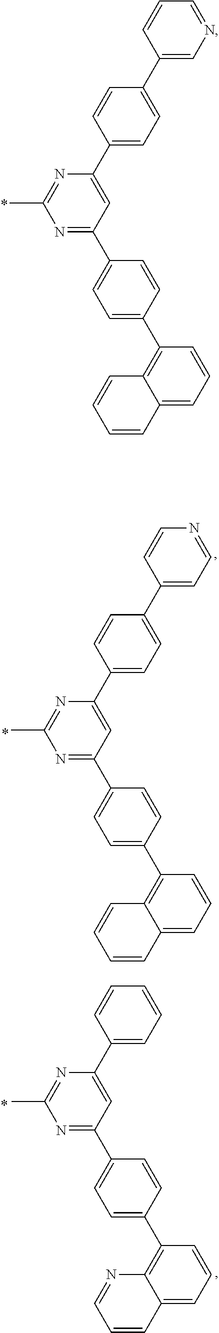

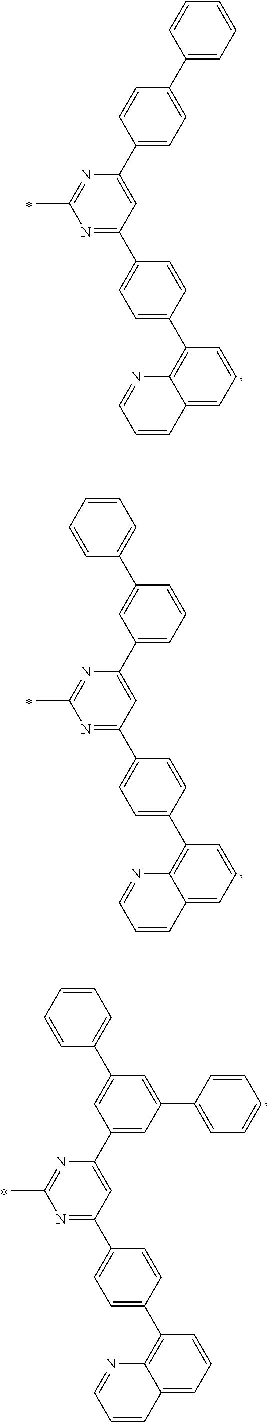 [SMILES: Cc1nc(-c2ccc(-c3ccccc3)cc2)cc(-c2ccc(-c3cccc4cccnc34)cc2)n1 ; Cc1nc(-c2ccc(-c3cccc4cccnc34)cc2)cc(-c2cccc(-c3ccccc3)c2)n1 ; Cc1nc(-c2ccc(-c3cccc4cccnc34)cc2)cc(-c2cc(-c3ccccc3)cc(-c3ccccc3)c2)n1]

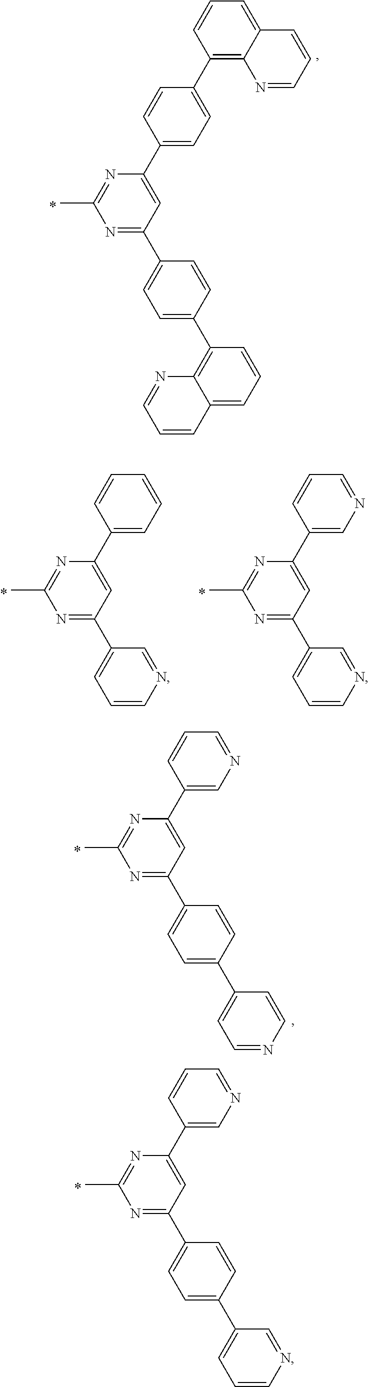

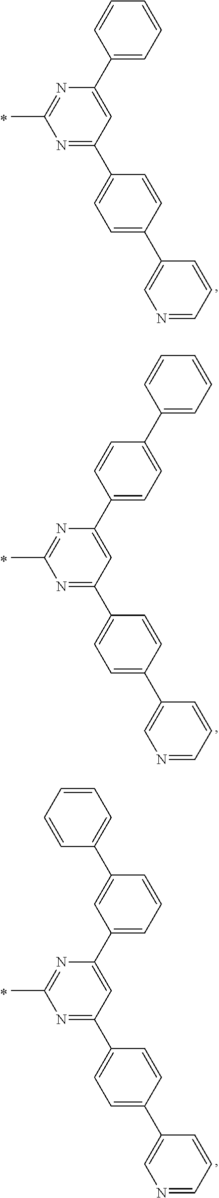

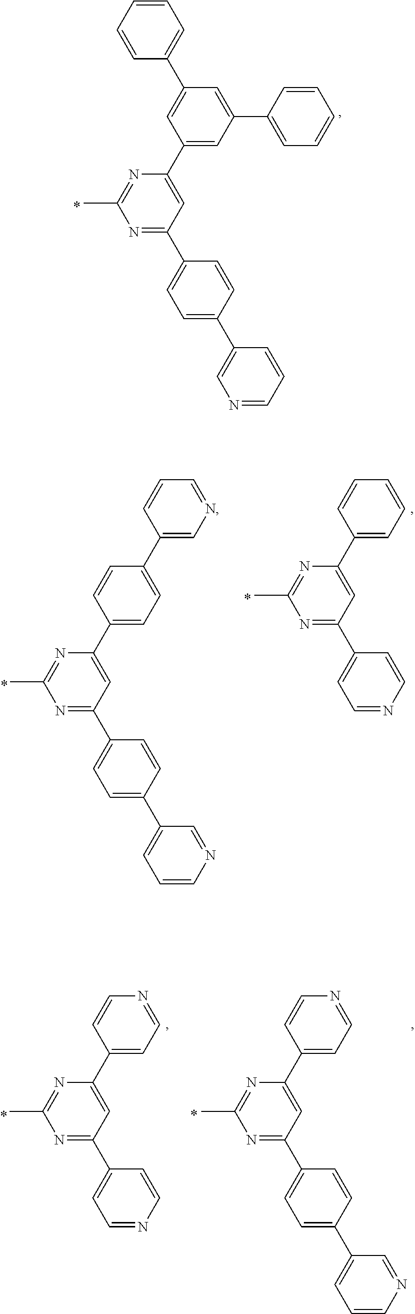 [SMILES: Cc1nc(-c2ccc(-c3cccnc3)cc2)cc(-c2cc(-c3ccccc3)cc(-c3ccccc3)c2)n1 ; Cc1nc(-c2ccc(-c3cccnc3)cc2)cc(-c2ccc(-c3cccnc3)cc2)n1 ; Cc1nc(-c2ccccc2)cc(-c2ccncc2)n1 ; Cc1nc(-c2ccncc2)cc(-c2ccncc2)n1 ; Cc1nc(-c2ccncc2)cc(-c2ccc(-c3cccnc3)cc2)n1]

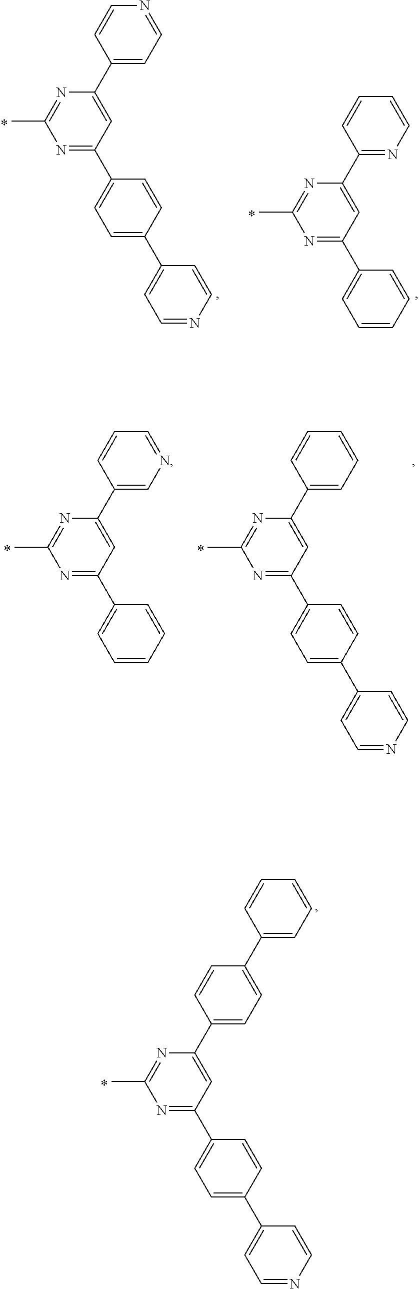 [SMILES: Cc1nc(-c2ccncc2)cc(-c2ccc(-c3ccncc3)cc2)n1 ; Cc1nc(-c2ccccc2)cc(-c2ccccn2)n1 ; Cc1nc(-c2ccccc2)cc(-c2cccnc2)n1 ; Cc1nc(-c2ccccc2)cc(-c2ccc(-c3ccncc3)cc2)n1 ; Cc1nc(-c2ccc(-c3ccccc3)cc2)cc(-c2ccc(-c3ccncc3)cc2)n1]

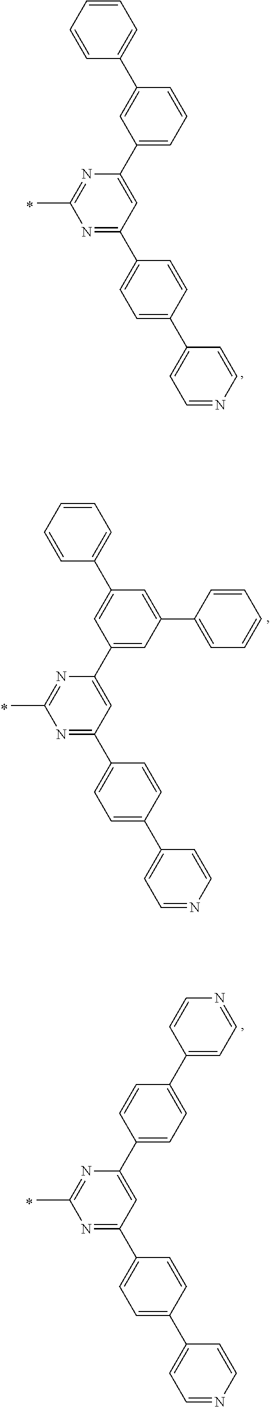 [SMILES: Cc1nc(-c2ccc(-c3ccncc3)cc2)cc(-c2cccc(-c3ccccc3)c2)n1 ; Cc1nc(-c2ccc(-c3ccncc3)cc2)cc(-c2cc(-c3ccccc3)cc(-c3ccccc3)c2)n1 ; Cc1nc(-c2ccc(-c3ccncc3)cc2)cc(-c2ccc(-c3ccncc3)cc2)n1]

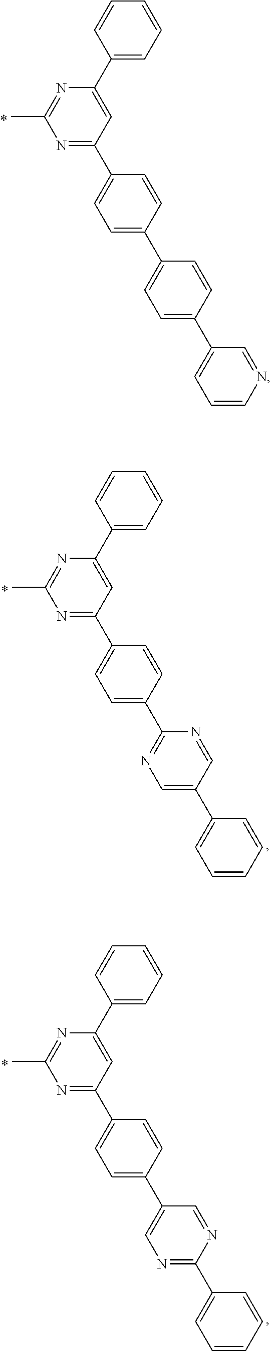 [SMILES: Cc1nc(-c2ccccc2)cc(-c2ccc(-c3ccc(-c4cccnc4)cc3)cc2)n1 ; Cc1nc(-c2ccccc2)cc(-c2ccc(-c3ncc(-c4ccccc4)cn3)cc2)n1 ; Cc1nc(-c2ccccc2)cc(-c2ccc(-c3cnc(-c4ccccc4)nc3)cc2)n1]

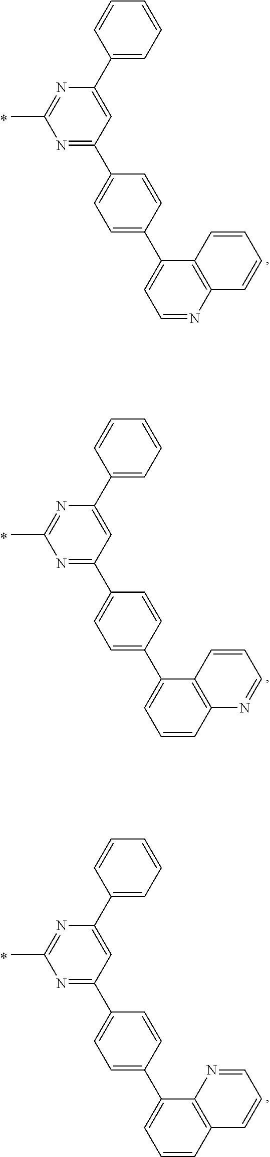 [SMILES: Cc1nc(-c2ccccc2)cc(-c2ccc(-c3ccnc4ccccc34)cc2)n1 ; Cc1nc(-c2ccccc2)cc(-c2ccc(-c3cccc4ncccc34)cc2)n1 ; Cc1nc(-c2ccccc2)cc(-c2ccc(-c3cccc4cccnc34)cc2)n1]

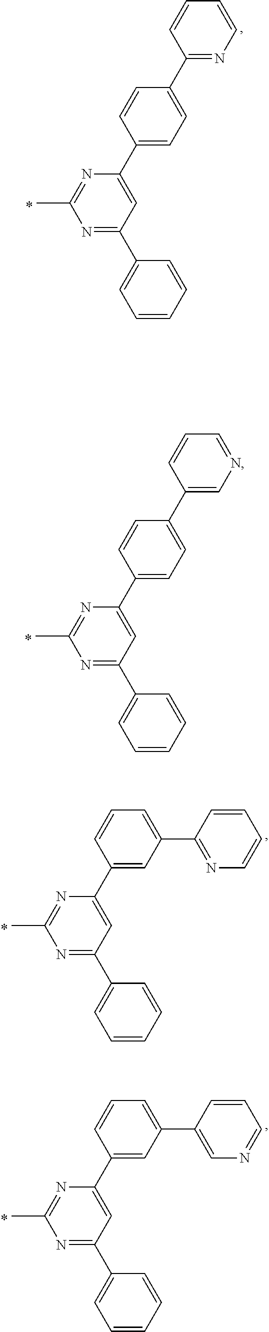 [SMILES: Cc1nc(-c2ccccc2)cc(-c2ccc(-c3ccccn3)cc2)n1 ; Cc1nc(-c2ccccc2)cc(-c2ccc(-c3cccnc3)cc2)n1 ; Cc1nc(-c2ccccc2)cc(-c2cccc(-c3ccccn3)c2)n1 ; Cc1nc(-c2ccccc2)cc(-c2cccc(-c3cccnc3)c2)n1]

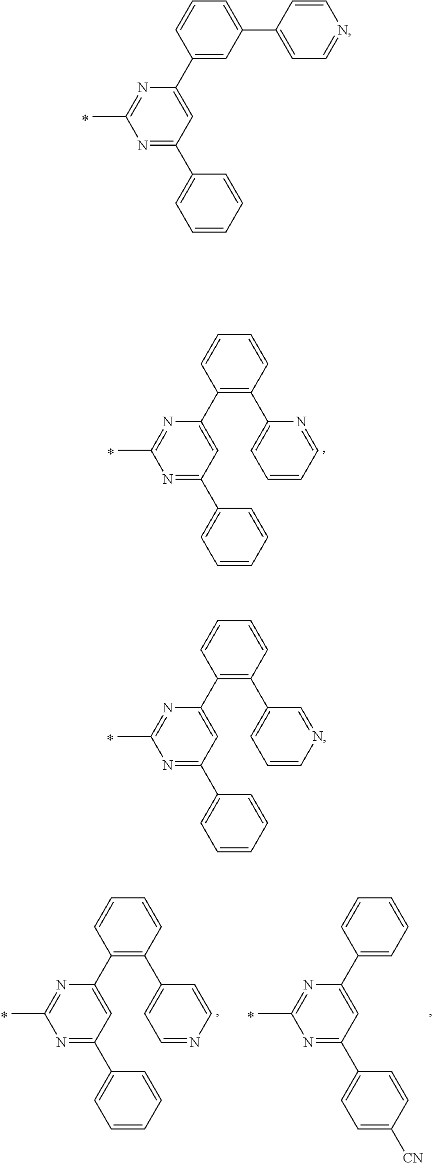 [SMILES: Cc1nc(-c2ccccc2)cc(-c2cccc(-c3ccncc3)c2)n1 ; Cc1nc(-c2ccccc2)cc(-c2ccccc2-c2ccccn2)n1 ; Cc1nc(-c2ccccc2)cc(-c2ccccc2-c2cccnc2)n1 ; Cc1nc(-c2ccccc2)cc(-c2ccccc2-c2ccncc2)n1 ; Cc1nc(-c2ccccc2)cc(-c2ccc(C#N)cc2)n1]

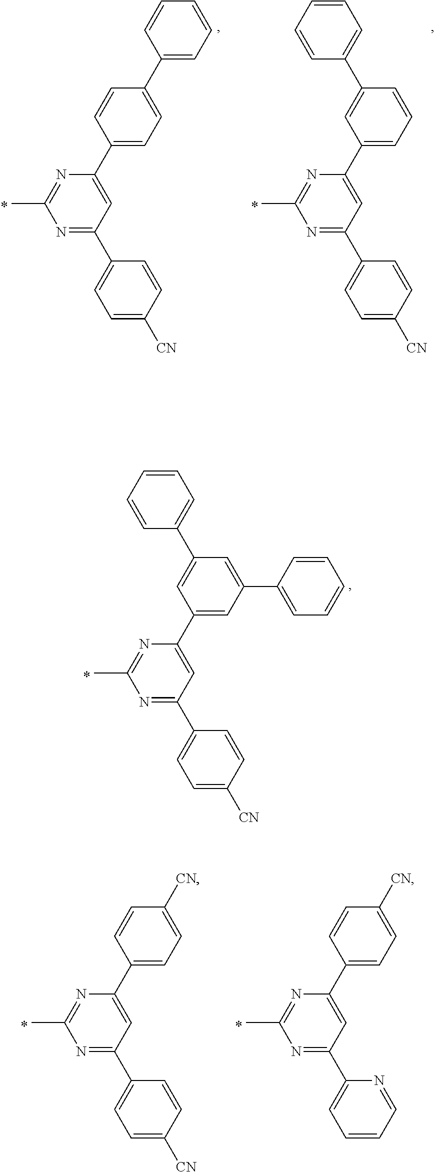 [SMILES: Cc1nc(-c2ccc(C#N)cc2)cc(-c2ccc(-c3ccccc3)cc2)n1 ; Cc1nc(-c2ccc(C#N)cc2)cc(-c2cccc(-c3ccccc3)c2)n1 ; Cc1nc(-c2ccc(C#N)cc2)cc(-c2cc(-c3ccccc3)cc(-c3ccccc3)c2)n1 ; Cc1nc(-c2ccc(C#N)cc2)cc(-c2ccc(C#N)cc2)n1 ; Cc1nc(-c2ccc(C#N)cc2)cc(-c2ccccn2)n1]

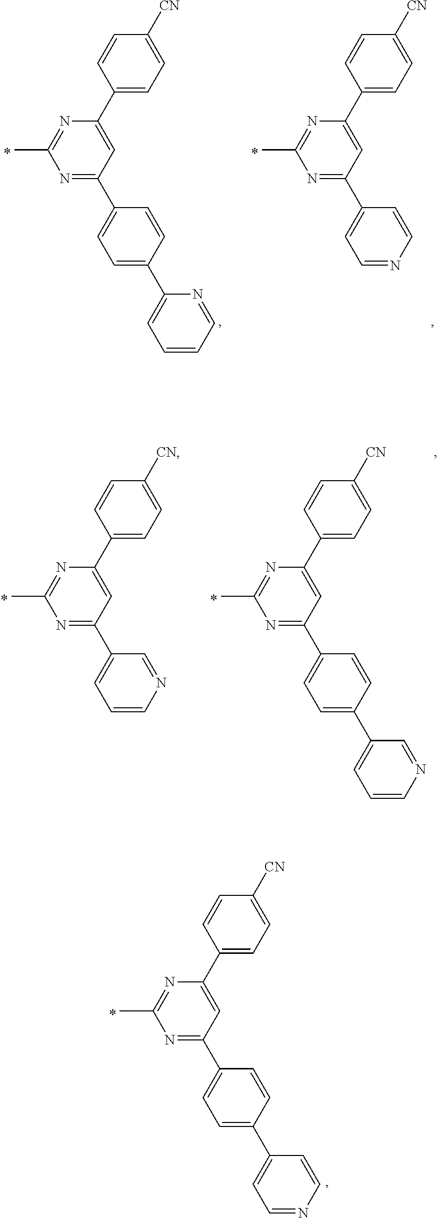 [SMILES: Cc1nc(-c2ccc(C#N)cc2)cc(-c2ccc(-c3ccccn3)cc2)n1 ; Cc1nc(-c2ccncc2)cc(-c2ccc(C#N)cc2)n1 ; Cc1nc(-c2ccc(C#N)cc2)cc(-c2cccnc2)n1 ; Cc1nc(-c2ccc(C#N)cc2)cc(-c2ccc(-c3cccnc3)cc2)n1 ; Cc1nc(-c2ccc(C#N)cc2)cc(-c2ccc(-c3ccncc3)cc2)n1]

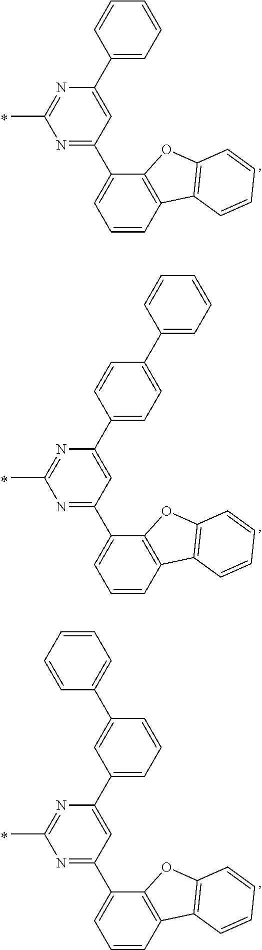

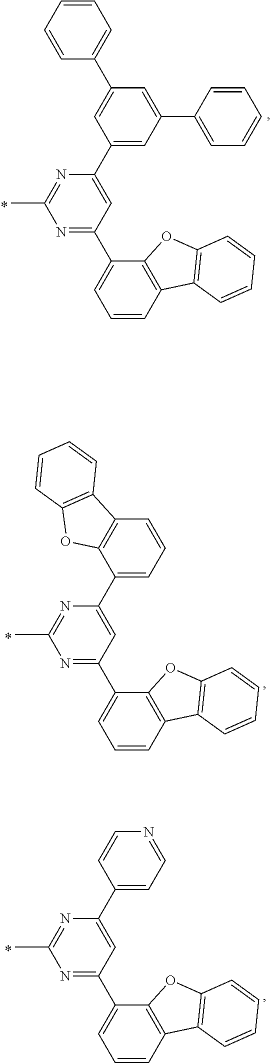 [SMILES: Cc1nc(-c2cc(-c3ccccc3)cc(-c3ccccc3)c2)cc(-c2cccc3c2oc2ccccc23)n1 ; Cc1nc(-c2cccc3c2oc2ccccc23)cc(-c2cccc3c2oc2ccccc23)n1 ; Cc1nc(-c2ccncc2)cc(-c2cccc3c2oc2ccccc23)n1]

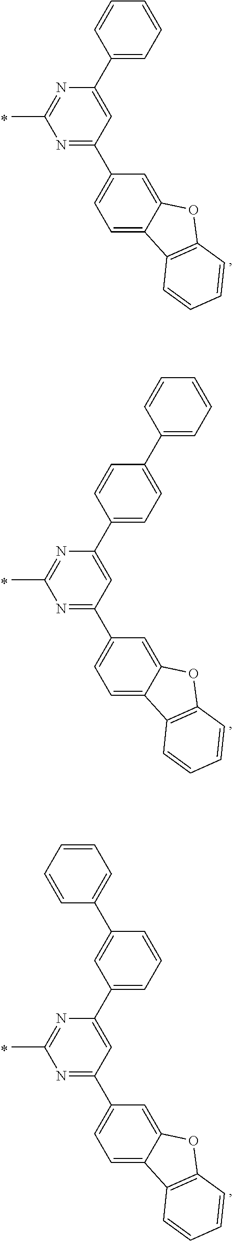 [SMILES: Cc1nc(-c2ccccc2)cc(-c2ccc3c(c2)oc2ccccc23)n1 ; Cc1nc(-c2ccc(-c3ccccc3)cc2)cc(-c2ccc3c(c2)oc2ccccc23)n1 ; Cc1nc(-c2cccc(-c3ccccc3)c2)cc(-c2ccc3c(c2)oc2ccccc23)n1]

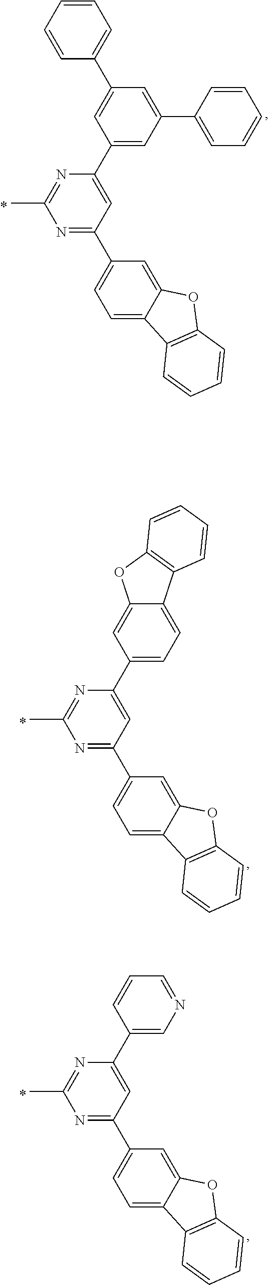 [SMILES: Cc1nc(-c2cc(-c3ccccc3)cc(-c3ccccc3)c2)cc(-c2ccc3c(c2)oc2ccccc23)n1 ; Cc1nc(-c2ccc3c(c2)oc2ccccc23)cc(-c2ccc3c(c2)oc2ccccc23)n1 ; Cc1nc(-c2cccnc2)cc(-c2ccc3c(c2)oc2ccccc23)n1]

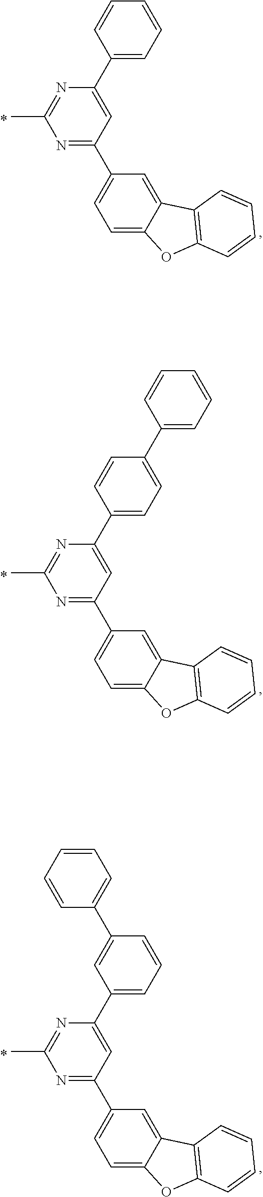 [SMILES: Cc1nc(-c2ccccc2)cc(-c2ccc3oc4ccccc4c3c2)n1 ; Cc1nc(-c2ccc(-c3ccccc3)cc2)cc(-c2ccc3oc4ccccc4c3c2)n1 ; Cc1nc(-c2cccc(-c3ccccc3)c2)cc(-c2ccc3oc4ccccc4c3c2)n1]

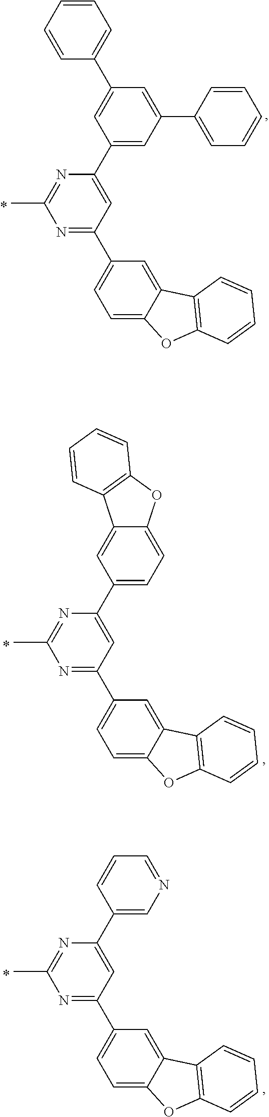 [SMILES: Cc1nc(-c2cc(-c3ccccc3)cc(-c3ccccc3)c2)cc(-c2ccc3oc4ccccc4c3c2)n1 ; Cc1nc(-c2ccc3oc4ccccc4c3c2)cc(-c2ccc3oc4ccccc4c3c2)n1 ; Cc1nc(-c2cccnc2)cc(-c2ccc3oc4ccccc4c3c2)n1]

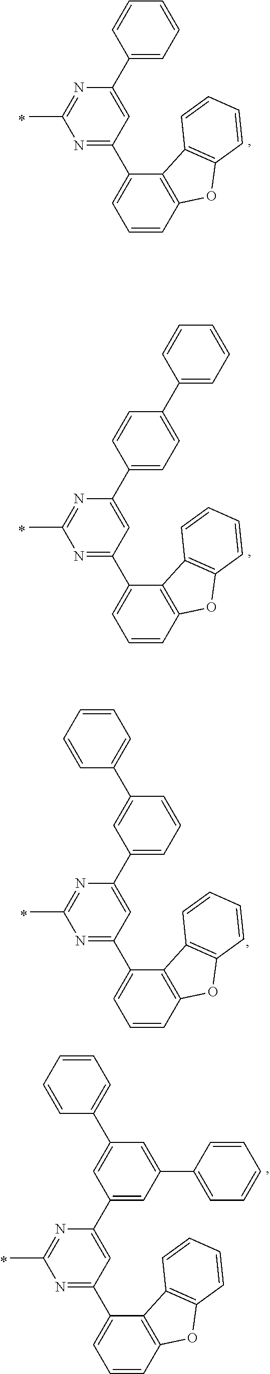

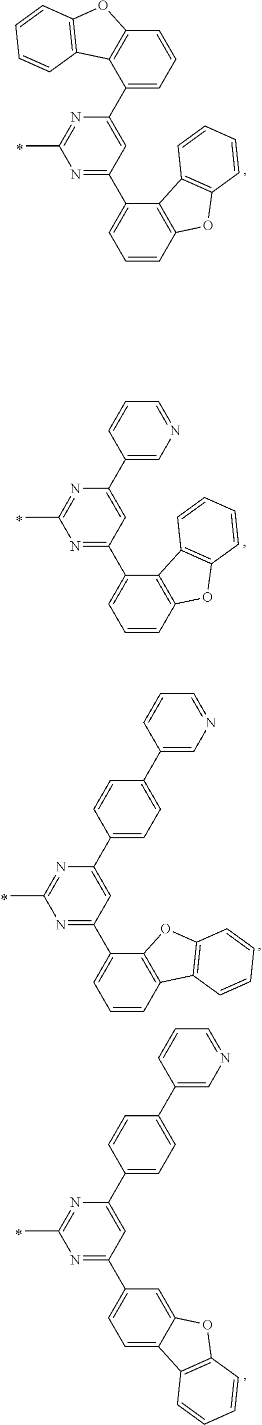

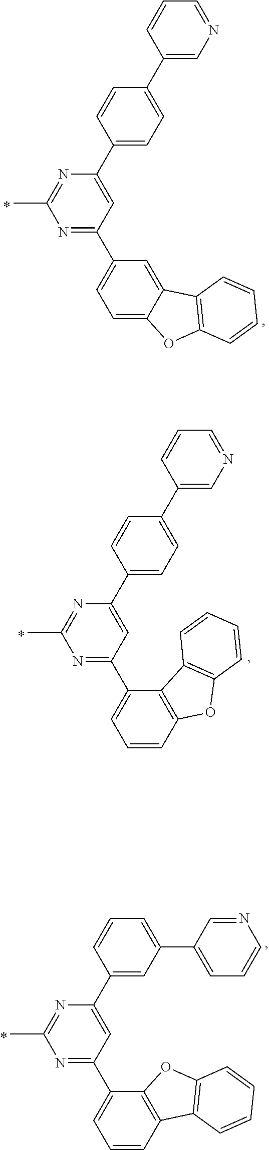 [SMILES: Cc1nc(-c2ccc(-c3cccnc3)cc2)cc(-c2ccc3oc4ccccc4c3c2)n1 ; Cc1nc(-c2ccc(-c3cccnc3)cc2)cc(-c2cccc3oc4ccccc4c23)n1 ; Cc1nc(-c2cccc(-c3cccnc3)c2)cc(-c2cccc3c2oc2ccccc23)n1]

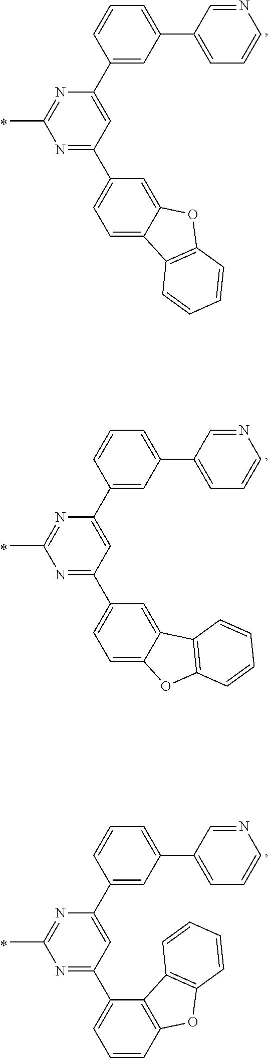 [SMILES: Cc1nc(-c2cccc(-c3cccnc3)c2)cc(-c2ccc3c(c2)oc2ccccc23)n1 ; Cc1nc(-c2cccc(-c3cccnc3)c2)cc(-c2ccc3oc4ccccc4c3c2)n1 ; Cc1nc(-c2cccc(-c3cccnc3)c2)cc(-c2cccc3oc4ccccc4c23)n1]

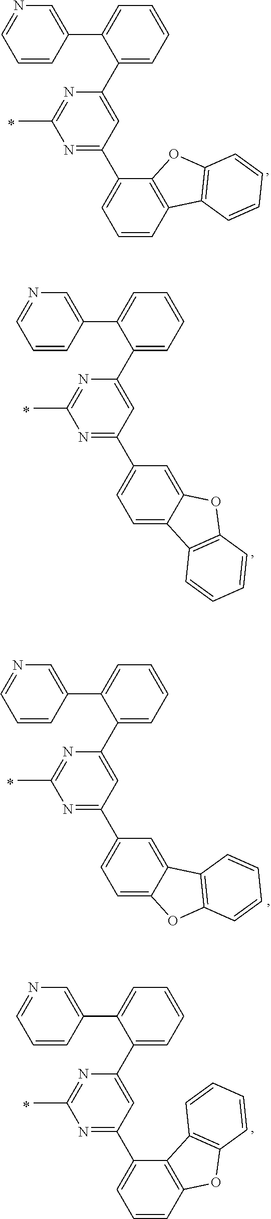 [SMILES: Cc1nc(-c2ccccc2-c2cccnc2)cc(-c2cccc3c2oc2ccccc23)n1 ; Cc1nc(-c2ccc3c(c2)oc2ccccc23)cc(-c2ccccc2-c2cccnc2)n1 ; Cc1nc(-c2ccc3oc4ccccc4c3c2)cc(-c2ccccc2-c2cccnc2)n1 ; Cc1nc(-c2ccccc2-c2cccnc2)cc(-c2cccc3oc4ccccc4c23)n1]

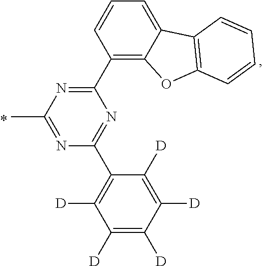 [SMILES: [2H]c1c([2H])c([2H])c(-c2nc(C)nc(-c3cccc4c3oc3ccccc34)n2)c([2H])c1[2H]]

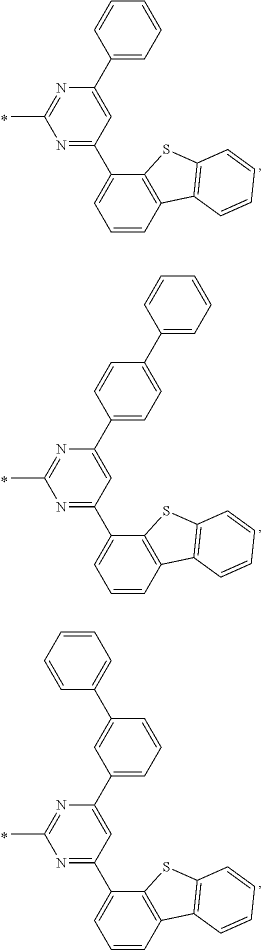

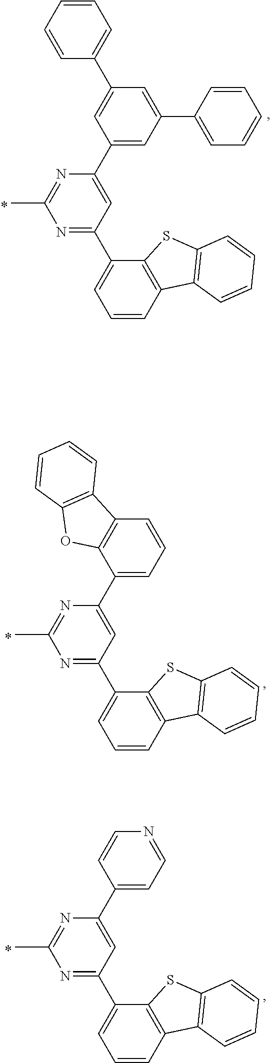 [SMILES: Cc1nc(-c2cc(-c3ccccc3)cc(-c3ccccc3)c2)cc(-c2cccc3c2sc2ccccc23)n1 ; Cc1nc(-c2cccc3c2oc2ccccc23)cc(-c2cccc3c2sc2ccccc23)n1 ; Cc1nc(-c2ccncc2)cc(-c2cccc3c2sc2ccccc23)n1]

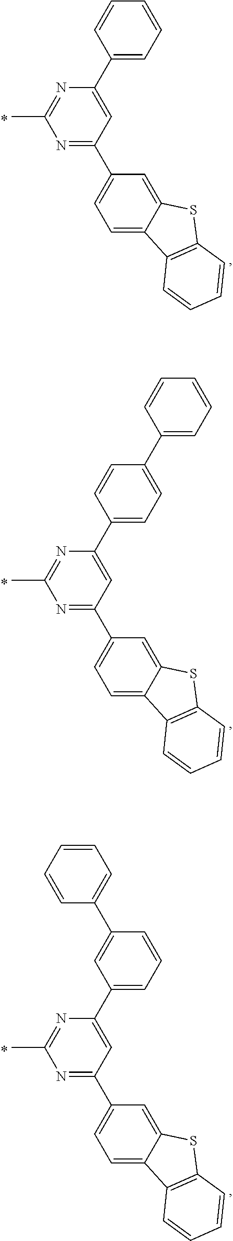 [SMILES: Cc1nc(-c2ccccc2)cc(-c2ccc3c(c2)sc2ccccc23)n1 ; Cc1nc(-c2ccc(-c3ccccc3)cc2)cc(-c2ccc3c(c2)sc2ccccc23)n1 ; Cc1nc(-c2cccc(-c3ccccc3)c2)cc(-c2ccc3c(c2)sc2ccccc23)n1]

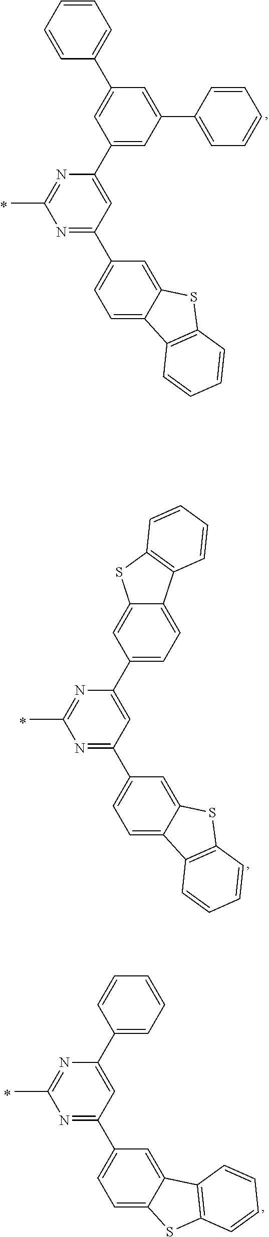 [SMILES: Cc1nc(-c2cc(-c3ccccc3)cc(-c3ccccc3)c2)cc(-c2ccc3c(c2)sc2ccccc23)n1 ; Cc1nc(-c2ccc3c(c2)sc2ccccc23)cc(-c2ccc3c(c2)sc2ccccc23)n1 ; Cc1nc(-c2ccccc2)cc(-c2ccc3sc4ccccc4c3c2)n1]

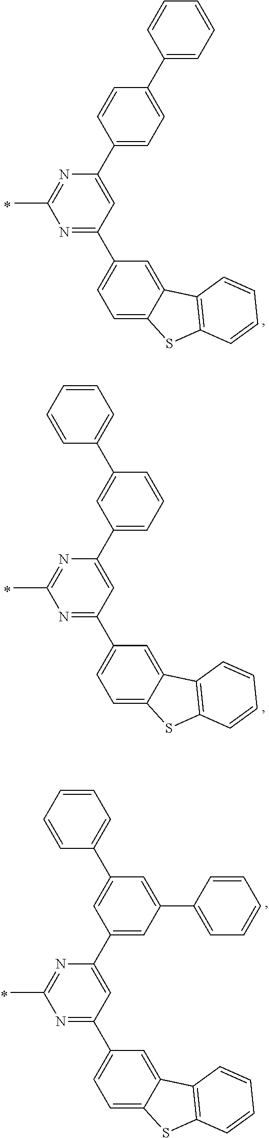

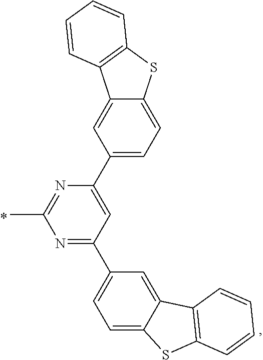 [SMILES: Cc1nc(-c2ccc3sc4ccccc4c3c2)cc(-c2ccc3sc4ccccc4c3c2)n1]

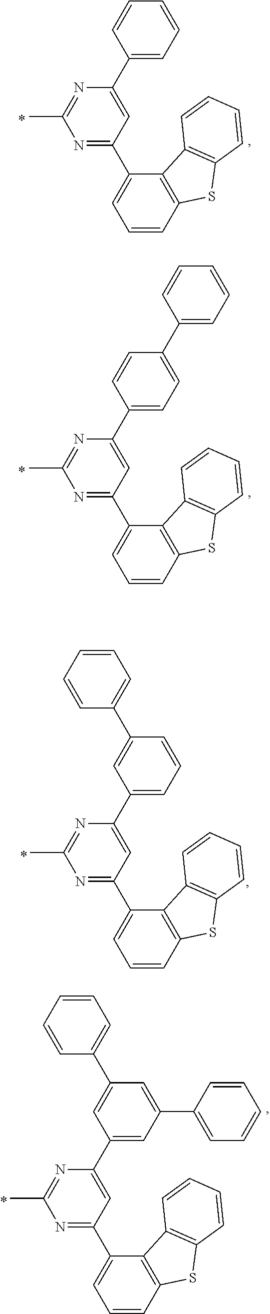

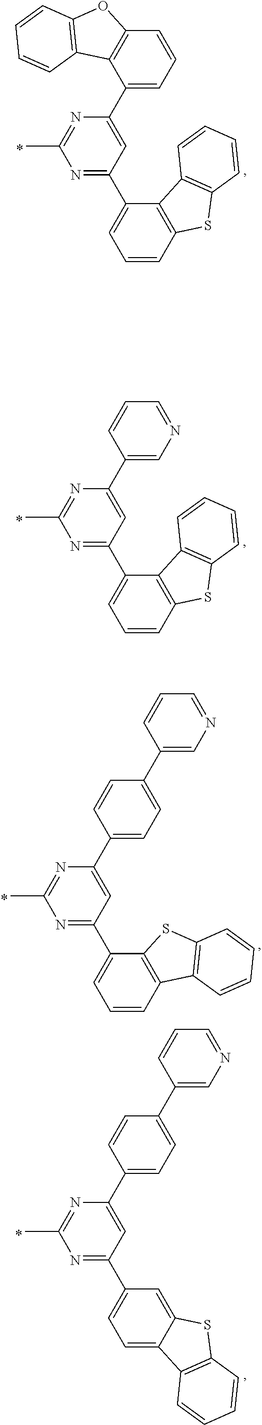

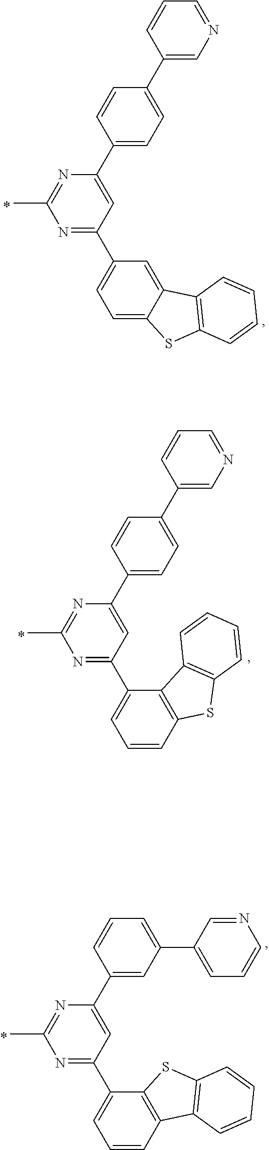 [SMILES: Cc1nc(-c2ccc(-c3cccnc3)cc2)cc(-c2ccc3sc4ccccc4c3c2)n1 ; Cc1nc(-c2ccc(-c3cccnc3)cc2)cc(-c2cccc3sc4ccccc4c23)n1 ; Cc1nc(-c2cccc(-c3cccnc3)c2)cc(-c2cccc3c2sc2ccccc23)n1]

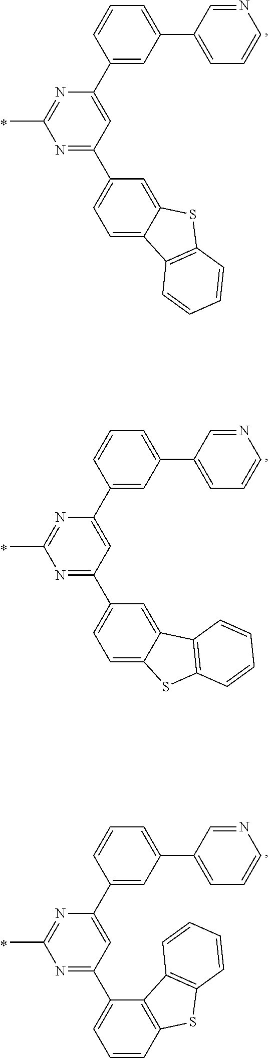 [SMILES: Cc1nc(-c2cccc(-c3cccnc3)c2)cc(-c2ccc3c(c2)sc2ccccc23)n1 ; Cc1nc(-c2cccc(-c3cccnc3)c2)cc(-c2ccc3sc4ccccc4c3c2)n1 ; Cc1nc(-c2cccc(-c3cccnc3)c2)cc(-c2cccc3sc4ccccc4c23)n1]

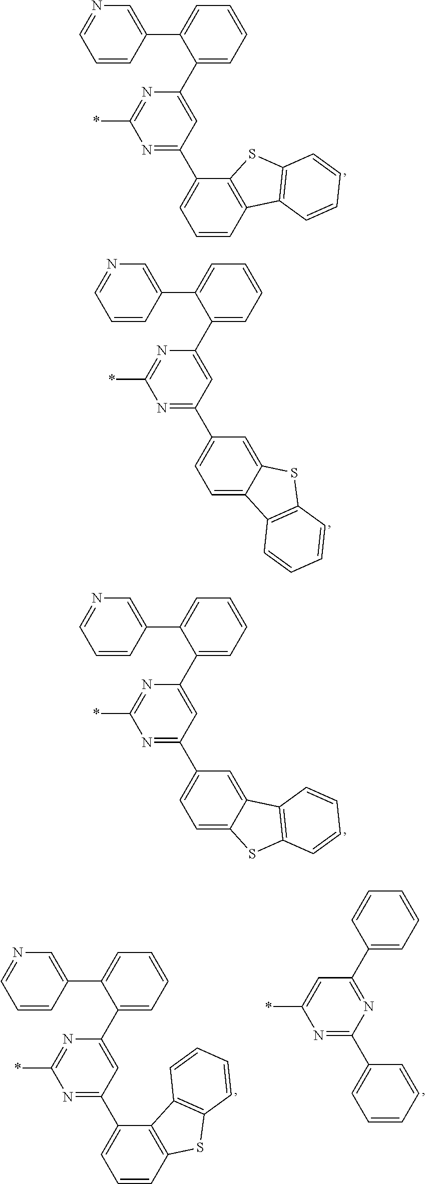

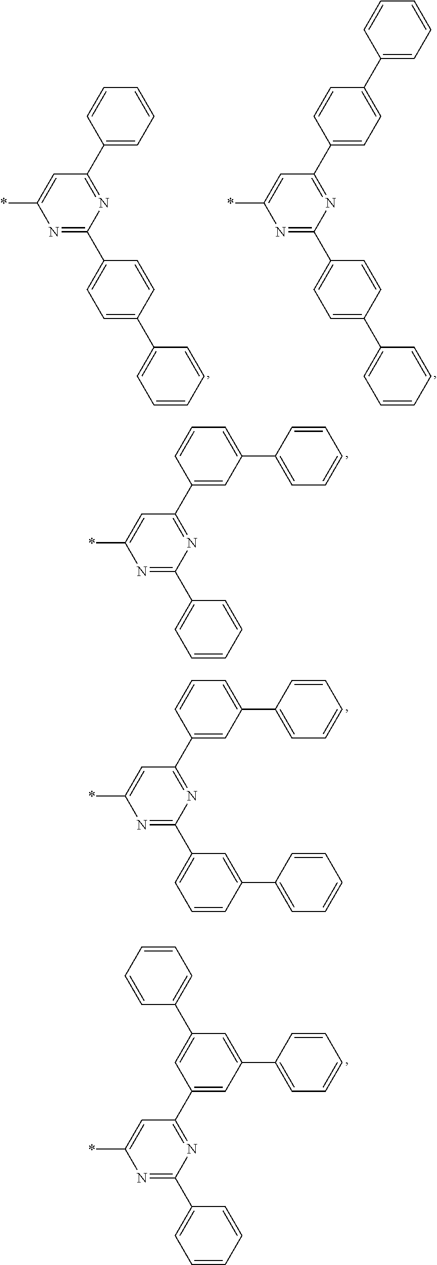

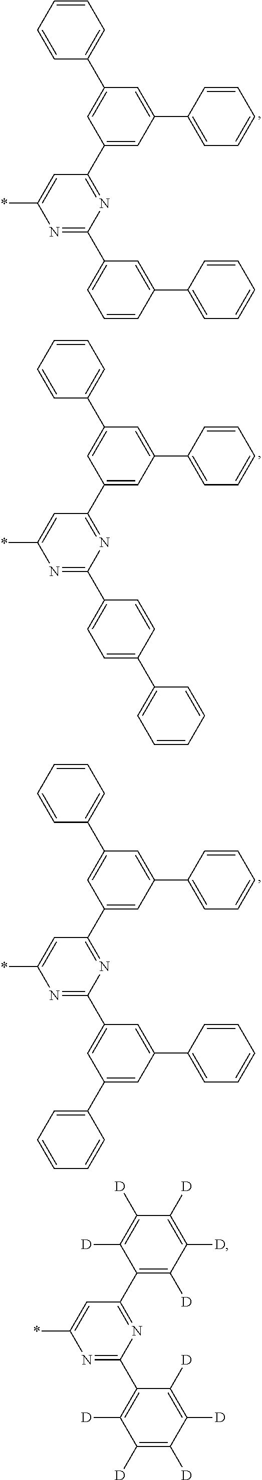

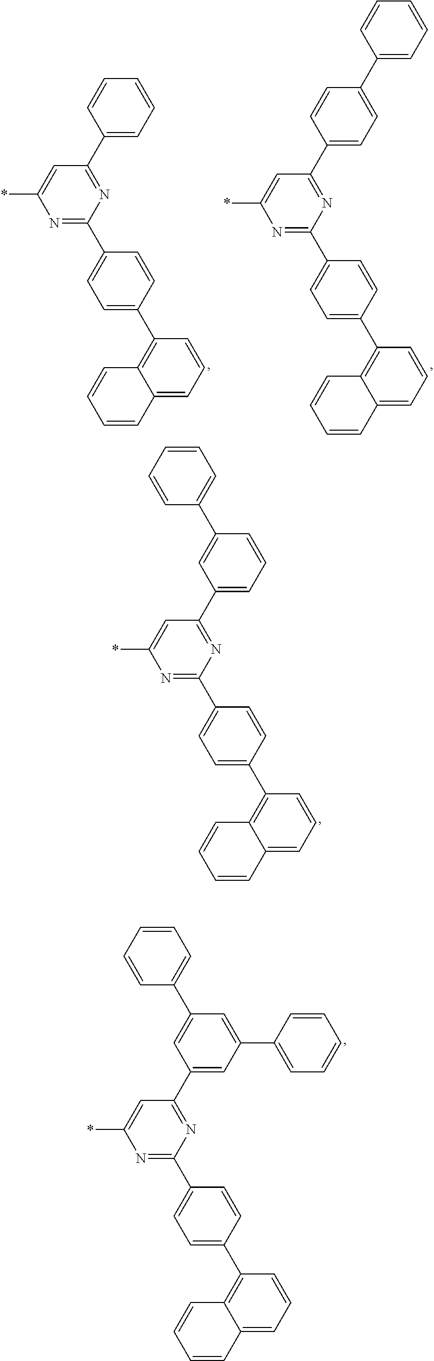

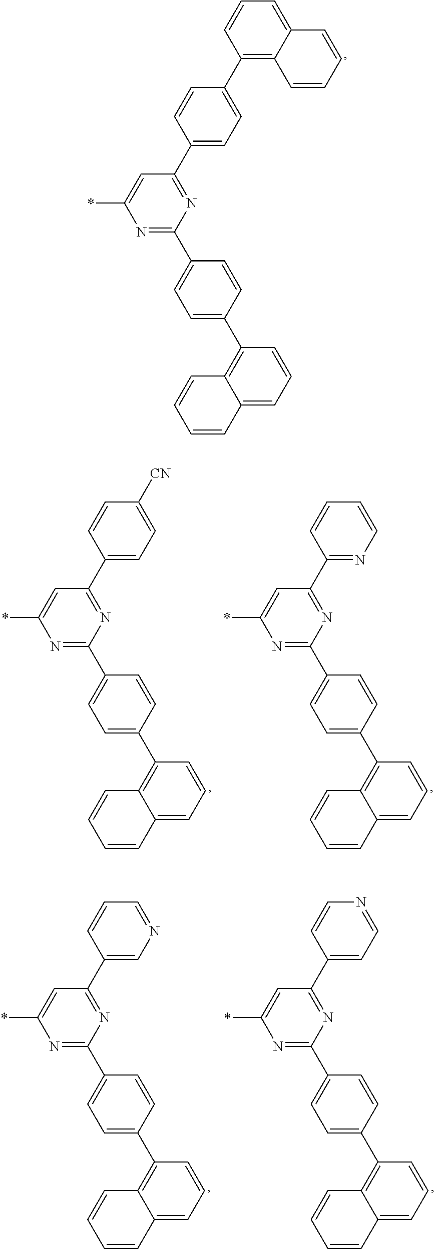 [SMILES: *c1cc(-c2ccc(-c3cccc4ccccc34)cc2)nc(-c2ccc(-c3cccc4ccccc34)cc2)n1 ; *c1cc(-c2ccc(C#N)cc2)nc(-c2ccc(-c3cccc4ccccc34)cc2)n1 ; *c1cc(-c2ccccn2)nc(-c2ccc(-c3cccc4ccccc34)cc2)n1 ; *c1cc(-c2cccnc2)nc(-c2ccc(-c3cccc4ccccc34)cc2)n1 ; *c1cc(-c2ccncc2)nc(-c2ccc(-c3cccc4ccccc34)cc2)n1]

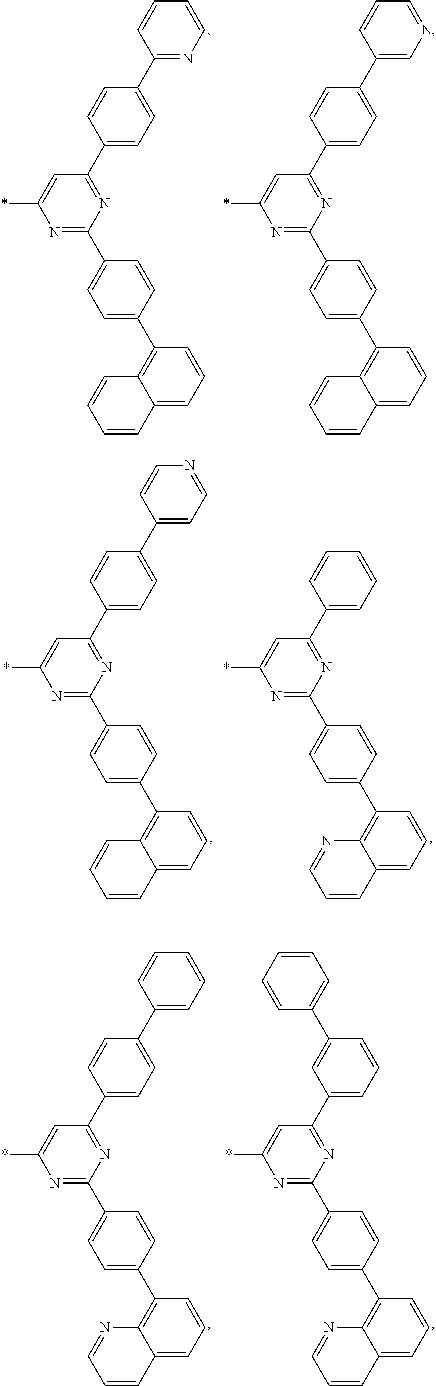

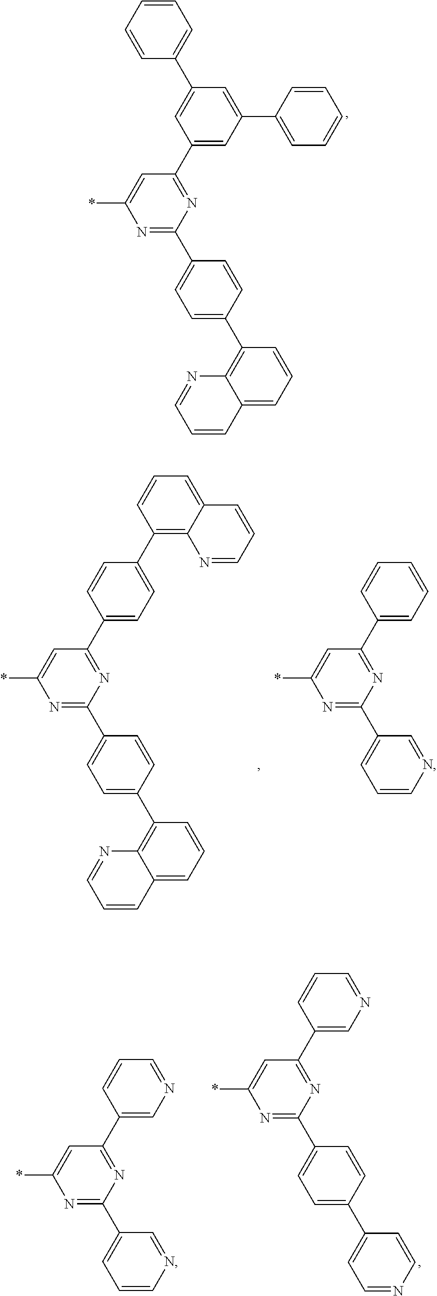

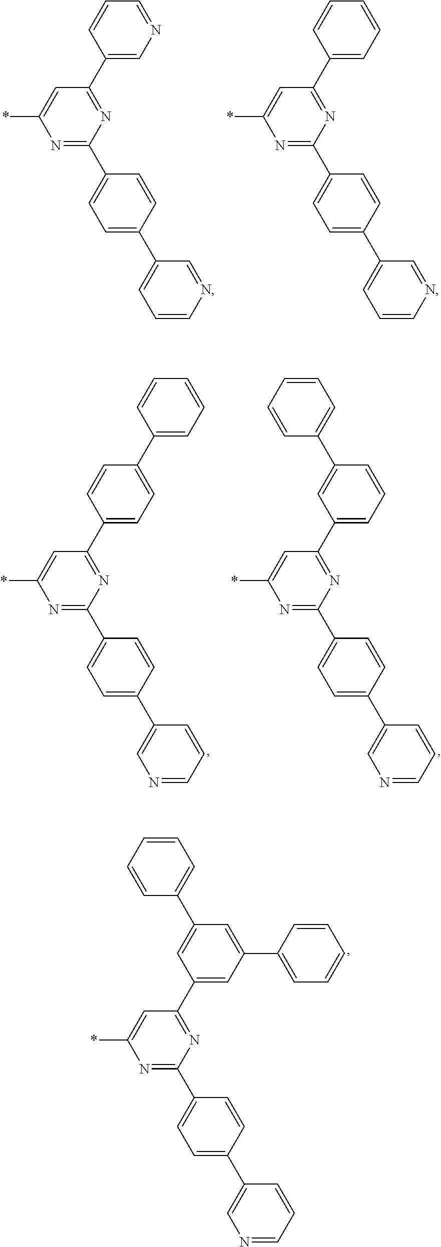 [SMILES: *c1cc(-c2cccnc2)nc(-c2ccc(-c3cccnc3)cc2)n1 ; *c1cc(-c2ccccc2)nc(-c2ccc(-c3cccnc3)cc2)n1 ; *c1cc(-c2ccc(-c3ccccc3)cc2)nc(-c2ccc(-c3cccnc3)cc2)n1 ; *c1cc(-c2cccc(-c3ccccc3)c2)nc(-c2ccc(-c3cccnc3)cc2)n1 ; *c1cc(-c2cc(-c3ccccc3)cc(-c3ccccc3)c2)nc(-c2ccc(-c3cccnc3)cc2)n1]

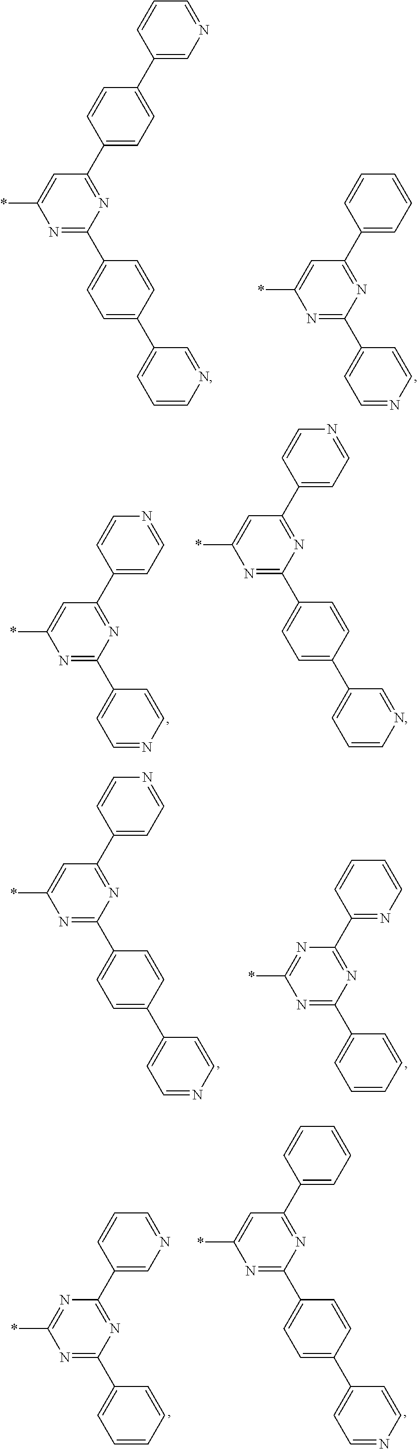

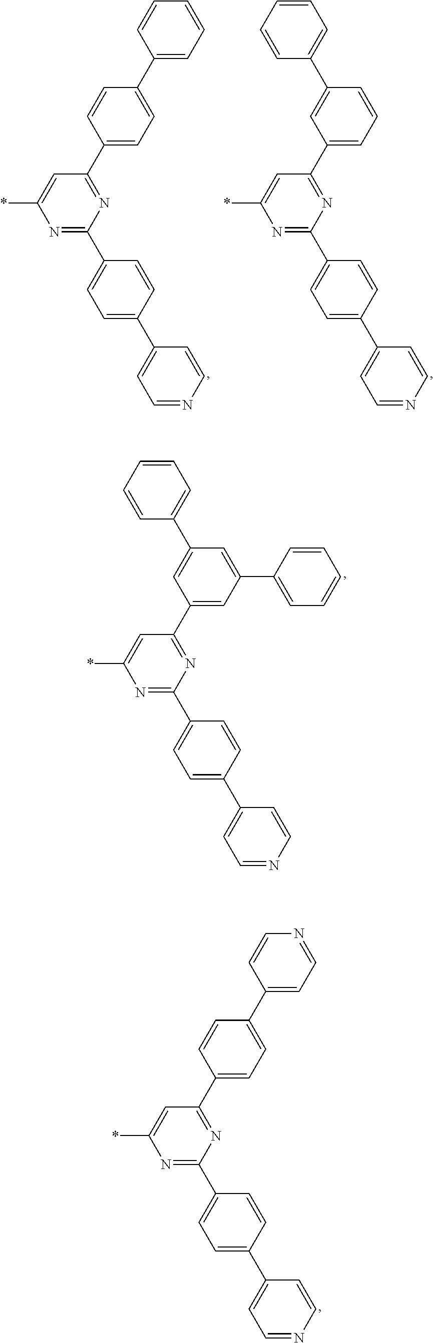 [SMILES: *c1cc(-c2ccc(-c3ccccc3)cc2)nc(-c2ccc(-c3ccncc3)cc2)n1 ; *c1cc(-c2cccc(-c3ccccc3)c2)nc(-c2ccc(-c3ccncc3)cc2)n1 ; *c1cc(-c2cc(-c3ccccc3)cc(-c3ccccc3)c2)nc(-c2ccc(-c3ccncc3)cc2)n1 ; *c1cc(-c2ccc(-c3ccncc3)cc2)nc(-c2ccc(-c3ccncc3)cc2)n1]

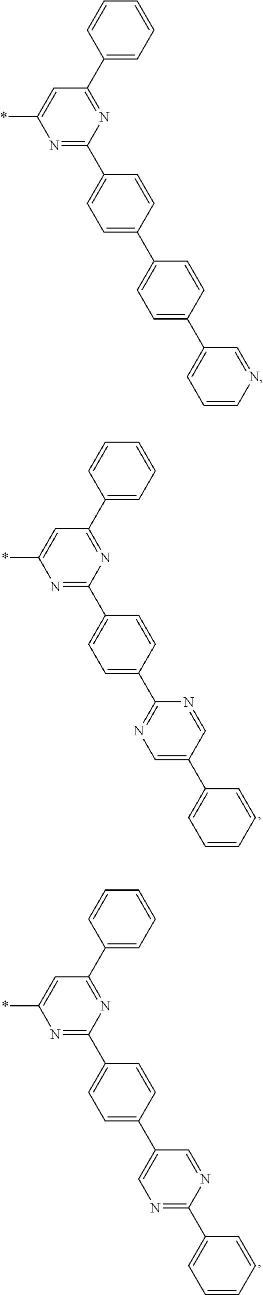 [SMILES: *c1cc(-c2ccccc2)nc(-c2ccc(-c3ccc(-c4cccnc4)cc3)cc2)n1 ; *c1cc(-c2ccccc2)nc(-c2ccc(-c3ncc(-c4ccccc4)cn3)cc2)n1 ; *c1cc(-c2ccccc2)nc(-c2ccc(-c3cnc(-c4ccccc4)nc3)cc2)n1]

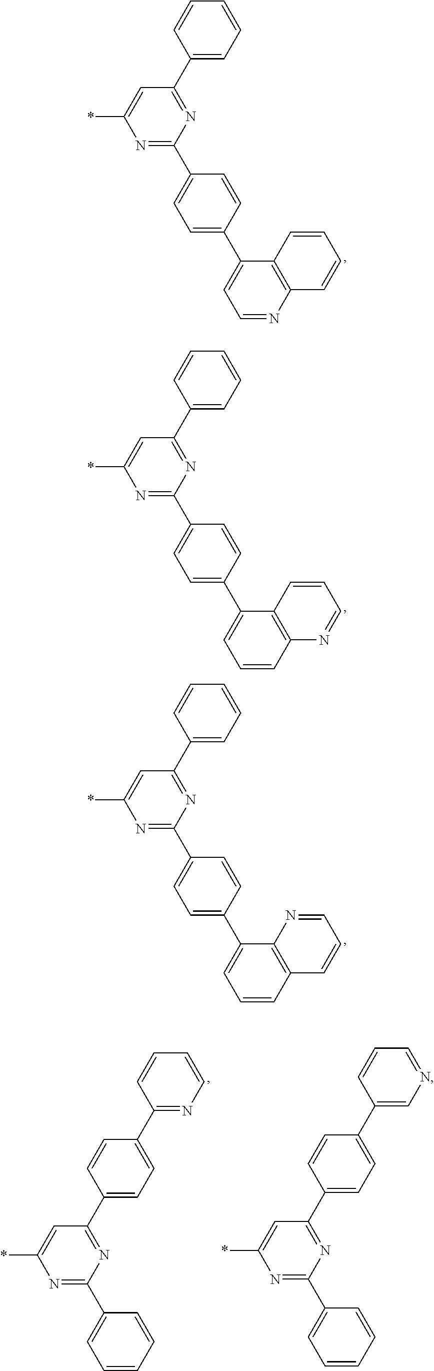

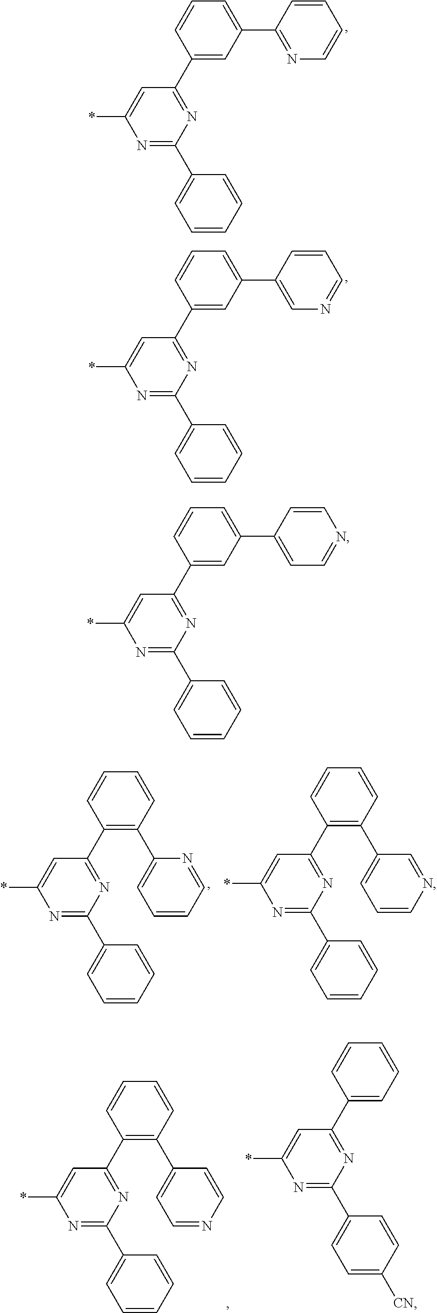

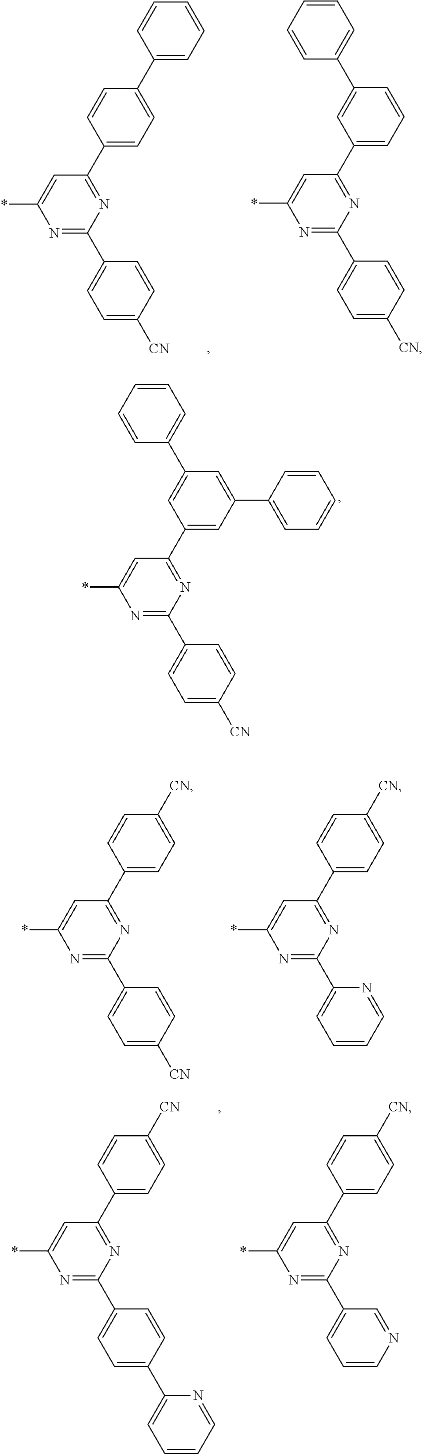

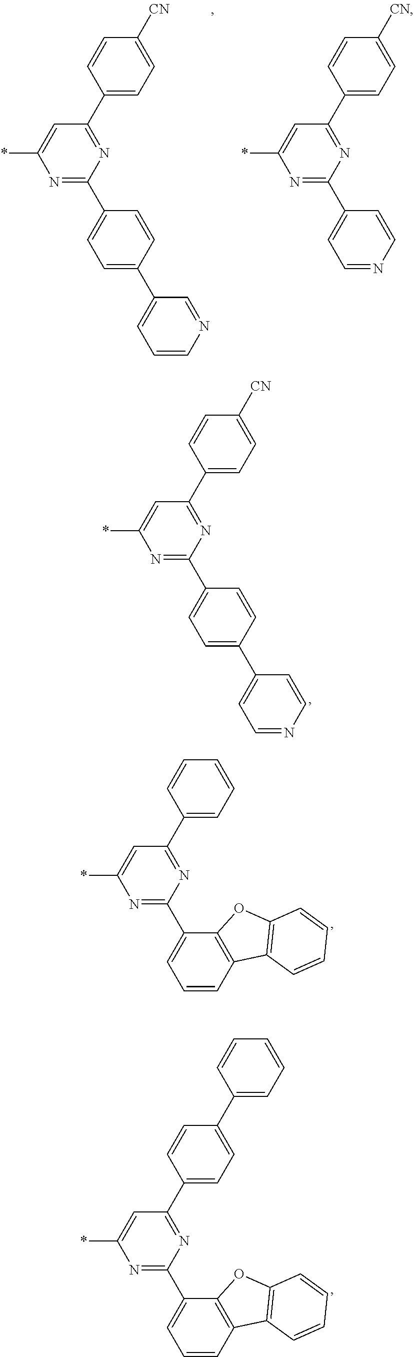 [SMILES: *c1cc(-c2ccc(C#N)cc2)nc(-c2ccc(-c3cccnc3)cc2)n1 ; *c1cc(-c2ccc(C#N)cc2)nc(-c2ccncc2)n1 ; *c1cc(-c2ccc(C#N)cc2)nc(-c2ccc(-c3ccncc3)cc2)n1 ; *c1cc(-c2ccccc2)nc(-c2cccc3c2oc2ccccc23)n1 ; *c1cc(-c2ccc(-c3ccccc3)cc2)nc(-c2cccc3c2oc2ccccc23)n1]

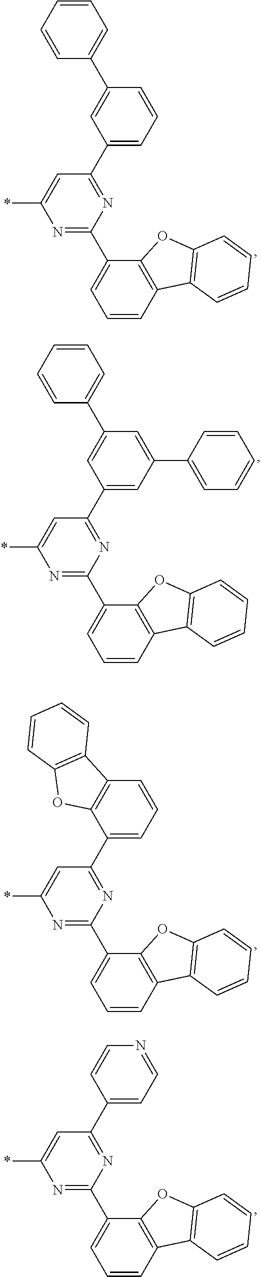

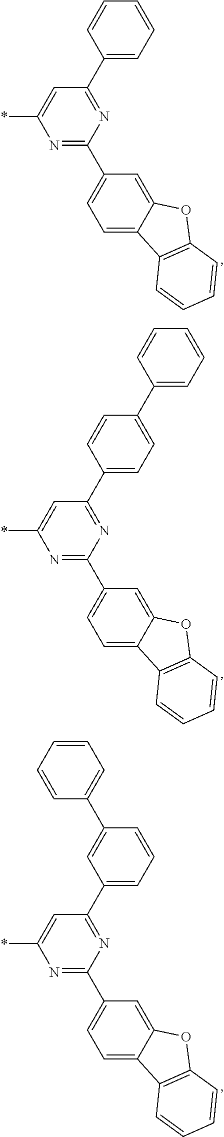

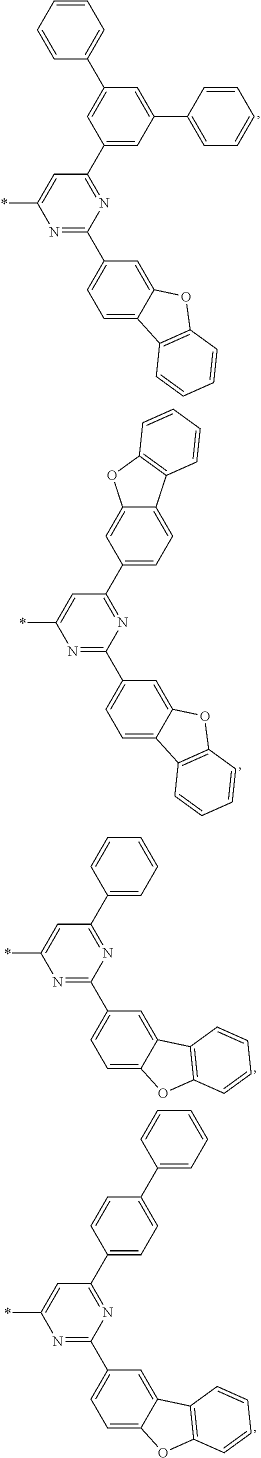

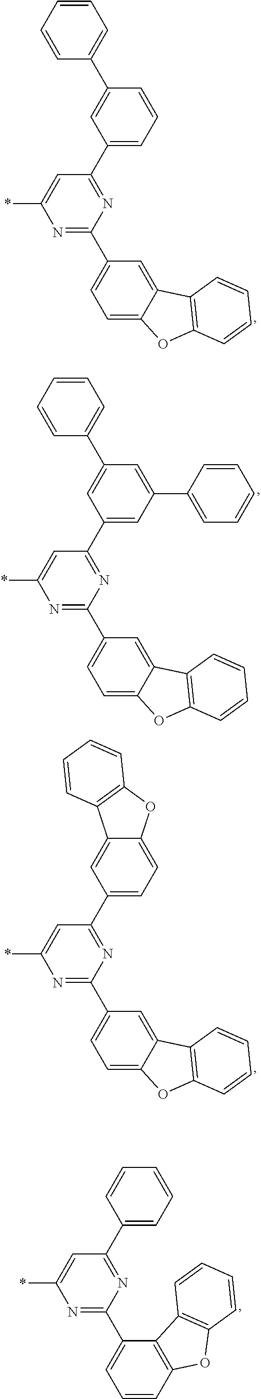

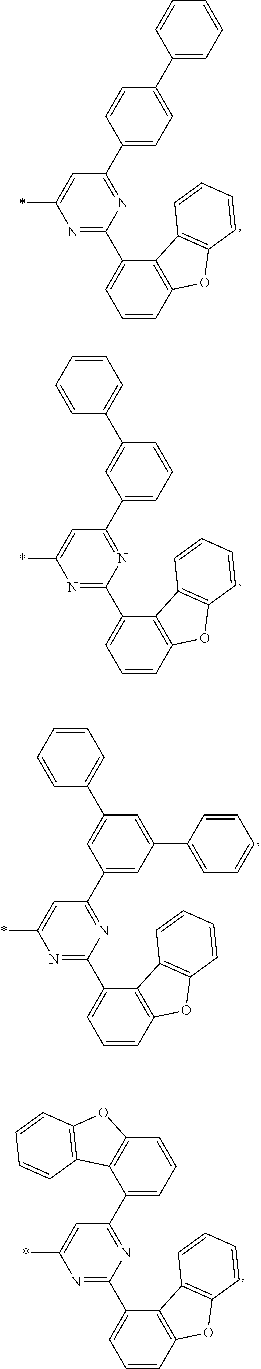

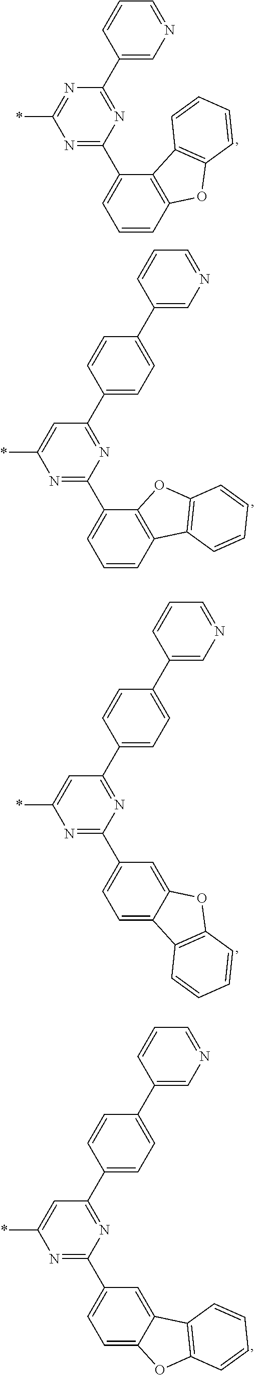

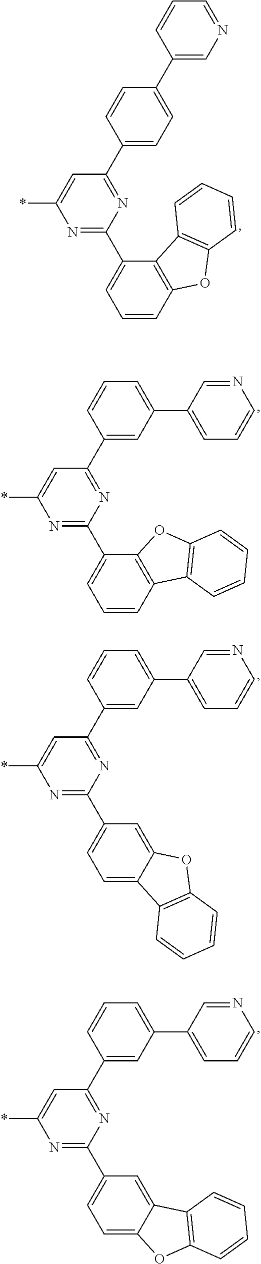

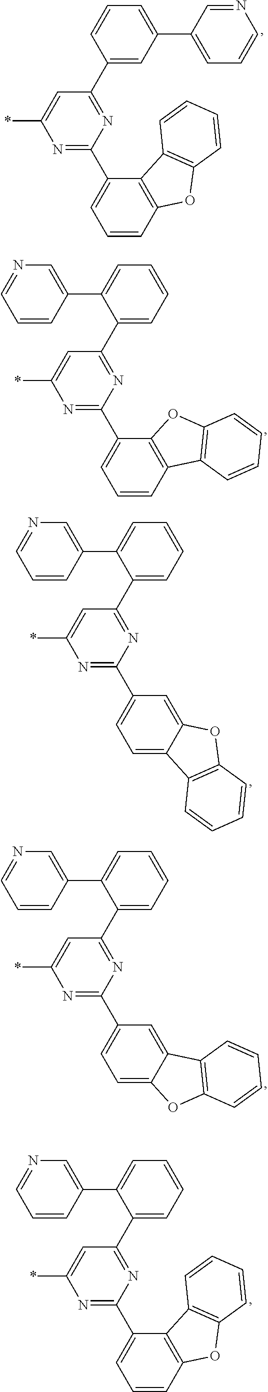

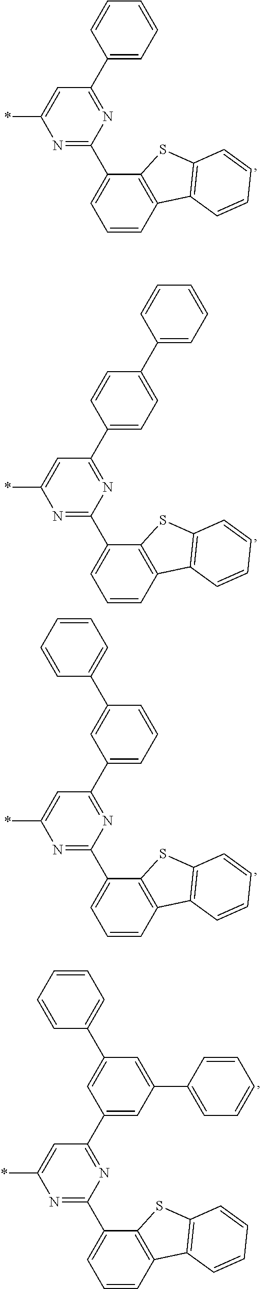

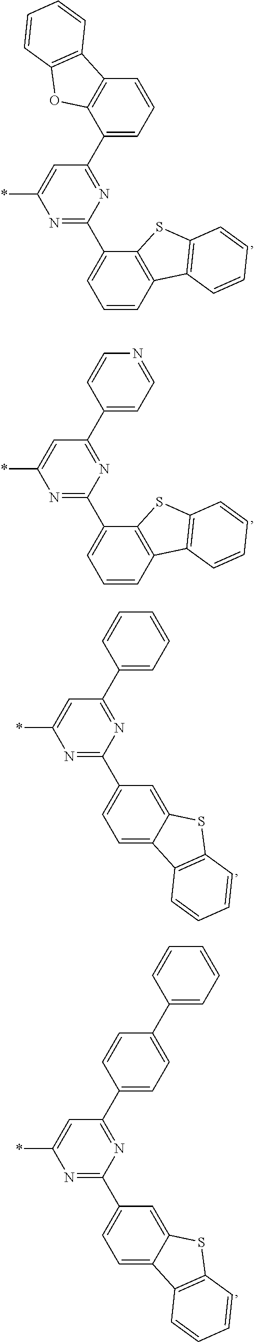

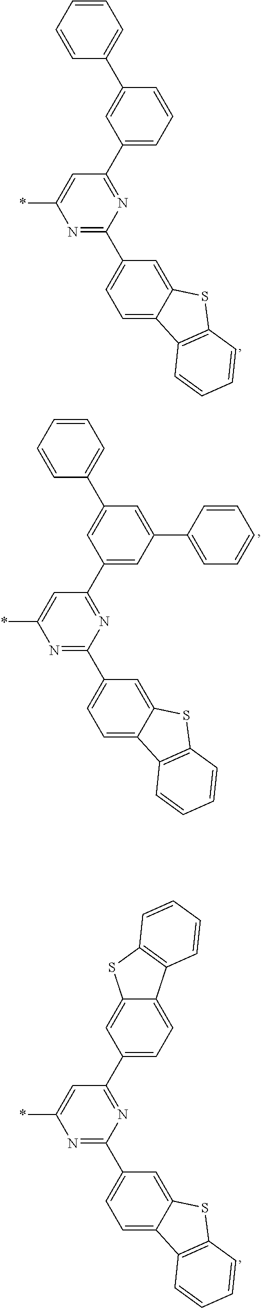 [SMILES: *c1cc(-c2cccc(-c3ccccc3)c2)nc(-c2ccc3c(c2)sc2ccccc23)n1 ; *c1cc(-c2cc(-c3ccccc3)cc(-c3ccccc3)c2)nc(-c2ccc3c(c2)sc2ccccc23)n1 ; *c1cc(-c2ccc3c(c2)sc2ccccc23)nc(-c2ccc3c(c2)sc2ccccc23)n1]

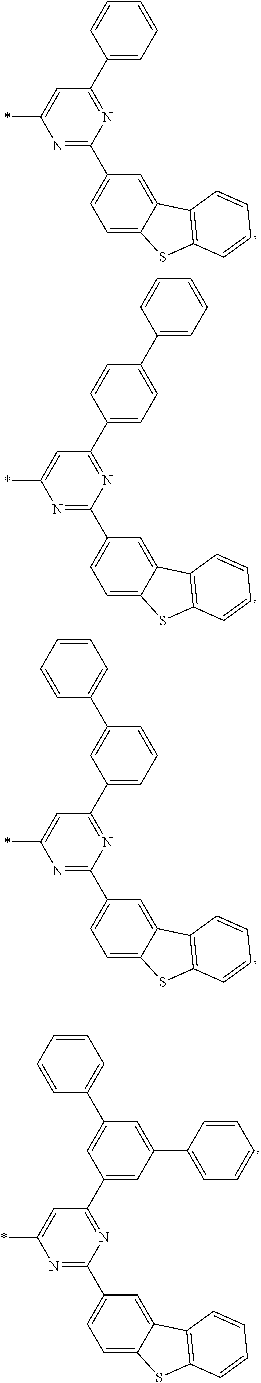

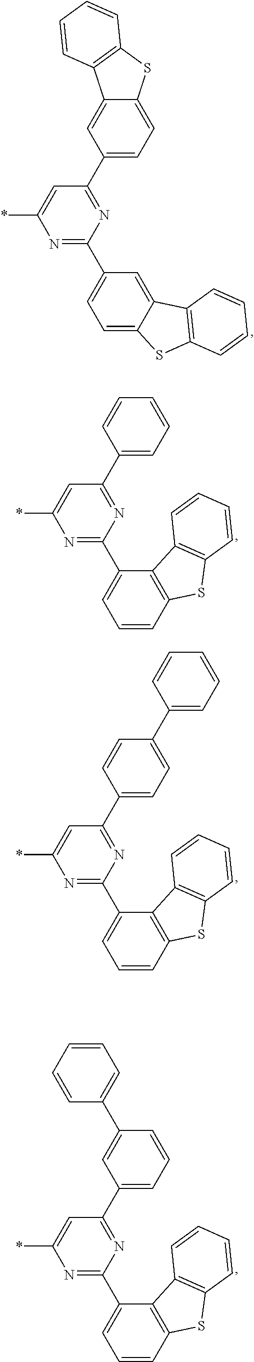

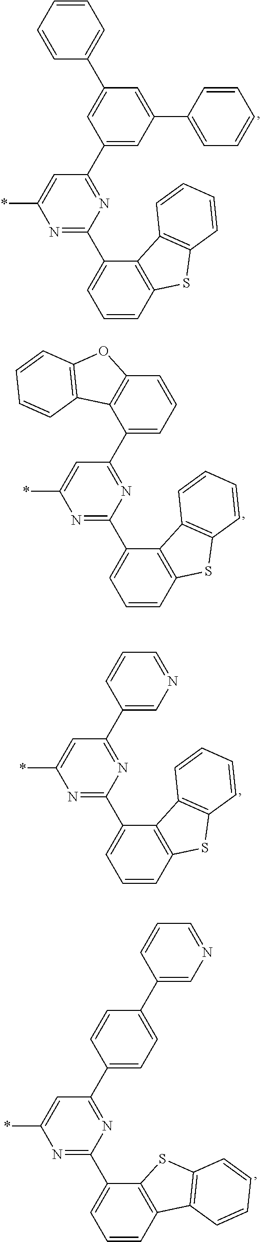

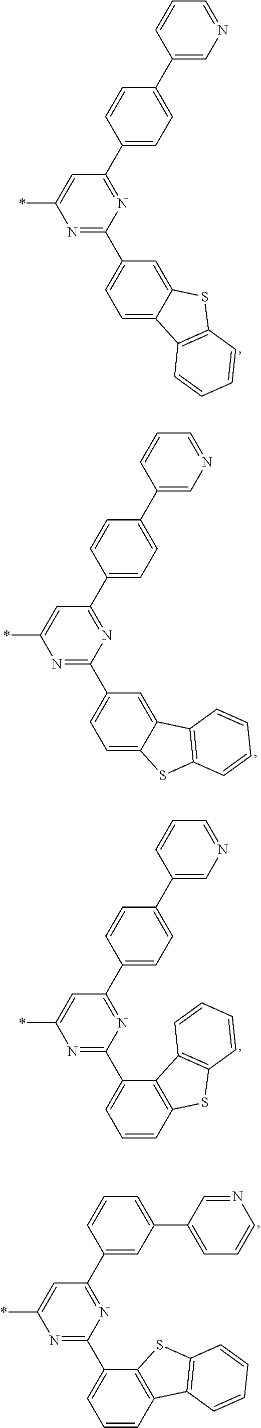

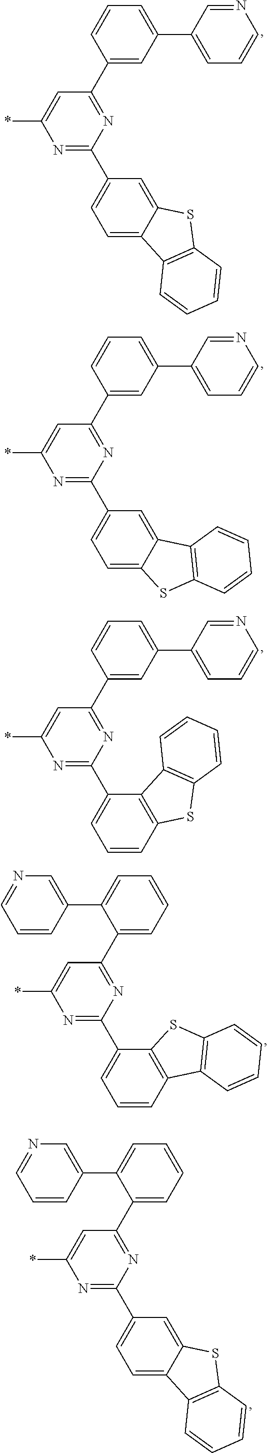

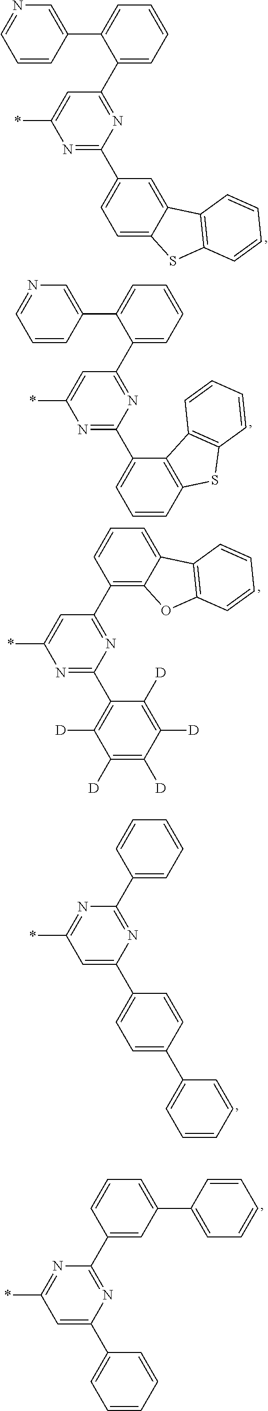

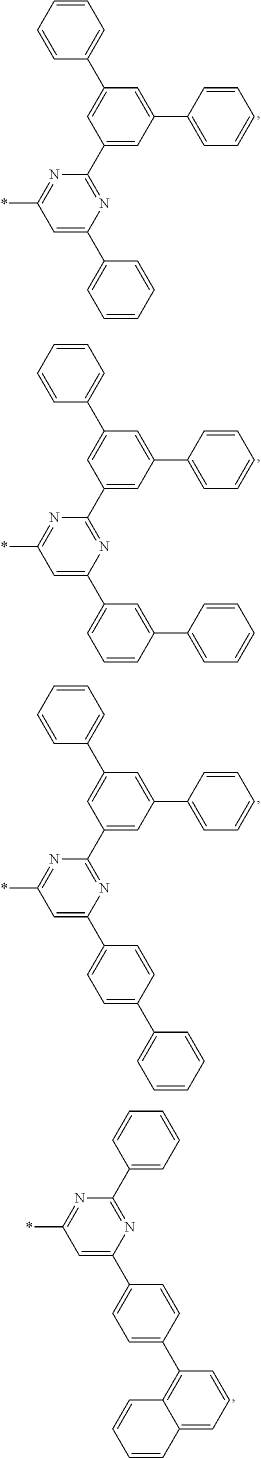

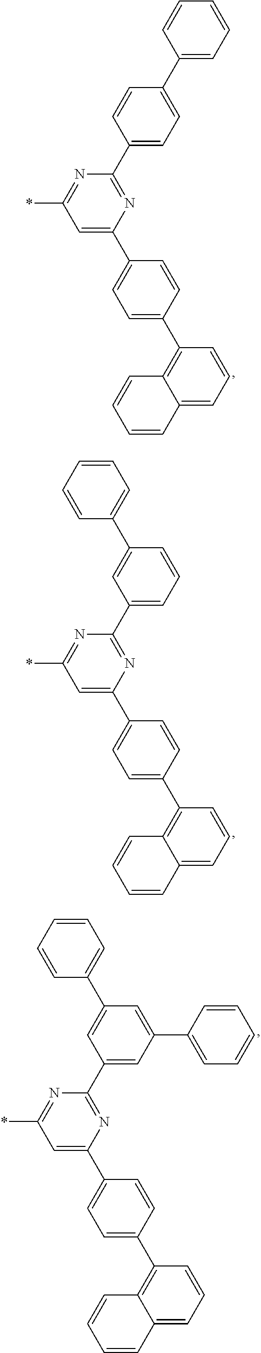

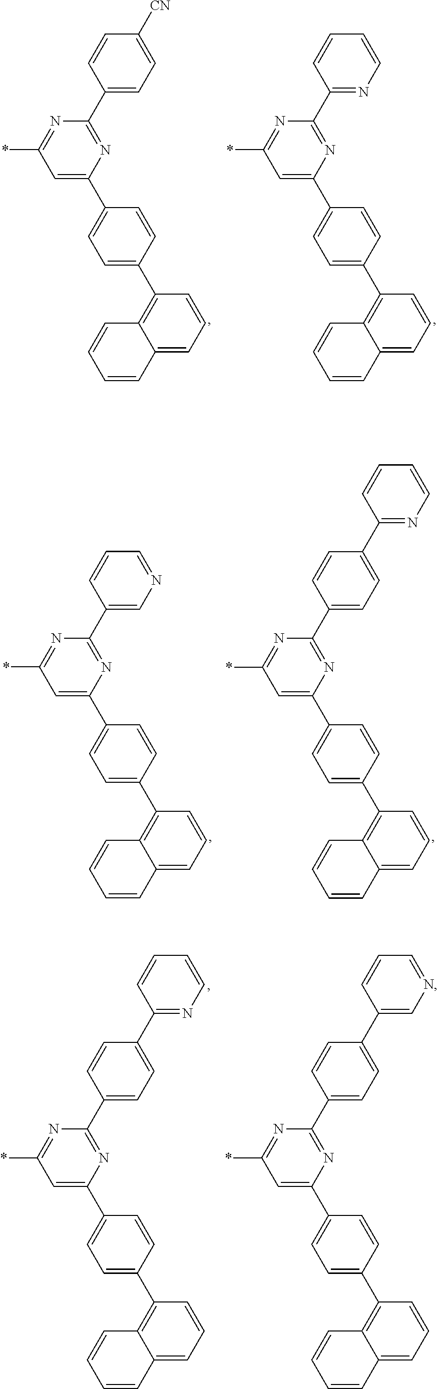 [SMILES: *c1cc(-c2ccc(-c3cccc4ccccc34)cc2)nc(-c2ccc(C#N)cc2)n1 ; *c1cc(-c2ccc(-c3cccc4ccccc34)cc2)nc(-c2ccccn2)n1 ; *c1cc(-c2ccc(-c3cccc4ccccc34)cc2)nc(-c2cccnc2)n1 ; *c1cc(-c2ccc(-c3cccc4ccccc34)cc2)nc(-c2ccc(-c3ccccn3)cc2)n1 ; *c1cc(-c2ccc(-c3cccc4ccccc34)cc2)nc(-c2ccc(-c3ccccn3)cc2)n1 ; *c1cc(-c2ccc(-c3cccc4ccccc34)cc2)nc(-c2ccc(-c3cccnc3)cc2)n1]

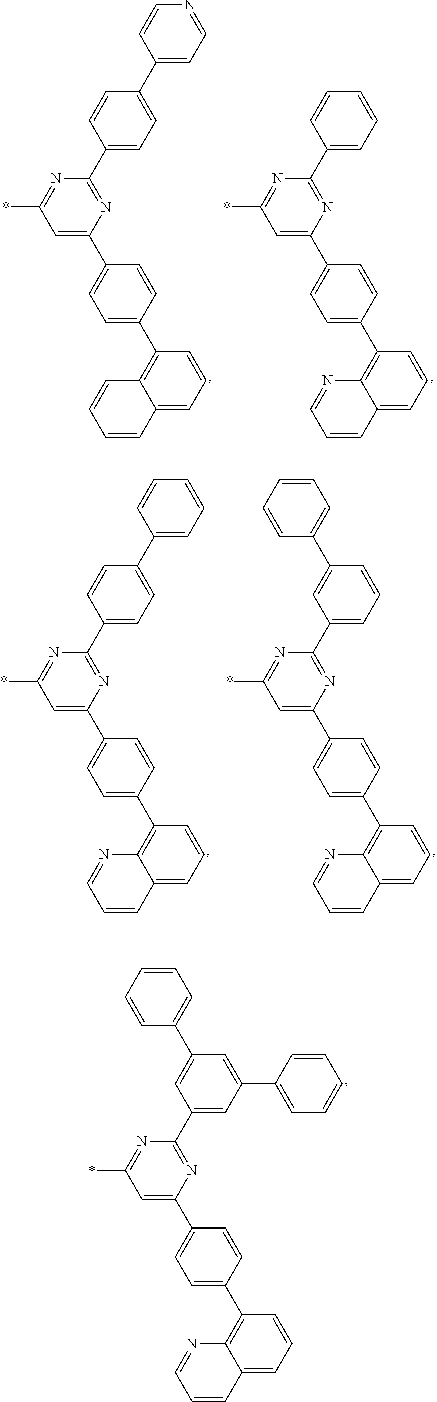

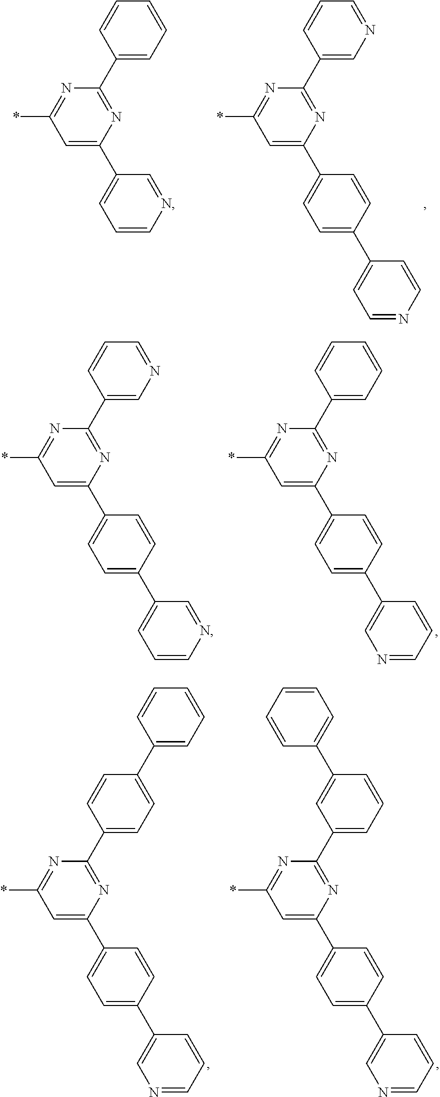

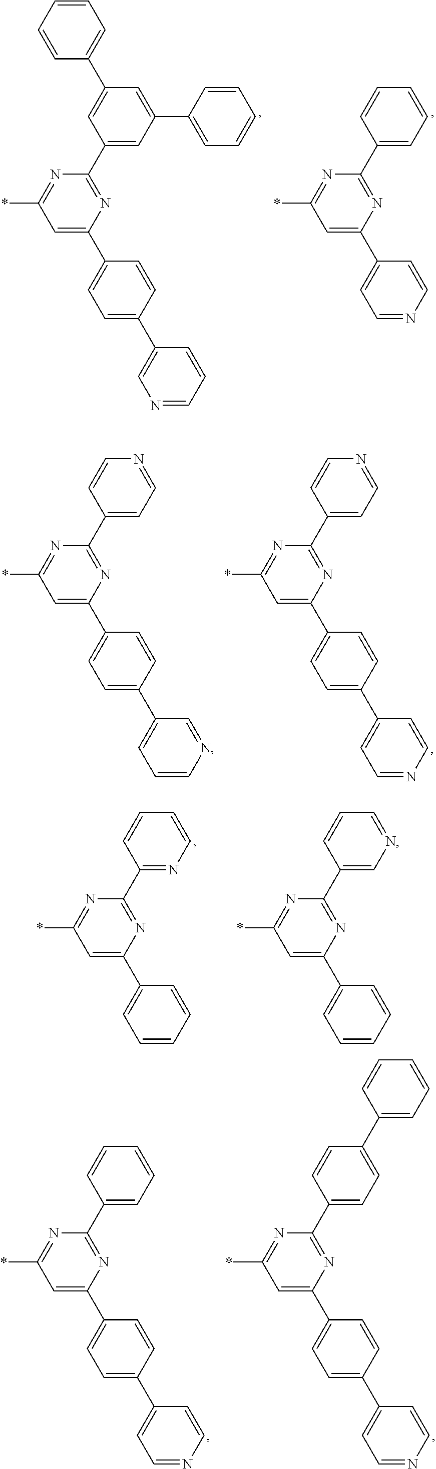

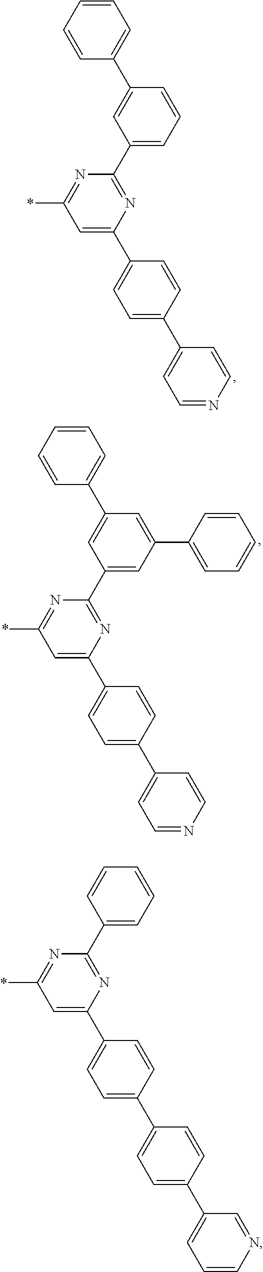

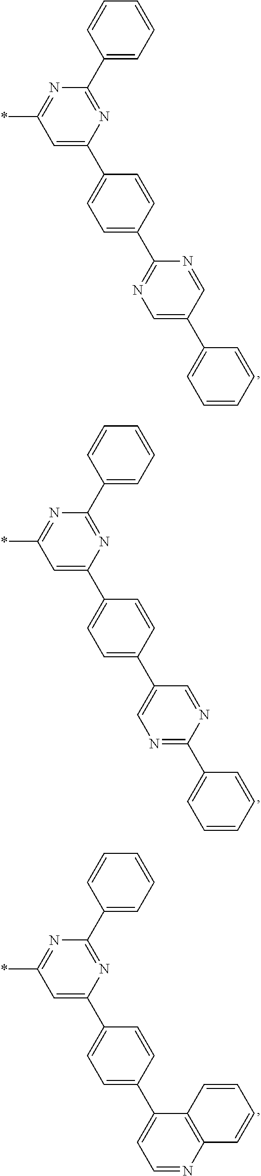 [SMILES: *c1cc(-c2ccc(-c3ncc(-c4ccccc4)cn3)cc2)nc(-c2ccccc2)n1 ; *c1cc(-c2ccc(-c3cnc(-c4ccccc4)nc3)cc2)nc(-c2ccccc2)n1 ; *c1cc(-c2ccc(-c3ccnc4ccccc34)cc2)nc(-c2ccccc2)n1]

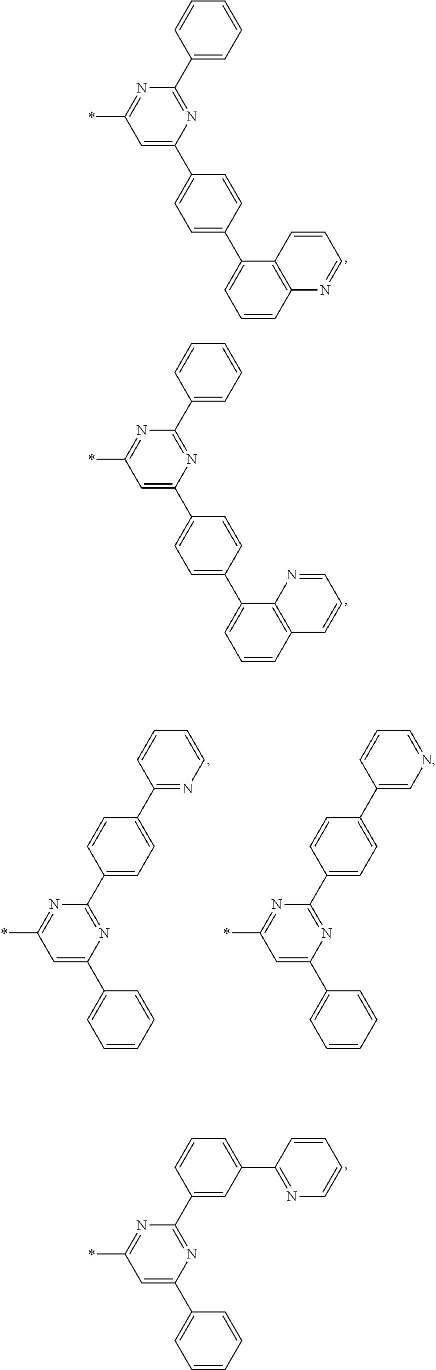

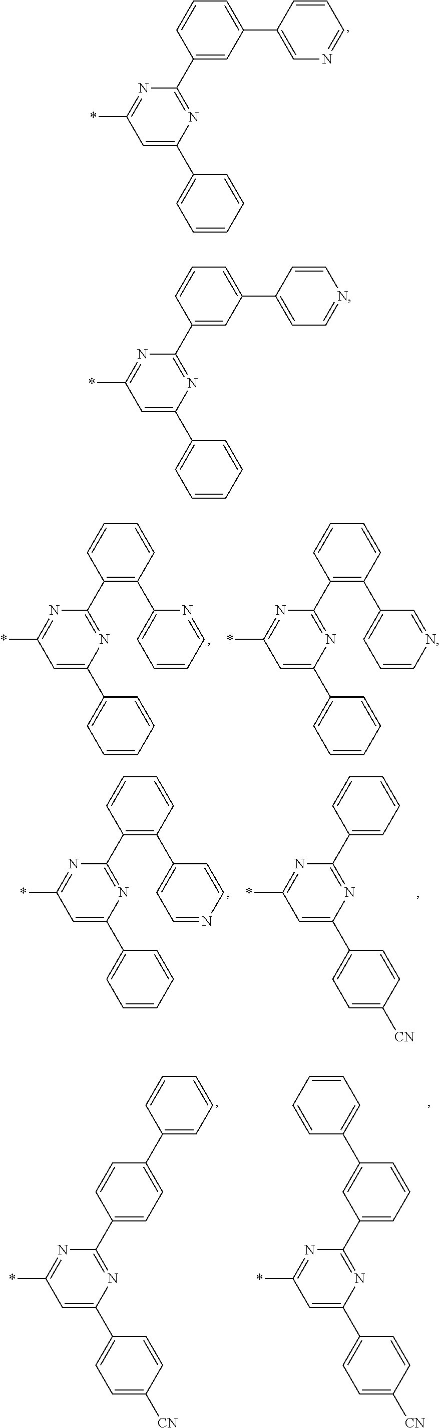

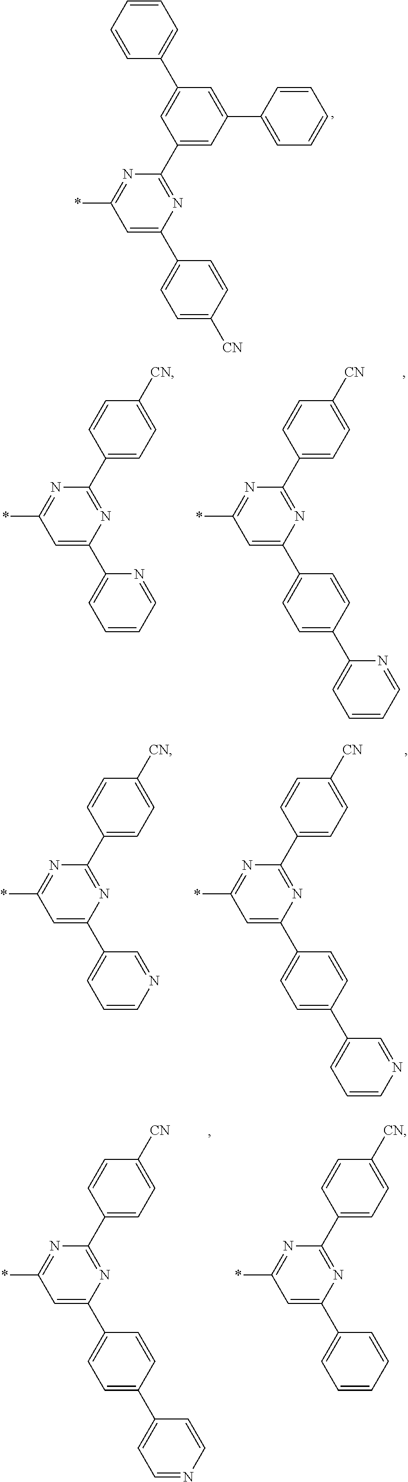

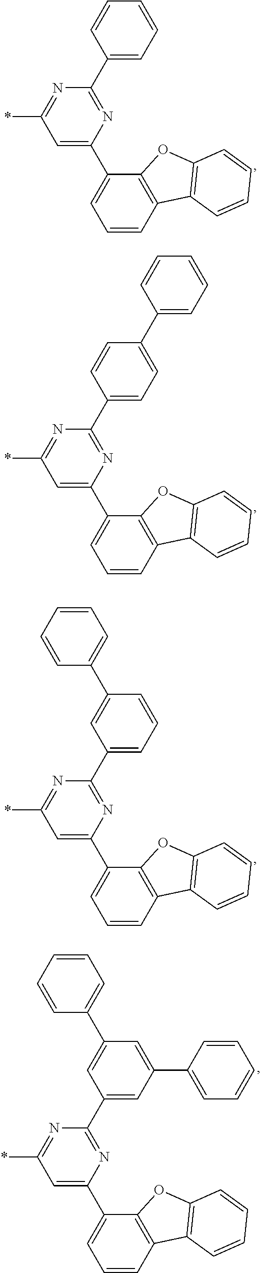

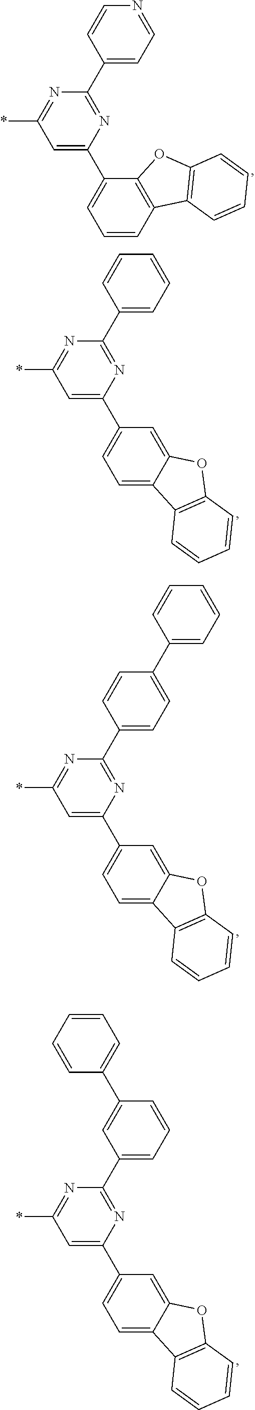

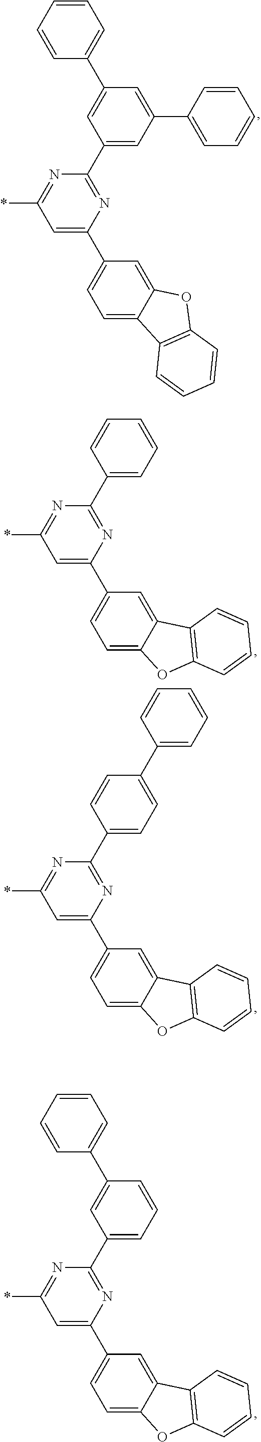

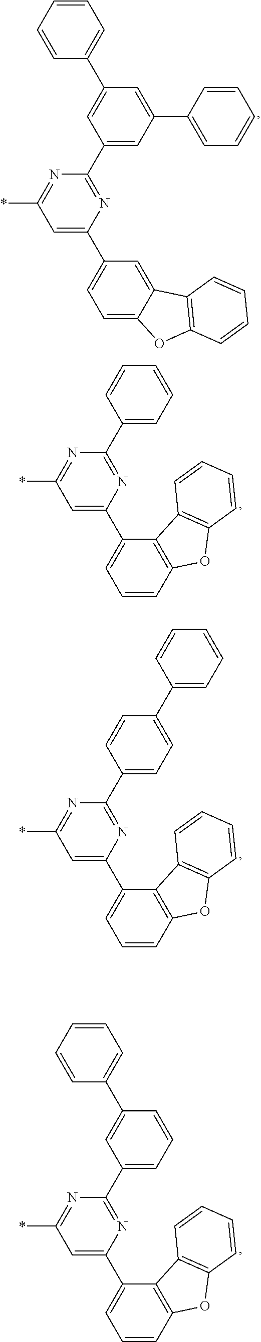

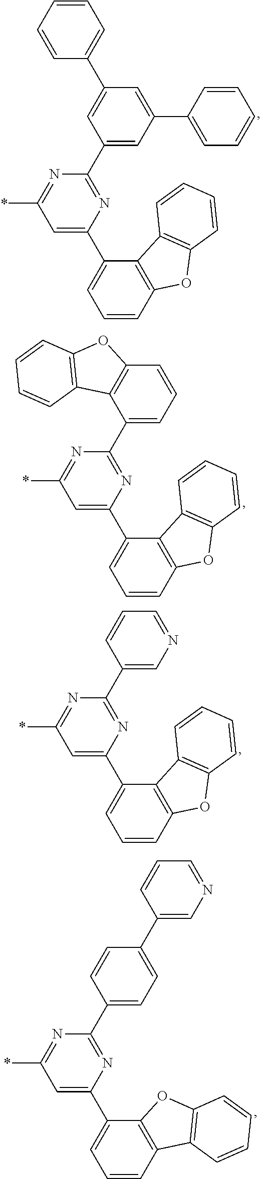

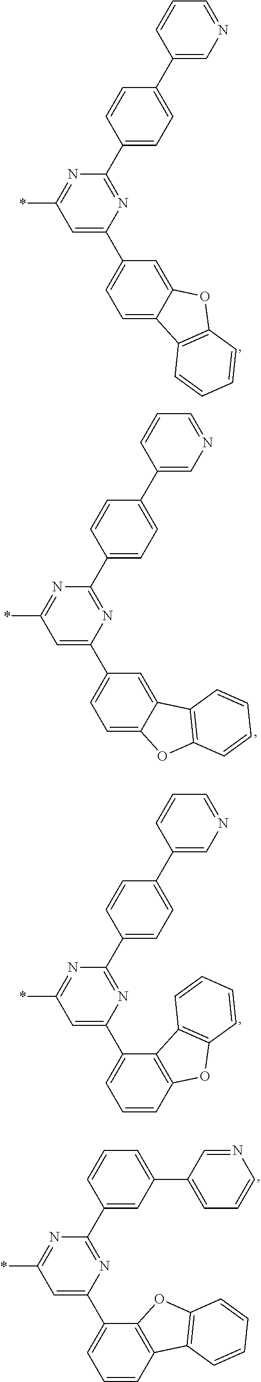

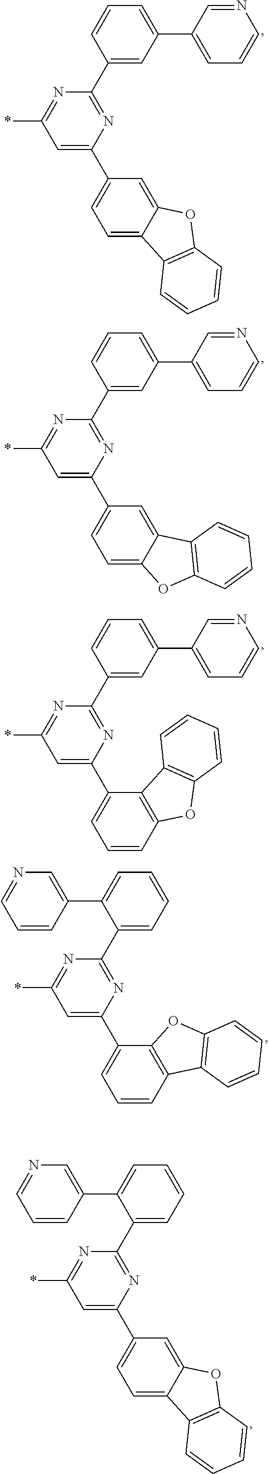

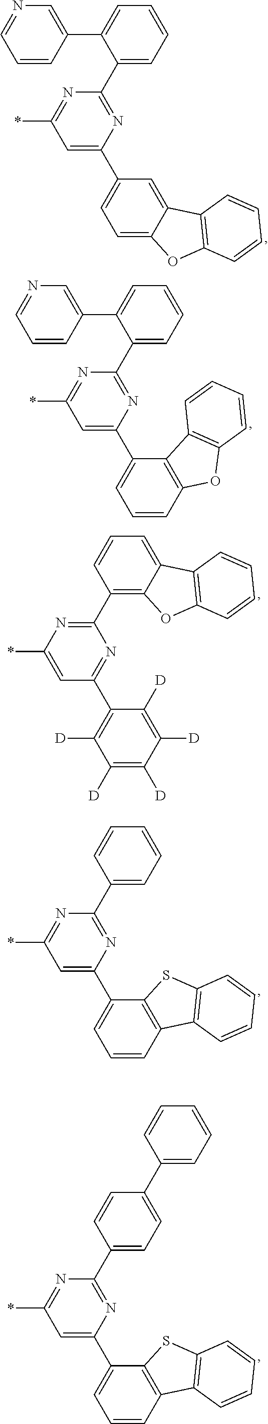

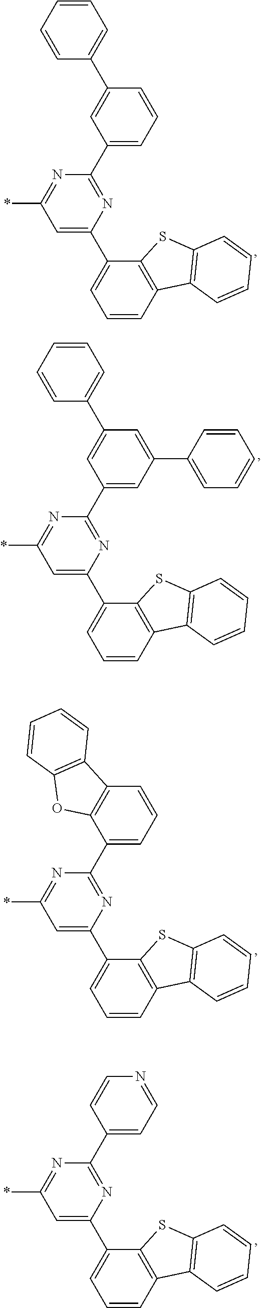

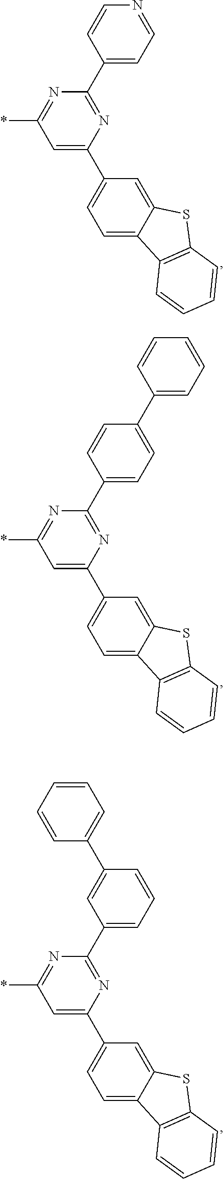

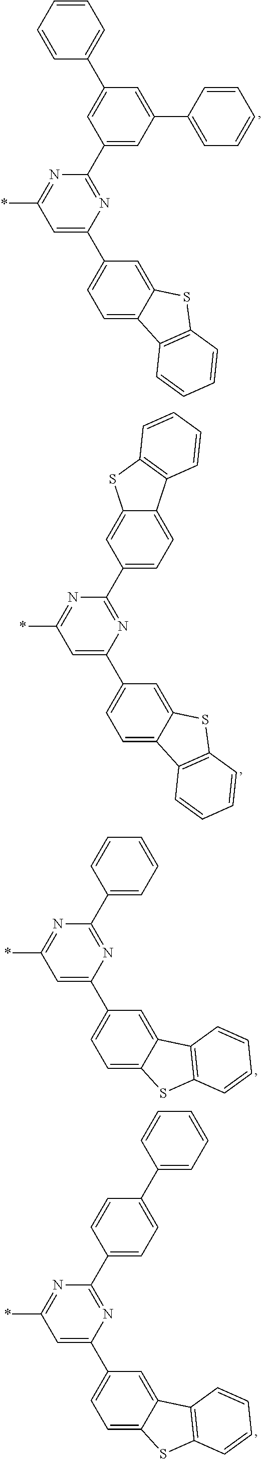

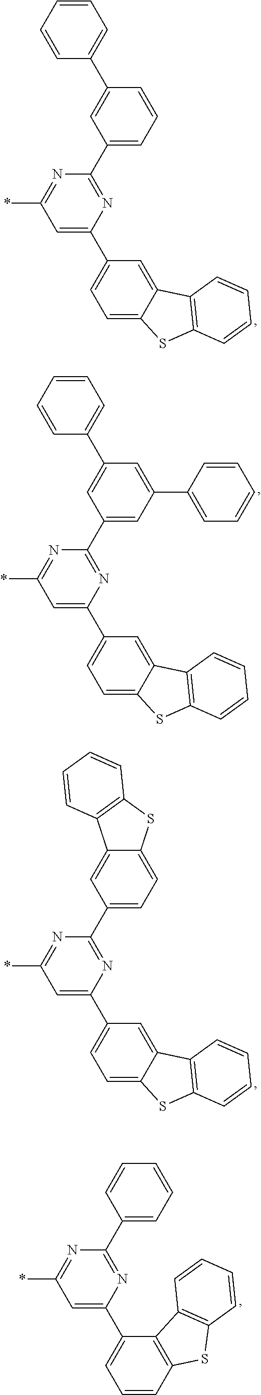 [SMILES: *c1cc(-c2ccc3sc4ccccc4c3c2)nc(-c2cccc(-c3ccccc3)c2)n1 ; *c1cc(-c2ccc3sc4ccccc4c3c2)nc(-c2cc(-c3ccccc3)cc(-c3ccccc3)c2)n1 ; *c1cc(-c2ccc3sc4ccccc4c3c2)nc(-c2ccc3sc4ccccc4c3c2)n1 ; *c1cc(-c2cccc3sc4ccccc4c23)nc(-c2ccccc2)n1]

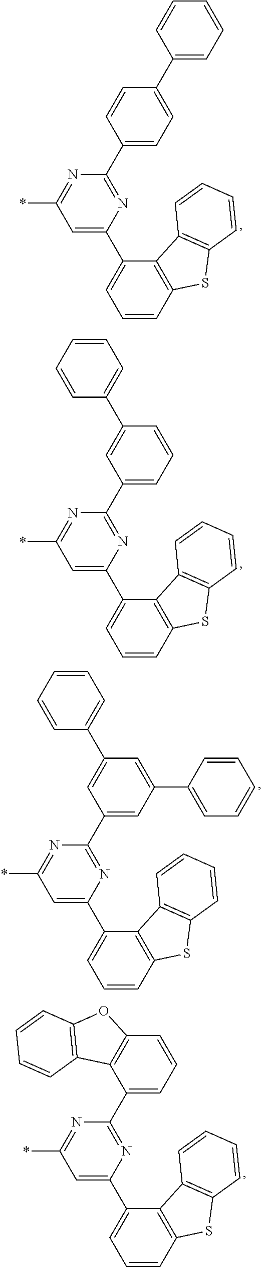

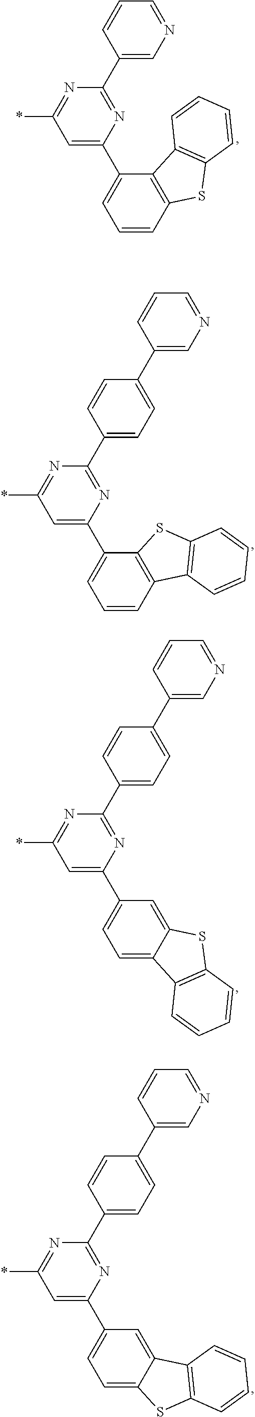

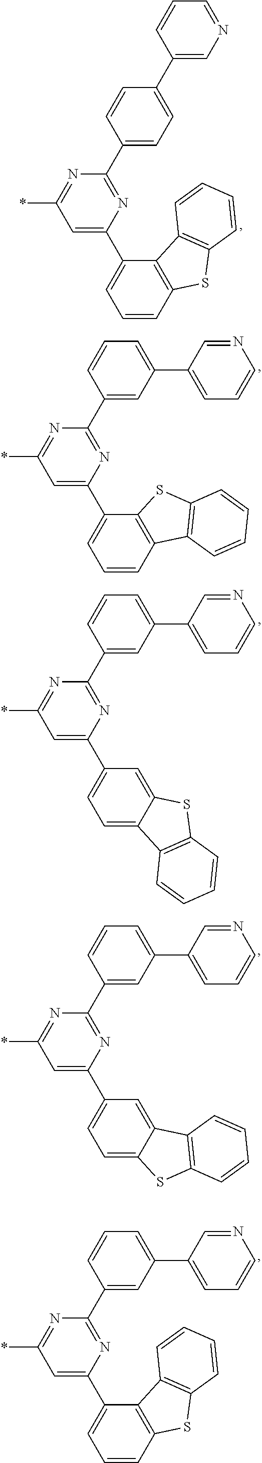

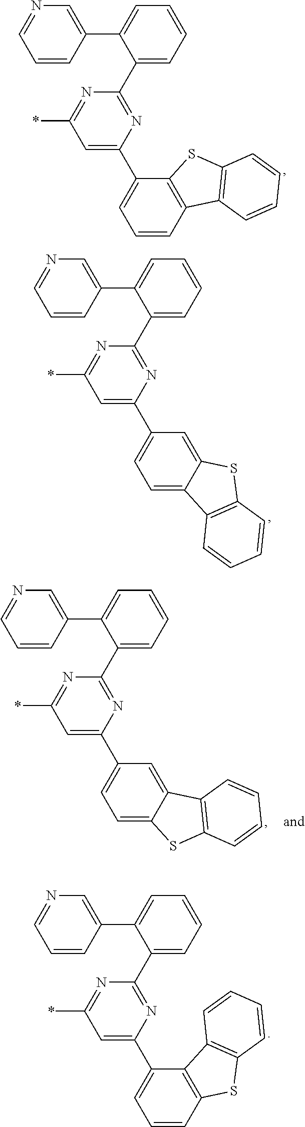

11. The compound as claimed in claim 1, wherein the compound is selected from the group consisting of:

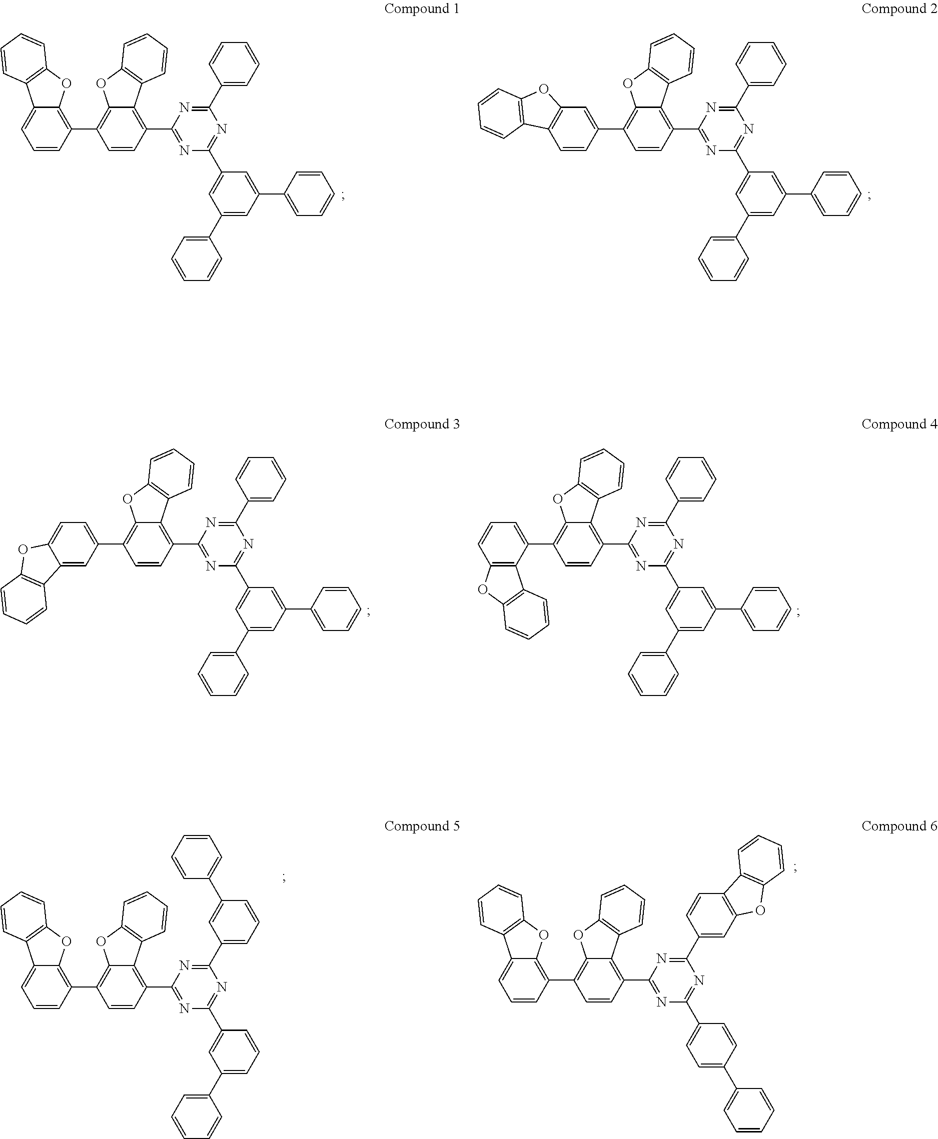

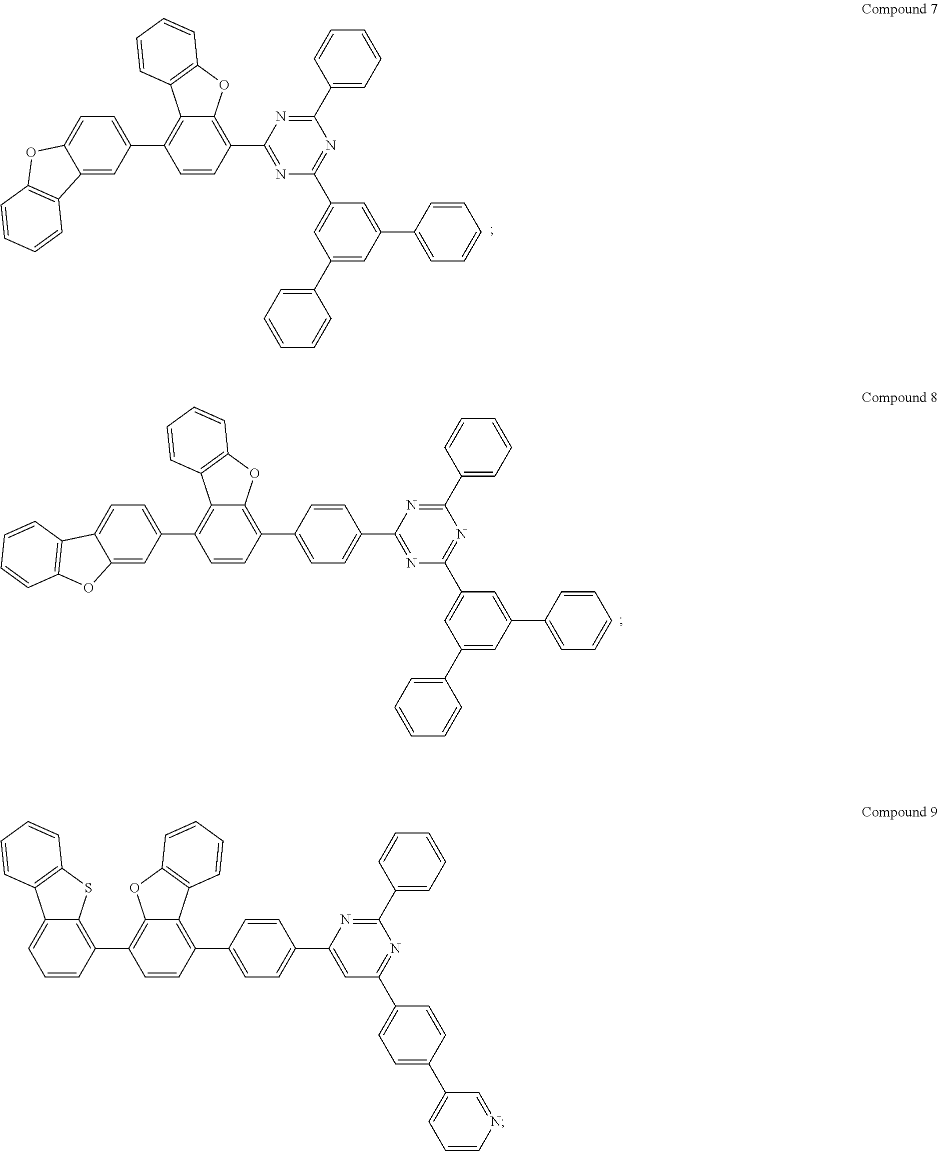 [SMILES: c1ccc(-c2cc(-c3ccccc3)cc(-c3nc(-c4ccccc4)nc(-c4ccc(-c5ccc6oc7ccccc7c6c5)c5c4oc4ccccc45)n3)c2)cc1 ; c1ccc(-c2cc(-c3ccccc3)cc(-c3nc(-c4ccccc4)nc(-c4ccc(-c5ccc(-c6ccc7c(c6)oc6ccccc67)c6c5oc5ccccc56)cc4)n3)c2)cc1 ; c1ccc(-c2nc(-c3ccc(-c4cccnc4)cc3)cc(-c3ccc(-c4ccc(-c5cccc6c5sc5ccccc56)c5oc6ccccc6c45)cc3)n2)cc1]

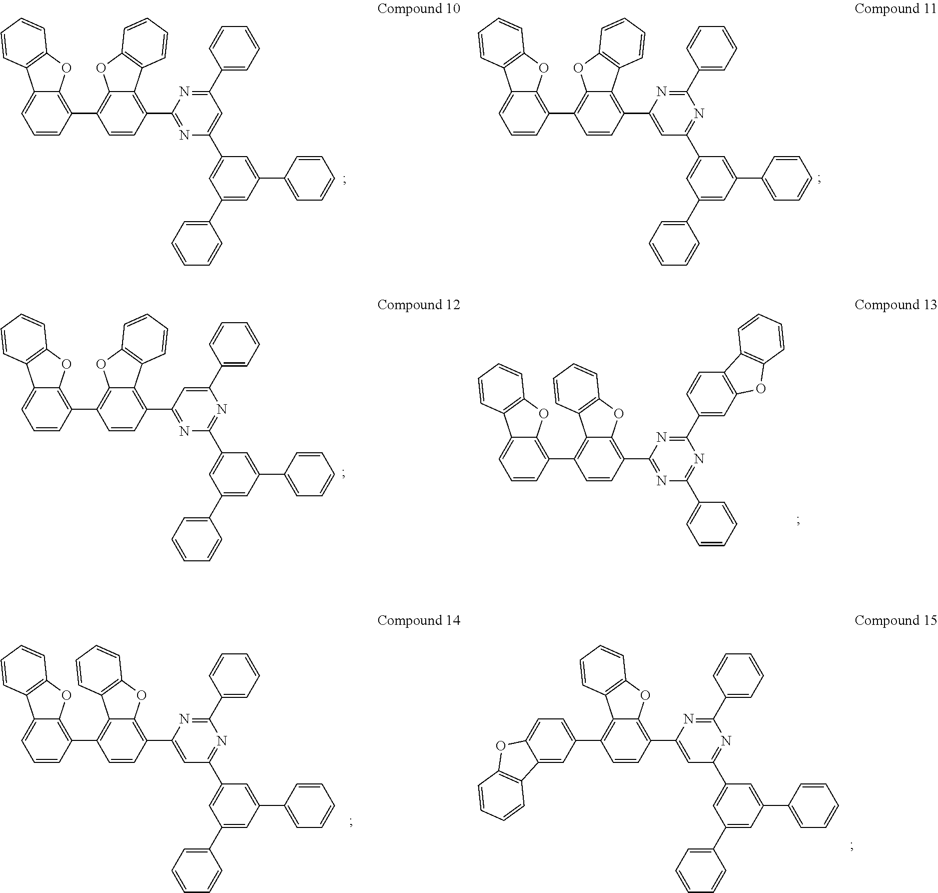

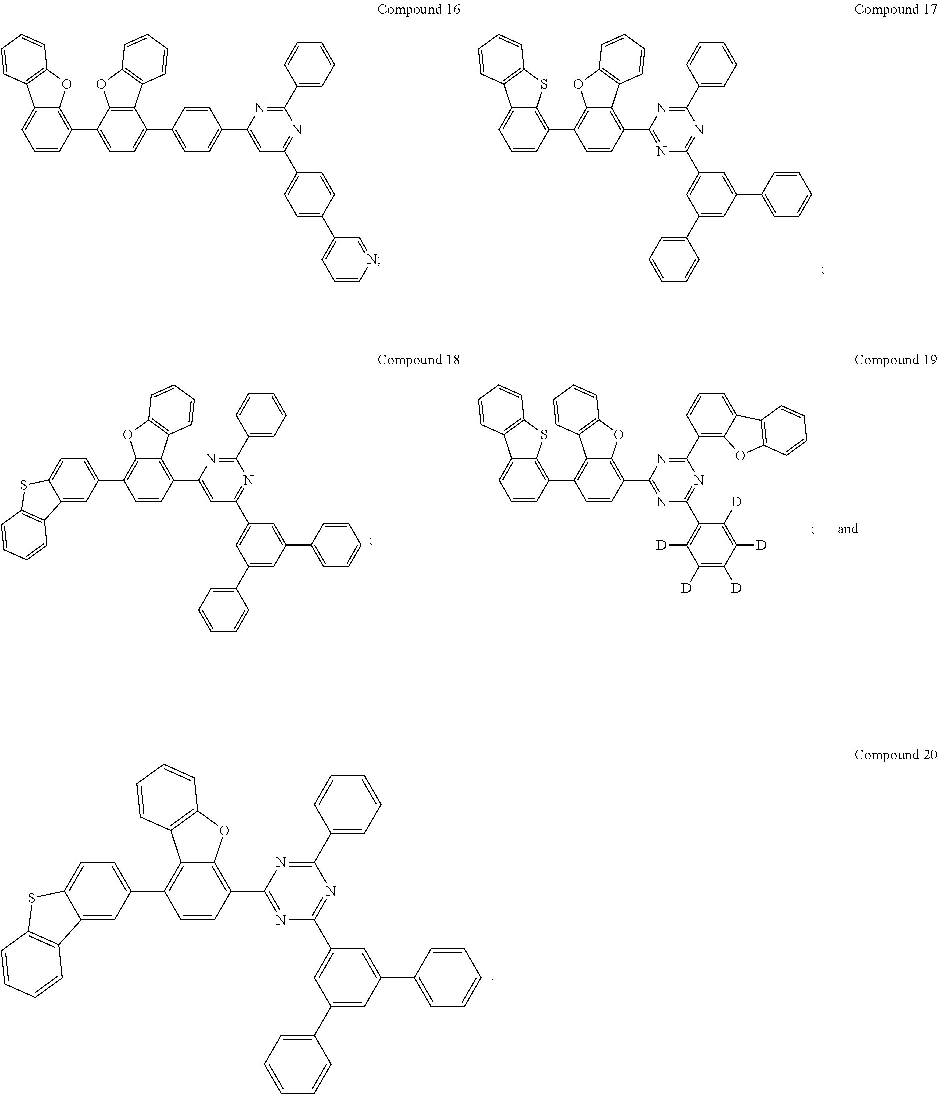

12. An organic electronic device, comprising a first electrode, a second electrode, and an organic layer disposed between the first electrode and the second electrode, wherein the organic layer comprises the compound as claimed in claim 1.

13. The organic electronic device as claimed in claim 12, wherein the organic electronic device is an organic light emitting device.

14. The organic electronic device as claimed in claim 13, wherein the organic light emitting device comprises:
a hole injection layer formed on the first electrode;
a hole transport layer formed on the hole injection layer;
an emission layer formed on the hole transport layer;
a first electron transport layer formed above the emission layer,
wherein the organic layer is the first electron transport layer; and
an electron injection layer formed between the first electron transport layer and the second electrode.

15. The organic electronic device as claimed in claim 14, wherein the organic light emitting device comprises a second electron transport layer formed between the emission layer and the first electron transport layer.

16. The organic electronic device as claimed in claim 14, wherein the organic light emitting device comprises a second electron transport layer formed between the electron injection layer and the first electron transport layer.
